(19) United States
(12) Patent Application Publication
(10) Pub. No.: US 2004/0072156 A1
(43) Pub. Date: Apr. 15, 2004

(54) Detection of genetic polymorphisms

(75) Inventors: Yusuke Nakamura, Yokohama-shi (JP); Akihiro Sekine, Tokyo (JP); Aritoshi Ilda, Kawasaki-shi (JP); Susumu Saito, Tokyo (JP)

Correspondence Address: MEDLEN & CARROLL, LLP, 101 Howard Street, Suite 350, San Francisco, CA, 94105, US

(73) Assignee: Riken Institute of Physical and Chemical Research

(21) Appl. No.: 10/035,833

(22) Filed: Dec. 27, 2001

(30) Foreign Application Priority Data
Dec. 27, 2000 (JP) 2000-399,443
May 2, 2001 (JP) 2001-135,256
Aug. 27, 2001 (JP) 2001-256,862
Dec. 26, 2001 (JP) 2001-395196
Dec. 27, 2001 (WO) PCT/JP01/11592

(51) Int. Cl. C12Q 1/68

(52) U.S. Cl. 435/6

(57) ABSTRACT

The present invention relates to genetic polymorphism data, compositions and methods for detecting genetic polymorphisms, methods for evaluating drugs using genetic polymorphisms and screening methods for drugs.

The present invention claims priority to Japanese Patent Application Ser. Nos. 2000-399,443 filed Dec. 27, 2000, 2001-135,256 filed May 2, 2001, 2001-256,862 filed Aug. 27, 2001, and 2001-(awaiting serial number) filed December 2001, each of which was filed with the Commissioner of the Japanese Patent Office. Right of priority under 35 U.S.C. 119 is claimed from these Japanese patent applications under the Paris Convention for the Protection of Industrial Property. The present invention also claims priority to PCT application (awaiting serial number) filed Dec. 27, 2001 in the Japanese receiving office. Each of these applications are herein incorporated by reference in their entireties.

### FIELD OF THE INVENTION

The present invention relates to genetic polymorphism data, compositions and methods for detecting genetic polymorphisms, methods for evaluating drugs using genetic polymorphisms and screening methods for drugs.

### BACKGROUND

Human beings come in all shapes and sizes, and over three billion genetic codes are located in somewhat different sites in each human being. Individual DNA sequence variations in the human genome are known to directly cause specific diseases or conditions, to predispose certain individuals to specific diseases or conditions, and to affect responses of individuals to treatments such as drugs. Such variations also modulate the severity or progression of many diseases. Additionally, DNA sequences vary between populations. Therefore, determining DNA sequence variations in the human genome is useful for making accurate diagnoses, for finding suitable therapies, and for understanding the relationship between genome variations and environmental factors in the pathogenesis of diseases, the prevalence of conditions and the efficacy of therapies. function. Analysis of genetic polymorphisms such as SNPs allows for the selection of drugs and the development of treatment protocols tailored to each individual patient (so-called “personalized” medical treatments). Instead of the using trial-and-error methods of matching patients with the right drugs, doctors may, for example, be able to analyze a patient's genetic profile and prescribe the best available drug therapy from the beginning. Not only would this take the guesswork out of finding the right drug, it would reduce the likelihood of adverse reactions, thus increasing safety.

### SUMMARY OF THE INVENTION

The present invention identifies genetic polymorphisms relating to genes associated with drug metabolism. In some embodiments, the present invention provides methods for determining variations in sequences and genes associated with drug-metabolizing enzymes. In preferred embodiments, the present invention provides methods for collecting genetic polymorphism data for use in evaluating the effectiveness and safety of a drug based on the data, and screening drugs using the data. In some preferred embodiments, the polymorphisms of the present invention are used to evaluate a causal relationship between the genetic make-up of a patient and a response to an administered drug.

The present invention relates to genes encoding enzymes associated with drug metabolism (drug metabolizing enzymes, or DMEs). In particular, the present invention relates to sequence variations associated with variations in DMEs. In some embodiments, variations occur in coding regions of DMEs, such as may alter a function of the DMEs, (e.g., by increasing or decreasing its level of activity, or shifting its activity to an alternative target or function). In other embodiments, the variations occur in non-coding regions of the genome, such as may alter expression of a DME (e.g., increasing or decreasing the amount of an enzyme produced in a cell) or processing of an RNA transcript encoding a DME (e.g., by altering splicing).

In some embodiments, the present invention provides methods for detecting DME-related sequence variations. In some preferred embodiments, the methods of the present invention are used to create a profile of DME-related polymorphisms in a test subject.

In other embodiments, the present invention provides isolated nucleic acid sequences encoding variant DMEs. For example, the present invention provides a recombinant DNA vector comprising DNA having a nucleotide sequence encoding a variant DME, the nucleotide sequence comprising a sequence including, but not limited to, SEQ ID NOS:1-3360 and 3361-7669, and substantially similar sequences. In a preferred embodiment, the invention provides a host cell transformed with a recombinant DNA vector comprising DNA having a nucleotide sequence encoding a variant DME. The invention is not limited by the nature of the host cell employed. The art is well aware of expression vectors suitable for the expression of nucleotide sequences encoding variant DMEs that can be expressed in a variety of prokaryotic and eukaryotic host cells. In some preferred embodiments, the host cell is a eukaryotic cell grown in culture, such as for use in in vitro drug screening (e.g., by monitoring the expression of genes associated with the pathways targeted by a particular test drug). In other preferred embodiments, the host cell is in vivo.

The present invention provides systems and methods for detection of polymorphisms associated with genes encoding enzymes associated with drug metabolism. The present invention is not limited in the nature of the detection assay used for detection or identification of such polymorphisms. Such detection assays include, but are not limited to, hybridization methods and array technologies (e.g., technologies available from Aclara BioSciences, Haywood, Calif.; Affymetrix, Santa Clara, Calif.; Agilent Technologies, Inc., Palo Alto, Calif.; Aviva Biosciences Corp., San Diego, Calif.; Caliper Technologies Corp., Palo Alto, Calif.; Celera, Rockville, Md.; CuraGen Corp., New Haven, Conn.; Hyseq Inc., Sunnyvale, Calif.; Illumina, Inc., San Diego, Calif.; Incyte Genomics, Palo Alto, Calif.; Motorola BioChip Systems; Nanogen, San Diego, Calif.; Orchid BioSciences, Inc., Princeton, N.J.; Applera Corp., Foster City, Calif.; Rosetta Inpharmatics, Kirkland, Wash.; and Sequenom, San Diego, Calif.); polymerase chain reaction-based methods (e.g.,TAQMAN, Applera Corp., GENECODE system, EraGen, Middleton, Wis.); branched hybridization methods; enzyme mismatch cleavage methods; NASBA; sandwich hybridization methods; methods employing molecular beacons; ligase chain reactions, and the like.

Methods of the present invention find application in improving the drug discovery and approval processes. For example, the costs and risks of drug development may be reduced if only those persons capable of responding to a drug are selected for clinical trials. In addition, previously failed drug candidates may be revived as they are matched with more appropriate patient populations. Decreases in the number of adverse drug reactions, the number of failed drug trials, the time it takes to get a drug approved, the length of time patients are on medication, the number of medications patients must take to find an effective therapy, and an increase in the range of possible drug targets will promote a net decrease in the cost of health care.

Thus, in some embodiments, the present invention provides a method of identifying individuals having a polymorphism, comprising providing nucleic acid from a subject; and detecting the presence of at least one polymorphism in said nucleic acid, said at least one polymorphism including, but not limited to, polymorphisms found in SEQ ID Nos:1-3360 and 3361-7669. In some embodiments, the method further provides the step of providing a prognosis (e.g., a genotype relative risk or a population attributable risk) to the subject based on the presence or absence of the at least one polymorphism. In some embodiments, the detecting step is carried out using a detection assay including, but not limited to, a hybridization assay, a TAQMAN assay, an invasive cleavage assay, use of mass spectroscopy, a microarray, a polymerase chain reaction, a rolling circle extension assay, a sequencing assay, a hybridization assay employing a probe complementary to a polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay.

The present invention also provides a nucleic acid (e.g., a gene, a probe, a primer, etc.) comprising a sequence selected from the group consisting of SEQ ID NO:1-3360 and 3361-7669 or complements thereof. In some embodiments, the nucleic acid molecule comprises a label. In some embodiments, the nucleic acid is attached to a solid support (e.g., as part of a microarray). The present invention also provides vectors comprising the nucleic acid and host cell comprising the vector, as well as polypeptide encoded by the nucleic acid. Methods of producing and purifying polypeptides are well known in the art.

The present invention further provides kits for detecting a polymorphism, comprising at least one reagent that specifically detects a polymorphism in a sequence including, but not limited to, SEQ ID Nos:1-3360 and 3361-7669. In some embodiments, the kit further comprising instructions for determining whether the subject is at increased risk of having a drug metabolism disorder. In some embodiments, the at least one reagent comprises a nucleic acid probe. The kits can be configured for a variety of uses including, but not limited to, use as an in vitro diagnostic detection assay, an analyte specific reagent detection assay, and a research-use-only detection assay.

The present invention also provides a method for screening subjects for genetic nmarkers associated with drug metabolizing enzyme(s), comprising: a) providing a biological sample comprising a nucleic acid from a subject; b) testing the nucleic acid for a polymorphism in a genetic marker associated with a drug metabolizing enzyme, said genetic marker comprising one or more nucleotide polymorphisms designated by n, said n selected from a base substitution, an insertion, or a deletion found in a sequence selected from the group consisting of SEQ ID Nos:1-3360 and 3361-7669. The present invention is not limited by the source of the nucleic acid. In some embodiments, the biological sample comprises blood, saliva, amniotic fluid, and tissue. In some embodiments, the subject is a human. In some preferred embodiments, the nucleic acid comprises DNA and/or RNA.

The present invention further provides a composition comprising an array of detection assays, said array comprising a plurality of drug metabolizing enzyme nucleotide polymorphism detection assays, one or more of said detection assays being capable of detecting one or more nucleotide polymorphisms designated by n in SEQ ID Nos:1-3360 and 3361-7669, wherein n represents a base substitution, insertion, or deletion compared to a wild-type sequence.

The present invention also provides a composition comprising a detection probe for determining the presense or absence a single nucleotide polymorphism in a gene encoding a drug metabolizing enzyme, said gene comprising a sequence selected from the group consisting of SEQ ID Nos:1-3360 and 3361-7669.

The present invention further provides a method of determining the effectiveness of or side-effect of a drug or treatment protocol, comprising; a) administering a drug or treatment protocol to one or more subjects; b) obtaining nucleic acid from said one or more subjects; c) using a detection assay to detect the presence of at least one polymorphism in said nucleic acid from said one or more of subjects, said at least one polymorphism selected from the group consisting of polymorphisms found in SEQ ID Nos:1-3360 and 3361-7669; and d) assigning an effectiveness rating, side-effect rating, or score for said drug or treatment protocol based upon a result of one or more said detection assays (See e.g., Toxicology Testing Handbook: Principles, Applications, and Data Interpretation, ed. Jacobson-Kram and Keller, 2001, herein incorporated by reference in its entirety).

The present invention also provides a method of prescribing a drug to or treatment protocol for a subject, comprising; providing nucleic acid from said subject; using a detection assay to detect the presence of at least one polymorphism in the nucleic acid, said at least one polymorphism selected from the group consisting of polymorphisms found in SEQ ID Nos:1-3360 and 3361-7669; and, prescribing said drug or treatment protocol based upon the result of said detection assay.

The present invention further provides a method for generating assay data comprising: obtaining a sample from a subject containing nucleic acid; transferring said sample to a laboratory; and receiving data from said laboratory, wherein said data corresponds to the presence of at least one polymorphism in said nucleic acid, said at least one polymorphism selected from the group consisting of polymorphisms found in SEQ ID Nos:1-3360 and 3361-7669. The present further provides data sets generated by this method.

### DEFINITIONS

To facilitate an understanding of the present invention, a number of terms and phrases are defined below:

As used herein, the terms “complementary” or “complementarity” are used in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand. Nucleotide analogs used to form non-standard base pairs, whether with another nucleotide analog (e.g., an IsoC/IsoG base pair), or with a naturally occurring nucleotide (e.g., as described in U.S. Pat. No. 5,912,340, herein incorporated by reference in its entirety) are also considered to be complementary to a base pairing partner within the meaning this definition.

The term “homology” and “homologous” refers to a degree of identity. There may be partial homology or complete homology. A partially homologous sequence is one that is less than 100% identical to another sequence.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, i.e., a nucleic acid having a complementary nucleotide sequence. The ability of two polymers of nucleic acid containing complementary sequences to Find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modem biology.

With regard to complementarity, it is important for some diagnostic applications to determine whether the hybridization represents complete or partial complementarity. For example, where it is desired to detect simply the presence or absence of a foreign DNA sequence, it is only important that the hybridization method ensures hybridization when the relevant sequence is present; conditions can be selected where both partially complementary probes and completely complementary probes will hybridize. Other diagnostic applications, however, may require that the hybridization method distinguish between partial and complete complementarity. It may be of interest to detect genetic polymorphisms. For example, human hemoglobin is composed, in part, of four polypeptide chains. Two of these chains are identical chains of 141 amino acids (alpha chains) and two of these chains are identical chains of 146 amino acids (beta chains). The gene encoding the beta chain is known to exhibit polymorphism. The normal allele encodes a beta chain having glutamic acid at the sixth position. The mutant allele encodes a beta chain having valine at the sixth position. This difference in amino acids has a profound (most profound when the individual is homozygous for the mutant allele) physiological impact known clinically as sickle cell anemia. It is well known that the genetic basis of the amino acid change involves a single base difference between the normal allele DNA sequence and the mutant allele DNA sequence.

The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

As used herein, the term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr. Thermodynamics and NMR of internal G.T mismatches in DNA. Biochemistry 36, 10581-94 (1997) include more sophisticated computations which take structural and environmental, as well as sequence characteristics into account for the calculation of Tₘ.

As used herein the term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required when it is desired that nucleic acids that are not completely complementary to one another be hybridized or annealed together.

“High stringency conditions” when used in reference to nucleic acid hybridization comprise conditions equivalent to binding or hybridization at 42 C in a solution consisting of 5× SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1× SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5× SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0× SSPE, 1.0% SDS at 42 C when a probe of about 500 nucleotides in length is employed.

“Low stringency conditions” comprise conditions equivalent to binding or hybridization at 42 C in a solution consisting of 5× SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5× SSPE, 0.1% SDS at 42 C when a probe of about 500 nucleotides in length is employed.

The term “gene” refers to a DNA sequence that comprises control and coding sequences necessary for the production of an RNA having a non-coding function (e.g., a ribosomal or transfer RNA), a polypeptide or a precursor. The RNA or polypeptide can be encoded by a full-length coding sequence or by any portion of the coding sequence so long as the desired activity or function is retained.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “oligonucleotide” as used herein is defined as a molecule comprising two or more deoxyribonucleotides or ribonucleotides, preferably at least 5 nucleotides, more preferably at least about 10-15 nucleotides and more preferably at least about 15 to 30 nucleotides. The exact size will depend on many factors, which in turn depend on the ultimate function or use of the oligonucleotide. The oligonucleotide may be generated in any manner, including chemical synthesis, DNA replication, reverse transcription, PCR, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The term “primer” refers to an oligonucleotide that is capable of acting as a point of initiation of synthesis when placed under conditions in which primer extension is initiated. An oligonucleotide “primer” may occur naturally, as in a purified restriction digest or may be produced synthetically.

A primer is selected to be “substantially” complementary to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template primer complex for synthesis of the extension product of the primer.

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable.

The term “signal” as used herein refers to any detectable effect, such as would be caused or provided by a label or an assay reaction.

As used herein, the term “detector” refers to a system or component of a system, e.g., an instrument (e.g. a camera, fluorimeter, charge-coupled device, scintillation counter, etc.) or a reactive medium (X-ray or camera film, pH indicator, etc.), that can convey to a user or to another component of a system (e.g., a computer or controller) the presence of a signal or effect. A detector can be a photometric or spectrophotometric system, which can detect ultraviolet, visible or infrared light, including fluorescence or chemiluminescence; a radiation detection system; a spectroscopic system such as nuclear magnetic resonance spectroscopy, mass spectrometry or surface enhanced Raman spectrometry; a system such as gel or capillary electrophoresis or gel exclusion chromatography; or other detection systems known in the art, or combinations thereof.

The term “sequence variation” as used herein refers to differences in nucleic acid sequence between two nucleic acids. For example, a wild-type structural gene and a mutant form of this wild-type structural gene may vary in sequence by the presence of single base substitutions and/or deletions or insertions of one or more nucleotides. These two forms of the structural gene are said to vary in sequence from one another. A second mutant form of the structural gene may exist. This second mutant form is said to vary in sequence from both the wild-type gene and the first mutant form of the gene.

The term “nucleotide analog” as used herein refers to modified or non-naturally occurring nucleotides such as 7-deaza purines (i.e., 7-deaza-dATP and 7-deaza-dGTP). Nucleotide analogs include base analogs and comprise modified forms of deoxyribonucleotides as well as ribonucleotides.

The term “polymorphism” refers to the coexistence of more than one form of a gene or portion thereof. A portion of a gene of which there are at least two different forms, i.e., two different nucleotide sequences,, is referred to as a “polymorphic region of a gene”. A polymorphic region can be a single nucleotide, the identity of which differs in different alleles. A polymorphic region can also be several nucleotides long.

A “polymorphic gene” refers to a gene having at least one polymorphic region.

The term “polymorphic locus” is a locus present in a population that shows variation between members of the population (e.g., the most common allele has a frequency of less than 0.95). In contrast, a “monomorphic locus” is a genetic locus at little or no variations seen between members of the population (generally taken to be a locus at which the most common allele exceeds a frequency of 0.95 in the gene pool of the population).

A “non-human animal” of the invention can include mammals such as rodents, non-human primates, sheep, goats, horses, dogs, cows, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying drugs that can affect processes, e.g., embryogenesis and tissue formation.

The term “operably linked” is intended to mean that the promoter is associated with the nucleic acid in such a manner as to facilitate transcription of the nucleic acid from the promoter.

The terms “protein”, “polypeptide” and “peptide” are used interchangeably herein when referring to a gene product.

The term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

A “regulatory element”, also termed herein “regulatory sequence” is intended to include elements which are capable of modulating transcription from a basic promoter and include elements such as enhancers and silencers. The term “enhancer”, also referred to herein as “enhancer element”, is intended to include regulatory elements capable of increasing, stimulating, or enhancing transcription from a basic promoter. The term “silencer”, also referred to herein as “silencer element” is intended to include regulatory elements capable of decreasing, inhibiting, or repressing transcription from a basic promoter. Regulatory elements are typically present in 5′ flanking regions of genes. However, regulatory elements have also been shown to be present in other regions of a gene, in particular in introns. Regulatory elements may also be present downstream of coding regions. Thus, it is possible that DMI genes have regulatory elements located in introns, exons, coding regions, and 3′ flanking sequences. Such regulatory elements are also intended to be encompassed by the present invention and polymorphisms in such elements can be identified by any of the assays that can be used to identify polymorphisms in regulatory elements in 5′ flanking regions of genes.

The term “regulatory element” further encompasses “tissue specific” regulatory elements, i.e., regulatory elements that affect expression of a DME gene preferentially in specific cells (e.g., cells of a specific tissue). Gene expression occurs preferentially in a specific cell if expression in this cell type is significantly higher than expression in other cell types. The term “regulatory element” also encompasses non-tissue specific regulatory elements, i.e., regulatory elements that are active in most cell types. Furthermore, a regulatory element can be a constitutive regulatory element, i.e., a regulatory element that constitutively regulates transcription, as opposed to a regulatory element that is inducible, i.e., a regulatory element which is active primarily in response to a stimulus. A stimulus can be, e.g., a molecule, such as a hormone, cytokine, heavy metal, phorbol ester, cyclic AMP (cAMP), or retinoic acid.

As used herein, the term “transfection” means the introduction of a nucleic acid, e.g., an expression vector, into a recipient cell by nucleic acid-mediated gene transfer. The term “transduction” is generally used herein when the transfection with a nucleic acid is by viral delivery of the nucleic acid. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the recombinant protein is disrupted.

As used herein, the term “transgene” refers to a nucleic acid sequence that has been introduced into a cell. Daughter cells deriving from a cell in which a transgene has been introduced are also said to contain the transgene (unless it has been deleted). A transgene can encode, e.g., a polypeptide, or an antisense transcript, partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). Alternatively, a transgene can also be present in an episome. A transgene can include one or more transcriptional regulatory sequence and any other nucleic acid, (e.g. intron), that may be necessary for optimal expression of a selected nucleic acid.

A “transgenic animal” refers to any animal, preferably a non-human animal, e.g. a mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of a protein, e.g. either agonistic or antagonistic forms. However, transgenic animals in which the recombinant gene is silent are also contemplated. Moreover, “transgenic animal” also includes those recombinant animals in which gene disruption of one or more genes is caused by human intervention, including both recombination and antisense techniques.

The term “treating” as used herein is intended to encompass curing as well as ameliorating at least one symptom of the condition or disease.

The term “sample” in the present specification and claims is used in its broadest sense. On the one hand it is meant to include a biological (e.g., human) specimen. On the other hand, a sample may include a specimen of synthetic origin.

Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc.

The term “source of target nucleic acid” refers to any sample that contains or is suspected to contain nucleic acids (RNA or DNA). Particularly preferred sources of target nucleic acids are biological samples including, but not limited to blood, saliva, cerebral spinal fluid, pleural fluid, milk, lymph, sputum and semen.

The term “polymerization means” or “polymerization agent” refers to any agent capable of facilitating the addition of nucleoside triphosphates to an oligonucleotide. Preferred polymerization means comprise DNA and RNA polymerases.

The term “ligation means” or “ligation agent” refers to any agent capable of facilitating the ligation (i.e., the formation of a phosphodiester bond between a 3′-OH and a 5′ P located at the termini of two strands of nucleic acid). Preferred ligation means comprise DNA ligases and RNA ligases.

The term “reactant” is used herein in its broadest sense. The reactant can comprise, for example, an enzymatic reactant, a chemical reactant or light (e.g., ultraviolet light, particularly short wavelength ultraviolet light is known to break oligonucleotide chains). Any agent capable of reacting with an oligonucleotide to either shorten (i.e., cleave) or elongate the oligonucleotide is encompassed within the term “reactant.”

The term “nucleic acid sequence” as used herein refers to an oligonucleotide, nucleotide or polynucleotide, and fragments or portions thereof, and to DNA or RNA of genomic or synthetic origin that may be single or double stranded, and represent the sense or antisense strand. Similarly, “amino acid sequence” as used herein refers to peptide or protein sequence.

The term “peptide nucleic acid” (“PNA”) as used herein refers to a molecule comprising bases or base analogs such as would be found in natural nucleic acid, but attached to a peptide backbone rather than the sugar-phosphate backbone typical of nucleic acids. The attachment of the bases to the peptide is such as to allow the bases to base pair with complementary bases of nucleic acid in a manner similar to that of an oligonucleotide. These small molecules, also designated anti gene agents, stop transcript elongation by binding to their complementary strand of nucleic acid (Nielsen, et al. Anticancer Drug Des. 8:53 63 [1993]).

As used herein, the terms “purified” or “substantially purified” refer to molecules, either nucleic or amino acid sequences, that are removed from their natural environment, isolated or separated, and are at least 60% free, preferably 75% free, and most preferably 90% free from other components with which they are naturally associated. An “isolated polynucleotide” or “isolated oligonucleotide” is therefore a substantially purified polynucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, allele frequencies, RNA expression levels, protein expression, phenotypes correlating to genotypes, etc. “Allele frequency information” refers to facts or data pertaining allele frequencies, including, but not limited to, allele identities, statistical correlations between the presence of an allele and a characteristic of a subject (e.g., a human subject), the presence or absence of an allele in a individual or population, the percentage likelihood of an allele being present in an individual having one or more particular characteristics, etc.

The term “cleavage structure” as used herein, refers to a structure that is formed by the interaction of at least one probe oligonucleotide and a target nucleic acid, forming a structure comprising a duplex, the resulting structure being cleavable by a cleavage agent, including but not limited to an enzyme. The cleavage structure is a substrate for specific cleavage by the cleavage means in contrast to a nucleic acid molecule that is a substrate for non-specific cleavage by agents such as phosphodiesterases that cleave nucleic acid molecules without regard to secondary structure (i.e., no formation of a duplexed structure is required).

### DESCRIPTION OF THE DRAWINGS

FIG. 1 shows sample embodiments of TAQMAN probes.

FIG. 2 represents one embodiment of the TAQMAN PCR method.

FIG. 3 shows examples of probes labeled with fluorescent dyes.

FIG. 4 shows a sample embodiment of an invasive cleavage structure, e.g., for an INVADER assay.

FIG. 5 shows one embodiment of a FRET probe, e.g., for an INVADER assay.

FIG. 6 shows one embodiment of an INVADER assay.

FIG. 7 shows a diagram of an INVADER assay probe in which the allele does not match the probe.

FIG. 8 shows one embodiment of allele identification using a ligation reaction.

FIG. 9 shows a drawing of the structure of and SNP position in the ATP-binding cassette subfamily B member 2 (ABCB2) gene.

FIG. 10 shows a drawing of the structure of and SNP position in the ATP-binding cassette subfamily B member 4 (ABCB4) gene.

FIG. 11 shows a drawing of the structure of and SNP position in the microsomal epoxide hydrogenase 1 (EPHX1) gene.

FIG. 12 shows a drawing of the structure of and SNP position in the cytoplasmic epoxide hydrogenase 2 (EPHX2) gene.

FIG. 13 shows a drawing of the structure of and SNP position in the guanidinoacetate-N-methyltransferase (GAMT) gene.

FIG. 14 shows a drawing of the structure of and SNP position in the nicotinamide-N-methyltransferase (NNMT) gene.

FIG. 15 shows a drawing of the structure of and SNP position in the phenylethanolamine-N-methyltransferase (PNMT) gene.

FIG. 16 shows a drawing of the structure of and SNP position in the phosphatidylethanolamine-N-methyltransferase (PEMT) gene.

FIG. 17 shows a drawing of the structure of and SNP position in the glutathione-S-methyltransferase 3 (GSTM3) gene.

FIG. 18 shows a drawing of the structure of and SNP position in the aldehyde dehydrogenase 5 (ALDH5) gene.

FIG. 19 shows a drawing of the structure of and SNP position in the transglutaminase (TGM1) gene.

FIG. 20 shows a drawing of the structure of and SNP position in the gamma glutamyltransferase (GGT1) gene.

FIG. 21 shows a drawing of the structure of and SNP position in the NAD(P)H: quinone oxidetransferase (NQ01) gene.

FIG. 22 shows a drawing of the structure of and SNP position in the p53-induced gene 3 (PIG3) of a quinone oxide transferase homologue.

FIG. 23 shows a drawing of the structure of and SNP position in the NRH: quinone oxide transferase 2 (NQ02) gene.

FIG. 24 shows a drawing of the structure of and SNP position in the sulfotransferase 1A1 (SULT1A1/STP1) gene.

FIG. 25 shows a drawing of the structure of and SNP position in the sulfotransferase 1A2 (SULT1A2/STP2) gene.

FIG. 26 shows a drawing of the structure of and SNP position in the sulfotransferase-related protein 3 (SULTX3) gene.

FIG. 27 shows a drawing of the structure of and SNP position in the tyrosyl protein sulfotransferase (TPST1) gene.

FIG. 28 shows a drawing of the structure of and SNP position in the tyrosyl protein sulfotransferase (TPST2) gene.

FIG. 29 shows a drawing of the structure of and SNP position in the sulfotransferase 1A3 (SULT1A3/STM/HAST) gene.

FIG. 30 shows a drawing of the structure of and SNP position in the cerebroside transferase (CST) gene.

FIG. 31 shows a drawing of the structure of and SNP position in the sulfotransferase 1C1 (SULT1C1) gene.

FIG. 32 shows a drawing of the structure of and SNP position in the sulfotransferase 1 C2 (SULT1C2) gene.

FIG. 33 shows a drawing of the structure of and SNP position in the thyroid hormone sulfotransferase (ST1B2) gene.

FIG. 34 shows a drawing of the structure of and SNP position in the hydrocarbon sulfotransferase 2 (CHST2) gene.

FIG. 35 shows a drawing of the structure of and SNP position in the sulfotransferase 2A1 (SULT2A1) gene.

FIG. 36 shows a drawing of the structure of and SNP position in the sulfotransferase 2B1 (SULT2B1) gene.

FIG. 37 shows a drawing of the structure of and SNP position in the hydrocarbon sulfotransferase 4 (CHST4) gene.

FIG. 38 shows a drawing of the structure of and SNP position in the hydrocarbon sulfotransferase 5 (CHST5) gene.

FIG. 39 shows a drawing of the structure of and SNP position in the HNK-sulfotransferase (NHK-1ST) gene.

FIG. 40 shows a drawing of the structure of and SNP position in the estrogen sulfotransferase (STE) gene.

FIG. 41 shows a drawing of the structure of and SNP position in the alcohol dehydrogenase 1 (ADH1) gene.

FIG. 42 shows a drawing of the structure of and SNP position in the alcohol dehydrogenase 2 (ADH2) gene.

FIG. 43 shows a drawing of the structure of and SNP position in the alcohol dehydrogenase 3 (ADH3) gene.

FIG. 44 shows a drawing of the structure of and SNP position in the alcohol dehydrogenase 6 (ADH6) gene.

FIG. 45 shows a drawing of the structure of and SNP position in the alcohol dehydrogenase 7 (ADH7) gene.

FIG. 46 shows a drawing of the structure of and SNP position in the short-chained alcohol dehydrogenase family (HEP27) gene.

FIG. 47 shows a drawing of the structure of and SNP position in the L1 intracellular adhesion molecule (L1CAM) gene.

FIG. 48 shows a drawing of the structure of and SNP position in the arylalkylamine-N-acetyltransferase (AANAT) gene.

FIG. 49 shows a drawing of the structure of and SNP position in the N-actyltransferase homologue (ARD1) gene of Saccharomyces cerevisiae.

FIG. 50 shows a drawing of the structure of and SNP position in the N-actyltransferase 1 (NAT1) gene.

FIG. 51 shows a drawing of the structure of and SNP position in the N-actyltransferase 2 (NAT2) gene.

FIG. 52 shows a drawing of the structure of and SNP position in the granzyme A (GZMA) gene.

FIG. 53 shows a drawing of the structure of and SNP position in the granzyme B (GZMB) gene.

FIG. 54 shows a drawing of the structure of and SNP position in the esterase D-formylglutathione hydrolase (ESD) gene.

FIG. 55 shows a drawing of the structure of and SNP position in the dolichyl-diphosphooligosaccharide-protein glycosyltransferase (DDOST) gene.

FIG. 56 shows a drawing of the structure of and SNP position in the microsomal glutathione-S-transferase (MGST1) gene.

FIG. 57 shows a drawing of the structure of and SNP position in the alcohol dehydrogenase 5 (ADH5) gene.

FIG. 58 shows a drawing of the structure of and SNP position in the glutathione-S-transferase M1 (GSTM1) gene.

FIG. 59 shows a drawing of the structure of and SNP position in the glutathione-S-transferase M2 (GSTM2) gene.

FIG. 60 shows a drawing of the structure of and SNP position in the glutathione-S-transferase M4 (GSTM4) gene.

FIG. 61 shows a drawing of the structure of and SNP position in the glutathione-S-transferase Z1 (GSTZ1) gene.

FIG. 62 shows a drawing of the structure of and SNP position in the glutathione-S-transferase P (GSTZPi) gene.

FIG. 63 shows a drawing of the structure of and SNP position in the glutathione-S-transferase q1 (GSTT1) gene.

FIG. 64 shows a drawing of the structure of and SNP position in the microsomal glutathione-S-transferase 1L1 (MGST1L1) gene.

FIG. 65 shows a drawing of the structure of and SNP position in the microsomal glutathione-S-transferase 2 (MGST2) gene.

FIG. 66 shows a drawing of the structure of and SNP position in the microsomal glutathione-S-transferase 3 (MGST3) gene.

FIG. 67 shows a drawing of the structure of and SNP position in the glutathione-S-transferase A1 (GSTA1) gene.

FIG. 68 shows a drawing of the structure of and SNP position in the glutathione-S-transferase A4 (GSTA4) gene.

FIG. 69 shows a drawing of the structure of and SNP position in the NADH-ubiquinone oxide reductase 1a subcomplex 1 (NDUFA1) gene.

FIG. 70 shows a drawing of the structure of and SNP position in the NADH-ubiquinone oxide reductase 1a subcomplex 2 (NDUFA2) gene.

FIG. 71 shows a drawing of the structure of and SNP position in the NADH-ubiquinone oxide reductase 1a subcomplex 3 (NDUFA3) gene.

FIG. 72 shows a drawing of the structure of and SNP position in the NADH-ubiquinone oxide reductase 1a subcomplex 5 (NDUFA5) gene.

FIG. 73 shows a drawing of the structure of and SNP position in the NADH-ubiquinone oxide reductase 1a subcomplex 6 (NDUFA6) gene.

FIG. 74 shows a drawing of the structure of and SNP position in the NADH-ubiquinone oxide reductase 1a subcomplex 7 (NDUFA7) gene.

FIG. 75 shows a drawing of the structure of and SNP position in the NADH-ubiquinone oxide reductase 1a subcomplex 8 (NDUFA8) gene.

FIG. 76 shows a drawing of the structure of and SNP position in the NADH-ubiquinone oxide reductase 1a/b subcomplex 1 (NDUFAB1) gene.

FIG. 77 shows a drawing of the structure of and SNP position in the NADH-ubiquinone oxide reductase 1a subcomplex 9 (NDUFA9) gene.

FIG. 78 shows a drawing of the structure of and SNP position in the NADH-ubiquinone oxide reductase Fe—S protein 1 (NDUFS1) gene.

FIG. 79 shows a drawing of the structure of and SNP position in the NADH-ubiquinone oxide reductase Fe—S protein 3 (NDUFS3) gene.

FIG. 80 shows a drawing of the structure of and SNP position in the NADH-ubiquinone oxide reductase Fe—S protein 4 (NDUFS4) gene.

FIG. 81 shows a drawing of the structure of and SNP position in the NADH-ubiquinone oxide reductase Fe—S protein 5 (NDUFS5) gene.

FIG. 82 shows a drawing of the structure of and SNP position in the NADH-ubiquinone oxide reductase Fe—S protein 6 (NDUFS6) gene.

FIG. 83 shows a drawing of the structure of and SNP position in the NADH-ubiquinone oxide reductase Fe—S protein 8 (NDUFS8) gene.

FIG. 84 shows a drawing of the structure of and SNP position in the NADH-ubiquinone oxide reductase 1b subcomplex 3 (NDUFB3) gene.

FIG. 85 shows a drawing of the structure of and SNP position in the NADH-ubiquinone oxide reductase 1b subcomplex 5 (NDUFB5) gene.

FIG. 86 shows a drawing of the structure of and SNP position in the NADH-ubiquinone oxide reductase 1b subcomplex 7 (NDUFB7) gene.

FIG. 87 shows a drawing of the structure of and SNP position in the ATP-binding cassette subfamily A member 1 (ABCA1) gene.

FIG. 88 shows a drawing of the structure of and SNP position in the catechol-0-methyltransferase (COMT) gene.

FIG. 89 shows a drawing of the structure of and SNP position in the vitamin-N-transferase (HNMT) gene.

FIG. 90 shows a drawing of the structure of and SNP position in the cytochrome P450 subfamily 1 (aromatic compound-induced) polypeptide 1 (CYP1A1) gene.

FIG. 91 shows a drawing of the structure of and SNP position in the cytochrome P450 subfamily 1 (aromatic compound-induced) polypeptide 2 (CYP1A2) gene.

FIG. 92 shows a drawing of the structure of and SNP position in the cytochrome P450 subfamily 1 (dioxin-induced) polypeptide 1 (CYP1B1) gene.

FIG. 93 shows a drawing of the structure of and SNP position in the arylacetamide deactylase (AADAC) gene.

FIG. 94 shows a drawing of the structure of and SNP position in the neuropathy target esterase (NTE) gene.

FIG. 95 shows a drawing of the structure of and SNP position in the ATP-binding cassette subfamily C (CFTR/MRP) member 2 (MRP2) gene.

FIG. 96 shows a drawing of the structure of and SNP position in the ATP-binding cassette subfamily B member 1 (ABCB1) gene.

FIG. 97 shows a drawing of the structure of and SNP position in the ATP-binding cassette subfamily B member 3 (ABCB3) gene.

FIG. 98 shows a drawing of the structure of and SNP position in the ATP-binding cassette subfamily B member 7 (ABCB7) gene.

FIG. 99 shows a drawing of the structure of and SNP position in the ATP-binding cassette subfamily B member 8 (ABCB8) gene.

FIG. 100 shows a drawing of the structure of and SNP position in the ATP-binding cassette subfamily B member 9 (ABCB9) gene.

FIG. 101 shows a drawing of the structure of and SNP position in the ATP-binding cassette subfamily B member 10 (ABCB10) gene.

FIG. 102 shows a drawing of the structure of and SNP position in the ATP-binding cassette subfamily B member 11 (ABCB11) gene.

FIG. 103 shows a drawing of the structure of and SNP position in the cytochrome P450 subfamily IVB polypeptide 1 (CYP4B1) gene.

FIG. 104 shows a drawing of the structure of and SNP position in the cytochrome P450 subfamily XXVIIA polypeptide 1 (CYP27A1) gene.

FIG. 105 shows a drawing of the structure of and SNP position in the cytochrome P450 subfamily IVF polypeptide 1 (CYP4F2) gene.

FIG. 106 shows a drawing of the structure of and SNP position in the cytochrome P450 subfamily 4F polypeptide 3 (CYP4F3) gene.

FIG. 107 shows a drawing of the structure of and SNP position in the cytochrome P450 subfamily 4F polypeptide 8 (CYP4F8) gene.

FIG. 108 shows a drawing of the structure of and SNP position in the aldehyde dehydrogenase 1 (ALDH1) gene.

FIG. 109 shows a drawing of the structure of and SNP position in the aldehyde dehydrogenase 2 (ALDH2) gene.

FIG. 110 shows a drawing of the structure of and SNP position in the aldehyde dehydrogenase 7 (ALDH7) gene.

FIG. 111 shows a drawing of the structure of and SNP position in the aldehyde dehydrogenase 8 (ALDH8) gene.

FIG. 112 shows a drawing of the structure of and SNP position in the aldehyde dehydrogenase 9 (ALDH9) gene.

FIG. 113 shows a drawing of the structure of and SNP position in the aldehyde dehydrogenase 10 (ALDH10) gene.

FIG. 114 shows a drawing of the structure of and SNP position in the ATP-binding cassette subfamily C member 7 (ABCC7) gene.

FIG. 115 shows a drawing of the structure of and SNP position in the ATP-binding cassette subfamily C member 8 (ABCC8) gene.

FIG. 116 shows a drawing of the structure of and SNP position in the ATP-binding cassette subfamily C member 9 (ABCC9) gene.

FIG. 117 shows a drawing of the structure of and SNP position in the carboxylesterase 1 (CES1) gene.

FIG. 118 shows a drawing of the structure of and SNP position in the ATP-binding cassette subfamily A member 4 (AB(CC4) gene.

FIG. 119 shows a drawing of the structure of and SNP position in the ATP-binding cassette subfamily A member 7 (ABCC7) gene.

FIG. 120 shows a drawing of the structure of and SNP position in the ATP-binding cassette subfamily G member 1 (ABCG1) gene.

FIG. 121 shows a drawing of the structure of and SNP position in the ATP-binding cassette subfamily G member 2 (ABCG2) gene.

FIG. 122 shows a drawing of the structure of and SNP position in the ATP-binding cassette subfamily G member 4 (ABCG4) gene.

FIG. 123 shows a drawing of the structure of and SNP position in the ATP-binding cassette subfamily E member 1 (ABCE1) gene.

FIG. 124 shows a drawing of the structure of and SNP position in the carbohydrate sulfotransferase 1 (CHST1) gene.

FIG. 125 shows a drawing of the structure of and SNP position in the carbohydrate sulfotransferase 3 (CHST3) gene.

FIG. 126 shows a drawing of the structure of and SNP position in the NADH: ubiquinone dehydrogenase flavoprotein 1 (NDUFV1) gene.

FIG. 127 shows a drawing of the structure of and SNP position in the NADH: ubiquinone dehydrogenase flavoprotein 2 (NDUFV2) gene.

FIG. 128 shows a drawing of the structure of and SNP position in the NADH: ubiquinone dehydrogenase flavoprotein 3 (NDUFV3) gene.

FIG. 129 shows a drawing of the structure of and SNP position in the NADH: ubiquinone oxide reductase A10 (NDUFA10) gene.

FIG. 130 shows a drawing of the structure of and SNP position in the high-mobility group protein 17-like 1 (HMG17L1) gene.

FIG. 131 shows a drawing of the structure of and SNP position in the UDP glycoxyl transferase 2 family polypeptide A1 (UGT2A1) gene.

FIG. 132 shows a drawing of the structure of and SNP position in the human organic anion transporter polypeptide 1 (hOATP1) gene.

FIG. 133 shows a drawing of the structure of and SNP position in the human organic anion transporter polypeptide 2 (hOATP2) gene.

FIG. 134 shows a drawing of the structure of and SNP position in the human organic anion transporter polypeptide 8 (hOATP8) gene.

FIG. 135 shows a drawing of the structure of and SNP position in the human organic anion transporter 1 (hOAT1) gene.

FIG. 136 shows a drawing of the structure of and SNP position in the human organic anion transporter 2 (hOAT2) gene.

FIG. 137 shows a drawing of the structure of and SNP position in the human organic anion transporter 3 (hOAT3) gene.

FIG. 138 shows a drawing of the structure of and SNP position in the aldehyde dehydrogenase 1 family member A2 (ALDH1A2) gene.

FIG. 139 shows a drawing of the structure of and SNP position in the aldehyde dehydrogenase 1 family member A3 (ALDH1A3) gene.

FIG. 140 shows a drawing of the structure of and SNP position in the formyltetrahydroforate dehydrogenase (FTHFD/ALDH1L1) gene.

FIG. 141 shows a drawing of the structure of and SNP position in the cytochrome P450 subfamily IIIA (aromatic compound-induced) polypeptide 4 (CYP3A4) gene.

FIG. 142 shows graph of the results of typing performed on two different groups of subjects using the INVADER assay method.

FIG. 143 shows a summary of genetic information.

FIG. 144A shows a structure of ATP-binding cassette subfamily A member 1 (ABCA1) gene and the SNP location therein.

Accession No.: AF275948.1 and AL359846.11

FIG. 144B shows a structure of ATP-binding cassette subfamily A member 1 (ABCA1) gene and the SNP location therein. (continuation of FIG. 144A)

Accession No.: AF275948.1 and AL359846.11

FIG. 145 shows a structure of ATP-binding cassette subfamily A member 4 (ABCA4) gene and the SNP location therein.

Accession No.: NT_{—}019258.1

FIG. 146 shows a structure of ATP-binding cassette subfamily A member 7 (ABCA7) gene and the SNP location therein.

Accession No.: NT_{—}025194.1

FIG. 147 shows a structure of ATP-binding cassette subfamily A member 8 (ABCA8) gene and the SNP location therein.

Accession No.: AC005922.1 and AC015844.5

FIG. 148 shows a structure of ATP-binding cassette subfamily B member 1 (ABCB1) gene and the SNP location therein.

Accession No.: AC002457.1 and AC005068.1

FIG. 149 shows a structure of ATP-binding cassette subfamily B member 4 (ABCB4) gene and the SNP location therein.

Accession No.: AC079591.1, AC079303.3 and AC005045.2

FIG. 150 shows a structure of ATP-binding cassette subfamily B member 7 (ABCB7) gene and the SNP location therein.

Accession No.: AL360179.3 and AC002417.1

FIG. 151 shows a structure of ATP-binding cassette subfamily B member 8 (ABCB8) gene and the SNP location therein.

Accession No.: AC010973.4

FIG. 152 shows a structure of ATP-binding cassette subfamily B member 9 (ABCB9) gene and the SNP location therein.

Accession No.: AC026362.9 and AC073857.10

FIG. 153 shows a structure of ATP-binding cassette subfamily B member 10 (ABCB10) gene and the SNP location therein.

Accession No.: AL121990.9

FIG. 154 shows a structure of ATP-binding cassette subfamily B member 11 (ABCB11) gene and the SNP location therein.

Accession No.: AC008177.3 and AC069137.3

FIG. 155 shows a structure of ATP-binding cassette subfamily C member 1 (ABCC1) gene and the SNP location therein.

Accession No.: AC026452.5 and AC025778.4

FIG. 156 shows a structure of ATP-binding cassette subfamily C member 2 (ABCC2) gene and the SNP location therein.

Accession No.: AL392107.4

FIG. 157 shows a structure of ATP-binding cassette subfamily C member 3 (ABCC3) gene and the SNP location therein.

Accession No.: AC004590.1 and AC005921.3

FIG. 158A shows a structure of ATP-binding cassette subfamily C member 4 (ABCC4) gene and the SNP location therein.

Accession No.: AL356257.11, AL157818.12 and AL139381.12

FIG. 158B shows a structure of ATP-binding cassette subfamily C member 4 (ABCC4) gene and the SNP location therein. (continuation of FIG. 158A)

Accession No.: AL356257.11, AL15,7818.12, and AL139381.12

FIG. 159 shows a structure of ATP-binding cassette subfamily C member 5 (ABCC5) gene and the SNP location therein.

Accession No.: AC068644.5

FIG. 160 shows a structure of ATP-binding cassette subfamily C member 7 (ABCC7) gene and the SNP location therein.

Accession No.: AC000111.1 and AC000061.1

FIG. 161 shows a structure of ATP-binding cassette subfamily C member 8 (ABCC8) gene and the SNP location therein.

Accession No.: AC000406.1

FIG. 162 shows a structure of ATP-binding cassette subfamily C member 9 (ABCC9) gene and the SNP location therein.

Accession No.: AC084806.9 and AC008250.23

FIG. 163 shows a structure of ATP-binding cassette subfamily D member 1 (ABCD1) gene and the SNP location therein.

Accession No.: U52111.2

FIG. 164 shows a structure of ATP-binding cassette subfamily D member 3 (ABCD3) gene and the SNP location therein.

Accession No.: NT_{—}019284.3

FIG. 165 shows a structure of ATP-binding cassette subfamily D member 4 (ABCD4) gene and the SNP location therein.

Accession No.: AC005519.3

FIG. 166 shows a structure of ATP-binding cassette subfamily G member 1 (ABCG1) gene and the SNP location therein.

Accession No.: AP001746.1

FIG. 167 shows a structure of ATP-binding cassette subfamily G member 2 (ABCG2) gene and the SNP location therein.

Accession No.: NT_{—}022959.2

FIG. 168 shows a structure of ATP-binding cassette subfamily G member 4 (ABCG4) gene and the SNP location therein.

Accession No.: AP001315.3

FIG. 169 shows a structure of ATP-binding cassette subfamily G member 5 (ABCG5) gene and the SNP location therein.

Accession No.: AC084265.2 and AC011242.8

FIG. 170 shows a structure of ATP-binding cassette subfamily G member 8 (ABCG8) gene and the SNP location therein.

Accession No.: AC084265.2

FIG. 171 shows a structure of ATP-binding cassette subfamily E member 1 (ABCE1) gene and the SNP location therein.

Accession No.: NT_{—}006296.2

FIG. 172 shows a structure of ATP -binding cassette subfamily F member 1 (ABCF1) gene and the SNP location therein.

Accession No.: NT_{—}007592.3

FIG. 173 shows a structure of organic anion transporter 1 (OAT1) gene and the SNP location therein.

Accession No.: AP001858.3, AJ249369.1, and AP000438.4

FIG. 174 shows a structure of organic anion transporter 2 (OAT2) gene and the SNP location therein.

Accession No.: AC26532.3

FIG. 175 shows a structure of organic anion transporter 3 (OAT3) gene and the SNP location therein.

Accession No.: AP001858.3

FIG. 176 shows a structure of organic anion transporter polypeptide 1 (OATP1) gene and the SNP location therein.

Accession No.: AC022224.22

FIG. 177 shows a structure of organic anion transporter polypeptide 2 (OATP2) gene and the SNP location therein.

Accession No.: NT_{—}024399.2

FIG. 178 shows a structure of organic anion transporter polypeptide 8 (OATP8) gene and the SNP location therein.

Accession No.: NT_{—}024399.2

FIG. 179 shows a structure of transporter 1 ATP-binding cassette subfamily B (TAP1) gene and the SNP location therein.

Accession No.: X66401.1

FIG. 180 shows a structure of transporter 2 ATP-binding cassette subfamily B (TAP2) gene and the SNP location therein.

Accession No.: X6640 1.1

FIG. 181 shows a structure of SLC22A4 solute carrier family 22 (organic cation transporter) member 4 (OCTN1) gene and the SNP location therein.

Accession No.: AC008599.6

FIG. 182 shows a structure of SLC22A5 solute carrier family 22 (organic cation transporter) member 5 (OCTN2) gene and the SNP location therein.

Accession No.: AC023861.3

FIG. 183 shows a structure of SLC22A1 solute carrier family 22 (organic cation transporter) member 1 (OCT1) gene and the SNP location therein.

Accession No.: AL35625.5

FIG. 184 shows a structure of SLC22A2 solute carrier family 22 (organic cation transporter) member 2 (OCT2) gene and the SNP location therein.

Accession No.: AL162582.18

FIG. 185 shows a structure of SLC10A2 solute carrier family 10 (sodium/bile acid cotransporter family) member 2 (NTCP) gene and the SNP location therein.

Accession No.: AL157789.6

FIG. 186 shows a structure of SLC15A1 solute carrier family 15 (oligopeptide transporter) member 1 (PEPT1) gene and the SNP location therein.

Accession No.: AL353574.8 and AL391670.6

FIG. 187 shows a structure of microsomal epoxide hydrolase 1 (EPHX1) gene and the SNP location therein.

Accession No.: AC058782.8

FIG. 188 shows a structure of cytoplasmic epoxide hydrolase (EPHX2) gene and the SNP location therein.

Accession No.: AC010856.3

FIG. 189 shows a structure of catechol-O-methyl transferase (COMT) gene and the SNP location therein.

Accession No.: AC000080.2

FIG. 190 shows a structure of guanidinoacetate N-methyl transferase (GAMT) gene and the SNP location therein.

Accession No.: NT_{—}000879.1

FIG. 191 shows a structure of phenyl ethanolamine N-methyl transferase (PNMT) gene and the SNP location therein.

Accession No.: AC040933.3

FIG. 192 shows a structure of histamine N-methyl transferase (HNMT) gene and the SNP location therein.

Accession No.: AC019304.3

FIG. 193 shows a structure of nicotinamide N-methyl transferase (NNMT) gene and the SNP location therein.

Accession No.: AC019290.3

FIG. 194 shows a structure of phosphatidylethanolamine N-methyl transferase (PEMT) gene and the SNP location therein.

Accession No.: AC020558.3

FIG. 195 shows a structure of aldehyde dehydrogenase 1 family member A1 (ALDH1A1) gene and the SNP location therein.

Accession No.: AC009284.2 and AL162416.3

FIG. 196 shows a structure of aldehyde dehydrogenase 1 family member A2 (ALDH1A2) gene and the SNP location therein.

Accession No.: AC025431.7 and AC012653.8

FIG. 197 shows a structure of aldehyde dehydrogenase 1 family member A3 (ALDH1A3) gene and the SNP location therein.

Accession No.: AC015712.7

FIG. 198 shows a structure of aldehyde dehydrogenase 1 family member B1 (ALDH1B1) gene and the SNP location therein.

Accession No.: AL135785.9

FIG. 199A shows a structure of formyl tetrahydrofolate dehydrogenase (ALDH1L1) gene and the SNP location therein.

Accession No.: AC079848.6

FIG. 199B shows a structure of fornyl tetrahydrofolate dehydrogenase (ALDH1L1) gene and the SNP location therein. (continuation of FIG. 199A)

Accession No.: AC079848.6

FIG. 200 shows a structure of aldehyde dehydrogenase 2 (ALDH2) gene and the SNP location therein.

Accession No.: AC002996.1 and AC003029.2

FIG. 201 shows a structure of aldehyde dehydrogenase 3 family member A1 (ALDH3A1) gene and the SNP location therein.

Accession No.: AC005722.1

FIG. 202 shows a structure of aldehyde dehydrogenase 3 family member A2 (ALDH3A2) gene and the SNP location therein.

Accession No.: AC005722.1

FIG. 203 shows a structure of aldehyde dehydrogenase 3 family member B1 (ALDH3B1) gene and the SNP location therein.

Accession No.: AC004923.2

FIG. 204 shows a structure of aldehyde dehydrogenase 3 family member B2 (ALDH3B2) gene and the SNP location therein.

Accession No.: AC021987.3

FIG. 205 shows a structure of aldehyde dehydrogenase 5 family member A1 (ALDH5A1) gene and the SNP location therein.

Accession No.: AL031230.1

FIG. 206 shows a structure of aldehyde dehydrogenase 6 family member A1 (ALDH6A1) gene and the SNP location therein.

Accession No.: AC005484.2

FIG. 207 shows a structure of aldehyde dehydrogenase 8 family member A1 (ALDH8A1) gene and the SNP location therein.

Accession No.: AL445190.9 and AL021939.1

FIG. 208 shows a structure of aldehyde dehydrogenase 9 family member A1 (ALDH9A1) gene and the SNP location therein.

Accession No.: AL451074.4

FIG. 209 shows a structure of alcohol dehydrogenase 1 (ADH1) gene and the SNP location therein.

Accession No.: AP002027.1

FIG. 210 shows a structure of alcohol dehydrogenase 2 (ADH2) gene and the SNP location therein.

Accession No.: AP002027.1

FIG. 211 shows a structure of alcohol dehydrogenase 3 (ADH3) gene and the SNP location therein.

Accession No.: AP002027.1

FIG. 212 shows a structure of alcohol dehydrogenase 4 (ADH4) gene and the SNP location therein.

Accession No.: AP002026.1

FIG. 213 shows a structure of alcohol dehydrogenase 5 (ADH5) gene and the SNP location therein.

Accession No.: AC019131.4

FIG. 214 shows a structure of alcohol dehydrogenase 6 (ADH6) gene and the SNP location therein.

Accession No.: AP002026.1

FIG. 215 shows a structure of alcohol dehydrogenase 7 (ADH7) gene and the SNP location therein.

Accession No.: AC027065.3

FIG. 216 shows a structure of short-chain alcohol dehydrogenase family gene (HEP27) and the SNP location therein.

Accession No.: AL135999.3

FIG. 217 shows a structure of UDP glycosyltransferase 1 family polypeptide A1 (UGT1A1) and the SNP location therein.

Accession No.: AC006985.2

FIG. 218 shows a structure of UDP glycosyltransferase 2 family polypeptide A1 (UGT2A1) and the SNP location therein.

Accession No.: AC011254.3

FIG. 219 shows a structure of UDP glycosyltransferase 2 family polypeptide B15 (UGT2B15) and the SNP location therein.

Accession No.: AC019173.4

FIG. 220 shows a structure of UDP glycosyltransferase 8 (UGT8) and the SNP location therein.

Accession No.: U31353.1

FIG. 221 shows a structure of glutathione S transferase A1 (GSTA1) gene and the SNP location therein.

Accession No.: AC021133.4

FIG. 222 shows a structure of glutathione S transferase A4 (GSTA4) gene and the SNP location therein.

Accession No.: AC025085.4

FIG. 223 shows a structure of glutathione S transferase M1 (GSTM1) gene and the SNP location therein.

Accession No.: AC000032.7

FIG. 224 shows a structure of glutathione S transferase M2 (GSTM2) gene and the SNP location therein.

Accession No.: AC000031.5

FIG. 225 shows a structure of glutathione S transferase Z1 (GSTZ1) gene and the SNP location therein.

Accession No.: AC007954.7

FIG. 226 shows a structure of glutathione S transferase Pi (GSTPi) gene and the SNP location therein.

Accession No.: X08058.1 and M24485.1

FIG. 227 shows a structure of glutathione S transferase T1 (GSTT1) gene and the SNP location therein.

Accession No.: AF240786.1 and AP000351.3

FIG. 228 shows a structure of microsomal glutathione S transferase 1 (MGST1) gene and the SNP location therein.

Accession No.: AC007528.5

FIG. 229 shows a structure of microsomal glutathione S transferase 1-like 1 (MGST1L1) gene and the SNP location therein.

Accession No.: AC007936.2

FIG. 230 shows a structure of microsomal glutathione S transferase T2 (MGST2) gene and the SNP location therein.

Accession No.: AC019049.4

FIG. 231 shows a structure of microsomal glutathione S transferase T3 (MGST3) gene and the SNP location therein.

Accession No.: AC064827.2

FIG. 232 shows a structure of sulfotransferase 1A1 (SULT1A1/STP1) gene and the SNP location therein.

Accession No.: U52852.2

FIG. 233 shows a structure of sulfotransferase 1A2 (SULT1A2/STP2) gene and the SNP location therein.

Accession No.: U33886.1, U34804.1 and AC020765.5

FIG. 234 shows a structure of sulfctransferase 1A3 (SULT1A3/STM/HAST) gene and the SNP location therein

Accession No.: L34160.1 and AC012645.4

FIG. 235 shows a structure of sulfotransferase 1C1 (SULT1C1) gene and the SNP location therein.

Accession No.: AC019100.4

FIG. 236 shows a structure of sulfotransferase 1C2 (SULT1C2) gene and the SNP location therein.

Accession No.: AF186263.1

FIG. 237 shows a structure of sulfotransferase 2A1 (SULT2A1) gene and the SNP location therein.

Accession No.: AC024582.4, AC008745.5, NT011190.1, and AC024582.4

FIG. 238 shows a structure of sulfotransferase 2B1 (SULT2B1) gene and the SNP location therein.

Accession No.: AC040922.2 and AC008403.6

FIG. 239 shows a structure of sulfotransferase-associated protein 3 (SULTX3) gene and the SNP location therein.

Accession No.: Z97055.1

FIG. 240 shows a structure of tyrosyl protein sulfotransferase 1 (TPST1) gene and the SNP location therein.

Accession No.: AC026281.5

FIG. 241 shows a structure of tyrosyl protein sulfotransferase 2 (TPST2) gene and the SNP location therein.

Accession No.: Z95115.1

FIG. 242 shows a structure of cerebroside sulfotransferase (CST) gene and the SNP location therein.

Accession No.: AC005006.2

FIG. 243 shows a structure of thyroid hormone sulfotransferase (ST1B2) gene and the SNP location therein.

Accession No.: AC027059.2

FIG. 244 shows a structure of carbohydorate sulfotransferase 1 (CHST1) gene and the SNP location therein.

Accession No.: NT_{—}008982.1

FIG. 245 shows a structure of carbohydorate sulfotransferase 2 (CHST2) gene and the SNP location therein.

Accession No.: AC055737.10

FIG. 246 shows a structure of carbohydorate sulfotransferase 3 (CHST3) gene and the SNP location therein.

Accession No.: AC073370.3

FIG. 247 shows a structure of carbohydorate sulfotransferase 4 (CHST4) gene and the SNP location therein.

Accession No.: AC010547.5

FIG. 248 shows a structure of carbohydorate sulfotransferase 5 (CHST5) gene and the SNP location therein.

Accession No.: AC025287.3

FIG. 249 shows a structure of HNK-sulfotransferase (HNK-1ST) gene and the SNP location therein.

Accession No.: AC012493.4

FIG. 250 shows a structure of estrogen sulfotransferase (STE) gene and the SNP location therein.

Accession No.: AC074273.1

FIG. 251 shows a structure of NAD (P)H: quinone oxidoreductase 1 (NQO1) gene and the SNP location therein.

Accession No.: M81596.1

FIG. 252 shows a structure of NRH: quinone oxidoreductase 2 (NQO2) gene and the SNP location therein.

Accession No.: AB050248.1

FIG. 253 shows a structure of p53-inducible gene 3 (PIG3) in a quinone oxidoreductase homolog and the SNP location therein.

Accession No.: AC008073.3

FIG. 254 shows a structure of NADH-dehydrogenase(ubiquinone) 1α-subcomplex 1 (NDUFA1) gene and the SNP location therein.

Accession No.: AC002477.1

FIG. 255 shows a structure of NADH-dehydrogenase(ubiquinone) 1α-subcomplex 2 (NDUFA2) gene and the SNP location therein.

Accession No.: AB054976.1

FIG. 256 shows a structure of NADH-dehydrogenase(ubiquinone) 1α-subcomplex 3 (NDUFA3) gene and the SNP location therein.

Accession No.: AC009968.6

FIG. 257 shows a structure of NADH-dehydrogenase(ubiquinone) 1α-subcomplex 5 (NDUFA5) gene and the SNP location therein.

Accession No.: AC073323.5

FIG. 258 shows a structure of NADH-dehydrogenase(ubiquinone) 1α-subcomplex 6 (NDUFA6) gene and the SNP location therein.

Accession No.: AL021878.1

FIG. 259 shows a structure of NADH-dehydrogenase(ubiquinone) 1α-subcomplex 7 (NDUFA7) gene and the SNP location therein.

Accession No.: AC010323.6

FIG. 260 shows a structure of NADH-dehydrogenase(ubiquinone) 1α-subcomplex 8 (NDUFA8) gene and the SNP location therein.

Accession No.: AL162423.10

FIG. 261 shows a structure of NADH-dehydrogenase(ubiquinone) 1α-subcomplex 9 (NDUFA9) gene and the SNP location therein.

Accession No.: AC005832.1

FIG. 262 shows a structure of NADH-dehydrogenase(ubiquinone) 1α-subcomplex 10 (NDUFA10) gene and the SNP location therein.

Accession No.: AC013469.8

FIG. 263 shows a structure of NADH-dehydrogenase(ubiquinone) 1α/β-subcomplex 1 (NDUFAB1) gene and the SNP location therein.

Accession No.: AC008870.6

FIG. 264 shows a structure of NADH-dehydrogenase(ubiquinone) 1β-subcomplex 3 (NDUFB3) gene and the SNP location therein.

Accession No.: AC007272.3

FIG. 265 shows a structure of NADH-dehydrogenase(ubiquinone) 1β-subcomplex 5 (NDUFB5) gene and the SNP location therein.

Accession No.: AC068361.2

FIG. 266 shows a structure of NADH-dehydrogenase(ubiquinone) 1β-subcomplex 7 (NDUFB7) gene and the SNP location therein.

Accession No.: AC010527.4

FIG. 267 shows a structure of NADH-dehydrogenase(ubiquinone) Fe—S protein 1 (NDUFS1) gene and the SNP location therein.

Accession No.: AC007383.4

FIG. 268 shows a structure of NADH-dehydrogenase(ubiquinone) Fe—S protein 3 (NDUFS3) gene and the SNP location therein.

Accession No.: AC067943.4

FIG. 269 shows a structure of NADH-dehydrogenase(ubiquinone) Fe—S protein 4 (NDUFS4) gene and the SNP location therein.

Accession No.: AC024569.3

FIG. 270 shows a structure of NADH-dehydrogenase(ubiquinone) Fe—S protein 5 (NDUFS5) gene and the SNP location therein.

Accession No.: AL139015.5

FIG. 271 shows a structure of NADH-dehydrogenase(ubiquinone) Fe—S protein 6 (NDUFS6) gene and the SNP location therein.

Accession No.: AC026443.2

FIG. 272 shows a structure of NADH-dehydrogenase(ubiquinone) Fe—S protein 8 (NDUFS8) gene and the SNP location therein.

Accession No.: AC034259.2

FIG. 273 shows a structure of NADH-dehydrogenase(ubiquinone) flavoprotein 1 (NDUFV1) gene and the SNP location therein.

Accession No.: NT_{—}009304.2

FIG. 274 shows a structure of NADH-dehydrogenase(ubiquinone) flavoprotein 2 (NDUFV2) gene and the SNP location therein.

Accession No.: NT_{—}011024.2

FIG. 275 shows a structure of NADH-dehydrogenase(ubiquinone) flavoprotein 3 (NDUFV3) gene and the SNP location therein.

Accession No.: AP001748.1

FIG. 276 shows a structure of gamma-glutamyl transferase 1 (GGT1) gene and the SNP location therein.

Accession No.: D87002.1

FIG. 277 shows a structure of transglutaminase 1 (TGM1) gene and the SNP location therein.

Accession No.: M98447.1

FIG. 278 shows a structure of cytochrome P450 subfamily 1 (aromatic compound-inducible) polypeptide 1 (CYP1A1) gene and the SNP location therein.

Accession No.: X04300.1 and AC020705.4

FIG. 279 shows a structure of cytochrome P450 subfamily 1 (aromatic compound-inducible) polypeptide 2 (CYP1A2) gene and the SNP location therein.

Accession No.: AC020705.4

FIG. 280 shows a structure of cytochrome P450 subfamily 1 (dioxin-inducible) polypeptide 1 (CYP1B1) gene and the SNP location therein.

Accession No.: AC009229.4

FIG. 281 shows a structure of cytochrome P450 subfamily 3A (aromatic compound-inducible) polypeptide 4 (CYP3A4) gene and the SNP location therein.

Accession No.: AF280107.1

FIG. 282 shows a structure of cytochrome P450 subfamily 3A (aromatic compound-inducible) polypeptide 5 (CYP3A5) gene and the SNP location therein.

Accession No.: AC005020.5

FIG. 283 shows a structure of cytochrome P450 subfamily 3A polypeptide 7 (CYP3A7) gene and the SNP location therein.

Accession No.: AF280107.1

FIG. 284 shows a structure of cytochrome P450 polypeptide 43 (CYP3A43) gene and the SNP location therein.

Accession No.: AC011904.3

FIG. 285 shows a structure of cytochrome P450 subfamily IVB polypeptide 1 (CYP4B1) gene and the SNP location therein.

Accession No.: AL356793.10

FIG. 286 shows a structure of cytochrome P450 subfamily IVF polypeptide 2 (CYP4F2) gene and the SNP location therein.

Accession No.: AC005336.1

FIG. 287 shows a structure of cytochrome P450 subfamily IVF polypeptide 3 (CYP4F3) gene and the SNP location therein.

Accession No.: AD000685. 1

FIG. 288 shows a structure of cytochrome P450 subfamily IVF polypeptide 8 (CYP4F8) gene and the SNP location therein.

Accession No.: AC068845.3

FIG. 289 shows a structure of cytochrome P450 subfamily XXVIIA polypeptide 1 (CYP27A1) gene and the SNP location therein.

Accession No.: AC009974.7

FIG. 290 shows a structure of cytochrome P450 subfamily XXVIIB polypeptide 1 (CYP27B1) gene and the SNP location therein.

Accession No.: AC025165.27

FIG. 291 shows a structure of allylacetamide deacetylase (AADAC) gene and the SNP location therein.

Accession No.: AC068647.4

FIG. 292 shows a structure of carboxyl esterase 1 (CES1) gene and the SNP location therein

Accession No.: AC007602.4

FIG. 293 shows a structure of carboxyl esterase 2 (CES2) gene and the SNP location therein

Accession No.: AC027131.4

FIG. 294 shows a structure of granzyme A (GZMA) gene and the SNP location therein.

Accession No.: AC091977.1

FIG. 295 shows a structure of granzyme B (GZMB) gene and the SNP location therein.

Accession No.: AL136018.3

FIG. 296 shows a structure of esterase D/formylglutathione hydrolase (ESD) gene and the SNP location therein.

Accession No.: AL136958.9

FIG. 297A shows a structure of carboxyl ester lipase (bile salt-stimulated lipase) (CEL) gene and the SNP location therein.

Accession No.: AL138750.8, AL162417.20 and AF072711.1

FIG. 297B shows a structure of carboxyl ester lipase (bile salt-stimulated lipase) (CEL) gene and the SNP location therein. (continuation of FIG. 297A)

Accession No.: AL138750., AL162417.20 and AF072711.1

FIG. 298 shows a structure of interLeukin 17 (cytotoxic T lymphocyte-associated serine esterase 8) (IL17) gene and the SNP location therein.

Accession No.: AL355513.11

FIG. 299 shows a structure of ubiquitin carboxyl terminal esterase L3 (ubiquitin thiol esterase) (UCHL3) gene and the SNP location therein.

Accession No.: AL137244.28

FIG. 300 shows a structure of dolichyl-diphosphooligosaccharide-protein glycosyltransferase (DDOST) gene and the SNP location therein.

Accession No.: D89060

FIG. 301 shows a structure of neuropathy target esterase (NTE) gene and the SNP location therein.

Accession No.: AC021153

FIG. 302 shows a structure of L1 cell adhesion molecule (L1CAM) gene and the SNP location therein.

Accession No.: U52112

FIG. 303 shows a structure of arylalkylamine N-acetyltransferase (AANAT) gene and the SNP location therein.

Accession No.: U40391

FIG. 304 shows a structure of N-acetyltransferase homolog (ARD1) gene of Saccharomyces cerevisiae and the SNP location therein.

Accession No.: U52112

FIG. 305 shows a structure of N-acetyltransferase (NAT1) gene and the SNP location therein.

Accession No.: X17059

FIG. 306 shows a structure of N-acetyltransferase 2 (NAT2) gene and the SNP location therein.

Accession No.: D10870

FIG. 307 shows a structure of ATP-binding cassette subfamily B member 2 (ABCB2) gene and the SNP location therein.

Accession No.: X66401

FIG. 308 shows a structure of ATP-binding cassette subfamily B member 3 (ABCB3) gene and the SNP location therein.

Accession No.: X66401

FIG. 309 shows a structure of glutathione S transferase M3 (GSTM3) gene and the SNP location therein.

Accession No.: AF043105.1

FIG. 310 shows a structure of glutathione S transferase M4 (GSTM4) gene and the SNP location therein.

Accession No.: M96233.1

FIG. 311 shows a structure of aldehyde dehydrogenase 7 (ALDH7) gene and the SNP location therein.

Accession No.: AC004923

FIG. 312 shows a structure of high-mobility group protein 17-like 1 (HMG17L1) gene and the SNP location therein.

Accession No.: Z97055.1

FIG. 313 shows a printed representation of submissions from Laboratory for Genotyping, The SNP Research Center, The Institute of Physical and Chemical Research (RIKEN) on the IMS-JST JSNP database website

### GENERAL DESCRIPTION OF THE INVENTION

The present invention provides a method of analysis of drug metabolizing enzymes by analysis of SNPs associated with their encoding genes. In some embodiments, the method of the present invention can be used in the selection of drugs based on, e.g., particular characteristics of an individual patient or on characteristics of a target disease.

In some embodiments, the present invention provides a method for detecting a genetic polymorphism associated with a DME, wherein an oligonucleotide probe and/or oligonucleotide primer is created so as to include the genetic polymorphism site from genetic polymorphism data in a gene for encoding a drug metabolizing enzyme or so as to include the genetic polymorphism site in an amplified fragment when the gene encoding the drug metabolizing enzyme has been amplified, and wherein at least one genetic polymorphism in a gene for encoding the target drug metabolizing enzyme is detected using the oligonucleotide probe and/or oligonucleotide primer thus obtained.

The present invention further provides methods for evaluating a drug, wherein the effectiveness and safety of a drug metabolized by the drug metabolizing enzyme are evaluated based on the results obtained by the detection method.

In some embodiments, the present invention provides a method for screening a drug, wherein the drug to be used is selected based on the results obtained in the evaluation method. In other embodiments, the present invention provides a method for screening a drug, wherein the genetic polymorphism data associated with the gene encoding a DME in a control subject is compared to the genetic polymorphism data associated with the same gene in a test subject, and wherein a drug to be used is selected from the results of an analysis of the effectiveness and/or safety of the drugs metabolized by the drug metabolizing enzyme.

TABLE 1

| | | | | |
|---|---|---|---|---|
| | | | | SEQ |
| | | | | ID |
| GENE | NO | LOCATION | SEQ. | NO. |

| | | | | | | |
|---|---|---|---|---|---|---|
| ABCB2 | 1 | 5′flanking − 673 | agctaagagtcaaagcaccc G/C ctttttccaccagcctcgcg | 1 | | |
| ABCB2 | 2 | 5′flanking − 646 | ccaccagcctcgcgtgcctg T/G tcccttcacggacactctag | 2 | | |
| ABCB2 | 3 | 5′flanking − 563 | ttgcaagcgctggctgctac A/C ggcgacctccctgcgctccc | 3 | | |
| ABCB2 | 4 | 5′flanking − 236 | gctttgcgcgcggcgctaac G/T tgtgtagggcagatctgccc | 4 | | |
| ABCB2 | 5 | Intron3 + 408 | aaggaaactgaggccaagac C/T ctaaatgctgaaactgcaca | 5 | | |
| ABCB2 | 6 | Exon4 + 153 | ccctcaccatggtcaccctg A/G tcaccctgcctctgcttttc | 6 | | |
| ABCB2 | 7 | Intron4 + 289 | gtatttctttagcatccaag G/T ggcatagctgtgtctctttc | 7 | | |
| ABCB2 | 8 | Intron4 + 291 | atttctttagcatccaaggg C/G catagctgtgtctctttctc | 8 | | |
| ABCB2 | 9 | Intron5 − 63 | ttccttcaggttaatgactg C/T ggttctttgtgtcccctcca | 9 | | |
| ABCB2 | 10 | Intron7 − 185 | gtctctgcccttgtctttgc C/T gcttcttctatctctactcc | 10 | | |
| ABCB2 | 11 | 3′flanking + 71 | agcgcacttttcagctgcgg G/A tgtctcctcttttatcatcc | 11 | | |
| ABCB2 | 12 | 3′flanking + 129 | aactgcatcaccttttccct T/C aagctttttaattcctatga | 12 | | |
| ABCB2 | 13 | 3′flanking + 459 | cattcagggaggcccaggtc G/A tgtgacgtcgacagttgctg | 13 | | |
| ABCB4 | 1 | exon3 + 3 | aacacccttattttatagat C/T caatgactgagtcaagaatt | 14 | | |
| ABCB4 | 2 | intron3 + 45 | cagcatctctacttatacca T/C gctctgctttaaggttctct | 15 | | |
| ABCB4 | 3 | intron3 + 498 | actcaaataggtggtaggag C/T agagacaattcaatacagac | 16 | | |
| ABCB4 | 4 | intron3 + 515 | gagcagagacaattcaatac A/G gacagaagtcttagatgaga | 17 | | |
| ABCB4 | 5 | intron3 + 1030 | tagttttgcaatgtagaatt G/C aaaaagtgatagatggtgtt | 18 | | |
| ABCB4 | 6 | intron6 + 1437 | gttaagcctgcttcaatcaa G/A ttagttatattcttgttcta | 19 | | |
| ABCB4 | 7 | intron6 + 2449 | ttgacttagcgacactgtta G/A catacttatctttcctgtgt | 20 | | |
| ABCB4 | 8 | intron7 + 451 | ccttgctgcacctgtgctgt A/C taagtttggcttattatagt | 21 | | |
| ABCB4 | 9 | intron7 + 530 | agtagagacaggctggcgat C/G acaccggacagagctaactg | 22 | | |
| ABCB4 | 10 | intron7 − 152 | aacagaatcatgaaattaag T/C tgttaatgatttgaaggcct | 23 | | |
| ABCB4 | 11 | exon8 + 40 | aggataaattgtttatgtcg C/T ctgggtaccatcatggccat | 24 | | |
| ABCB4 | 12 | intron8 + 130 | ctggttgactccagatatca T/C agaaggagttgtaaaattct | 25 | | |
| ABCB4 | 13 | intron8 + 248 | aatacacaggaagcttctaa A/G taaagtaaggaagtcactct | 26 | | |
| ABCB4 | 14 | intron8 + 531 | ctaaagagtgaatggattca A/G tacgtcccttggaactcacc | 27 | | |
| ABCB4 | 15 | intron8 + 4240 | ctgaggttccagcttatctc T/A tagagatgtttacttagtct | 28 | | |
| ABCB4 | 16 | intron8 + 4343 | tgttagaagaaaaaaaggtt C/T atattacaagagggtctgac | 29 | | |
| ABCB4 | 17 | intron8 + 4677 | cccaagatatcttcataact G/C tccatagtgcctagggtgcc | 30 | | |
| ABCB4 | 18 | intron9 + 113 | tttacccagattcacctatt A/G agacttcatctcaaaaaaaa | 31 | | |
| ABCB4 | 19 | intron9 + 982 | tgtcctatacagtttttgtt T/A taagtttagtaaattgatta | 32 | | |
| ABCB4 | 20 | intron11 + 457 | tccagcttgggtgacagagt A/G agacttcatctcaaaaaaaa | 33 | | |
| ABCB4 | 21 | intron11 + 1337 | tactcttggggagcctatca C/G cagggtgggtcagatatagc | 34 | | |
| ABCB4 | 22 | exon12 + 3 | tgtttcttttctgtccagat A/T ctctcggcatttagtgacaa | 35 | | |
| ABCB4 | 23 | intron12 + 1288 | cagaccacactaaccctcag T/C tggacctcaggatgtcagtg | 36 | | |
| ABCB4 | 24 | intron13 + 206 | tgtggataagaaaatagcat G/A tggttagaccatttgtgaaa | 37 | | |
| ABCB4 | 25 | intron13 + 988 | cagtcggtttggaagcttgc T/C accctttcttcacttcctca | 38 | | |
| ABCB4 | 26 | intron13 + (1413-1414) | tttatcttcacttatgtttt (T) ctcagttaagttatgctaat | 39 | | |
| ABCB4 | 26 | intron13 + (1413-1414) | tttatcttcacttatgtttt ctcagttaagttatgctaat | 40 | | |
| ABCB4 | 27 | intron13 + 1931 | cttgcaaatgttgctcttcc A/G caaaaaaaaaaggaaaggat | 41 | | |
| ABCB4 | 28 | intron23 + 784 | agtatctcctaaactcttgc T/C atgcaggaaaaattatttta | 42 | | |
| ABCB4 | 29 | intron25 + 158 | gaaatattttactgtattaa T/C gtctagaacttaaatataag | 43 | | |
| ABCB4 | 30 | intron25 + 2920 | ctgagtcttcctatacatct T/A ttccattcctcggatgctgt | 44 | | |
| ABCB4 | 31 | intron29 + 411 | cttctcttaccttgaattct A/C ggctctcgaactttgacttt | 45 | | |
| ABCB4 | 32 | intron32 + 458 | agaaaatgaaattgccctac T/C gagctaactctgaaagcaca | 46 | | |
| EPHX1 | 1 | intron1 + 110 | tgcaaaatgtgtcttactag C/T ttctagtgcataaaatattg | 47 | | |
| EPHX1 | 2 | intron1 + 143 | aaatattggtggagctcttc G/A ctgtgctgggccagtcacca | 48 | | |
| EPHX1 | 3 | intron1 + 1097 | aatccagagagggagataga T/G tggaagttcaagggtggaca | 49 | | |
| EPHX1 | 4 | intron1 + 1717 | ttccaagacagagcgagggg T/C gctgctggggcgtggtttgc | 50 | | |
| EPHX1 | 5 | intron1 + 1772 | aactcgatgctttctcctcc G/T tctgggtcctaactgcagtg | 51 | | |
| EPHX1 | 6 | intron1 + 2054 | gaaatgtaacaggcaacact A/G tggacacagaaagtagatta | 52 | | |
| EPHX1 | 7 | intron2 + 1414 | atttccaaaatctgtttggg G/T gtaactgaaacacttgggaa | 53 | | |
| EPHX1 | 8 | exon3 + 174 | taccctcacttcaagactaa G/A attgaaggtatgtttgcaaa | 54 | | |
| EPHX1 | 9 | intron3 + 6583 | ctgtcaataccatgaagggg G/C ggcgggggcactaagggtgg | 55 | | |
| EPHX1 | 10 | intron4 + 34 | agaaggttccataactgccc G/A tcctcgccaagggtgggccc | 56 | | |
| EPHX1 | 11 | intron4 + 63 | aagggtgggcccggtgttcc C/T accaggctctccttccggcg | 57 | | |
| EPHX1 | 12 | intron5 + 154 | gcagtgcctgaggcacgttg G/A cttggatcctcctgtctgta | 58 | | |
| EPHX1 | 13 | intron5 + 276 | tgctggaccaagctctggga T/C agccctgagcagaactcccc | 59 | | |
| EPHX1 | 14 | exon6 + 130 | gatgtggagctgctgtaccc C/T gtcaaggagaaggtattcta | 60 | | |
| EPHX1 | 15 | intron8 + 206 | ggtgcctggctcccgggcgg C/A cctcagtaccgctccccagt | 61 | | |
| EPHX1 | 16 | intron8 + 353 | tggccctcccagaaaagaga A/G ggccctcagtgaggggagag | 62 | | |
| EPHX1 | 17 | 3′flanking + 708 | aggtgcagactcatgcactc A/G ggccctcagtgaggggagag | 63 | | |
| EPHX2 | 1 | 5′flanking − (523-522) | aaagtcactggatatgcccc (C) tcccccgccccccaacacgg | 64 | | |
| EPHX2 | 1 | 5′flanking − (523-522) | aaagtcactggatatgcccc tcccccgccccccaacacgg | 65 | | |
| EPHX2 | 2 | 5′flanking − 522 | aaagtcactggatatgcccc T/C cccccgccccccaacacggt | 66 | | |
| EPHX2 | 3 | 5′flanking − 521 | aagtcactggatatgcccct C/T ccccgccccccaacacggtc | 67 | | |
| EPHX2 | 4 | 5′flanking − 516 | actggatatgcccctccccc G/C ccccccaacacggtcttatg | 68 | | |
| EPHX2 | 5 | 5′flanking − 515 | ctggatatgcccctcccccg C/G cccccaacacggtcttatgt | 69 | | |
| EPHX2 | 6 | Intron1 − 74 | tggctgcttctcaatgaata T/C gaacagtgtctgtttccatg | 70 | | |
| EPHX2 | 7 | Intron3 + 72 | gagcattaggtcagaatcca T/C tgaagtgagctttgagatca | 71 | | |
| EPHX2 | 8 | Intron4 + 473 | gtgtgtctctagtttaatct A/G caaaaggtgattgaatggag | 72 | | |
| EPHX2 | 9 | Intron5 + 276 | caagagtgggatgttcaagg C/T catcctgacctcacttttga | 73 | | |
| EPHX2 | 10 | Intron8 + 8 | tctgctcctcccggtgggtg T/C gctgtcttgcagctgtctta | 74 | | |
| EPHX2 | 11 | Intron9 + 1573 | atgtcgtgaagactgatgaa C/T gatggacggctgcactgctc | 75 | | |
| EPHX2 | 12 | Intron10 + 207 | gaacaggatggagatgagct T/C gtttatttgtcttttaatga | 76 | | |
| EPHX2 | 13 | Intron12 + 911 | tgaagagacctcgacatgtc G/T catcccacatactacaggga | 77 | | |
| EPHX2 | 14 | Intron12 + 2425 | atcttctcagctgagcaaac C/T gaggctcagagggcttaacc | 78 | | |
| EPHX2 | 15 | Intron12 + 2460 | ttaaccccaactggcccaag G/A ccaggtacatgattgggtca | 79 | | |
| EPHX2 | 16 | Intron12 − 281 | aagtcctttcaagagattat T/C ataagtagtaccttctcatt | 80 | | |
| EPHX2 | 17 | Intron12 − 268 | agattattataagtagtacc T/G tctcattataggaatattga | 81 | | |
| EPHX2 | 18 | Exon13 + 50 | cctgagtcggactttcaaaa G/T cctcttcagagcaagcgatg | 82 | | |
| EPHX2 | 19 | Intron13 + 1739 | ttgtcgtaacagggttttca G/T atgagcatatttcctttgta | 83 | | |
| EPHX2 | 20 | Exon14 + 33 | atgcataaagtctgtgaagc G/A ggtaagagacatgcttggga | 84 | | |
| EPHX2 | 21 | Intron14 + 314 | ggattgagagcttacctcta T/C gggggtcacctcgtgtatgc | 85 | | |
| EPHX2 | 22 | Intron14 + 878 | attcccttattccttcacac C/T gtctgtcactcattcattca | 86 | | |
| EPHX2 | 23 | Intron14 + 948 | gcacaggctgggtatgaagc T/C ggggctgcatgctcagctac | 87 | | |
| EPHX2 | 24 | Intron15 + 259 | agagggttttcactactttt C/T agtcatggctcctcagagaa | 88 | | |
| EPHX2 | 25 | Intron16 + 459 | tcctcatttgtcaagcagaa G/C atgagtttccaatctctggg | 89 | | |
| EPHX2 | 26 | Intron16 + 645 | gtaagtgaacacactgctac G/A atgaaaatgaccaaactgca | 90 | | |
| EPHX2 | 27 | Intron16 + 985 | gtcattatcatcatatgacc G/A atgaaaatgaccaaactgca | 91 | | |
| EPHX2 | 28 | 3′flanking + 12 | aggtggccttacacacatct T/C gcatggatggcagcattgtt | 92 | | |
| EPHX2 | 29 | 3′flanking + 374 | tgttcacggagaatgcacgg C/T atggggatgaaccctttccc | 93 | | |
| EPHX2 | 30 | 3′flanking + 544 | tagccacctgcctttctccc G/A gcttccctagcagagtttgc | 94 | | |
| GAMT | 1 | intron1 + 429 | ctcggaaagctgagctcagg G/A agacagctgtccccggggtg | 95 | | |
| GAMT | 2 | 3′flanking +626 | cactgacctccttgccctga G/A agaaggccggctcctgtgct | 96 | | |
| NNMT | 1 | 5′flanking − 228 | ataattttcctgacgagctc A/T agtgctcccctctggtctaca | 97 | | |
| NNMT | 2 | intron1 + 44 | ccccactaatgtgagtcata T/C agatggagtctcagggcacg | 98 | | |
| NNMT | 3 | intron1 + 149 | ggataaaaaacgaatattggt A/G tagcgattccacagtttaca | 99 | | |
| NNMT | 4 | intron2 + 158 | agataggcccatgtgtgtgc G/A tgttagtaaatttgtgtatg | 100 | | |
| NNMT | 5 | intron2 + 433 | gctgtagccatccaagccta T/C agaacttggctgtgagtgtg | 101 | | |
| NNMT | 6 | intron2 − 3064 | atcatctgactggtaagttc C/T agttctgtggtaactcaagt | 102 | | |
| NNMT | 7 | intron2 − 260 | atttcatggagggaagtcca T/C ggtagaagcaggctgctagg | 103 | | |
| NNMT | 8 | 3′flanking + 71 | ggctcagtggttggggccca A/G tggttcatctaggacgggac | 104 | | |
| PNMT | 1 | 5′flanking − 390 | aagaggtgaatggctgcggg G/A ggctggagaagagagatggg | 105 | | |
| PEMT | 1 | exon2 − 4 | agctcagcagacctcctggc C/T gtggtgggtagctcctttcc | 106 | | |
| PEMT | 2 | intron4 + 39 | actgtccagacgggagtatc C/T cactgcttggtgagccccac | 107 | | |
| PEMT | 3 | intron4 + 1317 | accgtccccagctggcccca G/A cctcctgacatgggcctctg | 108 | | |
| PEMT | 4 | intron4 + 1355 | ctggagccaggctgcagccg A/C agtgcctggccatcctggcg | 109 | | |
| PEMT | 5 | intron4 + 5925 | gtccaggcactgtggcccta C/T gtgggagtctccagtctcca | 110 | | |
| PEMT | 6 | intron4 + 6028 | ggcagtggtccaaggaccag G/C atggactccctcttctcacc | 111 | | |
| PEMT | 7 | intron4 + 6078 | atctgtaccctcgcggactc C/T acctggcttcgtgccatcac | 112 | | |
| PEMT | 8 | intron4 + 6089 | cgcggactctacctggcttc A/G tgccatcacccccgccagat | 113 | | |
| PEMT | 9 | intron4 + 6379 | tcaggtgtcccctccctcat G/A cctcctcaccctgccctctc | 114 | | |
| PEMT | 10 | intron4 + 7339 | tgtaaggaatcctgccaaga C/T ggcagatgcacacggggtca | 115 | | |
| PEMT | 11 | intron4 + 7619 | ctcctgcacatgtgctccag A/G gaggaaaggcatttgacagg | 116 | | |
| PEMT | 12 | intron4 + 8858 | ggcatgtgtgtgtgtgtgta T/G gtgtgtgagtgtgtgcatgt | 117 | | |
| PEMT | 13 | intron4 + 9029 | tttctggaccagaaagcgtc G/A tcctctgccagggcctcttg | 118 | | |
| PEMT | 14 | intron4 + 9056 | gccagggcctcttgcacttg C/T gggaaagctgagctgagctg | 119 | | |
| PEMT | 15 | intron4 + 9512 | ctgagctgggcagcagcatt A/G ctctgtgtgctgctggcact | 120 | | |
| PEMT | 16 | intron4 + 9523 | agcagcattactctgtgtgc T/C gctggcactggcctggtggg | 121 | | |
| PEMT | 17 | intron4 + 9622 | gacaaagtgtacaacaaggt G/A tctcgaactgggtcagctca | 122 | | |
| PEMT | 18 | intron4 + 10776 | ccattcctgggtcttctttg G/A aggctgaatgaaattccatg | 123 | | |
| PEMT | 19 | intron4 + 10912 | tctgccccactttgctcaga G/C gtgcaacaaggccttcagga | 124 | | |
| PEMT | 20 | intron4 + 11590 | ggacactggcctgatgcaga G/C gtgtggtctctctcctgcag | 125 | | |
| PEMT | 21 | intron4 + 12090 | ggccagggcacccctaccag G/C ctgagtcccacctgtccagc | 126 | | |
| PEMT | 22 | intron4 + 12263 | tacccgccttcccagatgga G/A cgggctgctcatgggactta | 127 | | |
| PEMT | 23 | intron4 + 12448 | tctggtcccctctcctgctt G/A tagtttcctgggctaaaatc | 128 | | |
| PEMT | 24 | intron4 + 12730 | tgggaccagtgccgccacca C/T ggcccaaggacctggtgttc | 129 | | |
| PEMT | 25 | intron4 + 13240 | gggctccaggcacacagcgg T/C cccagtacacctgtcgcttt | 130 | | |
| PEMT | 26 | intron4 + 13494 | tccgtggaactcagagatgg T/C acctccctgcgaggtggggc | 131 | | |
| PEMT | 27 | intron4 + 13817 | aactctcccctgctgctgag A/G cagatcttggagcctcggcc | 132 | | |
| PEMT | 28 | intron4 + 14773 | ccgccctgtgcttcatgccc C/T ctatgcctctcactgcctgg | 133 | | |
| PEMT | 29 | intron4 + 14951 | gtcctgaggcccctcccacc G/A gagcctggggtgccgtcaca | 134 | | |
| PEMT | 30 | intron4 + 16896 | gctgtgactgtcttggagac T/C gggtcttggcgggcctggtg | 135 | | |
| PEMT | 31 | intron4 + 19439 | ccaggagcctctgaggcagc G/A ggggcttctcaaccacacag | 136 | | |
| PEMT | 32 | intron4 + 19559 | attttgtcagcatgtcacgt C/T cctttcataatgaagcaagg | 137 | | |
| PEMT | 33 | intron4 + 20051 | acagcactgcgggagccacg A/G catctgcagacgcatttgat | 138 | | |
| PEMT | 34 | intron4 + 20816 | tggactctctggcgtccatc C/T agccacttcagtgcgacgtg | 139 | | |
| PEMT | 35 | intron4 + 21196 | ggctggctgggccctgggat C/G atcgtgacaggctttagtgg | 140 | | |
| PEMT | 36 | intron4 + 21528 | acaggtgggagccgaggctc G/T ggaggtgggccgggctgagc | 141 | | |
| PEMT | 37 | intron4 + 21596 | ccgcttccccgtgctctggc C/T gtagcagaaagtgtcccact | 142 | | |
| PEMT | 38 | intron4 + 22672 | agcctcccactgccttgtgg C/T tgaggggagggggccgggtc | 143 | | |
| PEMT | 39 | intron4 + 22713 | tctaacgctgtcttctttgt A/T ctgaaaaccaaacaccttct | 144 | | |
| PEMT | 40 | intron4 + 23010 | tgccgggcagcggggaggga G/A ggcgagtggttcccccaagt | 145 | | |
| PEMT | 41 | intron4 + 23588 | gtgcaggcgccctgcatccc C/T gcagccaagttctgggcgga | 146 | | |
| PEMT | 42 | intron4 + 23627 | gacactgccctgagccagga C/T ggtgaggtgggacgccttcc | 147 | | |
| PEMT | 43 | intron4 + 23941 | tgaggggttgggactctaca G/A aggagagtggactcacgggg | 148 | | |
| PEMT | 44 | intron4 + 24091 | gacacctcttcactgtcagc G/T ctgagacacgcccctgccct | 149 | | |
| PEMT | 45 | intron4 + 25348 | caggccagttggaatcctac G/A tagagtgaaagcatctcagc | 150 | | |
| PEMT | 46 | intron4 + 25603 | taagcagttaacactgatgc G/A tgatgaaaattccaacagca | 151 | | |
| PEMT | 47 | intron4 + 31540 | cctccaggtggcaggaacac T/C gtgaggagcatgcaacgtgc | 152 | | |
| PEMT | 48 | intron4 + 31637 | gtgggctgggacgccaggac G/A gtgaggggcttcaaggtgtg | 153 | | |
| PEMT | 49 | intron4 + 31642 | ctgggacgccaggacggtga G/A gggcttcaaggtgtgtttgt | 154 | | |
| PEMT | 50 | intron4 + 35593 | ggaggagctgaaagagctgg G/A gctcgggatcaggtggttca | 155 | | |
| PEMT | 51 | intron4 + 35647 | actttgaggcaccaccgcac C/A tgtccgtgcgtgagggagac | 156 | | |
| PEMT | 52 | intron4 + 35862 | tcccagtggtggctctgtcc C/T cgtctcagccgagcactcag | 157 | | |
| PEMT | 53 | intron4 + 35882 | ccgtctcagccgagcactca T/G cggccagggtggctggactc | 158 | | |
| PEMT | 54 | intron4 + 37141 | ccacaggccggatgccttga T/G acttctcagctgcagggctg | 159 | | |
| PEMT | 55 | intron4 + 38862 | tggagagaccacctcagaca C/G caaggacgggcatgccatgg | 160 | | |
| PEMT | 56 | intron4 + 38872 | acctcagacagcaaggacgg G/T catgccatgggtcccggcag | 161 | | |
| PEMT | 57 | intron4 + 39140 | atgtctcaaatctccctccc C/T gggaaatctaggcacaggtc | 162 | | |
| PEMT | 58 | intron4 + 39635 | caggcccaggagcaggtggg G/T cctcctcacaggagcagggc | 163 | | |
| PEMT | 59 | intron4 + 39713 | actctgagcatgctggctcc C/T tccttctttccagggcagca | 164 | | |
| PEMT | 60 | intron4 + 40436 | cctggttgtgcttcggaccc G/A gaggcagacagaggaggcct | 165 | | |
| PEMT | 61 | intron4 + 47485 | acaatgactgttggagccct C/T gagcaggctgtgtcacgtgg | 166 | | |
| PEMT | 62 | intron4 + 48131 | actgggggatcctgaatccc G/A cctcctgatgccagtggagc | 167 | | |
| PEMT | 63 | intron4 + 48558 | cacagtgtgaactgttaggc C/G acagccacatcttgccggag | 168 | | |
| PEMT | 64 | intron4 + 48702 | gagatgggggcggttcggga G/A gcaaaagcaggaaggcagaa | 169 | | |
| PEMT | 65 | intron4 + 50302 | gcatgtgcatgggcagaggc T/C gttcccatctgagtgggacc | 170 | | |
| PEMT | 66 | intron4 + 54102 | ggccgcgtgctcctgcagcc A/T tgggctcctctggcagttct | 171 | | |
| PEMT | 67 | intron4 + 54220 | cccagggacagatcttctcc G/A ccagacgtctctttctgcct | 172 | | |
| PEMT | 68 | intron4 + 54371 | gcagataatgtgcagctggg G/A tgcatgtggttgttgctccc | 173 | | |
| PEMT | 69 | exon5 + 79 | tggcctgctactctctaagc G/C tcaccatcctgctcctgaac | 174 | | |
| PEMT | 70 | intron5 − 6796 | ggaggaagtcagcttcttac A/C gatggtggctcccagctttc | 175 | | |
| PEMT | 71 | intron5 − 6636 | ttttctcctctcaccttttg T/G gttcagaggcagaggtgtgc | 176 | | |
| PEMT | 72 | intron5 − 6448 | gttgggccaggctctgacag G/A accctcgggaccagctcctg | 177 | | |
| PEMT | 73 | intron5 − 5218 | ggagccctggctgaagaagc C/G ttacgaccaaggcctggagg | 178 | | |
| PEMT | 74 | intron5 − 4824 | ggacaggccgggggttgagc G/A gctgcatgaaggagggaggg | 179 | | |
| PEMT | 75 | intron5 − 4249 | tcaccagagtgatttcctcg C/A ggcaggtgcctggggtagcc | 180 | | |
| PEMT | 76 | intron5 − 4230 | gaggcaggtgcctggggtag C/T cactgggcggggtccatgag | 181 | | |
| PEMT | 77 | intron5 − 4182 | ggagagtaaggggtgggggg G/A cacttaggacagggaagctg | 182 | | |
| PEMT | 78 | intron5 − 3369 | ccaggtggggccgtgtgcct G/C tggcctggtgtgtggcccag | 183 | | |
| PEMT | 79 | intron5 − 2625 | cagggaagctgggccctgaa C/T gagctgggcttttgggccac | 184 | | |
| PEMT | 80 | intron5 − 1200 | attattgtgagcatgggaag A/T gcacatttggtcacacatgt | 185 | | |
| PEMT | 81 | intron6 + 606 | gcctggctagacgcccacca A/G tgaccctgatgatggcagca | 186 | | |
| PEMT | 82 | intron6 + 1229 | tttggtccaggaagggggac G/A gcagccaggagcgtctggat | 187 | | |
| PEMT | 83 | intron7 + 716 | atggagatgtgctcccccgg C/G gggtcagaggacctgcggtc | 188 | | |
| PEMT | 84 | intron7 + 1537 | ctctgggggacgcataagcc G/A cctccagaggacatcagcca | 189 | | |
| PEMT | 85 | intron7 + 1718 | gggcttccaggtgtctgagc T/C ccccggcatgtaggacccca | 190 | | |
| PEMT | 86 | intron7 + 2695 | ggctttgggggaccctggac C/T catttctagaaaacagcctt | 191 | | |
| PEMT | 87 | intron8 + 140 | ccagggctcccaggtcagag C/T ggccatggtagcttacaatg | 192 | | |
| PEMT | 88 | 3′flanking + 178 | tacttaggaggcgtcagggg C/T tcacctggccatggccatgg | 193 | | |
| PEMT | 89 | 3′flanking + 394 | gatgacactgtcattcctaa A/G tgaatggccttgtgctgacc | 194 | | |
| GSTM3 | 1 | 5′flanking − 144 | ccaacgccggcattagtcgc G/T cctgcgcacggccctgtgga | 195 | | |
| ALDH5 | 1 | 5′flanking − 2808 | cgttgcactgtaggactctc C/T ccacgtcccctaatcccatc | 196 | | |
| ALDH5 | 2 | 5′flanking − 2575 | gcagttcccgcggatagaga A/G ggtccggtccttcccgctgt | 197 | | |
| ALDH5 | 3 | 5′flanking − 2537 | tgtgggtgaactgtaaaaaa C/T tgcctgtattcaggaggata | 198 | | |
| ALDH5 | 4 | 5′flanking − 940 | cttcaactaatctgggaaca C/T tacactctgtttaattttca | 199 | | |
| ALDH5 | 5 | 5′flanking − 785 | tgggaaagctgaaaagggat G/T ctgagacctgtggttggggg | 200 | | |
| ALDH5 | 6 | exon1 + 183 | ccgacggtcaaccctaccac T/C ggggaggtcattgggcacgt | 201 | | |
| ALDH5 | 7 | exon1 + 257 | cgtgaaagcagcccgggaag C/T cttccgcctgggtcccccat | 202 | | |
| ALDH5 | 8 | exon1 + 320 | gcggggccggctgctgaacc G/T cctggcagacctagtggagc | 203 | | |
| ALDH5 | 9 | exon1 + 605 | acttgccccggcactcgcca C/T aggcaacactgtggttatga | 204 | | |
| ALDH5 | 10 | 3′flanking + 1527 | aaagtgcaactgtaagaccc G/A tagagaaaaactctggttcc | 205 | | |
| TGM1 | 1 | Exon2 + 179 | tgccgaaatgcggcagatga C/T gactggggacctgaaccctc | 206 | | |
| TGM1 | 2 | Intron9 − 611 | acttaccactctgtcctctc C/T tgccaggcctcttcctgtca | 207 | | |
| TGM1 | 3 | Intron9 − 272 | ccgcacatctgtaccctgcc C/G ccatcctccagcagagcagc | 208 | | |
| TGM1 | 4 | Intron10 + 54 | tcagtcatgggttctctggt C/T ccaacttcaccgctgactga | 209 | | |
| TGM1 | 5 | Intron10 − 51 | aggaggccgggagtcaggcc A/G ccctcagaccctctggctca | 210 | | |
| TGM1 | 6 | Intron12 − 47 | gggagtccctgggggaagcc T/G catgtagggaagcaggcctc | 211 | | |
| TGM1 | 7 | Intron13 + 72 | ggataaggacatcagaggtg G/A gcgctaagccagcagcaggc | 212 | | |
| TGM1 | 8 | Intron14 + 1671 | atctcttacccacaccccca C/G catggtggggaggttcctca | 213 | | |
| TGM1 | 9 | Intron14 + 1691 | ccatggtggggaggttcctc G/A tcctaagggatccgcagagc | 214 | | |
| TGM1 | 10 | Intron14 − 1634 | tccctgcctccctccttcag G/A gagctcagaaacaccttcaa | 215 | | |
| TGM1 | 11 | Intron14 − 1459 | ggaaacccctcagaaccagg T/C tccaagccaaatgctttgcc | 216 | | |
| TGM1 | 12 | Intron14 − 801 | cagaatacaaaagtgggatg G/C gaggcaaggagtcccgttag | 217 | | |
| TGM1 | 13 | Exon15 + 233 | ctcgaggtggagcttagccc T/C gtgccaggagcaatgggact | 218 | | |
| TGM1 | 14 | Exon15 + 369 | ggagtcagtcttcacttgca C/A tgggggaacagatgctaata | 219 | | |
| GGT1 | 1 | intron1 + 85 | ttatccagtaaggtggctcc G/A tcacctcttttcctggtggg | 220 | | |
| GGT1 | 2 | exon3 + 68 | gacggccaggtccggatggt G/T gtgggagctgctgggggcac | 221 | | |
| NQO1 | 1 | 1 intron 1 80 | aggaggttgtaggggcttgg C/A ctgaattttgttccttgact | 222 | | |
| PIG3 | 1 | 5′flanking region − 47 | gggaaggaggaaaggaaaga G/A ggggagggtggttctgctta | 223 | | |
| PIG3 | 2 | intron 2 243 | taacaccggacgcccagcag A/C agtcccagcttcttagaatc | 224 | | |
| PIG3 | 3 | 3′flanking region 282 | agcaggccccagccctgccc G/A ctactcacctgggccccacc | 225 | | |
| NQO2 | 1 | 5′flanking region-434 | tttctgttgcaccacggacc C/G tcattctgtaaccgggatac | 226 | | |
| NQO2 | 2 | 5′flanking region-406 | gtaaccgggataccagccag A/G gatggggagcgggaggcgca | 227 | | |
| NQO2 | 3 | 5′untranslated region-102 | tcctgcggctcctactgggg A/C gtgcgctggtcggaaggtga | 228 | | |
| NQO2 | 4 | intron 1 1919 | tcactcaaatagagctgagt T/C agtcactcagctcttggacc | 229 | | |
| NQO2 | 5 | intron 1 2004 | acaaactcacatgccaccag C/G catatgatgtaaacatgtaa | 230 | | |
| NQO2 | 6 | intron 1 3391 | aaagcagagggctgtgcagg C/T gcccctgcccctaggctagg | 231 | | |
| NQO2 | 7 | intron 1 3456 | caaaggcctcatcctcaggg C/A ggccaactcttctgttttag | 232 | | |
| NQO2 | 8 | intron 1 3595 | actgcccagctttaggttca T/C tcttgtaagtgttgctggtg | 233 | | |
| NQO2 | 9 | intron 1 3596 | ctgcccagctttaggttcat T/C cttgtaagtgttgctggtgt | 234 | | |
| NQO2 | 10 | intron 1 3598 | gcccagctttaggttcattc T/C tgtaagtgttgctggtgtca | 235 | | |
| NQO2 | 11 | intron 1 3651 | ccctgcgctttgaagggatg A/G atgtgacctctcccacattc | 236 | | |
| NQO2 | 12 | intron 1 6036 | tggtgtggcggttcactgat C/T ccccagccttctgctcgatc | 237 | | |
| NQO2 | 13 | intron 2 14 | atggcaggtaatgattcact A/G ttgtggagtaagactttttt | 238 | | |
| NQO2 | 14 | intron 2 192 | gccacgtggaagtgtataaa C/T tatctggaattatcttgttt | 239 | | |
| NQO2 | 15 | intron 2 635 | caccctgtttagcacctagc A/C ccatccctggcctctgccca | 240 | | |
| NQO2 | 16 | intron 2 685 | agtagcaccccctccccacc G/A gctgtgacaaaccaaaatgt | 241 | | |
| NQO2 | 17 | exon 3 139 | ctgatttgtatgccatgaac T/C ttgagccgagggccacagac | 242 | | |
| NQO2 | 18 | intron 3 36 | aatgctctatttataaaaac T/C atctttatgttttttacttt | 243 | | |
| NQO2 | 19 | intron 3 728 | aacgtgggcataaaccacca T/C ctagtgcaaaaaaagcggtg | 244 | | |
| NQO2 | 20 | intron 4 1577 | tgcctctgcacaccccttcc C/T gacaccagccctttctttac | 245 | | |
| NQO2 | 21 | intron 4 1832 | tcggccggccacgtggagcc C/T gctttcctcctcgcacccac | 246 | | |
| NQO2 | 22 | intron 4 2583 | tggtgttacgcacagctcct C/T gtcccctccctgcctgccca | 247 | | |
| NQO2 | 23 | exon 5 330 | ctgtactggttcagcgtgcc A/G gccatcctgaagggctggat | 248 | | |
| NQO2 | 24 | exon 5 405 | atcccaggattctacgattc C/T ggtttgctccaggtatgtgc | 249 | | |
| NQO2 | 25 | intron 5 21 | gtatgtgctcttggataagg A/T tcactatggatagttggagg | 250 | | |
| NQO2 | 26 | intron 5 253 | atggcaaacaagggagtggg T/C caggtgtcaggtgacggggg | 251 | | |
| NQO2 | 27 | intron 6 2435 | ccccccttaaatcatttaac T/C gaatggtatgtaacaggtgt | 252 | | |
| SULT1A1 | 1 | 5′flanking region − 1597 | gcagagtaaagggactcact C/G aagaagaggaacgtgggggt | 253 | | |
| SULT1A1 | 2 | 5′flanking region − 1491 | gaggggtatattcatgaaga G/T tccaggaaaaggtaaagatt | 254 | | |
| SULT1A1 | 3 | 5′flanking region − 1376 | cggtttcatatgttactgat C/T atacaatgagatcctaggtg | 255 | | |
| SULT1A1 | 4 | 5′flanking region − 1375 | ggtttcatatgttactgatc A/G tacaatgagatcctaggtga | 256 | | |
| SULT1A1 | 5 | 5′flanking region − 1370 | catatgttactgatcataca A/G tgagatcctaggtgaaacct | 257 | | |
| SULT1A1 | 6 | exon 1B − 65 | aaccctgcattccccacaca G/A cacccacaatcagccactgc | 258 | | |
| SULT1A1 | 7 | intron 1B 442 | gagccaccctgcctaggcct G/A tgcttttgctgagtcatcag | 259 | | |
| SULT1A1 | 8 | exon 1A − 197 | gctgggggtcccagcaggaa A/G tggtgagacaaagggcgctg | 260 | | |
| SULT1A1 | 9 | exon 1A − 159 | ctggctggcagggagacagc A/C caggaaggtcctagagcttc | 261 | | |
| SULT1A1 | 10 | exon 1A − 95 | gagaccttcacacaccctga C/T gccactgactggctttgtga | 262 | | |
| SULT1A1 | 11 | intron 1A 60 | ctggttttcagccccagccc C/T gccactgactggctttgtga | 263 | | |
| SULT1A1 | 12 | intron 1A 69 | agccccagccccgccactga C/G tggctttgtgagtgcgggca | 264 | | |
| SULT1A1 | 13 | intron 1A 174 | tgtgatggtggtaagggaac G/A ggcctggctctggcccctga | 265 | | |
| SULT1A1 | 14 | intron 6 11 | catgaaggaggtgagaccac C/G tgggaagcttccctccatgt | 266 | | |
| SULT1A1 | 15 | intron 6 17 | ggaggtgagaccacctgtga A/T gcttccctccatgtgacacc | 267 | | |
| SULT1A1 | 16 | intron 6 35 | gaagcttccctccatgtgac A/T cctgggggccggcacctcac | 268 | | |
| SULT1A1 | 17 | intron 6 71 | ctcacagggacccaccaggg T/C cacccagccccctcccttgg | 269 | | |
| SULT1A1 | 18 | intron 8 108 | ttggcagcccccacagcagg C/A ccggattccccatcctgcct | 270 | | |
| SULT1A1 | 19 | intron 6 111 | gcagcccccacagcaggccc G/A gattccccatcctgccttct | 271 | | |
| SULT1A1 | 20 | intron 6 270 | ctccctgccaaagggtgtgc C/T acccagggccacagtcatgg | 272 | | |
| SULT1A1 | 21 | intron 6 488 | ttttacttttcctgaatcag C/T aatccgagcctccactgagg | 273 | | |
| SULT1A1 | 22 | intron 6 509 | aatccgagcctccactgagg A/G gccctctgctgctcagaacc | 274 | | |
| SULT1A1 | 23 | exon 7 600 | ccctctgctgctcagaaccc C/G aaaagggagattcaaaagat | 275 | | |
| SULT1A1 | 24 | exon 7 645 | gagtttgtggggcactccct G/A ccagaggagaccgtggactt | 276 | | |
| SULT1A1 | 25 | exon 8 902 | gctgtgagaggggctcctgg G/A gtcactgcagagggagtgtg | 277 | | |
| SULT1A2 | 1 | 5′flanking region − 547 | tgttctttcttggttctatg G/C atccatgctctgctccaccc | 278 | | |
| SULT1A2 | 2 | 5′flanking region − 425 | tgtgggttgcactgggccag G/A acccctggcaccttcaagac | 279 | | |
| SULT1A2 | 3 | 5′flanking region − 358 | ctttccagggcctgcctatc C/T cagctttctccttcttgcct | 280 | | |
| SULT1A2 | 4 | 5′flanking region − 355 | tccagggcctgcctatccca G/T ctttctccttcttgcctggg | 281 | | |
| SULT1A2 | 5 | 5′untranslated region − 28 | actgcagggcgaggagggcac A/G aggccaggttcccaagagct | 282 | | |
| SULT1A2 | 6 | intron 1A 85 | ctgactggccttgtgagtgc G/A ggcaagtcactcagcctccc | 283 | | |
| SULT1A2 | 7 | exon 2 24 | gagctgatccaggacatctc T/C cgcccgccactggagtacgt | 284 | | |
| SULT1A2 | 8 | intron 2 34 | gccacccaccctctcccagg T/C ggcagtccccaccttggcca | 285 | | |
| SULT1A2 | 9 | intron 5 77 | cagcaaccctgtgtcggcac T/C ccctgcccgcttctccagtg | 286 | | |
| SULT1A2 | 10 | intron 6 684 | actggggtcccaggggtcga G/C gagctggctctatgggtttt | 287 | | |
| SULT1A2 | 11 | 3′untranslated region 895 | gctctgagctgtgagagggg T/C tcctggagtcactgcagagg | 288 | | |
| SULT1A2 | 12 | 3′flanking region 98 | cctccccgctccagctcctc A/T acttgccctgtttggagagg | 289 | | |
| SULT1A2 | 13 | 3′flanking region 817 | ccactgactcggggcttgcc A/C aggctgccagggctggcaaa | 290 | | |
| SULT1A2 | 14 | 3′flanking region 1006 | cctctcccctggaggctgct T/C tacccgctgtgggggcgcat | 291 | | |
| SULT1A2 | 15 | 3′flanking region 1464 | tcccgtagcccaggcaagtt C/T ggtgaccagagagcagcccc | 292 | | |
| SULTX3 | 1 | intron 1 332 | cctgcttctccctttacctg G/T ctggctgtgtgaccttggac | 293 | | |
| SULTX3 | 2 | intron 1 1167 | taggaatggctaagcgtgtc G/A ttggcttctgtggccactca | 294 | | |
| SULTX3 | 3 | intron 1 2872 | cattctcactgatgcagacg G/A aagcttctgggcctgggcgt | 295 | | |
| SULTX3 | 4 | intron 1 6242 | cacccttggcttttaccagc A/G tggaaacattttacctgaat | 296 | | |
| SULTX3 | 5 | intron 1 6601 | gcgtgggcttctggagggag C/T gagaggagagtggagggccc | 297 | | |
| SULTX3 | 6 | intron 1 6768 | agcttgaaatgagccagact C/T tcctgggacctgttgacccc | 298 | | |
| SULTX3 | 7 | intron 1 6905 | agtactttgttttatcctcc C/T catcctcacaactttgccat | 299 | | |
| SULTX3 | 8 | intron 1 7464 | gccaggatcccttgagagac G/A acatgaacacagccaggagc | 300 | | |
| SULTX3 | 9 | intron 1 7833 | tgcttcgggctgggcttggc G/A ggggcagctgtgctccaggc | 301 | | |
| SULTX3 | 10 | intron 1 8189 | caaactggggcccttaatgc C/T gcacaccagagcctcctttc | 302 | | |
| SULTX3 | 11 | intron 1 8316 | ctctcacacaagggcggagc C/G tcttccccttgaggcagagc | 303 | | |
| SULTX3 | 12 | intron 1 8617 | agacagaggctggggccaag C/T cagggttgccggagcttcct | 304 | | |
| SULTX3 | 13 | intron 1 8631 | gccaagccagggttgccgga G/T cttcctggactggtcaggcc | 305 | | |
| SULTX3 | 14 | intron 1 9493 | ttttcctcttagagcttccc G/A tcgtgctctgtgtcgagggc | 306 | | |
| SULTX3 | 15 | intron 1 10306 | caggcggggagcctgaatgc C/T gcagtcgtgagggtggccag | 307 | | |
| SULTX3 | 16 | intron 1 11987 | tcataaaataatgatatcag T/C acactttttggaaatttgag | 308 | | |
| SULTX3 | 17 | intron 1 13085 | ctctgtgcccggtgttgaga C/A aggccatgccctagagtcct | 309 | | |
| SULTX3 | 18 | intron 1 13108 | gccatgccctagagtcctgg G/A gagttccaccccagaacagc | 310 | | |
| SULTX3 | 19 | intron 2 700 | gaaccatctgggagtcgttc C/T gtactgccgtgccgagggcc | 311 | | |
| SULTX3 | 20 | intron 2 818 | agccatagtagctagccagc G/A atcagcgctgggaggggagc | 312 | | |
| SULTX3 | 21 | intron 2 1677 | actccacttcccctgaaccc C/T accccttccttcctcctctg | 313 | | |
| SULTX3 | 22 | intron 4 4954 | gcgtgccgaaggcgggaggg C/T tgggatggctcaagacgtga | 314 | | |
| SULTX3 | 23 | intron 5 3632 | ccagctgactcccacaccag C/T ggtcagagaacattgtcttt | 315 | | |
| SULTX3 | 24 | intron 5 3662 | acattgtcttttaaggtttc C/T gaagtgctgcaataaagaaa | 316 | | |
| SULTX3 | 25 | intron 6 1874 | tctgatctcagagagctgac A/G atggaaagaattctaaacga | 317 | | |
| SULTX3 | 26 | intron 6 2133 | agaccggtgcctgcagttta T/G cccacagctcagccctccct | 318 | | |
| SULTX3 | 27 | intron 6 2524 | ggaagggccagggctgcctg T/C gatgcccagagcagtgcact | 319 | | |
| SULTX3 | 28 | intron 6 2573 | agatcatactcgctcctggg A/G tgtttattaaacacctgcca | 320 | | |
| SULTX3 | 29 | 3′flanking region 12 | gttcccggcgttgcgtcgag C/G gtttctgcttgtgggggtag | 321 | | |
| SULTX3 | 30 | 3′flanking region 445 | tccaaagcctgtcttcctga T/G ttcctgtggaaggagagtcc | 322 | | |
| TPST1 | 1 | 5+ flanking region − 298 | acccgccaccatgcccagct A/C attttttttgtatttttttt | 323 | | |
| TPST1 | 2 | intron 1 3520 | agaaaagcagattaatgtaa C/G agtgacgcttagacaacaag | 324 | | |
| TPST1 | 3 | intron 1 3610 | ggcagaaagagaatatagca A/G ctattaaacacaaataaatt | 325 | | |
| TPST1 | 4 | intron 1 20828 | tattgctgtccacctggtca A/G tgtgtcctgctgataagtgc | 326 | | |
| TPST1 | 5 | intron 1 − 6761 | aatacaatacttattctgta T/C aattctagagggcccagaga | 327 | | |
| TPST1 | 6 | intron 1 − 544 | tagaacaagtgaatatttta G/T gttcttagtggtttatggtt | 328 | | |
| TPST1 | 7 | intron 1 − 526 | tacgttcttagtggtttatg G/T ttggcagttttcccccaaca | 329 | | |
| TPST1 | 8 | intron 1 − 234 | tcaagacatttaataatgca C/T atgtttcagctaaccctttt | 330 | | |
| TPST1 | 9 | intron 1 − 48 | ttatagtgggtttaagcatg A/G tttctaaaaaatttaaataa | 331 | | |
| TPST1 | 10 | intron 2 − 18944 | aaaacattagaactgggaag G/A ttaaaaaatctttagtcttt | 332 | | |
| TPST1 | 11 | intron 2 − 18687 | tatgtgcaccctaataacat A/G tttccttaaaactagtacta | 333 | | |
| TPST1 | 12 | intron 2 − 18501 | ttggaaggtaacttaatgta A/G gtgcctgaaaaacagggata | 334 | | |
| TPST1 | 13 | intron 2 − 159 | gaatggggatttccctcagt C/G ctgcccactggctgctcttg | 335 | | |
| TPST1 | 14 | intron 2 − 19 | acctgttgccttaaactcac G/A cctgctttgtttttccaggt | 336 | | |
| TPST1 | 15 | intron 2 158 | tgctggggaagaaagatcag C/G gtctgggacttgttgatttt | 337 | | |
| TPST1 | 16 | intron 2 3779 | agcagggcacgtcaccctcc G/T ggcacacccatgtgttcacc | 338 | | |
| TPST1 | 17 | intron 2 292 | ttgttattttcattatgaac C/T atgaaatatttcagctgaaa | 339 | | |
| TPST1 | 18 | 3′untranslated region 1518 | gttgtctgtacatgttctaa T/G gttttgtagaacacgtgtgc | 340 | | |
| TPST1 | 19 | 3′flanking region 264 | acggtgcttggcctgcatta C/T cattttgtagtgaagtttct | 341 | | |
| TPST2 | 1 | intron 2 578 | tcacctatcatcctcactgc G/A aggatgccaggatacctccc | 342 | | |
| TPST2 | 2 | intron 2 789 | cttaagccatcgtgcaggtc A/G ttgctgtcttctgctcactt | 343 | | |
| TPST2 | 3 | intron 3 2009 | cccaggctggagtgtagtgg T/C gtgatctcggctcactgcaa | 344 | | |
| TPST2 | 4 | intron 3 2017 | ggagtgtagtggtgtgatct C/T ggctcactgcaacctccgcc | 345 | | |
| TPST2 | 5 | intron 3 2035 | ctcggctcactgcaacctcc G/A cctcccgggttcaagcagtt | 346 | | |
| TPST2 | 6 | intron 4 104 | aatgttcagtctctcaattc C/T tggtcatctgatttgttcct | 347 | | |
| TPST2 | 7 | intron 4 379 | tacatacctaccctattggt C/T cctttcttgtcttataaggt | 348 | | |
| TPST2 | 8 | intron 4 588 | tactgcagcctgatacttct G/T ggcttaagccatcctctcac | 349 | | |
| TPST2 | 9 | intron 4 626 | caccccaggctcctgagtag C/T taggactgcaggtgcacgcc | 350 | | |
| TPST2 | 10 | intron 4 718 | cccaggctggtctagaactc C/G tggccgtacgggatgcccct | 351 | | |
| TPST2 | 11 | intron 4 873 | gttgatggccttatttatac G/A tttccattacagcttctagt | 352 | | |
| TPST2 | 12 | intron 4 949 | caaatatttgaaaatgggac C/G caggcctgaggaagagcttt | 353 | | |
| TPST2 | 13 | intron 4 1033 | taagctcagcatttctgagc G/A tgtgctgattttaggaaata | 354 | | |
| TPST2 | 14 | intron 4 1051 | gcgtgtgctgattttaggaa A/G taaacagttatcgtattgaa | 355 | | |
| TPST2 | 15 | intron 4 1356 | gattcaacgtacataccagc C/T gacattgacaggtgaatggc | 356 | | |
| TPST2 | 16 | intron 4 1707 | gtctccttaaaaggtggctc G/T ctgcccctggcttgccccag | 357 | | |
| TPST2 | 17 | intron 5 215 | aagaccagcctgaccaaaac G/A gtgaaaccccgtctctacta | 358 | | |
| TPST2 | 18 | intron 5 341 | tgggaggcagaggtcgcagt G/A agctgagatcacgccgttgc | 359 | | |
| TPST2 | 19 | intron 6 31 | ggacttcactgggggttccc G/A ctgcttctgggtggccccgg | 360 | | |
| TPST2 | 20 | intron 6 273 | gtttgtctgacactggggac A/G gggcaggaagcaccactatg | 361 | | |
| TPST2 | 21 | intron 6 693 | aaagggatttttttgaactt G/C gtaattcaaagatttaagat | 362 | | |
| TPST2 | 22 | intron 6 1635 | tcctgggtacagagttggcc T/G tgaacaaacatgagtccttc | 363 | | |
| TPST2 | 23 | 3′untranslated region 1147 | cttccccactttcagatctc C/T gcaaatgacttcattgccaa | 364 | | |
| SULT1A3 | 1 | exon 8 843 | cgcttcgatgcggactatgc G/A gagaagatggcaggctgcag | 365 | | |
| CST | 1 | intron 1b 6302 | agagctccccagagaggact A/G tgaggctgcatgatgcatga | 366 | | |
| CST | 2 | intron 2a 1004 | gagtgagacccccatctcta C/T aaaattttttttaaaaagta | 367 | | |
| CST | 3 | intron 2a 1395 | atgcctaagtttacagtagc T/C ggggcccctccatggctgcc | 368 | | |
| CST | 4 | intron 1d 473 | ccagagcctgaggttggtgc T/A gtccctgtctggaccctgct | 369 | | |
| CST | 5 | intron 2b 726 | ctatctctccagtgcctctc T/C gtccctgtctggaccctgct | 370 | | |
| CST | 6 | intron 2b 745 | ctgtccctgtctggaccctg C/A tggggggccacagagcaggc | 371 | | |
| CST | 7 | intron 3 85 | tcactagtttcctgctgctg G/A tgtactcctatgccgtgccc | 372 | | |
| CST | 8 | intron 3 308 | tcgtctgaggtcaggagttc G/A agaccagcctggccaacatg | 373 | | |
| CST | 9 | intron 3 853 | ttttgtcctataaaatggca G/A tttcatgtggcccaagctga | 374 | | |
| CST | 10 | exon 4 198 | gaggcagtgatccgggccaa G/T ggctcggcgggggagtgcca | 375 | | |
| SULT1C1 | 1 | intron 3 2280 | gcaaatttttggtattttta G/T tacagtcagggttttaccat | 376 | | |
| SULT1C1 | 2 | intron 3 3742 | gcagatctcactttctggca G/A attccctgaatttgctcccc | 377 | | |
| SULT1C1 | 3 | intron 3 4453 | ttcatagggcttttccctca C/T ttgttttgtaattttgtata | 378 | | |
| SULT1C1 | 4 | intron 3 5234 | gaaaagagactagaggcagg A/G gagctttgcagttcttctaa | 379 | | |
| SULT1C1 | 5 | intron 3 6175 | tggctggcaggaaggtgagg G/C agtcctctcttctctggtcc | 380 | | |
| SULT1C1 | 6 | intron 3 205 | acatgaaggcaggatccaga T/C tgaatgtttggagggaacta | 381 | | |
| SULT1C1 | 7 | intron 3 408 | ggctcacgcctgtaatccca G/C cactttgggaggccgaggcg | 382 | | |
| SULT1C1 | 8 | intron 3 429 | cactttgggaggccgaggcg G/C gtggatcacaaagtcaggag | 383 | | |
| SULT1C2 | 1 | 5′flanking region − 110 | tcctgttaactcacagagaa C/T ggaagggctggaacgggacc | 384 | | |
| SULT1C2 | 2 | exon 1 15 | acactaatggccttacacga C/G atggaggattttacatttga | 385 | | |
| SULT1C2 | 3 | intron 1 297 | gtagacttgtttatttattc A/C ttcccaatctaggcccttat | 386 | | |
| SULT1C2 | 4 | intron 1 363 | gagtgtgtgagctagaaagg T/G gatcctgagtctgatttggg | 387 | | |
| SULT1C2 | 5 | intron 1 2300 | gggctactatcagcagccac C/T acctcaggaaggatgacttc | 388 | | |
| SULT1C2 | 6 | intron 2 455 | aagacttggaagcaaataga T/G aaaaaaaaaatcgtagaaat | 389 | | |
| SULT1C2 | 7 | intron 4 55 | caaaatctccaaacacccta G/A aaggaaagaatcttttcttt | 390 | | |
| SULT1C2 | 8 | intron 4 111 | ctgccttctttaatggaaca T/C tctcacttctcttcaggaat | 391 | | |
| SULT1C2 | 9 | intron 5 1857 | ctttgtgtttactttgtttt T/C acttggtacaaaagtgttgt | 392 | | |
| SULT1C2 | 10 | intron 5 2082 | ctttgtgtttactttgtttt T/C gtcccacagccacagtgatg | 393 | | |
| SULT1C2 | 11 | intron 5 933 | agctactgaacctctcccac A/G taactgtatttcaggggcag | 394 | | |
| ST1B2 | 1 | intron 1 80 | acttgtccataaaatcatta C/T cattctaaataaagttaata | 395 | | |
| ST1B2 | 2 | intron 2 − 352 | aacatttaaatagtcattta T/C agcaatgcacaggtataata | 396 | | |
| ST1B2 | 3 | intron 2 − 85 | attcataatgctcaaaaaat G/A tcttgaaaaactggttggca | 397 | | |
| ST1B2 | 4 | intron 4 460 | gtacttgacattaaaaaata T/C ctgatgtttatatatccata | 398 | | |
| ST1B2 | 5 | intron 4 470 | ttaaaaaatatctgatgttt A/G tatatccataaatagctaat | 399 | | |
| ST1B2 | 6 | intron 4 518 | tttaagattgtcctcatatt C/G ttacttcctttggttactaa | 400 | | |
| ST1B2 | 7 | intron 4 616 | aatgtttatgaaaatagact T/C ttatctggttttagtggcct | 401 | | |
| ST1B2 | 8 | intron 5 58 | ctgcatcatgctgtaaaagg G/A ttgatatttgctttccaact | 402 | | |
| ST1B2 | 9 | exon 6 612 | taatagaatccaaaggagga A/C atcaagaagatcattagatt | 403 | | |
| ST1B2 | 10 | intron 6 582 | aatacattacttccatttaa G/A tagtctgtttattgtggctt | 404 | | |
| ST1B2 | 11 | intron 6 3130 | agatgtaaaaaattattcaa A/T ttttcaaaagcctgaaaatt | 405 | | |
| ST1B2 | 12 | 3′untranslated region 907 | tttaaagtgtctaaatcaca C/A atctgaagaaatcagagatt | 406 | | |
| ST1B2 | 13 | 3′flanking region 50 | tcagatcccagttttgttcc T/G ttgattctgagtttggaaat | 407 | | |
| ST1B2 | 14 | 3′flanking region 328 | tttgacccaggacactgtgt T/G ccactgctgtctaccgagtt | 408 | | |
| ST1B2 | 15 | 3′flanking region 446 | gtagttcagattttggaaat G/A ttttttctatatcataccta | 409 | | |
| CHST2 | 1 | 5′flanking region − 260 | agccggacagtccgccgggc G/A gtgatccgggggccgctccc | 410 | | |
| CHST2 | 2 | 5′flanking region − 56 | gcgctggggaccagccgccg C/T gcccgcctcggagtcgcggc | 411 | | |
| CHST2 | 3 | 5′flanking region 218 | aggagtgaaacacatctttg T/A attctaaaggcagaaaccaa | 412 | | |
| CHST2 | 4 | 5′flanking region 383 | gcagagaccaatgttttggt G/C ctgaggctggttcagaaaaa | 413 | | |
| CHST2 | 5 | 5′flanking region 952 | tactgaaacattctgcagaa T/C gttatactatgagaagaaat | 414 | | |
| SULT2A1 | 1 | intron 2 478 | ggactgggctctgtacacac T/C tcgtcttactgtgtgtaaat | 415 | | |
| SULT2A1 | 2 | intron 2 382 | caaaaccctcttaatattct G/A tttctatctgtctcagaact | 416 | | |
| SULT2A1 | 3 | intron 2 409 | tctgtctcagaactgattgc A/G tgactctaggatcgctatat | 417 | | |
| SULT2A1 | 4 | intron 2 249 | agctggaaattacaggcaca C/T gccaccacacccagctaatt | 418 | | |
| SULT2A1 | 5 | intron 2 395 | aggcatgagccacggcgccc G/A gccaatttatcagctttaat | 419 | | |
| SULT2A1 | 6 | 3′flanking region 33 | ttccttgttaaaagttacca G/C ggttggccaggcacggtggt | 420 | | |
| SULT2A1 | 7 | 3′flanking region 46 | gttaccagggttggccaggc A/G cggtggttcatgcctgtaat | 421 | | |
| SULT2A1 | 8 | 3′flanking region 199 | ttagccaggcgcattggctc A/G tgtctgtaatcccagcactt | 422 | | |
| SULT2B1 | 1 | intron 2 4162 | ttctcccctctcctcaccat C/T cgcacacaggtgatctacat | 423 | | |
| SULT2B1 | 2 | intron 3 879 | gagggcatccagctctgggg G/A ctggacctgggggtttgtgg | 424 | | |
| SULT2B1 | 3 | intron 4 3882 | ttccacgctccttccttggc C/T catgtccaagcagtaatggc | 425 | | |
| SULT2B1 | 4 | intron 5 1780 | cctgcagaagggggtccctt C/T ttgtgggggcattgagacag | 426 | | |
| SULT2B1 | 5 | intron 5 1814 | taatggctgcagcatggagc G/A ttgtgggggcattgagacag | 427 | | |
| SULT2B1 | 6 | extron 6 789 | ccctcttctccaggggtctg C/T ggcgactggaagaaccactt | 428 | | |
| CHST4 | 1 | 5′flanking region − 1092 | atgaagccttgtgccatctc G/A ctgtgtcgtgccagcacctg | 429 | | |
| CHST4 | 2 | 5′flanking region − 941 | ctgccagagagaaacaggaa G/A ggaggaagagccacacaatt | 430 | | |
| CHST4 | 3 | intron 1 − 150 | caggaaatgatttggagaag G/T actggtgccattgttggcac | 431 | | |
| CHST5 | 1 | intron 1 − 144 | ggcctcttaggtttcagcca A/C gacaggtgactcttagcacc | 432 | | |
| CHST5 | 2 | intron 2 17 | caacgtaagagcgcttctca T/A tgtccagctcctttgtttct | 433 | | |
| CHST5 | 3 | intron 2 139 | aatcccagcactttgggagg C/A ggagatgtgcggatggatca | 434 | | |
| CHST5 | 4 | intron 3 1828 | gactgtatgtctgctattca T/C ataggaacaaataattcatg | 435 | | |
| CHST5 | 5 | intron 3 2037 | aaatgaaaccaacaccaaca C/G tgcagagaagcaaacaaaag | 436 | | |
| CHST5 | 6 | intron 3 2134 | aagcagctaaattgtgttcc G/A tacaggtgcaattaggcagg | 437 | | |
| CHST5 | 7 | intron 3 2528 | atggtaaagttcgcctgggt G/A cagtatgtcagcatcctgct | 438 | | |
| CHST5 | 8 | intron 3 2674 | gcacttatcctagaaaggcc A/G tttctgaagactcagcagga | 439 | | |
| CHST5 | 9 | intron 3 7039 | ctggctcccgccggccaccc T/C gggaccgcagccacgtctga | 440 | | |
| CHST5 | 10 | intron 3 7211 | gtagccccaggacaccccca T/G cctcaacatcccattctggg | 441 | | |
| CHST5 | 11 | intron 3 7294 | ggagcttccagtggcttggt T/C acccccgactcttcgtccat | 442 | | |
| CHST5 | 12 | intron 4 108 | gcagggtcctgcactctgca G/A ggggcaatcacaggtgggag | 443 | | |
| CHST5 | 13 | intron 4 402 | ctggctcccgccggccaccc T/C gggaccgcagccacgtctga | 444 | | |
| CHST5 | 14 | intron 4 547 | gtagccccaggacaccccca T/G gaaggagcagaggtgagatc | 445 | | |
| CHST5 | 15 | intron 4 1142 | gccccaggtctcatagctcc C/G cattggcagtgctgggattt | 446 | | |
| CHST5 | 16 | intron 4 1187 | cactgggcagtaattggggc A/G tgggatgggcatgagggccc | 447 | | |
| HNK-1st | 1 | intron 1 139 | gtgttttggcgacttgaaga C/T ctccctagttcgcgggagta | 448 | | |
| HNK-1st | 2 | intron 1 1020 | acctgagcagaaaattctct T/C cttcgctgaaatgaaaattg | 449 | | |
| HNK-1st | 3 | intron 1 1091 | aagaatttgtaaacatcaca G/A gcaacttgcagttatattcg | 450 | | |
| HNK-1st | 4 | intron 1 1971 | ctataactatttcaaacata C/T gaaacaggcataattggatt | 451 | | |
| HNK-1st | 5 | intron 1 2096 | atttagaatattcatttacc A/C agaaatccaaatataacctg | 452 | | |
| HNK-1st | 6 | 5′untranslated region − 91 | ctatccagtgacaagaggaa C/A caagaacctcagttcagggg | 453 | | |
| HNK-1st | 7 | intron 2 − 530 | agtgggcggaggcgagaagc G/A tcagtgttcattcctttgct | 454 | | |
| HNK-1st | 8 | intron 2 − 466 | gctacatcttgtcagccagt C/T agaattttaaacacagccag | 455 | | |
| HNK-1st | 9 | intron 2 − 92 | acggaaatatttgtgctgat A/T cttactgactgaaatcacct | 456 | | |
| HNK-1st | 10 | intron 3 152 | catggcctccgttccttcat G/A ttacagaggtgtgaggggag | 457 | | |
| HNK-1st | 11 | intron 3 312 | cacagtggccttatgccttg C/T agcagggcgcctctcaggct | 458 | | |
| HNK-1st | 12 | intron 3 1948 | tcctttgatgtatcaagttt T/C gtgctgaatgttttcagtgt | 459 | | |
| HNK-1st | 13 | intron 3 2140 | ttacacctggagaggagcac C/T gcagcggtccttaatactgc | 460 | | |
| HNK-1st | 14 | intron 4 187 | agaagcacattcctgaggaa C/T tgaaggtgggcacagccagg | 461 | | |
| HNK-1st | 15 | intron 4 581 | cctgatcattccctagctgg G/A atgaggggtgcactctggaa | 462 | | |
| HNK-1st | 16 | intron 4 615 | tctggaaggcctctcacttc G/C taacccccattctggatcta | 463 | | |
| HNK-1st | 17 | intron 5 7 | gattgttctaaatggtgtgt G/A tgggtctactgaatgtccac | 464 | | |
| HNK-1st | 18 | intron 5 123 | acctgaagggactggtggcc G/T tccagacaggcctgtttttg | 465 | | |
| HNK-1st | 19 | intron 5 721 | ataattatgggctctgctta T/C gaaatttagcttcagacagg | 466 | | |
| HNK-1st | 20 | intron 5 867 | tgctgcccacagagtcggtg G/A tcactcctggccactgtttg | 467 | | |
| HNK-1st | 21 | intron 6 444 | ccaggagcattttcttccat T/C gaggagatccccgaaaacgt | 468 | | |
| HNK-1st | 22 | intron 6 94 | ctgagttctgtacttggcag A/G ttgatcggaggaccacagag | 469 | | |
| HNK-1st | 23 | intron 6 247 | catgaaggtgacatcatttt G/A ttaatagaaattagcaggca | 470 | | |
| HNK-1st | 24 | intron 7 696 | aggaggaaccggacagagac C/G cgggggatccagtttgaaga | 471 | | |
| HNK-1st | 25 | intron 7 870 | gagaccctggaggacgatgc C/T ccatacatcttaaaagaggc | 472 | | |
| HNK-1st | 26 | 3′untranslated region 1110 | tcaaatatctttattagacc T/C ggggctaaccaggtgaagat | 473 | | |
| HNK-1st | 27 | 3′untranslated region 1178 | ccacacccctcctttgagga C/T gcccggggtctcccacaggc | 474 | | |
| HNK-1st | 28 | 3′untranslated region 1393 | ggaagcatcacacagcgtta G/A gagccgtttccttcaggtgt | 475 | | |
| HNK-1st | 29 | 3′untranslated region 1452 | tgaggttctcctggctagtc A/G gggtggcttcacccatcact | 476 | | |
| HNK-1st | 30 | 3′untranslated region 1540 | gcaagggggctgctgaaatc G/C cagagacttttgcagcatca | 477 | | |
| HNK-1st | 31 | 3′untranslated region 1696 | gggtggtgtggtgtccaggg G/A tccatctttccagaatccat | 478 | | |
| HNK-1st | 32 | 3′untranslated region 1829 | aggggaggctttttctacct G/A agaaggggagtgtctttgag | 479 | | |
| HNK-1st | 33 | 3′untranslated region 2211 | tccagcagtgcggcttcctg G/T caacaaggtaggccctggtg | 480 | | |
| HNK-1st | 34 | 3′untranslated region 2212 | ccagcagtgcggcttcctgg C/T aacaaggtaggccctggtgc | 481 | | |
| HNK-1st | 35 | 3′flanking region 1016 | cacacgaaggtgtgcactca C/T ggcctgcagggcacccaggt | 482 | | |
| HNK-1st | 36 | 3′flanking region 1152 | gcatgctttgctcatctgga A/C tctccagaagcagggaacag | 483 | | |
| HNK-1st | 37 | 3′flanking region 1291 | gccgagaccctcagcaggat A/G gtgcagttacagggctgagc | 484 | | |
| STE | 1 | 5′flanking region − 605 | caggtttctaaaataataat C/T gaaaggtgagtgatgtttac | 485 | | |
| STE | 2 | 5′flanking region − 536 | taaaattttcaggtctgctt A/G agagttaaaggcaaagagtt | 486 | | |
| STE | 3 | 5′flanking region − 231 | ccttcttccccaacccctga C/T gaaaggtgagtgatgtttac | 487 | | |
| STE | 4 | 5′untranslated region − 64 | tgcagcttaagatctgcctt G/A gtatttgaagagatataaac | 488 | | |
| STE | 5 | intron 1 69 | aaatatagaatgaaaattat G/A tattaccaagctcttaaaaa | 489 | | |
| STE | 6 | intron 1 311 | caatgagaaaataaagcaag C/G agggtagaaggaggtagaat | 490 | | |
| STE | 7 | intron 1 655 | tctaagaaagtagggactat G/A agaacccctatgtatctata | 491 | | |
| STE | 8 | intron 1 671 | ctatgagaacccctatgtat C/T tatatccaccatagtattct | 492 | | |
| STE | 9 | intron 1 772 | aaaaggcaggttggaagatg C/A aggaggggagtatgcagaaa | 493 | | |
| STE | 10 | intron 1 1716 | taaccatcttgcttaacctt A/G tcatttttagccaagtcatt | 494 | | |
| STE | 11 | intron 1 1928 | aaatgatacatattcaggaa A/G tcaaaaatctctgacttaga | 495 | | |
| STE | 12 | intron 1 1953 | aaatctctgacttagatacc C/T ggcaataataatcaaatgta | 496 | | |
| STE | 13 | intron 1 2087 | aattttgaaagaaattgaag T/G tctgtggtttttatttatca | 497 | | |
| STE | 14 | intron 1 2323 | taggtatgtaggagggtccc G/C ttatatacatagttgttaat | 498 | | |
| STE | 15 | intron 2 165 | tctattccatgaccacaatt T/G ttacctgtaacttgaatagt | 499 | | |
| STE | 16 | intron 2 1707 | cctaggacccaacatgagac A/G taatataccatcagtaaaat | 500 | | |
| STE | 17 | intron 3 850 | ggtgtccattccctcaagaa T/G ttatactttgtgttacacac | 501 | | |
| STE | 18 | intron 4 1653 | agtaacaggctagtagataa T/C ataaataactgaggccaacg | 502 | | |
| STE | 19 | intron 4 1899 | tacatgaacttagagaatca A/G gtagatcacacacaccaaca | 503 | | |
| STE | 20 | intron 4 1930 | cacaccaacaataaaattac A/G cagaatgataaaagaatttg | 504 | | |
| STE | 21 | intron 5 666 | ttctgatcatgtagtaacaa T/C tataaagaaaataataatgt | 505 | | |
| STE | 22 | intron 5 982 | aggcaaagcagaaccttttg A/C ctcacacaacattatattat | 506 | | |
| STE | 23 | intron 7 369 | agattttattcctctctctt T/C ttgagttgaagaaataagtt | 507 | | |
| STE | 24 | intron 7 447 | cacctttcaagggtaagtgg C/A aaaaaatagaaattcaaata | 508 | | |
| STE | 25 | intron 7 672 | aatcttgctctttgaaccat A/T ctgtcagtgagagtcaggga | 509 | | |
| STE | 26 | intron 7 856 | tgttacagaggacttaaaac A/G gttgtcttgcttgcaaacgg | 510 | | |
| STE | 27 | 3′flanking region 218 | cagcctcccaagtagctagg A/G ctacagacatgtgcaaccat | 511 | | |
| ADH1 | 1 | 5′flanking region − 55 | atcatgtgtggaactggaat C/T gggtgttattcaagcaaaaa | 512 | | |
| ADH1 | 2 | intron 1 268 | acatttgcggtaaagcgata A/G tttattccaagctaatcatg | 513 | | |
| ADH1 | 3 | intron 3 442 | aaatggaggctacatggcta C/A ggctgaatgagcatgacctt | 514 | | |
| ADH1 | 4 | intron 6 56 | tacaacttggaggatgcatt T/G aggctgcagaatatatgttt | 515 | | |
| ADH1 | 5 | intron 8 74 | gtctagcagaaaatgaaaag G/A tggaaggatgagaaaaatta | 516 | | |
| ADH2 | 1 | intron 2 340 | ctattttttaaagcgtgcat T/C cttacataagacttaaatat | 517 | | |
| ADH2 | 2 | intron 3 91 | aaggcaatgagagacgaaag T/G gcttgcacaaggtcaccgcg | 518 | | |
| ADH2 | 3 | intron 3 205 | atgtattgtacccttcaacc A/G ttatgtaccgagtatctact | 519 | | |
| ADH2 | 4 | intron 7 108 | acaattgacaaggcaagatt T/C tgaaaacaaatcaaaaataa | 520 | | |
| ADH3 | 1 | 5′flanking region − 254 | tgagagaagagaagcaggaa C/G ttgagagaggaggaagagag | 521 | | |
| ADH3 | 2 | intron 2 355 | tatgcattcttctatattat A/G caagacaaaaattttaggat | 522 | | |
| ADH3 | 3 | intron 3 32 | acactcagggaacatgcctt G/A gttcaccatcacaagattag | 523 | | |
| ADH3 | 4 | intron 4 6 | ctgcttgaaaaatgagtaag C/T ttctgatgctttctttgcac | 524 | | |
| ADH3 | 5 | exon 5 453 | agcaccttctcccagtacac A/G gtggtggatgagaatgcagt | 525 | | |
| ADH3 | 6 | exon 6 815 | ttcgtttgaagtcatcggtc A/G gcttgacaccatggtatgat | 526 | | |
| ADH6 | 1 | intron 3 249 | tgaaactggacttgaaagta G/A aaatgagacaaaaatttatg | 527 | | |
| ADH6 | 2 | intron 6 815 | ttcgtttgaagtcatcggtc A/G gcttgacaccatggtatgat | 528 | | |
| ADH6 | 3 | exon 7 885 | gtctgtgtggttgttggggt G/A ttgcctgccagtgttcaact | 529 | | |
| ADH6 | 4 | intron 7 1292 | gttgagaaacactgcctagt C/A ccgtctgtggtcctagaatt | 530 | | |
| ADH6 | 5 | intron 7 1616 | ctatcacagaataatccgca T/C agaacactaagcagattacg | 531 | | |
| ADH7 | 1 | 5′flanking region − 528 | tgtgcagacacagaaagttt T/C acttaactttctacccctaa | 532 | | |
| ADH7 | 2 | intron 1 361 | tcagtagcatgtgctgcact C/T gctgcagtagttcaatggga | 533 | | |
| ADH7 | 3 | intron 3 183 | aacctcaacctttagaaggc A/G aaccttacggtgtttataaa | 534 | | |
| ADH7 | 4 | intron 4 76 | tgaattgaattaattaatac G/A tgtatttgatgtatcaaaca | 535 | | |
| ADH7 | 5 | intron 5 615 | tggcatagcgtaaagagact T/A ggaaaaatggaataaagcca | 536 | | |
| ADH7 | 6 | intron 8 532 | aagtctaaccatatcaccaa T/G ttagtatgccattgtactat | 537 | | |
| ADH7 | 7 | intron 8 651 | gctgctatttatttcaagta G/A gccacaaaatttccttattt | 538 | | |
| ADH7 | 8 | intron 8 760 | catttttagatgaagaccaa T/G gttgtgaaagcaaataaata | 539 | | |
| ADH7 | 9 | intron 8 1207 | tctccacatttggtctagcc T/C acaggatcatcatattatga | 540 | | |
| ADH7 | 10 | intron 8 1691 | tccctcatctcattgcccac G/A ctcattgctttaattcagtc | 541 | | |
| ADH7 | 11 | 3′untranslated region 1354 | atttacattttgtaaggcta T/C aattgtatcttttaagaaaa | 542 | | |
| ADH7 | 12 | 3′untranslated region 1498 | gatatagtaaatgcatctcc T/C agagtaatattcacttaaca | 543 | | |
| ADH7 | 13 | 3′untranslated region 1584 | aaacacttgttatgagttaa C/G ttggattacattttgaaatc | 544 | | |
| ADH7 | 14 | 3′untranslated region 1818 | aatataaacatagagctaga A/G tcatattatcatacttatca | 545 | | |
| ADH7 | 15 | 3′flanking region 865 | tacatcaaaagaaataaatc G/T aagaaggaataaacacattt | 546 | | |
| HEP27 | 1 | 5′flanking region − 191 | tcagcactctgtgtctagct A/T aaggtttgtaaatgcaccaa | 547 | | |
| HEP27 | 2 | 5′untranslated region − 163 | gaacccatcaattccgtaca C/A attttggtgactttgaagag | 548 | | |
| HEP27 | 3 | intron 1 1941 | aaatttaccctaaccagcct G/C actctctgccactttctgtt | 549 | | |
| HEP27 | 4 | exon 3 289 | ttgtgtgccacgtggggaag G/A ctgaggaccgggagcagctg | 550 | | |
| HEP27 | 5 | intron 4 1070 | tgtctcagttcacaggatca T/C gactctttttctcgaaactg | 551 | | |
| HEP27 | 6 | 3′flanking region 362 | ggctttgtgtgtgctccatt A/G tctgaactgggcctgctggg | 552 | | |
| L1CAM | 1 | intron 1 + 767 | tttgacttccttacatgggt G/A actgtgtgagtcactctgtt | 553 | | |
| L1CAM | 2 | intron 1 + 862 | gcattgggtcatgtgtatgt G/C tgagtggggctgaatgtaag | 554 | | |
| L1CAM | 3 | intron 1 + 1332 | cagggatgaaggagcagagc C/T gctgagaggccacacaggtg | 555 | | |
| L1CAM | 4 | intron 4 + 502 | tttccctggggttttccctt T/C gcattccatcctccctgagc | 556 | | |
| L1CAM | 5 | intron 18 + 147 | agcgacgttatgaaattccc G/A acacttcacatttctataat | 557 | | |
| L1CAM | 6 | intron 24 + 221 | ctccttagccccccagaggg C/T cccaactttaagagcatact | 558 | | |
| AANAT | 1 | 5′flanking − 542 | aggggtgcaggatggggtgt G/T agctggagggcagggggtag | 559 | | |
| AANAT | 2 | 5′flanking − 263 | ccccccacataagaggtggg C/G ttgtccaagactccgaggga | 560 | | |
| AANAT | 3 | intron3 39 | cgcccagctccagggaggcc T/A ctgaagacagaggtcagcca | 561 | | |
| AANAT | 4 | exon4 150 | cagccggccgtgcgccgggc C/T gcgctcatgtgcgaggacgc | 562 | | |
| ARD1 | 1 | intron1 + 317 | ccgtcggtctgctcggcccc C/G ctccctcggggctgggcagg | 563 | | |
| ARD1 | 2 | intron6 + 322 | gctcctcagcatctgctcac G/A ccagggacccacacctctct | 564 | | |
| ARD1 | 3 | intron6 + 1095 | aaggctccatcctgagacaa A/C aagtccagtgtgacctgccc | 565 | | |
| ARD1 | 4 | intron6 + 1179 | aggaggaagacctgtatccc A/G gggacaccctcctccactcc | 566 | | |
| ARD1 | 5 | intron7 + 159 | cctccaggctgctaggcaga C/T ggcctcctctaaagcccagc | 567 | | |
| ARD1 | 6 | intron7 + 295 | tgaccagccctgccacccga G/T gagccttgggcagaaccctg | 568 | | |
| ARD1 | 7 | intron7 + 416 | actaccatggaggcccccac G/A acagagcgctgccccttgac | 569 | | |
| NAT1 | 1 | 3′UTR 215 | aataataataataataataa A/T aaatgtattttaaagatggc | 570 | | |
| NAT2 | 1 | exon2 867 | cgtgcccaaacctggtgatg G/A atcccttactatttagaata | 571 | | |
| NAT2 | 2 | 3′flank 521 | ccatccatactttgccacaa G/A agaaggaacatgagctttat | 572 | | |
| NAT2 | 3 | 3′flank 573 | gatttgaaatcctgtggaca C/T ggggtgaattacttttaaaa | 573 | | |
| NAT2 | 4 | 3′flank 918 | attttctgtttgtaaattcc A/G gtatcagggctatagtttaa | 574 | | |
| NAT2 | 5 | 3′flank 979 | actattctccctcttcgact C/T gtgatgactataataatctt | 575 | | |
| NAT2 | 6 | 3′flank 1958 | tacctattgaagtaagccta C/T gtcatatccacctatttgtt | 576 | | |
| NAT2 | 7 | 3′flank 2034 | ccactgattcccagagctag T/G tcattaagaagacagtgcct | 577 | | |
| NAT2 | 8 | 3′flank 2201 | cagattactggagggctact G/A tttgctcaccaatgcaaatg | 578 | | |
| NAT2 | 9 | 3′flank 2818 | gggatatttgtctcctttct C/G cccagtgcatgttggaaacc | 579 | | |
| NAT2 | 10 | 3′flank 3237 | atatatattccaattaaaaa A/A caaaataaatttccgaaact | 580 | | |
| NAT2 | 11 | 3′flank 3388 | caacaaagagattttttaaa G/A ctttttaaaacaccagacag | 581 | | |
| NAT2 | 12 | 3′flank 3660 | cagcactattcgcaatagca A/G agatgtggaatcaatctaaa | 582 | | |
| NAT2 | 13 | 3′flank 3973 | agcagaaaaaataaataatg C/T gtactaggcttactacctgc | 583 | | |
| NAT2 | 14 | 3′flank 4029 | caaaacaaccccccatgaca T/C gagtttatctatataacaaa | 584 | | |
| NAT2 | 15 | 3′flank 4118 | ataagattaatatctgcata C/A aaatctttgtttacagcttg | 585 | | |
| NAT2 | 16 | 3′flank 4146 | tgtttacagcttgttatata C/T tgaattatgtctgctccccc | 586 | | |
| NAT2 | 17 | 3′flank 4279 | ttaatctgataggattggtg G/C ctttataagaaaaagaaaag | 587 | | |
| NAT2 | 18 | 3′flank 4323 | ttgctctctccccagtgcag T/G taccaaggaaaggccatgtg | 588 | | |
| NAT2 | 19 | 3′flank 4446 | tcaattggctttatctgcga T/C tctggaatcaggcaatactc | 589 | | |
| NAT2 | 20 | 3′flank 4462 | gcgattctggaatcaggcaa T/C actccatttcataaaacaga | 590 | | |
| GZMA | 1 | 5′flanking − 462 | cctcagcttgcacttggcct A/G ctaattcttatataatccaa | 591 | | |
| GZMA | 2 | 5′flanking − 172 | agcctgcctgctggcagtga G/C ccatcatccaccattctcac | 592 | | |
| GZMA | 3 | intron1 1949 | gacataaggttctctctatc A/T gcatgtatggtttgccttgt | 593 | | |
| GZMA | 4 | intron2 + 683 | gactgcgtgaccaggtggaa C/T tagcctcagcatggaagggt | 594 | | |
| GZMA | 5 | intron2 + 1250 | gttggtgtagtttatactag G/A ttatgaatgatagccttaat | 595 | | |
| GZMA | 6 | exon4 + 105 | tgccaagttgcagggtgggg C/G aggactcacaatagtgcatc | 596 | | |
| GZMA | 7 | intron4 + 696 | atagagccttacctgaagaa A/G ggtgtgcagtatgcatggtt | 597 | | |
| GZMA | 8 | intron4 + 1141 | ctgttcagggaggatcccgg G/A ttccaacatggttctttatt | 598 | | |
| GZMB | 1 | 5′flanking + 529 | gcctccgtctcacaccaaca A/G gcagatttccccaccacggc | 599 | | |
| GZMB | 2 | intron3 + 141 | gagggaagattgtgcagccc C/T atcactgtgtcggggcccag | 600 | | |
| GZMB | 3 | 3′flanking + 448 | ttttcagggcctgtccctcc G/A atgggggcaggcttctccca | 601 | | |
| ESD | 1 | 5′flanking − 323 | gtcttgggacagaggagttg G/A gggagttgaaattaggccct | 602 | | |
| ESD | 2 | intron 1 603 | gtcatttctgatggggtcat C/T agggaaatgggattgagcgc | 603 | | |
| ESD | 3 | intron 1 717 | tgtgtggtagaagcagcatt C/T taagcactacgtgaattaac | 604 | | |
| ESD | 4 | intron 1 1864 | gctttcatgcaggattgatc G/C tagtgggatgtattaggaag | 605 | | |
| ESD | 5 | intron 1 2389 | ttttgggaacacctgtctag G/A tgttaagagccagtggaata | 606 | | |
| ESD | 6 | intron 2 21 | taaacttgttttattgttta G/A tgttaagagccagtggaata | 607 | | |
| ESD | 7 | intron 2 588 | taaaattagtatctctctct G/A taagttcattatttaagata | 608 | | |
| ESD | 8 | intron 2 1498 | tagaaaaatgtgtatcacac C/T gtaagtgttcagtaatgtta | 609 | | |
| ESD | 9 | intron 3 92 | ctttatctagatattatagt C/A cctcattttacttttaaact | 610 | | |
| ESD | 10 | intron 3 422 | gtaaagagattaaacacaca C/T gcacacatacatatacctat | 611 | | |
| ESD | 11 | intron 3 581 | agaaaacctgagaaatgaca C/T aatttatttaaagccatagt | 612 | | |
| ESD | 12 | intron 3 2270 | gccagtaattacatgtagcc G/A tttacatcaaattagctaat | 613 | | |
| ESD | 13 | intron 3 2951 | taatgaaagtaaatgtttca A/G cttccctaacaaaagttgaa | 614 | | |
| ESD | 14 | intron 3 3001 | aaatgtcagaaattttttgt G/A ccgtcagtcatcaacaagaa | 615 | | |
| ESD | 15 | intron 3 3096 | aaggagcatacagaaaactt G/C ccatgatggggcctttgtgg | 616 | | |
| ESD | 16 | intron 4 2611 | tctaatagtccccagtatta A/G tggtgcacatcttcatgtcc | 617 | | |
| ESD | 17 | intron 5 390 | tcttttttcatctctgttaa C/T atcaaccatacagttaaaca | 618 | | |
| ESD | 18 | intron 7 107 | ttagtattggaactaaactt t/C tctagtgttgagaactttgg | 619 | | |
| ESD | 19 | intron 8 1090 | aaattctaactaattaaagg G/T ttcatcctttagtaactaga | 620 | | |
| ESD | 20 | intron 8 1651 | tataaagttgtggttaatga A/G tatatatgaataagaatatt | 621 | | |
| ESD | 21 | intron 8 2047 | agaaggaaaaaggccatttt G/C ttaagaatccctgagatatg | 622 | | |
| ESD | 22 | intron 9 − 3490 | atagaaggagaggctatact A/G cctccttaagtctcaggacc | 623 | | |
| ESD | 23 | intron 9 − 2596 | actaaggataaaaatatggc A/G tactcagtcacattggaact | 624 | | |
| ESD | 24 | intron 9 − 666 | aggccttaatgacatatttc T/C cctcacataaagatacaaca | 625 | | |
| ESD | 25 | intron 9 − 660 | taatgacatatttcccctca A/C ataaagatacaacatgcttt | 626 | | |
| ESD | 26 | intron 10 799 | tatggtaactgaagaaaatg A/G cattaagttcctaaagttat | 627 | | |
| DDOST | 1 | intron2 628 | attctgttaagaagttctta T/C attaagaaatattgtctcct | 628 | | |
| DDOST | 2 | intron2 3125 | gagaatataggagcttctgc G/A tatgcctgaaagtcagtcag | 629 | | |
| DDOST | 3 | intron2 3920 | attactcatttaatgaataa A/G tggattactgagcactgtct | 630 | | |
| DDOST | 4 | intron3 189 | actgctgtccaggggtccat C/T tggggctgagcccagctgga | 631 | | |
| DDOST | 5 | intron6 185 | ctgtcctcttgttcgggagg C/T gtggcagcttttcccttact | 632 | | |
| DDOST | 6 | exon8 37 | aactatgaactagctgtggc C/T ctctcccgctgggtgttcaa | 633 | | |
| DDOST | 7 | intron9 37 | tcctgcccaagaatgctgcc A/A aaaaacggccccaggcctca | 634 | | |
| MGST1 | 1 | 5′flanking − 5 | tctggaccctgaacaggagg G/C gacatcgtgacaaagcaaat | 635 | | |
| MGST1 | 2 | intron1A + 330 | atcagcaggcgatggttact C/G tgggcgggtaaatcaggtga | 636 | | |
| MGST1 | 3 | intron1C + 1428 | gtaaagggaaagggcgttcc T/A caactgagaagtgaagattc | 637 | | |
| MGST1 | 4 | repeat | attatttgctctacctcagg G/A tttttcgggtcaagcgagat | 638 | | |
| MGST1 | 5 | intron1C + 2914 | ctcatcaggtgtgtgtcaga G/T ggcttggtgctggccagtct | 639 | | |
| MGST1 | 6 | intron1C + 4274 | attgtaatagattaacaaag G/T tgatgaaagtagtgtacata | 640 | | |
| MGST1 | 7 | intron1C + 4276 | tgtaatagattaacaaagtt T/G atgaaagtagtgtacataat | 641 | | |
| MGST1 | 8 | intron1C + 4306 | gtgtacataatgtacatagt A/G tagttgaacacatagcaagc | 642 | | |
| MGST1 | 9 | intron1C + 4406 | gatggctatatgaccaataa T/A gatacatataaatgtataga | 643 | | |
| MGST1 | 10 | intron1C + 4464 | agaaagattgcagctgatag A/G tgtcaggctaataaggacac | 644 | | |
| MGST1 | 11 | intron1C + 4683 | aatggcagaggactggaaat G/T tacattttaagctttaccct | 645 | | |
| MGST1 | 12 | intron1C + 4767 | gccttcctcttcagcacatt C/T ccaattatacttccaattcc | 646 | | |
| MGST1 | 13 | repeat | atttcaatttttttttttgg G/A gggggagacagagtctcact | 647 | | |
| MGST1 | 14 | repeat | aattacctcccaaaggcctc A/T tatcccagatactatcacat | 648 | | |
| MGST1 | 15 | intron2 + 2379 | ttctcaaatttcattataca C/G tattcttcaacccaaagttt | 649 | | |
| MGST1 | 16 | intron2 + 2767 | tttaactatagatgccttct T/G ctcctcttgtgtttgattta | 650 | | |
| MGST1 | 17 | repeat | tcactgcagcctcaacctct C/T gggctcaggtgatcctccaa | 651 | | |
| MGST1 | 18 | repeat | aaaaaaatttgtagatatgg T/G tactccctatgttgcccagg | 652 | | |
| MGST1 | 19 | repeat | ctccctatgttgcccaggct A/G atcttgaattcttgggctca | 653 | | |
| MGST1 | 20 | intron3 + 1495 | gtcagacaatggccttcagc G/A tcctctctttgcagaatatg | 654 | | |
| MGST1 | 21 | intron3 + 2528 | ttttggagacacttttcaga G/C agagcgtttccagcatcttc | 655 | | |
| MGST1 | 22 | intron3 + 2567 | tccctttccatttttaagtt A/Δ gacttttttttttcacctct | 656 | | |
| MGST1 | 23 | intron3 + 2731 | atacacatatggaacaatta A/C ctaaaaacttaaggtaatat | 657 | | |
| MGST1 | 24 | intron3 + 3288 | gggtttatagtgttcccccc C/Δ tcccccgccccaaaagaccc | 658 | | |
| MGST1 | 25 | intron3 + 4288 | ccattctatttgtcaactgc G/A taacacaggcgtagaagtgg | 659 | | |
| MGST1 | 26 | intron3 + 4378 | aaatgtctgtccttttggca T/C gttgtgaaggagaacactaa | 660 | | |
| MGST1 | 27 | intron3 + 4429 | attggaggtgacgatatctc T/C gtgatgctgggggagaaatc | 661 | | |
| MGST1 | 28 | intron3 + 4817 | attgctatagaagagagtaa C/T gtaaagcagaaatagttttc | 662 | | |
| MGST1 | 29 | intron3 + 6077 | tttgaaattagtgtctttaa T/C agttatctttttccacagag | 663 | | |
| MGST1 | 30 | exon4 + 304(3′UTR) | aagaattctgtacttccaat T/G tataatgaatactttcttag | 664 | | |
| MGST1 | 31 | 3′flanking + 1581 | tctgtgtgcatgaacatgca C/T gcgtgcacgcgcacacacac | 665 | | |
| MGST1 | 32 | 3′flanking + 1729 | tatgtggagcaatttgaaaa A/T agtatattctaagccattaa | 666 | | |
| MGST1 | 33 | 3′flanking + 3407 | ggatcactgctaaagatccc G/A gagtcactccatgtcccagt | 667 | | |
| MGST1 | 34 | intron1B + 36 | ggagaaggggaccgcatgca G/A agggtggcaggcagggaggg | 668 | | |
| MGST1 | 35 | 3′flanking + 25 | gggtaaacccattttgaata T/C tagcattgccaatatcctgt | 669 | | |
| MGST1 | 36 | exon4 + 266(3′UTR) | aaagaaaatcatacaactca G/A catccagttggctttttaag | 670 | | |
| SULT1A2 | 1 | intron 4 1728 | tcagcttcctcctttgccaa A/Δ ccaagagatgagctggcctg | 671 | | |
| SULT1X3 | 1 | intron 1 6415 | tgacctctccctgttagtgt G/Δ ggggcagctctttccagtgt | 672 | | |
| SULT1X3 | 2 | intron 5 2457 | gcccttaaagggaagttcat C/Δ cttctctgccttccaggctc | 673 | | |
| PIG3 | 1 | 5′untranslated region − 93 | tccgcgaggatacagcggcc (CCTGY) cagacaatatgttagccgtg | 674 | | |
| ADH2 | 4 | intron 7 + 108 | acaattgacaaggcaagatt T/C tgaaaacaaatcaaaaataa | 675 | | |
| ADH2 | 5 | intron 3 + (1721-1723) | actgcatagaaatttaagaa GAA/Δ cttgttttattcctctccag | 676 | | |
| ADH2 | 6 | 3′untranslated + (2305-2306) | gttaatgctttcccactctc AG/Δ gggaaggatttgcattttga | 677 | | |
| ADH5 | 1 | 5′flanking − 115 | taactgctgtaaagttacac G/A gggaagccctttcccgacaa | 678 | | |
| ADH5 | 2 | 5′flanking − 114 | aactgctgtaaagttacacg G/A ggaagccctttcccgacaaa | 679 | | |
| ADH7 | 16 | intron 8 + 727 | ttcagatccctgtaagccag G/A tattatttttaccattttta | 680 | | |
| GSTM1 | 1 | 5′flanking − 694 | tacgaagtggctaatttaca C/T agtacttagccagatgaccg | 681 | | |
| GSTM1 | 2 | 5′flanking − 661 | gatgaccgaaggactcagta C/T ccgagggcccctaacagaaa | 682 | | |
| GSTM1 | 3 | 5′flanking − 658 | gaccgaaggactcagtaccc G/A agggcccctaacagaaaaca | 683 | | |
| GSTM1 | 4 | 5′flanking − 656 | ccgaaggactcagtacccga G/A ggcccctaacagaaaacaca | 684 | | |
| GSTM1 | 5 | 5′flanking − 537 | tagaggggagactaagccct G/C ggagtagctttcggatcaga | 685 | | |
| GSTM1 | 6 | 5′flanking − 525 | taagccctgggagtagcttt C/G ggatcagaggaagtcctgct | 686 | | |
| GSTM1 | 7 | 5′flanking − 465 | aattaaattcccaggttggg G/A ccaccactttttagtctgac | 687 | | |
| GSTM1 | 8 | 5′flanking − 383 | gcggagagaaggctgaggga C/T accgcgggcagggaggagaa | 688 | | |
| GSTM1 | 9 | 5′flanking − 382 | cggagagaaggctgagggac A/T ccgcgggcagggaggagaag | 689 | | |
| GSTM1 | 10 | 5′flanking − 378 | gagaaggctgagggacaccg C/T gggcagggaggagaagggag | 690 | | |
| GSTM1 | 11 | 5′flanking − 343 | agggagaagagctttgctcc G/A ttaggatctggctggtgtct | 691 | | |
| GSTM1 | 12 | intron 2 + 118 | tgctggagctgcaggctgtc T/C cttccctgagccccggtgag | 692 | | |
| GSTM1 | 12 | intron 2 + 233 | agtgagtgcccggtctcctc T/C ctgctcttgcttatgggaag | 693 | | |
| GSTM1 | 12 | intron 2 + 26 | tgtgggtggctgcaatgtgt G/A gggggaaggtggcctcctcc | 694 | | |
| GSTM1 | 12 | intron 2 + 140 | actatcagcagttattctca C/T gactccaatgtcatgtcaac | 695 | | |
| GSTM1 | 12 | intron 2 + 577 | ctgccaccccattagaagga A/G ctttctactttccctgagct | 696 | | |
| GSTM1 | 12 | intron 2 + 645 | gctggtctggatccagaggc T/A gccaggtgcttgggcgctcc | 697 | | |
| GSTM1 | 12 | exon 2 + 519 | caccgtatatttgagcccaa G/C tgcttggacgccttcccaaa | 698 | | |
| GSTM1 | 12 | exon 2 + 528 | tttgagcccaagtgcttgga C/T gccttcccaaatctgaagga | 699 | | |
| GSTM1 | 12 | intron 2 + 2421 | cagcaccgtgtagaatcttc A/G taagtgttagctgttactgt | 700 | | |
| GSTM1 | 12 | 3′flanking + 42 | atttgctcctggccatctac C/T cagactgtctgtctgtctgt | 701 | | |
| GSTM2 | 1 | intron 1 + 7 | ggaacatccgcggggtgagc C/G agggtccgctgggcggtggg | 702 | | |
| GSTM2 | 1 | intron 1 + 45 | gggacgggggtgcgtggggg C/T ggggaagtgtggagcagctg | 703 | | |
| GSTM2 | 1 | intron 3 + 70 | gactgcatctcctctcccca G/C cttagaggtgttaagatcag | 704 | | |
| GSTM2 | 1 | intron 3 + 224 | agcaggccctggtctcctct T/C tgcccttgcatatgggaagg | 705 | | |
| GSTM2 | 1 | intron 5 + 100 | ttgattccttctggtgagtt C/A ttggtcttgctgactctaag | 706 | | |
| GSTM2 | 1 | intron 5 + 341 | tcctcttggtgggttcatgg T/C ctggctggcttcaggagtga | 707 | | |
| GSTM2 | 1 | intron 5 + 696 | acctttagctagacacagag C/T gctgatttgtgcatttacaa | 708 | | |
| GSTM2 | 1 | intron 5 + 723 | ttgtgcatttacaatccttt A/G gctaggcagaaaagttctcc | 709 | | |
| GSTM2 | 1 | 3′untranslated + 1006 | tcctcttggtgggttcatgg T/C ctggctggcttcaggagtga | 710 | | |
| GSTM2 | 1 | 3′flanking + 139 | ttctgctgggcatagtaagg C/T gcttgagaattcttgctccc | 711 | | |
| GSTM3 | 2 | 5′flanking − 144 | ccaacgccggcattagtcgc G/T cctgcgcacggccctgtgga | 712 | | |
| GSTM3 | 3 | intron 7 + 165 | agcctaacttctataccttg A/G aggcactgtctacaaaaaaa | 713 | | |
| GSTM3 | 4 | intron 7 + 257 | ctgttggactgggtggggtc T/G ttataagattggtgtatttt | 714 | | |
| GSTM3 | 5 | exon 8 + 91 | cccagtggggcaacaagcct A/G tatgctgagcaggaggcaga | 715 | | |
| GSTM4 | 1 | intron 4 + 67 | ttggctggattggggtgcta T/C gctcagcgtgagtctgtgtt | 716 | | |
| GSTM4 | 2 | intron 7 + 77 | gatgctttcccagtcctgga T/G ctgcataaagaataacttgc | 717 | | |
| GSTM4 | 3 | intron 7 + 80 | gctttcccagtcctggatct G/A cataaagaataacttgcatt | 718 | | |
| GSTZ1 | 1 | 5′flanking − 546 | agcagggcccaccagccgac C/A gcctcgaagcgccgtgagcc | 719 | | |
| GSTZ1 | 2 | 5′flanking − 321 | tgtctgaccagccgccccgc T/C aaggagtcacaagagggcag | 720 | | |
| GSTM2 | 3 | intron 1 + 2890 | aaaatactgcatcaaaacca G/A gccacgctctgttgggggga | 721 | | |
| GSTM2 | 4 | intron 1 + 2896 | ctgcatcaaaaccaggccac G/A ctcgttgggggggacaccaa | 722 | | |
| GSTM2 | 5 | intron 2 + 255 | tctcccaacactgctctcca A/G agccccttggcaaccatgtt | 723 | | |
| GSTM2 | 6 | intron 2 + 1560 | caccactgtttaaggccctg G/C gggggcagagttaaacacaa | 724 | | |
| GSTM2 | 7 | intron 3 + 94 | ccttgaaaggcatcgactac G/A agacggtgcccatcaatctc | 725 | | |
| GSTM2 | 8 | intron 4 + 297 | agaaggaggagtttgctggc C/T ctgtcccctctggtccaggg | 726 | | |
| GSTM2 | 9 | intron 6 + 94 | tatctgaaccagcctcccag G/A ctgctttgggcctgacagtt | 727 | | |
| GSTPi | 1 | intron 1 + 269 | ctcccccgggctccagcaaa C/G ttttctttgttcgctgcagt | 728 | | |
| GSTPi | 2 | intron 1 + 134 | ccccgggcctccttcctgtt C/T cccgcctctcccgccatgcc | 729 | | |
| GSTPi | 3 | intron 1 + 438 | gtgtgtgcgcgtgcgtgtgc G/A tgtgtgtgcgtgtgtgtgtg | 730 | | |
| GSTPi | 4 | intron 1 + 162 | cccgctggctgagtccctag C/T ccccctgccctgcagatctc | 731 | | |
| GSTTi | 1 | 5′flanking − 103 | taaagagtgtcccaggcgtc C/T gtgccgcccaatggggcaca | 732 | | |
| MGST1L1 | 1 | 5′flanking − 105 | tgctgccgctgccgtggggc G/A ttctggagggtggagtgtgg | 733 | | |
| MGST1L1 | 2 | intron 1 + 277 | agtgtctgtgagagaagcag G/A ttctggagggtggagtgtgg | 734 | | |
| MGST1L1 | 3 | intron 1 + 8030 | ggggttatacagagcccctc C/G gcccccaccacacatatgca | 735 | | |
| MGST1L1 | 4 | intron 1 + 8499 | gtatggcaggagtggggtcc C/T ggcaagccatagaggtatgg | 736 | | |
| MGST1L1 | 5 | 3′untranslated + 468 | cgccacctgtgaccagcagc T/G gatgcctccttggccaccag | 737 | | |
| MGST2 | 1 | 5′flanking − 46 | ggtcagcattcaaagtcaag A/T agcgccatttatcttcccgt | 738 | | |
| MGST2 | 2 | intron 1 + 176 | ggtcacccatgccgcctgct A/C ccctccttcccaggggcaag | 739 | | |
| MGST2 | 3 | intron 1 + 204 | tcccaggggcaagcagagac T/C gagaacattccagagattag | 740 | | |
| MGST2 | 4 | intron 1 + 373 | ttacaagtgttccaaaggaa A/T cgtgcctgcttctaaacctg | 741 | | |
| MGST2 | 5 | intron 2 − 3245 | cctcgtgctttgcccacctc G/A gcctcccaaagtgctgggat | 742 | | |
| MGST2 | 6 | intron 2 − 1998 | aggccgaggtgggcggatca T/C gaggtcaggagatcgagacc | 743 | | |
| MGST2 | 7 | intron 2 − 1640 | tgtttattccttgcatagcc A/G taatataaagtatgaatttt | 744 | | |
| MGST2 | 8 | intron 3 + 41 | actgtgttctaatgatgact A/G tgatgcttaaacgattaagg | 745 | | |
| MGST2 | 9 | intron 3 + 453 | atcagagtgctatgttgcag A/G tatatgaactttggcttcat | 746 | | |
| MGST3 | 1 | 5′flanking − 520 | acaaaaaggccctaacagcg A/C taaatccattcacttcggga | 747 | | |
| MGST3 | 2 | 5′flanking − 355 | cgcctaaaaccgctacggtg G/A ctctgctggggacaaattat | 748 | | |
| MGST3 | 3 | 5′flanking − 234 | ctgggggagtagatatatgt T/A tttgagaatgagaggagtaa | 749 | | |
| MGST3 | 4 | intron 1 + 74 | agcctttgcgcaggcactcc C/T atatttcagcctatgcgagc | 750 | | |
| MGST3 | 5 | intron 1 + 682 | agaaaatgccccttctttat G/C tggggtggcagcacggagcc | 751 | | |
| MGST3 | 6 | intron 1 + 832 | cgagtttacaagctacataa T/C agcgtcgggggcaagtaagt | 752 | | |
| MGST3 | 7 | intron 1 + 1919 | aataaaattcctgagtttct G/C tcactcgctcttacagtacc | 753 | | |
| MGST3 | 8 | intron 1 + 1991 | tgtaattaggcaacaggaaa A/G ttgtactatctttcaaatgc | 754 | | |
| MGST3 | 9 | intron 1 + 4458 | tcttccatcctcctaacata T/C agttagcttccactctccaa | 755 | | |
| MGST3 | 10 | intron 1 + 4676 | tgaatatgcaatgcaattgt C/G gggggatagttacttttcat | 756 | | |
| MGST3 | 11 | intron 3 + 278 | cagcatgacccatctaaacc G/C atgttgactctcccaggcct | 757 | | |
| MGST3 | 12 | intron 4 + 423 | cttgcctttttgttgtgggg T/G gtggggtggtcacagagaag | 758 | | |
| MGST3 | 13 | intron 4 + 506 | gtgcagagaagaaaacaaag T/C ggggaaggtggaaaggggat | 759 | | |
| MGST3 | 14 | intron 4 − 162 | tcacagatattttattttcc C/T gactgaaactaacttaattc | 760 | | |
| MGST3 | 15 | intron 4 − 130 | acttaattctacctaatttg C/G gtggggagtagttggccaaa | 761 | | |
| MGST3 | 16 | intron 4 − 105 | ggagtagttggccaaatcat C/G aaattgttaactttttgcta | 762 | | |
| MGST3 | 17 | intron 4 − 65 | aacatattgtgtaatcaacc C/T taggtgttaaaaaaggtttg | 763 | | |
| MGST3 | 18 | intron 5 + 105 | atcccagcactttgggaggc G/C aaggcaggcagattgcttga | 764 | | |
| MGST3 | 19 | intron 5 + 197 | aaaaaatacaaaaattagcc G/A gatgtggtggtgcacacctg | 765 | | |
| MGST3 | 20 | intron 5 + 222 | tggtggtgcacacctgtagt C/T ccagctacttgggaggctga | 766 | | |
| MGST3 | 21 | intron 5 + 374 | tcttatgctactatattttt T/C ttcttgggaatttgagaaaa | 767 | | |
| MGST3 | 22 | 3′untranslated + 517 | atgacttacctttatttcca G/T ttacattttttttctaaata | 768 | | |
| MGST3 | 23 | 3′flanking + 166 | agtctgattgtggtgatgta G/T gtatagtcatgccacagtga | 769 | | |
| GSTA1 | 1 | 5′flanking − 266 | ttgcaaaaagagcaaaatct C/A ggtgaaatgtattgtgtaaa | 770 | | |
| GSTA1 | 2 | intron 2 + 1220 | gagacacaggctttcctaag A/C tatgacaacaccataactag | 771 | | |
| GSTA1 | 3 | intron 4 + 1813 | aaaggcacccactggaggtg A/C attattttgccatcacctga | 772 | | |
| GSTA1 | 4 | intron 5 + 732 | gaagagtgttgtcatgaagg T/C ggagtcactgcccaagggag | 773 | | |
| GSTA1 | 5 | intron 6 + 333 | ttatcccatatgtgcccaca A/G tgagccggtctgagcagagc | 774 | | |
| GSTA1 | 6 | 3′flanking + 412 | ctttcttatgcatttgcaaa A/C caatgattctgtctgctgtg | 775 | | |
| GSTA4 | 1 | intron 1 + 280 | gcattggtggaaggtgggct C/T ggatcgtccccgggcctgga | 776 | | |
| GSTA4 | 2 | intron 3 + 176 | ggaaatcacttcttattcaa T/C agttccataaaagctggccg | 777 | | |
| GSTA4 | 3 | intron 4 + 94 | acaccacatttactttatgt C/G ttacatagttagtgagatca | 778 | | |
| GSTA4 | 4 | intron 5 + 1062 | cacacttgtgcacatgcaga C/T acccatgggcatccaagagt | 779 | | |
| GSTA4 | 5 | intron 6 + 487 | cagatgtgattttactccaa A/G ccattttagctctagaagag | 780 | | |
| GSTA4 | 6 | intron 6 + 595 | tgagctctgagagcaaatga G/A agatgttagcaccctaaaca | 781 | | |
| GSTA4 | 7 | intron 6 + 630 | taaacatcaccccaaaggat T/A cctaccattctccttctgag | 782 | | |
| GSTA4 | 8 | intron 6 + 3943 | tcttcgtagtatctaatacc T/C tttttgttagccttaaagtt | 783 | | |
| GSTA4 | 9 | 3′untranslated + 1099 | taataacaaccgaatgtcta G/A taaatgactctcctctgagc | 784 | | |
| GSTA4 | 10 | intron 5 + (370-371) | gttgtcgaacagctgtctca (TA) gctgacatcctccctgataa | 785 | | |
| GSTA4 | 10 | intron 5 + (370-371) | gttgtcgaacagctgtctca gctgacatcctccctgataa | 786 | \ | |
| NDUFA1 | 1 | 5′flanking − 1437 | agggctaaaaatcctgatta T/A acctaccttgaagcttttaa | 787 | | |
| NDUFA1 | 2 | intron 2 + 3071 | aataaaagtacatggcatat C/A tttgatgggaacagacttgt | 788 | | |
| NDUFA1 | 3 | 3′flanking + 1218 | aactccatgtgtataaagca A/G caccacagatgacacttcca | 789 | | |
| NDUFA1 | 4 | 3′flanking + 1411 | ggattgtgccatcccttgat C/T/G ggcaatgaccttttactttt | 790 | | |
| NDUFA1 | 5 | 3′flanking + 1411 | ggattgtgccatcccttgat C/T/G ggcaatgaccttttactttt | 791 | | |
| NDUFA2 | 1 | intron 2 + 1087 | aacatacaaaaattagccgg A/G tatggtggcgggcacctgta | 792 | | |
| NDUFA2 | 2 | intron 2 + 1089 | catacaaaaattagccggat A/G tggtggcgggcacctgtaat | 793 | | |
| NDUFA2 | 3 | intron 2 + 1356 | ttccctgaaacaacccattg T/C ggccatccagaatcagccaa | 794 | | |
| NDUFA2 | 4 | 3′flanking + 467 | cacagcctcatgggtcagcc C/T actccagagggtgcattccc | 795 | | |
| NDUFA2 | 5 | 3′flanking + 744 | ggaagcaggggccctggcca C/T agccgctggcagtaagcagg | 796 | | |
| NDUFA2 | 6 | 3′flanking + (844-845) | tatagtctacaaagaatgaa (ACAC) aaagatcataacaatagcta | 797 | | |
| NDUFA2 | 6 | 3′flanking + (844-845) | tatagtctacaaagaatgaa aaagatcataacaatagcta | 798 | | |
| NDUFA3 | 1 | intron 2 + 2656 | tccctgctgccctcccctgc G/A cactttatcttccctttgcc | 799 | | |
| NDUFA3 | 2 | exon 4 + 241 | agggccccagcctggagtgg G/G tgaagaaactgtgagcacct | 800 | | |
| NDUFA3 | 3 | 3′flanking + 1019 | tccttacctgcactggcacc A/G gctctggagccccagtccct | 801 | | |
| NDUFA5 | 1 | intron 3 + 2155 | agactctagcatggtacctg G/G aacataaggttccttagaaa | 802 | | |
| NDUFA5 | 2 | intron 3 + 2493 | ggcatattgctagttttctc G/T gtctcaatttcatcatctat | 803 | | |
| NDUFA5 | 3 | intron 3 + 2712 | acaaattttgaactgttcac C/T taacacaggctttttctgaa | 804 | | |
| NDUFA5 | 4 | 3′flanking + 1298 | aggtatctaaaaggtattgc A/C atttggtcattggttctttc | 805 | | |
| NDUFA5 | 5 | intron 3 + (30-31) | aagtcagttttgttgtcttg (GATTTGTGGTATCCAG) tgtaa | 806 | | |
| | | | catttaaccaaaaaa | | | |
| NDUFA5 | 5 | intron 3 + (30-31) | aagtcagttttgttgtcttg tgtaa | 807 | | |
| | | | catttaaccaaaaaa | | | |
| NDUFA5 | 6 | intron 3 + (427-428) | attaagtagcagttaataaa AG/Δ tctagactgctgattcatac | 808 | | |
| NDUFA5 | 7 | intron 3 + (4733-4734) | tataggaattttaaaatata TA/Δ ggatattgaaacattcagtt | 809 | | |
| NDUFA6 | 1 | 5′flanking − 1148 | tttataatttatatatgtta C/T gtgctttcttttgtatagct | 810 | | |
| NDUFA6 | 2 | 5′flanking − 363 | actaccaaggagcgcggcgg G/A cagccggatagcaggacgct | 811 | | |
| NDUFA6 | 3 | exon 1 + 26 | ggggagcggcgtccgccaag C/T tacttctaccgccagcacct | 812 | | |
| NDUFA6 | 4 | intron 1 + 1318 | attcagcagtttgaaaacat A/G atgtttgcctggcagaatac | 813 | | |
| NDUFA6 | 5 | intron 2 + 562 | agttaaagaatctgaaaagt G/C tcagaaatgatttaccctga | 814 | | |
| NDUFA6 | 6 | 5′flanking − (861-862) | ctgtaaaatggggatgctga (T) ggtacctacctgacctatga | 815 | | |
| NDUFA6 | 6 | 5′flanking − (861-862) | ctgtaaaatggggatgctga ggtacctacctgacctatga | 816 | | |
| NDUFA7 | 1 | 5′flanking − 731 | accaaccaaaggtctatcaa A/G ggggtgtcctctttgcaccc | 817 | | |
| NDUFA7 | 2 | 5′flanking − 434 | aaagggaaccatcagaaccc C/T gtgatgaaatgagaatcggc | 818 | | |
| NDUFA7 | 3 | 5′flanking − 395 | gctcccggattccggctggc A/G ggggttagggcagggtagag | 819 | | |
| NDUFA7 | 4 | 5′flanking − 100 | agaggagtcacgtgcttcgg G/A gagagcctttataggacgtt | 820 | | |
| NDUFA7 | 5 | intron 1 + 92 | tcacctccctcctaagccgg G/A acccttcgctctccccgaat | 821 | | |
| NDUFA7 | 6 | intron 1 + 133 | ctccctgggaacccccagct A/C gtcaccccttcagcccggga | 822 | | |
| NDUFA7 | 7 | intron 1 + 136 | cctgggaacccccagctagt C/G accccttcagcccgggaccc | 823 | | |
| NDUFA7 | 8 | intron 2 + 89 | tcctttagacccctgaaacg G/C agggctgacatcctgccacc | 824 | | |
| NDUFA7 | 9 | exon 3 + 196 | gccgccgggaatctgtgccc C/G cttccatcatcatgtcgtcg | 825 | | |
| NDUFA7 | 10 | intron 3 + 4203 | gcctccacccctggggcgcc T/G cctccatcaccccaccctcc | 826 | | |
| NDUFA7 | 11 | intron 3 + 4604 | gggccttgtgtacgctggag A/G ccaaaagtgggaagggagga | 827 | | |
| NDUFA7 | 12 | 5′flanking − (1360-1353) | agggtccagggtcccctgct (CAGAGGCT) aacactggccg | 828 | | |
| | | | aagagaaag | | | |
| NDUFA7 | 12 | 5′flanking − (1360-1353) | agggtccagggtcccctgct aacactggccgaagagaaag | 829 | | |
| NDUFA7 | 13 | 5′flanking − (1240-1239) | tgatagagccctgatccacc CA/Δ ctctctgaaacttctttgct | 830 | | |
| NDUFA7 | 14 | intron 2 + (4142-4143) | cattttgtgactgaggtgac AG/Δ gggcccacagcggggccatg | 831 | | |
| NDUFA8 | 1 | intron 1 − 75 | tttgtgttctctattctgac C/T ctttatccagaagtgagcag | 833 | | |
| NDUFA8 | 2 | intron 2 + 790 | caaacctagacaaagtgtgc C/T attgctcaggcctgagatgg | 834 | | |
| NDUFA8 | 3 | intron 2 + 900 | ttcaggagataaaaagctct G/A attgctcaggcctgagatgg | 835 | | |
| NDUFA8 | 4 | intron 2 + 3837 | gaagttgtcttgtaagtgag A/G taagaatatgtactcacata | 836 | | |
| NDUFA8 | 5 | intron 2 + 3942 | tcattgttttgcaaagagat G/T cccctaacccagctttcttt | 837 | | |
| NDUFA8 | 6 | intron 3 − 66 | gaggagacaccaggaggcgc A/G ttgatggttacagattcctc | 838 | | |
| NDUFA8 | 7 | 3′untranslated + 520 | tttatttctggaccaagtaa A/G gatgggtccgtggcccacac | 839 | | |
| NDUFA8 | 8 | 3′flanking + 367 | gtcatacaaggggagcctcc A/G ggatagaagtgcagaaactt | 840 | | |
| NDUFA8 | 9 | 3′flanking + 777 | attcttttttcactactagg C/T tgtttcctccacatctgact | 841 | | |
| NDUFA8 | 10 | 3′flanking + 1053 | aaagaaaaagcactgtgtga T/A ctgccatggccgcttctgca | 842 | | |
| NDUFA8 | 11 | 3′flanking + 1190 | gattctctaatgaaaaataa G/T acttttttttgcattttttt | 843 | | |
| NDUFA8 | 12 | intron 2 + (449-453) | ggtcattgtgcatgatacttaa (GTAAA) aaaaaactaagctgtgtaat | 843 | | |
| NDUFA8 | 12 | intron 2 + (449-453) | ggtcattgtgcatgatacttaa aaaaaactaagctgtgtaat | 844 | | |
| NDUFA8 | 13 | intron 2 + (707-708) | ctcattttggaaagactctc (A) accttgctgtaccaaaaatg | 845 | | |
| NDUFA8 | 13 | intron 2 + (707-708) | ctcattttggaaagactctc accttgctgtaccaaaaatg | 846 | | |
| NDUFAB1 | 1 | intron 1 + 8451 | cagcaccctgtagaggcctc G/A ggatgctgaagatgccatga | 847 | | |
| NDUFAB1 | 2 | intron 1 + 8495 | gacacaggcattctgcagac G/A ctagacaattttagtggcag | 848 | | |
| NDUFA9 | 1 | 5′flanking − 807 | gatggctctttgtagaacaa T/G gcagattctcaaaggtgacc | 849 | | |
| NDUFA9 | 2 | 5′flanking − 769 | accacagttaaagaaaaaat T/C acaagccattgcgctagaga | 850 | | |
| NDUFA9 | 3 | 5′flanking − 353 | cacaccctattttggtttct C/G ttctccacttttcccctcgt | 851 | | |
| NDUFA9 | 4 | 5′flanking − 322 | ttcccctcgttcttgtcccc C/T cttttctctctcctgggccc | 852 | | |
| NDUFA9 | 5 | intron 1 + 447 | attcatatgagcacaatgga A/G atgataatattacaatacca | 853 | | |
| NDUFA9 | 6 | intron 1 + 1039 | ggcttgatgttcagcctgag G/A caagaattaggagtgtttag | 854 | | |
| NDUFA9 | 7 | intron 1 + 4010 | aatgtatccaaaagagattc T/G cattcctgccatatgaagaa | 855 | | |
| NDUFA9 | 8 | intron 3 + 49 | gacaaatataaattactaag G/A tcatttttaggagtgatagg | 856 | | |
| NDUFA9 | 9 | intron 3 + 107 | aatttcttcccagaatggac C/T aaaggcatcctctgttccca | 857 | | |
| NDUFA9 | 10 | intron 3 + 1183 | atctctggtaatattcatac A/G gattatttgtaatcccttta | 858 | | |
| NDUFA9 | 11 | intron 3 + 1395 | attcctagttctttgtccct C/T aagtttgttggtcaccttgt | 859 | | |
| NDUFA9 | 12 | intron 3 + 2363 | agaaaatagtcatgaatggc C/T ccaactaacactagtcttta | 860 | | |
| NDUFA9 | 13 | intron 3 + 2608 | gtcatttgattacctgagta A/C agtgtactgttacctgtttg | 861 | | |
| NDUFA9 | 14 | intron 4 + 561 | attttataaattctttgatg A/C cttgggggtcttattcaact | 862 | | |
| NDUFA9 | 15 | intron 4 + 860 | attgtgtagagtaatgacag C/T agagctgtcaacttttttaa | 863 | | |
| NDUFA9 | 16 | intron 4 + 879 | gcagagctgtcaactttttt A/T aaaaaataattttagcttaa | 864 | | |
| NDUFA9 | 17 | intron 4 + 893 | ttttttaaaaaaataatttt A/G gcttaaaaaaattaaaaatt | 865 | | |
| NDUFA9 | 18 | intron 4 + 1090 | atcattgctgtttaaaagtt T/C aagtagtgtgaatttcagta | 866 | | |
| NDUFA9 | 19 | intron 4 + 1188 | aaccaatccttttatttttt A/T tcttccagaaactttgattt | 867 | | |
| NDUFA9 | 20 | intron 5 + 161 | gggtgtgtgtgatgttttga C/T gttttgattgattgccttct | 868 | | |
| NDUFA9 | 21 | intron 5 + 373 | ctttctcaccccttgcactg C/T agtggttttgtgccactctt | 869 | | |
| NDUFA9 | 22 | intron 5 + 457 | gccagggaagatgcctattc A/C cacagtgcttatgctccttt | 870 | | |
| NDUFA9 | 23 | intron 5 + 3113 | gatttttctccttcttcaat G/A taagcttcccttaaaataaa | 871 | | |
| NDUFA9 | 24 | intron 5 + 3339 | tctaaactcaaaacaggttt G/A tttggttattgtttaggctg | 872 | | |
| NDUFA9 | 25 | intron 6 + 414 | tatagttttgccttttccag G/C atattacatatatggttaga | 873 | | |
| NDUFA9 | 26 | intron 6 + 518 | ctttcatttcttttcatagc T/C tggtagctcatttctttata | 874 | | |
| NDUFA9 | 27 | intron 6 + 974 | ggattatgcgtacttggaaa A/G tacttggatagcggtgatta | 875 | | |
| NDUFA9 | 28 | intron 7 + 368 | acattaattttgatggagta T/G cacaatgcctccagaggctg | 876 | | |
| NDUFA9 | 29 | intron 8 + 954 | gcatgcaatcagttatatag T/C ctagataagaattacaattc | 877 | | |
| NDUFA9 | 30 | intron 8 + 1253 | tcctcttgaaattgtagata G/T gtatctacacatttctcatc | 878 | | |
| NDUFA9 | 31 | intron 8 + 11608 | gaaaagatagatgtataaat G/A accaaaaattcgtgaagaaa | 879 | | |
| NDUFA9 | 32 | intron 8 + 11930 | ctacaaatatattctaaatg C/T gtaatcatggataagtacaa | 880 | | |
| NDUFA9 | 33 | intron 9 + 1998 | tgtttttcaagcctttaaac G/A gctgtggaaccctgtgctca | 881 | | |
| NDUFA9 | 34 | intron 9 + 2238 | ccagctacttgggaggctga A/G gtgggaggatcacttgagcc | 882 | | |
| NDUFA9 | 35 | intron 9 + 2885 | acagcggtctgtcttcctgc A/G gttctcataggctagattac | 883 | | |
| NDUFA9 | 36 | intron 10 + 801 | tacactaaagtgtctcttac G/A tttatacttgagaaagtgtt | 884 | | |
| NDUFA9 | 37 | intron 10 + 910 | tgcagactttcaggtgggta G/C gatgagggattgctgctgct | 885 | | |
| NDUFA9 | 38 | intron 10 + 1180 | aaaactgagtcagaacgccc G/A tgctcagaaaaacagggcgt | 886 | | |
| NDUFA9 | 39 | intron 10 + 554 | gtgccagcacttaggaatta T/G gaccttctaatgaagttctt | 887 | | |
| NDUFA9 | 40 | 5′flanking − (1129-1128) | taaacagtaggggcaagata (TC) gagtggaaacagccaagatt | 888 | | |
| NDUFA9 | 40 | 5′flanking − (1129-1128) | taaacagtaggggcaagata gagtggaaacagccaagatt | 889 | | |
| NDUFA9 | 41 | 5′flanking − 341 | tggtttctcttctccacttt T/Δ cccctcgttcttgtcccccc | 890 | | |
| NDUFS1 | 1 | 5′flanking − 3 | tcctagggggtcgtcgtggt C/G cagacagtttagcagaacag | 891 | | |
| NDUFS1 | 2 | intron 1 − 445 | gtgttagcaatggctcacgc T/C tctgtttgttgtccttgttt | 892 | | |
| NDUFS1 | 3 | intron 1 + 470 | tttgttgtccttgtttgttt G/T gtccattgaccacgttggac | 893 | | |
| NDUFS1 | 4 | intron 1 + 502 | acgttggacagcattttttt A/G ttcctttaactaacgggaaa | 894 | | |
| NDUFS1 | 5 | intron 1 + 557 | ttttgaaaagttagcccagg A/G ttgcattgcaaataacaaaa | 895 | | |
| NDUFS1 | 6 | intron 1 + 5218 | tatctcagaatatctcagga A/G catttagtagacagctatgc | 896 | | |
| NDUFS1 | 7 | intron 3 + 1371 | aagccctaaaatagatagtg T/G caatgggaatgaaaacaaga | 897 | | |
| NDUFS1 | 8 | intron 5 + 414 | ttttgaaacgaggtctcact A/G tgttgtccaggctgggcttg | 898 | | |
| NDUFS1 | 9 | intron 10 + 812 | gagtgcggtggcgcgatctc G/A atctcgggtcactgcagcct | 899 | | |
| NDUFS1 | 10 | intron 11 + 233 | ggaggccaaggcaggcagat C/T gcctaagtgcaggagtttga | 900 | | |
| NDUFS1 | 11 | intron 11 + 283 | ggccaaacatggcgaacccc G/A tctctactaaaaatacaaaa | 901 | | |
| NDUFS1 | 12 | intron 11 + 585 | ctgtatgtcttaattttaaa G/T taaatttgcattttatatat | 902 | | |
| NDUFS1 | 13 | intron 12 + 1251 | gcaccactgtttaatgctag A/G attcgaaagaggttggtaat | 903 | | |
| NDUFS1 | 14 | intron 13 + 5159 | attacttttagaaaacgtgt T/C ttagctgatactcaggcata | 904 | | |
| NDUFS1 | 15 | intron 14 + 250 | aaaaattgttatattagtta C/T acccttggttcaaaaatgca | 905 | | |
| NDUFS1 | 16 | intron 14 + 550 | gataaagtctcactatgttg C/T ccaggttgatctcaaactcc | 906 | | |
| NDUFS1 | 17 | intron 14 + 2429 | ctgaaaatacaaaaattagc C/T gggtgtggtggcatgtgcct | 907 | | |
| NDUFS1 | 18 | intron 14 + 2530 | ttacagtgagccgagatcac G/T ccactgcgctccagcctggg | 908 | | |
| NDUFS1 | 19 | intron 14 + 2659 | acacatttaattttttacat T/C gaaaatactgcagttatggt | 909 | | |
| NDUFS1 | 20 | intron 16 + 150 | agaaaacatgtattcagaaa C/T aggaattcaaggttacagtg | 910 | | |
| NDUFS1 | 21 | intron 18 + 279 | cactgtgtagcaatttatgg T/C gaattttccaaagtggcaaa | 911 | | |
| NDUFS1 | 22 | 3′flanking + 182 | tctaggataattataattaa T/A aataatcatagtaacaatgg | 912 | | |
| NDUFS1 | 23 | intron 11 + 3226 | aaatgtattgtctgtgcttt T/Δ aacattttgtaatagtaaat | 913 | | |
| NDUFS3 | 1 | 5′flanking − 194 | tctgccacaaggagctagga C/T cacgctcacctcacgatttc | 914 | | |
| NDUFS3 | 2 | intron 1 + 46 | cggggtcaggcgcagcggcg T/C gcccagtgcagagagctcct | 915 | | |
| NDUFS3 | 3 | intron 6 − 439 | aaagctgtgtcaaatgtact G/A ctttagatctggactgtgaa | 916 | | |
| NDUFS3 | 4 | intron 6 − 280 | ggtgggtgagcagtcagttc G/A gagctcctgatgtgggagtg | 917 | | |
| NDUFS4 | 1 | 5′flanking − 439 | aactgaatacagccctgtcc T/A gagggcttgcaaagtgaatc | 918 | | |
| NDUFS4 | 2 | intron 1 + 1829 | gaaaaaaaatcttaatgcca G/T ggaagacgttttttaaatac | 919 | | |
| NDUFS4 | 3 | intron 1 + 2057 | attaatgggaaaatctacat C/G taaaattcattttattgtaa | 920 | | |
| NDUFS4 | 4 | intron 1 − 521 | ttcattttaactaattttat T/G tctcccattttgtgaatggg | 921 | | |
| NDUFS4 | 5 | intron 1 − 1259 | ataaaattatgatattatta G/A tactaatatagccagccata | 922 | | |
| NDUFS4 | 6 | intron 1 − 1174 | aatatatataattataggaa T/C ctcagagtagcaaccatggt | 923 | | |
| NDUFS4 | 7 | intron 1 + 10682 | cacaatataggcacaaactt A/C ctaccaaagcactaacaagt | 924 | | |
| NDUFS4 | 8 | intron 1 + 12299 | tttactatatagatatatgg A/T atagactatagagtatctct | 925 | | |
| NDUFS4 | 9 | intron 1 + 12550 | accaaataaggtattatgca G/A gctcatctttttatataaga | 926 | | |
| NDUFS4 | 10 | intron 1 + 18801 | ggaaagacttgctttgccag T/C gtatccgaaacctctgttat | 927 | | |
| NDUFS4 | 11 | intron 1 + 19888 | tcgcacagctgagaagagca A/G ggggctggttttcagtaccc | 928 | | |
| NDUFS4 | 12 | intron 1 + 20178 | agaaaagatgagtataattc G/A tctaacttacccattcttaa | 929 | | |
| NDUFS4 | 13 | intron 1 + 23016 | ctactctgtgaaagtaaggt T/A atgttgaacaagtaaattaa | 930 | | |
| NDUFS4 | 14 | intron 1 + 23124 | actttctttggagatggagt T/A ccagcagttgggaatgtaat | 931 | | |
| NDUFS4 | 15 | intron 1 + 766 | tgtgatgatttttttttttt T/Δ ggctgtattaaccttccatt | 932 | | |
| NDUFS4 | 16 | intron 1 + 1261 | tttctttctctttttttttt T/Δ gagatacattctcactctga | 933 | | |
| NDUFS5 | 1 | intron 1 + 388 | ccaaacatagccagcacttc C/T ggctgtaactccgggctgtt | 934 | | |
| NDUFS5 | 2 | intron 1 − 13082 | agtgagccgagattgcacca G/A tgcattccagcctgggcaac | 935 | | |
| NDUFS5 | 3 | intron 1 − 12905 | gttttcaacaaaggactcca G/T agtagtagagaagtttctgt | 936 | | |
| NDUFS5 | 4 | intron 1 − 12564 | attttcatcacacctcaact T/G aaggtataacagccttaaga | 937 | | |
| NDUFS5 | 5 | intron 1 − 12561 | ttcatcacacctcaacttaa G/A gtataacagccttaagaatg | 938 | | |
| NDUFS5 | 6 | intron 1 − 10561 | aacaatgtggtatagtgggg C/G gggtggtgagcaggtgtcat | 939 | | |
| NDUFS5 | 7 | intron 1 − 9065 | cctgatgctcctggctccag G/A gtagaccttttccctttaga | 940 | | |
| NDUFS5 | 8 | intron 1 − 8871 | tcaccacgtgtctgtagata T/C aggaccgcagaccttcgctt | 941 | | |
| NDUFS5 | 9 | intron 1 − 7312 | aaatccttggcttctagaat G/T ggtcactgatggtatataat | 942 | | |
| NDUFS5 | 10 | intron 1 − 6827 | aacctctgcctccccgattc A/G cgccattctcctgcctcagc | 943 | | |
| NDUFS5 | 11 | intron 1 − 6725 | agtagagacggggtttcacc G/A tgttagccagcatggtctcg | 944 | | |
| NDUFS5 | 12 | intron 1 − 6631 | aggcgtgagccactgcgccc G/A gcctagaccttcttcttata | 945 | | |
| NDUFS5 | 13 | intron 1 − 6531 | cccaacagctcccaatgtaa A/G acagatctattaatattctg | 946 | | |
| NDUFS5 | 14 | intron 1 − 6346 | gcaacagatcttgacctata T/C cccatagggtacagctgagg | 947 | | |
| NDUFS5 | 15 | intron 1 − 6327 | atcccatagggtacagctga G/C gactttaatcagaaaaggag | 948 | | |
| NDUFS5 | 16 | intron 1 − 6122 | tagccttgcttttactctac T/C gttcctcccaaatcacaccc | 949 | | |
| NDUFS5 | 17 | intron 1 − 2512 | acaaactcttaatgcgaatt T/C tgcagatcaaagtgggctta | 950 | | |
| NDUFS5 | 18 | intron 1 − 1945 | tttaatctcctttaaatttc G/A caatttcacaacctagggta | 951 | | |
| NDUFS5 | 19 | intron 2 + 75 | tttttttttttttttgagac G/A aagtctcactcttgtcccct | 952 | | |
| NDUFS5 | 20 | intron 2 + 148 | ctgtagcctctgcctcccag G/A ttcaggcgattcgcgtacct | 953 | | |
| NDUFS5 | 21 | 3′flanking + 150 | cagattcaagtggttctcct G/G cctcagcctcccaagtagct | 954 | | |
| NDUFS5 | 22 | intron 1 − (10682-10681) | attataaacactaaacaaac AT/Δ gtgtggtctctttagagggg | 955 | | |
| NDUFS5 | 23 | intron 1 − 10272 | aggaacaagtgactaccctg A/Δ aaaaagaagagatgaaacaa | 956 | | |
| NDUFS5 | 24 | intron 1 − 2069 | accagacagagttcccttta C/Δ ttgttttcctgtggcaaaga | 957 | | |
| NDUFS6 | 1 | intron 1 + 26 | ggccgctgggtacaggatgc A/C ccttcctccagccgcacctc | 958 | | |
| NDUFS6 | 2 | intron 2 + 1078 | ggatcatggtggtggagagg G/A gcttgtgtctggtgggtttg | 959 | | |
| NDUFS6 | 3 | intron 2 + 1260 | cagttgtcgagtaagtggtg T/C atagggtaagtgctctttct | 960 | | |
| NDUFS6 | 4 | intron 2 + 1413 | caaaggagctcatggcattg C/T gaatgggacatttcttccgt | 961 | | |
| NDUFS6 | 5 | intron 2 + 1568 | tggagaaggggaggtttctc T/C tagtgtggatgcggtatggt | 962 | | |
| NDUFS6 | 6 | intron 2 + 1692 | gaccgtggtgacggaggttt C/T ctgggcatcgatgggtggtt | 963 | | |
| NDUFS6 | 7 | intron 2 + 6488 | tagcttaaataattattggc A/G ttcatgttcagaatgcctga | 964 | | |
| NDUFS6 | 8 | intron 2 + 6563 | tttaaacttttattttaaat G/A tccatgaatggggtcggtat | 965 | | |
| NDUFS6 | 9 | intron 2 + 6740 | aaagatttaaacctacatat C/T tttatgcccaatcatttgat | 966 | | |
| NDUFS6 | 10 | intron 2 + 6832 | gcgaggactcatttttacag A/T ggttggacacttcactgtgt | 967 | | |
| NDUFS6 | 11 | intron 2 + 7054 | ttcactgccggagcttggcc G/A tgtgaacccggagccgggct | 968 | | |
| NDUFS6 | 12 | intron 2 + 7186 | ggtcagggtcacccttgagc T/C gcgcacactaaatgacggga | 969 | | |
| NDUFS6 | 13 | intron 2 + 7225 | gagggcatcccgcgtcagtc G/A ccagtgtcgaggcgtcagca | 970 | | |
| NDUFS6 | 14 | intron 2 + 7810 | cttccactctggggcgggga C/T gctgtagaaggagcacaaag | 971 | | |
| NDUFS6 | 15 | intron 2 + 11080 | gtaactgttcagtgctttct C/T ctttggatttcatgtaaatc | 972 | | |
| NDUFS6 | 16 | intron 2 + 11657 | gggacagaacgatgtggtgg G/A gagaagagggcgtggcagag | 973 | | |
| NDUFS6 | 17 | intron 3 + 208 | cgaaaaccccctttcaactg T/C gaagtggtgggcggcatgtt | 974 | | |
| NDUFS6 | 18 | intron 3 + 1031 | ctagagtgggactgggcacc C/T ggcatgtcccctcctgggct | 975 | | |
| NDUFS6 | 19 | 3′flanking + 270 | gcttcagagagccaaggtgg G/C tcttgaggtgcatagtgaag | 976 | | |
| NDUFS8 | 1 | 5′untranslated − 45 | agtgtagcctccgcctcccg A/C ttgactggcctgcttggcaa | 977 | | |
| NDUFS8 | 2 | intron 1 + 163 | aggtgcagcggggagccggc T/C ctcagggcgcatgcgccgcc | 978 | | |
| NDUFS8 | 3 | intron 3 + 123 | tctctgagcctgtttccact T/C ttaaaatgattatggtgatg | 979 | | |
| NDUFS8 | 4 | intron 6 − 505 | aggcaaggcaggccgggcac G/A gtggctcacgcttgtaatcc | 980 | | |
| NDUFS8 | 5 | 3′flanking + 491 | ggccctgagctggcctgcgt C/A cagccacatcctctttcctg | 981 | | |
| NDUFS8 | 6 | 3′flanking + 693 | ttcacttcatttgcagtgag G/A aaaccagctccgagaggtga | 982 | | |
| NDUFS8 | 7 | 3′flanking + 1267 | ttttcccagacgtaaccgcc G/A tcagagcgtggcatggagcc | 983 | | |
| NDUFS8 | 8 | 3′flanking + 1362 | cgctgggttctttcccttac C/T gtggtctcccaggcacttac | 984 | | |
| NDUFS8 | 9 | 3′flanking + 1449 | tgtcagaacaggcctatggc G/A cccaaccacaagtcccccaa | 985 | | |
| NDUFS8 | 10 | 3′flanking + 1572 | cagccccacaggcctgtgct C/A gctgtgtggggcttagggat | 986 | | |
| NDUFS8 | 11 | 3′flanking + (783-784) | cagagaccttgacccccccc (G) atctaccatcatttccaaaa | 987 | | |
| NDUFS8 | 11 | 3′flanking + (783-784) | cagagaccttgacccccccc atctaccatcatttccaaaa | 988 | | |
| NDUFB3 | 1 | 5′flanking − 1439 | ttaaaagttgacttttttct G/A ccgggcacggtggctcacgc | 989 | | |
| NDUFB3 | 2 | 5′flanking − 1436 | aaagttgacttttttctgcc G/A ggcacggtggctcacgcctg | 990 | | |
| NDUFB5 | 1 | 5′flanking − 213 | ggcggatgaaactctcctac A/C aagaagggccaaaccggccg | 991 | | |
| NDUFB5 | 2 | intron 1 + 6288 | ggggatgttgattacctagg T/C cagtaaagtaaagaaggcat | 992 | | |
| NDUFB5 | 3 | intron 1 − 1581 | cttctgggccactgtatcct A/G tttctttcccttgttaccct | 993 | | |
| NDUFB5 | 4 | intron 1 − 1487 | ccctcttagaccgtatatag T/G tctagcataggatctgcaca | 994 | | |
| NDUFB5 | 5 | intron 2 + 556 | ttgtctggaccatctgccac G/A gtagataaagctctgaatca | 995 | | |
| NDUFB5 | 6 | intron 3 + 467 | ggcgccatcgcactccagcc C/T gggcaacagagtgagactct | 996 | | |
| NDUFB5 | 7 | intron 4 + 497 | agtgagactctgtccccccc C/G caaaaaaaaactataatcct | 997 | | |
| NDUFB5 | 8 | exon 5 + 397 | atgatagtcctgaaaagata T/C atgaaagaacaatggccgtc | 998 | | |
| NDUFB5 | 9 | intron 1 + (231-215) | attagcatttctaaaacgtt GTT/Δ attcaccatcccaattaatg | 999 | | |
| NDUFB7 | 1 | intron 1 + 68 | cctgaacacctggcacccca G/A ggctggcaccccagggctgg | 1000 | | |
| NDUFB7 | 2 | intron 2 + 266 | gggctctctaggggcctgtt T/C gatggggacagggcaggtgg | 1001 | | |
| ABCA1 | 1 | 5′flanking − 278 | gggcccgggcgggggaaggg G/C acgcagaccgcggaccctaa | 1002 | | |
| ABCA1 | 2 | 5′flanking − 99 | acataaacagaggccgggaa G/C ggggcggggaggagggagag | 1003 | | |
| ABCA1 | 3 | intron 1 + 159 | gcggtgttaaatggggagac G/T atgtcctagtacgagctctg | 1004 | | |
| ABCA1 | 4 | intron 1 + 506 | gaattggctatatgctcccc G/C ggactggagcggcacagtcc | 1005 | | |
| ABCA1 | 5 | intron 1 + 5897 | gtacaaaaccctttagcttt T.G gcaaacctcctttaagaccc | 1006 | | |
| ABCA1 | 6 | intron 1 + 5929 | ttaagacccgatttaaatgc C/T tccctcctcatgaagctctt | 1007 | | |
| ABCA1 | 7 | intron 1 + 5962 | aagctcttctggatccactc T/C ttcccatcactaagttgaaa | 1008 | | |
| ABCA1 | 8 | intron 1 + 5985 | cccatcactaagttgaaagt A/C agatccccttctctttactt | 1009 | | |
| ABCA1 | 9 | intron 1 + 11416 | ttacagtgccctttatagga G/A agaaagaagaaattgtgtct | 1010 | | |
| ABCA1 | 10 | intron 1 + 11935 | tctctgtggagcaaatagag G/A gctgtctgacacttggttcc | 1011 | | |
| ABCA1 | 11 | intron 1 + 12281 | gaatgtttgatttgtgaaaa T/A cttaataacagtagtttttt | 1012 | | |
| ABCA1 | 12 | intron 1 + 12924 | gtgctgacaatcttatactc T/C aggttgaacctccggggaag | 1013 | | |
| ABCA1 | 13 | intron 1 + 13002 | gagcctcaatcacagattct C/G tctagctcacatgaagttaa | 1014 | | |
| ABCA1 | 14 | intron 1 + 17715 | ggagcatgactttgtggaag C/T ctctcctcttccacccagag | 1015 | | |
| ABCA1 | 15 | intron 1 + 17848 | gagggctgactgtcaccctt T/C gataggagcccagcactaaa | 1016 | | |
| ABCA1 | 16 | intron 1 + 21384 | gtgggtgggaggaattggag G/C aggaagcttgcctaagtgtg | 1017 | | |
| ABCA1 | 17 | intron 1 + 22145 | gtagcttctaaatcaacgaa C/G tgattcctggagagcagctt | 1018 | | |
| ABCA1 | 18 | intron 1 + 23063 | ggaggcacctgtgaaaccaa G/A cggagtaggggggcggtgtg | 1019 | | |
| ABCA1 | 19 | intron 1 + 23131 | agtgtgcatatgtgctgacc G/A tgggagcttgtttgtcggtt | 1020 | | |
| ABCA1 | 20 | intron 2 + 156 | ggacacaggactgtgtggtc T/C ggatatggcatgtggcttat | 1021 | | |
| ABCA1 | 21 | intron 2 + 384 | gctgtgggtgaagtgagtta A/G tggccccactcttagagatc | 1022 | | |
| ABCA1 | 22 | intron 2 + 1081 | agtgcagccaaaattgcaaa G/A tcataccattcaaattaata | 1023 | | |
| ABCA1 | 23 | intron 2 + 2801 | aagaaaagtgatttatttca A/G gttgctgatgcttagattgt | 1024 | | |
| ABCA1 | 24 | intron 2 + 2830 | tgcttagattgttagagttg C/G aaagatctggcttgcatctt | 1025 | | |
| ABCA1 | 25 | intron 2 + 2856 | tctggcttgcatcttgtaca A/G ctgacagaactggggctcag | 1026 | | |
| ABCA1 | 26 | intron 2 + 3187 | tgatagctgttgcctgcagc A/G tacggacgttcattgcgcag | 1027 | | |
| ABCA1 | 27 | intron 2 + 3190 | tagctgttgcctgcagcata C/T ggacgttcattgcgcagttc | 1028 | | |
| ABCA1 | 28 | intron 2 + 3194 | tgttgcctgcagcatacgga C/T gttcattgcgcagttcctgt | 1029 | | |
| ABCA1 | 29 | intron 2 + 3204 | agcatacggacgttcattgc G/A cagttcctgtctcctgagat | 1030 | | |
| ABCA1 | 30 | intron 2 + 3401 | acataaagcctgtgtgctgc T/C gccaggaagactagaaacgc | 1031 | | |
| ABCA1 | 31 | intron 2 + 13927 | gtcaccacatacctggcact A/G tgctaaggctgggaatgcag | 1032 | | |
| ABCA1 | 32 | intron 3 + 4163 | ccagcccacttcatcttacc G/A tagttacctccttagagtat | 1033 | | |
| ABCA1 | 33 | intron 3 + 4262 | tgtcaaagaggaactaagga T/C gccagggactttctgcttag | 1034 | | |
| ABCA1 | 34 | intron 3 + 4306 | ccctctcatcacttctccaa C/T gctggtatcatgaaccccat | 1035 | | |
| ABCA1 | 35 | intron 5 + 240 | gacagaagaaaagtccccag G/A gaagaatactacagacttgg | 1036 | | |
| ABCA1 | 36 | intron 5 + 490 | gatgggcatttgaacttgtt G/A tctttaaaaagtgaaatctt | 1037 | | |
| ABCA1 | 37 | intron 5 + 583 | tatctggggagtgggcattt T/G ctgactgaggcattggctgc | 1038 | | |
| ABCA1 | 38 | intron 5 + 1051 | ggctacaaaactgtgctttc C/T ttgggcagtaaaagaggcaa | 1039 | | |
| ABCA1 | 39 | intron 5 + 3051 | tagagaacaagtctaattct G/A ttttccttgaaatagtcgaa | 1040 | | |
| ABCA1 | 40 | intron 5 + 3127 | aagtccatgattttttaggc A/G aaatggcctcctttcctctt | 1041 | | |
| ABCA1 | 41 | intron 5 + 5924 | ctttctttcacaaaattgcc C/T cccagagctttctggaaggg | 1042 | | |
| ABCA1 | 42 | intron 5 + 6831 | ccagtccctcagccttgcca T/C tgcttatgctggtctggaaa | 1043 | | |
| ABCA1 | 43 | intron 5 + 12678 | gctcaccgctctgctcaccc G/C accctctggccatctcctct | 1044 | | |
| ABCA1 | 44 | intron 5 + 14214 | cagcttggtcccagaggcct G/A gacctgggtcccagaggtcc | 1045 | | |
| ABCA1 | 45 | intron 5 + 14257 | gctggttccccggcttggtc C/T cagaggcctggatgtgtggc | 1046 | | |
| ABCA1 | 46 | intron 5 + 18078 | cctaccacaccatgcacgtg C/T acagccaagggttgttgact | 1047 | | |
| ABCA1 | 47 | intron 5 + 18795 | ctgggctcttcctggacctg G/A ccagctaaaaggaaatctcc | 1048 | | |
| ABCA1 | 48 | intron 5 + 18948 | gcattggtggtactaagaac G/A catattccctatcctatagg | 1049 | | |
| ABCA1 | 49 | intron 5 + 19053 | ctcccccaacattaaaagtg T/C aagggatgcttattcaaatg | 1050 | | |
| ABCA1 | 50 | intron 5 + 19148 | ggcccaagaaactgcatttt C/A gcatgctccctaaatgaagc | 1051 | | |
| ABCA1 | 51 | intron 5 + 19229 | atgctaacagtgtagagtca C/T atgtgatgggaagcatcagg | 1052 | | |
| ABCA1 | 52 | intron 5 + 19405 | cttgctcaatttattctgtc T/C atataactcaatattactga | 1053 | | |
| ABCA1 | 53 | intron 5 + 19534 | catgtgaccctcttagctcc G/A cggattaactcctgtcctca | 1054 | | |
| ABCA1 | 54 | exon 6 + 474 | gaaaccttctctgggttcct G/A tatcacaacctctctctccc | 1055 | | |
| ABCA1 | 55 | intron 6 + 210 | gcaacctggcgtcatgggcc A/G gctggttaaaataaaattga | 1056 | | |
| ABCA1 | 56 | intron 6 + 334 | acagttctgaggcaataacc G/A tggttaagggttattgatct | 1057 | | |
| ABCA1 | 57 | intron 6 + 2288 | cttctttcaaagcttgtggt C/T cactggaccacgtatgaagt | 1058 | | |
| ABCA1 | 58 | intron 6 + 2322 | atgaagtagaatagtttagg T/C ccagaaaggcaattaagtaa | 1059 | | |
| ABCA1 | 59 | intron 6 + 2820 | gtgctttgatacattctgag T/G ttcagtaaagagacctgatg | 1060 | | |
| ABCA1 | 60 | exon 7 + 656 | tgagctttgtggcctaccaa G/A ggagaaactggctgcagcag | 1061 | | |
| ABCA1 | 61 | intron 7 + 416 | catcataaagatgacattgt G/A ggctgtcacagttggaaggc | 1062 | | |
| ABCA1 | 62 | intron 7 + 471 | agaccacactatttagctta C/T ttagtaataacattgcaaag | 1063 | | |
| ABCA1 | 63 | intron 7 + 504 | ttgcaaagaaaaattccgac G/A aagttttttcagcctaggaa | 1064 | | |
| ABCA1 | 64 | intron 7 + 679 | gctctggtgaaattcctctc G/C ctaccccaaacatcatcatt | 1065 | | |
| ABCA1 | 65 | intron 7 + 1740 | acaaatgctcaccctttcag G/T tggaatgattgaaattttgg | 1066 | | |
| ABCA1 | 66 | intron 7 + 2122 | tgattaaggtggctactacc A/G ggtgctttctgcatatctcg | 1067 | | |
| ABCA1 | 67 | intron 7 + 7753 | taggaattccaagctgtgaa T/C tttttactgaagctctttgg | 1068 | | |
| ABCA1 | 68 | intron 7 + 8973 | atggaaatttgtttatattg A/T ctacagattgccaatattat | 1069 | | |
| ABCA1 | 69 | intron 7 + 8976 | gaaatttgtttatattgact A/G cagattgccaatattattag | 1070 | | |
| ABCA1 | 70 | intron 7 + 11327 | ctaacaatcttatttccatt G/C agtccttataaaagaagtgg | 1071 | | |
| ABCA1 | 71 | intron 7 + 11738 | ctgacgtttaagggagaccg C/T gtaggtccctttgaggactg | 1072 | | |
| ABCA1 | 72 | intron 7 + 12295 | agtctgtaaattattgttct T/A ttttttctttagcttatgct | 1073 | | |
| ABCA1 | 73 | intron 8 + 387 | tagcaaggccaatcatttta C/G caacacacatgcttgctaac | 1074 | | |
| ABCA1 | 74 | intron 8 + 697 | ggaactgtctggtgtccccc A/T gcataggaagctgagccagg | 1075 | | |
| ABCA1 | 75 | intron 8 + 1312 | attgctctgcagatcccctc G/A cagccctctgtcccttgttc | 1076 | | |
| ABCA1 | 76 | intron 8 + 3036 | ctttatgtgggaagaaattt T/G tttttttgattggggagtgg | 1077 | | |
| ABCA1 | 77 | intron 8 + 3176 | aaatggcctggttctctgtc C/A cctttctgtctgtatgcctc | 1078 | | |
| ABCA1 | 78 | intron 8 + 3364 | ggcagaaggcaaagcttagg A/T cctagagagtgctggaccac | 1079 | | |
| ABCA1 | 79 | intron 8 + 3373 | caaagcttaggacctagaga G/A tgctggaccacgccactcac | 1080 | | |
| ABCA1 | 80 | intron 8 + 3581 | cagggatttattaatgattt C/A ttgtgaaatgtttggaaata | 1081 | | |
| ABCA1 | 81 | intron 8 + 3654 | agtgccggaatacatttgca T/C gtaagacagaccgctgcctg | 1082 | | |
| ABCA1 | 82 | intron 8 + 4715 | ggcagaggggtctcagaatc C/T gcatttccaacaatgtctcc | 1083 | | |
| ABCA1 | 83 | exon 9 + 936 | cgtattgtctgcgggcatcc C/T gagggaggggggctgaagat | 1084 | | |
| ABCA1 | 84 | intron 9 + 2309 | cccctcaagagtcagtttaa A/G tgttggtcatgttagttgtc | 1085 | | |
| ABCA1 | 85 | intron 9 + 2392 | atgggagggcttgtgcttca T/C gaaaacatttttccagatca | 1086 | | |
| ABCA1 | 86 | intron 10 + 228 | tggggatggggaggactggc A/G cagggctgctgtgatggggt | 1087 | | |
| ABCA1 | 87 | intron 10 + 319 | ttctgcggtccctggctccc C/T acctgactccaggtgaacaa | 1088 | | |
| ABCA1 | 88 | intron 11 + 377 | gaaagaagtgtgggagcaaa A/C gcatgatgttacatgtagac | 1089 | | |
| ABCA1 | 89 | intron 11 + 521 | agtgctctagagacaattgg G/A ttcaaatgtggagcaggctg | 1090 | | |
| ABCA1 | 90 | intron 11 + 2850 | ctctatacaatcattatgct G/C ccattgaaataataaataca | 1091 | | |
| ABCA1 | 91 | intron 11 + 2976 | ctccaattcggtagaaccag A/G gcttcatcttctctgtcgaa | 1092 | | |
| ABCA1 | 92 | intron 11 + 3056 | gtttgcagctgctgtttttc G/T ggcagcacatctgtgcaggc | 1093 | | |
| ABCA1 | 93 | intron 12 + 340 | ggcattatttgtgaaactta T/C ctaaaatcgaattcgggtcc | 1094 | | |
| ABCA1 | 94 | intron 12 + 381 | aattaaatttttgaaatttt A/G tattaaaaattatattagta | 1095 | | |
| ABCA1 | 95 | intron 14 + 1728 | caggctcagaggccttggcc C/T atcaccctggctcacgtgtg | 1096 | | |
| ABCA1 | 96 | exon 15 + 2040 | atgggcctggacaacagcat C/A ctctggtttagctggttcat | 1097 | | |
| ABCA1 | 97 | intron 15 + 1382 | cttttagacagaaaagttac G/A tgggatattatctcccacag | 1098 | | |
| ABCA1 | 98 | intron 15 + 1453 | tatataaggagaaaccagtt G/A aaattacctattgaagaaac | 1099 | | |
| ABCA1 | 99 | intron 15 + 1567 | ttctgcgtagttttgggtaa G/A tcacttatcttctttaggat | 1100 | | |
| ABCA1 | 100 | intron 15 + 1617 | cagttgcctcatcagaaaga T/A gaacagcattacgcctctgc | 1101 | | |
| ABCA1 | 101 | intron 16 + 95 | agttgagaacagaagatgat T/A gtcttttccaatgggacatg | 1102 | | |
| ABCA1 | 102 | intron 16 + 452 | tggtgttttgcttgagtaat G/A ttttctgaactaagcacaac | 1103 | | |
| ABCA1 | 103 | intron 16 + 657 | ctgttgcctcagtctgggct T/C cataggcatcagcagcccca | 1104 | | |
| ABCA1 | 104 | exon 17 + 2473 | gcttcaatctcaccacttcg G/A tctccatgatgctgtttgac | 1105 | | |
| ABCA1 | 105 | exon 18 + 2649 | ggttccaaccagaagagaat A/G tcagaaagtaagtgctgttg | 1106 | | |
| ABCA1 | 106 | intron 18 + 1730 | tgaaagttcaagcgcagtgc C/G ctgtgtccttacactccact | 1107 | | |
| ABCA1 | 107 | intron 19 + 428 | aggaccttacagtgggtagt A/G tcaggaggggtcaggggctg | 1108 | | |
| ABCA1 | 108 | intron 19 + 468 | aaagcaccagcgttagcctc A/G gtggcttccagcacgattcc | 1109 | | |
| ABCA1 | 109 | intron 20 + 876 | ccctcctcatctaaagtgaa C/T acatggggctcatgtgcagg | 1110 | | |
| ABCA1 | 110 | intron 22 + 118 | catgggatactcttctgtta T/G cacagaagagataaagggca | 1111 | | |
| ABCA1 | 111 | intron 22 + 560 | aaagctttgccattctaggg G/A tcatagccatacagggtgaa | 1112 | | |
| ABCA1 | 112 | intron 23 + 102 | accccttttgccatgttgaa A/G ccaccatctccctgctctgt | 1113 | | |
| ABCA1 | 113 | intron 23 + 287 | gtcaaagaaaagagacttgt C/T gtttatggatggatagtcta | 1114 | | |
| ABCA1 | 114 | intron 23 + 1063 | acctttcaccctcaggaagc G/A aggctgttcacacggcacac | 1115 | | |
| ABCA1 | 115 | intron 25 + 321 | ctctttacttaagtacagtg T/G gaggaacagcggcatcagga | 1116 | | |
| ABCA1 | 116 | intron 25 + 376 | gttagaaattcagcaacttg G/C gcccagctcagacctactga | 1117 | | |
| ABCA1 | 117 | intron 25 + 478 | catacataggaaatgacaaa C/T gtttatggatggatagtcta | 1118 | | |
| ABCA1 | 118 | intron 25 + 579 | tcatttaattctcaaaaaaa G/T atgaaaaaatgaacactcag | 1119 | | |
| ABCA1 | 119 | intron 27 + 153 | aatggtaaaagccacttgtt C/T tttgcagcatcgtgcatgtg | 1120 | | |
| ABCA1 | 120 | intron 28 + 1058 | actatcatgggagataatga C/T tatggttgtccatgattgga | 1121 | | |
| ABCA1 | 121 | intron 28 + 1317 | caggacccagtgttctgagt C/T accctgaatgtgagcactat | 1122 | | |
| ABCA1 | 122 | intron 30 + 372 | tatatgatttttaggttttg T/C ttatcagcttcttcgctttt | 1123 | | |
| ABCA1 | 123 | intron 30 + 506 | ccttttaaaaagtaagcagt A/G gataaataaattcagtgaag | 1124 | | |
| ABCA1 | 124 | intron 30 + 1033 | ctggatttcatggtgccttt G/c attttccacatgaaggttgt | 1125 | | |
| ABCA1 | 125 | exon 31 + 4281 | tcttccctttgcagagacac G/A ccctgccaggcaggggagga | 1126 | | |
| ABCA1 | 126 | intron 33 + 626 | ggctccttgttactgatttc C/T gtcttttctctctgcctttt | 1127 | | |
| ABCA1 | 127 | intron 33 + 719 | taatagccctcatgctagaa G/A ggagccggagcctgtgtata | 1128 | | |
| ABCA1 | 128 | intron 33 + 726 | cctcatgctagaagggagcc G/A gagcctgtgtataaggccag | 1129 | | |
| ABCA1 | 129 | intron 33 + 889 | ctttcctcaatgtctcagct A/G tctaactgtgtgtgtaatca | 1130 | | |
| ABCA1 | 130 | intron 33 + 1097 | ctgtgcaccccactgtctgg G/C ttttaatgtcaggctgttct | 1131 | | |
| ABCA1 | 131 | intron 35 + 4760 | tatgacaggactggacacca G/A aaataatgtcaaggtaaacc | 1132 | | |
| ABCA1 | 132 | intron 35 + 234 | aacctatctaaacctcagtt T/C cctcatctgtgaaatggaga | 1133 | | |
| ABCA1 | 133 | intron 37 + 411 | aactctgtacattttatcag C/T agcttatccatccattgcaa | 1134 | | |
| ABCA1 | 134 | intron 37 + 1224 | caggcataggtgattcagag A/G tgaaaggtcaagtccctgaa | 1135 | | |
| ABCA1 | 135 | intron 37 + 1720 | aaattaaaattactctgact G/T ggaatccatcgttcagtaag | 1136 | | |
| ABCA1 | 136 | intron 40 + 251 | tgaaggtaaggaaaatagtg T/G tatttgcttggatccactgg | 1137 | | |
| ABCA1 | 137 | intron 40 + 252 | gaaggtaaggaaaatagtgt T/C atttgcttggatccactggc | 1138 | | |
| ABCA1 | 138 | intron 40 + 319 | agcactggaaaagtcaaacc A/G taactttgagaattaggtga | 1139 | | |
| ABCA1 | 139 | intron 40 + 957 | cttgttactcttttttcctt G/C tcatgggtgatagccatttg | 1140 | | |
| ABCA1 | 140 | intron 41 + 146 | tgatgtgggcatcccgcagc C/T ccctccctgcccatcctgga | 1141 | | |
| ABCA1 | 141 | intron 42 + 238 | cattggttttatatgcttac A/C tttatgtgttagttattaaa | 1142 | | |
| ABCA1 | 142 | intron 42 + 321 | aataaatggttgattttgag T/A ttgagtttcatagtccaaaa | 1143 | | |
| ABCA1 | 143 | intron 42 + 322 | ataaatggttgattttgagt T/C tgagtttcatagtccaaaaa | 1144 | | |
| ABCA1 | 144 | intron 42 + 533 | agatgaaaaattatgtagat G/A ataatgaatgatacggttct | 1145 | | |
| ABCA1 | 145 | intron 42 + 546 | tgtagatgataatgaatgat A/G cggttctaaaaagacaggtt | 1146 | | |
| ABCA1 | 146 | intron 43 + 739 | tacagccacacttaaaatgg T/A cccattatgaaatacatatt | 1147 | | |
| ABCA1 | 147 | intron 44 + 18 | taggtgagaaaagaagtggc T/C tgtattttgctgcaaagact | 1148 | | |
| ABCA1 | 148 | intron 44 + 264 | acaatataatttgcttgttt T/C ttaagagtataatttagtga | 1149 | | |
| ABCA1 | 149 | intron 44 + 279 | tgttttttaagagtataatt T/C agtgatttttggtaaattga | 1150 | | |
| ABCA1 | 150 | intron 44 + 508 | tttacattgctacataaaat C/T cccctatgtacatgtaccta | 1151 | | |
| ABCA1 | 151 | intron 44 + 1477 | gatctcctctcctgtctctt A/T catttttgcagtagcaatgt | 1152 | | |
| ABCA1 | 152 | intron 44 + 1665 | tggttgtaagaactgatttg G/A ttggtatagctgtgagggcc | 1153 | | |
| ABCA1 | 153 | intron 44 + 1956 | gtgttgctcacactcaaaat T/G tctgggccttctcatttggt | 1154 | | |
| ABCA1 | 154 | intron 45 + 68 | aatatataccttatggcttt T/C ccacacgcattgacttcagg | 1155 | | |
| ABCA1 | 155 | intron 46 + 608 | ttatactgacttcaatagag G/C tttcagacaaaaagttgttt | 1156 | | |
| ABCA1 | 156 | intron 47 + 336 | ttcacaattgtaaacaccac T/C acactgaacagcatcatccc | 1157 | | |
| ABCA1 | 157 | intron 49 + 55 | agggtgtggattcctgcccc G/C acactcccgcccataggtcc | 1158 | | |
| ABCA1 | 158 | 3′UTR(exon 50)+ 7949 | aacaaaaatgtgggtgtctc C/T aggcacgggaaacttggttc | 1159 | | |
| ABCA1 | 159 | 3′UTR(exon 50)+ 8226 | aggagcccactgtaacaata C/T tgggcagccttttttttttt | 1160 | | |
| ABCA1 | 160 | 3′UTR(exon 50)+ 8682 | aacttcttccactttttcca G/A aatttgaatattaacgctaa | 1161 | | |
| ABCA1 | 161 | 3′UTR(exon 50)+ 8697 | ttccagaatttgaatattaa C/T gctaaaggtgtaagacttca | 1162 | | |
| ABCA1 | 162 | 3′UTR(exon 50)+ 9097 | aactattttgaagaaaacac A/G acattttaatacagattgaa | 1163 | | |
| ABCA1 | 163 | 5′flanking − (1033-1032) | tgacttaaatatttagacat (AT) ggtgtgtaggcctgcattcc | 1164 | | |
| ABCA1 | 163 | 5′flanking − (1033-1032) | tgacttaaatatttagacat ggtgtgtaggcctgcattcc | 1165 | | |
| ABCA1 | 164 | intron 5 + 6368 | ttctgatggggttgttgctg C/Δ tgagaatcatgactgggtgg | 1166 | | |
| ABCA1 | 165 | intron 5 + 9709 | cattttctgtctgaaccccc T/Δ cacccattcaggcagctgct | 1167 | | |
| ABCA1 | 166 | intron 5 + 13816 | tccctacttctccttttttt T/Δ catttgcctcctccacccac | 1168 | | |
| ABCA1 | 167 | intron 10 + (270-271) | cttttcagggaggagccaaa (G) cgctcattgtctgtgcttct | 1169 | | |
| ABCA1 | 167 | intron 10 + (270-271) | cttttcagggaggagccaaa cgctcattgtctgtgcttct | 1170 | | |
| ABCA1 | 168 | intron 20 + (611-612) | tttagcccatcctctccccc (C) gccaccctccttattgaggc | 1171 | | |
| ABCA1 | 168 | intron 20 + (611-612) | tttagcccatcctctccccc gccaccctccttattgaggc | 1172 | | |
| ABCA1 | 169 | intron 32 + (391-392) | gagtgccttgggtactctct (T) gatgggggactccatgataa | 1173 | | |
| ABCA1 | 169 | intron 32 + (391-392) | gagtgccttgggtactctct gatgggggactccatgataa | 1174 | | |
| ABCA1 | 170 | intron 37 + 847 | gctgtatattgtgaatgtcc C/Δ gttttcaaaagcaaaagccaa | 1175 | | |
| COMT | 1 | 5′flanking − 1287 | cgtatgatattccccattct G/A agtccagaatacctagaaat | 1176 | | |
| COMT | 2 | 5′flanking − 1217 | tgtgagtatgggaaggggaa G/A cttttctgtctgttgtcccc | 1177 | | |
| COMT | 3 | 5′flanking − 503 | caggggctccaggaggacga G/A tgtgtatcctcccattgctc | 1178 | | |
| COMT | 4 | 5′flanking − 425 | gagaagttgggaagtctggc C/T agtggggccggtgcctggtg | 1179 | | |
| COMT | 5 | 5′flanking − 277 | cccagccccagtttccccac C/T tgggaagggggctacttgtg | 1180 | | |
| COMT | 6 | intron1 + 12058 | ctggcccatggaagggaggg G/A agggggccccgacggggcca | 1181 | | |
| COMT | 7 | intron1 + 12070 | agggaggggagggggccccg A/G cggggccacagtaaaggagt | 1182 | | |
| COMT | 8 | intron1 + 18831 | tgtgtatgttcttggtaaac C/T agcccttggtcttacacatc | 1183 | | |
| COMT | 9 | intron2 + 832 | cctctcctttggccacccgt G/C actacccccaactccgggcc | 1184 | | |
| COMT | 10 | intron3 + 90 | ggagaagctgttatcacccc A/G tttccagggggctgggaacc | 1185 | | |
| COMT | 11 | intron3 + 425 | ccccaaggtgggcggttcgg T/G gattcagagagggcagctct | 1186 | | |
| COMT | 12 | intron3 + 871 | ggctcctgctctttgggaga G/A gtggggggccgtgcctgggg | 1187 | | |
| COMT | 13 | intron3 + 876 | ctgctctttgggagaggtgg G/T gggccgtgcctggggatcca | 1188 | | |
| COMT | 14 | intron4 + 75 | tcagcctcagcctctccaaa G/C agccaggcattccagtagag | 1189 | | |
| COMT | 15 | intron5 + 310 | accagacaccagggcagaaa C/T ggcacaggaccaaggagatg | 1190 | | |
| COMT | 16 | intron5 + 346 | agatggggtggggaagggcc G/A ctctgggcccagcctgctct | 1191 | | |
| COMT | 17 | intron5 + 3023 | aaggcagccgccctgctcaa G/A gcctaggccattgtcctcct | 1192 | | |
| HNMT | 1 | 5′flanking − 211 | cagaggcagatgacagtctt C/T cgttaaagatttcactgctg | 1193 | | |
| HNMT | 2 | intron1 + 5409 | aatataactgatataattgg A/G acatttcatgttggcctagt | 1194 | | |
| HNMT | 3 | intron2 + 2561 | cacttgtgcttggacaagaa A/G agaaggcctacaagaaaaag | 1195 | | |
| HNMT | 4 | intron2 + 2895 | caatcagaaatgtaagaaaa A/C ctccaagaaaaatttaagtt | 1196 | | |
| HNMT | 5 | intron2 + 3977 | accaaacttggaagtgtaaa G/A ttatgcatgtatgttcatgt | 1197 | | |
| HNMT | 6 | intron2 + 5296 | ttaacatagtgagtttggag T/C cccaggattttattttcctt | 1198 | | |
| HNMT | 7 | intron2 + 13317 | caaccctcatgaattcttag C/T tgggatgggtccctataaca | 1199 | | |
| HNMT | 8 | intron2 + 14682 | gtagatgagcaaatgagttc A/Δ ggagagatttaaatacccta | 1200 | | |
| HNMT | 9 | intron2 + 15406 | gtctatgcattcatgcatcc G/A tctaaccagctgtctaccta | 1201 | | |
| HNMT | 10 | intron2 + 28943 | atgtgacttaaacttcaggt A/G tatcaatatcccttgaatgt | 1202 | | |
| HNMT | 11 | intron4 + 49 | cagaaagaagacttttcaga A/G tatatatataatgaatatct | 1203 | | |
| HNMT | 12 | intron4 + (1942-1943) | tttgagaaaaatttaaggta (A) tcttctatggcccacttcca | 1204 | | |
| HNMT | 12 | intron4 + (1942-1943) | tttgagaaaaatttaaggta tcttctatggcccacttcca | 1205 | | |
| HNMT | 13 | intron4 + 2405 | ccctgtgaccaagcagataa C/A ctcatgctttatttagtcca | 1206 | | |
| HNMT | 14 | intron5 + (80 − 81) | cctgtgtttgaaagaagctt (TT) atatattttgtcttcattat | 1207 | | |
| HNMT | 14 | intron5 + (80 − 81) | cctgtgtttgaaagaagctt atatattttgtcttcattat | 1208 | | |
| HNMT | 15 | intron5 + 235 | ctttcttttgggaaaatatg T/C ctttgtcttctatatatgaa | 1209 | | |
| HNMT | 16 | intron5 + (702-703) | tacttacaggttgattttag (AT) acacagcagactctgtcttc | 1210 | | |
| HNMT | 16 | intron5 + (702-703) | tacttacaggttgattttag acacagcagactctgtcttc | 1211 | | |
| HNMT | 17 | intron5 + 749 | ttacaccagaccccatactt T/G aacaccatatgtcacaaaat | 1212 | | |
| HNMT | 18 | intron5 + 1101 | gtaggcagcctattcttgat T/G atattcatcaatcatacaga | 1213 | | |
| HNMT | 19 | intron5 + 1137 | acagaaaaagtattgtagac G/A gaaataacaattcattgaga | 1214 | | |
| HNMT | 20 | intron5 + 1348 | aagggagcatgaatagtcca C/G aagtaactgagaactgatta | 1215 | | |
| HNMT | 21 | intron5 + 1673 | caaaagaaagggagtaaaga C/G tcaacaatcagttagctttt | 1216 | | |
| HNMT | 22 | intron5 + 2022 | attttatttggggctttcta C/T gtctctctctcctaagccta | 1217 | | |
| HNMT | 23 | intron5 + 2285 | tgtcatacttaactcttaaa G/C atccagagtaaatgatggag | 1218 | | |
| HNMT | 24 | intron5 + 4159 | taccagttgacccagcaacc C/T tcttatagagtagtttaaat | 1219 | | |
| HNMT | 25 | intron5 + 4501 | aatgatccacaaaattacta C/G tcattgttttctttcaatga | 1220 | | |
| HNMT | 26 | intron5 + 5251 | cacacacacacacacacaca C/G caaatggaagcagccagaca | 1221 | | |
| HNMT | 27 | intron5 + 5802 | gaaaaagaaaatctggctta C/T atcatgttgaaaacaaaagt | 1222 | | |
| HNMT | 28 | intron5 + | 6189 tccaattccaccttctccta G/C agcatatcctgcagttacct | 1223 | | |
| HNMT | 29 | intron5 + 6297 | gtcttggttcatctcttgag T/A taaattagatctgggaactt | 1224 | | |
| HNMT | 30 | 3′flanking + 458 | tatgtcactctcaagaactc C/T tataagaccaagagtcatct | 1225 | | |
| HNMT | 31 | 3′flanking + 993 | ctgaaaatgaacactgaacc G/A ttaatcatactgatatgtac | 1226 | | |
| HNMT | 32 | 3′flanking + 1793 | gtggagcacagcattttagg G/A cttgatatttgcttattata | 1227 | | |
| GAMT | 3 | intron5 + 1411 | ggtgacctggtgccatcccc G/A accaggagacgcaggtgccc | 1228 | | |
| PNMT | 2 | intron1 + 35 | ctgaggcacgagggacaaga G/T gtcgtcggggagtgaaagca | 1229 | | |
| CYP1A1 | 1 | intron 1 + 1590 | ccactcttcaaaaggaggta C/T atgtgacagcagctggaaat | 1230 | | |
| CYP1A1 | 2 | exon2 + 160 | gaatccaccagggccatggg G/A ctggcctctgattgggcaca | 1231 | | |
| CYP1A2 | 1 | 5′flanking − 731 | gcctgggctaggtgtagggg T/G cctgagttccgggctttgct | 1232 | | |
| CYP1A2 | 2 | intron1 + 371 | cttccctgtgttcacactaa C/T cttttccttctttgaaattg | 1233 | | |
| CYP1A2 | 3 | intron3 + 44 | atagccaggagaagccttga G/A acccaggttgtttgttcagt | 1234 | | |
| CYP1A2 | 4 | intron5 + 81 | tccctgctaggaactgttta T/C ataatgaaaggaggggacct | 1235 | | |
| CYP1A2 | 5 | exon6 + 181 | ctggccatcctgctacagca A/T ctggagttcagcgtgccgcc | 1236 | | |
| CYP1A2 | 6 | exon6 + 295 | cggctgcgcttctccatcaa C/T tgaagaagacaccaccattc | 1237 | | |
| CYP1B1 | 1 | 5′flanking − 3669 | tgtatcctgtgaagcatcac G/A gttatccttctctgcacatg | 1238 | | |
| CYP1B1 | 2 | 5′flanking − 3149 | tgacagcacttaccaaccta G/C ttcctctgatttttgagtca | 1239 | | |
| CYP1B1 | 3 | 5′flanking − 1222 | gggggaagccacccccgccc G/A agcgcctccggcttccctta | 1240 | | |
| CYP1B1 | 4 | 5′flanking − 376 | ttccgggaagcaagctcaag T/C cgcggagagggaagggaggt | 1241 | | |
| CYP1B1 | 5 | 5′flanking − 265 | ctggggacaccgtgcggcct C/T gattggaggtggctgtgatg | 1242 | | |
| CYP1B1 | 6 | intron1 + 129 | tgcccgcagcgttgtcccca G/A attgcaggaaccgttacgcg | 1243 | | |
| CYP1B1 | 7 | intron1 + 379 | tgagtgtcacgccttctcct C/T tctgtccccagcatgggcac | 1244 | | |
| CYP1B1 | 8 | exon3 + (799-800) | agcttctgggagattttttt (T) gagtcaaagacttaaagggc | 1245 | | |
| CYP1B1 | 8 | exon3 + (799-800) | agcttctgggagattttttt gagtcaaagacttaaagggc | 1246 | | |
| CYP1B1 | 9 | exon3 + 1284 | agtatagtggggttccatga G/T ttatcatgaattttaaagta | 1247 | | |
| CYP1B1 | 10 | 3′flanking + 2226 | tttctttttctttttttttt T/Δ aaaatttattcctatttcct | 1248 | | |
| CYP1B1 | 11 | 3′flanking + (2226-2227) | ttcttttctttttttttt (T) aaaatttattcctatttcct | 1249 | | |
| CYP1B1 | 11 | 3′flanking + (2226-2227) | ttcttttctttttttttt aaaatttattcctatttcct | 1250 | | |
| CYP1B1 | 12 | 3′flanking + 2230 | ttttcttttttttttttaaa A/Δ tttattcctatttccttaca | 1251 | | |
| PEMT | 90 | intron1 + (297-298) | attgtgtgagactcagaggt TGT/Δ ccgtgttagtctttgggatt | 1252 | | |
| PEMT | 91 | intron1 + 817 | tcatgaagcctgtaaggcac A/G tctctgccccaagcagcttc | 1253 | | |
| PEMT | 92 | intron1 + 830 | aaggcacatctctgccccaa G/A cagcttctaatccagttctt | 1254 | | |
| PEMT | 93 | intron1 + 1035 | gagttctctgaaggagctaa T/C accagttagtgttttgaaga | 1255 | | |
| PEMT | 94 | intron1 + 1573 | agtgggcaggggagactaac C/T gggtgtgtgaggggtgggct | 1256 | | |
| PEMT | 95 | intron1 + 1759 | gatttttcttaaagaaagaa A/G gaaagaaacatacaacatac | 1257 | | |
| PEMT | 96 | intron1 + 2768 | gcatcttgctgtccacaggc C/A ggggcacctccaggattcag | 1258 | | |
| PEMT | 97 | intron1 + 2768 | ggccggggcacctccaggat T/C cagaagatgactccagtagg | 1259 | | |
| PEMT | 98 | intron2 + 4598 | ccgtgggttttttttttttt T/Δ cttcatttctttggttgctg | 1260 | | |
| NAT2 | 21 | exon2 + 288 | atgttaggagggtattttta C/T atccctccagttaacaaata | 1261 | | |
| NAT2 | 22 | 5′flank − 2053 | ctggattgcaacattttaat T/C ccaggtgtcaggtttccaac | 1262 | | |
| NAT2 | 23 | 5′flank − 1299 | gaatcaccagtgcgggaggt A/G taacagtgaacccaagacac | 1263 | | |
| NAT2 | 24 | 5′flank − 1145 | ctgtagaacacaaggatatt C/T ggaggcagtttgtacatgcc | 1264 | | |
| NAT2 | 25 | 5′flank − 1036 | ccttcccacagagtcccgag T/A tcatgtggcagcatgccaga | 1265 | | |
| NAT2 | 26 | 5′flank − 94 | aaagatttgctaagagattc G/A cagaggcaacctgaggccct | 1266 | | |
| NAT2 | 27 | 5′flank − 643 | atgtttatattttatattaa T/C attaatgtaaataaaaattt | 1267 | | |
| AADA | 1 | 5′UTR + 29 | attaaagtacactattcagg C/T atatcatgtaggtttacttt | 1268 | | |
| AADA | 2 | intron1 + 138 | gctgtggcctttgacaatgt G/A ttacttagaaatgttgtttg | 1269 | | |
| AADA | 3 | intron1 + 142 | tggcctttgacaatgtgtta C/T ttagaaatgttgtttgtttt | 1270 | | |
| AADA | 4 | intron1 + 1033 | ttccagcagagacaccaaca A/G gtaaaaacaccccagctaca | 1271 | | |
| AADA | 5 | intron1 + 1253 | tttttttccctcatatttgc T/C gtctgtgctacaatatgtga | 1272 | | |
| AADA | 6 | intron1 + 1366 | ctctggtcgccttttaatta A/G ttaattcattcatttactta | 1273 | | |
| AADA | 7 | intron1 + 1369 | tggtagccttttaattaatt A/C attcattcatttacttacat | 1274 | | |
| AADA | 8 | intron1 + 2501 | ggttacagaaagaatggtgg C/A ttggccaaaaaatgatatgg | 1275 | | |
| AADA | 9 | intron2 + 1971 | aaatgagagttaagtaggag A/C attttcttttatttttgtgc | 1276 | | |
| AADA | 10 | intron2 + 1988 | gagaattttcttttattttt A/G tgcaggagaaatataaacaa | 1277 | | |
| AADA | 11 | intron2 + 2341 | aggtgccttttctattgtcc C/T atgcagacttaggtgatcct | 1278 | | |
| AADA | 12 | intron2 + 2546 | gtctgacacagaaggatcaa T/A ggcaaaatgtgcaagacaaa | 1279 | | |
| AADA | 13 | intron2 + 2609 | taggaggttcactgggaaac T/C tgaattccactgagtcatga | 1280 | | |
| AADA | 14 | intron2 + 2663 | tataaatacagtgttaaatt T/C gtctctcgtattttaaggta | 1281 | | |
| AADA | 15 | intron4 + 605 | tgtgtcagtaaaatattata T/C taagtaggtgaatgagatca | 1282 | | |
| AADA | 16 | intron4 + 621 | tatattaagtaggtgaatga G/T atcatgtaattgtgagacta | 1283 | | |
| AADA | 17 | intron4 + 679 | ttagagattcagacgaattc A/G tataatcttcgatggtgtat | 1284 | | |
| AADA | 18 | intron4 + 1680 | gttaaaatgtggataaatac C/T acaatttgcaaaatatttgg | 1285 | | |
| AADA | 19 | intron4 + 1748 | atttagaagttctatacatc T/C tttatagtatattacacact | 1286 | | |
| AADA | 20 | intron4 + 1771 | tatagtatattacacacttc G/A aaaacacaaaattatttttt | 1287 | | |
| AADA | 21 | exon5 + 238 | caagtcatctcttcaaattt A/G ttaattggagttccctgctc | 1288 | | |
| AADA | 22 | 3′UTR + 121 | ttagaaattggtctttctta A/G aatggtctagttaagttcca | 1289 | | |
| NTE | 1 | 5′flanking − 535 | cacgatctgtcctccgattc C/T tgttaactctagactttctg | 1290 | | |
| NTE | 2 | 5′flanking − 15 | gtaaatccccggcaaaaacc A/G gcagcgccttgcaagcccac | 1291 | | |
| NTE | 3 | 5′flanking − 748 | agcatggcgcggggaggagg G/T gtgggagggtcgggagggac | 1292 | | |
| NTE | 4 | 5′flanking − 690 | tgaataatttaaaggggccg T/C gcctgcggagccgggcggaa | 1293 | | |
| NTE | 5 | intron6 + 605 | tcttgccatatacttagtgg A/G ggggtctacatcaggggttt | 1294 | | |
| NTE | 6 | intron6 + 748 | agcctccagcctctcttctc C/T gggggttatctcaggcatct | 1295 | | |
| NTE | 7 | intron6 + 987 | ggtgctggctctgggatccc C/T gtgcgtcatgtagtctacct | 1296 | | |
| NTE | 8 | intron6 + 1882 | tggcctcaagcaatcctccc G/A cctcggcctcccaaagtgct | 1297 | | |
| NTE | 9 | intron6 + 2222 | gaatgtttatgtagaacaga G/A agactgtatctgcggtcttc | 1298 | | |
| NTE | 10 | intron12 + 168 | tatctggtaccgaggaagct C/G tggcctcgtccccaagggcc | 1299 | | |
| NTE | 11 | intron13 + 69 | atccaggtccaccgcctgcc C/T gtcttgattgttttaatctg | 1300 | | |
| NTE | 12 | intron14 + 8 | agcccccgctcgggtaaggc C/T tgggaccctgcccggtggtg | 1301 | | |
| NTE | 13 | intron16 − 113 | gccaccgcgccctgcgcctt T/C atatttttcttaacccttcc | 1302 | | |
| NTE | 14 | intron21 + 34 | agagccggccggcccagagc A/G tgctgggagatgtagtccgg | 1303 | | |
| NTE | 15 | intron21 + 128 | gaagaaatcgtgcccctgag G/A gtttcaaaccctaagtagga | 1304 | | |
| NTE | 16 | intron21 + 151 | ttcaaaccctaagtaggacc C/G aggtgcagagcattctgggg | 1305 | | |
| NTE | 17 | intron21 + 651 | ccactgtactccagccggga C/T gacagagctagaacctgttt | 1306 | | |
| NTE | 18 | intron21 + 797 | tggaaaatagtctgtggatt G/T ttgtttaggactctgggcac | 1307 | | |
| NTE | 19 | intron21 + 1752 | acagctggtctaggctgtta G/C tggagaaactgggaagcaac | 1308 | | |
| NTE | 20 | intron21 + 1788 | gaagcaacagctgggtcaaa A/Δ gtagcttttcttttcttggc | 1309 | | |
| NTE | 21 | intron21 + 1907 | cactgcaacctctgcctccc A/G ggttcaagtgattctcctgc | 1310 | | |
| NTE | 22 | intron21 + 2065 | ctgcctcgttttatgttcag G/T tcccccattagacagaggaa | 1311 | | |
| NTE | 23 | intron21 + 2336 | agtctgggagcacaggagca G/A gaatttcagataaggaggaa | 1312 | | |
| NTE | 24 | intron23 + 41 | tggggagggtggtgggtggg G/C ctggagcctcaaattctttc | 1313 | | |
| NTE | 25 | intron23 + 71 | caaattctttcagacctgag T/C tcaagttctcggcttccaac | 1314 | | |
| NTE | 26 | intron23 + 81 | cagacctgagttcaagttct C/T ggcttccaaccacggagcct | 1315 | | |
| NTE | 27 | intron24 + 150 | gtggggcggctggtgacctc A/C gccgtccgtattccgcagct | 1316 | | |
| NTE | 28 | intron29 + 37 | gcctgcagcaaccgctgacg T/C cacgtggggttggggggatg | 1317 | | |
| NTE | 29 | intron29 + 370 | cgtcccaggtcagcgagccc G/A tcgggccggctgggcctccg | 1318 | | |
| NTE | 30 | intron30 + 56 | acctcccgcaccacacacac G/A cacacgcgtgggcacacaca | 1319 | | |
| NTE | 31 | intron30 + 358 | aaaaatacaaaaaattaacc A/G ggctggtggggtgtgcctgt | 1320 | | |
| NTE | 32 | intron30 + 372 | ttaaccaggctggtggggtg T/C gcctgtaatcccagctactc | 1321 | | |
| NTE | 33 | intron30 + 430 | aaatcacttgaacctgggag G/T tggaggttgcagtgagctga | 1322 | | |
| NTE | 34 | intron30 + 655 | gtgtgcacaccagctatata T/C gcaaatgctttctctcaggg | 1323 | | |
| NTE | 35 | intron30 + 659 | gcacaccagctatatatgca A/C atgctttctctcaggggcag | 1324 | | |
| NTE | 36 | intron30 + 760 | tgaaatagggcatttgccaa C/T gcatgccagtctgtcccgtt | 1325 | | |
| NTE | 37 | intron30 + 835 | gcacacacgtagataggatg T/C ggcacctctgaccgagttaa | 1326 | | |
| NTE | 38 | intron31 + 40 | tggtgcctgcataggtggtc T/C ggctaagctttgctacttaa | 1327 | | |
| NTE | 39 | intron31 + 41 | ggtgcctgcataggtggtct G/A gctaagctttgctacttaaa | 1328 | | |
| NTE | 40 | intron31 + 1329 | gtctgtcaagggcaggacag G/A ggatgtgtaggcgagtgtgc | 1329 | | |
| NTE | 41 | intron35 + 31 | aatggcttcctgtcgttttc G/A gactggggacccaccttctg | 1330 | | |
| DDOST | 8 | intron2 + 1299 | atcttctgatgactgggctt C/T ggtgcagtaactggtgtttg | 1331 | | |
| DDOST | 9 | intron2 + 1581 | gatactgttggtgggagaaa T/C gacagagagtgtaaaacagt | 1332 | | |
| DDOST | 10 | intron2 + 2822 | gtttctcaacaggtgcattc T/G tgacgtttcagactggataa | 1333 | | |
| DDOST | 11 | intron2 + 3392 | cagaaggcgtggaggcctgc C/T gcgcctccctctgttgctgc | 1334 | | |
| DDOST | 12 | intron2 + 495 | attgcttgaacccaggaggc G/A gaggttgcagtgagccaagg | 1335 | | |
| DDOST | 13 | intron5 + 226 | ggaactgcttgggtcacagc C/T tcgttttgttcccagtatcc | 1336 | | |
| DDOST | 14 | intron8 + 303 | aagagaaataggtcattagg A/T tgaatttgttaggcaagaga | 1337 | | |
| DDOST | 15 | 3′flanking + 40 | cacagcgtggagacggggca G/A ggaggggggttattaggatt | 1338 | | |
| MRP2 | 1 | exon 1 + 77 | catattaatagaagagtctt G/C gttccagacgcagtccagga | 1339 | | |
| MRP2 | 2 | intron2 + 192 | atcaaagtggctttgatttt T/G gcataagaatggtgactctt | 1340 | | |
| MRP2 | 3 | intron 1 + 413 | gataagttctagaactggca A/C ctaatgatatggactagaag | 1341 | | |
| MRP2 | 4 | intron2 + 3639 | gtcatatcccacccccaaat C/A gacccaataggtacaatgaa | 1342 | | |
| MRP2 | 5 | intron2 + 3989 | agttatgaaaccgatttttc C/T gggactggttgttctagtct | 1343 | | |
| MRP2 | 6 | intron2 + 4078 | aggtttccagatgtgttccc T/C aggcattcctggtggtagga | 1344 | | |
| MRP2 | 7 | intron2 + 4171 | cttattctttggtcagttgg C/T tttctaccacctcttagctt | 1345 | | |
| MRP2 | 8 | intron 2 + 5373 | gttaaggatatgtgaactca A/G aatttttatacacagtgcaa | 1346 | | |
| MRP2 | 9 | intron2 + 4436 | ggactagtggaagaattaga C/G ctttcctgaataaatagatc | 1347 | | |
| MRP2 | 10 | intron 2 + 3930 | aaaactggcaggagaatttc A/G ctggagctgcatgcaggact | 1348 | | |
| MRP2 | 11 | intron 2 + 4257 | gggtattggaaagttcttgc G/A gctgctggaggctgcggtgt | 1349 | | |
| MRP2 | 12 | intron 3 + 772 | ggtataaggcaagatttttt A/T aaaaaattaattgcttaatc | 1350 | | |
| MRP2 | 13 | intron 7 + 1658 | ggactcttaccagcttagtt G/T cctggttttctaatctaaaa | 1351 | | |
| MRP2 | 14 | intron 10 + 40 | tggccaggaaggagtacacc G/A ttggagaaacagtgaacctg | 1352 | | |
| MRP2 | 15 | intron 11 + 1672 | aactttttaagtcttaagac T/A ggaaggcctgtgtcctaggc | 1353 | | |
| MRP2 | 16 | intron 12 + 148 | ccctctcaccgccccatgcc A/G cttttcctcctttgtaccat | 1354 | | |
| MRP2 | 17 | intron 2 + 1020 | agtgctgcgattacaagcct G/C agccacctgcacagcctctg | 1355 | | |
| MRP2 | 18 | intron 2 + 5227 | taccataatttatgtgtcct A/G tatgacatgaatttcattgg | 1356 | | |
| MRP2 | 19 | intron 2 + 5373 | gttaaggatatgtgaactca A/G aatttttatacacagtgcaa | 1357 | | |
| MRP2 | 20 | intron 2 + 5538 | ttaatgaggttaagcacatg G/T tcatatgtttaaaagccttt | 1358 | | |
| MRP2 | 21 | intron 13 + 180 | catgagttttctgagcccca G/C tttatctaactataaaatga | 1359 | | |
| MRP2 | 22 | intron 13 + 1497 | gtgcagggtccccctgatgc T/C atagccagttcctctttaga | 1360 | | |
| MRP2 | 23 | intron 15 + 169 | atgagctgaaagcaaaggtt T/C tcagccccttcccctgataa | 1361 | | |
| MRP2 | 24 | intron 15 + 949 | ttccaggtgacacatttagt A/G cctaatttgggaaatgttaa | 1362 | | |
| MRP2 | 25 | intron 15 + 984 | tgttaatctagtccaatccc A/C ttagtaagaaaggaggggtc | 1363 | | |
| MRP2 | 26 | intron 16 + 4059 | catcctgatgcacagttatt C/T aaatttaagctccatttgtt | 1364 | | |
| MRP2 | 27 | intron 19 + 10899 | atgtatggagtatttatgga G/A taaagtattccatgctgtat | 1365 | | |
| MRP2 | 28 | exon 22 + 51 | caagcaataggattgttttc G/A atattcttcatcatccttgc | 1366 | | |
| MRP2 | 29 | intron23 + 56 | tatactgaggatctttctga C/T agggaggaattattatgtcc | 1367 | | |
| MRP2 | 30 | intron 23 + 734 | tgagccaactactgtactag G/A cactggggcactcaatgaat | 1368 | | |
| MRP2 | 31 | intron 23 + 801 | atgggccagacccaactcac T/G gattttttagtgtatctgag | 1369 | | |
| MRP2 | 32 | intron 27 + 124 | gggtccctaaagtttccttt C/G ctctaactcaaaggacctaa | 1370 | | |
| MRP2 | 33 | exon 28 + 52 | cagattggcccagcaaaggc A/C agatccagtttaacaactac | 1371 | | |
| MRP2 | 34 | exon 28 + 84 | aacaactaccaagtgcggta C/T cgacctgagctggatctggt | 1372 | | |
| MRP2 | 35 | exon 28 + 129 | agagggatcacttgtgacat C/T ggtagcatggagaaggtagg | 1373 | | |
| MRP2 | 36 | intron 29 + 154 | ttccctaggatggacacgtc A/G tttccagaactttgaaatgt | 1374 | | |
| MRP2 | 37 | intron 30 + 91 | gtgttaggtgatgcctggca T/C agaattttcatccaggtctg | 1375 | | |
| MRP2 | 38 | intron 31 + 170 | gccaaaattttacatcacgc A/G aatgaaaacgaacaaggtta | 1376 | | |
| MRP2 | 39 | intron 26 + 154 | ctggctccatcttttaccca T/C ggacgtattccttactcttc | 1377 | | |
| MRP2 | 40 | 3′-flanking + 739 | gtgaatttttattataagct C/T gttctccttaaaactttatc | 1378 | | |
| MRP2 | 41 | intron 3 + 1145 | acatccttctcccctcagtc C/T tcggttagtggcagtattct | 1379 | | |
| MRP2 | 42 | intron23 + 432 | tggcagtagagcagggtgag G/A aggattattctgcagaggaa | 1380 | | |
| ABCB1 | 1 | 5′flanking − 196 | gctttggagccatagtcatg T/C actcaaaatttattttatct | 1381 | | |
| ABCB1 | 2 | 5′flanking − 18 | tactctttacctgtgaagag T/C agaacatgaagaaatctact | 1382 | | |
| ABCB1 | 3 | intron1 + 71660 | cttgctggaggaagggtgct A/C gaaaatataccaaatccaag | 1383 | | |
| ABCB1 | 4 | intron1 + 80091 | gaaataatattcaagttctg A/C aataatatcatgacctatag | 1384 | | |
| ABCB1 | 5 | intron1 + 103126 | gatgtgaatcagaattcatc T/C gtgtctcaagaaaaggtcat | 1385 | | |
| ABCB1 | 6 | intron1 + 103148 | tgtctcaagaaaaggtcatg C/T gataaattaagttctgctag | 1386 | | |
| ABCB1 | 7 | intron1 + 108428 | aattaatttatcatcatctg A/G tcaccatttcacacaactca | 1387 | | |
| ABCB1 | 8 | intron1 + 112042 | cataagttgaaatgtcccca A/G tgattcagctgatgcgcgtt | 1388 | | |
| ABCB1 | 9 | intron2 + 491 | gctctctggcttcgacgggg G/Δ actagaggttagtctcacct | 1389 | | |
| ABCB1 | 10 | intron4 + 36 | attaactattcaaaatactt C/T ggaaatttgacatctcctta | 1390 | | |
| ABCB1 | 11 | intron5 + 1596 | ttagctctcttactgcttca T/C agtggaagaatcaaatactt | 1391 | | |
| ABCB1 | 12 | intron8 + 1789 | aaacactctgaatattaaac C/T gctcctggaaccacagctca | 1392 | | |
| ABCB1 | 13 | intron14 + 24 | agttgtccttgccctttgcc T/C ttctagaggtgcaaaaaata | 1393 | | |
| ABCB1 | 14 | intron14 + 81 | tgcaggaagttaggaaacta C/T tataaatcggaagaagggaa | 1394 | | |
| ABCB1 | 15 | intron15 + 38 | caaaccaacctgatttataa A/G cataagaacattctactact | 1395 | | |
| ABCB1 | 16 | intron17 + 73 | gtttggtgggctagggctcc A/G gtaggagtgggaacaagaga | 1396 | | |
| ABCB1 | 17 | intron18 + 564 | caacagtaaagttacaatct G/A aaaggaatgctctctgttta | 1397 | | |
| ABCB1 | 18 | intron18 + 2062 | tttccctgaggaatggttat C/T ctctgtgttccttgagtcca | 1398 | | |
| ABCB1 | 19 | intron18 + 2293 | ccacatcaggttttccccag A/G caccttgggacagtttgaaa | 1399 | | |
| ABCB1 | 20 | intron20 + 557 | aaaaccctaaccattgacac G/A tgtgaatgttttcctgggga | 1400 | | |
| ABCB1 | 21 | intron21 + 24 | cgtgcctcctttctactggt G/A tttgtcttaattggccattt | 1401 | | |
| ABCB1 | 22 | intron21 + 2725 | ctgacctgtttttggctgac A/G ggttttagttcctcccctca | 1402 | | |
| ABCB1 | 23 | intron21 + 4725 | tcttggtattaaaagatcca A/G agagataggaatatgtaatt | 1403 | | |
| ABCB1 | 24 | intron22 + 8507 | tgcacttaggaaaaaaacaa T/C atggaaatgtgtaaaatata | 1404 | | |
| ABCB1 | 25 | intron22 + 8537 | tgtaaaatatactttttttt T/A aaaaaaaaggacacatttat | 1405 | | |
| ABCB1 | 26 | intron22 + 8565 | aggacacatttattcagcat T/C atgatcagactattacattt | 1406 | | |
| ABCB1 | 27 | intron22 + 8952 | caccttggtttcatggtttg G/A caaagtactggcctgtacca | 1407 | | |
| ABCB1 | 28 | intron22 + 9520 | caccaacaaatatctttttc A/G cagttgggtgggcatctggt | 1408 | | |
| ABCB1 | 29 | intron22 + 9836 | agactctgacttagacatga C/T ggcaggggaaagagagactt | 1409 | | |
| ABCB1 | 30 | intron24 + 377 | taaaatacagatgtgttgta C/A taagttctgcaagcctttgg | 1410 | | |
| ABCB1 | 31 | intron24 + 1493 | ggggaggtgtccaggcacga A/Δ catggagagctggacttgat | 1411 | | |
| ABCB1 | 32 | intron24 + 1495 | ggaggtgtccaggcacgaac A/T tggagagctggacttgatac | 1412 | | |
| ABCB1 | 33 | intron25 + 342 | tgcagccttgatcttctggg C/T tcaagcgatcctcctgcctc | 1413 | | |
| ABCB1 | 34 | intron26 + 134 | cttggataaagtctgagagc C/G taaatatggtctccaagtgg | 1414 | | |
| ABCB1 | 35 | intron26 + 1272 | gtccttcaattttgtggtga A/G cttaaaaacaggactctaaa | 1415 | | |
| ABCB1 | 36 | intron26 + 1394 | tattaagtggtgtgttaaag A/G ttgtgctataatgaattgta | 1416 | | |
| ABCB1 | 37 | intron26 + (1987-1988) | aagggctggaagagtgaaag (AAAG) gaggctatttgctcccagac | 1417 | | |
| ABCB1 | 37 | intron26 + (1987-1988) | aagggctggaagagtgaaag gaggctatttgctcccagac | 1418 | | |
| ABCB1 | 38 | intron27 + 59 | gcagcctctctggcctatag G/T ttgatttataaggggctggt | 1419 | | |
| ABCB1 | 39 | intron27 + 80 | ttgatttataaggggctggt T/C tcccagaagtgaagagaaat | 1420 | | |
| ABCB3 | 1 | intron3+ 8 | tctcctttggcaggtaggtg G/A tgggcagctgggtccatttg | 1421 | | |
| ABCB3 | 2 | intron4+ 104 | cttcacccgtatgccaggac C/T tggggatgcttttctcttgt | 1422 | | |
| ABCB3 | 3 | intron10+ 219 | gcagcagtggtgctccctcc A/G tgggcagccccgtcaggtcc | 1423 | | |
| ABCB3 | 4 | intron11+ (317-319) | atggtgcccaggtggatgtg GTG/Δ tccatctcattcctgtcttt | 1424 | | |
| ABCB3 | 5 | exon12 + 19 | agctgcaggactggaattcc T/C gtggggatcgcacagtgctg | 1425 | | |
| ABCB3 | 6 | exon12+ (356-357) | aggtggggtggggtggggtg GG/TGGTGGGGTGGA ggct | 1426 | | |
| | | | tctgtgtccaggaaa | | | |
| ABCB7 | 1 | intron1 + 220 | acggggcaggaggttctggg C/A agaggacacctggagcgctg | 1427 | | |
| ABCB7 | 2 | intron1 + 480 | agttaactcccttgctgaca G/A gcgtgcttcttgataggcca | 1428 | | |
| ABCB7 | 3 | intron1 + (512-513) | gataggccaaaaccgtaact AT/Δ ctttccaaaacatagaccgc | 1429 | | |
| ABCB7 | 4 | intron1 + 1690 | agttctccaataaggcagat G/A aagttaagataaaatttgta | 1430 | | |
| ABCB7 | 5 | intron1 + 5309 | aattaatatcatttattgct G/A tattgttgtcagtgttatct | 1431 | | |
| ABCB7 | 6 | intron1 − 11274 | tgcttcttttcaagccagcc A/G gctttaaaaaaaagttagct | 1432 | | |
| ABCB7 | 7 | intron1 − 11085 | caggttttcagggctcatgt A/G gacctgaagaaaaatgagag | 1433 | | |
| ABCB7 | 8 | intron1 − 10037 | attctactttctcaacttct T/C ttattacattatctcatcat | 1434 | | |
| ABCB7 | 9 | intron1 − 21 | ccactctgaaacttccccct G/A ctttttttccttgtcagcag | 1435 | | |
| ABCB7 | 10 | intron3 + (135-136) | ttctctaatgaaaaaaaaaa (A) catattaattgaccatagtt | 1436 | | |
| ABCB7 | 10 | intron3 + (135-136) | ttctctaatgaaaaaaaaaa catattaattgaccatagtt | 1437 | | |
| ABCB7 | 11 | intron3 + 333 | aaaacaatttgtgtgtgtgc G/A tgtgcttcaaggttaatgtt | 1438 | | |
| ABCB7 | 12 | intron12 + 524 | taaccactctgccctcagta G/T gaaacacagtgccgaaccca | 1439 | | |
| ABCB7 | 13 | intron13 + 1543 | atcctgtgaggtggggaagc G/A tatggctagcataaatataa | 1440 | | |
| ABCB7 | 14 | intron13 + 2400 | tgttaccttactgcctcatt C/G tcattcttcccacctgctat | 1441 | | |
| ABCB7 | 15 | intron15 + 2201 | ctccttcctaaccttagcaa G/C agtctggagatttacttatc | 1442 | | |
| ABCB8 | 1 | 5′flanking − 2272 | ggcttaggcctaagggctga T/C gttggggccagtacccctga | 1443 | | |
| ABCB8 | 2 | 5′flanking − 2070 | agctatgaaaacaagaccct G/A tccttctagaggtagcaaaa | 1444 | | |
| ABCB8 | 3 | intron1 + 25 | aaacggaaaaacctactcag A/C gcgggccattgaccgcccgg | 1445 | | |
| ABCB8 | 4 | exon2 + 308 | tgctggtcctgggggtagcc G/A tcgtggtgaggctttcccca | 1446 | | |
| ABCB8 | 5 | intron2 + 334 | cccccacttaaaacatttgt C/G ccctctgtctccccattcca | 1447 | | |
| ABCB8 | 6 | intron4 + 12 | cctgctccggtactgccagc C/T gcagggtgcagagttggggt | 1448 | | |
| ABCB8 | 7 | intron5 + 547 | agttcatagcattctcgctc G/A gccccctcaggcctgctgct | 1449 | | |
| ABCB8 | 8 | exon7 + 57 | ggcaatgtgcggactgtgcg A/T gccttcgccatggagcaacg | 1450 | | |
| ABCB8 | 9 | intron9 + 1231 | tttccgcagctgcatggaca C/T cctcgcgtgccccgtttctg | 1451 | | |
| ABCB8 | 10 | intron9 + 2164 | cctcttggaggtccttctag C/T gctgcctatgtggagattct | 1452 | | |
| ABCB8 | 11 | intron9 + 2645 | ttcctgcctggtgcctcccc C/Δ ggctgcctttagcaagtgct | 1453 | | |
| ABCB8 | 12 | intron9 + 2646 | tcctgcctggtgcctccccc G/A gctgcctttagcaagtgctg | 1454 | | |
| ABCB8 | 13 | intron9 + 3229 | cagggccgagcagggagtcc G/A tgggtcagctgggctccctt | 1455 | | |
| ABCB8 | 14 | intron12 + (113-114) | tcctccactgccaccagggg (GG) ccttctttcctgggacaatc | 1456 | | |
| ABCB8 | 14 | intron13 + (113-114) | tcctccactgccacaagggg ccttctttcctgggacaatc | 1457 | | |
| ABCB8 | 15 | intron13 + 128 | tgctctcgggagaccctggc C/T gtcttcacatgtcctcagct | 1458 | | |
| ABCB8 | 16 | intron13 + 305 | atccaggtctagagaagcct A/G tagtggaggtgctgagctgc | 1459 | | |
| ABCB8 | 17 | intron14 + 135 | acagttgtgtcagggaagac C/G agaaccacagccaaagggga | 1460 | | |
| ABCB8 | 18 | intron14 + 159 | accacagccaaaggggacag A/T gtcgttgtgtggggacaggg | 1461 | | |
| ABCB8 | 19 | intron15 + 747 | gttggagccttgggctctgt A/G agggggacagagggaatcat | 1462 | | |
| ABCB8 | 20 | 3′flanking + 333 | cctatcccctggctcacccc G/A cagtgcattgatggagcagc | 1463 | | |
| ABCB8 | 21 | 3′flanking + 1168 | ccctctttcaggggtgtgat G/A cagtgcattgatggagcagc | 1464 | | |
| ABCB8 | 22 | 3′flanking + (1719-1721) | tagaccgcaggagccgcgcc GTC/Δ ttcctaacctcgcctcggcc | 1465 | | |
| ABCB9 | 1 | intron1 + 69 | agggtgccaggccaggcacg G/C gttggggggcgtctgggcac | 1466 | | |
| ABCB9 | 2 | intron1 + 8873 | tgggcccagcacgtggggcc T/C ggaactacctcaaaggcttc | 1467 | | |
| ABCB9 | 3 | intron1 + 8940 | accagctcagcctgcccagc G/A tgcacacggcaccaagctgg | 1468 | | |
| ABCB9 | 4 | intron1 + 11410 | agatccaagggatccagagg T/C tggaatgtgaccctccgtgc | 1469 | | |
| ABCB9 | 5 | intron1 + 12863 | gggaagccagatgcccacaa G/A gctctgtgacttcacttcca | 1470 | | |
| ABCB9 | 6 | intron1 + 19731 | gccaagtgtcaagatcgagc G/A aggggagggcctgacgaggg | 1471 | | |
| ABCB9 | 7 | intron1 + 29649 | cagaatccagatgcccgtaa T/C gttgttaagaagcctgcaca | 1472 | | |
| ABCB9 | 8 | intron1 + 31793 | ggccaggcggggaggggtac C/T ggccagaccggtgggcaaaa | 1473 | | |
| ABCB9 | 9 | intron1 + 37537 | agagtcacagggttggggtg C/A ccccgggaaggtggcatcta | 1474 | | |
| ABCB9 | 10 | intron1 + 38293 | taccagccctgtgctttcag G/A gaccatgtgacctgtcaact | 1475 | | |
| ABCB9 | 11 | intron1 + 44661 | cccgaggtgcctggcttcac A/G gcaggattgccgtcctgcag | 1476 | | |
| ABCB9 | 12 | intron1 + 49576 | aaagtggccccgtggcttgt C/T ccctgaagccctaaagcacc | 1477 | | |
| ABCB9 | 13 | intron1 + 64669 | ccacagacaagccgggtagc C/A cacctcgcagctcaacacac | 1478 | | |
| ABCB9 | 14 | exon2 + 448 | cctggttttgggccctgttc G/A tgtggacgtacatttcactc | 1479 | | |
| ABCB9 | 15 | intron7 + 3364 | ggtaccaggagtcgggtatc A/G gtgggacaggaacgcgtgtc | 1480 | | |
| ABCB9 | 16 | intron11 + 113 | gggccccaggagctctccca G/T actatcagcctcctgggctg | 1481 | | |
| ABCB9 | 17 | exon12 + 370 | cccaggcctgcagcactgaa A/G gacgacctgccatgtcccat | 1482 | | |
| ABCB10 | 1 | 5′flanking − 424 | tcgcgtctgcgcgctccgcc C/T ggtctgccggcgtgagaaag | 1483 | | |
| ABCB10 | 2 | exon1 + 491 | acaaggggcggttgcgcccc G/T cagcggccggactcccggag | 1484 | | |
| ABCB10 | 3 | intron1 + 37 | ccacttccctccgccgggcc T/G ctccttctccacacgcgggg | 1485 | | |
| ABCB10 | 4 | intron1 + 217 | actcgtttgcagattttaca C/T ttgttttcttgttgacacac | 1486 | | |
| ABCB10 | 5 | intron1 + 405 | gcgtttatactttttttttt T/Δ aaccaaaaacacattatttg | 1487 | | |
| ABCB10 | 6 | exon3 + 185 | agggccggggcccaggcttc C/T gtaggcatcagtatgatggt | 1488 | | |
| ABCB10 | 7 | intron6 + 1269 | caaattcacaactgtgcctt C/G cacagaatgggttggaaaac | 1489 | | |
| ABCB10 | 8 | intron9 + 632 | ccccactccacttgggtgag G/A gcaggtggatggtgatgggt | 1490 | | |
| ABCB10 | 9 | intron10 + 2373 | tacctcagggcactcagaca G/C cctcaccaatcagaggctca | 1491 | | |
| ABCB10 | 10 | intron11 + 108 | tccttttcctgttttttgtt T/G tttttttttcttgggagtgg | 1492 | | |
| ABCB10 | 11 | intron11 + 2379 | cattggtttttagtgtattc T/A gtgttgtgcatccatcatca | 1493 | | |
| ABCB11 | 1 | 5′flanking − (2596-2595) | tgtggtttagagctttctct (TT) gagacatttttgctaaggtt | 1494 | | |
| ABCB11 | 1 | 5′flanking − (2596-2595) | tgtggtttagagctttctct gagacatttttgctaaggtt | 1495 | | |
| ABCB11 | 2 | 5′flanking − 1746 | agctgaagtgaattaagcac G/A atcaactcagtactcacact | 1496 | | |
| ABCB11 | 3 | 5′flanking − (326-314) | agggggacagtttaaaggta (T)9-12 gtcttgttatgtttttaagt | 1497 | | |
| ABCB11 | 4 | 5′flanking − 135 | agagggtttcccaagcacac T/C ctgtgtttggggttattgct | 1498 | | |
| ABCB11 | 5 | intron1 + 511 | aaatatagatgcaaaaaaaa A/Δ tgagctgtggatgcatgttt | 1499 | | |
| ABCB11 | 6 | intron1 + 581 | aatttcagtttttaggtcac C/T caagccagtgggagtcacat | 1500 | | |
| ABCB11 | 7 | intron1 + (1938-1951) | aaagacgttttaagggcttt (A)10-13 gaaagaaaagaaaactgtag | 1501 | | |
| ABCB11 | 8 | intron1 + 4517 | ggtttcccaacatctcatct G/A ataaaaaaaataatttgcca | 1502 | | |
| ABCB11 | 9 | intron1 + 5651 | aaagagaataggttagtgga T/C tagtattcctgtgcttaatg | 1503 | | |
| ABCB11 | 10 | intron1 + (12200-12201) | aagagatggtctctagcccc CT/Δ gtttgatttggggcacttac | 1504 | | |
| ABCB11 | 11 | intron1 + 13023 | gtttggctactttgattaaa G/A aagaaagaagagataataat | 1505 | | |
| ABCB11 | 12 | intron2 + 739 | cctgcatctattctgaccta C/T actggggaaaacagtatgtg | 1506 | | |
| ABCB11 | 13 | intron2 + (921-922) | tattttgtagttcaaaaagt (CAGATCTTCTTCAGCT | 1507 | | |
| | | | AATTTAGAAATGT) tgctgccatttgatattca | | | |
| ABCB11 | 13 | intron2 + (921-922) | tattttgtagttcaaaaagt tgctg | 1508 | | |
| | | | tccatttgatattca | | | |
| ABCB11 | 14 | intron3 + 644 | agccacacgtttcttattgc G/A tgggagtttaaaaaaatggg | 1509 | | |
| ABCB11 | 15 | intron3 + 2231 | agtgaacctgagattgagct A/G tactgaaatctctagaagag | 1510 | | |
| ABCB11 | 16 | intron3 + 2406 | aaagggtggtctttaaatcc T/C tatgtttttctcatcaggtt | 1511 | | |
| ABCB11 | 17 | exon4 + 10 | tttctcatcaggttacaaga T/C gagaagaaaggtgatggcgt | 1512 | | |
| ABCB11 | 18 | intron4 + 434 | acaatttatagtatttctca A/G tgccccacacagtttatcta | 1513 | | |
| ABCB11 | 19 | intron4 + 518 | gtagatgagtagctaaaaac G/T aaagtcagctcctgaaataa | 1514 | | |
| ABCB11 | 20 | exon5 + 120 | ggcacaatgacagatgtttt T/C attgactacgacgttgagtt | 1515 | | |
| ABCB11 | 21 | intron5 + 320 | gggaggtgacccatgaattt T/C acttgagtatcatctccaag | 1516 | | |
| ABCB11 | 22 | intron5 + 16076 | agaagaggtaacagtaagcc T/G cctgatttacagcacacatc | 1517 | | |
| ABCB11 | 23 | intron6 + 303 | atttgcaggtgtgtttgtag G/C gggcagttgagtagcttgaa | 1518 | | |
| ABCB11 | 24 | intron7 + 1141 | aaaggattcagcaggcatga A/G gaaagaaaagctttgcaaga | 1519 | | |
| ABCB11 | 25 | intron8 + 2463 | ccattggctaatagcaatga A/C ctatgacatggtctaactta | 1520 | | |
| ABCB11 | 26 | intron8 + 2677 | tcaatgatgttacagtgaga A/C tctaatattgtattaaaccc | 1521 | | |
| ABCB11 | 27 | intron8 + 2699 | ctaatattgtattaaaccca T/A gccacatgttaaatgaatct | 1522 | | |
| ABCB11 | 28 | intron9 + 24 | gtgtccaagtttacggacta T/C gagctgaaggcctatgccaa | 1523 | | |
| ABCB11 | 29 | intron10 + 108 | caccttggtctgtggcctcc A/G gaggaagtacttgttcaaga | 1524 | | |
| ABCB11 | 30 | intron10 + 2475 | taatcattccaaaccacgga C/A tttatttcattaagaacatg | 1525 | | |
| ABCB11 | 31 | intron10 + 2478 | tcattccaaaccacggactt T/A atttcattaagaacatgata | 1526 | | |
| ABCB11 | 32 | intron10 + 2711 | tttacagattggaaaagcca C/T tgaagtcttgcaggtccaga | 1527 | | |
| ABCB11 | 33 | intron10 + 3539 | agtgactgtaattagtatca C/G ttgtgcacagagaaaaaatg | 1528 | | |
| ABCB11 | 34 | intron10 + 3623 | tgcagaaggttgttctttca T/C gaccttcctgagtttcagaa | 1529 | | |
| ABCB11 | 35 | intron10 + 3661 | gaattcattaataaaaataa A/T cacataatggagcgtgacat | 1530 | | |
| ABCB11 | 36 | intron10 + 5100 | gggccactctttggcttggc A/G atagactgtggccaatgaaa | 1531 | | |
| ABCB11 | 37 | intron10 + 5292 | gctatttggtaggaacatct G/A ggcatgatcaggtagccttc | 1532 | | |
| ABCB11 | 38 | intron10 + 5912 | gagtaatattcagtaaaaaa A/Δ taaagtggtattttaaatca | 1533 | | |
| ABCB11 | 39 | intron12 + 116 | tgtttccagtaatagggaat G/A gaggtgtctttctctgaaag | 1534 | | |
| ABCB11 | 40 | intron12 + 326 | gataaatgacaaggcaatta G/C aacaatcaggaagcacaggt | 1535 | | |
| ABCB11 | 41 | intron12 + 335 | caaggcaattacaacaatca A/G gaagcacaggttcttcccaa | 1536 | | |
| ABCB11 | 42 | intron12 + 2572 | cctcatccttgccaatgttt C/T cttttactggtttttgatgg | 1537 | | |
| ABCB11 | 43 | exon13 + 23 | tctaaatgacctcaacatgg T/C cattaaaccaggggaaatga | 1538 | | |
| ABCB11 | 44 | intron13 + 70 | atggcagtatattgatcaaa C/T agaaaggtgtagcatacatt | 1539 | | |
| ABCB11 | 45 | intron13 + (1578-1579) | ttattggcctctattttttc (C) tgcccattggtcaagtatga | 1540 | | |
| ABCB11 | 45 | intron13 + (1578-1579) | ttattggcctctattttttc tgcccattggtcaagtatga | 1541 | | |
| ABCB11 | 46 | intron14 + 32 | catacattcctgggagaaac C/T aagaggtcatagaaggaaaa | 1542 | | |
| ABCB11 | 47 | intron14 + 80 | cacaattatacacatttctt C/T tcgtatgattcccaagtcat | 1543 | | |
| ABCB11 | 48 | intron14 + 439 | tattgtgtcaaaaacaattc A/G ttgtatatctccattctaag | 1544 | | |
| ABCB11 | 49 | intron14 + (1262-1263) | cagcctttgcattatatttt (T) gctgtgttgtctaacaggag | 1545 | | |
| ABCB11 | 49 | intron14 + (1262-1263) | cagcctttgcattatatttt gctgtgttgtctaacaggag | 1546 | | |
| ABCB11 | 50 | intron14 + 1283 | gctgtgttgtctaacaggag A/C aaagagacacggatttgctc | 1547 | | |
| ABCB11 | 51 | intron14 + 1339 | tgagatagatatttaggacc G/A tgaccaatttttattttggt | 1548 | | |
| ABCB11 | 52 | intron14 + 1359 | gtgaccaatttttattttgg T/C tgaaaaatcttatttgaagt | 1549 | | |
| ABCB11 | 53 | intron14 + 1480 | tattgattagacaataaccc G/A tctggggaagggatatttct | 1550 | | |
| ABCB11 | 54 | intron15 + 370 | ccttttctaatgtctgcaca G/A cctatttaagaatattccca | 1551 | | |
| ABCB11 | 55 | intron16 + (550-559) | aaagtttagtgtttctatca (T)9-12 gctacttctgatggacttct | 1552 | | |
| ABCB11 | 56 | intron17 + 188 | tttctctccccaattcatgg T/G tttttggttagcttctcatc | 1553 | | |
| ABCB11 | 57 | intron17 + 194 | tccccaattcatgggttttt T/G gttagcttctcatcttcttg | 1554 | | |
| ABCB11 | 58 | intron17 + (197-198) | caattcatgggttttgggtt (T) agcttctcatcttcttgggg | 1555 | | |
| ABCB11 | 58 | intron17 + (197-198) | caattcatgggtttttggtt agcttctcatcttcttgggg | 1556 | | |
| ABCB11 | 59 | intron17 + (289-296) | ttagaaaggggacttctttt (A)7g(A)4 tctgtgtttagtgttcctct | 1557 | | |
| ABCB11 | 59 | intron17 + (289-296) | ttagaaaggggacttctttt (A)12 tctgtgtttagtgttcctct | 1558 | | |
| ABCB11 | 59 | intron17 + (289-296) | ttagaaaggggacttctttt (A)10 tctgtgtttagtgttcctct | 1559 | | |
| ABCB11 | 60 | intron17 + 1070 | tcagacttgggttttcctat C/T tttcttcttgagaacaagtt | 1560 | | |
| ABCB11 | 61 | intron17 + 1651 | tgttaaaatatctcattgta T/C atgctgacggatttttcttg | 1561 | | |
| ABCB11 | 62 | intron17 + 2226 | ccttaagtctcctcctatca T/A gcaccttgttctcaccagct | 1562 | | |
| ABCB11 | 63 | intron17 + 2979 | ctctctcttcctttctcagc T/Δ ctactatttcactgttggct | 1563 | | |
| ABCB11 | 64 | intron17 + 3288 | aatccccatatcctacctta T/G ccatctcatccatgaatctt | 1564 | | |
| ABCB11 | 65 | intron17 + 3289 | atccccatatcctaccttag C/T catctcatccatgaatcttg | 1565 | | |
| ABCB11 | 66 | intron18 + 97 | aatatgagttttctaggtat A/G tatctagcagtgtttcaagt | 1566 | | |
| ABCB11 | 67 | intron18 + 98 | atatgagttttctaggtata T/C atctagcagtgtttcaagtc | 1567 | | |
| ABCB11 | 68 | intron18 + 892 | ctctgaaagttagtgataca C/T cttatttgtgtttgaatcaa | 1568 | | |
| ABCB11 | 69 | intron18 + 2681 | atgtatgagatcaagtcagg A/G tcaaatattagacacccata | 1569 | | |
| ABCB11 | 70 | intron18 + 3780 | ggaccatcctgtggggcaat C/G gttccagaaaatgctggtat | 1570 | | |
| ABCB11 | 71 | intron18 + 5741 | ctcaccggtataaatacaac C/T gtagcaaaggttttcttttt | 1571 | | |
| ABCB11 | 72 | intron18 + (5882-5883) | tgcgtattccctcagttcag (C) tttttattcaagccacagca | 1572 | | |
| ABCB11 | 72 | intron19 + (5882-5883) | tgcgtattccctcagttcag tttttattcaagccacagca | 1573 | | |
| ABCB11 | 73 | intron21 + 10022 | tggctaagttaaaaaaaaaa A/Δ gagattcaactataattgct | 1574 | | |
| ABCB11 | 74 | intron22 + 322 | caagattcaatactgccccc C/Δ agggggtgggtgaacagggc | 1575 | | |
| ABCB11 | 75 | intron22 + 257 | ctgttcaatttcctctcgca T/C agtgattcattccacattcc | 1576 | | |
| ABCB11 | 76 | intron22 + 552 | taattaatatcttgtccttg G/C ggggtaaatgagggatggta | 1577 | | |
| ABCB11 | 77 | intron22 + 569 | ttggggggtaaatgagggat G/A gtagcataaacacttctcaa | 1578 | | |
| ABCB11 | 78 | 3′flanking + 243 | aaacaccacagaatgacata G/A aactacaggcggcaggaatc | 1579 | | |
| CYP4B1 | 1 | 5′flanking − 333 | gaaacattcacagtgcttgt A/T tgagaagacagtggttatta | 1580 | | |
| CYP4B1 | 2 | 5′flanking − 18 | gagcagctgaaggcaggtca G/T atgaaggctaggtggctgga | 1581 | | |
| CYP4B1 | 3 | intron1 + 341 | tccaaaacctctggatagta C/T atagaagtaggcaatccatt | 1582 | | |
| CYP4B1 | 4 | intron1 + 542 | cctatgggtggctcaggagc C/T gtgacaccttcccaggttca | 1583 | | |
| CYP4B1 | 5 | intron1 + 2856 | gaggactttgcacatagtag G/A tgctcagctatattgttggc | 1584 | | |
| CYP4B1 | 6 | intron1 + 6086 | tttggaatctaaagactggg G/T cacgatgctagttgtgtgac | 1585 | | |
| CYP4B1 | 7 | intron1 + 6598 | ttttggggtgtggggagagg G/A cccatagtagggagacagct | 1586 | | |
| CYP4B1 | 8 | intron1 + 6660 | acctaagggtgtccatcctg A/G aggagagcagtcctaggggg | 1587 | | |
| CYP4B1 | 9 | intron1 + 7242 | ccctggtctcccttaactca T/C gctggactgttccctttggt | 1588 | | |
| CYP4B1 | 10 | intron2 + 107 | gcctgtgtactaagtctgcg C/G agctgaggttcccaccctac | 1589 | | |
| CYP4B1 | 11 | intron3 + 361 | atggtgtggtggtaggacca C/T ggctggtcaccagaggctgt | 1590 | | |
| CYP4B1 | 12 | intron4 − 492 | aaaggctttcacatctaaaa C/A gtgtctcctcattttctgtc | 1591 | | |
| CYP4B1 | 13 | intron4 − 315 | ggattacttacatatacacc A/G tgcgggggagctcaccacct | 1592 | | |
| CYP4B1 | 14 | intron4 − 157 | ctacccaccctatcctgata T/C tccagcaggatggagggcag | 1593 | | |
| CYP4B1 | 15 | exon5 + 22 | acaagtgggaagagaaagct C/T gggagggtaagtcctttgac | 1594 | | |
| CYP4B1 | 16 | intron5 + 125 | cccagggagccttagcttgc G/A gggagacaggacctgctcat | 1595 | | |
| CYP4B1 | 17 | intron5 + (287-289) | tgtctaagccaatccctcct CCT/Δ accctctgcttagcagggac | 1596 | | |
| CYP4B1 | 18 | intron6 + 54 | gcctgggttcctcctcctgg C/T ccctctatgccccctcccat | 1597 | | |
| CYP4B1 | 19 | intron7 + (99-100) | agctcttaagcatttccccc (TC) tttcctcagcaaatataacc | 1598 | | |
| CYP4B1 | 19 | intron7 + (99-100) | agctcttaagcatttccccc tttcctcagcaaatataacc | 1599 | | |
| CYP4B1 | 20 | exon8 + 114 | tcctggtttctctactgcat G/A gccctgtaccctgagcacca | 1600 | | |
| CYP4B1 | 21 | exon8 + 139 | tgtaccctgagcaccagcat C/T gttgtagagaggaggtccgc | 1601 | | |
| CYP4B1 | 22 | intron8 + 247 | agaaagttgtcaacaagagg C/T tgatattttgtgtgctaact | 1602 | | |
| CYP4B1 | 23 | intron8 + 366 | tgtgggggtgaacagagctg A/G gacagctgggagagccagtt | 1603 | | |
| CYP4B1 | 24 | intron8 + 650 | cctttgcttgtggtcagaca C/A cctgcctttctctctgggct | 1604 | | |
| CYP4B1 | 25 | intron8 + 844 | tcatatgtgagaatcccccc C/A ccacggggtatccagacaca | 1605 | | |
| CYP4B1 | 26 | intron8 + 1767 | tcccattccaagaatgttct G/T gttgtgttgctggcagggat | 1606 | | |
| CYP4B1 | 27 | exon9 + 53 | tgtgcatcaaggagagcttc C/T gcctctacccacctgtgccc | 1607 | | |
| CYP4B1 | 28 | intron9 + 652 | agtcggatgtggtcatgaac G/T ctctgtcactggcagtggtc | 1608 | | |
| CYP4B1 | 29 | intron9 + 774 | cctggtcaccaacctctgtt C/T tgcccacaggaagcctgatc | 1609 | | |
| CYP4B1 | 30 | intron10 + 33 | tgggctgggagatcagacag G/T gtgggggactgggagggtca | 1610 | | |
| CYP4B1 | 31 | exon12 + 224 | ccagatggctcaggctgtga C/A ctccctgggcaccaccctcc | 1611 | | |
| CYP4B1 | 32 | exon12 + 270 | ctgggtgtggaggagttggg G/A ccccctgccttcaggaggct | 1612 | | |
| CYP4B1 | 33 | 3′flanking + 129 | tctgtgtctcacagtcacgt G/A gtgctccaggcattcagggt | 1613 | | |
| CYP27A1 | 1 | intron1 + 295 | aggagggagctgtcttggga A/G gagagtggcagaggcaaatg | 1614 | | |
| CYP27A1 | 2 | intron1 + 17503 | cagtgcataaagcctctgat C/T ctccttagagaaggagggac | 1615 | | |
| CYP4F2 | 1 | intron1 + (145-146) | ccaagcccctggcaacctca CA/Δ gtgattcaggctgggccttt | 1616 | | |
| CYP4F2 | 2 | intron1 + 193 | tttaatcagtctctctctct C/T tttcccattctaagtgctta | 1617 | | |
| CYP4F2 | 3 | intron1 + 324 | ccctgctctacctccggcac T/C gcccgtccctgcctctccac | 1618 | | |
| CYP4F2 | 4 | intron1 + 367 | tccctggaggtccctgggcc G/C ttctctgggcctcaggatct | 1619 | | |
| CYP4F2 | 5 | intron1 + 402 | ggatctcaccgtccatcccg T/C gcccgtccctgcctctccac | 1620 | | |
| CYP4F2 | 6 | exon2 + 35 | gcctgtcctggctgggcctc T/G ggccagtggcagcatcccct | 1621 | | |
| CYP4F2 | 7 | exon2 + 166 | cggtgtttcccacaaccccc A/G agacggaactggttttgggg | 1622 | | |
| CYP4F2 | 8 | intron2 + 125 | ggcagagaagcagaggaggc A/G tcttactcattcctctgctt | 1623 | | |
| CYP4F2 | 9 | intron2 + 440 | gggccgtctcccacttccac T/C acacccgaaggcacctttct | 1624 | | |
| CYP4F2 | 10 | exon3 + 48 | gttctgactcagctggtggc C/T acctacccccagggctttaa | 1625 | | |
| CYP4F2 | 11 | intron3 + 701 | agactccaccccagcttggg T/A ccctttccttgacccctgtg | 1626 | | |
| CYP4F2 | 12 | intron3 + 742 | cttcccatcgttggacgggc G/A aggctgagcagggggaatgg | 1627 | | |
| CYP4F2 | 13 | intron3 + 1020 | gctttagctttctccatgtc G/A cttttcctatcaaggtggcc | 1628 | | |
| CYP4F2 | 14 | intron3 + 1039 | cgcttttcctatcaaggtgg C/A cttttcctcatgatgtcaac | 1629 | | |
| CYP4F2 | 15 | intron3 + 1040 | gcttttcctatcaaggtggc C/G ttttcctcatgatgtcaacg | 1630 | | |
| CYP4F2 | 16 | intron3 + 1920 | ccacctgtctaacctctgtt G/C ctgtttgctcatgtctgggg | 1631 | | |
| CYP4F2 | 17 | intron3 + 1945 | ttgctcatgtctggggcgtg T/ ctctacaaatggctgttatat | 1632 | | |
| CYP4F2 | 18 | intron3 + 2621 | agcattctgtagaatgctga G/A ctgtgctcaggggttgcgga | 1633 | | |
| CYP4F2 | 19 | intron3 + 2665 | tgttggatcgtgtaggaggc A/G tgtcaaggcatgctggaacc | 1634 | | |
| CYP4F2 | 20 | intron6 + 194 | gggtttgaactggtgggtgt G/T gtcagagctctgtaggggac | 1635 | | |
| CYP4F2 | 21 | intron7 + 67 | tgtgaaatgtcagatgaaag G/A atttgaacttgattaagagg | 1636 | | |
| CYP4F2 | 22 | intron7 + 2811 | ttccaagggaaattgccatt T/G aattctcctgtaactcaggt | 1637 | | |
| CYP4F2 | 23 | intron7 + (3096-3097) | ggggtgggggttgggggggg (G) ttactgccttctctccagga | 1638 | | |
| CYP4F2 | 23 | intron7 + (3096-3097) | ggggtgggggttgggggggg ttactgccttctctccagga | 1639 | | |
| CYP4F2 | 24 | intron8 + 145 | ggtgctgtctaccttcgggt G/A ctgaagcagcccagagaccc | 1640 | | |
| CYP4F2 | 25 | exon9 + 44 | ctctcctgggtcctgtacca C/T cttgcaaagcacccagaata | 1641 | | |
| CYP4F2 | 26 | exon11 + 48 | gaacccatcacaacctagct G/A tgtggccggaccctgaggtg | 1642 | | |
| CYP4F2 | 27 | intron12 + 108 | tggtccaagttccagctctc C/T ttccctcacctcctctggag | 1643 | | |
| CYP4F2 | 28 | intron12 + 285 | gcatggggatccaggcacgg A/T tacccccttctctattcctc | 1644 | | |
| CYP4F2 | 29 | exon13 + 238 | aagtgaagcctagaattacc C/A taagaccctgttccacagtc | 1645 | | |
| CYP4F2 | 30 | exon13 + 342 | tgtgcgtgaatgttcatggc G/A gccctattcacagtagccaa | 1646 | | |
| CYP4F2 | 31 | exon13 + 563 | tagtgtactgtccttttata T/C gaaatttccagaacaggcca | 1647 | | |
| CYP4F2 | 32 | exon13 + 707 | aaatgttccggacctagata G/C tgacgaaggtagcacgacac | 1648 | | |
| CYP4F3 | 1 | intron2 + 258 | cattaatgcacctctgcggg G/T ctcttgggcagggggttggg | 1649 | | |
| CYP4F3 | 2 | intron2 + 916 | ttagggacatgtcctgagtc C/T acactgctccccacaaacct | 1650 | | |
| CYP4F3 | 3 | intron2 + 3417 | atccaggtctcacacagtgt C/T acttcctctcttggctttag | 1651 | | |
| CYP4F3 | 4 | intron2 + 4090 | gagagcatgaattgggtcct G/A tgtctttctctccagattca | 1652 | | |
| CYP4F3 | 5 | intron3 + 89 | tgtgctgcctccagcgggtc G/A cgtgcccatgtgcagacagg | 1653 | | |
| CYP4F3 | 6 | intron3 + 243 | tcaagtctgctgtacggcta C/T gtcttgtcacctgtatattt | 1654 | | |
| CYP4F3 | 7 | intron3 + 502 | aggtctgggacccagggtcc G/C taagtgaactgtctgagaca | 1655 | | |
| CYP4F3 | 8 | intron3 + 755 | ttttgtggccatgtcaggac A/T tgtgaacacatgtcagtgtc | 1656 | | |
| CYP4F3 | 9 | intron3 + 855 | gggacagacagggtgtccta G/A gtccttgtgaaggcattctg | 1657 | | |
| CYP4F3 | 10 | intron3 + 970 | cctgacatagctcctacgtg C/T catgttaggcagtgtcattg | 1658 | | |
| CYP4F3 | 11 | intron6 + 122 | gaggagttgttatacctgat C/T gttgaaggactggtatgaat | 1659 | | |
| CYP4F3 | 12 | exon7 + 159 | ggtgcacgacttcacagatg C/A cgtcatccaggagcggcgcc | 1660 | | |
| CYP4F3 | 13 | intron7 + 2107 | caggttgccagtgatttttt T/Δ ctcagaaagtttcatcaag | 1661 | | |
| CYP4F3 | 14 | intron7 + 2255 | gaccaagaagggtctaggag T/A gcaagatgggcttgggtttc | 1662 | | |
| CYP4F3 | 15 | intron8 + 132 | cctcaatgcaaggttgctgt A/C caccctcgggtgctgaagca | 1663 | | |
| CYP4F3 | 16 | exon9 + 59 | taccaccttgcaaagcaccc G/A gaataccaggagcgctgtcg | 1664 | | |
| CYP4F3 | 17 | intron9 + 13 | attgaatggtgagtgcaggt G/A ctggtgccctgttcctgagc | 1665 | | |
| CYP4F3 | 18 | intron9 + 36 | ggtgccctgttcctgagcct G/C tctcattggctctgttcccc | 1666 | | |
| CYP4F3 | 19 | intron9 + 167 | acccatcctgactgtctggg C/G aaaggttataggcccttagg | 1667 | | |
| CYP4F3 | 20 | intron9 + 369 | tccctaattcctacccttcc G/A tccagtccagggatttataa | 1668 | | |
| CYP4F3 | 21 | intron9 + 458 | tcattcatccatccagtcct T/C gttcagcaaatactctcata | 1669 | | |
| CYP4F3 | 22 | intron10 + 46 | ctcctgggtaggaagagggg A/C ccctcaggcagggagcattg | 1670 | | |
| CYP4F3 | 23 | intron10 + 63 | gggcccctcaggcagggagc C/A ttgtcctgactgcccccttc | 1671 | | |
| CYP4F3 | 24 | intron11 + 14 | ccctgaggtgcgggcccccc C/G tctctgtttttgtccattcc | 1672 | | |
| CYP4F3 | 25 | intron11 + 84 | gatcaggagaatccaacatc G/A cctccctccaagacacacac | 1673 | | |
| CYP4F3 | 26 | intron11 + 113 | caagacacacaccactgtct T/C tccaaggctggcggactggg | 1674 | | |
| CYP4F3 | 27 | intron11 + 164 | cggcaacccttcttggtctc T/G cctccaggtctatgacccct | 1675 | | |
| CYP4F3 | 28 | intron11 + 165 | ggcaacccttcttggtctcg T/C ctccaggtctatgacccctt | 1676 | | |
| CYP4F3 | 29 | intron12 + 156 | gaaaaggcccacagagtagg G/A ttgggttggtcctagaagga | 1677 | | |
| CYP4F3 | 30 | intron12 + 253 | gagctcggctaggctcgcag G/T atatgcaagcccacatgggg | 1678 | | |
| CYP4F3 | 31 | intron12 + 346 | tgggtgtcccaggccaggtt G/A ttgggttggtcctagaagga | 1679 | | |
| CYP4F8 | 1 | 5′flanking − 61 | accatgtttacccatcattg G/T tcctggagctccccagcccc | 1680 | | |
| CYP4F8 | 2 | exon1 + 67 | gtggcagcatccccgtggct G/T ctcctgctggtggtcggggc | 1681 | | |
| CYP4F8 | 3 | intron1 + 707 | tacgcagcaggtattcacca T/G tatttccacattatccactg | 1682 | | |
| CYP4F8 | 4 | intron1 + 857 | acaccccctaccctcacatc G/A tgacacagctgggccagaag | 1683 | | |
| CYP4F8 | 5 | intron1 + 907 | tgccatctccaccctccccc G/A tgcaggggcatcttctttat | 1684 | | |
| CYP4F8 | 6 | intron1 + 668 | tgtggcacttccaccatatg T/C tcattgccctcttgctccag | 1685 | | |
| CYP4F8 | 7 | intron2 + 818 | gccacagagaccatggctca G/A gccccaaaatgctgagtgac | 1686 | | |
| CYP4F8 | 8 | intron2 + 1079 | tatgcttgggtgttgcagaa C/T atgttggaccatgtaggagc | 1687 | | |
| CYP4F8 | 9 | intron2 + 1194 | ccggtcccctttatgccccc C/A accctcctttcttcttctgc | 1688 | | |
| CYP4F8 | 10 | intron5 + 45 | aacatgggatggagtggggg G/T gtgggtgtggggagagcaaa | 1689 | | |
| CYP4F8 | 11 | exon8 + (19-20) | ggccatgacaccacggccag (GCCAG) tggcctctcctgggtcttgt | 1690 | | |
| CYP4F8 | 11 | exon8 + (19-20) | ggccatgacaccacggccag tggcctctcctgggtcttgt | 1691 | | |
| CYP4F8 | 12 | intron8 + 222 | tttatttccccactaacttg C/G tatgcaagcttagtaaaatc | 1692 | | |
| CYP4F8 | 13 | intron8 + 334 | cttggagaattaacggcaaa A/T accgcaatgacttttggacc | 1693 | | |
| CYP4F8 | 14 | intron8 + 1999 | ttctaagtacatttattctc T/C tgcttttagctatgatctag | 1694 | | |
| CYP4F8 | 15 | intron8 + 4184 | caggagggccgtgtatgctc C/T ctggataattgttgggttgt | 1695 | | |
| CYP4F8 | 16 | exon9 + 119 | acgtggtgctcccagacagc C/T gagtcatccccaaaggtgcc | 1696 | | |
| CYP4F8 | 17 | intron11 + 282 | gggttgggggttccgggcct G/C gttcctggcgcagtggggcc | 1697 | | |
| CYP4F8 | 18 | intron11 + 340 | tgcagtcagaccttccacct C/T ggcccccaggaactgcatcg | 1698 | | |
| CYP4F8 | 19 | 3′flanking + 35 | atcacctacctttgcaccaa T/C taccttttcagatttccggt | 1699 | | |
| CYP4F8 | 20 | 3′fanking +3083 | ctgtgttggcccctgtgcct G/C agtcccgcggatggccagta | 1700 | | |
| CYP4F8 | 21 | 3′flanking + 90 | ggcccctgtgcctcagtccc A/G cggatggccagtagggggcg | 1701 | | |
| ALDH1 | 1 | intron1 + 564 | cattatttcttcagccaagt T/C tgttgccattggagcagatg | 1702 | | |
| ALDH1 | 2 | intron1 + 710 | gttctgagagtaactctgaa C/T tttgcctgtttcacactgct | 1703 | | |
| ALDH1 | 3 | intron1 − 3868 | ccctttttatatccagaata C/G agcctaaacttctttctctg | 1704 | | |
| ALDH1 | 4 | intron2 + 2933 | taagtatgctatactatatt T/C gatagatatactatactata | 1705 | | |
| ALDH1 | 5 | intron2 − 1646 | caatgtgattaactgaatgc C/T gcaaatatgcactgtatatg | 1706 | | |
| ALDH1 | 6 | exon3 + 54 | caggcttttcagattggatc C/T ccgtggcgtactatggatgc | 1707 | | |
| ALDH1 | 7 | intron3 + 157 | taggccccttaacattgaac T/G attctcaaatagtaatctgc | 1708 | | |
| ALDH1 | 8 | intron3 + 339 | tgagtctcctagaatgatat G/A ttaggtttattcaagcattt | 1709 | | |
| ALDH1 | 9 | intron3 + 655 | agcagttagatgagtcagag C/A ataatatagttgggggaggg | 1710 | | |
| ALDH1 | 10 | intron3 + 735 | gaagccaatttaacataaac C/A aataccaagatcaggtttca | 1711 | | |
| ALDH1 | 11 | intron3 + 863 | gcaagtatggttaatcaaag G/A accatttattactcaaatct | 1712 | | |
| ALDH1 | 12 | intron3 + 1757 | agatgacaagatttcttcta T/A ttcaaaaattccctagcaca | 1713 | | |
| ALDH1 | 13 | intron5 + 90 | ttctctaaaacagatggatg C/A ttatgtatttgttaaatgtg | 1714 | | |
| ALDH1 | 14 | intron6 + 213 | caggaagccaaacacaaagg T/C ttggtgtcaaacagtcaact | 1715 | | |
| ALDH1 | 15 | intron6 + 1323 | ttttgaattaaattcttata C/T tgtaacttttaaacttttta | 1716 | | |
| ALDH1 | 16 | intron7 + 638 | gcaaaagacagtggtggaag C/A atactgtaccatgcaaaaaa | 1717 | | |
| ALDH1 | 17 | intron9 + (1462-1463) | aatggcattctatgtttttt (T) gttgtgattatttatctatc | 1718 | | |
| ALDH1 | 17 | intron9 + (1462-1463) | aatggcattctatgtttttt gttgtgattatttatctatc | 1719 | | |
| ALDH1 | 18 | intron9 + 1757 | tgatctagaatttagtttct A/G taaatgaatagaatccagtg | 1720 | | |
| ALDH1 | 19 | intron12 − 1383 | aatcccacttattactctcc T/G gagagcttcaagtgcctata | 1721 | | |
| ALDH1 | 20 | 3′flanking + 40 | ttttaagtacaagttttggt T/C acagtgatttcttcttgtca | 1722 | | |
| ALDH2 | 1 | intron3 + 1766 | aaattggtggctcatcctgc C/Δ tggccgcccttcctcctcctc | 1723 | | |
| ALDH2 | 2 | intron8 + 52 | gaaggtagccctggccacct G/C tgttgtggctccagccgatc | 1724 | | |
| ALDH2 | 3 | intron8 + 69 | cctgtgttgtggctccagcc G/A atcctgtcgcccccccagtg | 1725 | | |
| ALDH2 | 4 | intron9 + 5197 | gctttcttatgaccttggtc C/A atttcccagttgtcttgttg | 1726 | | |
| ALDH2 | 5 | intron11 + 114 | gagctgggctcagtttctcc T/C gggtcagggtgtgatgtcga | 1727 | | |
| ALDH2 | 6 | 3′flanking + 411 | ggatatgatttctgcccctc T/C tctgctgtgggtaaacagct | 1728 | | |
| ALDH2 | 7 | 3′flanking + (432-433) | tctgctgtgggtaaacagct TC/Δ tgtttcatgcatttactttt | 1729 | | |
| ALDH2 | 8 | 3′flanking + 488 | ccaataagaatgtgcttgaa G/T gtttcatgcatttaatttgt | 1730 | | |
| ALDH7 | 1 | 5′flanking − 1455 | ctgcctgtccacacccacag C/T agcttgcacatcatccccac | 1731 | | |
| ALDH7 | 2 | intron1 + 464 | catgaatgactctgggaaag A/G atcattcttagcaatggact | 1732 | | |
| ALDH7 | 3 | intron1 + 2269 | aaatggaatccaaacagcaa G/C agacctcccctcaccggtca | 1733 | | |
| ALDH7 | 4 | intron2 + 1349 | actgagcttctgccaccggc C/T gcctgccggccttcatgaga | 1734 | | |
| ALDH7 | 5 | intron2 + 1820 | tccgtgtggaaggcaccttc C/G cccagcctcagtggctagga | 1735 | | |
| ALDH7 | 6 | intron2 + 2046 | aacctcaggcgctgcctcag C/G cagggagccagcctggcccc | 1736 | | |
| ALDH7 | 7 | intron2 + 2939 | aagcacgcactgaacatgga G/A tgagtgagtgaacgaatgaa | 1737 | | |
| ALDH7 | 8 | intron3 + 7 | tgcccaagaacctggtgagc C/T ggccgggctgaggcgggcag | 1738 | | |
| ALDH7 | 9 | intron4 + 36 | gccccttccggtcacccttc T/C ccgctcgaggcctcagggcc | 1739 | | |
| ALDH7 | 10 | intron6 + (116-117) | attctcctctctctctctct CT/Δ ggaccaggctgggagcagtc | 1740 | | |
| ALDH7 | 11 | intron6 + 263 | cagaccctcatacgtgaccc T/C gctgccccccaggctcttag | 1741 | | |
| ALDH7 | 12 | intron6 + 1298 | gtagacagagctggactcca T/G ccttgggtgataagggatcc | 1742 | | |
| ALDH7 | 13 | intron6 + 1411 | gccagggtcacaagcagagg C/T gggaggagccaaggggtttg | 1743 | | |
| ALDH7 | 14 | exon7 + 185 | acctgcgtggcccccgacta C/T gtcctatgcagccctgagat | 1744 | | |
| ALDH7 | 15 | exon7 + 339 | tgcgggcattgctgggctgc G/A gcgtgtggccattgggggcc | 1745 | | |
| ALDH7 | 16 | intron7 + 249 | ccagggctccagggctcagc G/A tgctaagatgaactcccatc | 1746 | | |
| ALDH7 | 17 | intron7 + 277 | atgaactcccatcccaccac C/T ggctatcctgaaaggctgta | 1747 | | |
| ALDH7 | 18 | intron7 + 498 | gaccaaggtcgggggattct C/T tgtgtcccacaggccctgag | 1748 | | |
| ALDH7 | 19 | intron8 + 14 | cagccaggtgggggtgcggc C/T gggctgggcagggtcaggag | 1749 | | |
| ALDH7 | 20 | intron8 + 49 | caggagcccgcagtgggcag C/T acaagtggtggcagcagggg | 1750 | | |
| ALDH7 | 21 | intron8 + 111 | tcaggactttgggatggtgg A/T cctcttggctctgtctctgc | 1751 | | |
| ALDH7 | 22 | intron8 + 3219 | atcctgatggggctcaaggc A/G gcctcacgcacatcctgttc | 1752 | | |
| ALDH7 | 23 | exon9 + 33 | gtgctgacccagaccagcag G/T gggggcttctgtgggaacga | 1753 | | |
| ALDH7 | 24 | intron9 + 948 | tcccaggcccccgagctgac C/A cttcttggtggccgtggccc | 1754 | | |
| ALDH7 | 25 | intron9 + 1067 | aggctccccaagcctgggtc C/T ctcttgcccccacccactct | 1755 | | |
| ALDH7 | 26 | exon10 + 137 | ccgcaatcgccgcgccgcct G/A aggatgctgctggtggccat | 1756 | | |
| ALDH7 | 27 | exon10 + 397 | cgctcccaaccatgagagcc G/A aggatgctgctggtggccat | 1757 | | |
| ALDH7 | 28 | exon10 + 1198 | ctcttccccatgctgctcat C/T ctcctgggccccatccactc | 1758 | | |
| ALDH7 | 29 | exon10 + 1475 | caggggtggacctgagtttc G/A tctcctgtctctctggctga | 1759 | | |
| ALDH7 | 30 | 3′flanking + 15 | cctggcaatacttacatctc A/G gtgatttgctttctgtgcat | 1760 | | |
| ALDH7 | 31 | 3′flanking + 60 | caacaggactctggaccaag G/C ccctggcgttgggtaacaat | 1761 | | |
| ALDH8 | 1 | intron1 + 98 | agggaaggggatgtgtgccc G/A tggccgtgggtcaggggggc | 1762 | | |
| ALDH8 | 2 | intron1 + 157 | atggctgcaggggccatggg T/C acggggcttgctcaggagag | 1763 | | |
| ALDH8 | 3 | intron1 + 354 | tctgtggacagacaaggatt C/G ggtcgggggcaccagggctg | 1764 | | |
| ALDH8 | 4 | intron1 + 851 | tatgacaggtccatcaggcc T/G caccttcctgtgtgtcttat | 1765 | | |
| ALDH8 | 5 | intron1 + 894 | ctcagcatctgcccccacag T/G gcttttgcacacgttggttc | 1766 | | |
| ALDH8 | 6 | intron1 − 463 | aaagaaccctccgagtccct C/G gtttagtcccagaagggagg | 1767 | | |
| ALDH8 | 7 | exon2 + 61 | gccttcaactgagggcgcac G/A cggccggccgagttccgggc | 1768 | | |
| ALDH8 | 8 | intron2 + 8 | ggacctgcataaggtgggcc A/G tggagagtgggccccggcag | 1769 | | |
| ALDH8 | 9 | intron2 + 23 | gggccgtggagagtgggccc G/C ggcaggggctggagcagcgt | 1770 | | |
| ALDH8 | 10 | intron2 + (180-181) | ttcactcctgaacactcaca (A) gccaccctgtgatgcaggct | 1771 | | |
| ALDH8 | 10 | intron2 + (180-181) | ttcactcctgaacactcaca gccaccctgtgatgcaggct | 1772 | | |
| ALDH8 | 11 | exon8 + 72 | gactacgctctcaagaacct T/G caggcctggatgaaggatga | 1773 | | |
| ALDH8 | 12 | intron8 + 375 | ctgcagcatcctaacctcac C/T gtcgcgactcaaggctgccg | 1774 | | |
| ALDH8 | 13 | intron8 + 463 | aatcacccccatggcacccc G/A accgtcactgagagggtgct | 1775 | | |
| ALDH8 | 14 | exon9 + 33 | atgctggagcggaccagcag C/A ggcagctttggaggcaatga | 1776 | | |
| ALDH8 | 15 | exon10 + 428 | aggtgtcctcactcacccca C/T cctccccaattccagccctt | 1777 | | |
| ALDH9 | 1 | exon1 + 121 | actgtgtggggtatggcggg G/A tggtggggagaatgtggtgt | 1778 | | |
| ALDH9 | 2 | intron1 + 67 | cgcggatttcccggccagcc C/G ccgtttcctgtgttctgcag | 1779 | | |
| ALDH9 | 3 | intron1 + 103 | tgcagcgttgacttgagcac A/G agacagtgacagtggagagt | 1780 | | |
| ALDH9 | 4 | intron1 + 1818 | gaatttttgagaaaaaaaaa A/Δ tgttcctttagggttgcctt | 1781 | | |
| ALDH9 | 5 | intron2 + 5891 | tcaggaacaggaagtaaaga G/A gtttccatttctaaatttct | 1782 | | |
| ALDH9 | 6 | intron2 + 6398 | atcaaaaacacttgtctgat T/G atcgtgctctgaacctgcct | 1783 | | |
| ALDH9 | 7 | intron2 + 9677 | atgacgctgagtttggtgct A/G ttcttttgtttttcttgcct | 1784 | | |
| ALDH9 | 8 | intron2 + 9991 | gggagaagtgagggacctac C/T cttggcttctaatctttcat | 1785 | | |
| ALDH9 | 9 | intron2 + 10198 | ttgtcagagacatctttgat A/G atccttacgtactatatcag | 1786 | | |
| ALDH9 | 10 | intron2 + 10258 | ttagtagataactttttttt T/Δ gtaaggatggagaataatag | 1787 | | |
| ALDH9 | 11 | intron2 + 11382 | catattcaattcttttatgt T/C ctttagaccaaagaaaggca | 1788 | | |
| ALDH9 | 12 | intron2 + 11455 | taaacctttaagctcatcat C/T ggaccatctattgaatttct | 1789 | | |
| ALDH9 | 13 | intron2 + 12044 | atttaaagtgaaagctattt C/T tagttttaaaaattgagcag | 1790 | | |
| ALDH9 | 14 | intron3 + 334 | ctatttagcaaacttttttt T/Δ gacagtgtataaagttttca | 1791 | | |
| ALDH9 | 15 | intron3 + 368 | gttttcaacaattgatattg G/Δ aaggttggtagggcctagga | 1792 | | |
| ALDH9 | 16 | intron4 + 191 | ccctcaaggagcttatagtt T/A aggttgtacacaatcatgtc | 1793 | | |
| ALDH9 | 17 | intron4 + 557 | tagaaaaaattgtaatgtta A/G aaagcattactgttaggaca | 1794 | | |
| ALDH9 | 18 | intron5 + 830 | agttcaagatgattttgtag G/C ttcagggcctagttgactta | 1795 | | |
| ALDH9 | 19 | intron5 + 838 | atgattttgtaggttcaggg C/T ctagttgacttagcatgcaa | 1796 | | |
| ALDH9 | 20 | intron6 + 120 | agaaaagttgcacaaatagt A/C caaagaattcccatgtacct | 1797 | | |
| ALDH9 | 21 | intron6 + 2569 | attaaaatctgctttaaata T/C ttttttgggggagaggacac | 1798 | | |
| ALDH9 | 22 | intron7 + 1414 | ccgatcttcaaaaaattagc T/C gggggtggtggtgcacactg | 1799 | | |
| ALDH9 | 23 | intron8 + 664 | aaagttcacatttttttttt T/Δ ataacttcatggtcaagagc | 1800 | | |
| ALDH9 | 24 | intron8 + 2170 | taatgcacacattttttttt T/Δ cttcatagggacatccaacg | 1801 | | |
| ALDH9 | 25 | exon11 + 587 | aaaacaaaaaacaaaaaaaa A/Δ ccttgttcctttataggttc | 1802 | | |
| ALDH10 | 1 | intron1 + 39 | gggtgtggggaaactggccc C/T cgccgcgcacttgtggactg | 1803 | | |
| ALDH10 | 2 | intron3 + 2491 | tgccgcgaagaaattggcac T/A gctgagttctacatgcagtt | 1804 | | |
| ALDH10 | 3 | intron3 + 2595 | ttctgtacatcaacttgtga T/A ggattgaggccagttctggt | 1805 | | |
| ALDH10 | 4 | intron3 + 2775 | taccgctttgcccctgacca G/A gggtaaattcttcaataact | 1806 | | |
| ALDH10 | 5 | intron3 + 3424 | aggcacttctgcacacaccc G/A cgtctcatgcattttccctg | 1807 | | |
| ALDH10 | 6 | intron3 + 3676 | atgttgaagagattgctgat G/A ttagacgttcggatttattt | 1808 | | |
| ALDH10 | 7 | intron4 + 481 | tagaaaataagaggtttcag G/T ttctctctgctaaatccggt | 1809 | | |
| ALDH10 | 8 | intron4 + 769 | atcctgctttatacctgaac G/A tcttgcaggcagagccaaaa | 1810 | | |
| ALDH10 | 9 | intron4 + 796 | aggcagagccaaaagccaca A/G ccaggagagtctgtaccgaa | 1811 | | |
| ALDH10 | 10 | intron5 + 254 | attagttgtggcatatactt T/G ttttaaaaaagttaaataat | 1812 | | |
| ALDH10 | 11 | intron6 + 137 | aatcctgctttctggtatac T/C gtacctgtagcttttgttat | 1813 | | |
| ALDH10 | 12 | intron6 + 923 | aggctaatgaatggtaagag G/A aaggggctatcctgattagc | 1814 | | |
| ALDH10 | 13 | intron7 + 331 | tgcttttctgatgttaatcc A/Δ cagggcattgctgaataaca | 1815 | | |
| ALDH10 | 14 | intron8 + 643 | tttagaacatgacctgcctg C/T ctctcccacatgtgagatga | 1816 | | |
| ALDH10 | 15 | intron8 + 666 | ctcccacatgtgagatgact G/A actcagctttttatttctcc | 1817 | | |
| ALDH10 | 16 | intron9 + 2129 | tgttttcatttttaaaaaaa G/T gtttgactttggaattcatg | 1818 | | |
| ALDH10 | 17 | exon10 + (1894-1895) | ttggcttgtctactaataca CA/Δ tctgcttcaaaatgaacata | 1819 | | |
| ALDH10 | 18 | 3′flanking + 31 | gtatttgtcaactttttttt T/Δ ctcattttaaaattcttagc | 1820 | | |
| ALDH10 | 19 | 3′flanking + 106 | gtgtgttgggggtggtggtt G/A gtagctatagtaaataggtt | 1821 | | |
| ALDH10 | 20 | 3′flanking + 1630 | aaaagcacgtgggaaacaca A/G ttaatcatgtcttaccgtat | 1822 | | |
| ABCC7 | 1 | 5′flanking − 834 | gctaaaacactccaaagcct T/G ccttaaaaatgcgcactggg | 1823 | | |
| ABCC7 | 2 | 5′flanking − 729 | cctccttgcagatttttttt T/Δ ctctttcagtacgtgtccta | 1824 | | |
| ABCC7 | 3 | exon1 + 125 | tagcagggaccccagcgccc G/C agagaccatgcagaggtcgc | 1825 | | |
| ABCC7 | 4 | intron1 + 5200 | ctatgtgagacgttaagaag G/A tagaggtggccaagaaggaa | 1826 | | |
| ABCC7 | 5 | intron1 + 7538 | agttctctttcttagcatgg C/A ctacagaggtgcaactacct | 1827 | | |
| ABCC7 | 6 | intron1 + 13519 | gaaacttaaatcttgagtca T/C acaattgtgtctacatactg | 1828 | | |
| ABCC7 | 7 | intron1 + 14110 | attacacagtattttttttt T/Δ ctctttcagtacgtgtccta | 1829 | | |
| ABCC7 | 8 | intron1 + 14293 | gccaggcagattcctgactc C/Δ tataacccagagcttatcag | 1830 | | |
| ABCC7 | 9 | intron1 + 14316 | taacccagagcttatcagag C/G atttatgtccccaaagagaa | 1831 | | |
| ABCC7 | 10 | intron1 + 14433 | cagaataacaatgatggctc G/A gaaaaatatgggtatttctg | 1832 | | |
| ABCC7 | 11 | intron1 + 14824 | acgttttgacagttgcacaa G/C tttctttctttaagctttaa | 1833 | | |
| ABCC7 | 12 | intron1 + 23401 | aatatttttgaaaatcacta C/G ggtatcctgcatagtgattt | 1834 | | |
| ABCC7 | 13 | intron3 + 879 | gaaaaatttcagttcataca C/A ccccatgaaaaatacattta | 1835 | | |
| ABCC7 | 14 | intron3 + 922 | acttatcttaacaaagatga G/C tacacttaggcccagaatgt | 1836 | | |
| ABCC7 | 15 | intron3 + 933 | caaagatgagtacacttagg C/T ccagaatgttctctaatgct | 1837 | | |
| ABCC7 | 16 | intron3 + 13704 | tttttccaaataaaaaaaaa A/Δ tcaggtgatatctgtaaatg | 1838 | | |
| ABCC7 | 17 | intron3 + 13758 | tattaaagaacatgatgctt A/G aaacagattagggaaaacta | 1839 | | |
| ABCC7 | 18 | intron4 + 240 | ctctgttgtagttttttttt T/Δ ctcctaatcatgttatcatt | 1840 | | |
| ABCC7 | 19 | intron4 + 376 | ttatgttcagcaagaagagt A/G taatatatgattgttaatga | 1841 | | |
| ABCC7 | 20 | intron4 + 586 | tgtccagacaagagaccaaa T/C tgccgaggcatcatttaggt | 1842 | | |
| ABCC7 | 21 | intron4 + 1089 | tttcaatctgaacattttac G/A taagtgaagactttgttaga | 1843 | | |
| ABCC7 | 22 | intron4 + 1615 | aaagttaggtggtattgtat C/T tgtcttcctttctcaatgtt | 1844 | | |
| ABCC7 | 23 | intron4 + 1946 | aatacaaacaaacttgagct T/C tgcctatacttttcaagaat | 1845 | | |
| ABCC7 | 24 | intron6 + 783 | tatctaagttttggagtcaa A/G tagcactttgtttgaatccc | 1846 | | |
| ABCC7 | 25 | intron6 + (1128-1131) | gattgattgattgattgatt GATT/Δ tacagagatcagagagctgg | 1847 | | |
| ABCC7 | 26 | intron7 + (731-732) | gtagcaatgagaccattttt (T) cttcagttgagctccatgtt | 1848 | | |
| ABCC7 | 26 | intron7 + (731-732) | gtagcaatgagaccattttt cttcagttgagctccatgtt | 1849 | | |
| ABCC7 | 27 | intron7 + 1434 | gaatgtttggttgtaacctg T/C ataatctggcatgaaattgt | 1850 | | |
| ABCC7 | 28 | intron1 + 752 | catgctctcttctcagtccc A/G ttccttcattatatcaccta | 1851 | | |
| ABCC7 | 29 | intron8 + 1109 | tatggccaagacttcagtat G/A cgtggacttaattcttcctt | 1852 | | |
| ABCC7 | 30 | intron8 + 1312 | atgaagacattcattttttt T/Δ ctccgtccaatgttggatta | 1853 | | |
| ABCC7 | 31 | intron9 + (6521-6522) | gtgtgtgtgtgtgtgtgtgt (GT) ttttttaacagggatttggg | 1854 | | |
| ABCC7 | 31 | intron9 + (6521-6522) | gtgtgtgtgtgtgtgtgtgt ttttttaacagggatttggg | 1855 | | |
| ABCC7 | 32 | intron10 + 2119 | gaacactttatagttttttt T/G ggacaaaagatctagctaaa | 1856 | | |
| ABCC7 | 33 | intron11 + 3867 | tttttcttcaagaaattaga A/Δ gaggggagaaattggtttaa | 1857 | | |
| ABCC7 | 34 | intron11 + 11844 | tgaatcaaaatcatctaaaa A/Δ gctttcagaaaccagacttt | 1858 | | |
| ABCC7 | 35 | intron11 + 12144 | atattaaacagagttacata T/C acttacaacttcatacatat | 1859 | | |
| ABCC7 | 36 | intron11 + 20975 | gtgtggatagtaaatgccag G/A gtaaatcacatagcatctaa | 1860 | | |
| ABCC7 | 37 | intron11 + 27057 | atggaagagaagttttagta G/A aggggaggaaggaggaggtg | 1861 | | |
| ABCC7 | 38 | intron11 + 27131 | gagagagacttttttttttt T/Δ aaggcgagagtttactacct | 1862 | | |
| ABCC7 | 39 | intron13 + 152 | gtattaactcaaatctgatc T/A gccctactgggccaggattc | 1863 | | |
| ABCC7 | 40 | intron13 + 287 | tttgcagtatcattgccttg T/C gatatatattactttaatta | 1864 | | |
| ABCC7 | 41 | intron15 + (85-86) | atacatatatatgcacacac AT/Δ aaatatgtatatatacacat | 1865 | | |
| ABCC7 | 42 | intron15 + 106 | taaatatgtatatatacaca T/A gtatacatgtataagtatgc | 1866 | | |
| ABCC7 | 43 | intron15 + 3341 | ggaagtataaatttgtaaat A/C actgagacccaaacttacaa | 1867 | | |
| ABCC7 | 44 | intron15 + 5556 | tgctattgactaatagtaat A/T attttagggcagctttatga | 1868 | | |
| ABCC7 | 45 | intron15 + 5919 | tggtagttctatgtggaaac C/A gtgaggaaataattttatat | 1869 | | |
| ABCC7 | 46 | intron17 + 2479 | caaaaaggtatggaagtcag A/C ggagaaggagacccctatgt | 1870 | | |
| ABCC7 | 47 | intron18 + 81 | acgtatgcaaaaaaaaaaaa A/Δ gaaataactcactgacacac | 1871 | | |
| ABCC7 | 48 | intron19 + 751 | cattaataaaataacaaatc A/G tatctattcaaagaatggca | 1872 | | |
| ABCC7 | 49 | intron19 + 820 | tgacatttgtgatatgatta T/C tctaatttagtctttttcag | 1873 | | |
| ABCC7 | 50 | intron21 + 1532 | ttacctttaacttttttttt T/Δ agtttgatcagctctcttta | 1874 | | |
| ABCC7 | 51 | intron21 + 1607 | atgcttttggagttgggtct C/T ataaatgtatagaaatgttt | 1875 | | |
| ABCC7 | 52 | intron21 + 11260 | atgtggaacaatcatgacta T/C atgccttttactttctctat | 1876 | | |
| ABCC7 | 53 | intron22 + (130-131) | agaatcaatattaaacacac AT/Δ gtttattatatggagtcat | 1877 | | |
| ABCC7 | 54 | intron23 + 1828 | ctgtcctaaagtttaaaaag A/Δ aaaaaaaaaaggaagaaggaa | 1878 | | |
| ABCC7 | 55 | intron24 + (7100-7112) | cctttacaaactcttagaca (T)12-14 agtttaacatgttacaaaac | 1879 | | |
| ABCC7 | 56 | intron25 + 237 | actcttcccccttgtcaaca C/T atgatgaagcttttaaatac | 1880 | | |
| ABCC7 | 57 | intron27 + 115 | gggtgaagctctttccccac C/T ggaactcaagcaagtgcaag | 1881 | | |
| ABCC7 | 58 | intron27 + 334 | ggatgaattaagtttttttt T/Δ aaaaacgccccatttggtaa | 1882 | | |
| ABCC8 | 1 | 5′flanking − 1099 | aaaggggctgaaggggtctt T/C cttttgtgttcccctgactg | 1883 | | |
| ABCC8 | 2 | 5′flanking − (424-422) | caccccaccaccaccaccac CAC/Δ aaggtaacgttctgccccac | 1884 | | |
| ABCC8 | 3 | intron1 + 1212 | agcctgggcaacatagtgag A/G ccccccccgccctttctaca | 1885 | | |
| ABCC8 | 4 | intron2 + 1003 | aggaggactgtgaatcccag C/A ctgcatgtttgggtcggatt | 1886 | | |
| ABCC8 | 5 | intron2 + 1253 | catctcactaaggaagaatc C/T agtaaccagcaaggatgaga | 1887 | | |
| ABCC8 | 6 | intron2 + 1382 | cccagactgcactcctgcag T/C gctgcctggctcctgtagtt | 1888 | | |
| ABCC8 | 7 | intron2 + 2371 | tttcagagctgtctggaaat T/A tagggggcaggtgggagggg | 1889 | | |
| ABCC8 | 8 | intron3 + 1957 | ccctacccctagcccagggg C/T ccccacatgagtatgaatgg | 1890 | | |
| ABCC8 | 9 | intron3 + (2088-2089) | agagaacccttcattaacca (CCA) gggcgtggctgaccagtgtc | 1891 | | |
| ABCC8 | 9 | intron3 + (2088-2089) | agagaacccttcattaacca gggcgtggctgaccagtgtc | 1892 | | |
| ABCC8 | 10 | intron3 + 2204 | taaagcacaagttatcaccc G/A tggatggatttgtccttttc | 1893 | | |
| ABCC8 | 11 | intron3 + 2286 | ttatctccccttgaaaggac A/G ctccacagagccagaaattc | 1894 | | |
| ABCC8 | 12 | intron3 + 2312 | cagagccagaaattctagaa C/G agggaaaagtggaggggagg | 1895 | | |
| ABCC8 | 13 | intron3 + 2358 | ctgtgaactgcagggacaga A/G ggaaatgggtattgggagaa | 1896 | | |
| ABCC8 | 14 | intron3 + 2359 | tgacctgcagggacagaagg A/C aatgggtattgggagaatgg | 1897 | | |
| ABCC8 | 15 | intron3 + 2370 | gacagaaggaaatgggtatt G/A ggagaatggccagccctcca | 1898 | | |
| ABCC8 | 16 | intron3 + 2382 | tgggtattgggagaatggcc A/G gccctccaaggggctgatgt | 1899 | | |
| ABCC8 | 17 | intron3 + 4910 | ggggacagccttcagctgtg G/A aattcctccagtcctagaga | 1900 | | |
| ABCC8 | 18 | intron3 + 4969 | cattattccagtcctgaggc A/G tgagagcagaaggccgatgc | 1901 | | |
| ABCC8 | 19 | intron3 + 5003 | ccgatgcttctgccctccat C/G ctaatgtcctcctgcaggga | 1902 | | |
| ABCC8 | 20 | intron3 + 5019 | ccatcctaatgtcctcctgc A/C gggacccaaggtggatggca | 1903 | | |
| ABCC8 | 21 | intron4 + 14 | ggtgagggtaagcaggccac C/T tgggccagggtggggtggga | 1904 | | |
| ABCC8 | 22 | intron4 + 187 | agacactgcatctggcccac G/A tgtgctctaccccagggtcc | 1905 | | |
| ABCC8 | 23 | intron4 + 204 | cacgtgtgctctaccccagg G/C tcccagagggagaggggggt | 1906 | | |
| ABCC8 | 24 | intron4 + 254 | gttcgctgaggttggcggat G/A actttccgtagaaagggaag | 1907 | | |
| ABCC8 | 25 | intron4 + 357 | tgtattcatatcgtcacgct G/C gtaaatgaatgagtaagtgt | 1908 | | |
| ABCC8 | 26 | intron5 + 92 | ggcattaggtcaaaatcctg G/A tgggacaaaaggggaaactg | 1909 | | |
| ABCC8 | 27 | intron6 + 4205 | tctgtagaaagtacatgggg G/A catgaagatcattggcttga | 1910 | | |
| ABCC8 | 28 | intron6 + 5519 | gattcccagggaatgttaaa A/C aggaccgggtcttcctaaac | 1911 | | |
| ABCC8 | 29 | intron6 + 5575 | tctgacccagtaccagccag G/C gggcaagtttccatcccccc | 1912 | | |
| ABCC8 | 30 | intron6 + 5587 | gttgccatctgagatcttgc C/T ggaagtacacaagagaccct | 1913 | | |
| ABCC8 | 31 | intron6 + 6747 | ttccactggccttttctgct C/T agtaattgctacattacagg | 1914 | | |
| ABCC8 | 32 | intron9 + 191 | gaggaagctgcctcccggtg A/G ggacaggaagcgggcatggc | 1915 | | |
| ABCC8 | 33 | intron10 + 1963 | cccaggagtccaacctccct T/G tgtccagctagaccatggtg | 1916 | | |
| ABCC8 | 34 | intron10 + 2724 | cctgggacatgttttcttat A/G taaaacagcatcaaagatgt | 1917 | | |
| ABCC8 | 35 | intron10 + 2938 | gcccgcccaggactcctcac G/C tgtcaaagtcacctagggag | 1918 | | |
| ABCC8 | 36 | intron10 + 3094 | tccgaggatgtgtttttttt T/Δ ccctccgttagtcagcagtg | 1919 | | |
| ABCC8 | 37 | intron10 + 3368 | tcctgctcatatgcggcacc A/G tcagacttctgggcaggcaa | 1920 | | |
| ABCC8 | 38 | intron10 + 8897 | ggtattgattaaaagcctca C/T gggcagagaaattcgccatc | 1921 | | |
| ABCC8 | 39 | intron11 + 308 | tgtgtattgtagaagtgatg G/A gaaatccagaacagaaagct | 1922 | | |
| ABCC8 | 40 | intron11 + 1171 | gccctctcatttcccttcca G/A tgctgagcgtttccagtgtg | 1923 | | |
| ABCC8 | 41 | exon12 + 7 | gcctctgtccacagactttc G/A tgggccacgtcagcttcttc | 1924 | | |
| ABCC8 | 42 | intron12 + 356 | accaagaatgaggccatccc G/T tccccacgtggctgccccat | 1925 | | |
| ABCC8 | 43 | intron12 + 934 | tgggttcaaagatggaatgg G/T gcataactcagcaaaattat | 1926 | | |
| ABCC8 | 44 | intron12 + 1370 | gggagggaggctggacaggg C/G atgaaggcagagcctggtgg | 1927 | | |
| ABCC8 | 45 | intron15 + 412 | ggaggtgggacccaggatgg C/T gtttcttgggaccacaagga | 1928 | | |
| ABCC8 | 46 | intron15 + 688 | actcccccggccccactcac A/G tctgccaccttccctccctg | 1929 | | |
| ABCC8 | 47 | intron16 + 4464 | actcattccaagtattgatc G/A agaagagaggtaggtactgg | 1930 | | |
| ABCC8 | 48 | intron16 + 4574 | ttgaagatcttaagttgttt T/C tggttcactcatttcgcaaa | 1931 | | |
| ABCC8 | 49 | intron16 + 5011 | agctaaaagcaaaacagcct C/T tgacctggcaagcattccca | 1932 | | |
| ABCC8 | 50 | intron16 + 7608 | tgtcctacttttcttttgac C/G cttataacttcctgacttcg | 1933 | | |
| ABCC8 | 51 | intron16 + 7730 | ccagctcctagtgggctgga G/A ggaaggacatgcggttgggg | 1934 | | |
| ABCC8 | 52 | intron16 + 8369 | ttgcaaactgagttagggcc T/C ggagagcttactgtgtgctg | 1935 | | |
| ABCC8 | 53 | intron16 + 9708 | tgcacttgccgcctacttat T/G ccagacccaatgattgggtc | 1936 | | |
| ABCC8 | 54 | intron17 + 651 | tatagattaatgaggctctg A/G gtccctcaaaaccttccctc | 1937 | | |
| ABCC8 | 55 | intron17 + 692 | cccttacctctccaaaaaac A/G cttgagataccctagaggtg | 1938 | | |
| ABCC8 | 56 | intron17 + 1541 | ctcaggatcttcctggagga C/T atggttcactcccatgagag | 1939 | | |
| ABCC8 | 57 | intron18 + 580 | actaagcagatttctaccaa C/T tgcacctccccatccccttg | 1940 | | |
| ABCC8 | 58 | intron18 + 658 | gaacaagcccctgagaatgc C/T ttccgcaccccctactcccg | 1941 | | |
| ABCC8 | 59 | intron18 + 660 | acaagcccctgagaatgcct T/C ccgcaccccctactcccgcc | 1942 | | |
| ABCC8 | 60 | intron19 + 93 | gcccttccatcgatcaccca T/C acccagccatctcactcccc | 1943 | | |
| ABCC8 | 61 | intron19 + 123 | tctcactccccaggtgctta T/C ctgcactccagcctctccat | 1944 | | |
| ABCC8 | 62 | intron19 + 219 | cataggggagagggcaggaa C/T ggagggaagggagagagccc | 1945 | | |
| ABCC8 | 63 | intron19 + 845 | tagtatttaacctgcccaaa C/T gctgtgtgaagtgctgacct | 1946 | | |
| ABCC8 | 64 | intron20 + 338 | tcccctccacaagcttagac A/G aacaggattctcctgtgact | 1947 | | |
| ABCC8 | 65 | exon21 + 10 | tttggtgacagggcatcaac C/T tgtctggtggtcaacgccag | 1948 | | |
| ABCC8 | 66 | intron21 + 192 | caaggatagcacaaatgacc C/Δ attgcagacttcagatggag | 1949 | | |
| ABCC8 | 67 | intron23 + 17 | gaaggtgggtatatccaggg A/G tggccaagcagccacccctg | 1950 | | |
| ABCC8 | 68 | intron23 + 67 | gttctgctagaacctgaact G/T ataaaggtcttcctgtcctt | 1951 | | |
| ABCC8 | 69 | intron26 + 268 | gtgagcgtctgcacatccaa G/C taaagattgttttctcctcc | 1952 | | |
| ABCC8 | 70 | intron26 + 308 | cgataagtgggtgtaatttg C/T ccatccccacccatgagtcc | 1953 | | |
| ABCC8 | 71 | intron26 + 348 | cagctccctgccctcccctc A/G ctctctctccctcagccagc | 1954 | | |
| ABCC8 | 72 | intron26 + 807 | gacagctgctgagtcaggcc G/A agccggcagctgagaaaggc | 1955 | | |
| ABCC8 | 73 | intron26 + 834 | cagctgagaaaggcggcagt G/C gtcagatgggcttgagaaac | 1956 | | |
| ABCC8 | 74 | intron26 + (118-121) | cctccaaaaaataaaaacaa AAAA/Δ cagaaatgaaggaaatagaa | 1957 | | |
| ABCC8 | 75 | intron28 + 1348 | tggggtaagcggaagacggg G/A ttgaacgctttgagtttggt | 1958 | | |
| ABCC8 | 76 | intron29 + 1253 | ctcttagggatcttgtctaa G/T taaagaagagcagagcaaag | 1959 | | |
| ABCC8 | 77 | intron29 + 1589 | cagatcccagcttcctgtaa A/G cagcctcagatcaggccaaa | 1960 | | |
| ABCC8 | 78 | intron29 + 2322 | gcgcctcacactcctataac G/A cgcacatgccctgatgcaca | 1961 | | |
| ABCC8 | 79 | intron29 + 2348 | atgccctgatgcacacacat T/C ttcaacacgcacttactcta | 1962 | | |
| ABCC8 | 80 | intron29 + 2418 | agacacgtcaccctcccaca C/T gtctccaccctgggggtgtg | 1963 | | |
| ABCC8 | 81 | intron29 + 2494 | tcagtcccctcagacacatg C/A cctctctccacgcagagaca | 1964 | | |
| ABCC8 | 82 | intron29 + 2735 | gcggccaaggagagtgatga C/T ggcagcccaggttgatcaga | 1965 | | |
| ABCC8 | 83 | intron30 + 386 | gctcctggggctccagcctt C/T gcagcccttgtgtgtgtctg | 1966 | | |
| ABCC8 | 84 | intron33 + 93 | ggcttcgcagtcacctcgtg G/T ccctccagggccgaggcctc | 1967 | | |
| ABCC8 | 85 | intron33 + 358 | agggacctgggggcagacag C/T gaggccacccttgtattgag | 1968 | | |
| ABCC8 | 86 | intron38 + 54 | cccagggacaggactggcct G/C ttgtggccgtcatcagtgca | 1969 | | |
| ABCC8 | 87 | intron38 + 466 | aggacattctggccacatgc C/Δ tcatcctcctcctccaagcc | 1970 | | |
| ABCC8 | 88 | intron38 + 529 | tggcccccaccgcgggtggt A/G ttcccaccatcctgacccgc | 1971 | | |
| ABCC9 | 1 | intron3 + 38 | tgttgtttctccttaaagag C/A tatttgtttttccccccaaa | 1972 | | |
| ABCC9 | 2 | intron3 + 305 | gctggccttctggcttgcag T/A agttgtattttaagaatcag | 1973 | | |
| ABCC9 | 3 | intron3 + 320 | tgcagaagttgtattttaag A/G atcagagctcttgtgaggag | 1974 | | |
| ABCC9 | 4 | intron3 + 631 | ttctgtggaaatcagaggct G/C tctaaaatattcctaatttt | 1975 | | |
| ABCC9 | 5 | intron3 + 8644 | tggacgactcaacatttttc A/G agttattactccttcaactc | 1976 | | |
| ABCC9 | 6 | intron4 + 757 | aggatatcatgaaacactga A/C tcttagtaaaaactatcttt | 1977 | | |
| ABCC9 | 7 | intron4 + 1022 | tactgtggaatttttcttgc A/C acagagatatgtatttttca | 1978 | | |
| ABCC9 | 8 | intron5 − 1217 | cagtggtagatgtgttttct A/G ttgccatcatctacaaatat | 1979 | | |
| ABCC9 | 9 | intron6 + (106-107) | tatgagttgttcaaataggc (T)7-9 cagagaattgaatgctttct | 1980 | | |
| ABCC9 | 10 | intron6 + 1347 | tcagtcgtattcctactaaa A/Δ caaaattttgtaagttatgt | 1981 | | |
| ABCC9 | 11 | intron6 + 1618 | ctttttatttgctgcttacc G/A ttttactaaggttggatata | 1982 | | |
| ABCC9 | 12 | intron6 + 1835 | cttttaataaatgcaaactg C/T acacctggtctataaaaaga | 1983 | | |
| ABCC9 | 13 | intron7 + 407 | cctatagaatttttcttttc T/G tttttctcaaaaaaattaaa | 1984 | | |
| ABCC9 | 14 | intron7 + 423 | tttctttttctcaaaaaaaa C/T taaatgtttgttatttattt | 1985 | | |
| ABCC9 | 15 | intron3 + 743 | ttctgtagatgaagcttaag A/T gctagatcttatttgaaaaa | 1986 | | |
| ABCC9 | 16 | intron8 + 850 | tttttaacttattgtttgcc T/G tttcattttttaatagaaaa | 1987 | | |
| ABCC9 | 17 | intron9 + 585 | cgaatttgctgcttttagag A/T aatctttgcaaataataaaa | 1988 | | |
| ABCC9 | 18 | intron9 + 1394 | atttttcttcttgtaagtat G/C agtgatagagctgactgcag | 1989 | | |
| ABCC9 | 19 | intron12 + 1167 | atttgtaagacttttaaaat G/A agataattgtgctggtgtct | 1990 | | |
| ABCC9 | 20 | intron12 + 1195 | tgtgctggtgtctatatctt A/G ctgagaaaactagaatttat | 1991 | | |
| ABCC9 | 21 | intron12 + 2123 | ataagtgctctcccagtgtt G/A attggacttagagcattttc | 1992 | | |
| ABCC9 | 22 | intron12 + (2853-2856) | caaaacagaataatgaaaag TAAC/Δ tattatctaaaataataaaa | 1993 | | |
| ABCC9 | 23 | intron13 + (3043-3044) | aacatactctcctcctctct (CTCTTT) aagtcaaaatatattagtat | 1994 | | |
| ABCC9 | 23 | intron13 + (3043-3044) | aacatactctcctcctctct (CT) aagtcaaaatatattagtat | 1995 | | |
| ABCC9 | 23 | intron13 + (3043-3044) | aacatactctcctcctctct aagtcaaaatatattagtat | 1996 | | |
| ABCC9 | 24 | intron14 + 85 | ttctgtgaaagtgtcccaaa T/A tgtgcctttaaattgttttt | 1996 | | |
| ABCC9 | 25 | intron14 + 275 | agtgtcacagtgtatttttc T/C ggtattcctatgtttatcaa | 1997 | | |
| ABCC9 | 26 | intron14 + 453 | ctcattcaaaacttggctat T/C tggactctccccaggcattg | 1998 | | |
| ABCC9 | 27 | intron14 + 3709 | atcccctagtgatgtacact G/A agcttgcctccatctttcct | 1999 | | |
| ABCC9 | 28 | intron14 + 3813 | ctgatttatatattagctga C/T tttccaagttcagacatcta | 2000 | | |
| ABCC9 | 29 | intron14 + 4000 | ttcttttacttcaatgtagc A/Δ ccaaatcagaaggtgacatt | 2001 | | |
| ABCC9 | 30 | intron16 + 1466 | atcccactggatttaattac A/C ttgtgtagcttgtacaacca | 2002 | | |
| ABCC9 | 31 | intron16 + 5357 | attttggaagagaaattata T/G aaccttccacaactgaattt | 2003 | | |
| ABCC9 | 32 | intron17 + 1368 | aatcctggtgtttttttttt T/Δ ctttttcatttttcagtagg | 2004 | | |
| ABCC9 | 33 | intron20 + 98 | aagtaactcaaggaaagatg G/A tttaacttgtgaaatcgtaa | 2005 | | |
| ABCC9 | 34 | intron22 + 28 | ctcatagttcagaagagttc A/C gagcccaattcagaagagtt | 2006 | | |
| ABCC9 | 35 | intron22 + 194 | tgaacctataaaattctaat G/Δ ccatctttggatgaggtgca | 2007 | | |
| ABCC9 | 36 | intron22 + 1370 | ccagggacaaaagaagatga C/T gtaaacttaaggattgggac | 2008 | | |
| ABCC9 | 37 | intron22 + 1487 | agcaagccaggaagaaagtc C/G attaagttgtatttagaaat | 2009 | | |
| ABCC9 | 38 | intron23 + (455-462) | atagccatgaaggataagaa AATTAGAA/Δ tgccatttgt | 2011 | | |
| | | | tatgtttcag | | | |
| ABCC9 | 39 | intron24 + (460-465) | aactctttctcttcatctgc TTTAAAA/TTTTAA gcaagccttg | 2012 | | |
| | | | aaggagagtg | | | |
| ABCC9 | 40 | intron24 + 595 | gcatgcaaaataatgaagaa A/G acaatcttgtctgacattga | 2013 | | |
| ABCC9 | 41 | intron28 − 926 | aaatatttcagaatttgggg G/A tgtagagcatttgccgtcat | 2014 | | |
| ABCC9 | 42 | intron29 + 2692 | cttgtaagtctttttttttt T/Δ aaagtaatgaaaattctaa | 2015 | | |
| ABCC9 | 43 | intron29 + 5464 | agacaacactgcttttttgt G/A tgttcacaattcaacgacag | 2016 | | |
| ABCC9 | 44 | intron29 − 1830 | aactggctgaaaggaaaaaa A/T tcatattgctgtaaatattt | 2017 | | |
| ABCC9 | 45 | intron31 + 102 | tgcttttgctttccacttca G/A tatccagaaaactctctcat | 2018 | | |
| ABCC9 | 46 | intron33 + 877 | aacatggaactatagtaaat A/G tagtttttttggggttcaga | 2019 | | |
| ABCC9 | 47 | intron36 + 1281 | aatttacacttttttttttt T/Δ gcaggagaatattttgcaaa | 2020 | | |
| ABCC9 | 48 | 3′flanking + 197 | aatggagctcatgcatgtgt T/G ttcaaatatatacatgcaaa | 2021 | | |
| CES1 | 1 | 5′flanking − 983 | tatttccttagccagcggta T/C cacagtgtgtttagtgaatt | 2022 | | |
| CES1 | 2 | 5′flanking − 814 | tcacattgccttgacatcac A/C cctactgctcctccacccta | 2023 | | |
| CES1 | 3 | 5′flanking − 248 | agtcctgcaagggtgacacc G/Δ ttatgccacaagcagttggg | 2024 | | |
| CES1 | 4 | intron1 + 22 | tgagtccttctgaagtcaaa T/Δ atgcggggcactttttgaaa | 2025 | | |
| CES1 | 5 | intron1 + 30 | tctgaagtcaaatatgcggg G/T cactttttgaaatccttgtt | 2026 | | |
| CES1 | 6 | intron1 + 1662 | aagggaatccctgagctgag C/A atgaccagcccagtggtttc | 2027 | | |
| CES1 | 7 | intron1 + 1726 | cctccctgaagtcctcagca A/C tcttagctggttcctcgccc | 2028 | | |
| CES1 | 8 | intron1 + 2716 | tgcttccaaggaagttcatc T/G cagtattatttgtaattagc | 2029 | | |
| CES1 | 9 | intron1 + (2747-2749) | tgtaattagcaacaacaaca AAA/Δ gaaaagaagctaaatattga | 2030 | | |
| CES1 | 10 | intron1 + 3288 | ttatttgtccattaaagaaa A/Δ ctcaagcgcttagcctggca | 2031 | | |
| CES1 | 11 | intron1 + 3691 | gagaatatgggacacccctt T/G ttcatcctctcatccagcat | 2032 | | |
| CES1 | 12 | intron1 + 3819 | tccttcttgcatttattttt A/G gctggatgtttttatgcctc | 2033 | | |
| CES1 | 13 | intron1 + 3880 | aaccagctcaatgggttagg G/A aggacattgatcgtcatccc | 2034 | | |
| CES1 | 14 | intron2 + 74 | gagtcaaggcagtcccctga T/C gggctgatcctttgctctgg | 2035 | | |
| CES1 | 15 | intron2 + 552 | atggaaggtgtgtccattca C/A cctggccaagctgggaagaa | 2036 | | |
| CES1 | 16 | intron2 + 885 | cagtattttagatggtaaag T/C attatgatgtaatatattgt | 2037 | | |
| CES1 | 17 | intron2 + 2001 | ttggcatgtcagggctgcaa G/A actcatgtagaaatcactcc | 2038 | | |
| CES1 | 18 | intron3 + 2119 | cgctgagtgcatgaatagtc T/C aggcttgagggtgatgggag | 2039 | | |
| CES1 | 19 | intron4 + 127 | taaggcatccaagccccttc G/A taattggacactacctaccc | 2040 | | |
| CES1 | 20 | intron4 + 347 | tctgtcatgacacttagcag T/G cagcccagcaggtgaaggtt | 2041 | | |
| CES1 | 21 | intron4 + (1984-1985) | tgtggtcctgaaggtcctgc (C) tgacatctctgctccccacc | 2042 | | |
| CES1 | 22 | intron4 + (1984-1985) | tgtggtcctgaaggtcctgc tgacatctctgctccccacc | 2043 | | |
| CES1 | 22 | intron5 + 766 | gaggtgggcagagggtcagc T/C cactactggattcctcagtc | 2044 | | |
| CES1 | 23 | intron5 + 825 | ggagtagatctagcctggaa T/G agcgagtgagtcactgaccc | 2045 | | |
| CES1 | 24 | intron5 + 828 | gtagatctagcctggaatag C/T gagtgagtcactgaccccac | 2046 | | |
| CES1 | 25 | intron5 + 868 | ctcctgagcatgaactctcc T/A cccctccactctgctgtcag | 2047 | | |
| CES1 | 26 | intron7 + 68 | acttcttcatttcagctgtc C/G tcttgcccagggacagtttc | 2048 | | |
| CES1 | 27 | intron7 + 681 | cctccaaaatcaacaatcca A/G ttatcgcctgtctgctagtt | 2049 | | |
| CES1 | 28 | intron7 + 885 | aggaactatccaaagagaaa T/C acattcatatacttcgcagg | 2050 | | |
| CES1 | 29 | intron7 + 2151 | gtcgtgtaaactgaaaatct C/G aggagttgatggcttcaggc | 2051 | | |
| CES1 | 30 | intron7 + 2470 | atatagatatacgaattcac G/A gagtgatgcgggaagaacct | 2052 | | |
| CES1 | 31 | intron8 + 128 | cgtgtttgtttctgaggccc A/C gagaggggtagtgactcacc | 2053 | | |
| CES1 | 32 | intron8 + 2618 | cctgatggcaacacatgagt T/C gggctctctctaatctgtga | 2054 | | |
| CES1 | 33 | intron8 + 2665 | aaaaattattcatcaaaggt G/A aaacctaaaattaagacatg | 2055 | | |
| CES1 | 34 | intron8 + 3785 | ccatggcgcatggccatgcc G/A gtctatggtactggtctcac | 2056 | | |
| CES1 | 35 | intron8 + 3791 | cgcatggccatgccggtcta T/C ggtactggtctcaccctcag | 2057 | | |
| CES1 | 36 | intron10 + 222 | gtgggctggagaagctgcat C/T gctcacccggggctggtggt | 2058 | | |
| CES1 | 37 | intron10 + 230 | gagaagctgcatcgctcacc A/C ggggctggtggtcacttttt | 2059 | | |
| CES1 | 38 | intron11 + 1177 | ctagcaggtgccctgacaca C/G ctttgcacaggaaggggcag | 2060 | | |
| CES1 | 39 | intron11 + 1311 | gccctatgctctgcgtctga A/G ctatatatagagttcccatc | 2061 | | |
| CES1 | 40 | intron11 + 2025 | ttctcatttgggatgctaag A/G ttaaaaattagcataacact | 2062 | | |
| CES1 | 41 | intron11 + 2029 | catttgggatgctaagatta A/C aaattagcataacacttcca | 2063 | | |
| CES1 | 42 | intron11 + 2317 | cattcacaaaagctctttct T/C ctatggttggctctgagttt | 2064 | | |
| CES1 | 43 | intron11 + 3887 | caaatatttggctctaattc C/T gcttccacctcagacagcta | 2065 | | |
| CES1 | 44 | intron12 + 2311 | gcgcctctgggcatctcact G/A tgcatgcttaggcgccttgc | 2066 | | |
| CES1 | 45 | intron12 + 2331 | gtgcatgcttaggcgccttg C/G ggctctgttgtttttcagaa | 2067 | | |
| CES1 | 46 | 3′flanking + 71 | aacggtgatgaaagaggcga T/C gtgagaaggaaggtggcttt | 2068 | | |
| CES1 | 47 | 3′flanking + 362 | ttgcatggcacttactgacc G/A ttgcacaggcctgcaacacc | 2069 | | |
| CES1 | 48 | 3′flanking + 581 | atttctggattctgttagta C/T gtagaaagctctaaagcatg | 2070 | | |
| CES1 | 49 | 3′flanking + 1348 | aaatctgctgctgggagaga G/C agcaaagcatgcagatcaac | 2071 | | |
| ABCB4 | 33 | intron22 + 767 | acagtgggctgatgcataga A/Δ cctgtagcaatccaccagca | 2072 | | |
| AADA | 23 | intron2 + 46 | tgtcactgaggtagttcgca A/G acattttactaagtcttcag | 2073 | | |
| AADA | 24 | 3′flanking + 208 | aatgctaaaaaaaaaaaaaa A/Δ tcactgtggtactttgggga | 2074 | | |
| ABCA4 | 1 | 5′flanking − 1005 | tgccatcataagcagaaact A/C tctctctcttcttggaagct | 2075 | | |
| ABCA4 | 2 | 5′flanking − 819 | gtctagagtctttcaaagag A/T acacattctgagatttgagg | 2076 | | |
| ABCA4 | 3 | 5′flanking − 680 | agcaccaccccattgcaggg C/A tggaatgacagtaatgggcc | 2077 | | |
| ABCA4 | 4 | intron1 + 208 | tgcccttcccaggaagatgt G/A tttctctgtcctcagccaca | 2078 | | |
| ABCA4 | 5 | intron1 + 234 | ctgtcctcagccacatgaaa A/G tcttttgcctaccgtgcctg | 2079 | | |
| ABCA4 | 6 | intron1 + 510 | agctcacgatcaagtcacag T/C ttaactggacacattatttt | 2080 | | |
| ABCA4 | 7 | intron1 + 1527 | gcttaacaaccagcataaaa G/A agagcagcatgggacacgct | 2081 | | |
| ABCA4 | 8 | intron1 + 2077 | caggactgtagctgctggcc T/C aaaatgagcccattcctgtg | 2082 | | |
| ABCA4 | 9 | intron1 + 2174 | ccctctcaatctggcctttc G/C ctggcatgggtgggcgactc | 2083 | | |
| ABCA4 | 10 | intron1 + 2246 | gctcccagggagatggagcc A/G ctcgggctgagggccttggc | 2084 | | |
| ABCA4 | 11 | intron1 + 2364 | ttctgtctggcacgcctccc G/A atggctccccacctgctacc | 2085 | | |
| ABCA4 | 12 | intron1 + 4243 | ctccctggggtatgcctgta C/G gcagttaagcgtcaaggaca | 2086 | | |
| ABCA4 | 13 | intron1 + 4287 | atgccgctctggggagggga A/C gctgagcatgattttggaag | 2087 | | |
| ABCA4 | 14 | intron1 + 4309 | ctgagcatgattttggaagc C/T ggcagaagaggctattgtga | 2088 | | |
| ABCA4 | 15 | intron1 + 4416 | tgcagcaaccgcccccgccc C/T ccgccaaaaacaaacacact | 2089 | | |
| ABCA4 | 16 | intron1 + 4996 | tttacccctggaacaggcag G/A ccaagctggctggtcccctc | 2090 | | |
| ABCA4 | 17 | intron1 + 5007 | aacaggcaggccaagctggc T/C ggtcccctccctgatacaca | 2091 | | |
| ABCA4 | 18 | intron1 + 5080 | gtgtgtggctggtttcttag C/G aagcaccatggttccaagtt | 2092 | | |
| ABCA4 | 19 | intron1 + 5152 | gggagatgaacgtaagtgga G/A ggcaggcctacaaggttgca | 2093 | | |
| ABCA4 | 20 | intron1 + 7110 | ccactggatctgcttttgga A/G tcaagagtccttaagctcca | 2094 | | |
| ABCA4 | 21 | intron1 + 7290 | gatttttgttggctttgcaa T/A ggatcacagtcatttattca | 2095 | | |
| ABCA4 | 22 | intron1 + 7483 | tctgagcctctttccttaac T/C gcagagtgagtggctacaga | 2096 | | |
| ABCA4 | 23 | intron1 + 7497 | cttaactgcagagtgagtgg C/T tacagagaaatctttactac | 2097 | | |
| ABCA4 | 24 | intron2 + 1067 | tcaagcagcagcagcaactg C/A gtggagtcttcttgaactaa | 2098 | | |
| ABCA4 | 25 | intron2 + 1106 | aacactcctatgcccctctc G/A gcacaaaatgacgtgtcccc | 2099 | | |
| ABCA4 | 26 | intron2 + 1119 | ccctctcggcacaaaatgac G/A tgtccccccttgcttcccct | 2100 | | |
| ABCA4 | 27 | intron2 + 1243 | cacccagcacagggactggc A/T cacatgagatgctcctgctt | 2101 | | |
| ABCA4 | 28 | intron3 + 26 | tgttgagatccctaccatgc A/G ggggaggaagttgcacaccc | 2102 | | |
| ABCA4 | 29 | intron3 + 101 | agcatggagcactgagtgtt C/T ttgtggctttgctgagcccc | 2103 | | |
| ABCA4 | 30 | intron3 + 330 | tgcttgggtggagtgaatca T/C tgtaggagaaaaactcagtt | 2104 | | |
| ABCA4 | 31 | intron3 + 470 | tgaagtcaggtttacaaagt C/G aagtttacttcttgggagaa | 2105 | | |
| ABCA4 | 32 | intron3 + 634 | tgaaaaccaatgacccctct T/C ccaagaaaaatggccacata | 2106 | | |
| ABCA4 | 33 | intron3 + 1016 | ccttgggggagctcagtatg A/G ttcttccaggagaagcctgc | 2107 | | |
| ABCA4 | 34 | intron3 + 1554 | gaaagttgggtttcatgttt T/C gcactcacattatgagtgaa | 2108 | | |
| ABCA4 | 35 | intron3 + 1686 | ctagacattctcacagagcc A/G agggcagcaaggcggggctc | 2109 | | |
| ABCA4 | 36 | intron3 + 1823 | ttcacctctctccatggacc A/G gtctcccctgctcctcaatg | 2110 | | |
| ABCA4 | 37 | intron3 + 1938 | caaattcctgggaacaaatc G/A ggttgacccagctttattct | 2111 | | |
| ABCA4 | 38 | intron3 + 1951 | acaaatcgggttgacccagc T/G ttattctccctgtcccatca | 2112 | | |
| ABCA4 | 39 | intron3 + 2063 | ggctgtcagagcctacctgc G/T tgaatgggtggaagggcagg | 2113 | | |
| ABCA4 | 40 | intron3 + 2079 | ctgcgtgaatgggtggaagg G/A caggtctcagagaattgggt | 2114 | | |
| ABCA4 | 41 | intron3 + 2186 | agacacacagagcatgggac C/T gagaggcgagcagaccctgc | 2115 | | |
| ABCA4 | 42 | intron3 + 2214 | gagcagaccctgccaaaact G/A ggagactgaatagatcgctc | 2116 | | |
| ABCA4 | 43 | intron4 + 2717 | cgtgcttctgcacagccacc T/C gggaaggtatgccgatggtt | 2117 | | |
| ABCA4 | 44 | intron4 + 2802 | attctcagcagggaggatta A/G tggtaaaagcccaggaatgg | 2118 | | |
| ABCA4 | 45 | intron4 + 3182 | cccccagagccacagcagcc C/G tgtctcctgggtggtcttgt | 2119 | | |
| ABCA4 | 46 | intron4 + 3515 | agtataataaaagcaggagc C/T atagcccccaactctcaaga | 2120 | | |
| ABCA4 | 47 | intron4 + 3907 | aggggagtgacagtgggcac C/A actctcagggaacccattac | 2121 | | |
| ABCA4 | 48 | intron4 + 3923 | gcaccactctcagggaaccc A/G ttactgtgagagaagccact | 2122 | | |
| ABCA4 | 49 | intron4 + 3952 | agagaagccactgtgccact G/C tgtggtcgaacttcaagacc | 2123 | | |
| ABCA4 | 50 | intron4 + 4125 | ggctgtccagcacacagggg C/A aggcctcttggccactgggg | 2124 | | |
| ABCA4 | 51 | intron4 + 4637 | aatcacttgccccaaggtca C/T cttaactgttaggtgttctt | 2125 | | |
| ABCA4 | 52 | intron4 + 5319 | acctctaggggctcccagag A/G ccccaagaacagaaccttcc | 2126 | | |
| ABCA4 | 53 | intron6 + 2266 | cacccttgcagacctcagac G/A ggtcctgggggcttgctttc | 2127 | | |
| ABCA4 | 54 | intron6 + 2857 | ccagaggagaaagctctgcc G/A tagtcggcctcagttaacca | 2128 | | |
| ABCA4 | 55 | intron6 + 2861 | aggagaaagctctgccgtag T/C cggcctcagttaaccacgga | 2129 | | |
| ABCA4 | 56 | intron6 + 3078 | gcaggcattaaaatgggact T/G tgcctttattgctcctgggc | 2130 | | |
| ABCA4 | 57 | intron6 + 3375 | ttaaatgccaaatgagttct C/G attaacaaagaaagagggaa | 2131 | | |
| ABCA4 | 58 | intron6 + 3412 | ggaaaatctcagtaaaccac C/T gtgacggcatctacccactt | 2132 | | |
| ABCA4 | 59 | intron6 + 4635 | ctttcgggtggatattgcta C/T gtcaagtgtctgggaaagcc | 2133 | | |
| ABCA4 | 60 | intron6 + 5576 | ccactaatatgcattcttta G/C taagcggtctcaatatacac | 2134 | | |
| ABCA4 | 61 | intron6 + 5825 | aaaaagcattttgctcttat A/G aaagcacagcctcttttgag | 2135 | | |
| ABCA4 | 62 | intron6 + 5916 | cccagacaacccaagcagag A/G cctcttagggccggaatcat | 2136 | | |
| ABCA4 | 63 | intron6 + 6993 | agcacaggatcaaggcctaa A/G ggccccttagactgacctca | 2137 | | |
| ABCA4 | 64 | intron6 + 7242 | ttgccattttgatctgtgac T/C tttttttccagaaatagttt | 2138 | | |
| ABCA4 | 65 | intron6 + 7454 | atggagggctccctcgggac T/C aggcagtattcagagatgta | 2139 | | |
| ABCA4 | 66 | intron6 − 264 | aaacagcaattagaatcact T/C tgaaatagtgatagtattta | 2140 | | |
| ABCA4 | 67 | intron6 − 86 | aggagggggggagttttcaa A/G catataggagatcagactgt | 2141 | | |
| ABCA4 | 68 | intron6 − 32 | tatacctacaaacatatata T/C atttaaaaaattgttttact | 2142 | | |
| ABCA4 | 69 | intron7 + 828 | gatgtgggaaagttagagaa G/C agcccattgtactaatgctc | 2143 | | |
| ABCA4 | 70 | intron7 + 1019 | aggcttcttgactgtctaga T/C agcaagtctaatcatttgtg | 2144 | | |
| ABCA4 | 71 | intron8 + 374 | gtaaacacggctgtgggatg C/T ttttacaaacacaatatcgt | 2145 | | |
| ABCA4 | 72 | intron8 + 874 | tgatgagcttgttattggtg G/A ggtacagcctattaatttag | 2146 | | |
| ABCA4 | 73 | intron9 + 605 | tcgtgtctctgtcttgatct C/T tgtctggttttaggccaact | 2147 | | |
| ABCA4 | 74 | intron10 + 1268 | aacttttgaagaactggaac G/A cgttaggaagttggtcaaag | 2148 | | |
| ABCA4 | 75 | intron10 + 1269 | acttttgaagaactggaacg C/T gttaggaagttggtcaaagc | 2149 | | |
| ABCA4 | 76 | intron11 + 5236 | ggcctggcacagatgaaata C/T tattcagagttcacagtgta | 2150 | | |
| ABCA4 | 77 | intron11 + 5270 | cagtgtattttcatttcata A/G tatatttgattttcaggtct | 2151 | | |
| ABCA4 | 78 | intron11 + 5687 | atcatgtaatgtactttaga C/G tcagatatataaatatttgt | 2152 | | |
| ABCA4 | 79 | intron11 + 7136 | gacttcccaacttaccttag T/C ggagctgtagtcacatagaa | 2153 | | |
| ABCA4 | 80 | intron11 + 7180 | acgctcataaatgcttctct G/A ggctgtaaaggttgaatttt | 2154 | | |
| ABCA4 | 81 | intron11 + 7701 | gttagacgcaggcattacct C/T gtggctttgccccagtgtga | 2155 | | |
| ABCA4 | 82 | intron11 + 8073 | gggatgtttgcccacatcca T/C tggcatttctcaaaaggaac | 2156 | | |
| ABCA4 | 83 | intron11 + 8586 | cagctgcctgcgctggagag G/A gctcaaacctcttccgccag | 2157 | | |
| ABCA4 | 84 | intron11 + 8893 | agcaaagatgccctttgact C/T cttttcccactagtggtgct | 2158 | | |
| ABCA4 | 85 | intron11 + 9257 | gaatgaggtcacttgctgca T/A ggcaggtggcttccccatga | 2159 | | |
| ABCA4 | 86 | intron11 + 11234 | cccaaataattttgtttttc G/A ttttaggaattaaatttcag | 2160 | | |
| ABCA4 | 87 | intron11 + 11641 | aagaaacaaacatttattga C/G aacttttggtgtgtgacctg | 2161 | | |
| ABCA4 | 88 | intron11 + 11808 | tggtatttcttaaagaaata C/T caattccatttccttttaac | 2162 | | |
| ABCA4 | 89 | intron11 + 11923 | aagatcattattaatatctc A/G tcagcgtggtgtcacttaag | 2163 | | |
| ABCA4 | 90 | intron11 + 12055 | tgagaacattacatgggacc T/C gcccccagggcatggaggct | 2164 | | |
| ABCA4 | 91 | intron11 + 305 | tcaccctgtggtcgggaggt G/A tgagtgagctatccaagccc | 2165 | | |
| ABCA4 | 92 | intron11 + 1461 | ttgggtttcagtgtcagcat G/A tagctgtctactcagatccc | 2166 | | |
| ABCA4 | 93 | intron12 + 1237 | aagggcaccaaagttctaag A/G gatgaggggaggagctgagc | 2167 | | |
| ABCA4 | 94 | intron13 + 1269 | ggagctgagccccttgtcct T/C atctaggtttcccttgttct | 2168 | | |
| ABCA4 | 95 | intron14 + 1309 | ttcccatccctcagtctgct T/C cttttcccagtaccaacatg | 2169 | | |
| ABCA4 | 96 | intron14 + 2979 | tcacctgtgtgggtagcaaa C/T ctcagaaaatcaagtataga | 2170 | | |
| ABCA4 | 97 | intron14 + 23 | gagtcctttaaaacacaaat C/G ttaatgtttgaaatcaactc | 2171 | | |
| ABCA4 | 98 | intron14 + 204 | tgctgggccctgtgtgatca T/G gaatggctgatcatggatga | 2172 | | |
| ABCA4 | 99 | intron17 + 715 | gggactcccctagagctgaa G/A tactctcccatctgtttgtt | 2173 | | |
| ABCA4 | 100 | intron17 + 1282 | ggaagatgaagaacctaagc C/T gcttccagaaattcatgagg | 2174 | | |
| ABCA4 | 101 | intron17 + 1531 | gtctaccccttaggaccatt G/A taagagtacattgaggtaat | 2175 | | |
| ABCA4 | 102 | intron18 + 1802 | actgctcacccaggaggcaa C/A gcctcgagtcatgcaccgaa | 2176 | | |
| ABCA4 | 103 | intron18 + 195 | acagattattccattgtatg C/A atgaactatgtaagccatcc | 2177 | | |
| ABCA4 | 104 | intron19 + 755 | ctggctgccgctggggtttc C/T tatgtccatccacggggagg | 2178 | | |
| ABCA4 | 105 | intron20 − 497 | ctgagttaggtctagatggg G/A acactttggatgaatgagga | 2179 | | |
| ABCA4 | 106 | intron23 + 702 | tatcaaatacaactcagacg T/G cagtctcctggcccctttga | 2180 | | |
| ABCA4 | 107 | intron26 + 156 | cctgctttccaaacccttat C/T ttgattcttggtaacatgaa | 2181 | | |
| ABCA4 | 108 | intron26 + 385 | tttaaagaacagtgagtcac G/A tgacttgctctttgaaatgc | 2182 | | |
| ABCA4 | 109 | intron27 + 299 | gacatgccatcagaccactg C/T gagtgttcaggcagcctacc | 2183 | | |
| ABCA4 | 110 | intron27 + 168 | ctccttccacacttgtgtgc A/G gggacattcactacctccta | 2184 | | |
| ABCA4 | 111 | intron28 + 497 | gctgtcaataaggaccaaaa C/T agactaatttcaaattcctc | 2185 | | |
| ABCA4 | 112 | intron29 + 587 | agctgctaggaataaaaggg G/A agacaaaacgatccacaagc | 2186 | | |
| ABCA4 | 113 | intron29 + 577 | aataaaaagggagacaaaac G/A atccacaagctagagatggt | 2187 | | |
| ABCA4 | 114 | intron29 + 2494 | aatcacagctcatctgctgc A/G tcctagggatcccaaaagaa | 2188 | | |
| ABCA4 | 115 | intron29 + 2169 | aatgtaacagccaaagtcct A/G gaaaaaggcaagccagttcc | 2189 | | |
| ABCA4 | 116 | intron30 − 535 | ctaactgtgaattatcatct T/G tgatcactgccctttgagat | 2190 | | |
| ABCA4 | 117 | intron30 − 957 | gagttctcagcagcaaatct C/A cagtatgaaattttggattt | 2191 | | |
| ABCA4 | 118 | intron31 + 445 | tccagaggtttagaacctca C/T caagtgggactctaggagcc | 2192 | | |
| ABCA4 | 119 | intron31 + 48 | aggatttttgacttgcttaa C/T taccatgaatgagaaactct | 2193 | | |
| ABCA4 | 120 | intron32 + 129 | tgtttagtcaggcacatatg A/C acatccgactttcaaataag | 2194 | | |
| ABCA4 | 121 | intron33 + 209 | tctccccaacatttatgtgg C/A aagtaagtttacatttggtt | 2195 | | |
| ABCA4 | 122 | intron35 + 3209 | ttgaggcctccacaccccac G/A gcaggttgccccctgaggaa | 2196 | | |
| ABCA4 | 123 | intron35 + 3542 | cttggcagggaggtagggca T/C ggggtggggtaggaggacta | 2197 | | |
| ABCA4 | 124 | intron36 + 304 | ctggggcagccattccccca A/G cccctcacccagctctgact | 2198 | | |
| ABCA4 | 125 | intron36 + 525 | taaatttgaatgagtaatta A/G tccatctcggcctcagtttc | 2199 | | |
| ABCA4 | 126 | intron37 + 766 | tgttgcaggctggagaaccc T/G cctatgaattgtacagggct | 2200 | | |
| ABCA4 | 127 | intron37 + 856 | aaaaccccatgaagtggtca A/G ggcaggcatcattatctcca | 2201 | | |
| ABCA4 | 128 | intron37 + 62 | tagtagagtatgtgttggtc G/A agcagagccaggggcaagca | 2202 | | |
| ABCA4 | 129 | intron37 + 761 | tccttgggcaagttaatctt G/A atgaagagactgggtgttct | 2203 | | |
| ABCA4 | 130 | intron38 + 1315 | cagagtcagactctggaaag G/T cggggggataagaacacagc | 2204 | | |
| ABCA4 | 131 | intron38 + 1316 | agagtcagactctggaaagg C/A ggggggataagaacacagcc | 2205 | | |
| ABCA4 | 132 | intron38 + 1525 | ccaacatttgctaagcaccc G/A ccttcaaaaacctggtattt | 2206 | | |
| ABCA4 | 133 | intron38 + 1561 | gtattttcatgtaaattatc C/A gatacacagctgctatggaa | 2207 | | |
| ABCA4 | 134 | intron38 + 1562 | tattttcatgtaaattatcc G/A atacacagctgctatggaaa | 2208 | | |
| ABCA4 | 135 | intron38 + 1674 | ccagctgaacaccacgtgcc G/A ggtgtgtgctgatataaaca | 2209 | | |
| ABCA4 | 136 | intron38 + 2867 | tgcctggctagacaaagggg A/C agctcccgcccactagaaac | 2210 | | |
| ABCA4 | 137 | intron38 + 2874 | ctagacaaaggggaagctcc C/T gcccactagaaacttgcagg | 2211 | | |
| ABCA4 | 138 | intron38 + 123 | gaggggaccttgttgggctg G/A aggtgtcctgccagctggag | 2212 | | |
| ABCA4 | 139 | intron40 + 1904 | gacactgtacagccagccca A/C tcctgaccccttttcttcat | 2213 | | |
| ABCA4 | 140 | exon41 + 5814 | ggaaataaaactgacatctt A/G aggctacatgaactaaccaa | 2214 | | |
| ABCA4 | 141 | intron41 + 122 | atttggttcccagttttatg T/G agggtcatcatccctgtgtt | 2215 | | |
| ABCA4 | 142 | intron41 + 287 | tctgcagagcatgggtcagc C/T tcgagatgtctcagtactca | 2216 | | |
| ABCA4 | 143 | intron41 + 411 | cctcttcccctccttgctct C/A accctgtctcagttctcagt | 2217 | | |
| ABCA4 | 144 | intron41 + 443 | gttctcagtccggtttcttc G/A tatcttgcagatttatccag | 2218 | | |
| ABCA4 | 145 | exon42 + 5844 | cgtatcttgcagatttatcc A/G ggcacctccagcccagcagt | 2219 | | |
| ABCA4 | 146 | intro43 + 328 | tttgtagcctattcctataa A/G aatgcaccattgcttcccat | 2220 | | |
| ABCA4 | 147 | intron43 + 345 | taaaaatgcaccattgcttc C/G cattacctccctccacacat | 2221 | | |
| ABCA4 | 148 | intron43 + 370 | acctccctccacacattttt A/G caaaacgtttcagggagttt | 2222 | | |
| ABCA4 | 149 | intron43 + 376 | ctccacacatttttacaaaa C/T gtttcagggagtttactgag | 2223 | | |
| ABCA4 | 150 | intron43 + 670 | ttaaacagactggtccccta T/C gggcaggacagagaggatga | 2224 | | |
| ABCA4 | 151 | intron43 + 701 | gagaggatgagctctcactc A/G tctgcctctttcctggctgc | 2225 | | |
| ABCA4 | 152 | intron43 + 822 | gttaggtgctgctgacatct G/A tccagcatctgcttgactgg | 2226 | | |
| ABCA4 | 153 | intron43 + 915 | ggcaggacgagtcctgagca C/T gcttcactggctcagacagg | 2227 | | |
| ABCA4 | 154 | intron43 + 1242 | actgagctggacgctagaaa G/T aaactataggcttaagacac | 2228 | | |
| ABCA4 | 155 | intron43 + 1671 | tagagaagtttacttccatc G/A ggacacatgcatcttttcta | 2229 | | |
| ABCA4 | 156 | intron43 + 2036 | ttgaaggatactcagtaatt G/A ctttttttcttgcagtattt | 2230 | | |
| ABCA4 | 157 | intron45 + 176 | gtgtttggttcacacagctc C/T ggagaaaaacaagtcacggc | 2231 | | |
| ABCA4 | 158 | intron45 + 193 | ctccggagaaaaacaagtca C/T ggcacagccttgacttggga | 2232 | | |
| ABCA4 | 159 | intron47 + 238 | cccaagtctctggatggggc A/G tctgatcaggatgcatgcag | 2233 | | |
| ABCA4 | 160 | intron47 + 269 | atgcatgcagagcctggctg G/A gatgagggagggctgctacc | 2234 | | |
| ABCA4 | 161 | intron47 + 326 | accacttatctcaacagatc C/G gggacctgtggcctatttac | 2235 | | |
| ABCA4 | 162 | intron47 + 715 | aagtcactaagctggttggt G/A ggaggaacagcacataaccc | 2236 | | |
| ABCA4 | 163 | intron47 + 734 | tgggaggaacagcacataac C/T caccttatctatgctgaggt | 2237 | | |
| ABCA4 | 164 | intron47 + 931 | ggacactgcatagatatcta T/C agaaatagcagcatgtcagg | 2238 | | |
| ABCA4 | 165 | intron47 + 1260 | acactctctggtggaccatc A/C ctcatccaagagagggtaac | 2239 | | |
| ABCA4 | 166 | intron48 + 1663 | tctcgctcttctcttacctc T/C aggtgtttgtaaattttgct | 2240 | | |
| ABCA4 | 167 | intron49 + 127 | agagagccccacccacacca C/T ggtccctaccaagtccccac | 2241 | | |
| ABCA4 | 168 | intron49 − 1545 | gcagttaattccaaactttt C/A tcccttattggatgagatca | 2242 | | |
| ABCA4 | 169 | 5′flanking − (1441-1400) | gtaaatctcagttgaatcag (TCA)14-15 atttttcagtctggttcctg | 2243 | | |
| ABCA4 | 170 | intron1 + (4712-4720) | gaggggcggggactataggc (A)8-10 cagcctaattcaaggatgag | 2244 | | |
| ABCA4 | 171 | intron2 + 7295-7304) | ttgttggctttgcaatggat CACAGTCAT/Δ ttattcactc | 2245 | | |
| | | | attcattcac | | | |
| ABCA4 | 172 | intron3 + (951-952) | cctgtccatcagactcttct TT/Δ acctctccccgaggagccca | 2246 | | |
| ABCA4 | 173 | intron4 + (2642-2653) | cctgggtgacagagcgagat (A)10-12 tagcatgagatattattact | 2247 | | |
| ABCA4 | 174 | intron6 + 5202 | cacaaagcatctgacacccc C/Δ agactaggagaaggtctgtc | 2248 | | |
| ABCA4 | 175 | intron6 + (3029-3044) | cactaaaaacaaaaatttac (A)16-18 cttttatggatttacaaaga | 2249 | | |
| ABCA4 | 176 | intron6 + (5138-5139) | ttcatgacagatcagatgtt (G) cttttatggatttacaaaga | 2250 | | |
| ABCA4 | 176 | intron6 + (5138-5139) | ttcatgacagctcagatgtt cttttatggatttacaaaga | 2251 | | |
| ABCA4 | 177 | intron6 + 5985 | tttccttcttcaaacccccc C/Δ agactaggagaaggtctgtc | 2252 | | |
| ABCA4 | 178 | intron6 + 6094 | gggacggacagaaaaagacc T/Δ agtttctgttgagccaaaga | 2253 | | |
| ABCA4 | 179 | intron6 − 161 | tattttttcaattaaataaa A/Δ gagttttttgtttctaaaag | 2254 | | |
| ABCA4 | 180 | intron7 + (809-810) | gggccgagtatgcacactga (TG) tgtgggaaagttagagaaga | 2255 | | |
| ABCA4 | 180 | intron7 + (809-810) | gggccgagtatgcacactga tgtgggaaagttagagaaga | 2256 | | |
| ABCA4 | 181 | intron8 + (472-484) | atcttccccacctttcacta (T)10-13 ggtcttctatggggtaaagg | 2257 | | |
| ABCA4 | 182 | intron9 + (48-71) | gtaccctggacctcccagaa (GT)11-13 gagagagatgtgccttcctg | 2258 | | |
| ABCA4 | 183 | intron9 + 554 | ataggggcagaaaagacaca A/Δ ccaaaagttctctctcactt | 2259 | | |
| ABCA4 | 184 | intron10 + 11 | catgatcagagtaagggggg G/Δ ttggaggatggggaggggcg | 2260 | | |
| ABCA4 | 185 | intron11 + 4242 | ggagaggaaatgatgttagt G/Δ cctcctgtaaataggcccag | 2261 | | |
| ABCA4 | 186 | intron11 + (13743-13753) | tgctcttttgtgggtaatgg (T)9-11 cctcttccaggagaagaaaa | 2262 | | |
| ABCA4 | 187 | intron13 + (636-637) | cggggtggagggttgggagg (G) ctcatttgtcattatagatg | 2263 | | |
| ABCA4 | 187 | intron13 + (636-637) | cggggtggagggttgggagg ctcatttgtcattatagatg | 2264 | | |
| ABCA4 | 188 | intron18 + (569-570) | tgctgccctcatcttctctc TT/Δ aaactagttctgtatttctc | 2265 | | |
| ABCA4 | 189 | intron20 − (304-297) | tataacctgacttttttttc (A)7-9 ggattgcttttttaaacata | 2266 | | |
| ABCA4 | 190 | intron22 + (1236-1246) | gctgaattagttcccttggg (T)9-11 agttaactcctgatttttgc | 2267 | | |
| ABCA4 | 191 | intron26 + (4626-4635) | gataatcaatgctgtaaggg (A)9-10 tggcattagagatccagacc | 2268 | | |
| ABCA4 | 192 | intron33 + (115-116) | taaaaccgtcttgtttgttt GT/Δ ttacatggtttttagggccc | 2269 | | |
| ABCA4 | 193 | intron36 + 1078 | taagcagctatcacttaaca (T) cagccattccccaacccctc | 2270 | | |
| ABCA4 | 194 | intron37 + (290-291) | ccttgaccaaagcctggggg (T) cagccattccccaacccctc | 2271 | | |
| ABCA4 | 194 | intron37 + (290-291) | ccttgaccaaagcctggggg cagccattccccaacccctc | 2272 | | |
| ABCA4 | 195 | intron38 + 986 | ataaaaagaggggggaaaaa A/Δ gaagacagtcgctgcagggc | 2273 | | |
| ABCA4 | 196 | intron38 + (1206-1210) | gtggaccaactgagactgact CT/Δ ttccagatcttgttagggtt | 2274 | | |
| ABCA4 | 197 | intron38 + 1322 | actctggaaaaggcgggggg G/Δ ataagaaaacagccccagca | 2275 | | |
| ABCA4 | 198 | intron38 + 3107 | gggccccatctgctgaagag A/Δ ggggggtggggtttgccccc | 2276 | | |
| ABCA4 | 199 | intron40 + 182 | ttttctccaataatacaagt A/Δ gaggatcgggttaaaatagg | 2277 | | |
| ABCA4 | 200 | intron43 + 330 | tttaattggcacagccatgc A/Δ tgcaccattgcttccgatta | 2278 | | |
| ABCA4 | 201 | intron43 + 1354 | tttaattggcacagccatgc C/Δ tttggtggcttttgtcattg | 2279 | | |
| ABCA4 | 202 | intron47 + (1305-1306) | gatcatgctgaaggagaaag AAAG/Δ caccaatggcccaagcccta | 2280 | | |
| ABCA7 | 1 | 5′flanking − 1588 | agaatgttggccccctccgg C/T tcctgcatggtctgcagaag | 2281 | | |
| ABCA7 | 2 | 5′flanking − 1584 | aatgttgggcccctccccgt C/T ctgcatcctctgcagaagcc | 2282 | | |
| ABCA7 | 3 | 5′flanking − 1180 | ggccagtgagtgacgggcag G/A tcgccaaaatagcagcgtga | 2283 | | |
| ABCA7 | 4 | 5′flanking − 480 | agagctggggtcgtgcctcc A/G gctgggcaactgcctgtctc | 2284 | | |
| ABCA7 | 5 | 5′untranslated − 9 | gtgtgtcacgtcccctgccg A/G gtctcaccatggccttctgg | 2285 | | |
| ABCA7 | 6 | intron5 + 81 | ccccgggccaaggacatccc G/A ttccaggcatccagtctgtc | 2286 | | |
| ABCA7 | 7 | exon6 + 583 | cagcttgttggaggccgctg A/G ggacctggcccaggaggtac | 2287 | | |
| ABCA7 | 8 | intron6 + 183 | gccggagggtcacggaaact A/G tttgaagaagtaggagttag | 2288 | | |
| ABCA7 | 9 | intron8 + 164 | tgtggaggatcagaggcaca C/T gcaggagcaaggcagagggg | 2289 | | |
| ABCA7 | 10 | exon8 + 955 | accggaccttagaggagcta A/G ccctgctgagggatgtccgg | 2290 | | |
| ABCA7 | 11 | intron9 + 421 | tttttttttttttttttttt T/A taagagatggagtctgactc | 2291 | | |
| ABCA7 | 12 | intron9 + 453 | gttgcccaggctggactgca G/A tggcgagatcttggctcact | 2292 | | |
| ABCA7 | 13 | intron9 + 467 | cccaggctggactgcagtgg C/T gagatcttggctcactgcaa | 2293 | | |
| ABCA7 | 14 | intron9 + 488 | gagatcttggctgactgaaa C/T ctccgcctcgtggattcaag | 2294 | | |
| ABCA7 | 15 | exon10 + 1184 | cgcacacgctgatgtgggaa A/G actggtgggcacgctgggcc | 2295 | | |
| ABCA7 | 16 | intron10 + 70 | gagtgacggaggtgagggac T/C gtccacctgcggggtctgtt | 2296 | | |
| ABCA7 | 17 | exon11 + 1306 | cctgggccccggccacgtgc G/A catcaaaatcagcatggaaa | 2297 | | |
| ABCA7 | 18 | intron12 + 118 | cccttcctgctgagcgccgg T/G ttacaaaaatccacgtgacc | 2298 | | |
| ABCA7 | 19 | exon13 + 1824 | accttcctgctgagcgacgc A/C ctgctggttatggtgctcaa | 2299 | | |
| ABCA7 | 20 | intron13 + 56 | ggtgcgctggagggtgacag A/G gaggggcggcaccacgtcgg | 2300 | | |
| ABCA7 | 21 | intron13 + 78 | ggggcggccccacgtgggtg C/A gcgccaccaggccaatccag | 2301 | | |
| ABCA7 | 22 | exon14 + 1851 | cgttgcctctcacagctggg A/G gacatactcccatacagcca | 2302 | | |
| ABCA7 | 23 | exon16 + 2153 | cgagggcgcgcagtggaaca A/C agtgggcacccggactacgg | 2303 | | |
| ABCA7 | 24 | intron16 + 34 | ggcggggctccgggccgggt C/G gcaaatgctttgcgggaggc | 2304 | | |
| ABCA7 | 25 | intron16 + 8 | ctggacccaaagggtgaggc A/C ctacgaggcttaatagctgg | 2305 | | |
| ABCA7 | 26 | intron16 + 161 | tcccgcagcttttataggcc C/T aggcccagcaggtaacggat | 2306 | | |
| ABCA7 | 27 | exon17 + 2386 | caccccatctctgcagtgct G/A gtagaagaggcacgggccgg | 2307 | | |
| ABCA7 | 28 | exon17 + 3427 | accggcctgagtcctggcgt C/A tccgttcgcagcctggagaa | 2308 | | |
| ABCA7 | 29 | intron20 + 168 | cgagacagtaagagttgggg A/G tagacagaggttcccctgga | 2309 | | |
| ABCA7 | 30 | exon21 + 3027 | ctgctgggagaccgtgtggc C/T gtgatggcaggtggccgatt | 2310 | | |
| ABCA7 | 31 | intron22 + 1386 | gggtggggcgtgagccgggg C/T tacctgaagcaaccctttgt | 2311 | | |
| ABCA7 | 32 | exon23 + 3417 | gggatctccgacaacagcct G/G gaggaggtgtgaggcgtggg | 2312 | | |
| ABCA7 | 33 | intron23 + 147 | ggagctctggtgactaagat G/A taactcgggaaggcctgggg | 2313 | | |
| ABCA7 | 34 | intron26 + 3628 | gctggcctagacgtaaccat A/G cggctaaagatgccgcaaca | 2314 | | |
| ABCA7 | 35 | exon29 + 4046 | cccagcctgccagtgtagcc G/A gcccggtgccaggcgcctgc | 2315 | | |
| ABCA7 | 36 | intron30 + 81 | ccccctgggagctataccgg C/A ccccccggctctcagctcac | 2316 | | |
| ABCA7 | 37 | exon31 + 4238 | ctgcctgcatggccccacag A/C tacggaggcttctcgctggg | 2317 | | |
| ABCA7 | 38 | intron32 + 1 | caaggagcagctgtctgagg G/C tgcactgtgagtccctccac | 2318 | | |
| ABCA7 | 39 | intron33 + 54 | ccactgcttgacactgcaat G/A tatggcaccttgtaggcagg | 2319 | | |
| ABCA7 | 40 | intron34 + 245 | cagtactttgggaggccgag G/A caggaggactgattgtggcc | 2320 | | |
| ABCA7 | 41 | exon36 + 3057 | ggtgagcggatcttgaaaaa A/G ggtcttccttatgttccgag | 2321 | | |
| ABCA7 | 42 | intron38 + 66 | ggcccactcacctttgtgca A/G gaactgcaatctcccaggta | 2322 | | |
| ABCA7 | 43 | intron40 + 154 | ctctaactccacacacaaaa C/G caggctctgagacaaacctg | 2323 | | |
| ABCA7 | 44 | intron40 + 277 | ctgagcccccggagccacca T/C aaccagcgtggcccgggaac | 2234 | | |
| ABCA7 | 45 | intron41 + 3392 | gtggcccgggaacccagtgc T/C gagcacctgagcatgggata | 2325 | | |
| ABCA7 | 46 | intron41 + 286 | ctccttgactctgagttctg T/C ggccgtgcacacttgctcat | 2326 | | |
| ABCA7 | 47 | intron41 + 396 | tggcagttcccagtttgcag C/T cgtttcactgcctcttcaat | 2327 | | |
| ABCA7 | 48 | intron41 + 391 | cacactatggccctgcccca C/T accatcccagctccacccaa | 2328 | | |
| ABCA7 | 49 | intron41 + 394 | actatggccctgccgcagac C/T catcccagctggacacacaa | 2329 | | |
| ABCA7 | 50 | intron41 + 398 | tggccctgccccacacccat C/G acagctcgacccacaccatg | 2330 | | |
| ABCA7 | 51 | intron41 + 1001 | ccctgccccacacccatagc A/G gctccacccacaccatgggg | 2331 | | |
| ABCA7 | 52 | intron41 + 1051 | actcatgctggctccaccca C/T accatggccccgccccatac | 2332 | | |
| ABCA7 | 53 | intron41 + 1131 | tgccctgccccatgcccatt A/G tgcccctgctccacactcaa | 2333 | | |
| ABCA7 | 54 | exon44 + 5985 | gaagcgctctgctcgcgcct G/A gccatcatggtgaatgggcg | 2334 | | |
| ABCA7 | 55 | intron44 + 201 | ggcgcaggaccaggaggcgt G/C agccgggggctctgggtgga | 2335 | | |
| ABCA7 | 56 | intron44 + 233 | ctgggtggatttagaagaca C/T aatcaggtgtgcgttggagt | 2336 | | |
| ABCA7 | 57 | intron44 + 313 | agttaggggagggcctggtt A/G gtgggcggggccataggaaa | 2337 | | |
| ABCA7 | 58 | intron44 + 337 | ggcggggccataggaaagtg G/T cggagctgcgcgaggcacat | 2338 | | |
| ABCA7 | 59 | exon45 + 6133 | tggcggccgagttccctggg G/T cggagctgcgcgaggcacat | 2339 | | |
| ABCA7 | 60 | exon45 + 6159 | ctgcgcgaggcacatggagg C/T cgcctgcgcttccagctgcc | 2340 | | |
| ABCA7 | 61 | intron45 + 27 | acggcgccggggtcgggctg G/C gggaggcaggctgggggcca | 2341 | | |
| ABCA7 | 62 | 3′untranslated + 6580 | aaggctggagagaagccgtg G/C tggtgaaaccgtgtgcatgt | 2342 | | |
| ABCA7 | 63 | 3′flanking + 108 | caagctgagtgtgcacatac G/A ggccaagtggcgattcatag | 2343 | | |
| ABCA7 | 64 | 3′flanking + 376 | cttacaggagcccggtgtcc C/T ggagcacaggccagggccgg | 2344 | | |
| ABCA7 | 65 | 3′flanking + 687 | cagcagggagacttggggag G/A ggggagagagttcacactgc | 2345 | | |
| ABCA7 | 66 | 3′flanking + 688 | agcagggagacttggggagg G/A gggagagagttcacactgcg | 2346 | | |
| ABCA7 | 67 | 3′flanking + 1169 | cctcgacctgacccacttca C/T ggggctgcagggcgggtgat | 2347 | | |
| ABCA7 | 68 | intron9 + (398-422) | tgtgaactaccacgtcctgc (T)22-26 aagagatggagtctcactct | 2348 | | |
| ABCA7 | 69 | intro121 + (175-184) | ggggactctgagggtctggt (G)8-10 actctgagggtctgggggcc | 2349 | | |
| ABCA7 | 70 | intron30 + (81-87) | ccccctgggagctctcccgg (C)6-7 ggccctcagctccccttccc | 2350 | | |
| ABCA7 | 71 | intron34 + (349-361) | agaaagagaaagagagaaag (A)12-14 cagaaatgtgctttgggtga | 2351 | | |
| ABCG1 | 1 | 5′flanking − 1772 | cctgggcttcagcaggggcc T/C cacacctgcaatgggtgcct | 2352 | | |
| ABCG1 | 2 | 5′flanking − 1754 | cctcacacctgcaatgggtg C/T ctggggagagggtgcagatg | 2353 | | |
| ABCG1 | 3 | 5′flanking − 1450 | tccaaagcccagatttggtg T/C ttttggggctcttttggaat | 2354 | | |
| ABCG1 | 4 | intron1 + 4 | ctggtggaggaagaaaggta G/A ggagggcggctgctttgtgt | 2355 | | |
| ABCG1 | 5 | intron1 + 576 | agctcaggaggtgtctggaa C/T gccacacagtgcaggagttt | 2356 | | |
| ABCG1 | 6 | intron1 + 1426 | aattctccttctaaacttaa A/G gaaatattttatagaaaaat | 2357 | | |
| ABCG1 | 7 | intron1 + 2342 | agagcctgcaatgggccgcc G/A agggacctgcccatgactca | 2358 | | |
| ABCG1 | 8 | intron1 + 2399 | gaggggttgacagacaggat A/G tgtctgctgtgttccagctg | 2359 | | |
| ABCG1 | 9 | intron1 + 2406 | tgacagacaggatatgtctg C/G tgtgttccagctgctggttt | 2360 | | |
| ABCG1 | 10 | intron1 + 2911 | ccctctctgtgcccactgtt G/C tcccaacaccagcctgttct | 2361 | | |
| ABCG1 | 11 | intron1 + 4363 | tataatagattcctagcaga A/G aacataattgtgagaggaac | 2362 | | |
| ABCG1 | 12 | intron1 + 4752 | gctttcagagcccattcaca C/T aagggtctcattttattagg | 2363 | | |
| ABCG1 | 13 | intron1 + 5026 | ccaggtctgtgggatttcag G/A ccaaaaaggagcgtagcaag | 2364 | | |
| ABCG1 | 14 | intron1 + 5532 | gggttaaatattccgggcag C/T gccaagtcagattatctgta | 2365 | | |
| ABCG1 | 15 | intron1 + 5681 | gctaaagtgcatggaaggca T/C catgaataaatcctttcagg | 2366 | | |
| ABCG1 | 16 | intron1 + 6290 | tcacagcagattcatgagag T/A tgaatgtttagccgccatgt | 2367 | | |
| ABCG1 | 17 | intron1 + 6386 | agatgctcccctccagccag C/T acattttctccctgtgagca | 2368 | | |
| ABCG1 | 18 | intron1 + 6758 | acctgcatggtgggtgcccc C/G ctgccttcctctactgcctt | 2369 | | |
| ABCG1 | 19 | intron1 + 7029 | tgggtcagattaaatatatc C/T tgaaggactaaaccgtaaaa | 2370 | | |
| ABCG1 | 20 | intron1 + 7176 | ttgctcacattgtgaaaaaa C/G gcaaaaagatgggttttcag | 2371 | | |
| ABCG1 | 21 | intron1 + 8243 | gcctgagagcgctggcagta G/A gaagggtcgccagtgtggac | 2372 | | |
| ABCG1 | 22 | intron1 + 11224 | tctggtttagagaggaaaat G/A ggcagcatcattttgtcacc | 2373 | | |
| ABCG1 | 23 | intron1 + 11371 | gggctctcttggagcccttt T/G tctctcccagccctgcgtct | 2374 | | |
| ABCG1 | 24 | intron1 + 12420 | gagttgtcctccaagagaat C/T tttgtatggttccttttctg | 2375 | | |
| ABCG1 | 25 | intron1 + 12484 | gagttgtcctccaagagaat G/T tttgtatggttccttttctg | 2376 | | |
| ABCG1 | 26 | intron1 + 12955 | ctggggttggtgggagccac A/G gtctcacacctattggcagg | 2377 | | |
| ABCG1 | 27 | intron1 + 12985 | ctcttggcaggtcgtgaaca T/C tgttcttggatttgcaaata | 2378 | | |
| ABCG1 | 28 | intron1 + 20041 | acatggccggcttcccttct T/C cctcggaatggcctggaatt | 2379 | | |
| ABCG1 | 29 | intron1 + 20046 | gccggcttcccttcttcctc G/A gaatggcctggaattcgatc | 2380 | | |
| ABCG1 | 30 | intron1 + 21058 | acaagacttagaatttgacc G/A tgattttaaaactattctaa | 2381 | | |
| ABCG1 | 31 | intron1 + 26189 | ttcttggatgtggccatgca C/T gggggcaagggtttgatgag | 2382 | | |
| ABCG1 | 32 | intron1 + 27453 | atcatgtggtttgggggaaa G/C ctgggaccccacttggtaca | 2383 | | |
| ABCG1 | 33 | intron1 + 28098 | caggaaggagacagctgctg G/C tgctgcttagagttaggcgc | 2384 | | |
| ABCG1 | 34 | intron1 + 29670 | ccttcagttgtaataggcag A/G aggagcgcacgaggaggctg | 2385 | | |
| ABCG1 | 35 | intron1 + 29810 | attgtttctcctggttttgt T/C tgtgttgactttccctttaa | 2386 | | |
| ABCG1 | 36 | intron1 + 36220 | cagatcccttggttgctggg C/T aggtagtaggagaggttttt | 2387 | | |
| ABCG1 | 37 | intron1 + 36341 | aaacagggcttgagtcctcc G/A taagggacaggagaccttcc | 2388 | | |
| ABCG1 | 38 | intron1 + 36370 | aggagaccttcccacatcct G/A gcaagaattcttcttttttc | 2389 | | |
| ABCG1 | 39 | intron1 + 36662 | cagactaaatgcacaattct G/A gattgagctgactgtattga | 2390 | | |
| ABCG1 | 40 | intron1 + 36914 | tgtaaaagatggagaagaac A/G cagtagtcgcttgctgtgag | 2391 | | |
| ABCG1 | 41 | intron1 + 37029 | tgtgactcatggcctctgcc A/G ggggactgggctggccctgc | 2392 | 2392 | |
| ABCG1 | 42 | intron4 + 1196 | tgaaaagaaaatggatgagt G/A gaaaccaaaagagagaaaat | 2393 | | |
| ABCG1 | 43 | intron4 + 1200 | aagaaaatggatgagtggaa A/C ccaaaagagagaaaatgtgg | 2394 | | |
| ABCG1 | 44 | intron4 + 2041 | aagcagaggcttttccaccc G/A gagactcaagaagctgctcc | 2395 | | |
| ABCG1 | 45 | intron4 + 2490 | gtggtgaagtagagctgagc A/T cacgggggagccctccatcc | 2396 | | |
| ABCG1 | 46 | intron4 + 2552 | atggccttgggccactgcct G/A ctgtgccccgagccgagctt | 2397 | | |
| ABCG1 | 47 | intron4 + 2822 | cagcaggctccgtgctgaag T/C cacagcaagccaggcccttg | 2398 | | |
| ABCG1 | 48 | intron4 + 2850 | agccaggcccttggcctgcc G/A gagctggaagacccagaaca | 2399 | | |
| ABCG1 | 49 | intron4 + 2919 | gcctcccaggagtagctaca C/T gggacccgaaggcagatggc | 2400 | | |
| ABCG1 | 50 | intron4 + 3506 | ggcagcctgggctgccgaga T/C cacagcaagccaggcccttg | 2401 | | |
| ABCG1 | 51 | intron4 + 3538 | cgcccgccgggaagccccag G/A ggggctggagctacaagtgg | 2402 | | |
| ABCG1 | 52 | intron4 + 3554 | ccaggggggctggagctaca A/G gtggccttgcaggttttttg | 2403 | | |
| ABCG1 | 53 | intron4 + 3721 | ccagctcatgggcaggggtg C/T ggagggaaaggcacccacag | 2404 | | |
| ABCG1 | 54 | intron4 + 3852 | caccagagccactcagtcgg C/T caagagcgtcgcccagtggt | 2405 | | |
| ABCG1 | 55 | intron4 + 3921 | gaagaccagcagtcgatgcc A/G gctgggaagagggctctgcc | 2406 | | |
| ABCG1 | 56 | intron4 + 3979 | acccaccagccttttccaga C/T agccttccagaagctgtttc | 2407 | | |
| ABCG1 | 57 | intron4 + 4291 | gagccgctggagtagggtcc G/A cttgctatggctcccagggg | 2408 | | |
| ABCG1 | 58 | intron4 + 4922 | gaaaccaccagaaattgtgc A/G tcctctcatgtgtccattca | 2409 | | |
| ABCG1 | 59 | intron4 + 4968 | tattgactggacaccttctc C/T gtatggggcactgggctagg | 2410 | | |
| ABCG1 | 60 | intron7 + 672 | atcagtaacgggtcactaac G/A gatgctgctgagtggggcag | 2411 | | |
| ABCG1 | 61 | intron7 + 840 | atttcatttcctcaatgtcg T/C ctgaccagagagcgggaggt | 2412 | | |
| ABCG1 | 62 | intron7 + 891 | tggcccactgttgagggtgt G/A ggtgaccagaggggcctgga | 2413 | | |
| ABCG1 | 63 | intron7 + 997 | tgtgtcctggtttgtggctt C/G atctaggaggtgtggtggcc | 2414 | | |
| ABCG1 | 64 | intron9 + 1616 | ctggaggagaagacaggata A/C agtctaagacgtgctgtcac | 2415 | | |
| ABCG1 | 65 | intron9 + 1630 | aggataaagtctaagacgtg C/T tgtcacagagttcagggtcc | 2416 | | |
| ABCG1 | 66 | intron9 + 1674 | gcttccaaaggccgcatccg G/T gttgttctctgagccgagga | 2417 | | |
| ABCG1 | 67 | intron9 + 1689 | gcttccaaaggccgcatccg C/T gaggacggctttgcgaacgc | 2418 | | |
| ABCG1 | 68 | intron10 + 446 | tggctgacagtgaacacagc G/A gctgcttctccagaacttta | 2419 | | |
| ABCG1 | 69 | intron10 + 581 | atgcagagtttcagaagagg C/G agactcaggaagagtaaggc | 2420 | | |
| ABCG1 | 70 | intron13 + 243 | acccggagagccatggcagg A/C ccaagtgttctggacgttgc | 2421 | | |
| ABCG1 | 71 | 3′untranslated + 2370 | gcctctcagctgatggctgc A/G cagtcagatgtctggtggca | 2422 | | |
| ABCG1 | 72 | 3′flanking + 1124 | ctcagaactacatcgagtga G/A gtcagtgttgaaaacgccca | 2423 | | |
| ABCG1 | 73 | 3′flanking + 1252 | atggggcccacagccctgcc T/C cagaagcagctttggtctcg | 2424 | | |
| ABCG1 | 74 | 3′flanking + 1433 | gggggaagagcttgggaacc A/G tgagggctgttaggctgcaa | 2425 | | |
| ABCG1 | 75 | 3′flanking + 1513 | tgaagggtgaactggagtag G/C tgaggattctgcagttgacg | 2426 | | |
| ABCG1 | 76 | intron1 + (19909-19944) | ccgatgaggaggggatgggg (CACCAGGCAGCAGACTCTGA | 2427 | | |
| | | | TGAGGAGGGGAGGGGG)caccaggcagcagactctga | | | |
| ABCG1 | 77 | intron1 + (19909-19944) | ccgatgaggagggggatgggg ca | 2428 | | |
| | | | ccaggcaagcagactctga | | | |
| ABCG1 | 78 | intron1 + (25136-25137) | catgaacttgcctgaccata (G) ccctgtgaggagctagggct | 2429 | | |
| ABCG1 | 79 | intron1 + (25136-25137) | catgaacttgcctgaccata ccctgtgaggagctagggct | 2430 | | |
| ABCG2 | 1 | intron1 + 152 | tcatttgaaagtgggtatgc G/A gtttaaaactgacagttcaa | 2431 | | |
| ABCG2 | 2 | intron1 + 614 | agctagtcataaataaatac G/A ccagagtagtaaggaagaga | 2432 | | |
| ABCG2 | 3 | intron1 + 10002 | cctcatgaatggtatacatg T/A cccaacatatctctttcgat | 2433 | | |
| ABCG2 | 4 | intron1 + 10123 | acagtggtccctttgggtgc G/A tatacccaaatccctgcata | 2434 | | |
| ABCG2 | 5 | intron1 + 10768 | ataggaataattgagaacag G/A gtctgaagaactctgcagga | 2435 | | |
| ABCG2 | 6 | intron1 + 10791 | ctgaagaactctgcaggaaa T/C gaaaatagttccctgctttt | 2436 | | |
| ABCG2 | 7 | intron1 + 10792 | tgaagaactctgcaggaaat G/A aaaatagttccctgatttta | 2437 | | |
| ABCG2 | 8 | intron1 + 14183 | tcacttaaggctttgcaggg T/G gtctaggacacagaaagaga | 2438 | | |
| ABCG2 | 9 | intron1 + 14934 | aaagtgtctttaaaatttcc A/G tcttgagtcagtgagctatt | 2439 | | |
| ABCG2 | 10 | intron1 + 14955 | tcttgagtcagtgagctatt G/T aaattcaagcaataagttat | 2440 | | |
| ABCG2 | 11 | intron1 + 17251 | ctgtttgggaacagcaactc A/C atcataggcagagagaaagt | 2441 | | |
| ABCG2 | 12 | intron1 + 17347 | atttcaaacctgtttcacaa G/A ttgttaagctcatcttaagg | 2442 | | |
| ABCG2 | 13 | intron1 + 17626 | gaaggtgcataacaacttcc T/G acataaagtctggagctata | 2443 | | |
| ABCG2 | 14 | intron1 + 18271 | aaatgaagctgctcattgcc A/G cacatttaaaaatggacttg | 2444 | | |
| ABCG2 | 15 | intron1 + 18369 | ctattgcttttctgtctgca G/T aaagataaaaactctccaga | 2445 | | |
| ABCG2 | 16 | exon2 + 34 | atgtcgaagtttttatccca G/A tgtcacaaggaaacaccaat | 2446 | | |
| ABCG2 | 17 | intron2 + 36 | tgtaaaaagacagcttttta A/G tttacctacagtgaacctca | 2447 | | |
| ABCG2 | 18 | intron2 + 4230 | caaccctaaattggagggcc C/T gggcgtggtgattgagaaag | 2448 | | |
| ABCG2 | 19 | intron2 + 4518 | gttgacagacttttatagtg A/C gggacactgacctgcatgca | 2449 | | |
| ABCG2 | 20 | intron2 + 6278 | atgtatgtaccacgtcttca T/C attcttaaaggatgacccta | 2450 | | |
| ABCG2 | 21 | intron3 + 10 | ggcaaatcttcgtgagtata A/G gagagtataagtaagcgttt | 2451 | | |
| ABCG2 | 22 | intron5 + 421 | tgccggtgagagaaaactta C/A agttctcagcagctcttcgg | 2452 | | |
| ABCG2 | 23 | intron6 + 3158 | actattctagttgattctag A/G ttgtcaatacaacacactga | 2453 | | |
| ABCG2 | 24 | intron6 + 3203 | tcctattctgttttaataaa A/G gcattgaatttaggtttgct | 2454 | | |
| ABCG2 | 25 | intron6 + 3287 | gtcaggctgaactagagcaa A/G caatctaaaggcaagaatag | 2455 | | |
| ABCG2 | 26 | intron1 + 179 | ttcatttttgtagcaccagc T/C tgttatttaggtatctttct | 2456 | | |
| ABCG2 | 27 | intron8 + 5677 | gcacttggactttgctttgc T/C acatacttgcattgctctgc | 2457 | | |
| ABCG2 | 28 | intron8 + 5974 | tatactaataaatggtgtgt A/T taagtttttatctctaattg | 2458 | | |
| ABCG2 | 29 | intron10 + 1908 | gacgcttatgtgcagcctat G/T ttgatgtctggaaaggctga | 2459 | | |
| ABCG2 | 30 | intron10 + 2094 | ccctgagggctgaggtatct G/A gattatttccagacttgcta | 2460 | | |
| ABCG2 | 31 | intron11 + 20 | tgtgagtaggtctttgttct A/G ggaacggggctgtccagcag | 2461 | | |
| ABCG2 | 32 | intron11 + 1447 | tgttcttcaaggaaagcccc C/T gtcaaagaaggaaaagaagc | 2462 | | |
| ABCG2 | 33 | intron12 + 49 | atgtctttagtcttgcctat G/T ggtgaagtcagttgcacctt | 2463 | | |
| ABCG2 | 34 | intron12 + 1566 | tatgcagttacatggacaga C/T acaacattggagaccgaggg | 2464 | | |
| ABCG2 | 35 | intron13 + 40 | gctctgataaggaattgttt C/T tttccttcatttcttcctgc | 2465 | | |
| ABCG2 | 36 | intron13 + 1823 | ttactcaagcaggcctgact C/T ttagtatttgctttttgtag | 2466 | | |
| ABCG2 | 37 | intron14 + 497 | ctaatgaaaacaaacaagaa T/C gaaagattgtcactgtaaat | 2467 | | |
| ABCG2 | 38 | intron14 + 815 | taactctttggaaacttctt A/G aaatttaaaactgtttacct | 2468 | | |
| ABCG2 | 39 | intron15 + 110 | ccaggggcactgaatttttc C/T gagcctacgttttctcatcc | 2469 | | |
| ABCG2 | 40 | intron15 + 566 | gccgcatagtcatgtgttgt T/A gtttttaaattaacttggaa | 2470 | | |
| ABCG2 | 41 | intron15 + 639 | aacaagaaacacttgaataa G/A ttgagaaaaaaccccgtttt | 2471 | | |
| ABCG2 | 42 | intron15 + 1197 | tgagtagctgggattacagg C/T gcccaccaccacacctggct | 2472 | | |
| ABCG2 | 43 | intron16 + 520 | catcaattcaggtcaagaaa T/C agaagattgtagcacacaaa | 2473 | | |
| ABCG2 | 44 | 5′flanking − (996-995) | gttgggatggctacactcac TCAC/Δ aaagcctgatggcccgtttc | 2474 | | |
| ABCG2 | 45 | intron13 + 405 | ctgctagtttattttttttt T/Δ aacatttttaatttatgttt | 2475 | | |
| ABCG2 | 46 | intron13 + (692-702) | tcaatatgtttctgcttatc (T)9-11 aatggttacttaatcctaat | 2476 | | |
| ABCG2 | 47 | intron15 + (645-650) | aaacacttgaataagttgag (A)7-8 ccccgttttcacataatgtt | 2477 | | |
| ABCG4 | 1 | intron1 + 84 | ggcctgggtgtcccatgttc G/A gaaagtcctgcaccagtggg | 2478 | | |
| ABCG4 | 2 | intron2 + 77 | gaacacagaaggtattctga A/G agggcattgacccccatcct | 2479 | | |
| ABCG4 | 3 | intron6 + 679 | tggtgtccctcatgaagtcc C/T tggcacaggggggccgtacc | 2480 | | |
| ABCG4 | 4 | intron7 + 95 | ggcctcctaggggtagagat C/T tcaccgtcgcctgccttccc | 2481 | | |
| ABCG4 | 5 | intron8 + 158 | cttgcccttgggaagtgagt G/A tgaatctaaactgagctctc | 2482 | | |
| ABCG4 | 6 | intron9 + 106 | ccccagaggcattgcaacca A/G tgggtgctaggaagaaccta | 2483 | | |
| ABCG4 | 7 | intron9 + 1089 | aggtacacaacttaatggta C/G aagattctctgtagacctgg | 2484 | | |
| ABCG4 | 8 | intron11 + 1113 | acgtgagacgagataagtga T/C ggtcatatggccagggagga | 2485 | | |
| ABCG4 | 9 | intron11 + 1120 | acgagataagtgatggtcat A/G tggccagggaggaaggggac | 2486 | | |
| ABCG4 | 10 | intron11 + 1173 | gggggacagcttgaacaaga A/G tgtggaggcaggatggacac | 2487 | | |
| ABCG4 | 11 | 3′untranslated + 2758 | gagtgacaggcacatacatg A/C gaacaggccatctcagccct | 2488 | | |
| ABCE1 | 1 | 5′flanking − 158 | aactcagattctcggcacct C/T cagcagctggcttcgccaac | 2489 | | |
| ABCE1 | 2 | intron9 + 237 | ctgaaattatatgcaaattc C/T gtagctttataggaagcaga | 2490 | | |
| ABCE1 | 3 | intron9 + 4203 | ttgtgtaggaagctgataca T/G taatttgacatatgagatgt | 2491 | | |
| ABCE1 | 4 | intron10 + 1811 | ccaagaaacttcagctttct C/T ttcacttaaatataggaaac | 2492 | | |
| ABCE1 | 5 | intron17 + 2301 | atatccagaaacagatggta T/C gtgcagaacaggttgtacag | 2493 | | |
| ABCE1 | 6 | 3′untranslated + 2758 | tggatgattagactgactct G/C agaatattgataagccattt | 2494 | | |
| ABCE1 | 7 | intron1 + (5349-5363) | tttgtctgggttggttgggg (T)13-16 gagactgggtctgactctca | 2495 | | |
| ABCE1 | 8 | intron1 + (5845-5845) | tacatttgtcaaaatttata (T)9-10 gcagataatcatttcatctc | 2496 | | |
| ABCE1 | 9 | intron5 + (836-851) | taaattcacatgattctgta (T)14-16 aggatcctcctgactggcag | 2497 | | |
| ABCE1 | 10 | intron8 + (1153-1169) | tctttcaaacttatatttgc (T)13-17 catagtttcatgtttgatga | 2498 | | |
| ABCE1 | 11 | intron9 + (1023-1024) | ttgctctgtttcaaatctct (T) attcatgggccagcagctcg | 2499 | | |
| ABCE1 | 11 | intron9 + (1023-1024) | ttgctctgtttcaaatctct attcatgggccagcagctcg | 2500 | | |
| ABCE1 | 12 | intron9 + (2338-2348) | agtgtagatggacctcgggg (A)6-9 ctagttaaggaaaagtaata | 2501 | | |
| ABCE1 | 13 | intron9 + (3213-3221) | ttccaattttccattgttac (T)8-9 cttgccagattactcctgaa | 2502 | | |
| ABCE1 | 14 | intron10 + (284-299) | tcctctgcattttggcttct GCAGTATTACTGTAGT/Δ atttg | 2503 | | |
| | | | tcattttcaaattaa | | | |
| ABCE1 | 15 | intron10 + (840-853) | ttttttggtttctttctttc (T)13-14 aatcttggaggaatcttttt | 2504 | | |
| ABCE1 | 16 | intron16 + (1163-1172) | gattagaaatccaggttaaa (T)9-10 gttttgcacaaaaatattac | 2505 | | |
| ABCE1 | 17 | intron16 + (1372-1382) | taaaatttaatcaaaattga (T)10-11 ctcttagtcctcaaaccctt | 2506 | | |
| CHST1 | 1 | intron1 + 2475 | taaatggagaaaataacacc G/A acctgatagcattgttgtga | 2507 | | |
| CHST1 | 2 | intron1 + 2612 | aaactccccaagcatgctca C/A ctagatccttaccctaggtc | 2508 | | |
| CHST1 | 3 | intron1 + 3900 | gccctgcccccactcccaga C/G ttgcggccctccagcccctt | 2509 | | |
| CHST1 | 4 | intron1 + 6520 | cctcccccagaggagctggg C/T acactggggccttgtgttgt | 2510 | | |
| CHST1 | 5 | intron1 + 7534 | attgtgtgttggcatactgc T/C cacatggaaggatgctctag | 2511 | | |
| CHST1 | 6 | intron1 + 7911 | ttttccttaggaagaaaaac G/A ccttgctgttttatgcattt | 2512 | | |
| CHST1 | 7 | intron1 + 7963 | aaaacattcatgggggatta G/C tgctggctacgtcagagtca | 2513 | | |
| CHST1 | 8 | intron1 + 8173 | gcgctgccacagatcaggcc G/A aggtgggggacagaaatgcc | 2514 | | |
| CHST1 | 9 | intron1 + 9701 | cccagaattctgaatacagc A/G gcgatgacgggactacgagg | 2515 | | |
| CHST1 | 10 | intron1 + 12132 | aacagatccacaggaccaga C/A agcaaaggggaggaacatgc | 2516 | | |
| CHST1 | 11 | intron1 + 12465 | atgcagggaaggggcttggc G/A caaaactgtcaactgagata | 2517 | | |
| CHST1 | 12 | intron1 + 12561 | atgctccctggtccactttc G/A ctttgagtttcaggtagctg | 2518 | | |
| CHST1 | 13 | intron3 + 529 | ccatggtctgcaggggtcct T/G catgctcaggggattggggt | 2519 | | |
| CHST1 | 14 | intron3 + 617 | agaggacagaggaaagagga C/A cacctggagaactgggcgcc | 2520 | | |
| CHST1 | 15 | intron3 + 796 | aagaggcttccgcagctgtc C/T gcaggttaaatcctggggtg | 2521 | | |
| CHST1 | 16 | intron3 + 818 | caggttaaatcctggggtgc A/G aggaatgtttgttcagctcc | 2522 | | |
| CHST1 | 17 | 3′flanking + 762 | ataactggtacaggtttact G/C gtgtctacactggcagagaa | 2523 | | |
| CHST1 | 18 | intron1 + 7874 | gttttccccttgccttgcct T/Δ cattttcatcacctcatttt | 2524 | | |
| CHST1 | 19 | 3′flanking + (335-349) | cacactgccacacctggcta (T)12-15 ggattttagtagagacgggg | 2525 | | |
| CHST3 | 1 | 5′untranslated − 294 | tccagcgtgccgaccggccc C/G gcagcgcctccatccctccg | 2526 | | |
| CHST3 | 2 | intron1 + 96 | gcgtccaggcgcgcgcgcca G/A actttggagggagaaggggg | 2527 | | |
| CHST3 | 3 | intron1 + 4467 | agagaagaatggggcagagc C/G ggagcagccaggggaggtga | 2528 | | |
| CHST3 | 4 | intron1 + 4853 | ggatgagcactgcccagctg A/G tccctgcccaccttccacag | 2529 | | |
| CHST3 | 5 | intron1 + 4965 | tccactgcagaggggacaca G/C tgaccaggacggaagttggg | 2530 | | |
| CHST3 | 6 | intron1 + 5046 | gggctgtccatctttgtacc C/T ctggttccatcccagtgcct | 2531 | | |
| CHST3 | 7 | intron1 + 5300 | ccttttcttctctaaggcct A/G aagagatgacagaatgctgc | 2532 | | |
| CHST3 | 8 | intron1 + 5354 | agcgcgtggactccacagcg G/A ggtgtggggtggcccctggc | 2533 | | |
| CHST3 | 9 | intron1 + 5428 | gacacgcttcagccctctgt C/G tctattgccccaaatctggc | 2534 | | |
| CHST3 | 10 | intron1 + 5621 | ctgtggcttccctgggccct A/G ggaaatttatcactgaggtt | 2535 | | |
| CHST3 | 11 | intron1 + 6555 | gagtggggcactgctggaag G/C ttctggttcctgctttgttc | 2536 | | |
| CHST3 | 12 | intron1 + 6990 | aaacacactgggccaccccc G/A tccccgcactgtgactacac | 2537 | | |
| CHST3 | 13 | intron1 + 7133 | ctgagggcctgtcctgcagg T/G ttgatgtgtctgaagaggcc | 2538 | | |
| CHST3 | 14 | intron1 + 7161 | gtctgaagaggccccgagaa T/C agaaatctagaacctgccag | 2539 | | |
| CHST3 | 15 | intron1 + 7199 | cagtcacgaagcagtgtcac C/T caccagaggatgaagaactg | 2540 | | |
| CHST3 | 16 | intron1 + 7316 | cttgcatctggtgtaggtgc C/T tgggggtagcgtgcccagga | 2541 | | |
| CHST3 | 17 | intron1 + 7967 | gacaggaaccccaccccgag T/G gatgtctggccctgtgacct | 2542 | | |
| CHST3 | 18 | intron1 + 11412 | gcttgcacttctgattcatt C/T tgcagtcactggctctttgt | 2543 | | |
| CHST3 | 19 | intron1 + 11591 | ccctggaagggcctcactgc G/A gtgactcattacccagcatg | 2544 | | |
| CHST3 | 20 | intron1 + 12541 | acccacacagcatgaatggg G/C ccagccccagcctgcccgct | 2545 | | |
| CHST3 | 21 | intron1 + 12672 | gtagccacagctggggctgt G/C gggtcagggcatggcaaggg | 2546 | | |
| CHST3 | 22 | intron1 + 14809 | ggatgtgtagggtttgggct C/T ggccttaagggatgggtgga | 2547 | | |
| CHST3 | 23 | intron1 + 16161 | gatgctggtcaggcattgtc G/A ttgggatctttaacaccacc | 2548 | | |
| CHST3 | 24 | intron1 + 16385 | tatttagcatgtgggtttca A/C ctttctgttttttcaaaggg | 2549 | | |
| CHST3 | 25 | intron1 + 33638 | gacttgggccacgtccttgg G/C catgaatcttggtctatgtc | 2550 | | |
| CHST3 | 26 | intron1 + 33878 | agcaagaaagtgtgctcccc C/T acagccccactcaggcataa | 2551 | | |
| CHST3 | 27 | intron1 + 34690 | agcacacatggagctttccc G/A cagtgggtttcagcgctccc | 2552 | | |
| CHST3 | 28 | intron1 + 35145 | agggaagccgaagcctcact T/C gctggggcttgcctggcctc | 2553 | | |
| CHST3 | 29 | intron1 + 35340 | tgtgaagttttgcccacagt T/C ggtggccatggttcgcaccg | 2554 | | |
| CHST3 | 30 | intron1 + 35436 | gccactcatgtatggagcaa T/C tgcctttttttcttcctctt | 2555 | | |
| CHST3 | 31 | intron1 + 36150 | ccatagaagaggctgggcct G/T aggaagccagggaagcagga | 2556 | | |
| CHST3 | 32 | intron1 + 36194 | ggtgtggggaggccagcagg G/A gtgtgggcctcagcggggag | 2557 | | |
| CHST3 | 33 | intron1 + 36581 | ctctggtgtttgctgtcaat A/G tgcagagtgctggacaaaac | 2558 | | |
| CHST3 | 34 | intron1 + 37602 | ctggaacagcaacttaaaaa A/T agaaatagtccctggaaggg | 2559 | | |
| CHST3 | 35 | intron1 + 37725 | gggtagccagggcagctccc C/T gacccgcacctgcctttt | 2560 | | |
| CHST3 | 36 | intron1 + 37734 | gcagctccccgacccgca C/G ctgccttttcacccctctcc | 2561 | | |
| CHST3 | 37 | intron1 + 38208 | gccattctagatgcgagtcc C/T gactttggggtgcttgca | 2562 | | |
| CHST3 | 38 | intron1 + 38219 | cgagtcccgactttgggg T/C gcttgcattctgggaaggga | 2563 | | |
| CHST3 | 39 | intron2 + 255 | ctacagctgtgaaaggttag A/G caagatacttaacatttctg | 2564 | | |
| CHST3 | 40 | 3′untranslated + 2202 | acacctcagaggagcctgtg G/A ttaacatttgtaggattatt | 2565 | | |
| CHST3 | 41 | 3′untranslated + 2569 | aggcctcatctggggtaggg C/G caagaggaaagtacagagtg | 2566 | | |
| CHST3 | 42 | 3′untranslated + 2717 | ctggaattcctccttagggc C/T ctgggaagagtattgcttaa | 2567 | | |
| CHST3 | 43 | 3′untranslated + 2753 | cttaacgcaggatgtgctgg G/A tgttttgtttcgggctttta | 2568 | | |
| CHST3 | 44 | 3′untranslated + 2800 | gcttggtgtctttcttgttt C/T atggctgtgtttttgctttt | 2569 | | |
| CHST3 | 45 | 3′untranslated + 3283 | ccgagggctgcccagctctg C/T ttctggtttcctggacaatt | 2570 | | |
| CHST3 | 46 | 3′untranslated + 3327 | ctgtcagatacggcccattg T/C aaacccagagggctgcattt | 2571 | | |
| CHST3 | 47 | 3′untranslated + 3787 | gttccccatgtggaggtcgg A/G ggggctgggactggggaggg | 2572 | | |
| CHST3 | 48 | 3′untranslated + 3860 | ggccctgctaatgtggacag T/C agactttatccctccttctt | 2573 | | |
| CHST3 | 49 | 3′untranslated + 4915 | ccagatgtgcatagaagcca G/A tctctgtcacatacaccgca | 2574 | | |
| CHST3 | 50 | 3′untranslated + 4993 | taaagcaaatttaggctttt G/A tccttctgcaatacatgcac | 2575 | | |
| CHST3 | 51 | 3′untranslated + 5223 | ggaaggagcttcagcaggag G/A tccttcccagaaggttgatt | 2576 | | |
| CHST3 | 52 | 3′untranslated + 5370 | tcatacctgtaatcccagca G/T ttggggaggccaaggtggga | 2577 | | |
| CHST3 | 53 | 3′untranslated + 5545 | ccattcccaaagtcagaaag T/C gaagccagatctcaagggct | 2578 | | |
| CHST3 | 54 | 3′untranslated + 5859 | caaaagcacaaagcagaatt G/C gcaacttcacttgtctca | 2579 | | |
| CHST3 | 55 | 3′untranslated + 5870 | cagaattggcaacttcac T/A tgtctcaagagctccaagat | 2580 | | |
| CHST3 | 56 | 3′untranslated + 5971 | ttccaaggctacagacatgg C/T gccatcctcacaggcctagc | 2581 | | |
| CHST3 | 57 | 3′untranslated + 6208 | atttcatgtctgcatggtac G/A agacaccccttcacggca | 2582 | | |
| CHST3 | 58 | 3′untranslated + 6223 | tacgagacaccccttcac G/A gcatacactgccatggtatg | 2583 | | |
| CHST3 | 59 | 3′flanking + 281 | agacaggagtgttgggccag C/T ggtcagggggcctggggatg | 2584 | | |
| CHST3 | 60 | 3′flanking + 997 | acctcttaaagtatttgagc C/T ggtgcctgtcatcccaacct | 2585 | | |
| CHST3 | 61 | intron1 + 22595 | cgggagcaggaaaaaaaaaa A/Δ gaataagaagaaaagaggct | 2586 | | |
| CHST3 | 62 | intron1 + (35423-35424) | gctcatgctcacagccactc AT/Δ gtatggagcaattgcctttt | 2587 | | |
| NDUFV1 | 1 | intron3 + 670 | ctgggtggagtggggtggca T/C ggagttgaagacccagtcct | 2588 | | |
| NDUFV1 | 2 | intron6 + 160 | tgtgccggccccagccctga C/G catgcatccctttggggacc | 2589 | | |
| NDUFV1 | 3 | intron9 + 27 | accacccttctgcgtagcac G/A gagggtgggtggcatcaagg | 2590 | | |
| NDUFV1 | 4 | 3′flanking + 1111 | tgtaggctgaggtcagcccc A/G atccagtccaaagcccaccc | 2591 | | |
| NDUFV1 | 5 | 3′flanking + 1658 | gaatgcggaagtgctctgtg G/A gcacccaccatgctccgggc | 2592 | | |
| NDUFV1 | 6 | 3′flanking + 1713 | gatctggggcggagggtaca C/T ggggctggcgctgggtgaag | 2593 | | |
| NDUFV1 | 7 | intron4 + 214 | tggtgtaaattttttttttt T/Δ gcttcaaaaatatagtattt | 2594 | | |
| NDUFV1 | 8 | 3′flanking + (772-774) | tgaactcggggttcagggtc TTC/Δ ctgtgaacactggttttgaa | 2595 | | |
| NDUFV2 | 1 | intron1 + 526 | ggaaatgctggctaaataaa C/T ggtatcaaactaactctgaa | 2596 | | |
| NDUFV2 | 2 | intron1 + 6689 | tcgttggatggtagtattgt T/G tgaacaacagaagaaattca | 2597 | | |
| NDUFV2 | 3 | intron1 + 14767 | ccaaatgcatgccagcagag C/T gtggcaggaaggtacacaag | 2598 | | |
| NDUFV2 | 4 | exon2 + 86 | aaggaatttgcataagacag T/C tatgcaaaatggagctggag | 2599 | | |
| NDUFV2 | 5 | intron2 − 29 | cagaagatcttactctctaa T/G gaagctggataacacttttt | 2600 | | |
| NDUFV2 | 6 | intron2 − 168 | tttactttggtaatcatact T/C atcaaatgtgtgtttagaca | 2601 | | |
| NDUFV2 | 7 | intron4 + 677 | aaaccacatactatttgatt C/A tgatgagaatcacataacca | 2602 | | |
| NDUFV2 | 8 | intron4 + 2295 | tatgattcaactttcaaaag A/T gtattgtgatatgaaataga | 2603 | | |
| NDUFV2 | 9 | intron5 + 102 | caacttctgccatcttattg G/A atctgtacttacctagtaat | 2604 | | |
| NDUFV2 | 10 | intron7 + 5466 | tggtaagaggctttaagata A/C caaatgctcagctttcagga | 2605 | | |
| NDUFV2 | 11 | intron1 + (13562-13563) | tactcttaaaattaatcctt (CTT) ttattataagtatacagtct | 2606 | | |
| NDUFV2 | 11 | intron1 + (13562-13563) | tactcttaaaattaatcctt ttattataagtatacagtct | 2607 | | |
| NDUFV3 | 1 | 5′flanking − 606 | aattacgactaacgttgggg A/G cgaactctttgctaaataaa | 2608 | | |
| NDUFV3 | 2 | 5′flanking − 222 | cgccgcgcccccgccacagc G/A cccaggcgcccgcagggcac | 2609 | | |
| NDUFV3 | 3 | 5′flanking − 111 | tggccccaagggaggcactt A/G gccctactggggatgcgcgc | 2610 | | |
| NDUFV3 | 4 | intron1 + 137 | ttgggccgctgaccccgctc C/T gagttacagtatcagcaaaa | 2611 | | |
| NDUFV3 | 5 | intron2 + 152 | tatacaagacacaagatcta T/C aacagattttagaccaaaca | 2612 | | |
| NDUFV3 | 6 | intron2 + 6304 | ttcacagatgaaggggttcc G/A aaatttttgtcaagaaagac | 2613 | | |
| NDUFV3 | 7 | intron2 + 6433 | tcgccttcgtcttcatcctc T/G tccagctcctctgattctga | 2614 | | |
| NDUFV3 | 8 | intron2 + 6563 | cctttgaaaacagagccccc C/T gagttacagtatcagcaaaa | 2615 | | |
| NDUFV3 | 9 | intron2 + 9619 | actatcttctgtgcgcatgc G/A cagagcccaccttgcagagc | 2616 | | |
| NDUFV3 | 10 | intron2 + 9858 | aggatgccagctctttaaat G/A agacatcgtttttgcttaac | 2617 | | |
| NDUFV3 | 11 | intron2 + 11673 | cttggtaggtaagcgcctgt A/G tgtgagccaagtcattcata | 2618 | | |
| NDUFA10 | 1 | 5′flanking − 1734 | tgcaccttgaactgtttact T/C tcctgtaaccatttaccctt | 2619 | | |
| NDUFA10 | 2 | 5′flanking − 1492 | aaaacatccatgcaaacagg T/C tgtgagaagttacgtctgcg | 2620 | | |
| NDUFA10 | 3 | intron3 + 370 | aagactgtgcatgtgccatg G/A agacagagatgtggatgcca | 2621 | | |
| NDUFA10 | 4 | intron3 + 2485 | ttgttattttcttttctctg G/A aatgcagtgatcagttgaca | 2622 | | |
| NDUFA10 | 5 | intron4 + 236 | ctgtgaaagcagattggagc C/T ctggacctcaaacacacgca | 2623 | | |
| NDUFA10 | 6 | intron4 + 1742 | tgtcggcatctgctgagtgt C/T tgctgaagtctgaggactgg | 2624 | | |
| NDUFA10 | 7 | intron4 + 2090 | ggctgggggaaagcagatca T/C gttggctaaaggacaggtgg | 2625 | | |
| NDUFA10 | 8 | intron4 + 3054 | cagctgattatactactgaa A/C cgggataaatgcagcttgat | 2626 | | |
| NDUFA10 | 9 | intron4 + 3066 | ctactgaaacgggataaatg C/T agcttgatgattttcagctg | 2627 | | |
| NDUFA10 | 10 | intron4 + 3377 | gtcacagtttaaatgctgct G/A ttttactctgtgtaagtagc | 2628 | | |
| NDUFA10 | 11 | intron5 + 46 | aagcatctctattttgaatg T/C agatcagcactaaaagccct | 2629 | | |
| NDUFA10 | 12 | intron8 + 1465 | gcaacgcccagttcctggta C/T aggcctcatatccagcgtgc | 2630 | | |
| NDUFA10 | 13 | intron8 + 1809 | cctggaggcacaaggatggc C/A ggggcactcaacttccctct | 2631 | | |
| NDUFA10 | 14 | intron8 + 11226 | gttgtgtgactgtgtggggc A/G tctcacctctcgggctgcag | 2632 | | |
| NDUFA10 | 15 | intron8 + 11319 | atcttgccttccctcctgcc G/A tctgttcaggcttgaatcct | 2633 | | |
| NDUFA10 | 16 | intron8 + 11386 | ccataatcctagcttgaacc C/T tcctttttccctgctgaccc | 2634 | | |
| NDUFA10 | 17 | intron8 + 12301 | acataattattgtaaacatg C/T cgcttaccagtgacattcat | 2635 | | |
| NDUFA10 | 18 | intron8 + 13361 | ccaggccactgattgctttc G/A cattttctagcattttctta | 2636 | | |
| NDUFA10 | 19 | intron9 + 183 | tttctgtgtggaaagctgat G/A aagtcctcagatgacagccc | 2637 | | |
| NDUFA10 | 20 | intron9 + 8669 | aataataatgaccatttctg G/T aaattcatagaattcctttt | 2638 | | |
| NDUFA10 | 21 | intron9 + 8028 | gaggacattccacagaacgt G/A tgactattagagcagaaggt | 2639 | | |
| NDUFA10 | 22 | intron9 + 10742 | ctggaggagaggggtggagc C/G agttcagccagcactggggt | 2640 | | |
| NDUFA10 | 23 | intron9 + 10985 | agaaagggttacacaggagc A/G cacttctcagggagtggtgt | 2641 | | |
| NDUFA10 | 24 | intron9 + 10989 | agggttacacaggagcacac T/C tctcagggagtggtgtgacg | 2642 | | |
| NDUFA10 | 25 | intron9 + 12601 | ctgtgaatcctctcacctgc G/A tgaagggcctggctgcctct | 2643 | | |
| NDUFA10 | 26 | intron9 + 13908 | cacattgttatgtaaccaag C/T ctggaattgcagtgtgaaga | 2644 | | |
| NDUFA10 | 27 | intron9 + 13911 | attgttatgtaaccaagcct G/T gaattgcagtgtgaagaact | 2645 | | |
| NDUFA10 | 28 | intron9 + 14064 | tcttgactattagaaaccct A/G tcagataaattttaaaacag | 2646 | | |
| NDUFA10 | 29 | intron9 + 14184 | tggctttggttgggaacagc G/A agagatacagaaccgacggt | 2647 | | |
| NDUFA10 | 30 | intron9 + 16487 | cttgaagctgatcgttccct C/A cttgaagctgatcgttccct | 2648 | | |
| NDUFA10 | 31 | intron9 + 16779 | gccagacgtgactgctttag G/A ttcctcatgacattcagacc | 2649 | | |
| NDUFA10 | 32 | intron9 + 17663 | ttccaaatcaccccagaact T/G tgcagtattttgaagctcct | 2650 | | |
| NDUFA10 | 33 | 5′flanking − (1668-1659) | gtaaaattgttttaactaga (C)9-11 ttcctaaaccaaggtataaa | 2651 | | |
| NDUFA10 | 34 | 5′flanking − (1355-1334) | ctgtatccattggaaggcac (A)15-21 tgcaaaggaaacaaggcaaa | 2652 | | |
| NDUFA10 | 35 | intron1 + (46 − 61) | tggcggggtggcagggtggc GGGGTGGCGGGGTGGG/Δ gag | 2653 | | |
| | | | AAGCTGATCGT/Δ gtccaagatagttgctagga | | | |
| NDUFA10 | 36 | intron4 + 2486 | ctcactggaacttttttttt T/Δ aatttaatttttaaaatttt | 2654 | | |
| NDUFA10 | 37 | intron7 + (1600-1601) | cacttccattctgactgtta (A) cggtgtgattcttcctgcca | 2655 | | |
| NDUFA10 | 37 | intron7 + (1600-1601) | cacttccattctgactgtta ctgtagacattttaaaccta | 2656 | | |
| NDUFA10 | 38 | intron9 + 1054 | gcgcgtgctgtttctccctt A/Δ tctgtccttgtacacgtgtg | 2657 | | |
| NDUFA10 | 39 | intron9 + (8161-8172) | tttctcgctttctgggagac (T)10-12 aatgttgaaaatatgtgttt | 2658 | | |
| NDUFA10 | 40 | intron9 + (8646-8647) | aattcccccattgcttctct (TT) ctgtagacattttaaaccta | 2659 | | |
| NDUFA10 | 40 | intron9 + (8646-8647) | aattccccattgcttctct ctgtagacattttaaaccta | 2660 | | |
| NDUFA10 | 41 | intron9 + (16503-16523) | ccctccttgaagctgatcgt TCCCTCCTTG | 2661 | | |
| | | | AAGCTGATCGT/Δ gtccaagatagttgctagga | | | |
| NDUFA10 | 42 | intron9 + (17905-17936) | caaatatatgtatacatgta (CA)12-18 tccttcatgaaaactctttc | 2662 | | |
| MGST1 | 37 | 5′flanking − 1376 | ttaataaatgtttattcaat T/G aaaccaactgctaatattct | 2663 | | |
| MGST1 | 38 | intron1A + 147 | cctggagattttaactttct G/A cgaagtttttaaaaaacaact 2664 | | | |
| MGST1 | 39 | intron1B + 36 | ggagaaggggaccgcatgca A/G agggtggcaggcagggaggg | 2665 | | |
| MGST1 | 40 | intron1C + 456 | ccccttgggacggttctcac C/T tgtgccccacttccccagtc | 2666 | | |
| MGST1 | 41 | intron1C + 719 | gcccgcaagcattgctgtat A/G gcacccaggcctccagtgag | 2667 | | |
| MGST1 | 42 | intron1C + 985 | cgagtaaaatttttctaccg C/G tttgttttagagtggtgtct | 2668 | | |
| MGST1 | 43 | intron2 + 3083 | aaaaaatttgtagatatggg T/G actccctatgttgcccaggc | 2669 | | |
| MGST1 | 44 | intron2 + 3106 | tccctatgttgcccaggctg A/G tcttgaattcttgggctcaa | 2670 | | |
| MGST1 | 45 | intron3 + 1703 | ttctcttctaagaagaagtc T/C gtgcagatacttagcacaaa | 2671 | | |
| MGST1 | 46 | intron3 + 2557 | tccagcatcttccctttcca T/C ttttaagttagacttttttt | 2672 | | |
| MGST1 | 47 | intron3 + 3032 | agagacatttagaatatatt C/A cctttaaaggtagagaataa | 2673 | | |
| MGST1 | 48 | intron3 + 3045 | atatattccctttaaaggta G/C agaataacccttcactgaga | 2674 | | |
| MGST1 | 49 | intron3 + 3289 | ggtttatagtgttccccccc T/A ccccgcccccaaaagaccca | 2675 | | |
| MGST1 | 50 | intron3 + 3885 | gaagctgccgctccaggaag G/C agtctgtcgttggagaagag | 2676 | | |
| MGST1 | 51 | intron3 + 3976 | ggaaagctggggaactgttt G/T cctggaacagagtctcaaaa | 2677 | | |
| MGST1 | 52 | intron3 + 4298 | tgtcaactgcgtaacacagg C/T gtagaagtggacattgtttt | 2678 | | |
| MGST1 | 53 | intron3 + 4519 | tttaatagaaaatggtattc C/T tgtcttttctttcccatctc | 2679 | | |
| MGST1 | 54 | 3′untranslated + 603 | gggtaaacccattttgaata T/C tagcattgccaatatcctgt | 2680 | | |
| MGST1 | 55 | 3′flanking + 147 | tatttgctttccttctctct C/T tgttttctttttctctgaaa | 2681 | | |
| MGST1 | 56 | 3′flanking + 237 | cagcacgtttttcctatgaa C/T aagacattctccaaataact | 2682 | | |
| MGST1 | 57 | 3′flanking + 1318 | tggctctgtgtgcatgaaca T/C gcacgcgtgcacgcgcacac | 2683 | | |
| MGST1 | 58 | 3′flanking + 1331 | atgaacatgcacgcgtgcac G/A cgcacacacacacacacaca | 2684 | | |
| MGST1 | 59 | intron1C + (904-923) | tgcgattatctttggtaatt (A)18-20 ggcaaatcagtccaaatttg | 2685 | | |
| MGST1 | 60 | intron1C + (3433-3434) | ccccttcaatactagaacaa (AA) gcagacacattaaatgttac | 2686 | | |
| MGST1 | 61 | intron1C + (3433-3434) | ccccttcaatactagaacaa gcagacacattaaatgttac | 2687 | | |
| MGST1 | 62 | intron1C + 5146 | actatttcaatttttttttt T/Δ ggagggggagacagagtctc | 2688 | | |
| MGST1 | 63 | intron2 + (552-563) | cccagcattataagaatgac (T)9-13 aagtgcagatgtggggaggg | 2689 | | |
| MGST1 | 64 | exon3 + (172-173) | tagcatttggcaaaggagaa AA/Δ tgccaagaagtatcttcgaa | 2690 | | |
| | | | agaaaactggatgtctgaaa TTGACA/GTCCAATAT cactg | | | |
| MGST1 | 65 | intron3 + (152-158) | TGGCTCACAT) agtcatcctctttgtgagac | 2691 | | |
| MGST1 | 66 | intron3 + (2198-2200) | ggattttagattcctcccta CTA/Δ ttctttccgaccttccaccc | 2692 | | |
| MGST1 | 67 | intron3 + (2571-2580) | tttccatttttaagttagac (T)9-10 cacctctctcgttacttcag | 2693 | | |
| | | | tcctcttcatgtctctatgt (GAGATGTTG | | | |
| MGST1 | 68 | intron3 + (4682-4683) | TGGCTCACAT) agtcatcctctttgtgagac | 2694 | | |
| MGST1 | 69 | intron3 + (4682-4683) | tcctcttcatgtctctatgt tgctctggagttgggcaact | 2695 | | |
| MGST1 | 70 | 3′flank+ (1359-1360) | acacacacacacacacacac CC/Δ tgctctggagttgggcaact | 2696 | | |
| MGST1 | 71 | 3′flank+ (1889-1891) | ttagaatagtttctaactat ACT/Δ tttactcccaagagaagctt | 2697 | | |
| HMG17L1 | 1 | 3′untranslated + 864 | ctttctgatttttgatagtc G/C gttgaagaagggagtttgaa | 2698 | | |
| UGT2A1 | 1 | 5′flanking − 1602 | ataacatcttctgcagagaa A/C cttcaatggaaatacactca | 2699 | | |
| UGT2A1 | 2 | 5′flanking − 1480 | tacagattatctttggtgat G/C ggagagcttagaagagacat | 2700 | | |
| UGT2A1 | 3 | 5′flanking − 1406 | atttcagaagatttattaac A/T tgaaaaggatcactctgctt | 2701 | | |
| UGT2A1 | 4 | 5′flanking − 1388 | acatgaaaaggatcactctg C/T ttattcacagacatatgcat | 2702 | | |
| UGT2A1 | 5 | 5′flanking − 935 | aaattattcaatctctttgg G/A cagtggtttctttttctttg | 2703 | | |
| UGT2A1 | 6 | 5′flanking − 287 | cctgaatgtagagttgagat G/A tacagaagctttatccaatt | 2704 | | |
| UGT2A1 | 7 | 5′flanking − 128 | gagaagtaagacacattacc C/T ataaatctgtaaatctccta | 2705 | | |
| UGT2A1 | 8 | intron1 + 535 | cattgatcagggtgatttat C/T catgctaagcttatttaatt | 2706 | | |
| UGT2A1 | 9 | intron1 + 642 | tatattgatcatgttgatac A/C tttatacacatatttgtcta | 2707 | | |
| UGT2A1 | 10 | intron1 + 1221 | ttttaatctaataagcaatt C/G aggaccatctaaagggaaat | 2708 | | |
| UGT2A1 | 11 | intron1 + 1448 | aggtgcttacaggcaacatc C/T acatagcagtctgtggctgg | 2709 | | |
| UGT2A1 | 12 | intron1 + 2000 | gacacattagcttcttttct A/G cagatctctgttctaaaaca | 2710 | | |
| UGT2A1 | 13 | intron1 + 3118 | cttaaaattctttaatgaaa T/G cattgcaacaaatttatatc | 2711 | | |
| UGT2A1 | 14 | intron1 + 3191 | ataaatagaacaactcccta A/T gtttacttctctgcagtgga | 2712 | | |
| UGT2A1 | 15 | intron1 + 3770 | atcaccagataatttactat C/T cattaaggagtaggtcatca | 2713 | | |
| UGT2A1 | 16 | intron1 + 4584 | tgattggttagaatctttga A/C aaatcttctagtatcattcc | 2714 | | |
| UGT2A1 | 17 | intron1 + 4854 | tactctgtgcattgttaata G/A cctatcacttgtggtctgcc | 2715 | | |
| UGT2A1 | 18 | intron1 − 19146 | ctgtttaaattctcattcaa C/T ggccacatggttaaaataaa | 2716 | | |
| UGT2A1 | 19 | intron1 − 18346 | atggcaatatttttagaaat G/A ttaactcccaataatgaata | 2717 | | |
| UGT2A1 | 20 | intron1 − 18218 | tatatcattattttaactta T/G agatagcactagccctaatt | 2718 | | |
| UGT2A1 | 21 | intron1 − 17937 | ctcctaataatttggactca C/T catacttattcagcactatc | 2719 | | |
| UGT2A1 | 22 | intron1 − 12585 | ttccacacagggacaagtca A/G cagaggaaatttttcttgct | 2720 | | |
| UGT2A1 | 23 | intron1 − 11430 | aacaaaggtttattttctta C/G agttctgatggctagacgtc | 2721 | | |
| UGT2A1 | 24 | intron1 − 10761 | tttaaaatatgcatgtattt T/G ccacttttaaaaactatatc | 2722 | | |
| UGT2A1 | 25 | intron1 − 381 | aaatcctccctccttccttc C/T tttccccaggcccactctac | 2723 | | |
| UGT2A1 | 26 | intron1 − 329 | ttccctttctccttttctcc A/G tctctctctcttcctctctc | 2724 | | |
| UGT2A1 | 27 | intron1 + 41 | ttttctcctcagcaaacata T/A aagctaatttcctccatcca | 2725 | | |
| UGT2A1 | 28 | intron2 + 263 | caccttgatactggacttgg T/C gggacagaaaaccagatcat | 2726 | | |
| UGT2A1 | 29 | intron2 + 454 | agaaagcccattgaaataag G/C cagggtttttaggttttaat | 2727 | | |
| UGT2A1 | 30 | intron2 + 554 | aaaaacttttttgagttgac A/T atggtgagtttagtttctga | 2728 | | |
| UGT2A1 | 31 | intron2 + 1113 | ctgcaggcaagctctagtga A/T tgtttattataggaaataat | 2729 | | |
| UGT2A1 | 32 | intron2 + 1304 | gacaaatcagccatgtttta C/T aatagcagacattatgccat | 2730 | | |
| UGT2A1 | 33 | intron2 + 1305 | acaaatcagccatgttttac A/G atagcagacattatgccatt | 2731 | | |
| UGT2A1 | 34 | intron2 + 1367 | atcgatataggctttgggaa A/C tatgaataccaaccatgggt | 2732 | | |
| UGT2A1 | 35 | intron2 + 2074 | aaattttttcttagacctat G/T aatcaaaggaggcatacagt | 2733 | | |
| UGT2A1 | 36 | intron2 + 2164 | attttattagatataactgg A/C atgctaacaattttaaaagc | 2734 | | |
| UGT2A1 | 37 | intron2 + 2298 | taacaatttcagttagcatg A/C gaagagttgtcccttattta | 2735 | | |
| UGT2A1 | 38 | intron2 + 2346 | tttctgtaatggttttgctt T/C catgcttggacttgtaatca | 2736 | | |
| UGT2A1 | 39 | exon3 + 922 | gtgttgtggtgttttctctg G/A gatcaatggtcaaaaacctt | 2737 | | |
| UGT2A1 | 40 | intron3 − 217 | aagcttagaagtgataaata T/C caaaacaataatactatact | 2738 | | |
| UGT2A1 | 41 | exon5 − 194 | aaacaataatactatactgg G/A tagactattagtacaagact | 2739 | | |
| UGT2A1 | 42 | intron5 + 1171 | acggagtccctatggtggga G/A ttcccatgtttgctgatcag | 2740 | | |
| UGT2A1 | 43 | intron5 + 1546 | tttttaaaattcagaaactc A/G gttatggtgtattcttacaa | 2741 | | |
| UGT2A1 | 44 | intron5 + 1547 | ttttaaaattcagaaactca G/A ttatggtgtattcttacaaa | 2742 | | |
| UGT2A1 | 45 | intron5 + 2013 | atcatattcattaccctccc G/T ctattattgtattttgaatc | 2743 | | |
| UGT2A1 | 46 | intron5 + 2318 | aatttagtgctttttcttaa C/T ggaagtaacctgcttaaaaa | 2744 | | |
| UGT2A1 | 47 | intron5 + 2505 | taattgacttttattaatac G/A tacatgttgtataagtcata | 2745 | | |
| UGT2A1 | 48 | intron5 + 2639 | tagactattacaaagttgtt A/G gttgctgacaattttgttca | 2746 | | |
| UGT2A1 | 49 | intron5 + 4009 | gaatccaggctggaactttt C/A ttccagacacaaaccaaaat | 2747 | | |
| UGT2A1 | 50 | intron5 + 4311 | atacagacactgtccttttc G/A tcacaaacatacagatgtgt | 2748 | | |
| UGT2A1 | 51 | intron5 + 4545 | agctcacacagtatcaaaat T/C atttttggaaaaattatgct | 2749 | | |
| UGT2A1 | 52 | intron5 + 4616 | acttttttatgtctacattt G/C atcatactgtgttaagcata | 2750 | | |
| UGT2A1 | 53 | intron5 + 4717 | tgcaagaattatattttctc C/A acgtaactatggccttaaac | 2751 | | |
| UGT2A1 | 54 | exon6 + 1524 | gctatatttttggtcataca A/G tgttgtttgttttcctgtca | 2752 | | |
| UGT2A1 | 55 | 3′untranslated + 1683 | aaggagtttaacaaaaacac G/A tctcccatcctgtttccaaa | 2753 | | |
| UGT2A1 | 56 | 3′flanking + 685 | aatctagaaaataattatca T/C ttttataaaatttttagtca | 2754 | | |
| UGT2A1 | 57 | intron1 − (18967-18965) | ctcccaattagattgattag TAT/Δ gagttcctggggttactggt | 2755 | | |
| UGT2A1 | 58 | intron1 − (18862-18803) | ttgtgtgtatgtgtatgttt (AC)14-17 tattttaatgaattaatatc | 2756 | | |
| UGT2A1 | 59 | intron1 − (17463-17447) | gcttagtatattatatatat (A)16-27 gtaaagaaaatggcagagaa | 2757 | | |
| UGT2A1 | 60 | intron1 − 10860 | attcaatgcaactttttttt T/Δ gtaatggcagaattagaaca | 2758 | | |
| UGT2A1 | 61 | intron2 + (528-538) | ctgttaggaaacaattggtt (A)16-27 gtaaagaaaatggcagagaa | 2759 | | |
| UGT2A1 | 62 | intron2 + (1514-1533) | ttgtgtgtatgtgtatgttt (GT)9-11 tattttaatgaattaatatc | 2760 | | |
| UGT2A1 | 63 | intron5 + (916-917) | gcttagtatattatatatat AA/Δ gtctatatatatagcttagt | 2761 | | |
| UGT2A1 | 64 | intron5 + 1163 | caatatttatgtcatttttt T/Δ ctcacatttactctgtttcc | 2762 | | |
| UGT2A1 | 65 | intron5 + (3819-3838) | agacagacagacacacaaac (AC)8-12 tcaacacatgtaaactactc | 2763 | | |
| UGT2A1 | 66 | intron5 + 4785 | tatcttcaatgaaaataaaa A/Δ caaaaattgtctaatttctg | 2764 | | |
| CATP1 | 1 | 5′flanking − 916 | acagcgtagatgttcaataa G/A tatttgttgtatctgtgaga | 2765 | | |
| CATP1 | 2 | 5′flanking − 843 | tagtgcagcgactatgcctt G/A atgtgtgtgtgtttgggatt | 2766 | | |
| CATP1 | 3 | 5′flanking − 526 | aaatgtgtgcctgtatgtta T/C acatctgtacatatatttcc | 2767 | | |
| CATP1 | 4 | 5′flanking − 172 | acaaacacaactcaaagtat G/A tgtgttattaaaagtagcta | 2768 | | |
| CATP1 | 5 | intron1 + 206 | ttgattcaggcaagttagtc C/G taaatggctttgagagactt | 2769 | | |
| CATP1 | 6 | intron1 + 454 | caacataacaataatttcct G/A taagaaaaatggccattttg | 2770 | | |
| CATP1 | 7 | intron1 + 999 | gtttagcaaggttagatatt A/G atgtggatgttaagacaaaa | 2771 | | |
| CATP1 | 8 | intron1 + 1223 | ttgctagaagctagtaggac C/T agctttataaatacagagat | 2772 | | |
| CATP1 | 9 | intron1 + 1326 | aactagttaggcaacccatg T/C gttttaggggaaaagcaatg | 2773 | | |
| CATP1 | 10 | intron1 + 1336 | gcaacccatgtgttttaggg G/A aaaagcaatgaggtcatgat | 2774 | | |
| CATP1 | 11 | intron1 + 1498 | atagtttgctcttaagaata C/T actctgagaaggtttatagt | 2775 | | |
| CATP1 | 12 | intron1 + 5041 | ttatgctcccgaggagttag C/T tctctaaatgcataaggaga | 2776 | | |
| CATP1 | 13 | intron1 + 9532 | aaaaagttatatagaaatat A/G agtgtcactcctttctagtt | 2777 | | |
| CATP1 | 14 | intron2 + 198 | ttacctcatattaacaccta A/C atattgccacatatcctacc | 2778 | | |
| CATP1 | 15 | intron2 + 961 | aaaaagttatatagaaatat A/G agtgtcactcctttctagtt | 2779 | | |
| CATP1 | 16 | intron2 + 1110 | gtctactagtgttcaactcc T/C ttagatcttagcctgtatca | 2780 | | |
| CATP1 | 17 | intron2 + 1419 | aaagcctaagaaggatgcag T/C gcaatagcctatgtgagaag | 2781 | | |
| CATP1 | 18 | intron2 + 3339 | tatggtttgcaaaaaactta T/C tcgtatatttgtttttttca | 2782 | | |
| CATP1 | 19 | intron3 + 66 | caggaaatgaagttgcactt T/C cctctctaggagcaatgctt | 2783 | | |
| CATP1 | 20 | intron3 + 205 | tcagttttgtcaatttacac A/G atggggatttgggacctttt | 2784 | | |
| CATP1 | 21 | intron3 + 6377 | aatgaatagactttgagtta C/T tggatttttagtggataaat | 2785 | | |
| CATP1 | 22 | intron3 + 7238 | tgaatgtcacattttttaaa G/A tttgtgttccttatctcata | 2786 | | |
| CATP1 | 23 | intron4 + 1016 | ttttattctggattcatgtt T/C gtggaaattgcagtagtcca | 2787 | | |
| CATP1 | 24 | intron5 + 110 | tccacaatgatgagtagagt A/G tcttggcacagttggccttc | 2788 | | |
| CATP1 | 25 | intron6 + 496 | agtgtctgaattataagcca A/G ttttatagttggttgggacc | 2789 | | |
| CATP1 | 26 | intron7 + 1934 | aaagtgaaaggaaattaaaa G/C tgagaacttgagcctgaatg | 2790 | | |
| CATP1 | 27 | intron7 + 2140 | tagaatgtaccaaatgaatc A/G gcatctctgaggatgggacc | 2791 | | |
| CATP1 | 28 | intron7 + 2365 | tgaaatcttctttatcaact C/T gattttcctccagactttac | 2792 | | |
| CATP1 | 29 | intron8 + 88 | gcaaactcctaagttgaagt G/C ttttaggatattttttgact | 2793 | | |
| CATP1 | 30 | intron9 + 534 | tcatattttgtattttaaag G/A ttatctgggttttactgaaa | 2794 | | |
| CATP1 | 31 | intron9 + 1286 | tattcttctgagataaatca T/C tgaaggagtggctatgtggt | 2795 | | |
| CATP1 | 32 | intron11 + 215 | ttcactcctattcctcgcta C/T ttttcttccttatttcttag | 2796 | | |
| CATP1 | 33 | intron11 + 663 | ttcttcttcttttggagctc T/A aaagtagagttcagttaatc | 2797 | | |
| CATP1 | 34 | intron11 + 999 | atcatcactgcatgagagtt A/G gaattatctaactttgtgat | 2798 | | |
| CATP1 | 35 | intron11 + 16727 | tttcttttatttacaaactt A/G tttacttttcaggtgtatga | 2799 | | |
| CATP1 | 36 | intron12 + 48 | ctatcagaacaatattatta T/G tattattttttattacactt | 2800 | | |
| CATP1 | 37 | intron12 + 686 | tatgttttgataaactttgc C/A gtacaaataaagacaattga | 2801 | | |
| CATP1 | 38 | intron12 + 708 | tacaaataaagaaaattgaa A/G tatttccaaataaatcaagt | 2802 | | |
| CATP1 | 39 | intron13 + 418 | tctctggtctccaaaatcat A/G tattttctccctctttacat | 2803 | | |
| CATP1 | 40 | intron13 + 436 | atatattttctccctcttta C/A attttgctgaaacaatcttc | 2804 | | |
| CATP1 | 41 | 3′untranslated + 2130 | gtctttaagaacctaaaaaa C/A ctcttaactcaaaataataa | 2805 | | |
| CATP1 | 42 | 3′flanking + 57 | agtgactaaagtttttctta C/A aaacaagtgtctgaatcaaa | 2806 | | |
| CATP1 | 43 | 3′flanking + 572 | aatacactatggttatttat G/A tgtactataaatggagtgag | 2807 | | |
| CATP1 | 44 | 3′flanking + 788 | atttcctaaatgatcagatg C/T atcatatgaaaaaagaaagc | 2808 | | |
| CATP1 | 45 | 3′flanking + 1356 | aggtgactgacataaatggg G/A gcagaggacataatgaggtt | 2809 | | |
| CATP1 | 46 | 5′untranslated + (189-188) | attttctaatctgtattaaa (A) gcgttccaggtatttttgta | 2810 | | |
| CATP1 | 47 | 5′untranslated + (189-188) | attttctaatctgtattaaa gcgttccaggtatttttgta | 2811 | | |
| CATP1 | 48 | intron4 + (725-726) | tgatctttaatagcggggaa AA/Δ caggcaagtacgctatagtt | 2812 | | |
| CATP1 | 49 | intron4 + (1082-1083) | attgagtcaggaaaccaaaa CA/Δ gtttcaaaaatttgaaaaat | 2813 | | |
| CATP1 | 50 | intron4 + 2301 | aatgtcatgtcttttttttt T/Δ aatgcagagtgtacaaagga | 2814 | | |
| CATP1 | 51 | intron9 + (241-246) | attgtatgtgcatgtgggtg TGTGTG/Δ catgattgtctttgtgatat | 2815 | | |
| CATP2 | 1 | 5′flanking − 2574 | ggataaggcaacccctatgt A/G tcactgctgcaggagaggga | 2816 | | |
| CATP2 | 2 | 5′flanking − 2366 | aacataggaatgtgcagagc C/T ctgtggggattagagaagag | 2817 | | |
| CATP2 | 3 | 5′flanking − 2244 | tgatgatgccagagctttga T/G cattggtgggtatagaaaca | 2818 | | |
| CATP2 | 4 | 5′flanking − 1723 | tctttcagacttcaaaggcc A/G tgatatttcatcagagctgt | 2819 | | |
| CATP2 | 5 | 5′flanking − 1180 | tgcttatttaacaggcataa T/G ctttggtctcctgagccaga | 2820 | | |
| CATP2 | 6 | 5′flanking − 811 | tatgtgcatatgtgtataca G/A gtaaaagtgtgtatatatgt | 2821 | | |
| CATP2 | 7 | intron1 + 7188 | aatcatttgaaatttaagaa A/G aaaatatgttcagagaaaaa | 2822 | | |
| CATP2 | 8 | intron1 + 7331 | gtgaaatgaggaacaaagtg T/C ccacctttttttcctgaata | 2823 | | |
| CATP2 | 9 | intron1 + 7391 | agagagatgtgaaatagtat T/G tttctggggaagtaggggaa | 2824 | | |
| CATP2 | 10 | intron1 + 7886 | ttgttagtagaaagaaaatc G/A aagcctaaaactaaaggaag | 2825 | | |
| CATP2 | 11 | intron1 + 7958 | ttgctattatataatttttt T/A aaaaaaagatttcctaatat | 2826 | | |
| CATP2 | 12 | intron1 + 7959 | tgctattatataattttttt A/T aaaaaagatttcctaatatt | 2827 | | |
| CATP2 | 13 | intron1 + 8036 | ggaaaaaatggggtgaaatt A/T atcaaagggcagcttattaa | 2828 | | |
| CATP2 | 14 | intron1 + 9164 | acattatattctatataaaa G/T agtcagttgaagtaaaaagt | 2829 | | |
| CATP2 | 15 | intron1 + 10123 | tctgtctttcctacttttgt T/G tccagcattgacctagcaga | 2830 | | |
| CATP2 | 16 | intron2 + 193 | tgattaagtatttctttggc G/A aaatttttgatgcttaatag | 2831 | | |
| CATP2 | 17 | intron2 + 1020 | ttgagtaacatttaggccaa G/A tggcagtcataaggaaaaag | 2832 | | |
| CATP2 | 18 | intron2 + 14865 | agaggaattaatcataagag G/A tggcagtcataaggaaaaag | 2833 | | |
| CATP2 | 19 | intron2 + 14931 | gttagttaataacagaaaaa A/T tatcagaaattttaaaaaat | 2834 | | |
| CATP2 | 20 | intron2 + 15417 | ttctaaaataagtaagctaa A/T tattctatattatactacta | 2835 | | |
| CATP2 | 21 | intron2 + 20823 | ttgtataagagatacaaaac A/C aattcctactaggggaaata | 2836 | | |
| CATP2 | 22 | intron2 + 20852 | ctaggggaaataaagcttca G/C taaggaggtggcattaagct | 2837 | | |
| CATP2 | 23 | intron2 + 20930 | atggagagaagcagcagtgt A/G ccacagataaatgaagtgag | 2838 | | |
| CATP2 | 24 | intron2 + 21360 | ttcaaaagctgtatttctca T/C tagtgctttttgtgaataaa | 2839 | | |
| CATP2 | 25 | intron2 + 21467 | tatatacacaatacctgtcc A/G gaagatgtggtataagccaa | 2840 | | |
| CATP2 | 26 | intron2 + 21621 | tatcaatacttatgaagaga A/G ctaactattctaactaggga | 2841 | | |
| CATP2 | 27 | intron2 + 22760 | ttccccacctcctgttggtt C/G tcctcttaaacttctccttg | 2842 | | |
| CATP2 | 28 | intron2 + 23199 | cctatctgcacataacatta C/T aaacttatggcaattataaa | 2843 | | |
| CATP2 | 29 | intron2 + 23218 | acaaacttatggcaattata A/G aactcaatacatattatact | 2844 | | |
| CATP2 | 30 | intron2 + 23330 | gcccttgttcctgttcctct G/A tacctgcctcaactacatag | 2845 | | |
| CATP2 | 31 | intron2 + 23673 | ctggagacggtagctcaaac T/C gaggatgaaaatagacattt | 2846 | | |
| CATP2 | 32 | intron3 + 89 | ggttatcaactggggtaaat T/G tatctctcacaggcaatttg | 2847 | | |
| CATP2 | 33 | intron3 + 224 | tgctaaatattctataatgc A/G caaagaatgatgtaactgaa | 2848 | | |
| CATP2 | 34 | intron4 + 97 | ccctttaaataggcagttac C/A ttttgagaagatacccacta | 2849 | | |
| CATP2 | 35 | intron4 + 568 | ttcatgatccaaattgtggc A/G acgtatttccaggcaacaag | 2850 | | |
| CATP2 | 36 | intron4 + 599 | aggcaacaagatagaagaag A/G aaagaataagaagcaacaaa | 2851 | | |
| CATP2 | 37 | intron4 + 753 | aaaatagacattattccaag T/A taccaagttcccggttaaaa | 2852 | | |
| CATP2 | 38 | intron4 + 781 | ttcccggttaaaaatcccaa G/C tataattactgtggaaggaa | 2853 | | |
| CATP2 | 39 | intron4 + 1196 | aaggaccacaatctagatca G/T cattgctctaaatatgccat | 2854 | | |
| CATP2 | 40 | intron4 + 1229 | tatgccataatatgtgacaa T/C tttgcacctggtatttctac | 2855 | | |
| CATP2 | 41 | intron4 + 1623 | catctagttgaaatggatta G/C attttatttttactacattt | 2856 | | |
| CATP2 | 42 | exon5 + 388 | attctaaagaaactaatatc A/G attcatcagaaaattcaaca | 2857 | | |
| CATP2 | 43 | exon5 + 452 | taatcaaattttatcactca A/G tagagcatcacctgagatag | 2858 | | |
| CATP2 | 44 | intron5 + 165 | ttaatatacacagttcgccc A/T ttaacaacacaggtttaaac | 2859 | | |
| CATP2 | 45 | intron5 + 189 | acaacacaggtttaaactac G/A cgttttcacttctatgcaaa | 2860 | | |
| CATP2 | 46 | intron5 + 191 | aacacaggtttaaactacgc G/A ttttcacttctatgcaaatt | 2861 | | |
| CATP2 | 47 | intron5 + 507 | atataactttgctttcattg C/T aaaaggcaaactgttatatc | 2862 | | |
| CATP2 | 48 | intron5 + 520 | ttcattgcaaaaggcaaact A/G ttatatcatttaaagacttt | 2863 | | |
| CATP2 | 49 | intron5 + 856 | agtcatgataaacctaatag A/G ataaaacaacaaaaaagaaa | 2864 | | |
| CATP2 | 50 | intron5 + 1157 | acagataatttttacttgtt T/C gtgcttttctgtatgatatg | 2865 | | |
| CATP2 | 51 | intron5 + 1226 | ccttgattgtaataatctcc A/C catgccaagagtggggccag | 2866 | | |
| CATP2 | 52 | intron5 + 1228 | ttgattgtaataatctccac A/C tgccaagagtggggccaggt | 2867 | | |
| CATP2 | 53 | intron5 + 1304 | actgttctcgtggtaatgaa G/T aagtctcacaagatctgatg | 2868 | | |
| CATP2 | 54 | intron5 + 1348 | ttataaatgagagttcccct G/A caaaagctctcttgcctgcc | 2869 | | |
| CATP2 | 55 | intron5 + 1407 | ttgctcttccttcatcttcc G/A ccatgattgtgaggcccccc | 2870 | | |
| CATP2 | 56 | exon6 + 521 | gtcatacatgtggatatatg T/C gttcatgggtaatatgcttc | 2871 | | |
| CATP2 | 57 | exon6 + 571 | gggagactcccatagtacca T/C tggggctttcttacattgat | 2872 | | |
| CATP2 | 58 | exon6 + 597 | ctttcttacattgatgattt C/T gctaaagaaggacattcttc | 2873 | | |
| CATP2 | 59 | intron7 + 33 | agaacaaggtaccatgataa C/T gtctttctaagcacacatgc | 2874 | | |
| CATP2 | 60 | intron7 + 267 | caaaataaccaaatgtaaaa T/A gtctccctcccaaactgact | 2875 | | |
| CATP2 | 61 | intron7 + 1260 | gtaatctcacatttctctgc A/G tttacacttggtaaaacttt | 2876 | | |
| CATP2 | 62 | intron7 + 1386 | agtctcaaattaatagccaa G/A agcatgcctttattgtaacc | 2877 | | |
| CATP2 | 63 | intron7 + 1472 | ctttaccacatgacagaatg G/A catgttcttagcaaataata | 2878 | | |
| CATP2 | 64 | intron7 + 1697 | tttacatgttcaattttaga C/A atatgccttagagtagctac | 2879 | | |
| CATP2 | 65 | intron7 + 2273 | ttctcacgtcctatctagcg C/T gattatgacccttagttact | 2880 | | |
| CATP2 | 66 | intron8 + 207 | gtggaagagaattaggtttg T/C actttttagcagggagaaac | 2881 | | |
| CATP2 | 67 | intron8 + 546 | tcgggagaagtttctcccta T/C gtaattagagtaatatttat | 2882 | | |
| CATP2 | 68 | intron8 + 565 | atgtaattagagtaatattt A/C ttttggtaattatctatcta | 2883 | | |
| CATP2 | 69 | intron8 + 668 | taagtaatgtaaattaggat G/T catcagcatttgacagtgcc | 2884 | | |
| CATP2 | 70 | intron8 + 739 | tggagaaccattgagagtca A/G taaacaaagagaatgacttg | 2885 | | |
| CATP2 | 71 | intron8 + 2193 | tgatcacagatccaaatgac A/G taatttctaccatgaacaga | 2886 | | |
| CATP2 | 72 | intron9 + 112 | attttagtaatacaggataa G/C tataattttcttgtattctt | 2887 | | |
| CATP2 | 73 | intron9 + 266 | ttagaggtagtatctgtata A/G ttggatcttataatttagtg | 2888 | | |
| CATP2 | 74 | intron9 + 305 | tgctaagatctgagacaaac C/G cttttgtaattataatcatt | 2889 | | |
| CATP2 | 75 | intron9 + 888 | aggttctgtatgttttttaa T/C aaatgacaaagatatattaa | 2890 | | |
| CATP2 | 76 | intron11 + 10224 | tacacttgttccataaaaaa T/C tcctctatattattcctagt | 2891 | | |
| CATP2 | 77 | intron11 + 10359 | attaatagattcaccgtgag G/C ttcccttaaactttagccta | 2892 | | |
| CATP2 | 78 | intron11 + 10916 | cttatatagaaagaaatcca C/G aaaactattttaccttttat | 2893 | | |
| CATP2 | 79 | intron11 + 10997 | aatatattagtttgaacaag T/C gagacttcactaaatataat | 2894 | | |
| CATP2 | 80 | intron11 + 11018 | gagacttcactaaatataat G/A caatgtatttgcagcactgt | 2895 | | |
| CATP2 | 81 | intron12 + 442 | aacattccaaaacttttaat C/T gactcacagcatgactttta | 2896 | | |
| CATP2 | 82 | intron12 + 445 | attccaaaacttttaatcga C/T tcacagcatgacttttataa | 2897 | | |
| CATP2 | 83 | intron12 + 447 | tccaaaacttttaatcgact G/A acagcatgacttttataata | 2898 | | |
| CATP2 | 84 | intron12 + 907 | aatgaaaagaagctggcaga T/C tgaaacatactgaatgagag | 2899 | | |
| CATP2 | 85 | intron13 + 65 | tatatatatatatatatata C/T acacacacatacatatatta | 2900 | | |
| CATP2 | 86 | intron13 + 870 | aattctgagtatcctatttc G/A atgtatccaatctgtggcac | 2901 | | |
| CATP2 | 87 | intron13 + 1935 | taaaaaaaaaaaaagtctgc T/C tttacagcaattgagccaag | 2902 | | |
| CATP2 | 88 | intron13 + 2261 | aacgaatcctccaaattttt G/C aacttttatttaatcaaaat | 2903 | | |
| CATP2 | 89 | intron14 + 248 | tcaaggataataaccaactt G/A tcaaaaatcagagataatag | 2904 | | |
| CATP2 | 90 | intron14 + 2463 | atttgtttactaatatggaa C/G cttcttcaagacatattttt | 2905 | | |
| CATP2 | 91 | intron14 + 2857 | tcatcatgtatttccaggac A/T cctggcaagatgctcctcag | 2906 | | |
| CATP2 | 92 | intron14 + 11458 | atctccagaggtcctgctgt C/T tcccaaagtcccactgaccc | 2907 | | |
| CATP2 | 93 | 3′untranslated + 2243 | ataataaaacaaactgtagg T/C agaaaaaatgagagtactca | 2908 | | |
| CATP2 | 94 | 3′untranslated + 2404 | tcttaataaaacaaatgagt A/G tcatacaggtagaggttaaa | 2909 | | |
| CATP2 | 95 | 3′untranslated + 2515 | cagagtttgaactataatac T/G aaggcctgaagtctagcttg | 2910 | | |
| CATP2 | 96 | 3′untranslated + 2539 | gcctgaagtctagcttggat A/G tatgctacaataatatctgt | 2911 | | |
| CATP2 | 97 | intron1 + 457 | taattggcaaacataaaaaa (A) caggtgtctcaaagtcacat | 2912 | | |
| CATP2 | 98 | intron1 + 457 | taattggcaaacataaaaaa caggtgtctcaaagtcacat | 2913 | | |
| CATP2 | 99 | intron1 + (7537-7538) | gatcagcattacaaccaaga (G) atggagaatgacattcagga | 2914 | | |
| CATP2 | 100 | intron1 + (7537-7538) | gatcagcattacaaccaaga atggagaatgacattcagga | 2915 | | |
| CATP2 | 101 | intron1 + (10032-10035) | tgtgtgattctatattactt ACTT/Δ gtttcaaatttctctccaca | 2916 | | |
| CATP2 | 102 | intron1 + (10058-10061) | ttcaaatttctctccacaaa TTTA/Δ tttttctattaaattgtaat | 2917 | | |
| CATP2 | 103 | intron2 + (413-423) | acttatttaaaaattctttt (A)11-13 caaaaaacaggattttaaaaa | 2918 | | |
| CATP2 | 104 | intron3 + (1595-1603) | ttgccaagtaattcaagtgc (T)8-10 gtatttaaaacaacttttca | 2919 | | |
| CATP2 | 105 | intron4 + (10-23) | ttcatgggatagtaagtgtt (A)12-14 cctctgtgccactatcagta | 2920 | | |
| CATP2 | 106 | intron5 + (1567-1572) | gtgaatataaattacttgta CTTGTA/Δ aattaaaaaaaaataagtag | 2921 | | |
| CATP2 | 107 | intron5 + (1577-1585) | attacttgtacttgtaaatt (A)9-10 taagtagaataattaagagt | 2922 | | |
| CATP2 | 108 | intron8 + (1939-1941) | ttctctaactccttctactc CTT/Δ atttcaagcagatgcaactg | 2923 | | |
| CATP2 | 109 | intron10 + (3077-3078) | aaattctttatctacttttt (CTT) ttccctctttctctgctttc | 2924 | | |
| CATP2 | 110 | intron10 + (3077-3078) | aaattctttatctacttttt ttccctctttctctgctttc | 2925 | | |
| CATP2 | 111 | intron11 + 11011 | aacaagtgagacttcactaa A/Δ tataatgcaatgtatttgca | 2926 | | |
| CATP2 | 112 | intron12 + (1160-1169) | agcatgacatggtagagatg (A)9-11 gcatttttaacatttgttaa | 2927 | | |
| CATP2 | 113 | intron12 + (1310-1312) | tccatcttaatataaaatgt TGT/Δ ctactcaaaaggagaagtct | 2928 | | |
| CATP2 | 114 | intron13 + (9-34) | tacgagcactaggtatgatg (A)24-27 tatatatatatatatatata | 2929 | | |
| CATP2 | 115 | intron13 + (35-64) | aaaaaaaaaaaaaaaaaaaa (TA)10-21 cacacacacatacatatatt | 2930 | | |
| CATP2 | 116 | intron13 + (1379-1387) | aaaattattcaccacaatac (A)8-10 caaagtaaagttatgaacac | 2931 | | |
| CATP2 | 117 | intron13 + (1916-1928) | aattctcttaaaataatgtt (A)11-13 gtctgcttttacagcaattg | 2932 | | |
| CATP2 | 118 | intron14 + (588-596) | caattatactttacctcttt (A)8-10 ctaatttcaaattcatatat | 2933 | | |
| CATP8 | 1 | 5′flanking − 1413 | aataggggcttaataactct G/C aaacttatgatttctcatat | 2934 | | |
| CATP8 | 2 | 5′flanking − 1345 | gaatttatcctacagatatg A/G ccacacagaaaatgacatat | 2935 | | |
| CATP8 | 3 | intron1 + 38962 | atgaaattagtttaaaaata G/A caaccttaactatactcctc | 2936 | | |
| CATP8 | 4 | intron2 + 253 | acagacttaccaacaaagaa T/G tatccttcccaaaatgtcta | 2937 | | |
| CATP8 | 5 | intron2 + 329 | actcatggtttgcaaattaa C/G tttttaggaaactttatctc | 2938 | | |
| CATP8 | 6 | intron2 + 2568 | ccattctggtgctttctttc G/A tgaaactattttccatcagt | 2939 | | |
| CATP8 | 7 | intron2 + 2679 | ctcttattgctcttcttcca T/G gttttaatctaaataattta | 2940 | | |
| CATP8 | 8 | intron2 + 2753 | caggaaactttcacaaagcc C/A ctaattaatttaagctccct | 2941 | | |
| CATP8 | 9 | intron2 + 3132 | tggtttaatgtaggagagtt T/C gttttaatctaaataattta | 2942 | | |
| CATP8 | 10 | intron2 + 3193 | aatgtcttgggcatatttgc A/G ttcatttggggcattcagtt | 2943 | | |
| CATP8 | 11 | intron2 + 3207 | atttgcattcatttggggca T/C tcagttctactagatacaaa | 2944 | | |
| CATP8 | 12 | exon3 + 334 | gaactggaagtattttgaca T/G ctttaccacatttcttcatg | 2945 | | |
| CATP8 | 13 | intron3 + 76 | agaattttatttttatactt G/A taagtgggcagttacctttt | 2946 | | |
| CATP8 | 14 | intron4 + 2443 | tcaatttcatgttgctctta C/T agttataggtattctaaaga | 2947 | | |
| CATP8 | 15 | intron4 + 67 | taatcacgtctataaagttt C/G tgatattctttaacaaaatt | 2948 | | |
| CATP8 | 16 | intron4 + 91 | tattctttaacaaaattgat T/A attttgtaacgacaaaagaa | 2949 | | |
| CATP8 | 17 | intron4 + 197 | ggtttgaactgcacctgttc G/A cttatatgcagcttttgtcc | 2950 | | |
| CATP8 | 18 | intron4 + 813 | tttaacagaataaaaaaaaa T/A attttgtaacgacaaaagaa | 2951 | | |
| CATP8 | 19 | intron4 + 974 | atatgcaccttaaaaataac C/G tggatttttaaatatgtaat | 2952 | | |
| CATP8 | 20 | intron4 + 1003 | taaatatgtaatgtacataa G/T gaatattatgcatattttgt | 2953 | | |
| CATP8 | 21 | intron6 + 155 | cattaataatcagaataaaa A/G agaaatttagctcctattta | 2954 | | |
| CATP8 | 22 | intron6 + 750 | atccaactggggtttagatt T/G cctctttctgcctctcctcc | 2955 | | |
| CATP8 | 23 | intron6 + 780 | gcctctcctccatctgcacc C/T tctcttttcctcagcaaaca | 2956 | | |
| CATP8 | 24 | intron6 + 1248 | ctatgccctgtaatctcaca C/T ttccctttatttaaaattgg | 2957 | | |
| CATP8 | 25 | intron6 + 1500 | tcgtgtctgtgttagcatat A/G ataactcatcagggtttgtg | 2958 | | |
| CATP8 | 26 | intron6 + 2008 | ataacataaatgagtaaaga A/G tatcaagggcaggaaattag | 2959 | | |
| CATP8 | 27 | intron6 + 2087 | actactctccccatacacac T/C aaaactcatgtgctccccag | 2960 | | |
| CATP8 | 28 | intron6 + 12305 | tcatctatggaggactgcaa T/C cattatcattatttcccaga | 2961 | | |
| CATP8 | 29 | intron7 + 363 | taacaaatgataccagccat C/G atactattctctggtaatag | 2962 | | |
| CATP8 | 30 | intron7 + 411 | cctttattttttgagaacct G/A gtggatgatattaagacgta | 2963 | | |
| CATP8 | 31 | intron7 + 428 | cctggtggatgatattaaga C/A gtatatagatcactgtaata | 2964 | | |
| CATP8 | 32 | intron7 + 634 | aaaattatatatatacatat A/G taatcttacctaagtattca | 2965 | | |
| CATP8 | 33 | intron7 + 1791 | tgtttttttaagggtagtga T/C gtgaatagtaaagcgaattt | 2966 | | |
| CATP8 | 34 | intron7 + 2000 | agttgagcaaattgctctca G/A gtagcataatgtcacttgaa | 2967 | | |
| CATP8 | 35 | intron7 + 2043 | gtttattgatccatttttta A/G tggatcaacattgtagtgag | 2968 | | |
| CATP8 | 36 | intron7 + 2171 | atttattttgagcaaaggtc G/A cgactctcttagaaagcctc | 2969 | | |
| CATP8 | 37 | intron7 + 2173 | ttattttgagcaaaggtcgc G/A actctcttagaaagcctcac | 2970 | | |
| CATP8 | 38 | intron7 + 2179 | tgagcaaaggtcgcgactct C/T ttagaaagcctcacaaatca | 2971 | | |
| CATP8 | 39 | intron7 + 2219 | atttgtaactttaagtctta T/G ataacttatatttacaaaat | 2972 | | |
| CATP8 | 40 | intron7 + 2261 | cagatattaatatatatttt A/T ttattgaaatatgttatttt | 2973 | | |
| CATP8 | 41 | intron8 + 150 | acaaaatttctccatcttgt T/C atatcatcgttgttctgcat | 2974 | | |
| CATP8 | 42 | intron8 + 154 | aatttctccatcttgtaata A/T catcgttgttctgcatttga | 2975 | | |
| CATP8 | 43 | intron8 + 1303 | ttttttttgagatggagtct T/C atatcatcgttgttctgcat | 2976 | | |
| CATP8 | 44 | intron8 + 1372 | aagctccgcctcccaggttc T/G ccacccttctcttaaagaaa | 2977 | | |
| CATP8 | 45 | exon9 + 1272 | tccttcttgtttcaacttct A/G tatttccctctaatctgcga | 2978 | | |
| CATP8 | 46 | intron10 + 63 | tcacagatttgatttaataa A/T tacttatcaaatcttcctat | 2979 | | |
| CATP8 | 47 | intron10 + 911 | cttgcccaatatcctaccaa G/T gtattattaaacggcatgga | 2980 | | |
| CATP8 | 48 | intron10 + 972 | tcctagtttccttgaagata G/A gctacaactttagtaaactt | 2981 | | |
| CATP8 | 49 | intron10 + 1101 | tccctggtcctgtgttgtcc A/T gtagtgaagacctgaaagag | 2982 | | |
| CATP8 | 50 | intron10 + 1103 | cctggtcctgtgttgtccag T/C agtgaagacctgaaagagag | 2983 | | |
| CATP8 | 51 | intron10 + 2027 | cccattttcatgagtggcta A/G gttttgtcccgtttcaaact | 2984 | | |
| CATP8 | 52 | intron10 + 2028 | ccattttcatgagtggctaa G/A ttttgtcccgtttcaaacta | 2985 | | |
| CATP8 | 53 | intron10 + 2148 | gtattttggaaagaaaatgt A/G ggtggaagagaaatatttta | 2986 | | |
| CATP8 | 54 | intron10 + 2214 | atatacagaatttcatacac T/C aatttcttaaattcctaaat | 2987 | | |
| CATP8 | 55 | intron10 + 2316 | taaatattttagtttgagac T/G tctttaaatataatggaatg | 2988 | | |
| CATP8 | 56 | intron10 + 2372 | tgtatttggcaaatgtattt G/T ttaatatttcaaaaactatt | 2989 | | |
| CATP8 | 57 | exon11 + 1557 | cagaacagaaattactcagc A/G cacttgggtgaatgcccaag | 2990 | | |
| CATP8 | 58 | intron11 + 147 | tttcttagaattattttgat A/C tttcaataacatcattaata | 2991 | | |
| CATP8 | 59 | intron11 + 10339 | aaaaaactgcattttagtgg G/C ttagctagaaaagatttgtc | 2992 | | |
| CATP8 | 60 | intron11 + 10358 | ggttagctagaaaagatttg T/G ctcatatacacaataaatta | 2993 | | |
| CATP8 | 61 | intron11 + 10538 | caacagaggatcaatgtaaa T/G gaaatctcttaaattaaaca | 2994 | | |
| CATP8 | 62 | intron12 + 55 | ataaatattaatgttaaata C/T taaagactgaatgcaattaa | 2995 | | |
| CATP8 | 63 | intron12 + 1802 | taaaatgaatcggtaaaaca T/G tcatgtataaatcactgtca | 2996 | | |
| CATP8 | 64 | intron12 + 2612 | ataggcatataatactcttt C/A ttccctctgtatatagggag | 2997 | | |
| CATP8 | 65 | exon13 + 1833 | aacagctgtggagcacaagg G/A gcttgtaggatatataattc | 2998 | | |
| CATP8 | 66 | 5′flanking − (1590-1587) | atatacataacatataccta TATC/Δ tatgttatgtgtctgcttat | 2999 | | |
| CATP8 | 67 | 5′untranslated − (11-28) | agcatcagcaacaattaaaa ATATTCACT | 3000 | | |
| | | | TGGTATCTG/Δ tagtttaataatggaccaac | | | |
| CATP8 | 68 | 5′untranslated − (4-7) | tattcacttggtatctgtag TTTA/Δ ataatggaccaacatcaaca | 3001 | | |
| CATP8 | 69 | intron4 + (213-214) | cctgttcgcttatatgcagc (T) ttttgtccaaccaaacagaa | 3002 | | |
| CATP8 | 70 | intron4 + (213-214) | cctgttcgcttatatgcagc ttttgtccaaccaaacagaa | 3003 | | |
| CATP8 | 71 | intron4 + 505 | tataactttctctttataaa G/Δ atgcaaaatgttatagcatt | 3004 | | |
| CATP8 | 72 | intron4 + 616 | aaaaataaatgaagtggagg A/Δ aaaaaaatgatttcaagttt | 3005 | | |
| CATP8 | 73 | intron4 + (804-812) | acatccatgtttaacagaat (A)9-11 tattttgtaacgacaaaaga | 3006 | | |
| CATP8 | 74 | intron4 + 855 | gagattgtttaaccaaatta G/Δ gaaactattattccaacacac | 3007 | | |
| CATP8 | 75 | intron7 + (619-628) | ttttatatatgaattaaaat (AT)4-5 catatataatcttacctaag | 3008 | | |
| CATP8 | 76 | intron7 + (1773-1779) | attttctatattatgaactg (T)7-8 aagggtagtgatgtgaatag | 3009 | | |
| CATP8 | 78 | intron8 + (1270-1290) | tagtgtgccacccttctctc (T)19-23 gagatggagtctcgctctgt | 3010 | | |
| CATP8 | 79 | intron10 + 665 | ttctttcttaactcaaaggc T/Δ tttttttttccatgtgacac | 3011 | | |
| CATP8 | 80 | intron11 + (247-250) | aaaaatcttaaggcacacac TGAT/Δ tgacagttgccttgattgta | 3012 | | |
| CATP8 | 81 | intron12 + (1622-1630) | aaataaattgttggcatcta (T)8-10 atttttctaagggtcgctgt | 3013 | | |
| CATP8 | 82 | 3′untranslated + (2464-2465) | gagaaaagcctgatgccttt A/Δ aaaaaaaatgaaacactttg | 3014 | | |
| CAT1 | 1 | 5′untranslated − 127 | gcagctcggactcagctccc G/A gagcaacccagctgcggagg | 3015 | | |
| CAT1 | 2 | 5′untranslated − 20 | gaaggcctcagcccccagcc A/G ctgggctgggcctggcccaa | 3016 | | |
| CAT1 | 3 | intron3 + 150 | caatagaacaaccttttctc G/A ggctcatgccgccctgaccc | 3017 | | |
| CAT1 | 4 | intron4 + 211 | ttctctggcttcccccactc A/C gttctccagcctgcctgctc | 3018 | | |
| CAT1 | 5 | intron5 + 33 | gagacttcccatgataacct C/T ccagggcttcacccccaaac | 3019 | | |
| CAT1 | 6 | intron6 + 168 | gaaccagatgcccccagcct C/T gactcagtcccagtctccac | 3020 | | |
| CAT1 | 7 | intron1 + (58-71) | ggaagatgggggcctttgtt (A)13-15 gtacatggagaaattaactg | 3021 | | |
| CAT1 | 8 | intron3 + (1306-1319) | aataggttgaggaggagcag (A)12-15 tcaagagtgtggagggggca | 3022 | | |
| CAT2 | 1 | intron4 + 842 | ttgacctccaaaagtgtttg G/A attacaggcatgggccattg | 3023 | | |
| CAT2 | 2 | intron5 + 33 | gtgtgtgtgagcatgcatat C/A tgtgtgtggtggggagtggg | 3024 | | |
| CAT2 | 3 | intron5 + 183 | ccacatccatcattcgagac A/C aactcgtctcagctgccatg | 3025 | | |
| CAT2 | 4 | intron5 + 184 | cacatccatcattcgagaca A/C actcgtctcagctgccatga | 3026 | | |
| CAT2 | 5 | exon7 + 1269 | actagactgctagtgtcctc C/T ggtgagcccagtcccatagg | 3027 | | |
| CAT2 | 6 | 3′untranslated + 1792 | ataaatgtgtacatgagtgt A/G tgaacacaaatacataaggt | 3028 | | |
| CAT2 | 7 | 3′flanking + 1386 | tgtagcagcccacatcgcca G/A tgttcacacctgagagagag | 3029 | | |
| CAT3 | 1 | 5′flanking − 580 | ctgtgtcagagacacagaca C/G ggaggtcctggctgccccag | 3030 | | |
| CAT3 | 2 | 5′flanking − 463 | ttcctgagaggcaaatcccc T/C tcccctactcgggaggtgcc | 3031 | | |
| CAT3 | 3 | 5′untranslated − 16 | cctgcccacagctctggctc G/A tcttgccccagtgccatgac | 3032 | | |
| CAT3 | 4 | exon2 + 153 | cctgcccacagctctggctc G/A tcttgccccagtgccatgac | 3033 | | |
| CAT3 | 5 | intron2 + 177 | gcaccaagacccttggcttc T/C tcccactcagagtccaagca | 3034 | | |
| CAT3 | 6 | intron2 + 6201 | gctcatcctctctggtcctt T/G tgccccagcacaggttcctc | 3035 | | |
| CAT3 | 7 | intron3 + 79 | tctgctccacccgtgcaccc G/C caaagaggcaaagagctggg | 3036 | | |
| CAT3 | 8 | exon5 + 723 | tggcgttggctgcagttaac T/A gtgtccattcccttcttcgt | 3037 | | |
| CAT3 | 9 | intron5 + 524 | tcgaagtacaaaggaaagtt T/C aaagagaagcctgagcctgg | 3038 | | |
| CAT3 | 10 | intron7 + 386 | gaccaatgggtttcagactc G/A aagacaaaaattatgtttat | 3039 | | |
| CAT3 | 11 | intron7 + 754 | gcccacgtcagacatgacca G/A aagacaaaaattatgtttat | 3040 | | |
| CAT3 | 12 | intron9 + 81 | attgtcctgtcctctaccca G/A tcaatcacagcactttctcc | 3041 | | |
| CAT3 | 13 | 5′flanking − (661-680) | tacatttggtccccaggggg (G) agcggctgatcaggagagaa | 3042 | | |
| CAT3 | 14 | 5′flanking − (661-680) | tacatttggtccccaggggg agcggctgatcaggagagaa | 3043 | | |
| CAT3 | 15 | intron11 + (211-212) | tctgacttggactgggcaaa AA/Δ gtatggtggtatctggacag | 3044 | | |
| ALDH1A2 | 1 | 5′flanking − 714 | aagggatcatcattctgaga C/G cgaggagagggggactcgaa | 3045 | | |
| ALDH1A2 | 2 | intron1 + 314 | cagggatcctcattctgaga G/Δ aaggcgtcgaacccgcttag | 3046 | | |
| ALDH1A2 | 3 | intron1 + (864-875) | ttttgaactgaagaacttac (T)11-12 ataacgaacgtgacgtctt | 3047 | | |
| ALDH1A2 | 4 | intron1 + 1370 | gcatgagacagaagtttttt A/G ttttatgagggtctctaacc | 3048 | | |
| ALDH1A2 | 5 | intron1 + 1667 | aatacgtgtttcagaattta A/Δ tttggtaagctcttccagttc | 3049 | | |
| ALDH1A2 | 6 | intron1 + 1934 | tcagctctttagtgagactt C/G tgaattttctaagacagcca | 3050 | | |
| ALDH1A2 | 7 | intron1 + (1971-1980) | agcacagtgcacaagcagtc (T)6-11 aaaacgtgaagagcagaagct | 3051 | | |
| ALDH1A2 | 8 | intron1 + 2285 | tactgcaacgcagggggtta T/C tgttttttgtcttgctaaac | 3052 | | |
| ALDH1A2 | 9 | intron1 + 2287 | agcacagtgcacaagcagtc C/T tacttaaaataaatgaccac | 3053 | | |
| ALDH1A2 | 10 | intron1 + 2641 | aggactgtgctttttcaaac T/Δ agatggtgctagtcaaaggag | 3054 | | |
| ALDH1A2 | 11 | intron1 + 3035 | gacttttataattttgcata A/G ctgatattataggaatacac | 3055 | | |
| ALDH1A2 | 12 | intron1 + 3319 | aaagagttatgttttttttt T/Δ atgcatctgatatttatatgg | 3056 | | |
| ALDH1A2 | 13 | intron1 + 3474 | tgtctttttattttattcat T/C taaacttctgttttataacg | 3057 | | |
| ALDH1A2 | 14 | intron1 + 4126 | ccttccaacccatttactta G/C attgtcgttttggtcactaa | 3058 | | |
| ALDH1A2 | 15 | intron1 + 4227 | cataaattgtcagtcaaact A/G catgttaatagaccacttca | 3059 | | |
| ALDH1A2 | 16 | intron1 + 4254 | cggacttcaggttttttttt T/Δ aaatacttttttcataactat | 3060 | | |
| ALDH1A2 | 17 | intron1 + 4357 | tccttccactagatgggcct A/G tggtaccatgtggaattata | 3061 | | |
| ALDH1A2 | 18 | intron1 + 5323 | taatccaggttgcaaataga T/C gtatctggtattttaagtag | 3062 | | |
| ALDH1A2 | 19 | intron1 + 8206 | ttttgacaaacctcctagca T/G ttctttaatttctctgttga | 3063 | | |
| ALDH1A2 | 20 | intron1 + 9565 | agataaactgatgaataatt C/T actctgtgctgctgatagct | 3064 | | |
| ALDH1A2 | 21 | intron1 + (6831-9639) | taaaaacggaatgctgaaaga (AAGA) ccttttttgaattaactact | 3065 | | |
| ALDH1A2 | 21 | intron1 + (6831-9639) | taaaaacggaatgctgaaaga ccttttttgaattaactact | 3066 | | |
| ALDH1A2 | 22 | intron1 + 12731 | atgaaatagaaagggtgagg T/A gtaccttggagagaagtgat | 3067 | | |
| ALDH1A2 | 23 | intron1 + 13642 | cagtgtcataaagatacacc G/A gagatcaaaatgtttcatat | 3068 | | |
| ALDH1A2 | 24 | intron1 + (14173-14176) | tctaaaaaataaataaata AAAA/Δ gagaaagttaagtttaagat | 3069 | | |
| ALDH1A2 | 25 | intron1 + 14538 | actcatttattggttcaaag gcgaaagttaagtttaagat | 3070 | | |
| ALDH1A2 | 26 | intron1 + 14685 | ttggatcaaagccttcttaa A/G ccttaggatatgtattgagg | 3071 | | |
| ALDH1A2 | 27 | intron1 + 14711 | gttgagacattaacttacta A/G tttaaatgaagatgctagtt | 3072 | | |
| ALDH1A2 | 28 | intron1 + (15227-15337) | gaagagcacagtagaaagcc (T)6-11 aaggctagcactactattga | 3073 | | |
| ALDH1A2 | 29 | intron1 + 17236 | atcagtaccatgtgttggga A/G tacaacaattaacttaaagt | 3074 | | |
| ALDH1A2 | 30 | intron1 + 18277 | taatacaactcatttgaagg A/G tttagtcttaaaaaaacgaa | 3075 | | |
| ALDH1A2 | 31 | intron1 + 18734 | ctttgagaccctactggatc T/A taagtgatgtgagatgtggg | 3076 | | |
| ALDH1A2 | 32 | intron1 + 19021 | ttaatcacctaaatgtttaa C/T gaataagttgattttttcta | 3077 | | |
| ALDH1A2 | 33 | intron1 + 21634 | aatcaggatatgggggtttc G/A ttctttattctgccaaaaat | 3078 | | |
| ALDH1A2 | 34 | intron1 + 21730 | catttaaagtagtgatttta A/G taggagttggctgttacagt | 3079 | | |
| ALDH1A2 | 35 | intron1 + 21566 | taggataggtttcaaatgtg C/T tgttgtaggactcttttcca | 3080 | | |
| ALDH1A2 | 36 | intron1 + 26782 | aaaagaagaagaaaaaaaaa A/Δ atggtatgagacttggtagg | 3081 | | |
| ALDH1A2 | 37 | intron1 + 27885 | ggatgatcctacccaaggga T/C tggcactttccagacagtat | 3082 | | |
| ALDH1A2 | 38 | intron1 + 28204 | gcactcaatgttttaactgt C/G cttctaattgtgtggttaaa | 3083 | | |
| ALDH1A2 | 39 | intron1 + 28521 | tctttgttacaattcctaag T/C aggggtatcagataatcctc | 3084 | | |
| ALDH1A2 | 40 | intron1 + 43476 | gaataggaggatacggtcaa G/T taacagagataaaaacctgc | 3085 | | |
| ALDH1A2 | 41 | intron1 + 49334 | gaatctggattctctcatgt G/T agaaatttcactatactgct | 3086 | | |
| ALDH1A2 | 42 | intron1 + 50361 | gagtgactggttcataagtc C/T gtaacaattggtagttaact | 3087 | | |
| ALDH1A2 | 43 | intron1 + 51181 | gtaaaaggaatccttaagaa C/T aactgtcaatctgtgagcca | 3088 | | |
| ALDH1A2 | 44 | intron3 + 654 | ttaacctctcttgagtaaaa G/A gaatacttgagaactcactg | 3089 | | |
| ALDH1A2 | 45 | intron3 + 664 | gtaaaaggaatcattaggaa C/T cagaggcgatggtaaggaat | 3090 | | |
| ALDH1A2 | 46 | intron3 + 712 | aatagactactgctccgttt G/T aaatgtcattctgtgagcca | 3091 | | |
| ALDH1A2 | 47 | intron3 + 1273 | tattcatactatgaaaaagg T/A gtttcatggggaagaaattc | 3092 | | |
| ALDH1A2 | 48 | intron3 + 1743 | atacacctaactgagattcc C/T gttttaaacaatctcaagct | 3093 | | |
| ALDH1A2 | 49 | intron3 + 2591 | tgcagatatataatcattgt A/G gttttactaggaactaggac | 3094 | | |
| ALDH1A2 | 50 | intron3 + 2819 | ctaggaaatagacaaaaatg G/A cagtactagaaatcttttta | 3095 | | |
| ALDH1A2 | 51 | intron3 + 220 | aattgtgctagattaggtgc T/C ggggtacgtatgaagggaca | 3096 | | |
| ALDH1A2 | 52 | intron3 + 280 | cggcttactcctcctgaaca T/C ataagatatactgtttggaa | 3097 | | |
| ALDH1A2 | 53 | intron3 + 461 | cataatggctggttttcagt G/T tctttttatatttcctatgg | 3098 | | |
| ALDH1A2 | 54 | intron4 + 506 | tctacatttctcgaacggcc G/A tgaattacttactaatctgg | 3099 | | |
| ALDH1A2 | 55 | intron4 + 1952 | ttggtccctactccacctgt C/G atttcattattaaaacaaca | 3100 | | |
| ALDH1A2 | 56 | intron4 + 2078 | ctgtgtggcgtggcggtcgg C/T ctcagttttcttttacttcc | 3101 | | |
| ALDH1A2 | 57 | intron4 + 2589 | ttgcgtcagaaacctctctc C/G ggtggtgtctcaaacagatg | 3102 | | |
| ALDH1A2 | 58 | intron4 + (2840-2851) | tttgtatgtgcatagttggc (T)11-13 cacagtgaagtctggagtat | 3103 | | |
| ALDH1A2 | 59 | intron4 + 7231 | aataggatacaaatacacaa A/T gatagtgattcagatcctaa | 3104 | | |
| ALDH1A2 | 60 | intron4 + 7958 | taaaatcgtttttattgtta C/T taggtatataaaatttgcta | 3105 | | |
| ALDH1A2 | 61 | intron4 + 8090 | tctgattttatcactgttta C/T agattgcttagtcatactca | 3106 | | |
| ALDH1A2 | 62 | intron4 + 12823 | tgttagcctgtagctaaatg C/T ttttcaaatatgtgaacggt | 3107 | | |
| ALDH1A2 | 63 | intron4 + 12939 | atgaggtccgacttttaaga T/C ttttgtctacattttcttcc | 3108 | | |
| ALDH1A2 | 64 | intron4 + 14935 | tattgatggagttcttttta T/G aaatggacttttaccttctt | 3109 | | |
| ALDH1A2 | 65 | intron4 + 15321 | gcatttgggtgtctgagaga C/T atatccagaaatatgctatg | 3110 | | |
| ALDH1A2 | 66 | intron4 + 15412 | tttcaagtttatttctgttt T/G aaatggacttttaccttctt | 3111 | | |
| ALDH1A2 | 67 | intron5 + 1888 | aatccaaacatctgtacttt G/T tagtggacaagatttatgtc | 3112 | | |
| ALDH1A2 | 68 | intron7 + 9166 | gaaaagctactttattcaaa G/A ataaaagtattttaagaaaa | 3113 | | |
| ALDH1A2 | 69 | intron7 + 9914 | aagctggagaaaatactagg C/T tttcctcaacagtgatttcc | 3114 | | |
| ALDH1A2 | 70 | intron7 + 18942 | tttggaggggaactaatccc G/A tgacttctaggttatctctt | 3115 | | |
| ALDH1A2 | 71 | intron7 + 19820 | ttcacccctcattttaggtt A/G ggggaggtggcttgctacag | 3116 | | |
| ALDH1A2 | 72 | intron7 + 19826 | cctcattttaggttagggga G/A gtggcttgctacagttttag | 3117 | | |
| ALDH1A2 | 73 | intron7 + 19913 | cgtgaatcattcagtatttt A/G tttaaaaataccagtttgaa | 3118 | | |
| ALDH1A2 | 74 | intron7 + (20110-20111) | catgatttattctctaacta (ACTA) tgctaagtcaaagattctgc | 3119 | | |
| ALDH1A2 | 74 | intron7 + (20110-20111) | catgatttattctctaacta tgctaagtcaaagattctgc | 3120 | | |
| ALDH1A2 | 75 | intron7 + 21857 | acaatgaaaattaagaaagg A/T gaagagggaagaagcagaga | 3121 | | |
| ALDH1A2 | 76 | intron7 + 21929 | tacaagacacaggcatcttt A/G actagtttactgggatctct | 3122 | | |
| ALDH1A2 | 77 | intron7 + 23308 | ggctttgacttcggaaacct G/T tgggttataacaaagtactg | 3123 | | |
| ALDH1A2 | 78 | intron7 + 23554 | gacattggtgaaaaccaggg C/T tgtttaggagtgtcctgtcc | 3124 | | |
| ALDH1A2 | 79 | intron7 + (23701-23703) | catctgagatttgccttgtg GTG/Δ tttaccgagttagtgggtgc | 3125 | | |
| ALDH1A2 | 80 | intron7 + 26479 | gatacatgaacaatttgttt T/C atcctcatgatatctttcaa | 3126 | | |
| ALDH1A2 | 81 | intron7 + 26561 | taaaggccacaatgcagtga T/C tgaaatctccagttacattt | 3127 | | |
| ALDH1A2 | 82 | intron7 + 26662 | tttccttagtccttccatca C/T gaaactaaagctgtcttcca | 3128 | | |
| ALDH1A2 | 83 | intron8 + 76 | tttatatctccacttttgat G/A ggacactagcaaaagatatt | 3129 | | |
| ALDH1A2 | 84 | intron8 + (700-711) | accatctcattcagtgattc (T)11-12 ccctccacttgttgccaggc | 3130 | | |
| ALDH1A2 | 85 | intron8 + 724 | ttttttttccctccacttgt T/C gccaggcagagctgctttcc | 3131 | | |
| ALDH1A2 | 86 | intron8 + 800 | cagattgcttgaatttcagc C/A ccagcttggaatttgcagag | 3132 | | |
| ALDH1A2 | 87 | intron8 + 1251 | gatttctgtgaaaattgaga G/A gatctggcaacctggggctc | 3133 | | |
| ALDH1A2 | 88 | intron8 + 1627 | ggcccctccccaggcaaagc G/A gtgagaacatggctgtttcc | 3134 | | |
| ALDH1A2 | 89 | intron9 + 141 | tggagcgggccaagaggcgc G/A tagtggggagtccctttgac | 3135 | | |
| ALDH1A2 | 90 | intron9 + 778 | aaccagtctggacagatccc T/C tgtagcttgtgaaagtgtag | 3136 | | |
| ALDH1A2 | 91 | intron9 + 801 | tagcttgtgaaagtgtagga A/G gtgaagggctggctcacttc | 3137 | | |
| ALDH1A2 | 92 | intron9 + 868 | tctgaaggcctcgtgtactt T/C agtggggtggggagggccac | 3138 | | |
| ALDH1A2 | 93 | intron9 + 1338 | aatttttgcctctttttact A/G tcaatacaacttgctaagtt | 3139 | | |
| ALDH1A2 | 94 | intron10 + (227-229) | ctatgtgcttatgattatta TTA/Δ gccaacagaacaatcagaat | 3140 | | |
| ALDH1A2 | 95 | intron10 + 316 | ctaaatgtgggtcactggga T/C gttaaccaggagagagaatc | 3141 | | |
| ALDH1A2 | 96 | intron10 + 368 | ctttacatctgtgcaagaga G/A ggacaaggagcaaatcagcc | 3142 | | |
| ALDH1A2 | 97 | intron10 + 660 | gtaaacttgcattgaaatgt G/A gaaagcaggtaaaggaatga | 3143 | | |
| ALDH1A2 | 98 | intron11 + 104 | tggggaataccaaaagcaac C/T aaagttcaccagaaaagggg | 3144 | | |
| ALDH1A2 | 99 | intron11 + 229 | aaacttctaaaagaaatacc A/G tgccagtcagattatgtgct | 3145 | | |
| ALDH1A2 | 100 | intron12 + 117 | catacattcaccaaacattt C/T gtggagcacatgctactata | 3146 | | |
| ALDH1A2 | 101 | intron12 + 691 | gatagggaagatcactgtga A/G ctggaaaaatctgggaaacc | 3147 | | |
| ALDH1A2 | 102 | intron12 + 1934 | catcttgtctagattgcatg T/C ttgtttgtttgtttgtctct | 3148 | | |
| ALDH1A2 | 103 | intron12 + 1973 | ctacttacccccaaaacatg T/A tttctctttcttaaatgacc | 3149 | | |
| ALDH1A2 | 104 | intron12 + 2722 | ccagagtgactccagtatac C/A tcactgcccaggacccacag | 3150 | | |
| ALDH1A2 | 105 | intron12 + 3855 | cacttgaaagcaaccataat T/C gtgaggtttctgatgctgta | 3151 | | |
| ALDH1A2 | 106 | intron12 + 4185 | ttgctttaagcgaaatgaac T/C atacggacaggagaacagcc | 3152 | | |
| ALDH1A2 | 107 | intron12 + 4991 | acaggaacacttagacatgc A/G acccactcccaccctccgtc | 3153 | | |
| ALDH1A2 | 108 | intron12 + (5018-5019) | cccaccctccgtcttggggg (G) aggaaagcacactactgtcc | 3154 | | |
| ALDH1A2 | 108 | intron12 + (5018-5019) | cccaccctccgtcttggggg aggaaagcacactactgtcc | 3155 | | |
| ALDH1A2 | 109 | intron12 + (5051-5052) | actgtcccaaagaactaata (A) ctgaaccagtgctgccttgt | 3156 | | |
| ALDH1A2 | 109 | intron12 + (5051-5052) | actgtcccaaagaactaata ctgaaccagtgctgccttgt | 3157 | | |
| ALDH1A2 | 110 | intron12 + (5300-5302) | ttaaagttttaaaaaaactt CCT/Δ taaaaactactcatgagatg | 3158 | | |
| ALDH1A2 | 111 | intron12 + 5405 | catcccaggacttgctgttc G/C caggtgataaactgcacctc | 3159 | | |
| ALDH1A2 | 112 | intron12 + 5435 | aactgcacctccccaggact C/A ccgctgcactcacatgcagc | 3160 | | |
| ALDH1A2 | 113 | 3′flanking + 449 | tttgggccgggaacaatttt T/C caaggttgtaaagccaaatt | 3161 | | |
| ALDH1A2 | 114 | 3′flanking + 597 | acctgggatattcctgaccc A/C atctggttttcttttaccca | 3162 | | |
| ALDH1A2 | 115 | 3′flanking + 669 | atagagactggaagtcatca T/C gtgcagttcaccgcttctga | 3163 | | |
| ALDH1A2 | 116 | 3′flanking + 1122 | cgtgctccactgagctcctc T/G gtcacaccccattcttgccc | 3164 | | |
| ALDH1A2 | 117 | 3′flanking + 2214 | tgcagctgtaaaaagaaatc T/C gtaaatggtgaccgtactac | 3165 | | |
| ALDH1A3 | 1 | 5′flanking − 1425 | cagtgttagccagccgatat C/T ggtcaaggctgccccgctcg | 3166 | | |
| ALDH1A3 | 2 | 5′flanking − 1379 | ccattatcccctttccccgg C/T ctcagctgtgcactccaggc | 3167 | | |
| ALDH1A3 | 3 | 5′flanking − 1270 | aacttacccctctatccagc T/A ctatccagaaggacaccagg | 3168 | | |
| ALDH1A3 | 4 | 5′flanking − (1214-1213) | acggaggcctcaaaacagga (GGA) aaataaggagacccctcccc | 3169 | | |
| ALDH1A3 | 4 | 5′flanking − (1214-1213) | acggaggcctcaaaacagga aaataaggagacccctcccc | 3170 | | |
| ALDH1A3 | 5 | 5′flanking − 1103 | gcacagcttttgtcaggagt C/T cgtgcctccggtctttgttc | 3171 | | |
| ALDH1A3 | 6 | intron1 + 986 | gccttaactttccccacctt T/G ggcttctcttgatttttgct | 3172 | | |
| ALDH1A3 | 7 | intron1 + 1462 | gtacaggatttcaaaatact G/A caacctttgccagttaaagg | 3173 | | |
| ALDH1A3 | 8 | intron1 + 1661 | cctgttgtcttggtgggtgc G/A caacctttgccagttaaagg | 3174 | | |
| ALDH1A3 | 9 | intron1 + 2360 | agaggatagaagtcccttct A/G atttagagggcctctttctt | 3175 | | |
| ALDH1A3 | 10 | intron1 + 2516 | tgaaaacatattctttttga G/A tttagctgagtggcctgttg | 3176 | | |
| ALDH1A3 | 11 | intron1 + 2624 | cctgagacaccttacagctc C/T gtcctgcttccatgtcattc | 3177 | | |
| ALDH1A3 | 12 | intron1 + 3255 | tttcatctttctacaaatgg G/C cccctcttcctggctgcact | 3178 | | |
| ALDH1A3 | 13 | intron1 + (3643-3656) | gcttcagaggtttttgtggg (T)12-14 aacattctatcaacttttaa | 3179 | | |
| ALDH1A3 | 14 | intron1 + 4265 | ccaaaagccctctcttttaa T/C atgacattaataagacaatt | 3180 | | |
| ALDH1A3 | 15 | intron1 + 5187 | caagatggataagacgtcac C/T taaggtccttagcatgttga | 3181 | | |
| ALDH1A3 | 16 | intron2 + 43 | ctctaagtaattcaattatg G/T atgaccaaaggataaggaaa | 3182 | | |
| ALDH1A3 | 17 | intron2 + 127 | cagggcctgggctagctgcg T/C gaattggcatgtggttctca | 3183 | | |
| ALDH1A3 | 18 | intron2 + (285-300) | aggaaaggttttcttttttc (T)16-17 atcaattatttggacctgga | 3184 | | |
| ALDH1A3 | 19 | intron2 + 778 | cgtgtgcagagtaggcttgg A/G ttttatcttgcccatgagtt | 3185 | | |
| ALDH1A3 | 20 | intron2 + 1216 | actcggtagagtcactcctg A/C ctggtgtcccacatccactc | 3186 | | |
| ALDH1A3 | 21 | intron3 + 81 | accatggggtatgggaaaaa A/C gatcacggtcctggttttgt | 3187 | | |
| ALDH1A3 | 22 | intron3 + 236 | gctcagcttcttgaccaagt T/G gttgtctataggcagttgag | 3188 | | |
| ALDH1A3 | 23 | intron3 + 1467 | ggcccggttgtaggggagga G/T atctcctttctggcctttga | 3189 | | |
| ALDH1A3 | 24 | intron3 + 1725 | ccacatgttccccgggtgag A/G gtagctccctcccagggtaa | 3190 | | |
| ALDH1A3 | 25 | intron3 + 3777 | gccagaagtagatgccccca A/G ttcagctgctgcattactgg | 3191 | | |
| ALDH1A3 | 26 | intron3 + 3829 | caagtcactgggccgttagc G/C tccgtgcctgcaccttgaag | 3192 | | |
| ALDH1A3 | 27 | intron3 + 4299 | tcactttccacagccacact G/A gccagcctggccgagaagga | 3193 | | |
| ALDH1A3 | 28 | intron4 + 84 | agagccccccctgactgttt C/G cctaaggcaccattcccaac | 3194 | | |
| ALDH1A3 | 29 | intron4 + 126 | ccactccctctccaaatggt A/G ctgccaattcttcttctaag | 3195 | | |
| ALDH1A3 | 30 | intron6 + (290-291) | tagagaattttcaggggggg (G) tcaaccaagagggagccaaa | 3196 | | |
| ALDH1A3 | 30 | intron6 + (290-291) | tagagaattttcaggggggg tcaaccaagagggagccaaa | 3197 | | |
| ALDH1A3 | 31 | intron6 + 705 | aacagctggtgatgagccaa T/G tttccactttcctttggtga | 3198 | | |
| ALDH1A3 | 32 | intron7 + 56 | ggggcgtgttatttgacacc C/T gtgagcttttcctttgacag | 3199 | | |
| ALDH1A3 | 33 | intron7 + 1107 | gatgctgttactctccttgg A/G gacagacactgccctgtgga | 3200 | | |
| ALDH1A3 | 34 | intron7 + 1610 | aagagccacacagaaccacc C/G ccctactgggctgttggaat | 3201 | | |
| ALDH1A3 | 35 | intron8 + 1820 | cacctgtaagtggagcggct T/C agaccaaggatcccaggatg | 3202 | | |
| ALDH1A3 | 36 | intron8 + 963 | gagaaaggacaggaggagga C/T acaggctctcaggaaggaaa | 3203 | | |
| ALDH1A3 | 37 | intron8 + 1824 | accattcttatccactaagc G/A tgtcccccaagatcttattc | 3204 | | |
| ALDH1A3 | 38 | intron9 + 2384 | cgcctccctcgcccctcccc C/A tccagtggacttggcagtgg | 3205 | | |
| ALDH1A3 | 39 | intron9 + 24 | atccccctggtgtgtgtgaa A/C ccatggtgcttgtctagggg | 3206 | | |
| ALDH1A3 | 40 | intron9 + 91 | gcctacagggtccctctccg T/C gaaaggaatgctgacctgtc | 3207 | | |
| ALDH1A3 | 41 | intron9 + 219 | actgaggcatgggaggaggg C/G gctattcccagggcagaagg | 3208 | | |
| ALDH1A3 | 42 | intron9 + 495 | ccagacggagagagcctggg G/A caggagaatgtatctccagg | 3209 | | |
| ALDH1A3 | 43 | intron9 + 1472 | ttgacttttgaggccagata C/T accgatttcttccaagagaa | 3210 | | |
| ALDH1A3 | 44 | intron9 + 2038 | taaacaatgtgttcctacgg G/A ctctccagggagtgtggagt | 3211 | | |
| ALDH1A3 | 45 | intron9 + 2124 | caaacagggtctgccagatg G/A catatgcccagcagccaggg | 3212 | | |
| ALDH1A3 | 46 | intron9 + 2154 | agcagccagggaggacctgc G/C gttgggcgaagcccctgtgt | 3213 | | |
| ALDH1A3 | 47 | intron9 + 2197 | cttttggcccctcagggagg G/A gaagagcagctcagcagcat | 3214 | | |
| ALDH1A3 | 48 | intron9 + 2466 | ttcttagttcctcatgtttc C/T ctctagaatgttttcgtgtg | 3215 | | |
| ALDH1A3 | 49 | intron9 + 3655 | gattggtcaagtggcatgca C/T ggtttatgccctctctcctg | 3216 | | |
| ALDH1A3 | 50 | intron9 + 3954 | gggtgcgcttttgacaactg C/G tcagtagcgtgttcacaagc | 3217 | | |
| ALDH1A3 | 51 | exon10 + 88 | tggaatgcgggggctcagcc A/G tggaagacaaggggctcttc | 3218 | | |
| ALDH1A3 | 52 | intron10 + 8 | tgccaaagaggaggtacaag G/A gggctgtggcaaggctacga | 3219 | | |
| ALDH1A3 | 53 | intron10 + 307 | ctctctgattttctaacaca A/C ccggtccccgagtcagtcat | 3220 | | |
| ALDH1A3 | 54 | intron10 + 378 | gtgggttttgccaggaatca G/A ttcaagaacctgtggattca | 3221 | | |
| ALDH1A3 | 55 | intron10 + 975 | aatattgtgtcattccttcc C/G ctggtagttattatggaaac | 3222 | | |
| ALDH1A3 | 56 | intron10 + 1088 | cagtgccaggagccaggggg C/T cttctccagatgactctgag | 3223 | | |
| ALDH1A3 | 57 | intron11 + 105 | ttgtttacattgtatattat A/G taccaagccctgtctcagtg | 3224 | | |
| ALDH1A3 | 58 | intron11 + 274 | agggctccagtacctgtgcc T/G gtggcccctgtgctgtactg | 3225 | | |
| ALDH1A3 | 59 | intron11 + 1088 | cagtgccaggagccaggggg T/A cttctccagatgactctgag | 3226 | | |
| ALDH1A3 | 60 | intron12 + 96 | ctccaatctgctgacacccc G/A tccccccccacaccgcgctc | 3227 | | |
| ALDH1A3 | 61 | intron12 + 5642 | tctgtgctaacgtctgcttc T/C ctcatgccccctaggctggc | 3228 | | |
| ALDH1A3 | 62 | exon13 + 104 | gggctccttcctcaaacatc G/C gacggcggaatgtggcagat | 3229 | | |
| ALDH1A3 | 63 | exon13 + 281 | ataggttgtctgtgaaatcg C/T agtcctgcctggggagggag | 3230 | | |
| ALDH1A3 | 64 | 3′flanking + 743 | gtgagcaggaaactgtagga G/A aaggatattttccctcattt | 3231 | | |
| ALDH1A3 | 65 | 3′flanking + 1145 | gcctcccagctaccccaccc A/G cctcaggaggggtcattcca | 3232 | | |
| ALDH1A3 | 66 | 3′flanking + 1185 | aacctagggtgctgagaatc T/C gggtgggattaccagcaaaa | 3233 | | |
| ALDH1A3 | 67 | 3′flanking + 1600 | acaccacgccctgcaaattg T/C tgggaacttgtcggtggcaa | 3234 | | |
| ALDH1A3 | 68 | 3′flanking + 1847 | caggagccctgcggctgccc C/G ggttctgtgaaatggcagtg | 3235 | | |
| ALDH1L1 | 1 | intron1 + 252 | cgcagcgccaggactggccc G/C ccgaggatctggccggccgc | 3236 | | |
| ALDH1L1 | 2 | intron1 + 544 | ctcaggggctgcgctggagt C/T ccagctccagccactgcgct | 3237 | | |
| ALDH1L1 | 3 | intron1 + 6596 | cagatttttcttaaggtgca C/G tagccactgaggatattttt | 3238 | | |
| ALDH1L1 | 4 | intron1 + 6513 | caattatggtttatcttagg G/A acatgtttatagagatagta | 3239 | | |
| ALDH1L1 | 5 | intron1 + 6478 | atagtattcttacttagctt G/A cattctaaattttgttccct | 3240 | | |
| ALDH1L1 | 6 | intron2 + 240 | gtggcattagggtcctggag A/G agggctatagagaagcccag | 3241 | | |
| ALDH1L1 | 7 | intron2 + 1326 | gaggaggagaccggagagga G/C agccagtccagtcagggccc | 3242 | | |
| ALDH1L1 | 8 | intron3 + 386 | gtcctactctaacttccact G/A ccgctgctctgggcagcaca | 3243 | | |
| ALDH1L1 | 9 | intron4 + 271 | gggcccgttcaatagacaag G/C aaggctaaaggcagggactg | 3244 | | |
| ALDH1L1 | 10 | intron4 + 356 | taggattctatttctctctc C/T ttcactcgttgattctcctt | 3245 | | |
| ALDH1L1 | 11 | intron4 + 608 | gtgctctgataggctgtctc A/C gtcacatgcttcctgctggg | 3246 | | |
| ALDH1L1 | 12 | intron4 + 664 | ggtcacatggcctgagcggc A/G gggcggctcagtcacctggg | 3247 | | |
| ALDH1L1 | 13 | intron4 + 785 | gagggctgcttgcccctgcc C/G gaggacaggctggcagggac | 3248 | | |
| ALDH1L1 | 14 | intron4 + 874 | ccctggggagcccttgctgt T/G tgggcgcagcaggaagagca | 3249 | | |
| ALDH1L1 | 15 | intron4 + 1349 | tccctcaggctcttgctcac G/A tgggcccagactccttggct | 3250 | | |
| ALDH1L1 | 16 | intron4 + 1799 | ctggggctgggaaggaggca G/A ggtcctattgctggggatag | 3251 | | |
| ALDH1L1 | 17 | intron4 + 1815 | ggcagggtcctattgctggg G/A atagcaaccaactggatctc | 3252 | | |
| ALDH1L1 | 18 | intron5 + 272 | aaagcccacagggagataag A/G gtgggagttagggggcaaaa | 3253 | | |
| ALDH1L1 | 19 | intron5 + 301 | tagggggcaaaacgtcagcc G/A tagtgcgagcagtcttcaag | 3254 | | |
| ALDH1L1 | 20 | intron5 + 343 | caaggtgtgagggacagtgc G/A ggtctctggagcaatagcca | 3255 | | |
| ALDH1L1 | 21 | intron6 + 926 | cctgcctgggctactggctt C/T gggggcttcttctcacccac | 3256 | | |
| ALDH1L1 | 22 | exon7 + 41 | aacgctgaacacttcaggcc T/C ggtgcccgagggagacgctt | 3257 | | |
| ALDH1L1 | 23 | intron7 + 305 | cctagaatcagagagaagcc C/T tcccagggagcctgggttca | 3258 | | |
| ALDH1L1 | 24 | intron7 + 837 | gtccggacaaaccccatggg C/T gtggtacccccagccgtgtt | 3259 | | |
| ALDH1L1 | 25 | intron7 + 866 | cccagccgtgttgctgtgtc C/T ggcctaccagagtgaggcgt | 3260 | | |
| ALDH1L1 | 26 | intron7 + 884 | tccggcctaccagagtgagg C/T gtggcagtatggggcctggc | 3261 | | |
| ALDH1L1 | 27 | intron7 + 1118 | aatgttccagaaaatcatgc G/C aggcagtaagggcagaggaa | 3262 | | |
| ALDH1L1 | 28 | intron7 + 1168 | aaagtaaaggttcaggagaa G/A tctagcctggggctgctccc | 3263 | | |
| ALDH1L1 | 29 | intron7 + 1451 | cagggcacccacagcatctg T/C taagtgtcttaagaggaagc | 3264 | | |
| ALDH1L1 | 30 | intron7 + 1489 | caggaatgcaaagaaggcaa T/C taagtgtcttaagaggaagc | 3265 | | |
| ALDH1L1 | 31 | intron7 + 1579 | tcagggtgggaggggagtga G/A gagagaccagctgagcacac | 3266 | | |
| ALDH1L1 | 32 | intron7 + 1691 | ctggctgggctttagcttgc A/C gaaagctccagaacatcttt | 3267 | | |
| ALDH1L1 | 33 | intron8 + 2627 | aaagaggagagccgggggtg C/T ttgtgccaggggttggggga | 3268 | | |
| ALDH1L1 | 34 | intron8 + 2646 | gcttgtgccaggggttgggg G/A aactggttctgattgggcct | 3269 | | |
| ALDH1L1 | 35 | intron8 + 2925 | ctgctgccctccataggtcc C/G agactgaatccttcagagga | 3270 | | |
| ALDH1L1 | 36 | exon9 + 4 | caggtcttgctttgcagagt G/T tttggcagcggatcctcccc | 3271 | | |
| ALDH1L1 | 37 | exon10 + 109 | cagctgttagtgaggaagct G/T cgaggggacgatgaggaggg | 3272 | | |
| ALDH1L1 | 38 | intron10 + (671-672) | tggcattttcctctgtctga (AG) gtcctcttagcccaccctaa | 3273 | | |
| ALDH1L1 | 38 | intron10 + (671-672) | tggcattttcctctgtctga gtcctcttagcccaccctaa | 3274 | | |
| ALDH1L1 | 39 | intron11 + 8 | caccgatggaagtgtgagtg C/A aggcccagcaccccttctcc | 3275 | | |
| ALDH1L1 | 40 | intron11 + 447 | atgagccaaagcacgcctat G/A gtagatacacacgtgaacat | 3276 | | |
| ALDH1L1 | 41 | intron11 + 601 | ctcaaagtgagtcatttgag A/G ggagttaatgaaagactcat | 3277 | | |
| ALDH1L1 | 42 | intron11 + 639 | catctgcaaagggagaggga G/A ggggtagggacacagacagg | 3278 | | |
| ALDH1L1 | 43 | intron12 + 684 | tcctgggagaagagagggtg C/T ggccagatgagccgagaaca | 3279 | | |
| ALDH1L1 | 44 | intron12 + 767 | cgtctaggggtgcgaagcca A/G gttatggcgtggtcccaacg | 3280 | | |
| ALDH1L1 | 45 | intron12 + 1014 | tcataggttccagtcccctt C/T gcaagcccctcaattctaga | 3281 | | |
| ALDH1L1 | 46 | intron12 + 1359 | ctggttctgcctcagctcag C/T acagcagaggctgggtctag | 3282 | | |
| ALDH1L1 | 47 | intron12 + 1734 | ggtggtccaggctgctggtg G/T tcagtagggccggccgagcc | 3283 | | |
| ALDH1L1 | 48 | intron12 + 1901 | ttcagcagcctaactgaatt G/A acaatagaatagtcctgcaa | 3284 | | |
| ALDH1L1 | 49 | intron12 − 470 | gggatggggccacctctcca T/C ctctggagatgccaggctca | 3285 | | |
| ALDH1L1 | 50 | intron12 − 334 | aagggcagcctcttgggcca T/C gacccctttgctgtctgcag | 3286 | | |
| ALDH1L1 | 51 | intron12 − 325 | ctcttgggccatgacccctt T/C gctgtctgcagcaagtgggt | 3287 | | |
| ALDH1L1 | 52 | intron12 − 221 | gaaggaagcgagggaagatc G/C aggaaaggagagagggacag | 3288 | | |
| ALDH1L1 | 53 | intron12 − 4 | cccgcttcccctcaccctgg T/C caggttggcagatctcatgg | 3289 | | |
| ALDH1L1 | 54 | intron13 + 34 | tcccacccagtgtgagcaca T/C gcagactggcccagccatat | 3290 | | |
| ALDH1L1 | 55 | intron13 + 58 | gactggcccagccatatagg A/G gaactccaagggcagcacag | 3291 | | |
| ALDH1L1 | 56 | intron13 + 125 | ccacaactggtggcttggaa T/C gacacctgtttattagcttg | 3292 | | |
| ALDH1L1 | 57 | intron13 + 126 | cacaactggtggcttggaat G/A acacctgtttattagcttgt | 3293 | | |
| ALDH1L1 | 58 | intron13 + 281 | acctgcatccagacgagttc T/G ggtgttgacagagttcagtt | 3294 | | |
| ALDH1L1 | 59 | intron13 + 299 | tcgggtgttgacagagttca A/G ttccgtgtggatgcagggct | 3295 | | |
| ALDH1L1 | 60 | intron14 + 121 | catttatcaaacagccatcc A/G tgtgcttcttgagcacctgc | 3296 | | |
| ALDH1L1 | 61 | intron14 + 157 | gccaggcattgttgtaagga C/T ttgaggacaattgtatttaa | 3297 | | |
| ALDH1L1 | 62 | intron14 + 206 | taatctcccagtaacactgg A/C tcagtcaggtccacggtggg | 3298 | | |
| ALDH1L1 | 63 | intron14 + 219 | cactggatcagtcaggtcca C/G ggtgggaaacaagagtaaac | 3299 | | |
| ALDH1L1 | 64 | intron14 + 2275 | tctcatctgtgatgcatccg T/C cagacctctgctcccagcct | 3300 | | |
| ALDH1L1 | 65 | intron14 + 2431 | agaatgactgagtgatcaga C/G ctagagagccccagccccgg | 3301 | | |
| ALDH1L1 | 66 | intron14 + 2660 | agccaagcatttcttgggga C/T accaagaaaccttgcttggt | 3302 | | |
| ALDH1L1 | 67 | intron14 + 2740 | aactccaccctcaccgtcca T/C gcagctccccaggagcgtca | 3303 | | |
| ALDH1L1 | 68 | intron14 + 2756 | tccatgcagctccccaggag T/C gtcagagggcagaggagggg | 3304 | | |
| ALDH1L1 | 69 | intron14 + 2805 | ccgcacagcaggagaatggc T/C ccaagggagggagggacggg | 3305 | | |
| ALDH1L1 | 70 | intron14 + (3636-3637) | tctcctgggtgtgtgtgggg (G) tgtggggcagctcccctatc | 3306 | | |
| ALDH1L1 | 70 | intron14 + (3636-3637) | tctcctgggtgtgtgtgggg tgtggggcagctcccctatc | 3307 | | |
| ALDH1L1 | 71 | intron14 + 4347 | tccaggacagaaacagcagg C/T gtgagctgcctctcagaggg | 3308 | | |
| ALDH1L1 | 72 | intron15 + 380 | atgtcccttatgtggcttcc A/G agaccagaagtcctggagag | 3309 | | |
| ALDH1L1 | 73 | intron15 + (1055-1056) | gccacaatctgcagctactc (C) tcccagcttgctgctgggct | 3310 | | |
| ALDH1L1 | 73 | intron15 + (1055-1056) | gccacaatctgcagctactc tcccagcttgctgctgggct | 3311 | | |
| ALDH1L1 | 74 | intron17 + 15 | gaaaaggtgcgtggctgggg G/C tggagcagaggaggggctgc | 3312 | | |
| ALDH1L1 | 75 | intron17 + 44 | aggaggggctgctgtgagtg C/T gcctgggacatggcagtgct | 3313 | | |
| ALDH1L1 | 76 | intron17 + 51 | gctgctgtgagtgcgcctgg G/A acatggcagtgctgtccaca | 3314 | | |
| ALDH1L1 | 77 | intron17 + (2224-2223) | ctggtgtcatctcccagact CT/Δ gtcactaaaccacaatatga | 3315 | | |
| ALDH1L1 | 78 | intron18 + 140 | agcgtcatcacaagcatagc G/A tggcaggcagcaggcttagg | 3316 | | |
| ALDH1L1 | 79 | intron19 + (51-52) | tggttcactgggacagcagc GC/Δ ctggctggagggggttggag | 3317 | | |
| ALDH1L1 | 80 | intron19 + 399 | tcaggtcagcctgggcctga C/A catggacaggggccctggag | 3318 | | |
| ALDH1L1 | 81 | intron19 + 1794 | gtcctgtctgggggtcttaa G/C ggagtcatgagacttccaca | 3319 | | |
| ALDH1L1 | 82 | intron19 + 1969 | tgatcggggtgcggtttggg G/T cgacaggacaggagcagaga | 3320 | | |
| ALDH1L1 | 83 | intron19 + 1972 | tcggggtgcggtttggggcg A/G caggacaggagcagagaata | 3321 | | |
| ALDH1L1 | 84 | intron19 + 2083 | tgagaagagcagaggggtgt G/T ccgggtgctcgagtcacacc | 3322 | | |
| ALDH1L1 | 85 | intron19 + 2119 | acacctgtgtctgattaggg C/T tgattaggggtgcagagttt | 3323 | | |
| ALDH1L1 | 86 | intron20 + 1388 | ttaccctcttcccactcccg C/T tggactgtgagttccatgag | 3324 | | |
| ALDH1L1 | 87 | intron20 + 1564 | cccaggaaccaggaacagtg G/A ggagccatcaccccgccctg | 3325 | | |
| ALDH1L1 | 88 | intron20 + 1873 | tcagtgttaaaacatcattt G/A tgtatgtatgaaaaatattg | 3326 | | |
| ALDH1L1 | 89 | intron20 + 2427 | actaggattggatggacttg G/C gatcaggtctcagctctgtc | 3327 | | |
| ALDH1L1 | 90 | intron20 + 2458 | cagctctgtcacctgccaac C/T ggcggccccatttccctcaa | 3328 | | |
| ALDH1L1 | 91 | intron20 + 2544 | ccaggtgggagagccatctg C/T agcgtggtgacacccatcac | 3329 | | |
| ALDH1L1 | 92 | intron20 + 2573 | gacacccatcacacgggtgc C/T gtgacccggtgcttatgtcg | 3330 | | |
| ALDH1L1 | 93 | intron20 + 2574 | acacccatcacacgggtgcc G/A tgacccggtgcttatgtcgg | 3331 | | |
| ALDH1L1 | 94 | exon21 + 33 | agccaactgttttcacagac G/A tggaagaccacatgttcata | 3332 | | |
| ALDH1L1 | 95 | exon21 + 87 | ccttcgggcctgtcatgatc A/G tctctcggtttgctgatggg | 3333 | | |
| ALDH1L1 | 96 | intron21 + 323 | ccatgcattaaaccaccccc C/G acactgagtggcttggaata | 3334 | | |
| ALDH1L1 | 97 | intron21 + 361 | ataatcagagatttatttta C/G tcacggtctaggttcaatga | 3335 | | |
| ALDH1L1 | 98 | intron21 + 478 | gtcttgcgggaggcttcctc C/A gcgtggcagcctcggggttg | 3336 | | |
| ALDH1L1 | 99 | intron21 + 1086 | caacccaatcttgcccccgg C/T gctgcagcccggcacatttt | 3337 | | |
| ALDH1L1 | 100 | intron22 + 235 | gggcctggaggagacactcc A/C gccaggaggcactgggggcc | 3338 | | |
| ALDH1L1 | 101 | intron22 + 313 | atagcagggaggagttggcc G/A tgaagacccaggggcccgtg | 3339 | | |
| ALDH1L1 | 102 | intron22 + 1214 | tgggcccacttatgaatcct G/C cccgagttccctcagctccc | 3340 | | |
| ALDH1L1 | 103 | intron22 + 1226 | tgaatcctccccgagttccc T/C cagctccctcctaaccctag | 3341 | | |
| ALDH1L1 | 104 | intron22 + 1623 | ggggcttcccactgtccaga C/G aaggcggtgggagctgggga | 3342 | | |
| ALDH1L1 | 105 | intron22 + 1698 | attctggggagtcctggccc A/G ctatccactgccagggataa | 3343 | | |
| ABCA1 | 1 | (5′flanking region − 99) | acataaacagaggccgggaa G/C ggggcggggaggagggagag | 3361 | | |
| ABCA1 | 2 | (intron 1 159) | gcggtgttaaatggggagac G/T atgtcctagtacgagctctg | 3362 | | |
| ABCA1 | 3 | (intron 1 506) | aattggctatatgctcccc G/C ggactggagcggcacagtcc | 3363 | | |
| ABCA1 | 4 | (intron 1 5897) | gtacaaaaccctttagcttt T/G gcaaacctcctttaagaccc | 3364 | | |
| ABCA1 | 5 | (intron 1 5929) | ttaagacccgatttaaatgc C/T tccctcctcatgaagctctt | 3365 | | |
| ABCA1 | 6 | (intron 1 5962) | aagctcttctggatccactc T/C ttcccatcactaagttgaaa | 3366 | | |
| ABCA1 | 7 | (intron 1 5985) | cccatcactaagttgaaagt A/C agatccccttctctttactt | 3367 | | |
| ABCA1 | 8 | (intron 1 11416) | ttacagtgccctttatagga G/A agaaagaagaaattgtgtct | 3368 | | |
| ABCA1 | 9 | (intron 1 11935) | tctctgtggagcaaatagag G/A gctgtctgacacttggttcc | 3369 | | |
| ABCA1 | 10 | (intron 1 12281) | gaatgtttgatttgtgaaaa T/A cttaataacagtagtttttt | 3370 | | |
| ABCA1 | 11 | (intron 1 12924) | gtgctgacaatcttatactc T/C aggttgaacctccggggaag | 3371 | | |
| ABCA1 | 12 | (intron 1 13002) | gagcctcaatcacagattct C/G tctagctcacatgaagttaa | 3372 | | |
| ABCA1 | 13 | (intron 1 17715) | ggagcatgactttgtggaag C/T ctctcctcttccacccagag | 3373 | | |
| ABCA1 | 14 | (intron 1 17848) | gagggctgactgtcaccctt T/C gataggagcccagcactaaa | 3374 | | |
| ABCA1 | 15 | (intron 1 21384) | gtgggtgggaggaattggag G/C aggaagcttgcctaagtgtg | 3375 | | |
| ABCA1 | 16 | (intron 1 23063) | ggaggcacctgtgacaccca G/A cggagtaggggggcggtgtg | 3376 | | |
| ABCA1 | 17 | (intron 1 23131) | agtgtgcatatgtgctgacc G/A tgggagcttgtttgtcggtt | 3377 | | |
| ABCA1 | 18 | (intron 2 2801) | aagaaaagtgatttatttca A/G gttgctgatgcttagattgt | 3378 | | |
| ABCA1 | 19 | (intron 2 2830) | tgcttagattgttagagttg C/G aaagatctggcttgcatctt | 3379 | | |
| ABCA1 | 20 | (intron 2 2856) | tctggcttgcatcttgtaca A/G ctgacagaactggggctcag | 3380 | | |
| ABCA1 | 21 | (intron 2 3187) | tgatagctgttgcctgcagc A/G tacggacgttcattgcgcag | 3381 | | |
| ABCA1 | 22 | (intron 2 3190) | tagctgttgcctgcagcata C/T ggacgttcattgcgcagttc | 3382 | | |
| ABCA1 | 23 | (intron 2 3194) | tgttgcctgcagcatacgga C/T gttcattgcgcagttcctgt | 3383 | | |
| ABCA1 | 24 | (intron 2 3204) | agcatacggacgttcattgc G/A cagttcctgtctcctgagat | 3384 | | |
| ABCA1 | 25 | (intron 2 3401) | acataaagcctgtgtgctgc T/C gccaggaagactagaaacgc | 3385 | | |
| ABCA1 | 26 | (intron 2 13927) | gtcaccacatacctggcact A/G tgctaaggctgggaatgcag | 3386 | | |
| ABCA1 | 27 | (intron 3 4163) | ccagcccacttcatcttacc G/A tagttacctccttagagtat | 3387 | | |
| ABCA1 | 28 | (intron 3 4262) | tgtcaaagaggaactaagga T/C gccagggactttctgcttag | 3388 | | |
| ABCA1 | 29 | (intron 3 4306) | ccctctcatcacttctccaa C/T gctggtatcatgaaccccat | 3389 | | |
| ABCA1 | 30 | (intron 5 490) | gatgggcatttgaacttgtt G/A tctttaaaaagtgaaatctt | 3390 | | |
| ABCA1 | 31 | (intron 5 583) | tatctggggagtgggcattt T/G ctgactgaggcattggctgc | 3391 | | |
| ABCA1 | 32 | (intron 5 1051) | ggctacaaaactgtgctttc C/T ttgggcagtaaaagaggcaa | 3392 | | |
| ABCA1 | 33 | (intron 5 3051) | tagagaacaagtctaattct G/A ttttccttgaaatagtcgaa | 3393 | | |
| ABCA1 | 34 | (intron 5 3127) | aagtccatgattttttaggc A/G aaatggcctcctttcctctt | 3394 | | |
| ABCA1 | 35 | (intron 5 5924) | ctttctttcacaaaattgcc C/T cccagagctttctggaaggg | 3395 | | |
| ABCA1 | 36 | (intron 5 6831) | ccagtccctcagccttgcca T/C tgcttatgctggtctggaaa | 3396 | | |
| ABCA1 | 37 | (intron 5 12678) | gctcaccgctctgctcaccc G/C accctctggccatctcctct | 3397 | | |
| ABCA1 | 38 | (intron 5 14214) | cagcttggtcccagaggcct G/A gacctgggtcccagaggtcc | 3398 | | |
| ABCA1 | 39 | (intron 5 14257) | gctggttccccggcttggtc C/T cagaggcctggatgtgtggc | 3399 | | |
| ABCA1 | 40 | (intron 5 18078) | cctaccacaccatgcacgtg C/T acagccaagggttgttgact | 3400 | | |
| ABCA1 | 41 | (intron 5 18795) | ctgggctcttcctggacctg G/A ccagctaaaaggaaatctcc | 3401 | | |
| ABCA1 | 42 | (intron 5 18948) | gcattggtggtactaagaac G/A catattccctatcctatagg | 3402 | | |
| ABCA1 | 43 | (intron 5 19053) | ctcccccaacattaaaagtg T/C aagggatgcttattcaaatg | 3403 | | |
| ABCA1 | 44 | (intron 5 19148) | ggcccaagaaactgcatttt C/A gcatgctccctaaatgaagc | 3404 | | |
| ABCA1 | 45 | (intron 5 19229) | atgctaacagtgtagagtca C/T atgtgatgggaagcatcagg | 3405 | | |
| ABCA1 | 46 | (intron 5 19405) | cttgctcaatttattctgtc T/C atataactcaatattactga | 3406 | | |
| ABCA1 | 47 | (intron 5 19534) | catgtgaccctcttagctcc G/A cggattaactcctgtcctca | 3407 | | |
| ABCA1 | 48 | (coding region | gaaaccttctctgggttcct G/A tatcacaacctctctctccc | 3408 | | |
| | | 474 (Leu 158 Leu)) | | | | |
| ABCA1 | 49 | (intron 6 210) | gcaacctggcgtcatgggcc A/C gctggttaaaataaaattga | 3409 | | |
| ABCA1 | 50 | (intron 6 334) | acagttctgaggcaataacc G/A tggttaagggttattgatct | 3410 | | |
| ABCA1 | 51 | (intron 6 2288) | cttctttcaaagcttgtggt C/T cactggaccacgtatgaagt | 3411 | | |
| ABCA1 | 52 | (intron 6 2322) | atgaagtagaatagtttagg T/C ccagaaaggcaattaagtaa | 3412 | | |
| ABCA1 | 53 | (intron 6 2820) | gtgctttgatacattctgag T/G ttcagtaaagagacctgatg | 3413 | | |
| ABCA1 | 54 | (intron 7 416) | catcataaagatgacattgt G/A ggctgtcacagttggaaggc | 3414 | | |
| ABCA1 | 55 | (intron 7 471) | agaccacactatttagctta C/T ttagtaataacattgcaaag | 3415 | | |
| ABCA1 | 56 | (intron 7 504) | ttgcaaagaaaaattccgac G/A aagttttttcagcctaggaa | 3416 | | |
| ABCA1 | 57 | (intron 7 679) | gctctggtgaaattcctctc G/C ctaccccaaacatcatcatt | 3417 | | |
| ABCA1 | 58 | (intron 7 1740) | acaaatgctcaccctttcag C/T tggaatgattgaaattttgg | 3418 | | |
| ABCA1 | 59 | (intron 7 2122) | tgattaaggtggctactacc A/G ggtgctttctgcatatctcg | 3419 | | |
| ABCA1 | 60 | (intron 7 7753) | taggaattccaagctgtgaa T/C tttttactgaagctctttgg | 3420 | | |
| ABCA1 | 61 | (intron 7 8973) | atggaaatttgtttatattg A/T ctacagattgccaatattat | 3421 | | |
| ABCA1 | 62 | (intron 7 8976) | gaaatttgtttatattgact A/G cagattgccaatattattag | 3422 | | |
| ABCA1 | 63 | (intron 7 11327) | ctaacaatcttatttccatt G/C agtccttataaaagaagtgg | 3423 | | |
| ABCA1 | 64 | (intron 7 )11738 | ctgacgtttaagggagaccg C/T gtaggtccctttgaggactg | 3424 | | |
| ABCA1 | 65 | (intron 7 12295) | agtctgtaaattattgttct T/A ttttttctttagcttatgct | 3425 | | |
| ABCA1 | 66 | (intron 8 387) | tagcaaggccaatcatttta C/G caacacacatgcttgctaac | 3426 | | |
| ABCA1 | 67 | (intron 8 697) | ggaactgtctggtgtccccc A/T gcataggaagctgagccagg | 3427 | | |
| ABCA1 | 68 | (intron 8 3036) | ctttatgtgggaagaaattt T/G tttttttgattggggagtgg | 3428 | | |
| ABCA1 | 69 | (intron 8 3176) | aaatggcctggttctctgtc C/A cctttctgtctgtatgcctc | 3429 | | |
| ABCA1 | 70 | (intron 8 3364) | ggcagaaggcaaagcttagg A/T cctagagagtgctggaccac | 3430 | | |
| ABCA1 | 71 | (intron 8 3373) | caaagcttaggacctagaga G/A tgctggaccacgccactcac | 3431 | | |
| ABCA1 | 72 | (intron 8 3561) | cagggatttattaatgattt C/A ttgtgaaatgtttggaaata | 3432 | | |
| ABCA1 | 73 | (intron 8 3654) | agtgccggaatacatttgca T/C gtaagacagaacgctgcctg | 3433 | | |
| ABCA1 | 74 | (intron 8 4715) | ggcagaggggtctcagaatc C/T gcatttccaacaatgtctcc | 3434 | | |
| ABCA1 | 75 | (coding region | cgtattgtctgcgggcatcc C/T gagggaggggggctgaagat | 3435 | | |
| | | 936 (Pro 312 Pro)) | | | | |
| ABCA1 | 76 | (intron 9 2309) | cccctcaagagtcagtttaa A/G tgttggtcatgttagttgtc | 3436 | | |
| ABCA1 | 77 | (intron 9 2392) | atgggagggcttgtgcttca T/C gaaaacatttttccagatca | 3437 | | |
| ABCA1 | 78 | (intron 10 228) | tggggatggggaggactggc A/G cagggctgctgtgatggggt | 3438 | | |
| ABCA1 | 79 | (intron 10 319) | ttctgcggtccctggctccc C/T acctgactccaggtgaacaa | 3439 | | |
| ABCA1 | 80 | (intron 11 377) | gaaagaagtgtgggagcaaa A/C gcatgatgttacatgtagac | 3440 | | |
| ABCA1 | 81 | (intron 11 521) | agtgctctagagacaattgg G/A ttcaaatgtggagcaggctg | 3441 | | |
| ABCA1 | 82 | (intron 11 2850) | ctctatacaatcattatgct G/C ccattgaaataataaataca | 3442 | | |
| ABCA1 | 83 | (intron 11 2976) | ctccaattcggtagaaccag A/G gcttcatcttctctgtcgaa | 3443 | | |
| ABCA1 | 84 | (intron 11 3056) | gtttgcagctgctgtttttc C/T ggcagcacatctgtgcaggc | 3444 | | |
| ABCA1 | 85 | (intron 12 340) | ggcattatttgtgaaactta T/C ctaaaatcgaattcgggtcc | 3445 | | |
| ABCA1 | 86 | (intron 12 381) | aattaaatttttgaaatttt A/G tattaaaaattatattagta | 3446 | | |
| ABCA1 | 87 | (intron 14 1728) | caggctcagaggccttggcc C/T atcaccctggctcacgtgtg | 3447 | | |
| ABCA1 | 88 | (coding region | atgggcctggacaacagcat C/A ctctggtttagctggttcat | 3448 | | |
| | | 2040 (Ile 680 Ile)) | | | | |
| ABCA1 | 89 | (intron 15 1382) | cttttagacagaaaagttac G/A tgggatattatctcccacag | 3449 | | |
| ABCA1 | 90 | (intron 15 1453) | tatataaggagaaaccagtt G/A aaattacctattgaagaaac | 3450 | | |
| ABCA1 | 91 | (intron 15 1567) | ttctgcgtagttttgggtaa G/A tcacttatcttctttaggat | 3451 | | |
| ABCA1 | 92 | (intron 15 1617) | cagttgcctcatcagaaaga T/A gaacagcattacgcctctgc | 3452 | | |
| ABCA1 | 93 | (intron 16 95) | agttgagaacagaagatgat T/A gtcttttccaatgggacatg | 3453 | | |
| ABCA1 | 94 | (intron 16 452) | tggtgctttgcttgagtaat G/A ttttctgaactaagcacaac | 3454 | | |
| ABCA1 | 95 | (intron 16 657) | ctgttgcctcagtctgggct T/C cataggcatcagcagcccca | 3455 | | |
| ABCA1 | 96 | (intron 18 1730) | tgaaagttcaagcgcagtgc C/G ctgtgtccttacactccact | 3456 | | |
| ABCA1 | 97 | (intron 19 426) | aggaccttacagtgggtagt A/G tcaggaggggtcaggggctg | 3457 | | |
| ABCA1 | 98 | (intron 19 468) | aaagcaccagcgttagcctc A/G gtggcttccagcacgattcc | 3458 | | |
| ABCA1 | 99 | (intron 20 876) | ccctcctcatctaaagtgaa C/T acatggggctcatgtgcagg | 3459 | | |
| ABCA1 | 100 | (intron 22 118) | catgggatactcttctgtta T/G cacagaagagataaagggca | 3460 | | |
| ABCA1 | 101 | (intron 22 560) | aaagctttgccattctaggg G/A tcatagccatacagggtgaa | 3461 | | |
| ABCA1 | 102 | (intron 23 102) | accccttttgccatgttgaa A/G ccaccatctccctgctctgt | 3462 | | |
| ABCA1 | 103 | (intron 23 287) | gtcaaagaaaagagacttgt C/T aagaggtaagagccttggct | 3463 | | |
| ABCA1 | 104 | (intron 23 1063) | acctttcaccctcaggaagc G/A aggctgttcacacggcacac | 3464 | | |
| ABCA1 | 105 | (intron 25 321) | ctctttacttaagtacagtg T/G gaggaacagcggcatcagga | 3465 | | |
| ABCA1 | 106 | (intron 25 376) | gttagaaattcagcaacttg G/C gcccagctcagacctactga | 3466 | | |
| ABCA1 | 107 | (intron 25 478) | catacataggaaatgacaaa C/T gtttatggatggatagtcta | 3467 | | |
| ABCA1 | 108 | (intron 25 579) | tcatttaattctcaaaaaaa G/T atgaaaaaatgaacactcag | 3468 | | |
| ABCA1 | 109 | (intron 27 153) | aatggtaaaagccacttgtt C/T tttgcagcatcgtgcatgtg | 3469 | | |
| ABCA1 | 110 | (intron 28 1058) | actatcatgggagataatga C/T tatggttgtccatgattgga | 3470 | | |
| ABCA1 | 111 | (intron 28 1317) | caggacccagtgttctgagt C/T accctgaatgtgagcactat | 3471 | | |
| ABCA1 | 112 | (intron 30 372) | tatatgatttttaggttttg T/C ttatcagcttcttcgctttt | 3472 | | |
| ABCA1 | 113 | (intron 30 506) | ccttttaaaaagtaagcagt A/G gataaataaattcagtgaag | 3473 | | |
| ABCA1 | 114 | (intron 30 1033) | ctggatttcatggtgccttt G/C attttccacatgaaggttgt | 3474 | | |
| ABCA1 | 115 | (coding region | tcttccctttgcagagacac G/A ccctgccaggcaggggagga | 3475 | | |
| | | 4281 (Thr 1427 Thr)) | | | | |
| ABCA1 | 116 | (intron 33 626) | ggctccttgttactgatttc C/T gtcttttctctctgcctttt | 3476 | | |
| ABCA1 | 117 | (intron 33 719) | taatagccctcatgctagaa G/A ggagccggagcctgtgtata | 3477 | | |
| ABCA1 | 118 | (intron 33 726) | cctcatgctagaagggagcc G/A gagcctgtgtataaggccag | 3478 | | |
| ABCA1 | 119 | (intron 33 889) | ctttcctcaatgtctcagct A/G tctaactgtgtgtgtaatca | 3479 | | |
| ABCA1 | 120 | (intron 33 1097) | ctgtgcaccccactgtctgg G/C ttttaatgtcaggctgttct | 3480 | | |
| ABCA1 | 121 | (intron 35 234) | aacctatctaaacctcagtt T/C cctcatctgtgaaatggaga | 3481 | | |
| ABCA1 | 122 | (intron 37 411) | aactctgtacattttatcag C/T agcttatccatccattgcaa | 3482 | | |
| ABCA1 | 123 | (intron 37 1224) | caggcataggtgattcagag A/G tgaaaggtcaagtccctgaa | 3483 | | |
| ABCA1 | 124 | (intron 37 1720) | aaattaaaattactctgact G/T ggaatccatcgttcagtaag | 3484 | | |
| ABCA1 | 125 | (intron 40 251) | tgaaggtaaggaaaatagtg T/G tatttgcttggatccactgg | 3485 | | |
| ABCA1 | 126 | (intron 40 252) | gaaggtaaggaaaatagtgt T/C atttgcttggatccactggc | 3486 | | |
| ABCA1 | 127 | (intron 40 319) | agcactggaaaagtcaaacc A/G taactttgagaattaggtga | 3487 | | |
| ABCA1 | 128 | (intron 40 957) | cttgttactcttttttcctt G/C tcatgggtgatagccatttg | 3488 | | |
| ABCA1 | 129 | (intron 41 146) | tgatgtgggcatcccgcagc C/T ccctccctgcccatcctgga | 3489 | | |
| ABCA1 | 130 | (intron 42 239) | catttggtttatatgcttac A/C tttatgtgttagttattaaa | 3490 | | |
| ABCA1 | 131 | (intron 42 321) | aataaatggttgattttgag T/A ttgagtttcatagtccaaaa | 3491 | | |
| ABCA1 | 132 | (intron 42 322) | ataaatggttgattttgagt T/C tgagtttcatagtccaaaaa | 3492 | | |
| ABCA1 | 133 | (intron 42 533) | agatgaaaaattatgtagat G/A ataatgaatgatacggttct | 3493 | | |
| ABCA1 | 134 | (intron 42 546) | tgtagatgataatgaatgat A/G cggttctaaaaagacaggtt | 3494 | | |
| ABCA1 | 135 | (intron 43 739) | tacagccacacttaaaatgg T/A cccattatgaaatacatatt | 3495 | | |
| ABCA1 | 136 | (intron 44 18) | taggtgagaaaagaagtggc T/C tgtattttgctgcaaagact | 3496 | | |
| ABCA1 | 137 | (intron 44 264) | acaatataatttgcttgttt T/C ttaagagtataatttagtga | 3497 | | |
| ABCA1 | 138 | (intron 44 279) | tgttttttaagagtataatt T/C agtgatttttggtaaattga | 3498 | | |
| ABCA1 | 139 | (intron 44 508) | tttacattgctacataaaat C/T cccctatgtacatgtaccta | 3499 | | |
| ABCA1 | 140 | (intron 44 1477) | gatctcctctcctgtctctt A/T catttttgcagtagcaatgt | 3500 | | |
| ABCA1 | 141 | (intron 44 1665) | tggttgtaagaactgatttg G/A ttggtatagctgtgagggcc | 3501 | | |
| ABCA1 | 142 | (intron 44 1956) | gtgttgctcacactcaaaat T/G tctgggccttctcatttggt | 3502 | | |
| ABCA1 | 143 | (intron 45 68) | aatatataccttatggcttt T/C ccacacgcattgacttcagg | 3503 | | |
| ABCA1 | 144 | (intron 46 608) | ttatactgacttcaatagag G/C tttcagacaaaaagttgttt | 3504 | | |
| ABCA1 | 145 | (intron 47 336) | ttcacaattgtaaacaccac T/C acactgaacagcatcatccc | 3505 | | |
| ABCA1 | 146 | (3′untranslated region | aacaaaaatgtgggtgtctc C/T aggcacgggaaacttggttc | 3506 | | |
| | | 7479) | | | | |
| ABCA1 | 147 | (3′untranslated region | aggagcccactgtaacaata C/T tgggcagccttttttttttt | 3507 | | |
| | | 8226) | | | | |
| ABCA1 | 148 | (3′untranslated region | ttccagaatttgaatattaa C/T gctaaaggtgtaagacttca | 3508 | | |
| | | 8697) | | | | |
| ABCA1 | 149 | (3′untranslated region | aactattttgaagaaaacac A/G acattttaatacagattgaa | 3509 | | |
| | | 9097) | | | | |
| ABCA1 | 150 | (5′flanking region | tgacttaaatatttagacat (AT) acattttaatacagattgaa | 3510 | | |
| | | (−1033)− (−1032)) | | | | |
| ABCA1 | 150 | (5′flanking region | tgacttaaatatttagacat ggtgtgtaggcctgcattcc | 3511 | | |
| | | (−1033)− (−1032)) | | | | |
| ABCA1 | 151 | (intron 5 6368) | ttctgatggggttgttgctg C/Δ tgagaatcatgactgggtgg | 3512 | | |
| ABCA1 | 152 | (intron 5 9709) | cattttctgtctgaaccccc T/Δ cacccattcaggcagctgct | 3513 | | |
| ABCA1 | 153 | (intron 5 13816) | tccctacttctccttttttt T/Δ catttgcctcctccacccac | 3514 | | |
| ABCA1 | 154 | (intron 10 270 − 271) | cttttcagggaggagccaaa (G) cgctcattgtctgtgcttct | 3515 | | |
| ABCA1 | 154 | (intron 10 270 − 271) | cttttcagggaggagccaaa cgctcattgtctgtgcttct | 3516 | | |
| ABCA1 | 155 | (intron 20 611 − 612) | tttagcccatcctctccccc (C) gccaccctccttattgaggc | 3517 | | |
| ABCA1 | 155 | (intron 20 611 − 612) | tttagcccatcctctccccc gccaccctccttattgaggc | 3518 | | |
| ABCA1 | 156 | (intron 32 391 − 392) | gagtgccttgggtactctct (T) gatgggggactccatgataa | 3519 | | |
| ABCA1 | 156 | (intron 32 391 − 392) | gagtgccttgggtactctct gatgggggactccatgataa | 3520 | | |
| ABCA1 | 157 | (intron 37 847) | gctgtatattgtgaatgtcc C/Δ gttttcaaaagcaaagccaa | 3521 | | |
| ABCA4 | 1 | (5′flanking region | tgccatcataagcagaaact A/C tctctctcttcttggaagct | 3522 | | |
| | | −1005) | | | | |
| ABCA4 | 2 | (5′flanking region | gtctagagtctttcaaagag A/T acacattctgagatttgagg | 3523 | | |
| | | −819) | | | | |
| ABCA4 | 3 | (5′flanking region | agcaccaccccattgcaggg C/A tggaatgacagtaatgggcc | 3524 | | |
| | | −680) | | | | |
| ABCA4 | 4 | (intron 1 208) | tgcccttcccaggaagatgt G/A tttctctgtcctcagccaca | 3525 | | |
| ABCA4 | 5 | (intron 1 234) | ctgtcctcagccacatgaaa A/G tcttttgcctaccgtgcctg | 3526 | | |
| ABCA4 | 6 | (intron 1 510) | agctcacgatcaagtcacag T/C ttaactggacacattatttt | 3527 | | |
| ABCA4 | 7 | (intron 1 1527) | gcttaacaaccagcataaaa G/A agagcagcatgggacacgct | 3528 | | |
| ABCA4 | 8 | (intron 1 2077) | caggactgtagctgctggcc T/C aaaatgagcccattcctgtg | 3529 | | |
| ABCA4 | 9 | (intron 1 2174) | ccctctcaatctggcctttc G/C ctggcatgggtgggcgactc | 3530 | | |
| ABCA4 | 10 | (intron 1 2246) | gctcccagggagatggagcc A/G ctcgggctgagggccttggc | 3531 | | |
| ABCA4 | 11 | (intron 1 2364) | ttctgtctggcacgcctccc G/A atggctccccacctgctacc | 3532 | | |
| ABCA4 | 12 | (intron 1 4243) | ctccctggggtatgcctgta C/G gcagttaagcgtcaaggaca | 3533 | | |
| ABCA4 | 13 | (intron 1 4287) | atgccgctctggggagggga A/C gctgagcatgattttggaag | 3534 | | |
| ABCA4 | 14 | (intron 1 4309) | ctgagcatgattttggaagc C/T ggcagaagaggctattgtga | 3535 | | |
| ABCA4 | 15 | (intron 1 4416) | tgcagcaaccgcccccgccc C/T ccgccaaaaacaaacacact | 3536 | | |
| ABCA4 | 16 | (intron 1 4996) | tttacccctggaacaggcag G/A ccaagctggc t/c ggtcccctc | 3537 | | |
| ABCA4 | 17 | (intron 1 5007) | aacaggcag g/a ccaagctggc T/C ggtcccctccctgatacaca | 3538 | | |
| ABCA4 | 18 | (intron 1 5080) | gtgtgtggctggtttcttag C/G aagcaccatggttccaagtt | 3539 | | |
| ABCA4 | 19 | (intron 1 5152) | gggagatgaacgtaagtgga G/A ggcaggcctacaaggttgca | 3540 | | |
| ABCA4 | 20 | (intron 1 7110) | ccactggatctgcttttgga A/G tcaagagtccttaagctcca | 3541 | | |
| ABCA4 | 21 | (intron 1 7290) | gatttttgttggctttgcaa T/A ggatcacagtcatttattca | 3542 | | |
| ABCA4 | 22 | (intron 1 7483) | tctgagcctctttccttaac T/C gcagagtgagtgg c/t tacaga | 3543 | | |
| ABCA4 | 23 | (intron 1 7497) | cttaac t/c gcagagtgagtgg C/T tacagagaaatctttactac | 3544 | | |
| ABCA4 | 24 | (intron 2 1067) | tcaagcagcagcagcaactg C/A gtggagtcttcttgaactaa | 3545 | | |
| ABCA4 | 25 | (intron 2 1243) | cacccagcacagggactggc A/T cacatgagatgctcctgctt | 3546 | | |
| ABCA4 | 26 | (intron 3 26) | tgttgagatccctaccatgc A/G ggggaggaagttgcacaccc | 3547 | | |
| ABCA4 | 27 | (intron 3 101) | agcatggagcactgagtgtt C/T ttgtggctttgctgagcccc | 3548 | | |
| ABCA4 | 28 | (intron 3 330) | tgcttgggtggagtgaatca T/C tgtaggagaaaaactcagtt | 3549 | | |
| ABCA4 | 29 | (intron 3 470) | tgaagtcaggtttacaaagt C/G aagtttacttcttgggagaa | 3550 | | |
| ABCA4 | 30 | (intron 3 634) | tgaaaaccaatgacccctct T/C ccaagaaaaatggccacata | 3551 | | |
| ABCA4 | 31 | (intron 3 1016) | ccttgggggagctcagtatg A/G ttcttccaggagaagcctgc | 3552 | | |
| ABCA4 | 32 | (intron 3 1554) | gaaagttgggtttcatgttt T/C ccaagaaaaatggccacata | 3553 | | |
| ABCA4 | 33 | (intron 3 1686) | ctagacattctcacagagcc A/G agggcagcaaggcggggctc | 3554 | | |
| ABCA4 | 34 | (intron 3 1823) | ttcacctctctccatggacc A/G agggcagcaaggcggggctc | 3555 | | |
| ABCA4 | 35 | (intron 3 1938) | caaattcctgggaacaaatc G/A ggttgacccagc t/g ttattct | 3556 | | |
| ABCA4 | 36 | (intron 3 1951) | acaaatc g/a ggttgacccagc T/G ttattctccctgtcccatca | 3557 | | |
| ABCA4 | 37 | (intron 3 2063) | ggctgtcagagcctacctgc G/T tgaatgggtggaagg g/a cagg | 3558 | | |
| ABCA4 | 38 | (intron 3 2079) | ctgc t/g tgaatgggtggaagg G/A caggtctcagagaattgggt | 3559 | | |
| ABCA4 | 39 | (intron 3 2186) | agacacacagagcatgggac C/T gagaggcgagcagaccctgc | 3560 | | |
| ABCA4 | 40 | (intron 3 2214) | gagcagaccctgccaaaact G/A ggagactgaatagatcgctc | 3561 | | |
| ABCA4 | 41 | (intron 4 3182) | cccccagagccacagcagcc C/G tgtctcctgggtggtcttgt | 3562 | | |
| ABCA4 | 42 | (intron 4 3515) | agtataataaaagcaggagc C/T atagcccccaactctcaaga | 3563 | | |
| ABCA4 | 43 | (intron 4 3952) | agagaagccactgtgccact G/C tgtggtcgaacttcaagacc | 3564 | | |
| ABCA4 | 44 | (intron 4 4637) | aatcacttgccccaaggtca C/T cttaactgttaggtgttctt | 3565 | | |
| ABCA4 | 45 | (intron 4 5319) | acctctaggggctcccagag A/G ccccaagaacagaaccttcc | 3566 | | |
| ABCA4 | 46 | (intron 6 2266) | cacccttgcagacctcagac G/A ggtcctgggggcttgctttc | 3567 | | |
| ABCA4 | 47 | (intron 6 2857) | ccagaggagaaagctctgcc G/A tag t/c cggcctcagttaacca | 3568 | | |
| ABCA4 | 48 | (intron 6 2861) | aggagaaagctctgcc g/a tag T/C cggcctcagttaaccacgga | 3569 | | |
| ABCA4 | 49 | (intron 6 3078) | gcaggcattaaaatgggact T/G tgcctttattgctcctgggc | 3570 | | |
| ABCA4 | 50 | (intron 6 3375) | ttaaatgccaaatgagttct C/G attaacaaagaaagagggaa | 3571 | | |
| ABCA4 | 51 | (intron 6 3412) | ggaaaatctcagtaaaccac C/T gtgacggcatctacccactt | 3572 | | |
| ABCA4 | 52 | (intron 6 4635) | ctttcgggtggatattgcta C/T gtcaagtgtctgggaaagcc | 3573 | | |
| ABCA4 | 53 | (intron 6 −264) | aaacagcaattagaatcact T/C tgaaatagtgatagtattta | 3574 | | |
| ABCA4 | 54 | (intron 7 828) | gatgtgggaaagttagagaa G/C agcccattgtactaatgctc | 3575 | | |
| ABCA4 | 55 | (intron 7 1019) | aggcttcttgactgtctaga T/C agcaagtctaatcatttgtg | 3576 | | |
| ABCA4 | 56 | (intron 8 374) | gtaaacacggctgtgggatg C/A ttttacaaacacaatatcgt | 3577 | | |
| ABCA4 | 57 | (intron 8 874) | tgatgagcttgttattggtg G/A ggtacagcctattaatttag | 3578 | | |
| ABCA4 | 58 | (intron 9 605) | tcgtgtctctgtcttgatct C/T tgtctggttttaggccaact | 3579 | | |
| ABCA4 | 59 | (coding region 1268 | aacttttgaagaactggaac G/A c/t gttaggaagttggtcaaag | 3580 | | |
| | | (Arg 423 Arg or His 423 | | | | |
| | | His)) | | | | |
| ABCA4 | 60 | (coding region 1269 | acttttgaagaactggaac g/a C/T gttaggaagttggtcaaagc | 3581 | | |
| | | (Arg 423 Arg or His 423 | | | | |
| | | His)) | | | | |
| ABCA4 | 61 | (intron 11 5687) | atcatgtaatgtactttaga C/G tcagatatataaatatttgt | 3582 | | |
| ABCA4 | 62 | (intron 11 7136) | gacttcccaacttaccttag T/C ggagctgtagtcacatagaa | 3583 | | |
| ABCA4 | 63 | (intron 11 7180) | acgctcataaatgcttctct G/A ggctgtaaaggttgaatttt | 3584 | | |
| ABCA4 | 64 | (intron 11 7701) | gttagacgcaggcattacct C/T gtggctttgccccagtgtga | 3585 | | |
| ABCA4 | 65 | (intron 11 8073) | gggatgtttgcccacatcca T/C tggcatttctcaaaaggaac | 3586 | | |
| ABCA4 | 66 | (intron 11 8586) | cagctgcctgcgctggagag G/A gctcaaacctcttccgccag | 3587 | | |
| ABCA4 | 67 | (intron 11 11234) | cccaaataattttgtttttc G/A ttttaggaattaaatttcag | 3588 | | |
| ABCA4 | 68 | (intron 11 11641) | aagaaacaaacatttattga C/G aacttttggtgtgtgacctg | 3589 | | |
| ABCA4 | 69 | (intron 11 11808) | tggtatttcttaaagaaata C/T caattccatttccttttaac | 3590 | | |
| ABCA4 | 70 | (intron 11 11923) | aagatcattattaatatctc A/G tcagcgtggtgtcacttaag | 3591 | | |
| ABCA4 | 71 | (intron 12 305) | tcaccctgtggtcgggaggt G/A tgagtgagctatccaagccc | 3592 | | |
| ABCA4 | 72 | (intron 13 1461) | ttgggtttcagtgtcagcat G/A tagctgtctactcagatccc | 3593 | | |
| ABCA4 | 73 | (intron 14 1268) | ggagctgagccccttgtcct T/C atctaggtttcccttgttct | 3594 | | |
| ABCA4 | 74 | (intron 17 23) | gagtcctttaaaacacaaat C/G ttaatgtttgaaatcaactc | 3595 | | |
| ABCA4 | 75 | (intron 17 715) | gggactcccctagagctgaa G/A tactctcccatctgtttgtt | 3596 | | |
| ABCA4 | 76 | (intron 18 1282) | ggaagatgaagaacctaagc C/T gcttccagaaattcatgagg | 3597 | | |
| ABCA4 | 77 | (intron 20 − 195) | acagattattccattgtatg C/A atgaactatgtaagccatcc | 3598 | | |
| ABCA4 | 78 | (intron 23 755) | ctggctgccgctggggtttc C/T tatgtccatccacggggagg | 3599 | | |
| ABCA4 | 79 | (intron 26 702) | tatcaaatacaactcagacg T/G cagtctcctggcccctttga | 3600 | | |
| ABCA4 | 80 | (intron 27 156) | cctgctttccaaacccttat C/T ttgattcttggtaacatgaa | 3601 | | |
| ABCA4 | 81 | (intron 27 385) | tttaaagaacagtgagtcac G/A tgacttgctctttgaaatgc | 3602 | | |
| ABCA4 | 82 | (intron 28 299) | gacatgccatcagaccactg C/T gagtgttcaggcagcctacc | 3603 | | |
| ABCA4 | 83 | (intron 29 168) | ctccttccacacttgtgtgc A/G gggacattcactacctccta | 3604 | | |
| ABCA4 | 84 | (intron 29 497) | gctgtcaataaggaccaaaa C/T agactaatttcaaattcctc | 3605 | | |
| ABCA4 | 85 | (intron 29 567) | agctgctaggaataaaaagg G/A agacaaaac g/a atccacaagc | 3606 | | |
| ABCA4 | 86 | (intron 29 577) | aataaaaagg g/a agacaaaac G/A atccacaagctagagatggt | 3607 | | |
| ABCA4 | 87 | (intron 30 − 2494) | aatcagagctcatctgctgc A/G tcatagggatcccaaaagaa | 3608 | | |
| ABCA4 | 88 | (intron 30 − 2169) | aatgtaacagccaaagtcct A/G gaaaaaggcaagccagttcc | 3609 | | |
| ABCA4 | 89 | (intron 31 535) | ctaactgtgaattatcatct T/G tgatcactgccctttgagat | 3610 | | |
| ABCA4 | 90 | (intron 35 209) | tctccccaacatttatgtgg C/A aagtaagtttacatttggtt | 3611 | | |
| ABCA4 | 91 | (intron 37 525) | taaatttgaatgagtaattc A/G tccatctcggcctcagtttc | 3612 | | |
| ABCA4 | 92 | (intron 37 766) | tgttgcaggctggagaaccc T/G cctatgaattgtacagggct | 3613 | | |
| ABCA4 | 93 | (intron 37 856) | aaaaccccatgaagtggtca A/G ggcaggcatcattatctcca | 3614 | | |
| ABCA4 | 94 | (intron 38 62) | tagtagagtatgtgttggtc G/A agcagagccaggggcaagca | 3615 | | |
| ABCA4 | 95 | (intron 38 761) | tccttgggcaagttaatctt G/A atgaagagactgggtgttct | 3616 | | |
| ABCA4 | 96 | (intron 38 1315) | cagagtcagactctggaaag G/T c/a ggggggataagaacacagc | 3617 | | |
| ABCA4 | 97 | (intron 38 1316) | agagtcagactctggaaag g/t C/A ggggggataagaacacagcc | 3618 | | |
| ABCA4 | 98 | (intron 38 1561) | gtattttcatgtaaattatc C/A g/a atacacagctgctatggaa | 3619 | | |
| ABCA4 | 99 | (intron 38 1562) | tattttcatgtaaattatc c/a G/A atacacagctgctatggaaa | 3620 | | |
| ABCA4 | 100 | (intron 38 2874) | ctagacaaagggg a/c agctcc C/T gcccactagaaacttgcagg | 3621 | | |
| ABCA4 | 101 | (intron 40 1904) | gacactgtacagccagccca A/C tcctgaccccttttcttcat | 3622 | | |
| ABCA4 | 102 | (coding region 5814 | ggaaataaaactgacatctt A/G aggctacatgaactaaccaa | 3623 | | |
| | | (Leu 1938 Leu)) | | | | |
| ABCA4 | 103 | (intron 41 122) | ggaaataaaactgacatctt A/G aggctacatgaactaaccaa | 3624 | | |
| ABCA4 | 104 | (intron 41 411) | atttggttcccagttttatg T/G agggtcatcatccctgtgtt | 3625 | | |
| ABCA4 | 105 | (intron 41 443) | gttctcagtccggtttcttc G/A tatcttgcagatttatcc a/g g | 3626 | | |
| ABCA4 | 106 | (coding region 5844 | c g/a tatcttgcagatttatcc A/G ggcacctccagcccagcagt | 3627 | | |
| | | (Pro 1948 Pro)) | | | | |
| ABCA4 | 107 | (intron 43 328) | tttgtagcctattcctataa A/G aatgcaccattgcttc c/g cat | 3628 | | |
| ABCA4 | 108 | (intron 43 345) | taa a/g aatgcaccattgcttc C/G cattacctccctccacacat | 3629 | | |
| ABCA4 | 109 | (intron 43 370) | acctccctccacacattttt A/G caaaa c/t gtttcagggagttt | 3630 | | |
| ABCA4 | 110 | (intron 43 376) | ctccacacattttt a/g caaaa C/T gtttcagggagtttactgag | 3631 | | |
| ABCA4 | 111 | (intron 43 670) | ttaaacagactggtccccta T/C gggcaggacagagaggatga | 3632 | | |
| ABCA4 | 112 | (intron 43 822) | gttaggtgctgctgacatct G/A tccagcatctgcttgactgg | 3633 | | |
| ABCA4 | 113 | (intron 43 915) | ggcaggacgagtcctgagca C/T gcttcactggctcagacagg | 3634 | | |
| ABCA4 | 114 | (intron 43 1242) | actgagctggacgctagaaa G/T aaactataggcttaagacac | 3635 | | |
| ABCA4 | 115 | (intron 43 1671) | tagagaagtttacttccatc G/A ggacacatgcatcttttcta | 3636 | | |
| ABCA4 | 116 | (intron 43 2036) | ttgaaggatactcagtaatt G/A ctttttttcttgcagtattt | 3637 | | |
| ABCA4 | 117 | (intron 45 176) | gtgtttggttcacacagctc C/T ggagaaaaacaagtca c/t ggc | 3638 | | |
| ABCA4 | 118 | (intron 45 193) | ctc c/t ggagaaaaacaagtca C/T ggcacagccttgacttggga | 3639 | | |
| ABCA4 | 119 | (intron 47 238) | cccaagtctctggatggggc A/G tctgatcaggatgcatgcag | 3640 | | |
| ABCA4 | 120 | (intron 47 269) | atgcatgcagagcctggctg G/A gatgagggagggctgctacc | 3641 | | |
| ABCA4 | 121 | (intron 47 326) | accacttatctcaacagatc C/G gggacctgtggcctatttac | 3642 | | |
| ABCA4 | 122 | (intron 47 715) | aagtcactaagctggttggt G/A ggaggaacagcacataac c/t c | 3643 | | |
| ABCA4 | 123 | (intron 47 734) | t g/a ggaggaacagcacataac C/T caccttatctatgctgaggt | 3644 | | |
| ABCA4 | 124 | (intron 47 931) | ggacactgcatagatatcta T/C agaaatagcagcatgtcagg | 3645 | | |
| ABCA4 | 125 | (intron 47 1260) | acactctctggtggaccatc A/C ctcatccaagagagggtaac | 3646 | | |
| ABCA4 | 126 | (intron 48 1663) | tctcgctcttctcttacctc T/C aggtgtttgtaaattttgct | 3647 | | |
| ABCA4 | 127 | (intron 49 127) | agagagccccacccacacca C/T ggtccctaccaagtccccac | 3648 | | |
| ABCA4 | 128 | (5′flanking region | gtaaatctcagttgaatcag (TCA)14-16 atttttcagtctggttcct | 3649 | | |
| | | (−1441)-(−1400)) | | | | |
| ABCA4 | 129 | (intron 1 4712-4720) | gaggggcggggactataggc (A)8-10 cagcctaattcaaggatgag | 3650 | | |
| ABCA4 | 130 | (intron 1 7295-7304) | ttgttggctttgcaa t/a ggat CACAGTCAT/Δ ttattcactcatt | 3651 | | |
| ABCA4 | 131 | (intron 2 951-952) | cctgtccatcagactcttct TT/Δ acctctccccgaggagccca | 3652 | | |
| ABCA4 | 132 | (intron 3 2642-2653) | cctgggtgacagagcgagat (A)10-12 tagcatgagatattattact | 3653 | | |
| ABCA4 | 133 | (intron 4 5202) | cacaaagcatctgacacccc C/Δ atccagccctggctaacttt | 3654 | | |
| ABCA4 | 134 | (intron 6 3029-3044) | cactaaaaacaaaaatttac (A)16-18 cctgaaagaaattgcaggca | 3655 | | |
| ABCA4 | 135 | (intron 6 5138-5139) | ttcatgacagatcagatgtt (G) cttttatggatttacaaaga | 3656 | | |
| ABCA4 | 135 | (intron 6 5138-5139) | ttcatgacagatcagatgtt cttttatggatttacaaaga | 3657 | | |
| ABCA4 | 136 | (intron 6 5985) | tttccttcttcaaacccccc C/Δ agactaggagaaggtctgtc | 3658 | | |
| ABCA4 | 137 | (intron 6 6094) | gggacggacagaaaaagacc T/Δ agtttctgttgagccaaaga | 3659 | | |
| ABCA4 | 138 | (intron 6 − 161) | tattttttcaattaaataaa A/Δ gagttttttgtttctaaaag | 3660 | | |
| ABCA4 | 139 | (intron 7 809-810) | gggccgagtatgcacactga (TG) tgtgggaaagttagagaa g/c a | 3661 | | |
| ABCA4 | 139 | (intron 7 809-810) | tattttttcaattaaataaa tgtgggaaagttagagaa g/ | 3662 | | |
| ABCA4 | 140 | (intron 8 472-484) | atcttccccacctttcacta (T)10-13 ggtcttctatggggtaaagg | 3663 | | |
| ABCA4 | 141 | (intron 9 48-71) | gtaccctggacctcccagaa (GT)11-13 gagagagatgtgccttcctg | 3664 | | |
| ABCA4 | 142 | (intron 9 554) | ataggggcagaaaagacaca A/Δ ccaaaagttctctctcactt | 3665 | | |
| ABCA4 | 143 | (intron 10 11) | catgatcagagtaagggggg G/Δ ttggaggatggggaggggag | 3666 | | |
| ABCA4 | 144 | (intron 11 4242) | ggagaggaaatgatgttagt G/Δ cctcctgtaaataggcccag | 3667 | | |
| ABCA4 | 145 | (intron 11 13743-13753) | tgctcttttgtgggtaatgg (T)9-11 cctcttccaggagaagaaaa | 3668 | | |
| ABCA4 | 146 | (intron 13 636-637) | cggggtggagggttgggagg (G) ctcatttgtcattatagatg | 3669 | | |
| ABCA4 | 146 | (intron 13 636-637) | cggggtggagggttgggagg ctcatttgtcattatagatg | 3670 | | |
| ABCA4 | 147 | (intron 18 569-570) | tgctgccctcatcttctctc TT/Δ aaactagttctgtatttctc | 3671 | | |
| ABCA4 | 148 | (intron 20 (− 304)-(-297) | tataacctgacttttttttc (A)7-9 ggattgcttttttaaacata | 3672 | | |
| ABCA4 | 149 | (intron 22 1236-1246) | gctgaattagttcccttggg (T)9-11 agttaactcctgatttttgc | 3673 | | |
| ABCA4 | 150 | (intron 26 4326-4635) | gataatcaatgctgtaaggg (A)9-10 tggcattagagatccagacc | 3674 | | |
| ABCA4 | 151 | (intron 33 115-116) | taaaaccgtcttgtttgttt GT/Δ ttacatggtttttagggccc | 3675 | | |
| ABCA4 | 152 | (intron 36 1078) | taagcagctatcacttaaca A/Δ tacaaaaccagagattatca | 3676 | | |
| ABCA4 | 153 | (intron 37 290-291) | ccttgaccaaagcctggggg (T) cagccattcccca a/g cccctc | 3677 | | |
| ABCA4 | 153 | (intron 37 290-291) | ccttgaccaaagcctggggg cagccattcccca a/g cccctc | 3678 | | |
| ABCA4 | 154 | (intron 38 896) | ataaaaagagggggaaaaaa A/Δ gaaggcagtcgctgcagggc | 3679 | | |
| ABCA4 | 155 | (intron 38 1209-1210) | gtggacccctgagactgact CT/Δ ttccagatcttgttagggtt | 3680 | | |
| ABCA4 | 156 | (intron 38 1322) | agactctggaaag g/t c/a ggggg G/Δ ataagaacacagccccag | 3681 | | |
| ABCA4 | 157 | (intron 38 3107) | gggccccacctgctgaagag A/Δ gggggggtggggtttgcccc | 3682 | | |
| ABCA4 | 158 | (intron 40 152) | ttttctccaataatacaagt CT/Δ ttccagatcttgttagggtt | 3683 | | |
| ABCA4 | 159 | (intron 43 330) | tgtagcctattcctataa a/g a A/Δ tgcaccattgcttc c/g ca | 3684 | | |
| ABCA4 | 160 | (intron 43 1354) | tttaattggcccagccatgc C/Δ tttggtggcttttgtcattg | 3685 | | |
| ABCA4 | 161 | (intron 47 1305-1308) | catcctgctgaaggagaaag AAAG/Δ caccaatggcccaagcccta | 3686 | | |
| ABCA7 | 1 | (5′flanking region − 1596) | agaatgttggccccctcccc C/T t c/t ctgcatcctctgcagaag | 3687 | | |
| ABCA7 | 2 | (5′flanking region − 1594) | aatgttggccccctcccc c/t t C/T ctgcatcctctgcagaagcc | 3688 | | |
| ABCA7 | 3 | (5′flanking region − 1180) | ggccagtgagtgacgggcag G/A tcgcccaaatagcagcgtgc | 3689 | | |
| ABCA7 | 4 | (5′flanking region − 460) | agagctggggtcgtgcctcc A/G gctgggcaactgcctgtctc | 3690 | | |
| ABCA7 | 5 | (5′untranslated region | ctctgtcccgtcccctgccc A/G gtctcaccatggccttctgg | 3691 | | |
| ABCA7 | 6 | (intron 5 91) | ccccgggccaaggacctccc G/A ttccaggcatccaggctgtc | 3692 | | |
| ABCA7 | 7 | (coding region 563 | cagcttgttggaggccgctg A/G ggacctggcccaggaggtac | 3693 | | |
| | | (Glu 188 Gly)) | | | | |
| ABCA7 | 8 | (intron 8 103) | gccggagggtcacggaaact A/G tttgaagaagtaggagttag | 3694 | | |
| ABCA7 | 9 | (intron 8 166) | tgcggaggatcagaggcaca C/T gcaggagcaaggcagagggg | 3695 | | |
| ABCA7 | 10 | (coding region 955 | accggaccttcgaggagctc A/G ccctgctgagggatgtccgg | 3696 | | |
| | | (Thr 319 Ala)) | | | | |
| ABCA7 | 11 | (intron 9 421) | tttttttttttttttttttt T/A taagagatggagtctcactc | 3697 | | |
| ABCA7 | 12 | (intron 9 463) | gttgcccaggctggactgca G/A tgg c/t gagatcttggctcact | 3698 | | |
| ABCA7 | 13 | (intron 9 467) | cccaggctggactgca g/a tgg C/T gagatcttggctcactgcaa | 3699 | | |
| ABCA7 | 14 | (intron 9 488) | gagatcttggctcactgcaa C/T ctccgcctcctggattcaag | 3700 | | |
| ABCA7 | 15 | (coding region 1184 | cgcacacgctgatgtggggc A/G cctggtgggcacgctgggcc | 3701 | | |
| | | (His 395 Arg)) | | | | |
| ABCA7 | 16 | (intron 10 10) | gagtgacggaggtgagggcc T/C gtccacctgcggggtctgtt | 3702 | | |
| ABCA7 | 17 | (coding region 1824 | cctgggccccggccacgtgc G/A catcaaaatccgcatggaca | 3703 | | |
| | | (Arg 463 His)) | | | | |
| ABCA7 | 18 | (intron 13 55) | ggtgcgctggagggtgacag A/G caggggcggccccacgtggg | 3704 | | |
| ABCA7 | 19 | (coding region 1824 | cccttcctgctcagcgccgc A/G ctgctggttctggtgctcaa | 3705 | | |
| | | (Ala 608 Ala)) | | | | |
| ABCA7 | 20 | (intron 13 55) | ggtgcgctggagggtgacag A/G caggggcggccccacgtggg | 3706 | | |
| ABCA7 | 21 | (intron 13 78) | ggggcggccccacgtgggtg C/A gcgcccccaggccaatccag | 3707 | | |
| ABCA7 | 22 | (coding region 1851 | cgttgcctctcacagctggg A/G gacatcctcccctacagcca | 3708 | | |
| | | (Gly 617 Gly)) | | | | |
| ABCA7 | 23 | (coding region 2153 | cgagggcgcgcagtggcaca A/C cgtgggcacccggcctacgg | 3709 | | |
| | | (Asn 718 Thr)) | | | | |
| ABCA7 | 24 | (intron 15 34) | ggcggggctccgggccgggt C/G gcacctgctttgcgggaggc | 3710 | | |
| ABCA7 | 25 | (intron 16 8) | ctggacccaaagggtgaggc A/C ctacgaggcttaatagctgg | 3711 | | |
| ABCA7 | 26 | (intron 16 161) | tcccgcagcttttataggcc C/T cggcccagcaggtcccggat | 3712 | | |
| ABCA7 | 27 | (coding region 2385 | caccccatctctgcagtgct G/A gtagaagaggcaccgcccgg | 3713 | | |
| | | (Leu 795 Leu)) | | | | |
| ABCA7 | 28 | (coding region 2421 | cccggcctgagtcctggcgt C/A tccgttcgcagcctggagaa | 3714 | | |
| | | (Val 807 Val)) | | | | |
| ABCA7 | 29 | (intron 20 166) | cgagacagtaagagttgggg A/G tagacagaggttcccctgga | 3715 | | |
| ABCA7 | 30 | (coding region 3027 | ctgctgggagaccgtgtggc C/T gtggtggcaggtggccgctt | 3716 | | |
| | | (Ala 1009 Ala)) | | | | |
| ABCA7 | 31 | (intron 22 1386) | gggtggggcgtgagccgggg C/T tccctgaagcacccccttgt | 3717 | | |
| ABCA7 | 32 | (coding region 3417 | gggtggggcgtgagccgggg C/T tccctgaagcacccctttgt | 3718 | | |
| | | (Leu 1139 Leu)) | | | | |
| ABCA7 | 33 | (intron 23 147) | ggagctctggtggctcagat G/A tcccttgggaaggcctgggg | 3719 | | |
| ABCA7 | 34 | (coding region 3528 | gctggcctagacgtaaccct A/G cggctcaagatgccgccaca | 3720 | | |
| | | (Leu 1176 Leu)) | | | | |
| ABCA7 | 35 | (coding region 4046 | cccagcctgccagtgtagcc G/A gcccggtgcccggcgcctgc | 3721 | | |
| | | (Arg 1349 Gln)) | | | | |
| ABCA7 | 36 | (intron 30 81) | ccccctgggagctctcccgg C/A ccccccggccctcagctccc | 3722 | | |
| ABCA7 | 37 | (intron 32 1) | caaggagcagctgtctgagg G/C tgcactgtgagtccctccac | 3723 | | |
| ABCA7 | 38 | (intron 33 54) | ccactgcttgccactgccct G/A tctggccccttgtaggcagg | 3724 | | |
| ABCA7 | 39 | (intron 34 245) | cagtactttgggaggccgag G/A caggaggactgcttgtggcc | 3725 | | |
| ABCA7 | 40 | (coding region 5057 | ggtgagccggatcttgaaac A/G ggtcttccttatcttccccc | 3726 | | |
| | | (Gln 1686 Arg)) | | | | |
| ABCA7 | 41 | (intron 38 65) | ggcccactcacctttctgaa A/G gacctgcactctcccaggta | 3727 | | |
| ABCA7 | 42 | (intron 40 154) | ctctacctcccacacgcgga C/G caggccctgagacacccctg | 3728 | | |
| ABCA7 | 43 | (intron 40 277) | ctgagcccccggcgccccca T/C ccccagcgtggcccgggaac | 3729 | | |
| ABCA7 | 44 | (coding region 5592 | gtggcccgggaacccagtgc T/C gcgcacctcagcatgggata | 3730 | | |
| | | (Ala 1864 Ala)) | | | | |
| ABCA7 | 45 | (intron 41 286) | ctccttgactctgccttctg T/C ggccctgcccacttgctcct | 3731 | | |
| ABCA7 | 46 | (intron 41 389) | tggccgttcccagtttgcag C/T cgtttcactgcctcttccat | 3732 | | |
| ABCA7 | 47 | (intron 41 991) | cacactatggccctgcccca C/T ac c/t cat c/g cc a/g gctc | 3733 | | |
| ABCA7 | 48 | (intron 41 994) | actatggccctgcccca c/t ac C/T cat c/g cc a/g gctccac | 3734 | | |
| ABCA7 | 49 | (intron 41 998) | tggccctgcccca c/t ac c/t cat C/G cc a/g gctccacccac | 3735 | | |
| ABCA7 | 50 | (intron 41 1001) | ccctgcccca c/t ac c/t cat c/g cc A/G gctccacccacacc | 3736 | | |
| ABCA7 | 51 | (intron 41 1051) | actcatgctggctccaccca C/T accatggccccgccccatac | 3737 | | |
| ABCA7 | 52 | (intron 41 1131) | tgccctgccccatgcccatt A/G tgcccctgctccacactcaa | 3738 | | |
| ABCA7 | 53 | (coding region 5985 | gaagcgctctgctcgcgcct G/A gccatcatggtgaatgggcg | 3739 | | |
| | | (Leu 1995 Leu)) | | | | |
| ABCA7 | 54 | (intron 44 201) | ggcgcaggaccaggaggcgt G/C agccgggggctctgggtgga | 3740 | | |
| ABCA7 | 55 | (intron 44 233) | ctgggtggatttagaagaca C/T aatcaggtgtgcgttggagt | 3741 | | |
| ABCA7 | 56 | (intron 44 313) | agttaggggagggcctggtt A/G gtgggcggggccataggaaa | 3742 | | |
| ABCA7 | 57 | (coding region 6133 | tggcggccgagttccctggg G/T cggagctgcgcgaggcacat | 3743 | | |
| | | (Ala 2045 Ser)) | | | | |
| ABCA7 | 58 | (coding region 6159 | ctgcgcgaggcacatggagg C/T cgcctgcgcttccagctgcc | 3744 | | |
| | | (Gly 2054 Gly)) | | | | |
| ABCA7 | 59 | (intron 45 27) | acggcgccggggtcgggctg G/C gggaggcaggctgggggcca | 3745 | | |
| ABCA7 | 60 | (3′flanking region 108) | caagctgagtgtgcacatac G/A ggccaagtggcgattcatag | 3746 | | |
| ABCA7 | 61 | (3′flanking region 376) | cttacaggagcccggtgtcc C/T ggagcacaggccagggccgg | 3747 | | |
| ABCA7 | 62 | (3′flanking region 687) | cagcagggagacttggggag G/A g/a gggagagagttcacactgc | 3748 | | |
| ABCA7 | 63 | (3′flanking region 688) | agcagggagacttggggag g/a G/A gggagagagttcacactgcg | 3749 | | |
| ABCA7 | 64 | (3′flanking region 1169) | cctcgacctgacccacttca C/T ggggctgcagggcgggtgat | 3750 | | |
| ABCA7 | 65 | (intron 9 398-422) | cgtgaactaccacgtcctgc (T)22-26 aagagatggagtctcactct | 3751 | | |
| ABCA7 | 66 | (intron 12 175-184) | ggggactctgagggtctggt (G)8-10 actctgagggtctgggggcc | 3752 | | |
| ABCA7 | 67 | (intron 30 81-87) | ccccctgggagctctcccgg (C)6-7 ggccctcagctccccttccc | 3753 | | |
| ABCA7 | 68 | (intron 34 349-361) | agaaagagaaagagagaaag (A)12-14 cagaaatgtgctttgggtga | 3754 | | |
| ABCA8 | 1 | (intron 9 204) | ctggtaattaatattagata A/G ataaaaacattgagttagaa | 3755 | | |
| ABCA8 | 2 | (intron 12 266) | aacattatgttgttttaaac A/G taactgagtgtagaaataag | 3756 | | |
| ABCA8 | 3 | (intron 30 733) | ttgccatatgtataataaag T/A attcatgtttttgctagcct | 3757 | | |
| ABCA8 | 4 | (intron 34 861) | agactggagtttgcatgcta C/T ctaagactgtagctgattcc | 3758 | | |
| ABCA8 | 5 | (intron 1 907) | gaggagatcatcctcttggc C/T aatgtctattaacttcgcca | 3759 | | |
| ABCA8 | 6 | (intron 1 1262) | cagaaacttttgccctctct G/A taggctagctcactgtgaaa | 3760 | | |
| ABCA8 | 7 | (intron 1 1537) | agctctcttaaaagtatcca T/C gctgaattttctgcacctta | 3761 | | |
| ABCA8 | 8 | (intron 1 7622) | tcgttaacagcaatgataat T/C tagcccatccttatcc c/t a | 3762 | | |
| ABCA8 | 9 | (intron 1 7639) | t t/c tagcccatccttatcc C/T agaaacaacaggctcataag | 3763 | | |
| ABCA8 | 10 | (intron 1 7720) | tccatgtgttacaaactgcc C/T tggagaacagaaaaagagaa | 3764 | | |
| ABCA8 | 11 | (intron 1 9397) | cataatatatatacatatgc G/A cacacacacacatatacaca | 3765 | | |
| ABCA8 | 12 | (intron 1 9519) | ttgtttgcaggatatttctt T/C ttcttaagaacttcatatta | 3766 | | |
| ABCA8 | 13 | (intron 1 12973) | caattagttttcttcaaaaa A/G gtagaaaagttggaattgta | 3767 | | |
| ABCA8 | 14 | (intron 1 13100) | catataaaaaatcttgatta A/T actttggtatattttaaaaa | 3768 | | |
| ABCA8 | 15 | (intron 1 13128) | gcaatgccttggaactatct C/T ttaaaacacattgactttca | 3769 | | |
| ABCA8 | 16 | (intron 2 420) | caattagttttcttcaaaaa A/G gtagaaaagttggaattgta | 3770 | | |
| ABCA8 | 17 | (intron 2 505) | catataaaaaatcttgatta A/T actttggtatattttaaaaa | 3771 | | |
| ABCA8 | 18 | (intron 2 819) | gcaatgccttggaactatct C/T ttaaaacacattgactttca | 3772 | | |
| ABCA8 | 19 | (intron 3 915) | ttgtgttcgatagatcagta G/A ggtgactagttaacaatgat | 3773 | | |
| ABCA8 | 20 | (intron 3 1539) | aaagggaaatctgtggtgat C/T gccctgtcattcattcatag | 3774 | | |
| ABCA8 | 21 | (intron 3 2341) | ttcctttctttgtcaacttc C/T gtccaaattccactcaagct | 3775 | | |
| ABCA8 | 22 | (intron 3 2882) | tattctatattctgtactcc A/G ttaatattctataataataa | 3776 | | |
| ABCA8 | 23 | (intron 3 3314) | atttaaatatctatctctct A/G tatttaccatttcaaattta | 3777 | | |
| ABCA8 | 24 | (intron 4 89) | gaggttagtatgccaaatta G/A agcatcactatctgtcataa | 3778 | | |
| ABCA8 | 25 | (intron 4 3264) | ttccattggcctattatgcc C/T gtgttatatccagtgttaga | 3779 | | |
| ABCA8 | 26 | (intron 4 3403) | aagagaccaacaaaattctt C/G atcagcagaaaagcacagga | 3780 | | |
| ABCA8 | 27 | (intron 5 389) | gcttactgaatatataaatt G/C agaaaagccatgccaagcaa | 3781 | | |
| ABCA8 | 28 | (intron 5 479) | tgagagtggtgagtaactca A/G aatgcctggactcc g/a aggtc | 3782 | | |
| ABCA8 | 29 | (intron 5 494) | actca a/g aatgcctggactcc G/A aggtcccagcaggtcaatga | 3783 | | |
| ABCA8 | 30 | (coding region 792 | atgggtcttcgggattcagc G/A ttctggtgagtcaaacgcag | 3784 | | |
| | | (Ala 264 Ala)) | | | | |
| ABCA8 | 31 | (intron 6 200) | cctcccaagtagctgggact G/A caggtgccg a/g caccatgcc | 3785 | | |
| ABCA8 | 32 | (intron 6 210) | agctgggact g/a caggtgccg A/G ccaccatgcctggataattt | 3786 | | |
| ABCA8 | 33 | (intron 6 1751) | gtgagttattattgtgttgg C/T tttgcagctgttttgttttt | 3787 | | |
| ABCA8 | 34 | (intron 6 1808) | atttcattatagttttcaaa G/T aatattgtaaaacaaaagaa | 3788 | | |
| ABCA8 | 35 | (intron 6 2412) | tattcctaattctaaagaat T/C ctgcccaaaacttttacctt | 3789 | | |
| ABCA8 | 36 | (intron 6 2506) | tggatgaataagtgaatgaa G/A agttatcttaga a/g tccattt | 3790 | | |
| ABCA8 | 37 | (intron 6 2519) | gaatgaa g/a agttatcttaga A/G tccatttcaggtcttccttt | 3791 | | |
| ABCA8 | 38 | (intron 7 28) | agtgaattaaatatctttcc A/G tccacctatagcctaaaaat | 3792 | | (coding region 991 |
| ABCA8 | 39 | (Gly 331 Ser)) | taaagaaatctttcctcacc G/A gcctggtcgtgttcctcctc | 3793 | | |
| ABCA8 | 40 | (intron 8 74) | tggaatccataggctgtaat C/T atttacaaactcagcattgt | 3794 | | |
| ABCA8 | 41 | (intron 9 1417) | acacatacttaaatatattt T/C ctctgttctacttttgtttt | 3795 | | |
| ABCA8 | 42 | (intron 9 2504) | agaggaaaattatggtttgg G/A aatgaaataaagcagaaata | 3796 | | |
| ABCA8 | 43 | (intron 10 2013) | tggccaaagatctttccaac C/T tgtgccagtggttcacagga | 3797 | | |
| ABCA8 | 44 | (intron 10 2378) | ctgaagaaaattgtcacttt G/A aagtatcttttctttttttc | 3798 | | |
| ABCA8 | 45 | (intron 11 − 697) | aaaaaaaaaaaaaaagagag A/G gagaaagaaaatatttgtta | 3799 | | |
| ABCA8 | 46 | (intron 11 − 528) | tataaaagttagaaaaaaat G/T a a/g tatgttttagaaatagat | 3800 | | |
| ABCA8 | 47 | (intron 11 − 526) | taaaagttagaaaaaaaaat g/t a A/G tatgttttagaaatagat | 3801 | | |
| ABCA8 | 48 | (intron 11 − 342) | ctcaaaggagttttagccat G/A taataacttactattaatct | 3802 | | |
| ABCA8 | 49 | (coding region 1632 | ggttcagtcaccatctataa C/T aataagctttcagaaatggc | 3803 | | |
| | | (Asn 544 Asn)) | | | | |
| ABCA8 | 50 | (intron 14 252) | cttattgcaaaataagtgaa G/A ttgagtttctaagagatcaa | 3804 | | |
| ABCA8 | 51 | (intron 15 130) | ttttgtttttgagacggagt A/C tcgatcatctcggctcactg | 3805 | | |
| ABCA8 | 52 | (intron 16 534) | acatatacattcattcaaat A/G cacattttatggtgacaaca | 3806 | | |
| ABCA8 | 53 | (intron 16 588) | gaatcatcaggaaagtgtta C/T gcaaattctgattagtactt | 3807 | | |
| ABCA8 | 54 | (intron 16 645) | atttaaagaaaatttgtaga C/T gttttaggtggaatgaagaa | 3808 | | |
| ABCA8 | 55 | (intron 17 431) | tgtcaggtttttcttttttt T/A ttctttatgttagaaattgg | 3809 | | |
| ABCA8 | 56 | (intron 17 1390) | gctgtaaactcgttttgtga C/A ttaggtaccccatgattcta | 3810 | | |
| ABCA8 | 57 | (intron 17 2452) | cacgttatacctatagtaac G/A cggaaga g/c tctaatcatgag | 3811 | | |
| ABCA8 | 58 | (intron 17 2460) | acctatagtaac g/a cggaaga G/C tctaatcatgagat g/c ctt | 3812 | | |
| ABCA8 | 59 | (intron 17 2475) | gaaga g/c tctaatcatgagat G/C cttagcagagccaatctcta | 3813 | | |
| ABCA8 | 60 | (intron 18 152) | gaagaagcacaggagagagg C/T agaatcttgacatccaaagg | 3814 | | |
| ABCA8 | 61 | (intron 19 7477) | aaaatctattttgaaagaca C/T ttggaactaaaaaaatcttt | 3815 | | |
| ABCA8 | 62 | (intron 21 196) | ttgtttaaagtaaaataaaa T/C g/c aacaaaacatttttcaaag | 3816 | | |
| ABCA8 | 63 | (intron 21 197) | tgttaaagtaaaataaaa t/c G/C aacaaaaacatttttcaaaga | 3817 | | |
| ABCA8 | 64 | (intron 21 287) | actgtggtggggtgggggga G/T ggggaggggatagcattggg | 3818 | | |
| ABCA8 | 65 | (intron 21 403) | cctgcacaatgtgcacatgt A/G ccctaaaacctaaagtataa | 3819 | | |
| ABCA8 | 66 | (intron 21 1207) | cccagcc g/a gagtgcagtggc A/G ggatcatagctcactgtaac | 3820 | | |
| ABCA8 | 67 | (intron 24 692) | ctcctagatatagacaaaaa A/C caaggtgcacaatggccatg | 3821 | | |
| ABCA8 | 68 | (intron 25 212) | cctgattaatatatgggaag G/A aagggtaaggggtagtggga | 3822 | | |
| ABCA8 | 69 | (intron 26 67) | aataattttcagttctgtac A/G cactgtgaaacttcttttat | 3823 | | |
| ABCA8 | 70 | (intron 27 515) | gtgtctcccaaaccacatca G/T tttcatcttttgctattaca | 3824 | | |
| ABCA8 | 71 | (intron 27 661) | cctggatattatcagactta G/A aatggagaggaaaagtcaat | 3825 | | |
| ABCA8 | 72 | (intron 30 1967) | caaaaattagatacaagggg G/C tgaaattgactttaattgta | 3826 | | |
| ABCA8 | 73 | (intron 31 112) | ctctaaatgctgacccaggt C/G acactgggtagatttacaac | 3827 | | |
| ABCA8 | 74 | (intron 33 401) | cttctcactaggttgtgaga C/T gctgttgttaaattttatgt | 3828 | | |
| ABCA8 | 75 | (intron 35 484) | taacagcatcatcctg a/t tgt A/G tttattttcatagacagaaa | 3829 | | |
| ABCA8 | 76 | (intron 36 258) | tttgcatgtatgttggtaaa A/G cctaagtcaaaactcagtta | 3830 | | |
| ABCA8 | 77 | (intron 36 375) | atattattttactgtcttag C/G ctgtatattaagaaactgac | 3831 | | |
| ABCA8 | 78 | (3′flanking region 674) | gcggtggacatagaaagccc G/A gaagcttcttgatgtgctta | 3832 | | |
| ABCA8 | 79 | (intron 1 56-57) | ttttgcttttgtgtgtgagt TT/Δ gtttcagaggttttgtcttt | 3833 | | |
| ABCA8 | 80 | (intron 1 1180-1191) | taaagtataataataaaacg (A)9-11 gaaattcctcctgtacagag | 3834 | | |
| ABCA8 | 81 | (intron 1 9877-9885) | ctcctgcaaataggtatgac (A)8-12 tcaactgagtacaaaaagct | 3835 | | |
| ABCA8 | 82 | (intron 1 12588) | gtactagagtgcactccttt T/Δ gcaacaggacggccaaagga | 3836 | | |
| ABCA8 | 83 | (intron 6 78) | tcaatgcatctttttttttt T/Δ gaaatggagtctcgctctgt | 3837 | | |
| ABCA8 | 84 | (intron 9 265) | gtatatggtatttttttttt T/Δ gcaacaggacggccaaaaga | 3838 | | |
| ABCA8 | 85 | (intron 9 2666) | attttttttaaaggtatcca A/Δ tagtcattctcaatttcttc | 3839 | | |
| ABCA8 | 86 | (intron 11 − 447) | ggatattctgggtttttttt T/Δ ctacaaactcaagttttttg | 3840 | | |
| ABCA8 | 87 | (intron 15 8407) | gtggaataatttttgactta T/Δ gcatttggtcaaataaaatt | 3841 | | |
| ABCA8 | 88 | (intron 15 9458-9470) | tatgtcgagtaacatatgtc (T)11-15 ctgaatgccagcttgcagtt | 3842 | | |
| ABCA8 | 89 | (intron 16 54-56) | tgaataatagtcatcatcat CAT/Δ aattattatcattacaacta | 3843 | | |
| ABCA8 | 90 | (intron 17 433) | tcaggtttttcttttttttt t/a T/Δ ctttatgttagaaattggac | 3844 | | |
| ABCA8 | 91 | (intron 24 1462) | actccatctcaaaaaaaaaa A/Δ gagagaaaaaaattctgcat | 3845 | | |
| ABCA8 | 92 | (intron 33 155) | caatactttgcaaaaaaaaa A/Δ gatctttccctgatgatatt | 3846 | | |
| ABCA8 | 93 | (intron 34 184) | atactgaatggttttttttt T/Δ ctcctttctcatatgacctc | 3847 | | |
| ABCA8 | 94 | (3′flanking region 1240) | atccttggaccaaaaaaaaa A/Δ ctttatctgtgctttgcgtg | 3848 | | |
| ABCB1 | 1 | 5′flanking − 196 | gctttggagccatagtcatg T/C actcaaaatttattttatct | 3849 | | |
| ABCB1 | 2 | 5′flanking − 16 | tactctttacctgtgaagag T/C agaacatgaagaaatctact | 3850 | | |
| ABCB1 | 3 | intron 1 + 71660 | cttgctggaggaagggtgct A/C gaaaatataccaaatccaag | 3851 | | |
| ABCB1 | 4 | intron 1 + 80091 | gaaataatattcaagttctg A/C aataatatcatgacctatag | 3852 | | |
| ABCB1 | 5 | intron 1 + 103126 | gatatgaatcagaattcatc T/C gtgtctcaagaaaaggtcat | 3853 | | |
| ABCB1 | 6 | intron 1 + 103148 | tgtctcaagaaaaggtcatg C/T gataaattaagttctgctag | 3854 | | |
| ABCB1 | 7 | intron 1 + 108428 | aattaatttatcatcatctg A/G tcaccatttcacacaactca | 3855 | | |
| ABCB1 | 8 | intron 1 + 112042 | cataagttgaaatgtcccca A/G tgattcagctgatgcgcgtt | 3856 | | |
| ABCB1 | 9 | intron 2 + 491 | gctctctggcttcgacgggg G/Δ actagaggttagtctcacct | 3857 | | |
| ABCB1 | 10 | intron 4 + 36 | attaactattcaaaatactt C/T ggaaatttgacatctcctta | 3858 | | |
| ABCB1 | 11 | intron 5 + 1596 | ttagctctcttactgcttca T/C agtggaagaatcaaatactt | 3859 | | |
| ABCB1 | 12 | intron 8 + 1789 | aaacactctgaatattaaac C/T gctcctggaaccacagctca | 3860 | | |
| ABCB1 | 13 | intron 14 + 24 | agttgtccttgccctttgcc T/C ttctagaggtgcaaaaaata | 3861 | | |
| ABCB1 | 14 | intron 14 + 81 | tgcaggaagttaggaaacta C/T tataaatcggaagaagggaa | 3862 | | |
| ABCB1 | 15 | intron 15 + 38 | caaaccaacctgatttataa A/G aaaggaatgctctctgttta | 3863 | | |
| ABCB1 | 16 | intron 17 + 73 | gtttggtgggctagggctac A/G gtaggagtgggaacaagaga | 3864 | | |
| ABCB1 | 17 | intron 18 + 564 | caacagtaaagttacaatct G/A aaaggaatgctctctgttta | 3865 | | |
| ABCB1 | 18 | intron 18 + 2062 | tttccctgaggaatggttat C/T ctctgtgttccttgagtcca | 3866 | | |
| ABCB1 | 19 | intron 18 + 2293 | ccacatcaggttttccccag A/G ggttttagttcctcccctca | 3867 | | |
| ABCB1 | 20 | intron 20 + 557 | aaaaccctaaccattgacac G/A tgtgaatgttttcctgggga | 3868 | | |
| ABCB1 | 21 | intron 21 + 24 | cgtgcctcctttctactggt G/A tttgtcttaattggccattt | 3869 | | |
| ABCB1 | 22 | intron 21 + 2725 | ctgacctgtttttggctgac A/G ggttttagttcctcccctca | 3870 | | |
| ABCB1 | 23 | intron 21 + 4725 | tcttggtattaaaagatcca A/G agagataggaatatgtaatt | 3871 | | |
| ABCB1 | 24 | intron 22 + 8507 | tgcacttaggaaaaaaacaa T/C atggaaatgtgtaaaatata | 3872 | | |
| ABCB1 | 25 | intron 22 + 8537 | tgtaaaatatactttttttt T/A aaaaaaaaggacacatttat | 3873 | | |
| ABCB1 | 26 | intron 22 + 8565 | aggacacatttattcagcat T/C atgatcagactattacattt | 3874 | | |
| ABCB1 | 27 | intron 22 + 8952 | caccttggtttcatggtttg G/A caaagtactggcctgtacca | 3875 | | |
| ABCB1 | 28 | intron 22 + 9520 | caccaacaaatatctttttc A/G cagttgggtgggcatctggt | 3876 | | |
| ABCB1 | 29 | intron 22 + 9836 | agactctgacttagacatga C/T ggcaggggaaagagagactt | 3877 | | |
| ABCB1 | 30 | intron 24 + 377 | taaaatacagatgtgttgta C/A taagttctgcaagcctttgg | 3878 | | |
| ABCB1 | 31 | intron 24 + 1493 | ggggaggtgtccaggcacga A/Δ catggagagctggacttgat | 3879 | | |
| ABCB1 | 32 | intron 24 + 1495 | ggaggtgtccaggcacgaac A/T tggagagctggacttgatac | 3880 | | |
| ABCB1 | 33 | intron 25 + 342 | tgcagccttgatcttctggg C/T tcaagcgatcctcctgcctc | 3881 | | |
| ABCB1 | 34 | intron 26 + 134 | cttggataaagtctgagagc C/G taaatatggtctccaagtgg | 3882 | | |
| ABCB1 | 35 | intron 26 + 1272 | gtccttcaattttgtggtga A/G cttaaaaacaggactctaaa | 3883 | | |
| ABCB1 | 36 | intron 26 + 1394 | tattaagtggtgtgttaaag A/G ttgtgctataatgaattgta | 3884 | | |
| ABCB1 | 37 | intron 26 + (1987-1988) | aagggctggaagagtgaaag (AAAG) gaggctatttgctcccagac | 3885 | | |
| ABCB1 | 37 | intron 26 + (1987-1988) | aagggctggaagagtgaaag gaggctatttgctcccagac | 3886 | | |
| ABCB1 | 38 | intron 27 + 59 | gcagcctctctggcctatag G/T ttgatttataaggggctggt | 3887 | | |
| ABCB1 | 39 | intron 27 + 80 | ttgatttataaggggctggt C/T caatgactgagtcaagaatt | 3888 | | |
| ABCB4 | 1 | exon 3 + 3 | aacacccttattttatagat C/T caatgactgagtcaagaatt | 3889 | | |
| ABCB4 | 2 | intron 3 + 45 | cagcatctctacttatacca T/C gctctgctttaaggttctct | 3890 | | |
| ABCB4 | 3 | intron 3 + 498 | actcaaataggtggtaggag C/T agagacaattcaatacagac | 3891 | | |
| ABCB4 | 4 | intron 3 + 515 | gagcagagacaattcaatac A/G gacagaagtcttagatgaga | 3892 | | |
| ABCB4 | 5 | intron 6 + 1030 | tagttttgccatgtagaatt G/C aaaaagtgatagatggtgtt | 3893 | | |
| ABCB4 | 6 | intron 6 + 1437 | gttaagcctgcttcaatcaa G/A ttagttatattcttgttcta | 3894 | | |
| ABCB4 | 7 | intron 6 + 2449 | ttgacttagcgacactgtta G/A catacttatctttcctgtgt | 3895 | | |
| ABCB4 | 8 | intron 7 + 451 | ccttgctgcacctgtgctgt A/C taagtttggcttattatagt | 3896 | | |
| ABCB4 | 9 | intron 7 + 530 | agtagagacaggctggcgat C/G acaccggacagagctaactg | 3897 | | |
| ABCB4 | 10 | intron 7 + 152 | aacagaatcatgaaattaag T/C tgttaatgatttgaaggcct | 3898 | | |
| ABCB4 | 11 | exon 8 + 40 | aggataaattgtttatgtcg C/T tgttaatgatttgaaggcct | 3899 | | |
| ABCB4 | 12 | intron 8 + 130 | ctggttgactccagatatca T/C agaaggagttgtaaaattct | 3900 | | |
| ABCB4 | 13 | intron 8 + 248 | aatacacaggaagcttctaa A/G taaagtaaggaagtcactct | 3901 | | |
| ABCB4 | 14 | intron 8 + 531 | ctaaagagtgaatggattca A/G tacgtcccttggaactcacc | 3902 | | |
| ABCB4 | 15 | intron 8 + 4240 | ctgaggttccagcttatctc T/A tagagatgtttacttagtct | 3903 | | |
| ABCB4 | 16 | intron 8 + 4343 | tgttagaagaaaaaaaggtt C/T atattacaagagggtctgac | 3904 | | |
| ABCB4 | 17 | intron 8 + 4677 | cccaagatatcttcataact G/C tccatagtgcctagggtgcc | 3905 | | |
| ABCB4 | 18 | intron 9 + 113 | tttacccagattcacctatt A/G ttatcatttttgctcccaaa | 3906 | | |
| ABCB4 | 19 | intron 9 + 982 | tgtcctatacagtttttgtt T/A taagtttagtaaattgatta | 3907 | | |
| ABCB4 | 20 | intron 11 + 241 | gcactttgggaggccaaggt A/G cataaatcacttgaggtcag | 3908 | | |
| ABCB4 | 21 | intron 11 + 457 | tccagcttgggtgacagagt A/G agacttcatctcaaaaaaaa | 3909 | | |
| ABCB4 | 22 | intron 11 + 1337 | tactcttggggagcctatca C/G cagggtgggtcagatatagc | 3910 | | |
| ABCB4 | 23 | exon 12 + 3 | tgtttcttttctgtccagat A/T ctctcggcatttagtgacaa | 3911 | | |
| ABCB4 | 24 | intron 12 + 1288 | cagaccacactaaccctcag T/C tggacctcaggatgtcagtg | 3912 | | |
| ABCB4 | 25 | intron 13 + 206 | tgtggataagaaaatagcat G/A tggttagaccatttgtgaaa | 3913 | | |
| ABCB4 | 26 | intron 13 + 988 | cagtcggtttggaagcttgc T/C accctttcttcacttcctca | 3914 | | |
| ABCB4 | 27 | intron 13 + (1413-1414) | tttatcttcacttatgtttt (T) ctcagttaagttatgctaat | 3915 | | |
| ABCB4 | 27 | intron 13 + (1413-1414) | tttatcttcacttatgtttt ctcagttaagttatgctaat | 3916 | | |
| ABCB4 | 28 | intron 13 + 1931 | cttgcaaatgttgctcttcc A/G caaaaaaaaaaggaaaggat | 3917 | | |
| ABCB4 | 29 | intron 22 + 767 | acagtgggctgatgcataga A/Δ cctgtagcaatccaccagca | 3918 | | |
| ABCB4 | 30 | intron 23 + 784 | agtatctcctaaactcttgc T/C atgcaggaaaaattatttta | 3919 | | |
| ABCB4 | 31 | intron 25 + 158 | gaaatattttactgtattaa T/C gtctagaacttaaatataag | 3920 | | |
| ABCB4 | 32 | intron 25 + 2920 | ctgagtcttcctatacatct T/A ttccattcctcggatgctgt | 3921 | | |
| ABCB4 | 33 | intron 29 + 411 | cttctcttaccttgaattct A/C ggctctcgaactttgacttt | 3922 | | |
| ABCB4 | 34 | 3′flanking + 458 | agaaaatgaaattgccctac T/C gagctaactctgaaagcaca | 3923 | | |
| ABCB7 | 1 | intron 1 + 220 | acggggcaggaggttctggg C/A agaggacacctggagcgctg | 3924 | | |
| ABCB7 | 2 | intron 1 + 480 | agttaactcccttgctgaca G/A gcgtgcttcttgataggcca | 3925 | | |
| ABCB7 | 3 | intron 1 + (512-513) | gataggccaaaaccgtaact AT/Δ ctttccaaaacatagaccgc | 3926 | | |
| ABCB7 | 4 | intron 1 + 1690 | agttctccaataaggcagat G/A aagttaagataaaatttgta | 3927 | | |
| ABCB7 | 5 | intron 1 + 5309 | aattaatatcatttattgct G/A tattgttgtcagtgttatct | 3928 | | |
| ABCB7 | 6 | intron 1 − 11274 | tgcttcttttcaagccagcc A/G gctttaaaaaaaagttagct | 3929 | | |
| ABCB7 | 7 | intron 1 − 11085 | caggttttcagggctcatgt A/G gacctgaagaaaaatgagag | 3930 | | |
| ABCB7 | 8 | intron 1 − 10037 | attctactttctcaacttct T/C ttattacattatctcatcat | 3931 | | |
| ABCB7 | 9 | intron 1 − 21 | ccactctgaaacttccccct G/A ctttttttccttgtcagcag | 3932 | | |
| ABCB7 | 10 | intron 3 + (135-136) | ttctctaatgaaaaaaaaaa (A) catattaattgaccatagtt | 3933 | | |
| ABCB7 | 10 | intron 3 + (135-136) | ttctctaatgaaaaaaaaaa catattaattgaccatagtt | 3934 | | |
| ABCB7 | 11 | intron 3 + 333 | aaaacaatttgtgtgtgtgc G/A tgtgcttcaaggttaatgtt | 3935 | | |
| ABCB7 | 12 | intron 12 + 524 | taaccactctgccctcagta C/T gaaacacagtgccgaaccca | 3936 | | |
| ABCB7 | 13 | intron 13 + 1543 | atcctgtgaggtggggaagc G/A tatggctagcataaatataa | 3937 | | |
| ABCB7 | 14 | intron 13 + 2400 | tgttaccttactgcctcatt C/G tcattcttcccacctgctat | 3938 | | |
| ABCB7 | 15 | intron 15 + 2201 | ctccttcctaaccttagcaa G/C agtctggagatttacttatc | 3939 | | |
| ABCB8 | 1 | 5′flanking − 2272 | ggcttaggcctaagggctga T/C gttggggccagtacccctga | 3940 | | |
| ABCB8 | 2 | 5′flanking − 2070 | agctatgaaaacaagaccct G/A tccttctagaggtagcaaaa | 3941 | | |
| ABCB8 | 3 | intron 1 + 25 | aaacggaaaaacctactcag C/G ccctctgtctccccattcca | 3942 | | |
| ABCB8 | 4 | exon 2 + 308 | tgctggtcctgggggtagcc G/A tcgtggtgaggctttcccca | 3943 | | |
| ABCB8 | 5 | intron 2 + 334 | cccccacttaaaacatttgt C/G ccctctgtctccccattcca | 3944 | | |
| ABCB8 | 6 | intron 4 + 12 | cctgctccggtactgccagc C/T gcagggtgcagagttggggt | 3945 | | |
| ABCB8 | 7 | intron 5 + 547 | agttcatagcattctcgctc G/A gccccctcaggcctgctgct | 3946 | | |
| ABCB8 | 8 | exon 7 + 57 | ggcaatgtgcggactgtgcg A/T gccttcgccatggagcaacg | 3947 | | |
| ABCB8 | 9 | intron 9 + 1231 | tttccgcagctgcatggaca C/T cctcgcgtgccccgtttctg | 3948 | | |
| ABCB8 | 10 | intron 9 + 2164 | cctcttggaggtccttctag C/T gctgcctatgtggagattct | 3949 | | |
| ABCB8 | 11 | intron 9 + 2645 | ttcctgcctggtgcctcccc C/Δ ggctgcctttagcaagtgct | 3950 | | |
| ABCB8 | 12 | intron 9 + 2646 | tcctgcctggtgcctccccc G/A gctgcctttagcaagtgctg | 3951 | | |
| ABCB8 | 13 | intron 9 + 3229 | cagggccgagcagggagtcc G/A tgggtcagctgggctccctt | 3952 | | |
| ABCB8 | 14 | intron 12 + (113-114) | tcctccactgccacaagggg (GG) ccttctttcctgggacaatc | 3953 | | |
| ABCB8 | 14 | intron 12 + (113-114) | tcctccactgccacaagggg ccttctttcctgggacaatc | 3954 | | |
| ABCB8 | 15 | intron 13 + 128 | tgctctcgggagaccctggc C/T gtcttcacatgtcctcagct | 3955 | | |
| ABCB8 | 16 | intron 13 + 305 | atccaggtctagagaagccc A/G tagtggaggtgctgagctgc | 3956 | | |
| ABCB8 | 17 | intron 14 + 135 | acagttgtgtcagggaagac C/G agaaccacagccaaagggga | 3957 | | |
| ABCB8 | 18 | intron 14 + 159 | accacagccaaaggggacag A/T gtcgttgtgtggggacaggg | 3958 | | |
| ABCB8 | 19 | intron 15 + 747 | gttggagccttgggctctgt A/G agggggacagagggaatcat | 3959 | | |
| ABCB8 | 20 | 3′flanking + 333 | cctatcccctggctcacccc G/A ggacccacagtccccatctt | 3960 | | |
| ABCB8 | 21 | 3′flanking + 1168 | ccctctttcaggggtgtgat G/A cagtgcattgatggagcagc | 3961 | | |
| ABCB8 | 22 | 3′flanking + (1719-1721) | tagaccgcaggagccgcgcc GTC/Δ ttcctaacctcgcctcggcc | 3962 | | |
| ABCB9 | 1 | intron 1 + 69 | agggtgccaggccaggcacg G/C gttagggggcgtctgggcac | 3963 | | |
| ABCB9 | 2 | intron 1 + 8873 | tgggcccagcagctggggcc T/C ggaactacctcaaaggcttc | 3964 | | |
| ABCB9 | 3 | intron 1 + 8940 | accagctcagcctgcccagc G/A tgcacacggcaccaagctgg | 3965 | | |
| ABCB9 | 4 | intron 1 + 11410 | agatccaagggatccagagg T/C tggaatgtgaccctccgtgc | 3966 | | |
| ABCB9 | 5 | intron 1 + 12863 | gggaagccagatgcccacaa G/A gctctgtgacttcacttcca | 3967 | | |
| ABCB9 | 6 | intron 1 + 19731 | gccaagtgtcaagatcgagc G/A aggggagggcctgacgaggg | 3968 | | |
| ABCB9 | 7 | intron 1 + 29649 | cagaatccagatgcccgtaa T/C gttgttaagaagcctgcaca | 3969 | | |
| ABCB9 | 8 | intron 1 + 31793 | ggccaggcggggaggggtac C/T ggccagaccggtgggcaaaa | 3970 | | |
| ABCB9 | 9 | intron 1 + 37537 | agagtcacagggttggggtg C/A ccccgggaaggtggcatcta | 3971 | | |
| ABCB9 | 10 | intron 1 + 38293 | taccagccctgtgctttcag G/A gaccatgtgacctgtcaact | 3972 | | |
| ABCB9 | 11 | intron 1 + 44661 | cccgaggtgcctggcttcac A/G gcaggattgccgtcctgcag | 3973 | | |
| ABCB9 | 12 | intron 1 + 49576 | aaagtggccccgtggcttgt C/T ccctgaagccctaaagcacc | 3974 | | |
| ABCB9 | 13 | intron 1 + 64669 | ccacagacaagccgggtagc C/A cacctcgcagctcaacacac | 3975 | | |
| ABCB9 | 14 | exon 2 + 448 | cctggttttgggccctgttc G/A tgtggacgtacatttcactc | 3976 | | |
| ABCB9 | 15 | intron 7 + 3364 | ggtaccaggagtcgggtatc A/G gtgggacaggaacgcgtgtc | 3977 | | |
| ABCB9 | 16 | intron 11 + 113 | gggccccaggagctctccca G/T actatcagcctcctgggctg | 3978 | | |
| ABCB9 | 17 | exon 12 + 370 | cccaggcctgcagcactgaa A/G gacgacctgccatgtcccat | 3979 | | |
| ABCB10 | 1 | 5′flanking − 424 | tcgcgtctgcgcgctccgcc C/T ggtctgccggcgtgagaaag | 3980 | | |
| ABCB10 | 2 | intron 1 + 491 | acaaggggcggttgcgcccc G/T cagcggccggactcccggag | 3981 | | |
| ABCB10 | 3 | intron 1 + 37 | ccacttccctccgccgggcc T/G ctccttctccacacgcgggg | 3982 | | |
| ABCB10 | 4 | intron 1 + 217 | actcgtttgcagattttaca C/T ttgttttcttgttgacacac | 3983 | | |
| ABCB10 | 5 | intron 1 + 405 | gcgtttatactttttttttt T/Δ aaccaaaaacacattatttg | 3984 | | |
| ABCB10 | 6 | exon 3 + 185 | agggccggggcccaggcttc C/T gtaggcatcagtatgatggt | 3985 | | |
| ABCB10 | 7 | intron 6 + 1269 | caaattcacaactgtgcctt C/G cacagaatgggttggaaaac | 3986 | | |
| ABCB10 | 8 | intron 9 + 632 | ccccactccacttgggtgag G/A gcaggtggatggtgatgggt | 3987 | | |
| ABCB10 | 9 | intron 10 + 2373 | tacctcagggcactcagaca G/C cctcaccaatcagaggctca | 3988 | | |
| ABCB10 | 10 | intron 11 + 108 | tccttttcctgttttttgtt T/G ttttttttttcttggagtgg | 3989 | | |
| ABCB10 | 11 | intron 11 + 2379 | cattggtttttagtgtattc T/A gtgttgtgcatccatcatca | 3990 | | |
| ABCB11 | 1 | 5′flanking − (2596-2595) | tgtggtttagagctttctct (TT) gagacatttttgctaaggtt | 3991 | | |
| ABCB11 | 1 | 5′flanking − (2596-2595) | tgtggtttagagctttctct gagacatttttgctaaggtt | 3992 | | |
| ABCB11 | 2 | 5′flanking − 1746 | agctgaagtgaattaagcac G/A atcaactcagtactcacact | 3993 | | |
| ABCB11 | 3 | 5′flanking − (326-314) | agggggaaagtttaaaggta (T)9-12 gtcttgttatgtttttaagt | 3994 | | |
| ABCB11 | 4 | 5′flanking − 135 | agagggtttcccaagcacac T/C ctgtgtttggggttattgct | 3995 | | |
| ABCB11 | 5 | intron 1 + 511 | aaatatagatgcaaaaaaaa A/Δ tgagctgtggatgcatgttt | 3996 | | |
| ABCB11 | 6 | intron 1 + 581 | aatttcagtttttaggtcac C/T caagccagtgggagtcacat | 3997 | | |
| ABCB11 | 7 | intron 1 + (1938-1951) | aaagacgttttaagggcttt (A)10-13 gaaagaaaagaaaactgtag | 3998 | | |
| ABCB11 | 8 | intron 1 + 4517 | aagagatggtctctagcccc CT/Δ gtttgatttggggcacttac | 3999 | | |
| ABCB11 | 9 | intron 1 + 5651 | aaagagaataggttagtgga T/C tagtattcctgtgcttaatg | 4000 | | |
| ABCB11 | 10 | intron 1 + (12200-12201) | aagagatggtctctagcccc CT/Δ gtttgatttggggcacttac | 4001 | | |
| ABCB11 | 11 | intron 1 + 13023 | gtttggctactttgattaaa G/A aagaaagaagagataataat | 4002 | | |
| ABCB11 | 12 | intron 2 + 739 | cctgcatctattctgaccta C/T actggggaaaacagtatgtg | 4003 | | |
| ABCB11 | 13 | intron 2 + (921-922) | tattttgtagttcaaaaagt | 4004 | | |
| | | | (CAGATCTTCTTCAGCTAATTTAGAAATGT) | | | |
| | | | tgctgtccatttgatattca | | | |
| ABCB11 | 13 | intron 2 + (921-922) | tattttgtagttcaaaaagt | 4005 | | |
| | | | tgctgtccatttgatattca | | | |
| ABCB11 | 14 | intron 3 + 644 | agccacacgtttcttattgc G/A tgggaagtttaaaaaatggg | 4006 | | |
| ABCB11 | 15 | intron 3 + 2231 | agtgaacctgagattgagct A/G tactgaaatctctagaagag | 4007 | | |
| ABCB11 | 16 | intron 3 + 2406 | aaagggtggtctttaaatcc T/C tatgtttttctcatcaggtt | 4008 | | |
| ABCB11 | 17 | exon 4 + 10 | tttctcatcaggttacaaga T/C gagaagaaaggtgatggcgt | 4009 | | |
| ABCB11 | 18 | intron 4 + 434 | acaatttatagtatttctca A/G tgccccacacagtttatcta | 4010 | | |
| ABCB11 | 19 | intron 4 + 518 | gtagatgagtagctaaaaac G/T aaagtcagctcctgaaataa | 4011 | | |
| ABCB11 | 20 | exon 5 + 120 | ggcacaatgacagatgtttt T/C attgactacgacgttgagtt | 4012 | | |
| ABCB11 | 21 | intron 5 + 320 | gggaggtgacccatgaattt T/C acttgagtatcatctccaag | 4013 | | |
| ABCB11 | 22 | intron 5 + 16076 | agaagaggtaacagtaagcc T/G cctgatttacagcacacatc | 4014 | | |
| ABCB11 | 23 | intron 6 + 303 | atttgcaggtgtgtttgtag G/C gggcagttgagtagcttgaa | 4015 | | |
| ABCB11 | 24 | intron 7 + 1141 | aaaggattcagcaggcatga A/G gaaagaaaagctttgcaaga | 4016 | | |
| ABCB11 | 25 | intron 8 + 2463 | ccattggctaatagcaatga A/C ctatgacatggtctaactta | 4017 | | |
| ABCB11 | 26 | intron 8 + 2677 | tcaatgatgttacagtgaga A/C tctaatattgtattaaaccc | 4018 | | |
| ABCB11 | 27 | intron 8 + 2699 | ctaatattgtattaaaccca T/A gccacatgttaaatgaatct | 4019 | | |
| ABCB11 | 28 | exon 9 + 24 | gtgtccaagtttacggacta T/C gagctgaaggcctatgccaa | 4020 | | |
| ABCB11 | 29 | intron 9 + 108 | caccttggtctgtggcctcc A/G gaggaagtacttgttcaaga | 4021 | | |
| ABCB11 | 30 | intron 10 + 2475 | taatcattccaaaccacgga C/A tttatttcattaagaacatg | 4022 | | |
| ABCB11 | 31 | intron 10 + 2478 | tcattccaaaccacggactt T/A atttcattaagaacatgata | 4023 | | |
| ABCB11 | 32 | intron 10 + 2711 | tttacagattggaaaagcca C/T tgaagtattgcaggtccaga | 4024 | | |
| ABCB11 | 33 | intron 10 + 3539 | agtgactgtaattagtatca C/G ttgtgcacagagaaaaaatg | 4025 | | |
| ABCB11 | 34 | intron 10 + 3623 | tgcagaaggttgttctttca T/C gaccttcctgagtttcagaa | 4026 | | |
| ABCB11 | 35 | intron 10 + 3661 | gaattcattaataaaaataa A/T cacataatggagcgtgacat | 4027 | | |
| ABCB11 | 36 | intron 10 + 5100 | gggccactctttggcttggc A/G atagactgtggccaatgaaa | 4028 | | |
| ABCB11 | 37 | intron 10 + 5292 | gctatttggtaggaacatct G/A ggcatgatcaggtagccttc | 4029 | | |
| ABCB11 | 38 | intron 10 + 5912 | gagtaatattcagtaaaaaa A/Δ taaagtggtattttaaatca | 4030 | | |
| ABCB11 | 39 | intron 12 + 116 | tgtttccagtaatagggaat G/A gaggtgtctttctctgaaag | 4031 | | |
| ABCB11 | 40 | intron 12 + 326 | gataaatgacaaggcaatta G/C aacaatcaggaagcacaggt | 4032 | | |
| ABCB11 | 41 | intron 12 + 335 | caaggcaattacaacaatca A/G gaagcacaggttcttcccaa | 4033 | | |
| ABCB11 | 42 | intron 12 + 2572 | cctcatccttgccaatgttt C/T cttttactggtttttgatgg | 4034 | | |
| ABCB11 | 43 | exon 13 + 23 | tctaaatgacctcaacatgg T/C cattaaaccaggggaaatga | 4035 | | |
| ABCB11 | 44 | intron 13 + 70 | atggcagtatattgatcaaa C/T agaaaggtgtagcatacatt | 4036 | | |
| ABCB11 | 45 | intron 13 + (1578-1579) | ttattggcctctattttttc (C) tgcccattggtcaagtatga | 4037 | | |
| ABCB11 | 45 | intron 14 + (1578-1579) | ttattggcctctattttttc tgcccattggtcaagtatga | 4038 | | |
| ABCB11 | 46 | intron 14 + 32 | catacattcctgggagaaac C/T aagaggtcatagaaggaaaa | 4039 | | |
| ABCB11 | 47 | intron 14 + 80 | cacaattatacacatttctt C/T tcgtatgattcccaagtcat | 4040 | | |
| ABCB11 | 48 | intron 14 + 439 | tattgtgtcaaaaacaattc A/G ttgtatatctccattctaag | 4041 | | |
| ABCB11 | 49 | intron 14 + (1262-1263) | cagcctttgcattatatttt (T) gctgtgttgtctaacaggag | 4042 | | |
| ABCB11 | 49 | intron 14 + (1262-1263) | cagcctttgcattatatttt gctgtgttgtctaacaggag | 4043 | | |
| ABCB11 | 50 | intron 14 + 1283 | gctgtgttgtctaacaggag A/C aaagagacacggatttgctc | 4044 | | |
| ABCB11 | 51 | intron 14 + 1339 | tgagatagatatttaggacc G/A tgaccaatttttattttggt | 4045 | | |
| ABCB11 | 52 | intron 14 + 1359 | gtgaccaatttttattttgg T/C tgaaaaatcttatttgaagt | 4046 | | |
| ABCB11 | 53 | intron 14 + 1480 | tattgattagacaataaccc G/A tctggggaagggatatttct | 4047 | | |
| ABCB11 | 54 | intron 15 + 370 | ccttttctaatgtctgcaca G/A cctatttaagaatattccca | 4048 | | |
| ABCB11 | 55 | intron 16 + (550-559) | aaagtttagtgtttctatca (T)9-12 gctacttctgatggacttct | 4049 | | |
| ABCB11 | 56 | intron 17 + 188 | tttctctccccaattcatgg T/G tttttggttagcttctcatc | 4050 | | |
| ABCB11 | 57 | intron 17 + 194 | tccccaattcatgggttttt T/G gttagcttctcatcttcttg | 4051 | | |
| ABCB11 | 58 | intron 17 + (197-198) | caattcatgggtttttggtt (T) agcttctcatcttcttgggg | 4052 | | |
| ABCB11 | 58 | intron 17 + (197-198) | caattcatgggtttttggtt agcttctcatcttcttgggg | 4053 | | |
| ABCB11 | 59 | intron 17 + (289-296) | ggggacttcttttaaaaaaa G/A (A)4 tctgtgtttagtgttcctct | 4054 | | |
| ABCB11 | 60 | intron 17 + 1070 | tcagacttgggttttcctat C/T tttcttcttgagaacaagtt | 4055 | | |
| ABCB11 | 61 | intron 17 + 1651 | tgttaaaatatctcattgta T/C atgctgacggatttttcttg | 4056 | | |
| ABCB11 | 62 | intron 17 + 2226 | ccttaagtctcctcctatca T/A gcaccttgttctcaccagct | 4057 | | |
| ABCB11 | 63 | intron 17 + 2979 | ctctctcttcctttctcagc T/Δ ctactatttcactgttggct | 4058 | | |
| ABCB11 | 64 | intron 17 + 3288 | aatccccatatcctacctta T/G ccatctcatccatgaatctt | 4059 | | |
| ABCB11 | 65 | intron 17 + 3289 | atccccatatcctaccttag C/T catctcatccatgaatcttg | 4060 | | |
| ABCB11 | 66 | intron 18 + 97 | aatatgagttttctaggtat A/G tatctagcagtgtttcaagt | 4061 | | |
| ABCB11 | 67 | intron 18 + 98 | atatgagttttctaggtata T/C atctagcagtgtttcaagtc | 4062 | | |
| ABCB11 | 68 | intron 18 + 892 | ctctgaaagttagtgataca C/T cttatttgtgtttgaatcaa | 4063 | | |
| ABCB11 | 69 | intron 18 + 2681 | atgtatgagatcaagtcagg A/G tcaaatattagacacccata | 4064 | | |
| ABCB11 | 70 | intron 18 + 2681 | ggaccatcctgtggggcaat C/G gttccagaaaatgctggtat | 4065 | | |
| ABCB11 | 71 | intron 18 + 5741 | ctcaccggtataaatacaac C/T gtagcaaaggttttcttttt | 4066 | | |
| ABCB11 | 72 | intron 18 + (5582-5883) | tgcgtattccctcagttcag (C) tttttattcaagccacagca | 4067 | | |
| ABCB11 | 72 | intron 18 + (5582-5883) | tgcgtattccctcagttcag tttttattcaagccacagca | 4068 | | |
| ABCB11 | 73 | intron 19 + 10022 | tggctaagttaaaaaaaaaa A/Δ gagattcaactataattgct | 4069 | | |
| ABCB11 | 74 | intron 21 + 322 | caagattcaatactgccccc C/Δ agggggtgggtgaacagggc | 4070 | | |
| ABCB11 | 75 | intron 22 + 257 | ctgttcaatttcctctcgca T/C agtgattcattccacattcc | 4071 | | |
| ABCB11 | 76 | intron 22 + 552 | taattaatatcttgtccttg G/C ggggtaaatgagggatggta | 4072 | | |
| ABCB11 | 77 | intron 22 + 569 | ttggggggtaaatgagggat G/A gtagcataaacacttctcaa | 4073 | | |
| ABCB11 | 78 | 3′flanking + 243 | aaacaccacagaatgacata G/A aactaaaggcggcaggaatc | 4074 | | |
| ABCC1 | 1 | 5′flanking − 1661 | cattcacccttgggggaccc A/G ggccaataaaaaaatcacag | 4075 | | |
| ABCC1 | 2 | intron 2 + 635 | gatgtgccctacctgaccct T/C ggctcggggcagacttgggg | 4076 | | |
| ABCC1 | 3 | intron 2 + 4769 | gggcaggagtggactcaggg G/Δ ttcctggtccaaatgggttc | 4077 | | |
| ABCC1 | 4 | intron 2 + 10069 | tatggaggttttctcttcct T/C tctgtgagttttctctctga | 4078 | | |
| ABCC1 | 5 | intron 2 + (11965-11984) | taaaagccaatcaaatcaac (T)18-20 aaacaagccacgcatttgcc | 4079 | | |
| ABCC1 | 6 | intron 4 + 4302 | cacctgtaatcccagcacct T/G gggaggccaaggcaagtgga | 4080 | | |
| ABCC1 | 7 | intron 4 + 4394 | gtctttactaaaaatacaaa A/C attagctaggcatggtggcg | 4081 | | |
| ABCC1 | 8 | intron 4 + 4524 | ccactgcgctccagcctggg T/C gacaagagtgaaactctgtc | 4082 | | |
| ABCC1 | 9 | intron 6 + 9045 | aggtccttaaactaccctgc G/A ctccaagaatcagtgcctgg | 4083 | | |
| ABCC1 | 10 | intron 7 + (3059-3071) | agtcttttgtatgcaccact (A)11-13 gccatttttcctgcatgacc | 4084 | | |
| ABCC1 | 11 | intron 8 + (886-889) | ttctatgtaacagtaagaaa GAAA/Δ agcagctgccaattaaacaa | 4085 | | |
| ABCC1 | 12 | intron 11 + 198 | tgaattgtcaggttgatgtt C/A tccttggtggcatggcgttt | 4086 | | |
| ABCC1 | 13 | intron 11 + 784 | tgtggattgatccaggagat C/G aagcaatgttgtcagtactc | 4087 | | |
| ABCC1 | 14 | intron 12 + 122 | agccttgcctgccagttgga C/G tcacttggggagccttaaca | 4088 | | |
| ABCC1 | 15 | intron 12 + (3138-3148) | accccatctctattgaaaag (A)10-12 tcaatataaaaaacatttac | 4089 | | |
| ABCC1 | 16 | intron 12 + 3227 | tggtgatgttgagtgatggg C/T tgatcccagggtcgccccag | 4090 | | |
| ABCC1 | 17 | intron 13 + 2060 | tgctcattacaactattcct T/C cttggtcaggttggcaaatt | 4091 | | |
| ABCC1 | 18 | intron 13 + (2061-2062) | ctcattacaactattccttc (C) ttggtcaggttggcaaatta | 4092 | | |
| ABCC1 | 18 | intron 13 + (2061-2062) | ctcattacaactattccttc ttggtcaggttggcaaatta | 4093 | | |
| ABCC1 | 19 | intron 13 + 11776 | gccacctggggagggcccaa G/A cgcgtctccagggcctgtca | 4094 | | |
| ABCC1 | 20 | intron 14 + 179 | aaagaaagaaaacacatttg A/T cttcttgacagagaactcgc | 4095 | | |
| ABCC1 | 21 | intron 16 + 219 | ctagcacagagggttccctg G/T gattgtaagttacagcagcc | 4096 | | |
| ABCC1 | 22 | intron 16 + 310 | ggaagttctactttcaggtg C/T ggtgtgatccagggactctg | 4097 | | |
| ABCC1 | 23 | intron 16 + 890 | ctctccagagaaaacaatct G/T tagaaggcctgcattgaaaa | 4098 | | |
| ABCC1 | 24 | intron 17 + 1171 | aaccccaggctcaaagaagc G/A tgggaaataatgcatactcc | 4099 | | |
| ABCC1 | 25 | intron 17 + 1332 | cacctctttagtgtctgtgc A/G actgcacatttgtctcttgg | 4100 | | |
| ABCC1 | 26 | exon 18 + 53 | gattcagaatgattctctcc G/A agaaaacatcctttttggat | 4101 | | |
| ABCC1 | 27 | intron 19 + (3373-3379) | ccaagctaggcagtctcaca CA/Δ tgtgcactcacgtggccggg | 4102 | | |
| ABCC1 | 28 | intron 20 + 2730 | gcgtgaggtctgtctctcta C/T ccttccgtccaggtgagcaa | 4103 | | |
| ABCC1 | 29 | intron 20 + 3789 | cttggccccagataggttcc G/C cacccccgcctttctttccc | 4104 | | |
| ABCC1 | 30 | intron 20 + 2919 | gatgcaaatgccgcccacca C/T cctggcacctcgtgcgttca | 4105 | | |
| ABCC1 | 31 | intron 20 + 3024 | cttacatcaaactggggcac G/C cacccccgcctttctttccc | 4106 | | |
| ABCC1 | 32 | intron 20 + 9718 | gtggctgcgctcagtgacga A/C caggagaagtgaaggctgag | 4107 | | |
| ABCC1 | 33 | intron 20 + 9733 | gacgaacaggagaagtgaag G/C ctgaggcttataggagggtg | 4108 | | |
| ABCC1 | 34 | intron 20 + (9895-9896) | gctggttcccagtgtcacac AT/Δ gtgtgtgaggacaggctgca | 4109 | | |
| ABCC1 | 35 | intron 20 + 9952 | ggtatcattcttccttcctg G/A gtgatgtggctatttgtgtt | 4110 | | |
| ABCC1 | 36 | intron 20 + 11120 | gcggagtgggggcagtagtc A/G tcatcatcactgagttattg | 4111 | | |
| ABCC1 | 37 | intron 20 + 11147 | tcactgagttattgtgaacc G/A ggaaagagatatgatctgtg | 4112 | | |
| ABCC1 | 38 | intron 20 + (11629-11631) | tattttgaatatcacttctt CTT/Δ tcaatgcttgggaatcacgg | 4113 | | |
| ABCC1 | 39 | intron 20 + 11864 | gagctccagataccacctgc C/T ccacaaccagacagcctgtt | 4114 | | |
| ABCC1 | 40 | intron 21 + 3860 | tggagagtgacatggtgggg G/Δ tgtggtgcatatattcatat | 4115 | | |
| ABCC1 | 41 | intron 22 + 878 | ttaaagatcgtctattttgg G/A caagtgttaataattctcca | 4116 | | |
| ABCC1 | 42 | intron 22 + (4445-4446) | gtggggctggggctggggct (GGGGCT) gggtgcgtgcatgtgctaag | 4117 | | |
| ABCC1 | 42 | intron 22 + (4445-4446) | gtggggctggggctggggct gggtgcgtgcatgtgctaag | 4118 | | |
| ABCC1 | 43 | intron 23 + 62 | gttgtggctttgtctaatta T/C agaaatggatccttagagtc | 4119 | | |
| ABCC1 | 44 | intron 24 + 3171 | aaccatgaggctcaccatat C/T tcaaaccacgctgcacagct | 4120 | | |
| ABCC1 | 45 | intron 24 + (3349-3368) | ctcttgaaattggtgccagc (T)19-22 ccctgcatttaccaaatatg | 4121 | | |
| ABCC1 | 46 | intron 24 + 3369 | tttttttttttttttttttt T/C ccctgcatttaccaaatatg | 4122 | | |
| ABCC1 | 47 | intron 24 + 3584 | ccaaggatttttatttttca A/G caacaaaggaaatgatttta | 4123 | | |
| ABCC1 | 48 | exon 25 + 60 | gagtcggtcagccgctcccc G/A gtctattcccatttcaacga | 4124 | | |
| ABCC1 | 49 | intron 27 + 4539 | tcttttttactcactgcagt G/A tgaggaacaaatcacattta | 4125 | | |
| ABCC1 | 50 | intron 30 + (1708-1714) | gacccaacactatctcctgg (T)6-7 cttccggtcaagtgtcgggc | 4126 | | |
| ABCC1 | 51 | exon 32 + 652 | tggagaaaatcattttctcc C/T cttggcagtgtcccagggcc | 4127 | | |
| ABCC1 | 52 | 3′flanking + 158 | ctgatgctcttccaggacac G/A aaaagaacccatctttgaat | 4128 | | |
| ABCC1 | 53 | 3′flanking + (187-199) | ccatctttgaatatcaatga (T)11-13 aagtactgttccggggagaa | 4129 | | |
| ABCC1 | 54 | 3′flanking + 2227 | cattagaataggtagtatca G/A ccagccgggcatggtggctc | 4130 | | |
| ABCC2 | 1 | exon1+ 77 | catattaatagaagagtctt C/T gttccagacgcagtccagga | 4131 | | |
| ABCC2 | 2 | intron1+ 413 | gataagttctagaactggca A/C ctaatgatatggactagaag | 4132 | | |
| ABCC2 | 3 | intron2+ 192 | atcaaagtggctttgatttt T/G gcataagaatggtgactctt | 4133 | | |
| ABCC2 | 4 | intron2+ 1020 | agtgctgcgattacaagcct G/C agccacctgcacagcctctg | 4134 | | |
| ABCC2 | 5 | intron2+ 3639 | gtcatatcccacccccaaat C/A gacccaataggtacaatgaa | 4135 | | |
| ABCC2 | 6 | intron2+ 3930 | aaaactggcaggagaatttc A/G ctggagctgcatgcaggact | 4136 | | |
| ABCC2 | 7 | intron2+ 3989 | agtgctgcgattacaagcct G/C agccacctgcacagcctctg | 4137 | | |
| ABCC2 | 8 | intron2+ 4078 | aggtttccagatgtgttccc T/C aggcattcctggtggtagga | 4138 | | |
| ABCC2 | 9 | intron2+ 4171 | cttattctttggtcagttgg C/T tttctaccacctcttagctt | 4139 | | |
| ABCC2 | 10 | intron2+ 4257 | gggtattggaaagttcttgc G/A gctgctggaggctgcggtgt | 4140 | | |
| ABCC2 | 11 | intron2+ 4436 | ggactagtggaagaattaga C/G ctttcctgaataaatagatc | 4141 | | |
| ABCC2 | 12 | intron2+ 5227 | taccataatttatgtgtcct A/G tatgacatgaatttcattgg | 4142 | | |
| ABCC2 | 13 | intron2+ 5373 | gttaaggatatgtgaactca A/G gtgtgtctataggataaatt | 4143 | | |
| ABCC2 | 14 | intron2+ 5538 | ttaatgaggttaagcacatg G/T tcatatgtttaaaagccttt | 4144 | | |
| ABCC2 | 15 | intron3+ 772 | ggtataaggcaagatttttt A/T aaaaaattaattgcttaatc | 4145 | | |
| ABCC2 | 16 | intron3+ 1145 | acatccttctcccctcagtc C/T tcggttagtggcagtattct | 4146 | | |
| ABCC2 | 17 | intron7+ 1658 | ggactcttaccagcttagtt G/T cctggttttctaatctaaaa | 4147 | | |
| ABCC2 | 18 | exon10+ 40 | tggccaggaaggagtacacc G/A ttggagaaacagtgaacctg | 4148 | | |
| ABCC2 | 19 | intron11+ 1672 | aactttttaagtcttaagac T/A ggaaggcctgtgtcctaggc | 4149 | | |
| ABCC2 | 20 | intron12+ 148 | ccctctcaccgccccatgcc A/G cttttcctcctttgtaccat | 4150 | | |
| ABCC2 | 21 | intron13+ 180 | catgagttttctgagcccca G/C tttatctaactataaaatga | 4151 | | |
| ABCC2 | 22 | intron13+ 1497 | gtgcagggtccccctgatgc T/C atagccagttcctctttaga | 4152 | | |
| ABCC2 | 23 | intron15+ 169 | atgagctgaaagcaaaggtt T/C tcagccccttcccctgataa | 4153 | | |
| ABCC2 | 24 | intron15+ 949 | ttccaggtgacacatttagt A/G cctaatttgggaaatgttaa | 4154 | | |
| ABCC2 | 25 | intron16+ 984 | tgttaatctagtccaatccc A/C ttagtaagaaaggaggggtc | 4155 | | |
| ABCC2 | 26 | intron19+ 4059 | catcctgatgcacagttatt C/T aaatttaagctccatttgtt | 4156 | | |
| ABCC2 | 27 | intron19+ 10899 | atgtatggagtatttatgga G/A taaagtattccatgctgtat | 4157 | | |
| ABCC2 | 28 | exon22+ 51 | caagcaataggattgttttc G/A atattcttcatcatccttgc | 4158 | | |
| ABCC2 | 29 | intron23+ 56 | tatactgaggatctttctga C/T agggaggaattattatgtcc | 4159 | | |
| ABCC2 | 30 | intron23+ 432 | tggcagtagagcagggtgag G/A aggattattctgcagaggaa | 4160 | | |
| ABCC2 | 31 | intron23+ 734 | tgagccaactactgtactag G/A cactggggcactcaatgaat | 4161 | | |
| ABCC2 | 32 | intron23+ 801 | atgggccagacccaactcac T/G gattttttagtgtatctgag | 4162 | | |
| ABCC2 | 33 | intron26+ 154 | ctggctccatcttttaccca T/C ggacgtattccttactcttc | 4163 | | |
| ABCC2 | 34 | intron27+ 124 | gggtccctaaagtttccttt C/G ctctaactcaaaggacctaa | 4164 | | |
| ABCC2 | 35 | exon28+ 52 | cagattggcccagcaaaggc A/C agatccagtttaacaactac | 4165 | | |
| ABCC2 | 36 | exon28+ 84 | aacaactaccaagtgcggta C/T cgacctgagctggatctggt | 4166 | | |
| ABCC2 | 37 | exon28+ 129 | agagggatcacttgtgacat C/T ggtagcatggagaaggtagg | 4167 | | |
| ABCC2 | 38 | intron29+ 154 | ttccctaggatggacacgtc A/G tttccagaactttgaaatgt | 4168 | | |
| ABCC2 | 39 | intron30+ 91 | gtgttaggtgatgcctggca T/C agaattttcatccaggtctg | 4169 | | |
| ABCC2 | 40 | intron31+ 170 | gccaaaattttacatcacgc A/G aatgaaaacgaacaaggtta | 4170 | | |
| ABCC2 | 41 | 3′flanking + 371 | gtgaatttttattataagct C/T gttctccttaaaactttatc | 4171 | | |
| ABCC3 | 1 | 5′flanking − 1064 | tccttctgagccccaacaag C/T ggtgctgagttggcgtctgg | 4172 | | |
| ABCC3 | 2 | 5′flanking − (827-820) | ctggggcttcacctgtcctt (C)7-8 aaccctgatcaggctgaagc | 4173 | | |
| ABCC3 | 3 | intron 1 + 1226 | tatttgtacatatatgccct T/G tgtgtgtgtacgcacacacg | 4174 | | |
| ABCC3 | 4 | intron 1 + (1389-1399) | aaacttggggcaatggaggt (A)10-12 ctgtaaaaaggcatatttgg | 4175 | | |
| ABCC3 | 5 | intron 1 + 2070 | gcgcacttctccttgatgct C/T gtgagctatacacacctcct | 4176 | | |
| ABCC3 | 6 | intron 1 + 4477 | gcctgtagtccccagacagg G/A ggttttgccattttggcagg | 4177 | | |
| ABCC3 | 7 | intron 1 + 6189 | agtgaccatgaagtctgcca T/C gagggggcctctgccacgtg | 4178 | | |
| ABCC3 | 8 | intron 2 + 268 | ttgtatttttagtagagatg G/A ggttttgccattttggcagg | 4179 | | |
| ABCC3 | 9 | intron 2 + 376 | tgtgcccagccagcattctg G/C ttttaatgaggccctctccc | 4180 | | |
| ABCC3 | 10 | intron 2 + 446 | ctcacctgacctgcttgggg C/T catgggaatctgacaactga | 4181 | | |
| ABCC3 | 11 | intron 8 + 2323 | gaggctggtggtgagagcgt C/G atcgatagggcgtgcagcag | 4182 | | |
| ABCC3 | 12 | intron 12 + 85 | ctcattggactctaccctga C/Δ accacctccacgctgctcag | 4183 | | |
| ABCC3 | 13 | intron 19 + 1581 | ttcttgttgccctttcaatc C/T ccctcattttattttcatgc | 4184 | | |
| ABCC3 | 14 | exon 22 + 180 | aacacttccctgaggctggg C/T gtctatgctgctttaggaat | 4185 | | |
| ABCC3 | 15 | intron 30 + 1979 | cctctgtctgttccatccct C/G tcctaccctcaccccccact | 4186 | | |
| ABCC3 | 16 | intron 30 + 2340 | atgcaccagccaggcctgaa A/C gaatgagtaagagttggagg | 4187 | | |
| ABCC3 | 17 | 3′flanking + (555-558) | ttttcttgagcaagccaaca AAGA/Δ gtttcttttctgcaggtcag | 4188 | | |
| ABCC3 | 18 | 3′flanking + 1455 | aaccccgtatgattagaact G/A tagtgctgtttaggaagcca | 4189 | | |
| ABCC3 | 19 | 3′flanking + (1650-1659) | aattcacagttaacaaagct (A)9-11 tccttgttataaattacaca | 4190 | | |
| ABCC4 | 1 | 5′flanking − 644 | attcatctgggtcatactct C/T gagttacccggctttcttga | 4191 | | |
| ABCC4 | 2 | exon 1 + 67 | ggagcggagcccgcggccac C/T gccgcctgatcagcgcgacc | 4192 | | |
| ABCC4 | 3 | intron 1 + (864-865) | ctttgaccagcttctttccc CT/Δ gtttccaatactttcacttc | 4193 | | |
| ABCC4 | 4 | intron 1 + 21255 | ggatggaaatggtgagcaca A/G accttggcatttaaggaccg | 4194 | | |
| ABCC4 | 5 | intron 1 + 21503 | ctgttttctacccactgggg T/C cagcaaatcagcccctttta | 4195 | | |
| ABCC4 | 6 | intron 1 + 21900 | tgatgctcaaagcaatacaa C/G tagaaaatataggaggctgg | 4196 | | |
| ABCC4 | 7 | intron 1 + 22005 | aagggggagtcatactccag C/T gtgcattttagtttgtgctt | 4197 | | |
| ABCC4 | 8 | intron 1 + (22256-22264) | tttgtgttgttatttgcgtc (T)8-9 cctggaaggaagtgattggc | 4198 | | |
| ABCC4 | 9 | intron 1 + 27784 | ccagggaactggtggcacac C/G ctgagtctgctaggtgggct | 4199 | | |
| ABCC4 | 10 | intron 1 + 27821 | ggctaaagactcacaacctg A/T gggaaggggccaggaaagaa | 4200 | | |
| ABCC4 | 11 | intron 1 + 27837 | cctgagggaaggggccagga A/G agaaaggaagccatggccta | 4201 | | |
| ABCC4 | 12 | intron 1 + 27880 | gggtgttatttgggacccca C/T gcccatccaggccgacagag | 4202 | | |
| ABCC4 | 13 | intron 1 + 40310 | accaagcaggggaggtgaga A/T ttgtgcagactggggatatt | 4203 | | |
| ABCC4 | 14 | intron 1 + 40372 | ttgcttgaataaaaggatgc G/A agtcactgtattggtgaagt | 4204 | | |
| ABCC4 | 15 | intron 1 + 40413 | ttctttcaaatccaattcct G/A actgatttccttgccttcca | 4205 | | |
| ABCC4 | 16 | intron 1 + 40958 | gaagtttaccgaaaaacaaa A/G caagaaactccccagtaaaa | 4206 | | |
| ABCC4 | 17 | intron 1 + 50060 | tgtggctatggggaacatga G/A gctcatagaaactgaagact | 4207 | | |
| ABCC4 | 18 | intron 2 + 181 | gcctgggggaaactcctgtt G/T cctgtgcctccgtagaggtc | 4208 | | |
| ABCC4 | 19 | intron 2 + 254 | gaggtctgtccctctaggtg G/A aagtgttgtggttggaggag | 4209 | | |
| ABCC4 | 20 | intron 2 + 290 | aggaggttgtctggcttatc T/C gtgctactgatggggcttca | 4210 | | |
| ABCC4 | 21 | intron 2 + 543 | ttacgaagctttttcctcat T/C gtaggttctgggataaagaa | 4211 | | |
| ABCC4 | 22 | intron 3 + 557 | ggccttgcacctgggctggc G/A gtggtgccccagaggctgga | 4212 | | |
| ABCC4 | 23 | intron 3 + 718 | gtgtgtcttccttgttgtcg G/A agtggattgctggttggaag | 4213 | | |
| ABCC4 | 24 | intron 3 + 801 | acattccatgaaaaatcaaa G/A acagccagaagggcaataac | 4214 | | |
| ABCC4 | 25 | intron 3 + 1022 | aggggtggatgttgctgttg T/C tacaaaagggtggctttaaa | 4215 | | |
| ABCC4 | 26 | intron 3 + 1471 | tgctggggtgtcccagcgat A/G gtgtttccacatggccccga | 4216 | | |
| ABCC4 | 27 | intron 3 + 1490 | tagtgtttccacatggcccc G/A atcagtttcagttggaaaga | 4217 | | |
| ABCC4 | 28 | intron 3 + (1833-1834) | gggctgccagccacttgggg (G) tggggtctctaacccacaga | 4218 | | |
| ABCC4 | 28 | intron 3 + (1833-1834) | gggctgccagccacttgggg tggggtctctaacccacaga | 4219 | | |
| ABCC4 | 29 | intron 3 + 1870 | cagatggtgactggactaca G/A tgagatttgggtaagctttt | 4220 | | |
| ABCC4 | 30 | intron 3 + 1927 | gaagtagaggctgtagaagc G/A tgaatttctcctgagacttg | 4221 | | |
| ABCC4 | 31 | intron 3 + 1970 | gacaggccccactctggtgc A/T aggagcatggtaatctttac | 4222 | | |
| ABCC4 | 32 | intron 3 + 2039 | gatcgaggggagctttaata T/C gggtacagttggtggagagc | 4223 | | |
| ABCC4 | 33 | intron 3 + (2067-2068) | ttggtggagagctggtcttt (CTTT) tagcggggtggttattgggc | 4224 | | |
| ABCC4 | 33 | intron 3 + (2067-2068) | ttggtggagagctggtcttt tagcgggggtggttattgggc | 4225 | | |
| ABCC4 | 34 | intron 3 + 3563 | cattgactgatggtctgggc G/a gatgtcaagttccctgtttt | 4226 | | |
| ABCC4 | 35 | intron 3 + 3696 | tgcttggcaaggatgaagac C/G ccagatgagtcactagtatg | 4227 | | |
| ABCC4 | 36 | intron 3 + 4093 | aagtaatccttggatttttt T/C ttttcttttccttctagcag | 4228 | | |
| ABCC4 | 37 | intron 3 + 4097 | aatccttggatttttttttt T/Δ cttttccttctagacagtgaa | 4229 | | |
| ABCC4 | 38 | intron 3 + 9724 | aaaaaccagcattactcacc A/G atgagcccatttgcttgact | 4230 | | |
| ABCC4 | 39 | intron 3 + 9988 | aaaggcaaagagcactgagc G/A tctggctgatagcccaggtg | 4231 | | |
| ABCC4 | 40 | intron 3 + 10952 | gttaaaattgcattccctac A/G tcttgttcagaaggtaagcc | 4232 | | |
| ABCC4 | 41 | intron 3 + 11125 | gctcaatttctgctgtgttt A/G atttttgactccacactacc | 4233 | | |
| ABCC4 | 42 | intron 3 + 11244 | ccaagagcctggaatcctcc C/Δ aagtctggttcttttcccca | 4234 | | |
| ABCC4 | 43 | intron 3 + 11916 | gtcttgaccaaaaaaaaaaa A/Δ tttagctctacatgatggtg | 4235 | | |
| ABCC4 | 44 | intron 3 + 12047 | actatactccagcatgggtg A/G cagagcaagcaatatctgaa | 4236 | | |
| ABCC4 | 45 | exon 4 + 205 | tgaggttacgagtagccatg T/G gccatatgatttatcggaag | 4237 | | |
| ABCC4 | 46 | intron 4 + (412-414) | ttatggaaatttttgttgtt GTT/Δ cattaaaaccttcacttaca | 4238 | | |
| ABCC4 | 47 | intron 4 + (9757-9756) | tgacatctgtcatttttttt (T) cctgctgcacaaatctcttc | 4239 | | |
| ABCC4 | 47 | intron 4 + (9757-9756) | tgacatctgtcatttttttt cctgctgcacaaatctcttc | 4240 | | |
| ABCC4 | 48 | intron 4 + 6373 | atgttttgttctagatagta C/G agttttcttgtaatctcaaa | 4241 | | |
| ABCC4 | 49 | intron 4 + 6267 | acttccaccattcacagtat T/C gttcttaatggcatgcggat | 4242 | | |
| ABCC4 | 50 | intron 4 + 6095 | agatccttcatttcctaggg T/C gtacaaatttcaaggctttt | 4243 | | |
| ABCC4 | 51 | intron 4 + 6057 | ttgctatgctagattgattt C/T ctccccaagagttgttaatt | 4244 | | |
| ABCC4 | 52 | intron 4 + 5295 | agttgtctggcttacagtag A/G tgcttactaaatggtagctt | 4245 | | |
| ABCC4 | 53 | intron 4 + 803 | agcttcacctgtttcagccc C/T gcttccatgagcttcacctg | 4246 | | |
| ABCC4 | 54 | intron 4 + 736 | attcagcagcctccacatcc C/T ccttctccgtacttctgtcc | 4247 | | |
| ABCC4 | 55 | intron 4 + 728 | gcctccacatcctccttctc C/T gtacttctgtcctagctagg | 4248 | | |
| ABCC4 | 56 | intron 4 + 624 | ccacccagtgtccctcagtt A/C gaactgtccccagttctctg | 4249 | | |
| ABCC4 | 57 | intron 4 + 470 | ttgatactccatatttgtca C/T ttcccattgaacacattgaa | 4250 | | |
| ABCC4 | 58 | intron 4 + 411 | ggtgaagagactaaggcccc G/A tgtgtttaataatgttgcac | 4251 | | |
| ABCC4 | 59 | intron 4 + 323 | tgttcctctgacagcctctc C/T gttcttccctaatttggctc | 4252 | | |
| ABCC4 | 60 | intron 4 + 246 | gtccttttgtacttgggggc A/G tgtccaaattcattaaatga | 4253 | | |
| ABCC4 | 61 | intron 4 + 199 | agatttttcttcttcctacc C/T ctcgctttgctgtcctgaca | 4254 | | |
| ABCC4 | 62 | intron 5 + 73 | ccttttattctttctggagg C/T aggggctcactctgttcaca | 4255 | | |
| ABCC4 | 63 | intron 5 + 403 | aagggatcacgccttgttgc G/A caggctggtctcaagattct | 4256 | | |
| ABCC4 | 64 | intron 5 + 937 | ccagaatggcttcacctgtg G/C tgggtgcttggctttctgct | 4257 | | |
| ABCC4 | 65 | intron 6 + 150 | ggctcagccaagggggcctc C/T gtccttatgctgaaggcaaa | 4258 | | |
| ABCC4 | 66 | intron 6 + (380-381) | tgtgttagagctgttttcac (AT) gtgtatatatgtgtgttatt | 4259 | | |
| ABCC4 | 66 | intron 7 + (380-381) | tgtgttagagctgttttcac gtgtatatatgtgtgttatt | 4260 | | |
| ABCC4 | 67 | intron 8 + 894 | tttgttgttgttgcccagga A/T ggtctcaaactcctgggttc | 4261 | | |
| ABCC4 | 68 | intron 8 + 82 | tatttagcatcactatgttc C/G agtgtaatgacatttaactc | 4262 | | |
| ABCC4 | 69 | intron 8 + 100 | tccagtgtaatgacatttaa C/T tctctcataaccaaaacgtg | 4263 | | |
| ABCC4 | 70 | intron 8 + 5212 | tcagggaattgtggtccaat A/T tgcagctagggaagaaatcc | 4264 | | |
| ABCC4 | 71 | intron 8 + 5444 | gaaaccttaatttcccctca T/G gtacatagtttctggtggga | 4265 | | |
| ABCC4 | 72 | intron 8 + 8969 | tcaccctcctgagtgactag A/G gaaagtccagctagcccctc | 4266 | | |
| ABCC4 | 73 | intron 8 + 9106 | ccagtgctcaataggtttac T/C gtgtgcatagtttttatttt | 4267 | | |
| ABCC4 | 74 | intron 8 + 9412 | tgtttgtaagtgcaggatgg G/A ggacacatctctgccctgta | 4268 | | |
| ABCC4 | 75 | intron 9 + 116 | tggcttgcttatttactgaa A/G ctatgttacaaagattctca | 4269 | | |
| ABCC4 | 76 | intron 9 + 1384 | cacggcaggaagctgcaccc T/C ggggctggagatgatgtctg | 4270 | | |
| ABCC4 | 77 | intron 9 + 1459 | agatttgggagcagagggcg A/G gggtctcttctgagggtact | 4271 | | |
| ABCC4 | 78 | intron 9 + 1632 | agcagcactcctgcccagcc C/A cactgcctccgtcctcccct | 4272 | | |
| ABCC4 | 79 | intron 9 + 3630 | gtaaatttttcattttgaag G/Δ ttatcttgatctcttattcc | 4273 | | |
| ABCC4 | 80 | intron 9 + 3830 | ggtgttccacccttcaggga C/T gccagattcattttgaagaa | 4274 | | |
| ABCC4 | 81 | intron 9 + 3940 | gagcatttaccaaagtgtgt C/T gtgcagaagaatagccactt | 4275 | | |
| ABCC4 | 82 | intron 10 + 1504 | gggcaaggctgcattgcagt G/A gcttattcttgtctcgagtg | 4276 | | |
| ABCC4 | 83 | intron 11 + 1817 | ttttagggagttgagaaaca G/C atggcaaattttgctagttt | 4277 | | |
| ABCC4 | 84 | intron 11 + 3342 | actggaattattctggcttg T/C aggtacagagattgcatgtg | 4278 | | |
| ABCC4 | 85 | intron 11 + 3377 | catgtgtaatcaaaacctgc T/C ggacagaaatggtcctgagc | 4279 | | |
| ABCC4 | 86 | intron 11 + (3610-3625) | tcctggccaccctcccccgc (A)15-17 gtcctagaggaaaaaatagg | 4280 | | |
| ABCC4 | 87 | intron 11 + 3737 | ataagttcatcgagctaaaa A/G tatatttgagataaaataat | 4281 | | |
| ABCC4 | 88 | intron 11 + 6953 | agagtagagacaaagaaatg C/A caccttgatctgtaagaggg | 4282 | | |
| ABCC4 | 89 | intron 13 + 442 | ctatgacaggttagaagtga G/C gtccttgggaccaacatagg | 4283 | | |
| ABCC4 | 90 | intron 13 + 449 | tgacgtccttgggaccaaca T/C agggctttcttgggaaggct | 4284 | | |
| ABCC4 | 91 | intron 13 + 633 | tgaacacttaaaacccacag G/A catgtaggcctggcttgcct | 4285 | | |
| ABCC4 | 92 | intron 13 + 645 | acccacaggcatgtaggcct G/T gcttgcctttgaaactagtt | 4286 | | |
| ABCC4 | 93 | intron 13 + 3306 | aatgttctcaacgagttaga A/C aattggattgaacaatatgc | 4287 | | |
| ABCC4 | 94 | intron 14 + 252 | taatttagaactttttgttt A/G cctcttccatgacttaattc | 4288 | | |
| ABCC4 | 95 | intron 15 + 124 | tggattctgtggtttcaggg C/T tctattccatgatattggta | 4289 | | |
| ABCC4 | 96 | intron 15 + 1552 | tttggacttctgcctgtttc C/T ccacagctttgtcaacagag | 4290 | | |
| ABCC4 | 97 | intron 16 + 157 | cctactggtgttccatgtcc G/A ttacaaagacctgcgaaaaa | 4291 | | |
| ABCC4 | 98 | intron 17 + 329 | cccaaattgtggttcatttt T/C aaaaaaatgtatttatctaa | 4292 | | |
| ABCC4 | 99 | exon 18 + 56 | atggaggaggaaatgtaacc G/A agaagctagatcttaactgg | 4293 | | |
| ABCC4 | 100 | intron 19 + 7202 | aattaaaaataatgtttttt T/Δ cacataacaatggttatatg | 4294 | | |
| ABCC4 | 101 | intron 19 + 7445 | ttttggcataatttttaatc T/C actagaatgttctgattcat | 4295 | | |
| ABCC4 | 102 | intron 19 + 9018 | tacgtgatggcctgaagaga A/G aaaccgtacattggttcttt | 4296 | | |
| ABCC4 | 103 | intron 19 + 11388 | aagagttcagagattttggg A/G gttggaggaaaaaatagcat | 4297 | | |
| ABCC4 | 104 | intron 19 + 11646 | cattatttttaatttttttt T/Δ cctcctgttggtgtcagaat | 4298 | | |
| ABCC4 | 105 | intron 19 + 13517 | gagaaacttacattattttt A/T aaaaatgctataactagtcc | 4299 | | |
| ABCC4 | 106 | intron 19 + 21033 | tgggagtgccctgggctagc G/A ctgaaacttcaggttttcag | 4300 | | |
| ABCC4 | 107 | intron 19 + 21095 | agacttttggaagaagcaga A/T ctgaaggtaagactgagtaa | 4301 | | |
| ABCC4 | 108 | intron 19 + 21634 | gtgctatttctgagcactca C/T ggccccattgggcatgggct | 4302 | | |
| ABCC4 | 109 | intron 19 + 21715 | tgttttgctcaccccctaca C/T agcttgccctcatgcttctc | 4303 | | |
| ABCC4 | 110 | intron 19 + 23090 | agcaacagacttggagactt G/A agcttctaaaagtttcatta | 4304 | | |
| ABCC4 | 111 | intron 19 + 24297 | cgaatgtgatgaatgtggga A/G cctttttgagatagcagcac | 4305 | | |
| ABCC4 | 112 | intron 19 + 25947 | gagtctaaattaaatatgag C/A aaaactagaaaccatttaaa | 4306 | | |
| ABCC4 | 113 | intron 19 + 30193 | acagatttgcaagagtctac A/C aaagtgataatattctgtca | 4307 | | |
| ABCC4 | 114 | intron 19 + 36938 | aagccgagtcaatctcttgg C/G tatcttctgtggactacttt | 4308 | | |
| ABCC4 | 115 | intron 19 + 37322 | gttcccatgagggctgaccc C/T gcctcacccctggtaaccgc | 4309 | | |
| ABCC4 | 116 | intron 19 + (38361-38362) | cggggttagcttccctagct (T) gcggagggtttctgagaaaa | 4310 | | |
| ABCC4 | 116 | intron 19 + (38361-38362) | cggggttagcttccctagct gcggagggtttctgagaaaa | 4311 | | |
| ABCC4 | 117 | intron 19 + 38746 | taaagacatgctggtaatta T/C gtaaaataaagataagtcaa | 4312 | | |
| ABCC4 | 118 | intron 19 + 42343 | tgtaagggcagaatcagcag C/T aacgattggatgttcccgga | 4313 | | |
| ABCC4 | 119 | intron 19 + 44733 | agcaggctggggaaaaaaaa A/Δ tacagaggttatcattatgt | 4314 | | |
| ABCC4 | 120 | intron 20 + (405-419) | aagggcaaatacttaggcac (T)13-15 ggatagaaccaggtgtggtt | 4315 | | |
| ABCC4 | 121 | intron 20 + (637-648) | ctaagtcttaagctgtcttt (A)12-13 ccaacaatcctacagaaata | 4316 | | |
| ABCC4 | 122 | intron 20 + 842 | caagctggggcacttttttt T/Δ tcccaagtgtttattttgga | 4317 | | |
| ABCC4 | 123 | intron 20 + 843 | aagctggggcactttttttt T/C cccaagtgtttattttggaa | 4318 | | |
| ABCC4 | 124 | intron 20 + 1347 | ggacctctgatttttttttt T/Δ cttttgcaaacatttttaaa | 4319 | | |
| ABCC4 | 125 | intron 20 + (14553-14567) | tccctacacacccctcatct (A)13-15 tcagcagcttgactgagctt | 4320 | | |
| ABCC4 | 126 | intron 20 + 15487 | ggttttttccagtgtgatag C/T acatgtagaaagcagtactg | 4321 | | |
| ABCC4 | 127 | intron 20 + 16161 | gcgttgagtcatgaagccga T/C agtgccgcttgtgcatcgca | 4322 | | |
| ABCC4 | 128 | intron 20 + 30891 | acgtcccccactgttctatc C/T ttctcaagaagcaagcgttg | 4323 | | |
| ABCC4 | 129 | intron 20 + 31180 | ccttgcacgtgctcatacat G/A tcatttgctattgttatcat | 4324 | | |
| ABCC4 | 130 | intron 20 + 31283 | gtgttaaagctaaaaaaaaa A/Δ ccctgttagacattttgact | 4325 | | |
| ABCC4 | 131 | intron 21 + 4204 | ttgaccctgccctgaaaccc A/T gttggagataaaacagtggc | 4326 | | |
| ABCC4 | 132 | intron 22 + 1026 | gtgccctactccacgtaaaa A/C tcttctgtagctcaactgag | 4327 | | |
| ABCC4 | 133 | intron 23 + 377 | gcctggtgcatgaggttgag A/G aaaattctcagcaggagagt | 4328 | | |
| ABCC4 | 134 | intron 25 + 4122 | cccttttgattaaaattgca C/G/T tgggacaagaaccaccccca | 4329 | | |
| ABCC4 | 135 | intron 25 + 6418 | ttgcactgaggtaatggctg C/A agaaattaaagtgagggtat | 4330 | | |
| ABCC4 | 136 | intron 25 + (8765-8775) | tgcatcctgtgatttttttc (T)5-11 aatcctgccgcctggatctc | 4331 | | |
| ABCC4 | 137 | intron 26 + 67 | tatgtttaattgcttttact G/C ttattgctttttttaattgg | 4332 | | |
| ABCC4 | 138 | intron 26 + (101-109) | taattggatgaaaggattgt (T)8-9 cacccaatagagcatgtttt | 4333 | | |
| ABCC4 | 139 | intron 28 + 391 | tagatatgatcttttttttt T/Δ aaatctctattgtgaagtag | 4334 | | |
| ABCC4 | 140 | intron 29 + 2569 | atcctcttttttctaatacg C/T accactatctccacattaaa | 4335 | | |
| ABCC4 | 141 | intron 29 + 7820 | gaaaaacaacctgtgtcctg C/T ttggaggttcagcatattct | 4336 | | |
| ABCC4 | 142 | intron 30 + 6269 | tagatgttctttgggcattg A/G aaagatggtgttatctgttt | 4337 | | |
| ABCC4 | 143 | intron 30 + 6320 | gtttaataaggtttaattag C/T tctactttgttaattacatt | 4338 | | |
| ABCC4 | 144 | intron 30 + 6474 | ctttgatgctatggttttca A/G tccacagatgttcataactt | 4339 | | |
| ABCC4 | 145 | intron 30 + 6519 | ttccactatgaattatattt C/T ctgccattttaacacacctt | 4340 | | |
| ABCC4 | 146 | intron 30 + 6574 | aatggttttggtcctaaatc C/T acactggttcaaaactagac | 4341 | | |
| ABCC4 | 147 | intron 30 + 6680 | aggtgtgtctcctgtatatg A/G cgtggttaggttttactctg | 4342 | | |
| ABCC4 | 148 | intron 30 − 704 | acgtttatcagaaaacctgt A/C tctcttctagttcagctaga | 4343 | | |
| ABCC4 | 149 | intron 30 − 228 | atctatgaatcagagtgatc A/G gaactaaaatggatctacag | 4344 | | |
| ABCC4 | 150 | intron 30 − (14-5) | acattctttttatgcttacc (T)9-10 ctaggtatacttcaaaagaa | 4345 | | |
| ABCC4 | 151 | exon 31 + 146 | agtccgttccgaaggcattt G/T ccactagtttttggactatg | 4346 | | |
| ABCC4 | 152 | 3′flanking + 173 | atttttaaaggagtaggaca A/G agttgtcacaggtttttgtt | 4347 | | |
| ABCC4 | 153 | 3′flanking + (430-440) | tgtaccccttactccccatc (A)10-11 tggatacatggttaaaggat | 4348 | | |
| ABCC4 | 154 | 3′flanking + 556 | aaaggtgctttgatactgaa G/A gacacaaatgtgaccgtcca | 4349 | | |
| ABCC4 | 155 | 3′flanking + 1144 | cctccctgaaattgcatata T/C gtatatagacatgcacacgt | 4350 | | |
| ABCC4 | 156 | 3′flanking + 1426 | tttaggtgactgaaattgca A/T cagtgatcataatgaggttt | 4351 | | |
| ABCC5 | 1 | intron 1 + 628 | ttctgccacacagagccgcg G/C gtggctttgtgtttatcaca | 4352 | | |
| ABCC5 | 2 | intron 1 + 1834 | tgagttccagtgacctcctc C/T gtttcaaactgctcaccgcc | 4353 | | |
| ABCC5 | 3 | intron 1 + 3055 | agaaagtctttaaaaaaaaa A/Δ ccaacctttctattgtatac | 4354 | | |
| ABCC5 | 4 | intron 2 − 20280 | gaatgcatcgctactaagta T/C ttttgtaagttcagacacca | 4355 | | |
| ABCC5 | 5 | intron 2 − 20260 | tttttgtaagttcagacacc A/T tctagaatctgcttgaccgt | 4356 | | |
| ABCC5 | 6 | intron 2 − 19204 | tgaaataaagcattcgcaca C/T ctacccactttcttcgggac | 4357 | | |
| ABCC5 | 7 | intron 2 − 19043 | agaaagtctttaaaaaaaaa A/Δ ccaacctttctattgtatac | 4358 | | |
| ABCC5 | 8 | intron 2 − 18824 | gaatgcatcgctactaagta T/C ttttgtaagttcagacacca | 4359 | | |
| ABCC5 | 9 | intron 2 − 18807 | tttttgtaagttcagacacc A/T tctagaatctgcttgaccgt | 4360 | | |
| ABCC5 | 10 | intron 2 − (18735-18734) | atagaagcttaaactcacaa (A) cacgtactctacatagatga | 4361 | | |
| ABCC5 | 10 | intron 2 − (18735-18734) | atagaagcttaaactcacaa cacgtactctacatagatga | 4362 | | |
| ABCC5 | 11 | intron 2 − 15903 | taccaaagcctgctcatgga G/A gtagaaagcaagactgacat | 4363 | | |
| ABCC5 | 12 | intron 2 − 15901 | ccaaagcctgctcatggagg C/T agaaagcaagactgacatgt | 4364 | | |
| ABCC5 | 13 | intron 2 − 15847 | tggatggaacctcaaaggcc G/A tcttgcccagtccccattta | 4365 | | |
| ABCC5 | 14 | intron 2 − 15605 | aggagacgccacgacactga C/T agctgtacctgacctgaggg | 4366 | | |
| ABCC5 | 15 | intron 2 − 13571 | ccgattgtgccccagatacc G/A ctttatttgaggggtgtgcc | 4367 | | |
| ABCC5 | 16 | intron 2 − 13402 | taccctgctgttgtccggcc G/T ccaggaagggattggattgt | 4368 | | |
| ABCC5 | 17 | intron 2 − 13325 | cccagaggcctccgtgcagg G/C gaaaagcccttggttgccct | 4369 | | |
| ABCC5 | 18 | intron 2 − 7293 | tttgttaggataaaattgca C/T tgagtgcctgttctaaacca | 4370 | | |
| ABCC5 | 19 | intron 5 + 374 | ccgggctggtgagccagcac C/T gggaacataccaagtgcctg | 4371 | | |
| ABCC5 | 20 | intron 5 + (2212-2213) | cgcctcctgcagtgctctct CT/Δ tggtgaatgctaactctgct | 4372 | | |
| ABCC5 | 21 | intron 5 + 3283 | acccagagagagtctgggtt C/T tggaattcagcgtagctacc | 4373 | | |
| ABCC5 | 22 | intron 5 + 3469 | ttggctttcttttgttgtgg C/T tttttgttttatttttgtca | 4374 | | |
| ABCC5 | 23 | intron 7 + 443 | cacttttattaaagacagta C/T gattacataacatttggccc | 4375 | | |
| ABCC5 | 24 | intron 7 + 458 | cagtacgattacataacatt T/G ggcccatcctagcaagcagg | 4376 | | |
| ABCC5 | 25 | intron 9 + 176 | caaaacaaaacaaacaaaca A/G acaaaaaaaaaataccacat | 4377 | | |
| ABCC5 | 26 | intron 9 + 214 | catatggagatgatgctgtg G/T tcctctccttactggacctg | 4378 | | |
| ABCC5 | 27 | intron 10 + 703 | tgtgggctggaattccttga T/C gttgccactgcatagattag | 4379 | | |
| ABCC5 | 28 | intron 10 + 3580 | catggggctggagctgtgaa A/G accagtaggtactggcatgt | 4380 | | |
| ABCC5 | 29 | intron 10 + 3655 | atcctttgaataactcttta G/A gggagagaaatgatggaaat | 4381 | | |
| ABCC5 | 30 | intron 10 + 3854 | gaagtttagaatcatgacac T/C tcggggaagataggatcagg | 4382 | | |
| ABCC5 | 31 | intron 10 + 5040 | ctttgaagacatgagagttt C/T ttggcaagaagatgttctct | 4383 | | |
| ABCC5 | 32 | intron 10 + 5316 | cagttaaatgtcattaggtc C/T gctttaggctggctgagggg | 4384 | | |
| ABCC5 | 33 | intron 12 + 234 | tgactgttgtcccagctgga G/A ccatttggtctcatgccttc | 4385 | | |
| ABCC5 | 34 | intron 12 + 300 | tgccacaggtatgcccgtgt A/G ttgaaaatgtcagagataag | 4386 | | |
| ABCC5 | 35 | intron 12 + 318 | gtattgaaaatgtcagagat A/G agagatgagcagacacccta | 4387 | | |
| ABCC5 | 36 | intron 12 + 1545 | gtagcatccctaaaccaaga C/T aaatgtctactatcagtccc | 4388 | | |
| ABCC5 | 37 | intron 13 + 20 | ggcaaggaatgtttggcttc T/C gtcatgctttccatcttggc | 4389 | | |
| ABCC5 | 38 | intron 14 + 278 | ttctatccagatatttttaa A/G actacaagtaagcgtgtgca | 4390 | | |
| ABCC5 | 39 | intron 16 + 1663 | tgactggagacttttttttt T/Δ aaatattattagatcaattc | 4391 | | |
| ABCC5 | 40 | intron 16 + 1664 | gactggagactttttttttt A/T aatattattagatcaattca | 4392 | | |
| ABCC5 | 41 | intron 17 + 20 | ggtaatggccttttttgaaa T/G ttttagatttgtcatcaaag | 4393 | | |
| ABCC5 | 42 | intron 18 + 232 | ggacacctgcaggctatctg C/T tctcatccgttgtgtattag | 4394 | | |
| ABCC5 | 43 | intron 19 + 249 | ggaccagtaggaacagagcc G/A tccctgggccctgaccactc | 4395 | | |
| ABCC5 | 44 | intron 20 + 846 | ttaaccagaagaaaaaaggc G/A gtggggtggggagacagcca | 4396 | | |
| ABCC5 | 45 | intron 20 + 1154 | tcttgagacgaaaaaaaaaa A/Δ tcagagcatccaggtttcta | 4397 | | |
| ABCC5 | 46 | intron 22 + (1424-1425) | gaggaaatgcagcggaatat (AT) caactctggttttaacaggg | 4398 | | |
| ABCC5 | 46 | intron 22 + (1424-1425) | gaggaaatgcagcggaatat caactctggttttaacaggg | 4399 | | |
| ABCC5 | 47 | intron 24 + 132 | atcccacagaatctccagca A/G tctctcaaccgtgcttggaa | 4400 | | |
| ABCC5 | 48 | intron 24 − 874 | gtgctggagaggttaggatt A/G cggtcagtggtggtacaaag | 4401 | | |
| ABCC5 | 49 | intron 24 − 630 | tgatgataaaaattacccaa G/A cagttatatcacagcatttt | 4402 | | |
| ABCC5 | 50 | intron 24 − 102 | acagggtggcagctacctct G/C tgtggctactatggttgtcc | 4403 | | |
| ABCC5 | 51 | exon 25 + 120 | taccgagaaaacctccctct C/T gtcctaaagaaagtatcctt | 4404 | | |
| ABCC5 | 52 | intron 26 + 263 | ctgggcccagggctctgctc C/T gtgacttcggacaagttatt | 4405 | | |
| ABCC5 | 53 | intron 26 − 3257 | ccgagggtgaattgctgtgt T/C gtctcacactttgggagata | 4406 | | |
| ABCC5 | 54 | intron 27 + 873 | gttttttcctctgctctatc G/A ggattcttctcatttgaaga | 4407 | | |
| ABCC5 | 55 | intron 29 + (2733-2734) | gtgtccaaaggaaggacacg (TGTCCAAAGGAAGGACACG) | 4408 | | |
| | | | cttatgttctccttgtggcc | | | |
| ABCC5 | 55 | intron 29 + (2733-2734) | gtgtccaaaggaaggacacg cttatgttctccttgtggcc | 4409 | | |
| ABCC5 | 56 | intron 29 + 2959 | acatgattttccacggctac A/G tagaagtccatcataggaat | 4410 | | |
| ABCC5 | 57 | intron 29 + 4020 | aataaaaaaataagggggga G/A gtgcacgcagggctagttga | 4411 | | |
| ABCC5 | 58 | exon 30 + 684 | cccctctgccgcctccccac G/A gccgctccaggggtggctgg | 4412 | | |
| ABCC5 | 59 | exon 30 + 947 | agtctatccacagagagtcc C/T actgcctcaggttcctatgg | 4413 | | |
| ABCC5 | 60 | exon 30 + (1145-1160) | tcaccgcagtcgtcgcacag (TC)6-8 ccctcaaagtctgcaacttt | 4414 | | |
| ABCC5 | 61 | 3′flanking + 4 | attattttggattttgtaaa A/C ctcttcgtgtatcaaacaat | 4415 | | |
| ABCC5 | 62 | 3′flanking + 2008 | cccgcagacctggcacagcc C/Δ tgttctcaaaggggagctcc | 4416 | | |
| ABCC5 | 63 | 3′flanking + 2052 | cccagctaggacaggccagc A/G ccaggcagttaggaccgtgg | 4417 | | |
| ABCC7 | 1 | 5′flanking − 834 | gctaaaacactccaaagcct T/G ccttaaaaatgcgcactggg | 4418 | | |
| ABCC7 | 2 | 5′flanking − 729 | cctccttgcagatttttttt T/Δ ctctttcagtacgtgtccta | 4419 | | |
| ABCC7 | 3 | exon 1 + 125 | tagcagggaccccagcgccc G/C agagaccatgcagaggtcgc | 4420 | | |
| ABCC7 | 4 | intron 1 + 6200 | ctatgtgagacgttaagaag G/A tagaggtggccaagaaggaa | 4421 | | |
| ABCC7 | 5 | intron 1 + 7538 | agttctctttcttagcatgg C/A ctacagaggtgcaactacct | 4422 | | |
| ABCC7 | 6 | intron 1 + 13519 | gaaacttaaatcttgagtca T/C acaattgtgtctacatactg | 4423 | | |
| ABCC7 | 7 | intron 1 + 14110 | attacacagtattttttttt T/Δ aattttggggaaagtcgatt | 4424 | | |
| ABCC7 | 8 | intron 1 + 14293 | gccaggcagattcctgactc C/Δ tataacccagagcttatcag | 4425 | | |
| ABCC7 | 9 | intron 1 + 14316 | taacccagagcttatcagag C/G atttatgtccccaaagagaa | 4426 | | |
| ABCC7 | 10 | intron 1 + 14433 | cagaataacaatgatggctc G/A gaaaaatatgggtatttctg | 4427 | | |
| ABCC7 | 11 | intron 1 + 14824 | acgttttgacagttgcacaa G/C tttctttctttaagctttaa | 4428 | | |
| ABCC7 | 12 | intron 1 + 23401 | aatatttttgaaaatcacta C/G ggtatcctgcatagtgattt | 4429 | | |
| ABCC7 | 13 | intron 3 + 879 | gaaaaatttcagttcataca C/A ccccatgaaaaatacattta | 4430 | | |
| ABCC7 | 14 | intron 3 + 922 | acttatcttaacaaagatga G/C tacacttaggcccagaatgt | 4431 | | |
| ABCC7 | 15 | intron 3 + 933 | caaagatgagtacacttagg C/T ccagaatgttctctaatgct | 4432 | | |
| ABCC7 | 16 | intron 3 + 13704 | tttttccaaataaaaaaaaa A/Δ tcaggtgatatctgtaaatg | 4433 | | |
| ABCC7 | 17 | intron 3 + 13758 | tattaaagaacatgatgctt A/C aaacagattagggaaaacta | 4434 | | |
| ABCC7 | 18 | intron 4 + 240 | ctctgttgtagttttttttt T/Δ ctcctaatcatgttatcatt | 4435 | | |
| ABCC7 | 19 | intron 4 + 376 | ttatgttcagcaagaagagt A/G taatatatgattgttaatga | 4436 | | |
| ABCC7 | 20 | intron 4 + 586 | tgtccagacaagagaccaaa T/C tgccgaggcatcatttaggt | 4437 | | |
| ABCC7 | 21 | intron 4 + 1089 | tttcaatctgaacattttac G/A taagtgaagactttgttaga | 4438 | | |
| ABCC7 | 22 | intron 4 + 1615 | aaagttaggtggtattgtat C/T tgtcttcctttctcaatgtt | 4439 | | |
| ABCC7 | 23 | intron 4 + 1946 | aatacaaacaaacttgagct T/C tgcctatacttttcaagaat | 4440 | | |
| ABCC7 | 24 | intron 6 + 783 | tatctaagttttggagtcaa A/G tagcactttgtttgaatccc | 4441 | | |
| ABCC7 | 25 | intron 6 + (1104-1131) | gattgattgattgattgatt (GATT)6-7 tacagagatcagagagctgg | 4442 | | |
| ABCC7 | 26 | intron 7 + (731-732) | gtagcaatgagaccattttt (T) cttcagttgagctccatgtt | 4443 | | |
| ABCC7 | 26 | intron 7 + (731-732) | gtagcaatgagaccattttt cttcagttgagctccatgtt | 4444 | | |
| ABCC7 | 27 | intron 7 + 1434 | gaatgtttggttgtaacctg T/C ataatctggcatgaaattgt | 4445 | | |
| ABCC7 | 28 | intron 8 + 752 | catgctctcttctcagtccc A/G ttccttcattatatcaccta | 4446 | | |
| ABCC7 | 29 | intron 8 + 1109 | tatggccaagacttcagtat G/A cgtggacttaattcttcctt | 4447 | | |
| ABCC7 | 30 | intron 8 + 1312 | atgaagacattcattttttt T/Δ ctccgtccaatgttggatta | 4448 | | |
| ABCC7 | 31 | intron 9 + (6521-6522) | gtgtgtgtgtgtgtgtgtgt (GT) ttttttaacagggatttggg | 4449 | | |
| ABCC7 | 31 | intron 9 + (6521-6522) | gtgtgtgtgtgtgtgtgtgt ttttttaacagggatttggg | 4450 | | |
| ABCC7 | 32 | intron 10 + 2119 | gaacactttatagttttttt T/G ggacaaaagatctagctaaa | 4451 | | |
| ABCC7 | 33 | intron 11 + 3867 | tttttcttcaagaaattaga A/Δ gaggggagaaattggtttaa | 4452 | | |
| ABCC7 | 34 | intron 11 + 11844 | tgaatcaaaatcatctaaaa A/Δ gctttcagaaaccagacttt | 4453 | | |
| ABCC7 | 35 | intron 11 + 12144 | atattaaacagagttacata T/C acttacaacttcatacatat | 4454 | | |
| ABCC7 | 36 | intron 11 + 20975 | gtgtggatagtaaatgccag G/A gtaaatcacatagcatctaa | 4455 | | |
| ABCC7 | 37 | intron 11 + 27057 | atggaagagaagttttagta G/A aggggaggaaggaggaggtg | 4456 | | |
| ABCC7 | 38 | intron 11 + 27131 | gagagagacttttttttttt T/Δ aaggcgagagtttactacct | 4457 | | |
| ABCC7 | 39 | intron 13 + 152 | gtattaactcaaatctgatc T/A gccctactgggccaggattc | 4458 | | |
| ABCC7 | 40 | intron 13 + 287 | tttgcagtatcattgccttg T/C gatatatattactttaatta | 4459 | | |
| ABCC7 | 41 | intron 15 + (85-86) | atacatatatatgcacacac AT/Δ aaatatgtatatatacacat | 4460 | | |
| ABCC7 | 42 | intron 15 + 106 | taaatatgtatatatacaca T/A gtatacatgtataagtatgc | 4461 | | |
| ABCC7 | 43 | intron 15 + 3341 | ggaagtataaatttgtaaat A/C actgagacccaaacttacaa | 4462 | | |
| ABCC7 | 44 | intron 15 + 5556 | tgctattgactaatagtaat A/T attttagggcagctttatga | 4463 | | |
| ABCC7 | 45 | intron 15 + 5919 | tggtagttctatgtggaaac C/A gtgaggaaataattttatat | 4464 | | |
| ABCC7 | 46 | intron 17 + 2479 | caaaaaggtatggaagtcag A/C ggagaaggagacccctatgt | 4465 | | |
| ABCC7 | 47 | intron 18 − 81 | aagtatgcaaaaaaaaaaaa A/Δ gaaataaatcactgacacac | 4466 | | |
| ABCC7 | 48 | intron 19 + 751 | cattaataaaataacaaatc A/G tatctattcaaagaatggca | 4467 | | |
| ABCC7 | 49 | intron 19 + 820 | tgacatttgtgatatgatta T/C tctaatttagtctttttcag | 4468 | | |
| ABCC7 | 50 | intron 21 + 1532 | ttacctttaacttttttttt T/Δ atgccttttactttctctat | 4469 | | |
| ABCC7 | 51 | intron 21 + 1607 | atgcttttggagttgggtct C/T ataaatgtatagaaatgttt | 4470 | | |
| ABCC7 | 52 | intron 21 + 11260 | atgtggaacaatcatgacta T/C atgccttttactttctctat | 4471 | | |
| ABCC7 | 53 | intron 22 + (130-131) | agaatcaatattaaacacac AT/Δ gttttattatatggagtcat | 4472 | | |
| ABCC7 | 54 | intron 23 + 1837 | ctgtcctaaagtttaaaaag A/Δ aaaaaaaaaggaagaaggaa | 4473 | | |
| ABCC7 | 55 | intron 24 + (7100-7112) | cctttacaaactcttagaca (T)12-14 agtttaacatgttacaaaac | 4474 | | |
| ABCC7 | 56 | intron 25 + 237 | actcttcccccttgtcaaca C/T atgatgaagcttttaaatac | 4475 | | |
| ABCC7 | 57 | exon 27 + 115 | gggtgaagctctttccccac C/T ggaactcaagcaagtgcaag | 4476 | | |
| ABCC7 | 58 | exon 27 + 334 | ggatgaattaagtttttttt T/Δ aaaaaagaaacatttggtaa | 4477 | | |
| ABCC8 | 1 | 5′flanking − 1099 | aaaggggctgaaggggtctt T/C cttttgtgttcccctgactg | 4478 | | |
| ABCC8 | 2 | 5′flanking − (424-422) | caccccaccaccaccaccac CAC/Δ aaggtaacgttctgccccac | 4479 | | |
| ABCC8 | 3 | intron 1 + 1212 | agcctgggcaacatagtgag A/G ccccccccgccctttctaca | 4480 | | |
| ABCC8 | 4 | intron 2 + 1003 | aggaggactgtgaatcccag C/A ctgcatgtttgggtcggatt | 4481 | | |
| ABCC8 | 5 | intron 2 + 1253 | catctcactaaggaagaatc C/T agtaaccagcaaggatgaga | 4482 | | |
| ABCC8 | 6 | intron 2 + 1382 | cccagactgcactcctgcag T/C gctgcctggctcctgtagtt | 4483 | | |
| ABCC8 | 7 | intron 2 + 2371 | tttcagagctgtctggaaat T/A tagggggcaggtgggagggg | 4484 | | |
| ABCC8 | 8 | intron 3 + 1957 | ccctacccctagcccagggg C/T ccccacatgagtatgaatgg | 4485 | | |
| ABCC8 | 9 | intron 3 + (2088-2089) | agagaacccttcattaacca (CCA) gggcgtggctgaccagtgtc | 4486 | | |
| ABCC8 | 9 | intron 3 + (2088-2089) | agagaacccttcattaacca gggcgtggctgaccagtgtc | 4487 | | |
| ABCC8 | 10 | intron 3 + 2204 | taaagcacaagttatcaccc G/A tggatggatttgtccttttc | 4488 | | |
| ABCC8 | 11 | intron 3 + 2286 | ttatctccccttgaaaggac A/G ctccacagagccagaaattc | 4489 | | |
| ABCC8 | 12 | intron 3 + 2312 | cagagccagaaattctagaa C/G agggaaaagtggaggggagg | 4490 | | |
| ABCC8 | 13 | intron 3 + 2356 | ctgtgaactgcagggacaga A/G ggaaatgggtattgggagaa | 4491 | | |
| ABCC8 | 14 | intron 3 + 2359 | tgaactgcagggacagaagg A/C aatgggtattgggagaatgg | 4492 | | |
| ABCC8 | 15 | intron 3 + 2370 | gacagaaggaaatgggtatt G/A ggagaatggccagccctcca | 4493 | | |
| ABCC8 | 16 | intron 3 + 2382 | tgggtattgggagaatggcc A/G gccctccaaggggctgatgt | 4494 | | |
| ABCC8 | 17 | intron 3 + 4910 | ggggacagccttcagctgtg G/A aattcctccagtcctagaga | 4495 | | |
| ABCC8 | 18 | intron 3 + 4969 | cattattccagtcctgaggc A/G tgagagcagaaggccgatgc | 4496 | | |
| ABCC8 | 19 | intron 3 + 5003 | ccgatgcttctgccctccat C/G ctaatgtcctcctgcaggga | 4497 | | |
| ABCC8 | 20 | intron 3 + 5019 | ccatcctaatgtcctcctgc A/C gggacccaaggtggatggca | 4498 | | |
| ABCC8 | 21 | intron 4 + 14 | ggtgagggtaagcaggccac C/T tgggccagggtggggtggga | 4499 | | |
| ABCC8 | 22 | intron 4 + 187 | agacactgcatctggcccac G/A tgtgctctaccccagggtcc | 4500 | | |
| ABCC8 | 23 | intron 4 + 204 | cacgtgtgctctaccccagg G/C tcccagagggagaggggggt | 4501 | | |
| ABCC8 | 24 | intron 4 + 254 | gttcgctgaggttggcggat G/A actttccgtagaaagggaag | 4502 | | |
| ABCC8 | 25 | intron 4 + 357 | tgtattcatatcgtcacgct G/C gtaaatgaatgagtaagtgt | 4503 | | |
| ABCC8 | 26 | intron 5 + 92 | ggcattaggtcaaaatcctg G/A tgggacaaaaggggaaactg | 4504 | | |
| ABCC8 | 27 | intron 6 + 4205 | tctgtagaaagtacatgggg C/A catgaagatcattggcttga | 4505 | | |
| ABCC8 | 28 | intron 6 + 5519 | gattcccagggaatgttaaa A/C aggaccgggtcttcctaaac | 4506 | | |
| ABCC8 | 29 | intron 6 + 5575 | tctgacccagtaccagccag G/C ggggcaagtttccatccccc | 4507 | | |
| ABCC8 | 30 | intron 6 + 6587 | gttgccatctgagatcttgc C/T ggaagtacacaagagaccct | 4508 | | |
| ABCC8 | 31 | intron 6 + 6747 | ttccactggccttttctgct C/T agtaattgctacattacagg | 4509 | | |
| ABCC8 | 32 | intron 9 + 191 | gaggaagctgcctcccggtg A/G ggacaggaagcgggcatggc | 4510 | | |
| ABCC8 | 33 | intron 10 + 1963 | cccaggagtccaacctccct T/G tgtccagctagaccatggtg | 4511 | | |
| ABCC8 | 34 | intron 10 + 2724 | cctgggacatgttttcttat A/G taaacagcatcaaaagatgt | 4512 | | |
| ABCC8 | 35 | intron 10 + 2938 | gcccgcccaggactcctcac G/C tgtccaagtcacctagggag | 4513 | | |
| ABCC8 | 36 | intron 10 + 3094 | tccgaggatgtgtttttttt T/Δ ccctccgttagtcagcagtg | 4514 | | |
| ABCC8 | 37 | intron 10 + 3368 | tcctgctcatatgcggcacc A/G tcagacttctgggcaggcaa | 4515 | | |
| ABCC8 | 38 | intron 10 + 8897 | ggtattgattaaaagcctca C/T gggcagagaaattcgccatc | 4516 | | |
| ABCC8 | 39 | intron 11 + 308 | tgtgtattgtagaagtgatg G/A gaaatccagaacagaaagct | 4517 | | |
| ABCC8 | 40 | intron 11 + 1171 | gccctctcatttcccttcca G/A tgctgagcgtttccagtgtg | 4518 | | |
| ABCC8 | 41 | exon 12 + 7 | gcctctgtccacagactttc G/A tgggccacgtcagcttcttc | 4519 | | |
| ABCC8 | 42 | intron 12 + 356 | accaagaatgaggccatccc G/T tccccacgtggctgccccat | 4520 | | |
| ABCC8 | 43 | intron 12 + 934 | tgggttcaaagatggaatgg G/T gcataactcagcaaaattat | 4521 | | |
| ABCC8 | 44 | intron 12 + 1370 | gggagggaggctggacaggg C/G atgaaggcagagcctggtgg | 4522 | | |
| ABCC8 | 45 | intron 15 + 412 | ggaggtgggacccaggatgg C/T gtttcttgggaccacaagga | 4523 | | |
| ABCC8 | 46 | intron 15 + 688 | actcccccggccccactcac A/G tctgccaccttccctccctg | 4524 | | |
| ABCC8 | 47 | intron 16 + 4464 | actcattccaagtattgatc G/A agaagagaggtaggtactgg | 4525 | | |
| ABCC8 | 48 | intron 16 + 4574 | ttgaagatcttaagttgttt T/C ggagagcttactgtgtgctg | 4526 | | |
| ABCC8 | 49 | intron 16 + 5011 | tgcacttgccgcctacttat T/G ccagacccaatgattgggtc | 4527 | | |
| ABCC8 | 50 | intron 16 + 7608 | tatagattaatgaggctctg A/G gtccctcaaaaccttccctc | 4528 | | |
| ABCC8 | 51 | intron 16 + 7730 | cccttacctctccaaaaaac A/G cttgagataccctagaggtg | 4529 | | |
| ABCC8 | 52 | intron 16 + 8369 | ttgcaaactgagttagggcc T/C ggagagcttactgtgtgctg | 4530 | | |
| ABCC8 | 53 | intron 16 + 9708 | tgcacttgccgcctacttat T/G ccagacccaatgattgggtc | 4531 | | |
| ABCC8 | 54 | intron 17 + 651 | tatagattaatgaggctctg A/G gtccctcaaaaccttccctc | 4532 | | |
| ABCC8 | 55 | intron 17 + 692 | cccttacctctccaaaaaac A/G cttgagataccctagaggtg | 4533 | | |
| ABCC8 | 56 | intron 17 + 1541 | ctcaggatcttcctggagga C/T atggttcactcccatgagag | 4534 | | |
| ABCC8 | 57 | intron 18 + 580 | actaagcagatttctaccaa C/T tgcacctccccatccccttg | 4535 | | |
| ABCC8 | 58 | intron 18 + 658 | gaacaagcccctgagaatgc C/T ttccgcaccccctactcccg | 4536 | | |
| ABCC8 | 59 | intron 18 + 660 | acaagcccctgagaatgcct T/C ccgcaccccctactcccgcc | 4537 | | |
| ABCC8 | 60 | intron 19 + 93 | gcccttccatcgatcaccca T/C acccagccatctcactcccc | 4538 | | |
| ABCC8 | 61 | intron 19 + 123 | tctcactccccaggtgctta T/C ctgcactccagcctctccat | 4539 | | |
| ABCC8 | 62 | intron 19 + 219 | cataggggagagggcaggaa C/T ggagggaagggagagagccc | 4540 | | |
| ABCC8 | 63 | intron 19 + 845 | tagtatttaacctgcccaaa C/T gctgtgtgaagtgctgacct | 4541 | | |
| ABCC8 | 64 | intron 20 + 338 | tcccctccacaagcttagac A/G aacaggattctcctgtgact | 4542 | | |
| ABCC8 | 65 | exon 21 + 10 | tttggtgacagggcatcaac C/T tgtctggtggtcaacgccag | 4543 | | |
| ABCC8 | 66 | intron 21 + 192 | caaggatagcacaaatgacc C/Δ attgcagacttcagatggag | 4544 | | |
| ABCC8 | 67 | intron 23 + 17 | gaaggtgggtatatccaggg A/G tggccaagcagccacccctg | 4545 | | |
| ABCC8 | 68 | intron 23 + 67 | gttctgctagaacctgaact C/T ataaaggtcttcctgtcctt | 4546 | | |
| ABCC8 | 69 | intron 26 + 268 | gtgagcgtctgcacatccaa G/C taaagattgttttctcctcc | 4547 | | |
| ABCC8 | 70 | intron 26 + 308 | cgataagtgggtgtaatttg C/T ccatccccacccatgagttc | 4548 | | |
| ABCC8 | 71 | intron 26 + 348 | cagctccctgccctcccctc A/G ctctctctccctcagccagc | 4549 | | |
| ABCC8 | 72 | intron 26 + 807 | gacagctgctgagtcaggcc G/A agccggcagctgagaaaggc | 4550 | | |
| ABCC8 | 73 | intron 26 + 834 | cagctgagaaaggcggcagt G/C gtcagatgggcttgagaaac | 4551 | | |
| ABCC8 | 74 | intron 28 + (118-121) | cctccaaaaaataaaaacaa AAAA/Δ cagaaatgaaggaaatagaa | 4552 | | |
| ABCC8 | 75 | intron 28 + 1348 | tggggtaagcggaagacggg G/A ttgaacgctttgagtttggt | 4553 | | |
| ABCC8 | 76 | intron 29 + 1253 | ctcttagggatcttgtctaa G/T taaagaagagcagagcaaag | 4554 | | |
| ABCC8 | 77 | intron 29 + 1589 | cagatcccagcttcctgtaa A/G cagcctcagatcaggccaaa | 4555 | | |
| ABCC8 | 78 | intron 29 + 2322 | gcgcctcacactcctataac G/A cgcacatgccctgatgcaca | 4556 | | |
| ABCC8 | 79 | intron 29 + 2348 | atgccctgatgcacacacat T/C ttcaacacgcacttactcta | 4557 | | |
| ABCC8 | 80 | intron 29 + 2418 | agacacgtcaccctcccaca C/T gtctccaccctgggggtgtg | 4558 | | |
| ABCC8 | 81 | intron 29 + 2494 | tcagtcccctcagacacatg C/A cctctctccacgcagagaca | 4559 | | |
| ABCC8 | 82 | intron 29 + 2735 | gcggccaaggagagtgatga C/T ggcagcccaggttgatcaga | 4560 | | |
| ABCC8 | 83 | intron 30 + 386 | gctcctggggctccagcctt C/T gcagcccttgtgtgtgtctg | 4561 | | |
| ABCC8 | 84 | intron 33 + 93 | ggcttcgcagtcacctcgtg G/T ccctccagggccgaggcctc | 4562 | | |
| ABCC8 | 85 | intron 33 + 358 | agggacctgggggcagacag C/T gaggccacccttgtattgag | 4563 | | |
| ABCC8 | 86 | intron 38 + 54 | cccagggacaggactggcct G/C ttgtggccgtcatcagtgca | 4564 | | |
| ABCC8 | 87 | intron 38 + 466 | aggacattctggccacatgc C/Δ tcatcctcctcctccaagcc | 4565 | | |
| ABCC8 | 88 | intron 38 + 529 | tggcccccaccgcgggtggt A/G ttcccaccatcctgacccgc | 4566 | | |
| ABCC9 | 1 | intron 3 + 38 | tgttgtttctccttaaagag C/A tatttgtttttccccccaaa | 4567 | | |
| ABCC9 | 2 | intron 3 + 305 | gctggccttctggcttgcag T/A agttgtattttaagaatcag | 4568 | | |
| ABCC9 | 3 | intron 3 + 320 | tgcagaagttgtattttaag A/G atcagagctcttgtgaggag | 4569 | | |
| ABCC9 | 4 | intron 3 + 631 | ttctgtggaaatcagaggct G/C tctaaaatattcctaatttt | 4570 | | |
| ABCC9 | 5 | intron 3 + 8644 | tggacgcactcaacattttc A/G agttattactccttcaactc | 4571 | | |
| ABCC9 | 6 | intron 4 + 757 | aggatatcatgaaacactga A/C tcttagtaaaaactatcttt | 4572 | | |
| ABCD3 | 16 | (intron 9 1493) | tcatcttcttccataggctt A/G ggtgtggagaggagatagaa | 4643 | | |
| ABCD3 | 17 | (intron 13 1534) | tctgttgagttggggatcct A/G tggaaacctcttccttcatc | 4644 | | |
| ABCD3 | 18 | (intron 16 4310) | gaaaagtgaatgctgagtag G/T ttagccaggcttgatttaga | 4645 | | |
| ABCD3 | 19 | (intron 20 273) | ttctaaaagttcagagaaac T/A ctgtagctcattattcctgg | 4646 | | |
| ABCD3 | 20 | (intron 20 1664) | ctcaaaagaaaaaaaaaaaa A/C aaaaaacacatgatccataa | 4647 | | |
| ABCD3 | 21 | (intron 20 6693) | cttaaggtttgtgttttact C/T tgagcaattagtatttccca | 4648 | | |
| ABCD3 | 22 | (intron 21 7171) | atcataaacagagaaataat A/G tcttaaatgagctctgaaaa | 4649 | | |
| ABCD3 | 23 | (intron 22 1220) | ctagaaatcaaaggcattta A/G aatatagccaagcctttatg | 4650 | | |
| ABCD3 | 24 | (intron 22 1358) | agtagcaaaataatcatcac G/A ccagtgatcatgtgaaggag | 4651 | | |
| ABCD3 | 25 | (intron 4 4448-4461) | taactttcttgtagttagcg (T)11-14 aactgttttactttttaggg | 4652 | | |
| ABCD3 | 26 | (intron 5 268) | gttttttggcattttttttt T/Δ aaccttcagtccaggttttc | 4653 | | |
| ABCD3 | 27 | (intron 5 891-902) | ttggtgtaaaacctgtagtg (T)10-13 aacaaatgcaaatatagtgt | 4654 | | |
| ABCD3 | 28 | (intron 7 1226-1227) | gggaatggggggtgtatcta (T) tacaactttccatgtaattt | 4655 | | |
| ABCD3 | 28 | (intron 7 1226-1227) | gggaatggggggtgtatcta tacaactttccatgtaattt | 4656 | | |
| ABCD3 | 29 | (intron 8 1129) | cagatttacttttttttttt T/Δ aatcttgaatacttactagc | 4657 | | |
| ABCD3 | 30 | (intron 13 1595-1596) | tgaaacataataaagcacac (TA) gttatcattaatactttatg | 4658 | | |
| ABCD3 | 30 | (intron 13 1595-1596) | tgaaacataataaagcacac gttatcattaatactttatg | 4659 | | |
| ABCD3 | 31 | (intron 16 7337-7351) | ttaattacttcacagactga (T)13-15 caggttcgatctggggctaa | 4660 | | |
| ABCD3 | 32 | (intron 18 12) | gttcctcaggtaagacctag C/Δ ttgagttatctttgatctaa | 4661 | | |
| ABCD3 | 33 | (intron 20 1652-1670) | gcaagactctgtctcaaaag (A)17-20 cacatgatccataatagagg | 4662 | | |
| ABCD3 | 34 | (intron 20 2262-2273) | ttaatccatttttgtaaatc (T)11-13 accttaaattagcaactatc | 4663 | | |
| ABCD3 | 35 | (3′untranslated region | taaaataaagttgagcttag (T)8-9 aaaaaaaaaacaaagcaaca | 4664 | | |
| | | 2072-2079) | | | | |
| ABCD3 | 36 | (3′untranslated region | gttgagcttagttttttttt (A)10-11 caaagcaacaaattaactag | 4665 | | |
| | | 2080-2091) | | | | |
| ABCD3 | 37 | (3′untranslated region | acttattttctgttcagatt (A)16-19 ctcagatatcctatacaacc | 4666 | | |
| | | 3349-3368) | | | | |
| ABCD4 | 1 | (intron 1 276) | tggcattctttttttgaaaa G/A aagaacctcaggtgcacaaa | 4667 | | |
| ABCD4 | 2 | (intron 1 329) | cttctcagttcttgacaccc T/C gtgggccaatgcaaggctcc | 4668 | | |
| ABCD4 | 3 | (intron 3 171) | ttaagcacgttgatcttgct A/G ttggcccacgtgggactgat | 4669 | | |
| ABCD4 | 4 | (intron 3 449) | cctacccctcattcagtagg G/A gggctaccacctgctcactc | 4670 | | |
| ABCD4 | 5 | (intron 5 273) | gacaggggctacctgagagg G/T aacaggagtcagggctgagg | 4671 | | |
| ABCD4 | 6 | (intron 7 240) | tagtcttagtggcctagcgt G/A gggcctgaaattgtcaaatg | 4672 | | |
| ABCD4 | 7 | (intron 7 267) | gaaattgtcaaatgaatgaa T/C gcctcatcctcttgctggtg | 4673 | | |
| ABCD4 | 8 | (coding region 910 | tctatggagacctgagtccc G/A cagagcttagcaccctggtc | 4674 | | |
| | | (Ala 304 Thr)) | | | | |
| ABCD4 | 9 | (coding region 981 | atcagctgcttcacccagct C/A atcgacctgtccacgacgct | 4675 | | |
| | | (Leu 327 Leu)) | | | | |
| ABCD4 | 10 | (coding region 1102 | gcgagatcctgggcgagagc G/A agtggggcttggacacgtga | 4676 | | |
| | | (Glu 367 Lys)) | | | | |
| ABCD4 | 11 | (intron 13 191) | tggattgggcccactactca T/C agcagctcctgaggcaggta | 4677 | | |
| ABCD4 | 12 | (intron 13 262) | acgcgtatgtcaaacaccca A/G ggtcggattctggggcccct | 4678 | | |
| ABCD4 | 13 | (intron 17 848) | cctctgctcctctggcccat C/G cttctccctgaggcagggct | 4679 | | |
| ABCD4 | 14 | (intron 17 946) | gtgggaggagaagcagcggc G/A gcagagggcagggctttgat | 4680 | | |
| ABCD4 | 15 | (intron 18 41) | ggcctgaggaggagaaagaa C/T ccaaaggctcagcctggcca | 4681 | | |
| ABCD4 | 16 | (3′untranslated region 2 | gcccaggtctaggtttctgt G/A ggggacactgaatctcccag | 4682 | | |
| AGCG1 | 1 | (5′flanking region − 386) | gcaataatcattggctagag G/A tattgtgatatgatgtcatt | 4683 | | |
| AGCG1 | 2 | (intron 1 199) | caccaaatattggtgagctg C/T ctggatttgggagatgcagt | 4684 | | |
| AGCG1 | 3 | (intron 1 291) | acttggggtccggtgtgagg A/C tcctgcactcggtttctgtg | 4685 | | |
| AGCG1 | 4 | (intron 1 318) | actcggtttctgtgatggtg T/A gtgcaggggagtcacaagtt | 4686 | | |
| AGCG1 | 5 | (intron 1 468) | ggtcccaacgggtttctaga T/C ccctccagagaagcctttgg | 4687 | | |
| AGCG1 | 6 | (intron 2 434) | ctgggtacaggttttgttcc G/A gttggtctgctattgagtat | 4688 | | |
| AGCG1 | 7 | (intron 3 1839) | ttaaaatgagttgtttttct C/G ctaaagcctttagggagttg | 4689 | | |
| AGCG1 | 8 | (intron 3 3076) | tttgtcacttccttcgtctc C/T ggctctacttccctgggggt | 4690 | | |
| AGCG1 | 9 | (intron 3 3352) | gttccttggaggaaacgtgg G/A gtacacagtggttccagtta | 4691 | | |
| AGCG1 | 10 | (intron 3 8030) | acagtgaagcacaaggcagc C/T gaagacacagcaggcaggtc | 4692 | | |
| AGCG1 | 11 | (intron 3 8066) | aggtcaggtctgtgtgcaca T/C tggcaggctgc a/g tgcagacc | 4693 | | |
| AGCG1 | 12 | (intron 3 8092) | ggctgc a/g tgcagaccagcct C/T ggcccaggtggagaagcaga | 4694 | | |
| AGCG1 | 13 | (intron 3 8285) | ctggacatgtgactcccctg C/T acccaccctcacaagcacca | 4695 | | |
| AGCG1 | 14 | (intron 3 8860) | cagggtgatagggagtccaa T/C tggacacaggttcagtttgc | 4696 | | |
| AGCG1 | 15 | (intron 4 2319) | gggggtgaacagagggcaga G/A gcctgggcatcttcactcag | 4697 | | |
| AGCG1 | 16 | (intron 4 2557) | gaagggaagaagcagcagca A/G gaaagaagccccctggccct | 4698 | | |
| AGCG1 | 17 | (intron 5 139) | tgacccagggcaccctagag T/A ggcgcccggctccgatcgct | 4699 | | |
| AGCG1 | 18 | (intron 5 177) | gctgcccctgcccctccgcc A/C gggccacctggagcctcggg | 4700 | | |
| AGCG1 | 19 | (intron 6 13) | cagttactgtaagtgctgtt T/C ccaggggtggtca g/a gaatct | 4701 | | |
| AGCG1 | 20 | (intron 6 27) | gctgtt t/c ccaggggtggtca G/A gaatctccctttctggtttt | 4702 | | |
| AGCG1 | 21 | (intron 6 1191) | gctaagcagagttaggcccc G/A gctagtccttgaatgagaga | 4703 | | |
| AGCG1 | 22 | (intron 6 1449) | atgctggagcccctgagttc G/A gtgggcatacaaggggtggc | 4704 | | |
| AGCG1 | 23 | (intron 6 2282) | ctcgcatcacgcagttttca C/T gatcctattaattgggtgag | 4705 | | |
| AGCG1 | 24 | (intron 6 3853) | cctgggcttcagcaggggcc T/C cacacctgcaatgggtg c/t ct | 4706 | | |
| ABCG1 | 25 | (intron 6 381) | cc t/c cacacctgcaatgggtg C/T ctggggagagggtgcagatg | 4707 | | |
| ABCG1 | 26 | (intron 6 4175) | tccaaagcccagatttggtg T/C ttttggggctcttttggaat | 4708 | | |
| ABCG1 | 27 | (intron 7 4) | ctggtggaggaagaaaggta G/A ggagggcggctgctttgtgt | 4709 | | |
| ABCG1 | 28 | (intron 7 576) | agctcaggaggtgtctggaa C/T gccacacagtgcaggagttt | 4710 | | |
| ABCG1 | 29 | (intron 7 1426) | aattctccttctcaacttaa A/G gaaatattttatagaaaaat | 4711 | | |
| ABCG1 | 30 | (intron 7 2342) | agagcctgcaatgggccgcc G/A agggacctgcccatgactca | 4712 | | |
| ABCG1 | 31 | (intron 7 2399) | gaggggttgacagacaggat A/G tgtctg c/g tgtgttccagctg | 4713 | | |
| ABCG1 | 32 | (intron 7 2406) | tgacagacaggat a/g tgtctg C/G tgtgttccagctgctggttt | 4714 | | |
| ABCG1 | 33 | (intron 7 2911) | ccctctctgtgcccactgtt G/C tcccaacaccagcctgttct | 4715 | | |
| ABCG1 | 34 | (intron 7 4363) | tataatagattcctagcaga A/G aacataattgtgagaggaac | 4716 | | |
| ABCG1 | 35 | (intron 7 4752) | gctttcagagcccattcaca C/T aagggtctcattttattagg | 4717 | | |
| ABCG1 | 36 | (intron 7 5026) | ccaggtctgtgggatttcag G/A ccaaaaaggagcgtagcaag | 4718 | | |
| ABCG1 | 37 | (intron 7 5532) | gggttaaatattccgggcag C/T gccaagtcagattatctgta | 4719 | | |
| ABCG1 | 38 | (intron 7 5681) | gctaaagtgcatggaaggca T/C catgaataaatcctttcagg | 4720 | | |
| ABCG1 | 39 | (intron 7 9243) | gcctgagagcgctggcagta G/A gaagggtcgccagtgtggac | 4721 | | |
| ABCG1 | 40 | (intron 7 11371) | gggctctcttggagcccttt T/G tctctcccagccctgcgtct | 4722 | | |
| ABCG1 | 41 | (intron 7 12420) | gggatttcgaatctcaacac T/C ctgagctctgtgctttcccc | 4723 | | |
| ABCG1 | 42 | (intron 7 12985) | ctattggcaggtcgtgaaca T/C tgttcttggatttgcaaata | 4724 | | |
| ABCG1 | 43 | (intron 7 20041) | acatggccggcttcccttct T/C cctc g/a gaatggcctggaatt | 4725 | | |
| ABCG1 | 44 | (intron 7 20046) | gccggcttcccttct t/c cctc G/A gaatggcctggaattcgatc | 4726 | | |
| ABCG1 | 45 | (intron 7 21058) | acaagacttagaatttgacc G/A tgattttaaaactattctaa | 4727 | | |
| ABCG1 | 46 | (intron 7 26189) | ttcttggatgtggccatgca C/T gggggcaagggtttgatgag | 4728 | | |
| ABCG1 | 47 | (intron 7 27453) | atcatgtggtttgggggaaa G/C ctgggaccccacttggtaca | 4729 | | |
| ABCG1 | 48 | (intron 7 29810) | attgtttctcctggttttgt T/C tgtgttgactttccctttaa | 4730 | | |
| ABCG1 | 49 | (intron 10 2116) | aaacagggcttgagtcctcc G/A taagggacaggagaccttcc | 4731 | | |
| ABCG1 | 50 | (intron 13 1196) | tgaaaagaaaatggatgagt G/A gaa a/c ccaaaagagagaaaat | 4732 | | |
| ABCG1 | 51 | (intron 13 1200) | aagaaaatggatgagt g/a gaa A/C ccaaaagagagaaaatgtgg | 4733 | | |
| ABCG1 | 52 | (intron 13 2041) | aagcagaggcttttccaccc G/A gagactcaagaagctgctcc | 4734 | | |
| ABCG1 | 53 | (intron 13 2490) | gtggtgaagtagagctgagc A/T cacgggggagccctccatcc | 4735 | | |
| ABCG1 | 54 | (intron 13 2822) | cagcaggctccgtgctgaag T/C cacagcaagccaggcccttg | 4736 | | |
| ABCG1 | 55 | (intron 13 2850) | agccaggcccttggcctgcc G/A gagctggaagacccagaaca | 4737 | | |
| ABCG1 | 56 | (intron 13 2919) | gcctcccaggagtagctaca C/T gggacccgaaggcagatggc | 4738 | | |
| ABCG1 | 57 | (intron 13 3506) | ggcagcctgggctgccgaga T/C cctccctggagcgcccgccg | 4739 | | |
| ABCG1 | 58 | (intron 13 3538) | cgcccgccgggaagccccag G/A ggggctggagctaca a/g gtgg | 4740 | | |
| ABCG1 | 59 | (intron 13 3554) | ccag g/a ggggctggagctaca A/G gtggccttgcaggttttttg | 4741 | | |
| ABCG1 | 60 | (intron 13 3721) | ccagctcatgggcaggggtg C/T ggagggaaaggcacccacag | 4742 | | |
| ABCG1 | 61 | (intron 13 3921) | gaagaccagcagtcgatgcc A/G gctgggaagagggctctgcc | 4743 | | |
| ABCG1 | 62 | (intron 13 3979) | acccaccagccttttccaga C/T agccttccagaagctgtttc | 4744 | | |
| ABCG1 | 63 | (intron 13 4291) | gagccgctggagtagggtcc G/A cttgctatggctcccagggg | 4745 | | |
| ABCG1 | 64 | (intron 13 4968) | tattgactggacaccttctc C/T gtatggggcactgggctagg | 4746 | | |
| ABCG1 | 65 | (intron 16 672) | atcagtaacgggtcactaac G/A gatgctgctgagtggggcag | 4747 | | |
| ABCG1 | 66 | (intron 16 891) | tggcccactgttgagggtgt G/A ggtgaccagaggggcctgga | 4748 | | |
| ABCG1 | 67 | (intron 18 1616) | ctggaggagaagacaggata A/C agtctaagacgtg c/t tgtcac | 4749 | | |
| ABCG1 | 68 | (intron 18 1630) | aggata a/c agtctaagacgtg C/T tgtcacagagttcagggtcc | 4750 | | |
| ABCG1 | 69 | (intron 18 1674) | gcttccaaaggccgcatccg G/T gttgttctctgagc c/t gagga | 4751 | | |
| ABCG1 | 70 | (intron 18 1689) | atccg g/t gttgttctctgagc C/T gaggacggctttgcgaacgc | 4752 | | |
| ABCG1 | 71 | (intron 19 446) | tggctgacagtgaacacagc G/A gctgcttctccagaacttta | 4753 | | |
| ABCG1 | 72 | (intron 22 243) | acccggagagccatggcagg A/C ccaagtgttctggacgttgc | 4754 | | |
| ABCG1 | 73 | (3′flanking region 1257) | atggggcccacagccctgcc T/C cagaagcagctttggtctcg | 4755 | | |
| ABCG1 | 74 | (3′flanking region 1438) | gggggaagagcttgggaacc A/G tgagggctgttaggctgcaa | 4756 | | |
| ABCG1 | 75 | (3′flanking region 1518) | tgaagggtgaactggagtag G/C tgaggattctgcagttgacg | 4757 | | |
| ABCG1 | 76 | (intron 3 3754-3755) | ctccaccctgcacctccctg (G) cctccttgatttccctcatc | 4758 | | |
| ABCG1 | 76 | (intron 3 3754-3755) | ctccaccctgcacctccctg cctccttgatttccctcatc | 4759 | | |
| ABCG1 | 77 | (intron 3 7848-7854) | cagtttccagaatttggggg (A)6-7 tcccataagctgtcatactt | 4760 | | |
| ABCG1 | 78 | (intron 4 190-191) | tgtcgagagctccccttgcc (C) tggttgatcctcagggttct | 4761 | | |
| ABCG1 | 78 | (intron 4 190-191) | tgtcgagagctccccttgcc tggttgatcctcagggttct | 4762 | | |
| ABCG1 | 79 | (intron 4 198-206) | agctccccttgcctggttga TCCTCAGGG/Δ ttctacttagaatgcct | 4763 | | |
| ABCG1 | 80 | 5′untranslated region | cgcagctcaagcctcgtccc (CGC)8-10 ccccggggcatggcctgtct | 4764 | | |
| | | (−713)-(−741) | | | | |
| ABCG1 | 81 | (intron 6 376-387) | tcttgccttgagctcaagag (A)10-12 tagccaggtttctgcgcatg | 4765 | | |
| ABCG1 | 82 | (intron 7 19944-19945) | ctgatgaggaggggaggggg | 4766 | | |
| | | | (CACCAGGCAGCAGACTCTGATGAGGAGGGGAGGGGG) | | | |
| | | | caccaggcagcagactctga | | | |
| ABCG1 | 82 | (intron 7 19944-19945) | ctgatgaggaggggaggggg | 4767 | | |
| ABCG1 | 83 | (intron 7 25136-25137) | catgaacttgcctgaccata (G) ccctgtgaggagctagggct | 4768 | | |
| ABCG1 | 83 | (intron 7 25136-25137) | catgaacttgcctgaccata ccctgtgaggagctagggct | 4769 | | |
| ABCG2 | 1 | (intron 1 152) | tcatttgaaagtgggtatgc G/A gtttaaaactgacagttcaa | 4770 | | |
| ABCG2 | 2 | (intron 1 614) | agctagtcataaataaatac G/A ccagagtagtaaggaagaga | 4771 | | |
| ABCG2 | 3 | (intron 1 10002) | cctcatgaatggtatacatg T/A cccaacatatctctttcgat | 4772 | | |
| ABCG2 | 4 | (intron 1 10123) | acagtggtccctttgggtgc G/A tatacccaaatccctgcata | 4773 | | |
| ABCG2 | 5 | (intron 1 10768) | ataggaataattgagaacag G/A gtctgaagaactctgcagga | 4774 | | |
| ABCG2 | 6 | (intron 1 10791) | ctgaagaactctgcaggaaa T/C g/a aaaatagttccctgctttt | 4775 | | |
| ABCG2 | 7 | (intron 1 10792) | tgaagaactctgcaggaaa t/c G/A aaaatagttccctgctttta | 4776 | | |
| ABCG2 | 8 | (intron 1 14183) | tcacttaaggctttgcaggg T/G gtctaggacacagaaagaga | 4777 | | |
| ABCG2 | 9 | (intron 1 14934) | aaagtgtctttaaaatttcc A/G tcttgagtcagtgagctatt | 4778 | | |
| ABCG2 | 10 | (intron 1 14955) | tcttgagtcagtgagctatt G/T aaattcaagcaataagttat | 4779 | | |
| ABCG2 | 11 | (intron 1 17251) | ctgtttgggaacagcaactc A/C atcataggcagagagaaagt | 4780 | | |
| ABCG2 | 12 | (intron 1 17347) | atttcaaacctgtttcacaa G/A ttgttaagctcatcttaagg | 4781 | | |
| ABCG2 | 13 | (intron 1 17626) | gaaggtgcataacaacttcc T/G acataaagtctggagctata | 4782 | | |
| ABCG2 | 14 | (intron 1 18369) | ctattgcttttctgtctgca G/T aaagataaaaactctccaga | 4783 | | |
| ABCG2 | 15 | (coding region 34 | atgtcgaagtttttatccca G/A tgtcacaaggaaacaccaat | 4784 | | |
| | | (Val 12 Met)) | | | | |
| ABCG2 | 16 | (intron 2 36) | tgtaaaaagacagcttttta A/G tttacctacagtgaacctca | 4785 | | |
| ABCG2 | 17 | (intron 2 4230) | caaccctaaattggagggcc C/T gggcgtggtgattgagaaag | 4786 | | |
| ABCG2 | 18 | (intron 2 4518) | gttgacagacttttatagtg A/C gggacactgacctgcatgca | 4787 | | |
| ABCG2 | 19 | (intron 2 6278) | atgtatgtaccacgtcttca T/C attcttaaaggatgacccta | 4788 | | |
| ABCG2 | 20 | (intron 3 10) | ggcaaatcttcgtgagtata A/G gagagtataagtaagcgttt | 4789 | | |
| ABCG2 | 21 | (coding region 421 | tgacggtgagagaaaactta C/A agttctcagcagctcttcgg | 4790 | | |
| | | (Gln 141 Lys)) | | | | |
| ABCG2 | 22 | (intron 6 3203) | tcctattctgttttaataaa A/G gcattgaatttaggtttgct | 4791 | | |
| ABCG2 | 23 | (intron 6 3287) | gtcaggctgaactagagcaa A/G caatctaaaggcaagaatag | 4792 | | |
| ABCG2 | 24 | (intron 9 5974) | tatactaataaatggtgtgt A/T taagtttttatctctaattg | 4793 | | |
| ABCG2 | 25 | (intron 10 1908) | gacgcttatgtgcagcctat G/T ttgatgtctggaaaggctga | 4794 | | |
| ABCG2 | 26 | (intron 10 2094) | ccctgagggctgaggtatct G/A gattatttccagacttgcta | 4795 | | |
| ABCG2 | 27 | (intron 11 20) | tgtgagtaggtctttgttct A/G ggaacggggctgtccagcag | 4796 | | |
| ABCG2 | 28 | (intron 11 1447) | tgttcttcaaggaaagcccc C/T gtcaaagaaggaaaagaagc | 4797 | | |
| ABCG2 | 29 | (intron 12 49) | atgtctttagtcttgcctat G/T ggtgaagtcagttgcacctt | 4798 | | |
| ABCG2 | 30 | (intron 12 1566) | tatgcagttacatggacaga C/T acaacattggagaccgaggg | 4799 | | |
| ABCG2 | 31 | (intron 13 40) | gctctgataaggaattgttt C/T tttccttcatttcttcctgc | 4800 | | |
| ABCG2 | 32 | (intron 13 1823) | ttactcaagcaggcctgact C/T ttagtatttgctttttgtag | 4801 | | |
| ABCG2 | 33 | (intron 14 497) | ctaatgaaaacaaacaagaa T/C gaaagattgtcactgtaaat | 4802 | | |
| ABCG2 | 34 | (intron 14 815) | taactctttggaaacttctt A/G aaatttaaaactgtttacct | 4803 | | |
| ABCG2 | 35 | (intron 15 110) | ccaggggcactgaatttttc C/T gagcctacgttttctcatcc | 4804 | | |
| ABCG2 | 36 | (intron 15 566) | gccgcatagtcatgtgttgt T/A gtttttaaattaacttggaa | 4805 | | |
| ABCG2 | 37 | (intron 15 639) | aacaagaaacacttgaataa G/A ttgagaaaaaaccccgtttt | 4806 | | |
| ABCG2 | 38 | (intron 15 1197) | tgagtagctgggattacagg C/T gcccaccaccacacctggct | 4807 | | |
| ABCG2 | 39 | (5′flanking region | gttgggatggctacactcac TCAC/Δ aaagcctgatggcccgtttc | 4808 | | |
| | | (−998)-(−995) | | | | |
| ABCG2 | 40 | (intron 13 405) | ctgctagtttattttttttt T/Δ aactttttaatttatgttt | 4809 | | |
| ABCG2 | 41 | (intron 13 692-702) | tcaatatgtttctgcttatc (T)9-11 aatggttacttaatcctaat | 4810 | | |
| ABCG2 | 42 | (intron 15 645-650) | aaacacttgaataaG/Attgag (A)7-8 ccccgttttcacataatgtt | 4811 | | |
| ABCG4 | 1 | (intron 1 84) | ggcctgggtgtcccatgttc G/A gaaagtcctgcaccagtggg | 4812 | | |
| ABCG4 | 2 | (intron 2 77) | gaacacagaaggtattctga A/G agggcattgacccccatcct | 4813 | | |
| ABCG4 | 3 | (coding region 679 | tggtgtccctcatgaagtcc C/T tggcacaggggggccgtacc | 4814 | | |
| | | (Leu 227 Leu)) | | | | |
| ABCG4 | 4 | (intron 7 95) | ggcctcctaggggtagagat C/T tcaccgtcgcctgccttccc | 4815 | | |
| ABCG4 | 5 | (intron 7 158) | cttgcccttgggaagtgagt G/A tgaatctaaactgagctctc | 4816 | | |
| ABCG4 | 6 | (intron 8 106) | ccccagaggcattgcaacca A/G tgggtgctaggaagaaccta | 4817 | | |
| ABCG4 | 7 | (intron 11 1120) | acgagataagtga t/c ggtcat A/G tggccagggaggaaggggac | 4818 | | |
| ABCG4 | 8 | (intron 11 1173) | gggggacagcttgaacaaga A/G tgtggaggcaggatggacac | 4819 | | |
| ABCG4 | 9 | (3′untranslated region 2) | gagtgacaggcacatacatg A/C gaacaggccatctcagccct | 4820 | | |
| ABCG5 | 1 | (intron 3 40) | ccctggcccccccgcccgcc C/A cgggggcttaggctacactg | 4821 | | |
| ABCG5 | 2 | (intron 4 841) | gcttggaggcatcttgaatg C/T gcctcatccaaactggactg | 4822 | | |
| ABCG5 | 3 | (intron 4 1145) | gagcaaatccagcccacagc G/A tgtaaaat c/a ctgataagtaa | 4823 | | |
| ABCG5 | 4 | (intron 4 1154) | cagcccacagc g/a tgtaaaat C/A ctgataagtaattcagtggg | 4824 | | |
| ABCG5 | 5 | (intron 4 1690) | acagagatgagaaggaggct T/C gggaatctaccctggctggt | 4825 | | |
| ABCG5 | 6 | (intron 4 1806) | tcttttgttccagaatatat T/C tatatctagtttatttatgc | 4826 | | |
| ABCG5 | 7 | (intron 4 1878) | atttcagatatgtccattct C/T tgggtgggtcaaagctacat | 4827 | | |
| ABCG5 | 8 | (intron 4 2052) | gggtgtctggaaacaaaact C/T attaccatatgagtatcttc | 4828 | | |
| ABCG5 | 9 | (intron 4 2108) | tccccctggggtttctgcag A/T tagaggtaatcagtacaggg | 4829 | | |
| ABCG5 | 10 | (intron 4 2230) | agcttcttgattagaaattc G/A gtaaagaattttttttagtc | 4830 | | |
| ABCG5 | 11 | (intron 4 2318) | ggagttacaggctttaagta G/C agcgaagagaattggaagaa | 4831 | | |
| ABCG5 | 12 | (intron 4 2367) | ttaaatgtggctgggggtta C/T aaattgggtccccatttaag | 4832 | | |
| ABCG5 | 13 | (intron 4 2464) | gattatatgtctttgatgtg A/G actcacactgagattgtacc | 4833 | | |
| ABCG5 | 14 | (intron 4 2586) | aaagcatttatgataataaa G/A tttcaaaacccaaacactta | 4834 | | |
| ABCG5 | 15 | (intron 6 1318) | cagagacattcaaagtgcat C/T gctacccttgtgatcacaca | 4835 | | |
| ABCG5 | 16 | (intron 9 164) | caactattgagttaccaaca T/C gttaatatgaatgagctcac | 4836 | | |
| ABCG5 | 17 | (intron 9 365) | gtaccgttagcttctctttg A/G agctgattttaggacagcca | 4837 | | |
| ABCG5 | 18 | (intron 10 64) | tcatggagctagtgggactc G/A tgcagggagagctccagggt | 4838 | | |
| ABCG5 | 19 | (intron 10 2406) | tcaacaagcctgcttactgc G/A gttagttgtgaccattgtct | 4839 | | |
| ABCG5 | 20 | (intron 10 2442) | tgtctaagtaatttaatgtt T/G tcctatgagagctgaaggag | 4840 | | |
| ABCG5 | 21 | (intron 11 4150) | aaggccctgaaatggctgtt G/T ctggctattgttccgagctc | 4841 | | |
| ABCG5 | 22 | (intron 11 4623) | caaacagaaagaattttata C/T cttttgattgacagaaaata | 4842 | | |
| ABCG5 | 23 | (intron 11 4737) | attttcacaatgaatgttgg T/G tcggtctctccttccttttt | 4843 | | |
| ABCG5 | 24 | (intron 11 4791) | ggttagttctaactttctac G/A ttggtaccttcaactttctg | 4844 | | |
| ABCG5 | 25 | (3′untranslated region 2) | tgaggattaaaataaaaaac C/T gtaggaatgggctcaacagt | 4845 | | |
| ABCG5 | 26 | (3′flanking region 1560) | catagcactcagcaagaaac G/C tgtgctaaagactgaggttc | 4846 | | |
| ABCG5 | 27 | (intron 4 1078-1080) | gggcacagctccctgggagc AGG/Δ agaactcccgatagcagagt | 4847 | | |
| ABCG5 | 28 | (intron 10 2321-2327) | agcgggttgggtgagccctt TAACATT/Δ aggtaggtgtggtgttggc | 4848 | | |
| ABCG5 | 29 | (intron 11 422-433) | ggaattaagactagtcagac (A)10-12 gcctgcaggataaaagactg | 4849 | | |
| ABCG5 | 30 | (intron 11 3988-4004) | ctttttttgtagtctggtcc (T)15-17 cttttcctgttcttactctg | 4850 | | |
| ABCG5 | 31 | (3′untranslated region | taccctaaaacttaaagtat (A)11-13 cctaccgaaaaaaaaaaaaa | 4851 | | |
| | | 2719-2731) | | | | |
| ABCG8 | 1 | (5′untranslated region | aagagagctgcagcccaggg G/T cacagacctgtgggccccat | 4852 | | |
| ABCG8 | 2 | (intron 1 898) | cctttgactgaattcgggat A/G tggcaggatttgaagcagga | 4853 | | |
| ABCG8 | 3 | (intron 1 1548) | cctcacaacctgaaaggcca G/T gtgtaaattgagaaattcta | 4854 | | |
| ABCG8 | 4 | (intron 1 1611) | tggtacgggggagccacttc C/T agcccgagccacaacctgtc | 4855 | | |
| ABCG8 | 5 | (intron 1 3245) | tgggacaatgaagcaatgtg T/C acagtgacagcggagagggc | 4856 | | |
| ABCG8 | 6 | (intron 1 3430) | gggttgaggtgggaatggaa A/C tctggagttctactcactgg | 4857 | | |
| ABCG8 | 7 | (intron 1 3509) | tacacaaatcagcttaaaga T/A ctctcatgtacacaccacca | 4858 | | |
| ABCG8 | 8 | (intron 1 3980) | gaaaataaaccctggtcaga C/T gcttgaggtcagcctccctc | 4859 | | |
| ABCG8 | 9 | (intron 1 4123) | aagggtgttctgggctcccc G/A taagtgtttgttgggtgcat | 4860 | | |
| ABCG8 | 10 | (intron 1 5354) | cagcttctaaaggagcccct A/C atctctcctgtct t/c ccacag | 4861 | | |
| ABCG8 | 11 | (intron 1 5368) | gcccct a/c atctctcctgtct T/C ccacagggcctccaggatag | 4862 | | |
| ABCG8 | 12 | (coding region 161 | ggaggtcagagacctcaact G/A ccaggtagaggcacgcctgg | 4863 | | |
| | | (Cys 54 Tyr)) | | | | |
| ABCG8 | 13 | (intron 2 86) | gaaataaaagggtgggccca C/A cttgcaggccctctgccc c/g c | 4864 | | |
| ABCG8 | 14 | (intron 2 105) | a c/a cttgcaggccctctgccc C/G caaggacagagtccagtcca | 4865 | | |
| ABCG8 | 15 | (intron 4 43) | gacccccaggtccaagaagc C/T acagtgtccatgccccgctc | 4866 | | |
| ABCG8 | 16 | (intron 6 1035) | caggaggacaggccgcccct C/T gccctctgtactcacattct | 4867 | | |
| ABCG8 | 17 | (intron 6 1085) | cacagaaaggtcacctccct C/A cctgtgctcaggtggcagcc | 4868 | | |
| ABCG8 | 18 | (intron 6 1184) | gcacctgccgacctggccat C/T ggggaataatttaaagtaac | 4869 | | |
| ABCG8 | 19 | (coding region 1199 | tggggcggtgcagcagttta C/A gacgctgatccggtaattat | 4870 | | |
| | | (Thr 400 Lys)) | | | | |
| ABCG8 | 20 | (intron 8 137) | gaaaaaaacagcatccagca G/A ggcgttggtggcttatgcct | 4871 | | |
| ABCG8 | 21 | (intron 9 412) | ttctcttttcctttccctta T/C tttttaggttactcagagag | 4872 | | |
| ABCG8 | 22 | (intron 10 343) | aggaagcagaggttcagaga G/A gctacgtggctctccaaggc | 4873 | | |
| ABCG8 | 23 | (intron 10 614) | cttttaaacgtttataataa T/C ggcagtgaaggtgctggctt | 4874 | | |
| ABCG8 | 24 | (coding region 1695 | gcctccttcttcagcaatgc C/T ctctacaactccttctacct | 4875 | | |
| | | (Ala 565 Ala)) | | | | |
| ABCG8 | 25 | (intron 11 82) | tgctttcatctggagatgga C/T acttatcacttagatccaac | 4876 | | |
| ABCG8 | 26 | (intron 1 2882-2893) | tctcttagaaatggataaga (T)11-13 gacagagtctcacgctgtgg | 4877 | | |
| ABCG8 | 27 | (intron 1 3654) | tttatctttcccattttttt T/Δ ctgtataattttgggtcttt | 4878 | | |
| ABCG8 | 28 | (intron 1 5045) | tcagagcacagaggtttttt T/Δ atagaactctctccggtcaa | 4879 | | |
| ABCG8 | 29 | (intron 9 292-302) | tggctttactgtgcctattt (A)10-12 tgagagacttgggcaatatg | 4880 | | |
| ABCG8 | 30 | (intron 9 417-418) | tttcctttccctta t/c ttttt (T) aggttactcagagagggcaa | 4881 | | |
| ABCG8 | 30 | (intron 9 417-418) | tttcctttccctta t/c ttttt aggttactcagagagggcaa | 4882 | | |
| ABCG8 | 31 | (intron 10 28-34) | ggcagggttgagagcaagtg (C)7-9 acccaccagggtgggggtaa | 4883 | | |
| ABCG8 | 32 | (3′untranslated region 2) | tcctggggacagtgaggaca A/Δ tgaccctacagatgctcagc | 4884 | | |
| ABCE1 | 1 | (5′flanking region − 158) | aactcagattctcggcacct C/T cagcagctggcttcgccaac | 4885 | | |
| ABCE1 | 2 | (intron 9 237) | ctgaaattatatgcaaattc C/T gtagctttataggaagcaga | 4886 | | |
| ABCE1 | 3 | (intron 9 4203) | ttgtgtaggaagctgataca T/G taatttgacatatgagatgt | 4887 | | |
| ABCE1 | 4 | (intron 10 1811) | ccaagaaacttcagctttct C/T ttcacttaaatataggaaac | 4888 | | |
| ABCE1 | 5 | (intron 17 2301) | atatccagaaacagatggta T/C gtgcagaacaggttgtacag | 4889 | | |
| ABCE1 | 6 | (3′untranslated region 1 | tggatgattagactgactct G/C agaatattgataagccattt | 4890 | | |
| ABCE1 | 7 | (intron 1 5349-5363) | tttgtctgggttggttgggg (T)13-16 gagactgggtctgactctca | 4891 | | |
| ABCE1 | 8 | (intron 1 5845-5854) | tacatttgtcaaaatttata (T)9-10 gcagataatcatttcatctc | 4892 | | |
| ABCE1 | 9 | (intron 5 836-851) | taaattcacatgattctgta (T)14-16 aggatcctcctgactggcag | 4893 | | |
| ABCE1 | 10 | (intron 8 1153-1169) | tctttcaaacttatatttgc (T)13-17 catagtttcatgtttgatga | 4894 | | |
| ABCE1 | 11 | (intron 9 1023-1024) | ttgctctgtttcaaatctct (T) attcatgggccagcagctcg | 4895 | | |
| ABCE1 | 11 | (intron 9 1023-1024) | ttgctctgtttcaaatctct attcatgggccagcagctcg | 4896 | | |
| ABCE1 | 12 | (intron 9 2338-2346) | agtgtagatggacctcgggg (A)8-9 ctagttaaggaaaagtaata | 4897 | | |
| ABCE1 | 13 | (intron 9 3213-3221) | ttccaattttccattgttac (T) cttgccagattactcctgaa | 4898 | | |
| ABCE1 | 14 | (intron 10 284-299) | tcctctgcattttggcttct GCAGTATTACTGTAGT/Δ | 4899 | | |
| | | | atttgtcattttcaaattaa | | | |
| ABCE1 | 15 | (intron 10 840-853) | ttttttggtttctttctttc (T)13-14 aatcttggaggaatcttttt | 4900 | | |
| ABCE1 | 16 | (intron 16 1163-1172) | gattagaaatccaggttaaa (T)9-10 gttttgcacaaaaatattac | 4901 | | |
| ABCE1 | 17 | (intron 16 1372-1382) | taaaatttaatcaaaattga (T)10-11 ctcttagtcctcaaaccctt | 4902 | | |
| ABCF1 | 1 | (5′untranslated region | gccagccccatcggggttcc C/T cgccgccggaagcggaaata | 4903 | | |
| ABCF1 | 2 | (intron 1 101) | gcacgagactgaccgggccc C/G tgcgggagttactgcgcatg | 4904 | | |
| ABCF1 | 3 | (intron 20 69) | tgactttaaccgaccacctc C/T ctctcttctcgggcagaaaa | 4905 | | |
| ABCF1 | 4 | (intron 23 35) | agtgtgccctcatccctgct C/A catggggaccaagctgtagt | 4906 | | |
| ABCF1 | 5 | (intron 7 342-354) | acagagcgagactccgtctc (A)10-14 gaaaaaaaaaaaaaacattt | 4907 | | |
| ABCF1 | 6 | (intron 7 356-369) | cgtctcaaaaaaaaaaaaag (A)13-15 catttcatcagacctgtctt | 4908 | | |
| ABCF1 | 7 | (3′untranslated region 2) | tcagccggccccgagagtga A/Δ gctttccttcccagaagtct | 4909 | | |
| ABCF1 | 8 | (3′flanking region | attaatttgatcaattgtct (T) aatatgtcgtactctagatt | 4910 | | |
| | | 1067-1068) | | | | |
| ABCF1 | 8 | (3′flanking region | attaatttgatcaattgtct aatatgtcgtactctagatt | 4911 | | |
| | | 1067-1068) | | | | |
| GAT1 | 1 | (5′untranslated region − 1 | gcagctcggactcagctccc G/A gagcaacccagctgcggagg | 4912 | | |
| GAT1 | 2 | (5′untranslated region − 2 | gaaggcctcagcccccagcc A/G ctgggctgggcctggcccaa | 4913 | | |
| GAT1 | 3 | (intron 3 150) | caatagaacaaccttttctc G/A ggctcatgccgccctgaccc | 4914 | | |
| GAT1 | 4 | (intron 4 211) | ttctctgggcttccccactc A/C gttctccagcctgcctgctc | 4915 | | |
| GAT1 | 5 | (intron 5 33) | gagacttcccatgataacct C/T ccagggcttcacccccaaac | 4916 | | |
| GAT1 | 6 | (intron 6 168) | gaaccagatgcccccagcct C/T gactcagtcccagtctccac | 4917 | | |
| GAT1 | 7 | (intron 1 58-71) | ggaagatgggggcctttgtt (A)13-15 gtacatggagaaattaactg | 4918 | | |
| GAT1 | 8 | (intron 3 1306-1319) | aataggttgaggaggagcag (A)12-15 tcaagagtgtggagggggca | 4919 | | |
| GAT2 | 1 | (intron 4 842) | ttgacctccaaaagtgtttg G/A attacaggcatgggccattg | 4920 | | |
| GAT2 | 2 | (intron 5 183) | ccacatccatcattcgagac A/C a/c actcgtctcagctgccatg | 4921 | | |
| GAT2 | 3 | (intron 5 184) | cacatccatcattcgagac a/c A/C actcgtctcagctgccatga | 4922 | | |
| GAT2 | 4 | (coding region 1269 | actagactgctagtgtcctc C/T ggtgagcccagtcccatagg | 4923 | | |
| | | (Ser423Ser)) | | | | |
| GAT2 | 5 | (3′unstranslated region 17 | ataaatgtgtacatgagtgt A/G tgaacacaaatacataaggt | 4924 | | |
| GAT2 | 6 | (3′flanking region 1386) | tgtagcagcccacatcgcca G/A tgttcacctgagagagagag | 4925 | | |
| GAT3 | 1 | (5′flanking region − 463) | ttcctgagaggcaaatcccc T/C tcccctactcgggaggtgcc | 4926 | | |
| GAT3 | 2 | (5′untranslated region − 1) | cctgcccacagctctggctc G/A tcttgccccagtgccatgac | 4927 | | |
| GAT3 | 3 | (coding region 153 (Pro51 | cctgtccaccactgtcgccc G/A ccccacaatgcctccacagg | 4928 | | |
| GAT3 | 4 | (intron 2 177) | gcaccaagacccttggcttc T/C tcccactcagagtccaagca | 4929 | | |
| GAT3 | 5 | (intron 2 6201) | gctcatcctctctggtcctt T/G tgccccagcacaggttcctc | 4930 | | |
| GAT3 | 6 | (intron 3 79) | tctgctccacccgtgcaccc G/C caaagaggcaaagagctggg | 4931 | | |
| GAT3 | 7 | (coding region 723 | tggcgttggctgcagttaac T/A gtgtccattcccttcttcgt | 4932 | | |
| | | (Thr241Thr)) | | | | |
| GAT3 | 8 | (intron 5 524) | tcgaagtacaaaggaaagtt T/C aaagagaagcctgagcctgg | 4933 | | |
| GAT3 | 9 | (intron 7 386) | gaccaatgggtttcagactc G/A aagacaaaaattatgtttat | 4934 | | |
| GAT3 | 10 | (intron 9 81) | attgtcctgtcctctaccca G/A gggagccatcctttatgaac | 4935 | | |
| GAT3 | 11 | (5′flanking region | tacatttggtccccaggggg (G) aagcggctgatcaggagaga | 4936 | | |
| | | (−661)-(−660) | | | | |
| GAT3 | 11 | (5′flanking region | tacatttggtccccaggggg aagcggctgatcaggagaga | 4937 | | |
| | | (−661)-(−660) | | | | |
| GAT3 | 12 | (intron 8 211-212) | tctgacttggactgggcaaa AA/Δ gtatggtggtatctggatag | 4938 | | |
| GATP1 | 1 | (5′flanking region − 916) | acagagtagatgttcaataa G/A tatttgttgtatctgtgaga | 4939 | | |
| GATP1 | 2 | (5′flanking region − 843) | tagtgcagcgactatgcctt G/A atgtgtgtgtgtttgggatt | 4940 | | |
| GATP1 | 3 | (5′flanking region − 526) | aaatgtgtgcctgtatgtta T/C acatctgtacatatatttcc | 4941 | | |
| GATP1 | 4 | (5′flanking region − 172) | acaaacacaactcaaagtat G/A tgtgttattaaaagtagcta | 4942 | | |
| GATP1 | 5 | (intron 1 206) | ttgattcaggcaagttagtc C/G taaatggctttgagagactt | 4943 | | |
| GATP1 | 6 | (intron 1 454) | caacataacaataatttcct G/A taagaaaaatggccattttg | 4944 | | |
| GATP1 | 7 | (intron 1 999) | gtttagcaaggttagatatt A/G atgtggatgttaagacaaaa | 4945 | | |
| GATP1 | 8 | (intron 1 1223) | ttgctagaagctagtaggac C/T agctttataaatacagagat | 4946 | | |
| GATP1 | 9 | (intron 1 1326) | aactagttaggcaacccatg T/C gttttaggg g/a aaaagcaatg | 4947 | | |
| GATP1 | 10 | (intron 1 1336) | gcaacccatg t/c gttttaggg G/A aaaagcaatgaggtcatgat | 4948 | | |
| GATP1 | 11 | (intron 1 1498) | atagtttgctcttaagaata C/T actctgagaaggtttatagt | 4949 | | |
| GATP1 | 12 | (intron 1 5041) | ttatgctcccgaggagttag C/T tctctaaatgcataaggaga | 4950 | | |
| GATP1 | 13 | (intron 1 9532) | aaagactgggagcacttccc A/G atgacaaatactagactaga | 4951 | | |
| GATP1 | 14 | (intron 2 961) | aaaaagttatatagaaatat A/G agtgtcactcctttctagtt | 4952 | | |
| GATP1 | 15 | (intron 2 1110) | gtctactagtgttcaactcc T/C ttagatcttagcctgtatca | 4953 | | |
| GATP1 | 16 | (intron 2 1419) | aaagcctaagaaggatgcag T/C gcaatagcctatgtgagaag | 4954 | | |
| GATP1 | 17 | (intron 2 3339) | tatggtttgcaaaaaactta T/C cctctctaggagcaatgctt | 4955 | | |
| GATP1 | 18 | (intron 3 66) | caggaaatgaagttgcactt T/C cctctctaggagcaatgctt | 4956 | | |
| GATP1 | 19 | (intron 3 205) | tcagttttgtcaatttacac A/G atggggatttgggacctttt | 4957 | | |
| GATP1 | 20 | (intron 3 6377) | aatgaatagactttgagtta C/T tggatttttagtggataaat | 4958 | | |
| GATP1 | 21 | (intron 3 7238) | tgaatgtcacattttttaaa G/A tttgtgttccttatctcata | 4959 | | |
| GATP1 | 22 | (intron 4 1016) | ttttattctggattcatgtt T/C gtggaaattgcagtagtcca | 4960 | | |
| GATP1 | 23 | (intron 5 110) | tccacaatgatgagtagagt A/G tcttggcacagttggccttc | 4961 | | |
| GATP1 | 24 | (intron 6 496) | agtgtctgaattataagcca A/G ttttatagttggttgggacc | 4962 | | |
| GATP1 | 25 | (intron 7 1934) | aaagtgaaaggaaattaaaa G/C tgagaacttgagcctgaatg | 4963 | | |
| GATP1 | 26 | (intron 7 2140) | tagaatgtaccaaatgaatc A/G gcatctctgaggatgggacc | 4964 | | |
| GATP1 | 27 | (intron 7 2365) | tgaaatcttctttatcaact C/T gattttcctccagactttac | 4965 | | |
| GATP1 | 28 | (intron 8 88) | gcaaactcctaagttgaagt G/C ttttaggatattttttgact | 4966 | | |
| GATP1 | 29 | (intron 9 534) | tcatattttgtattttaaag G/A ttatctgggttttactgaaa | 4967 | | |
| GATP1 | 30 | (intron 9 1286) | tattcttctgagataaatca T/C tgaaggagtggctatgtggt | 4968 | | |
| GATP1 | 31 | (intron 11 215) | ttcactcctattcctcgcta C/T ttttcttccttatttcttag | 4969 | | |
| GATP1 | 32 | (intron 11 663) | ttcttcttcttttggagctc T/A aaagtagagttcagttaatc | 4970 | | |
| GATP1 | 33 | (intron 11 999) | atcatcactgcatgagagtt A/G gaattatctaactttgtgat | 4971 | | |
| GATP1 | 34 | (intron 11 16727) | tttcttttatttacaaactt A/G tttacttttcaggtgtatga | 4972 | | |
| GATP1 | 35 | (intron 12 48) | ctatcagaacaatattatta T/G tattattttttattacactt | 4973 | | |
| GATP1 | 36 | (intron 12 686) | tatgttttgataaactttgc C/A gtacaaataaagaaaattga | 4974 | | |
| GATP1 | 37 | (intron 12 708) | tacaaataaagaaaattgaa A/G tatttccaaataaatcaagt | 4975 | | |
| GATP1 | 38 | (intron 13 418) | tctctggtctccaaaatcat A/G tattttctccctcttta c/a at | 4976 | | |
| GATP1 | 39 | (intron 13 436) | at a/g tattttctccctcttta C/A attttgctgaaacaatcttc | 4977 | | |
| GATP1 | 40 | (3′untranslated region 2) | gtctttaagaacctaaaaaa C/A ctcttaactcaaaataataa | 4978 | | |
| GATP1 | 41 | (3′flanking region 57) | agtgactaaagtttttctta C/A aaacaagtgtctgaatcaaa | 4979 | | |
| GAPT1 | 42 | (3′flanking region 572) | aatacactatggttatttat G/A tgtactataaatggagtgag | 4980 | | |
| GATP1 | 43 | (3′flanking region 788) | atttcctaaatgatcagatg C/T atcatatgaaaaaagaaagc | 4981 | | |
| GATP1 | 44 | (3′flanking region 1356) | aggtgactgacataaatggg G/A gcagaggacataatgaggtt | 4982 | | |
| GATP1 | 45 | (5′untransalted region | attttctaatctgtattaaa (A) gcgttccaggtatttttgta | 4983 | | |
| | | (−189)-(−188) | | | | |
| GATP1 | 45 | (5′untranslated region | attttctaatctgtattaaa gcgttccaggtatttttgta | 4984 | | |
| | | (−189)-(−188) | | | | |
| GATP1 | 46 | (intron 4 725-726) | tgatctttaatagcggggaa AA/Δ caggcaagtacgctatagtt | 4985 | | |
| GATP1 | 47 | (intron 4 1082-1083) | attgagtcaggaaaccaaaa CA/Δ gtttcaaaaatttgaaaaat | 4986 | | |
| GATP1 | 48 | (intron 4 2301) | aatgtcatgtcttttttttt T/Δ aatgcagagtgtacaaagga | 4987 | | |
| GATP1 | 49 | (intron 9 241-46) | attgtatgtgcatgtgggtg TGTGTG/Δ catgattgtctttgtgatat | 4988 | | |
| GATP2 | 1 | (5′flanking region − 2574) | ggataaggcaacccctatgt A/G tcactgctgcaggagaggga | 4989 | | |
| GATP2 | 2 | (5′flanking region − 1723) | tctttcagacttcaaaggcc A/G tgatatttcatcagagctgt | 4990 | | |
| GATP2 | 3 | (5′flanking region − 1180) | tgcttatttaacaggcataa T/G ctttggtctcctgagccaga | 4991 | | |
| GATP2 | 4 | (5′flanking region − 811) | tatgtgcatatgtgtataca G/A gtaaaagtgtgtatatatgt | 4992 | | |
| GATP2 | 5 | (intron 1 7188) | aatcatttgaaatttaagaa A/G aaaatatgttcagagaaaaa | 4993 | | |
| GATP2 | 6 | (intron 1 7331) | gtgaaatgaggaacaaagtg T/C ccacctttttttcctgaata | 4994 | | |
| GATP2 | 7 | (intron 1 7391) | agagagatgtgaaatagtat T/G tttctggggaagtaggggaa | 4995 | | |
| GATP2 | 8 | (intron 1 7886) | ttgttagtagaaagaaaatc G/A aagcctaaaactaaaggaag | 4996 | | |
| GATP2 | 9 | (intron 1 7958) | ttgctattatataatttttt T/A a/t aaaaaagatttcctaatat | 4997 | | |
| GATP2 | 10 | (intron 1 7959) | tgctattatataattttttt t/a A/T aaaaaagatttcctaatat | 4998 | | |
| GATP2 | 11 | (intron 1 8036) | ggaaaaaatggggtgaaatt A/T atcaaagggcagcttattac | 4999 | | |
| GATP2 | 12 | (intron 1 9164) | acattatattctatataaaa G/T agtcagttgaagtaaaaagt | 5000 | | |
| GATP2 | 13 | (intron 2 193) | tgattaagtatttctttggc G/A aaatttttgatgcttaatag | 5001 | | |
| GATP2 | 14 | (intron 2 1020) | ttgagtaacatttaggccaa G/A tggcagtcataaggaaaaag | 5002 | | |
| GATP2 | 15 | (intron 2 14865) | agaggaattaatcataagag G/T tttatttggctaaagtgaca | 5003 | | |
| GATP2 | 16 | (intron 2 14931) | gttagttaataacagaaaaa A/T tatcagaaattttaaaaaaa | 5004 | | |
| GATP2 | 17 | (intron 2 15417) | ttctaaaataagtaagctaa A/T tattctatattatactacta | 5005 | | |
| GATP2 | 18 | (intron 2 20823) | ttgtataagagatacaaaac A/C aattcctactaggggaaata | 5006 | | |
| GATP2 | 19 | (intron 2 20852) | ctaggggaaataaagcttca G/C taaggaggtggcattaagct | 5007 | | |
| GATP2 | 20 | (intron 2 21360) | ttcaaaagctgtatttctca T/C tagtgctttttgtgaataaa | 5008 | | |
| GATP2 | 21 | (intron 2 21467) | tatatacacaatacctgtcc A/G gaagatgtggtataagccaa | 5009 | | |
| GATP2 | 22 | (intron 2 21621) | tatcaatacttatgaagaga A/G ctaactattctaactaggga | 5010 | | |
| GATP2 | 23 | (intron 2 22760) | ttccccacctcctgttggtt C/G tcctcttaaacttctccttg | 5011 | | |
| GATP2 | 24 | (intron 2 23199) | cctatctgcacataacatta C/T aaacttatggcaattata a/g a | 5012 | | |
| GATP2 | 25 | (intron 2 23218) | a c/t aaacttatggcaattata A/G aactcaatacatattatact | 5013 | | |
| GATP2 | 26 | (intron 2 23330) | gcccttgttcctgttcctct G/A tacctgcctcaactacatag | 5014 | | |
| GATP2 | 27 | (intron 2 23673) | ctggagacggtagctcaaac T/C gaggatgaaaatagacattt | 5015 | | |
| GATP2 | 28 | (intron 3 89) | ggttatcaactggggtaaat T/G tatctctcacaggcaatttg | 5016 | | |
| GATP2 | 29 | (intron 3 224) | tgctaaatattctataatgc A/G caaagaatgatgtaactgaa | 5017 | | |
| GATP2 | 30 | (intron 4 97) | ccctttaaataggcagttac C/A ttttgagaagatacccacta | 5018 | | |
| GATP2 | 31 | (intron 4 568) | ttcatgatccaaattgtggc A/G acgtatttccaggcaacaag | 5019 | | |
| GATP2 | 32 | (intron 4 599) | aggcaacaagatagaagaag A/G aaagaataagaagcaacaaa | 5020 | | |
| GATP2 | 33 | (intron 4 753) | aaaatagacattattccaag T/A taccaagttcccggttaaaa | 5021 | | |
| GATP2 | 34 | (intron 4 781) | ttcccggttaaaaatcccaa G/C tataattactgtggaaggaa | 5022 | | |
| GATP2 | 35 | (intron 4 1196) | aaggaccacaatctagatca G/T cattgctctaaatatgccat | 5023 | | |
| GATP2 | 36 | (intron 4 1229) | tatgccataatatgtgacac T/C tttgcacctggtatttctac | 5024 | | |
| GATP2 | 37 | (intron 4 1623) | catctagttgaaatggatta G/C atttttattttactacattt | 5025 | | |
| GATP2 | 38 | (coding region 388 | attctaaagaaactaatatc A/G attcatcagaaaattcaaca | 5026 | | |
| | | (Asn130Asp)) | | | | |
| GATP2 | 39 | (coding region 452 | taatcaaattttatcactca A/G tagagcatcacctgagatag | 5027 | | |
| | | (Asn151Ser)) | | | | |
| GATP2 | 40 | (intron 5 165) | ttaatatacacagttcgccc A/T ttaacaacacaggtttaaac | 5028 | | |
| GATP2 | 41 | (intron 5 189) | acaacacaggtttaaactac G/A c g/a ttttcacttctatgcaaa | 5029 | | |
| GATP2 | 42 | (intron 5 191) | aacacaggtttaaactac g/a G/A ttttcacttctatgcaaatt | 5030 | | |
| GATP2 | 43 | (intron 5 507) | atataactttgctttcattg C/T aaaaggcaaact a/g ttatatc | 5031 | | |
| GATP2 | 44 | (intron 5 520) | ttcattg c/t aaaaggcaaact A/G ttatatcatttaaagacttt | 5032 | | |
| GATP2 | 45 | (intron 5 856) | agtcatgataaacctaatag A/G ataaaacaacaaaaaagaaa | 5033 | | |
| GATP2 | 46 | (intron 5 1157) | acagataatttttacttgtt T/C gtgcttttctgtatgatatg | 5034 | | |
| GATP2 | 47 | (intron 5 1226) | ccttgattgtaataatctcc A/C c a/c tgccaagagtggggccag | 5035 | | |
| GATP2 | 48 | (intron 5 1228) | ttgattgtaataatctcc c/a c A/C tgccaagagtggggccaggt | 5036 | | |
| GATP2 | 49 | (intron 5 1384) | actgttctcgtggtaatgaa G/T aagtctcacaagatctgatg | 5037 | | |
| GATP2 | 50 | (intron 5 1348) | ttataaatgagagttcccct G/A caaaagctctcttgcctgcc | 5038 | | |
| GATP2 | 51 | (intron 5 1407) | ttgctcttccttcatcttcc G/A ccatgattgtgaggcccccc | 5039 | | |
| GATP2 | 52 | (coding region 521 | gtcatacatgtggatatatg T/C gttcatgggtaatatgcttc | 5040 | | |
| | | (Val174Ala)) | | | | |
| GATP2 | 53 | (coding region 571 | gggagactcccatagtacca T/C tggggctttcttacattgat | 5041 | | |
| | | (Leu191Leu)) | | | | |
| GATP2 | 54 | (coding region 597 | ctttcttacattgatgattt C/T gctaaagaaggacattcttc | 5042 | | |
| | | (Phe199Phe)) | | | | |
| GATP2 | 55 | (intron 7 33) | agaacaaggtaccatgataa C/T gtctttctaagcacacatgc | 5043 | | |
| GATP2 | 56 | (intron 7 267) | caaaataaccaaatgtaaaa T/A gtctccctcccaaactgact | 5044 | | |
| GATP2 | 57 | (intron 7 1260) | gtaatctcacatttctctgc A/G tttacacttggtaaaacttt | 5045 | | |
| GATP2 | 58 | (intron 7 2273) | ttctcacgtcctatctagcg C/T gattatgacccttagttact | 5046 | | |
| GATP2 | 59 | (intron 8 207) | gtggaagagaattaggtttg T/C actttttagcagggagaaac | 5047 | | |
| GATP2 | 60 | (intron 8 546) | tcgggagaagtttctcccta T/C gtaattagagtaatattt a/t t | 5048 | | |
| GATP2 | 61 | (intron 8 565) | a t/c gtaattagagtaatattt A/C ttttggtaattatctatcta | 5049 | | |
| GATP2 | 62 | (intron 8 668) | taagtaatgtaaattaggat G/T catcagcatttgacagtgcc | 5050 | | |
| GATP2 | 63 | (intron 8 739) | tggagaaccattgagagtca A/G taaacaaagagaatgacttg | 5051 | | |
| GATP2 | 64 | (intron 9 112) | atttttagtaatacaggataa G/C tataattttcttgtatctt | 5052 | | |
| GATP2 | 65 | (intron 9 266) | ttagaggtagtatctgtata A/G ttggatcttataatttagtg | 5053 | | |
| GATP2 | 66 | (intron 9 305) | tgctaagatctgagacaaac C/G cttttgtaattataatcatt | 5054 | | |
| GATP2 | 67 | (intron 11 10224) | tacacttgttccataaaaaa T/C tcctctatattattcctagt | 5055 | | |
| GATP2 | 68 | (intron 11 10359) | attaatagattcaacgtgag G/C ttcccttaaactttagccta | 5056 | | |
| GATP2 | 69 | (intron 11 10916) | cttatatagaaagaaatcca C/G aaaactattttaccttttat | 5057 | | |
| GATP2 | 70 | (intron 11 10997) | aatatattagtttgaacaag T/C gagacttcactaaatataat | 5058 | | |
| GATP2 | 71 | (intron 11 11018) | gagacttcactaaatataat G/A caatgtatttgcagcactgt | 5059 | | |
| GATP2 | 72 | (intron 12 442) | aacattccaaaacttttaat C/T ga c/t t c/a acagcatgacttt | 5060 | | |
| GATP2 | 73 | (intron 12 445) | attccaaaacttttaat c/t ga C/T t c/a acagcatgacttttat | 5061 | | |
| GATP2 | 74 | (intron 12 447) | tccaaaacttttaat c/t ga c/t t C/A acagcatgacttttataa | 5062 | | |
| GATP2 | 75 | (intron 12 907) | aatgaaaagaagctggcaga T/C tgaaacatactgaatgagag | 5063 | | |
| GATP2 | 76 | (intron 113 65) | tatatatatatatatatata C/T acacacacatacatatatta | 5064 | | |
| GATP2 | 77 | (intron 13 870) | aattctgagtatcctatttc G/A atgtatccaatctgtggcac | 5065 | | |
| GATP2 | 78 | (intron 13 1935) | taaaaaaaaaaaaagtctgc T/C tttacagcaattgagccaag | 5066 | | |
| GATP2 | 79 | (intron 13 2261) | aacgaatcctccaaattttt G/C aacttttatttaatcaaaat | 5067 | | |
| GATP2 | 80 | (intron 14 248) | tcaaggataataaccaactt G/A tcaaaaatcagagataatag | 5068 | | |
| GATP2 | 81 | (intron 14 2463) | atttgtttactaatatggaa C/G cttcttcaagacatattttt | 5069 | | |
| GATP2 | 82 | (intron 14 2857) | tcatcatgtatttccaggac A/T cctggcaagatgctcctcag | 5070 | | |
| GATP2 | 83 | (intron 14 11458) | atctccagaggtcctgctgt C/T tccccaaagtccactgaccc | 5071 | | |
| GATP2 | 84 | (3′untranslated region 22) | ataataaaacaaactgtagg T/C agaaaaaatgagagtactca | 5072 | | |
| GATP2 | 85 | (3′untranslated region 24) | tcttaataaaacaaatgagt A/G tcatacaggtagaggttaaa | 5073 | | |
| GATP2 | 86 | (3′untranslated region 25) | cagagtttgaactataatac T/G aaggcctgaagtctagcttg | 5074 | | |
| GATP2 | 87 | (3′untranslated region 25) | gcctgaagtctagcttggat A/G tatgctacaataatatctgt | 5075 | | |
| GATP2 | 88 | (intron 1 457-458) | taattggcaaacataaaaaa (A) caggtgtctcaaagtcacat | 5076 | | |
| GATP2 | 88 | (intron 1 457-458) | taattggcaaacataaaaaa caggtgtctcaaagtcacat | 5077 | | |
| GATP2 | 89 | (intron 1 753-7538) | gatcagcattacaaccaaga (G) atggagaatgacattcagga | 5078 | | |
| GATP2 | 89 | (intron 1 753-7538) | gatcagcattacaaccaaga atggagaatgacattcagga | 5079 | | |
| GATP2 | 90 | (intron 1 10032-10035) | tgtgtgattctatattactt ACTT/Δ gtttcaaatttctctccaca | 5080 | | |
| GATP2 | 91 | (intron 1 10058-10061) | ttcaaatttctctccacaaa TTTA/Δ tttttctattaaattgtaat | 5081 | | |
| GATP2 | 92 | (intron 2 413-423) | acttatttaaaaattctttt (A)11-13 caaaaacaggattttaaaaa | 5082 | | |
| GATP2 | 93 | (intron 3 1595-1603) | ttgccaagtaattcaagtgc (T)8-10 gtatttaaaacaacttttca | 5083 | | |
| GATP2 | 94 | (intron 4 10-23) | ttcatgggatagtaagtgtt (A)12-14 cctctgtgccactatcagta | 5084 | | |
| GATP2 | 95 | (intron 5 1567-1572) | gtgaatataaattacttgta CTTGTA/Δ atttcaagcagatgcaactg | 5085 | | |
| GATP2 | 96 | (intron 5 1577-1585) | attacttgtacttgtaaatt (A)9-10 taagtagaataattaagagt | 5086 | | |
| GATP2 | 97 | (intron 8 1939-1941) | ttctctaactccttctactc CTT/Δ atttcaagcagatgcaactg | 5087 | | |
| GATP2 | 98 | (intron 10 3077-3078) | aaattctttatctacttttt (CTT) ttccctctttctctgctttc | 5088 | | |
| GATP2 | 98 | (intron 10 3077-3078) | aaattctttatctacttttt ttccctctttctctgctttc | 5089 | | |
| GATP2 | 99 | (intron 11 11011) | aacaag t/c gagacttcactaa A/Δ tataat g/a caatgtattt | 5090 | | |
| GATP2 | 100 | (intron 12 1160-1169) | agcatgacatggtagagatg (A)9-11 gcatttttaacatttgttaa | 5091 | | |
| GATP2 | 101 | (intron 12 1310-1312) | tccatcttaatataaaatgt TGT/Δ ctactcaaaaggagaagtct | 5092 | | |
| GATP2 | 102 | (intron 13 9-34) | tacgagcactaggtatgatg (A)24-27 tatatatatatatatatata | 5093 | | |
| GATP2 | 103 | (intron 13 35-64) | aaaaaaaaaaaaaaaaaaaa (TA)10-21 c/t acacacacatacatat | 5094 | | |
| GATP2 | 104 | (intron 13 1379-1387) | aaaattattcaccacaatac (A)8-10 caaagtaaagttatgaacac | 5095 | | |
| GATP2 | 105 | (intron 13 1916-1928) | aattctcttaaaataatgtt (A)11-13 gtctgc t/c tttacagcaa | 5096 | | |
| GATP2 | 106 | (intron 14 588-596) | caattatactttacctcttt (A)8-10 ctaatttcaaattcatatat | 5097 | | |
| GATP8 | 1 | (5′flanking region − 1413) | aataggggcttaataactct G/C aaacttatgatttctcatat | 5098 | | |
| GATP8 | 2 | (intron 1 38962) | atgaaattagtttaaaaata G/A caaccttaactatactcctc | 5099 | | |
| GATP8 | 3 | (intron 2 253) | acagacttaccaacaaagaa T/G tatccttcccaaaatgtcta | 5100 | | |
| GATP8 | 4 | (intron 2 329) | actcatggtttgcaaattaa C/G tttttaggaaactttatctc | 5101 | | |
| GATP8 | 5 | (intron 2 2568) | ccattctggtgctttctttc G/A tgaaactattttccatcagt | 5102 | | |
| GATP8 | 6 | (intron 2 2679) | ctcttattgctcttcttcca T/C gttttaatctaaataattta | 5103 | | |
| GATP8 | 7 | (intron 2 2753) | caggaaactttcacaaagcc C/A ctaattaatttaagctccct | 5104 | | |
| GATP8 | 8 | (intron 2 3132) | tggtttaatgtaggagagtt T/C accttcacagttaaattaca | 5105 | | |
| GATP8 | 9 | (intron 2 3193) | aatgtcttgggcatatttgc A/G ttcatttggggca t/c tcagtt | 5106 | | |
| GATP8 | 10 | (intron 2 3207) | atttgc a/g ttcatttggggca T/C tcagttctactagatacaaa | 5107 | | |
| GATP8 | 11 | (coding region 334 | gaactggaagtattttgaca T/G ctttaccacatttcttcatg | 5108 | | |
| | | (Ser112Ala)) | | | | |
| GATP8 | 12 | (intron 3 76) | agaattttatttttatactt G/A taagtgggcagttacctttt | 5109 | | |
| GATP8 | 13 | (intron 3 2443) | tcaatttcatgttgctctta C/T agttataggtattctaaaga | 5110 | | |
| GATP8 | 14 | (intron 4 67) | taatcacgtctataaagttt C/G tgatattctttaacaaaatt | 5111 | | |
| GATP8 | 15 | (intron 4 91) | tattctttaacaaaattgat T/A taagaacaaataggaagaac | 5112 | | |
| GATP8 | 16 | (intron 4 197) | ggtttgaactgcacctgttc G/A cttatatgcagcttttgtcc | 5113 | | |
| GATP8 | 17 | (intron 4 813) | tttaacagaataaaaaaaaa T/A attttgtaacgacaaaagaa | 5114 | | |
| GATP8 | 18 | (intron 4 974) | atatgcaccttaaaaataac C/G tggatttttaaatatgtaat | 5115 | | |
| GATP8 | 19 | (intron 4 1003) | taaatatgtaatgtacataa G/T gaatattatgcatattttgt | 5116 | | |
| GATP8 | 20 | (intron 6 155) | cattaataatcagaataaaa A/G agaaatttagctcctattta | 5117 | | |
| GATP8 | 21 | (intron 6 750) | atccaactggggtttagatt T/G cctctttctgcctctcctcc | 5118 | | |
| GATP8 | 22 | (intron 6 780) | gcctctcctccatctgcacc C/T tctcttttcctcagcaaaca | 5119 | | |
| GATP8 | 23 | (intron 6 1248) | ctatgccctgtaatctcaca C/T ttccctttatttaaaattgg | 5120 | | |
| GATP8 | 24 | (intron 6 1500) | tcgtgtctgtgttagcatat A/G ataactcatcagggtttgtg | 5121 | | |
| GATP8 | 25 | (intron 6 2008) | ataacataaatgagtaaaga A/G tatcaagggcaggaaattag | 5122 | | |
| GATP8 | 26 | (intron 6 2087) | actactctccccatacacac T/C aaaactcatgtgctccccag | 5123 | | |
| GATP8 | 27 | (intron 6 12305) | tcatctatggaggactgcaa T/C cattatcattatttcccaga | 5124 | | |
| GATP8 | 28 | (intron 7 363) | taacaaatgataccagccat C/G atactattctctggtaatag | 5125 | | |
| GATP8 | 29 | (intron 7 411) | cctttattttttgagaacct G/A gtggatgatattaaga c/a gta | 5126 | | |
| GATP8 | 30 | (intron 7 428) | cct g/a gtggatgatattaaga C/A gtatatagatcactgtaata | 5127 | | |
| GATP8 | 31 | (intron 7 634) | aaaattatatatatacatat A/G taatcttacctaagtattca | 5128 | | |
| GATP8 | 32 | (intron 7 1791) | tgtttttttaagggtagtga T/C gtgaatagtaaagcgaattt | 5129 | | |
| GATP8 | 33 | (intron 7 2000) | agttgagcaaattgctctca G/A gtagcataatgtcacttgaa | 5130 | | |
| GATP8 | 34 | (intron 7 2043) | gtttattgatccatttttta A/G tggatcaacattgtagtgag | 5131 | | |
| GATP8 | 35 | (intron 7 2171) | atttattttgagcaaaggtc G/A c g/a actct c/t cttagaaagc | 5132 | | |
| GATP8 | 36 | (intron 7 2173) | ttattttgagcaaaaggtc g/a c G/A actct c/t ttagaaagcct | 5133 | | |
| GATP8 | 37 | (intron 7 2179) | tgagcaaaggtc g/a c g/a actct C/T ttagaaagcctcacaaat | 5134 | | |
| GATP8 | 38 | (intron 7 2219) | atttctccatcttgt a/g ata T/A catcgttgttctgcatttga | 5135 | | |
| GATP8 | 39 | (intron 7 2261) | cagatattaatatatatttt A/T ttattgaaatatgttatttt | 5136 | | |
| GATP8 | 40 | (intron 8 150) | acaaaatttctccatcttgt A/G ata t/a catcgttgttctgcat | 5137 | | |
| GATP8 | 41 | (intron 8 154) | aatttctccatcttgt a/g ata T/A catcgttgttctgcatttga | 5138 | | |
| GATP8 | 42 | (intron 8 1303) | ttttttttgagatggagtct C/T gctctgttgcccaggctggg | 5139 | | |
| GATP8 | 43 | (intron 8 1372) | aagctccgcctcccaggttc T/G ccacccttctcttaaagaaa | 5140 | | |
| GATP8 | 44 | (coding region 1272) | tccttcttgtttcaacttct A/G tatttccctctaatctgcga | 5141 | | |
| | | (Leu24Leu)) | | | | |
| GATP8 | 45 | (intron 10 63) | tcacagatttgatttaataa A/T tacttatcaaatcttcctat | 5142 | | |
| GATP8 | 46 | (intron 10 911) | cttgcccaatatcctaccaa C/T gtattattaaacggcatgga | 5143 | | |
| GATP8 | 47 | (intron 10 972) | tcctagtttccttgaagata G/A gctacaactttagtaaactt | 5144 | | |
| GATP8 | 48 | (intron 10 1101) | tccctggtcctgtgttgtcc A/T g t/c agtgaagacctgaaagag | 5145 | | |
| GATP8 | 49 | (intron 10 1103) | cctggtcctgtgttgtcc a/t g T/C agtgaagacctgaaagagag | 5146 | | |
| GATP8 | 50 | (intron 10 2027) | cccattttcatgagtggcta A/G g/a ttttgtcccgtttcaaaact | 5147 | | |
| GATP8 | 51 | (intron 10 2028) | ccattttcatgagtggctaa/g G/A ttttgtcccgtttcaaacta | 5148 | | |
| GATP8 | 52 | (intron 10 2372) | tgtatttggtaaatgtattt G/T ttaatatttcaaaaactatt | 5149 | | |
| GATP8 | 53 | (intron 11 10538) | caacagaggatcaatgtaaa T/G gaaatctcttaaattaaaca | 5150 | | |
| GATP8 | 54 | (intron 12 55) | ataaatattaatgttaaata C/T taaagactgaatgcaattaa | 5151 | | |
| GATP8 | 55 | (intron 12 1802) | taaaatgaatcggtaaaaca T/G tcatgtataaatcactgtca | 5152 | | |
| GATP8 | 56 | (intron 12 2612) | ataggcatataatactcttt C/A ttccctctgtatatagggag | 5153 | | |
| GATP8 | 57 | (coding region 1833 | aacagctgtggagcacaagg G/A gcttgtaggatatataattc | 5154 | | |
| | | (Gly611Gly)) | | | | |
| GATP8 | 58 | (5′flanking region | tacataacatatacctatat CTAT/Δ gttatgtgtctgcttatata | 5155 | | |
| | | (−1590)-(−1587) | | | | |
| GATP8 | 59 | (5′untranslated region | agcatcagcaacaattaaaa ATATTCACTTGGTATCTG/Δ | 5156 | | |
| | | (−28)-(−11) | tagtttaataatggaccaac | | | |
| GATP8 | 60 | (5′untranslated region | tattcacttggtatctgtag TTTA/Δ ataatggaccaacatcaaca | 5157 | | |
| | | (−7)-(−4) | | | | |
| GATP8 | 61 | (intron 4 213-214) | ttc g/a cttatatgcagctttt (T) gtccaaccaaacagaaggag | 5158 | | |
| GATP8 | 61 | (intron 4 213-214) | ttc g/a cttatatgcagctttt gtccaaccaaacagaaggag | 5159 | | |
| GATP8 | 62 | (intron 4 505) | tataactttctctttataaa G/Δ atgcaaaatgttatagcatt | 5160 | | |
| GATP8 | 63 | (intron 4 616) | aatgaagtggaggaaaaaaa A/Δ tgatttcaagttttctgtct | 5161 | | |
| GATP8 | 64 | (intron 4 804-812) | acatccatgtttaacagaat (A)9-11 t/a attttgtaacgacaaaag | 5162 | | |
| GATP8 | 65 | (intron 4 855) | agattgtttaaccaaattag G/Δ aaactattattcaacacact | 5163 | | |
| GATP8 | 66 | (intron 7 619-628) | ttttatatatgaattaaaat (AT)4-5 catat a/g taatcttaccta | 5164 | | |
| GATP8 | 67 | (intron 7 1773-1779) | attttctatattatgaactg (T)7-8 aagggtagtga t/c gtgaata | 5165 | | |
| GATP8 | 68 | (intron 8 1270-1290) | tagtgtgccacccttctctc (T)19-23 gagatggagtct c/t gctc | 5166 | | |
| GATP8 | 69 | (intron 10 665) | aactcaaaggcttttttttt T/Δ ccatgtgacacatatcctgt | 5167 | | |
| GATP8 | 70 | (intron 11 247-250) | aaaaatcttaaggcacacac TGAT/Δ tgacagttgccttgattgta | 5168 | | |
| GATP8 | 71 | (intron 12 1622-1630) | aaataaattgttggcatcta (T)8-10 atttttctaagggtcgctgt | 5169 | | |
| GATP8 | 72 | (3′untranslated region | cctgatgcctttaaaaaaaa A/Δ tgaaacactttggatgtatt | 5170 | | |
| | | 2464-2465) | | | | |
| TAP1 | 1 | 5′flanking − 673 | agctaagagtcaaagcaccc G/C ctttttccaccagcctcgcg | 5171 | | |
| TAP1 | 2 | 5′flanking − 646 | ccaccagcctcgcgtgcctg T/G tcccttcacggacactctag | 5172 | | |
| TAP1 | 3 | 5′flanking − 563 | ttgcaagcgctggctgctac A/C ggcgacctccctgcgctccc | 5173 | | |
| TAP1 | 4 | 5′flanking − 236 | gctttgcgcgcggcgctaac G/T tgtgtagggcagatctgccc | 5174 | | |
| TAP1 | 5 | intron 3 + 408 | aaggaaactgaggccaagac C/T ctaaatgctgaaactgcaca | 5175 | | |
| TAP1 | 6 | exon 4 + 153 | ccctcaccatggtcaccctg A/G tcaccctgcctctgcttttc | 5176 | | |
| TAP1 | 7 | intron 4 + 289 | gtatttctttagcatccaag G/T ggcatagctgtgtctctttc | 5177 | | |
| TAP1 | 8 | intron 4 + 291 | atttctttagcatccaaggg C/G catagctgtgtctctttctc | 5178 | | |
| TAP1 | 9 | intron 5 + 1139 | ttccttcaggttaatgactg C/T ggttctttgtgtcccctcca | 5179 | | |
| TAP1 | 10 | intron 7 + 375 | gtctctgcccttgtctttgc C/T gcttcttctatctctactcc | 5180 | | |
| TAP1 | 11 | 3′flanking + 71 | agcgcacttttcagctgcgg G/A tgtctcctcttttatcatcc | 5181 | | |
| TAP1 | 12 | 3′flanking + 129 | aactgcatcaccttttccct T/C aagctttttaattcctatga | 5182 | | |
| TAP1 | 13 | 3′flanking + 459 | cattcagggaggcccaggtc G/A tgtgacgtcgacagttgctg | 5183 | | |
| TAP2 | 1 | intron 3 + 8 | tctcctttggcaggtaggtg G/A tgggcagctgggtccatttg | 5184 | | |
| TAP2 | 2 | intron 4 + 104 | cttcacccgtatgccaggac C/T tggggatgcttttctcttgt | 5185 | | |
| TAP2 | 3 | intron 10 + 219 | gcagcagtggtgctccctcc A/G tgggcagccccgtcaggtcc | 5186 | | |
| TAP2 | 4 | intron 11 + (317-319) | atggtgcccaggtggatgtg GTG/Δ tccatctcattcctgtcttt | 5187 | | |
| TAP2 | 5 | exon 12 + 19 | agctgcaggactggaattcc T/C gtggggatcgcacagtgctg | 5188 | | |
| TAP2 | 6 | exon 12 + (356-357) | aggtggggtggggtggggtg GG/TGGTGGGGTGGA ggctgtctgtgtcc | 5189 | | |
| OCTN1 | 1 | intron1+ 6602 | aggcgagccaggttatgtgg C/T gaaggataaggcctcttccc | 5190 | | |
| OCTN1 | 2 | intron1+ 6790 | gacaaaaggggaaaaccttc C/T gtgataggcaggtttgtgga | 5191 | | |
| OCTN1 | 3 | intron1+ 14019 | cactgtctcccactgggccc G/A ccatgtcactgttaaccaca | 5192 | | |
| OCTN1 | 4 | intron1+ 14136 | ccggtttcctaagaaaagcc T/C tttctaaaggacccctctta | 5193 | | |
| OCTN1 | 5 | intron1+ 14266 | agctttccaaaaagacactt G/T cggcaccataactccccaaa | 5194 | | |
| OCTN1 | 6 | intron1+ 14412 | cttggggcaaacggccactg C/T gtgtgcatggctcttcctgt | 5195 | | |
| OCTN1 | 7 | intron1+ 15776 | acataggagacacttctttc G/A gatctcagtattcagaacaa | 5196 | | |
| OCTN1 | 8 | intron1+ 15817 | ctgtgcttctgcgaataagc A/G gactacttcggatactgtaa | 5197 | | |
| OCTN1 | 9 | intron1+ 15889 | agagccagttttggagcccc G/A tctggcaagcaggcaggccc | 5198 | | |
| OCTN1 | 10 | intron1+ 16063 | acctctgtctgctgcagaat A/G aggtgtgatataaatatgtg | 5199 | | |
| OCTN1 | 11 | intron2+ 1105 | atatttccacaaggtccttg C/A gtacactgctccatgctttt | 5200 | | |
| OCTN1 | 12 | intron3+ 1022 | cttctgtcaagttgccagga T/C ggaaatattccaactctact | 5201 | | |
| OCTN1 | 13 | intron3+ 1217 | tccccttcctgcagggggaa G/A gagcggggcaagattttctt | 5202 | | |
| OCTN1 | 14 | intron3+ 1596 | aagccagagaagctctctcc G/A tgggaatgggaacaaggtgg | 5203 | | |
| OCTN1 | 15 | intron3+ 1720 | ggagcctccaagcctcccct G/A tgtgagcgggtgaggcaggg | 5204 | | |
| OCTN1 | 16 | intron3+ 2104 | tatgagactcgttgtgttgg G/A ttctcaggtctgaaagttta | 5205 | | |
| OCTN1 | 17 | intron3+ 8323 | cctttccccttttctaagtg G/C tgatagtttgaactctaact | 5206 | | |
| OCTN1 | 18 | intron4+ 926 | tttttggaactcacaattta G/T actagacctcatggttgccc | 5207 | | |
| OCTN1 | 19 | intron4+ 1055 | cacctgtctgacgagatagc G/A caggtcaggtgggctcactc | 5208 | | |
| OCTN1 | 20 | intron5+ (1197-1202) | caacaacaacaacaacaaca ACAACA/Δ ttgggagtgtctaacacttc | 5209 | | |
| OCTN1 | 21 | intron5+ (2071-2083) | gttctgctcattgctcatgc (T)11-13 caaaaaaagaaactaaggca | 5210 | | |
| OCTN1 | 22 | intron5+ 2781 | tgatcattcctagaaaaaag G/A acactcacatttggagagga | 5211 | | |
| OCTN1 | 23 | intron6+ (882-917) | tcctactctatgatggcagc (AC)15-18 gatgatcgtcagaactggta | 5212 | | |
| OCTN1 | 24 | intron6+ 924 | acacacacacacacgatgat A/C gtcagaactggtagatttag | 5213 | | |
| OCTN1 | 25 | intron7+ 511 | attattgatagtaatagaaa T/C acatatttcttaataataag | 5214 | | |
| OCTN1 | 26 | exon8+ 124 | ggtcaggaacatggcggtgg G/A ggtcacatccacggcctcca | 5215 | | |
| OCTN1 | 27 | intron8+ 3514 | acacacacacctgaaaacat G/A tatgaattctcaggaaaggt | 5216 | | |
| OCTN1 | 28 | intron8+ 3902 | aagcaagatgaggatctgtt T/C ttctcctgtgtgagtaaagc | 5217 | | |
| OCTN1 | 29 | intron8+ (4064-4089) | gtgaacataatacttagttg (T)18-26 gagtctcatagccctgtgga | 5218 | | |
| OCTN1 | 30 | 3′flanking + 115 | aaccaaatgattatatgcag T/A attcctatccagaaaacctt | 5219 | | |
| OCTN2 | 1 | 5′flanking − 225 | cggcgctagaggagcgagtt C/T ggactcggaccccaaggcct | 5220 | | |
| OCTN2 | 2 | 5′flanking − 124 | gctggcagaggccgggcctc G/T ccaggtccccaggacaggcc | 5221 | | |
| OCTN2 | 3 | 5′flanking − 13 | ggcgccgctctgcctgccag C/G ggggcgcgccttgcggccca | 5222 | | |
| OCTN2 | 4 | intron 1 + 232 | ggtggtcagtctggcctccc G/A tcctgatggccactttgaag | 5223 | | |
| OCTN2 | 5 | intron 1 + 314 | atggccctgtgtgtccagga C/T ttactctagttggggttggg | 5224 | | |
| OCTN2 | 6 | intron 1 + 5055 | catgtggtacctagcagcat G/A tctgactgttgatacggtca | 5225 | | |
| OCTN2 | 7 | intron 1 + 6437 | gaagcttggcctcacacaca G/C aggccggcaccctgtcatca | 5226 | | |
| OCTN2 | 8 | intron 2 + (173-174) | tagtaagaagagccaacaaa TC/Δ atctgactccgtaattcttg | 5227 | | |
| OCTN2 | 9 | intron 2 + 608 | agcaggttatttgtataatt C/A taaagcttttaactcaagga | 5228 | | |
| OCTN2 | 10 | intron 2 + 4370 | taatttattgatatccaagt G/A ccctctataatagatgctca | 5229 | | |
| OCTN2 | 11 | intron 5 + 969 | caccagaaaggggtcctgtg C/T gcaaaggtcaggcaggagtg | 5230 | | |
| OCTN2 | 12 | exon 10 + (1028-1044) | ttagcctcctggtttgtgtc (T)16-18 aaaacagaatcactctggca | 5231 | | |
| OCT1 | 1 | intron 1 + 7715 | tagtcctgactcacacatgg G/T tctgtgcttttcgtcctcct | 5232 | | |
| OCT1 | 2 | intron 2 + 97 | ggtggagaacatgaccagtt G/A gaattaactgcagaagctgc | 5233 | | |
| OCT1 | 3 | intron 2 + 797 | gtggagttgtgtgaacaact C/G tttaaaagagtgtggggagg | 5234 | | |
| OCT1 | 4 | intron 2 + 1768 | cgtgaactggagagggtctg T/C gggcactgcccggctgagct | 5235 | | |
| OCT1 | 5 | intron 3 + 1244 | gcagatggtaaaggagcaga C/T gcggaaagcgacggtcaggg | 5236 | | |
| OCT1 | 6 | intron 4 + 865 | agcgtccagtggtaggaaag G/T ctccacaggtggcaatccca | 5237 | | |
| OCT1 | 7 | intron 4 + 1028 | gtcatctctgctcttctccc A/G cttcttcatttttatagtac | 5238 | | |
| OCT1 | 8 | intron 4 + 1040 | cttctcccacttcttcattt T/G tatagtactattggtattat | 5239 | | |
| OCT1 | 9 | intron 4 + 1485 | agcctgcccttcccctgcct C/T gtccttgtgaaacagggatc | 5240 | | |
| OCT1 | 10 | intron 4 + 1997 | tgagggattacagccccaac G/A tggggagggcaggctgcact | 5241 | | |
| OCT1 | 11 | exon 5 + 9 | tggtgttcgcaggtgtgtgc C/T ggagtcccctcggtggctgt | 5242 | | |
| OCT1 | 12 | exon 5 + 20 | ggtgtgtgccggagtcccct C/G ggtggctgttatcacaaaaa | 5243 | | |
| OCT1 | 13 | intron 6 + 379 | gaggaagttccattcctcat A/G tctaaacaccctagagaccc | 5244 | | |
| OCT1 | 14 | intron 6 + 2125 | tattgacccaaatctgttct C/A acaatgtaaatatgactgta | 5245 | | |
| OCT1 | 15 | intron 6 + (2935-2953) | tttctccatctgcgaggggc (T)18-20 cttcagtctctgactcatgc | 5246 | | |
| OCT1 | 16 | intron 7 + (6-7) | ttttatctcacctggtaagt (TGGTAAGT) tggtaagttgtctgctttc | 5247 | | |
| OCT1 | 16 | intron 7 + (6-7) | ttttatctcacctggtaagt ( tggtaagttgtctgctttc | 5248 | | |
| OCT1 | 17 | intron 7 + (1780-1781) | gttttcttttcccttttttt (T) catggagaaagaacagagaa | 5249 | | |
| OCT1 | 17 | intron 7 + (1780-1781) | gttttcttttcccttttttt catggagaaagaacagagaa | 5250 | | |
| OCT1 | 18 | intron 8 + 3247 | ccaggccaaacaattccatt G/T tcatggccactgggccaagg | 5251 | | |
| OCT1 | 19 | intron 8 + 10521 | cccttaaccaatgaacgcca G/A tggcagatccctcattctga | 5252 | | |
| OCT1 | 20 | intron 10 + 393 | tcagattctttagtaacttt G/C ttcacaaaattcttttgaca | 5253 | | |
| OCT1 | 21 | 3′flanking + 1755 | tgaatgatgtttttcaaatg T/C gtattaaaaatgtcctctct | 5254 | | |
| OCT1 | 22 | 3′flanking + 1799 | ctttcttagaatcctcttgg G/Δ caaaacttctgaggaaggcc | 5255 | | |
| OCT2 | 1 | intron 2 + 1329 | tggcagcagaagggaagagg G/Δ ataaaagtggaggcacaggc | 5256 | | |
| OCT2 | 2 | intron 2 + 1867 | cctctgtcaaggtaagtact C/Δ attattcttcccccaaaggc | 5257 | | |
| OCT2 | 3 | intron 9 + (340-343) | cagcaggcccctaactctct CTCT/Δ gctgatttccacccttcctg | 5258 | | |
| OCT2 | 4 | intron 9 − 396 | atacataattcattactttt A/G tttgctagaaatgatccaag | 5259 | | |
| OCT2 | 5 | intron 9 − 386 | cattacttttatttgctaga A/C atgatccaagtttctgactt | 5260 | | |
| OCT2 | 6 | intron 9 − 86 | atagaaaaatgctaaaaaaa A/Δ gttttaaacaaaaataaggg | 5261 | | |
| OCT2 | 7 | intron 10 + 1725 | tggaagaggcctttgaatcc G/A agcggaggtcacacactcgc | 5262 | | |
| OCT2 | 8 | intron 10 − 195 | caagataattttaggaataa C/T tctgtcgacatgagttatca | 5263 | | |
| OCT2 | 9 | exon 11 + 328 | gttttctggagggttttttt T/Δ ccatctttgtatttttttaa | 5264 | | |
| OCT2 | 10 | exon 11 + 427 | aggcaaacaaaatagaaaaa A/T gtgtgaaaaacagtaaagtt | 5265 | | |
| OCT2 | 11 | exon 11 + 455 | aaacagtaaagttgggagag G/A agcatctattttcttaaaga | 5266 | | |
| OCT2 | 12 | 3′flanking + 34 | agaatgtatgtcaagaattt T/A agataggcctttcagtaaca | 5267 | | |
| NTCP | 1 | exon 1 + 307 | tatggcatcatgcccctcac G/A gcctttgtgctgggcaaggt | 5268 | | |
| NTCP | 2 | intron 1 + 607 | cccagcacccactccagata G/C gccagccccatctcagccac | 5269 | | |
| NTCP | 3 | intron 1 + 702 | gcagaaatcagcaagggctc G/A ctcctggagacgcagcacac | 5270 | | |
| NTCP | 4 | intron 1 + (3950-3966) | cacatcacctaacagcttgc (T)14-17 gagaaataggcatgtaaaga | 5271 | | |
| NTCP | 5 | intron 1 + 5597 | aaggacatattattcaggct C/G tgagtgtcataatttatttt | 5272 | | |
| NTCP | 6 | intron 2 + 4808 | cctatggagaagcaactacc C/T ggggccacttgtctcagcag | 5273 | | |
| NTCP | 7 | intron 2 + 5032 | acacctggagactagcagag G/C cagctttcccaccaggatca | 5274 | | |
| NTCP | 8 | intron 2 + 5046 | gcagaggcagctttcccacc A/T ggatcatatcaaattatgtg | 5275 | | |
| NTCP | 9 | intron 3 + (8-21) | gcctcaatggacggtaggta (T)12-15 aagaaagggtctcactctgt | 5276 | | |
| NTCP | 10 | intron 4 + (484-495) | taatataacccagaataaag (A)10-13 gattcctcaactctagttac | 5277 | | |
| NTCP | 11 | intron 4 + (728-754) | tgcactttaacaccaatttt (A)25-27 caggacattcaaacccactt | 5278 | | |
| NTCP | 12 | intron 4 + 747 | taaaaaaaaaaaaaaaaaaa A/C aaaaaaacaggacattcaaa | 5279 | | |
| NTCP | 13 | intron 4 + 1339 | ccccagtggaaacactaaat C/A aaagcaacgtatttctttgg | 5280 | | |
| NTCP | 14 | intron 4 + 1545 | accacggacaagaagaggta G/C atcaattgggggttggaggg | 5281 | | |
| NTCP | 15 | 3′flanking + 559 | caagacaatatagttttcgg G/A tatcagtttggcaaatgtgc | 5282 | | |
| PEPT1 | 1 | exon 1 + 25 | ctgccaggagcacgtcccgc C/T ggcaggtcgcaggagccctg | 5283 | | |
| PEPT1 | 2 | intron 1 + 88 | cgagggccgggaggcgcgaa G/A ggtacgcggcggcgggaagc | 5284 | | |
| PEPT1 | 3 | intron 1 + 106 | aagggtacgcggcggcggga A/T gcggggcgacccgaaggccc | 5285 | | |
| PEPT1 | 4 | intron 1 + 248 | cgaggttgcgatcctggccc G/A cccgcccgtggggcactgta | 5286 | | |
| PEPT1 | 5 | intron 3 + 326 | tggagcgggacgggacccag C/A gggtgacggcaggggcggca | 5287 | | |
| PEPT1 | 6 | intron 1 + 1238 | tttagcatttccagcagatc C/T aatcccgagagctgttagag | 5288 | | |
| PEPT1 | 7 | intron 1 + 3001 | tcttatatgctgggaagaag C/T gtcagtaagaaaaagcagcc | 5289 | | |
| PEPT1 | 8 | intron 1 + 5673 | ttgggaagtgccacagccac G/C gggcacagggacagggtctt | 5290 | | |
| PEPT1 | 9 | intron 1 + 5679 | agtgccacagccacggggca C/G agggacagggtcttccacag | 5291 | | |
| PEPT1 | 10 | intron 1 + 5917 | aaattcacaaaatgtacttc C/T ataagaaggctcgttaaaag | 5292 | | |
| PEPT1 | 11 | intron 1 + 5966 | ctaggcatttagaacttcta C/T aatctgcccctagtgacaag | 5293 | | |
| PEPT1 | 12 | intron 1 + 9255 | tggtcatttcaggcctcttc A/G gcctatgattttagatagtt | 5294 | | |
| PEPT1 | 13 | intron 1 + 10278 | catgacccatgtaggcggga A/G aagcagccctgtagcagcag | 5295 | | |
| PEPT1 | 14 | intron 1 + 20251 | aagaagagcctgtgtttatt C/T agtgattgcaatgtgttggg | 5296 | | |
| PEPT1 | 15 | intron 1 + 20509 | aaacaccacttctgcatttg C/A gctttctaagatagcaatcc | 5297 | | |
| PEPT1 | 16 | intron 1 + 20532 | tttctaagatagcaatcctg T/C tgacacaggtacattaagat | 5298 | | |
| PEPT1 | 17 | intron 3 + 55 | agagcgggagtggccataac C/Δ agtcctaactttgtttcccc | 5299 | | |
| PEPT1 | 18 | intron 5 + 1720 | atcctctcttttactggaaa C/A aataaagctacaaaagaacc | 5300 | | |
| PEPT1 | 19 | intron 5 + 1790 | gctactgttttatgttttcc G/A gatggtaaattattagatgg | 5301 | | |
| PEPT1 | 20 | intron 5 + 1860 | agtttgcatttgactatcac G/A ctgcattcctgtgagctggc | 5302 | | |
| PEPT1 | 21 | intron 5 + 1943 | aggcccactgagggaaactg G/A ggaaaagagaggccttctac | 5303 | | |
| PEPT1 | 22 | intron 8 + 1478 | tgttttcagatcttagtagt A/G catggaataggaccgttttc | 5304 | | |
| PEPT1 | 23 | intron 8 + 1898 | ttaaatattagtggtaaaag A/G aaacatagactcaatctctt | 5305 | | |
| PEPT1 | 24 | intron 10 + 388 | ttaaatagtttagacatttt C/T gattttctaaagaaaactgc | 5306 | | |
| PEPT1 | 25 | intron 11 + 985 | atccataaggtactcagtga C/T tggcctgtatgaagaactca | 5307 | | |
| PEPT1 | 26 | intron 11 + (1022-1045) | ctcaaacaggggtagatttc (T)20-24 gagtcaagagtctcactctg | 5308 | | |
| PEPT1 | 27 | intron 11 + 1320 | tgtgagccactgcacctggc C/T aatttcctgactttctatga | 5309 | | |
| PEPT1 | 28 | exon 16 107 | tggagagatggtgacacttg G/C cccaatgtctcaagtaagta | 5310 | | |
| PEPT1 | 29 | intron 18 + 6048 | tttgttgttgggtttttttt T/Δ gttgttgttgttttgttttg | 5311 | | |
| PEPT1 | 30 | intron 18 + (6141-6142) | tcactgcagcctccgccccc (T) gggttcaagcaattatcctg | 5312 | | |
| PEPT1 | 30 | intron 18 + (6141-6142) | tcactgcagcctccgccccc gggttcaagcaattatcctg | 5313 | | |
| PEPT1 | 31 | intron 18 + (6241-6242) | tatttttagtagagacgggg (G) tttcaccatattggccaggc | 5314 | | |
| PEPT1 | 31 | intron 18 + (6241-6242) | tatttttagtagagacgggg tttcaccatattggccaggc | 5315 | | |
| PEPT1 | 32 | intron 18 + 12102 | gtgggaattctagctaaggc C/T cgtgtggatctgtctcaggt | 5316 | | |
| PEPT1 | 33 | intron 18 + 12203 | gacctgagtttaattcatag C/A cattttctcccagcacctaa | 5317 | | |
| PEPT1 | 34 | intron 18 + 12307 | gaaaggttaaattattcttt A/G cactgctgaggtgtacacta | 5318 | | |
| PEPT1 | 35 | intron 20 + 79 | tcacaaacacttaggacata A/G tatgatttaactagagtgat | 5319 | | |
| PEPT1 | 36 | exon 23 + (348-370) | tcttttttctttttcttttc (T)18-23 gagacagagttttgctcttg | 5320 | | |
| PEPT1 | 37 | exon 23 + 790 | ccacattggtcatcttccct A/G tcacacaaatgatgttattt | 5321 | | |
| PEPT1 | 38 | 3′flanking + 2 | aaataaatttctgttcttaa G/A cctaagtgttcatgtatctc | 5322 | | |
| EPHX1 | 1 | intron 1 + 110 | tgcaaaatgtgtcttactag C/T ttctagtgcataaaatattg | 5323 | | |
| EPHX1 | 2 | intron 1 + 143 | aaatattggtggagctcttc G/A ctgtgctgggccagtcacca | 5324 | | |
| EPHX1 | 3 | intron 1 + 1097 | aatccagagagggagataga T/G tggaagttcaagggtggaca | 5325 | | |
| EPHX1 | 4 | intron 1 + 1717 | ttccaagacagagcgagggg T/C gctgctggggcgtggtttgc | 5326 | | |
| EPHX1 | 5 | intron 1 + 1772 | aactcgatgctttctcctcc G/T tctgggtcctaactgcagtg | 5327 | | |
| EPHX1 | 6 | intron 1 + 2054 | gaaatgtaacaggcaacact A/G tggacacagaaagtagatta | 5328 | | |
| EPHX1 | 7 | intron 2 + 1414 | atttccaaaatctgtttggg G/T gtaactgaaacacttgggaa | 5329 | | |
| EPHX1 | 8 | exon 3 + 174 | taccctcacttcaagactaa G/A attgaaggtatgtttgcaaa | 5330 | | |
| EPHX1 | 9 | intron 3 + 6583 | ctgtcaataccatgaagggg G/C ggcgggggcactaagggtgg | 5331 | | |
| EPHX1 | 10 | intron 4 + 34 | agaggttccataactgcccc G/A tcctcgccaagggtgggccc | 5332 | | |
| EPHX1 | 11 | intron 4 + 63 | aagggtgggcccggtgttcc C/T accaggctctccttccggcg | 5333 | | |
| EPHX1 | 12 | intron 5 + 154 | gcagtgcctgaggcacgttg G/A cttggatcctcctgtctgta | 5334 | | |
| EPHX1 | 13 | intron 5 + 276 | tgctggaccaagctctggga T/C agccctgagcagaactcccc | 5335 | | |
| EPHX1 | 14 | exon 6 + 130 | gatgtggagctgctgtaccc C/T gtcaaggagaaggtattcta | 5336 | | |
| EPHX1 | 15 | intron 8 + 206 | ggtgcctggctcccgggcgg C/A cctcagtaccgctccccagt | 5337 | | |
| EPHX1 | 16 | intron 8 + 353 | tggccctcccagaaaagaga A/G ggccctcagtgaggggagag | 5338 | | |
| EPHX1 | 17 | 3′flanking + 708 | aggtgcagactcatgcactc A/G gccctgaagaggtgagagag | 5339 | | |
| EPHX2 | 1 | 5′flanking − (523-522) | aaagtcactggatatgcccc (C) tcccccgccccccaacacgg | 5340 | | |
| EPHX2 | 1 | 5′flanking − (523-522) | aaagtcactggatatgcccc tcccccgccccccaacacgg | 5341 | | |
| EPHX2 | 2 | 5′flanking − 522 | aaagtcactggatatgcccc T/C cccccgccccccaacacggt | 5342 | | |
| EPHX2 | 3 | 5′flanking − 521 | aagtcactggatatgcccct C/T ccccgccccccaacacggtc | 5343 | | |
| EPHX2 | 4 | 5′flanking − 516 | actggatatgcccctccccc G/C ccccccaacacggtcttatg | 5344 | | |
| EPHX2 | 5 | 5′flanking − 515 | ctggatatgcccctcccccg C/G cccccaacacggtcttatgt | 5345 | | |
| EPHX2 | 6 | intron 1 − 74 | tggctgcttctcaatgaata T/C gaacagtgtctgtttccatg | 5346 | | |
| EPHX2 | 7 | intron 3 + 72 | gagcattaggtcagaatcca T/C tgaagtgagctttgagatca | 5347 | | |
| EPHX2 | 8 | intron 4 + 473 | gtgtgtctctactttaatct A/G caaaaggtgattgaatggag | 5348 | | |
| EPHX2 | 9 | intron 5 + 276 | caagagtgggatgttcaagg C/T catcctgacctcacttttga | 5349 | | |
| EPHX2 | 10 | intron 8 + 8 | tctgctcctcccggtgggtg T/C gctgtcttgcagctgtctta | 5350 | | |
| EPHX2 | 11 | intron 9 + 1573 | atgtcgtgaagactgatgaa C/T gatggacggctgcactgctc | 5351 | | |
| EPHX2 | 12 | intron 10 + 207 | gaacaggatggagatgagct T/C gtttatttgtcttttaatga | 5352 | | |
| EPHX2 | 13 | intron 12 + 911 | tgaagagacctcgacatgtc G/T catcccacatactacaggga | 5353 | | |
| EPHX2 | 14 | intron 12 + 2425 | atcttctcagctgagcaaac C/T gaggctcagagggcttaacc | 5354 | | |
| EPHX2 | 15 | intron 12 + 2460 | ttaaccccaactggcccaag G/A ccaggtacatgattgggtca | 5355 | | |
| EPHX2 | 16 | intron 12 − 281 | aagtcctttcaagagattat T/C ataagtagtaccttctcatt | 5356 | | |
| EPHX2 | 17 | intron 12 − 268 | agattattataagtagtacc T/G tctcattataggaatattga | 5357 | | |
| EPHX2 | 18 | exon 13 + 50 | cctgagtcggactttcaaaa G/T cctcttcagagcaagcgatg | 5358 | | |
| EPHX2 | 19 | intron 13 + 1739 | ttgtcgtaacagggttttca G/T atgagcatatttcctttgta | 5359 | | |
| EPHX2 | 20 | exon 14 + 33 | atgcataaagtctgtgaagc G/A ggtaagagacatgcttggga | 5360 | | |
| EPHX2 | 21 | intron 14 + 314 | ggattgagagcttacctcta T/C gggggtcacctcgtgtatgc | 5361 | | |
| EPHX2 | 22 | intron 14 + 878 | attcccttattccttcacac C/T gtctgtcactcattcattca | 5362 | | |
| EPHX2 | 23 | intron 14 + 948 | gcacaggctgggtatgaagc T/C ggggctgcatgctcagctac | 5363 | | |
| EPHX2 | 24 | intron 15 + 259 | agagggttttcactactttt C/T agtcatggctcctcagagaa | 5364 | | |
| EPHX2 | 25 | intron 16 + 459 | tcctcatttgtcaagcagaa G/C atgagtttccaatctctggg | 5365 | | |
| EPHX2 | 26 | intron 16 + 645 | gtaagtgaacacactgctac G/A tgccagacttcctgccagac | 5366 | | |
| EPHX2 | 27 | intron 16 + 985 | gtcattatcatcatatgacc G/A atgaaaatgaccaaactgca | 5367 | | |
| EPHX2 | 28 | 3′flanking + 12 | aggtggccttacacacatct T/C gcatggatggcagcattgtt | 5368 | | |
| EPHX2 | 29 | 3′flanking + 374 | tgttcacggagaatgcacgg C/T atggggatgaaccctttccc | 5369 | | |
| EPHX2 | 30 | 3′flanking + 544 | tagccacctgcctttctccc G/A gcttccctagcagagtttgc | 5370 | | |
| COMT | 1 | 5′flanking + 1287 | cgtatgatattccccattct G/A agtccagaatacctagaaat | 5371 | | |
| COMT | 2 | 5′flanking + 1217 | tgtgagtatgggaaggggaa G/A cttttctgtctgttgtcccc | 5372 | | |
| COMT | 3 | 5′flanking − 503 | caggggctccaggaggacga G/A tgtgtatcctcccattgctc | 5373 | | |
| COMT | 4 | 5′flanking − 425 | gagaagttgggaagtctggc C/T agtggggccggtgcctggtg | 5374 | | |
| COMT | 5 | 5′flanking − 277 | cccagccccagtttccccac C/T tgggaagggggctacttgtg | 5375 | | |
| COMT | 6 | intron 1 + 12058 | ctggcccatggaagggaggg G/A agggggccccgacggggcca | 5376 | | |
| COMT | 7 | intron 1 + 12070 | agggaggggagggggccccg A/G cggggccacagtaaaggagt | 5377 | | |
| COMT | 8 | intron 1 + 18831 | tgtgtatgttcttggtaaac C/T agcccttggtcttacacatc | 5378 | | |
| COMT | 9 | intron 2 + 832 | cctctcctttggccacccgt G/C actacccccaactccgggcc | 5379 | | |
| COMT | 10 | intron 3 + 90 | ggagaagctgttatcacccc A/G cggggccacagtaaaggagt | 5380 | | |
| COMT | 11 | intron 3 + 425 | ccccaaggtgggcggttcgg T/G gattcagagagggcagctct | 5381 | | |
| COMT | 12 | intron 3 + 671 | ggctcctgctctttgggaga G/A gtggggggccgtgcctgggg | 5382 | | |
| COMT | 13 | intron 3 + 676 | ctgctctttgggagaggtgg G/T gggccgtgcctggggatcca | 5383 | | |
| COMT | 14 | intron 5 + 75 | tcagcctcagcctctccaaa G/C agccaggcattccagtagag | 5384 | | |
| COMT | 15 | intron 5 + 310 | accagacaccagggcagaaa C/T ggcacaggaccaaggagatg | 5385 | | |
| COMT | 16 | intron 5 + 346 | agatggggtggggaagggcc G/A ctctgggcccagcctgctct | 5386 | | |
| COMT | 17 | intron 5 + 3023 | aaggcagccgccctgctcaa G/A gcctaggccattgtcctcct | 5387 | | |
| GAMT | 1 | intron 1 + 429 | ctcggaaagctgagctcagg G/A agacagctgtccccggggtg | 5388 | | |
| GAMT | 2 | intron 5 + 1411 | ggtgacctggtgccatcccc G/A accaggagacgcaggtgccc | 5389 | | |
| GAMT | 3 | 3′flanking + 626 | cactgacctccttgccctga G/A agaaggccggctcctgtgct | 5390 | | |
| PNMT | 1 | intron 1 + 429 | ctcggaaagctgagctcagg G/A agacagctgtccccggggtg | 5391 | | |
| PNMT | 2 | intron 1 + 35 | ctgaggcacgagggacaaga G/T gtcgtcgggggatgaaagca | 5392 | | |
| HNMT | 1 | 5′flanking − 211 | cagaggcagatgacagtctt C/T cgttaaagatttcactgctg | 5393 | | |
| HNMT | 2 | intron 1 + 5409 | aatataactgatataattgg A/G acatttcatgttggcctagt | 5394 | | |
| HNMT | 3 | intron 2 + 2561 | cacttgtgcttggacaagaa A/G agaaggcctacaagaaaaag | 5395 | | |
| HNMT | 4 | intron 2 + 2895 | caatcagaaatgtaagaaaa A/C ctccaagaaaaatttaagtt | 5396 | | |
| HNMT | 5 | intron 2 + 3977 | accaaacttggaagtgtaaa G/A ttatgcatgtatgttcatgt | 5397 | | |
| HNMT | 6 | intron 2 + 5296 | ttaacatagtgagtttggag T/C cccaggattttattttcctt | 5398 | | |
| HNMT | 7 | intron 2 + 13317 | caaccctcatgaattcttag C/T tgggatgggtccctataaca | 5399 | | |
| HNMT | 8 | intron 2 + 14682 | gtagatgagcaaatgagttc A/Δ ggagagatttaaatacccta | 5400 | | |
| HNMT | 9 | intron 2 + 15406 | gtctatgcattcatgcatcc G/A tctaaccagctgtctaccta | 5401 | | |
| HNMT | 10 | intron 2 + 28943 | atgtgacttaaacttcaggt A/G tatcaatatcccttgaatgt | 5402 | | |
| HNMT | 11 | intron 4 + 49 | cagaaagaagacttttcaga A/G tatatatataatgaatatct | 5403 | | |
| HNMT | 12 | intron 4 + (1942-1943) | tttgagaaaaatttaaggta (A) tcttctatggcccacttcca | 5404 | | |
| HNMT | 12 | intron 4 + (1942-1943) | tttgagaaaaatttaaggta tcttctatggcccacttcca | 5405 | | |
| HNMT | 13 | intron 4 + 2405 | ccctgtgaccaagcagataa C/A ctcatgctttatttagtcca | 5406 | | |
| HNMT | 14 | intron 5 + (80-81) | cctgtgtttgaaagaagctt (TT) atatattttgtcttcattat | 5407 | | |
| HNMT | 14 | intron 5 + (80-81) | cctgtgtttgaaagaagctt atatattttgtcttcattat | 5408 | | |
| HNMT | 15 | intron 5 + 235 | ctttcttttgggaaaatatg T/C ctttgtcttctatatatgaa | 5409 | | |
| HNMT | 16 | intron 5 + (702-703) | tacttacaggttgattttag (AT) acacagcagactctgtcttc | 5410 | | |
| HNMT | 16 | intron 5 + (702-703) | tacttacaggttgattttag acacagcagactctgtcttc | 5411 | | |
| HNMT | 17 | intron 5 + 749 | ttacaccagaccccatactt T/G aacaccatatgtcacaaaat | 5412 | | |
| HNMT | 18 | intron 5 + 1101 | gtaggcagcctattcttgat T/G atattcatcaatcatacaga | 5413 | | |
| HNMT | 19 | intron 5 + 1137 | acagaaaaagtattgtagac G/A gaaataacaattcattgaga | 5414 | | |
| HNMT | 20 | intron 5 + 1348 | aagggagcatgaatagtcca C/G aagtaactgagaactgatta | 5415 | | |
| HNMT | 21 | intron 5 + 1673 | caaaagaaagggagtaaaga C/G tcaacaatcagttagctttt | 5416 | | |
| HNMT | 22 | intron 5 + 2022 | attttatttggggctttcta C/T gtctctctctcctaagccta | 5417 | | |
| HNMT | 23 | intron 5 + 2285 | tgtcatacttaactcttaaa G/C atccagagtaaatgatggag | 5418 | | |
| HNMT | 24 | intron 5 + 4159 | taccagttgacccagcaacc C/T tcttatagagtagtttaaat | 5419 | | |
| HNMT | 25 | intron 5 + 4501 | aatgatccacaaaattacta C/G tcattgttttctttcaatga | 5420 | | |
| HNMT | 26 | intron 5 + 5251 | cacacacacacacacacaca C/G caaatggaagcagccagaca | 5421 | | |
| HNMT | 27 | intron 5 + 5802 | gaaaaagaaaatctggctta C/T tataagaccaagagtcatct | 5422 | | |
| HNMT | 28 | intron 5 + 6189 | tccaattccaccttctccta G/C agcatatcctgcagttacct | 5423 | | |
| HNMT | 29 | intron 5 + 6297 | gtcttggttcatctcttgag T/A taaattagatctgggaactt | 5424 | | |
| HNMT | 30 | 3′flanking + 458 | tatgtcactctcaagaactc C/T tataagaccaagagtcatct | 5425 | | |
| HNMT | 31 | 3′flanking + 993 | ctgaaaatgaacactgaacc G/A ttaatcatactgatatgtac | 5426 | | |
| HNMT | 32 | 3′flanking + 1793 | gtggagcacagcattttagg G/A cttgatatttgcttattata | 5427 | | |
| NNMT | 1 | 5′flanking − 228 | ataattttcctgacgagctc A/T agtgctccctctggtctaca | 5428 | | |
| NNMT | 2 | intron 1 + 44 | ccccactaatgtgagtcata T/C agatggagtctcagggcacg | 5429 | | |
| NNMT | 3 | intron 1 + 149 | ggataaaaacgaatattggt A/G tagcgattccacagtttaca | 5430 | | |
| NNMT | 4 | intron 2 + 158 | agataggcccatgtgtgtgc G/A tgttagtaaatttgtgtatg | 5431 | | |
| NNMT | 5 | intron 2 + 433 | gctgtagccatccaagccta T/C agaacttggctgtgagtgtg | 5432 | | |
| NNMT | 6 | intron 2 + 10826 | atcatctgactggtaagttc C/T agttctgtggtaactcaagt | 5433 | | |
| NNMT | 7 | intron 2 + 13630 | atttcatggagggaagtcca T/C ggtagaagcaggctgctagg | 5434 | | |
| NNMT | 8 | 3′flanking + 71 | ggctcagtggttggggccca A/G tggttcatctaggacgggac | 5435 | | |
| PEMT | 1 | intron 1 + (297-299) | attgtgtgagactcagaggt TGT/Δ ccgtgttagtctttgggatt | 5436 | | |
| PEMT | 2 | intron 1 + 817 | tcatgaagcctgtaaggcac A/G tctctgccccaagcagcttc | 5437 | | |
| PEMT | 3 | intron 1 + 830 | aaggcacatctctgccccaa G/A cagcttctaatccagttctt | 5438 | | |
| PEMT | 4 | intron 1 + 1035 | gagttctctgaaggagctaa T/C accagttagtgttttgaaga | 5439 | | |
| PEMT | 5 | intron 1 + 1573 | agtgggcaggggagactaac C/T gggtgtgtgaggggtgggct | 5440 | | |
| PEMT | 6 | intron 1 + 1759 | gatttttcttaaagaaagaa A/G gaaagaaacatacaacatac | 5441 | | |
| PEMT | 7 | intron 1 + 2768 | gcatcttgctgtccacaggc C/A ggggcacctccaggattcag | 5442 | | |
| PEMT | 8 | intron 1 + 2785 | ggccggggcacctccaggat T/C cagaagatgactccagtagg | 5443 | | |
| PEMT | 9 | exon 1 + 162 | agctcagcagacctcctggc C/T gtggtgggtagctcctttcc | 5444 | | |
| PEMT | 10 | intron 2 + 4598 | ccgtgggttttttttttttt t/Δ cttcatttctttggttgctg | 5445 | | |
| PEMT | 11 | intron 4 + 39 | actgtccagacgggagtatc C/T cactgcttggtgagccccac | 5446 | | |
| PEMT | 12 | intron 4 + 1317 | accgtccccagctggcccca G/A cctcctgacatgggcctctg | 5447 | | |
| PEMT | 13 | intron 4 + 1355 | ctggagccaggctgcagccg A/C agtgcctggccatcctggcg | 5448 | | |
| PEMT | 14 | intron 4 + 5925 | gtccaggcactgtggcccta C/T gtgggagtctccagtctcca | 5449 | | |
| PEMT | 15 | intron 4 + 6028 | ggcagtggtccaaggaccag G/C atggactccctcttctcacc | 5450 | | |
| PEMT | 16 | intron 4 + 6078 | atctgtaccctcgcggactc C/T acctggcttcgtgccatcac | 5451 | | |
| PEMT | 17 | intron 4 + 6089 | cgcggactctacctggcttc A/G tgccatcacccccgccagat | 5452 | | |
| PEMT | 18 | intron 4 + 6379 | tcaggtgtcccctccctcat G/A cctcctcaccctgccctctc | 5453 | | |
| PEMT | 19 | intron 4 + 7339 | tgtaaggaatcctgccaaga C/T ggcagatgcacacggggtca | 5454 | | |
| PEMT | 20 | intron 4 + 7619 | ctcctgcacatgtgctccag A/G gaggaaaggcatttgacagg | 5455 | | |
| PEMT | 21 | intron 4 + 8858 | ggcatgtgtgtgtgtgtgta T/G gtgtgtgagtgtgtgcatgt | 5456 | | |
| PEMT | 22 | intron 4 + 9029 | tttctggaccagaaagcgtc G/A tcctctgccagggcctcttg | 5457 | | |
| PEMT | 23 | intron 4 + 9056 | gccagggcctcttgcacttg C/T gggaaagctgagctgagctg | 5458 | | |
| PEMT | 24 | intron 4 + 9512 | ctgagctgggcagcagcatt A/G ctctgtgtgctgctggcact | 5459 | | |
| PEMT | 25 | intron 4 + 9523 | agcagcattactctgtgtgc T/C gctggcactggcctggtggg | 5460 | | |
| PEMT | 26 | intron 4 + 9622 | gacaaagtgtacaacaaggt G/A tctcgaactgggtcagctca | 5461 | | |
| PEMT | 27 | intron 4 + 10776 | ccattcctgggtcttctttg G/A aggctgaatgaaattccatg | 5462 | | |
| PEMT | 28 | intron 4 + 10912 | tctgccccactttgctcaga G/C gtgcaacaaggccttcagga | 5463 | | |
| PEMT | 29 | intron 4 + 11590 | ggacactggcctgatgcaga G/C gtgtggtctctctcctgcag | 5464 | | |
| PEMT | 30 | intron 4 + 12090 | ggccagggcacccctaccag G/C ctgagtcccacctgtccagc | 5465 | | |
| PEMT | 31 | intron 4 + 12263 | tacccgccttcccagatgga G/A cgggctgctcatgggactta | 5466 | | |
| PEMT | 32 | intron 4 + 12448 | tctggtcccctctcctgctt G/A tagtttcctgggctaaaatc | 5467 | | |
| PEMT | 33 | intron 4 + 12730 | tgggaccagtgccgccacca C/T ggcccaaggacctggtgttc | 5468 | | |
| PEMT | 34 | intron 4 + 13240 | gggctccaggcacacagcgg T/C cccagtacacctgtcgcttt | 5469 | | |
| PEMT | 35 | intron 4 + 13494 | tccgtggaactcagagatgg T/C acctccctgcgaggtggggc | 5470 | | |
| PEMT | 36 | intron 4 + 13817 | aactctcccctgctgctgag A/G cagatcttggagcctcggcc | 5471 | | |
| PEMT | 37 | intron 4 + 14773 | ccgccctgtgcttcatgccc C/T ctatgcctctcactgcctgg | 5472 | | |
| PEMT | 38 | intron 4 + 14951 | gtcctgaggcccctcccacc G/A ggggcttctcaaccacacac | 5473 | | |
| PEMT | 39 | intron 4 + 16896 | gctgtgactgtcttggagac T/C gggtcttggcgggcctggtg | 5474 | | |
| PEMT | 40 | intron 4 + 19439 | ccaggagcctctgaggcagc G/A ggggcttctcaaccacacac | 5475 | | |
| PEMT | 41 | intron 4 + 19557 | attttgtcagcatgtcacgt C/T cctttcataatgaagcaagg | 5476 | | |
| PEMT | 42 | intron 4 + 20051 | acagcactgcgggagccacg A/G catctgcagacgcatttgat | 5477 | | |
| PEMT | 43 | intron 4 + 20816 | tggactctctggcgtccatc C/T agccacttcagtgcgacgtg | 5478 | | |
| PEMT | 44 | intron 4 + 21196 | ggctggctgggccctgggat C/G atcgtgacaggctttagtgg | 5479 | | |
| PEMT | 45 | intron 4 + 21528 | acaggtgggagccgaggctc G/T ggaggtgggccgggctgagc | 5480 | | |
| PEMT | 46 | intron 4 + 21596 | ccgcttccccgtgctctggc C/T gtagcagaaagtgtcccact | 5481 | | |
| PEMT | 47 | intron 4 + 22672 | agcctcccactgccttgtgg C/T tgaggggagggggccgggtc | 5482 | | |
| PEMT | 48 | intron 4 + 22713 | tctaacgctgtcttctttgt A/T ctgaaaaccaaacaccttct | 5483 | | |
| PEMT | 49 | intron 4 + 23010 | tgccgggcagcggggaggga G/A ggcgagtggttcccccaagt | 5484 | | |
| PEMT | 50 | intron 4 + 23588 | gtgcaggcgccctgcatccc C/T gcagccaagttctgggcgga | 5485 | | |
| PEMT | 51 | intron 4 + 23627 | gacactgccctgagccagga C/T ggtgaggtgggacgccttcc | 5486 | | |
| PEMT | 52 | intron 4 + 23941 | tgaggggttgggactctaca G/A aggagagtggactcacgggg | 5487 | | |
| PEMT | 53 | intron 4 + 24091 | gacacctcttcactgtcagc G/T ctgagacacgcccctgccct | 5488 | | |
| PEMT | 54 | intron 4 + 25348 | caggccagttggaatcctac G/A tagagtgaaagcatctcagc | 5489 | | |
| PEMT | 55 | intron 4 + 25603 | taagcagttaacactgatgc G/A tgatgaaaattccaacagca | 5490 | | |
| PEMT | 56 | intron 4 + 31540 | cctccaggtggcaggaacac T/C gtgaggagcatgcaacgtgc | 5491 | | |
| PEMT | 57 | intron 4 + 31637 | gtgggctgggacgccaggac G/A gtgaggggcttcaaggtgtg | 5492 | | |
| PEMT | 58 | intron 4 + 31642 | ctgggacgccaggacggtga G/A gggcttcaaggtgtgtttgt | 5493 | | |
| PEMT | 59 | intron 4 + 35593 | ggaggagctgaaagagctgg G/A gctcgggatcaggtggttca | 5494 | | |
| PEMT | 60 | intron 4 + 35647 | actttgaggcaccaccgcac C/A tgtccgtgcgtgagggagac | 5495 | | |
| PEMT | 61 | intron 4 + 35862 | tcccagtggtggctctgtcc C/T cgtctcagccgagcactcag | 5496 | | |
| PEMT | 62 | intron 4 + 35882 | ccgtctcagccgagcactca T/G cggccagggtggctggactc | 5497 | | |
| PEMT | 63 | intron 4 + 37141 | ccacaggccggatgccttga T/C acttctcagctgcagggctg | 5498 | | |
| PEMT | 64 | intron 4 + 38862 | tggagagaccacctcagaca C/G caaggacgggcatgccatgg | 5499 | | |
| PEMT | 65 | intron 4 + 38872 | acctcagacagcaaggacgg G/T catgccatgggtcccggcag | 5500 | | |
| PEMT | 66 | intron 4 + 39140 | atgtctcaaatctccctccc C/T gggaaatctaggcacaggtc | 5501 | | |
| PEMT | 67 | intron 4 + 39635 | caggcccaggagcaggtggg G/T cctcctcacaggagcagggc | 5502 | | |
| PEMT | 68 | intron 4 + 39713 | actctgagcatgctggctcc C/T tccttctttccagggcagca | 5503 | | |
| PEMT | 69 | intron 4 + 40436 | cctggttgtgcttcggaccc G/A gaggcagacagaggaggcct | 5504 | | |
| PEMT | 70 | intron 4 + 47485 | acaatgactgttggagccct C/T gagcaggctgtgtcacgtgg | 5505 | | |
| PEMT | 71 | intron 4 + 48131 | actgggggatcctgaatccc G/A cctcctgatgccagtggagc | 5506 | | |
| PEMT | 72 | intron 4 + 48558 | cacagtgtgaactgttaggc C/G acagccacatcttgccggag | 5507 | | |
| PEMT | 73 | intron 4 + 48702 | gagatgggggcggttcggga G/A gcaaaagcaggaaggcagaa | 5508 | | |
| PEMT | 74 | intron 4 + 50302 | gcatgtgcatgggcagaggc T/C gttcccatctgagtgggacc | 5509 | | |
| PEMT | 75 | intron 4 + 54102 | ggccgcgtgctcctgcagcc A/T tgggctcctctggcagttct | 5510 | | |
| PEMT | 76 | intron 4 + 54220 | cccagggacagatcttctcc G/A ccagacgtctctttctgcct | 5511 | | |
| PEMT | 77 | intron 4 + 54371 | gcagataatgtgcagctggg G/A tgcatgtggttgttgctccc | 5512 | | |
| PEMT | 78 | exon 5 + 79 | tggcctgctactctctaagc G/C tcaccatcctgctcctgaac | 5513 | | |
| PEMT | 79 | intron 5 − 6796 | ggaggaagtcagcttcttac A/C gatggtggctcccagctttc | 5514 | | |
| PEMT | 80 | intron 5 − 6636 | ttttctcctctcaccttttg T/C gttcagaggcagaggtgtgc | 5515 | | |
| PEMT | 81 | intron 5 − 6448 | gttgggccaggctctgacag G/A accctcgggaccagctcctg | 5516 | | |
| PEMT | 82 | intron 5 − 5218 | ggagccctggctgaagaagc C/G ttacgaccaaggcctggagg | 5517 | | |
| PEMT | 83 | intron 5 − 4824 | ggacaggccgggggttgagc G/A gctgcatgaaggagggaggg | 5518 | | |
| PEMT | 84 | intron 5 − 4249 | tcaccagagtgatttcctcg C/A ggcaggtgcctggggtagcc | 5519 | | |
| PEMT | 85 | intron 5 − 4230 | gaggcaggtgcctggggtag C/T cactgggcggggtccatgag | 5520 | | |
| PEMT | 86 | intron 5 − 4182 | ggagagtaaggggtgggggg G/A cacttaggacagggaagctg | 5521 | | |
| PEMT | 87 | intron 5 − 3369 | ccaggtggggccgtgtgcct G/C tggcctggtgtgtggcccag | 5522 | | |
| PEMT | 88 | intron 5 − 2625 | cagggaagctgggccctgaa C/T gagctgggcttttgggccac | 5523 | | |
| PEMT | 89 | intron 5 − 1200 | attattgtgagcatgggaag A/T gcacatttggtcacacatgt | 5524 | | |
| PEMT | 90 | intron 6 + 606 | gcctggctagacgcccacca A/G tgaccctgatgatggcagca | 5525 | | |
| PEMT | 91 | intron 6 + 1229 | tttggtccaggaagggggac G/A gcagccaggagcgtctggat | 5526 | | |
| PEMT | 92 | intron 7 + 716 | atggagatgtgctcccccgg C/G gggtcagaggacctgcggtc | 5527 | | |
| PEMT | 93 | intron 7 + 1537 | ctctgggggacgcataagcc G/A cctccagaggacatcagcca | 5528 | | |
| PEMT | 94 | intron 7 + 1718 | gggcttccaggtgtctgagc T/C ccccggcatgtaggacccca | 5529 | | |
| PEMT | 95 | intron 7 + 2695 | ggctttgggggaccctggac C/T catttctagaaaacagcctt | 5530 | | |
| PEMT | 96 | intron 8 + 140 | ccagggctcccaggtcagag C/T ggccatggtagcttacaatg | 5531 | | |
| PEMT | 97 | 3′flanking + 179 | tacttaggaggcgtcagggg C/T tcacctggccatggccatgg | 5532 | | |
| PEMT | 98 | 3′flanking + 394 | gatgacactgtcattcctaa A/G tgaatggccttgtgctgacc | 5533 | | |
| ALDH1A1 | 1 | intron 1 + 564 | cattatttcttcaaccaagt T/C tgttgccattggaacagata | 5534 | | |
| ALDH1A1 | 2 | intron 1 + 710 | gttctgagagtaactctgaa C/T tttgcctgtttcacactgct | 5535 | | |
| ALDH1A1 | 3 | intron 1 + 3868 | ccctttttatatccagaata C/G aacctaaacttctttctcta | 5536 | | |
| ALDH1A1 | 4 | intron 2 + 2933 | taagtatgctatactatatt T/C gatagatatactatactata | 5537 | | |
| ALDH1A1 | 5 | intron 2 + 1646 | caatgtgattaactgaatac C/T gcaaatatgcactatatatg | 5538 | | |
| ALDH1A1 | 6 | exon 3 + 54 | caggcttttcagattgaatc C/T ccgtggcgtactatggatgc | 5539 | | |
| ALDH1A1 | 7 | intron 3 + 157 | taggccccttaacattgaac T/G attctcaaatagtaatctgc | 5540 | | |
| ALDH1A1 | 8 | intron 3 + 339 | tgagtctcctagaatgatat G/A ttaggtttattcaagcattt | 5541 | | |
| ALDH1A1 | 9 | intron 3 + 655 | agcaattagatgagtcagag C/A ataatatagttgggggaggg | 5542 | | |
| ALDH1A1 | 10 | intron 3 + 735 | gaagccaatttaacataaac C/A aataccaagatcaggtttca | 5543 | | |
| ALDH1A1 | 11 | intron 3 + 863 | acaagtatggttaatcaaag G/A accatttattactcaaatat | 5544 | | |
| ALDH1A1 | 12 | intron 3 + 1757 | agatgacaagatttcttcta T/A ttcaaaaattccctagcaca | 5545 | | |
| ALDH1A1 | 13 | intron 5 + 90 | ttctctaaaacagatagatg C/A ttatgtatttgttaaatgtg | 5546 | | |
| ALDH1A1 | 14 | intron 6 + 213 | caggaagccaaacacaaaag T/C ttggtgtcaaacagtcaact | 5547 | | |
| ALDH1A1 | 15 | intron 6 + 1323 | ttttgaattaaattcttata C/T tgtaacttttaaacttttta | 5548 | | |
| ALDH1A1 | 16 | intron 7 + 638 | gcaaaagaaagtggtggaag C/A atactgtaccatgcaaaaaa | 5549 | | |
| ALDH1A1 | 17 | intron 9 + (1462-1463) | aatggaattctatgtttttt (T) gttgtgattatttatctatc | 5550 | | |
| ALDH1A1 | 17 | intron 9 + (1462-1463) | aatggaattctatgtttttt gttgtgattatttatctatc | 5551 | | |
| ALDH1A1 | 18 | intron 9 + 1757 | tgatctagaatttagtttct A/G taaatgaatagaatccagtg | 5552 | | |
| ALDH1A1 | 19 | intron 12 − 1383 | aatcccacttattactctcc T/G gagagcttcaagtacctata | 5553 | | |
| ALDH1A1 | 20 | 3′flanking + 40 | ttttaagtacaagttttggt T/C acagtgatttcttcttgtca | 5554 | | |
| ALDH1A2 | 1 | 5′flanking − 716 | cagggatcctcattctgagc C/G cgaggcgagggggactcgca | 5555 | | |
| ALDH1A2 | 2 | intron 1 + 314 | cggtcccgactgccacgggg G/Δ aaggcgtcggaaccacttag | 5556 | | |
| ALDH1A2 | 3 | intron 1 + (664-675) | ttttgaactgaagaacttac (T)11-13 ataacgaacattgacatctt | 5557 | | |
| ALDH1A2 | 4 | intron 1 + 1370 | gcatgcagcttagaagtttt A/G ttttatgagggtctctaacc | 5558 | | |
| ALDH1A2 | 5 | intron 1 + 1557 | ggtacgtttttcagaattta A/Δ tttggaagctcttccaattc | 5559 | | |
| ALDH1A2 | 6 | intron 1 + 1934 | tcagctctttagtgagactt C/G taaattttctaagacaagca | 5560 | | |
| ALDH1A2 | 7 | intron 1 + (1971-1980) | agcataatagacaagcagta (T)9-11 aaacgtgaagagcaaaagct | 5561 | | |
| ALDH1A2 | 8 | intron 1 + 2295 | tactgtaagacaatatgtta T/C tgttttttgtcttgctaaac | 5562 | | |
| ALDH1A2 | 9 | intron 1 + 2387 | ttgggacccacatagagtca C/T tacttaaaataaatgaccag | 5563 | | |
| ALDH1A2 | 10 | intron 1 + 2841 | aggaatgtgctttttaaaac T/Δ agatggtgttagtcaaggag | 5564 | | |
| ALDH1A2 | 11 | intron 1 + 3035 | gacttttataattttgtata A/G ctgatattataggaatacac | 5565 | | |
| ALDH1A2 | 12 | intron 1 + 3319 | aaagagttatgttttttttt T/Δ ctgcatctgatattatatgg | 5566 | | |
| ALDH1A2 | 13 | intron 1 + 3474 | ttgtctttttatttattcat T/C taaacttctgttttctgagg | 5567 | | |
| ALDH1A2 | 14 | intron 1 + 4186 | ccttccaaacctttacttaa G/C attgtctgttttagtcataa | 5568 | | |
| ALDH1A2 | 15 | intron 1 + 4222 | cataacttgtcagtcaaact A/G catgttaatagagaacttca | 5569 | | |
| ALDH1A2 | 16 | intron 1 + 4254 | aggacttcaggttttttttt T/Δ aaatactttttcataactat | 5570 | | |
| ALDH1A2 | 17 | intron 1 + 4397 | cccttccactacatgggcct A/G tgttaccatgtgaaattatc | 5571 | | |
| ALDH1A2 | 18 | intron 1 + 5935 | aactccaggttgcaaataga T/C gtttctggtattttaagtag | 5572 | | |
| ALDH1A2 | 19 | intron 1 + 6206 | ttttgaaagcoctcctagca T/G ttctttaatttctttattga | 5573 | | |
| ALDH1A2 | 20 | intron 1 + 9559 | agataaattgatgaattatt C/T actctgtgctgctgatagat | 5574 | | |
| ALDH1A2 | 21 | intron 1 + (9631-9632) | taaaaagaatttctaaaaga (AAGA) ccttttttttgaataactct | 5575 | | |
| ALDH1A2 | 21 | intron 1 + (9631-9632) | taaaaagaatttctaaaaga ccttttttttgaataactct | 5576 | | |
| ALDH1A2 | 22 | intron 1 + 12731 | ctgaaatagaaacctttcag T/A gtaccttgcagagcagtgaa | 5577 | | |
| ALDH1A2 | 23 | intron 1 + 13442 | cagtgtcataaagatccaac G/A gaaatcaaaatatttcatat | 5578 | | |
| ALDH1A2 | 24 | intron 1 + (14173-14176) | tctaaaaaaataaataaata AAAA/Δ gagaaaattaagtttaagat | 5579 | | |
| ALDH1A2 | 25 | intron 1 + 14586 | actcatttattggttcaaag C/G cttcttcaaccttaaaatat | 5580 | | |
| ALDH1A2 | 26 | intron 1 + 14595 | ttggttcaaagccttcttca A/G ccttaggatatgcattgagg | 5581 | | |
| ALDH1A2 | 27 | intron 1 + 14711 | gtttgagacattaacttcta A/G ttcaactaaagatgctagtt | 5582 | | |
| ALDH1A2 | 28 | intron 1 + (15327-15337) | aaagagcacagtagaaagac (T)9-11 aaccctagcaatactattga | 5583 | | |
| ALDH1A2 | 29 | intron 1 + 17258 | atcagtacaatgtgttgggc A/G tacaacacttaatttaaaat | 5584 | | |
| ALDH1A2 | 30 | intron 1 + 18277 | taatacaaatcatttgaagc A/G tttactattaaaaaaacaaa | 5585 | | |
| ALDH1A2 | 31 | intron 1 + 18734 | ctttgagcacctactgcatt T/A taagtgctgttaagatgtgg | 5586 | | |
| ALDH1A2 | 32 | intron 1 + 19081 | ttaatcacctcaatctttaa C/T gaatttcttgatttttcttt | 5587 | | |
| ALDH1A2 | 33 | intron 1 + 21514 | aatcaggatatggggggttc G/A ttctttattctgccacaaat | 5588 | | |
| ALDH1A2 | 34 | intron 1 + 21732 | cattttsaaatagtgcttta A/G taggacttggctgttaaagt | 5589 | | |
| ALDH1A2 | 35 | intron 1 + 21865 | tggcataggtttaaaaatgt C/T tgttgtaggactcttttcca | 5590 | | |
| ALDH1A2 | 36 | intron 1 + 26282 | taaagaaggagaaaaaaaaa A/Δ ctaatctgagactttgcagg | 5591 | | |
| ALDH1A2 | 37 | intron 1 + 27805 | ggatgatgctaoccaaggaa T/C tgcacacttccagacagtac | 5592 | | |
| ALDH1A2 | 38 | intron 1 + 28204 | tcactccattttttaactgt C/G cttcctaaatgtgtggttaa | 5593 | | |
| ALDH1A2 | 39 | intron 1 + 28521 | tctttgttacacttcttaaa T/C cggggtatcagataatcttc | 5594 | | |
| ALDH1A2 | 40 | intron 1 + 49478 | gaataaaaggatagggacat G/T ggtaagaccactttttccct | 5595 | | |
| ALDH1A2 | 41 | intron 1 + 49834 | gcctctcaattttctcatgt G/T taatagagagaaaaccctgc | 5596 | | |
| ALDH1A2 | 42 | intron 1 + 50351 | gactgactggttcataagtt C/G agaaatttcactgtggtcct | 5597 | | |
| ALDH1A2 | 43 | intron 1 + 51181 | tgttattaccatagtagttc C/T gtaacacttggcccttgact | 5598 | | |
| ALDH1A2 | 44 | intron 3 + 654 | ttaacctctcttgagtaaaa G/A gaatccttcagaaccagagg | 5599 | | |
| ALDH1A2 | 45 | intron 3 + 668 | gtaaaaggaatccttcagaa C/T cagaggggatggtacggacc | 5600 | | |
| ALDH1A2 | 46 | intron 3 + 712 | catacacttctgctccgttt G/T ccctgtcattctgtgagcca | 5601 | | |
| ALDH1A2 | 47 | intron 3 + 1273 | tattcatactgtgaaaaagg T/A gtttcatggtgaagaaattc | 5602 | | |
| ALDH1A2 | 48 | intron 3 + 1743 | ccacacctaaatgagattcc C/T gttttaaacactctcaacct | 5603 | | |
| ALDH1A2 | 49 | intron 3 + 2891 | tgcacatatatactcattgt A/G gtttttactaggaactacac | 5604 | | |
| ALDH1A2 | 50 | intron 3 + 2919 | ctaggaactagaccaaactg G/A cagtactagaaatcttttta | 5605 | | |
| ALDH1A2 | 51 | intron 3 + 3054 | tggaaagttctggggactta G/C tatctctccatttctcttcc | 5606 | | |
| ALDH1A2 | 52 | intron 4 + 290 | cattgtgctagattaggtgc T/C ggggtaggtatgaaggggca | 5607 | | |
| ALDH1A2 | 53 | intron 4 + 388 | ctccttgccctcctgaaaca T/C ataagatctactctttggaa | 5608 | | |
| ALDH1A2 | 54 | intron 4 + 461 | gattatggctgattttcagt G/T tctttttaatatttttctct | 5609 | | |
| ALDH1A2 | 55 | intron 4 + 506 | tctatatttctcgaacggcc G/A tgaattactttcataatcta | 5610 | | |
| ALDH1A2 | 56 | intron 4 + 1952 | ttggtccccactccacctgt C/G atttcattattaaaacaaca | 5611 | | |
| ALDH1A2 | 57 | intron 4 + 2079 | ctctatttggcctaacggta C/T cttggttttcttttacttcc | 5612 | | |
| ALDH1A2 | 58 | intron 4 + 2519 | ttgggtcataagagctctct C/G catggtgtctcanacagatg | 5613 | | |
| ALDH1A2 | 59 | intron 4 + (2840-2851) | tttgtctctgcatacttggc (T)11-13 cacagtgaagtctggaatat | 5614 | | |
| ALDH1A2 | 60 | intron 4 + 7231 | aataggatacaaatacacaa A/T gatagtgattcagatcctaa | 5615 | | |
| ALDH1A2 | 61 | intron 4 + 7958 | taaaatcgtttttattgtta C/T taggtatataaaatttgcta | 5616 | | |
| ALDH1A2 | 62 | intron 4 + 8090 | tctgattttatcactgttta C/T agattgcttagtcatactca | 5617 | | |
| ALDH1A2 | 63 | intron 4 + 12823 | tgttagcctgtagctaaatg C/T ttttcaaatatgtgaacggt | 5618 | | |
| ALDH1A2 | 64 | intron 4 + 12939 | atgaggtccgacttttaaga T/C ttttgtctacattttcttcc | 5619 | | |
| ALDH1A2 | 65 | intron 4 + 14935 | tattgatggagttcttttta T/G aaatggacttttaccttctt | 5620 | | |
| ALDH1A2 | 66 | intron 4 + 15321 | gcatttgtgtgtctgagaga C/T atatccagaaatatgctatg | 5621 | | |
| ALDH1A2 | 67 | intron 4 + 15412 | tttcaagtttatttctgttt T/G tttttttttttttttttttg | 5622 | | |
| ALDH1A2 | 68 | intron 5 + 1888 | aatccaaacatctgtacttt G/T tagtggacaagatttatgtc | 5623 | | |
| ALDH1A2 | 69 | intron 7 + 9166 | gaaaagctactttattcaaa G/A ataaaagtattttaagaaaa | 5624 | | |
| ALDH1A2 | 70 | intron 7 + 9914 | aagctggagaaaatactagg C/T tttcctcaacagtgatttcc | 5625 | | |
| ALDH1A2 | 71 | intron 7 + 18942 | tttggaggggaactaatccc G/A tgacttctaggttatctctt | 5626 | | |
| ALDH1A2 | 72 | intron 7 + 19820 | ttcacccctcattttaggtt A/G ggggaggtggcttgctacag | 5627 | | |
| ALDH1A2 | 73 | intron 7 + 19826 | cctcattttaggttagggga G/A gtgtcttgctacagttttag | 5628 | | |
| ALDH1A2 | 74 | intron 7 + 19913 | cgtgaatcattcagtatttt A/G tttaaaaataccagtttgaa | 5629 | | |
| ALDH1A2 | 75 | intron 7 + (20110-20111) | catgatttattctctaacta (ACTA) tgctaagtcaaagattctgc | 5630 | | |
| ALDH1A2 | 75 | intron 7 + (20110-20111) | catgatttattctctaacta tgctaagtcaaagattctgc | 5631 | | |
| ALDH1A2 | 76 | intron 7 + 21857 | acaatgaaaattaagaaagg A/T gaagagggaagaagnagaga | 5632 | | |
| ALDH1A2 | 77 | intron 7 + 21929 | tacaagacacaggcatcttt A/G actagtttactgggatctct | 5633 | | |
| ALDH1A2 | 78 | intron 7 + 23308 | ggctttcacttcggaaacct G/T tgggttataacaaagtactg | 5634 | | |
| ALDH1A2 | 79 | intron 7 + 23554 | gacattggtgaaaaccaggg C/T tgtttaggagtgtcctgtcc | 5635 | | |
| ALDH1A2 | 80 | intron 7 + (23701-23703) | catctgagatttgccttgtg GTG/Δ tttaccgagttagtgggtgc | 5636 | | |
| ALDH1A2 | 81 | intron 7 + 26479 | gatacatgaacaatttgttt T/C atcctcatgatatctttcaa | 5637 | | |
| ALDH1A2 | 82 | intron 7 + 26561 | taaaggccacaatgcagtga T/C tgaaatctccagttacattt | 5638 | | |
| ALDH1A2 | 83 | intron 7 + 26662 | tttccttagtccttccatca C/T gaaactaaagctgtcttcca | 5639 | | |
| ALDH1A2 | 84 | intron 8 + 76 | tttatatctccacttttgat G/A ggacactagcaaaagatatt | 5640 | | |
| ALDH1A2 | 85 | intron 8 + (700-711) | accatctcattcagtgattc (T)11-12 ccctccacttgttgccaggc | 5641 | | |
| ALDH1A2 | 86 | intron 8 + 724 | ttttttttccctccacttgt T/C gccaggcagagctgctttcc | 5642 | | |
| ALDH1A2 | 87 | intron 8 + 800 | cagattgcttgaatttcagc C/A ccagcttggaatttgcagag | 5643 | | |
| ALDH1A2 | 88 | intron 8 + 1251 | gatttctgtgaaaattgaga G/A gatctggcaacctggggctc | 5644 | | |
| ALDH1A2 | 89 | intron 8 + 1627 | ggcccctccccaggcaaagc G/A gtgagaacatggctgtttcc | 5645 | | |
| ALDH1A2 | 90 | exon 9 + 141 | tggagcgggccaagaggcgc G/A tagtggggagtccctttgac | 5646 | | |
| ALDH1A2 | 91 | intron 9 + 778 | aaccagtctggacagatccc T/C tgtagcttgtgaaagtgtag | 5647 | | |
| ALDH1A2 | 92 | intron 9 + 801 | tagcttgtgaaagtgtagga A/G gtgaagggctggctcacttc | 5648 | | |
| ALDH1A2 | 93 | intron 9 + 868 | tctgaaggcctcgtgtactt T/C agtggggtggggagggccac | 5649 | | |
| ALDH1A2 | 94 | intron 9 + 1338 | aatttttgcctctttttact A/G tcaatacaacttgctaagtt | 5650 | | |
| ALDH1A2 | 95 | intron 10 + (227-229) | ctatgtgcttatgattatta TTA/Δ gccaacagaacaatcagaat | 5651 | | |
| ALDH1A2 | 96 | intron 10 + 316 | ctaaatgtcggtcactggga T/C gttaaccaggatagagaatc | 5652 | | |
| ALDH1A2 | 97 | intron 10 + 368 | ctttacatctctgcaagaga G/A ggacaaggagcaaatcagcc | 5653 | | |
| ALDH1A2 | 98 | intron 10 + 660 | gtaaacttgcattgaaatgt G/A gaaagcaggtaaaggaatca | 5654 | | |
| ALDH1A2 | 99 | intron 11 + 104 | tggggaataccaaaagcaac C/T aaagttcaccagaaaagggg | 5655 | | |
| ALDH1A2 | 100 | intron 11 + 229 | aaacttctaaaagaaatacc A/G tgccagtcagatiatgtgct | 5656 | | |
| ALDH1A2 | 101 | intron 12 + 117 | catacattcaacaaacattt C/T gtggagcacatgctactata | 5657 | | |
| ALDH1A2 | 102 | intron 12 + 691 | gatagggaagatcactgtga A/G ctggaaaaatctcggaaacc | 5658 | | |
| ALDH1A2 | 103 | intron 12 + 1934 | catcttgtctagattgcatg T/C ttgtttgtttgtttgtctct | 5659 | | |
| ALDH1A2 | 104 | intron 12 + 1973 | ctacttacccccaaaacatg T/A tttctctttcttaaatgacc | 5660 | | |
| ALDH1A2 | 105 | intron 12 + 2722 | ccagagtgactccagtatac C/A tcactgcccaggacccacag | 5661 | | |
| ALDH1A2 | 106 | intron 12 + 3855 | cacttgaaagcaaccataat T/C gtgaggtttctgatgctgtg | 5662 | | |
| ALDH1A2 | 107 | intron 12 + 4185 | ttgctttaagcgaaatgaac T/C atacggacaggagaacaggc | 5663 | | |
| ALDH1A2 | 108 | intron 12 + 4991 | acaggaacacttagacatgc A/G acccactcccaccctccgtc | 5664 | | |
| ALDH1A2 | 109 | intron 12 + (5018-5019) | cccaccctccgtcttggggg (G) aggaaagcacactactgtcc | 5665 | | |
| ALDH1A2 | 109 | intron 12 + (5018-5019) | cccaccctccgtcttggggg aggaaagcacactactgtcc | 5666 | | |
| ALDH1A2 | 110 | intron 12 + (5051-5052) | actgtcccaaagaactaata (A) ctgaaccagtgctgccttgt | 5667 | | |
| ALDH1A2 | 110 | intron 12 + (5051-5052) | actgtcccaaagaactaata ctgaaccagtgctgccttgt | 5668 | | |
| ALDH1A2 | 111 | intron 12 + (5300-5302) | ttaaagttttaaaaaaactt CCT/Δ taaaaactactcatgagatg | 5669 | | |
| ALDH1A2 | 112 | intron 12 + 5405 | catcccaggacttgctgttc G/C caggtgataaactgcacctc | 5670 | | |
| ALDH1A2 | 113 | intron 12 + 5435 | aactgcacctccccaggact C/A ccgctgcactcacatgcagc | 5671 | | |
| ALDH1A2 | 114 | 3′flanking + 449 | tttgggccgggaacaatttt T/C caaggttgtaaagccaaatt | 5672 | | |
| ALDH1A2 | 115 | 3′flanking + 597 | acctgggatattcctgaccc A/C atctggttttcttttaccca | 5673 | | |
| ALDH1A2 | 116 | 3′flanking + 669 | atagagactggaagtcatca T/C gtgcagttcaccgcttctga | 5674 | | |
| ALDH1A2 | 117 | 3′flanking + 1122 | cgtgctccactgagctcctc T/G gtcacaccccattcttgccc | 5675 | | |
| ALDH1A2 | 118 | 3′flanking + 2214 | tgcagctgtaaaaagaaatc T/C gtaaatggtgaccgtactac | 5676 | | |
| ALDH1A3 | 1 | 5′flanking − 1425 | cagtgttagccagccgatat C/T ggtcaaggctgccccgctcg | 5677 | | |
| ALDH1A3 | 2 | 5′flanking − 1379 | ccattatcccctttccccgg C/T ctcagctgtgcactccaggc | 5678 | | |
| ALDH1A3 | 3 | 5′flanking − 1270 | aacttacccctctatccagc T/A ctatccagaaggacaccagg | 5679 | | |
| ALDH1A3 | 4 | 5′flanking − (1214-1213) | acggaggcctcaaaacagga (GGA) aaataagcagacccctcccc | 5680 | | |
| ALDH1A3 | 4 | 5′flanking − (1214-1213) | acggacgcctcaaaacagga aaataaggagacccctcccc | 5681 | | |
| ALDH1A3 | 5 | 5′flacking − 1103 | gcacagcttttgtcaggagt C/T cgtgcctccggtctttgttc | 5682 | | |
| ALDH1A3 | 6 | intron 1 + 986 | gccttaactttccccacctt T/G ggcttctcttgatttttgct | 5683 | | |
| ALDH1A3 | 7 | intron 1 + 1462 | gtacaggatttcaaaatact G/A tatatagaaaccagacagta | 5684 | | |
| ALDH1A3 | 8 | intron 1 + 1661 | cctgttgtcttggtcggtgc G/A cascctttgccagttaaagg | 5685 | | |
| ALDH1A3 | 9 | intron 1 + 2360 | agaggatagaagtcccttct A/G atttagagggcctctttctt | 5686 | | |
| ALDH1A3 | 10 | intron 1 + 2516 | tgaaaacatattctttttga G/A tttagctgagtggcctgttg | 5687 | | |
| ALDH1A3 | 11 | intron 1 + 2624 | cctgagacaccttacagctc C/T gtcctgcttccatgtcattc | 5688 | | |
| ALDH1A3 | 12 | intron 1 + 3255 | tttcatctttctacaaatgg G/C cccctcttcctggctgcact | 5689 | | |
| ALDH1A3 | 13 | intron 1 + (3643-3656) | gcttcagaggtttttgtggg (T)12-14 aacattctatcaacttttaa | 5690 | | |
| ALDH1A3 | 14 | intron 1 + 4265 | ccaaaagccctctcttttaa T/C atgacattaataagacaatt | 5691 | | |
| ALDH1A3 | 15 | intron 1 + 5187 | caagatggataagacgtcac C/T taaggtccttagcatgttga | 5692 | | |
| ALDH1A3 | 16 | intron 2 + 43 | ctctaagtaattcaattatg G/T atgaccaaaggataacgaaa | 5693 | | |
| ALDH1A3 | 17 | intron 2 + 127 | cagggcctgggctagctgcg T/C gaattggcatgtggttctca | 5694 | | |
| ALDH1A3 | 18 | intron 2 + (285-300) | aggaaaggttttcttttttc (T)16-17 atcaattatttggacctgga | 5695 | | |
| ALDH1A3 | 19 | intron 2 + 770 | cgtgtgcagagtaggcttgg A/G ttttatcttgcccatgagtt | 5696 | | |
| ALDH1A3 | 20 | intron 2 + 1216 | actcggtagagtcactcctg A/C ctggtgtcccacatccactc | 5697 | | |
| ALDH1A3 | 21 | intron 3 + 81 | accatggggtatgggaaaaa A/C gatcacggtcctggttttct | 5698 | | |
| ALDH1A3 | 22 | intron 3 + 236 | gctcagcttcttgaccaact T/G gttgtctataggcagttgag | 5699 | | |
| ALDH1A3 | 23 | intron 3 + 1467 | ggcccggttgtaggggagga G/T atctcctttctggcctttgc | 5700 | | |
| ALDH1A3 | 24 | intron 3 + 1725 | ccacatgttccccgggtgag A/G gtagctccctcccaggctaa | 5701 | | |
| ALDH1A3 | 25 | intron 3 + 3777 | gccagaagtagatcccccca A/G ttcagctgctgcattactcg | 5702 | | |
| ALDH1A3 | 26 | intron 3 + 3829 | caagtcactgggcccttagc G/C tccgtgcctgcaccttgaag | 5703 | | |
| ALDH1A3 | 27 | intron 3 + 4299 | tcactttccacagccacact G/A gccagcctccccgagaagga | 5704 | | |
| ALDH1A3 | 28 | intron 4 + 84 | agagccccccctgactgttt C/G cctaaggcaccattcccaac | 5705 | | |
| ALDH1A3 | 29 | intron 4 + 126 | ccactccctctccaaatggt A/G ctgccaattcttcttctaag | 5706 | | |
| ALDH1A3 | 30 | intron 6 + (290-291) | tagagaattttcaggggggg (G) tcaaccaagagggagccaaa | 5707 | | |
| ALDH1A3 | 30 | intron 6 + (290-291) | tagagaattttcacgccggg tcaaccaagagggagccaaa | 5708 | | |
| ALDH1A3 | 31 | intron 6 + 705 | aacagctggtgatgagccaa T/G tttccactttcctttgctga | 5709 | | |
| ALDH1A3 | 32 | intron 7 + 56 | ggggcgtgttatttcacacc C/T gtcagcttttcctttgacag | 5710 | | |
| ALDH1A3 | 33 | intron 7 + 1107 | gatgctgttactctccttcc A/G gacagacactgccctgtgga | 5711 | | |
| ALDH1A3 | 34 | intron 7 + 1610 | aagagccacacagaaccacc C/G ccctactgggctgttggaat | 5712 | | |
| ALDH1A3 | 35 | intron 7 + 1820 | cacctgtaagtggagcggct T/C agaccaaggatcccaggatc | 5713 | | |
| ALDH1A3 | 36 | intron 8 + 963 | gagaaaggacaggaggagga C/T acaggctctcaggaaggaaa | 5714 | | |
| ALDH1A3 | 37 | intron 8 + 1824 | accattcttatccactaagc G/A tgtcccccaagatcttattc | 5715 | | |
| ALDH1A3 | 38 | intron 8 + 2384 | cgcctccctcgccccctccc C/A tccagtggacttggcagtgg | 5716 | | |
| ALDH1A3 | 39 | intron 9 + 24 | atccccctggtgtgtgtgaa A/C ccatggtgcttgtctagggg | 5717 | | |
| ALDH1A3 | 40 | intron 9 + 91 | gcctacagggtccctctccg T/C gaaaggaatgctgacctgtc | 5718 | | |
| ALDH1A3 | 41 | intron 9 + 219 | actgaggcatgggaggaggg C/G gctattcccagggcagaagg | 5719 | | |
| ALDH1A3 | 42 | intron 9 + 435 | ccagacggagagagcctggg G/A caggagaatgtatctccacc | 5720 | | |
| ALDH1A3 | 43 | intron 9 + 1472 | ttcacttttgaggccagata C/T accgatttcttccaagagaa | 5721 | | |
| ALDH1A3 | 44 | intron 9 + 2038 | taaacaatgtgttcctacgg G/A ctctccagggagtgtggact | 5722 | | |
| ALDH1A3 | 45 | intron 9 + 2124 | caaacagggtctgccagatg G/A catatgcccagcagccaggg | 5723 | | |
| ALDH1A3 | 46 | intron 9 + 2154 | agcagccagggaggacgtgc G/C gttgggcgaagcccctgtgt | 5724 | | |
| ALDH1A3 | 47 | intron 9 + 2197 | cttttggcccctcagggagg G/A gaagagcagctcagcagcat | 5725 | | |
| ALDH1A3 | 48 | intron 9 + 2466 | ttcttagttcctcatgtttc C/T ctctagaatgttttcgtgtg | 5726 | | |
| ALDH1A3 | 49 | intron 9 + 3655 | gattggtcaagtggcatgca C/T ggtttatgccctctctcctg | 5727 | | |
| ALDH1A3 | 50 | intron 9 + 3954 | gggtgcgcttttgacaactg C/G tcagtagcgtgttcacaagc | 5728 | | |
| ALDH1A3 | 51 | exon 10 + 88 | tggaatgcgggggctcagcc A/C tggaagacaagsggctcttc | 5729 | | |
| ALDH1A3 | 52 | intron 18 + 8 | tgccaaagaggaggtacaag G/A gggctgtggcaaggctacga | 5730 | | |
| ALDH1A3 | 53 | intron 10 + 307 | ctctctgattttctaacaca A/C ccggtccccgagtcagtcat | 5731 | | |
| ALDH1A3 | 54 | intron 10 + 378 | gtgggttttgccaggaatca G/A ttcaagaacctgtggattca | 5732 | | |
| ALDH1A3 | 55 | intron 10 + 975 | aatattgtgtcattccttcc C/G ctggtagttattatggaaac | 5733 | | |
| ALDH1A3 | 56 | intron 10 + 1088 | cagtgccaggagccaggggg C/T cttctccagatgaccttgag | 5734 | | |
| ALDH1A3 | 57 | intron 11 + 105 | ttgtctacattgtatattat A/G taccaagccctgtctcagtg | 5735 | | |
| ALDH1A3 | 55 | intron 11 + 274 | agggctccagtacctgtgcc T/G gtggcccctgtgctgtactg | 5736 | | |
| ALDH1A3 | 59 | intron 11 + 1088 | cagtgccaggagccaggggg T/A cttctccagatgaccttgag | 5737 | | |
| ALDH1A3 | 60 | intron 12 + 96 | ctccaatgtgctgagacccc G/A tcccccccacaccgccgctc | 5738 | | |
| ALDH1A3 | 61 | intron 12 + 1537 | gggccttggttggggccttt G/T tgtggctctcttttgagatt | 5739 | | |
| ALDH1A3 | 62 | intron 12 + 1660 | gtccccctcccacctcagtc C/Δ10 tgctttgtagtccatccgtg | 5740 | | |
| ALDH1A3 | 63 | intron 12 + 5642 | tctgtgctaacgtctgcttc T/C ctcatgccccctaggctggc | 5741 | | |
| ALDH1A3 | 64 | exon 13 + 104 | gggctccttcctcaaacatc G/C gacggcggaatgtggcagat | 5742 | | |
| ALDH1A3 | 65 | exon 13 + 281 | ataggttgtctgtgaaatcg C/T agtcctgcctggggagggag | 5743 | | |
| ALDH1A3 | 66 | 3′flanking + 743 | gtgagcaggaaactgtagga G/A aaggatattttccctcattt | 5744 | | |
| ALDH1A3 | 67 | 3′flanking + 1145 | gcctcccagctaccccaccc A/G cctcaggaggggtcattcca | 5745 | | |
| ALDH1A3 | 68 | 3′flanking + 1185 | aacctagggtgctgagaatc T/C gggtgggattaccagcaaaa | 5746 | | |
| ALDH1A3 | 69 | 3′flanking + 1600 | acaccacgccctgcaaattg T/C tgggaacttgtcggtggcaa | 5747 | | |
| ALDH1A3 | 70 | 3′flanking + 1847 | caggagccctgcggctgcgg C/G ggttctgtgaaatggcagtg | 5748 | | |
| ALDH1B1 | 1 | intron 1 + 134 | cgttgcactgtaggactcct C/T ccacgtcccctaatcccatc | 5749 | | |
| ALDH1B1 | 2 | intron 1 + 367 | gcagttcccgcggatagaga A/G ggtccggtccttcgggctgt | 5750 | | |
| ALDH1B1 | 3 | intron 1 + 405 | tgtgggtgaactgtaaaaaa C/T tgcctgtattcaggaggata | 5751 | | |
| ALDH1B1 | 4 | intron 1 + 2002 | cttcaactaatctgggaaca C/T tacactctgtttaattttca | 5752 | | |
| ALDH1B1 | 5 | intron 1 + 2157 | tgggaaagctgaeaagggat G/T ctgagacctgtggttggggg | 5753 | | |
| ALDH1B1 | 6 | exon 2 + 192 | ccgacggtcaaccctaccac T/C ggggaggtcattgggcacgt | 5754 | | |
| ALDH1B1 | 7 | exon 2 + 265 | cgtgaaagcagcgggggaag C/T cttccgcctggggtccccat | 5755 | | |
| ALDH1B1 | 8 | exon 2 + 329 | gcggggccggctgctgaacc G/T cctggcagacctagtggagc | 5756 | | |
| ALDH1B1 | 9 | exon 2 + 614 | acttgccccggcactcgcca C/T aggcaacactgtggttatga | 5757 | | |
| ALDH1B1 | 10 | 3′flanking + 168 | aaagtgcaactgtaagaccc G/A tagagaaaaactctggttcc | 5758 | | |
| ALDH1L1 | 1 | intron 1 + 252 | cgcagcgccaggagtggccc G/C ccgaggatctggccggccgc | 5759 | | |
| ALDH1L1 | 2 | intron 1 + 544 | ctcaggggctgcgctggagt C/T ccagctccagccactgcgct | 5760 | | |
| ALDH1L1 | 3 | intron 1 − 6596 | cagatttttcttaaggtgca C/G tagccactgaggatattttt | 5761 | | |
| ALDH1L1 | 4 | intron 1 − 6513 | caattatggtttatcttagg G/A acatgtttatagagatagta | 5762 | | |
| ALDH1L1 | 5 | intron 1 − 6478 | atagtattcttacttagctt G/A cattctaaattttgttccct | 5763 | | |
| ALDH1L1 | 6 | intron 3 + 240 | gtggcattagggtcctggag A/G agggctatagagaagcccag | 5764 | | |
| ALDH1L1 | 7 | intron 2 + 1326 | gaggaggagaccggagagga G/C agccagtccagtcagggccc | 5765 | | |
| ALDH1L1 | 8 | intron 3 + 386 | gtcctactctaacttccact G/A ccgctgctctgggcagcaca | 5766 | | |
| ALDH1L1 | 9 | intron 4 + 271 | gggcccgttcaatagacaag G/C aaggctaaaggcagggagtg | 5767 | | |
| ALDH1L1 | 10 | intron 4 + 356 | taggattctatttctctctc C/T ttcactcgttgattctcctt | 5768 | | |
| ALDH1L1 | 11 | intron 4 + 608 | gtgctctgataggctgtctc A/C gtcacatgcttcctgctggg | 5769 | | |
| ALDH1L1 | 12 | intron 4 + 664 | ggtcacatggcctgagcggc A/G gggcggctcagtcacctccc | 5770 | | |
| ALDH1L1 | 13 | intron 4 + 785 | gaggctggcttgcccctgcc C/G gaggacaggctggcagggac | 5771 | | |
| ALDH1L1 | 14 | intron 4 + 874 | ccctggggagcccttgctgt T/G tgggcgcagcaggaagagca | 5772 | | |
| ALDH1L1 | 15 | intron 4 + 1349 | tccctcaggctcttgctcac G/A tgggcccagacctcttggct | 5773 | | |
| ALDH1L1 | 16 | intron 4 + 1799 | gtggggctgggaaggaggca G/A ggtcctattgctggggatag | 5774 | | |
| ALDH1L1 | 17 | intron 4 + 1815 | ggcagggtcctattgctggg G/A atagcaacccactggatctc | 5775 | | |
| ALDH1L1 | 18 | intron 5 + 272 | aaagcccacagggagataag A/G gtgggagttagggggcaaaa | 5776 | | |
| ALDH1L1 | 19 | intron 5 + 301 | tagggggcaaaacgtccgcc G/A tagtgcgagcagtcttcaag | 5777 | | |
| ALDH1L1 | 20 | intron 5 + 343 | caaggtstgagggacagtgc G/A ggtctctggagcaatagcca | 5778 | | |
| ALDH1L1 | 21 | intron 6 + 926 | cctgcctgggctagtggctt C/T gggggcttcttctcacccac | 5779 | | |
| ALDH1L1 | 22 | exon 7 + 41 | aacgctgaacacttcaggcc T/C ggtgcccgagsgagacgctt | 5780 | | |
| ALDH1L1 | 23 | intron 7 + 305 | cctagaatcagagagaagcc C/T tcccagggagcctgggttca | 5781 | | |
| ALDH1L1 | 24 | intron 7 + 837 | gtcnggacaaaccccatggg C/T gtggtacccccagccgtgtt | 5782 | | |
| ALDH1L1 | 25 | intron 7 + 866 | cccagccgtgttgctgtgtc C/T gccctaccagagtgaggcgt | 5783 | | |
| ALDH1L1 | 26 | intron 7 + 884 | tccggcctaccagagtgagg C/T gtggcagtatggggcctggc | 5784 | | |
| ALDH1L1 | 27 | intron 7 + 1118 | aatgttccagaaaatcatgc G/C agccagtaagggcagaggaa | 5785 | | |
| ALDH1L1 | 28 | intron 7 + 1168 | aaagtaaaggttcaggagaa G/A tctagcctggggctgctccc | 5786 | | |
| ALDH1L1 | 29 | intron 7 + 1451 | cagggcacccacagcatgtg T/C ccagagacctgcaaagacag | 5787 | | |
| ALDH1L1 | 30 | intron 7 + 1489 | caggaatgcaaagaaggcaa T/C taagtgtcttaagaggaagc | 5788 | | |
| ALDH1L1 | 31 | intron 7 + 1579 | tcagggtgggaggggagtga G/A gagagaccagctgagcacac | 5789 | | |
| ALDH1L1 | 32 | intron 7 + 1691 | ctggctgggctttagcttgc A/C gaaagctccagaacatcttt | 5790 | | |
| ALDH1L1 | 33 | intron 8 + 1632 | tcaggtttgcatttgttcac T/C gtgcacattcagagttccac | 5791 | | |
| ALDH1L1 | 34 | intron 8 + 1799 | gctcaagtcctcctctagct G/C ttcaccgtgcagccccctaa | 5792 | | |
| ALDH1L1 | 35 | intron 8 + 1986 | ggtggaggggcctggcctgt G/T gctgttcaggagaacgctcc | 5793 | | |
| ALDH1L1 | 36 | intron 8 + 2002 | ctgtggctgttcaggagaac A/G ctccaagagcctgctgtggg | 5794 | | |
| ALDH1L1 | 37 | intron 8 + 2627 | aaagaggagagcggggggtg C/T ttgtgccaggggttggggga | 5795 | | |
| ALDH1L1 | 38 | intron 8 + 2646 | gcttgtgccaggggttgggg G/A aactggttctgattgggcct | 5796 | | |
| ALDH1L1 | 39 | intron 8 + 2925 | ctgctgccctccataggtcc C/G agactgaatccttcagagga | 5797 | | |
| ALDH1L1 | 40 | exon 9 + 4 | caggtcttgctttgcagagt G/T tttggcagcggatcctcccc | 5798 | | |
| ALDH1L1 | 41 | exon 10 + 109 | cagctgttagtgaggaagct G/T cgaggggacgataaggaggg | 5799 | | |
| ALDH1L1 | 42 | intron 10 + (671-672) | tggcattttcctctgtgtga (AG) gtcctcttagcccaccctaa | 5800 | | |
| ALDH1L1 | 42 | intron 10 + (671-672) | tggcattttcctctgtctga gtcctcttagcccaccctaa | 5801 | | |
| ALDH1L1 | 43 | intron 11 + 8 | caccgatggaagtgtgagtg C/A aggcccagcaccccttctcc | 5802 | | |
| ALDH1L1 | 44 | intron 11 + 447 | atgagccaaagcacgcctat G/A gtagatacacacgtgaacat | 5803 | | |
| ALDH1L1 | 45 | intron 11 + 601 | ctcaaaatgagtcatttgag A/G ggagttaatgaaagactcat | 5804 | | |
| ALDH1L1 | 46 | intron 11 + 639 | catctgcaaagggagaggga G/A ggggtagggacacagacagg | 5805 | | |
| ALDH1L1 | 47 | intron 12 + 66 | ctgggcagtggcacggggag G/Δ acttctgtggaggccctttt | 5806 | | |
| ALDH1L1 | 48 | intron 12 + 478 | ctattaaaaaaaaaaaaaaa A/Δ tttaagccagggagaaaggg | 5807 | | |
| ALDH1L1 | 49 | intron 12 + 684 | tcctgggagaagagagggtg C/T ggccagatgagccgagaaca | 5808 | | |
| ALDH1L1 | 50 | intron 12 + 767 | cgtctaggggtaggaagaca A/G gttatggcgtggtcccaacg | 5809 | | |
| ALDH1L1 | 51 | intron 12 + 1014 | tcataggttccagtcccctt C/T gaaagccccctaattctaga | 5810 | | |
| ALDH1L1 | 52 | intron 12 + 1359 | ctggttctgcctcagctcag C/T acagcagaggataggtctag | 5811 | | |
| ALDH1L1 | 53 | intron 12 + 1734 | ggtggtccaggctgctggtg G/T taagtagggcgggcggagcc | 5812 | | |
| ALDH1L1 | 54 | intron 12 + 1901 | ttcagcagcctaagtgaatt G/A acaatagaatagtcctgcaa | 5813 | | |
| ALDH1L1 | 55 | intron 12 − 470 | gggatggggccacatctcca T/C ctctggagatgccaggctca | 5814 | | |
| ALDH1L1 | 56 | intron 12 − 334 | aagggcagcctcttgggcca T/C gacccctttgctgtctgcag | 5815 | | |
| ALDH1L1 | 57 | intron 12 − 325 | atcttgggccatgacaaatt T/C gctgtctgcagcaagtgggt | 5816 | | |
| ALDH1L1 | 58 | intron 12 − 221 | gaaggaagcgagggaagatc G/C aggaaaggagagagggacag | 5817 | | |
| ALDH1L1 | 59 | intron 12 − 4 | cccgcttcccctcaccctgg T/C gcaattggcagatctcatcg | 5818 | | |
| ALDH1L1 | 60 | intron 13 + 34 | tcccaggcagtgtgagcaca T/C gcagactggaccagccatat | 5819 | | |
| ALDH1L1 | 61 | intron 13 + 58 | gaatggcccagccatatagg A/G gaactaaaagggcagcacag | 5820 | | |
| ALDH1L1 | 62 | intron 13 + 125 | ccaaaactggtggcttggaa T/C gaaaaatgtttattagcttg | 5821 | | |
| ALDH1L1 | 63 | intron 13 + 126 | cacaaatggtggattggaat G/A aaacctgtttattagattgt | 5822 | | |
| ALDH1L1 | 64 | intron 13 + 281 | acctgcatccagacgagtta T/G ggtgttgacagagttcagtt | 5823 | | |
| ALDH1L1 | 65 | intron 13 + 299 | tcgggtgttgacagagttca A/G ttccgtgtggatgcagggct | 5824 | | |
| ALDH1L1 | 66 | intron 14 + 121 | catttatcaaacagccatac A/G tgtgcttcttgagcacctgc | 5825 | | |
| ALDH1L1 | 67 | intron 14 + 167 | gccaggcattgttgtaagga C/T ttgaggacaattgtatttaa | 5826 | | |
| ALDH1L1 | 68 | intron 14 + 205 | taatctcccagtaacactgg A/C tcagtaaggtccacggtggg | 5827 | | |
| ALDH1L1 | 69 | intron 14 + 219 | cactggatcagtcaggtcca C/G ggtgggaaacaagagtaaac | 5828 | | |
| ALDH1L1 | 70 | intron 14 + 2275 | tctcatctgtgatgcataag T/C cagaaatctgctcccagcat | 5829 | | |
| ALDH1L1 | 71 | intron 14 + 2431 | agaatgactgagtgatgaga C/G ctagagagccccagcccagg | 5830 | | |
| ALDH1L1 | 72 | intron 14 + 2660 | agccaagcatttcttgggga C/T accaagaaaccttgcttggt | 5831 | | |
| ALDH1L1 | 73 | intron 14 + 2740 | aactccaccctcaccgtccg T/C gcagctccccaggagcgtca | 5832 | | |
| ALDH1L1 | 74 | intron 14 + 2756 | tcaatgaagatccccaggag T/C gtcagagggaagaggagggg | 5833 | | |
| ALDH1L1 | 75 | intron 14 + 2805 | ccgaacagcaggagaatgga T/C ccaagggagggagggaaggg | 5834 | | |
| ALDH1L1 | 76 | intron 14 + (3636-3637) | tctcctgggtgtgtgtgggg (G) tgtggggcagctcccctatc | 5835 | | |
| ALDH1L1 | 76 | intron 14 + (3636-3637) | tctcctgggtgtgtgtgggg tgtggggcagctcccctctc | 5836 | | |
| ALDH1L1 | 77 | intron 14 + 4347 | tacaggacagaaacagcagg C/T gtgagctgactctaagaggg | 5837 | | |
| ALDH1L1 | 78 | intron 15 + 380 | atgtcccttatgtcgcttca A/G agaacagaagtactggagag | 5838 | | |
| ALDH1L1 | 79 | intron 15 + (1055-1056) | gccacaatctgcagctactc (C) tcccagcttgctgctgggct | 5839 | | |
| ALDH1L1 | 79 | intron 15 + (1055-1056) | gccacagtctgaagctacta tcccagcttgctgctgggct | 5840 | | |
| ALDH1L1 | 80 | intron 17 + 15 | gaaaaggtgagtggctgggg G/C tggagcagaggaggggctgc | 5841 | | |
| ALDH1L1 | 81 | intron 17 + 44 | aggaggggctgctgtgagtg C/T gcctgggacatggcagtgat | 5842 | | |
| ALDH1L1 | 82 | intron 17 + 51 | gctgctgtgagtgcgcctgg G/A acatggcagtgctgtccaca | 5843 | | |
| ALDH1L1 | 83 | intron 17 − (2224-2223) | atggtgtaatctcccagaat CT/Δ gtaaataaaaaaaaatatga | 5844 | | |
| ALDH1L1 | 84 | intron 19 + 140 | agcgtcatcacaagcatagc G/A tggcaggcagcaggcttagg | 5845 | | |
| ALDH1L1 | 85 | intron 19 + (51-52) | tggttcactgggacagcagc GC/Δ ctggctggagggggttggag | 5846 | | |
| ALDH1L1 | 86 | intron 19 + 399 | tcaggtcagcctgggcctga C/A catggacaggggccctggag | 5847 | | |
| ALDH1L1 | 87 | intron 19 + 608 | caaccagatttatccaatca A/G ccacacctggaagagcaggc | 5848 | | |
| ALDH1L1 | 88 | intron 19 + (669-670) | atgggccatcctgagtccgc (C) ttgggaggtttgtaatccct | 5849 | | |
| ALDH1L1 | 88 | intron 19 + (669-670) | atgggccataatgagtccac ttgggaggtttgtaatgcct | 5850 | | |
| ALDH1L1 | 89 | intron 19 + 1794 | gtcctgtctgggggtcttaa G/C ggagtaatgagaattccaca | 5851 | | |
| ALDH1L1 | 90 | intron 19 + 1969 | tgatcggggtgcggtttggg G/T cgacaggacaggagaagaga | 5852 | | |
| ALDH1L1 | 91 | intron 19 + 1972 | tcggggtgcggtttggggcg A/G caggacaggagaagagaata | 5853 | | |
| ALDH1L1 | 92 | intron 19 + 2083 | tgagaagagcagaggggtgt G/T ccgggtgctcgagtcacacc | 5854 | | |
| ALDH1L1 | 93 | intron 19 + 2119 | acacctgtgtctgattaggg C/T tgattaggggtgcagagttt | 5855 | | |
| ALDH1L1 | 94 | intron 20 + 1388 | ttaccctcttcccacctccg C/T tggactgtgagttccatgag | 5856 | | |
| ALDH1L1 | 95 | intron 20 + 1564 | cccaggaaccaggaacagtg G/A ggagccatgacccggccctg | 5857 | | |
| ALDH1L1 | 96 | intron 20 + 1873 | taagtgttaaaacatcattt G/A tgtatgtatgaaaaatattg | 5858 | | |
| ALDH1L1 | 97 | intron 20 + 2427 | aataggattggatggacttg G/C gatcaggtctcagctctgtc | 5859 | | |
| ALDH1L1 | 98 | intron 20 + 2458 | cagctctgtcacctgccaac C/T ggcggccccatttccatcaa | 5860 | | |
| ALDH1L1 | 99 | intron 20 + 2544 | ccaggtgggagagaaatatg C/T agcgtggtgacacccatcac | 5861 | | |
| ALDH1L1 | 100 | intron 20 + 2573 | gacacccatcacacgggtgc C/T gtgacccggtgcttatgtcg | 5862 | | |
| ALDH1L1 | 101 | intron 20 + 2574 | acacccatcacacgggtgcc G/A tgacccggtgcttatgtcgg | 5863 | | |
| ALDH1L1 | 102 | intron 21 + 33 | agccacctgttttcacagac G/A tggaagaccacatgttcata | 5864 | | |
| ALDH1L1 | 103 | exon 21 + 87 | ccttcgggcctgtcatgatc A/G tctctcggtttgctcatggg | 5865 | | |
| ALDH1L1 | 104 | intron 21 + 323 | ccatgcattaaaccaccccc C/G acactgagtcgcttggaata | 5866 | | |
| ALDH1L1 | 105 | intron 21 + 361 | ataatcagagatttatttta C/G tcacggtctaggttcaatga | 5867 | | |
| ALDH1L1 | 106 | intron 21 + 478 | gtcttccgcgaggcttcctc C/A gcctggcagcctcccggttg | 5868 | | |
| ALDH1L1 | 107 | intron 23 + 1086 | caacccaatcttccccccgg C/T gctgcagccccgcacatttt | 5869 | | |
| ALDH1L1 | 108 | intron 22 + 235 | gggcctgcacgacacactcc A/C gccaggaggcactgggggcc | 5870 | | |
| ALDH1L1 | 109 | intron 22 + 313 | atagaagggagcacttgccc G/A tgaagacccaggggcccgtg | 5871 | | |
| ALDH1L1 | 110 | intron 22 + 1214 | tgggcccacttatgaatcct G/C cccgagttccctcagctccc | 5872 | | |
| ALDH1L1 | 111 | intron 22 + 1226 | tgaatcctcccggacttccc T/C cagctccctcctaaccctag | 5873 | | |
| ALDH1L1 | 112 | intron 22 + 1623 | ggggcttcccactgtccaga C/G aaggcggtgggagctgggga | 5874 | | |
| ALDH1L1 | 113 | intron 22 + 1688 | attctggggagtcctggccc A/G ctatccactgccagggataa | 5875 | | |
| ALDH1L1 | 114 | 3′flanking + 145 | gagagacaggacgaaatggg C/T gtgggtcatctcggccccca | 5876 | | |
| ALDH1L1 | 115 | 3′flanking + 239 | tgggaaacaggtgggaagac G/A gggattgagctgggtgagcc | 5877 | | |
| ALDH1L1 | 116 | 3′flanking + 288 | ggaagcagctcagactccct C/T agcagatggccccggcccct | 5878 | | |
| ALDH1L1 | 117 | 3′flanking + 1513 | agggtcgcctcagaccccgg A/C gtgctcctgccatgtccagc | 5879 | | |
| ALDH1L1 | 118 | 3′flanking + 1707 | cggtgccccttgccctagca C/T gtgccacttatatcagaaca | 5880 | | |
| ALDH1L1 | 119 | 3′flanking + 1709 | gtgggacttgccctagcacg C/T gccacttataccagaacaga | 5881 | | |
| ALDH1L1 | 120 | 3′flanking + 1745 | acagatgagtccatgtcaac C/T gcttcctgagttccctttgt | 5882 | | |
| ALDH1L1 | 121 | 3′flanking + 1843 | ctgcctctcagcccacaccc G/A ggccgctcacactcctccca | 5883 | | |
| ALDH2 | 1 | intron 3 + 1766 | aaattggtggctcatcctgc C/Δ tggcccccggtccccgcggc | 5884 | | |
| ALDH2 | 2 | intron 8 + 52 | gaaggtagccctccccacct G/C tgttgtcgctccagccgatc | 5885 | | |
| ALDH2 | 3 | intron 8 + 69 | cctgtgttgtccctccaccc G/A accctgtcgcccccccggtg | 5886 | | |
| ALDH2 | 4 | intron 9 + 5197 | gctttcttatgaccttggtc C/A attccccagctgtcgggccg | 5887 | | |
| ALDH2 | 5 | intron 11 + 114 | gagctgggctcagtttctcc T/C gggtcagggtgtgaggtcga | 5888 | | |
| ALDH2 | 6 | 3′flanking + 411 | ggatatgatctctgcccctc T/C tctgctgtgggcaaacagct | 5889 | | |
| ALDH2 | 7 | 3′flanking + (432-433) | tctgctgtgggtaaacagct TC/Δ tgtttcatgcatttactttt | 5890 | | |
| ALDH2 | 8 | 3′flanking + 488 | ccaataagaatgtgcttgaa G/T gcttcaggcacccaaccggt | 5891 | | |
| ALDH3A1 | 1 | 5′flanking − 758 | ctgcaggcgggtgagggtgg C/A gggaagcgcctggtgagagg | 5892 | | |
| ALDH3A1 | 2 | 5′flanking − 308 | agtccccaaagctggaagag C/T cccatgccaggctgaatcaa | 5893 | | |
| ALDH3A1 | 3 | 5′flanking − 294 | gaagagctccatgccaggct G/A aatcaatcagcagcccccac | 5894 | | |
| ALDH3A1 | 4 | 5′flanking − 3 | gtcccctcttggctcttgcc G/A ctccaggagncccagttacc | 5895 | | |
| ALDH3A1 | 8 | intron 1 + 2323 | actgtctcctttctttcgga C/T ctttgcgatgtttacaatac | 5896 | | |
| ALDH3A1 | 6 | intron 1 + 2499 | cccgatttgccactatactt T/C cgtgtattcctagcaggaat | 5897 | | |
| ALDH3A1 | 7 | intron 1 + 2943 | caggggctaccaaggcaccc A/G gggcccacgcgtctgagtga | 5898 | | |
| ALDH3A1 | 8 | intron 5 + 72 | cacacatgactgcacctaat G/C ccgtgggtccactctgagta | 5899 | | |
| ALDH3A1 | 9 | intron 7 + 633 | cgcgtgggggtctctgcgcc G/A tccaactctggcttgtttcc | 5900 | | |
| ALDH3A1 | 10 | exon 8 + 36 | cggacgtggacccccagtcc C/G cggtgatgcaagaggagatc | 5901 | | |
| ALDH3A1 | 11 | intron 9 + (40-41) | gcggcccccctctgggcccc (C) agggcggggcacactcaccc | 5902 | | |
| ALDH3A1 | 11 | intron 9 + (40-41) | gcggcctccctctgggcccc agggctgggcacactcaccc | 5903 | | |
| ALDH3A1 | 12 | intron 9 + 322 | cacagtgtggatgccctggg G/Δ acaccctagacattggccac | 5904 | | |
| ALDH3A2 | 1 | intron 1 + 39 | gggtgtggggaaacgggccc C/T cgccgcgcactcgtccactg | 5905 | | |
| ALDH3A2 | 2 | intron 3 + 2491 | tgccgcgaagaaaccggcac T/A gctgagttccacaggcagct | 5906 | | |
| ALDH3A2 | 3 | intron 3 + 2595 | ttctgtacatcaacttgtga T/A ggattgaggccagctctcct | 5907 | | |
| ALDH3A2 | 4 | intron 3 + 2775 | taccgctttgcccctgacca G/A gggcaaactcttcaataact | 5908 | | |
| ALDH3A2 | 5 | intron 3 + 3424 | aggcacttctgcacacaccc G/A cgcctcaggcatttcccctg | 5909 | | |
| ALDH3A2 | 6 | intron 3 + 3676 | atgttgaagagattcctgat G/A ttagacgttaggattcattt | 5910 | | |
| ALDH3A2 | 7 | intron 4 + 481 | tagaaaataagaggtttcac G/T ttctctctgctaaatccggt | 5911 | | |
| ALDH3A2 | 8 | intron 4 + 769 | acccggctttatacctgaac G/A tcttgcaggcagagccaaaa | 5912 | | |
| ALDH3A2 | 9 | intron 4 + 796 | aggcagagccaaaagccaca A/G ccaggagagtctgtaccgaa | 5913 | | |
| ALDH3A2 | 10 | intron 5 + 254 | attagttgtggcatatactt T/G tttcaaaaaagttaaataat | 5914 | | |
| ALDH3A2 | 11 | intron 6 + 137 | aatcctgctttctggtatac T/C gtacctgtagcttttgttat | 5915 | | |
| ALDH3A2 | 12 | intron 6 + 923 | aggcgaatgaatggcaagag G/A aaggggctatcctgattagc | 5916 | | |
| ALDH3A2 | 13 | intron 7 + 331 | tgctcttttctgatgaatcc A/Δ cacggcattgctgaataaca | 5917 | | |
| ALDH3A2 | 14 | intron 8 + 643 | tttagaacatgacctgcctg C/T ctctcccacatgtcagatga | 5918 | | |
| ALDH3A2 | 15 | intron 8 + 666 | ctcccacaggtgagatgact G/A actcagctctttatttctcc | 5919 | | |
| ALDH3A2 | 16 | intron 9 + 2129 | tgttttcatttttaaaaaaa G/T gtttgactttggaattcatg | 5920 | | |
| ALDH3A2 | 17 | exon 10 + (1894-1895) | ttggcttgtctactaataca CA/Δ tctgcttcaaaatgaacaca | 5921 | | |
| ALDH3A2 | 18 | 3′flanking + 31 | gtaggtgtcaactttttttt T/Δ ctcattttaaaattcttagc | 5922 | | |
| ALDH3A2 | 19 | 3′flanking + 106 | gtgtgttggccgtggtggtt G/A gcagctatagtaaataggtt | 5923 | | |
| ALDH3A2 | 28 | 3′flanking + 1630 | aaaagcacgtgcgaaacaca A/G ttaatcatgtcttaccgtat | 5924 | | |
| ALDH3B1 | 1 | 5′flanking − 1485 | cggcctgtccacacccacac C/T agcttgaacctcatccccac | 5925 | | |
| ALDH3B1 | 2 | intron 1 + 464 | catgaatgactctgggaaag A/G atcattcttagcaatggact | 5926 | | |
| ALDH3B1 | 3 | intron 1 + 2269 | aaatggaatccaaacagcaa G/C agacctcccctcaccggtca | 5927 | | |
| ALDH3B1 | 4 | intron 2 + 1349 | actgagcttctgccaccggc C/T gcctgccggccttcatgaga | 5928 | | |
| ALDH3B1 | 5 | intron 2 + 1820 | tccgtgtggaaggcaccttc C/G cccagcctcagtggctagga | 5929 | | |
| ALDH3B1 | 6 | intron 2 + 2046 | aacctcaggcgctgcctcag C/G cagggagccagcctggcccc | 5930 | | |
| ALDH3B1 | 7 | intron 2 + 2939 | aagcacgcactgaacatgga G/A tgagtgagtgaecgaatgaa | 5931 | | |
| ALDH3B1 | 8 | intron 3 + 7 | tgcccaagaacctggtgagc C/T ggccgggctgaggcgggcag | 5932 | | |
| ALDH3B1 | 9 | intron 4 + 36 | gccccttccggccaccctcc T/C ccgctcgaggcctcaggccc | 5933 | | |
| ALDH3B1 | 10 | intron 6 + (116-117) | attctcctctctctctctct CT/Δ ggaccaggctgggagcagtc | 5934 | | |
| ALDH3B1 | 11 | intron 6 + 263 | cagaccctcatacgtgaccc T/C gctgccccccaggctcttag | 5935 | | |
| ALDH3B1 | 12 | intron 6 + 1298 | gtagacagagctggactcca T/G ccttgggtgataagggatcc | 5936 | | |
| ALDH3B1 | 13 | intron 6 + 1411 | gccagggtcacaagcagagg C/T gggaggsgccaaggggtttg | 5937 | | |
| ALDH3B1 | 14 | exon 7 + 185 | acctgcgtggcccccgacta C/T gtcctatgcagccctgagat | 5938 | | |
| ALDH3B1 | 15 | exon 7 + 339 | tgcgggcattgctgggctgc G/A gcgtgtggccattgggggcc | 5939 | | |
| ALDH3B1 | 16 | intron 7 + 249 | ccagggctccagggctcagc G/A tgctaagatgaactcccatc | 5940 | | |
| ALDH3B1 | 17 | intron 7 + 277 | atgaactcccatcccaccac C/T ggctatcctgaaaggctgta | 5941 | | |
| ALDH3B1 | 18 | intron 7 + 498 | gaccaaggtcgggggattct C/T tgtgtcccacaggccctgag | 5942 | | |
| ALDH3B1 | 19 | intron 8 + 14 | cagccaggtgggggtgcggc C/T gggctgggcagggtcaggag | 5943 | | |
| ALDH3B1 | 20 | intron 8 + 49 | caggagcccgcagtgggcag C/T acaagtggtggcagcagggg | 5944 | | |
| ALDH3B1 | 21 | intron 8 + 111 | tcaggactttgggatggtgg A/T cctcttagctctgtctctgc | 5945 | | |
| ALDH3B1 | 22 | intron 8 + 3219 | atcctgatggggctcaaggc A/G gcctcacgcacatcctgttc | 5946 | | |
| ALDH3B1 | 23 | exon 9 + 33 | gtgctgacccagaccagcag C/T gggggcttctgtgggaagga | 5947 | | |
| ALDH3B1 | 24 | intron 9 + 946 | tcccaggcccccgagctgac C/A cttcttggtggccgtggccc | 5948 | | |
| ALDH3B1 | 25 | intron 9 + 1067 | aggctccccaagcctgggtc C/T ctcttgcccccacccactct | 5949 | | |
| ALDH3B1 | 26 | exon 10 + 137 | ccgcaatcgccgcgccgcct G/A aggatgctgctggtggccat | 5950 | | |
| ALDH3B1 | 27 | exon 10 + 397 | cgctcccaaccatcagagcc G/A aggtgggaggcatgggaaac | 5951 | | |
| ALDH3B1 | 25 | exon 10 + 1198 | ctcttccccatgctgctcat C/T ctcctgggccccatccactc | 5952 | | |
| ALDH3B1 | 29 | exon 10 + 1475 | caggggtggaggtgagtttc G/A tctcctgtctctctggctga | 5953 | | |
| ALDH3B1 | 30 | 3′flanking + 15 | cctggcaatacttacatcct A/G gtgatttgctttctgtgcat | 5954 | | |
| ALDH3B1 | 31 | 3′flanking + 60 | caacagggctgtggaccaag G/C ccctggcgttgggtaacaat | 5955 | | |
| ALDH3B2 | 1 | intron 1 + 98 | agggaaggggatgtgtgccc G/A tggcccgtgggtcagggggc | 5956 | | |
| ALDH3B2 | 2 | intron 1 + 157 | atggctccaggggccatggg T/C acggggcttcctcaggacag | 5957 | | |
| ALDH3B2 | 3 | intron 1 + 354 | tctgtggacagacaaggatt C/G ggtcgggggcaccagggctg | 5958 | | |
| ALDH3B2 | 4 | intron 1 + 851 | tatgacaggtccatcaggcc T/G caccttcctgtgtgtcttat | 5959 | | |
| ALDH3B2 | 5 | intron 1 + 894 | ctcagcatctgcccccacag T/G gcttttgcacacgttggttc | 5960 | | |
| ALDH3B2 | 6 | intron 1 − 463 | aaagaaccctcccagtccct C/G gtttagtcccagaagggagg | 5961 | | |
| ALDH3B2 | 7 | exon 2 + 61 | gccttcaactgagggcgcac G/A cggccggccgagttcggggc | 5962 | | |
| ALDH3B2 | 8 | intron 2 + 9 | ggacctgcataaggtgggcc A/G tggagaatgggccggggcag | 5963 | | |
| ALDH3B2 | 9 | intron 2 + 23 | gggccgtggagagtgggccg G/C ggcaggggctggagcagcgt | 5964 | | |
| ALDH3B2 | 10 | intron 2 + (180-181) | ttcactcctgaacactcaca (A) gccaccctgtgatgcagact | 5965 | | |
| ALDH3B2 | 10 | intron 2 + (180-181) | ttcactcctgaacactcaca gccaccctgtgatgcaggct | 5966 | | |
| ALDH3B2 | 11 | exon 3 + 72 | gactacgctctcaagaacct T/G caggcctggataaagcatga | 5967 | | |
| ALDH3B2 | 12 | intron 8 + 375 | ctgcagcatcctaacctcac C/T gtcgcgactcaaggctgccg | 5968 | | |
| ALDH3B2 | 13 | intron 8 + 463 | aatcacccccatggcacccc G/A accgtcactgacagggtgct | 5969 | | |
| ALDH3B2 | 14 | exon 9 + 33 | atgctggagcggaccagcag C/A ggcagctttgcaggcaatga | 5970 | | |
| ALDH3B2 | 15 | exon 10 + 428 | aggtgtcctcactcacccca C/T cctcccnaattccagccctt | 5971 | | |
| ALDH5A1 | 1 | 5′flanking − 1303 | gaaattgattaaactctact G/A ttatcgcttctgccatatgt | 5972 | | |
| ALDH5A1 | 2 | 5′flanking − 301 | gtgaaaaggtgacagcagtc C/T gcaggtgcatctactggcga | 5973 | | |
| ALDH5A1 | 3 | 5′flanking − 221 | ggtcgcgccaggagagaagc C/T gcgcggcgcttagggcaagg | 5974 | | |
| ALDH5A1 | 4 | 5′flanking − 175 | agggcggcgcggcggtgcag C/G gagaaagacgcggagagagc | 5975 | | |
| ALDH5A1 | 5 | 5′flanking − 174 | gggcggcgcggcggtgcagc G/A agaaagacgcagacaaacgg | 5976 | | |
| ALDH5A1 | 6 | exon 1 + 106 | gcggcctggtccctgcctcc G/C ggcctgcgcccgcggcggcc | 5977 | | |
| ALDH5A1 | 7 | intron 1 + 326 | cctaaccgtggaggggcggg G/A agaaaggggaggggtgtcac | 5978 | | |
| ALDH5A1 | 8 | intron 1 + 5551 | gtctgtacaaaaaaaatttt T/G ttttaattagctcagcatga | 5979 | | |
| ALDH5A1 | 9 | intron 1 + 5585 | gtacaaaaaaaatttttttt T/Δ aattagctgagcatgatgat | 5980 | | |
| ALDH5A1 | 10 | intron 2 + 306 | gttttggttgtttttttttt T/Δ aaacttgtttttgtacattt | 5981 | | |
| ALDH5A1 | 11 | exon 3 + 107 | cggagacattatccacaccc C/T ggcaaaggacaggcgggccc | 5982 | | |
| ALDH5A1 | 12 | intron 3 + 201 | gtggtggcagtgagtggaat G/T atgcatttctaatgcctgca | 5983 | | |
| ALDH5A1 | 13 | exon 4 + 42 | atcacccggaaggtgggggc C/T gccctggcagccggctgtac | 5984 | | |
| ALDH5A1 | 14 | intron 4 + 2306 | atcgtcjcttataaatcagt T/C tgctaggtataaaatccttg | 5985 | | |
| ALDH5A1 | 15 | intron 4 + (2334-2346) | tataaaatccttggctcaca (T)11-13 acttgattatcttaaatgta | 5986 | | |
| ALDH5A1 | 16 | intron 4 + 2456 | tataagtcaacttttttttt T/Δ acctagatacacaaaagtgt | 5987 | | |
| ALDH5A1 | 17 | intron 4 + 2501 | tttggtttttttcggccttt A/G tctttaaaaaccaataatgt | 5988 | | |
| ALDH5A1 | 18 | intron 4 − (64-46) | atctatttatttctcttttc (T)16-18 cagtttggtaaattgttggc | 5989 | | |
| ALDH5A1 | 19 | intron 4 − 27 | ttcagtttggtaaattgttg G/C cacatgtttcctgtttctct | 5990 | | |
| ALDH5A1 | 20 | intron 5 + (4621-4624) | tttgaatagataaacactta CTTA/Δ tatggttgaaaaattaacac | 5991 | | |
| ALDH5A1 | 21 | intron 5 + (4677-4678) | accatgacaagtctcaccct (C) accccaaccctgactcacct | 5992 | | |
| ALDH5A1 | 21 | intron 5 + (4677-4678) | accatgacaagtctcaccct accccaaccctgactcactc | 5993 | | |
| ALDH5A1 | 22 | intron 7 + (432-443) | aaaaaatgatgttaaaaggc (A)10-12 tgaaaacaaaaaagtcattt | 5994 | | |
| ALDH5A1 | 23 | intron 7 + (3243-3244) | cagtccttgtgtgtgtgtgt GT/Δ cccccaaacacactgctgga | 5995 | | |
| ALDH5A1 | 24 | intron 7 + 4957 | tttttgaaaaaaaaaaaaaa A/Δ tggaactagttatacttttc | 5996 | | |
| ALDH5A1 | 25 | intron 8 + 2717 | gatcacctggaactcacagg C/T gtggtaggagacgtgcagcc | 5997 | | |
| ALDH5A1 | 26 | 3′flanking + 2711 | cagtgagtgccttggggaag G/A agccagcatgtgaaatcatg | 5998 | | |
| ALDH5A1 | 27 | 3′flanking + 2777 | gtccatggtgtgcgcttata G/A aatgtttgctaaactgaact | 5999 | | |
| ALDH6A1 | 1 | 5′flanking − 1303 | ctctaaagcagaaccaagag G/C aaaagcatgggagtatacca | 6000 | | |
| ALDH6A1 | 2 | 5′flanking − (1273-1270) | ggagtctaccaaaacaactt AATT/Δ gttacttgaaatgacttgca | 6001 | | |
| ALDH6A1 | 3 | intron 1 + 437 | tgcccttgctcccttccccc A/T ccctacttcactatccatgg | 6002 | | |
| ALDH6A1 | 4 | intron 1 + 835 | gttcccaccccaaaatgagc T/Δ cttctagtgctacacaccgt | 6003 | | |
| ALDH6A1 | 5 | intron 1 + 1294 | atattccttgctgcgatcct T/C gttctgttctagtatctttt | 6004 | | |
| ALDH6A1 | 6 | intron 1 + 1447 | gagtcattgagaaccttaag A/G aagtattttgtccttttcca | 6005 | | |
| ALDH6A1 | 7 | intron 1 + 2536 | agtcttgccatctctttcta T/C gttagccactgacataggct | 6006 | | |
| ALDH6A1 | 8 | intron 1 + 2703 | caggagagcaaggacttcct G/T ataaagcatatagcaagtag | 6007 | | |
| ALDH6A1 | 9 | intron 2 + 2802 | gcaacaatgctaatgggtgt T/C tcttagcaaatgaagaaaag | 6008 | | |
| ALDH6A1 | 10 | intron 2 + 2333 | gtttgtttgtttgtttgttt G/Δ tttttttcagccaactgtaa | 6009 | | |
| ALDH6A1 | 11 | intron 4 + 138 | gactctctcccttgtactgc A/G tctcctccagtcttattctt | 6010 | | |
| ALDH6A1 | 12 | intron 4 + 200 | aaagaggaacattcttgcat T/C aatttctatttgtgtgtctt | 6011 | | |
| ALDH6A1 | 13 | intron 5 + 291 | ggcaagtcagtgtaccctgc G/A ccccttcattggcctgaacc | 6012 | | |
| ALDH6A1 | 14 | intron 7 + 209 | tcccgggttcaagcgattct G/A ctgcctcagcctccccacta | 6013 | | |
| ALDH6A1 | 15 | intron 8 + 287 | gcctcctgagcagcttggac C/T acaggtgcgggccaccacct | 6014 | | |
| ALDH6A1 | 16 | intron 9 + 877 | gatatcaaaatataaacata C/T agacatatttgggaggcaaa | 6015 | | |
| ALDH6A1 | 17 | intron 9 + 885 | aatataaacatacagacata T/G ttgggaggcaaaggagtgaa | 6016 | | |
| ALDH6A1 | 18 | intron 11 + 40 | ttttgtcttttcctttaaga A/C attttcttaaagatattcag | 6017 | | |
| ALDH6A1 | 19 | 3′flanking + 520 | cctgcaaagttttctttagc C/T cctcttttatcccacaatac | 6018 | | |
| ALDH6A1 | 20 | 3′flanking + 1026 | cgtgttggtcaggctggtct T/C gaactcctgacctcaggtga | 6019 | | |
| ALDH6A1 | 21 | 3′flanking + 1035 | caggctggtctcgaactcct G/C acctcaggtgatccgcctgc | 6020 | | |
| ALDH8A1 | 1 | 5′flanking − (837-836) | gctgaacattgttaatatat (AT) tcattagccaattgtgttcc | 6021 | | |
| ALDH8A1 | 1 | 5′flanking − (837-836) | gctgaacattgttaatatat tcattagccaattgtgttcc | 6022 | | |
| ALDH8A1 | 2 | 5′flanking − 702 | gggatctgaagcccttgcta C/T atgtgtcacacatgtttttg | 6023 | | |
| ALDH8A1 | 3 | 5′flanking − 642 | gcacatctaggaagatgtga G/A cagccactgtggccccggtt | 6024 | | |
| ALDH8A1 | 4 | 5′flanking − 84 | atgctctctgagagcgtcag G/T tgccctcccacattcactga | 6025 | | |
| ALDH8A1 | 5 | intron 1 + 5437 | gcattggttgaaatggagcg T/C gtttctttgtttctatggta | 6026 | | |
| ALDH8A1 | 6 | intron 1 + (5836-5855) | gtcagaatccatctaaaaaa (CAAAA)4-5 atgagctgtgtggagacct | 6027 | | |
| ALDH8A1 | 7 | ercn 3 + 146 | cactacacggtgcgggcccc G/T gtgggagtcggtgagtgctg | 6028 | | |
| ALDH8A1 | 8 | intron 4 + 1033 | aggtctttttgctatgtcac C/T ccacggccagggcaggagtg | 6029 | | |
| ALDH8A1 | 9 | intron 4 + 1037 | ctttttgctatgtcacccca C/T ggccagggcaggagtgctgg | 6030 | | |
| ALDH8A1 | 10 | intron 4 + 1662 | tctctcctgagaccaagaac G/A tctggatagatgatgagtta | 6031 | | |
| ALDH8A1 | 11 | intron 4 + 2046 | agtcctgggcatttaaacag A/C cttgacagataaacttcctt | 6032 | | |
| ALDH8A1 | 12 | intron 6 + 1146 | ttttccagatgcaagagact C/G ccttgttctctctccttctg | 6033 | | |
| ALDH8A1 | 13 | intron 6 + 1744 | ttcttcttcttcttcttctt C/T tttcttttttaacatgtact | 6034 | | |
| ALDH8A1 | 14 | intron 6 + 9802 | tgagtgtgaattctaacttt A/T ctgtttattagctctatgaa | 6035 | | |
| ALDH8A1 | 15 | exon 7 + (1089-1098) | tacagtgagaccttgtcttt (A)9-10 tgctgcaaaaccaaaaataa | 6036 | | |
| ALDH8A1 | 16 | 3′flanking + 848 | ctcagctgagtccccttgac T/C ttaatcactttagtgaagaa | 6037 | | |
| ALDH9A1 | 1 | exon 1 + 121 | actgtgtggggtatggcggg G/A tggtggggagaatgtggtgt | 6038 | | |
| ALDH9A1 | 2 | intron 1 + 67 | cgcggatttcccggccagcc C/G ccgtttcctgtgttctgcag | 6039 | | |
| ALDH9A1 | 3 | intron 1 + 103 | tgcagcgttgacttgagcac A/G agacagtgacagtggagagt | 6040 | | |
| ALDH9A1 | 4 | intron 1 + 1818 | gaatttttgagaaaaaaaaa A/Δ tgttcctttggggttgcctt | 6041 | | |
| ALDH9A1 | 5 | intron 2 + 5891 | tcaggaacaggaagtaaaga G/A gtttacatttctaaatttct | 6042 | | |
| ALDH9A1 | 6 | intron 2 + 6398 | atcaaaaacanttgtctgat T/G atcgtgctctgaacctgcct | 6043 | | |
| ALDH9A1 | 7 | intron 2 + 9677 | atgacgctgagtttggtgct A/G ttcttttgtttttcttgcct | 6044 | | |
| ALDH9A1 | 8 | intron 2 + 9991 | gggagaagtgagggacctac C/T cttggcttctaatctttcat | 6045 | | |
| ALDH9A1 | 9 | intron 2 + 10198 | ttgtcagagacatctttgat A/G atccttacgtactatatcag | 6046 | | |
| ALDH9A1 | 10 | intron 2 + 10256 | ttagtagataactttttttt T/Δ gtaaggatggagaataatag | 6047 | | |
| ALDH9A1 | 11 | intron 2 + 11382 | catattcaattcttttatgt T/C ctttagaccaaagaaaggca | 6048 | | |
| ALDH9A1 | 12 | intron 2 + 11455 | taaacctttaagctcatcat C/T ggaccatctattgaatttct | 6049 | | |
| ALDH9A1 | 13 | intron 2 + 12044 | atttaaagtgaaagctattt C/T tagttttaaaaattgagcag | 6050 | | |
| ALDH9A1 | 14 | intron 3 + 334 | ctatttagcaaActtttttt T/Δ gacagtgtataaagttttca | 6051 | | |
| ALDH9A1 | 15 | intron 3 + 368 | gttttcaacaattgatattg G/Δ aaggttggtagggcctagga | 6052 | | |
| ALDH9A1 | 16 | intron 4 + 191 | ccctcaaggagcttatagtt T/A aggttgtacacaatcatgtc | 6053 | | |
| ALDH9A1 | 17 | intron 4 + 557 | tagaacaaattgtaatgtta A/G aaagcattactgttaggaca | 6054 | | |
| ALDH9A1 | 18 | intron 5 + 830 | agttcaagatgattttgtag G/C ttcagggcctagttgactta | 6055 | | |
| ALDH9A1 | 19 | intron 5 + 838 | atgattttgtaggttcaggg C/T ctagttgacttagcatgcaa | 6056 | | |
| ALDH9A1 | 20 | intron 6 + 120 | agaaaagttgcacaaatagt A/C caaagaattcccatgtacct | 6057 | | |
| ALDH9A1 | 21 | intron 6 + 2569 | attaaaatctgctttaaata T/C ttttttgggggagaggacac | 6058 | | |
| ALDH9A1 | 22 | intron 8 + 1414 | ccgatcttcaaaaaattagc T/C gggggtggtggtgcacactg | 6059 | | |
| ALDH9A1 | 23 | intron 9 + 664 | aaagttcacatttttttttt T/Δ ataacttcatggtcaagagc | 6060 | | |
| ALDH9A1 | 24 | intron 9 + 2170 | taatgcacacattttttttt T/Δ cttcatagggacatccaacg | 6061 | | |
| ALDH9A1 | 35 | exon 11 + 587 | aaaacaaaaaacaaaaaaaa A/Δ ccttgttcctttataggttc | 6062 | | |
| ADH1 | 1 | (5′flanking region − 55) | atcatgtgtggaactggaat C/T gggtgttattcaagcaaaaaa | 6063 | | |
| ADH1 | 2 | (intron 1 268) | acatttgcggtaaagcgata A/G tttattccaagctaatcatg | 6064 | | |
| ADH1 | 3 | (intron 3 443) | aatgga g/c gctacatggctat G/A gctgaatgagcatgaccttt | 6065 | | |
| ADH1 | 4 | (intron 6 56) | tacaacttggaggatgcatt T/G aggctgcagaatatatgttt | 6066 | | |
| ADH1 | 5 | (intron 8 74) | gtctagcagaaaatgaaaag G/A tggaaggatgagaaaaatta | 6067 | | |
| ADH2 | 1 | (intron 2 340) | ctattttttaaagcgtgcat T/C cttacataagacttaaatat | 6068 | | |
| ADH2 | 2 | (intron 3 91) | aaggcaatgagagaggaaag T/G gcttgcacaaggtcaccgcg | 6069 | | |
| ADH2 | 3 | (intron 3 205) | atgtattgtacccttcaacc A/G ttatgtaccgagtatctact | 6070 | | |
| ADH2 | 4 | (intron 7 108) | acaattgacaaggcaagatt T/C tgaaaacaaatcaaaaataa | 6071 | | |
| ADH2 | 5 | (intron 3 1721-1723) | actgcatagaaatttaagaa GAA/Δ cttgttttattcctctccag | 6072 | | |
| ADH2 | 6 | (3′untranslated region | gttaatgctttcccaccttc AG/Δ gggaaggatttgaattttga | 6073 | | |
| | | 2305-2306) | | | | |
| ADH3 | 1 | (5′flanking region − 254) | tgagagaagagaagcaggaa C/G ttgagagaggaggaagagag | 6074 | | |
| ADH3 | 2 | (intron 2 355) | tatgccttcttctatattat A/G caagacaaaaattttaggat | 6075 | | |
| ADH3 | 3 | (intron 3 32) | acactcagggaacatgcctt G/A gttcaccatcacaagattag | 6076 | | |
| ADH3 | 4 | (intron 4 6) | ctgcttgaaaaatgagtaag C/T ttctgatgctttctttgcac | 6077 | | |
| ADH3 | 5 | (coding region 453 | agcaccttctcccagtacac A/G gtggtggatgagaatgcagt | 6078 | | |
| | | (Thr 151 Thr)) | | | | |
| ADH3 | 6 | (coding region 815 | ttcgtttgaagtcatcggtc A/G gcttgacaccatggtatgat | 6079 | | |
| | | (Arg 272 Gln)) | | | | |
| ADH4 | 1 | (5′flanking region − 482) | acagccagagacccagaacc A/C tcagggctggttgatggact | 6080 | | |
| ADH4 | 2 | (5′flanking region − 437) | catcaggtgggacaaaaaga G/A tagctccttagcagtgacta | 6081 | | |
| ADH4 | 3 | (5′flanking region − 234) | actcaagcatatgtgcaacc A/G agtacatgaaaagaatttgt | 6082 | | |
| ADH4 | 4 | (5′untranslated region − 3) | ggtaagttaaatgggcgatt C/G tgaggagtagaaatttcctt | 6083 | | |
| ADH4 | 5 | (5′untraslated region − 2) | ttcaataaaagaaaaaagaa T/A ttaaaaaatcttggagctca | 6084 | | |
| ADH4 | 6 | (intron 1 707) | ttatatttgaaattaaaaat A/G taatttgaggctagaaaaaa | 6085 | | |
| ADH4 | 7 | (intron 5 619) | tcaaagagggatctcacaat T/C ggacatctcaacctgcttat | 6086 | | |
| ADH4 | 8 | (intron 5 1755) | tttacgcacacaattactca T/C taataaaaaatttaaaaaat | 6087 | | |
| ADH4 | 9 | (intron 5 3425) | actgagactctggagcaata T/C aitaagaatcatactgaaca | 6088 | | |
| ADH4 | 10 | (intron 1 1181-1189) | ggtaaactttaatacacctg (T)9-11 caagaaataaaaaatgtaat | 6089 | | |
| ADH4 | 11 | (intron 5 2828) | tccagtcaaagtcgacctaa A/Δ tttccaggagttgttcttcc | 6090 | | |
| ADH4 | 12 | (intron 7 15) | ttggtggtcagttttttttt T/Δ cttcatagctttaaattctt | 6091 | | |
| ADH5 | 1 | (5′flanking region − 115) | taactgctgtaaagttacac G/A g/a ggaagccctttcccgacaa | 6092 | | |
| ADH5 | 2 | (5′flanking region − 114) | aactgctgtaaagttacac g/a G/A ggaagccctttcccgacaaa | 6093 | | |
| ADH6 | 1 | (intron 3 249) | tgaaactggccttgaaagta C/A aaatgagacaaaaatttatg | 6094 | | |
| ADH6 | 2 | (intron 6 1072) | taacccctatactgtattgc A/G tcactttctaacaggcagct | 6095 | | |
| ADH6 | 3 | (coding region 885 | gtctgtgtggttgttggggt G/A ttgcctgccagtgttcaact | 6096 | | |
| | | (Val 295 Val)) | | | | |
| ADH6 | 4 | (intron 7 1292) | gttgagaaacactgcctagt C/A ccgtctgtggtcctggaatt | 6097 | | |
| ADH6 | 5 | (intron 7 1616) | ctatcacagaataatccgca T/C agaacactaagcagattacg | 6098 | | |
| ADH7 | 1 | (5′flanking region − 520) | tgtgcagacacagaaagttt T/C acttaactttctacacctaa | 6099 | | |
| ADH7 | 2 | (intron 1 361) | tcagtagcatgtgctgcact C/T gctgcagtagttcaatggga | 6100 | | |
| ADH7 | 3 | (intron 3 183) | aacctcaacctttagaaggc A/G aaccttacggtgtttataaa | 6101 | | |
| ADH7 | 4 | (intron 4 76) | tgaattgaattaattaatac G/A tgtatttgatgtatcaaaca | 6102 | | |
| ADH7 | 5 | (intron 6 615) | tggcatagcgtaaagagact T/A ggaaaaatggaataaagcca | 6103 | | |
| ADH7 | 6 | (intron 8 532) | aagtctaaccatatcaccaa T/C ttagtatgccattgtactat | 6104 | | |
| ADH7 | 7 | (intron 8 651) | gctctgatttatttcaagta G/A gccacaaaatttccttattt | 6105 | | |
| ADH7 | 8 | (intron 8 727) | ttcagatccctgtaagccag G/A tattatttttaccattttta | 6106 | | |
| ADH7 | 9 | (intron 8 1207) | tctccacatttggtctagcc T/C acaggatcatcatattatga | 6107 | | |
| ADH7 | 10 | (intron 8 1691) | tccctcatctcattgcccac G/A ctcattgctttaattcagtc | 6108 | | |
| ADH7 | 11 | (3′untranslated region 13 | atttacattttgtaaggcta T/C aattgtatcttttaagaaaa | 6109 | | |
| ADH7 | 12 | (3′untranslated region 14 | gatatagtaaatgcatctcc T/C agagtaatattcacttaaca | 6110 | | |
| ADH7 | 13 | (3′untranslated region 15 | aaacacttgttatgagttaa C/G ttggattacattttgaaatc | 6111 | | |
| ADH7 | 14 | (3′untranslated region 18 | aatataaacatagagctaga A/G tcatattatcatacttatca | 6112 | | |
| ADH7 | 15 | (3′flanking region 865) | tacatcaaaagaaataaatc C/T aagaaggaataaacacattt | 6113 | | |
| HEP27 | 1 | (5′flanking region − 191) | tcagcactctgtgtctagct A/T aaggtttgtaaatgcaccaa | 6114 | | |
| HEP27 | 2 | (5′untranslated region − 1) | gaacccatcaattccgtaca C/A attttggtgactttgaagag | 6115 | | |
| HEP27 | 3 | (intron 1 1941) | aaatttaccctaaccagcct G/C actctctgccactttctgtt | 6116 | | |
| HEP27 | 4 | (coding region 211 | ttgtgtgccacgtggggaag G/A ctgaggaccgggagcagctg | 6117 | | |
| | | (Ala 97 Thr)) | | | | |
| HEP27 | 5 | (intron 4 1070) | tgtctcagttcacaggatca T/C gactctttttctcgaaactg | 6118 | | |
| HEP27 | 6 | (3′flanking region 362) | ggctttgtgtgtgctccatt A/G tctgaactgggcctgctggg | 6119 | | |
| UGT1A1 | 1 | (5′flanking region − 1337 | tctttcccttttgacttcaa A/C tcagtcatcagaatttcccc | 6120 | | |
| UGT1A1 | 2 | (coding region 211 | cctcgttgtacatcagagac G/A gagcattttacaccttgaag | 6121 | | |
| | | (Gly 71Arg)) | | | | |
| UGT1A1 | 3 | (intron 1 2925) | gcatttgggaagggaaaatc T/G aattaaaagcctaaactaaa | 6122 | | |
| UGT1A1 | 4 | (intron 1 3442) | agactcggccttttccagat G/T agcttcagtgtaagagtggg | 6123 | | |
| UGT1A1 | 5 | (intron 1 3512) | ttaagtaagccatttaccaa C/T gctcagaagaaagaacttga | 6124 | | |
| UGT1A1 | 6 | (intron 1 3665) | tcttgctacaaaccaaaaaa T/C gcagcatggtggtggggagg | 6125 | | |
| UGT1A1 | 7 | (intron 2 15) | cagacagtaagaagattcta T/C accatggcctcatatctatt | 6126 | | |
| UGT1A1 | 8 | (intron 4 574) | agatttaaaactccaattta C/T ataaaaagttcccataatag | 6127 | | |
| UGT1A1 | 9 | (3′flanking region 125) | tatagaggttcacacacaca C/T gccttcattgcgtgtgcatg | 6128 | | |
| UGT2A1 | 1 | (5′flanking region − 1602) | ataacatcttctgcagagaa A/C cttcaatggaaatacactca | 6129 | | |
| UGT2A1 | 2 | (5′flanking region − 1480) | tacagattatctttggtgat G/C ggagagcttagaagagacat | 6130 | | |
| UGT2A1 | 3 | (5′flanking region − 1406) | atttcagaagatttattaac A/T tgaaaaggatcactctg c/t tt | 6131 | | |
| UGT2A1 | 4 | (5′flanking region − 1388) | acatggaaaggatcactctg C/T ttattcacagacatatgcat | 6132 | | |
| UGT2A1 | 5 | (5′flanking region − 935) | aaattattcaatctctttgg G/A cagtggtttctttttctttg | 6133 | | |
| UGT2A1 | 6 | (intron 1 535) | cattgatcaggctgatttat C/T catgctaagcttatttaatt | 6134 | | |
| UGT2A1 | 7 | (intron 1 642) | tatattgatcatgttgatac A/C tttatacacatatttgtcta | 6135 | | |
| UGT2A1 | 8 | (intron 1 1448) | aggtgcttacaggcaacatc C/T acatagcagtctgtggctgg | 6136 | | |
| UGT2A1 | 9 | (intron 1 2000) | gacacattagcttcttttct A/G cagatctctgttctaaaaca | 6137 | | |
| UGT2A1 | 10 | (intron 1 3118) | cttaaaattctttaatgaaa T/G cattgcaacaaatttatatc | 6138 | | |
| UGT2A1 | 11 | (intron 1 3191) | ataaatagaacaactcccta A/T gtttacttctctgcagtgga | 6139 | | |
| UGT2A1 | 12 | (intron 1 3770) | atcaccagataatttactat C/T cattaaggagtaggtcatca | 6140 | | |
| UGT2A1 | 13 | (intron 1 4584) | tgattggttagaatctttga A/C aaatcttctagtatcattcc | 6141 | | |
| UGT2A1 | 14 | (intron 1 4854) | tactctgtgcattgttaata G/A cctatcacttgtggtctgcc | 6142 | | |
| UGT2A1 | 15 | (intron 1 − 19146) | ctgtttaaattctcattcaa C/T ggccacatggttaaaataaa | 6143 | | |
| UGT2A1 | 16 | (intron 1 − 19085) | tagacaaagaccctttcaat A/C aacaaagttagaaatgtgtt | 6144 | | |
| UGT2A1 | 17 | (intron 1 − 18346) | atggcaatatttttagaaat G/A ttaactcccaataatgaata | 6145 | | |
| UGT2A1 | 18 | (intron 1 − 18218) | tatatcattattttaactta T/G agatagcactagccctaatt | 6146 | | |
| UGT2A1 | 19 | (intron 1 − 17937) | ctcctaataatttggactca C/T catacttattcagcactatc | 6147 | | |
| UGT2A1 | 20 | (intron 1 − 12585) | ttccacacagggacaagtca A/G cagaggaaatttttcttgct | 6148 | | |
| UGT2A1 | 21 | (intron 1 − 11430) | eacaaaggtttattttctta C/G agttctgatggctagaggtc | 6149 | | |
| UGT2A1 | 22 | (intron 1 − 10761) | tttaaaatatgcatgtattt T/G ccacttttaaaaactatatc | 6150 | | |
| UGT2A1 | 23 | (intron 1 − 381) | aaatcctccctccttccttc C/T tttcccaggccccactctac | 6151 | | |
| UGT2A1 | 24 | (intron 1 − 329) | ttccctttctccttttctcc A/G tctctctctcttcctctctc | 6152 | | |
| UGT2A1 | 25 | (intron 1 − 41) | ttttctcctcagcaaacata T/A aagctaatttcctccatcca | 6153 | | |
| UGT2A1 | 26 | (intron 2 263) | caccttgatactggacttgg T/C gggacagaaaaccagatcat | 6154 | | |
| UGT2A1 | 27 | (intron 2 454) | agaaagcccattgaaataag G/C cagggtttttaggttttaat | 6155 | | |
| UGT2A1 | 28 | (intron 2 554) | aaaaacttttttgagttgac A/T atggtgagtttagtttctga | 6156 | | |
| UGT2A1 | 29 | (intron 2 1113) | ctgcaggcaagctctagtga A/T tgtttattataggaaataat | 6157 | | |
| UGT2A1 | 30 | (coding region 922 (Gly308) | gtgttgtggtattttctgtg G/A gatcaatggtcaaaaacctt | 6158 | | |
| UGT2A1 | 31 | (intron 3 − 217) | aagcttagaagtgataaata T/C caaaacaataatactatcct | 6159 | | |
| UGT2A1 | 32 | (intron 3 − 194) | aaacaataatactatagtgg G/A tagactattagtacaagact | 6160 | | |
| UGT2A1 | 33 | (coding region 1171 | acggagtccctatggtggga G/A ttcccatgtttgctgatcag | 6161 | | |
| | | (Val391Ile)) | | | | |
| UGT2A1 | 34 | (intron 5 1546) | tttttaaaattcagaaactc A/G g/a ttatggtgtattcttacaa | 6162 | | |
| UGT2A1 | 35 | (intron 5 1547) | ttttaaaattcagaaacct a/g G/A ttatggtgtattcttacaaa | 6163 | | |
| UGT2A1 | 36 | (intron 5 2505) | taattgacttttattaatac G/A tacatgttgtataagtcata | 6164 | | |
| UGT2A1 | 37 | (intron 5 2639) | tagactattacaaagttgtt A/G gttgctgacaattttgttca | 6165 | | |
| UGT2A1 | 38 | (intron 5 4009) | gaatccaggctggaactttt C/A ttccagacacaaaccaaaat | 6166 | | |
| UGT2A1 | 39 | (intron 5 4311) | atacagacactgtccttttc C/A tcacaaacatacagatgtgt | 6167 | | |
| UGT2A1 | 40 | (intron 5 4616) | acttttttatgtctacattt G/C atcatactgtgttcagcata | 6168 | | |
| UGT2A1 | 41 | (intron 5 4717) | tgcaagaattatattttctc C/A acgtaactatggccttaaac | 6169 | | |
| UGT2A1 | 42 | (coding region 1524 | gctatatttttggtcataca A/G tgttgtttgttttcctgtca | 6170 | | |
| | | (Gln508Gln)) | | | | |
| UGT2A1 | 43 | (3′untranslated region 1 | aaggagtttaacaaaaacac G/A tctcccatcctgtttccaaa | 6171 | | |
| UGT2A1 | 44 | (3′flanking region 685) | aatctagaaaataattatca T/C ttttataaaatttttagtca | 6172 | | |
| UGT2A1 | 45 | (intron 1 (−18967)-(−18965) | ctcccaattagattgattag TAT/Δ gagttcctggggttactggt | 6173 | | |
| UGT2A1 | 46 | (intron 1 (−18862)-(−18803) | aatacattcttcccccttca (AC)14-17 atacttactggcctatttat | 6174 | | |
| UGT2A1 | 47 | (intron 1 (−17463)-(−17447) | aaacttagaaacctctattc (A)16-27 gtaaagaaaatggcagagaa | 6175 | | |
| UGT2A1 | 48 | (intron 1 − 10860) | attcaatgcaactttttttt T/Δ gtaatggcagaattagaaca | 6176 | | |
| UGT2A1 | 49 | (intron 2 528-538) | ctgttaggaaacaattggtt (A)8-10 cttttttgagttgacA/Tatgg | 6177 | | |
| UGT2A1 | 50 | (intron 2 1514-1533) | ttgtgtgtatgtgtatgttt (GT)9-11 tattttaatgaattaatatc | 6178 | | |
| UGT2A1 | 51 | (intron 5 916-917) | gcttagtatattatatatat AA/Δ gtctatatatatagcttagt | 6179 | | |
| UGT2A1 | 52 | (intron 5 1163) | caatatttatgtcatttttt T/Δ ctcacatttactgtgtttcc | 6180 | | |
| UGT2A1 | 53 | (intron 5 3819-3038) | agacagacagacacacaaac (AC)8-12 tcaacacatgtaaactactc | 6181 | | |
| UGT2A1 | 54 | (intron 5 4785) | tatcttcaatgaaaataaaa A/Δ caaaaattgtccaatttctg | 6182 | | |
| UGT2B15 | 1 | (5′flanking region − 277) | ccgaacaggcaggagcctct C/A acttgccactgttcttaaca | 6183 | | |
| UGT2B15 | 2 | (intron 1 670) | catcaaagaaaataggggcc A/T aattaagggagagcacatat | 6184 | | |
| UGT2B15 | 3 | (intron 1 775) | ctaattatattaagatctta A/C gatgaaccaagacagtagta | 6185 | | |
| UGT2B15 | 4 | (intron 2 2183) | cagagtttcaccatgttggc C/T aggctggtcttgaactcctg | 6186 | | |
| UGT2B15 | 5 | (intron 2 2430) | tatttcaaaagaataagact C/G ttgccaaaaagtatcaagtg | 6187 | | |
| UGT2B15 | 6 | (intron 2 4806) | aaaaaattactccaatagct C/T ctga c/g cctctcatcttagat | 6188 | | |
| UGT2B15 | 7 | (intron 3 129) | ctaattatctcagacatgtg T/C tcaaa g/a caaaaacatatatg | 6189 | | |
| UGT2B15 | 8 | (intron 3 424) | caataacaataagcaggtat T/C gaaaaaactttgaaatgcat | 6190 | | |
| UGT2B15 | 9 | (intron 3 493) | ggc t/a gtttttacttcccatg C/T attggaataggtctatttag | 6191 | | |
| UGT2B15 | 10 | (intron 3 906) | gccctctctgaatgatctat G/A caagtccttgctgaaaacac | 6192 | | |
| UGT2B15 | 11 | (intron 3 1036) | tcagtaccttagtttggtac T/C agacatggtaatgactggct | 6193 | | |
| UGT2B15 | 12 | (intron 3 1544) | aataaatatataggttatta C/G taatttgctacttttttatt | 6194 | | |
| UGT2B15 | 13 | (intron 3 5550) | gtgtggtgaatcaatgtgtg C/T tgcttgtgggcagtactcca | 6195 | | |
| UGT2B15 | 14 | (intron 3 5720) | ttttttaaaagttaattttt C/A ttggggatttccctgcaggg | 6196 | | |
| UGT2B15 | 15 | (intron 4 134) | atcaaatttaactactttat A/G tttattttccagtcttagta | 6197 | | |
| UGT2B15 | 16 | (intron 5 6627) | ttttaatgttgatatcttta T/C atttatccttcagctataaa | 6198 | | |
| UGT2B15 | 17 | (coding region 1568 (Lys23 | tttccgaaagcttgccaaaa A/C aggaaagaagaagaaaagag | 6199 | | |
| UGT2B15 | 18 | (3′untranslated region 1 | ggatttaatacgtactttag C/T tggaattattctatgtc a/t at | 6200 | | |
| UGT2B15 | 19 | (3′untranslated region 1 | ag c/t tggaattattctatgtc A/T atgatttttaagctatgaaa | 6201 | | |
| UGT2B15 | 20 | (intron 2 1980-1981) | aagagagtagcagaataagg (AGG) acaagggataaatgactagt | 6202 | | |
| UGT2B15 | 20 | (intron 2 1980-1981) | aagagaataacaaaataaga acaaggaataaataactc | 6203 | | |
| UGT2B15 | 21 | (intron 3 605-618) | ctagccaagtagatttagag (A)11-15 cttgtctgctctgctgactt | 6204 | | |
| UGT2B15 | 22 | (3′untranslated region | aagtataatttaaaaaaagc (A)11-14 tacaactcttttttttaaac | 6205 | | |
| | | 1957-1968) | | | | |
| UGT8 | 1 | (coding region 677)Pro226 | gcagaagtacaacctgctgc C/T ggagaagtccatgtatgatt | 6206 | | |
| UGT8 | 2 | (coding region 741)Ala247 | atgctgtgtactgacgtagc A/G ctggaattcccaagacccac | 6207 | | |
| UGT8 | 3 | (intron 2 53-54) | ttgacaatcaataictcctt GT/Δ ttagtgcacaggtcccagta | 6208 | | |
| GSTA1 | 1 | (5′flanking region − 266) | ttgcaaaaagagcaaaatct C/A ggtgaaatgtattgtgtaaa | 6209 | | |
| GSTA1 | 2 | (intron 2 1220) | gagacacaggctttcctaag A/C tatgacaacaccataactag | 6210 | | |
| GSTA1 | 3 | (intron 4 1813) | aaaggcacccactggaggtg A/C attattttgccatcacctga | 6211 | | |
| GSTA1 | 4 | (intron 5 732) | gaagagtgttgtcatgaagg T/C ggagtcactgcccaagggag | 6212 | | |
| GSTA1 | 5 | (intron 6 333) | ttatcccatatgtgcccaca A/G tgagccggtctgagcagagc | 6213 | | |
| GSTA1 | 6 | (3′flanking region 412) | ctttcttatgcatttgcaaa A/C caatgattctgtctgctgtg | 6214 | | |
| GSTA4 | 1 | (intron 1 280) | gcattggtggaaggtgggct C/T ggatcgtccccgggcctggc | 6215 | | |
| GSTA4 | 2 | (intron 3 176) | ggaaatcacttcttattcaa T/C agttccataaaagctggccg | 6216 | | |
| GSTA4 | 3 | (intron 4 94) | acaccacatttactttatgt C/G ttacatagttagtgagatca | 6217 | | |
| GSTA4 | 4 | (intron 5 1062) | cacacttgtgcacatgcaga C/T acccatgggcatccaagagt | 6218 | | |
| GSTA4 | 5 | (coding region 487 | cagatgtgattttactccaa A/G ccattttagctctagaagag | 6219 | | |
| | | (Thr 163 Ala)) | | | | |
| GSTA4 | 6 | (intron 6 595) | tgagctctgagagcaaatga G/A agatgtt a/g gcaccctaaaca | 6220 | | |
| GSTA4 | 7 | (intron 6 630) | taaacatcaccccaaaggat T/A cctaccattctccttctgag | 6221 | | |
| GSTA4 | 8 | (intron 6 3943) | tcttcgtagtatctaatacc T/C tttttgttagccttaaagtt | 6222 | | |
| GSTA4 | 9 | (3′untranslated region 10) | taataacaaccgaatgtcta G/A taaatgactctcctctgagc | 6223 | | |
| GSTA4 | 10 | (intron 5 370-371) | gttgtcgaacagctgtctca (TA) gctgacatcctccctgataa | 6224 | | |
| GSTA4 | 10 | (intron 5 370-371) | gttgtcgaacagctgtctca gctgacatcctccctgataa | 6225 | | |
| GSTM1 | 1 | (5′flanking region − 694) | tacgaagtggctaatttaca C/T agtacttagccagatgaccg | 6226 | | |
| GSTM1 | 2 | (5′flanking region − 661) | gatgaccgaaggactcagta C/T ccgagggcccctaacagaaa | 6227 | | |
| GSTM1 | 3 | (5′flanking region − 658) | gaccgaaggactcagtaccc G/A agggcccctaacagaaaaca | 6228 | | |
| GSTM1 | 4 | (5′flanking region − 656) | ccgaaggactcagtacccga G/A ggcccctaacagaaaacaca | 6229 | | |
| GSTM1 | 5 | (5′flanking region − 537) | tagaggggagactaagccct G/C ggagtagctttcggatcaga | 6230 | | |
| GSTM1 | 6 | (5′flanking region − 525) | taagccctgggagtactgtt C/G ggatcagaggaagtcctgct | 6231 | | |
| GSTM1 | 7 | (5′flanking region − 465) | aattaaattcccaggttggg G/A ccaccactttttagtctgac | 6232 | | |
| GSTM1 | 8 | (5′flanking region − 383) | gcggagcgaaggctgaggga C/T accgcgggcagggaggagaa | 6233 | | |
| GSTM1 | 9 | (5′flanking region − 382) | cggagagaaggctgagggac A/T ccgcgggcagggaggagaag | 6234 | | |
| GSTM1 | 10 | (5′flanking region − 378) | gagaaggctgagggacaccg C/T gggcagggaggagaagggag | 6235 | | |
| GSTM1 | 11 | (5′flanking region − 343) | agggagaagagctttgctcc G/A ttaggatctggctggtgtct | 6236 | | |
| GSTM1 | 12 | (intron 2 118) | tgctggagctgcagctggtc T/C cttccctgagcccgggtgag | 6237 | | |
| GSTM1 | 13 | (intron 3 233) | agtgagtgcccggtctcctc T/C ctgctcttgcttatgggaag | 6238 | | |
| GSTM1 | 14 | (intron 4 26) | tgtgggtggctgcaatgtgt G/A gggggaaggtggcctcctcc | 6239 | | |
| GSTM1 | 15 | (intron 5 140) | actatcagcagttattctca C/T gactccaatgtcatgtcaac | 6240 | | |
| GSTM1 | 16 | (intron 5 577) | ctgccaccccattagaagga A/G ctttctactttccctgagct | 6241 | | |
| GSTM1 | 17 | (intron 5 645) | gctggtctggatccagaggc T/A gccaggtgcttgggcgctcc | 6242 | | |
| GSTM1 | 18 | (coding region 519 | caccgtatatttgagcccaa G/C tgcttggacgccttcccaaa | 6243 | | |
| | | (Asn 173 Lys)) | | | | |
| GSTM1 | 19 | (coding region 528 | tttgagcccaagtgcttgga C/T gccttcccaaatctgaagga | 6244 | | |
| | | (Asp 176 Asp)) | | | | |
| GSTM1 | 20 | (intron 7 2421) | cagcaccgtgtagaatcttc A/G taagtgttagctgttactgt | 6245 | | |
| GSTM1 | 21 | (3′flanking region 42) | atttgctcctggccatctac C/T cagactgtctgtctgtctgt | 6246 | | |
| GSTM2 | 1 | (intron 1 7) | ggaacatccgcggggtgagc C/G agggtccgctgggcggtggg | 6247 | | |
| GSTM2 | 2 | (intron 1 45) | gggacgggggtgcgtggggg C/T ggggaagtgtggagcagctg | 6248 | | |
| GSTM2 | 3 | (intron 3 70) | gactgcatctcctctcccca G/C cttagaggtgttaagatcag | 6249 | | |
| GSTM2 | 4 | (intron 3 224) | agcaggccctggtctcctct T/C tgcccttgcatatgggaagg | 6250 | | |
| GSTM2 | 5 | (intron 5 100) | ttgattccttctggtgagtt C/A ttggtcttgctgactctaag | 6251 | | |
| GSTM2 | 6 | (intron 5 341) | tcctcttggtgggttcatgg T/C ctggctggcttcaggagtga | 6252 | | |
| GSTM2 | 7 | (intron 5 696) | acctttagctagacacagag C/T gctgatttgtgcatttacaa | 6253 | | |
| GSTM2 | 8 | (intron 5 723) | ttgtgcatttacaatccttt A/G gctaggcagaaaagttctcc | 6254 | | |
| GSTM2 | 9 | (3′untranslated region 10 | ctcagccccgagctgtcccc G/A tgttgcatgaaggagcagca | 6255 | | |
| GSTM2 | 10 | (3′flanking region 139) | ttctgctgggcatagtaagg C/T gcttgagaattcttgctccc | 6256 | | |
| GSTZ1 | 1 | (5′flanking region − 546) | agcagggcccaccagccgac C/A gcctcgaagcgccgtgagcc | 6257 | | |
| GSTM2 | 2 | (5′flanking region − 321) | tgtctgaccagccgccccgc T/C aaggagtcacaagagggcag | 6258 | | |
| GSTM2 | 3 | (intron 1 2890) | aaaatactgcatcaaaacca G/A gccacgctctgttgggggga | 6259 | | |
| GSTM2 | 4 | (intron 1 2896) | ctgcatcaaaaccaggccac G/A ctctgttggggggacaccaa | 6260 | | |
| GSTM2 | 5 | (intron 2 255) | tctcccaacactgctctcca A/G agccccttggcaaccatgtt | 6261 | | |
| GSTM2 | 6 | (intron 2 1560) | caccactgtttaaggccgtg G/C gggggcagagttaaacacaa | 6262 | | |
| GSTM2 | 7 | (coding region 94 (Lys 32 | ccttgaaaggcatcgactac G/A agacggtgcccatcaatctc | 6263 | | |
| GSTM2 | 8 | (intron 4 297) | agaaggaggagtttgctggc C/T ctgtcccctctggtccaggg | 6264 | | |
| GSTM2 | 9 | (intron 6 94) | tatctgaaccagccctccag G/A ctgctttgggcctgacagtt | 6265 | | |
| GSTPi | 1 | (intron 1 269) | ctcccccgggctccagcaaa C/G ttttctttgttcgctgcagt | 6266 | | |
| GSTPi | 2 | (intron 2 134) | ccccgggcctccttcgtgtt C/T cccgcctctcccgccatgcc | 6267 | | |
| GSTPi | 3 | (intron 5 438) | gtgtgtgcgcgtgcgtgtgc G/A tgtgtgtgcgtgtgtgtgtg | 6268 | | |
| GSTPi | 4 | (intron 6 162) | cccgctggctgagtccctag C/T ccccctgccctgcagatctc | 6269 | | |
| GSTT1 | 5 | (5′flanking region − 103) | taaagagtgtcccaggcgtc C/T gtgcagcccaatggggcaca | 6270 | | |
| MGST1 | 1 | (promoter region − 1879) | ttaataaatgtttattcaat T/G aaaccaactgctaatattct | 6271 | | |
| MGST1 | 2 | (promoter region − 508) | tctggaccctgaacaggagg G/C gacatcgtgacaaagcaaat | 6272 | | |
| MGST1 | 3 | (promoter region − 314) | cctggagattttaactttct G/A cgaagtttttaaaaacaact | 6273 | | |
| MGST1 | 4 | (promoter region − 131) | atcagcaggcgatccttact G/C tgggcgggtaaatcaggtga | 6274 | | |
| MGST1 | 5 | (intron 1b 36) | ggagaaggggaccgcatgca A/G agggtggcaggcagggaggg | 6275 | | |
| MGST1 | 6 | (intron 1c 456) | ccccttgggacggttctcac C/T tgtgccccacttccccagtc | 6276 | | |
| MGST1 | 7 | (intron 1c 719) | gcccgcaagcattgctgtat A/G gcacccaggcctccagtgag | 6277 | | |
| MGST1 | 8 | (intron 1c 985) | cgagtaaaatttttctaccg C/G tttgttttagagtggtgtct | 6278 | | |
| MGST1 | 9 | (intron 1c 1428) | gtaaagggaaagggcgttcc T/A caactgagaagtgaagattc | 6279 | | |
| MGST1 | 10 | (intron 1c 2914) | ctcatcaggtgtgtgtcgga T/G gcttggtgctggccagtctc | 6280 | | |
| MGST1 | 11 | (intron 1c 4274) | attgtaatagattatcaaag G/T tgatgaaagtagtgtacata | 6281 | | |
| MGST1 | 12 | (intron 1c 4276) | tgtaattgattaacaaaggt G/T atgaaagtagtgtacataat | 6282 | | |
| MGST1 | 13 | (intron 1c 4767) | gccttcatcttcagcacatt C/T ccaattatacttccaattcc | 6283 | | |
| MGST1 | 14 | (intron 2 2379) | ttctcaaatttcattataca G/C tattcttcaacccaaagttt | 6284 | | |
| MGST1 | 15 | (intron 2 2767) | tttaactatagatgccttct T/G ctcctcttgtgtttgattta | 6285 | | |
| MGST1 | 16 | (intron 2 2974) | tcactgcagcctcaacctct C/T gggctcaggtgatcctccaa | 6286 | | |
| MGST1 | 17 | (intron 2 3083) | aaaaaatttgtagatatggg T/G actccctatgttgcccaggc | 6287 | | |
| MGST1 | 18 | (intron 2 3106) | tccctatgttgcccaggctg A/G tcttgaattcttgggctcaa | 6288 | | |
| MGST1 | 19 | (intron 3 1495) | gtcagacaatggccttcagc G/A tcctctctttgcagaatatg | 6289 | | |
| MGST1 | 20 | (intron 3 1703) | ttctcttctaagaagaagtc T/C gtgcagatacttagcacaaa | 6290 | | |
| MGST1 | 21 | (intron 3 2528) | ttttggagacacttttcaga G/C agagcgtttccagcatcttc | 6291 | | |
| MGST1 | 22 | (intron 3 2557) | tccagcatcttccctttcca T/C ttttaagttagacttttttt | 6292 | | |
| MGST1 | 23 | (intron 3 2731) | atacacatatggaacaatta A/C ctaaaaacttaaggtaatat | 6293 | | |
| MGST1 | 24 | (intron 3 3032) | agagacatttagaatatatt C/A cctttaaaggtagagaataa | 6294 | | |
| MGST1 | 25 | (intron 3 3045) | atatattccctttaaaggta G/C agaataacccttcactgaga | 6295 | | |
| MGST1 | 26 | (intron 3 3289) | ggtttatagtgttccccccc T/A ccgggcccccaaaagaccca | 6296 | | |
| MGST1 | 27 | (intron 3 3976) | ggaaagctggggaactgttt G/T cctggaacagagtctcaaaa | 6297 | | |
| MGST1 | 28 | (intron 3 4288) | ccattctatttgtcaactgc G/A taacacaggcgtagaagtgg | 6298 | | |
| MGST1 | 29 | (intron 3 4298) | tgtcaactgcgtaacacagg C/T gtagaagtggacattgtttt | 6299 | | |
| MGST1 | 30 | (intron 3 4429) | attggaggtgacgatatctc T/C gtgatgctgggggagaaatc | 6300 | | |
| MGST1 | 31 | (intron 3 4519) | tttaatagaaaatggtattc C/T tgtcttttctttcccatctc | 6301 | | |
| MGST1 | 32 | (intron 3 4817) | attgctatagaagagagtaa C/T gtaaagcagaaatagttttc | 6302 | | |
| MGST1 | 33 | (intron 3 6077) | tttgaaattagtgtctttaa T/C agttatctttttccacagag | 6303 | | |
| MGST1 | 34 | (3′untranslated region 60 | gggtaaacccattttgaata T/C tagcattgccaatatcctgt | 6304 | | |
| MGST1 | 35 | (3′flanking region 147) | tatttgctttccttctctct C/T tgttttctttttctctgaaa | 6305 | | |
| MGST1 | 36 | (3′flanking region 237) | cagcacgtttttcctatgaa C/T aagacattctccaaataact | 6306 | | |
| MGST1 | 37 | (intron 1c 904-923) | tgcgattatctttggtaatt (A)16-19 ggcaaatcagtccaaatttg | 6307 | | |
| MGST1 | 38 | (intron 1c 3433-3434) | ccccttcaatactagaacaa (AA) gcagacacattaaatgttac | 6308 | | |
| MGST1 | 38 | (intron 1c 3433-3434) | ccccttcaatactagaacaa gcagacacattaaatgttac | 6309 | | |
| MGST1 | 39 | (intron 1c 5146) | actatttcaatttttttttt T/Δ ggagggggagacagagtctc | 6310 | | |
| MGST1 | 40 | (intron 2 552-563) | cccagcattataagaatgac (T)10-12 aagtgcagatgtggggaggg | 6311 | | |
| MGST1 | 41 | (exon 3 172-173) | tagcatttggcaaaggagaa AA/Δ tgccaagaagtatcttcgaa | 6312 | | |
| MGST1 | 42 | (intron 3 152-158) | agaaaactggatgtctgaaa TTGACA/Δ(GTCCAATAT) | 6313 | | |
| | | | cactgcacttgtatgtgttg | | | |
| MGST1 | 43 | (intron 3 2198-2200) | ggattttagattcctcccta CTA/Δ ttctttccgaccttccaccc | 6314 | | |
| MGST1 | 44 | (intron 3 2567-2568) | ccctttccatttttaagtta (A) gacttttttttttcacctct | 6315 | | |
| MGST1 | 44 | (intron 3 2567-2568) | ccctttccatttttaagtta gacttttttttttcacctct | 6316 | | |
| MGST1 | 45 | (intron 3 2571-2580) | tttccatttttaagttagac (T)9-11 cacctctctcgttacttcag | 6317 | | |
| MGST1 | 46 | (intron 3 3288-3289) | ggtttatagtgttccccccc (C) tccccgcccccaaaagaccc | 6318 | | |
| MGST1 | 46 | (intron 3 3288-3289) | ggtttatagtgttccccccc tccccgcccccaaaagaccc | 6319 | | |
| MGST1 | 47 | (intron 3 4682-4683) | tcctcttcatgtctctatgt (GAGATGTTGTGGCTCACAT) | 6320 | | |
| | | | tcctcttcatgtctctatgt | | 6321 | |
| MGST1 | 47 | (intron 4682-4683) | agtcatcctctttgtgagac | | | |
| MGST1 | 48 | (3′flanking region 1359-1 | acacacacacacacacacac CC/Δ tgctctggagttgggcaact | 6322 | | |
| MGST1 | 49 | (3′flanking region 1899-1 | ttagaatagtttctaactat ACT/Δ tttactcccaagagaagctt | 6323 | | |
| MGST1L1 | 1 | (5′flanking region − 105) | tgctgccgctgccgtggggc G/A gggcgtgggcggtgctggct | 6324 | | |
| MGST1L1 | 2 | (intron 1 277) | agtgtctgtgagagaagcag G/A ttctggagggtggagtgtgg | 6325 | | |
| MGST1L1 | 3 | (intron 2 8030) | ggggttatacagagcccctc C/G gcccccaccacacatatgca | 6326 | | |
| MGST1L1 | 4 | (intron 2 8499) | gtatggcaggagtggggtcc C/T ggcaagccatagaggtatgg | 6327 | | |
| MGST1L1 | 5 | (3′untranslated region 46 | cgccacctgtgaccagcagc T/G gatgcctccttggccaccag | 6328 | | |
| MGST2 | 1 | (5′flanking region − 46) | ggtcagcattcaaagtcaag A/T agcgccatttatcttcccgt | 6329 | | |
| MGST2 | 2 | (intron 1 176) | ggtcacccatgccgcctgct A/C ccctccttcccaggggcaag | 6330 | | |
| MGST2 | 3 | (intron 1 204) | tcccaggggcaagcagagac T/C gagaacattccagagattag | 6331 | | |
| MGST2 | 4 | (intron 1 373) | ttacaagtgttccaaaggaa A/T cgtgcctgcttctaaacctg | 6332 | | |
| MGST2 | 5 | (intron 2 − 3245) | cctcgtgatttgcccacctc G/A gcctcccaaagtgctgggat | 6333 | | |
| MGST2 | 6 | (intron 2 − 1998) | aggccgaggtgggcggatca T/C gaggtcaggagatcgagacc | 6334 | | |
| MGST2 | 7 | (intron 2 − 1640) | tgtttattccttgcatagcc A/G taatataaagtatgaatttt | 6335 | | |
| MGST2 | 8 | (intron 3 41) | actgtgttctaatgatgact A/G tgatgcttaaacgattaagg | 6336 | | |
| MGST2 | 9 | (intron 3 453) | atcagagtgctatgttgcag A/G tatatgaactttggcttcat | 6337 | | |
| MGST3 | 1 | (5′flanking region − 520) | acaaaaaggccctaacagcg A/C taaatccattcacttcggga | 6338 | | |
| MGST3 | 2 | (5′flanking region − 355) | cgcctaaaaccgctacggtg G/A ctctgctggggacaaattat | 6339 | | |
| MGST3 | 3 | (5′flanking region − 234) | ctgggggagtagatatatgt T/A tttgagaatgagaggagtaa | 6340 | | |
| MGST3 | 4 | (intron 1 74) | agcctttgcgcaggcacctc C/T atatttcagcctatgcgagc | 6341 | | |
| MGST3 | 5 | (intron 1 682) | agaaaatgccccttctttat G/C tggggtggcagcacggagcc | 6342 | | |
| MGST3 | 6 | (intron 1 832) | cgagtttacaagctacataa T/C agcgtcgggggcaagtaagt | 6343 | | |
| MGST3 | 7 | (intron 1 1919) | aataaaattcctgagtttct G/C tcactcgctcttacagtacc | 6344 | | |
| MGST3 | 8 | (intron 1 1991) | tgtaattaggcaacaggaaa A/G ttgtactatctttcaaatgc | 6345 | | |
| MGST3 | 9 | (intron 1 4458) | tcttccatcctcctaacata T/C agttagcttccactctccaa | 6346 | | |
| MGST3 | 10 | (intron 1 4676) | tgaatatgcaatgcaattgt C/G gggggatagttacttttcat | 6347 | | |
| MGST3 | 11 | (intron 3 278) | cagcataacccatctaaacc G/C atgttgactctcccacccct | 6348 | | |
| MGST3 | 12 | (intron 4 423) | cttgcctttttgttgtgggg T/G gtggggtggtcacagaaaag | 6349 | | |
| MGST3 | 13 | (intron 4 506) | gtgcagagaagaaaacaaag T/C ggggaaggtggaaaggggat | 6350 | | |
| MGST3 | 14 | (intron 4 − 162) | tcacagatattttattttcc C/T gactgaaactaacttaattc | 6351 | | |
| MGST3 | 15 | (intron 4 − 130) | acttaattctacctaatttg C/G gtggggagtagttggccaaa | 6352 | | |
| MGST3 | 16 | (intron 4 − 105) | ggagtagitggccaaatcat C/G aaattgttaactttttgcta | 6353 | | |
| MGST3 | 17 | (intron 4 − 65) | aacatattgtgtaatcaacc C/T taggtgttaaaaaaggtttg | 6354 | | |
| MGST3 | 18 | (intron 5 105) | atcccagcactttgggaggc G/C aaggcaggcagattgcttga | 6355 | | |
| MGST3 | 19 | (intron 5 197) | aaaaaatacaaaaattagcc G/A gatgtggtagtgcacacctg | 6356 | | |
| MGST3 | 20 | (intron 5 222) | tggtggtgcacacctgtagt C/T ccagctacttgggaggctga | 6357 | | |
| MGST3 | 21 | (intron 5 374) | cttatgctactatatttttt T/C ttcttgggaatttgagaaaa | 6358 | | |
| MGST3 | 22 | (3′untranslated region 51 | atgacttacctttatttcca G/T ttacattttttttctaaata | 6359 | | |
| MGST3 | 23 | (3′flanking region 166) | agtctgattgtggtgatgta G/T gtatagtcatgccacagtga | 6360 | | |
| SULT1A1/ST | 1 | (5′flanking region − 1597) | gcagagtaaagggactcact C/G aagaagaggaacgtgggggt | 6361 | | |
| SULT1A1/ST | 2 | (5′flanking region − 1491) | gagaggtatattcatgaaga G/T tccaggaaaaggtaaagatt | 6362 | | |
| SULT1A1/ST | 3 | (5′flanking region − 1376) | cggtttcatatgttactgat C/T a/a taca a/g tgaaatcctaagt | 6363 | | |
| SULT1A1/ST | 4 | (5′flanking region − 1375) | ggtttcatatgttactgat c/t A/G taca a/g tgagatcctagctc | 6364 | | |
| SULT1A1/ST | 5 | (5′flanking region − 1370) | catatgttactgat c/t a/g taca A/G taagatcctaggtaaaac | 6365 | | |
| SULT1A1/ST | 6 | (exon 1B − 65) | aaccctgcattccccacaca G/A cacccacaatcagccactgc | 6366 | | |
| SULT1A1/ST | 7 | (intron 1B 442) | gagccaccctgcctaggcct G/A tgcttttgctgagtcatcag | 6367 | | |
| SULT1A1/ST | 8 | (exon 1A − 197) | gctgggggtcccagcaggaa A/G tggtgagacaaagggcgctg | 6368 | | |
| SULT1A1/ST | 9 | (exon 1A − 159) | ctggctggcagggagacagc A/C caggaaggtcctagagcttc | 6369 | | |
| SULT1A1/ST | 10 | (exon 1A − 95) | gagaccttcacacaccctga T/C atctgggccttgcccgacga | 6370 | | |
| SULT1A1/ST | 11 | (intron 1A 60) | ctggttttcagccccagccc C/T gccactga c/g tggctttgtga 6371 | | | |
| SULT1A1/ST | 12 | (intron 1A 69) | agccccagccc c/t gccactga C/G tggctttgtgagtgcgggca 6372 | | | |
| SULT1A1/ST | 13 | (intron 1A 174) | tgtgatggtggtaagggaac G/A ggcctggctctggcccctga | 6373 | | |
| SULT1A1/ST | 14 | (intron 6 11) | catgaaggaggtgagaccac C/G igtga a/t gcttccctccatgt 6374 | | | |
| SULT1A1/ST | 15 | (intron 6 17) | ggaggtgagaccac c/g tgtga A/T gcttccctccatgtgacacc 6375 | | | |
| SULT1A1/ST | 16 | (intron 6 35) | gaagcttccctccatgtgac A/T cctgggggccggcacctcac | 6376 | | |
| SULT1A1/ST | 17 | (intron 6 71) | ctcacagggacccaccaggg T/C cacccagccccctcccttgg | 6377 | | |
| SULT1A1/ST | 18 | (intron 6 100) | ttggcagcccccacagcagg C/A cc g/a gattccccatcctgcct 6378 | | | |
| SULT1A1/ST | 19 | (intron 6 111) | gcagcccccacagcagg c/a cc G/A gattccccatcctgccttct 6379 | | | |
| SULT1A1/ST | 20 | (intron 6 270) | ctccctgccaaaaggtgtgc C/T acccagggccacagtcatgg | 6380 | | |
| SULT1A1/ST | 21 | (intron 6 488) | ttttacttttcctgaatcag C/T aatccgagcctccactaagg | 6381 | | |
| SULT1A1/ST | 22 | (intron 6 509) | aatccgagcctccactgagg A/G gccctctgctgctcagaacc | 6382 | | |
| SULT1A1/ST | 23 | (coding region 600 | ccctctgctgctcaaaaccc C/G aaaagggagattcaaaagat | 6383 | | |
| | | (Pro 201 Pro)) | | | | |
| SULT1A1/ST | 24 | (coding region 638 | gatcctggagtttgtgggac A/G ctccctgccagaggagaccg | 6384 | | |
| | | (His 213 Arg)) | | | | |
| SULT1A1/ST | 25 | (coding region 645 | gagtttgtggggcactccct G/A ccagaggaaaccgtggactt | 6385 | | |
| | | (Leu 215 Leu)) | | | | |
| SULT1A1/ST | 26 | (coding region 902 | gctgtgagaggagctcctgg G/A gtcactgcagagggagtgtg | 6386 | | |
| | | (Gly 301 Ser)) | | | | |
| SULT1A1/ST | 27 | (coding region 973 | taaaatatgaattgagggcc T/C gggacggtaggtcatgtctg | 6387 | | |
| | | (Trp 325 Arg)) | | | | |
| SULT1A2/ST | 1 | (5′flanking region − 547) | tgttctttcttggttctatg G/C atccatgctctgctccaccc | 6388 | | |
| SULT1A2/ST | 2 | (5′flanking region − 425) | tgtgggttgcactgggccag G/A acccctggcaccttcaaaac | 6389 | | |
| SULT1A2/ST | 3 | (5′flanking region − 358) | ctttccagggcctgcctatc C/T ca g/t ctttctcctccaatccc | 6390 | | |
| SULT1A2/ST | 4 | (5′flanking region − 355) | tccagggcctgcctatc c/t ca G/T ctttctcctccaatccctcc | 6391 | | |
| SULT1A2/ST | 5 | (5′untranslated region − | actgcgggcgaggagggcac A/G aggccaggttcccaagaact | 6392 | | |
| SULT1A2/ST | 6 | (intron 1A 85) | ctgactggccttgtgagtgc G/A ggcaagtcactcagcctccc | 6393 | | |
| SULT1A2/ST | 7 | (coding region 20 | catggagctgatccaggcca T/C ctc t/c cgcccgccactggagt | 6394 | | |
| SULT1A2/ST | 8 | (coding region 24 (Ser 8 S | gagctgatccaggaca t/c ctc T/C cgcccgccactggagtacgt | 6395 | | |
| SULT1A2/ST | 9 | (intron 2 34) | gccacccaccctctcccagg T/C ggcagtccccaccttggcca | 6396 | | |
| SULT1A2/ST | 10 | (intron 5 77) | cagcaaccctgtgtcggcac T/C ccctgcccacttctccagtg | 6397 | | |
| SULT1A2/ST | 11 | (intron 6 684) | actggggtcccaggggtcga G/C gagctggctctatgggtttt | 6398 | | |
| SULT1A2/ST | 12 | (coding region 704 | gttcaaggagatgaagaaga A/C ccctatgaccaactacacca | 6399 | | |
| | | (Asn 235 Thr)) | | | | |
| SULT1A2/ST | 13 | (3′untranslated region 89 | gctctgagctgtgagagggg T/C tcctggagtcactgcagacc | 6400 | | |
| SULT1A2/ST | 14 | (3′flanking region 98) | cctccccgctccagctcctc A/T acttgccctgtttggagagg | 6401 | | |
| SULT1A2/ST | 15 | (3′flanking region 817) | ccactgactcccggcttgcc A/C aggctgccagggctggcaaa | 6402 | | |
| SULT1A2/ST | 16 | (3′flanking region 1006) | cctctcccctggaggctgct T/C tacccgctgtgggggcgcat | 6403 | | |
| SULT1A2/ST | 17 | (3′flanking region 1464) | tcccgtagcccaggcaagtt C/T ggtgaccagagagcagcccc | 6404 | | |
| SULT1A2/ST | 18 | (intron 4 1728) | tcagcttcctcctttgccaa A/Δ cccagagatgagctggccta | 6405 | | |
| SULT1A3/ST | 1 | (coding region 843 | cgcttcgatgcggactatgc G/A gagaagatggcagactgcag | 6406 | | |
| | | (Ala 281 Ala)) | | | | |
| SULT1C1 | 1 | (intron 3 2280) | gcaaatttttggtattttta G/T tacagtcagggttttaccat | 6407 | | |
| SULT1C1 | 2 | (intron 3 3742) | gcagatctcactttctggca G/A attccctgaatttgctcccc | 6408 | | |
| SULT1C1 | 3 | (intron 3 4453) | ttcatagggcttttccctca C/T ttgttttgtaattttgtata | 6409 | | |
| SULT1C1 | 4 | (intron 3 5234) | gaaaagcgactagaggcagg A/G gagctttgcagttcttctaa | 6410 | | |
| SULT1C1 | 5 | (intron 3 6175) | tggctggcaggaaggtgagg G/C agtcctctcttctctggtcc | 6411 | | |
| SULT1C1 | 6 | (intron 4 205) | acatgaaggcaggatccaga T/C tgaatgtttggagggaacta | 6412 | | |
| SULT1C1 | 7 | (intron 4 408) | ggctcacgcctgtaatccca G/C cactttgggaggccgaggcg | 6413 | | |
| SULT1C1 | 8 | (intron 4 429) | cactttgggaggccgaggcg G/C gtggatcacaaagtcaggag | 6414 | | |
| SULT1C1 | 9 | (intron 3 2106-2115) | tgcagtggtcgtttgtttgg (T)8-11 gagacaaagtgtggctgtgt | 6415 | | |
| SULT1C1 | 10 | (intron'4195-4210) | atgcccagctaattttggta (T)10-13 agagacaggatttcaccatg | 6416 | | |
| SULT1C2 | 1 | (5′flanking region − 110) | tcctgttaactcacagagaa C/T ggaagggctggaacgggacc | 6417 | | |
| SULT1C2 | 2 | (coding region 15 (Asp 5 G | acactaatggccttacacaa C/G atggaggattttacatttga | 6418 | | |
| SULT1C2 | 3 | (intron 1 297) | gtagacttgtttatttattc A/C ttcccaatctaggcccttat | 6419 | | |
| SULT1C2 | 4 | (intron 1 363) | gagtgtgtgagctagacagg T/G gatcctgagtctgatttggg | 6420 | | |
| SULT1C2 | 5 | (intron 1 2300) | gggctactatgagcagccac C/T acctcaggaaggatgacttc | 6421 | | |
| SULT1C2 | 6 | (intron 2 455) | aagacttggaagcaaataga T/G aaaaaaaaaatcgtagaaat | 6422 | | |
| SULT1C2 | 7 | (intron 4 55) | caaaatctccaaacaggcta G/A aaggaaagaatcttttcttt | 6423 | | |
| SULT1C2 | 8 | (intron 4 111) | ctgccttctttaatggaaca T/C tctcacttctcttcaggaat | 6424 | | |
| SULT1C2 | 9 | (intron 5 1657) | ctttgtgtttactttgtttt T/C acttggtacaaaagtgttgt | 6425 | | |
| SULT1C2 | 10 | (intron 5 2082) | tctgctcctagagatggagg c/A gtcccacagccacagtgatg | 6426 | | |
| SULT1C2 | 11 | (intron 6 933) | agctagtgaacccctcccac A/G taactgtatttcaggggcag | 6427 | | |
| SULT2A1 | 1 | (intron 2 478) | ggactggctgctgtacacac T/C tcgtcttactgtgtgtaaat | 6428 | | |
| SULT2A1 | 2 | (intron 3 382) | caaaaccctcttaatattct G/A tttctatctgtctcagaact | 6429 | | |
| SULT2A1 | 3 | (intron 3 409) | tctgtctcagaactgattgc A/G tgactctaggatcgctatat | 6430 | | |
| SULT2A1 | 4 | (intron 5 249) | agctggaaattacaggcaca C/T gccaccacacccagctaatt | 6431 | | |
| SULT2A1 | 5 | (intron 5 395) | aggcatgagccagggcgccg G/A gccaatttatcaactttaat | 6432 | | |
| SULT2A1 | 6 | (3′flanking region 33) | ttccttgttaaaagtcacca G/C ggttggccaggc a/g cggtggt | 6433 | | |
| SULT2A1 | 7 | (3′flanking region 46) | gctacca g/c ggttggccaggc A/G cagtggttcaggcccccaac | 6434 | | |
| SULT2A1 | 8 | (3′flanking region 199) | ttagccaggcgcattggctc A/G tgtctgtaatcccagcactt | 6435 | | |
| SULT2B1 | 1 | (intron 2 4162) | ctctccccgcggcctaccat C/T cgcacacaggtgatctacat | 6436 | | |
| SULT2B1 | 2 | (intron 3 879) | gagggcatccagctctgggg G/A ctggacctgggggtttgtgg | 6437 | | |
| SULT2B1 | 3 | (intron 4 3882) | ttccacgctccttccggggc C/T gagcgccccccccccgccga | 6438 | | |
| SULT2B1 | 4 | (intron 5 1780) | cctgcagaagggggtccctt C/T catgtccaaacagtaatggc | 6439 | | |
| SULT2B1 | 5 | (intron 5 1814) | taatggctgcagcatggagc G/A gtgtggaggcattgagacag | 6440 | | |
| SULT2B1 | 6 | (coding region 789 | ccctcttctccaggggtctg C/T ggcgagtggaagaaccactt | 6441 | | |
| | | (cys 263 cys)) | | | | |
| SULTX3 | 1 | (intron 1 332) | cctgcttctccctttacctg G/T ctgactgtatgaccttggac | 6442 | | |
| SULTX3 | 2 | (intron 1 1167) | caggaatggctaagcgtatc G/A ttggcttctgtggccactca | 6443 | | |
| SULTX3 | 3 | (intron 1 2872) | cattctcactgatgcagagg G/A aagcttctgggcctgggcgt | 6444 | | |
| SULTX3 | 4 | (intron 1 6242) | cacccttggcttttaccagc A/G tggaaacattttacctgaat | 6445 | | |
| SULTX3 | 5 | (intron 1 6601) | gcgtgggcttctggagggag C/T gagaggagagtggagggcgg | 6446 | | |
| SULTX3 | 8 | (intron 1 6768) | agcttgaaatgagccagact C/T ccctgggacctgttgacccc | 6447 | | |
| SULTX3 | 7 | (intron 1 6905) | agtactttgttttatcctcc C/T catcctcacaactttgccat | 6448 | | |
| SULTX3 | 8 | (intron 1 7464) | gccaggatcccttgagagac G/A acatgaacacagccaggagc | 6449 | | |
| SULTX3 | 9 | (intron 1 7833) | tgcttcgggctgggcttggc G/A ggggcagctgtgctccaggc | 6450 | | |
| SULTX3 | 10 | (intron 1 8189) | caaactggggcccttaatgc C/T gcacaccagagcctcctttc | 6451 | | |
| SULTX3 | 11 | (intron 1 8316) | ctctcacacaagggcggagc C/G ccttccccttgaggcagagc | 6452 | | |
| SULTX3 | 12 | (intron 1 8617) | agacagaggctggggccaag C/T cagggttgccggagcttccc | 6453 | | |
| SULTX3 | 13 | (intron 1 8631) | gccaagccagggttgccgga G/T cttcctggactggtcaggcc | 6454 | | |
| SULTX3 | 14 | (intron 1 9493) | ccccccccccagagcggccc G/A ccgtgctctgtgtcgagggc | 6455 | | |
| SULTX3 | 15 | (intron 1 10306) | caggcggggagcctgaatgc C/T gcagtcgtgagggtggccag | 6456 | | |
| SULTX3 | 16 | (intron 1 11987) | ccataaaataatgatatcag T/C acactttttggaaatttgag | 6457 | | |
| SULTX3 | 17 | (intron 1 13085) | ctctgtgcccggtgttgaga C/A aggccatgccctagagtcct | 6458 | | |
| SULTX3 | 18 | (intron 1 13108) | gccatgccctagagtcctgg G/A gagttccaccccagaacagc | 6459 | | |
| SULTX3 | 19 | (intron 2 700) | gaaccatctgggagtcgttc C/T gtactgccgtgccgagggcc | 6460 | | |
| SULTX3 | 20 | (intron 2 818) | agccatagtagctagccagc G/A atcagcgctgggaggggagc | 6461 | | |
| SULTX3 | 21 | (intron 2 1677) | accccacggccccggaaccc C/T accccttccttcctcctctg | 6462 | | |
| SULTX3 | 22 | (intron 4 4954) | gcgtgccgaaggcgggaggg C/T cgggatggctcaagaggtga | 6463 | | |
| SULTX3 | 23 | (intron 5 3632) | ccagcggacccccacaccag C/T ggtcagagaacattgtcttt | 6464 | | |
| SULTX3 | 24 | (intron 5 3662) | acaccgccggccaaggccgc C/T gaagtgctgcaataaagaaa | 6465 | | |
| SULTX3 | 25 | (intron 6 1874) | cctgatctcagagagctgac A/G atggaaagaattctaaacga | 6466 | | |
| SULTX3 | 26 | (intron 6 2133) | agaccggtgcctgcagttta T/G cccacagctcagcccgccct | 6467 | | |
| SULTX3 | 27 | (intron 6 2524) | ggaagggccagggctgcctg T/C gatgcccagagcagtgcact | 6468 | | |
| SULTX3 | 28 | (intron 6 2573) | agatcatactcgctcctggg A/G cgcccaccaaacaccggcca | 6469 | | |
| SULTX3 | 29 | (3′flanking region 12) | gttcccggcgttgcgtcgag C/G gtttctgcttgtgggggtag | 6470 | | |
| SULTX3 | 30 | (3′flanking region 445) | tccaaagcctgtcttcctga T/G ttcctgtggaaggagagtcc | 6471 | | |
| SULTX3 | 31 | (intron 1 6418) | ctctccctgttagtgtgggg G/Δ cagctctttccagtgtcctg | 6472 | | |
| SULTX3 | 32 | (intron 5 2458) | cccttaaagggaagttcatc C/Δ ttctctgccttccaggctcc | 6473 | | |
| TPST1 | 1 | (5′flanking region − 298) | acccgccaccatgnccagct A/C attttttttgtatttttttt | 6474 | | |
| TPST1 | 2 | (intron 1 3520) | agaaaagcagattaatgtaa C/G agtgacgcttagacaacaag | 6475 | | |
| TPST1 | 3 | (intron 1 3610) | ggcagaaagagaatatagca A/G ctattaaacacaaataaatt | 6476 | | |
| TPST1 | 4 | (intron 1 20820) | tattgctgtccacctggtca A/G tgtgtcctctggataagtgc | 6477 | | |
| TPST1 | 5 | (intron 1 − 6761) | aatacaatacttattctgta T/C aattctagagggcccagaga | 6478 | | |
| TPST1 | 6 | (intron 1 − 544) | tagaacaagtgaatatttta C/T gttcttagtggtttatggtt | 6479 | | |
| TPS71 | 7 | (intron 1 − 526) | tacgttcttagtggtttatg G/T ttggcagttttcccccaaca | 6480 | | |
| TPST1 | 8 | (intron 1 − 234) | tcaagacatttaataatgca C/T acgtttcagccaaccctttt | 6481 | | |
| TPST1 | 9 | (intron 1 − 48) | ttatagtgggtttaagcatg A/G tttctaaaaaatttaaataa | 6482 | | |
| TPST1 | 10 | (intron 2 − 18944) | aaaacattagaactgggaag G/A ttaaaaaatctttagtcttt | 6483 | | |
| TPST1 | 11 | (intron 2 − 18687) | tctgtgcaccctaataacat A/G tttccttaaaactagtacta | 6484 | | |
| TPST1 | 12 | (intron 2 − 18501) | ttggaaggtaacttaatgta A/G gtgcctgaaaaacagggata | 6485 | | |
| TPST1 | 13 | (intron 2 − 159) | gaatggggatttccctcagt C/G ctgcccactggctgctcttg | 6486 | | |
| TPST1 | 14 | (intron 2 − 19) | acctgttgccttaaactcac G/A cctgctttgtttttccaggt | 6487 | | |
| TPST1 | 15 | (intron 3 158) | cgctggggaagaaagatcag C/G gtctgggacttgttgatttt | 6488 | | |
| TPST1 | 16 | (intron 3 3779) | agcagggcacgtcaccctcc C/T ggcacacccatgtgttcacc | 6489 | | |
| TPST1 | 17 | (intron 4 292) | ttgttattttcattatgaac C/T atgaaatatttaagctggaa | 6490 | | |
| TPST1 | 18 | (3′untranslated region 15 | gttgtctgtacatgttctaa T/G gttttgtagaacacgtgtgc | 6491 | | |
| TPST1 | 19 | (3′flanking region 264) | acggtgcttggcctgcatta C/T cattttgtagtgaagtttct | 6492 | | |
| TPST2 | 1 | (intron 2 578) | tcacctatcatcctcactgc G/A aggatgccaggatacctccc | 6493 | | |
| TPST2 | 2 | (intron 2 789) | cttaagccatcgtgcaggtc A/G ttgctgtcttctgctcactt | 6494 | | |
| TPST2 | 3 | (intron 3 2009) | cccaggctggagtgtagtgg T/C gtgatct c/t ggctcactgcaa | 6495 | | |
| TPST2 | 4 | (intron 3 2017) | ggagtgtagtgg t/c gtgatct C/T ggctcactgcaacctccgcc | 6496 | | |
| TPST2 | 5 | (intron 3 2035) | ctcggctcactgcaacctcc G/A cctcccgggttcaagcagtt | 6497 | | |
| TPST2 | 6 | (intron 4 104) | aatgttcagtctctcaattc C/T tggtcgtctgatttgttcct | 6498 | | |
| TPST2 | 7 | (intron 4 379) | taaataaataaactattggt C/T cctttcttgtcttataaggt | 6499 | | |
| TPST2 | 8 | (intron 4 588) | tacagcagcctgatacttct C/T ggcttgagccatcctctcac | 6500 | | |
| TPST2 | 9 | (intron 4 626) | caccccaggctcctgagtag C/T taggactgcaggtgcacgcc | 6501 | | |
| TPST2 | 10 | (intron 4 718) | cccaggctggtctagaactc C/G tggccgtaagggatgcccct | 6502 | | |
| TPST2 | 11 | (intron 4 873) | gttgatggccttatttatac G/A tttccattacagcttctagt | 6503 | | |
| TPST2 | 12 | (intron 4 949) | caaatatttgaaaatgggac C/G caggcccgaggaagagcttt | 6504 | | |
| TPST2 | 13 | (intron 4 1033) | taagctcagcatttctgagc G/A tgtgctgattttaggaaata | 6505 | | |
| TPST2 | 14 | (intron 4 1051) | gcgtgtgctgattttaggaa A/G taaacagttatcgtattgaa | 6506 | | |
| TPST2 | 15 | (intron 4 1356) | gattcaacgtacataccagc C/T gacattgacaggtggatggc | 6507 | | |
| TPST2 | 16 | (intron 4 1707) | gtctccttaaaaggtggctc G/T ctgcccctggctcgccccag | 6508 | | |
| TPST2 | 17 | (intron 5 215) | aagaccagcctgaccaaaac G/A gtgaaaccccgtctctacta | 6509 | | |
| TPST2 | 18 | (intron 5 341) | tgggaggcagaggtcgcagt G/A agctgagatcgcgccgttgc | 6510 | | |
| TPST2 | 15 | (intron 6 31) | ggacttcactgggggttccc G/A ctgcttctgggtggecccgg | 6511 | | |
| TPST2 | 28 | (intron 6 273) | gtttgtctgacactggggac A/G gggcaggaagcaccactatg | 6512 | | |
| TPST2 | 21 | (intron 6 693) | aaagggatttttttgaactt G/C gtaattcaaagatttaagat | 6513 | | |
| TPST2 | 22 | (intron 6 1635) | ccctgggtacagagttggcc T/G tgaacaaacatgagtccttc | 6514 | | |
| TPST2 | 23 | (3′untranslated region 11 | cttccccactttcagatctc C/T gcaaatggcttcattgccaa | 6515 | | |
| CST | 1 | (intron 1b 6302) | agagctccccagagaggact A/G tgaggctgcatgatgcatga | 6516 | | |
| CST | 2 | (intron 2a 1004) | gagtgagacccccatctctg C/T aaaattttttttaaaaagta | 6517 | | |
| CST | 3 | (intron 2a 1395) | atgcctaagtttacagtagc T/C aggcaggaaaggcacaacca | 6518 | | |
| CST | 4 | (intron 1d 473) | ccagagcctgaggttggtgc T/A ggggcccctccatggctgcc | 6519 | | |
| CST | 5 | (intron 2b 726) | ctatctctccagtgcctctc T/C gcccctgtctggaccctgct | 6520 | | |
| CST | 6 | (intron 2b 745) | ctgtccctgtctggaccctg C/A tggggggccacagagcaggc | 6521 | | |
| CST | 7 | (coding region 55 | tcactagtttcctgctgctg C/A tgtactcctatgccgtgccc | 6522 | | |
| | | (Val 29 Met)) | | | | |
| CST | 8 | (intron 3 308) | tcgtctgaggtcaggagttc G/A agaccagcctggccaacatg | 6523 | | |
| CST | 9 | (intron 3 853) | ttttgtcctataaaatggca G/A tttcatgtggcccaagctga | 6524 | | |
| CST | 10 | (Asn 66 Asn)) | gaggcagtgatccgggccaa C/T ggctcggcgggggggtgcca | 6525 | | |
| ST1B2 | 1 | (intron 1 80) | acttgtccataaaatcatta C/T cattctaaataaagttaata | 6526 | | |
| ST1B2 | 2 | (intron 2 − 352) | aacatttaaatagtcattta T/C agcaatgcacaggcataata | 6527 | | |
| ST1B2 | 3 | (intron 2 − 85) | attacataatgctaaaaaat G/A tcttgaaaaactggttggca | 6528 | | |
| ST1B2 | 4 | (intron 4 460) | gtacttgacattaaaaaata T/C ctgatgttt a/g tatatccata | 6529 | | |
| ST1B2 | 5 | (intron 4 470) | ttaaaaaata t/c ctgatgttt A/G tatatccataaatagctaat | 6530 | | |
| ST1B2 | 6 | (intron 4 518) | tttaagattgtcctcatatt C/G ttacttcctttggttactaa | 6531 | | |
| ST1B2 | 7 | (intron 4 616) | aatgtttatgaaaatagact T/C ttatctggttttagtggcct | 6532 | | |
| ST1B2 | 8 | (intron 5 58) | ctgcatcatgctgtaaaagg G/A gtgatatttgctttccaact | 6533 | | |
| ST1B2 | 9 | (coding region 612 | taatagaatccaaaggagga A/C atcaagaagatcattagatt | 6534 | | |
| | | (Glu 204 Asp)) | | | | |
| ST1B2 | 10 | (intron 6 582) | aatacattacttccatttaa G/A tagcctgcccatcgcggctt | 6535 | | |
| ST1B2 | 11 | (intron 6 3130) | agatgtaaaaaattattcaa A/T ttttaaaagccggaaaaatt | 6536 | | |
| ST1B2 | 12 | (3′untranslated region 90 | cctaaagtgcctaaaccaca C/A atcggaagaaacaagagatt | 6537 | | |
| ST1B2 | 13 | (3′flanking region 55) | tcagatcccagttttgttcc T/G ttgattctgagtttccaaat | 6538 | | |
| ST1B2 | 14 | (3′flanking region 328) | tttgacccaggacactgtgt T/G ccactgctgtctaccgagtt | 6539 | | |
| ST1B2 | 15 | (3′flanking region 446) | gtagttcagattttggaaat C/A ttttttctatatcataccta | 6540 | | |
| CHST1 | 1 | (intron 1 3900) | gccctgcccccactcccaga C/G ttgcggccctccagcccctt | 6541 | | |
| CHST1 | 2 | (intron 1 6520) | cctcccccagaggagctggg C/T acactggggccttgtgttgt | 6542 | | |
| CHST1 | 3 | (intron 1 7963) | aaaacattcatgggggatta G/C tgctggctacgtcagagtca | 6543 | | |
| CHST1 | 4 | (intron 1 9173) | gcgctgccacagatcaggcc G/A aggtgggggacagaaatgcc | 6544 | | |
| CHST1 | 5 | (intron 1 9701) | cccagaattctgaatacagc A/G gcgatgacgggactacgagg | 6545 | | |
| CHST1 | 6 | (intron 1 12132) | aacagatccacaggaccaga C/A agcaaaggggaggaacatgc | 6546 | | |
| CHST1 | 7 | (intron 1 12465) | atgcagggaaggggcttggc G/A caaaactgtcaactgagata | 6547 | | |
| CHST1 | 8 | (intron 1 12561) | atgctccctggtccactttc G/A ctttgagtttcaggtagctg | 6548 | | |
| CHST1 | 9 | (intron 3 529) | ccatggtctgcaggggtcct T/G catgctcaggggattggggt | 6549 | | |
| CHST1 | 10 | (intron 3 617) | agaggacagaggaaagagga C/A cacctggagaactgggcgcc | 6550 | | |
| CHST1 | 11 | (intron 3 796) | aagaggcttccgcagctgtc C/T gcaggttaaatcctggggtg | 6551 | | |
| CHST1 | 12 | (intron 3 818) | caggttaaatcctggggtgc A/G aggaatgtttgttcagctcc | 6552 | | |
| CHST1 | 13 | (3′flanking region 762) | ataactggtacaggtttact G/C gtgtctacactggcagagaa | 6553 | | |
| CHST1 | 14 | (intron 1 7874) | gttttccccttgccttgcct T/Δ cattttcatcacctcatttt | 6554 | | |
| CHST1 | 15 | (3′flanking region 335-3 | cacactgccacacctggcta (T)12-15 ggattttagtagagacgggg | 6555 | | |
| CHST2 | 1 | (5′flanking region − 260) | agccggacagtccgccgggc G/A gtgatccgggggccgctccc | 6556 | | |
| CHST2 | 2 | (5′flanking region − 56) | gcgctggggaccagcggccg C/T gcccgcctgggagtcgcggc | 6557 | | |
| CHST2 | 3 | (3′flanking region 218) | aggagtgaaacacatctttg T/A attctaaaggcagaaaccaa | 6558 | | |
| CHST2 | 4 | (3′flanking region 383) | gcagagaccaatgttttggt G/C ctgagctgggttcagaaaaa | 6559 | | |
| CHST2 | 5 | (2340 flanking region 952) | tactgaaacattctgcagaa T/C gttatactctgagaagaaat | 6560 | | |
| CHST3 | 1 | (5′untranslated region − 2 | tccagcgtgccgaccggccc C/G gcagcgcctccatccctccg | 6561 | | |
| CHST3 | 2 | (intron 1 96) | gcgtccaggcgcgcgcgcca G/A actttggagggagaaggggg | 6562 | | |
| CHST3 | 3 | (intron 1 4467) | agagaagaatggggcagagc C/G ggagcagccaggggaggtga | 6563 | | |
| CHST3 | 4 | (intron 1 4853) | ggatgagcactgcccagctg A/G cccctgcccaccctccacag | 6564 | | |
| CHST3 | 5 | (intron 1 4965) | cccacggcagaggggacaca G/C tgaccaggacggaagttggg | 6565 | | |
| CHST3 | 6 | (intron 1 5046) | gggcggtccatctttgtacc C/T cgggttccatcccagtgcct | 6566 | | |
| CHST3 | 7 | (intron 1 5300) | ccttttctcctctaaggcct A/G aagagatgacagaatgctgc | 6567 | | |
| CHST3 | 8 | (intron 1 5354) | agcgcgtggactccacagcg G/A ggcgtggggtggcccctggc | 6568 | | |
| CHST3 | 9 | (intron 1 5428) | gacacgcttcagccctctgt C/G cccaccgccccaaatctggc | 6569 | | |
| CHST3 | 10 | (intron 1 6555) | gagtggggcactgctggaag G/C ctctggcccctgcggcgccc | 6570 | | |
| CHST3 | 11 | (intron 1 6990) | aaacacactgggcccccccc G/A tccccgcactgtgactacac | 6571 | | |
| CHST3 | 12 | (intron 1 7133) | ctgagggcctgtcctgcagg T/G ttgatgtgtctgaagaggcc | 6572 | | |
| CHST3 | 13 | (intron 1 7161) | gtctgaagaggccccgagaa T/C agaaatctagaacctgccag | 6573 | | |
| CHST3 | 14 | (intron 1 7199) | cagtcacgaagcagtgtcac C/T caccagaggatgaagaactg | 6574 | | |
| CHST3 | 15 | (intron 1 7316) | cttgcatctggtgtaggtgc C/T cgggggtagcgtgcccagga | 6575 | | |
| CHST3 | 16 | (intron 1 7967) | gacaggaaccccaccccgag T/G gatgtctggccctgtgacct | 6576 | | |
| CHST3 | 17 | (intron 1 11412) | gcttgcacttctgattcatt C/T tgcagtcactggctctttgt | 6577 | | |
| CHST3 | 18 | (intron 1 11591) | ccctggaagggcctcagtgc G/A gtgactcattacccagcatg | 6578 | | |
| CHST3 | 19 | (intron 1 12541) | acccacacagcatgaatggg G/C ccagccccagcctgcgggct | 6579 | | |
| CHST3 | 20 | (intron 1 12672) | gtagccaaagctggggctgt G/C gggtcagggcatggcaaggg | 6580 | | |
| CHST3 | 21 | (intron 1 14809) | ggatgtgtagggtttgggct C/T ggccttaagggatgggtgga | 6581 | | |
| CHST3 | 22 | (intron 1 16161) | gatgctggtcaggcattgtc G/A gtgggatctttaacaccacc | 6582 | | |
| CHST3 | 23 | (intron 1 16385) | tatttagcatgtgggtttca A/C ctttctgttttttcaaaggg | 6583 | | |
| CHST3 | 24 | (intron 1 33638) | gacttgggccacgtccttgg G/C catgaatcttggtctatgtc | 6584 | | |
| CHST3 | 25 | (intron 1 35145) | agggaagccgaagcctcact T/C gctggggcttgcctggccta | 6585 | | |
| CHST3 | 26 | (intron 1 35340) | tgtgaagttttgcccacagt T/C ggtggccatggttcgcaccg | 6586 | | |
| CHST3 | 27 | (intron 1 35436) | gccactcatgtatggagcaa T/C tgcctttttttcttcctctt | 6587 | | |
| CHST3 | 28 | (intron 1 36150) | acatagaagaggctgggcct G/T aggaagccagggaagcagga | 6588 | | |
| CHST3 | 29 | (intron 1 36194) | ggtgtggggaggccagcagg G/A gcgcgggcctcagcggcgag | 6589 | | |
| CHST3 | 30 | (intron 1 37602) | ctggaacagcaacttaaaaa A/T agaaatagtccctggaaggg | 6590 | | |
| CHST3 | 31 | (intron 1 37725) | gggtagcaagggcagctccc C/T gaccggca c/g ccgccttccca | 6591 | | |
| CHST3 | 32 | (intron 1 37734) | gggcagccccc c/t gacccgga C/G cggccttttcacccctctcc | 6592 | | |
| CHST3 | 33 | (intron 1 38208) | gccattctagatgcgagtcc C/T gactttgggg t/c gctcgcatt | 6593 | | |
| CHST3 | 34 | (intron 2 255) | ctacagctgtgaaaggctag A/G caagatacttaacatttctg | 6594 | | |
| CHST3 | 35 | (3′untranslated region 2 | acacctcagaggagcgtgtg C/A ccaacatttgtaggattatt | 6595 | | |
| CHST3 | 36 | (3′untranslated region 2 | aggcctcatctggggtaggg C/G caagaggaaagtacagagtg | 6596 | | |
| CHST3 | 37 | (3′untranslated region 2 | ctggaactcccccgtagggc C/T cggggaagagtattgcttaa | 6597 | | |
| CHST3 | 38 | (3′untranslated region 2 | cttaacgcaggatgtgctgg G/A tgttttgtttcgggctttta | 6598 | | |
| CHST3 | 39 | (3′untranslated region 2 | gcttggtgtctttcttgttt C/T atggctgtgtttttgctttt | 6599 | | |
| CHST3 | 40 | (3′untranslated region 3 | ccgagggctgcccagctgtg C/T ttctggtttccggggcaatt | 6600 | | |
| CHST3 | 41 | (3′untranslated region 3 | ctgtcagatacggcccatcg T/C aaacccagagggctgcattt | 6601 | | |
| CHST3 | 42 | (3′untranslated region 3 | gttccccatgtggaggtcgg A/G ggggctgggactggggaggg | 6602 | | |
| CHST3 | 43 | (3′untranslated region 3 | ggccctgctaatgtggacag T/C agaccccaggcccccctctt | 6603 | | |
| CHST3 | 44 | (3′untranslated region 4 | ccagatgtgcatagaagcca G/A ccccggccacatacacggca | 6604 | | |
| CHST3 | 45 | (3′untranslated region 4 | caaagcaaatttaggctttt G/A cccggcggcaatacacgcac | 6605 | | |
| CHST3 | 46 | (3′untranslated region 6 | accccaggccggcagggtac G/A agacaccccttcac g/a gcaca | 6606 | | |
| CHST3 | 47 | (3′flanking region 281) | agacaggagtgttgggccag C/T ggccagggggcctggggatg | 6607 | | |
| CHST3 | 48 | (3′flanking region 997) | acctcttaaagtatttgagc C/T ggtgcctgtcatcccaacct | 6608 | | |
| CHST3 | 49 | (intron 1 22595) | cgggagcaggaaaaaaaaaa A/Δ gaataagaagaaaagaggct | 6609 | | |
| CHST3 | 50 | (intron 1 35423-35424) | gctcatgctcacagccactc AT/Δ gtatggagcaa t/c tgcctttt | 6610 | | |
| CHST4 | 1 | (5′flanking region − 1092) | atgaagccttgtgccatctc G/A ctgtgtcgtgccagcacctg | 6611 | | |
| CHST4 | 2 | (5′flanking region − 941) | ctgccagagagaaacaggaa G/A ggaggaagagccacacaatt | 6612 | | |
| CHST4 | 3 | (intron 1 − 150) | caggaaatgatttggagaag G/T actggtgccattgttggcac | 6613 | | |
| CHST5 | 1 | (intron 1 − 144) | ggcctcttaggtttcagcca A/C gacaggtgactcttagcacc | 6614 | | |
| CHST5 | 2 | (intron 2 17) | caacgtcagagcgcttctca T/A tgtccagctcctttgtttct | 6615 | | |
| CHST5 | 3 | (intron 2 139) | aatcccagcactttgggagg C/A ggagatgtgcggatggatca | 6616 | | |
| CHST5 | 4 | (intron 3 1829) | gactgtctgtctgctattca T/C ataggaacaaataattcatg | 6617 | | |
| CHST5 | 5 | (intron 3 2037) | aaatgaaaccaacaccaaca C/G tgcagagaagcaaacaaaag | 6618 | | |
| CHST5 | 6 | (intron 3 2134) | aagcagctaaattgtgttcc G/A tacaggtgcaattaggcagg | 6619 | | |
| CHST5 | 7 | (intron 3 2528) | atggtaaagttcgcctgggt G/A cagtatgtcagcatcctgct | 6620 | | |
| CHST5 | 8 | (intron 3 2674) | gcacttatcctagaaaggcc A/G cccctgaagactcagcagga | 6621 | | |
| CHST5 | 9 | (intron 3 7039) | cgggcccccgccggccaccg T/C gggaccgcagccacgtctga | 6622 | | |
| CHST5 | 10 | (intron 3 7211) | gtagccccaggacaccccca T/G cctcaacatcccattctggg | 6623 | | |
| CHST5 | 11 | (intron 3 7294) | ggagcttccagtggcttggt T/C acccccgactcttcgtccat | 6624 | | |
| CHST5 | 12 | (intron 4 108) | gcagggtcctgcactctgca G/A ggggcaatcacaggtgggag | 6625 | | |
| CHST5 | 13 | (intron 4 402) | agcactggaaaaagtacagt T/C gcacttgtagcggaggtggg | 6626 | | |
| CHST5 | 14 | (intron 4 547) | ctcctgtccccgcattgagg C/G gaaggagcagaggtgagatc | 6627 | | |
| CHST5 | 15 | (intron 4 1142) | gccccaggtctcatagctcc C/G cattggcagtgctgggattt | 6628 | | |
| CHST5 | 16 | (intron 5 1187) | cactgggcagtaattggggc A/G tgggatgggcatgagggccc | 6629 | | |
| HNK-1ST | 1 | (intron 1 139) | gtgttttggcgacttganga C/T ccccctagtccgcaggagta | 6630 | | |
| HNK-1ST | 2 | (intron 1 1020) | acctgagcagaaaattctct T/C cttcgcggaaatgaaaattg | 6631 | | |
| HNK-1ST | 3 | (intron 1 1091) | aagaatttgtaaacatgaca G/A gcaacgggcagctctaggcg | 6632 | | |
| HNK-1ST | 4 | (intron 1 1971) | ctataactatttcaaacata C/T gaaacaggcataattggatt | 6633 | | |
| HNK-1ST | 5 | (intron 1 2096) | acttagaatattcatttacc A/C agaaatccaaatataacctg | 6634 | | |
| HNK-1ST | 6 | (5′untranslated region − 9 | ctacccagtgacaagaggaa C/A caagaacatcagctcagggg | 6635 | | |
| HNK-1ST | 7 | (intron 2 − 530) | agtgggcggaggcgagaagc G/A tcagtgcccaggcctcggct | 6636 | | |
| HNK-1ST | 8 | (intron 2 − 466) | gctacatcttgtcagccagt C/T agaattttaaacacagccag | 6637 | | |
| HNK-1ST | 9 | (intron 2 − 92) | acggaaatatttgtgctgat A/T cctactgactgaaatcacct | 6638 | | |
| HNK-1ST | 10 | (intron 3 152) | cagggcccccgccccggcac G/A ccacagaggcgcgaggggag | 6639 | | |
| HNK-1ST | 11 | (intron 3 312) | cacagtggccccatgccttg C/T agcagggcgcctcccaggct | 6640 | | |
| HNK-1ST | 12 (intron 3 1948) | tcctttgatgtatcaagttt T/C gcgctgaaggttcccagtgt | 6641 | | | |
| HNK-1ST | 13 | (intron 3 2140) | ctacacctggagaggagcac C/T gcagcggtccttaatactgc | 6642 | | |
| HNK-1ST | 14 | (coding region 187 | agaagcacattccggaggaa C/T tgaaggtgggcacagccagg | 6643 | | |
| | | (Leu 63 Leu)) | | | | |
| HNK-1ST | 15 | (intron 4 581) | ccggatcattccctagctgg G/A aggaggggtgcactctggaa | 6644 | | |
| HNK-1ST | 16 | (intron 4 615) | tcgggaaggccgctcacttc G/C taacccccattcgggaccca | 6645 | | |
| HNK-1ST | 17 | (intron 5 7) | gattgttctaaatggtgtgt G/A cgggtctactgaatgtccac | 6646 | | |
| HNK-1ST | 18 | (intron 5 123) | acctgcagggacgggtggcc G/T cccagacaggccggtcggtg | 6647 | | |
| HNK-1ST | 19 | (intron 5 721) | acaaccaggggcccggccca T/C gaaatttagcttcagacagg | 6648 | | |
| HNK-1ST | 20 | (intron 5 867) | cgctgcccacagagtcggtg G/A ccacccctggccactgtttg | 6649 | | |
| HNK-1ST | 21 | (coding region 444 | ccaggagcattttcttccat T/C gaggagatccccgaaaacgt | 6650 | | |
| | | (Ile 148 Ile)) | | | | |
| HNK-1ST | 22 | (intron 6 94) | ctgagttctgtacttggcag A/G ccgatcggaggaccacagag | 6651 | | |
| HNK-1ST | 23 | (intron 6 247) | caggaaggcgacaccacccc G/A ccaacagaaattagcaggca | 6652 | | |
| HNK-1ST | 24 | (coding region 696 | aggaggcaccggacagagac C/G cgggggatccagccggaaga | 6653 | | |
| | | (Thr 232 Thr)) | | | | |
| HNK-1ST | 25 | (coding region 870 | gagaccctggaggacgatgc C/T ccacacaccccaaaagaggc | 6654 | | |
| | | (Ala 298 Ala)) | | | | |
| HNK-1ST | 26 | (3′untranslated region 11 | tcaaacatccctaccagacc T/C ggggctaaccaggtgaagat | 6655 | | |
| HNK-1ST | 27 | (3′untranslated region 11 | ccacacccctcctttgagga C/T gcccggggtctcccacaggc | 6656 | | |
| HNK-1ST | 28 | (3′untranslated region 13 | ggaagcatcacacagcgcta G/A gagccgcctccctcaggcgt | 6657 | | |
| HNK-1ST | 29 | (3′untranslated region 14 | cgaggccccccgggccagcc A/G gggcggcggcacccatcact | 6658 | | |
| HNK-1ST | 30 | (3′untranslated region 15 | gcaagggggctgctgaaatc G/C cagagacttttgcagcatca | 6659 | | |
| HNK-1ST | 31 | (3′untranslated region 16 | gggcggtgcggcgtccaggg G/A cccatctttccagaatccat | 6660 | | |
| HNK-1ST | 32 | (3′untranslated region 18 | aggggaggctttttctacct G/A agaaggggagtgtctttgag | 6661 | | |
| HNK-1ST | 33 | (3′untranslated region 22 | tccagcagtgcggcttcctg G/T c/t aacaaggtaggccctggtg | 6662 | | |
| HNK-1ST | 34 | (3′untranslated region 22 | ccagcagtgcggcttcctg g/t C/T aacaaggtaggccctggtgc | 6663 | | |
| HNK-1ST | 35 | (3′flanking region 1016) | cacacgaaggtgtgcactca C/T ggccggcagggcacccaggt | 6664 | | |
| HNK-1ST | 36 | (3′flanking region 1102) | gcaggcggtgcccaccggga A/C cccccagaagcagggaacag | 6665 | | |
| HNK-1ST | 37 | (3′flanking region 1291) | gccgagaccctcagcaggat A/G gcgcagctacagggcggagc | 6666 | | |
| STE | 1 | (5′flanking region − 605) | caggtttctaaaataataat C/T gaaaggcgagtgatgtttac | 6667 | | |
| STE | 2 | (5′flanking region − 536) | caaaacccccaggccggccg A/G agagttaaaggcaaagagtt | 6668 | | |
| STE | 3 | (5′flanking region ″231) | ccttcttccccaacccctga C/T ggcagacttgggaatttgaa | 6669 | | |
| STE | 4 | (5′untranslated region − 6 | cgcagcttaagatctgcctt G/A gtatttgaagagatataaac | 6670 | | |
| STE | 5 | (intron 1 69) | aaatatagaatgaaaattat G/A caccacaaagctcttaaaaa | 6671 | | |
| STE | 6 | (intron 1 311) | caaggagaaaataaagcaag C/G agggcagaaggaggcagaat | 6672 | | |
| STE | 7 | (intron 1 655) | cctaagaaagtagggactat G/A agaacccctatgtatctata | 6673 | | |
| STE | 8 | (intron 1 672) | ctatgagaacccctatgtat C/T tatatccaccatagtattct | 6674 | | |
| STE | 9 | (intron 1 772) | aaaaggcaggttggaagatg C/A aggaggggagtatgcagaaa | 6675 | | |
| STE | 10 | (intron 1 1715) | taaccatcttgcttaacctt A/G tcatttttagccaagtcatt | 6676 | | |
| STE | 11 | (intron 1 1928) | aaatgatacatattcaggaa A/G tcaaaaatctctgacttaga | 6677 | | |
| STE | 12 | (intron 1 1953) | aaatctctgacttagatacc C/T ggcaataataatcaaatgta | 6678 | | |
| STE | 13 | (intron 1 2087) | aattttgaaagaaattgaag T/G tctgtggtttttatttatca | 6679 | | |
| STE | 14 | (intron 1 2323) | taggtatgtaggagggtccc G/C ttatatacatagttgttaat | 6680 | | |
| STE | 15 | (intron 2 165) | tctattccatgaccacaatt T/G ttacctgtaacttgaatagt | 6681 | | |
| STE | 16 | (intron 2 1707) | cctaggacccaacatgagac A/G taatataccatcagtaaaat | 6682 | | |
| STE | 17 | (intron 3 850) | ggtgtccattccctcaagaa T/G ttatactttgtgttacaccc | 6683 | | |
| STE | 18 | (intron 4 1653) | agtaacaggctagtagataa T/C ataaataactgaggccaacg | 6684 | | |
| STE | 19 | (intron 4 1899) | tacatgaacttagagaatca A/G gtagatcacacacaccaaca | 6685 | | |
| STE | 20 | (intron 4 1930) | cacaccaacaataaaAttac A/G cagaatgataaaagaatttg | 6686 | | |
| STE | 21 | (intron 5 666) | ttctgatcatgtaggaacaa T/C tataaagaaaataataatgt | 6687 | | |
| STE | 22 | (intron 5 982) | aggcaaagcggcAcctcgtg A/C ctcacacaacattatattat | 6688 | | |
| STE | 23 | (intron 7 369) | agattttattcctctctctt T/C ttgagttgaagaaataagtt | 6689 | | |
| STE | 24 | (intron 7 447) | cacctttcaagggtaagtgg C/A aaaaaatagaaattcaaata | 6690 | | |
| STE | 25 | (intron 7 672) | aatcttgctctttgaaccac A/T ctgtcagtgagagtcaggga | 6691 | | |
| STE | 26 | (intron 7 856) | cgttacagaggacttaaaac A/G gttgtcttgcttgcaaacgg | 6692 | | |
| STE | 27 | (3′flanking region 218) | cagcctcccaagtagctagg A/G ctacagacatgtgcaaccat | 6693 | | |
| NQ01 | 1 | (intron 1 80) | aggaggttgtaggggcttgg C/A ctgaattttgttccttgact | 6694 | | |
| NQ02 | 1 | (5′flanking region − 434) | tttctgttgcaccacggacc C/G ccaggctgtaaccgggatac | 6695 | | |
| NQ02 | 2 | (5′flanking region − 406) | gtaaccgggataccagccag A/G gatggggagcgggaggcgca | 6696 | | |
| NQ02 | 3 | (5′intranslated region − 1 | tcctgcggctcctactgggg A/C gtgcgctggtcggaaggtga | 6697 | | |
| NQ02 | 4 | (intron 1 1919) | tcactcaaatagagctgagt T/C agtcactcagctcttagaca | 6698 | | |
| NQ02 | 5 | (intron 1 2004) | acaaactcacatgccaccag C/G catatgatgtaaacatgtaa | 6699 | | |
| NQ02 | 6 | (intron 1 3391) | aaagcagagggctgtgcagg C/T gcccctgcccctagctgagg | 6700 | | |
| NQ02 | 7 | (intron 1 3456) | caaaggcctcatccgcaggg C/A ggccaactctictgttttag | 6701 | | |
| NQ02 | 8 | (intron 1 3595) | actgcccagctttaggttca T/C ccgtgtaagtgttgctggtg | 6702 | | |
| NQ02 | 9 | (intron 1 3596) | ctgcccagctttaggttcat T/C cgggtaagtgttgctggtgt | 6703 | | |
| NQ02 | 10 | (intron 1 3598) | gcccagctccaggcccaccc T/C tgcaagtgctgctggtgtca | 6704 | | |
| NQ02 | 11 | (intron 1 3651) | cccggcctgttgaagggatg A/G atgtgacctctcccacattc | 6705 | | |
| NQ02 | 12 | (intron 1 6036) | tggtgtggcggttcactgat C/T ccccagccttctgctcgatc | 6706 | | |
| NQ02 | 13 | (intron 2 14) | agggcaggtaaggattcact A/G ttgtggagtaagaccctttt | 6707 | | |
| NQ02 | 14 | (intron 2 192) | gccacgtggaagtgtataaa C/T tatcgggaactaccgtgcgg | 6708 | | |
| NQ02 | 15 | (intron 2 635) | cacactgtttagcacctagc A/C ccatcccgggccgcggccca | 6709 | | |
| NQ02 | 16 | (intron 2 685) | agcagcaccccccccccAcc G/A gcggtgacaaaccaaaatgt | 6710 | | |
| NQ02 | 17 | (coding region 139 | ctgatgtgtatgccatgaac T/C ttgagccgagggccacagac | 6711 | | |
| | | (Phe 47 Leu)) | | | | |
| NQ02 | 18 | (intron 3 36) | aatgctctatttataaaaac T/C acccccaggttttccacccc | 6712 | | |
| NQ02 | 19 | (intron 3 728) | aacgtgggcataaaccacca T/C ctagtgccaaaaagcaggtg | 6713 | | |
| NQ02 | 20 | (intron 4 1577) | cgccgctgcacaccccggcc C/T gacaccagccctttctttac | 6714 | | |
| NQ02 | 21 | (intron 4 1832) | ccggccggccacgtggagcc C/T gccttcctccgggcacccac | 6715 | | |
| NQ02 | 22 | (intron 4 2583) | cggtgttacgcacagctcct C/T gtcccctccctgcctgccca | 6716 | | |
| NQ02 | 23 | (coding region 330 | ctgtactggttcagcgtgcc A/G gccatcctgaagggctggat | 6717 | | |
| | | (Pro 110 Pro)) | | | | |
| NQ02 | 24 | (coding region 405 | atcccaggattccacgattc C/T ggcttgccccaggcctgtgc | 6718 | | |
| | | (Ser 135 Ser)) | | | | |
| NQ02 | 25 | (intron 5 21) | gtatgtgctcttggacaagg A/T ccactatggatagttggagg | 6719 | | |
| NQ02 | 26 | (intron 5 253) | atggcaaacaagggagtggg T/C caggtgtcaggtgacggggg | 6720 | | |
| NQ02 | 27 | (intron 6 2435) | ecccccccaaataatctaac T/C gaagggtatgtaacaggtgt | 6721 | | |
| PIG3 | 1 | (5′flanking region − 47) | gggaaggaggaaaggaaaga G/A ggggagggcggttcggctta | 6722 | | |
| PIG3 | 2 | (intron 2 243) | caacaccggacgcccagcag A/C agccccagctccgtagaatc | 6723 | | |
| PIG3 | 3 | (3′flanking region 282) | agcaggccccagccgggccc G/A ccacccaccggggccccacc | 6724 | | |
| PIG3 | 4 | (5′untranslated region − 9 | cccgcgaggatacagcggcc (CCTGY)16 cagacaatatgttagccgtg | 6725 | | |
| PIG3 | 5 | (3′flanking region 625-62 | ctcctcaggccccgcccctt (T) ccattactcacttgggtccc | 6726 | | |
| PIG3 | 5 | (3′flanking region 625-62 | ccccgcaggccccgcccctt ccattactcacttgggtccc | 6727 | | |
| PIG3 | 6 | (3′flanking region 770) | ccacctgggtcccgccccac C/Δ cgccacaaccctgctcaagc | 6728 | | |
| NDUFA1 | 1 | (5′flanking region − 1437) | agggctaaaaatcctgatta T/A acctaccttgaagcttcgaa | 6729 | | |
| NDUFA1 | 2 | (intron 2 3071) | aataaaagtacatggcatat C/A cccgatgggaacagacttgt | 6730 | | |
| NDUFA1 | 3 | (3′flanking region 1218) | aactccatgtgtataaagca A/G caccacagatgacacttcca | 6731 | | |
| NDUFA1 | 4 | (3′flanking region 1411) | ggattgcgccaccccttgac C/T ggcaatgaccttttactttt | 6732 | | |
| NDUFA1 | 5 | (3′flanking region 1411) | ggatcgtgccaccccttgat C/G ggcaatgaccgtttactttt | 6733 | | |
| NDUFA2 | 1 | (intron 2 1087) | aacatacaaaaattagccgg A/G t a/g cggtggcgggcacctgta | 6734 | | |
| NDUFA2 | 2 | (intron 2 1089) | catacaaaaattagccgg a/g t A/G cggcggcgggcacctgtaat | 6735 | | |
| NDUFA2 | 3 | (intron 2 1356) | ttccctgaaacaacccattg T/C ggccatccagaatcagccaa | 6736 | | |
| NDUFA2 | 4 | (3′flanking region 467) | cacagcctcatgggtcagcc C/T actccagagggtgcattccc | 6737 | | |
| NDUFA2 | 5 | (3′flanking region 744) | ggaagcaggggccctggcca C/T agccgctggcagtaagcagg | 6738 | | |
| NDUFA2 | 6 | (3′flanking region 838-83) | catagtctacaaagaatgaa (ACAC) aaagatcataacaatagcta | 6739 | | |
| NDUFA2 | 6 | (3′flanking region 838-83) | catagtctacaaagaaggaa aaagatcataacaata | 6740 | | |
| NDUFA3 | 1 | (intron 2 2656) | tccctgctgccctcccctgc G/A cactttatcttccctttgcc | 6741 | | |
| NDUFA3 | 2 | (coding region 241 (Lec 81 | agggccccagcctggagtgg C/G tgaagaaactgtgagcacct | 6742 | | |
| NDUFA3 | 3 | (3′flanking region 1019) | tccttacctgcactggcacc A/G gctctggagccccagtccct | 6743 | | |
| NDUFA5 | 1 | (intron 3 2155) | agactctagcatggtacctg G/C aacataaggttccttagaaa | 6744 | | |
| NDUFA5 | 2 | (intron 3 2493) | ggcatattgctagttttctc G/T gtctcaatttcatcatctat | 6745 | | |
| NDUFA5 | 3 | (intron 3 2712) | acaaattttgaactgttcac C/T taacacaggctttttctgaa | 6746 | | |
| NDUFA5 | 4 | (3′flanking region 1296) | aggtatctaaaaggtattgc A/C atttggtcattggttctttc | 6747 | | |
| NDUFA5 | 5 | (intron 3 30-31) | aagtcagttttgttgtcttg (GATTTGTGGTATCCAG) | 6748 | | |
| | | | tgtaacatttaaccaaaaaa | | | |
| NDUFA5 | 5 | (intron 3 30-31) | aagtcagttttgttgtcttg | 6749 | | |
| | | | tgtaacatttaaccaaaaaa | | | |
| NDUFA5 | 6 | (intron 3 427-428) | attaagtagcagttaataaa AG/Δ tctagactgctgattcatac | 6750 | | |
| NDUFA5 | 7 | (intron 3 4733-4734) | tataggaattttaaaatata TA/Δ ggatattgaaacattcagtt | 6751 | | |
| NDUFA6 | 1 | (5′flanking region − 1148) | tttataatttatatatgtta C/T gtgctttcttttgtatagct | 6752 | | |
| NDUFA6 | 2 | (5′flanking region − 363) | actacccaggagcgcggcgg G/A cagccggatagcaggacgct | 6753 | | |
| NDUFA6 | 3 | (coding region 26 (Ala 9 V | ggggagcggcgtccgccaag C/T tacttctaccgccagcacct | 6754 | | |
| NDUFA6 | 4 | (intron 1 1318) | attcagcagtttgaaaacat A/G atgtttgcctggcagaatac | 6755 | | |
| NDUFA6 | 5 | (intron 2 562) | agttaaagaatctgaaaagt G/C tcagaaatgatttaccctga | 6756 | | |
| NDUFA6 | 6 | (5′flanking region − 861) | ctgtaaaatggggatgctga (T) ggtacctacctgacctatga | 6757 | | |
| NDUFA6 | 6 | (5′flanking region − 861) | ctgtaaaatggggatgctga ggtacctacctgacctatga | 6758 | | |
| NDUFA6 | 7 | (intron 1 1251-1278) | tgtggggagtgcctgtagca (GT)12-14 ttcggggtggtgcattcaaa | 6759 | | |
| NDUFA7 | 1 | (5′flanking region − 731) | accaaccaaaggtctatcaa A/G ggggtgtcctctttgcaccc | 6760 | | |
| NDUFA7 | 2 | (5′flanking region − 434) | aaagggaaccatcagaeccc C/T gtgatgaaatgagaatcggc | 6761 | | |
| NDUFA7 | 3 | (5′flanking region − 395) | gctcccggattccggctggc A/G ggggttagggcagggtagag | 6762 | | |
| NDUFA7 | 4 | (5′flanking region − 100) | agaggagtcacgtgcttcgg G/A gagagcctttataggacgtt | 6763 | | |
| NDUFA7 | 5 | (intron 1 92) | tcacctccctcctaagccgg G/A acccttcgctctccccgaat | 6764 | | |
| NDUFA7 | 6 | (intron 1 133) | ctccctgggaacccccagct A/G gt c/g accccttcagcccggga | 6765 | | |
| NDUFA7 | 7 | (intron 1 136) | cctgggaacccccagct a/c gt C/G accccttcagcccgggaccc | 6766 | | |
| NDUFA7 | 8 | (intron 2 89) | tcctttagacccctgaaacg G/C agggctgacatcctgccacc | 6767 | | |
| NDUFA7 | 9 | (coding region 196 | gcagccgggaatctgtgccc C/G cttccatcatcatgtagtcg | 6768 | | |
| | | (Pro 66 Ala)) | | | | |
| NDUFA7 | 10 | (intron 3 4203) | gcctccacccctggggcgcc T/G cctccatcaccccaccctcc | 6769 | | |
| NDUFA7 | 11 | (intron 3 4604) | gggccttgtgtacgctggag A/G ccaaaagtgggaagggagga | 6770 | | |
| NDUFA7 | 12 | (5′flanking region | agggtccagggtcccctgct CAGAGGCT/Δ aacactggccgaagagaa | 6771 | | |
| | | (−1353)-(−1360)) | | | | |
| NDUFA7 | 13 | (5′flanking region | agccctgatccacccactct CT/Δ gaaacttctctgctaataaa | 6772 | | |
| | | (−1233)-(−1234)) | | | | |
| NDUFA7 | 14 | (intron 2 4142-4143) | cattttgtgactgaggtgac AG/Δ gggcccacagcggggccatg | 6773 | | |
| NDUFA8 | 1 | (intron 1 − 75) | tttgtgttctctattctgac C/T cgcatgaggtaaagctgaga | 6774 | | |
| NDUFA8 | 2 | (intron 2 790) | caaacctagacaaagtgtgc C/T ctttatccagaagtgagcag | 6775 | | |
| NDUFA8 | 3 | (intron 2 900) | ttcaggagataaaaagctct G/A attgctcaggcctgagatgg | 6776 | | |
| NDUFA8 | 4 | (intron 2 3837) | gaagttgtcttgtaagtgag A/G taagaatatgtactcacata | 6777 | | |
| NDUFA8 | 5 | (intron 2 3942) | tcattgttttgcaaagagat G/T cccctaacccagctttcttt | 6778 | | |
| NDUFA8 | 6 | (intron 3 − 66) | gaggagacaccaggaggcgc A/G ttgatggttacagattcctc | 6779 | | |
| NDUFA8 | 7 | (3′untranslated region 52 | tttatttctggaccaagtaa A/G gatgggtccgtggcccacac | 6780 | | |
| NDUFA8 | 8 | (3′flanking region 367) | gtcatacaaggggagcctcc A/G ggatagaagtgcagaeactt | 6781 | | |
| NDUFA8 | 9 | (3′flanking region 777) | attcttttttcactactagg C/T tgtttcctccacatctgact | 6782 | | |
| NDUFA8 | 10 | (3′flanking region 1053) | aaagaaaaagcaetgtgtga T/A ctgccatggccgcttctgca | 6783 | | |
| NDUFA8 | 11 | (3′flanking region 1190) | gattctctaatgaaaaataa G/T acttttttttgcattttttt | 6784 | | |
| NDUFA8 | 12 | (intron 2 449-453) | tcattgtgcatgatacttaa GTAAA/Δ aaaaaactaagctgtgtaat | 6785 | | |
| NDUFA8 | 13 | (intron 2 455-459) | tgcatgatacttaagtaaaa AAAAA/Δ ctaagctgtgtaattgtagg | 6786 | | |
| NDUFA8 | 14 | (intron 2 707-708) | tcattttggaaagactctca (A) ccttgctgtaccaaaaatgg | 6787 | | |
| NDUFA8 | 14 | (intron 2 707-708) | tcattttggaaagactctca ccttgctgtaccaaaaatgg 6788 | | | |
| NDUFA9 | 1 | (5′flanking region − 807) | gatggctctttgtagaacaa T/C gcagattctcaaaggtgacc | 6789 | | |
| NDUFA9 | 2 | (5′flanking region − 769) | accacagttaaagaaaaaat T/C anaagccattgcgctagaga | 6790 | | |
| NDUFA9 | 3 | (5′flanking region − 353) | cacaccctattttggtttct C/G ttctccacttttcccctcgt | 6791 | | |
| NDUFA9 | 4 | (5′flanking region − 322) | ttcccctcgttcttgtcccc C/T ctcctccccctcctgggccc | 6792 | | |
| NDUFA9 | 5 | (intron 1 447) | attcatatgagcacaatgga A/G atgataatattacaatacca | 6793 | | |
| NDUFA9 | 6 | (intron 1 1039) | ggcttgatgttcagcctgag G/A caagaattaggagtgtttag | 6794 | | |
| NDUFA9 | 7 | (intron 1 4010) | aatgtatccaaaagagactc T/G cattcctgccatatgaagaa | 6795 | | |
| NDUFA9 | 8 | (intron 3 49) | gacaaatataaattactaag G/A tcatttctaggagtgatagg | 6796 | | |
| NDUFA9 | 9 | (intron3 107) | aatttcttcccagaatggac C/T aaaggcaccctccgttccca | 6797 | | |
| NDUFA9 | 10 | (intron 3 1183) | atctctggtaatattcatac A/G gattatttgtaatcccttta | 6798 | | |
| NDUFA9 | 11 | (intron 3 1395) | attcctagttctttgtccct C/T aagtttgttggtcaccttgt | 6799 | | |
| NDUFA9 | 12 | (intron 3 2363) | agaaaatagtcatgaatggc C/T ccaactaacaccagtcttta | 6800 | | |
| NDUFA9 | 13 | (intron 3 2608) | gtcatttgattacctgagta A/C agtgtactgttacctgtttg | 6801 | | |
| NDUFA9 | 14 | (intron 4 561) | attttataaattctttgatg A/C cttgggggtcttattcaact | 6802 | | |
| NDUFA9 | 15 | (intron 4 860) | attgtgtagagtaatgacag C/T agagctgtcaacttttttaa | 6803 | | |
| NDUFA9 | 16 | (intron 4 879) | gcagagctgtcaactttttt A/T aaaaaataattttagcttaa | 6804 | | |
| NDUFA9 | 17 | (intron 4 893) | ttttttaaaaaaataatttt A/G gcttaaaaaaattaaaaatt | 6805 | | |
| NDUFA9 | 18 | (intron 4 1090) | atcattgctgtttaaaagtt T/C aagtagtgtgaatttcagta | 6806 | | |
| NDUFA9 | 19 | (intron 4 1188) | aaccaatccttttatttttt A/T tcttccagaaactttgattt | 6807 | | |
| NDUFA9 | 20 | (intron 5 161) | gggtgtgtgtgatgttttga C/T gttttgattgattgccttct | 6808 | | |
| NDUFA9 | 21 | (intron 5 373) | ctttctcaccgcttgcactg C/T agtggttttgtgccactctt | 6809 | | |
| NDUFA9 | 22 | (intron 5 457) | gccagggaagatgcctattc A/C cacagtgcttatgctccttt | 6810 | | |
| NDUFA9 | 23 | (intron 5 3113) | gatttttctccttcttcaat G/A taagcttcccttaaaataaa | 6811 | | |
| NDUFA9 | 24 | (intron 5 3339) | tctaaactcaaaacaggttt G/A tttggttattgtttaggctg | 6812 | | |
| NDUFA9 | 25 | (intron 6 414) | tatagttttgccttttccag G/C atattacatatatggttaga | 6813 | | |
| NDUFA9 | 26 | (intron 6 518) | ctttcatttcttttcatagc T/C tgatagctcatttctttata | 6814 | | |
| NDUFA9 | 27 | (intron 7 974) | ggattatgcgtacttggaaa A/G tacttggatagcggtgatta | 6815 | | |
| NDUFA9 | 28 | (intron 8 368) | acattaattttgatggagta T/G cacaatgcctccagaggctg | 6816 | | |
| NDUFA9 | 29 | (intron 8 954) | gcatgccatgagttatatag T/C ctagataagaattacaattc | 6817 | | |
| NDUFA9 | 30 | (intron 8 1253) | tcctcttgaaattgtagata G/T gtatctacacatttctcatc | 6818 | | |
| NDUFA9 | 31 | (intron 8 11608) | gaaaagatagatgtataaat G/A accaaaaattcgtgaagaaa | 6819 | | |
| NDUFA9 | 32 | (intron 8 11930) | ctacaaatatattctaaatg C/T gtaatcatggataagtacaa | 6820 | | |
| NDUFA9 | 33 | (intron 9 1998) | tgtttttcaagcctttaaac G/A gctgtggaaccctgtgctca | 6821 | | |
| NDUFA9 | 34 | (intron 9 2239) | ccagctacttgggaggctga A/G gtgggaggatcacttgagcc | 6822 | | |
| NDUFA9 | 35 | (intron 9 2885) | acagcggtctgtcttcctgc A/G gttctcataggctagcttac | 6823 | | |
| NDUFA9 | 36 | (intron 10 801) | tacactaaagtgtctcttac G/A tttatacttgagaaagtgtt | 6824 | | |
| NDUFA9 | 37 | (intron 10 910) | tgcagactttcaggtgggta G/C gatgagggattgctgctgct | 6825 | | |
| NDUFA9 | 38 | (intron 10 1180) | aaaactgagtcagaacgccg G/A tgctcagaaaacaggggcgt | 6826 | | |
| NDUFA9 | 39 | (3′flanking region 554) | gtgccagcacttaggaatta T/G gaccttctaatgaagttctt | 6827 | | |
| NDUFA9 | 40 | (5′flanking region | taaacagtaggggcaagata (TC) gagtggaaacagccaagatt | 6828 | | |
| | | (−1129)-(−1128)) | | | | |
| NDUFA9 | 40 | (5′flanking region | taaacagtaggggcaagata gagtggaaacagccaagatt | 6829 | | |
| | | −1129)-(−1128)) | | | | |
| NDUFA9 | 41 | (5′flanking region − 341) | tggtttct c/g ttctccacttt T/Δ cccctcattcttatcccc c/ | 6830 | | |
| NDUFA9 | 42 | (intron 4 594) | attcaactttttatcccccc T/Δ aatgattaacatagtgtatt | 6831 | | |
| NDUFA9 | 43 | (intron 10 356-375) | taacttcctcctaacgtcct GAAGAAACTGTTGACAGTTT/Δ | 6832 | | |
| | | | cttccttctttctttaacct | | | |
| NDUFA9 | 44 | (intron 10 379-381) | gaaactgttgacagtttctt CCT/Δ tctttctttaacctactcca | 6833 | | |
| NDUFA9 | 45 | (intron 10 384-387) | tgttgacagtttcttccttc TTTC/Δ tttaacctactccagtcagg | 6834 | | |
| NDUFA9 | 46 | (intron 10 436-437) | ccatttctcccctaaacttg (TTCTTTTAAAATTG) ctcttttcaaaat | 6835 | | |
| NDUFA9 | 46 | (intron 10 436-437) | ccatttctcccctaaaattg ctcttttcaaggt | 6836 | | |
| NDUFA9 | 47 | (intron 10 495-496) | gccacatccaatggtcagtt (TTCAGGCCTTT) ctcagacctcatgtca | 6837 | | |
| NDUFA9 | 47 | (intron 10 495-496) | accacatccaatagtcagtt ctcaaacctcatgtcc 6838 | | | |
| NDUFA9 | 48 | (intron 10 519-520) | gacctcatgtcatgtgcctg (GGCCTG) tgcatttgcttctagggagg | 6839 | | |
| NDUFA9 | 48 | (intron 10 519-520) | gacctcatgtcatgtgcctg tgcatttctgtctagggagg | 6840 | | |
| NDUFA9 | 49 | (intron 10 558-559) | gatgcaaaataaaataaaaa (A) tactataccaataccacatc | 6841 | | |
| NDUFA9 | 49 | (intron 10 558-559) | gatgcaaaataaaataaaaa tactataccaataccacatc | 6842 | | |
| NDUFA10 | 1 | (5′flanking region − 1734 | tgcaccttgaactgtttact T/C tcctgtaaccatttaccctt | 6843 | | |
| NDUFA10 | 2 | (5′flanking region − 1492 | aaaacatccacgcaaacagg T/C tgtgagaagttacgtctgcg | 6844 | | |
| NDUFA10 | 3 | (intron 3 370) | aagactgtgcatgtgccatg C/A agacagagatgtggatgcca | 6845 | | |
| NDUFA10 | 4 | (intron 3 2485) | ttgttattttcttttctctg G/A aatgcagtgatcagttgaca | 6846 | | |
| NDUFA10 | 5 | (intron 4 236) | ctgtgaaagcagattggagc C/T ctggacctcaaacacacgca | 6847 | | |
| NDUFA10 | 6 | (intron 4 1742) | tgtcggcatctgctgagtgt C/T tgctgaagtctgaggactgg | 6848 | | |
| NDUFA10 | 7 | (intron 4 2090) | ggctgggggaaagcagatca T/C gttggctaaaggacaggtgg | 6849 | | |
| NDUFA10 | 8 | (intron 4 3054) | cagctgattatactactgaa A/C cgggataaatg c/t agcttgat | 6850 | | |
| NDUFA10 | 9 | (intron 4 3066) | ctactgaa a/c cgggataaatg C/T agcttgatgattttcagctg | 6851 | | |
| NDUFA10 | 10 | (intron 4 3377) | gtcacagtttaaatgctgct G/A ttttactctgtgtaagtagc | 6852 | | |
| NDUFA10 | 11 | (intron 5 46) | aagcatctctattttgaatg T/C agatcagcactaaaagccct | 6853 | | |
| NDUFA10 | 12 | (intron 8 1465) | gcaacacccagttcctggta C/T aggcctcatatccagcgtgc | 6854 | | |
| NDUFA10 | 13 | (intron 8 1809) | cctggaggcacaaggatggc C/A ggggcactcaacttccctct | 6855 | | |
| NDUFA10 | 14 | (intron 8 11226) | gttgtgtgactgtgtggggc A/G tctcacctctcgggctgcag | 6856 | | |
| NDUFA10 | 15 | (intron 8 11319) | atcttgccttccctcctgcc G/A tctgttcaggcttgaatcct | 6857 | | |
| NDUFA10 | 16 | (intron 8 11386) | ccataatcctagcttgaacc C/T tcctttttccctgctgaccc | 6858 | | |
| NDUFA10 | 17 | (intron 8 13361) | ccaggccactgattgctttc G/A cattttctagcattttctta | 6859 | | |
| NDUFA10 | 18 | (intron 9 183) | tttctgtgtggaaagctgat G/A aagtcctcagatgacagccc | 6860 | | |
| NDUFA10 | 19 | (intron 9 8028) | gaggacattccacagaacgt G/A tgactattagagcagaaggt | 6861 | | |
| NDUFA10 | 20 | (intron 9 10742) | ctggaggagaggggtggagc C/G agttcagccagcactggggt | 6862 | | |
| NDUFA10 | 21 | (intron 9 13908) | cacattgttatgtaaccaag C/T ct g/t gaattgcagtgtgaaga | 6863 | | |
| NDUFA10 | 22 | (intron 9 13911) | attgttatgtaaccaag c/t ct G/T gaattgcagtgtgaagaact | 6864 | | |
| NDUFA10 | 23 | (intron 9 14064) | tcttgactattagaaaccct A/G tcagataaattttaaaacag | 6865 | | |
| NDUFA10 | 24 | (intron 9 14184) | tggctttggttgggaacagc G/A agagatacagaaccgacggt | 6866 | | |
| NDUFA10 | 25 | (intron 9 16487) | cttgaagctgatcgttccct C/A cttgaagctgatcgttccct | 6867 | | |
| NDUFA10 | 26 | (intron 9 16779) | gccagacgtgactgctttag G/A ttcctcatgacattcagacc | 6868 | | |
| NDUFA10 | 27 | (intron 9 17663) | ttccaaatcaccccagaact T/G tgcagtattttgaagctcct | 6869 | | |
| NDUFA10 | 28 | (5′flanking region | gtaaaattgttttaactaga (C)9-11 ttcctaaaccaaggtataaa | 6870 | | |
| | | (−1668)-(−1609) | | | | |
| NDUFA10 | 29 | (5′flanking region | ctgtatccattggaaggcac (A)15-21 tgcaaaggaaacaaggcaaa | 6871 | | |
| | | (−1355)-(−1334) | | | | |
| NDUFA10 | 30 | (intron 1 46-61) | tggcggggtggcagggtggc GGGGTGGCGGGGTGGG/Δ gagcagttcc | 6872 | | |
| NDUFA10 | 31 | (intron 4 2486) | ctcactggaacttttttttt T/Δ aatttaatttttaaaatttt | 6873 | | |
| NDUFA10 | 32 | (intron 7 1600-1601) | cacttccattctgactgtta (A) cggtgtgattcttcctgcca | 6874 | | |
| NDUFA10 | 32 | (intron 7 1600-1601) | cacttccattctgactgtta cggtgtgattcttcctgcca | 6875 | | |
| NDUFA10 | 33 | (intron 9 1054) | gcgcgtgctgtttctccctt A/Δ tctgtccttgtacacgtgtg 6876 | | | |
| NDUFA10 | 34 | (intron 9 8161-8172) | tttctcgctttctgggagac (T)10-12 aatgttgaaaatatgtgttt 6877 | | | |
| NDUFA10 | 35 | (intron 9 8646-8647) | aattcccccattgcttctct (TT) ctgtagacattttaaaccta | 6878 | | |
| NDUFA10 | 35 | (intron 9 8646-8647) | aattcccccattgcttctct ctgtagacattttaaaccta | 6879 | | |
| NDUFA10 | 36 | (intron 9 16503-16523) | ccct c/a cttgaagctgatcgt TCCCTCCTTGAAGCTGATCGT/Δ | 6880 | | |
| | | | gtccaagatagttgctagga | | | |
| NDUFA10 | 37 | (intron 9 17905-17936) | caaatatatgtatacatgta (CA)12-18 tccttcatgaaaactctttc | 6881 | | |
| NDUFAB1 | 1 | (intron 1 8451) | cagcaccctgtagaggcctc G/A ggatgctgaagatgccatga | 6882 | | |
| NDUFAB1 | 2 | (intron 1 8495) | gacacaggcattctgcagac G/A ccagacaattttagtggcag | 6883 | | |
| NDUFB3 | 1 | (5′flanking region − 1439) | ttaaaagttgacttttttct G/A cc g/a ggcacggtggctcacgc | 6884 | | |
| NDUFB3 | 2 | (5′flanking region − 1436) | aaagttgacttttttct g/a cc G/A ggcacggtggctcacgcctg | 6885 | | |
| NDUFB5 | 1 | (5′flanking region − 213) | ggcggatgaaactctcctac A/C aagaagggccaaaccggccg | 6886 | | |
| NDUFB5 | 2 | (intron 1 6288) | ggggatgttgattacctagg T/C cagtaaagtaaagaaggcat | 6887 | | |
| NDUFB5 | 3 | (intron 1 − 1581) | ctcctgggccacggtatcct A/G tcccttcccctcgttaccct | 6888 | | |
| NDUFB5 | 4 | (intron 1 − 1487) | ccctcttagaccgtatatag T/G cctagcataggatctgcaca | 6889 | | |
| NDUFB5 | 5 | (intron 2 556) | gtgtctggaccatctgccac G/A gtagataaagctctgcatca | 6890 | | |
| NDUFB5 | 6 | (intron 3 467) | ggcgccatcgcactccagcc C/T gggcaacagagtgagactct | 6891 | | |
| NDUFB5 | 7 | (intron 3 497) | agcgaggctctgcccccccc C/G caaaaaaaaactataatcct | 6892 | | |
| NDUFB5 | 8 | (coding region 397 | acaacagccctgaaaagata T/C aggaaagaacaatggccgtc | 6893 | | |
| | | (Tyr 133 His)) | | | | |
| NDUFB5 | 9 | (intron 1 213-215) | attagcatttctaaaacgtt GTT/Δ attcaccatcccaattaatg | 6894 | | |
| NDUFB7 | 1 | (intron 1 68) | cctgaacacctggcacccca G/A ggctggcaccccagggctgg | 6895 | | |
| NDUFB7 | 2 | (intron 2 266) | gggctctctaggggcctgtt T/C gatggggacagggcaggtgg | 6896 | | |
| NDUFB7 | 3 | (intron 1 4480-4481) | agttctgaggctgagagaga (GA) ggccacgccgccggccagtg | 6897 | | |
| NDUFB7 | 3 | (intron 1 4480-4481) | agttctgaggctgagagaga ggccacgccgccggccagtg | 6898 | | |
| NDUFS1 | 1 | (5′flanking region − 3) | tcctagggggtcgtcgtggt C/G cagacagtttagcagaacag | 6899 | | |
| NDUFS1 | 2 | (intron 1 445) | gtgttagcaatggctcacgc T/C cctgtttgttgtccttgttt | 6900 | | |
| NDUFS1 | 3 | (intron 1 470) | cttgttgtccttgtttgttt G/T gcccaccgaccacgctggac | 6901 | | |
| NDUFS1 | 4 | (intron 1 502) | acgttggacagcattttttt A/G ttcctttaactaacgggaaa | 6902 | | |
| NDUFS1 | 5 | (intron 1 557) | ttttgaaaagttagcccagg A/G ttgcattgcaaataacaaaa | 6903 | | |
| NDUFS1 | 6 | (intron 1 5218) | tatctcagaatatctcagga A/G catttagtagacagctatgc | 6904 | | |
| NDUFS1 | 7 | (intron 3 1371) | aagccctaaaatagatagtg T/G caatgggaatgaaaacaaga | 6805 | | |
| NDUFS1 | 8 | (intron 5 414) | ttttgaaacgaggtctcacc A/G tgttgtccaggctgggcttg | 6906 | | |
| NDUFS1 | 9 | (intron 10 812) | gagtgcggtggcgcgatctc G/A acctcgggtcactgcagcct | 6907 | | |
| NDUFS1 | 10 | (intron 11 233) | ggaggccaaggcaggcagat C/T gcccaagcgcaggagtttga | 6908 | | |
| NDUFS1 | 11 | (intron 11 263) | ggccaacatggcgaaacccc G/A cctctactaaaaatacaaaa | 6909 | | |
| NDUFS1 | 12 | (intron 11 585) | ctgtatgtcttaattttaaa G/T taaatttgcatttcatatat | 6910 | | |
| NDUFS1 | 13 | (coding region 1251 | gcaccactgtttaatgctag A/G actcgaaagaggttggcaac | 6911 | | |
| | | (Arg 417 Arg)) | | | | |
| NDUFS1 | 14 | (intron 13 5159) | atgacctttagaaaacgcgc T/C ccagcggatacccaggcata | 6912 | | |
| NDUFS1 | 15 | (intron 14 250) | aaaaagtgttatattagtta C/T accttggtccaaaaattgca | 6913 | | |
| NDUFS1 | 16 | (intron 14 550) | gataaagtctcactatgttg C/T ccaggccgatcccaaactcc | 6914 | | |
| NDUFS1 | 17 | (intron 14 2429) | ctgaaaatacaaaaactagc C/T gggcgcggtggcatgtgcct | 6915 | | |
| NDUFS1 | 18 | (intron 14 2530) | ttacagtgagccgagatcac G/T ccactgcgctccagcctggg | 6916 | | |
| NDUFS1 | 19 | (intron 14 2659) | acacacttaattttttacat T/C gaaaacactgcagctagggt | 6917 | | |
| NDUFS1 | 20 | (intron 16 150) | agaaaacatgcaggcagaaa C/T aggaaggcaaggttacagtg | 6918 | | |
| NDUFS1 | 21 | (intron 18 279) | cactgtgtagcaatttatgg T/C gaatcttccaaagtggcaaa | 6919 | | |
| NDUFS1 | 22 | (3′flanking region 182) | cccaggacaactacaactaa T/A aacaaccacagtaacaaggg | 6920 | | |
| NDUFS1 | 23 | (intron 12 3226) | aaatgtattgtctgtgcttt T/Δ aacatttttgtaatagtaaat | 6921 | | |
| NDUFS3 | 1 | (5′flanking region − 194) | tctgccacaaggagctagga C/T cacgctcaccgcacgattcc | 6922 | | |
| NDUFS3 | 2 | (intron 1 46) | cggggtcaggcgcagcggcg T/C gcccagcgcagagagccccg | 6923 | | |
| NDUFS3 | 3 | (intron 6 − 439) | aaagctgtgtcaaaggtacc G/A ctttagatctggactgtgaa | 6924 | | |
| NDUFS3 | 4 | (intron 6 − 280) | ggcgggcgagcagccagccc G/A gagccccggatgcgggagcg | 6925 | | |
| NDUFS4 | 1 | (5′flanking region − 439) | aactgaacacagccctgccc T/A gagggcttgcaaagtgaatc | 6926 | | |
| NDUFS4 | 2 | (intron 1 1829) | gaaaaaaaaccctaaggcca G/T ggaagacgtcccctaaatac | 6927 | | |
| NDUFS4 | 3 | (intron 1 2057) | attaatgggaaaatctacat C/G caaaactcactttattgtaa | 6928 | | |
| NDUFS4 | 4 | (intron 1 − 521) | ctcatttcaaccaacttcat T/G tcccccactttgcgaagggg | 6929 | | |
| NDUFS4 | 5 | (intron 3 − 1259) | ataaaattatgatattatta G/A cactaacacagccagccaca | 6930 | | |
| NDUFS4 | 6 | (intron 3 − 1174) | aatatatataattataggaa T/C ctcagagtagcaaccatggt | 6931 | | |
| NDUFS4 | 7 | (intron 4 10682) | cacaatataggcacaaactt A/C ctaccaaagccctaacaagc | 6932 | | |
| NDUFS4 | 8 | (intron 4 12299) | cttactatatagatataggg A/T atagactatagagtatctct | 6933 | | |
| NDUFS4 | 9 | (intron 4 12560) | accaaataaggtattatgca G/A gctcacctccccacataaga | 6934 | | |
| NDUFS4 | 10 | (intron 4 18801) | ggaaagacttgccgggccag T/C gcacccgaaaccgcggccat | 6935 | | |
| NDUFS4 | 11 | (intron 4 19888) | ccgcacagctgagaagagca A/G ggggcgggtttccagtaccc | 6936 | | |
| NDUFS4 | 12 | (intron 4 20178) | agaaaagatgagtataattc G/A tctaacttacccattcttaa | 6937 | | |
| NDUFS4 | 13 | (intron 4 23016) | ctactctgtgaaagtaaggt T/A atgttgaacaagtaaattaa | 6938 | | |
| NDUFS4 | 14 | (intron 4 23124) | sctttctttcgagatggagt T/A ccagcagttgggaatgtaat | 6939 | | |
| NDUFS4 | 15 | (intron 1 766) | tgtgatgatttttttttttt T/Δ ggctgtattaaccttccatt | 6940 | | |
| NDUFS4 | 16 | (intron 1 1261) | tttctttctctttttttttt T/Δ gagatacattctcactctga | 6941 | | |
| NDUFS4 | 17 | (intron 4 1974+ 19745) | ctcatcatttaggtgctggt (T) agttgggtttgtcgcaaatc | 6942 | | |
| NDUFS4 | 17 | (intron 4 1974+ 19745) | ctcatcatttaggtgctggt agttgggtttgtggcaaatc | 6943 | | |
| NDUFS5 | 1 | (intron 1 388) | ccaaacatacccagcacttc C/T ggctgtaactccggcctgtt | 6944 | | |
| NDUFS5 | 2 | (intron 1 − 13082) | agtgagccgagattgcacca G/A tgcattccaccctgggcaac | 6945 | | |
| NDUFS5 | 3 | (intron 1 − 12905) | gttttcaacaaaggactcca G/T agtagtagagaactttctgt | 6946 | | |
| NDUFS5 | 4 | (intron 1 − 12564) | attttcatcacacctcaact T/G aaggtataacagccttaaga | 6947 | | |
| NDUFS5 | 5 | (intron 1 − 12561) | ttcatcacacctcaacttaa G/A gtataacagccttaacaatc | 6948 | | |
| NDUFS5 | 6 | (intron 1 − 10561) | aacaatgtggtatagtgggg C/G gggtggtgagcaggtgtcat | 6949 | | |
| NDUFS5 | 7 | (intron 1 − 9065) | cctgatgctcctggctccag G/A gtagaccttttccctttaga | 6950 | | |
| NDUFS5 | 8 | (intron 1 − 8871) | tcaccacgtctctgtagata T/C aggaccgcagaccttcgctt | 6951 | | |
| NDUFS5 | 9 | (intron 1 − 7312) | aaatccttggcttctagaat G/T ggtcactgatggtatataat | 6952 | | |
| NDUFS5 | 10 | (intron 1 − 6827) | aacctctgcctccccgattc A/G ccccattctcctgcctcacc | 6953 | | |
| NDUFS5 | 11 | (intron 1 − 6725) | agtagagacggggtttcacc G/A tgttagccagcatggtctcg | 6954 | | |
| NDUFS5 | 12 | (intron 1 − 6631) | aggcgtcagccactccgccc G/A gcctagaccttcttcttata | 6955 | | |
| NDUFS5 | 13 | (intron 1 − 6531) | cccaacagctcccaatgtaa A/G acagatctcttaatattctg | 6956 | | |
| NDUFS5 | 14 | (intron 1 − 6346) | gcaacagatcttgacctata T/C cccatagggtacacctgagg | 6957 | | |
| NDUFS5 | 15 | (intron 1 − 6327) | atcccatagggtacagctga G/C gactttaatcagaaaaggag | 6958 | | |
| NDUFS5 | 16 | (intron 1 − 6122) | tagccttgcttttactctac T/C gttcctcccaaatcacaccc | 6959 | | |
| NDUFS5 | 17 | (intron 1 − 2512) | acaaactcttaatgcgaatt T/C tgcagatcaaagtgggctta | 6960 | | |
| NDUFS5 | 18 | (intron 1 − 1945) | tttaatctcctttaaatttc G/A caatttcacaacctagggta | 6961 | | |
| NDUFS5 | 19 | (intron 2 75) | tttttttttttttttgagac G/A aagtctcactcttgtcccct | 6962 | | |
| NDUFS5 | 20 | (intron2 148) | ctgtagcctctgcctcccag G/A ttcaggcgattcgcgtacct | 6963 | | |
| NDUFS5 | 21 | (3′flanking region 150) | cagattcaagtggttctcct G/C cctcagcctcccaagtagct | 6964 | | |
| NDUFS5 | 22 | (intron 1 (− 10682)-(− 10681) | attataaacactaaacaaac AT/Δ gtgtggtctctttacagggg | 6965 | | |
| NDUFS5 | 23 | (intron 1 − 10267) | caagtgactaccctgaaaaa A/Δ gaagagatgaaacaaatcac | 6966 | | |
| NDUFS5 | 24 | (intron 1 − 2069) | accagacagagttcccttta C/Δ ttgttttcctgtggcaaaga | 6967 | | |
| NDUFS6 | 1 | (intron 1 26) | ggccgctgggtacaggatgc A/C ccttcctccagccgcccctc | 6968 | | |
| NDUFS6 | 2 | (intron 2 1076) | ggatcatggtggtggagagg G/A ctgtgtgtctggtgggtttg | 6969 | | |
| NDUFS6 | 3 | (intron 2 1260) | cagttgtcgagtaagtggtg T/C atagggtaagtgctctttct | 6970 | | |
| NDUFS6 | 4 | (intron 2 1413) | caaaggagctcatggcattg C/T gaatgggacatttcttccgt | 6971 | | |
| NDUFS6 | 5 | (intron 2 1568) | tggagaaggggaggtttctc T/C tagtgtggatgcggtatgct | 6972 | | |
| NDUFS6 | 6 | (intron 2 1692) | gaccgtggtgacggaggttt C/T ctgggcatcgatgggtggtt | 6973 | | |
| NDUFS6 | 7 | (intron 2 6488) | tagcttaaataattattggc A/G ttcatgttcagaatgcctga | 6974 | | |
| NDUFS6 | 8 | (intron 2 6563) | tttaaacttttattttaaat G/A tccatgaatggggtcggtat | 6975 | | |
| NDUFS6 | 9 | (intron 2 6740) | aaagatttaaacctacatat C/T tttatgcccaatcatttgat | 6976 | | |
| NDUFS6 | 10 | (intron 2 6832) | gcgagggactcattttacag A/T ggttggacacttcactgtgt | 6977 | | |
| NDUFS6 | 11 | (intron 2 7054) | ttcactgccggagcttggcc G/A tgtgaacccggagccgggct | 6978 | | |
| NDUFS6 | 12 | (intron 2 7186) | ggtcagggtcacccttgagc T/C gcgcacactaaatgacggga | 6979 | | |
| NDUFS6 | 13 | (intron 2 7225) | gagggcatcccgcgtcagtc G/A ccagtgtcgaggcgtcagca | 6980 | | |
| NDUFS6 | 14 | (intron 2 7810) | cttccactctggggcgggga C/T gctgtagaaggagcacaaag | 6981 | | |
| NDUFS6 | 15 | (intron 2 11080) | gtaactgttcagtgctttct C/T ctttggatttcatgtaaatc | 6982 | | |
| NDUFS6 | 16 | (intron 2 11657) | gggacagaacgatgtggtgg G/A gagaagagggcgtggcagag | 6983 | | |
| NDUFS6 | 17 | (intron 3 208) | cgaaaaccccctttcaactg T/C gaagtggtgggcggcatgtt | 6984 | | |
| NDUFS6 | 18 | (intron 3 1031) | ctagagtgggactgggcacc C/T ggcatgtcccctcctgggct | 6985 | | |
| NDUFS6 | 19 | (3′flanking region 270) | gcttcagagagccaaggtgg G/C tcttgaggtgcatagtgaag | 6986 | | |
| NDUFS8 | 1 | (5′untranslated region − 4 | agtgtagcctccgcctcccg A/C ttgactggcctgcttggcaa | 6987 | | |
| NDUFS8 | 2 | (intron 1 163) | aggtgcagcggggagccggc T/C ctcagggcgcatgcgccgcc | 6988 | | |
| NDUFS8 | 3 | (intron 3 123) | tctctgagcctgtttccact T/C ttaaaatgattatggtgatg | 6989 | | |
| NDUFS8 | 4 | (intron 5 − 505) | aggcaaggcaggccgggcac G/A gtggctcacgcttgtaatcc | 6990 | | |
| NDUFS8 | 5 | (3′flanking region 491) | ggccctgagctggcctgcgt C/A cagccacatcctctttcctg | 6991 | | |
| NDUFS8 | 6 | (3′flanking region 693) | ttcacttcatttgcagtgag G/A aaaccagctccgagaggtga | 6992 | | |
| NDUFS8 | 7 | (3′flanking region 1267) | ttttcccagacgtaaccgcc G/A tcagagcgtggcatggagcc | 6993 | | |
| NDUFS8 | 8 | (3′flanking region 1362) | cgctgggttctttcccttac C/T gtggtctcccaggcacttac | 6994 | | |
| NDUFS8 | 9 | (3′flanking region 1449) | tgtcagaacaggcctatggc G/A cccaaccacaagtcccccaa | 6995 | | |
| NDUFS8 | 10 | (3′flanking region 1572) | cagccccacaggcctgtgct C/A gctgtgtggggcttagggat | 6996 | | |
| NDUFS8 | 11 | (3′flanking region 783-78 | cagagaccttgacccccccc (C) atctaccatcatttccaaaa | 6997 | | |
| NDUFS8 | 11 | (3′flanking region 783-78 | cagagaccttgacccccccc atctaccatcatttccaaaa | 6998 | | |
| NDUFV1 | 1 | (intron 3 670) | ctgggtggagtggggtggca T/C ggagttgaagacccagtcct | 6999 | | |
| NDUFV1 | 2 | (intron 6 160) | tgtgccggccccagccctga C/G catgcatccctttggggacc | 7000 | | |
| NDUFV1 | 3 | (intron 9 27) | accacccttctgcgtagcac G/A gagggtgggtggcatcaagg | 7001 | | |
| NDUFV1 | 4 | (3′flanking region 1111) | tgtaggctgaggtcagcccc A/C atccagtccaaagcccaccc | 7002 | | |
| NDUFV1 | 5 | (3′flanking region 1658) | gaatgcggaagtgctctgtg G/A gcacccaccatgctccgggc | 7003 | | |
| NDUFV1 | 6 | (3′flanking region 1713) | gatctggggcggagggtaca C/T ggggctggcgctgggtgaag | 7004 | | |
| NDUFV1 | 7 | (intron 4 214) | tggtgtaaattttttttttt T/Δ gcttcaaaaatatagtattt | 7005 | | |
| NDUFV1 | 8 | (3′flanking region 772-7 | tgaactcggggttcagggtc TTC/Δ ctgtgaacactggttttcaa | 7006 | | |
| NDUFV2 | 1 | (intron 1 526) | ggaaatgctggctaaataaa C/T ggtatcaaactaactctgaa | 7007 | | |
| NDUFV2 | 2 | (intron 1 6689) | tcgttggatggtagtattgt T/G tgaacaacagaagaaattca | 7008 | | |
| NDUFV2 | 3 | (intron 1 14767) | ccaaatgcatgccagcagag C/T gtggcaggaaggtacacaag | 7009 | | |
| NDUFV2 | 4 | (coding region (Ala29Val)) | aaggaatttgcataagacag T/C tatgcaaaatggagctggag | 7010 | | |
| NDUFV2 | 5 | (intron 2 − 289) | cagaagatcttactctctaa T/G gaagctggataacacttttt | 7011 | | |
| NDUFV2 | 6 | (intron 2 − 168) | tttactttggtaatcatact T/C atcaaatgtgtgtttagaca | 7012 | | |
| NDUFV2 | 7 | (intron 4 677) | aeaccacatactatttgatt C/A tgatgagaatcacataacca | 7013 | | |
| NDUFV2 | 8 | (intron 4 2295) | tatgattcaactttcaaaag A/T gtattgtgatatgaaataga | 7014 | | |
| NDUFV2 | 9 | (intron 5 102) | caacttctgccatcttattg G/A atctgtacttacctagtaat | 7015 | | |
| NDUFV2 | 10 | (intron 7 5466) | tggtaagaggctttaagata A/C caaatgctcagctttcagga | 7016 | | |
| NDUFV2 | 11 | (intron 1 13562-13563) | tactcttaaaattaatcctt (CTT) ttattataagtatacagtct | 7017 | | |
| NDUFV2 | 11 | (intron 1 13562-13563) | tactcttaaaattaatcctt ttattataagtatacagtc | 7018 | | |
| NDUFV3 | 1 | (5′flanking region − 222) | cgccgcgcccccgccacagc G/A cccaggcgcccgcagggcac | 7019 | | |
| NDUFV3 | 2 | (5′flanking region − 111) | tggccccaagggaggcactt A/G gccctactggggatgcgcgc | 7020 | | |
| NDUFV3 | 3 | (intron 1 137) | ttgggccgctgaccccgctc C/T ctgggcccaggactgaccgc | 7021 | | |
| NDUFV3 | 4 | (intron 2 152) | tatacaagacacaagatcta T/C aacagattttagaccaaaca | 7022 | | |
| NDUFV3 | 5 | (intron 2 6304) | ttcacacatgaaggggttcc G/A aaatttttgtcaagaaagac | 7023 | | |
| NDUFV3 | 6 | (intron 2 6433) | tcgccttcgtcttcatcctc T/G tccagctcctctgattctga | 7024 | | |
| NDUFV3 | 7 | (intron 2 6563) | cctttgaaaacagagccccc C/T gagttacagtatcagcaaaa | 7025 | | |
| NDUFV3 | 8 | (intron 2 9619) | actatcttctgtgcgcatgc G/A cagagcccaccttgcagagc | 7026 | | |
| NDUFV3 | 9 | (intron 2 9858) | aggatgccagctctttaaat G/A agacatcgtttttgcttaac | 7027 | | |
| NDUFV3 | 10 | (intron 2 11673) | cttggtaggtaagcgcctgt A/G tgtgagccaagtcattcata | 7028 | | |
| GGT1 | 1 | intron 1 + 85 | ttatccagtaaggtggctcc G/A tcacctcttttcctggtggg | 7029 | | |
| GGT1 | 2 | exon 3 + 68 | gacggccaggtccggatggt G/T gtgggagctgctgggggcac | 7030 | | |
| TGM1 | 1 | exon 2 + 179 | tgccgaaatgcggcagatga C/T gactggggacctgaaccctc | 7031 | | |
| TGM1 | 2 | intron 9 + 1594 | acttaccactctgtcctctc C/T tgccaggcctcttcctgtca | 7032 | | |
| TGM1 | 3 | intron 9 + 1933 | ccgcacctctgtaccctgcc C/G ccatcctccagcagagcagc | 7033 | | |
| TGM1 | 4 | intron 10 + 54 | tcagtcatgggttctctggt C/T ccaacttcaccgctgactga | 7034 | | |
| TGM1 | 5 | intron 10 + 420 | aggaggccgggagtcaggcc A/G ccctcagaccctctggctca | 7035 | | |
| TGM1 | 6 | intron 12 + 101 | gggagtccctgggggaagcc T/G catgtagggaagcaggcctc | 7036 | | |
| TGM1 | 7 | intron 13 + 72 | ggataaggacatcagaggtg G/A gcgctaagccagcagcaggc | 7037 | | |
| TGM1 | 8 | intron 14 + 1671 | atctcttacccacaccccca C/G catggtggggaggttcctca | 7038 | | |
| TGM1 | 9 | intron 14 + 1691 | ccatggtggggaggttcctc G/A tcctaagggatccgcagagc | 7039 | | |
| TGM1 | 10 | intron 14 + 2983 | tccctgcctccctccttcag G/A gagctcagaaacaccttcaa | 7040 | | |
| TGM1 | 11 | intron 14 + 3158 | ggaaacccctcagaaccagg T/C tccaagccaaatgctttgcc | 7041 | | |
| TGM1 | 12 | intron 14 + 3816 | cagaatacaaaagtgggatg G/C gaggcaaggagtcccgttag | 7042 | | |
| TGM1 | 13 | exon 15 + 233 | ctcgaggtggagcttagccc T/C gtgccaggagcaatgggact | 7043 | | |
| TGM1 | 14 | exon 10 + 369 | ggagtcagtcttcacttgca C/A tgggggaacagatgctaata | 7044 | | |
| CYP1A1 | 1 | 5′flanking − 1061 | ccgccccgactccctccccc C/G tcgcgtgactgcgagccccc | 7045 | | |
| CYP1A1 | 2 | 5′flanking − 1035 | tgactgcgagcccccgcgcc G/A ggccggggaatgggtcggct | 7046 | | |
| CYP1A1 | 3 | 5′flanking − 1020 | gcgccgggccggggaatggg T/G cggctgggtggctgcgcggg | 7047 | | |
| CYP1A1 | 4 | 5′flanking − 947 | cgcgcctccgggccaggtgg G/A gcggggacgggccgcctgac | 7048 | | |
| CYP1A1 | 5 | intron 1 + (1326-1334) | cattcattgagaattgagcc (A)8-9 ccctggcctggatttctctg | 7049 | | |
| CYP1A1 | 6 | intron 1 − 1357 | ctggcctggatttctctgac T/C aaagagctcaatctagctgg | 7050 | | |
| CYP1A1 | 7 | intron 1 − 1590 | ccactcttcaaaaggaggta C/T atgtgacagcagctggaaat | 7051 | | |
| CYP1A1 | 8 | exon 2 + 160 | gaatccaccagggccatggg G/A ctggcctctgattgggcaca | 7052 | | |
| CYP1A1 | 9 | 3′flanking + (710-720) | caccgcgagatttccaggtc (T)10-12 gagacggagtctcactgtgt | 7053 | | |
| CYP1A1 | 10 | 3′flanking + 834 | gcctcagcctcccaagtagc C/T gggactacaggcgcctgcca | 7054 | | |
| CYP1A2 | 1 | intron 1 + 103 | gcctgggctaggtgtagggg T/G cctgagttccgggctttgct | 7055 | | |
| CYP1A2 | 2 | intron 2 + 371 | cttccctgtgttcacactaa C/T cttttccttctttgaaattg | 7056 | | |
| CYP1A2 | 3 | intron 4 + 44 | atagccaggagaagccttga G/A acccaggttgtttgttcagt | 7057 | | |
| CYP1A2 | 4 | intron 4 + 206 | aagagtgacatggggtataa G/C aggggataattcatggggca | 7058 | | |
| CYP1A2 | 5 | intron 5 + (623-648) | tgccccaggctgcctgctgc (T)22-25 catagaaaatagaaaaacat | 7059 | | |
| CYP1A2 | 6 | intron 6 + 81 | tccctgctaggaactgttta T/C ataatgaaaggaggggacct | 7060 | | |
| CYP1A2 | 7 | exon 7 + 101 | ctggccatcctgctacagca A/T ctggagttcagcgtgccgcc | 7061 | | |
| CYP1A2 | 8 | exon 7 + 295 | cggctgcctgtctccatcaa C/T tgaagaagacaccaccattc | 7062 | | |
| CYP1B1 | 1 | 5′flanking − 3669 | tgtatcctgtgaagcatcac G/A gttatccttctctgcacatg | 7063 | | |
| CYP1B1 | 2 | flanking − 3149 | tgacagcacttaccaaccta G/C ttcctctgatttttgagtca | 7064 | | |
| CYP1B1 | 3 | flanking − 1222 | gggggaagccacccccgccc G/A agcgcctccggcttccctta | 7065 | | |
| CYP1B1 | 4 | flanking − 376 | ttccgggaagcaagctcaag T/C cgcggagagggaagggaggt | 7066 | | |
| CYP1B1 | 5 | flanking − 265 | ctggggacaccgtgcggcct C/T gattggaggtggctgtgatg | 7067 | | |
| CYP1B1 | 6 | intron 1 + 129 | tgcccgcagcgttgtcccca G/A attgcaggaaccgttacgcg | 7068 | | |
| CYP1B1 | 7 | intron 1 + 379 | tgagtgtcacgccttctcct C/T tctgtccccagcatgggcac | 7069 | | |
| CYP1B1 | 8 | exon 3 + (799-800) | agcttctgggagattttttt (T) gagtcaaagacttaaagggc | 7070 | | |
| CYP1B1 | 8 | exon 3 + (799-800) | agcttctgggagattttttt gagtcaaagacttaaagggc | 7071 | | |
| CYP1B1 | 9 | exon 3 + 1284 | agtatagtggggttccatga G/T ttatcatgaattttaaagta | 7072 | | |
| CYP1B1 | 10 | exon 3 + 1398 | tcagcaaagaaaaaaaaaaa A/Δ gccagccaagctttaaatta | 7073 | | |
| CYP1B1 | 11 | exon 3 + 1468 | tctcataggttaaaaaaaaa A/Δ gtcaccaaatagtgtgaaat | 7074 | | |
| CYP1B1 | 12 | exon 3 + 1564 | ttgaataatatatgccttgt G/A taatattgaaaattgaaaag | 7075 | | |
| CYP1B1 | 13 | exon 3 + 1762 | ttgaaattctatttataata C/Δ agaatcttgttttgaaaata | 7076 | | |
| CYP1B1 | 14 | 3′flanking + (2216-2226) | agccagcctctttctttttc (T)10-12 aaaatttattcctatttcct | 7077 | | |
| CYP1B1 | 15 | 3′flanking + 2230 | tttttctttttttttttaaa A/Δ tttattcctatttccttaca | 7078 | | |
| CYP3A4 | 1 | intron 2 + (754-763) | cacaaaatgaggttgtgggg (T)9-11 acacaaaggcggaatcacat | 7079 | | |
| CYP3A4 | 2 | intron 7 + 258 | accactaataaactttctgc C/T tctatggatttgcctattct | 7080 | | |
| CYP3A4 | 3 | intron 7 + 894 | tgctgatctcactgctgtag C/T ggtgctccttatgcatagac | 7081 | | |
| CYP3A4 | 4 | exon 9 + (32-33) | ttccttcagctgatgattga (A) ctctcagaattcaaaagaaa | 7082 | | |
| CYP3A4 | 4 | exon 9 + (32-33) | ttccttcagctgatgattga ctctcagaattcaaaagaaa | 7083 | | |
| CYP3A4 | 5 | intron 10 + 12 | cccaataaggtgagtggatg G/A tacatggagaaggagggagg | 7084 | | |
| CYP3A4 | 6 | intron 10 + 459 | agacatctgacttttttttt T/Δ gaaaggtaacaatcactttc | 7085 | | |
| CYP3A4 | 7 | intron 10 + 608 | agccgtctcgaatgtctccc C/T acttcataactcctccacac | 7086 | | |
| CYP3A4 | 8 | intron 12 + 2467 | ttttttgcccattactccat A/G gagatcagaatatcactctg | 7087 | | |
| CYP3A5 | 1 | exon 1 + 69 | ggaagactcacagaacacag T/C tgaagaaggaaagtggcgat | 7088 | | |
| CYP3A5 | 2 | intron 1 + (955-956) | tgtgggtagtggaggctcca (A) cctgtcccattaacttctac | 7089 | | |
| CYP3A5 | 2 | intron 1 + (955-956) | tgtgggtagtggaggctcca cctgtcccattaacttctac | 7090 | | |
| CYP3A5 | 3 | intron 1 + 1126 | acatttttaaatgaattgat A/G tggtttaaattcattcattt | 7091 | | |
| CYP3A5 | 4 | intron 1 + 1145 | tatggtttaaattcattcat T/G tttaaaccacaattttttgg | 7092 | | |
| CYP3A5 | 5 | intron 1 + 1543 | ttcatgcgtcctggccccac C/A gtggaggtcactcaaagggc | 7093 | | |
| CYP3A5 | 6 | intron 1 + 2366 | cttatcttatatgccatact G/A caccatttgctatcaacagg | 7094 | | |
| CYP3A5 | 7 | intron 4 + 1813 | tggttctaattttactcttc G/A tgttcttcatccttgaaaat | 7095 | | |
| CYP3A5 | 8 | intron 4 + 1887 | aatgacatgaacaaggtgtg A/T ttgtgaagcaagggatattt | 7096 | | |
| CYP3A5 | 9 | intron 4 + 3384 | gagtgcttcgctatttgcct C/T aacaagaaaaagtcatttgt | 7097 | | |
| CYP3A5 | 10 | intron 4 + 3415 | agtcatttgtccacttttca T/C tgaacaatcttccttcatcc | 7098 | | |
| CYP3A5 | 11 | intron 4 + 3760 | aagataacacactggaagtc G/A cacaccaccataaaactgaa | 7099 | | |
| CYP3A5 | 12 | intron 4 + 3885 | acaattcacttcacgtggca C/T tgcaatagcgtcctctcgct | 7100 | | |
| CYP3A5 | 13 | intron 4 + 5061 | tacctacttttcaaaaaaaa A/Δ tcaccacatcatggcatccc | 7101 | | |
| CYP3A5 | 14 | intron 4 + 5316 | ccagatggctgggtctcccc A/T ctcccacccccgccccacat | 7102 | | |
| CYP3A5 | 15 | intron 9 + 77 | gttctgaaaatgtgcacgaa G/T tattccaggaagatgagaat | 7103 | | |
| CYP3A5 | 16 | intron 9 + 1791 | aaatttttattgggaaaaag C/T ctaccccatatttacttaca | 7104 | | |
| CYP3A5 | 17 | intron 12 + 1408 | atttaaataaaaaaaaaaaa A/Δ cacgagtccacaagaatttg | 7105 | | |
| CYP3A5 | 18 | 3′flanking + 542 | tggagaaaatattcatagtt T/C cattctgccttctttgaaga | 7106 | | |
| CYP3A5 | 19 | 3′flanking + 737 | atgaacactgaataaaaaat T/G gtcaattcgtcagttgattg | 7107 | | |
| CYP3A5 | 20 | 3′flanking + 804 | ttttccttttttattctttc A/C ttttcactccttttctgaat | 7108 | | |
| CYP3A7 | 1 | 5′flanking − 1680 | cccaaggaacatgtggctcc C/A ggcacatacctggcacaaca | 7109 | | |
| CYP3A7 | 2 | 5′flanking − 1191 | tagaaaatcctccacttgtc A/C aaaaggaagccatttgcttt | 7110 | | |
| CYP3A7 | 3 | intron 1 + 1173 | cccccatttcaaatacacct G/A cttagcaggttatcctaaac | 7111 | | |
| CYP3A7 | 4 | intron 1 + 1597 | tttttctgttagcctcttca T/C tgtaaccaaaagcagcatta | 7112 | | |
| CYP3A7 | 5 | intron 3 + 762 | tccagtgtctgcctattccc T/C tcttctttttttcttccctt | 7113 | | |
| CYP3A7 | 6 | intron 7 + (1060-1069) | atggtttcgttttctgttgg (T)9-10 ctacagaagtctttccattc | 7114 | | |
| CYP3A7 | 7 | intron 11 + (592-594) | taagacaaggtagggaggag AAG/Δ gaggagaattagaaaaacaa | 7115 | | |
| CYP3A7 | 8 | intron 12 + 911 | ccccctccattaacaatatc C/T tctcattttattccatttaa | 7116 | | |
| CYP3A7 | 9 | intron 12 + 1137 | gtctgtctgcagggaaaata T/Δ attcatgccttttgaaaatt | 7117 | | |
| CYP3A7 | 10 | intron 12 + 2147 | tattgtcagtaatttttttt T/Δ actttgatgctatactttct | 7118 | | |
| CYP3A7 | 11 | exon 13 + 218 | ttcatccaatgtgctgcata A/C ataatcaggcattctgtacg | 7119 | | |
| CYP3A43 | 1 | intron 1 + 3579 | tcatgctcactttttttttt T/Δ ctcaaaatgatcagtcacac | 7120 | | |
| CYP3A43 | 2 | intron 2 + 2427 | tagagggaatcttttttttt T/Δ cctttttttctctgccccag | 7121 | | |
| CYP3A43 | 3 | intron 3 + 3034 | tttttatatagctagggaga T/C tgtaaattaacaagtttcct | 7122 | | |
| CYP3A43 | 4 | intron 3 + 3433 | agtcaagataactttttttt T/Δ cataaaggaccacagtatgt | 7123 | | |
| CYP3A43 | 5 | intron 3 + 3504 | catgactcagtttccaacca T/C aacttttcattttggcatag | 7124 | | |
| CYP3A43 | 6 | intron 4 + 2767 | tagtgacttttgaaaaaaaa A/Δ ttagtaataagcaaaagcct | 7125 | | |
| CYP3A43 | 7 | exon 5 + 22 | aaaacttaaggcacttttca G/A aaatcccattggacctaaag | 7126 | | |
| CYP3A43 | 8 | intron 12 + (1585-1584) | tactttgagccctcattctc (A) ccaagtcacttcagtgtcag | 7127 | | |
| CYP3A43 | 8 | intron 12 + (1585-1584) | tactttgagccctcattctc ccaagtcacttcagtgtcag | 7128 | | |
| CYP4B1 | 1 | 5′flanking − 333 | gaaacattcacagtgcttgt A/T tgagaagacagtggttatta | 7129 | | |
| CYP4B1 | 2 | 5′flanking − 18 | gagcagctgaaggcaggtca G/T atgaaggctaggtggctgga | 7130 | | |
| CYP4B1 | 3 | intron 1 + 341 | tccaaaacctctggatagta C/T atagaagtaggcaatccatt | 7131 | | |
| CYP4B1 | 4 | intron 1 + 542 | cctatgcgtggctcaggagc C/T gtgacaccttcccaggttca | 7132 | | |
| CYP4B1 | 5 | intron 1 + 2856 | gaggactttgcacatagtag G/A tgctcagctatattgttagc | 7133 | | |
| CYP4B1 | 6 | intron 1 + (2923-2938) | caacaaattggtgtgtgtgg (GT)7-8 agaatgccagctcccagatc | 7134 | | |
| CYP4B1 | 7 | intron 1 + 6086 | tttggaatctaaagactggg G/T cacgatgctagttgtgtgac | 7135 | | |
| CYP4B1 | 8 | intron 1 + 6598 | ttttggggtgtggggagagg G/A cccatagtagggagacagct | 7136 | | |
| CYP4B1 | 9 | intron 1 + 6660 | acctaagggtgtccatcctg A/G aggagagcagtcctaggggg | 7137 | | |
| CYP4B1 | 10 | intron 1 + 7242 | ccctggtctcccttaactca T/C gctggactgttccctttgct | 7138 | | |
| CYP4B1 | 11 | intron 2 + 107 | gcctgtgtactaagtctgcg C/G agctgaggttcccaccctac | 7139 | | |
| CYP4B1 | 12 | intron 3 + 361 | atggtgtggtggtaggacca C/T ggctggtcaccagaggctgt | 7140 | | |
| CYP4B1 | 13 | intron 4 − 492 | aaaggctttcacatctaaaa C/A gtgtctcctcattttctgtc | 7141 | | |
| CYP4B1 | 14 | intron 4 − 315 | ggattacttacatatacacc A/G tgcgggggagctcaccacct | 7142 | | |
| CYP4B1 | 15 | intron 4 − 157 | ctacccaccctatcctgata T/C tccagcaggatggagggcag | 7143 | | |
| CYP4B1 | 16 | exon 5 + 22 | acaagtgggaagagaaagct C/T gggagggtaagtcctttgac | 7144 | | |
| CYP4B1 | 17 | intron 5 + 125 | cccagggagccttagcttgc G/A gggagacaggacctgctcat | 7145 | | |
| CYP4B1 | 18 | intron 5 + (267-289) | tgtctaagccaatccctcct CCT/Δ accctctgcttaacagggac | 7146 | | |
| CYP4B1 | 19 | intron 6 + 54 | gcctgggttcctcctcctgg C/T ccctctatgccccctcccat | 7147 | | |
| CYP4B1 | 20 | intron 7 + (99-100) | agctcttaagcatttccccc (TC) tttcctcagcaaatataacc | 7148 | | |
| CYP4B1 | 20 | intron 7 + (99-100) | agctcttaagcatttccccc tttcctcagcaaatataacc | 7149 | | |
| CYP4B1 | 21 | intron 8 + 114 | tcctggtttctctactgcat G/A gccctgtaccctgagcacca | 7250 | | |
| CYP4B1 | 22 | exon 8 + 139 | tgtaccctgagcaccagcat C/T gttgtagagaggaggtccgc | 7151 | | |
| CYP4B1 | 23 | intron 8 + 247 | agaaagttgtcaacaagagg C/T tgatattttgtgtgctaact | 7152 | | |
| CYP4B1 | 24 | intron 8 + 366 | tgtgggggtgaacagagctg A/G gacagctgggagagccagtt | 7153 | | |
| CYP4B1 | 25 | intron 8 + 650 | cctttgcttgtggtcagaca C/A cctgcctttctctctgggct | 7154 | | |
| CYP4B1 | 26 | intron 8 + 844 | tcatatgtgagaatcccccc C/A ccacggggtatccagacaca | 7155 | | |
| CYP4B1 | 27 | intron 8 + 1767 | tcccattccaagaatgttct G/T gttgtgttgctggcagggat | 7156 | | |
| CYP4B1 | 28 | exon 9 + 53 | tgtgcatcaaggagagcttc C/T gcctctacccacctgtgccc | 7157 | | |
| CYP4B1 | 29 | intron 9 + 652 | agtcggatgtggtcatgaac G/T ctctgtcactggcagtggtc | 7158 | | |
| CYP4B1 | 30 | intron 9 + 774 | cctggtcaccaacctctgtt C/T tgcccacaggaagcctgatc | 7159 | | |
| CYP4B1 | 31 | intron 10 + 33 | tgggctgggagatcagacag G/T gtgggggantgggacggtca | 7160 | | |
| CYP4B1 | 32 | exon 12 + 224 | ccagatggctcaggctgtga C/A ctccctgggcaccaccctcc | 7161 | | |
| CYP4B1 | 33 | exon 12 + 270 | ctgggtgtggaggagttggg G/A ccccctgccttcaggagctg | 7162 | | |
| CYP4B1 | 34 | 3′flanking + 129 | tctgtgtctcacagtcacgt G/A gtgctccaggcattcagggt | 7163 | | |
| CYP4B1 | 1 | intron 1 + (145-146) | ccaagcccctggcaacctca CA/Δ gtgattcaggctgcgccttt | 7164 | | |
| CYP4B1 | 2 | intron 1 + 193 | tttaatcagtctctctctct C/T tttcccattctaagtgctta | 7165 | | |
| CYP4B1 | 3 | intron 1 + 324 | ccctgctctacctccggcac T/C gcccgtccctgcctctccac | 7166 | | |
| CYP4B1 | 4 | intron 1 + 367 | tccctgcaggtccctgggcc G/C ttctctgggcctcaggatct | 7167 | | |
| CYP4B1 | 5 | intron 1 + 402 | ggatctcaccgtccatcccg T/C ctgccctgcaggatctccca | 7168 | | |
| CYP4B1 | 6 | exon 2 + 35 | ccctgtcctgcctgggcctc T/G ggccagtggcagcatcccct | 7169 | | |
| CYP4B1 | 7 | exon 2 + 166 | cggtgtttcccacaaccccc A/G agacggaactggttttgggg | 7170 | | |
| CYP4B1 | 8 | intron 2 + 125 | ggcagagaagcagaggaggc A/G tcttactcattcctctgctt | 7171 | | |
| CYP4B1 | 9 | intron 2 + 440 | gggccgtctcccacttccac T/C acacccgaaggcacctttct | 7172 | | |
| CYP4B1 | 10 | exon 3 + 48 | gttctgactcagctggtggc C/T acctacccccagggctttaa | 7173 | | |
| CYP4B1 | 11 | intron 3 + 701 | agactccaccccagcttggg T/A ccctttccttgacccctgtg | 7174 | | |
| CYP4B1 | 12 | intron 3 + 742 | cttcccatcgttggacgggc G/A aggctgagcagggggaatgg | 7175 | | |
| CYP4B1 | 13 | intron 3 + 1020 | gctttagctttctccatgtc G/A cttttcctatcaaggtggcc | 7176 | | |
| CYP4B1 | 14 | intron 3 + 1039 | cgcttttcctatcaagctgg C/A cttttcctcatgatgtcaac | 7177 | | |
| CYP4B1 | 15 | intron 3 + 1040 | gcttttcctatcaaggtggc C/G ttttcctcatgatgtcaacg | 7178 | | |
| CYP4B1 | 16 | intron 3 + 1920 | ccacctgtctaacctctgtt G/C ctgtttgctcatgtctgggg | 7179 | | |
| CYP4B1 | 17 | intron 3 + 1945 | ttgctcatgtctggggcgtg T/A ctctacaatggctgttatat | 7180 | | |
| CYP4B1 | 18 | intron 3 + 2621 | agcattctgtagaatgctga G/A ctgtgctcaggggttgcgga | 7181 | | |
| CYP4B1 | 19 | intron 3 + 2665 | tgttggatcgtgtaggaggc A/G tgtcaaggcatgctggaacc | 7182 | | |
| CYP4B1 | 20 | intron 6 + 194 | gggtttgaactggtgggtgt G/T ctcagagctctgtaggggac | 7183 | | |
| CYP4B1 | 21 | intron 7 + 67 | tgtgaaatgtcagatgaaag G/A acttgaacttgattaagagg | 7184 | | |
| CYP4B1 | 22 | intron 7 + 2811 | ttccaagggaaattgccatt T/G aattctcctgtaactcaggt | 7185 | | |
| CYP4B1 | 23 | intron 7 + (3096-3097) | ggggtgcgggttgggggggg (G) ttactgccttctctccagga | 7186 | | |
| CYP4B1 | 23 | intron 7 + (3056-3057) | ggcgtgggggttgggggggg ttactgccttctctccagga | 7187 | | |
| CYP4B1 | 24 | intron 8 + 145 | ggtgctgtctaccttcgggt G/A ctgaagcagcccagagaccc | 7188 | | |
| CYP4B1 | 25 | exon 9 + 44 | ctctcctgggtcctgtacca C/T cttgcaaagcacccagaata | 7189 | | |
| CYP4B1 | 26 | exon 11 + 48 | gaacccatcacaacccagct G/A tgtggccggaccctgaggtg | 7190 | | |
| CYP4B1 | 27 | intron 12 '108 | tggtccaagttccagctctc C/T ttccctcacctcctctggag | 7191 | | |
| CYP4B1 | 28 | intron 12 + 285 | gcatggggatccaggcacgg A/T tacccccttctctattcctc | 7192 | | |
| CYP4B1 | 29 | exon 13 + 238 | aagtgaagcctagaattacc C/A taagaccctgttccacagtc | 7193 | | |
| CYP4B1 | 30 | exon 13 + 342 | tgtgcgtgaatgttcatggc G/A gccctattcacagtagccaa | 7194 | | |
| CYP4B1 | 31 | exon 13 + 863 | tagtgtactgtccttttata T/C gaaatttccagaacaggcca | 7195 | | |
| CYP4B1 | 32 | exon 13 + 707 | aaatgttccggacctagata G/C tgacgaaggtagcacgacac | 7196 | | |
| CYP4F3 | 1 | intron 2 + 258 | cattaatgcacctctgcggg G/T ctcttgggcagggggttggg | 7197 | | |
| CYP4F3 | 2 | intron 2 + 916 | ttagggacatgtcctgagtc C/T acactgctccccacaaacct | 7196 | | |
| CYP4F3 | 3 | intron 2 + 3417 | atccaggtctcacacagtgt C/T acttcctctcttggctttag | 7198 | | |
| CYP4F3 | 4 | intron 2 + 4090 | gagagcatgaattgggtcct G/A tgtctttctctccagattca | 7200 | | |
| CYP4F3 | 5 | intron 3 + 89 | tctgctgcctccagcgggtc G/A cgtgcccatgtgcagacagg | 7201 | | |
| CYP4F3 | 6 | intron 3 + 243 | tcaagtctgctgtacggcta C/T gtcttgtcacctgtatattt | 7202 | | |
| CYP4F3 | 7 | intron 3 + 502 | aggtctgggacccagggtcc G/C taagtgaactgtctgagaca | 7203 | | |
| CYP4F3 | 8 | intron 3 + 755 | ttttgtggccatgtcaggac A/T tgtgaacacatgtcagtgtc | 7204 | | |
| CYP4F3 | 9 | intron 3 + 855 | gggacagacagggtgtccta G/A gcccttgtgaaggcattctg | 7205 | | |
| CYP4F3 | 10 | intron 3 + 970 | cctgacatagctcctacgtg C/T catgttaggcagtgtcattg | 7206 | | |
| CYP4F3 | 11 | intron 6 + 122 | gaggagttgttatacctgat C/T gttgaaggactggtatgaat | 7207 | | |
| CYP4F3 | 12 | exon 7 + 159 | ggtgcacgacttcacagatg C/A cgtcatccaggagcggcgcc | 7208 | | |
| CYP4F3 | 13 | intron 7 + 2107 | caggttgccagtgatttttt T/Δ ctcagaaagttttcatcaag | 7209 | | |
| CYP4F3 | 14 | intron 7 + 2285 | gaccaagaagggtctaggag T/A gcaagatgggcttgggtttc | 7210 | | |
| CYP4F3 | 15 | intron 8 + 132 | cctcaatgcaaggttgctgt A/C caccctcgggtgctgaagca | 7211 | | |
| CYP4F3 | 16 | exon 9 + 59 | taccaccttgcaaagcaccc G/A gaataccaggagcgctgtcg | 7212 | | |
| CYP4F3 | 17 | intron 9 + 13 | attgaatggtgagtgcaggt G/A ctggtgccctgttcctgagc | 7213 | | |
| CYP4F3 | 18 | intron 9 + 36 | ggtgccctgttcctgagcct G/C tctcattggctctgttcccc | 7214 | | |
| CYP4F3 | 19 | intron 9 + 167 | acccatcctgactgtctggg C/G aaaggttataggcccttagg | 7215 | | |
| CYP4F3 | 20 | intron 9 + 369 | tccctaattcctacccttcc G/A tccagtccagggatttataa | 7216 | | |
| CYP4F3 | 21 | intron 9 + 458 | tcattcatccatccagtcat T/C gttcagcaaatactctcata | 7217 | | |
| CYP4F3 | 22 | intron 10 + 46 | ctcctgggtaggaagagggg A/C ccctcaggcagggagcattg | 7218 | | |
| CYP4F3 | 23 | intron 10 + 63 | gggcccctcaggcagggagc C/A ttgtcctgactgcccccttc | 7219 | | |
| CYP4F3 | 24 | intron 11 + 14 | ccctgaggtgcgggcccccc C/G tctctgtttttgtccattcc | 7220 | | |
| CYP4F3 | 25 | intron 11 + 84 | gatcaggagaatccaacatc G/A cctccctccaagacacacac | 7221 | | |
| CYP4F3 | 26 | intron 11 + 113 | caagacacacaccactgtct T/C tccaaggctgccggactggg | 7222 | | |
| CYP4F3 | 27 | intron 11 + 164 | cggcaacccttcttggtctc T/G cctccaggtctatgacccct | 7223 | | |
| CYP4F3 | 28 | intron 11 + 168 | ggcaacccttcttggtctcg T/C ctccaggtctatgacccctt | 7224 | | |
| CYP4F3 | 29 | intron 12 + 156 | gaaaaggcccacagagtagg G/A ttgggttggtcctagaagga | 7225 | | |
| CYP4F3 | 30 | intron 12 + 253 | gagctcggctaggctcgcag T/G atatgcaagcccacatgggg | 7226 | | |
| CYP4F3 | 31 | intron 12 + 346 | tggcggccccaggccaggcc A/C ccggcgggatggggccagga | 7227 | | |
| CYP4F8 | 1 | 5′flanking − 61 | accatgtttacccatcattg G/T tcctggagctccccagcccc | 7228 | | |
| CYP4F8 | 2 | exon 1 + 67 | gtggcagcatccccgtggct G/T ctcctgctggtggtcggggc | 7229 | | |
| CYP4F8 | 3 | intron 1 + 707 | tacgcagcaggtattcacca T/G tatttccacattatccactg | 7230 | | |
| CYP4F8 | 4 | intron 1 + 857 | acaccccctaccctcacatc G/A tgacacagctgggccagaag | 7231 | | |
| CYP4F8 | 5 | intron 1 + 907 | tgccatctccaccctccccc G/A tgcaggggcatcttctttat | 7232 | | |
| CYP4F8 | 6 | intron 2 + 668 | tgtggcacttccaccatatg T/C tcattgccctcttgctccag | 7233 | | |
| CYP4F8 | 7 | intron 2 + 818 | gccacagagaccatggctca G/A gccccaaaatgctgagtgac | 7234 | | |
| CYP4F8 | 8 | intron 2 + 1079 | tatgcttgggtgttgcagaa C/T atgttggaccatgtaggagc | 7235 | | |
| CYP4F8 | 9 | intron 2 + 1194 | ccggtcccctttatgccccc C/A accctcctttcttcttctgc | 7236 | | |
| CYP4F8 | 10 | intron 5 + 45 | aacatgggatggagtggggg G/T gtgggtgtggggagagcaaa | 7237 | | |
| CYP4F8 | 11 | exon 8 + (19-20) | ggccatgacaccacggccag (GCCAG) tggccgctccggggccttgt | 7238 | | |
| CYP4F8 | 11 | exon 8 + (19-20) | ggccatgacaccacggccag tggcctctcctgggtcttgt | 7239 | | |
| CYP4F8 | 12 | intron 8 + 222 | tttatttccccactaacttg C/G tatgcaagcttagtaaaatc | 7240 | | |
| CYP4F8 | 13 | intron 8 + 334 | cttggagaattaacggcaaa A/T accgcaatgacttttggacc | 7241 | | |
| CYP4F8 | 14 | intron 8 + 1999 | ttctaagcacatttactctc T/C cgctcttagctatgatctag | 7242 | | |
| CYP4F8 | 15 | intron 8 + 4184 | caggagggccgcgtatgctc C/T cgggacaattgttgggtgtt | 7243 | | |
| CYP4F8 | 16 | exon 9 + 119 | acgtggtgctcccagacagc C/T gagtcatccccaaaggtgcc | 7244 | | |
| CYP4F8 | 17 | intron 11 + 282 | gggttgggggttccgggcct G/C gttcctggcgcagtggggcc | 7245 | | |
| CYP4F8 | 18 | intron 11 + 340 | tgcagtcagaccttccacct C/T ggcccccaggaactgcatcg | 7246 | | |
| CYP4F8 | 19 | 3′flanking + 35 | atcacctacctttgcaccaa T/C taccttttcagacccccggt | 7247 | | |
| CYP4F8 | 20 | 3′flanking + 83 | ctgtgttggcccctgtgcct G/C agtcccgcggatggccagta | 7248 | | |
| CYP4F8 | 21 | 3′flanking + 90 | ggcccctgtgcctcagtccc A/G cggatggccagcagggggcg | 7249 | | |
| CYP27A1 | 1 | intron 1 + 295 | aggagggagctgtcttggga A/G gagagtggcagaggcaaatg | 7250 | | |
| CYP27A1 | 2 | intron 1 + 17503 | cagtgcataaagcctctgat C/T ctccttagagaaggagggac | 7251 | | |
| CYP27B1 | 1 | intron 6 + 173 | cagcccctagcctcatcttg C/T tgtctccattttgtgctttg | 7252 | | |
| CYP27B1 | 2 | intron 8 + 113 | atataagacctggtagaatg A/C atcttctgaaatatgataag | 7253 | | |
| CYP27B1 | 3 | 3′flanking + 1081 | taccctggaatcagtgatga G/C aattctgcccatccgtactc | 7254 | | |
| AADAC | 1 | exon 1 + 29 | attaaagtacactattcagg C/T atatcatgtaggtttactct | 7255 | | |
| AADAC | 2 | intron 1 + 138 | gcggtggcctttgacaatgt G/A ttacttagaaatgttgtttg | 7256 | | |
| AADAC | 3 | intron 1 + 142 | tggcctttgacaatgtgtta C/T ttagaaatgttgtttgtttt | 7257 | | |
| AADAC | 4 | intron 1 + 1033 | ttccagcagagacaccaaca A/G gtaaaaacaccccagctaca | 7258 | | |
| AADAC | 5 | intron 1 + 1253 | tttttttccctcatatttgc T/C gtctgtgctacaatatgtga | 7259 | | |
| AADAC | 6 | intron 1 + 1366 | ctctggtagccttttaatta A/G ttaattcattcatttactta | 7260 | | |
| AADAC | 7 | intron 1 + 1369 | cggtagccttttaattaatt A/C attcattcatttacttacat | 7261 | | |
| AADAC | 8 | intron 1 + 2501 | ggttacagaaagaatggtgg C/A ttggccaaaaaatgataggg | 7262 | | |
| AADAC | 9 | intron 2 + 46 | tgtcactgaggtagttcgca A/G acattttactaagtcttcag | 7263 | | |
| AADAC | 10 | intron 2 + 1971 | aaatgagagttaagtaggag A/C attttcttttatttttgtgc | 7264 | | |
| AADAC | 11 | intron 2 + 1988 | gagaattttctttcatcttt A/G tgcaggagaaatacaaacaa | 7265 | | |
| AADAC | 12 | intron 2 + 2341 | aggtgccttctctattgtcc C/T acgcagacttaggtgatcct | 7266 | | |
| AADAC | 13 | intron 2 + 2546 | gtctgacacagaaggatcaa T/A ggcaaaatgtgcaagacaaa | 7267 | | |
| AADAC | 14 | intron 2 + 2609 | taggaggttcactgggaaac T/C tgaattccactgagtcatga | 7268 | | |
| AADAC | 15 | intron 2 + 2663 | tataaatacagtgttaaatt T/C gtctctcgtatcttaaggta | 7269 | | |
| AADAC | 16 | intron 4 + 605 | tgtgtcagtaaaatattata T/C taagtaggtgaatgagatca | 7270 | | |
| AADAC | 17 | intron 4 + 621 | tatattaagtaggtgaatga G/T atcatgtaattgtgagacta | 7271 | | |
| AADAC | 18 | intron 4 + 679 | ttagagattcagacgaattc A/G tctaatcttcgatggtgtat | 7272 | | |
| AADAC | 19 | intron 4 + 1680 | gttaaaatgtggataaatac C/T acaatttgcaaaatatttgg | 7273 | | |
| AADAC | 20 | intron 4 + 1748 | atttagaagttctatacatc T/C tttatagtatattacacact | 7274 | | |
| AADAC | 21 | intron 4 + 1771 | tatagtatattacacacttc G/A aaaacacaaaattatttttt | 7275 | | |
| AADAC | 22 | exon 5 + 238 | caagtcatctcttcaaattt A/G tcactgtggtactttgggga | 7276 | | |
| AADAC | 23 | exon 5 + 678 | ttagaaattggtctttctta A/G aatggtctagttaagttcca | 7277 | | |
| AADAC | 24 | 3′flanking + 208 | aatgctaaaaaaaaaaaaaa A/Δ cacagtgtgtttagtgaatt | 7278 | | |
| CES1 | 1 | 5′flanking − 983 | tatttccttagccagcggta T/C cacagtgtgtttagtgaatt | 7279 | | |
| CES1 | 2 | 5′flanking − 814 | tcacattgccttgacatcac A/C cctactgctcctccacccta | 7280 | | |
| CES1 | 3 | 5′flanking − 248 | agtcctgcaagggtgacacc G/Δ ttatgccacaagcagttggg | 7281 | | |
| CES1 | 4 | intron 1 + 22 | tgagtccttctgaagtcaaa T/Δ atgcggggcactttttgaaa | 7282 | | |
| CES1 | 5 | intron 1 + 30 | tctgaagtcaaatatgcggg G/T cactttttgaaatccttgtt | 7283 | | |
| CES1 | 6 | intron 1 + 1662 | aagggaatccctgacctgag C/A atgaccagcccagtggtttc | 7284 | | |
| CES1 | 7 | intron 1 + 1726 | cctccctgaagtcctcagca A/C tcttagctggttcctcgccc | 7285 | | |
| CES1 | 8 | intron 1 + 2716 | tgcttccaaggaagttcatc T/G cagtattatttgtaattagc | 7286 | | |
| CES1 | 9 | intron 1 + (2747-2749) | tgtaattagcaacaacaaca AAA/Δ gaaaagaagctaaatattga | 7287 | | |
| CES1 | 10 | intron 1 + 3288 | ttatttgtccattaaagaaa A/Δ ctcaagcgcttagcctggca | 7288 | | |
| CES1 | 11 | intron 1 + 3681 | gagaatatgggacacccctt T/C ttcatcctctcatccagcat | 7289 | | |
| CES1 | 12 | intron 1 + 3818 | tccttcttgcatttattttt A/G gctggatgtttttatgcctc | 7290 | | |
| CES1 | 13 | intron 1 + 3880 | aaccagctcaatgggttagg C/A aggacattgatcgtcatccc | 7291 | | |
| CES1 | 14 | intron 2 + 74 | gagtcaaggcagtcccctga T/C gggctgatcctttgctctgg | 7292 | | |
| CES1 | 15 | intron 2 + 552 | atggaaggtgtgtccattca C/A cctggccaagctgggaagaa | 7293 | | |
| CES1 | 16 | intron 2 + 885 | cagtattttagatggtaaag T/C attatgatgtaatatattgt | 7294 | | |
| CES1 | 17 | intron 2 + 2001 | ttggcatgtcagggctgcaa C/A actcatgtagaaatcactcc | 7295 | | |
| CES1 | 18 | intron 3 + 2119 | cgctgagtgcatgaatagtc T/C aggcttgagggtgatgggag | 7296 | | |
| CES1 | 19 | intron 4 + 127 | taaggcatccaagccccttc C/A taattggacactacctaccg | 7297 | | |
| CES1 | 20 | intron 4 + 347 | tctgtcatgacacttagcag T/C cagcccagcaggtgaaggtt | 7298 | | |
| CES1 | 21 | intron4 + (1984-1985) | gtggtcctgaaggtcctgca (C) tgacatctctgctccccacc | 7299 | | |
| CES1 | 21 | intron 4 + (1984-1985) | gtggtcctgaaggtcctgca tgacatctctgctccccacc | 7300 | | |
| CES1 | 22 | intron 5 + 766 | gaggtgggcagagggtcagc T/C cactactggattcctcagtc | 7301 | | |
| CES1 | 23 | intron 5 + 828 | ggagtagatctagcctggaa T/C agcgagtgagtcactgaccc | 7302 | | |
| CES1 | 24 | intron 5 + 828 | gtagatctagcctggaatag C/T gagtgagtcactgaccccac | 7303 | | |
| CES1 | 25 | intron 8 + 868 | ctcctgagcatgaactctcc T/A cccctccactctgctgtcag | 7304 | | |
| CES1 | 26 | intron 7 + 68 | acttcttcatttcagctgtc C/G tcttgcccagcgacggtttc | 7305 | | |
| CES1 | 27 | intron 7 + 681 | cctccaaaatcaacaatcca A/G ttatcgcctgtctgctagtt | 7306 | | |
| CES1 | 28 | intron 7 + 885 | aggaactatccaaagagaaa T/C acattcatatacttcgcagg | 7307 | | |
| CES1 | 29 | intron 7 + 2101 | gtcgtgtaaactgaaaatct C/G aggagttgatggcttcaggc | 7308 | | |
| CES1 | 30 | intron 7 + 2470 | atatagatatacgaattcac C/A gagtgatgcgggaacaacct | 7309 | | |
| CES1 | 31 | intron 8 + 128 | cgtgtttgtttctgaggccc A/G gagaggggtagtgactcacc | 7310 | | |
| CES1 | 32 | intron 0 + 2618 | cctgatggcaacacatgagt T/C gggctctctctaatctgtga | 7311 | | |
| CES1 | 33 | intron 0 + 2665 | aaaaattattcatcaaaggt C/A aaacctaaaattaagacatg | 7312 | | |
| CES1 | 34 | intron 8 + 3780 | ccatggcgcacggccatgcc C/A gtctatggtactggtctcac | 7313 | | |
| CES1 | 35 | intron 8 + 3791 | cgcatggccatgccggtcta T/C ggtactggtctcaccctcag | 7314 | | |
| CES1 | 36 | intron 10 + 222 | gtgggctggagaagctgcat C/T gctcaccaggggctggtggt | 7315 | | |
| CES1 | 37 | intron 10 + 230 | gagaagctgcatcgctcacc A/G ggggctggtggtcacttttt | 7316 | | |
| CES1 | 38 | intron 11 + 1177 | ctagcaggtgccctgacaca C/G ctttgcacaggaaggggcag | 7317 | | |
| CES1 | 39 | intron 11 + 1311 | gccctatgctctgcgtctga A/G ctatatatagacttcccatc | 7318 | | |
| CES1 | 40 | intron 11 + 2029 | ttctcatttgggatgctaag A/G ttaaaaattagcataacact | 7319 | | |
| CES1 | 41 | intron 11 + 2029 | catttgggatgctaagatta A/G aaattagcataacacttcca | 7320 | | |
| CES1 | 42 | intron 11 + 2317 | cattcacaaaagctctttct T/C ctatggttggctctgagttt | 7321 | | |
| CES1 | 43 | intron 11 + 3887 | caaatatttggctctaattc C/T gcttccacctcagacagctg | 7322 | | |
| CES1 | 44 | intron 12 + 2311 | gcgcctctgggcatctcact C/A tgcatgcttaggcgccttgc | 7323 | | |
| CES1 | 45 | intron 12 + 2331 | gtacatgcttaggcgccttg C/G ggctctgttgtttttcagaa | 7324 | | |
| CES1 | 46 | 3+ flanking + 71 | aacggtgatgaaagaggcga T/C gtgagaaggaaggtggcttt | 7325 | | |
| CES1 | 47 | 3′flanking + 362 | ttgcatggcacttactgacc C/A ttgcacaggcctgcaacacc | 7326 | | |
| CES1 | 48 | 3′flanking + 581 | atttctggattctgttagta C/T gtagaaagctctaaagcatg | 7327 | | |
| CES1 | 49 | 3′flanking + 1348 | aaatctgctgctgggagaga G/C agcaaagcatgcagatcaac | 7328 | | |
| CES2 | 1 | intron 1 + (1303-1321) | caacagtgacttgtctctacc (A)17-19 gtcaagcatggtggcagaca | 7329 | | |
| CES2 | 2 | exon 5 + 60 | ggacccagtggctccactac C/A ctgggtccagcagaatatcg | 7330 | | |
| CES2 | 3 | exon 12 + 256 | agcctgctgtgcccacacac A/G cccactaaggagaaagaagt | 7331 | | |
| CES2 | 4 | 3′flanking + (155-172) | acacagtgagaccccttctc (A)16-18 gagagagtgtgtgattagaa | 7332 | | |
| CES2 | 5 | 3′flanking + (173-178) | tcaaaaaaaaaaaaaaaaaa (GA)4-6 gtgtgtgattagaagctaaa | 7333 | | |
| CES2 | 6 | 3′flanking + 377 | ggtcaaggtgagcagaacac C/G tgaggacaggagtttgagac | 7334 | | |
| GZMA | 1 | 5′flanking − 424 | cctcagcttgcacttggcct A/G ctaattcttatataacccaa | 7335 | | |
| GZMA | 2 | 5′flanking − 134 | agcctgcctgctggcagtga C/G ccatcatccaccattctgac | 7336 | | |
| GZMA | 3 | intron 1 + 1947 | gacataaggttctctctatc A/T gcatgtatggtttgccttgt | 7337 | | |
| GZMA | 4 | intron 2 + 958 | gactgcgtgaccaggtagaa C/T tagcctcagcatggaagggt | 7338 | | |
| GZMA | 5 | intron 2 + 1025 | gttggtgtagtttatactag C/A ttatgaatgatagccttaat | 7339 | | |
| GZMA | 6 | intron 4 + 105 | tgccaagttgcagggtgggg C/G aggactcacaatagtgcatc | 7340 | | |
| GZMA | 7 | intron 4 + 696 | atagagccttacctgaagaa A/G ggtgtgcagtatgcatggtt | 7341 | | |
| GZMA | 8 | intron 4 + 1141 | ctgttcagggaggatcccgg C/A ttccaacatggttctttatt | 7342 | | |
| GZMB | 1 | 5′flanking − 961 | tgtttcgcaaatgtttactg T/C gagcctgttatgtgctgagc | 7343 | | |
| GZMB | 2 | 5′flanking − 263 | ggctgataccacatcctaca A/G ttcacttcataggcttgggt | 7344 | | |
| GZMB | 3 | exon 2 + 109 | gtgcggtggcttcctgatac A/G agacgacttcgtgctgacag | 7345 | | |
| GZMB | 4 | intron 2 + (242-243) | cgcgggcatactttggcata (A) gaatacaaactgaagcaatt | 7346 | | |
| GZMB | 4 | intron 2 + (242-243) | cgcgggcatactttggcata gaatacaaactgaagcaatt | 7347 | | |
| GZMB | 5 | intron 4 + 131 | atttctctctggaaagagaa G/A aggggactagactgagctgg | 7348 | | |
| GZMB | 6 | intron 4 + 102 | gggcctctgcaaacttacca C/A gaggcttatggtggatggtg | 7349 | | |
| GZMB | 7 | 3′flanking + 54 | attctcaggcaccacatctg C/T gctatgcaggccaatgacac | 7350 | | |
| GZMB | 8 | 3′flanking + 184 | tccacaccagtttctccagg C/T cctgcccttctgccaaggct | 7351 | | |
| GZMB | 9 | 3′flanking + 286 | ccactttggtcctggggctt T/A gggtaaacttcttacctcct | 7352 | | |
| GZMB | 10 | 3′flanking + 406 | ctgagctcaaggctcagctc C/A tcctccagccctttggctgc | 7353 | | |
| ESD | 1 | 5′flanking − 333 | gtcttgggacagaggagttg C/A gggagttgaaattaggccct | 7354 | | |
| ESD | 2 | intron 1 + 603 | gtcatttctgatggggtcat C/T agggaaatgggattgagcgc | 7355 | | |
| ESD | 3 | intron 1 + 698 | tgtgtggtagaagaagcatt C/T taagcactacgtgaattaac | 7356 | | |
| ESD | 4 | intron 1 + 1864 | gctttcatgcaggattgatc C/G tagtgggatgtattaggaag | 7357 | | |
| ESD | 5 | intron 1 + 2389 | ttttgggaacacctgtctag G/A ttgttaagagccagtggaat | 7358 | | |
| ESD | 6 | intron 2 + 22 | taaacttgttttattgttta T/C atgttactctgaacattgaa | 7359 | | |
| ESD | 7 | intron 2 + 589 | taaaattagtatctctctct G/A taagttcattatttaagata | 7360 | | |
| ESD | 8 | intron 2 + 1499 | tagaaaaatgtgtatcacac C/T gtaagtgttcagtaatgtta | 7361 | | |
| ESD | 9 | intron 3 + 92 | ctttatctagatattatagt C/A cctcattttacttttaaact | 7362 | | |
| ESD | 10 | intron 3 + 422 | gtaaagagattaaacacaca C/T gcacacatacatatacctat | 7363 | | |
| END | 11 | intron 3 + 581 | agaaaacctgagaaatgaea C/T aatttatttaaagccatagt | 7364 | | |
| ESD | 12 | intron 3 + 2270 | gccagtaattacatgtagcc C/A tttacatcaaattagctaat | 7365 | | |
| ESD | 13 | intron 3 + 2951 | taatgaaagtaaatgtttca A/G cttccctaacaaaagttgaa | 7366 | | |
| ESD | 14 | intron 3 + 3503 | aaatgtcagaaattttttgt C/A ccgtcagtcatcaacaagaa | 7367 | | |
| ESD | 15 | intron 3 + 3097 | aaggagcatacagaaaactt C/G ccatgatggggcctttgtgg | 7368 | | |
| ESD | 16 | intron 4 + 2616 | tctaatagtccccagtatta A/G tttacatcaaattagctaat | 7369 | | |
| ESD | 17 | intron 5 + 392 | tcttttttcatctctgttaa C/T atcaaccatacagttaaaca | 7370 | | |
| ESD | 18 | intron 7 + 107 | ttagtattggaactaaactt T/C tctagtgttgagaactttgg | 7371 | | |
| ESD | 19 | intron 8 + 1091 | aaattctaactaattaaagg G/T ttcatcctttagtaactaga | 7372 | | |
| ESD | 20 | intron 8 + 1652 | tataaacttgtggttaatga A/G tatatatgaataagaatatt | 7373 | | |
| ESD | 21 | intron 8 + 2048 | agaaggaaaaaggccatttt G/C ttaagaatccctgagatatg | 7374 | | |
| ESD | 22 | intron 9 + (1523-1526) | ctgccaacaaagtctgaaaa (TC)2-3 aagtttgttataaaaacagc | 7375 | | |
| ESD | 23 | intron 9 + 3468 | atagaaggagaggctatact A/G cctccttaagtctcaggacc | 7376 | | |
| ESD | 24 | intron 9 + 3362 | actaaggataaaaatatggc A/G tactcagtcacattggaact | 7377 | | |
| ESD | 25 | intron 9 + 5292 | aggccttaatgacatatttc T/C cctcacataaagatacaaca | 7378 | | |
| ESD | 26 | intron 9 + 5298 | taatgacatatttcccctca A/G ataaagatacaacatgcttt | 7379 | | |
| ESD | 27 | 3′flanking + 798 | tatggtaactgaagaaaatg A/G cattaagttcctaaagttat | 7380 | | |
| CEL | 1 | 5′flanking − (611-617) | tggatcaaggcaaataattt (A)6-7 ggaaattatttgaagaaaaa | 7381 | | |
| CEL | 2 | intron 1 + 20098 | atctctaccaaggtaccaat T/C ccttaaggaagatgttaatt | 7382 | | |
| CEL | 3 | intron 1 + (20911-20924) | taatgacattaatacttagc (A)13-15 ctgaatatgactaaaactga | 7383 | | |
| CEL | 4 | intron 1 + 22374 | ttaagttaaatgtaaacagc A/G cctttgcacactattcagtg | 7384 | | |
| CEL | 5 | intron 1 + (22460-22469) | ttaattttttagttaggttg (T)9-10 ctcttttattttatcacatg | 7385 | | |
| CEL | 6 | intron 1 + 24205 | agaatttgagtctattcttg T/C gtgccttctgactacatcct | 7386 | | |
| CEL | 7 | intron 1 + (24404-24417) | gcaagttttaactgaattac (A)11-14 gcagatgataatcattctat | 7387 | | |
| CEL | 8 | intron 1 + 26983 | tagattttgatgagtttgag T/G ttttttttttttttttccaa | 7388 | | |
| CEL | 9 | intron 1 + (26983-26999) | tagattttgatgagtttgag (T)14-17 ccaaaagggtgggggttgtt | 7389 | | |
| CEL | 10 | intron 1 + (32166-32174) | tcaactttgctggtaaccag (A)8-9 gaaaagccactattaatatc | 7390 | | |
| CEL | 11 | intron 1 + 37217 | aaatttgtaagtgaatgtta T/C ataaaaatctgtaacaatta | 7391 | | |
| CEL | 12 | intron 1 + 37685 | taattcaaatggattaatca T/A tgataatttctatttttaaa | 7392 | | |
| CEL | 13 | intron 1 + 38032 | caggcctaataaatgaaatg T/C tcactactgttgccaacacc | 7393 | | |
| CEL | 14 | intron 1 + 38133 | attcgggagtcctgtctgcc A/G tttgtagaaaccatccagct | 7394 | | |
| CEL | 15 | intron 1 + 38169 | cagctcatcttcctactctt A/T gtgttggggatttttgcccc | 7395 | | |
| CEL | 16 | intron 1 + 38544 | gtttctgtcaactctccaga T/C ataaaatcaaatgctcttcc | 7396 | | |
| CEL | 17 | intron 1 + (38642-38643) | caatttcttcacaatacctg (G) attgctgccaggcagcaata | 7397 | | |
| CEL | 17 | intron 1 + (38642-38643) | caatttcttcacaatacctg attgctgccaggcagcaata | 7398 | | |
| CEL | 18 | intron 1 + 49429 | gaaagagaaacttgtgtccc A/G gaaacttgtgtaagtatgcc | 7399 | | |
| CEL | 19 | intron 1 + 49038 | ttgaaactgcactgacacta A/G tttaaattttacaagtaatt | 7400 | | |
| CEL | 20 | intron 1 + 49040 | gaaactgcactgacactaat T/C taaattttacaagtaatttt | 7401 | | |
| CEL | 21 | intron 1 + 49256 | acatgagaaaagaaatggag C/A taagtttaaaaacagaatga | 7402 | | |
| CEL | 22 | intron 1 + 49386 | aatagttctcagtagatatt C/A ttttacctatatttagtata | 7403 | | |
| CEL | 23 | intron 1 + 50786 | tactttgtcctcaccaatgc C/A tattcttcccctaaacagat | 7404 | | |
| CEL | 24 | intron 1 + 50977 | ctccagccagagaggacaga T/C agctgagtttctgtttgcct | 7405 | | |
| CEL | 25 | intron 1 + 51150 | agcacnatggactgtttttg C/G agtcctcctctttattatgc | 7406 | | |
| CEL | 26 | intron 1 + 52333 | tcagtcaaacttaaaggctc A/G gagatctattaatgcttatg | 7407 | | |
| CEL | 27 | intron 1 + 52589 | gtgtcagcatctgtagagta C/A gggagggtgttgaaagaaaa | 7408 | | |
| CEL | 28 | intron 1 + 55838 | tctcgcaggtaaatgaggat C/A gaatactttaaatacaaatc | 7409 | | |
| CEL | 29 | intron 1 + 56828 | ataagtttggaaaatttgtg C/G taaaatacactaaatatttc | 7410 | | |
| CEL | 30 | intron 1 + 58738 | tggtggagaaatagcttata C/A tgctggtcaaactgtcccat | 7411 | | |
| CEL | 31 | intron 1 + 59358 | cagaaattgtactttaaaat A/G cgaactgcaagcactgcagt | 7412 | | |
| CEL | 32 | intron 1 + 59359 | agaaattgtactttaaaata C/T gaactgcaagcagtgcagtc | 7413 | | |
| CEL | 33 | intron 1 + 59464 | acccagaaaggagcatgtcc C/G tttgtcatttgtggtgaaac | 7414 | | |
| CEL | 34 | intron 1 + 61340 | aaaaaaaacttcaaaatact C/G caatatccaaagttggtaca | 7415 | | |
| CEL | 35 | intron 1 + 62739 | cagtctttaggcacaaagag A/G caaagagtcttctcatctct | 7416 | | |
| CEL | 36 | intron 1 + (64764-64779) | ctgggttgtttctcataaag (T)14-17 aatgtgggatagtggtataa | 7417 | | |
| CEL | 37 | intron 1 + 65243 | tttcaggcttctggacagaa T/C agtattatgataaaagctat | 7418 | | |
| CEL | 36 | intron 1 + 65269 | tatgataaaagctattaata T/A ttaggaagattcctctgact | 7419 | | |
| CEL | 39 | intron 1 + 65325 | aattagaaaagcaagttttg C/G gggggggggtccaaaacaaa | 7420 | | |
| CEL | 40 | intron 1 + (65326-65334) | attagaaaagcaagttttgg (G)7-9 gggggggggtgcaaaacaaa | 7421 | | |
| CEL | 41 | intron 1 + 65524 | cacacccataaccaccagtt A/G gttgcctctcctgagccatg | 7422 | | |
| CEL | 42 | intron 1 + 65869 | cagagtaacattcgggctcc A/T actgtcctttcttatacaga | 7423 | | |
| CEL | 43 | intron 1 + 65910 | aaggctgtctcctgctgttt C/G tggatccaaggcctgctgaa | 7424 | | |
| CEL | 44 | intron 1 + 66800 | cctctctttccatccctgac C/A gagcatattcactgtcctat | 7425 | | |
| CEL | 45 | intron 1 + (66226-66235) | tctgtttttgaaaaaacaag (A)9-10 tctctccctgcctttggaaa | 7426 | | |
| CEL | 46 | intron 1 + 81816 | aatgttgccttactttccac A/G tatttccagaagccctgatc | 7427 | | |
| CEL | 47 | intron 1 + 83480 | tatgactgtcaggaagaaaa T/C tagaattatctttgtatcct | 7428 | | |
| CEL | 48 | intron 1 + 83732 | ggggtttgaaatctatggag T/C catttacttctttttaaaaa | 7429 | | |
| CEL | 49 | intron 1 + 85507 | ctggaaagaaattttgtgta A/T ctgcattatttaaatgttag | 7430 | | |
| CEL | 50 | intron 1 + 87299 | caatggtcattatatcttcc G/A tgtgtgaagaaagtcgacaa | 7431 | | |
| CEL | 51 | intron 1 + 87426 | caacaggataatcccagaat G/C ctctgtctgcacttggctct | 7432 | | |
| CEL | 52 | intron 1 + 87670 | tattttgttcctaatattca T/C gacatgacacaccaacataa | 7433 | | |
| CEL | 53 | intron 1 − (77484-77503) | ttggttactgttttttattt (A)9-10 aaactctgataaaaggggcc | 7434 | | |
| CEL | 54 | intron 1 + 77368 | agctcacgggacagaacact G/C gggggaggcaagaaagcggg | 7435 | | |
| CEL | 55 | intron I − (75135-70129) | tgcccggctggcacaagccct (G)6-7 tgggactactctgacgcctc | 7436 | | |
| CEL | 58 | intron 1 − 74785 | gctgcccacggaagctgggg G/C ctgttcgcctcttctcctgt | 7437 | | |
| CEL | 57 | intron 1 − 74755 | tcttctcctgtgtacatgaa A/G cctcaggcctccaggtgcag | 7438 | | |
| CEL | 58 | intron 1 − 73099 | ccccgcggtctactcctggc C/T tcttcttgccgccgcctgct | 7439 | | |
| CEL | 59 | intron 1 − 72559 | agcagcagctgggccggtcc G/A tgcagggagtgaggtcggca | 7440 | | |
| CEL | 60 | intron 1 − 70098 | acagggaggaaacagcaaaa T/C ctcaacactgttgatctaat | 7441 | | |
| CEL | 61 | intron 1 − 69440 | gttggccatgagagaaaaca C/T aggaaggtattggaaaatga | 7442 | | |
| CEL | 62 | intron 1 − 65270 | attctgcactggctgggaag G/Δ cttggcttgggcttcatggc | 7443 | | |
| CEL | 63 | intron 1 − 64434 | ccaaattagggagtggaatg C/T aaaatctgaattaattttca | 7444 | | |
| CEL | 64 | intron 1 − 63966 | agatcacacatcccccaccc C/T atcgcctagagaactgagcc | 7445 | | |
| CEL | 65 | intron 1 − 63916 | gctgctcaccatgacctagc C/T ttcagggctgaccccagtcc | 7446 | | |
| CEL | 66 | intron 1 − 60392 | tcatggggctccaggatgca C/T gtggaaatccctgggagaag | 7447 | | |
| CEL | 67 | intron 1 − 60321 | aattacttgaaccccattcc A/T tcccacccaaacccttttcc | 7448 | | |
| CEL | 68 | intron 1 − 60318 | tacttgaacaaaattcaatc C/T cacacaaacaattttcctcc | 7449 | | |
| CEL | 69 | intron 1 − 56852 | tgctccaagccctccccctg C/A gcccagcacgaccccatctc | 7450 | | |
| CEL | 70 | intron 1 − 56133 | gctggctcgtgggatgtcta C/T ggggattgactggcaccccc | 7451 | | |
| CEL | 71 | intron 1 − 55964 | ccccagcgcccatcagaccg a/A cctgagacttatcactgccc | 7452 | | |
| CEL | 72 | intron 1 − 52016 | tactggaactaggggtgggg G/A ggcactgccagtggaaaggg | 7453 | | |
| CEL | 73 | intron 1 − 51998 | gggggcactgccagtgccca G/A gggaggggactgcggggcac | 7454 | | |
| CEL | 74 | intron 1 − 51578 | gtgggatcgacttgcatttt T/C gggggagaagaataactggt | 7455 | | |
| CEL | 75 | intron 1 − 39557 | ggaccagcacatggattcaa T/C gaggctctaagctacccaag | 7456 | | |
| CEL | 76 | intron 1 − 39490 | gccctttcttccaggttgta A/G tgggcaatgatggtcaccag | 7457 | | |
| CEL | 77 | intron 1 − (31332-31340) | tccggacttctaattggctc (A)8-9 ctcgctcggccctcggattc | 7458 | | |
| CEL | 78 | intron 1 − 19634 | ttatttaagggctggaaata C/T tagctgcctgcaggagatgt | 7459 | | |
| CEL | 79 | intron 1 − 6589 | gacgggtgatgcgagggact T/C gctgtccccaagtgtatggg | 7460 | | |
| CEL | 80 | intron 1 − (3340-3348) | gctggcagtgctggcctgtc (C)4-6 tcacatgtggtcgggttggg | 7461 | | |
| CEL | 81 | intron 3 + 35 | tgcaggaatggccatgagga G/A gggcgggtgagggcggctga | 7462 | | |
| CEL | 82 | intron 6 + 157 | gtggggagcggccttggtga C/T gggatttctgggtcaagtag | 7463 | | |
| CEL | 83 | exon 9 + 137 | aacatggacggacacatctt C/T gcaagcatcgacatgcctcc | 7464 | | |
| CEL | 84 | intron 9 + 41 | tcaggggcgacacgtgcggg A/G gggcagccgggaaagcactg | 7465 | | |
| CEL | 85 | intron 9 + 181 | ggggtgagtatgcacacaca T/C tcctgttggaaaaggatgag | 7466 | | |
| CEL | 86 | exon 10 + 82 | acgacctttgatgtctacac C/T gagtcatgggaccaggaccc | 7467 | | |
| CEL | 87 | exon 12 + 583 | cccacgggtgaatacggggc C/A cacccagtgaacaacaaggg | 7468 | | |
| CEL | 88 | exon 12 + 759 | gttttagagtaacatgagca T/C tggtatcaagaggccacaag | 7469 | | |
| TL17 | 1 | 5′flanking + 832 | cctgagaaggaactattctc A/G aggaaatgagtaaaacttaa | 7470 | | |
| TL17 | 2 | 5′flanking + 692 | tgccccccttttatcaatct C/T cataacctttctccagtctc | 7471 | | |
| TL17 | 3 | 5′flanking + 76 | ccctgaacccactgcgacac G/A ccacgtaagtgaccacagaa | 7472 | | |
| TL17 | 4 | intron 1 + 18 | gtggtgagtcctgcactaac G/A tgcgatcctcttgatgattt | 7473 | | |
| TL17 | 5 | intron 1 + 126 | ctgtatatgtaggataggaa A/G tgaaagctttggtaggtatt | 7474 | | |
| TL17 | 6 | intron 1 + 762 | ctgagaacaatggtgcagga G/A gatatttataaatagaaaat | 7475 | | |
| TL17 | 7 | intron 2 + 594 | tattttgatcatttgacttc A/T tacaaataagtctctgttct | 7476 | | |
| TL17 | 8 | exon 3 + 1487 | agctgatggggaagaacgaa C/T tttaagtatgagaaaactta | 7477 | | |
| TL17 | 9 | 3′flanking + 657 | ccctgaatctttttaattat G/T catctaactaattcctaaca | 7478 | | |
| UCHL3 | 1 | 5′flanking − 1034 | ataatgtgaagaagaaaaaa A/G agaaactgctactgggctac | 7479 | | |
| UCHL3 | 2 | 5′flanking − 490 | aactactgcacaccgacaaa G/C gaacaacagcaacgtgctga | 7480 | | |
| UCHL3 | 3 | 5′flanking − 400 | aaaagaaaaacgaaaaacag T/C accgtgatgaacggcgtcct | 7481 | | |
| UCHL3 | 4 | 5′flanking − 295 | atgcgtagaacgcgagcgct T/C ggaaaggctaagctcggaag | 7482 | | |
| UCHL3 | 5 | 5′flanking − (25-11) | tgggcggaagcggcggcggc GGCGAAGGCGGCGGC/Δ tgtcagagctg | 7483 | | |
| UCHL3 | 6 | intron 2 + 28 | aggtgtctgtcgctcgggac T/C tcggagtcttttctgtctgc | 7484 | | |
| UCHL3 | 7 | intron 2 + (5639-9640) | aattttttattataataata (ATA) tataagtagaagaattatat | 7485 | | |
| UCHL3 | 7 | intron 2 + (5639-5840) | aattttttattataataata tataagtagaagaattatat | 7486 | | |
| UCHL3 | 8 | intron 2 + 7062 | aggtggattcaaaccaaaca G/A gctaactgctaacattttag | 7487 | | |
| UCHL3 | 9 | intron 2 + (7936-7947) | tgatcatttcaaacacaggg (T)11-12 aattgtaaaagtaggacatt | 7488 | | |
| UCHL3 | 10 | intron 2 + (7975-7988) | aaagtaggacattttaatga (T)12-14 gaagaagtgagtggtaaaag | 7489 | | |
| UCHL3 | 11 | intron 2 + 8117 | cctgactcatggcaatatgg A/G gtaaggatctaacaatatat | 7490 | | |
| UCHL3 | 12 | intron 2 + 8361 | ttcttacctttacctgacat G/A gagtagatttgcagtgaact | 7491 | | |
| UCHL3 | 13 | intron 2 + 9800 | taagatatagtgatgcattt C/T taatatgatttttgtttcct | 7492 | | |
| UCHL3 | 14 | intron 2 + (10735-10747) | taccaactaatgttccattg (T)9-10 ctttctttcttttaccagtt | 7493 | | |
| UCHL3 | 15 | intron 3 + 11 | tacagaaaaggtaattgtta A/T gtaaaatagaaagtttctgg | 7494 | | |
| UCHL3 | 16 | intron 3 + (662-675) | cttaaatacaattttttcaa (TA)6-7 aggaatattctttgattatt | 7495 | | |
| UCHL3 | 17 | intron 3+ 866 | tcaagtctacatattttagt T/C tttttttctagaatgatata | 7496 | | |
| | | | tacatacgtatacgtatata | | | |
| UCHL3 | 18 | intron 3 + (94 + 945) | (TGTATACGTATACATACGTATACATATATACATACGTATATA) | 7497 | | |
| | | | cgtacgtatatacgtatacg | | | |
| UCHL3 | 18 | intron 3 + (94 + 945) | tacatacgtatacgtatata | 7498 | | |
| | | | cgtacgtatatacgtatacg | | | |
| UCHL3 | 19 | intron 3 + 5052 | aggcagtcagctatagagcc T/C acatttttgatgcttattat | 7499 | | |
| UCHL3 | 20 | intron 3 + 5282 | acctctgttaagtttttgca T/C accctttcagactttccaat | 7500 | | |
| UCHL3 | 21 | intron 6 + 2191 | tttctaggggtttcctagtg C/T gtagagcagtgattctcaag | 7501 | | |
| UCHL3 | 22 | intron 6 + 8284 | tctgcaagtcaaatgtgaag C/G caagagagaaaaatccaaaa | 7502 | | |
| UCHL3 | 23 | intron 6 + (8741-8744) | atgtgagtaaaccacaattt ATTT/Δ ttcatttccttaacttttga | 7503 | | |
| UCHL3 | 24 | intron 6 + 9411 | tcctctgtttagaatctact T/G ggcttttttggcccagccag | 7504 | | |
| UCHL3 | 25 | intron 6 + 9459 | tgtcagtggcagtaaatagt T/A taaagtttcatcttcattac | 7505 | | |
| UCHL3 | 26 | intron 6 + 9772 | gaaacaatacatgtatcatg T/C ggttcaagatgtagagtcca | 7506 | | |
| UCHL3 | 27 | intron 6 + 10158 | ttattttaaaggaaaattct C/T agaccgaacttaccagttca | 7507 | | |
| UCHL3 | 28 | intron 6 + 10829 | tttactaaaaaatctacaga A/C atccatttagaattaattta | 7508 | | |
| UCHL3 | 29 | intron 6 + 12493 | agtcaaattagttgacagtt A/G atgggcgagtgaccttgcaa | 7509 | | |
| UCHL3 | 30 | intron 6 + (20435-20437) | ttttttaattatgtagtcct CCT/Δ cgccatcctcatcacagcct | 7510 | | |
| UCHL3 | 31 | intron 6 + 21202 | ttgatctgatctttcctgcc C/T attcagtttctaaagatctt | 7511 | | |
| UCHL3 | 32 | intron 6 + 21295 | caaatttatgatttctcttt T/C ataggctaatgatatctgca | 7512 | | |
| UCHL3 | 33 | intron 6 + 21639 | taagaacaattaaaagtcaa C/T ggcaagcattctttccttcc | 7513 | | |
| UCHL3 | 34 | intron 6 + 21778 | tccatttctgctgagtatca A/G caaactcacatctctttcta | 7514 | | |
| UCHL3 | 35 | intron 6 + 23299 | cttttagattaaaggtgcaa T/C gatgcacaattttgagtcac | 7515 | | |
| UCHL3 | 36 | intron 6 + 23498 | tattcagttctctgactcca A/G ttgtactacttttacctcta | 7516 | | |
| UCHL3 | 37 | intron 6 + 23790 | ctagccttaaaaaattggac A/T ctcttctgattattgataaa | 7517 | | |
| UCHL3 | 38 | intron 6 + 23894 | actcattatcactgtcttca A/G atatttaaagaaatatgttc | 7518 | | |
| UCHL3 | 39 | intron 6 + (24729-24732) | agtcttaatttcaaattgtt TGTT/Δ aagcatcaaagcaagagaaa | 7519 | | |
| UCHL3 | 40 | intron 6 + (29083-25084) | catgtattcatttcattcag (A) taagtatgcaatgtccatat | 7520 | | |
| UCHL3 | 40 | intron 6 + (25083-25064) | catgtattcatttcattcag taagtatgcaatgtgcatat | 7521 | | |
| UCHL3 | 41 | intron 6 + 25884 | catgtattcatttcattcag C/T aagtatgcaatgtgcatata | 7522 | | |
| UCHL3 | 42 | intron 7 + 1342 | gaagaagtcattattttggt C/A gcatataatggacctccagg | 7523 | | |
| UCHL3 | 43 | intron 7 + 1387 | ttttgaagatgtgccttgct G/A attgagtctacaaaatctgc | 7524 | | |
| UCHL3 | 44 | intron 7 + 1760 | actcggttttactagttaga T/C agctgtcttggctcagaggc | 7525 | | |
| UCHL3 | 45 | intron 7 + 2096 | taggtacattacaaagatgg C/A cagttgctgattcgttgcaa | 7526 | | |
| UCHL3 | 46 | intron 7 + 2873 | ttaatgtattaattccctac T/C ctaataaattgtaaggttaa | 7527 | | |
| UCHL3 | 47 | intron 7 + 7594 | tctctgagcctcatggattc T/A tctgcagcgtatgcatttac | 7528 | | |
| UCHL3 | 48 | intron 8 + 287 | ctctatgaacaaatgtaaaa T/A ttgaaaaggcaagaatagta | 7529 | | |
| UCHL3 | 49 | intron 8 + 252 | aagacttgctcattatatcc C/G agatttcatnaaatccagga | 7530 | | |
| UCHL3 | 50 | intron 9 + (553-592) | tttacactgaaaaatcatac (T)9-10 ccggcatagcatgccataca | 7531 | | |
| DDOST | 1 | intron 2 629 | attctgttaagaagttctta T/C atgaacaaatattgtctcct | 7532 | | |
| DDOST | 2 | intron 2 3125 | gagaatataggagcttctgc C/A tatgcctgaaagtcagtcag | 7533 | | |
| DDOST | 3 | intron 2 3920 | attactcatttgatgaataa A/G tggattactgagcactgtct | 7534 | | |
| DDOST | 4 | intron 3 189 | actgctgtccaggggtccat C/T tggggctgagcccacctgga | 7535 | | |
| DDOST | 5 | intron 6 185 | ctgtcctcttgttcgggagg C/T gtggcagcttttcccctact | 7536 | | |
| DDOST | 6 | exon8 37 | aactatgaactagctctggc C/T ctctcccgctgggtgttcaa | 7537 | | |
| DDOST | 7 | intron9 37 | tcctgcccaagaatgctgcc A/Δ aaaaacggccccacgcctca | 7538 | | |
| DDOST | 8 | intron2 + 1299 | atcttctgatggctgggctt C/T ggtgcagtaactggtgtttg | 7539 | | |
| DDOST | 9 | intron2 + 1581 | gatactgtgggtgggagaaa T/C gacagagagtgtaaaacagt | 7540 | | |
| DDOST | 10 | intron2 + 2822 | gtttctcaacaggtgcattc T/C tgacgtttcagactggatac | 7541 | | |
| DDOST | 11 | intron2 + 3392 | cagaaggcgtggaggcctgc C/T gcgcctccctctgttgctgc | 7542 | | |
| DDOST | 12 | intron5 + 495 | attgcttgaacccaggacgc C/A gaggttgcagtgagccaagg | 7543 | | |
| DDOST | 13 | intron6 + 226 | ggaactgcttgggtcacagc C/T ccgccttgcccccagtaccc | 7544 | | |
| DDOST | 14 | intron6 + 303 | aagagagataggtcattagg A/T tgaatttgttaggcaagaga | 7545 | | |
| DDOST | 15 | 3′flanking + 40 | cacagcgtggagacggggca C/A ggaggggggttattaggatt | 7546 | | |
| NTE | 1 | 5′flanking − 535 | cacgatctgtcctccgattc C/T tgttaactctagactttctg | 7547 | | |
| NTE | 2 | 5′flanking − 15 | gtaaatccccggcaaaaacc A/G gcagcgccttgcaagcccac | 7548 | | |
| NTE | 3 | 5′flanking − 748 | agcatggcgcggggaggagg C/T gtgggagggtcgggagggac | 7549 | | |
| NTE | 4 | 5′flanking − 690 | tgaataatttaaaggggccg T/C gccggcggagccgggcggaa | 7550 | | |
| NTE | 5 | intron6 + 605 | ccttgccatatacttagtgg A/G ggggtctacatcaggggttt | 7551 | | |
| NTE | 6 | intron6 + 749 | agcctccagcctctcttctc C/T gggggttatctcaggcatct | 7552 | | |
| NTE | 7 | intron6 + 957 | ggtctgggctctgggatccc C/T gtgcgtcatgtagtctacct | 7553 | | |
| NTE | 8 | intron6 + 1882 | tggcctcaagcaatcctccc C/A cctcggcctcccaaagcgcc | 7554 | | |
| NTE | 9 | intron6 + 2222 | gaatgtttatgtagaacaga C/A agactgtatctgcggtcttc | 7555 | | |
| NTE | 10 | intron12 + 166 | tatctggtaccgaggaagct C/G cggcctcgtccccaagggcc | 7556 | | |
| NTE | 11 | intron13 + 69 | atccaggtccaccgcctgcc C/T gtcttgattgttttaatctg | 7557 | | |
| NTE | 12 | intron14 + 8 | agcccccgctcgggtaaggc C/T tgggaccctgcccgctggtg | 7558 | | |
| NTE | 13 | intron16 − 113 | cccaccccgcccggcgccgt T/C atatttttcttaacccttcc | 7559 | | |
| NTE | 14 | intron21 + 34 | agagcccgccggcccagagc A/G tgctgggagatgtagtccgg | 7560 | | |
| NTE | 15 | intron21 + 128 | gaagaaatcgtgccgctgag C/A gtttcaaaccctaagtagga | 7561 | | |
| NTE | 16 | intron21 + 151 | ctcaaaccctaagtaggacc C/G aggtgcagagcattctgggc | 7562 | | |
| NTE | 17 | intron21 + 601 | ccactgtactcaagccggga C/T gacagagctagaacctcttt | 7563 | | |
| NTE | 18 | intron21 + 737 | tggaaaatagtctgtggatt G/T ttgtttaggactctgggcac | 7564 | | |
| NTE | 19 | intron21 + 1752 | acagctggtctaggctgtta G/C tggagaaactgggaagcaac | 7565 | | |
| NTE | 25 | intron31 + 1785 | gaagcaacagctgggtcaaa A/Δ gtagcttttcttttcttggc | 7566 | | |
| NTE | 21 | intron21 + 1907 | cactgcaacctctgcctccc A/G ggttcaagtgattctcctgc | 7567 | | |
| NTE | 22 | intron21 + 2065 | ctgcctcgttttatgttcag G/T tcccccattagacagaggaa | 7568 | | |
| NTE | 23 | intron21 + 2336 | agtctgggagcacaggagca G/A gaatttcagataaggaggaa | 7569 | | |
| NTE | 24 | intron23 + 41 | tggggagggtggtgggtggg G/C ctggagcctcaaattctttc | 7570 | | |
| NTE | 25 | intron23 + 71 | caaattctttcagacctgag T/C tcaagttctcggcttccaac | 7571 | | |
| NTE | 26 | intron23 + 81 | cagacctgagttcaagttct C/T ggcttccaaccacggagcct | 7572 | | |
| NTE | 27 | intron24 + 150 | gtggggcggctggtgacctc A/G gccgtccgtattccgcagct | 7573 | | |
| NTE | 28 | intron29 + 37 | gcctgcagcaaccgctgacg T/C cacgtggggttggggggatg | 7574 | | |
| NTE | 29 | intron29 + 370 | cgtcccaggtcagcgagccc G/A tcgggccggctgggcctccg | 7575 | | |
| NTE | 30 | intron30 + 56 | acctcccgcaccacacacac G/A cacacgcgtgggcacacaca | 7576 | | |
| NTE | 31 | intron30 + 358 | aaaaatacaaaaaattaacc A/G ggctggtggggtgtgcctgt | 7577 | | |
| NTE | 32 | intron30 + 372 | ttaaccaggctggtggggtg T/C ctggagcctcaaattctttc | 7578 | | |
| NTE | 33 | intron30 + 430 | aaatcacttgaacctgagag G/T tggaggttgcagtgagctga | 7579 | | |
| NTE | 34 | intron30 + 655 | gtgtgcacaccagctatata T/C gcaaatgctttctctcaggg | 7580 | | |
| NTE | 35 | intron30 + 655 | gcacaccagctatatataca A/G atgctttctctcaggggcag | 7581 | | |
| NTE | 36 | intron30 + 760 | tgaaatagggcatttgccaa C/T gcatgccagtctgtcccgtt | 7582 | | |
| NTE | 37 | intron30 + 835 | gcacacacgtagataggatg T/C ggcacctctgaccgagttaa | 7583 | | |
| NTE | 38 | intron31 + 40 | tggtgcctgcataggtggtc T/C ggctaagctttgctacttaa | 7584 | | |
| NTE | 30 | intron31 + 41 | ggtgcctgcataggtggtct G/A gctaagctttgctacttaaa | 7585 | | |
| NTE | 40 | intron31 + 1329 | gtctgtcaagggcaggacag G/A aaatatgtaaacgaatgtac | 7586 | | |
| NTE | 41 | intron35 + 31 | aatggcttcctgtcgttttc G/A gactggggacccaccttctg | 7587 | | |
| L1CAM | 1 | intron 1 + 767 | tttgacttccttacatgggt G/A actgtgtgagtcactctgtt | 7588 | | |
| L1CAM | 2 | intron 1 + 862 | gcattgagtcatgtgtatgt G/C tgagtggggctgaatgtaag | 7589 | | |
| L1CAM | 3 | intron 1 + 1332 | cagggatgaaggagcagagc C/T actgaaaggccacacagatg | 7590 | | |
| L1CAM | 4 | intron 4 + 552 | tttccctggggttttccctt T/C gcattccatcctccctgagc | 7591 | | |
| L1CAM | 5 | intron 18 + 147 | agcgacgttatgaaattccc C/A acacttcacatttctataat | 7592 | | |
| L1CAM | 6 | intron 24 + 221 | ctccttagccccccagaggg C/T cccaactttaagagcatact | 7593 | | |
| AANAT | 1 | 5′flanking − 542 | aggggtgcaggatgaggtgt G/T agctggagggcagggggtag | 7594 | | |
| AANAT | 2 | 5′flanking − 263 | ccccccacataagaggtggg C/G ttgtccaagactccgaggga | 7595 | | |
| AANAT | 3 | intron3 39 | cgcccagctccagggaggcc T/A ctgaaaacagaggtcagcca | 7596 | | |
| AANAT | 4 | exon4 150 | cagccagccatacgccaggc C/T gcgctcatgtgcgaggacgc | 7597 | | |
| ARD1 | 1 | intron1 + 317 | ccgtcggtctgctcggcccc C/G etccctcggggctaggcagg | 7598 | | |
| ARD1 | 2 | intron6 + 322 | gctcctcagcatctgctcac G/A ccagggacccacacctctct | 7599 | | |
| ARD1 | 3 | intron6 + 1095 | aaggctccatcctgagacaa A/G aaatccaatataacctgccc | 7600 | | |
| ARD1 | 4 | intron6 + 1179 | aggaggaagacctgtatccc A/G gggacaccctcctccactcc | 7601 | | |
| ARD1 | 5 | intron7 + 159 | cctccaggctgctaggcaga C/T ggcctcctctaaagcccagc | 7602 | | |
| ARD1 | 6 | intron7 + 295 | tgaccagccctgccacccga G/T gagccttggacagaaccctg | 7603 | | |
| ARD1 | 7 | intron7 + 416 | actaccatggaggcccacac G/A acagagcgctgccccttgac | 7604 | | |
| NAT1 | 1 | 3′UTR 215 | aataataataataataataa A/T aaatgtattttaaagatggc | 7605 | | |
| NAT2 | 1 | exon2 867 | cgtgcccaaacctggtaata G/A atcccttactatttagaata | 7606 | | |
| NAT2 | 2 | 3′flank 521 | ccatccatactttgccacaa G/A agaaagaacatgagctttat | 7607 | | |
| NAT2 | 3 | 3′flank 573 | gatttcaaatcctgtagaca C/T ggggtgaattacttttaaaa | 7608 | | |
| NAT2 | 4 | 3′flank 918 | attttctatttgtaaattcc A/G gtatcaaggctatagtttaa | 7609 | | |
| NAT2 | 5 | 3′flank 979 | actattctccctcttcgact C/T gtgatgactataataatctt | 7610 | | |
| NAT2 | 6 | 3′flank 1955 | tacctattgaagtaaaccta C/T gtcatatccacctatttgtt | 7611 | | |
| NAT2 | 7 | 3′flank 2034 | ccactgattcccaaagctag T/G tcattaagaaaacaatgcct | 7612 | | |
| NAT2 | 0 | 3′flank 2201 | cagattactggagggctact G/A tttgctcaccaatacaaatg | 7613 | | |
| NAT2 | 9 | 3′flank 2818 | gggatatttgtctcctttct C/G cccaatgcatattggaaacc | 7614 | | |
| NAT2 | 10 | 3′flank 3237 | atatatattccaattaaaaa A/Δ caaaataaatttccaaaact | 7615 | | |
| NAT2 | 11 | 3′flank 3386 | caacaaagagattttttaaa G/A ctttttaaaacaccagacag | 7616 | | |
| NAT2 | 12 | 3′flank 3660 | cagcactattcacaatagca A/G agatgtgaaatcaatctaaa | 7617 | | |
| NAT2 | 13 | 3′flank 3973 | agcagaaaaaataaataata C/T gtactagacttactacctac | 7618 | | |
| NAT2 | 14 | 3′flank 4029 | caaaacaaacccccataaca T/C gagtttatctatataacaaa | 7619 | | |
| NAT2 | 15 | 3′flank 4118 | ataagattaatatctgcata C/A aaatctttgtttacaactta | 7620 | | |
| NAT2 | 16 | 3′flank 4146 | tgtttacagcttgttatata C/T tgaattatgtctgctccccc | 7621 | | |
| NAT2 | 17 | 3′flank 4279 | ttaatctgataaaattggtg G/C ctttataagaaaaagaaaag | 7622 | | |
| NAT2 | 18 | 3′flank 4323 | ttgctctctccccagtgcag T/G taccaaggaaagaccatgta | 7623 | | |
| NAT2 | 19 | 3′flank 4446 | tcaattaactttatctgcaa T/C tctagaatcaggcaatactc | 7624 | | |
| NAT2 | 20 | 3′flank 4462 | gcgattctggaatcaggcaa T/C actccatttcataaaacaga | 7625 | | |
| NAT2 | 21 | exon2 + 255 | atgttaggagggtattttta C/T atccctccaattaacaaata | 7626 | | |
| NAT2 | 22 | 5′flank − 2053 | ctaaattaaaacattttaat T/C ccaggtgtcaggtttccaac | 7627 | | |
| NAT2 | 23 | 5′flank − 1299 | gaatcaccaatgcaagagat A/G taacaatgaacccaagacac | 7628 | | |
| NAT2 | 24 | 5′flank − 1145 | ctgtagaacacaaggatatt C/T aaagacagtttgtacatacc | 7629 | | |
| NAT2 | 25 | 5′flank − 1036 | ccttcccacagagtcccgag T/A tcatgtgacagcatgccaga | 7630 | | |
| NAT2 | 26 | 5′flank − 94 | aaaaatttgctaagaaattc G/A cagaagcaacctaaggccct | 7631 | | |
| NAT2 | 27 | 5′flank − 643 | atgtttatattttatattaa T/C attaatgtaaataaaaattt | 7632 | | |
| ABCB2 | 1 | 5′flanking − 673 | agctaagagtcaaagcaccc G/C ctttttccaccagcctcgcg | 7633 | | |
| ABCB2 | 2 | 5′flanking − 646 | ccaccagcctcgcgtgcctg T/C tcccttcacggacactctag | 7634 | | |
| ABCB2 | 3 | 5′flanking − 563 | ttgcaagcgctggctgctac A/G ggcgacctccctgcgctccc | 7635 | | |
| ABCB2 | 4 | 5′flanking − 236 | gctttgcgcgcggcgctaac C/T tgtgtagggcagatctgccc | 7636 | | |
| ABCB2 | 5 | Intron3 + 408 | aaggaaactgaggccaagac C/T ctaaatgctgaaactgcaca | 7637 | | |
| ABCB2 | 6 | Exon4 + 153 | ccctcaccatggtcaccctg A/G tcaccctgcctctgcttttc | 7638 | | |
| ABCB2 | 7 | Intron4 + 289 | gtatttctttagcatccaag G/T ggcatagctgtgtctctttc | 7639 | | |
| ABCB2 | 8 | Intron4 + 291 | atttctttagcatccagggg C/G catagctgtgtctctttctc | 7640 | | |
| ABCB2 | 9 | Intrpn5 − 63 | ttccttcaggttaatgactg C/T ggttctttgtgtcccctcca | 7641 | | |
| ABCB2 | 10 | Intron7 − 185 | gtctctgcccttgtctttgc C/T gcttcttctatctctactcc | 7642 | | |
| ABCB2 | 11 | 3′flanking + 71 | agcgcacttttcagctgcgg C/A tgtctcctcttttatcatcc | 7643 | | |
| ABCB2 | 12 | 3′flanking + 129 | aactgcatcaccttttccct T/C aagctttttaattcctatga | 7644 | | |
| ABCB2 | 13 | 3′flanking + 455 | cattcagggaggcccaggtc C/A tgtgacgtcgacagttgctg | 7645 | | |
| ABCB3 | 1 | intron3 + 8 | tctcctttggcaggtaggtg C/A tgggcagctgggtccatttg | 7646 | | |
| ABCB3 | 2 | intron4 + 104 | cttcacccgtatgccaggac C/T tggggatgcttttctcttgt | 7647 | | |
| ABCB3 | 3 | intron10 + 219 | gcagcactggtgctccctcc A/G tgggcagccccgtcaggtcc | 7648 | | |
| ABCB3 | 4 | intron11 + (317-319) | atggtgcccaggtggatgtg GTG/Δ tccatctcattcctgtcttt | 7649 | | |
| ABCB3 | 5 | exon12 + 19 | agctgcaggactggaattcc T/C gtggggatcgcacagtgctg | 7650 | | |
| ABCB3 | 6 | exon12 + (356-357) | aggtggggtggcgtggggtc GG/TGGTGGGGTGGA ggctgtctgtgtcc | 7651 | | |
| GSTM3 | 1 | 5′flanking − 144 | ccaacgccggcattagtcgc C/G cctgcgcacggccctgtgga | 7652 | | |
| GSTM3 | 2 | intron 7 + 165 | agcctaacttctataccttg A/G aggcactgtctacaaaaaaa | 7653 | | |
| GSTM3 | 3 | intron 7 + 257 | ctgttgcactgggtggggtc C/G ttataagattggtgtatttt | 7654 | | |
| GSTM3 | 4 | exon 5 + 51 | cccagtggggcaacaagcct A/G tatgctgagcaggaggcaga | 7655 | | |
| GSTM4 | 1 | intron 4 + 67 | ttggctggattggggtgcta C/G gctcacagtgagtctgtgtt | 7656 | | |
| GSTM4 | 2 | intron 7 + 77 | gatgctttcccagtcctgga T/C ctgcataaagaataacttgc | 7657 | | |
| GSTM4 | 3 | intron 7 + 80 | gctttcccagtcctggatct C/A cataaagaataacttgcatt | 7658 | | |
| ALDH7 | 1 | intron1 + 464 | catgaatgactctgggaaag A/G atcattcttagcaatggact | 7659 | | |
| ALDH7 | 2 | intron1 + 2269 | aaatggaatccaaacagcaa C/G agacatcccctcaccggtca | 7660 | | |
| ALDH7 | 3 | intron2 + 1349 | actgagcttctgccaccggc C/G gcctgccggccttcatgaga | 7661 | | |
| ALDH7 | 4 | intron2 + 1820 | tccgtgtgggaggcaccttc C/G cccagcctcagtggctagga | 7662 | | |
| ALDH7 | 5 | intron2 + 2046 | aacctcaggcgctgcctcag C/G cagggagccagcctggcccc | 7663 | | |
| ALDH7 | 6 | intron2 + 2939 | aagcacgcactgaacatgga C/A tgagtgagtgaaccaatgaa | 7664 | | |
| ALDH7 | 7 | intron3 + 7 | tgcccaggaacctggtgagc C/T ggccgggctgaggcgggcag | 7665 | | |
| ALDH7 | 8 | intron4 + 36 | gccccttccggtcacccttc T/C ccgctcgaggcctcagggcc | 7666 | | |
| ALDH7 | 9 | intron6 + (116-117) | attctcctctctctctctct CT/Δ ggacaaggctgggagcagtc | 7667 | | |
| ALDH7 | 10 | intron6 + 263 | cagaccctcatacgtgaccc C/G gctgccccccaggctcttag | 7668 | | |
| HMG17L1 | 1 | 3′untranslated + 864 | ctttctgatttttgatagtc C/G gttgaagaagggagtttgaa | 7669 | | |

In some embodiments, a drug-metabolizing enzyme is at least one of the following: epoxide hydrolase, methyltransferase, N-acetyltransferase, sulfotransferase, quinone oxidereductase, glutathione S-transferase, UDP-glycosyltransferase, aldehyde dehydrogenase, alcohol dehydrogenase, esterase, NDUF, cytochrome P450 (CYP) and ATP-binding cassette.

The present invention relates to a method for detecting a genetic polymorphism in a test subject using the genetic polymorphism data related to a drug metabolizing enzyme. The present invention analyzes the effectiveness, safety and strength of drugs metabolized by a drug metabolizing enzyme. The relationship between a disease and the drug to be evaluated is based on the results of the analysis. The genetic polymorphism data for the drug metabolizing enzyme is different for each patient with a given disease. Therefore, the effectiveness and safety of a specific drug depends on drug metabolism in the presence of certain genetic polymorphism data and the side effects in the presence of certain genetic polymorphism data. As a result, a physician can determine whether a certain drug should be used by a certain patient and can tailor drugs for use by a certain patient based on the genetic polymorphism data (so-called “made-to-order” treatments).

“Drug metabolizing enzymes” refer to a group of enzymes that catalyze in vivo structural changes in exogenous materials including drugs. When used for clinical purposes, the group of metabolizing enzymes includes some endogenous materials. Because drug-metabolizing enzymes absorb, metabolize and secrete drugs, the polymorphism of an enzyme depends on the amount of enzyme expressed (transcription and translation) and the amount of activity. As a result, there are blood serum concentrations of both unchanged materials and metabolites.

Drug metabolizing enzymes expressed by the genes that are targeted for genetic polymorphism analysis in the present invention include, but are not limited to the following classes of enzymes:

Epoxide hydrolases

Methyltransferases

N-acetyltransferases

Sulfotransferases

Quinone oxidereductases

Glutathione S-transferases

UDP-glycosyltransferases

Aldehyde dehydrogenases

Alcohol dehydrogenases

Esterases

Ubiquinone dehydrogenases: NDUF

Cytochrome P450s (CYPs)

ATP-binding cassettes

ATP-binding cassettes/Transporters

Examples and descriptions of these enzymes are provided below.

(1) Epoxide hydrolases are enzymes that hydrolyze epoxide using a trans-cleavage mechanism to produce 1,2-glycol. Examples include microsomal epoxide hydrolase 1 and cytoplasmic epoxide hydrolase 2.

(2) Methyltransferases are enzymes that catalyze transmethylation in amino groups, hydroxyl groups and thiol groups. Examples include the following.

Catechol-O-methyltransferase

Vitamin-N-methyltransferase

Phenylethanolamine-N-methyltransferase

Phosphatidylethanolamine-N-methyltransferase

Nicotinamide-N-methyltransferase

Acetylserotonin-O-methyltransferase

Thiopurine S-methyltransferase

(3) N-acetyltransferases are enzymes that catalyze transacetylation in amino groups, sulfonamide groups and hydrazine groups. Examples include the following.

Arylamine-N-acetyltransferase 1, 2

Arylalkylamine-N-acetyltransferase

N-acetyltransferase homologues of saccharomyces cerevisiae

LI intracellular adhesion molecules

(4) Sulfotransferases are enzymes that contribute to sulfate conjugation and catalyzes trans-sulfonylation in phenols, steroids, arylamines and biliary acid. Examples include the following.

Sulfotransferase 1A1, 1A2, 1A3, 1C1, 1C2, 2A1, 2B1

Thyroid hormone sulfotransferase

Tyrosyl protein sulfotransferase 1, 2

Sulfotransferase-opening protein 3

Estrogen sulfotransferase

Cerebroside sulfotransferase

HNK-sulfotransferase 1

Carbohydrate sulfotransferase 2, 4, 5

Carbohydrate sulfotransferase 1, 3

(5) Quinone oxidereductases are enzymes that catalyze the reduction of quinones such as o-quinone and p-quinone. Examples include the following.

NAD(P)H: Quinone oxidereductase 1

NRH: Quinone oxidereductase 2

Quinone oxidereductase homologues

p53-induced gene 3 (PIG3) of a quinone oxide transferase homologue

(6) Glutathione S-transferases are enzymes that catalyze the conjugation of glutathione. Examples include the following.

Glutathione S-transferase Mu1, Mu2, Mu3, Mu4, Mu5

Glutathione S-transferase Z (zeta)

Glutathione S-transferase P (pi)

Glutathione S-transferase 1 T1 (zeta)

Glutathione S-transferase 1 Theta 1, Theta 2

Microsomal Glutathione S-transferase 1

Microsomal Glutathione S-transferase 1-1

Microsomal Glutathione S-transferase 2, 3

Microsomal Glutathione S-transferase Ha Subunit 1, 2

Microsomal Glutathione S-transferase A3, A4

Glutathione S-transferase A1, A4

Glutathione S-transferase M1, M2, M3, M4

(7) UDP-glycosyltransferases are enzymes that catalyze the contribution of glucuronic acid to functional groups such as hydroxyl groups, carboxyl groups, amino groups and thiol groups after their introduction in the 1 st drug metabolism route. Examples include the following.

UDP-glycosyltransferase 1

UDP-glycosyltransferase 1 Family Polypeptide A1

UDP-glycosyltransferase 2 Family Polypeptide A1, B7, B10, B4, B11, B15, B17

UDP-glycosyltransferase 8

Dolichyl-diP-oligosaccharide protein glycosyl transferase

(8) Aldehyde dehydrogenases are enzyme that converts aldehydes into carboxylic acids. Examples include Aldehyde dehydrogenase 1 through 10.

Aldehyde dehydrogenase 1 family member A1, A2, A3

Aldehyde dehydrogenase 1 family member B1

Formyltetrahydroforate dehydrogenase

Aldehyde dehydrogenase 2

Aldehyde dehydrogenase 3 family member A1, A2

Aldehyde dehydrogenase 3 family member B1, B2

Aldehyde dehydrogenase 5 family member A1

Aldehyde dehydrogenase 6 family member A1

Aldehyde dehydrogenase 8 family member A1

Aldehyde dehydrogenase 9 family member A1

(9) Alcohol dehydrogenases are enzymes that convert alcohols into aldehydes or ketones. Examples include the following.

Alcohol dehydrogenase 1 through 7

Hydroxy-CoA-dehydrogenase

Short-chain alcohol dehydrogenase family genes

(10) Esterases are enzymes that hydrolyze some esters. Examples include the following.

Arylacetoamide deacetylase

Granzyme A

Granzyme B

Interleukin 17

Ubiquitin carboxyl-terminal esterase L1, 3

Carboxyl esterase 1

Lipase A

Esterase D-formylglutathione hydrolase

Carboxylester lipase

Dolichyl-diphosphooligosaccharide-protein glycosyltransferase (DDOST)

Neuropathy target esterase

(11) Ubiquinone dehydrogenases (NDUF) are enzymes that support energy metabolism, e.g., as in the mitochondrial respiratory chain. Examples include NADH ubiquinone dehydrogenase 1a Subunit 1 through 10.

NADH-dehydrogenase (ubiquinone) 1α-subcomplex 1 through 3 and 5 through 10

NADH-dehydrogenase (ubiquinone) 1α/β-subcomplex 1

NADH-dehydrogenase (ubiquinone) 1β-subcomplex 3, 5, 7

NADH-dehydrogenase (ubiquinone) Fe—S protein 1, 3, 4, 5, 6, 8

NADH-dehydrogenase (ubiquinone) flavoprotein 1 through 3

(12) Cytochrome P450s (CYPs) are enzymes that regulate 1st drug metabolism and introduce oxygen atoms to the drug. Examples include Cytochrome P450 (CYP) 1A1, CYP 1A2, CYP1B1, CYP 2A6, CYP 2B6, CYP 2C8, CYP 2C18, CYP 2C9, CYP 2C19, CYP 2E1, CYP 2D6, CYP 2E1, CYP 2F1, CYP 3A3, CYP 3A4, CYP 3A5, CYP 3A7, CYP 3A43, CYP 4A1, CYP 4B1, CYP 4F2, CYP 4F3, CYP 4F8, CYP 11B1, CYP 11B2, CYP 17, CYP 19, CYP 21A2, CYP 21A1, CYP 27B1 and CYP 27.

(13) ATP-binding cassettes absorb the drug and adjust the interstitial concentration with a transporter. Examples include the following.

ATP-Binding Cassette Subfamily A Members 1 through 6, 8

ATP-Binding Cassette Subfamily A Members 1, 4, 7, 8

ATP-Binding Cassette Subfamily B Members 1 through 11

ATP-Binding Cassette Subfamily B Members 1, 4, 7, 8, 9, 10, 11

ATP-Binding Cassette Subfamily C Members 1 through 6, 8 through 10

ATP-Binding Cassette Subfamily C Members 1, 2, 3, 4, 5, 7, 8, 9

ATP-Binding Cassette Subfamily D Members 1 through 4

ATP-Binding Cassette Subfamily D Members 1, 3, 4

ATP-Binding Cassette Subfamily E Members 1

ATP-Binding Cassette Subfamily F Members 1 through 3

ATP-Binding Cassette Subfamily F Member 1

ATP-Binding Cassette Subfamily G Members 1

ATP-Binding Cassette Subfamily G Members 1, 2, 4, 8

Organic anion transporters 1, 2, 3

Organic anion transporter polypeptides 1, 2, 8

Transporter 1 ATP-binding cassette subfamily B

Transporter 2 ATP-binding cassette subfamily B

SLC22A4 solute carrier family 22 (organic cation transporter) member 4

SLC22A5 solute carrier family 22 (organic cation transporter) member 5

SLC22A1 solute carrier family 22 (organic cation transporter) member 1

SLC22A2 solute carrier family 22 (organic cation transporter) member 2

SLC10A2 solute carrier family 10 (sodium/bile acid cotransporter family) member 2

SLC15A 1 solute carrier family 15 (oligopeptide transporter) member 1

(14) Other enzymes include gamma glutamyl transferase 1, transglutaminase 1 and dihydropyrimidine dihydrogenase.

Genetic polymorphism data relating to DMEs can be obtained using any general genetic polymorphism detection method. Examples include, but are not limited to, PCR or other amplification methods, hybridization methods using an allele-specific oligonucleotide matrix (e.g., TAQMAN PCR method, INVADER assay method), primer extension reaction methods, sequencing methods, MALDI-TOF/MS methods and the DNA chip methods (e.g., microarrays). Examples of detection methods that are applicable to analysis of the DME associated polymorphisms of the present invention include but are not limited to those listed below.

1. Direct Sequencing Assays

In some embodiments of the present invention, variant sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the SNP or mutation of interest) is sequenced using any suitable method, including but not limited to manual sequencing using radioactive marker nucleotides, or automated sequencing. The results of the sequencing are displayed using any suitable method. The sequence is examined and the presence or absence of a given SNP or mutation is determined.

2. PCR Assay

In some embodiments of the present invention, variant sequences are detected using a PCR-based assay. In some embodiments, the PCR assay comprises the use of oligonucleotide primers that hybridize only to the variant or wild type allele (e.g., to the region of polymorphism or mutation). Both sets of primers are used to amplify a sample of DNA. If only the mutant primers result in a PCR product, then the patient has the mutant allele. If only the wild-type primers result in a PCR product, then the patient has the wild type allele.

3. Fragment Length Polymorphism Assays

In some embodiments of the present invention, variant sequences are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I [Third Wave Technologies, Madison, Wis.] enzyme). DNA fragments from a sample containing a SNP or a mutation will have a different banding pattern than wild type.

a. RFLP Assay

In some embodiments of the present invention, variant sequences are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are generally separated by gel electrophoresis and may be visualized by ethidium bromide staining. The length of the fragments is compared to molecular weight markers and fragments generated from wild-type and mutant controls.

b. CFLP Assay

In other embodiments, variant sequences are detected using a CLEAVASE fragment length polymorphism assay (CFLP; Third Wave Technologies, Madison, Wis.; See e.g., U.S. Pat. Nos. 5,843,654; 5,843,669; 5,719,208; and 5,888,780; each of which (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition or low, medium, or high stringency conditions. Unbound probe is removed and the presence of binding is detected by visualizing the labeled probe.

b. Detection of Hybridization Using “DNA Chip” Assays

In some embodiments of the present invention, variant sequences are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding,

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

DNA probes unique for the SNP or mutation of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.

C. Enzymatic Detection of Hybridization

In some embodiments of the present invention, hybridization is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with a fluorescent dye that is quenched by a second dye or other quenching moiety. Upon cleavage, the de-quenched dye-labeled product may be detected using a standard fluorescence plate reader, or an instrument configured to collect fluorescence data during the course of the reaction (i.e., a “real-time” fluorescence detector, such as an ABI 7700 Sequence Detection System, Applied Biosystems, Foster City, Calif.).

The INVADER assay detects specific mutations and SNPs in unamplified genomic DNA. In an embodiment of the INVADER assay used for detecting SNPs in genomic DNA, two oligonucleotides (a primary probe specific either for a SNP/mutation or wild type sequence, and an INVADER oligonucleotide) hybridize in tandem to the genomic DNA to form an overlapping structure. A structure-specific nuclease enzyme recognizes this overlapping structure and cleaves the primary probe. In a secondary reaction, cleaved primary probe combines with a fluorescence-labeled secondary probe to create another overlapping structure that is cleaved by the enzyme. The initial and secondary reactions can run concurrently in the same vessel. Cleavage of the secondary probe is detected by using a fluorescence detector, as described above. The signal of the test sample may be compared to known positive and negative controls.

In some embodiments, hybridization of a bound probe is detected using a TAQMAN assay (PE Biosystems, Foster City, Calif.; See e.g., U.S. Pat. Nos. 5,962,233 and 5,538,848, each of which is herein incorporated by reference). The assay is performed during a PCR reaction. The TAQMAN assay exploits the 5′-3′ exonuclease activity of DNA polymerases such as AMPLITAQ DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labeled antibody specific for biotin).

5. Other Detection Assays

Additional detection assays that are produced and utilized using the systems and methods of the present invention include, but are not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884 and 6,183,960, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Bamay Proc. Natl. Acad. Sci USA 88, 189-93 (1991)); and sandwich hybridization methods (e.g., U.S. Pat. No. 5,288,609, herein incorporated by reference in its entirety).

6. Mass Spectroscopy Assay

In some embodiments, a MassARRAY system (Sequenom, San Diego, Calif.) is used to detect variant sequences (See e.g., U.S. Pat. Nos. 6,043,031; 5,777,324; and 5,605,798; each of which is herein incorporated by reference). DNA is isolated from blood samples using standard procedures. Next, specific DNA regions containing the mutation or SNP of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization—Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The Spec troTYPER software then calculates, records, compares and reports the genotypes at the rate of three seconds per sample.

In some embodiments, the present invention provides an oligonucleotide comprising a DME related sequence, or a complement of a DME-related sequence. In preferred embodiments, an oligonucleotide of the present invention comprises a sequence or a complement of a sequence selected from the group consisting SEQ ID NOs. 1-3360 and 3361-7669, or a substantially similar sequence.

In some embodiments, an oligonucleotide probe or oligonucleotide primer is created so the 5′ terminus, 3′ terminus or central base contains the genetic polymorphism site. In some preferred embodiments, an oligonucleotide is created comprising at least 13 contiguous bases of a sequence selected from SEQ ID NOs 1 through 3360 and 3361-7669, or the complement thereto, and further comprising the 21^{st} nucleotide of the sequence selected from SEQ ID NOs 1 through 3360 and 3661-7669, or the complement thereto.

In some embodiments, an oligonucleotide of the present invention flanks or is adjacent to a polymorphic site, such that the presence of the polymorphism can be detected by modification of the oligonucleotide in a manner dependent on the presence or absence of the polymorphism.

In some embodiments, the present invention provides kits comprising one or more of the components necessary for practicing the present invention. For example, the present invention provides kits for storing or delivering the enzymes of the present invention and/or the reaction components necessary to practice a cleavage assay (e.g., the INVADER assay). The kit may include any and all components necessary or desired for the enzymes or assays including, but not limited to, the reagents themselves, buffers, control reagents (e.g., tissue samples, positive and negative control target oligonucleotides, etc.), solid supports, labels, written and/or pictorial instructions and product information, inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered. For example, a first container (e.g., box) may contain an enzyme (e.g., structure specific cleavage enzyme in a suitable storage buffer and container), while a second box may contain oligonucleotides (e.g., INVADER oligonucleotides, probe oligonucleotides, control target oligonucleotides, etc.). In some embodiments one or more the reaction components may be provided in a predispensed format (i.e., pre-measured for use in a step of the procedure without re-measurement or re-dispensing). In some embodiments, selected reaction components are mixed and predispensed together. In preferred embodiments, predispensed reaction components are predispensed and are provided in a reaction vessel (including but not limited to a reaction tube or a well, as in, e.g., a microtiter plate). In particularly preferred embodiments, predispensed reaction components are dried down (e.g., desiccated or lyophilized) in a reaction vessel.

Examples of genetic polymorphism data (especially the SNP data) that can be used in the method of the present invention are shown in Table 1.

In Table 1, the name of the gene encoding the drug metabolizing enzyme is recorded in the gene name column. The base in capital letters is the SNP data in the sequence column. Two bases separated by a forward slash indicate the SNP of homo and hetero bases. For example, A/G indicates a homo allele A/A and G/G as well as a hetero allele A/G. The sequences in this table have 20 bases before and after the SNP. Here, the base in parentheses, for example the 26th (T) in ABCB4, indicates a polymorphism with an inserted base, and D, such as the 10th spot in NAT2, indicates a polymorphism with a deleted base. In Sequence No. 674, n is VNTR and (cctgy)x, where x is an integer between 1 and 50, indicates a repeated sequence. The bases with numbers in parentheses indicate the number of times they are repeated. For example, “(T) 9-12” in Sequence No. 1552 (ABCB11 No. 55 in Table 1) indicates T is repeated 9 to 12 times.

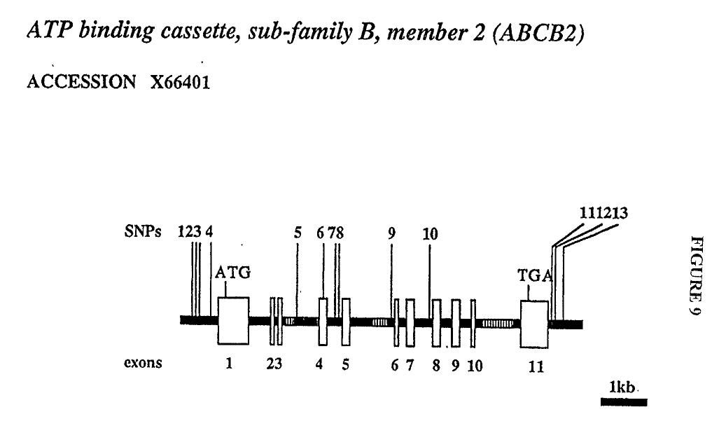

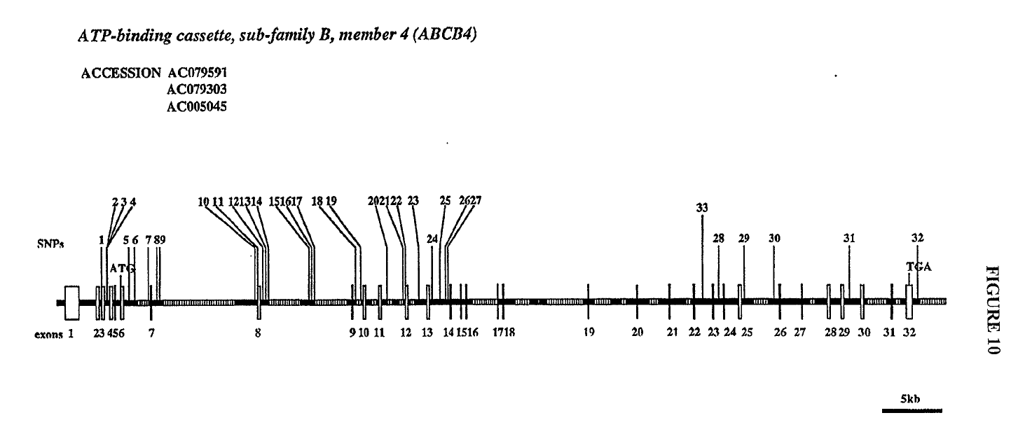

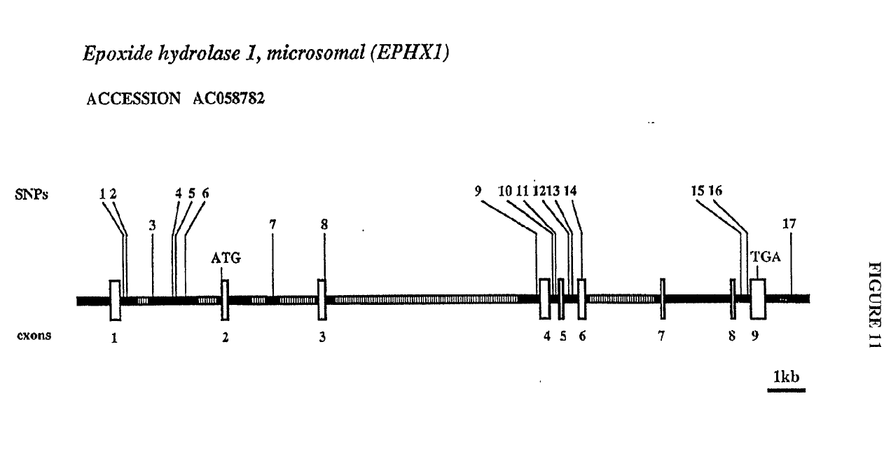

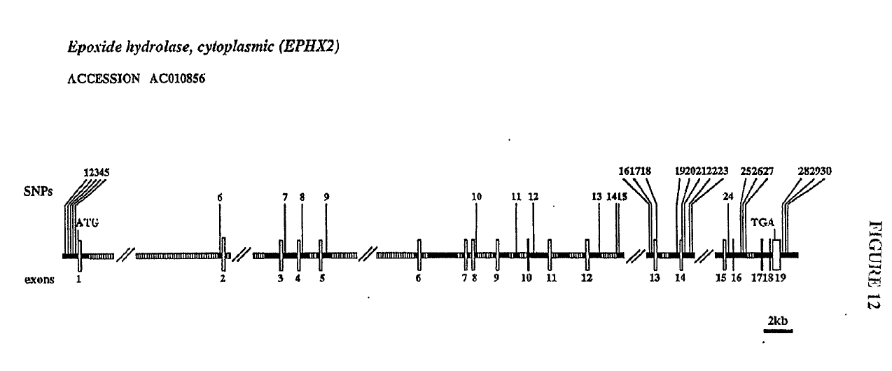

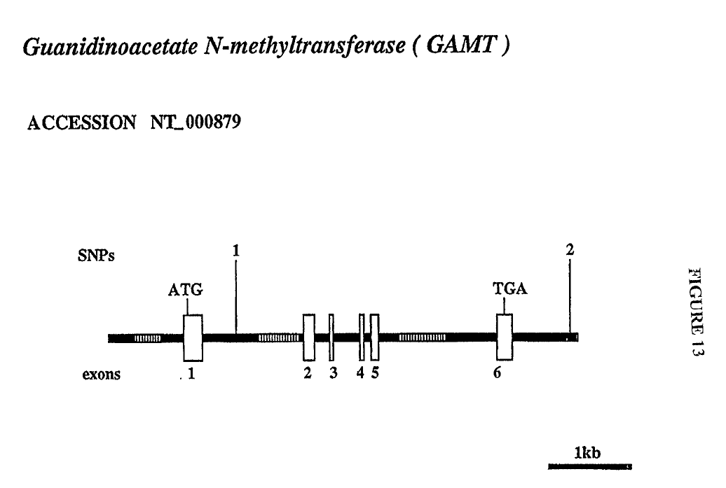

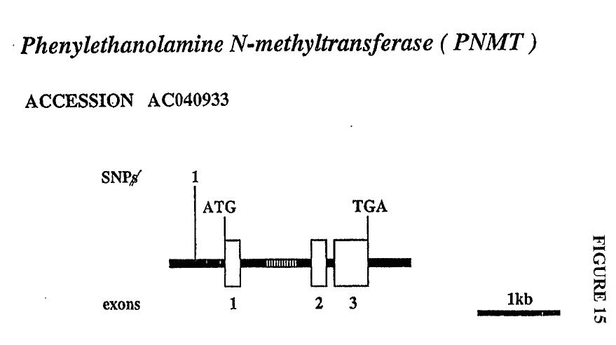

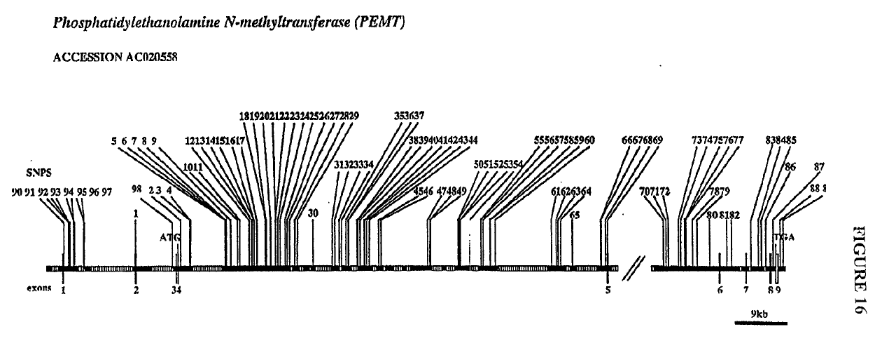

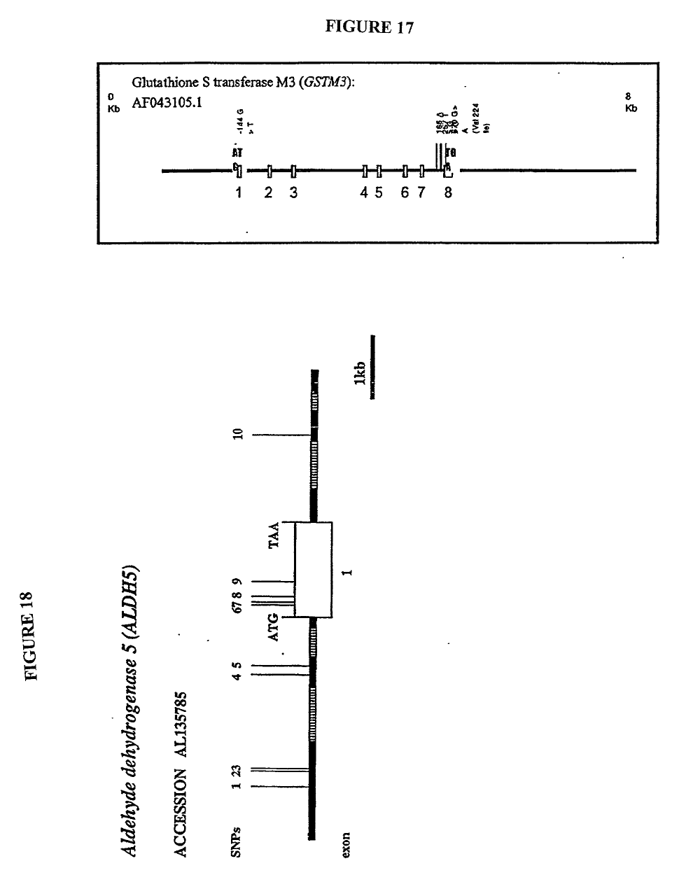

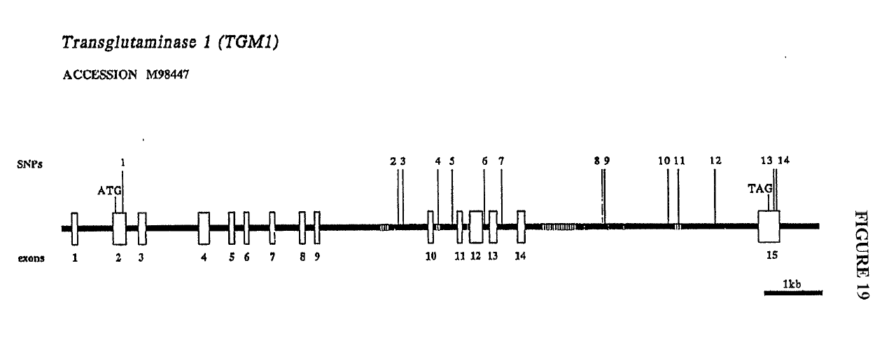

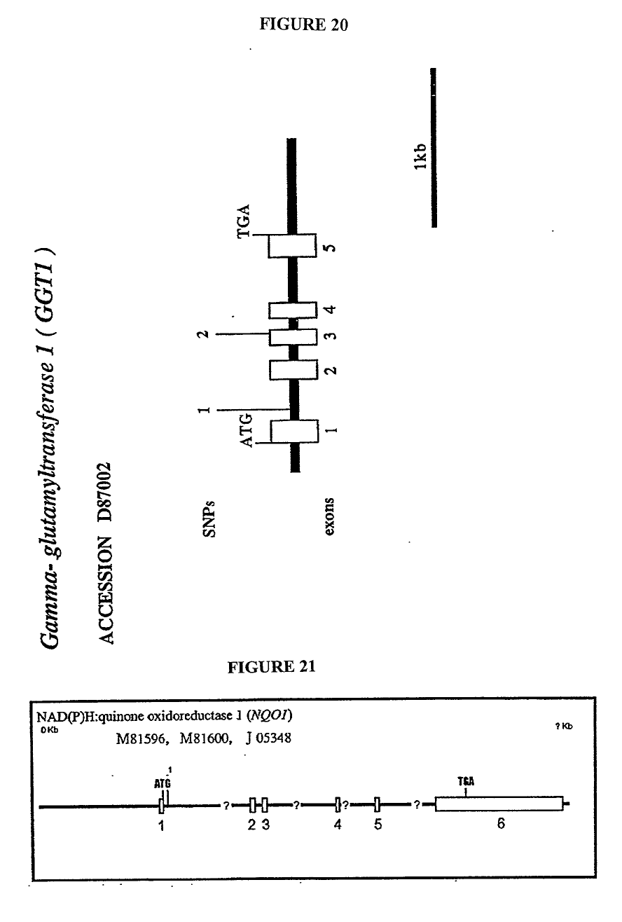

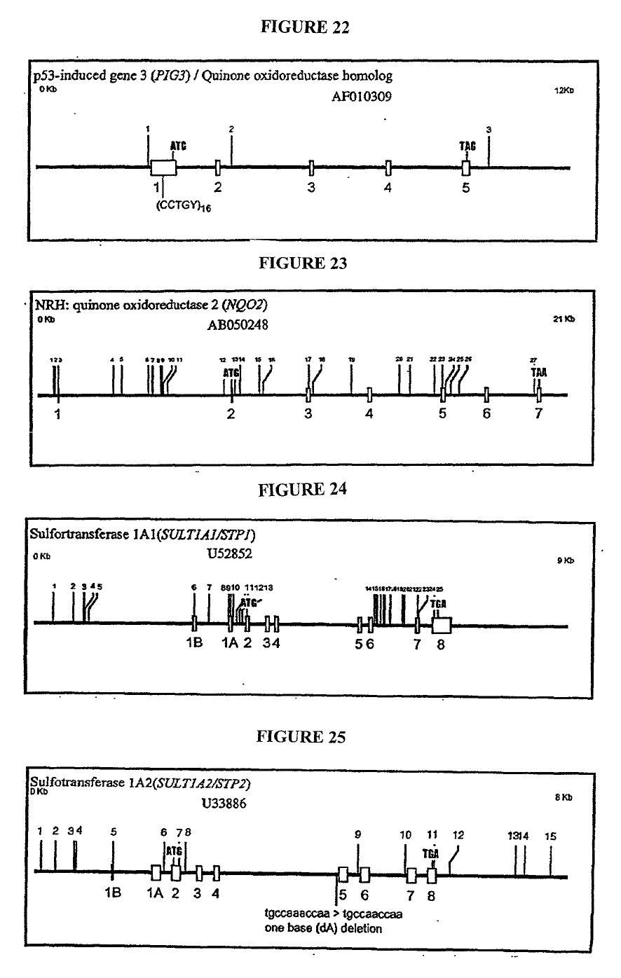

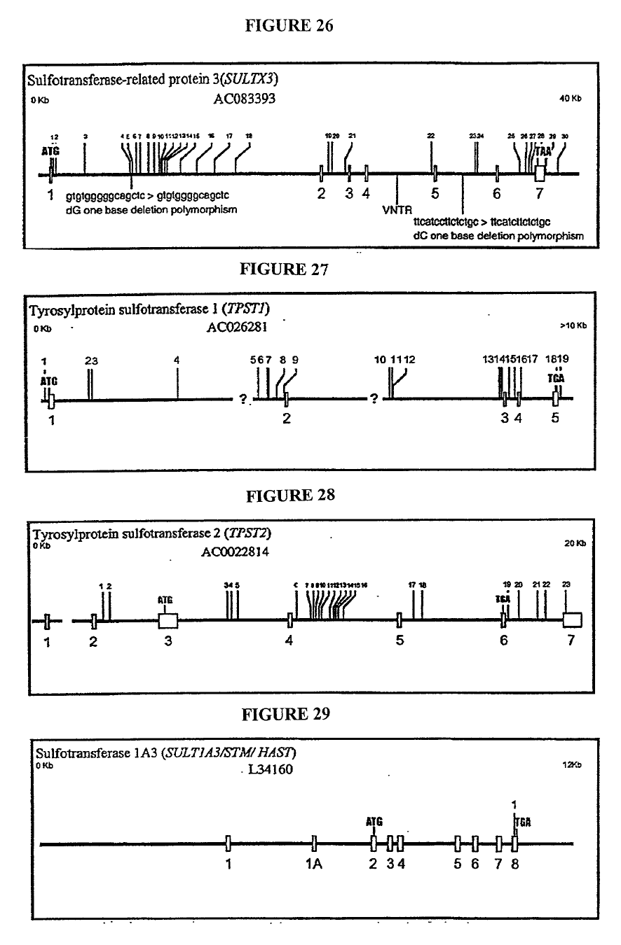

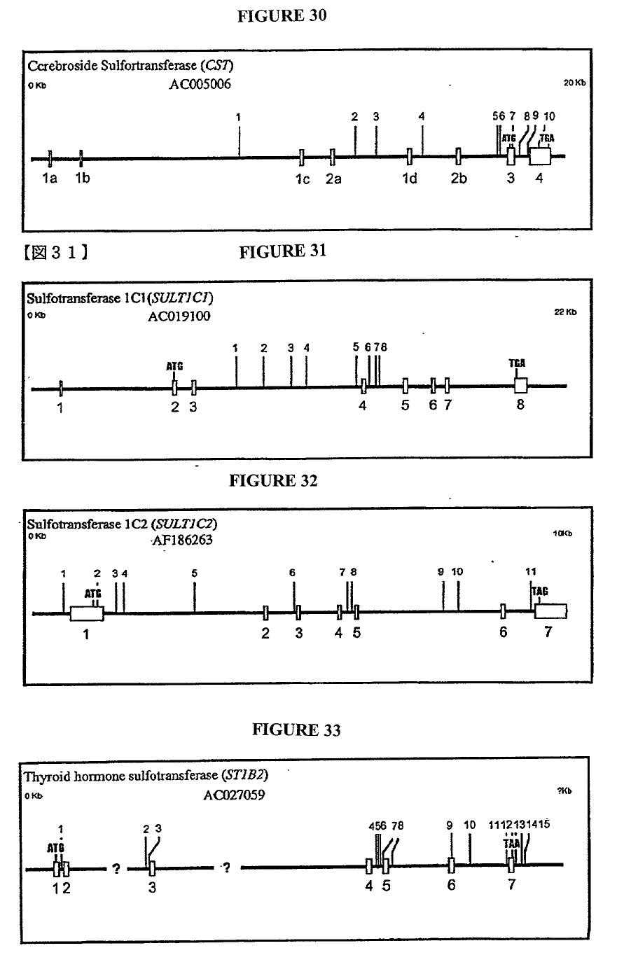

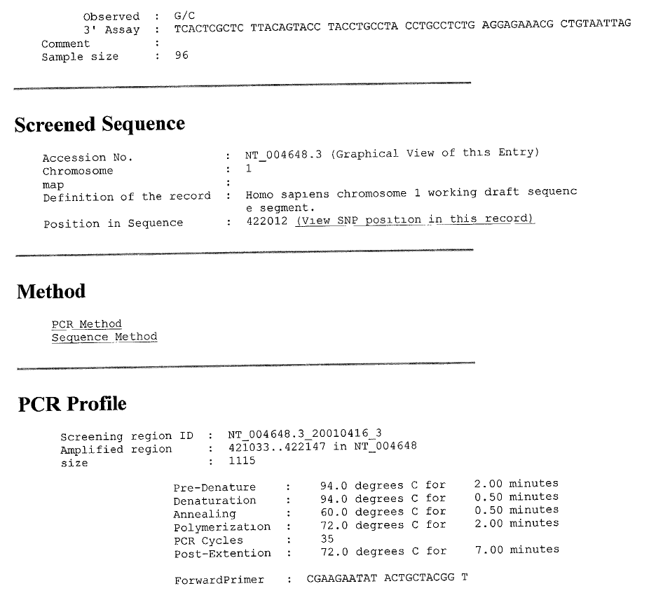

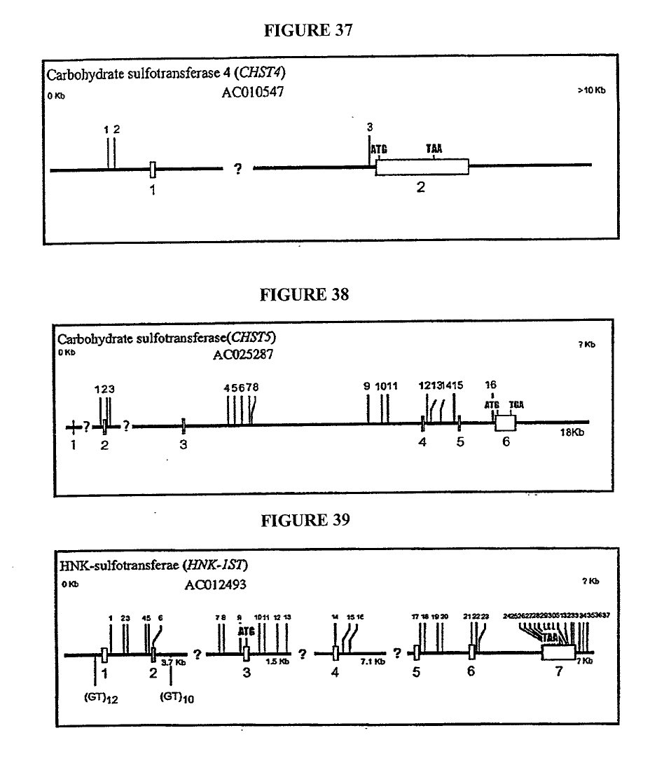

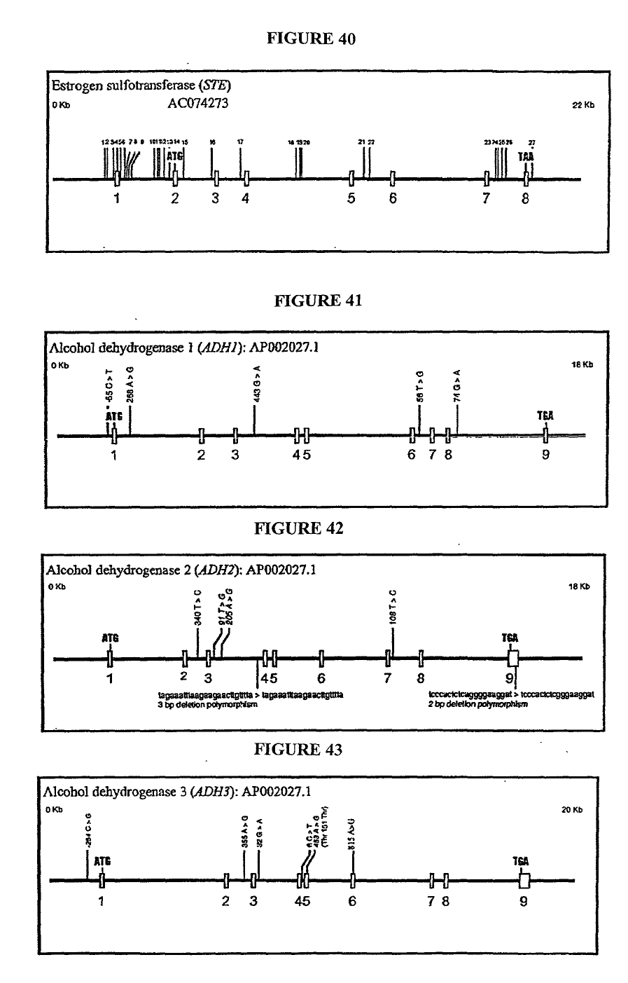

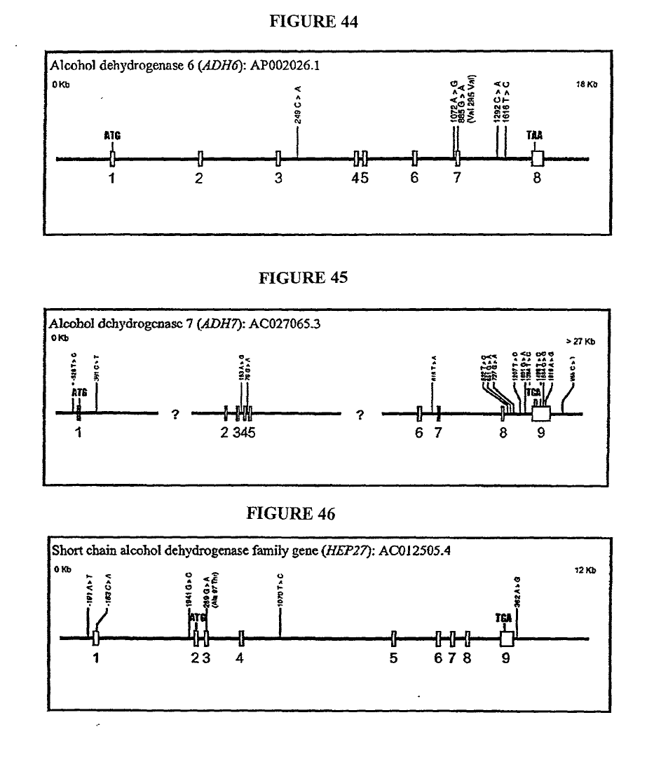

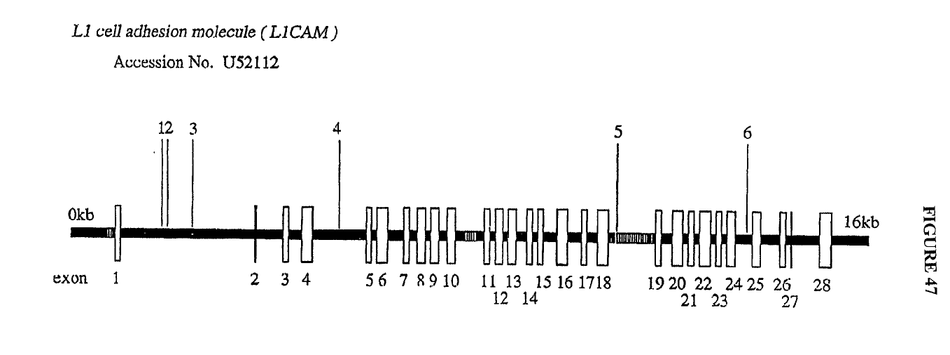

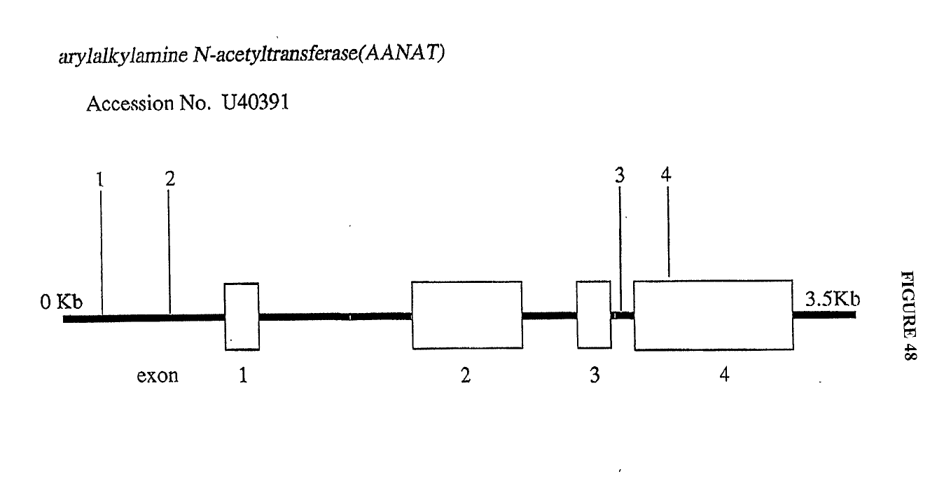

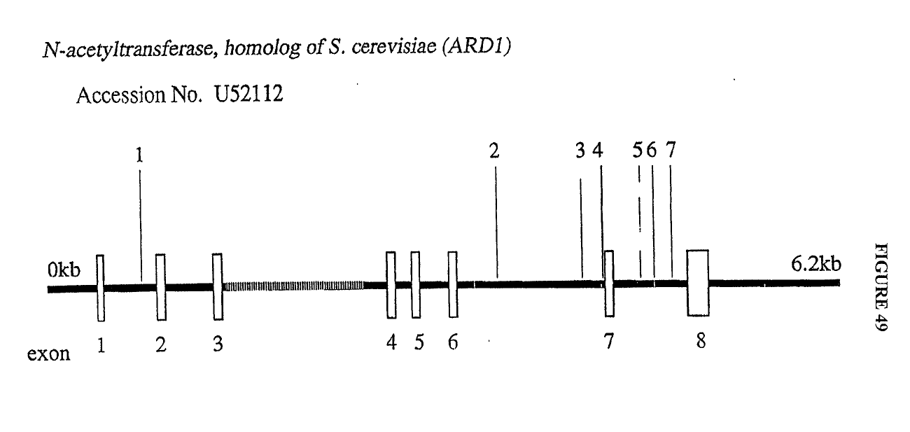

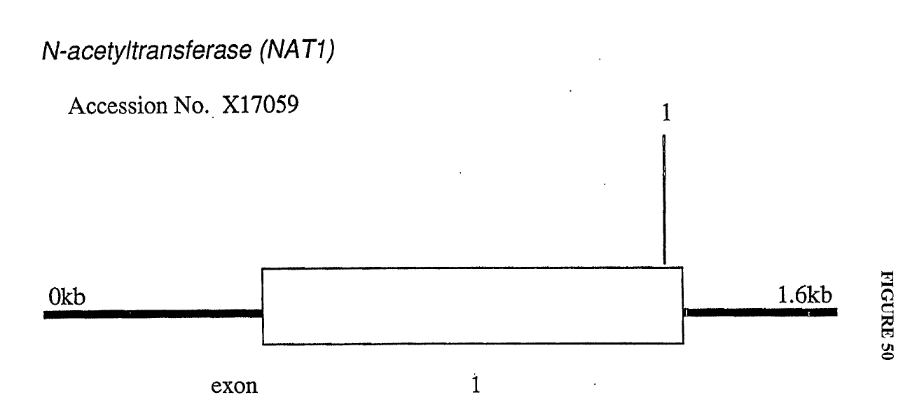

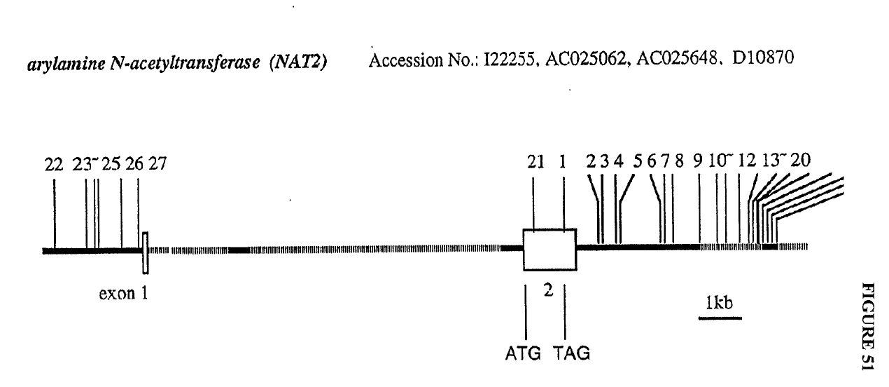

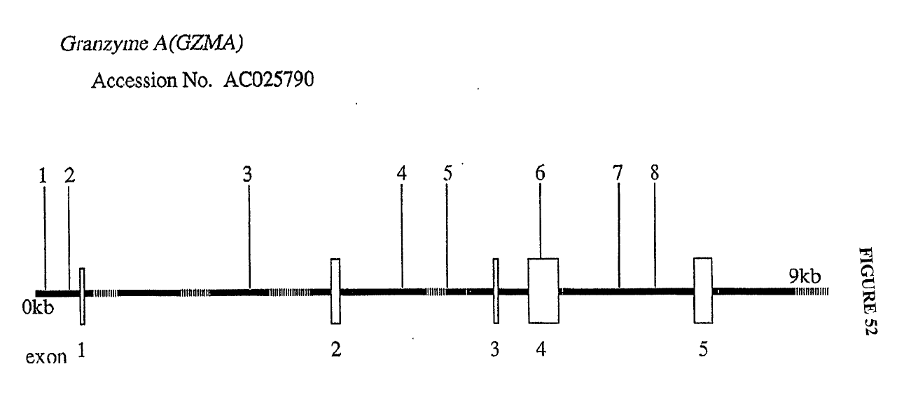

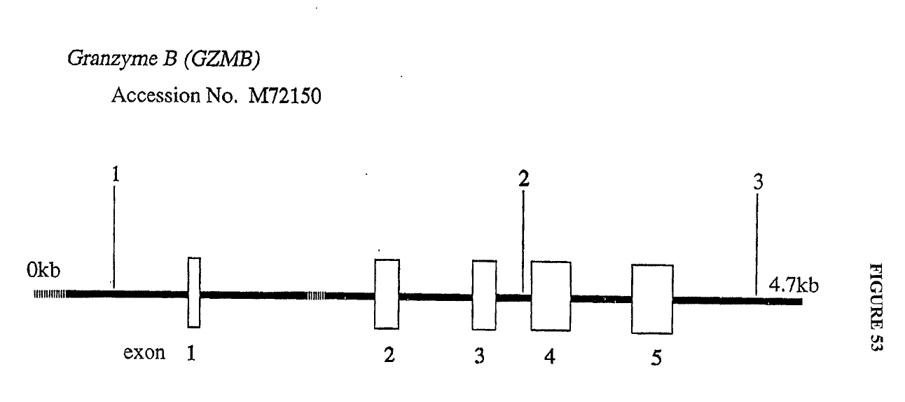

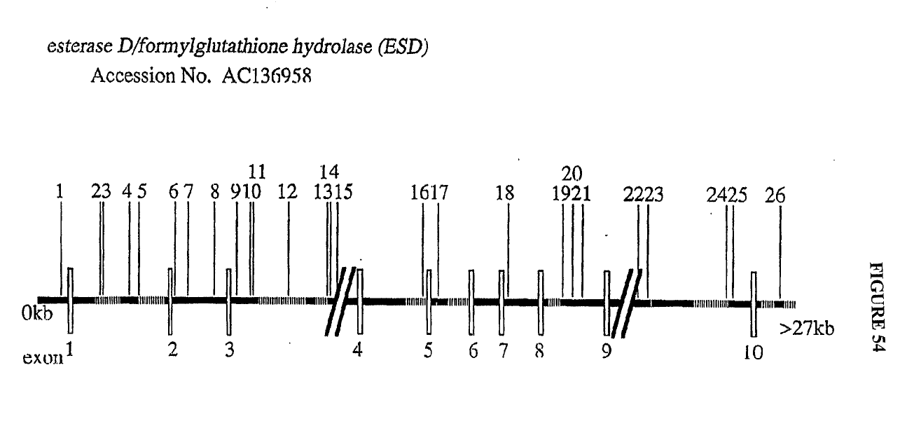

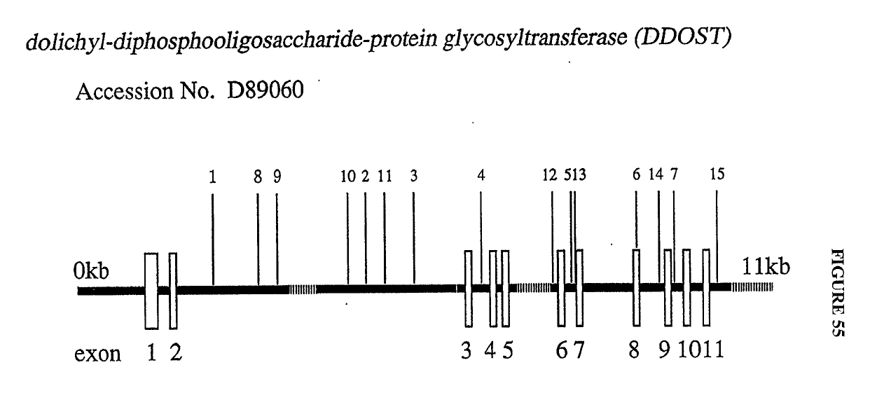

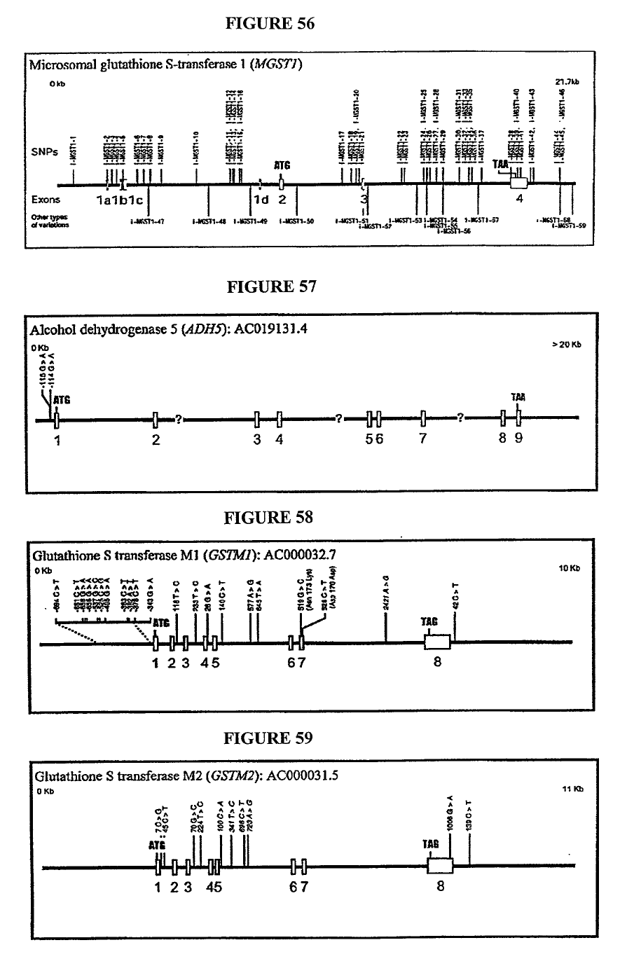

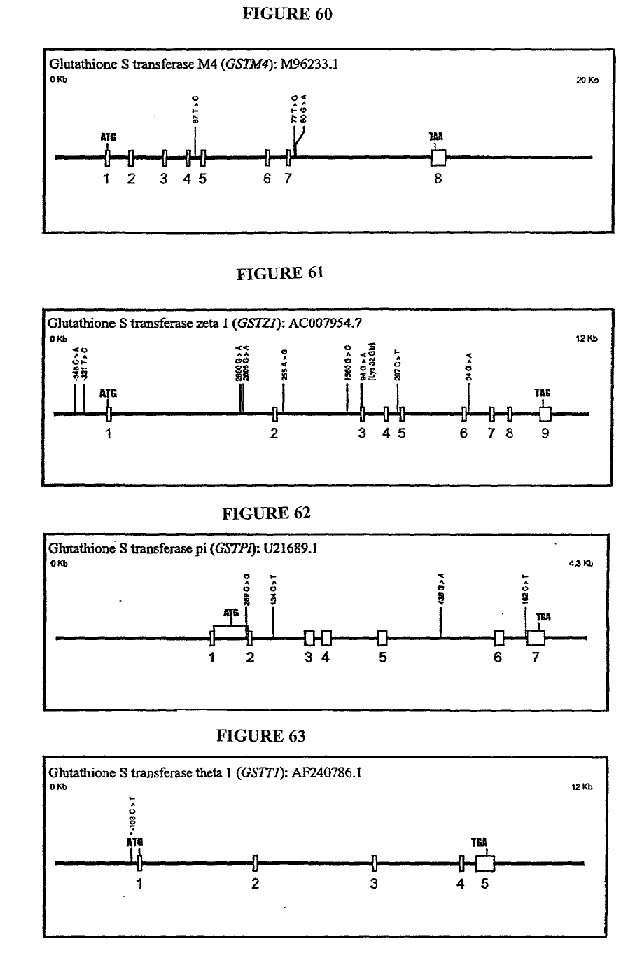

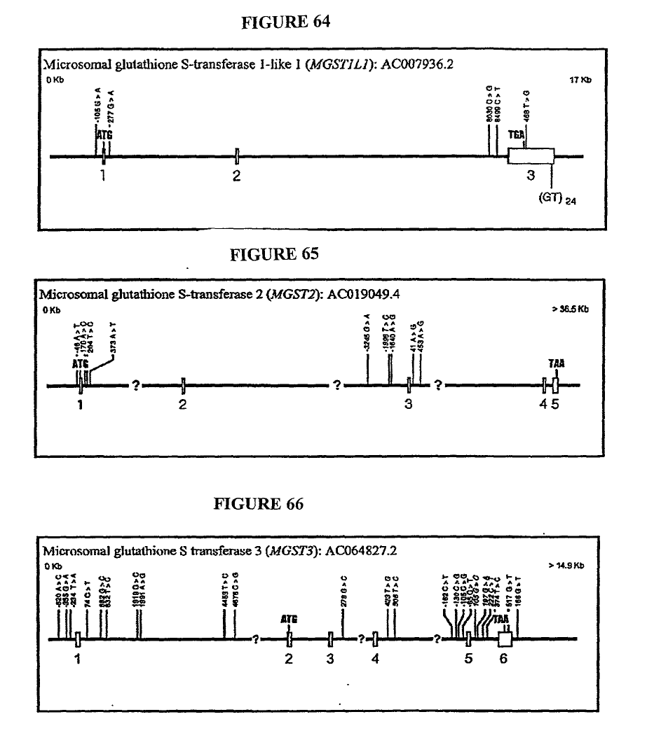

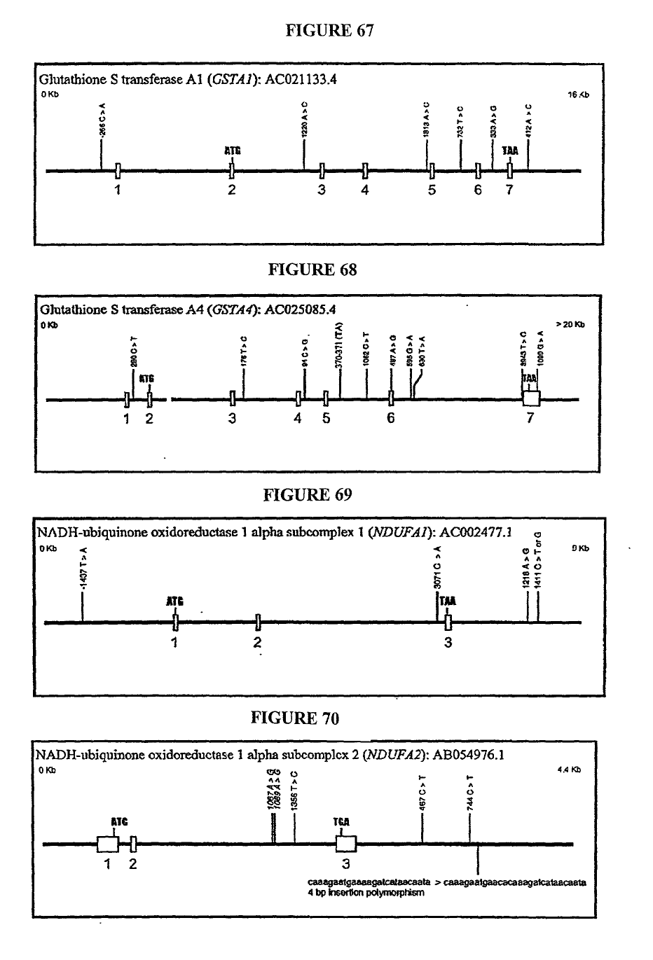

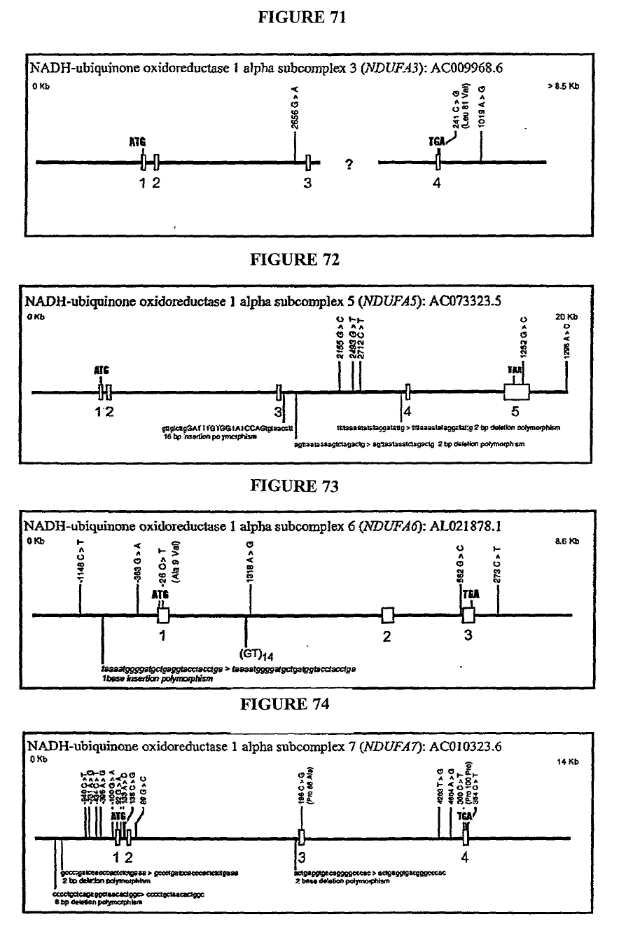

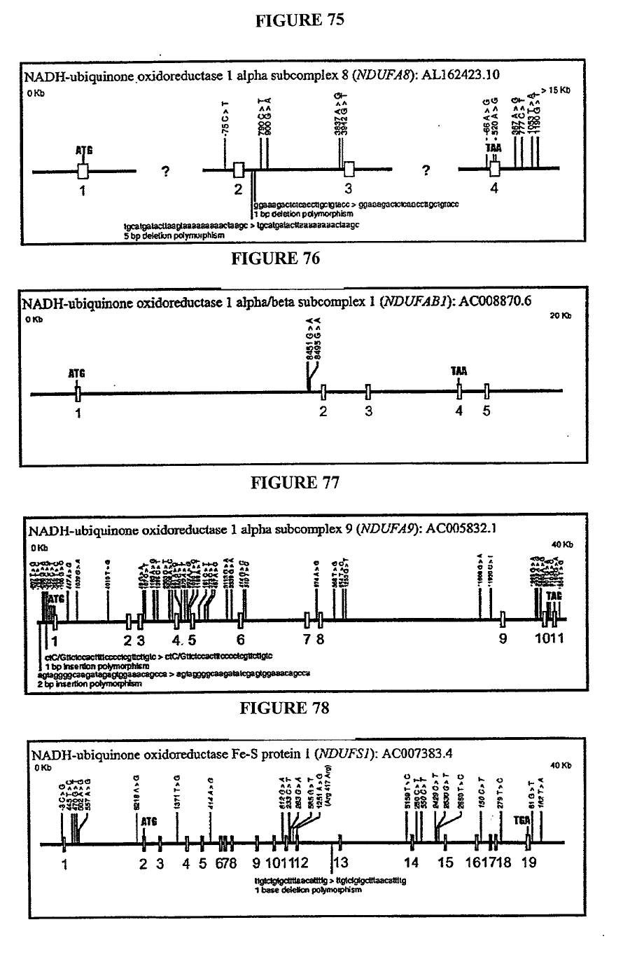

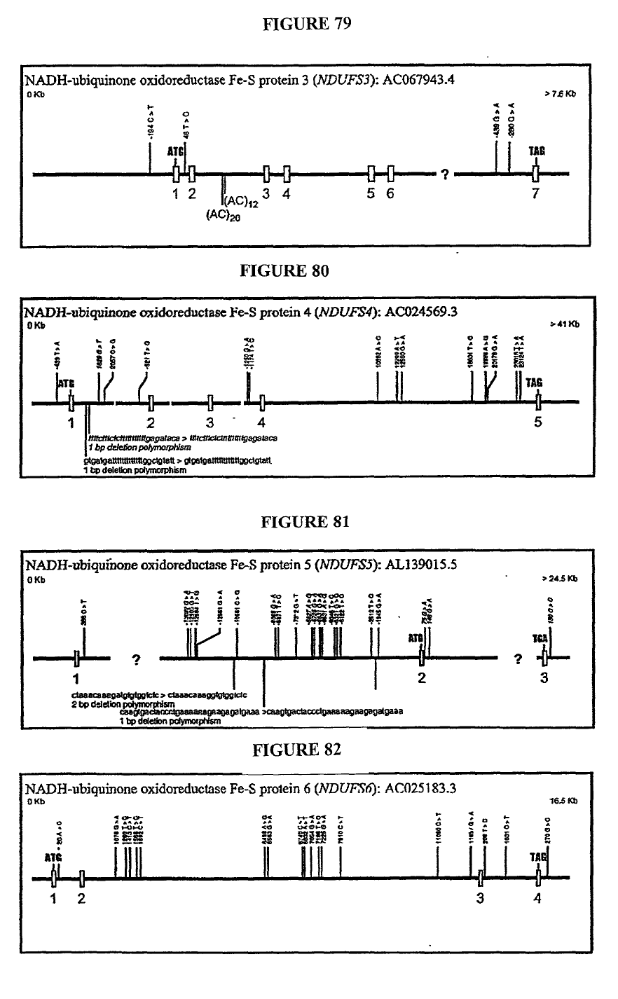

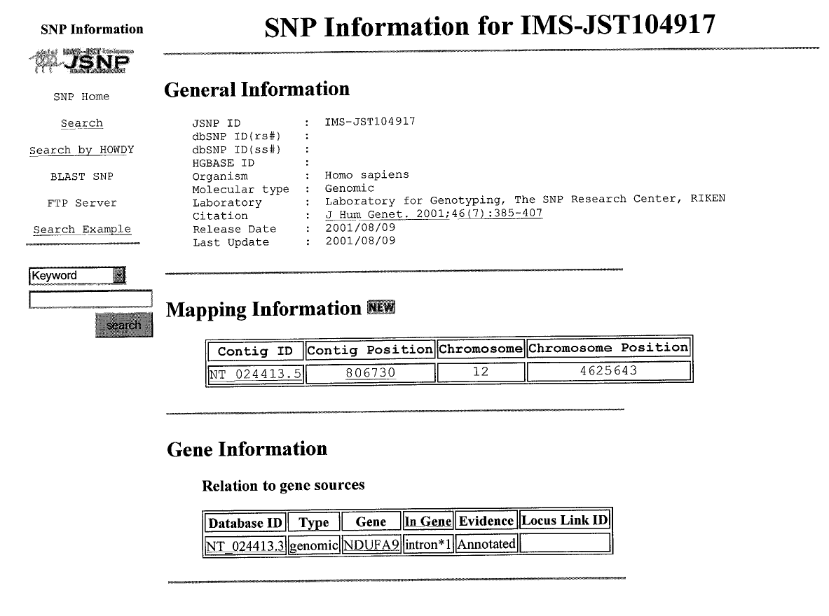

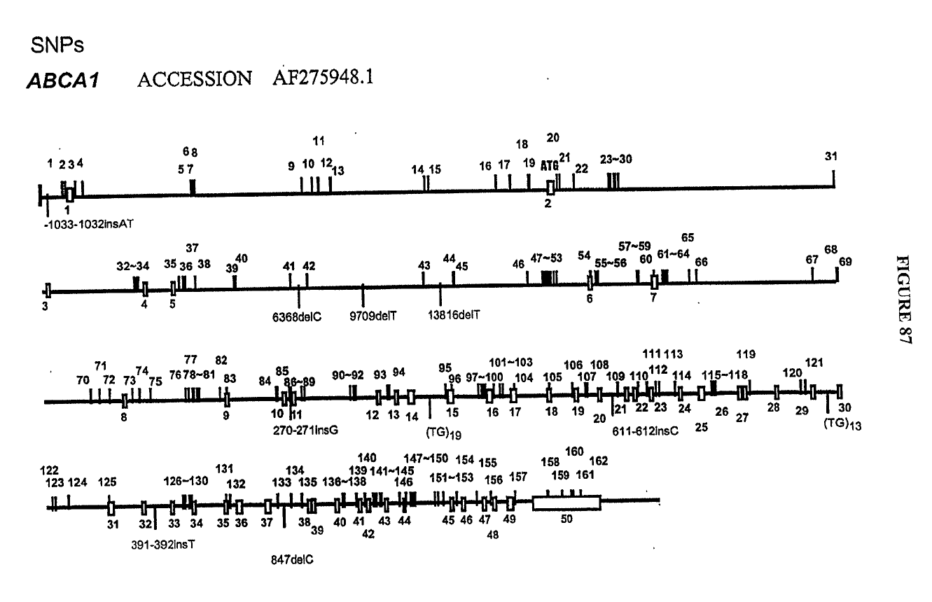

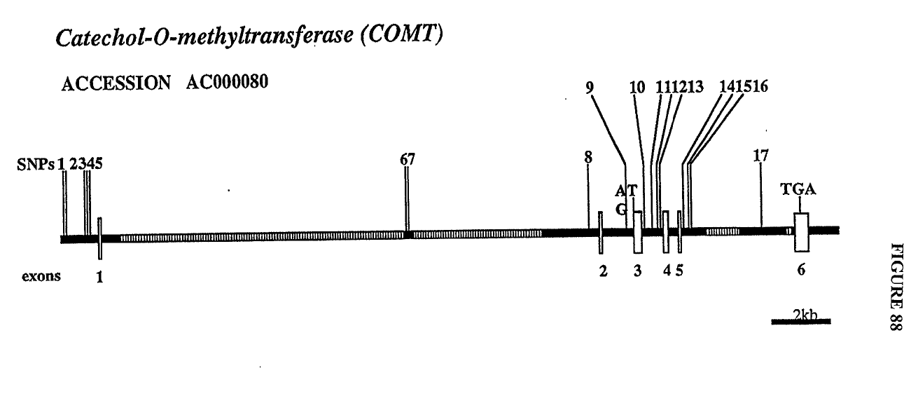

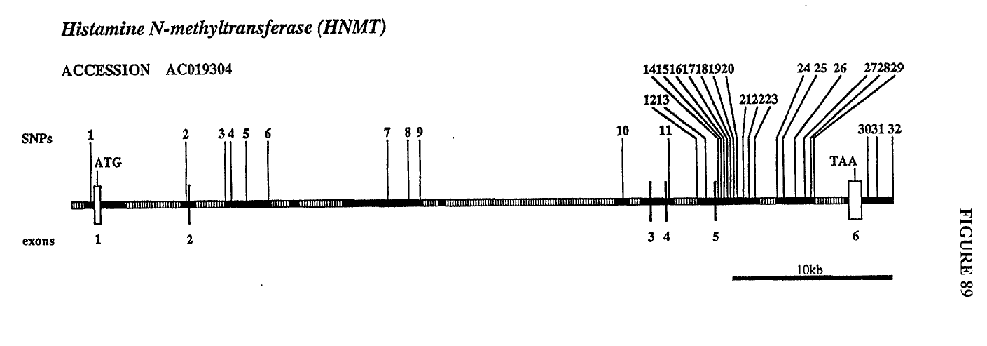

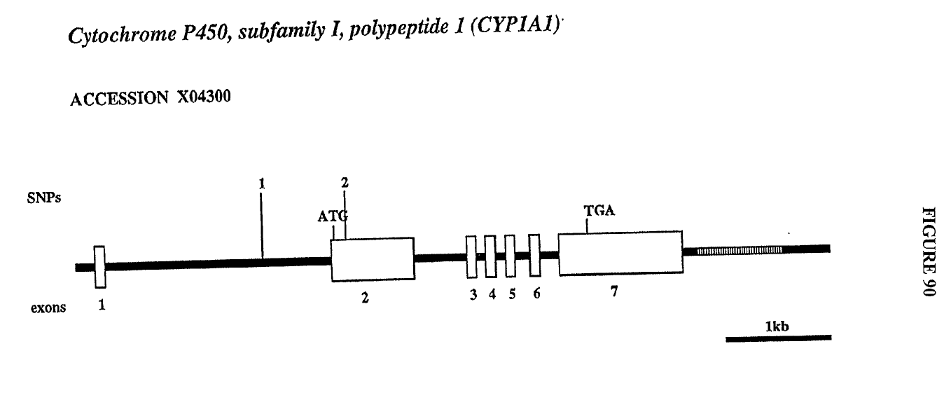

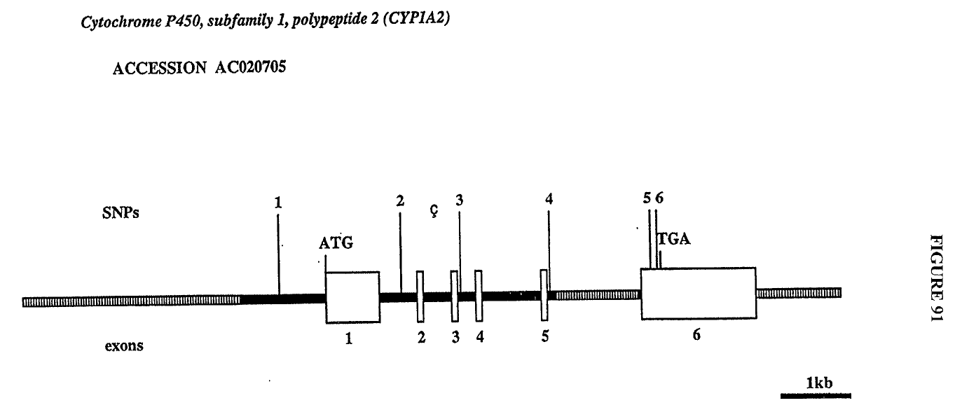

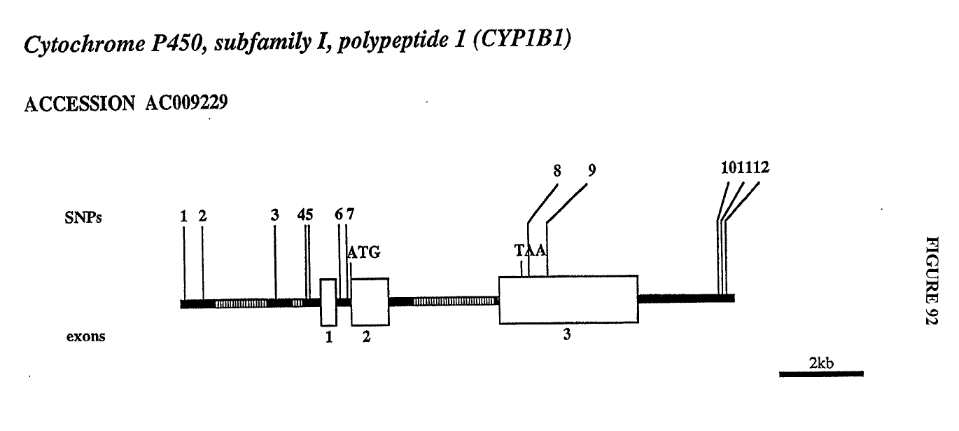

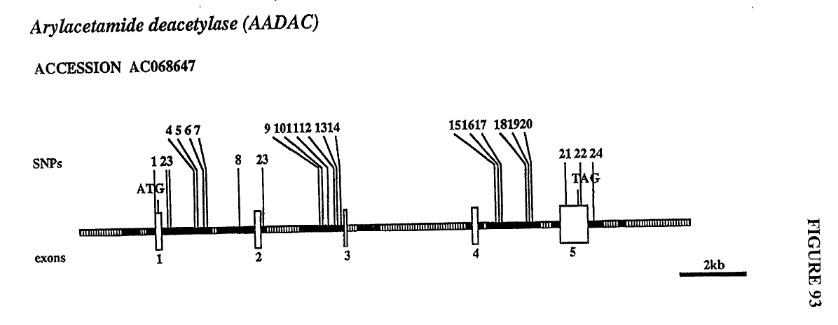

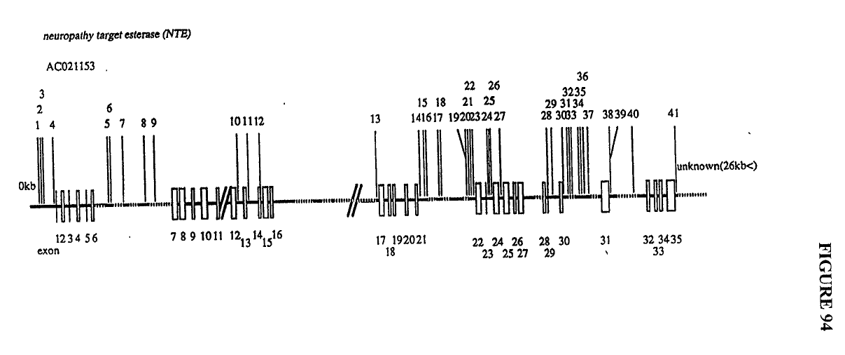

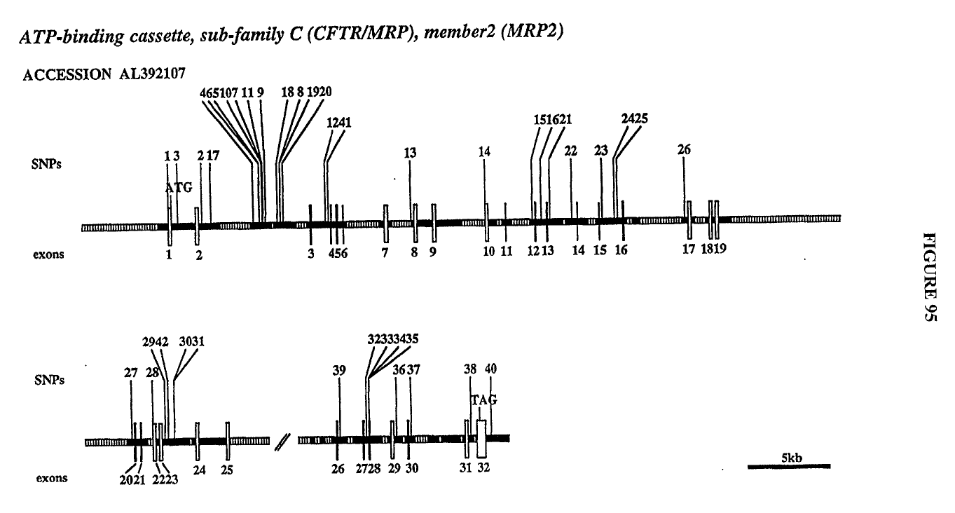

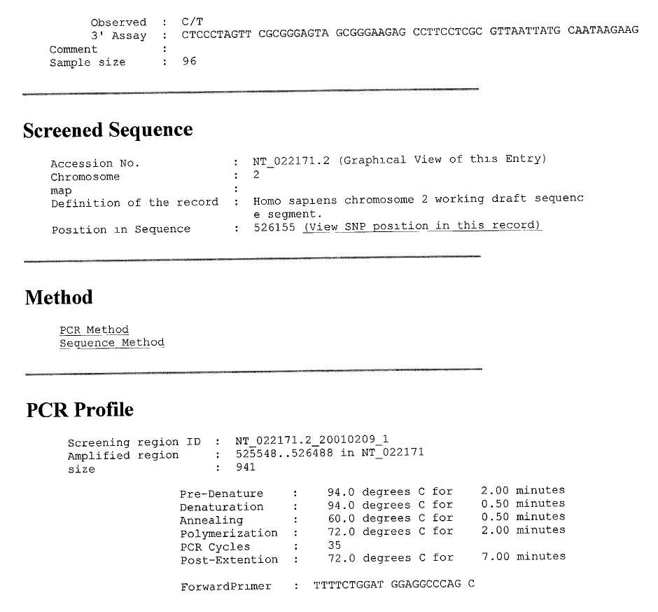

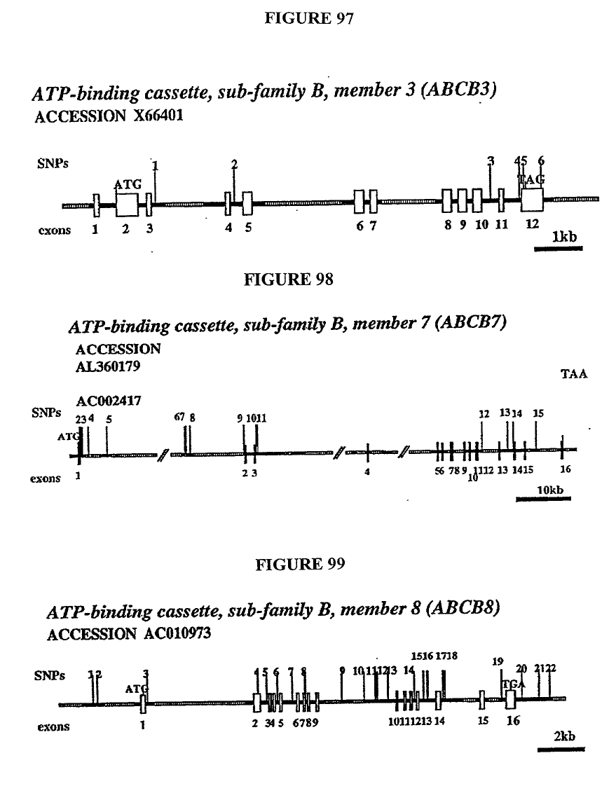

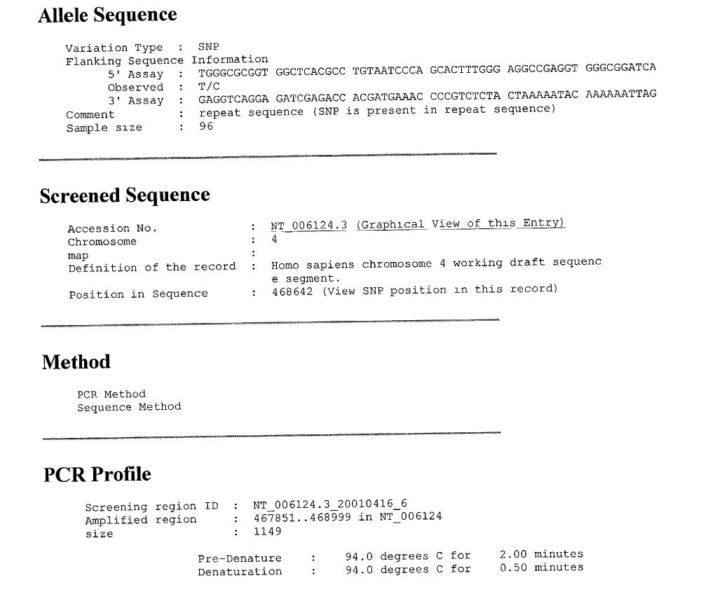

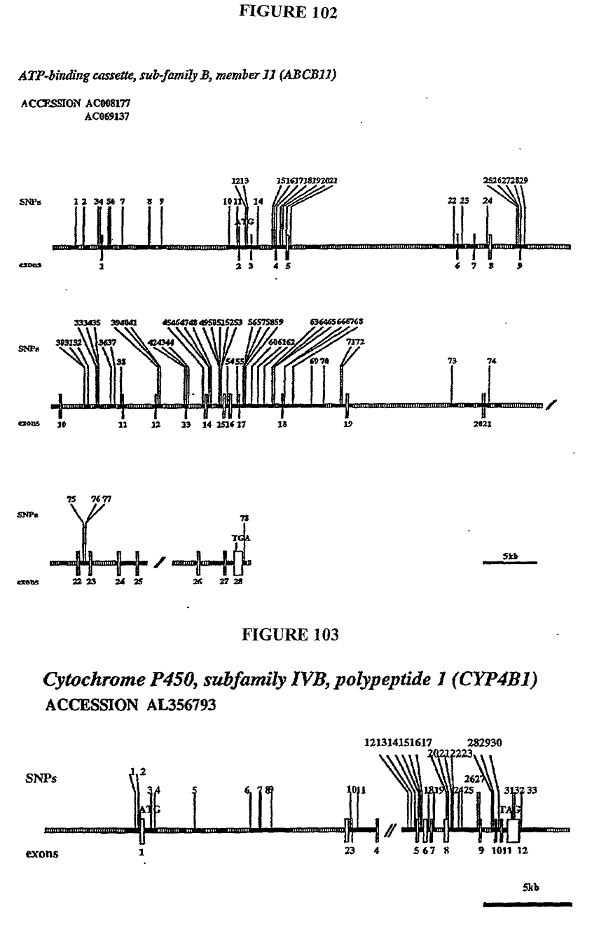

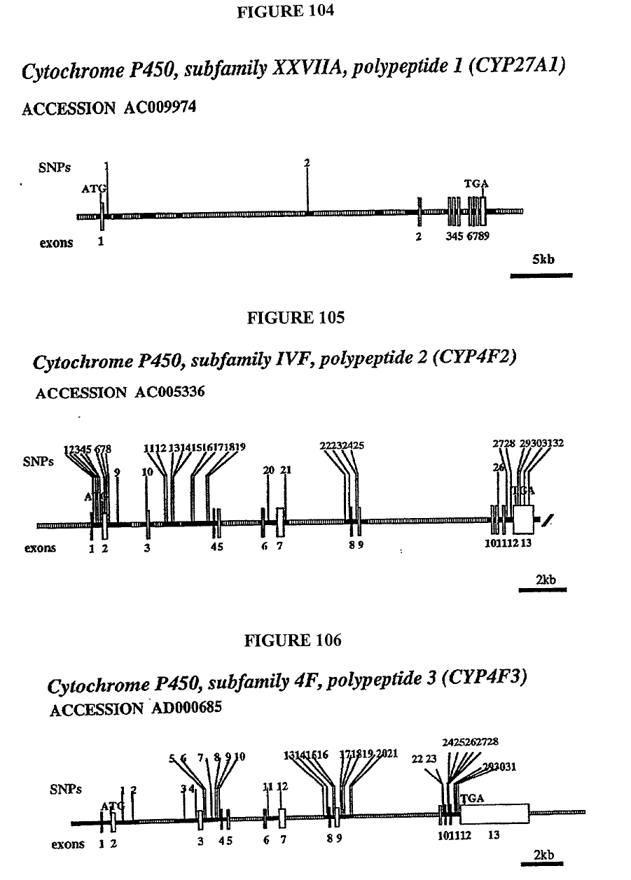

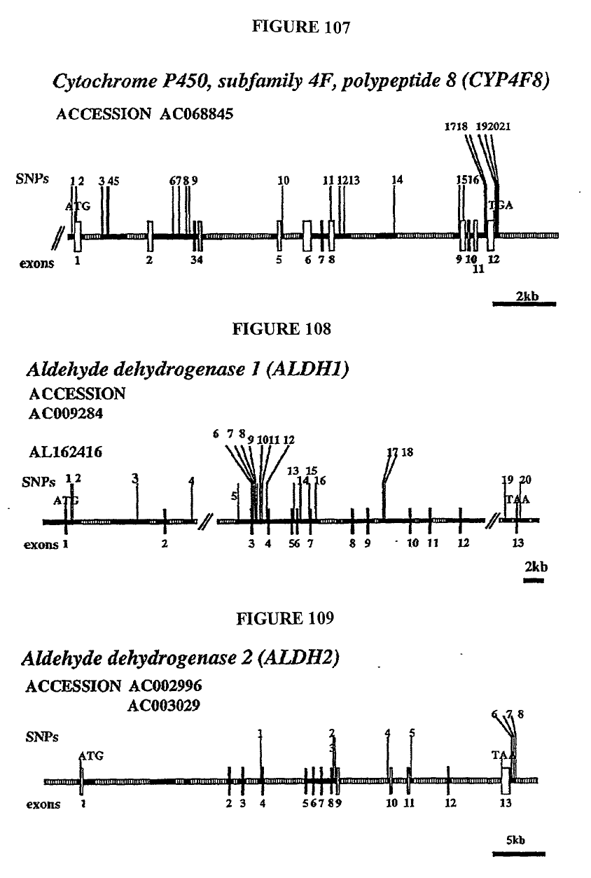

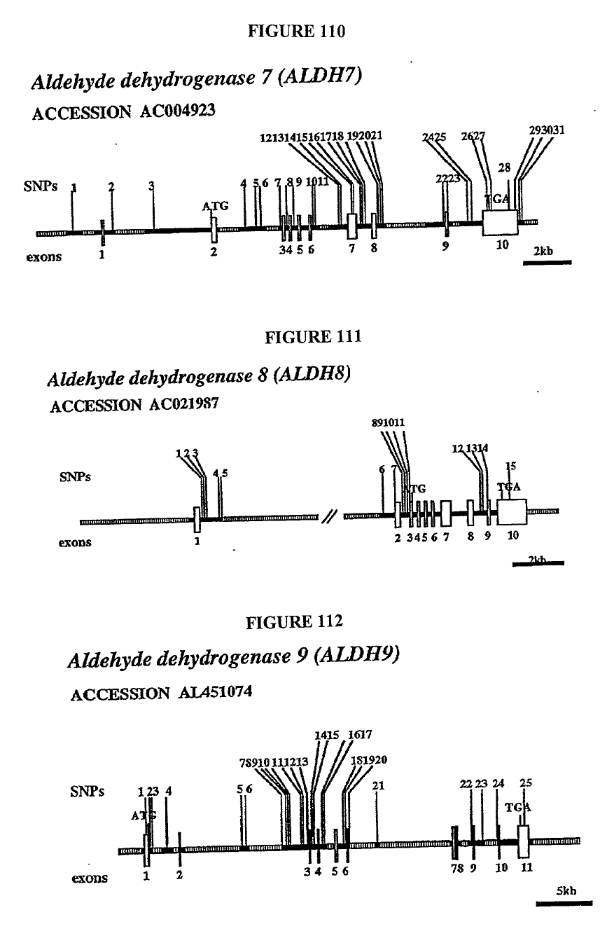

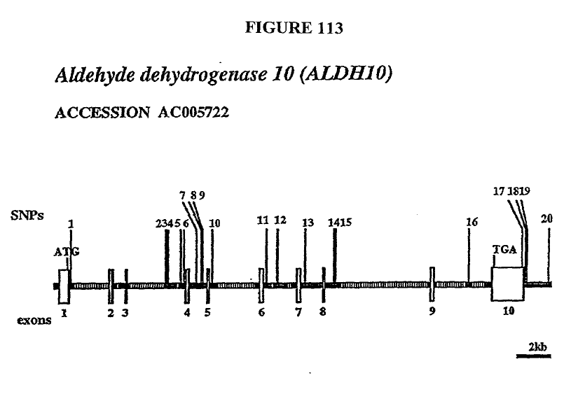

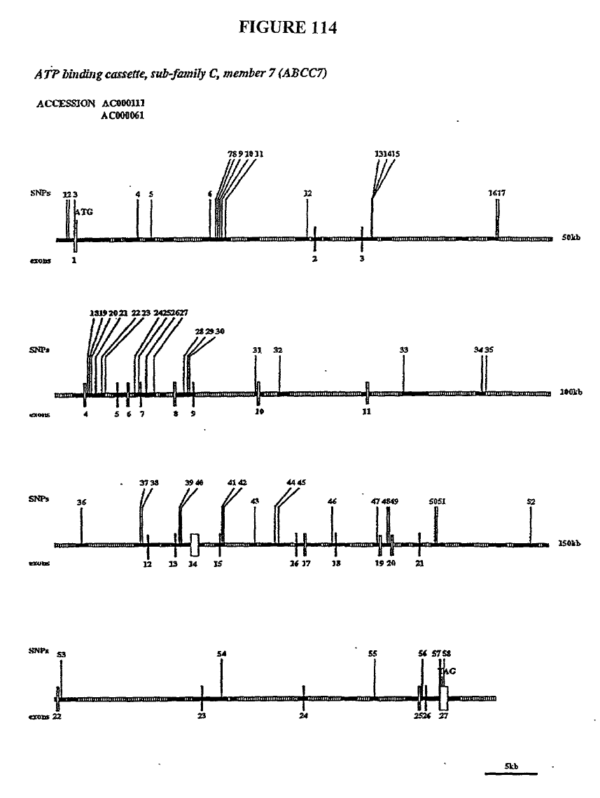

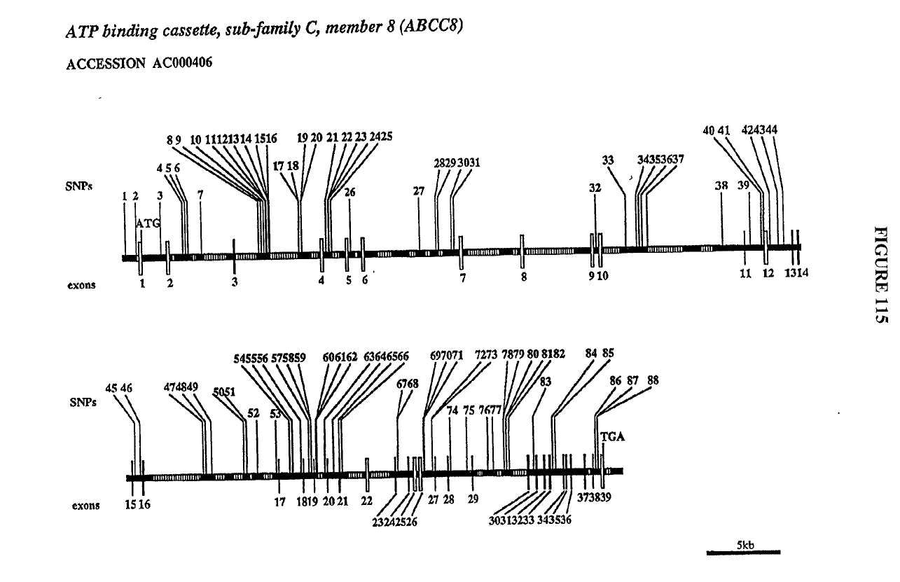

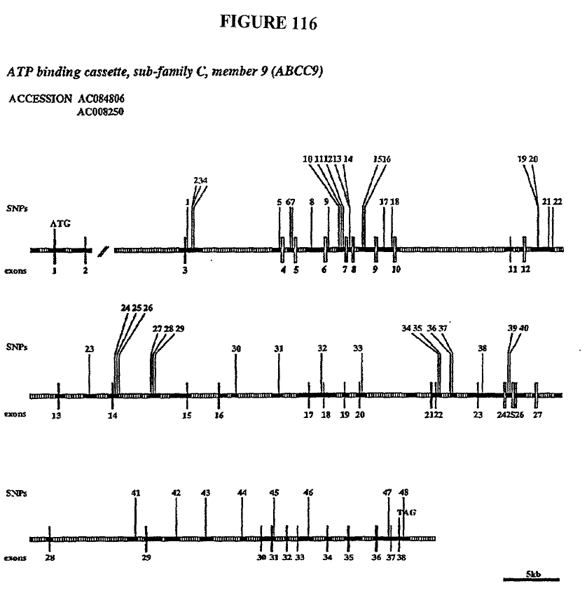

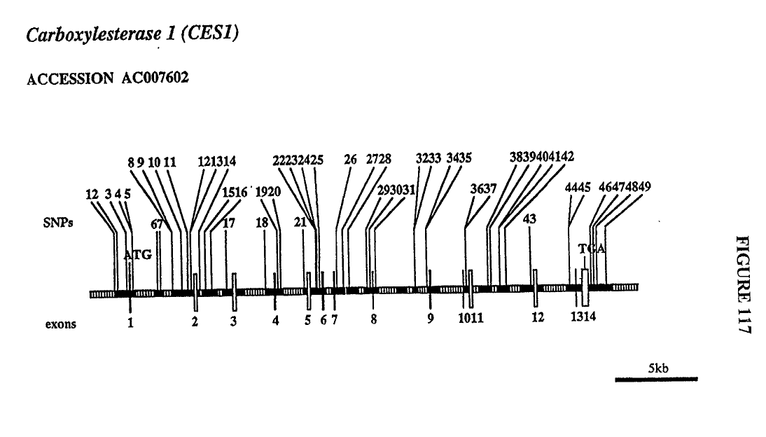

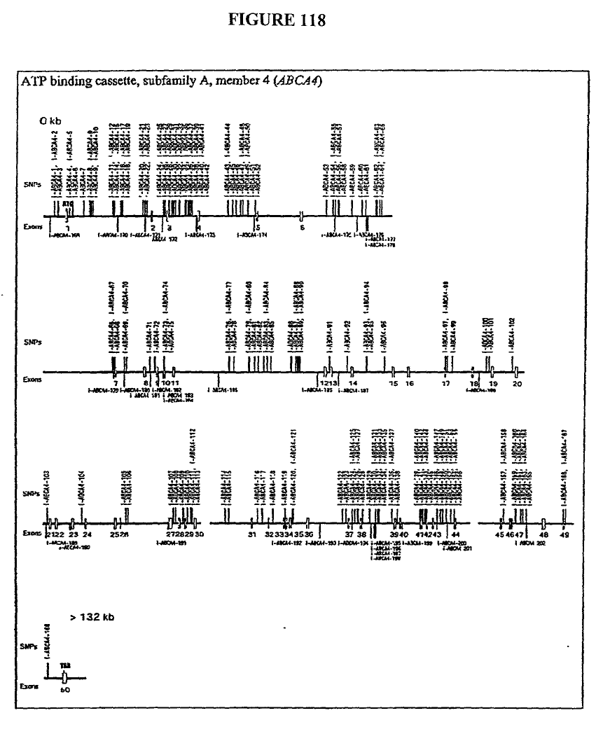

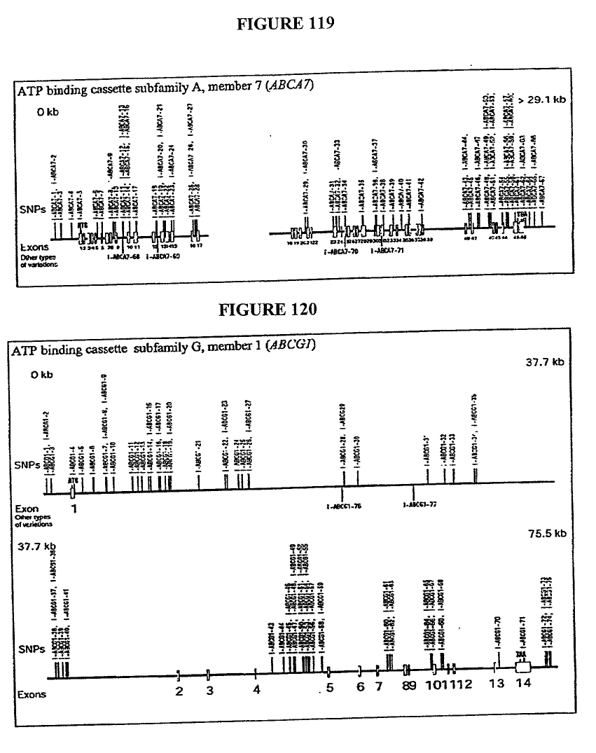

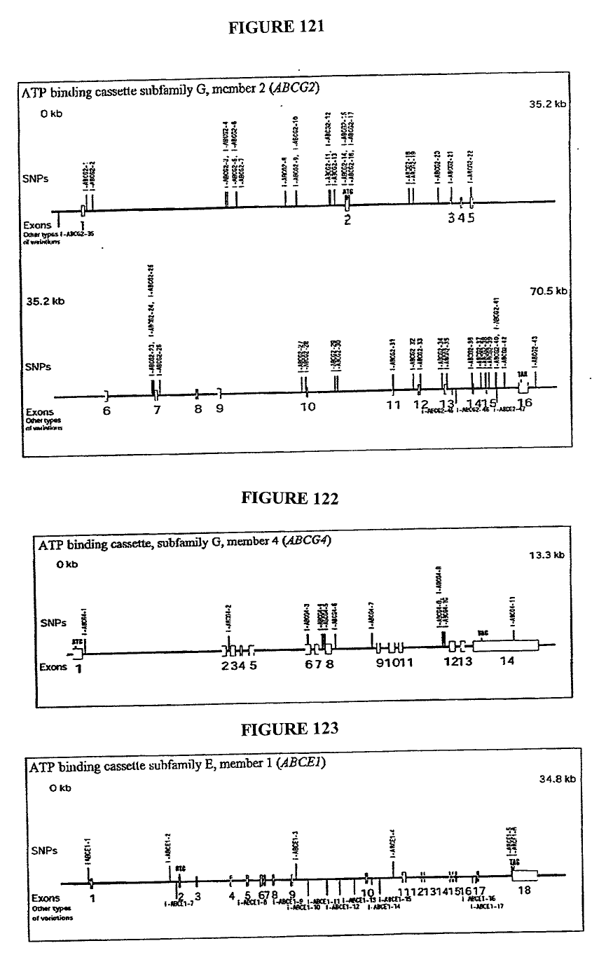

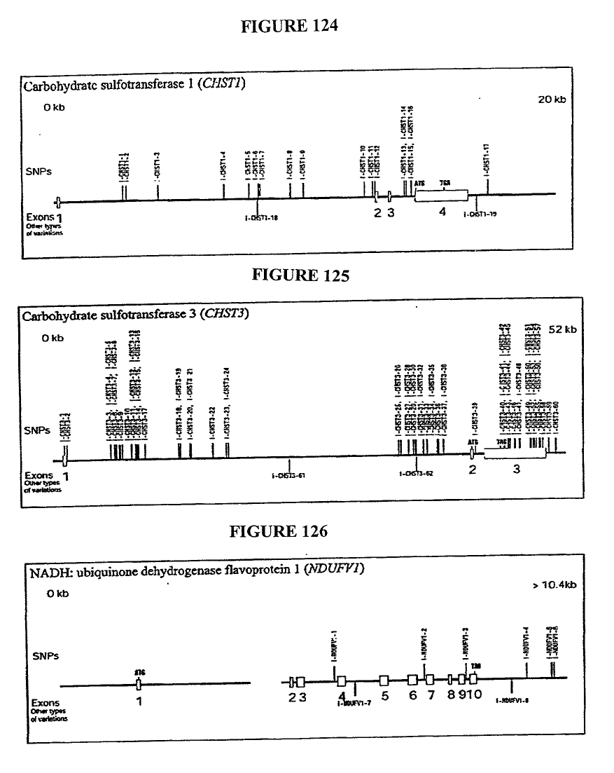

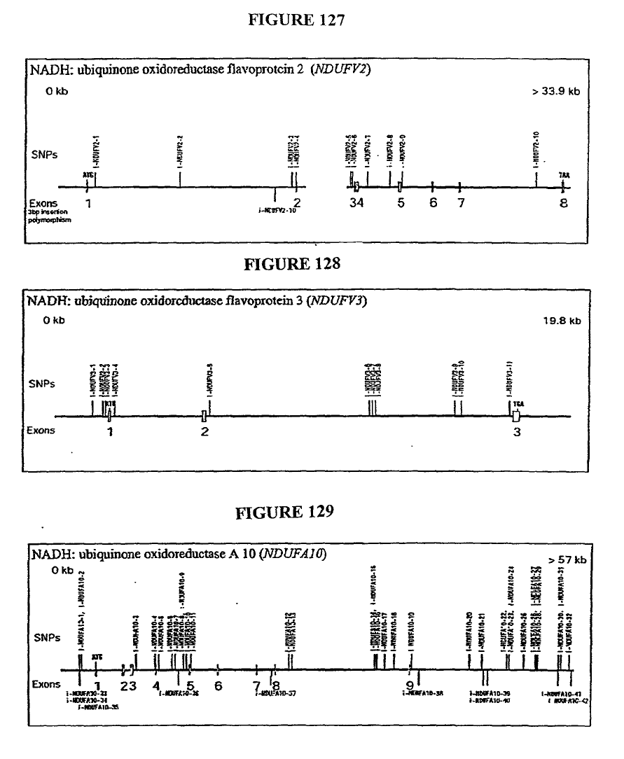

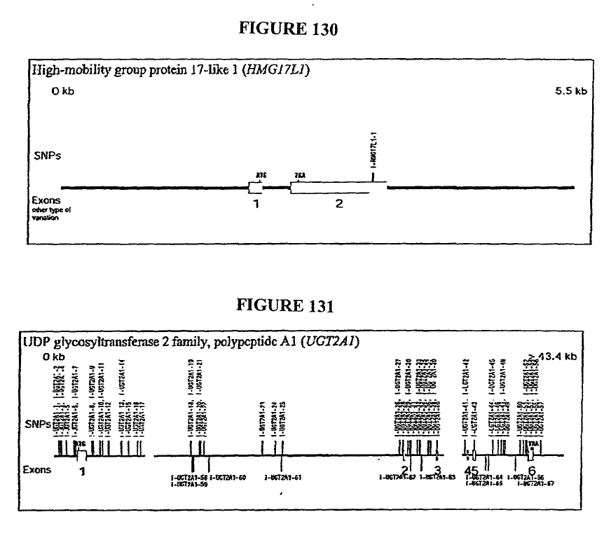

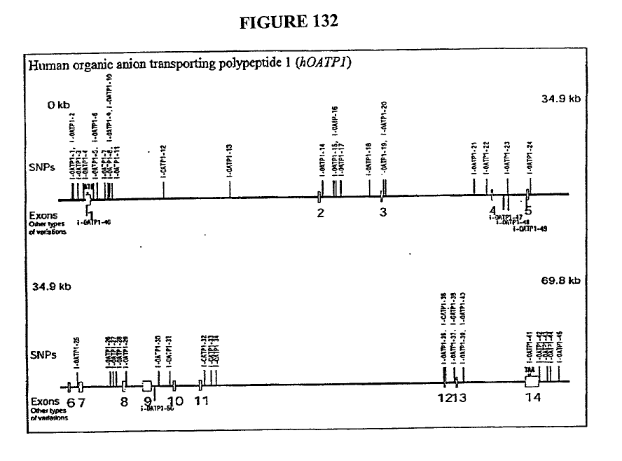

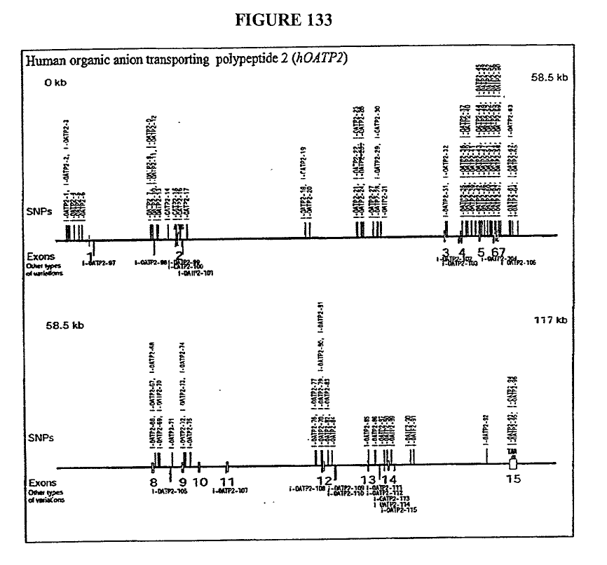

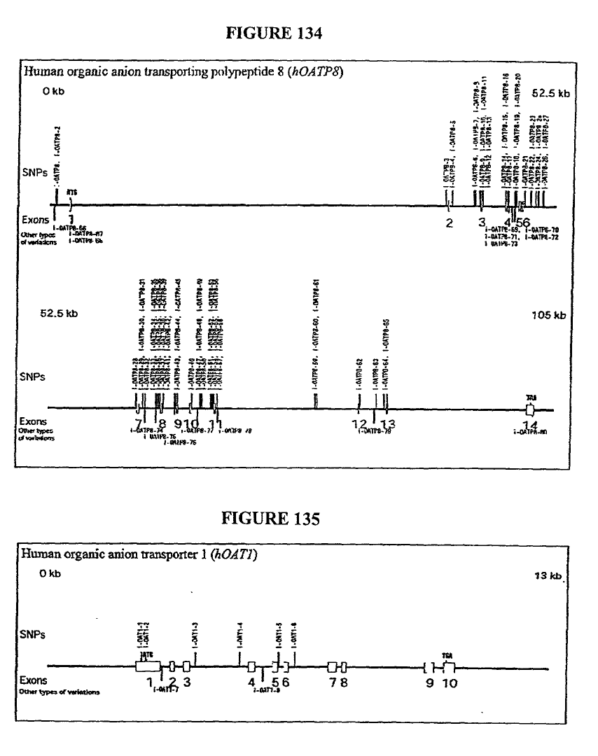

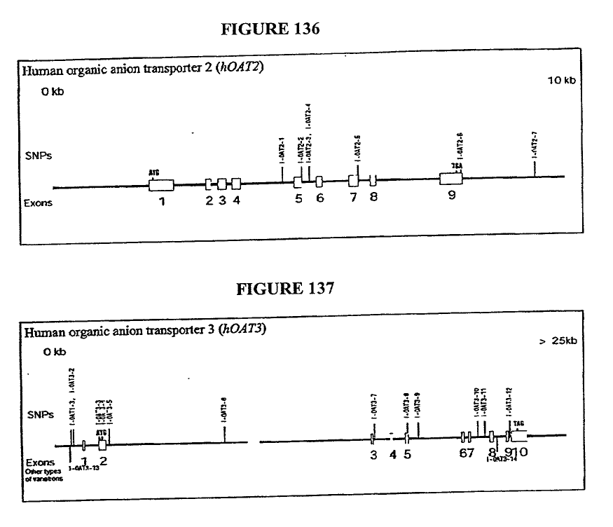

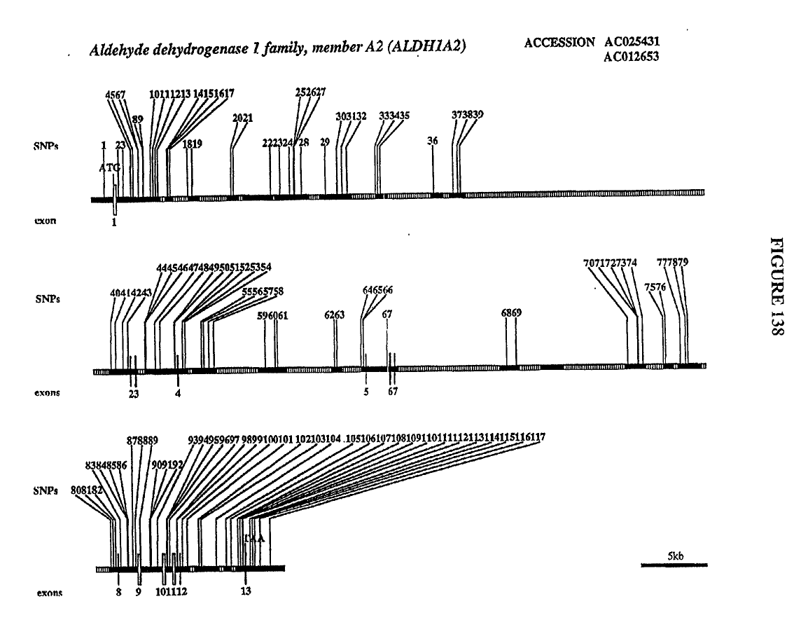

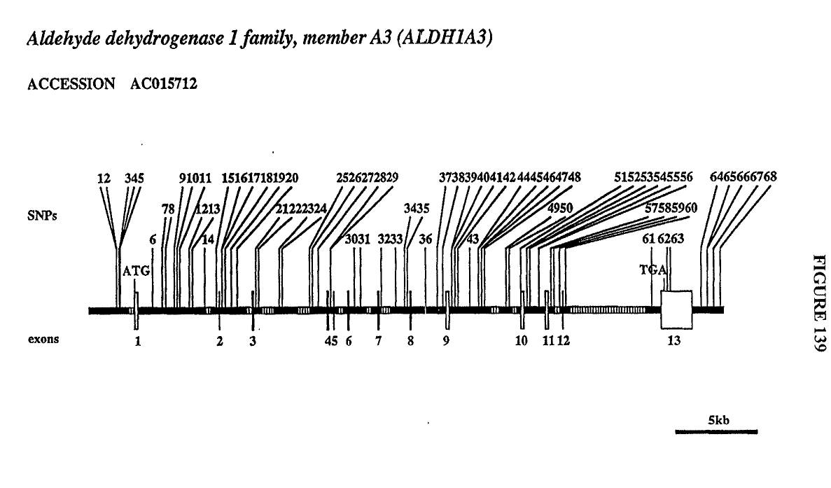

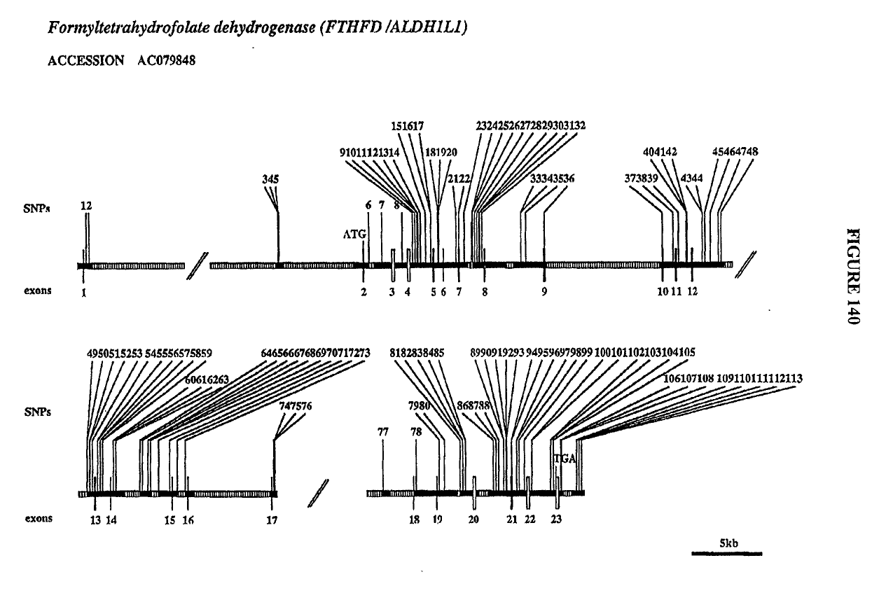

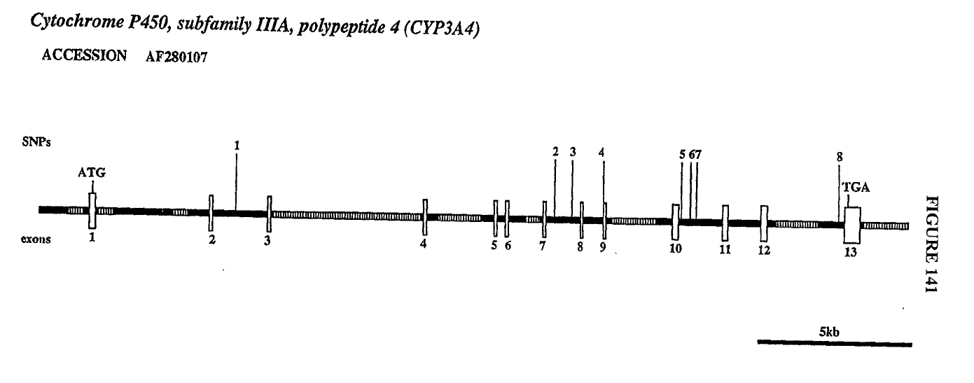

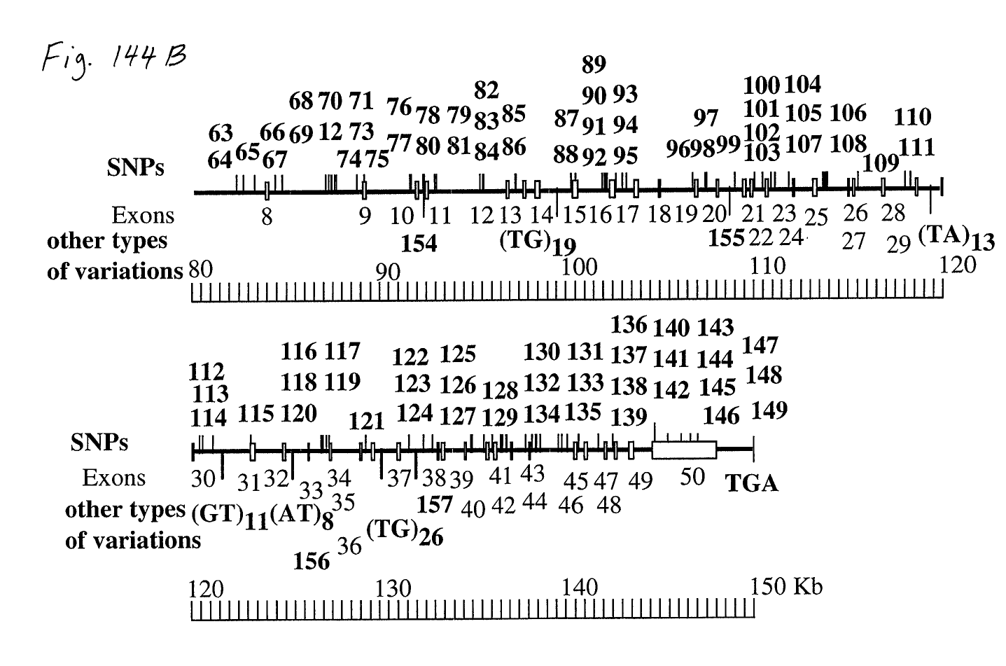

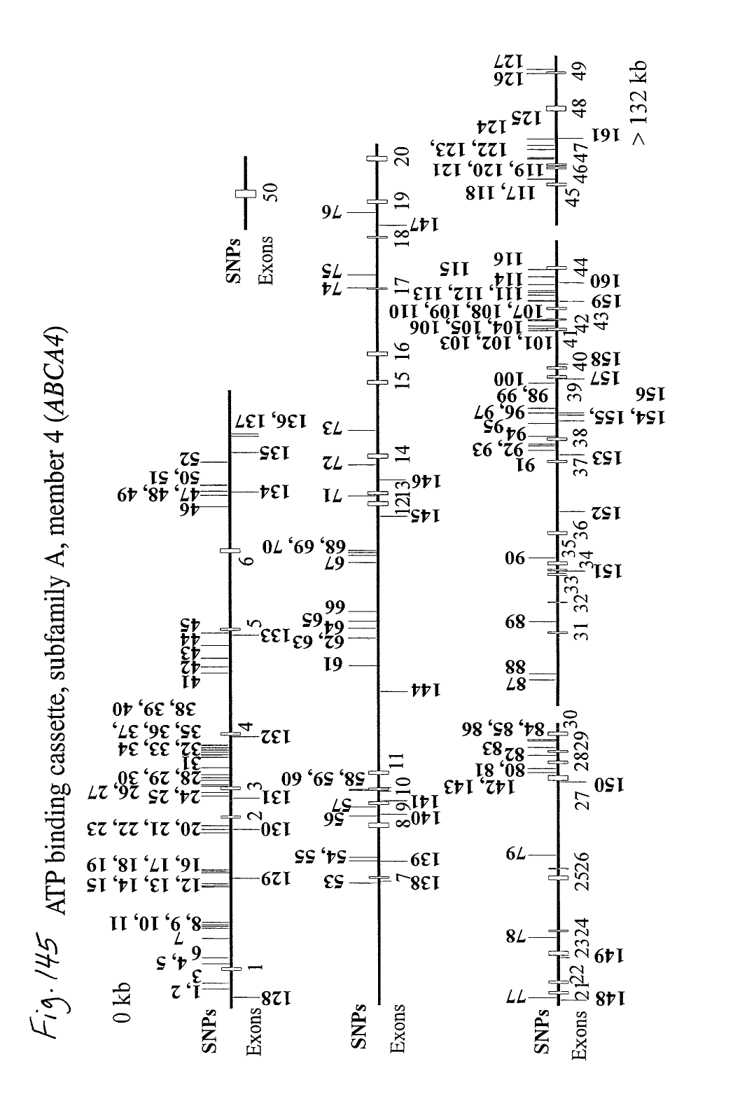

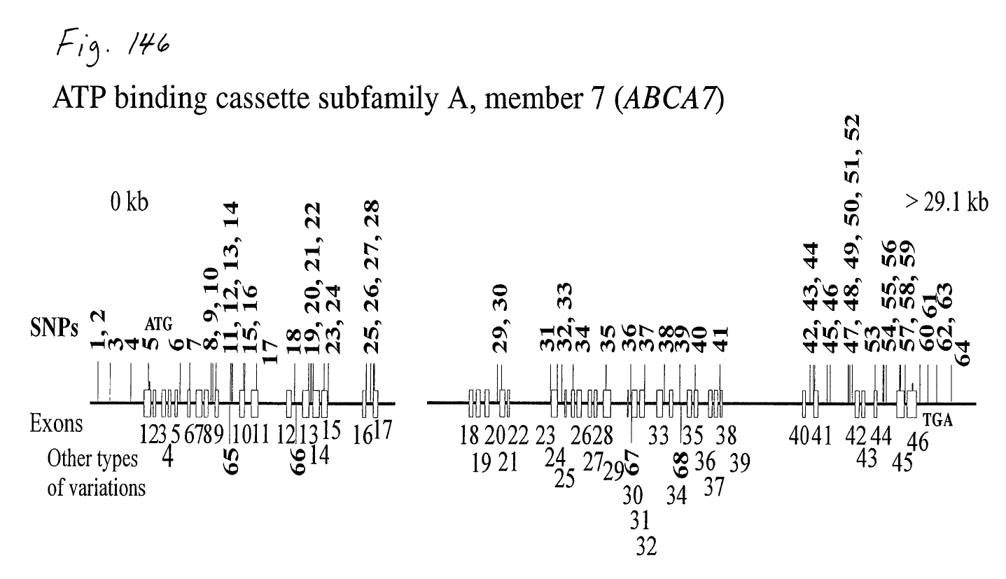

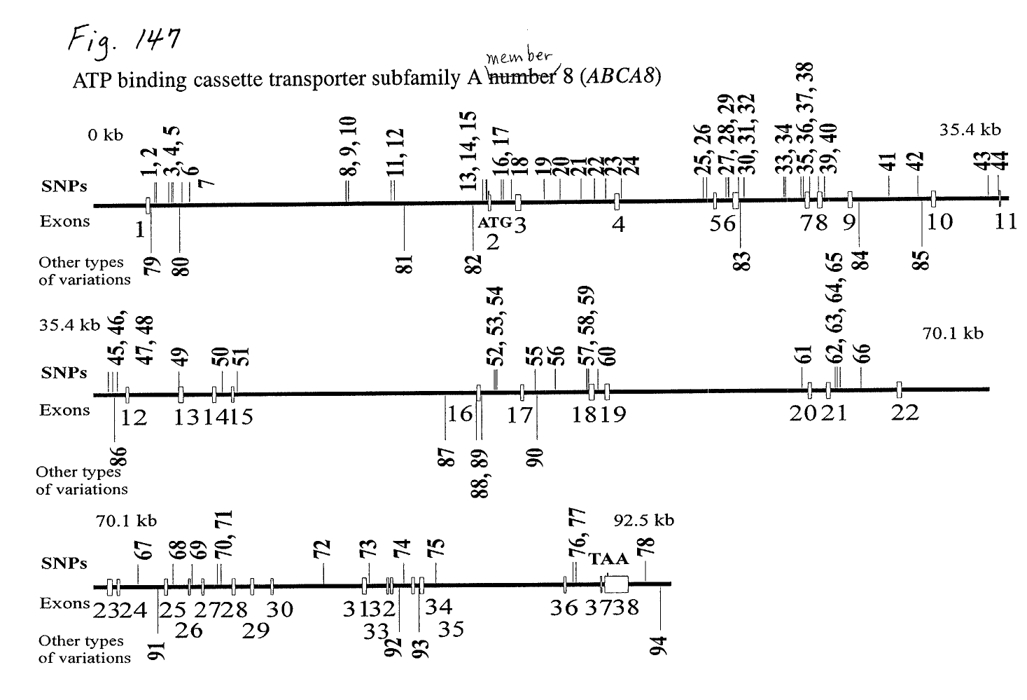

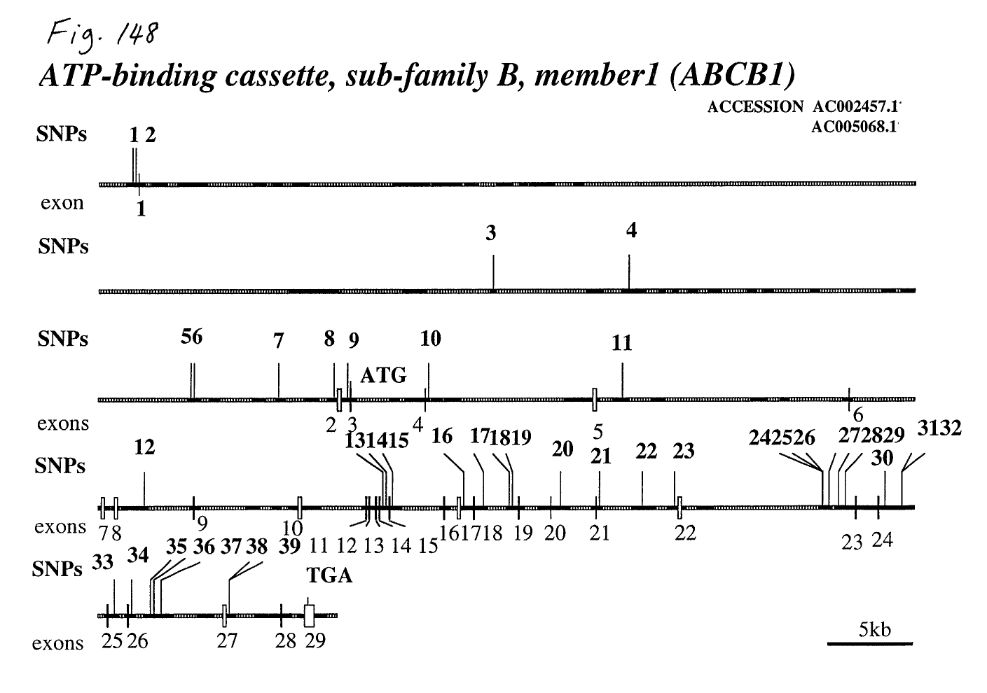

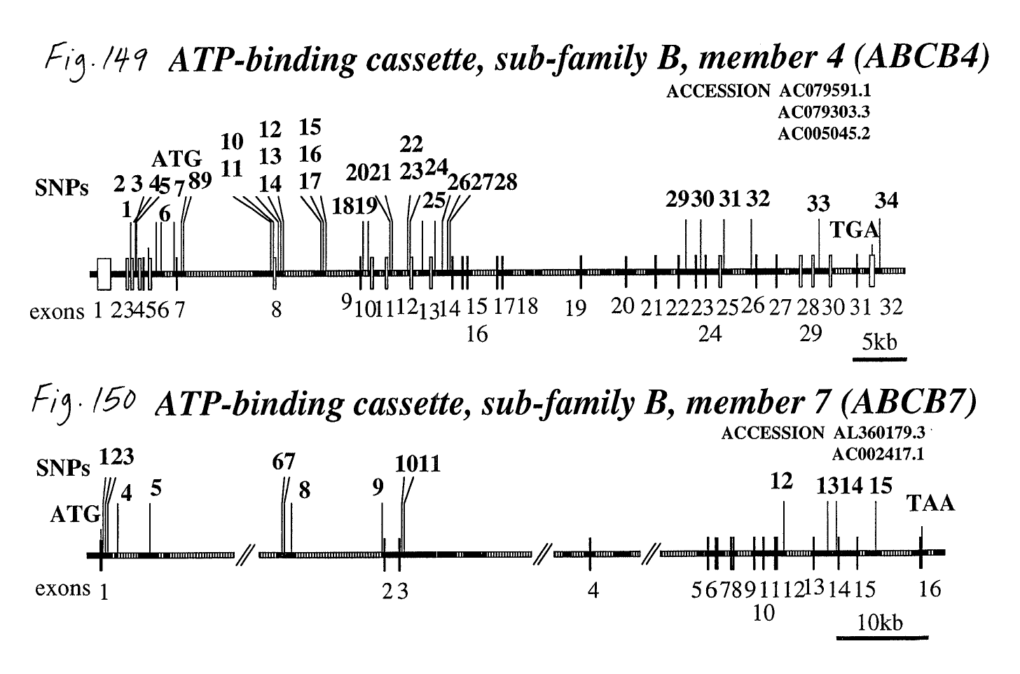

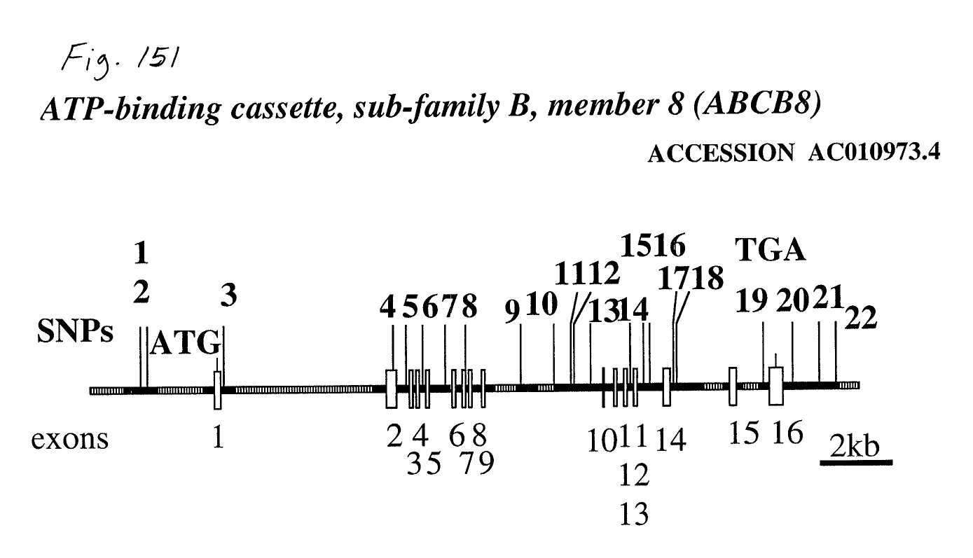

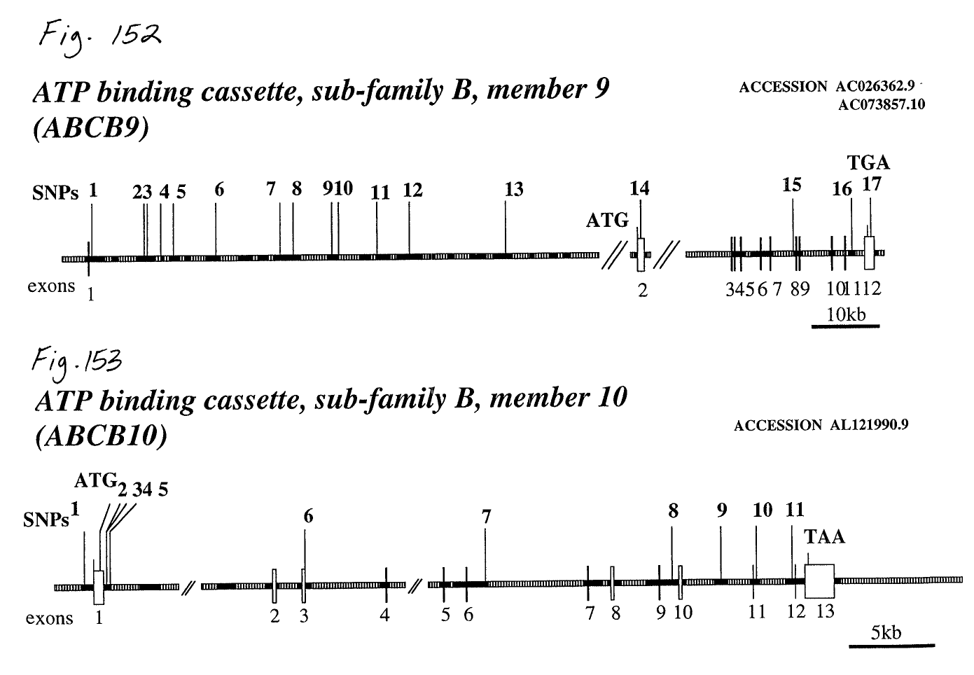

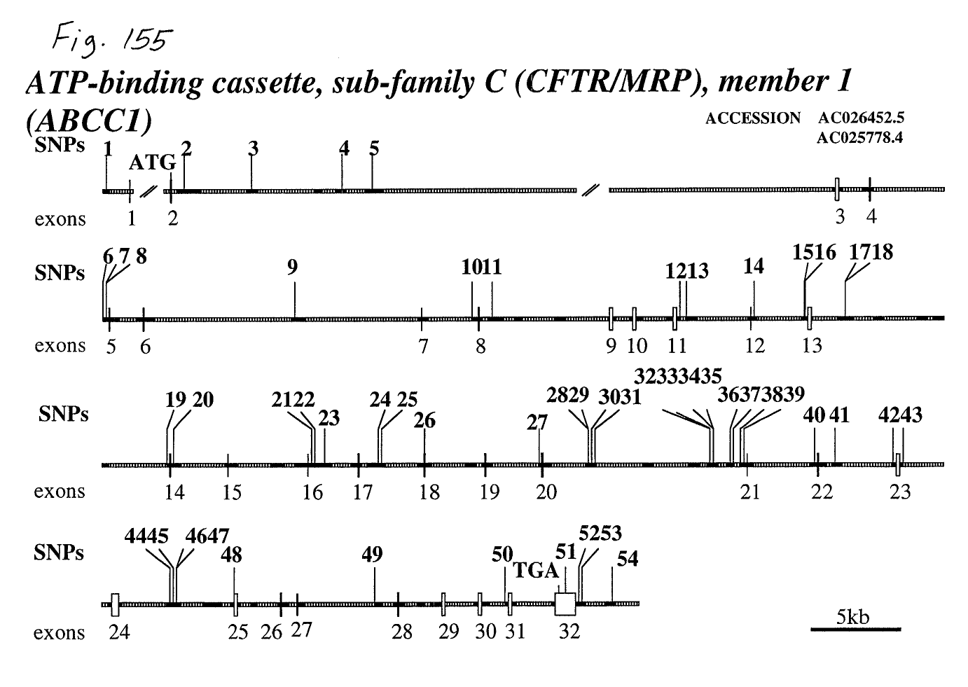

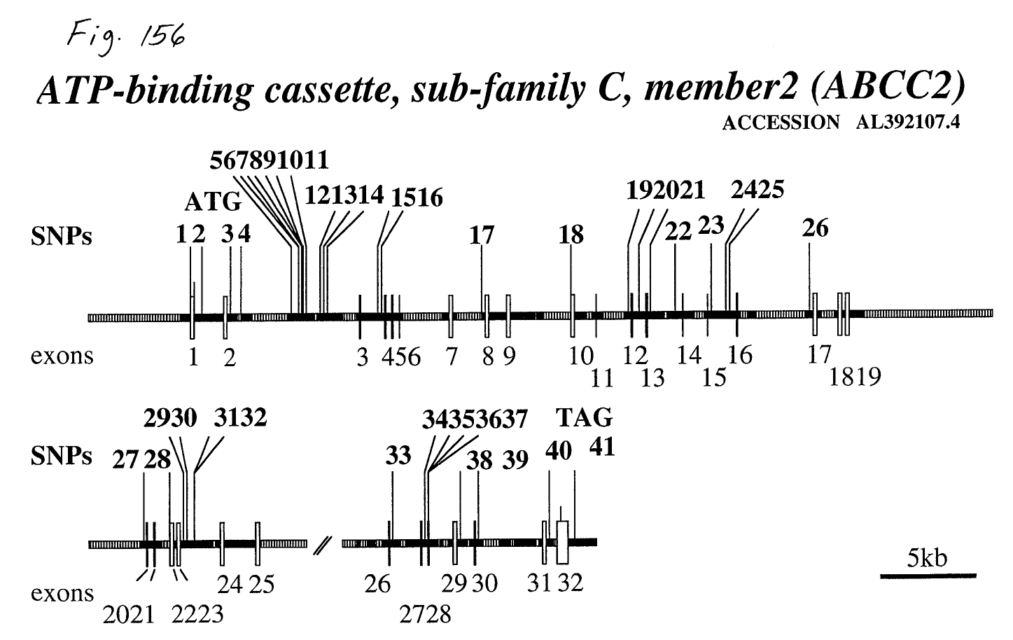

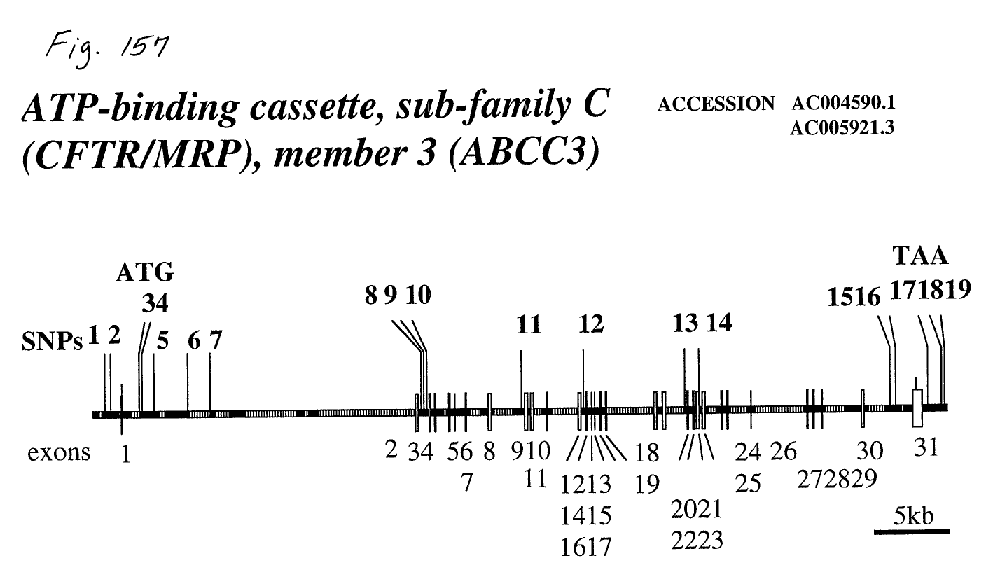

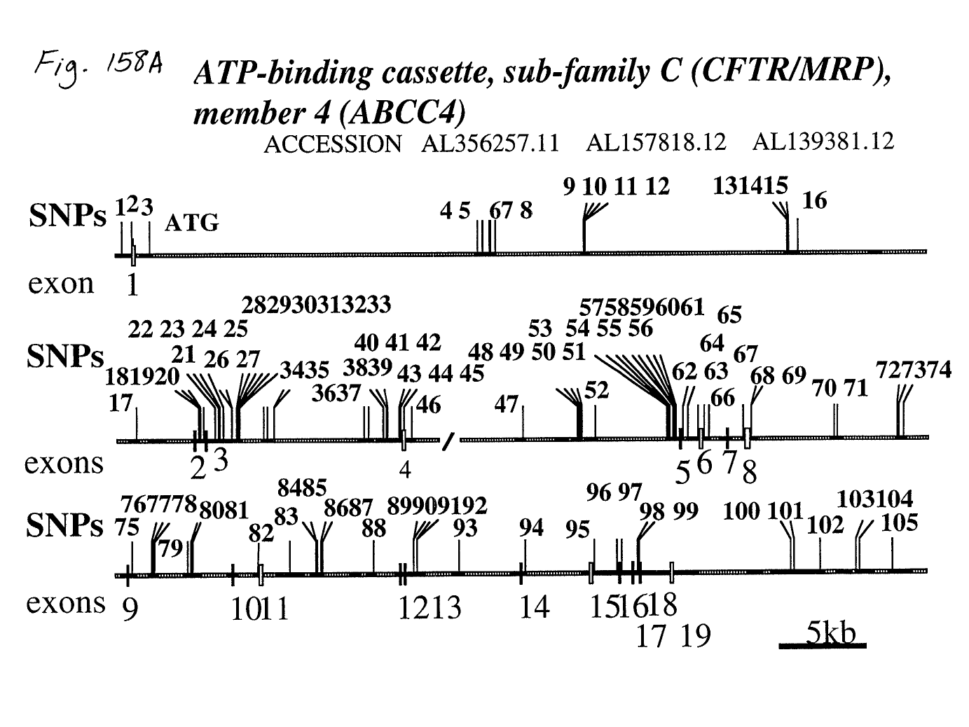

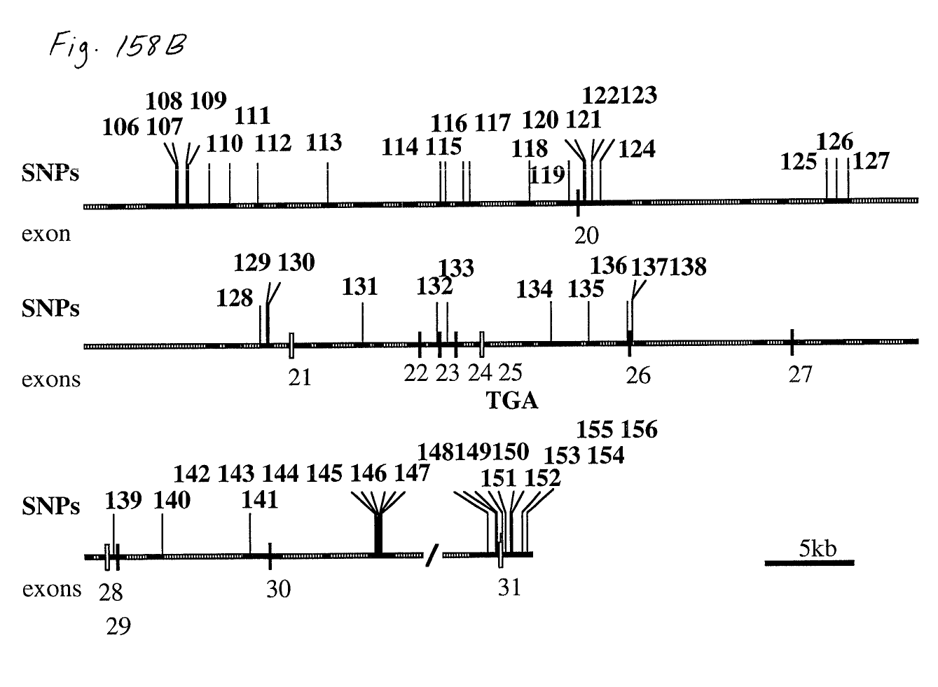

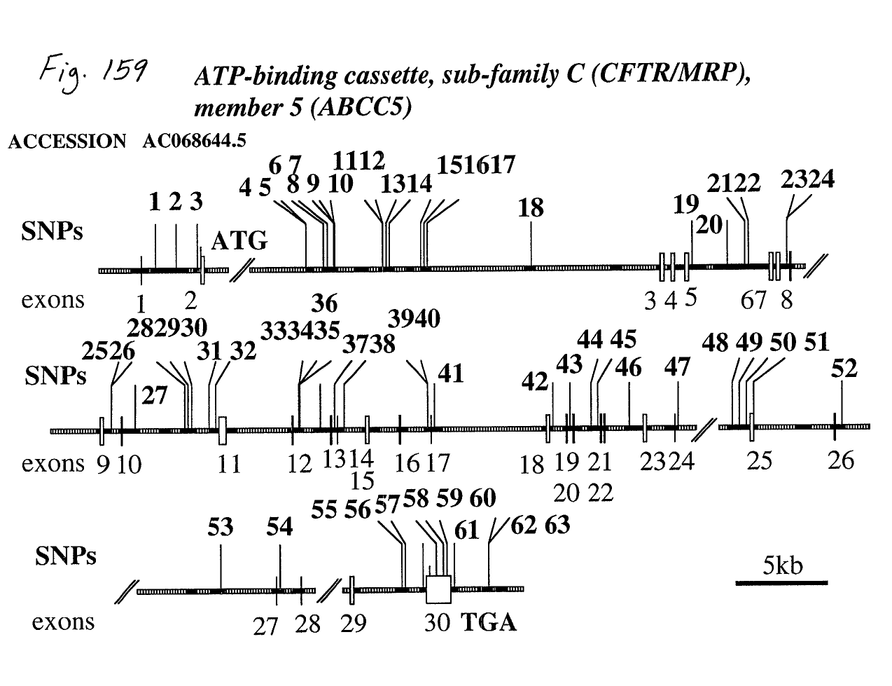

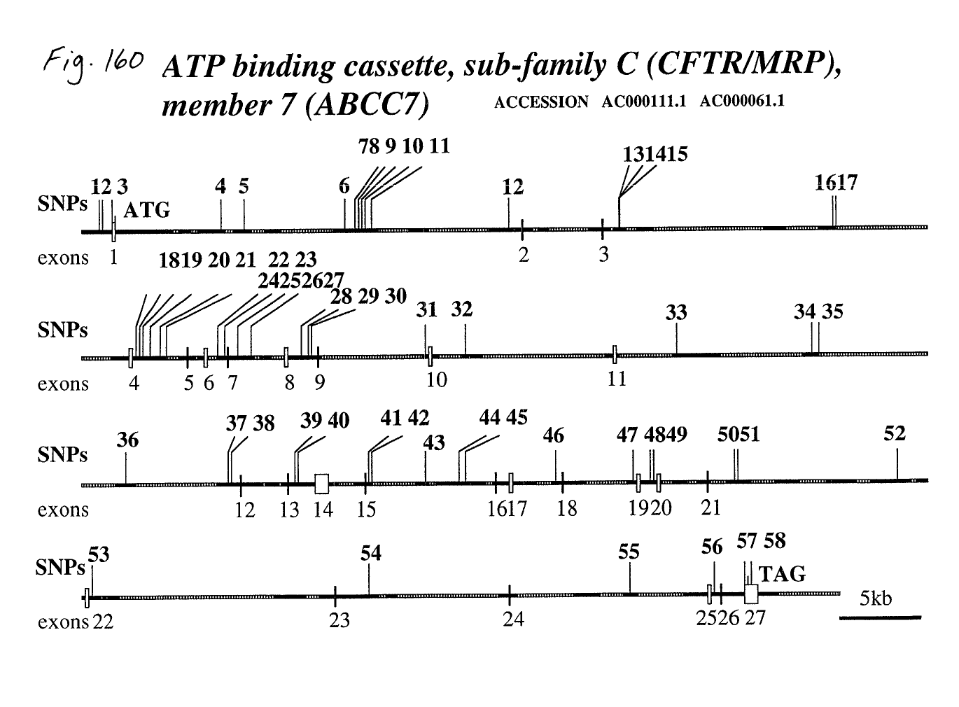

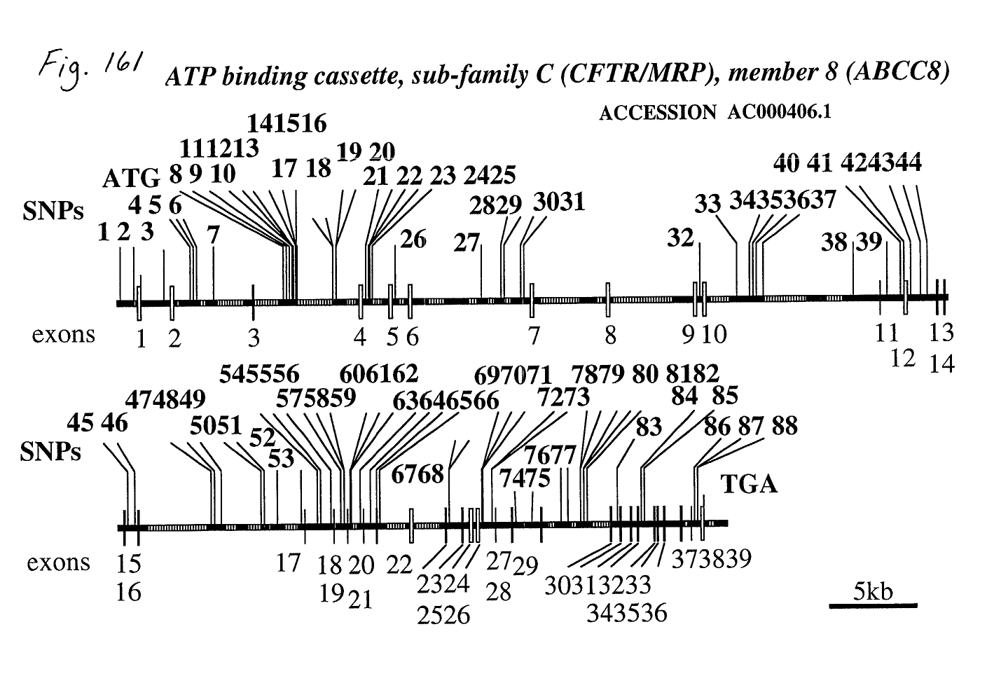

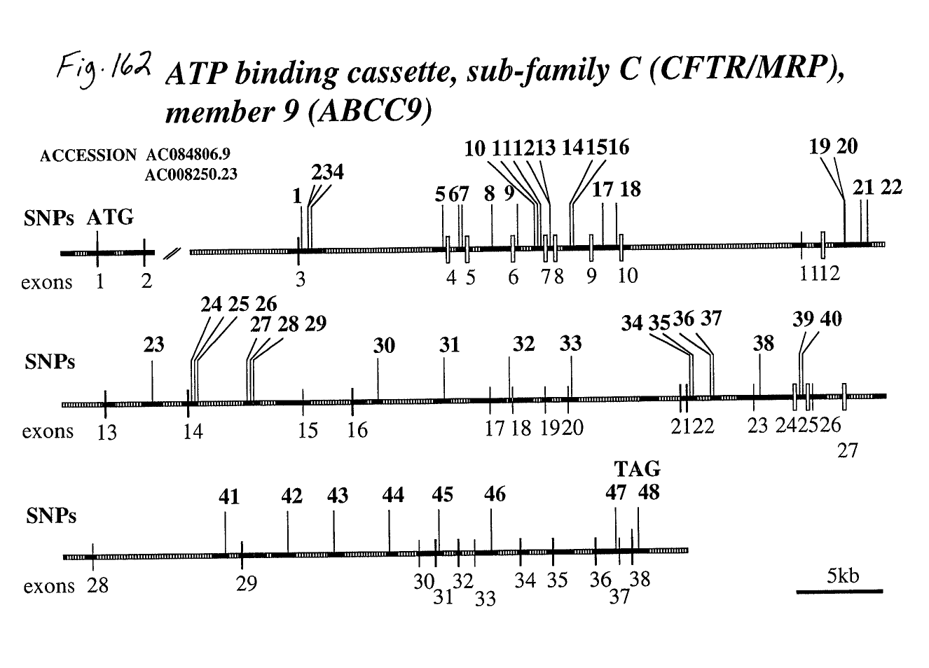

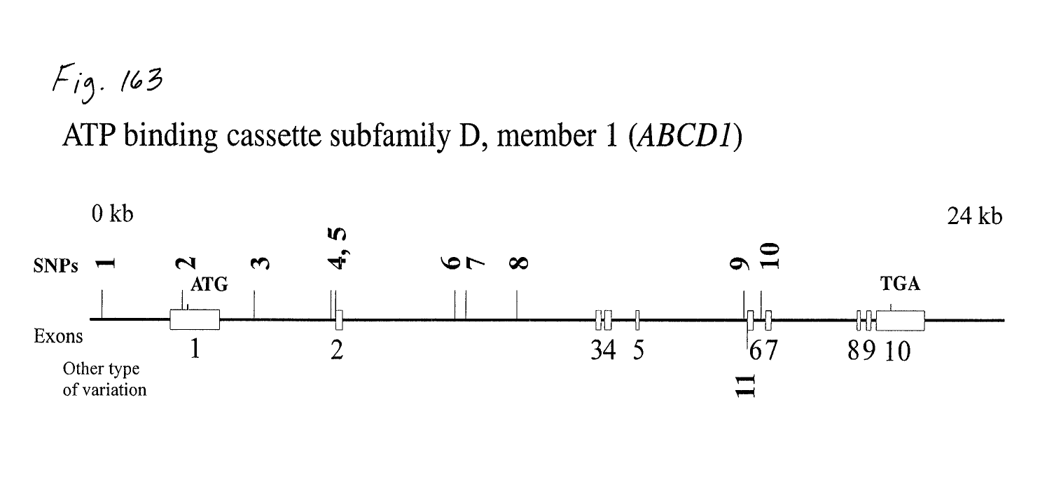

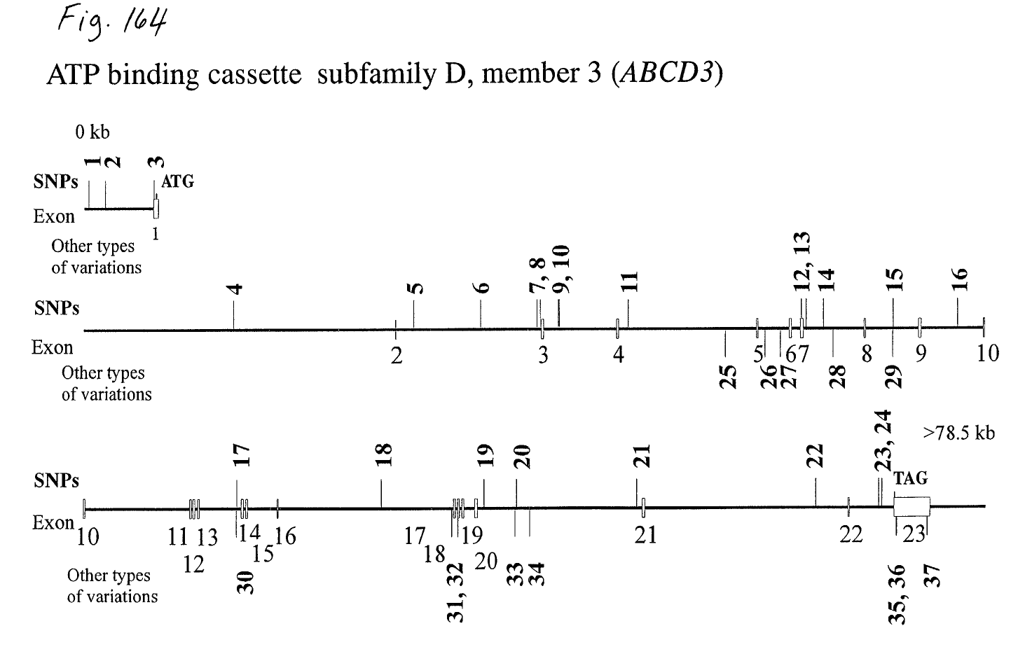

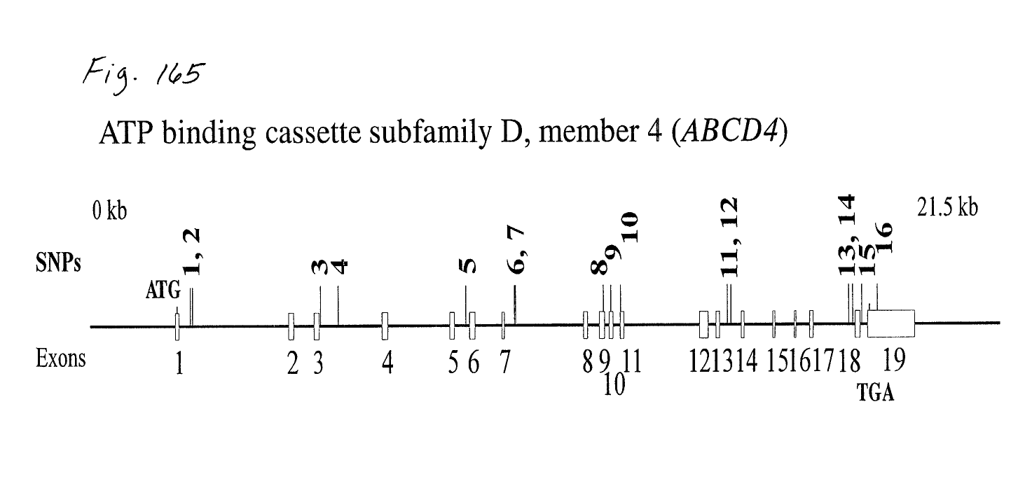

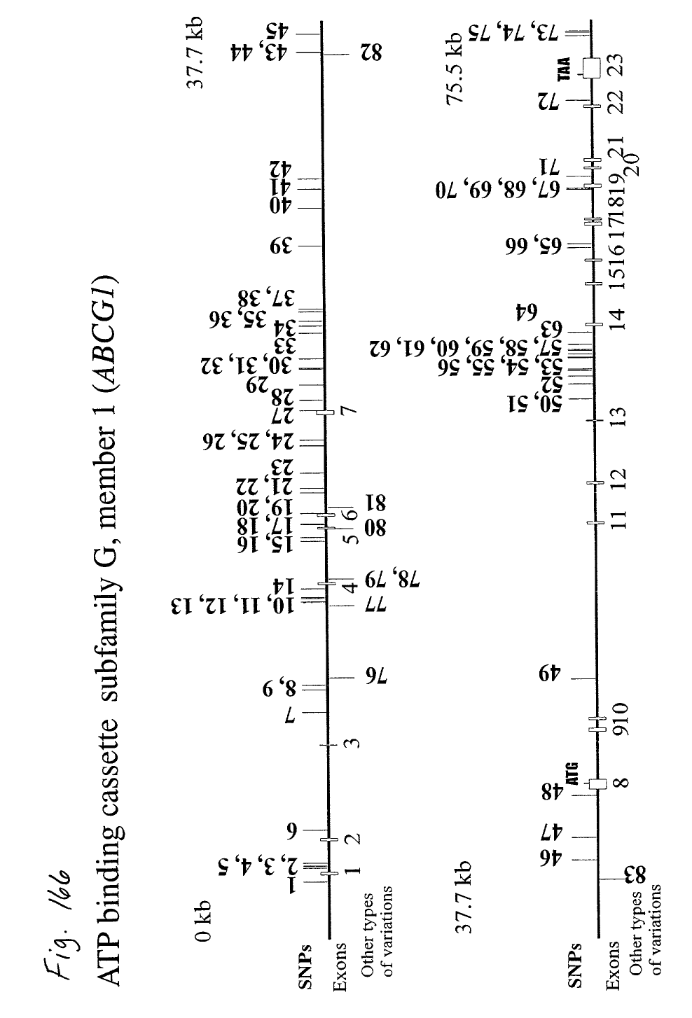

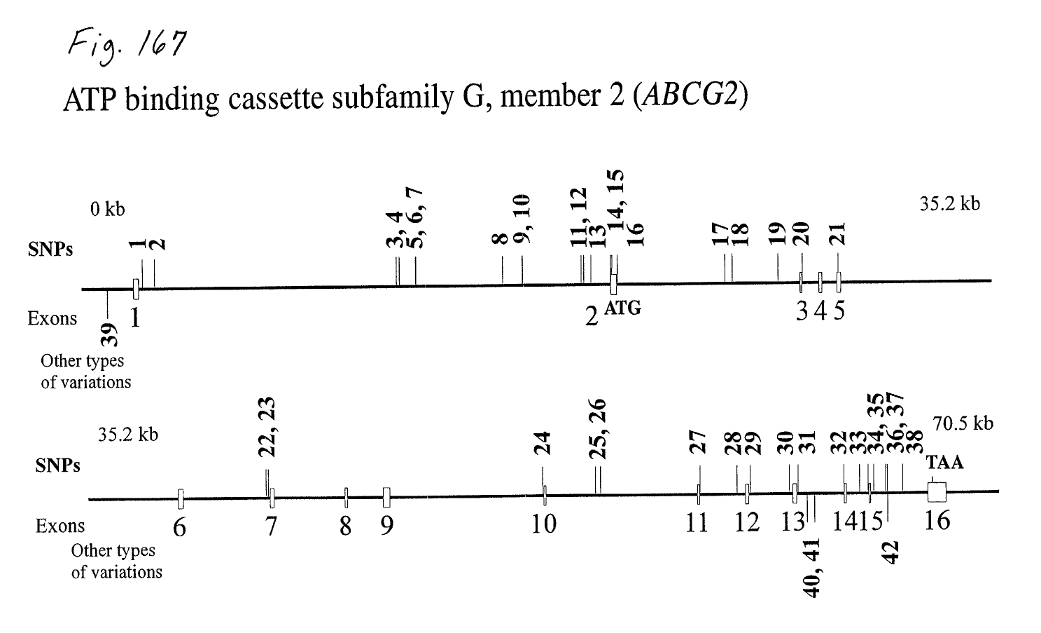

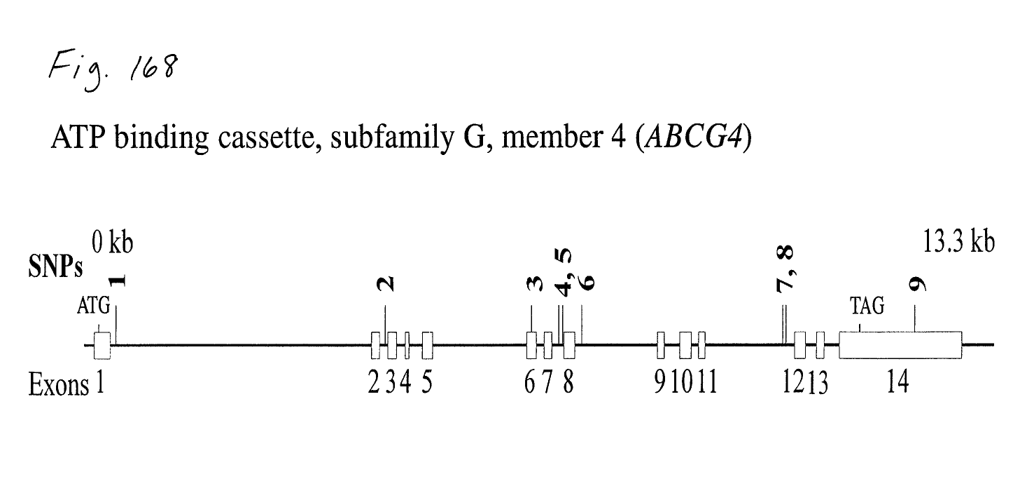

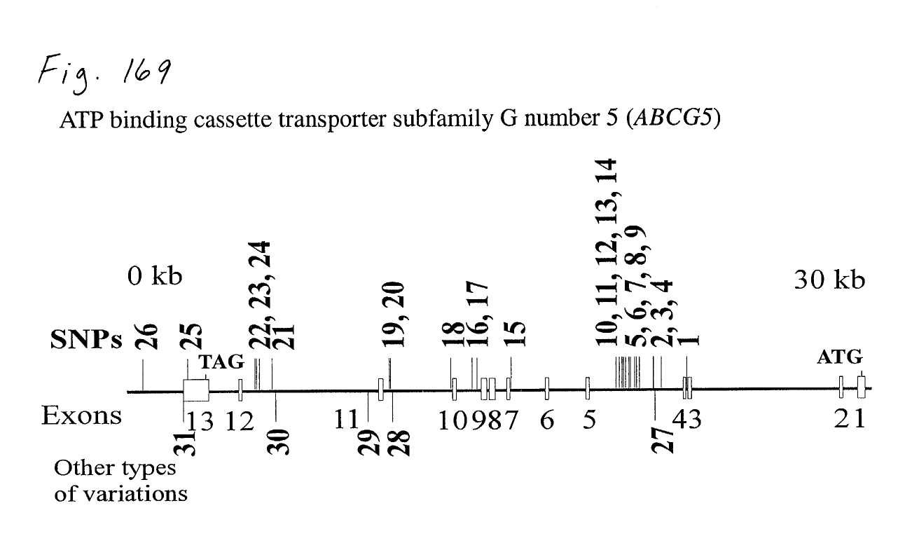

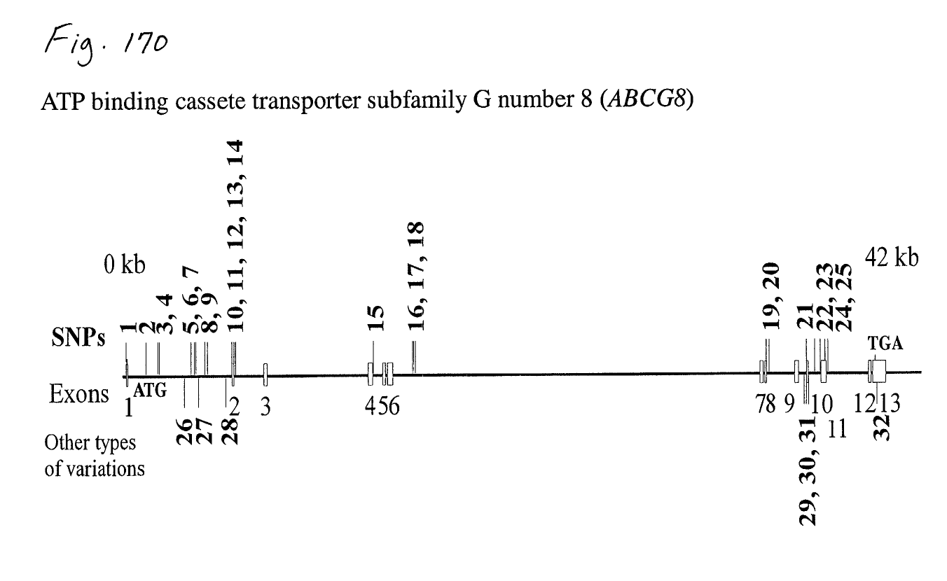

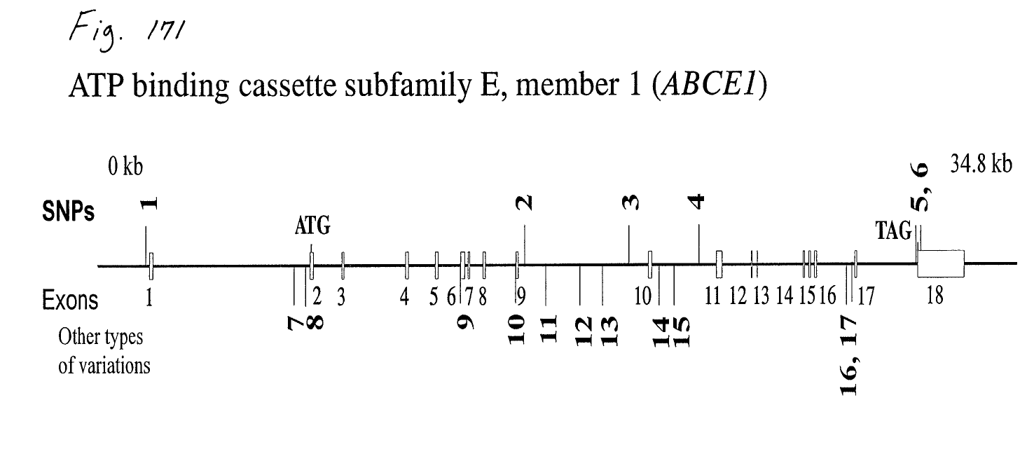

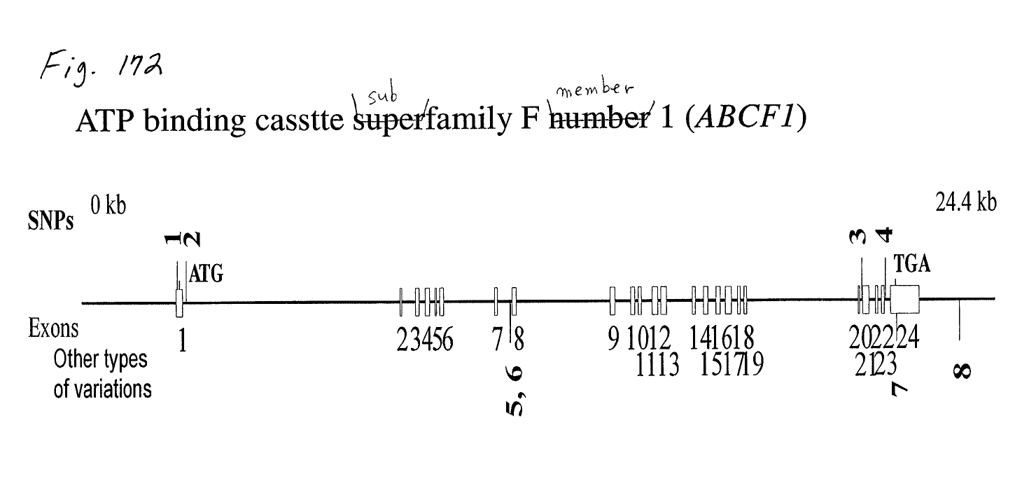

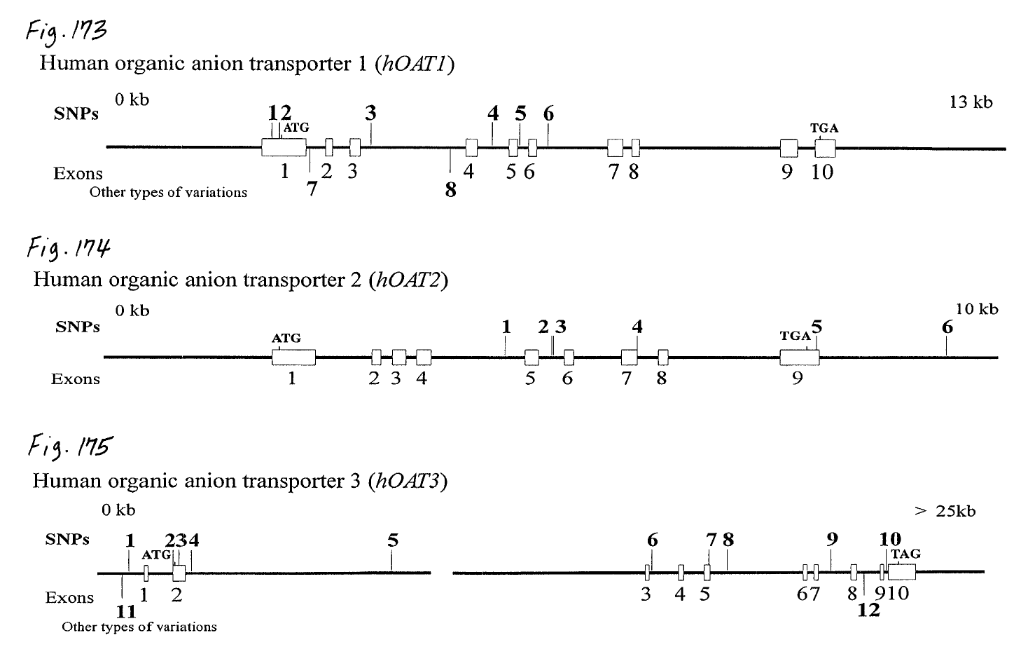

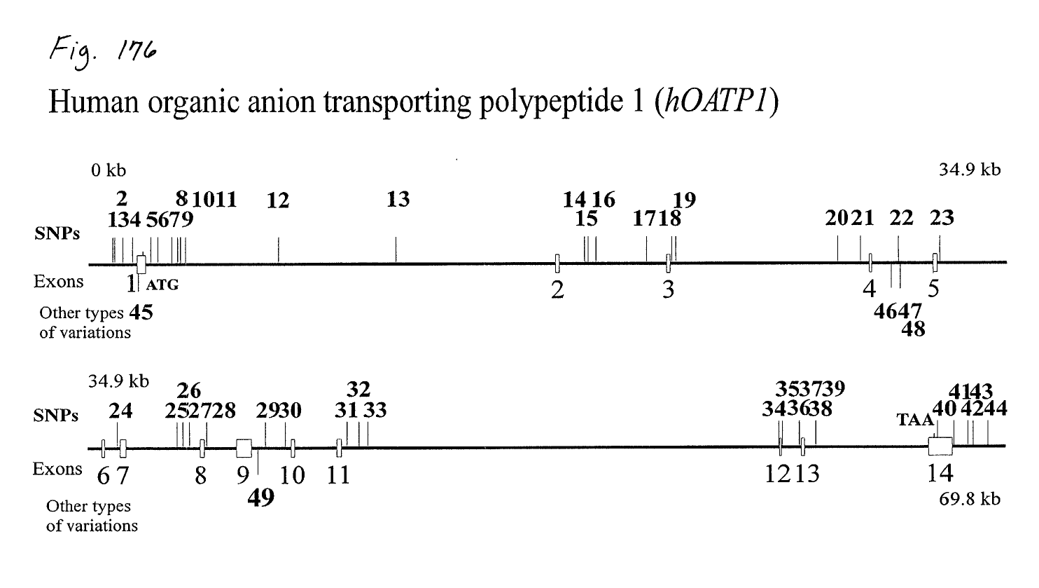

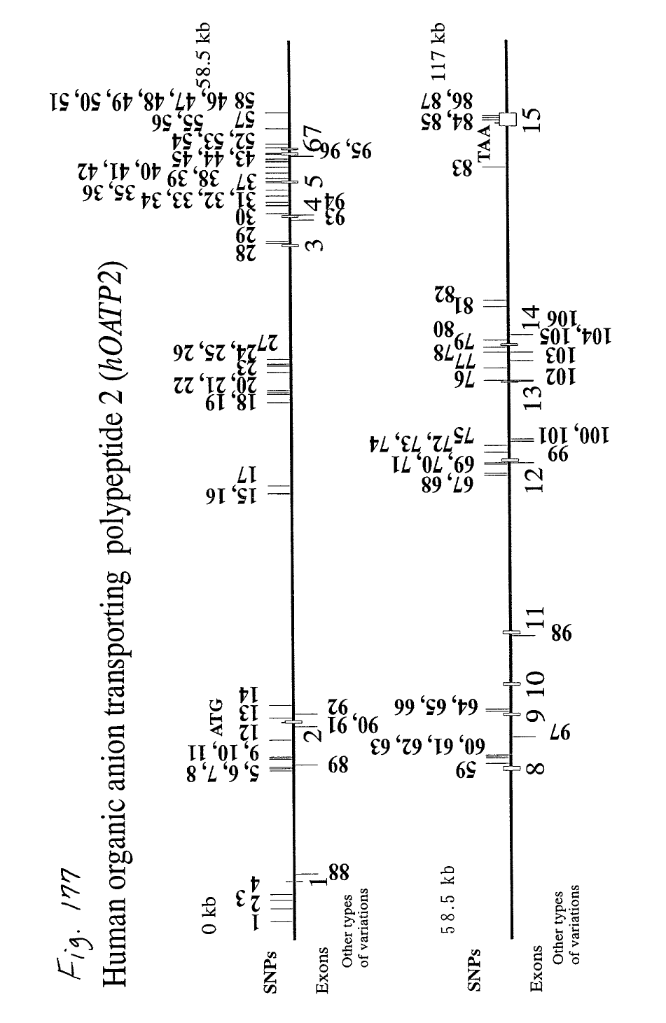

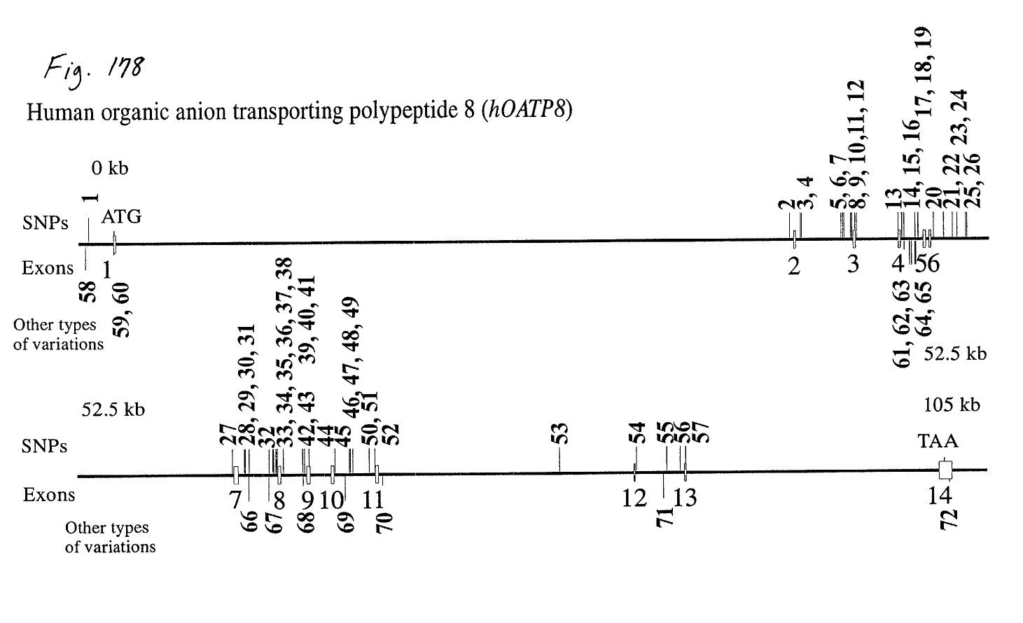

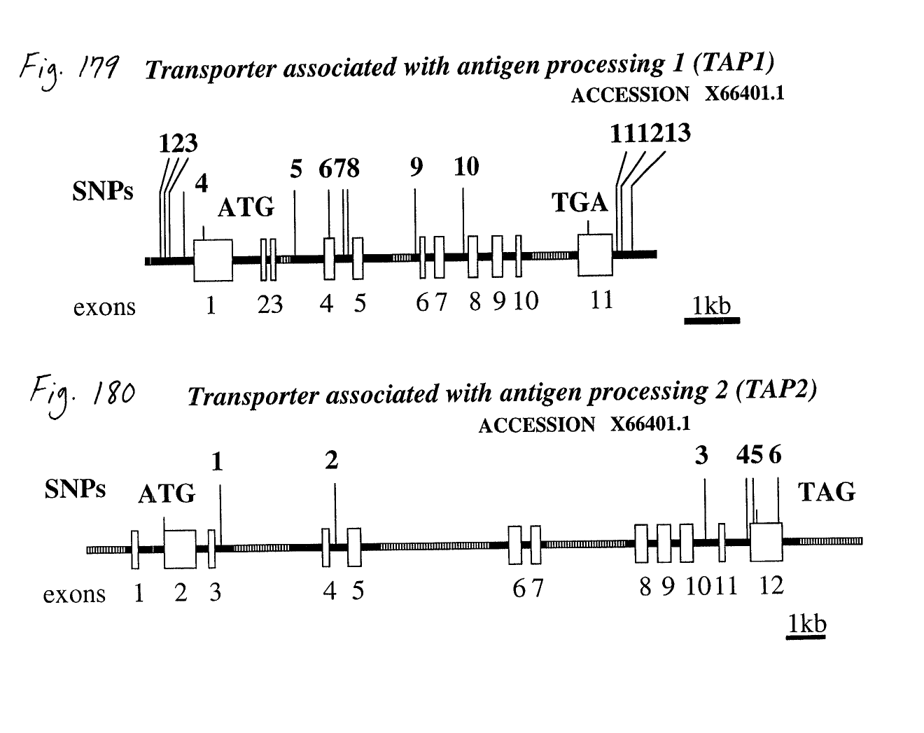

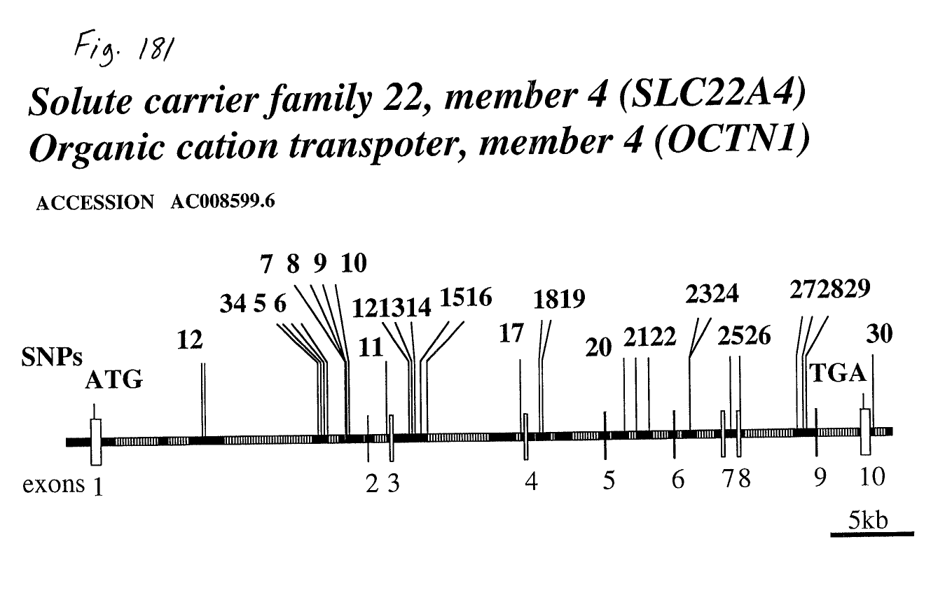

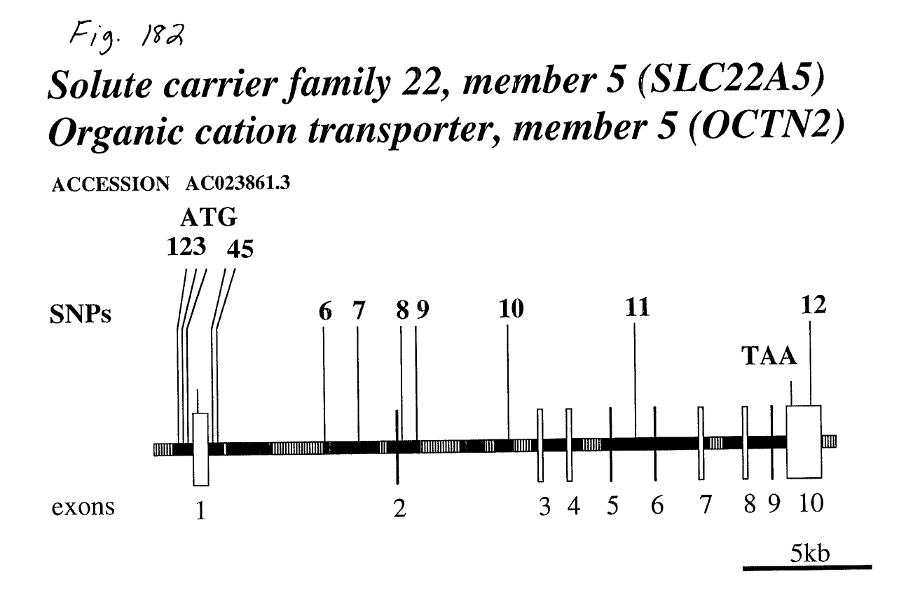

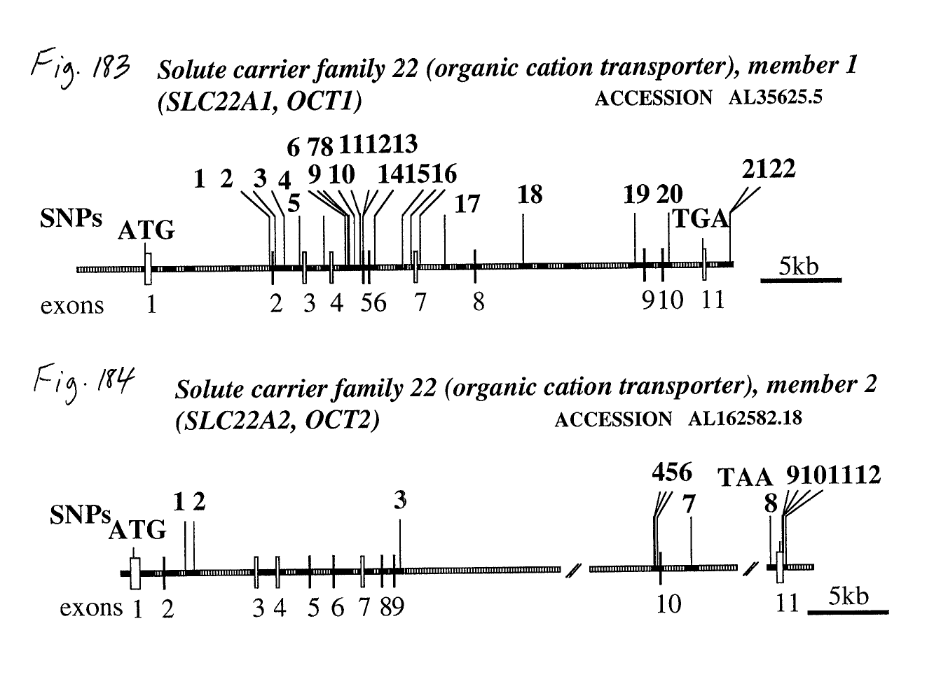

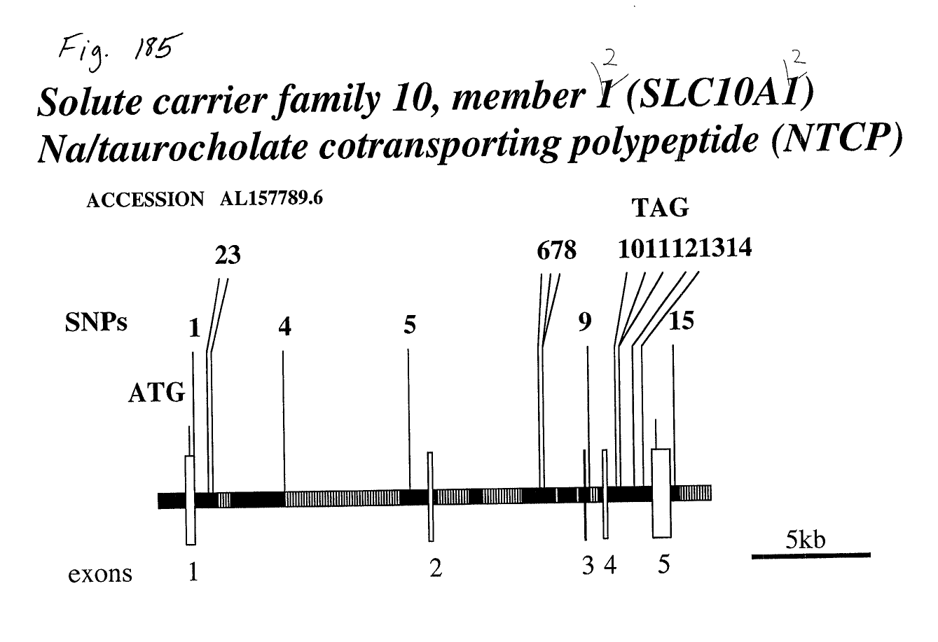

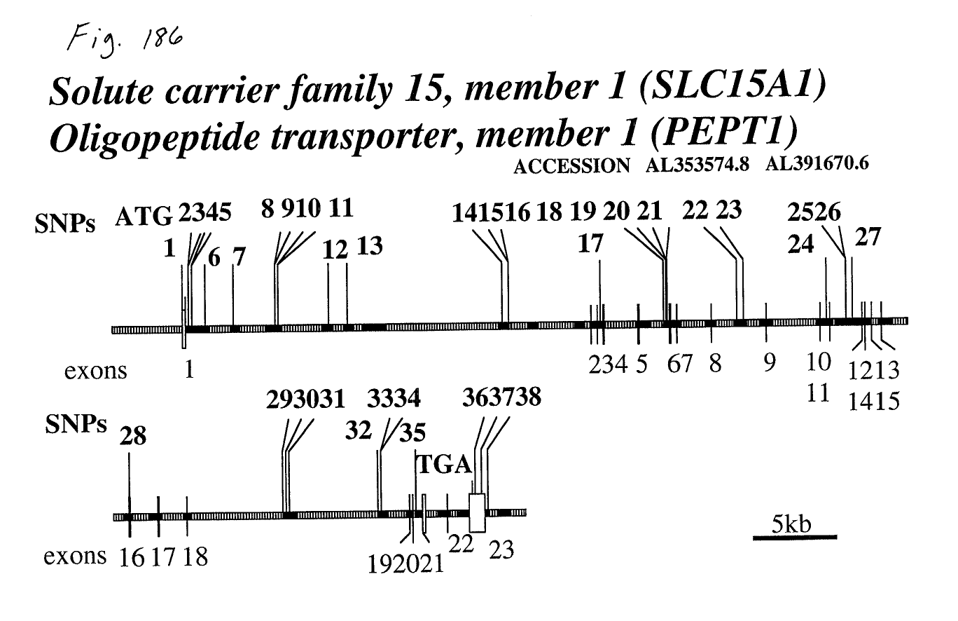

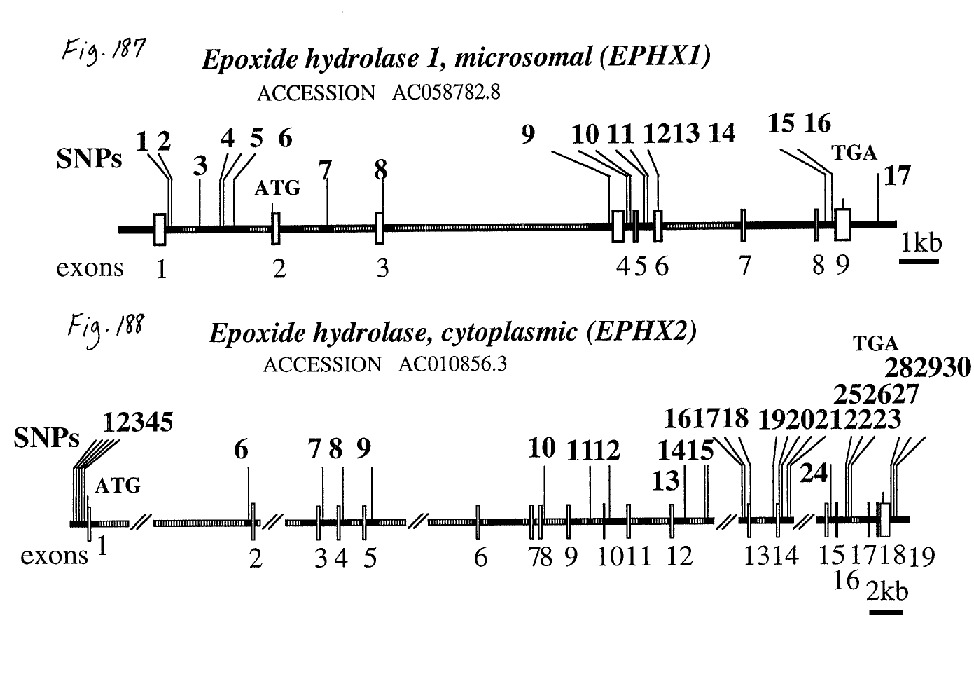

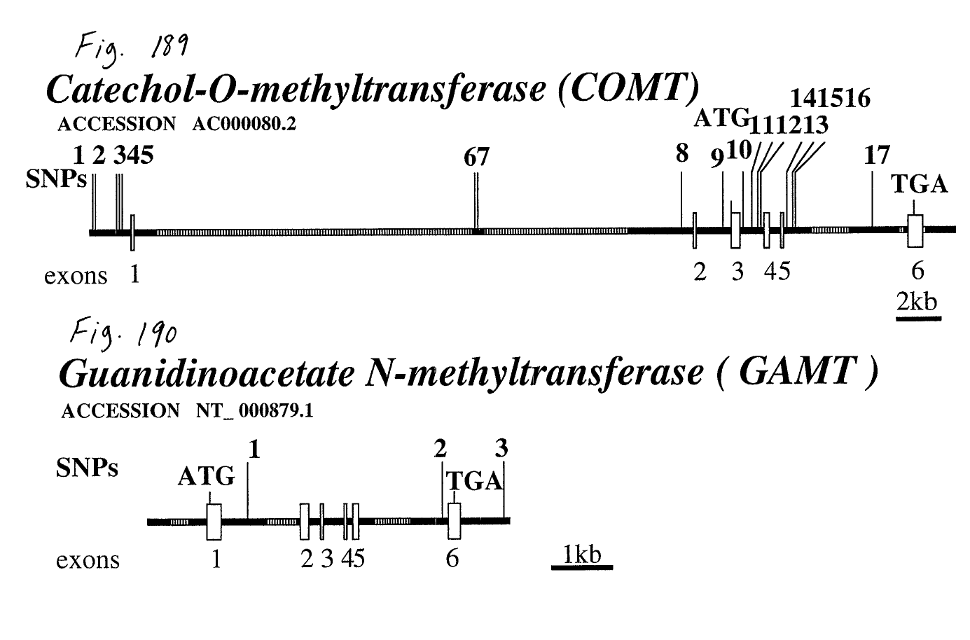

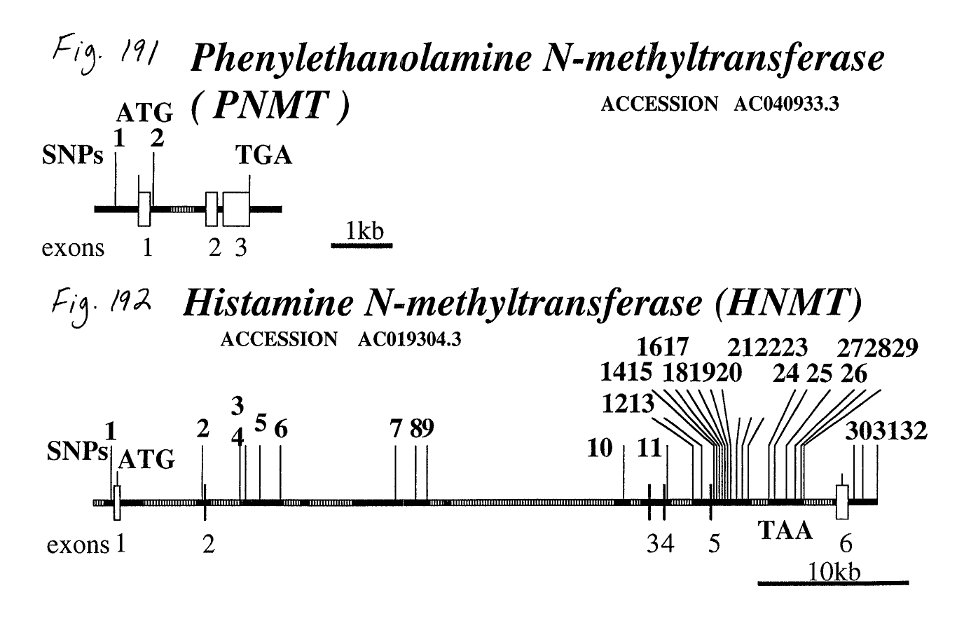

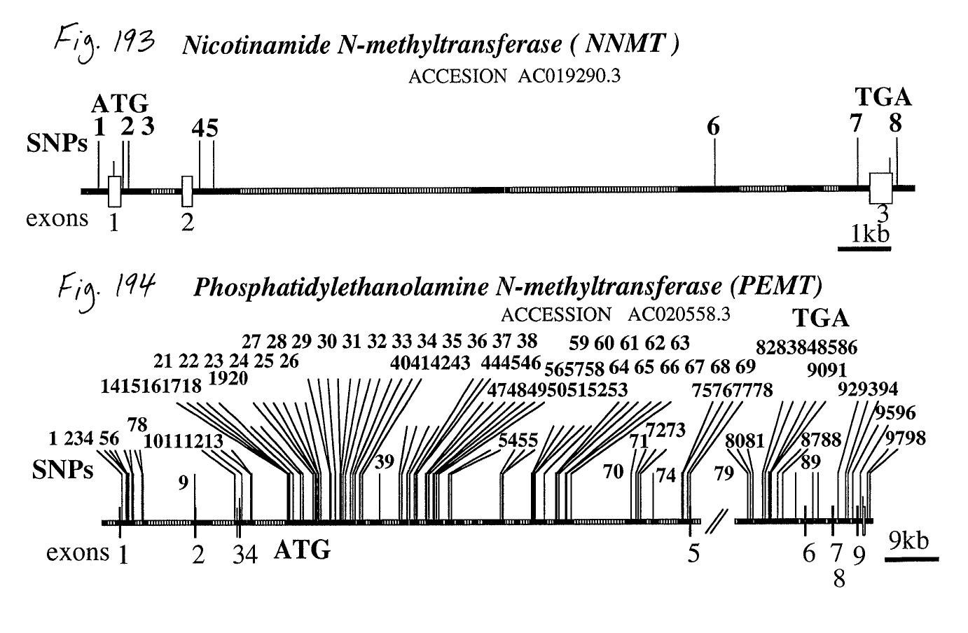

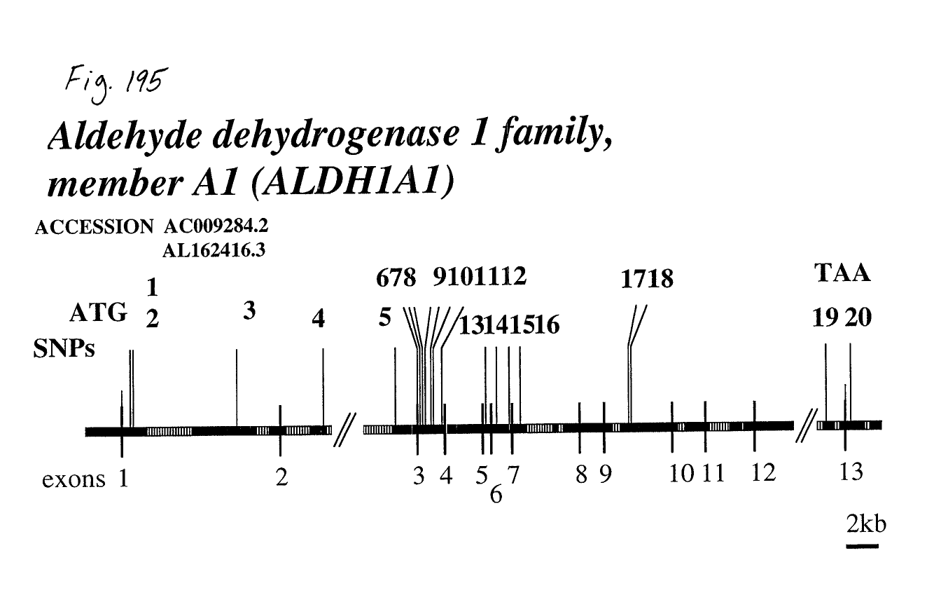

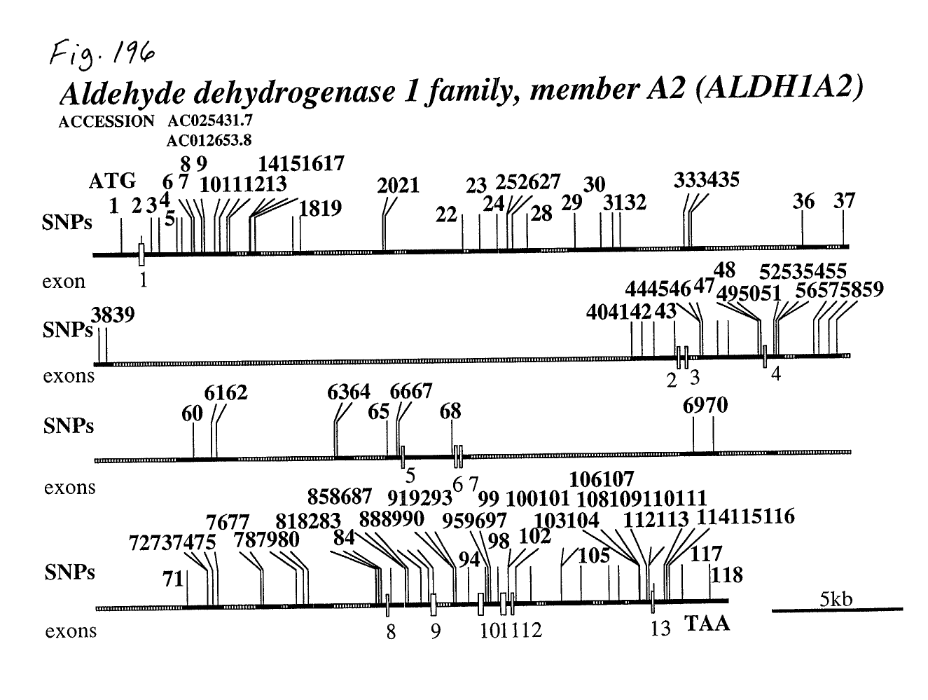

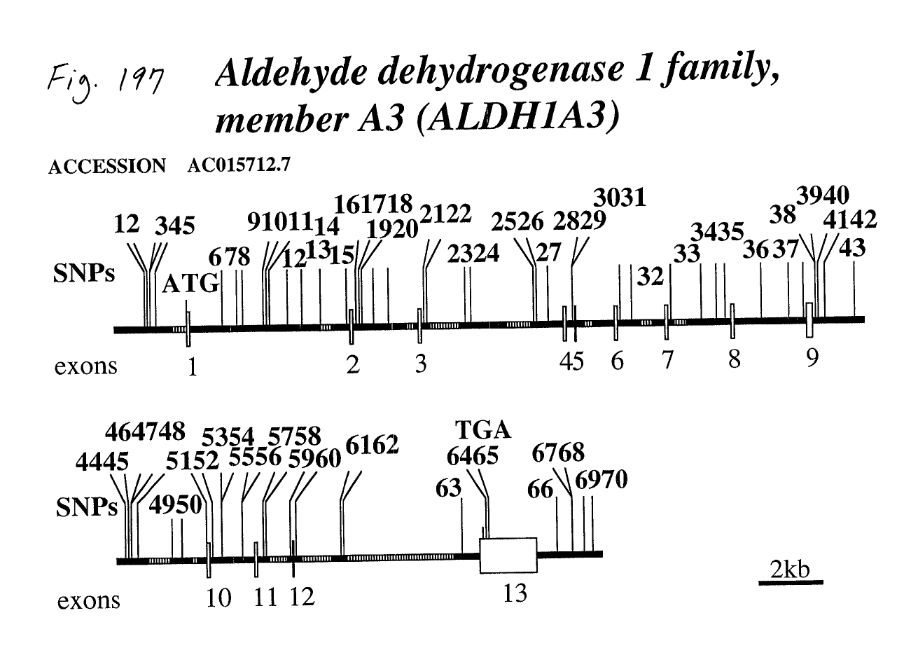

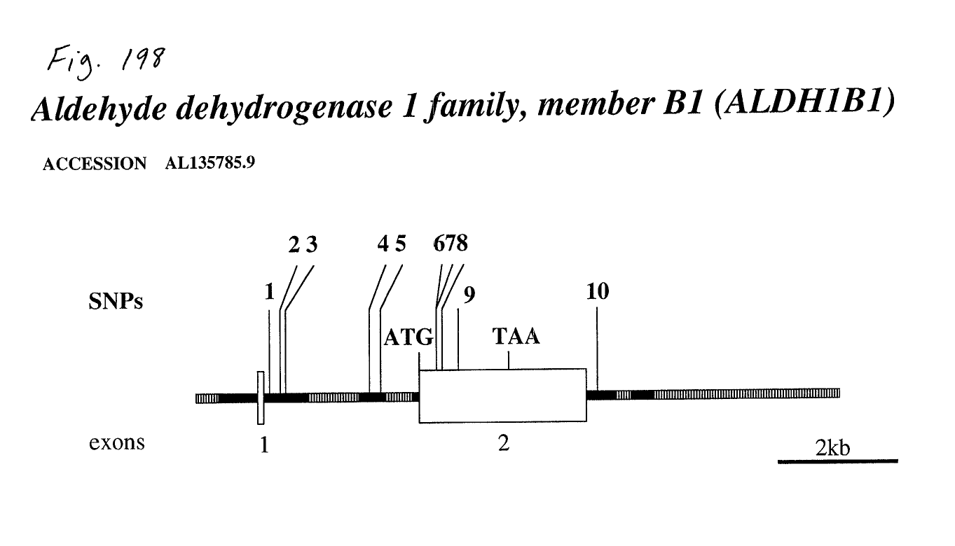

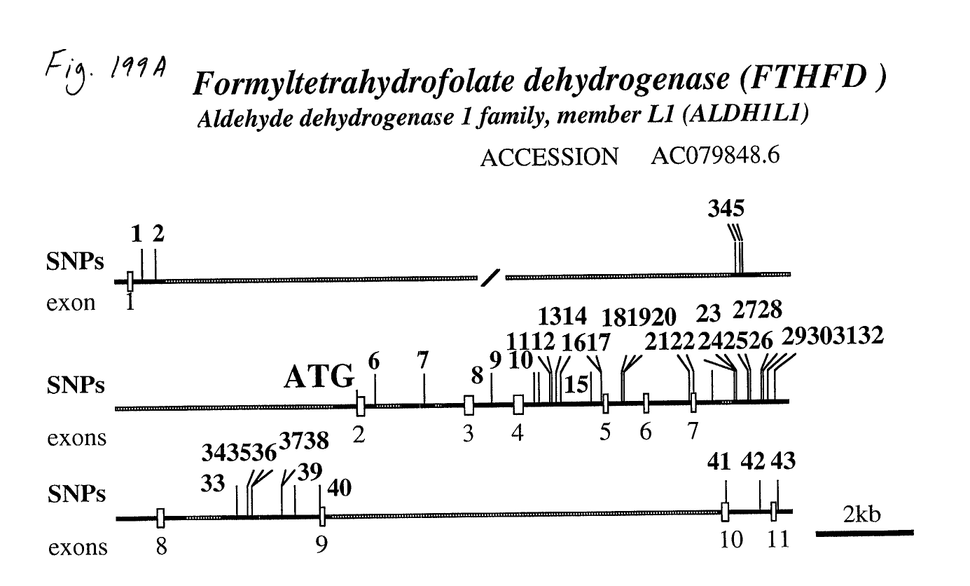

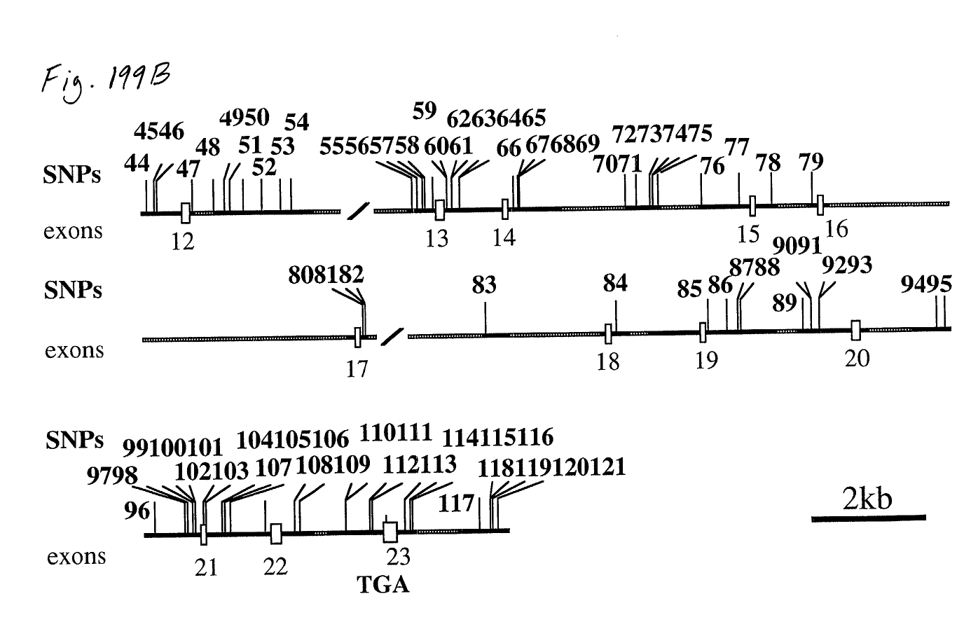

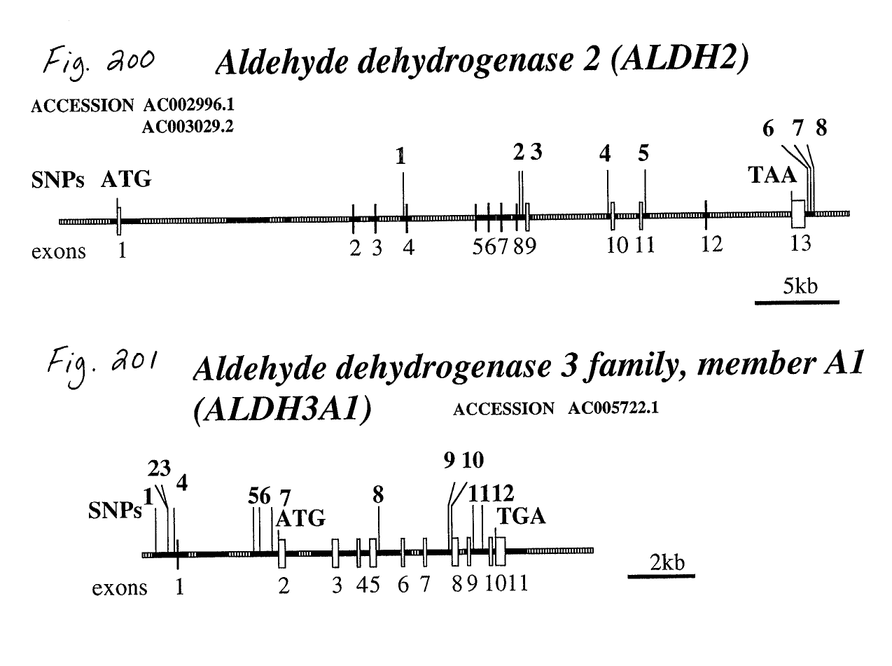

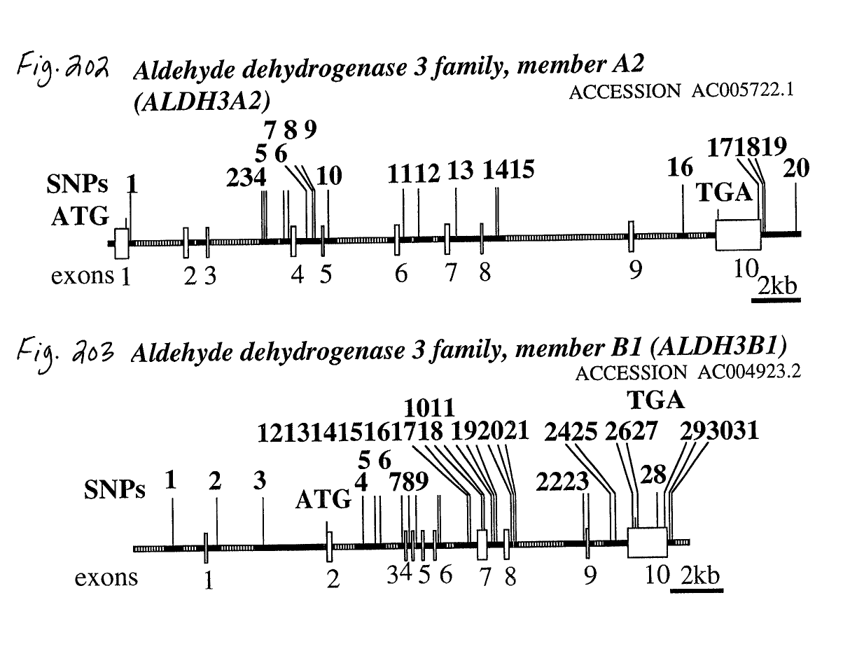

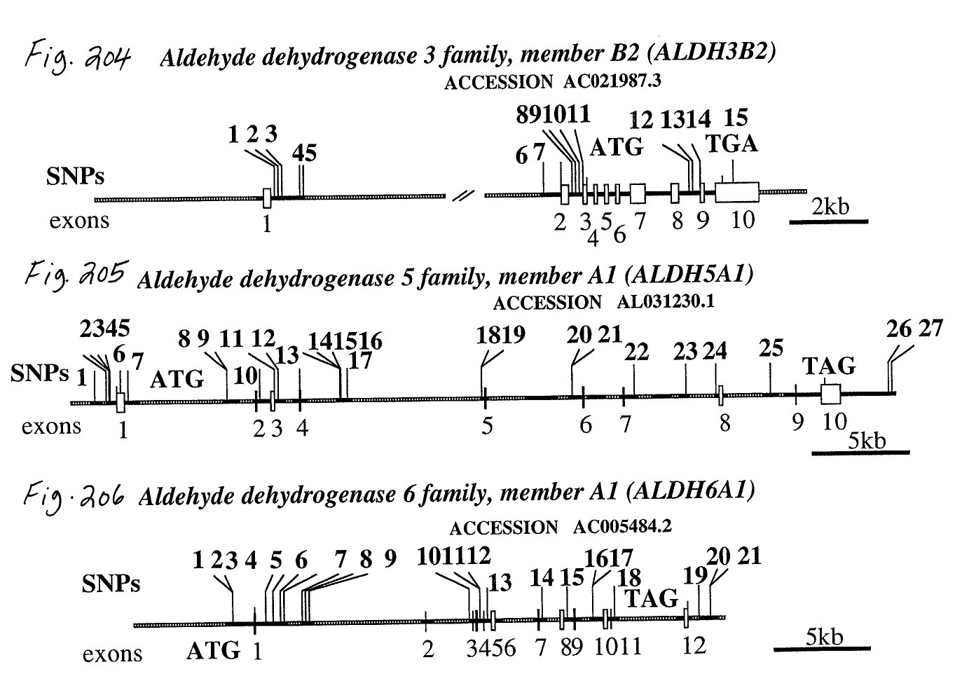

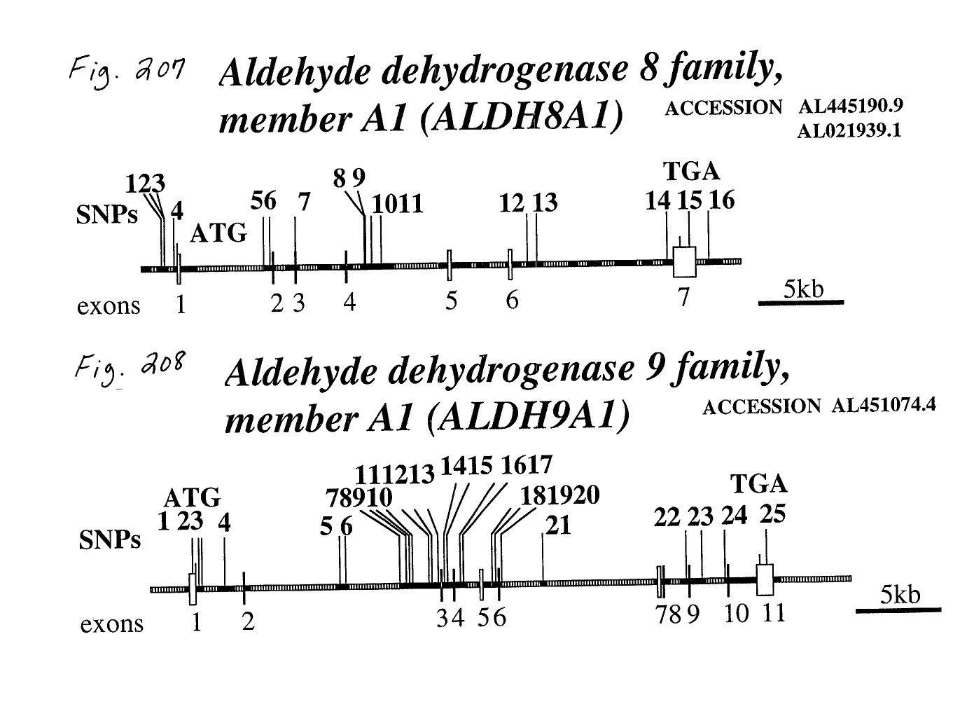

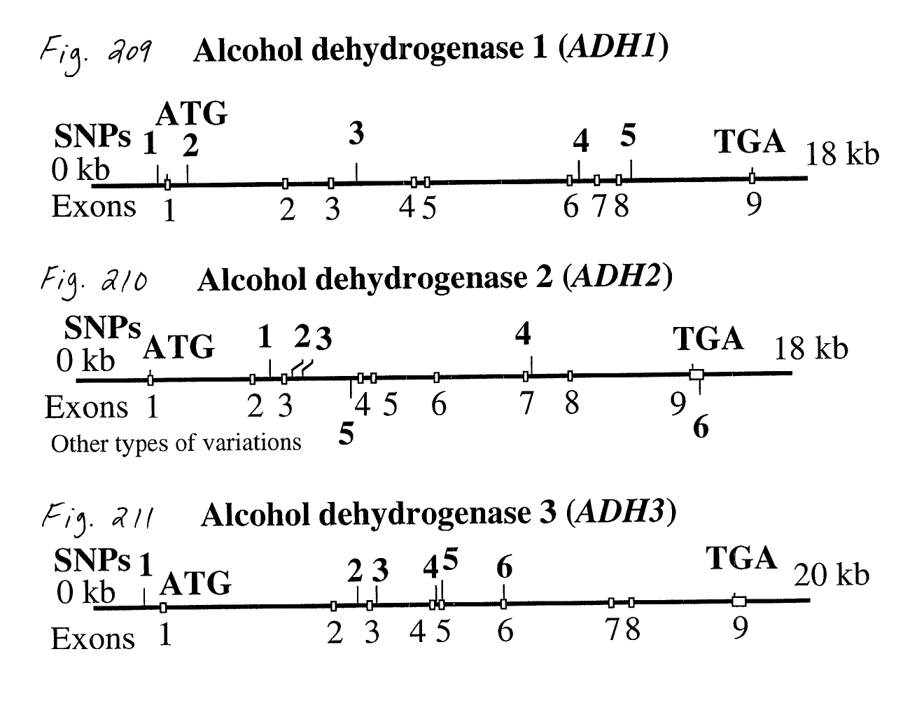

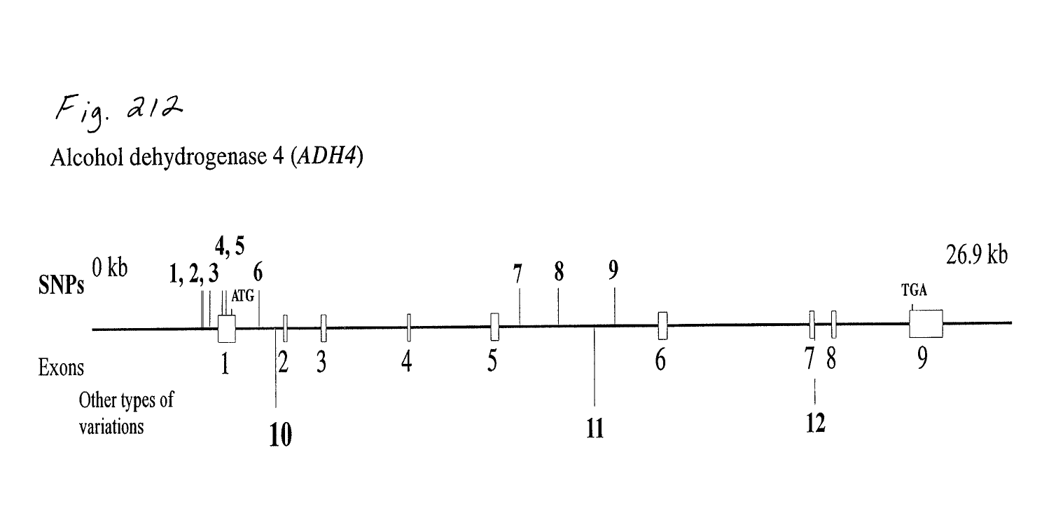

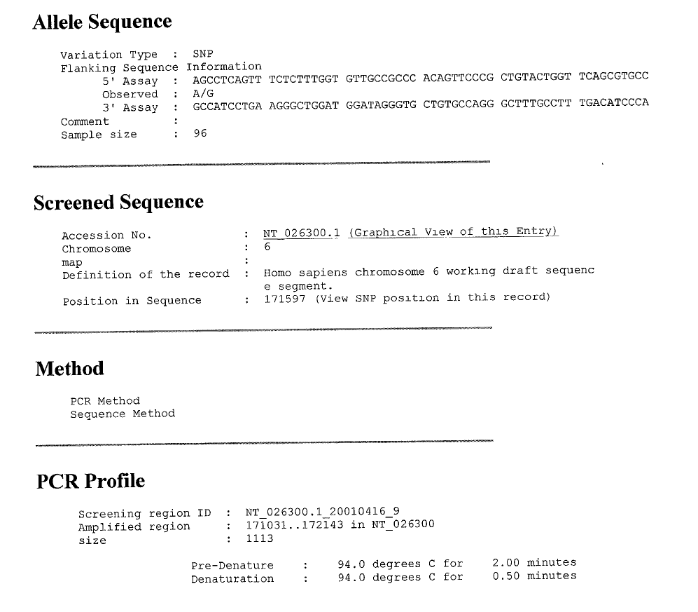

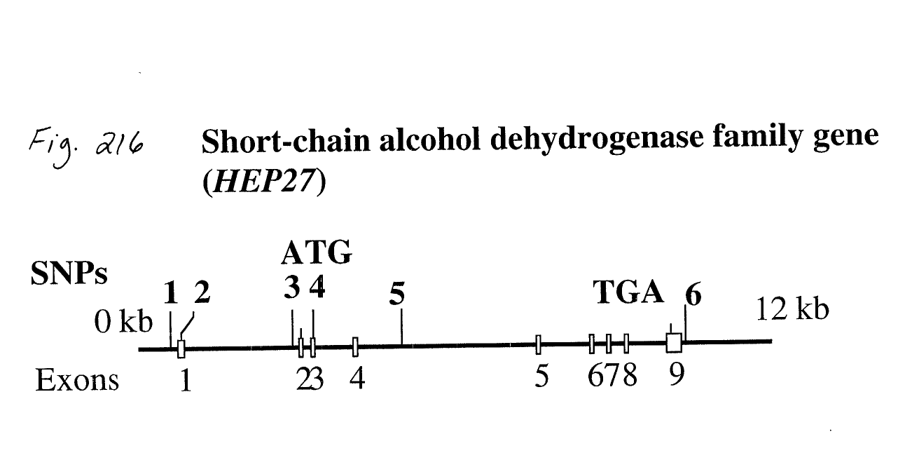

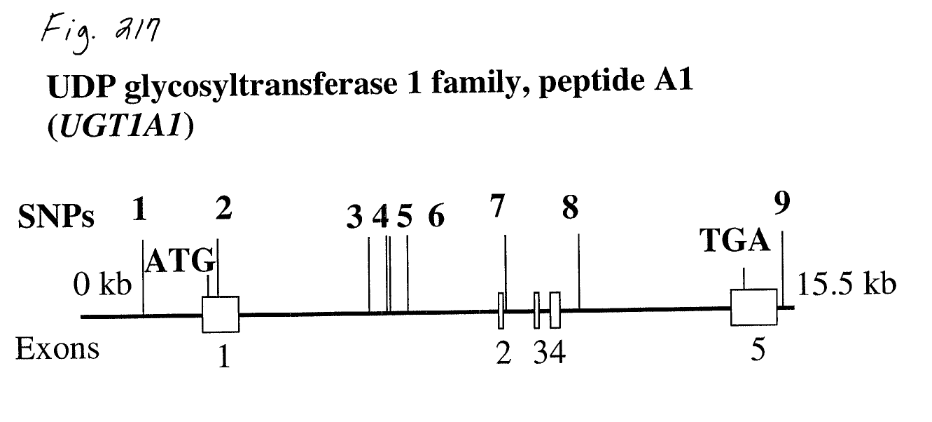

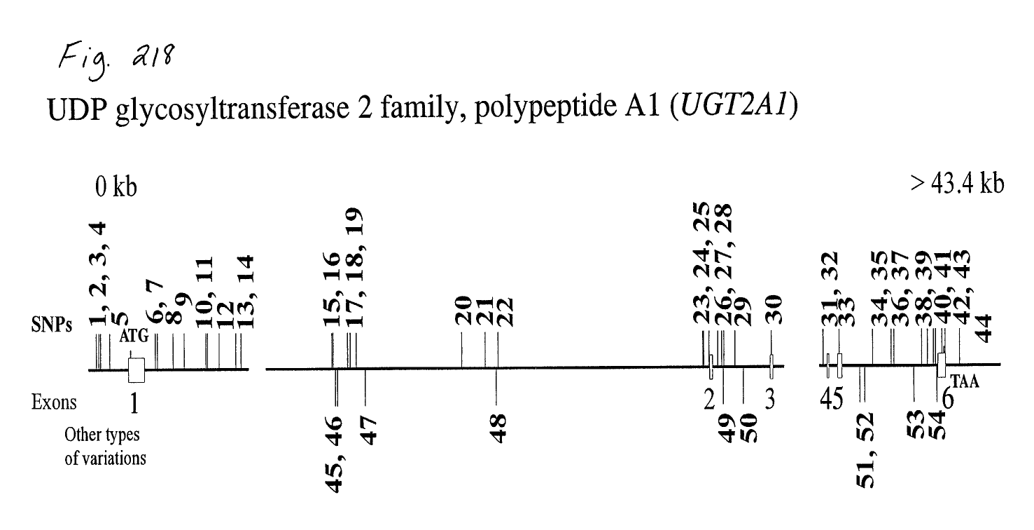

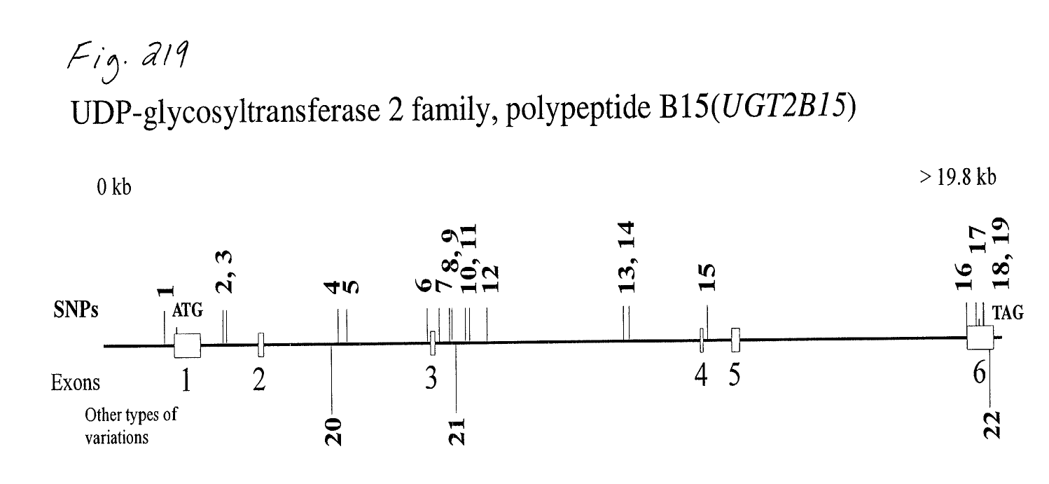

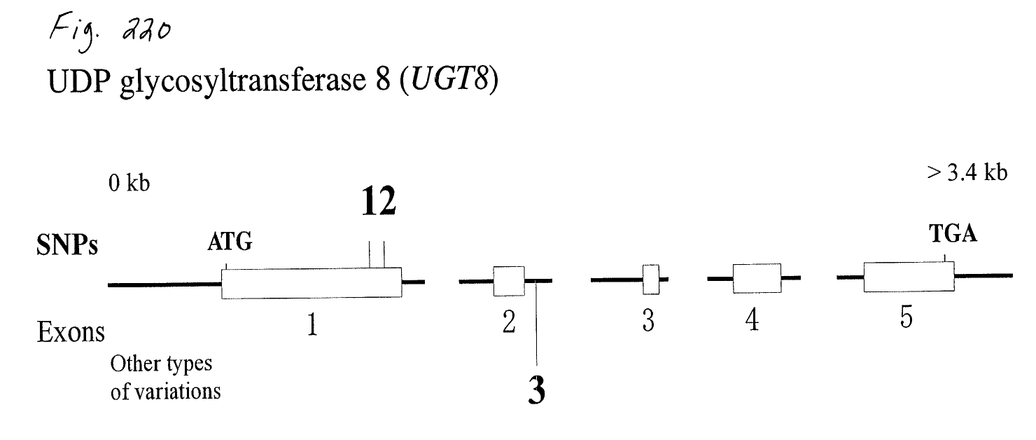

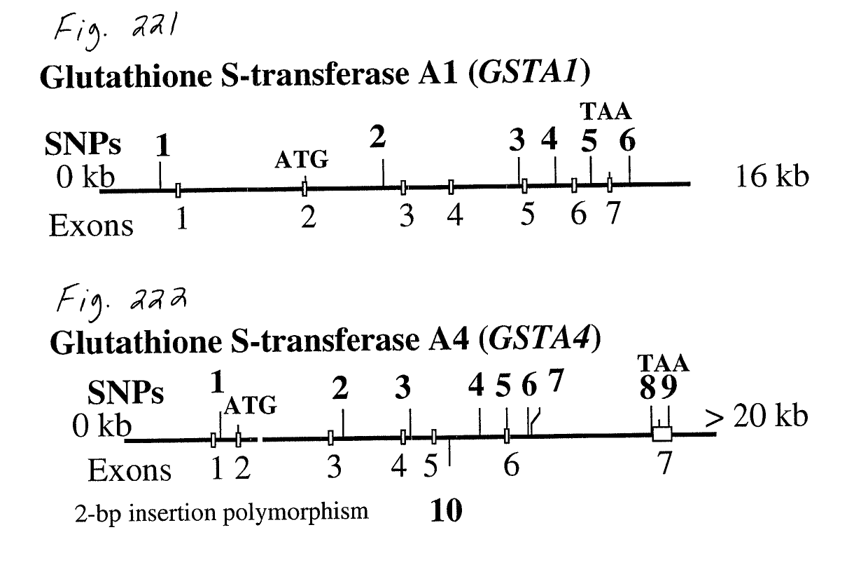

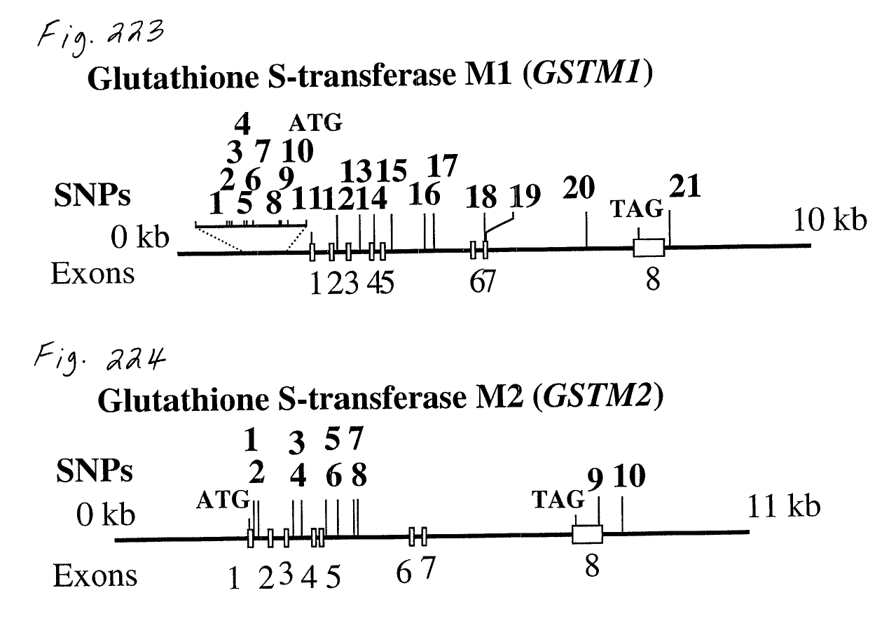

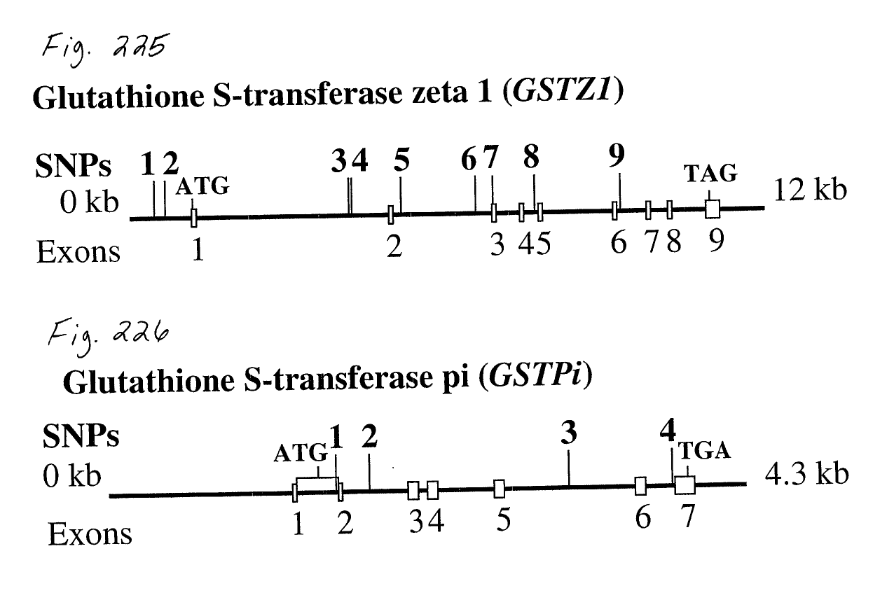

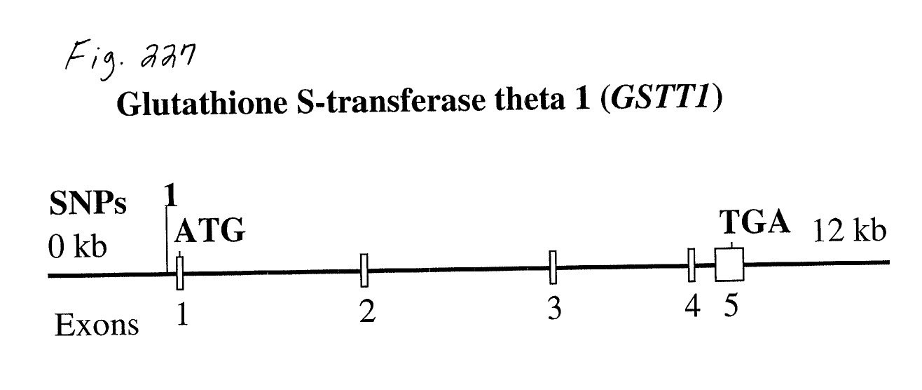

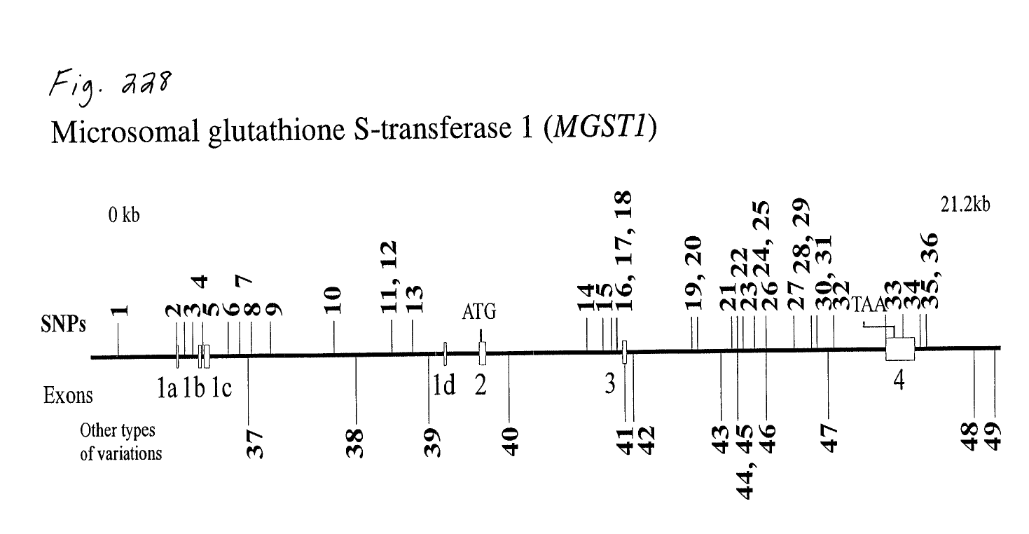

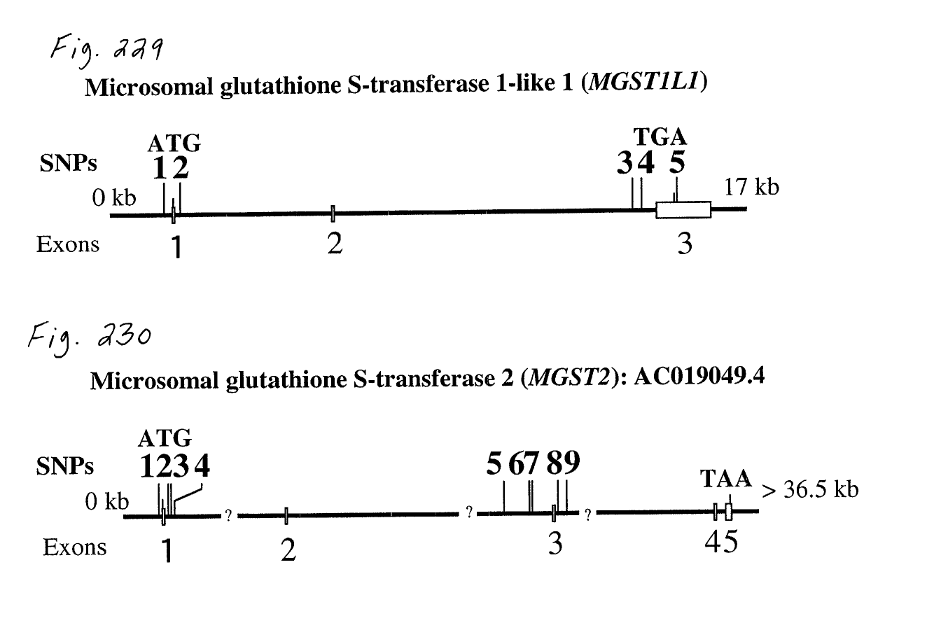

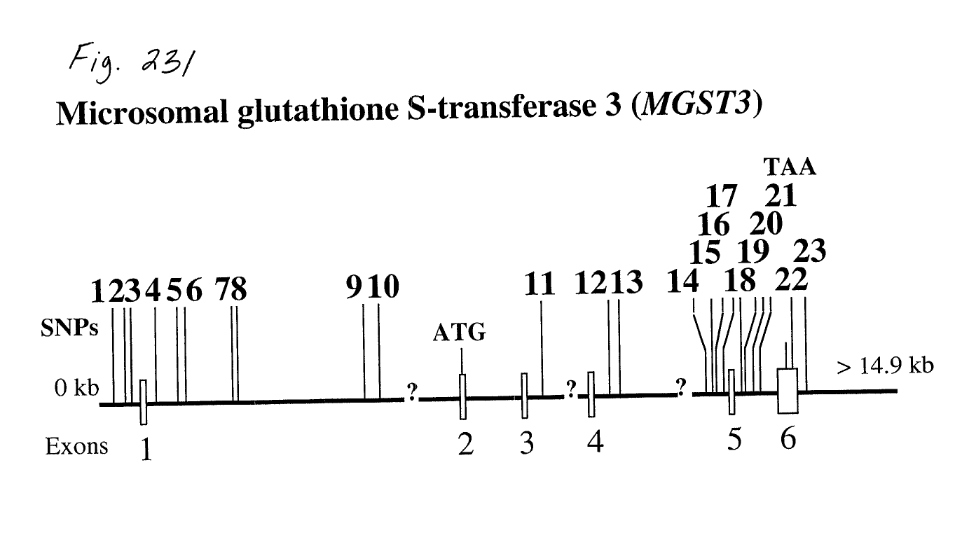

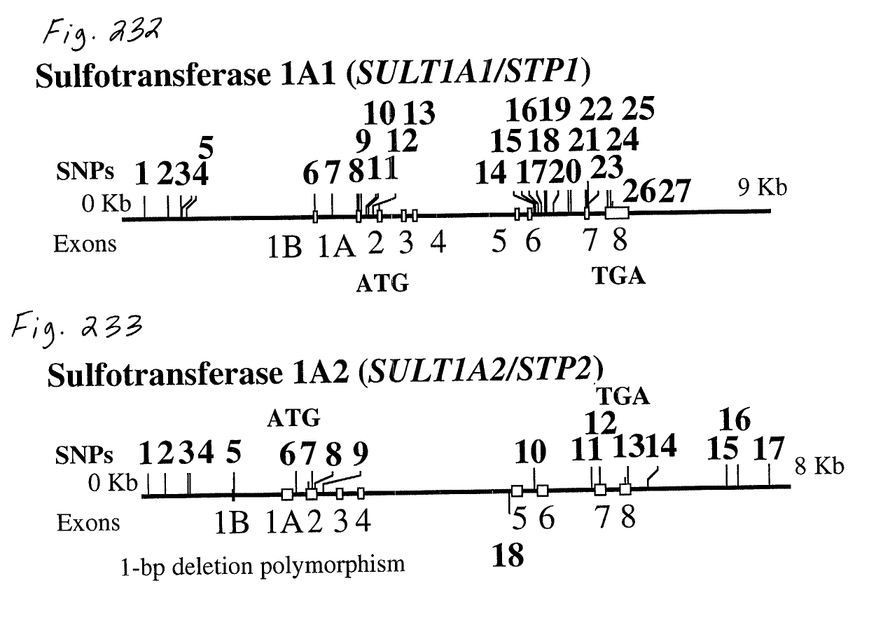

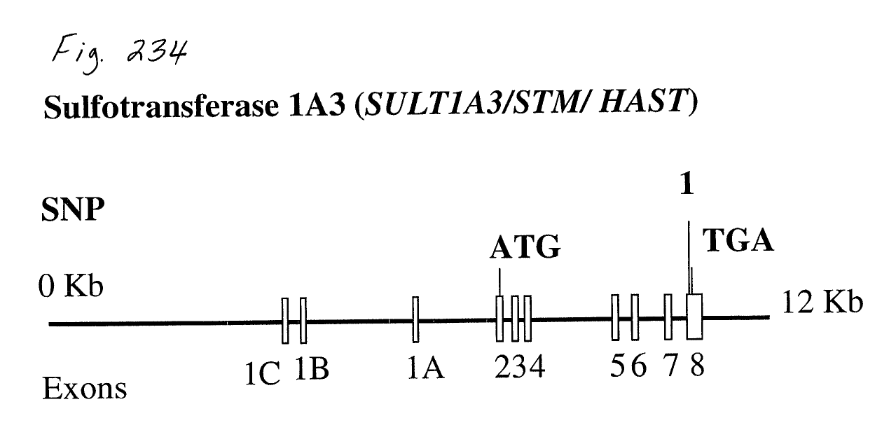

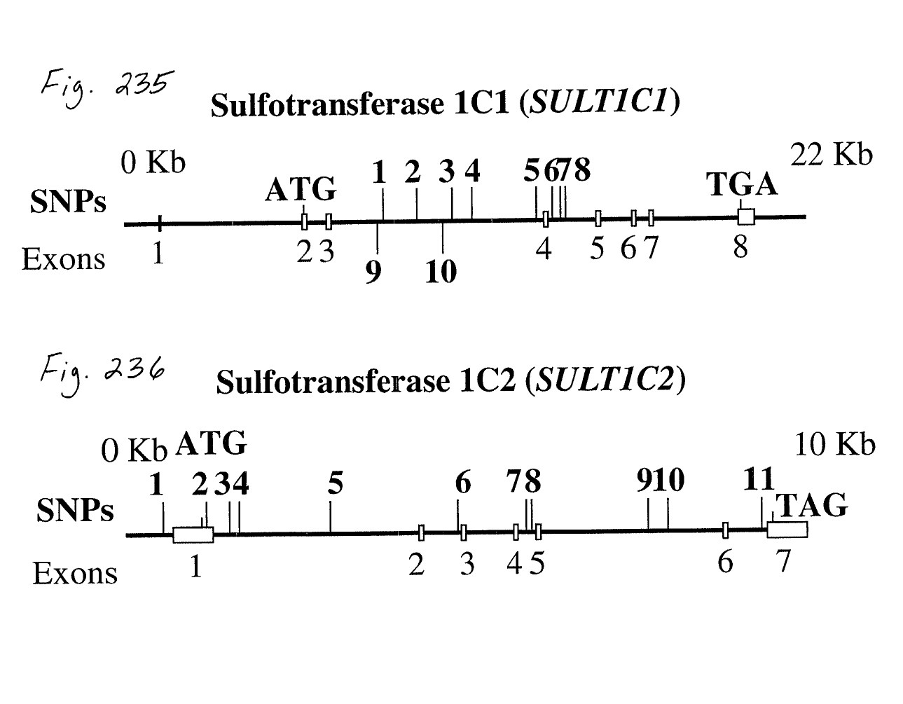

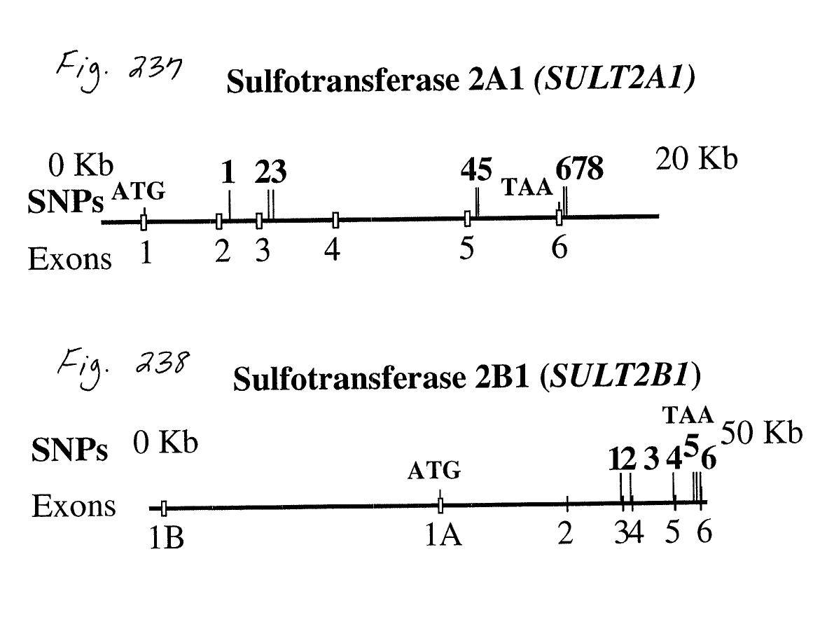

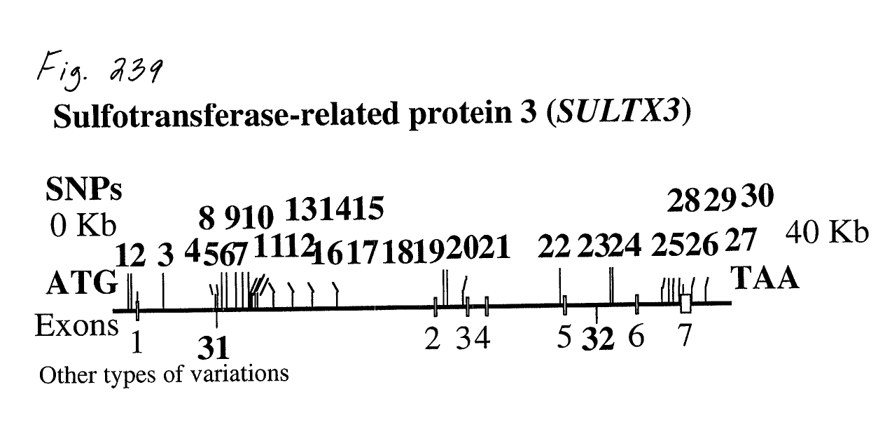

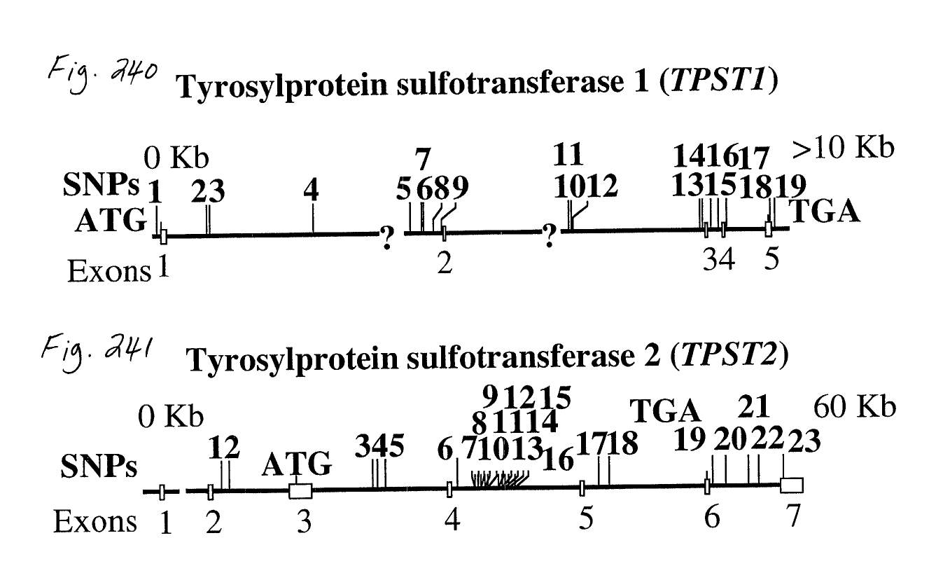

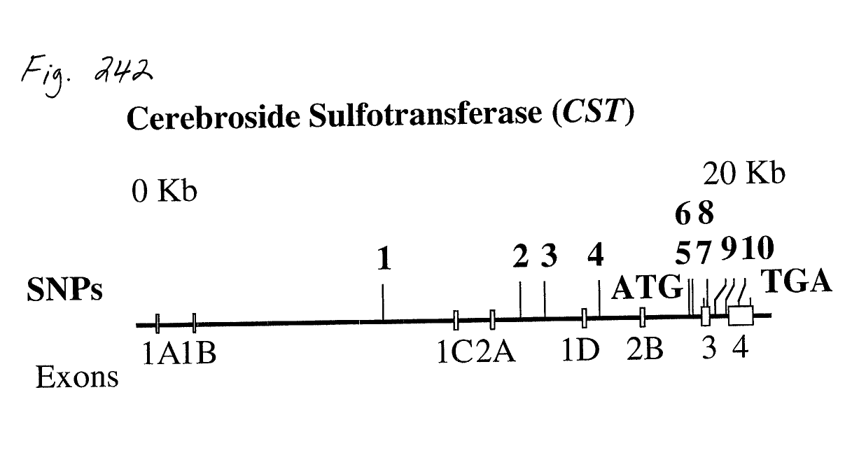

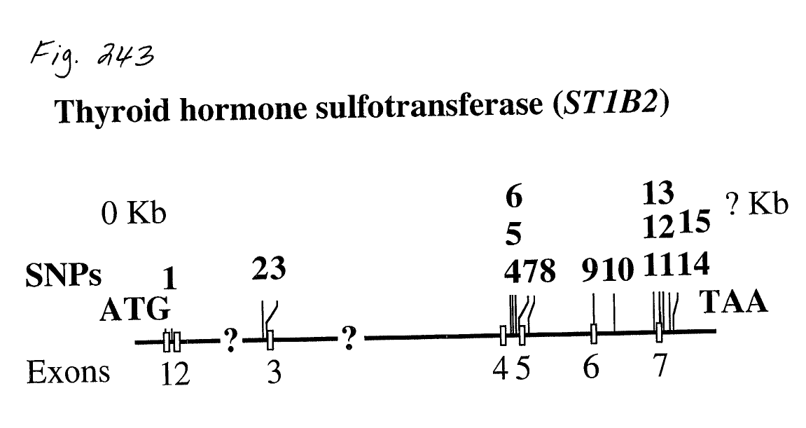

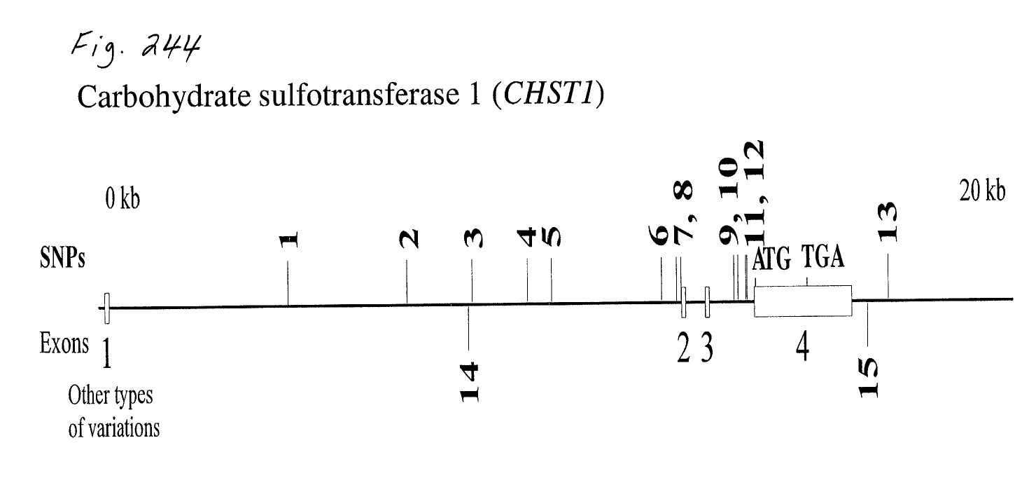

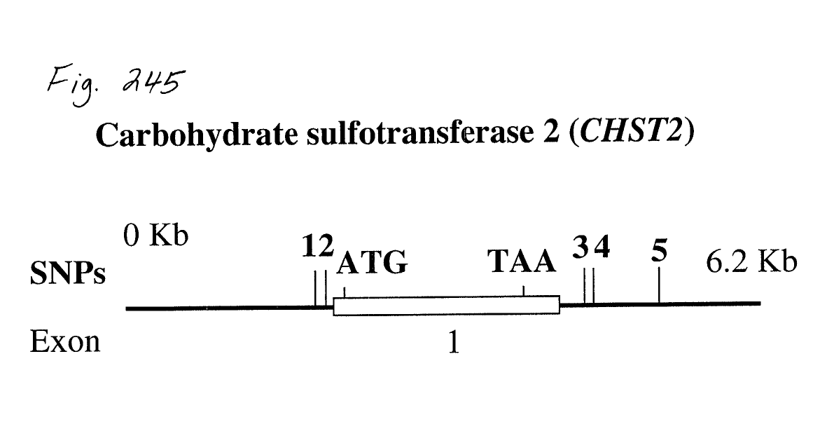

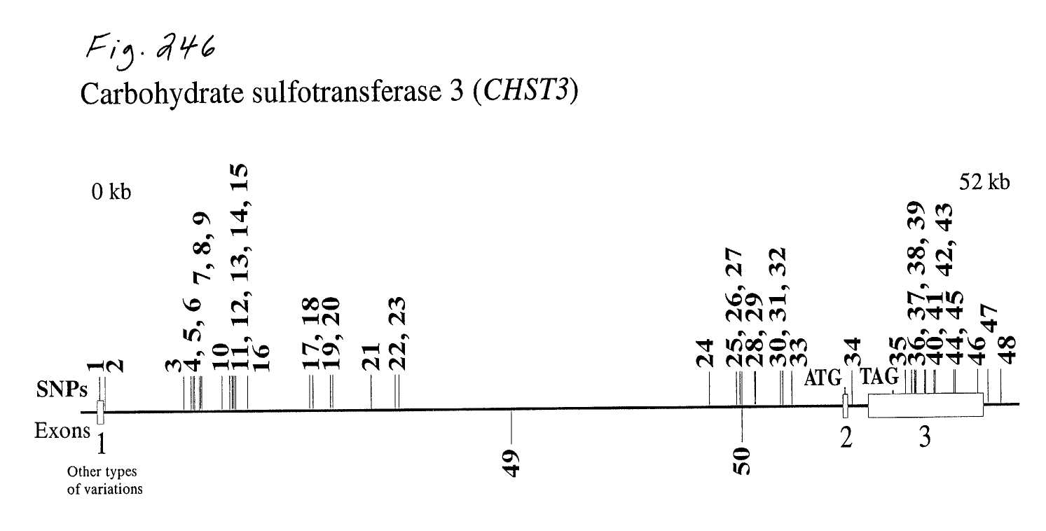

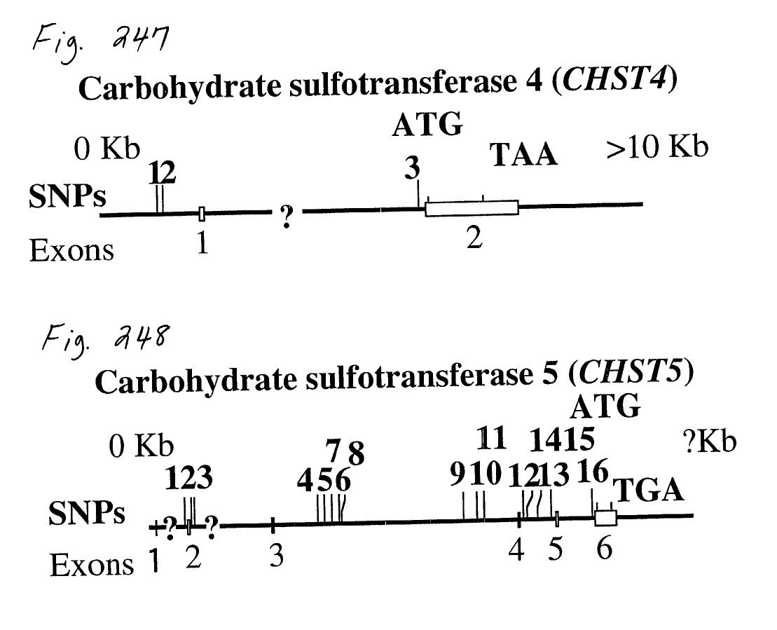

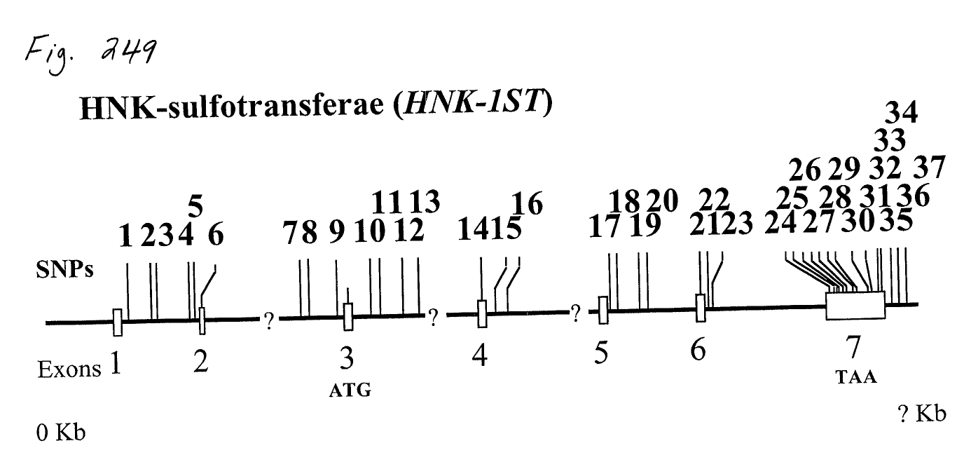

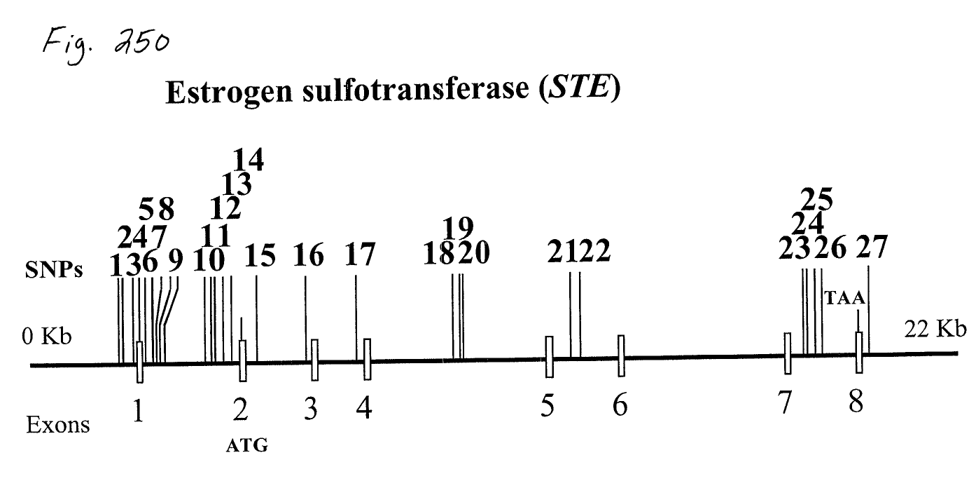

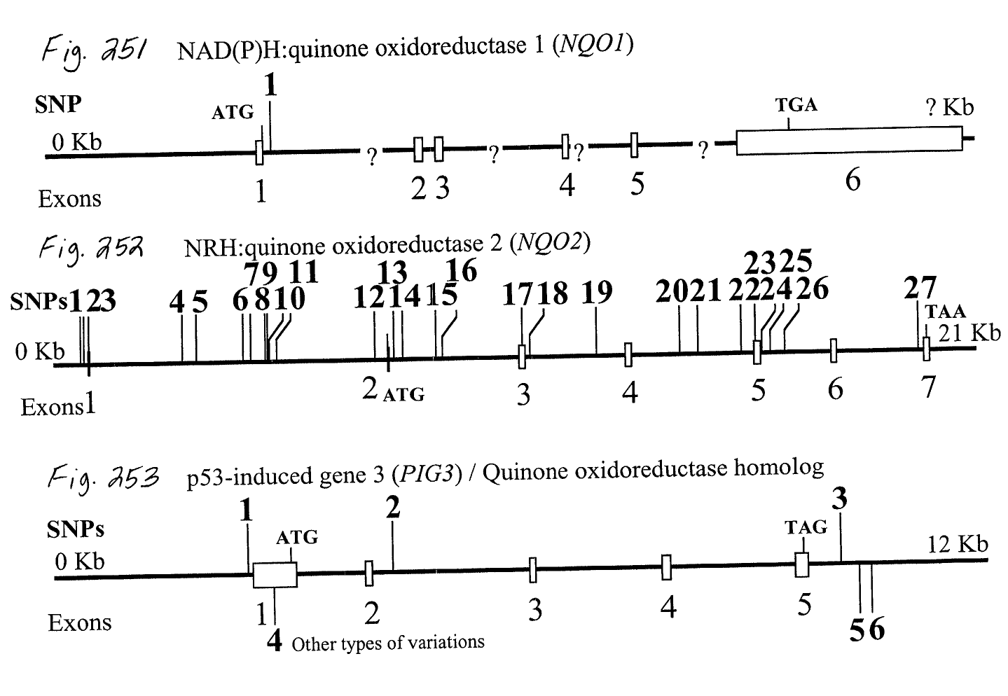

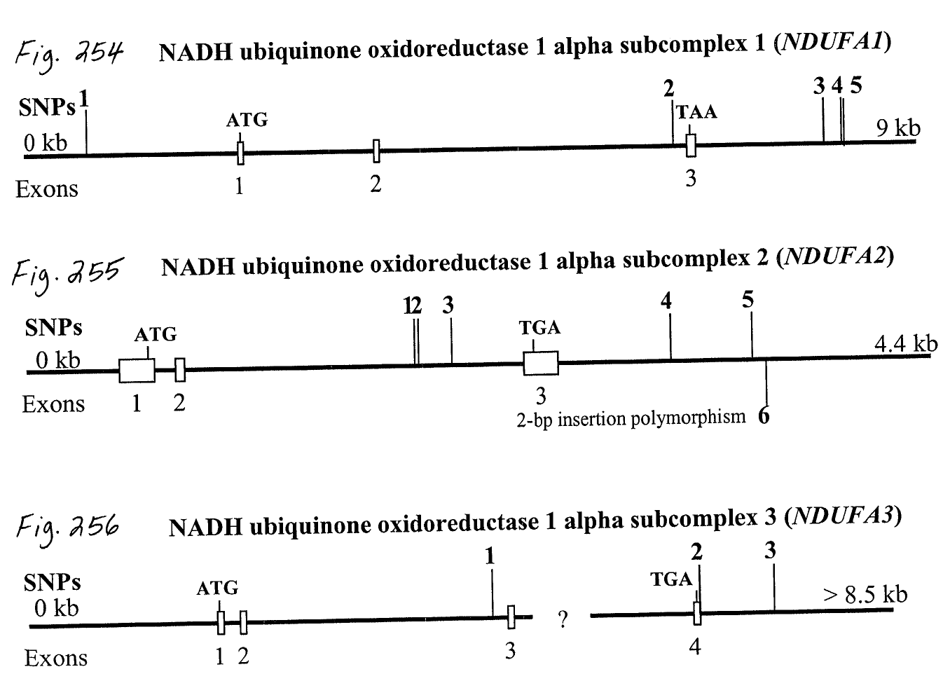

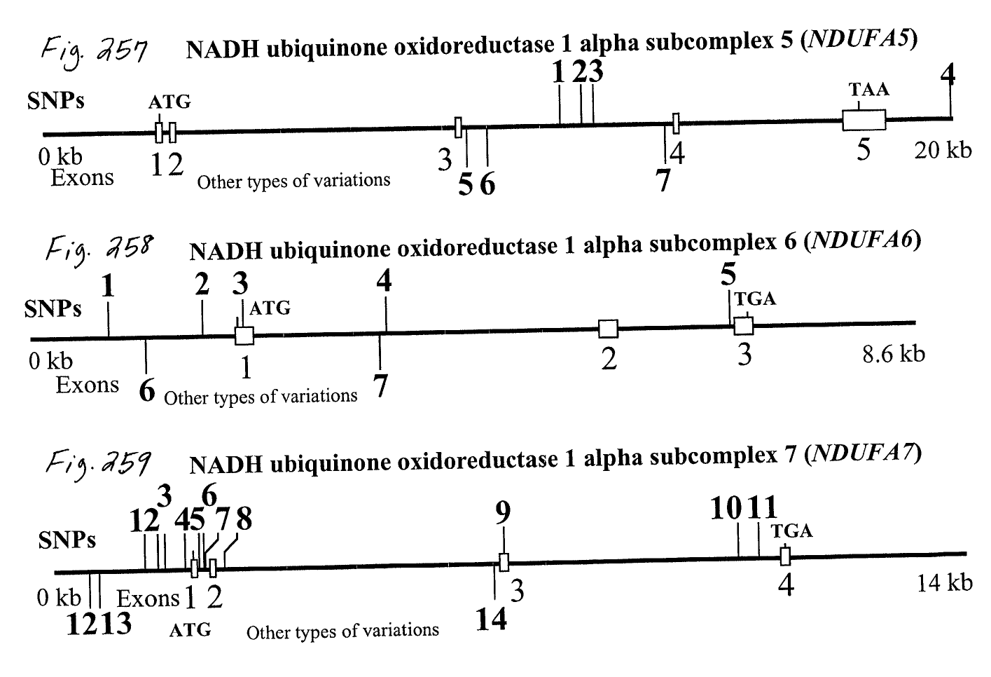

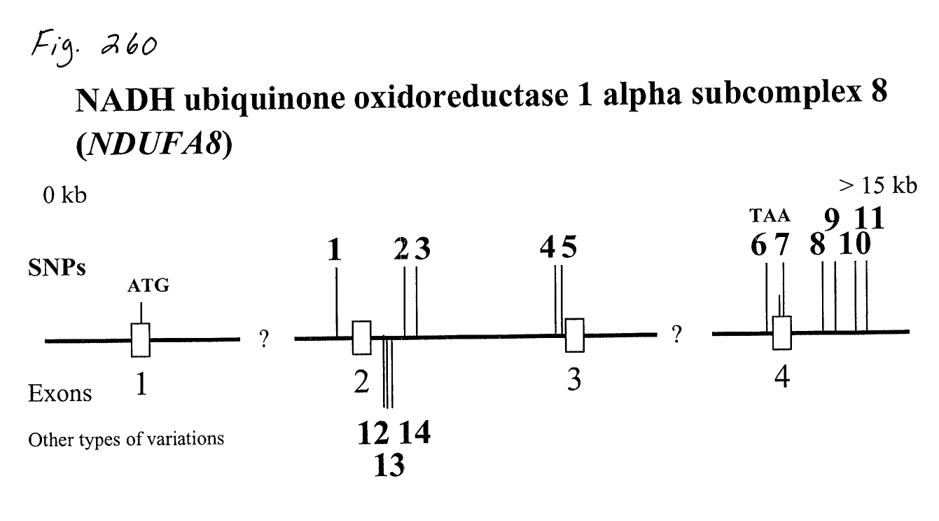

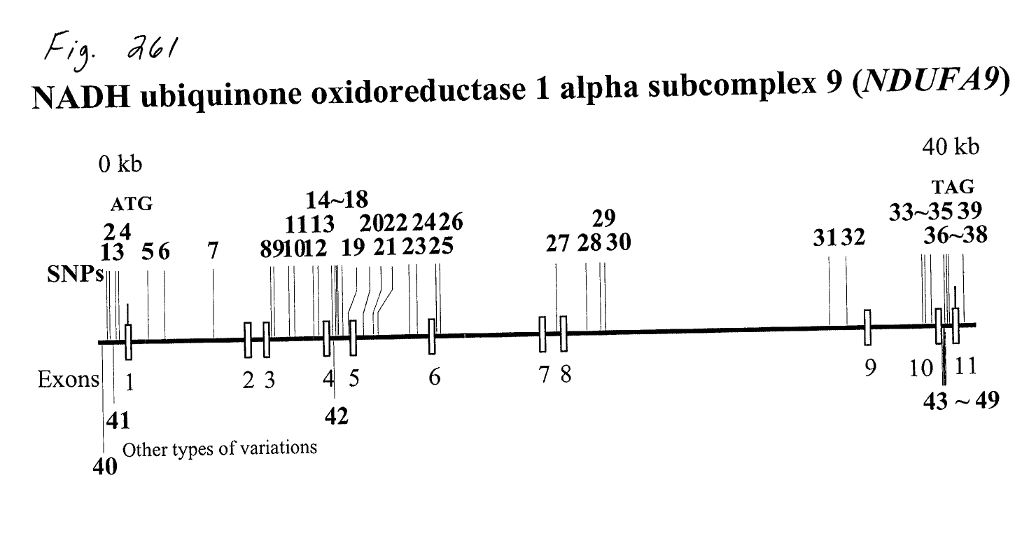

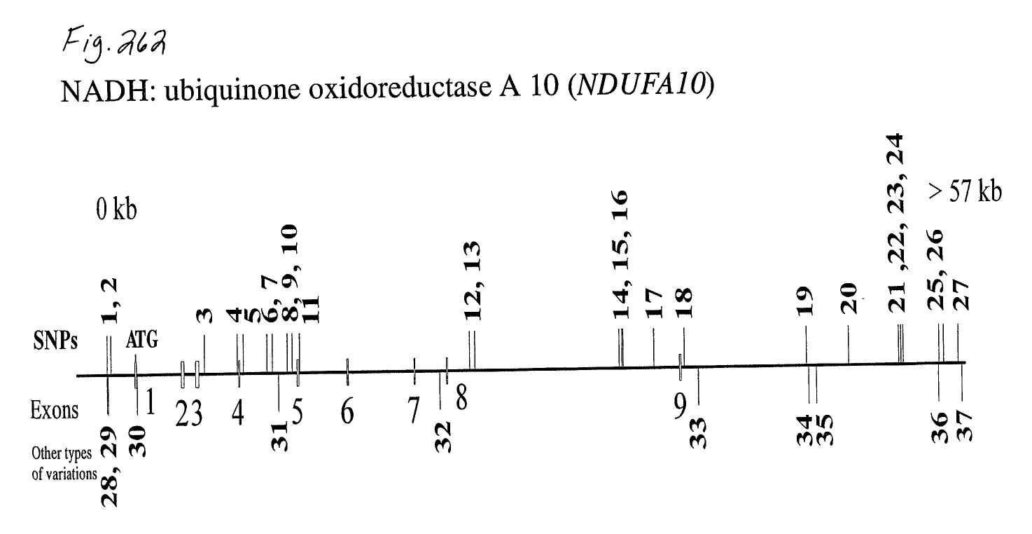

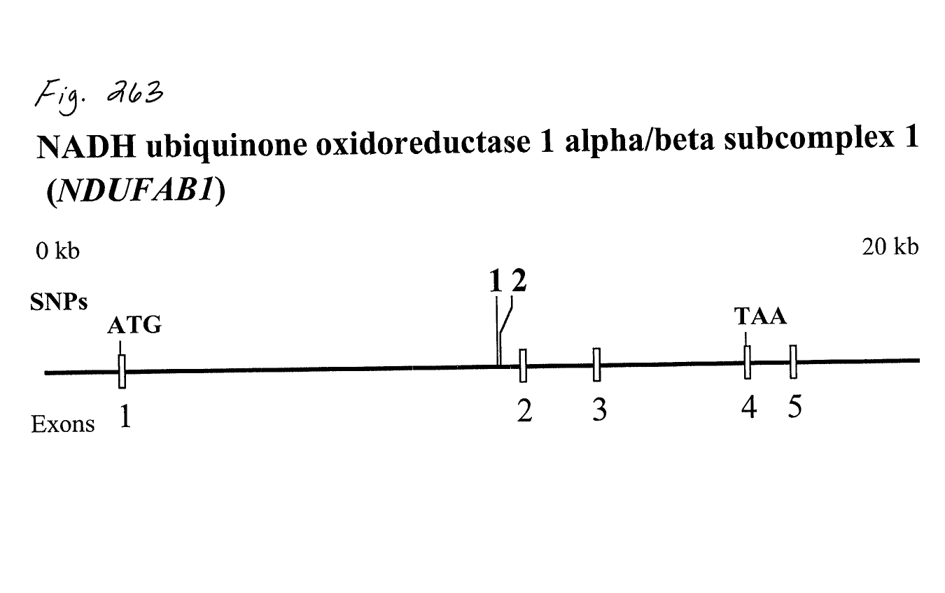

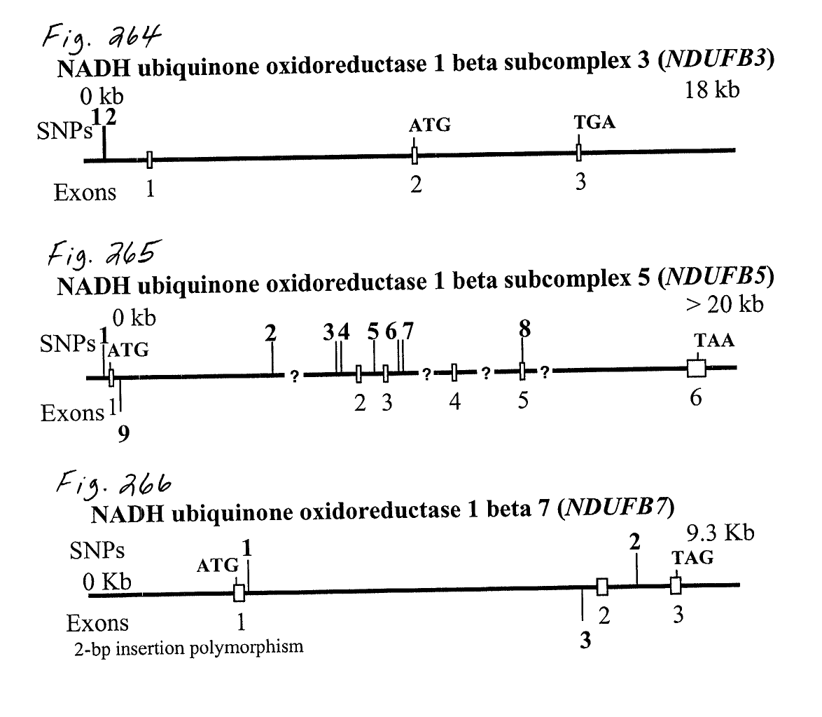

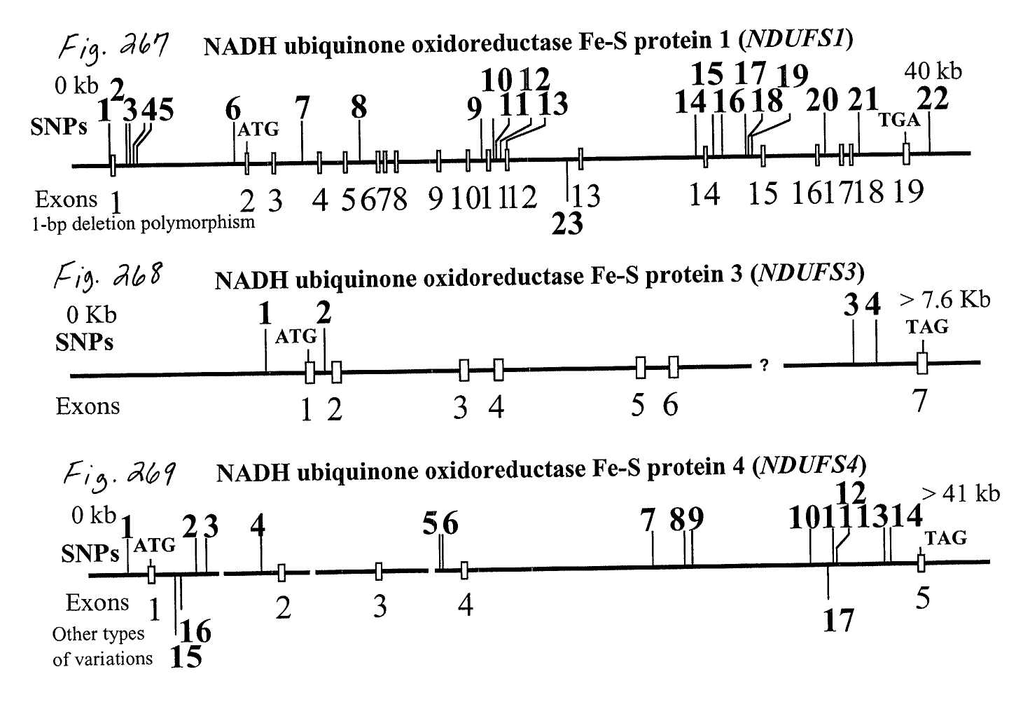

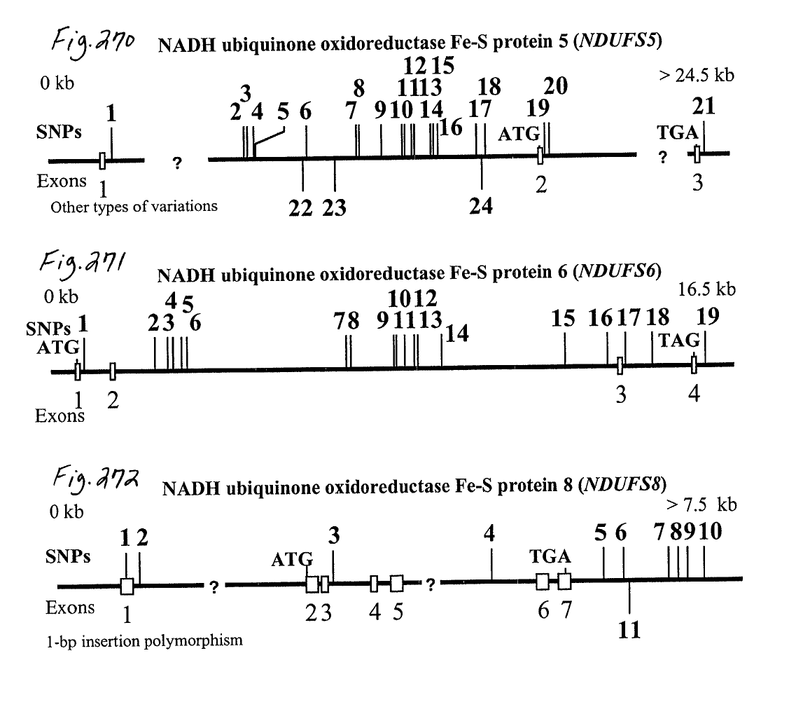

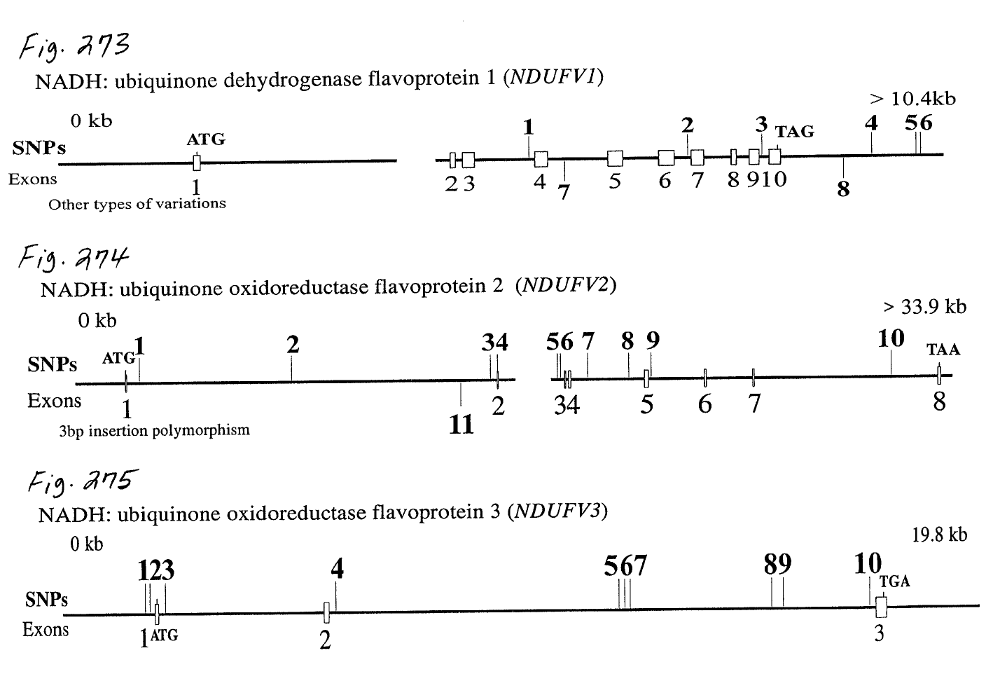

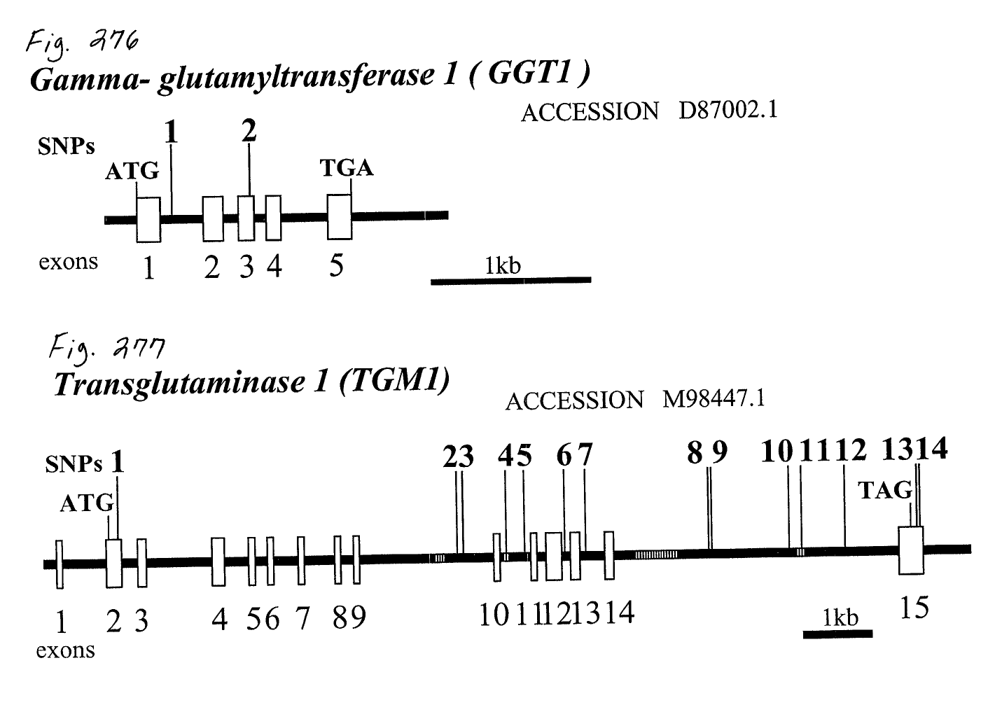

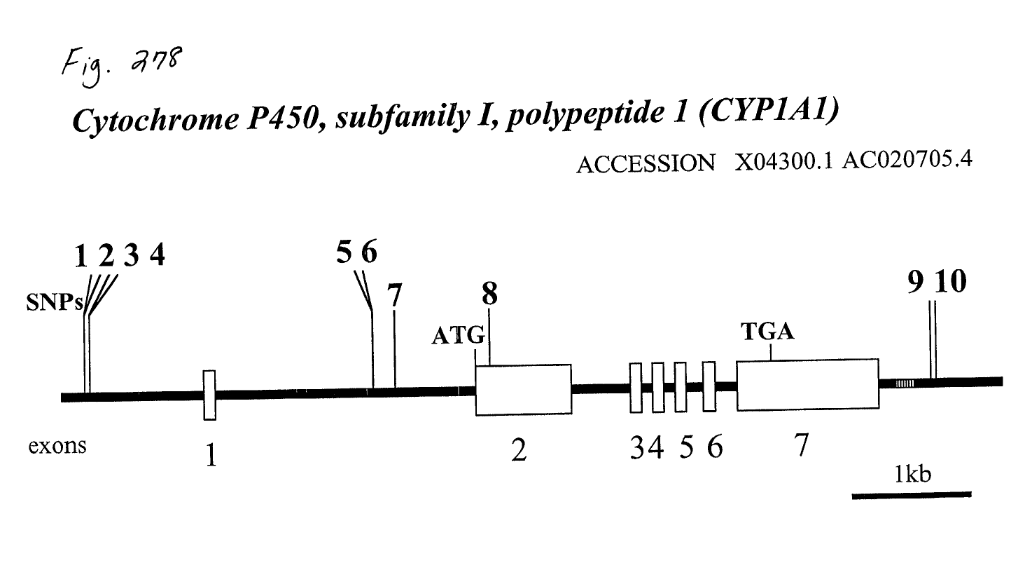

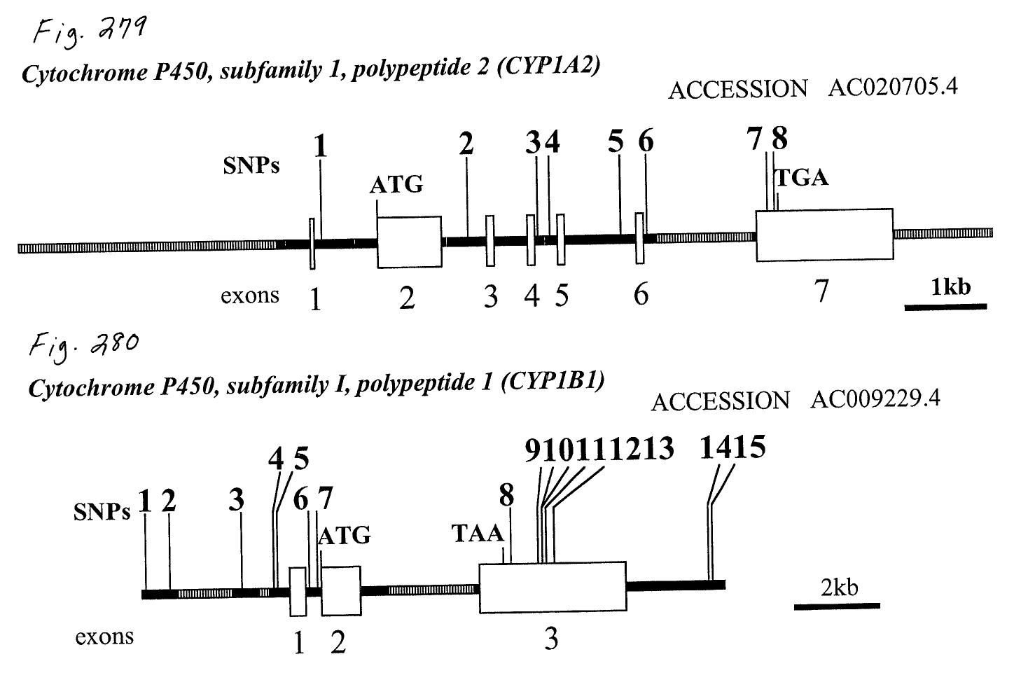

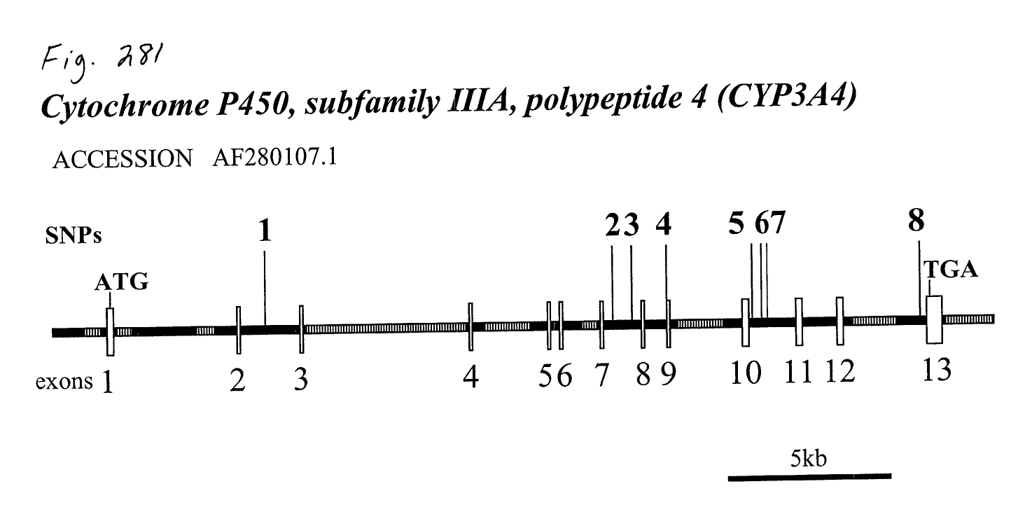

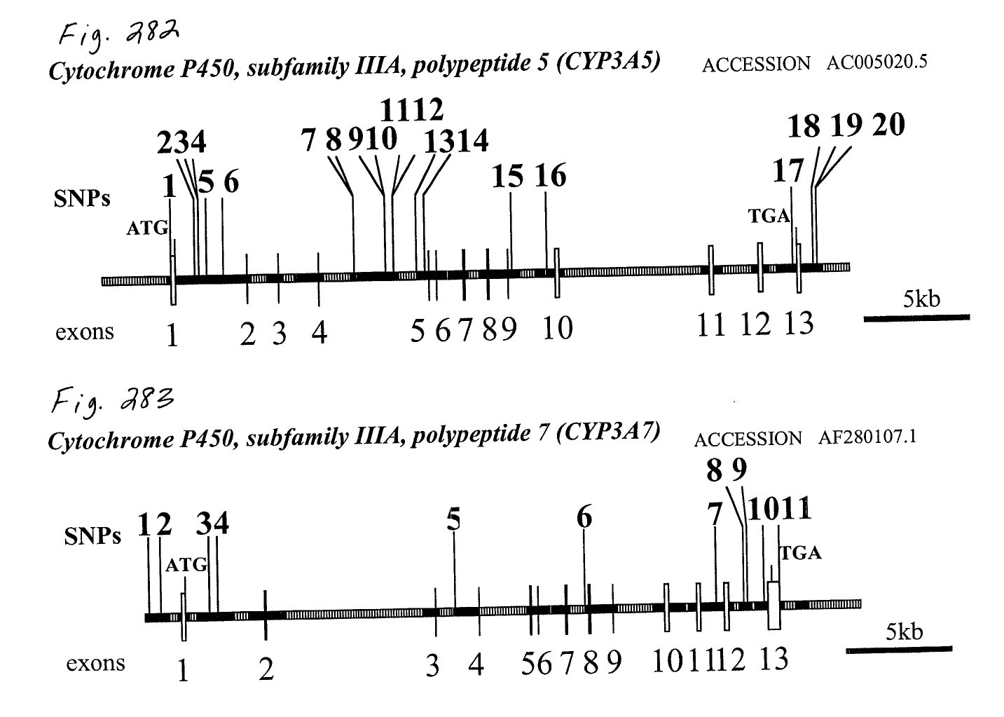

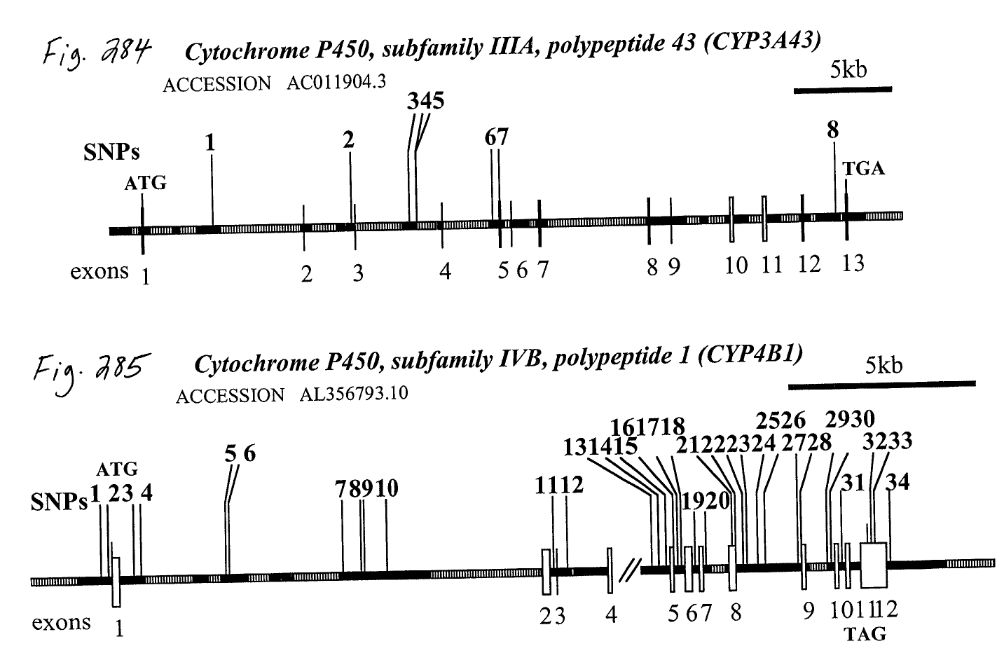

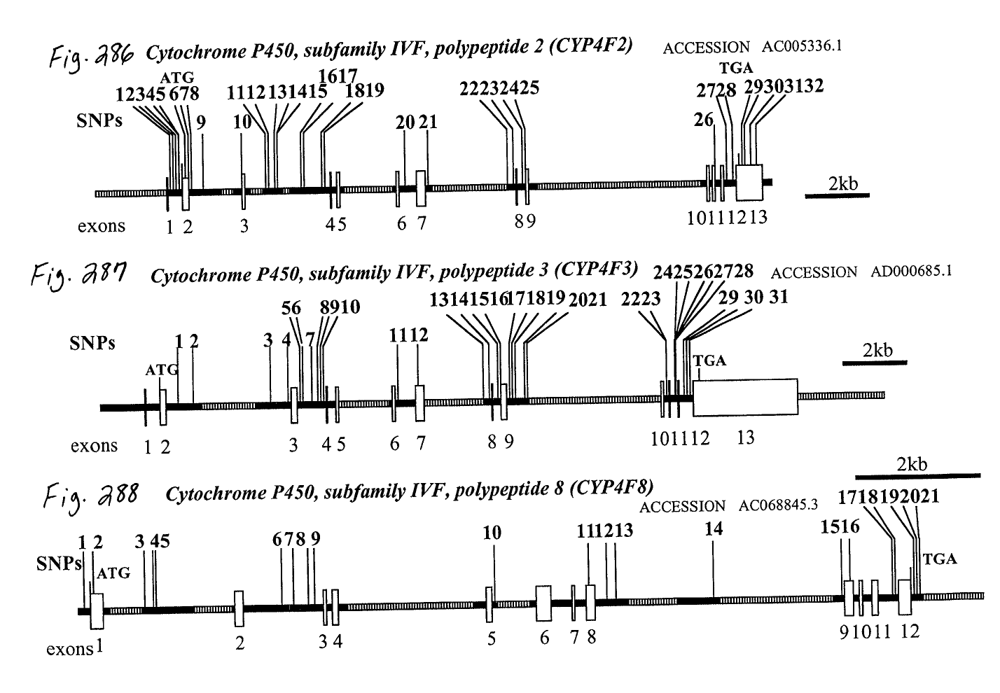

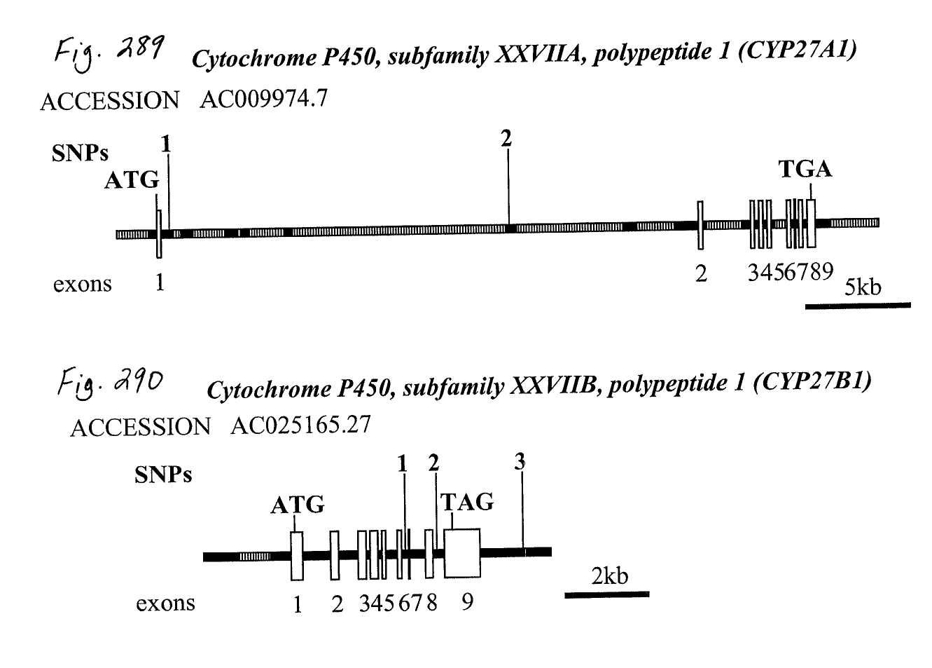

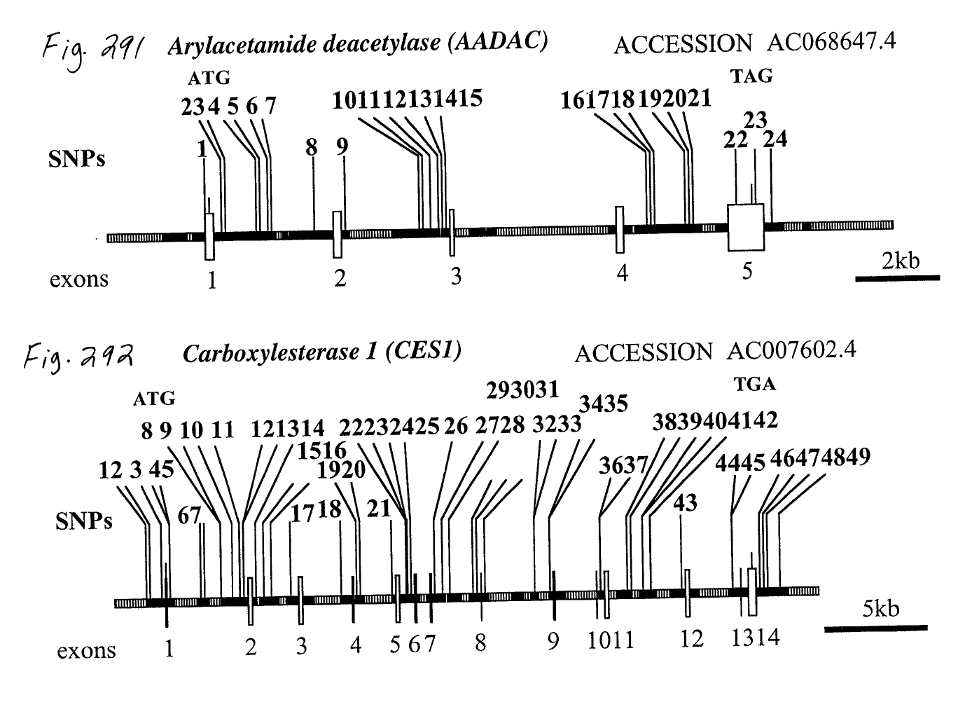

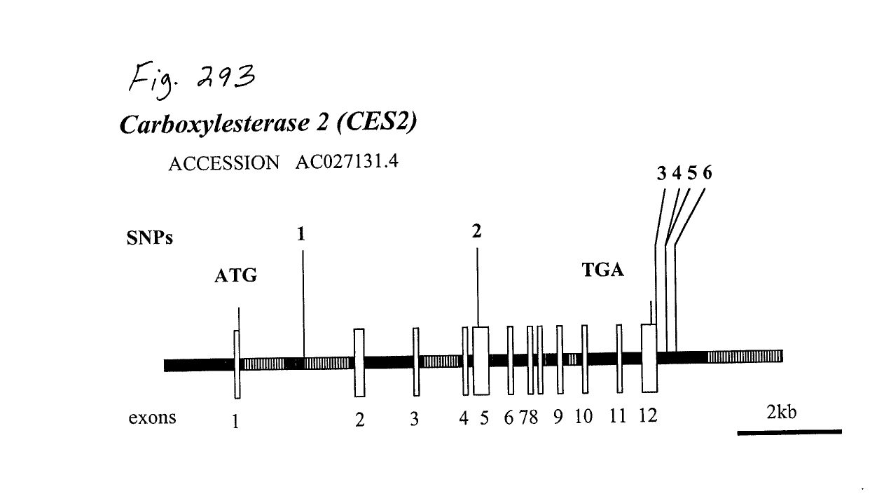

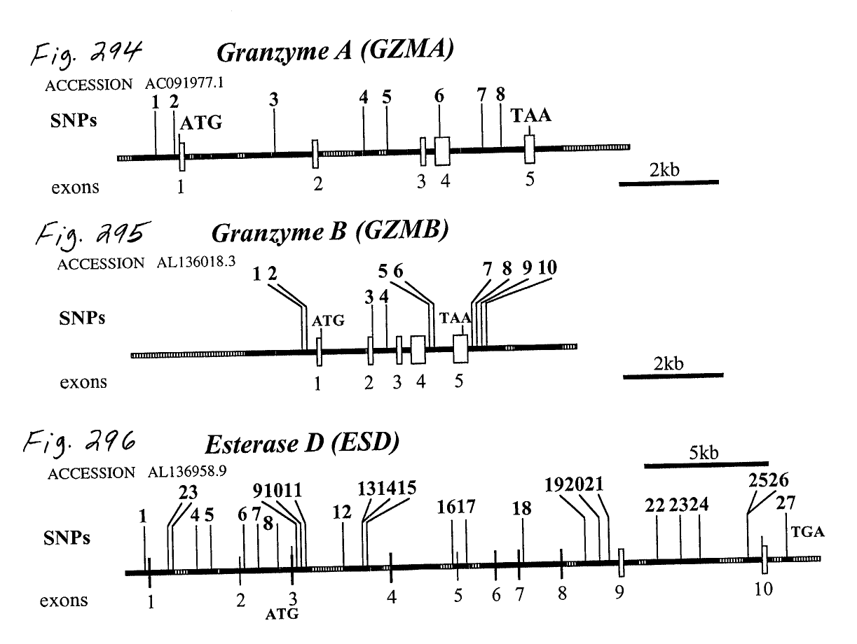

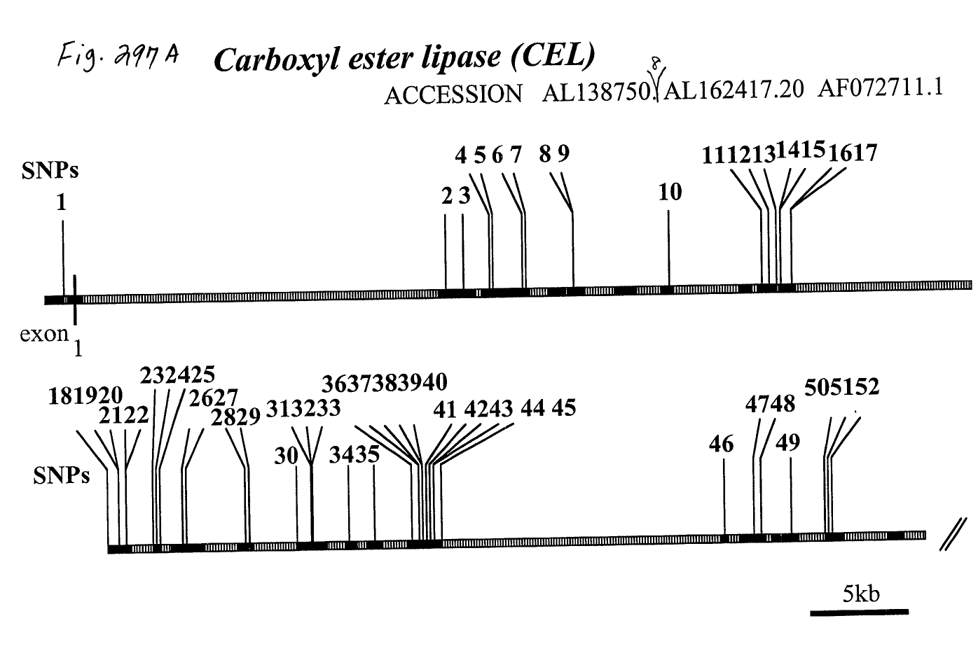

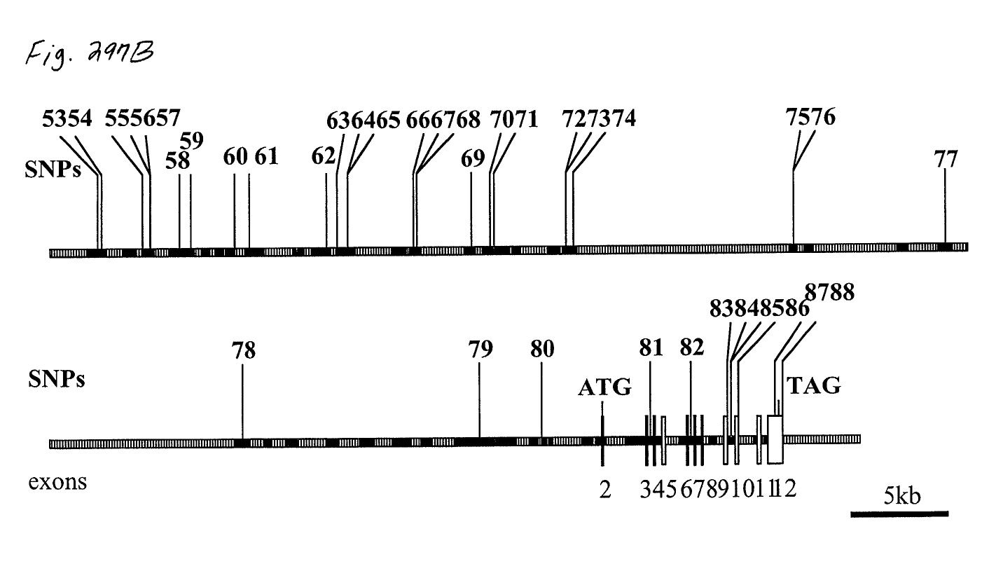

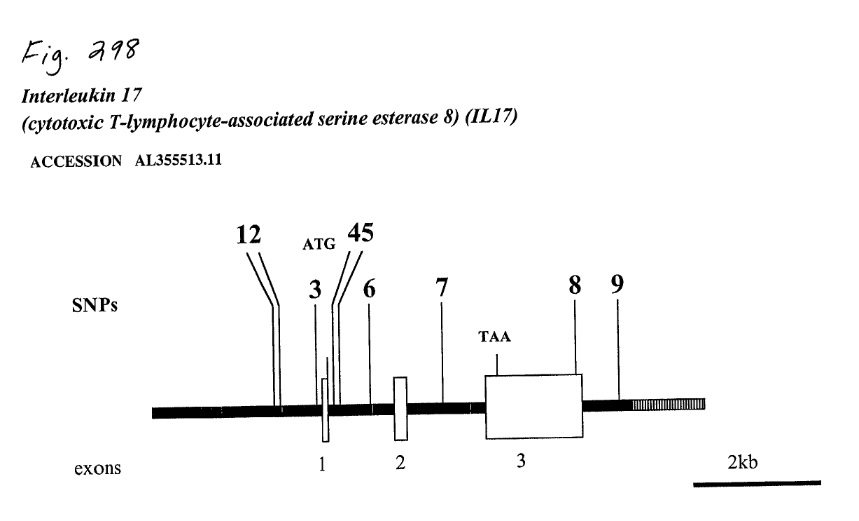

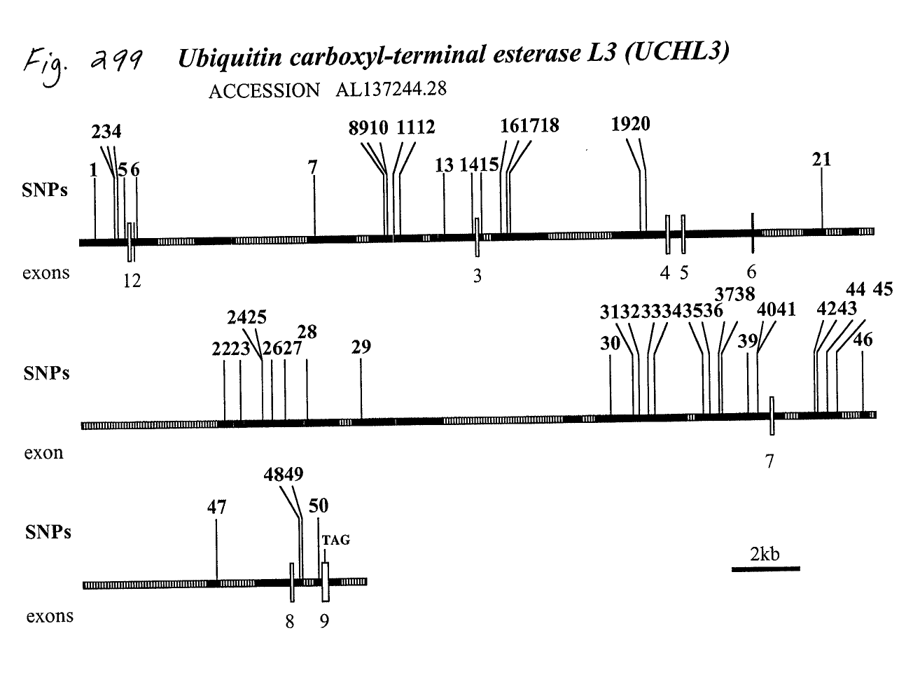

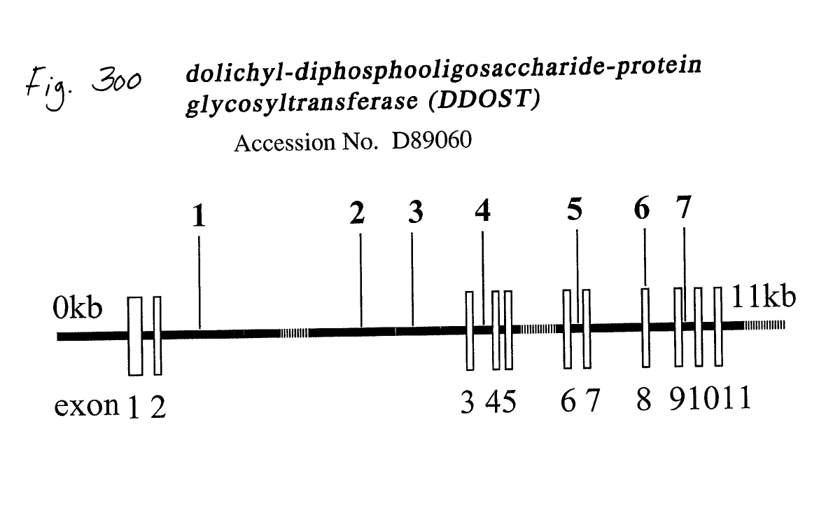

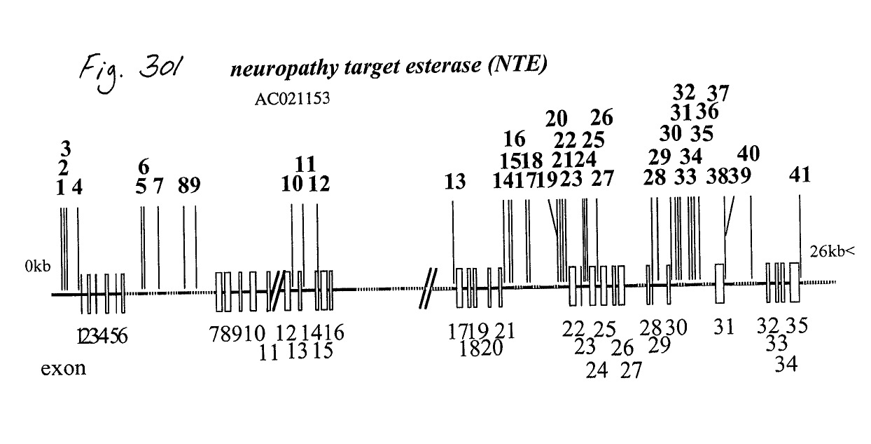

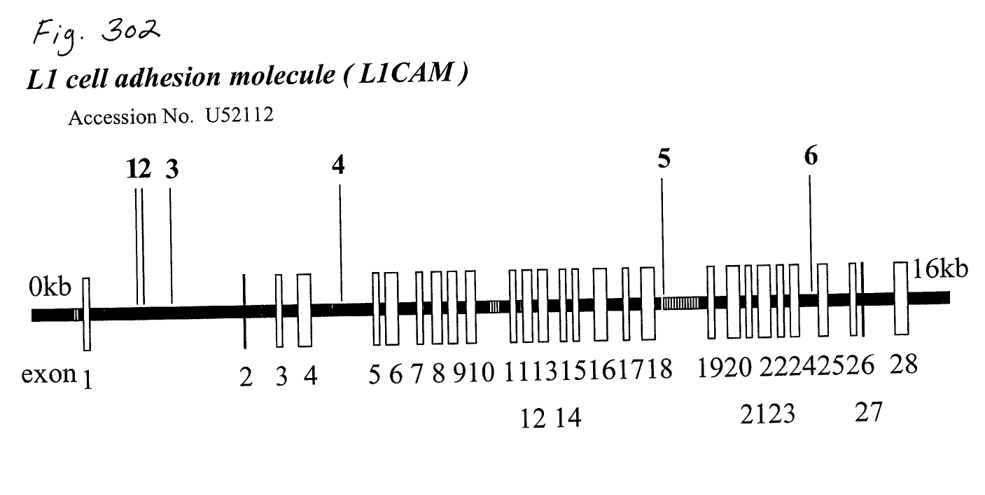

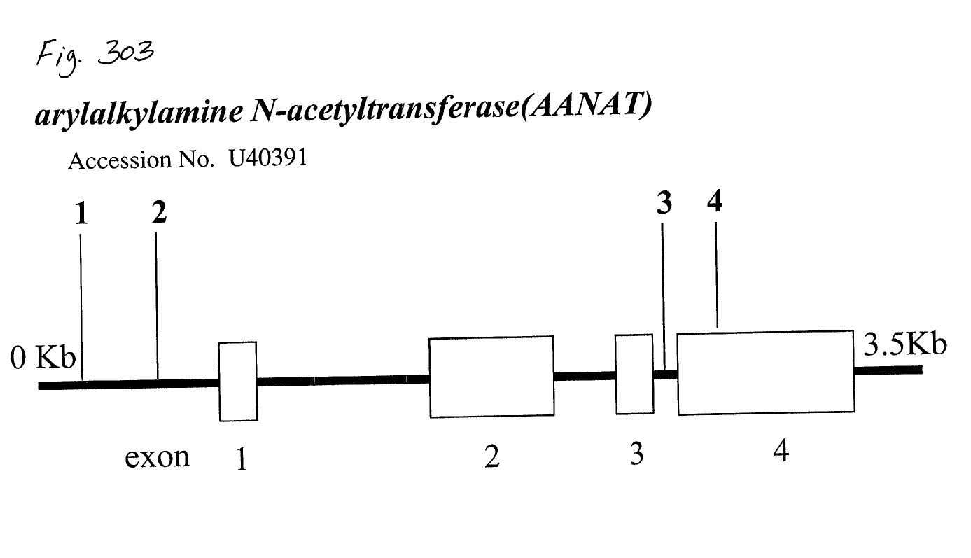

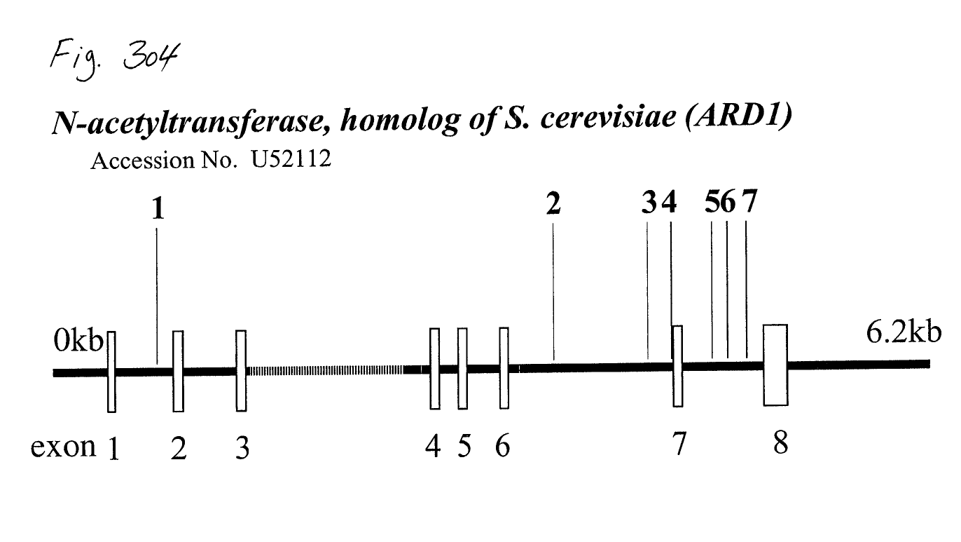

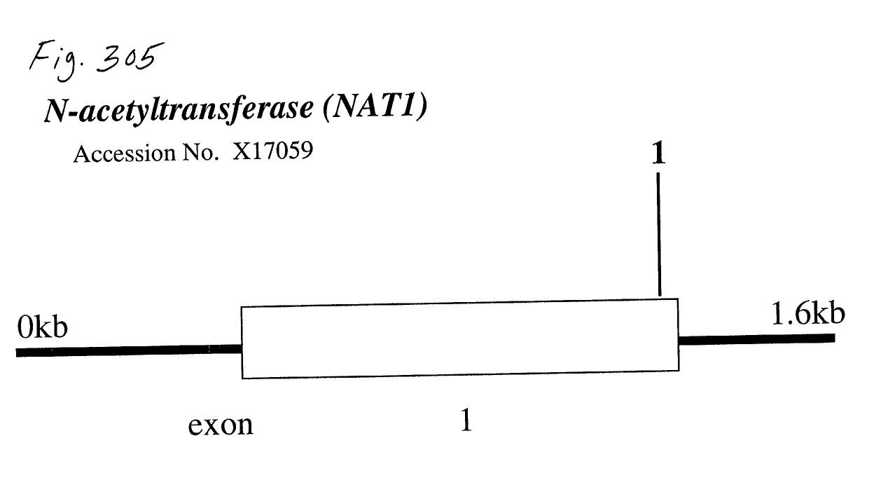

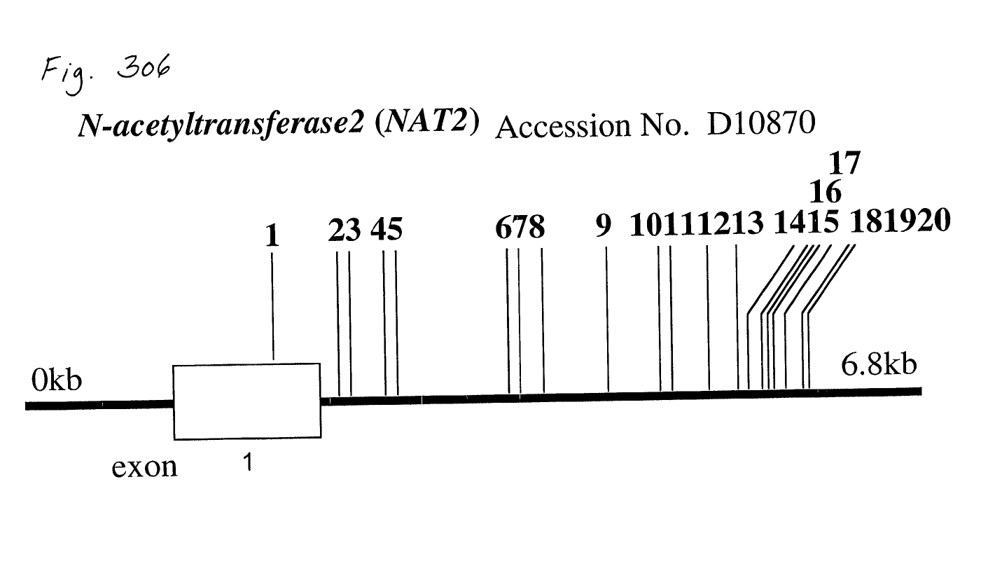

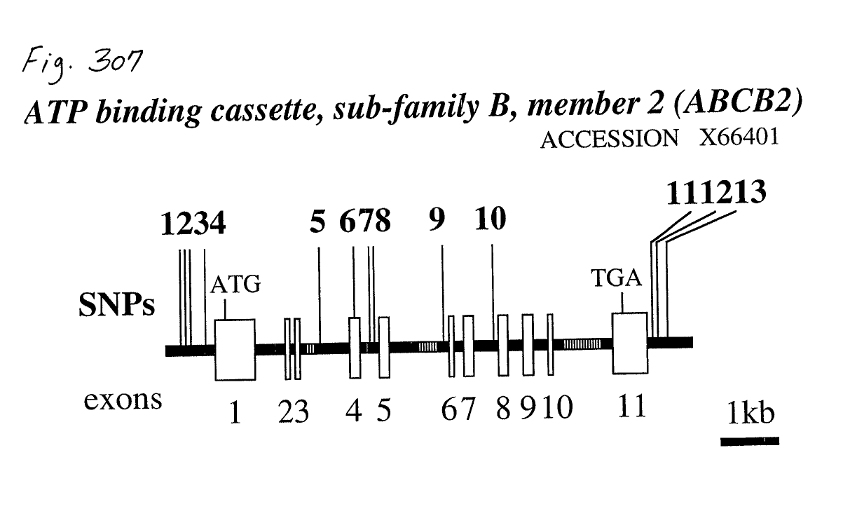

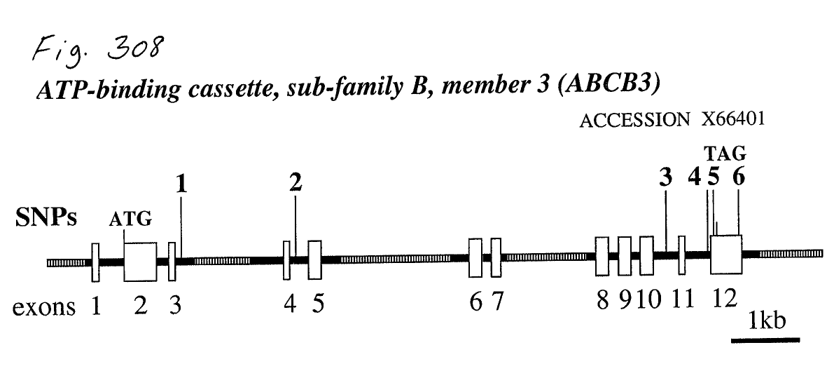

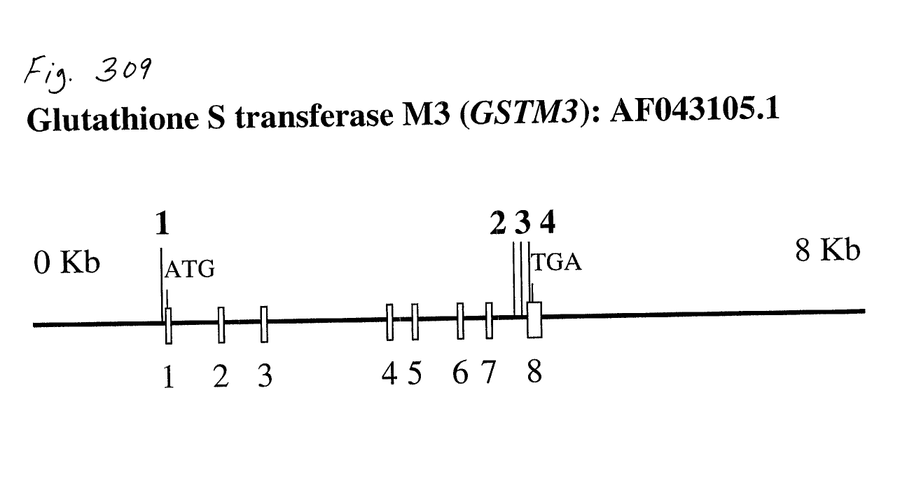

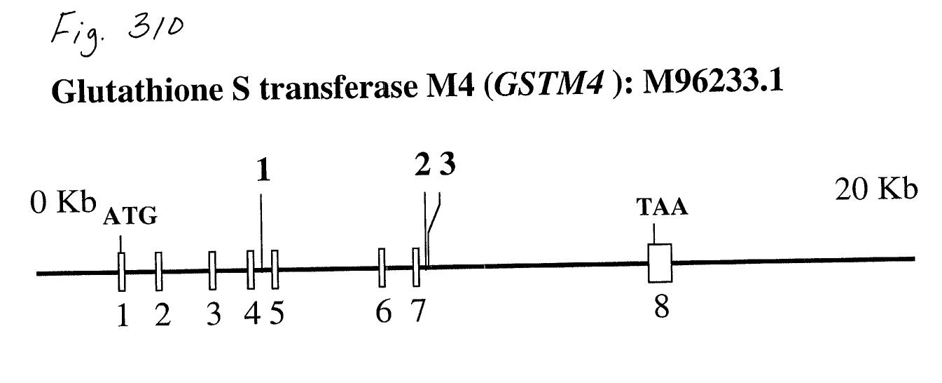

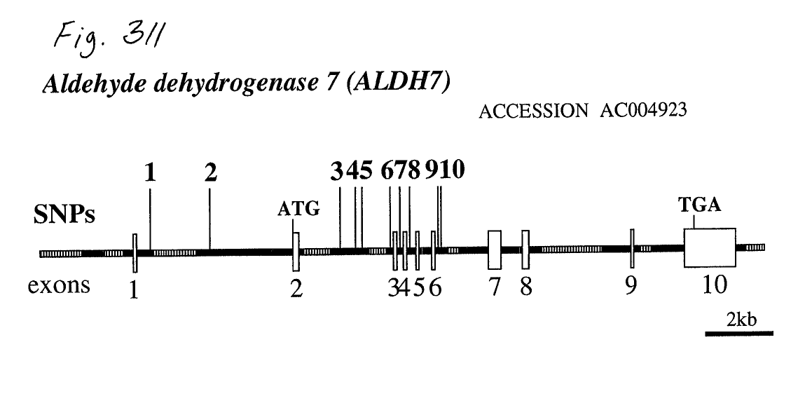

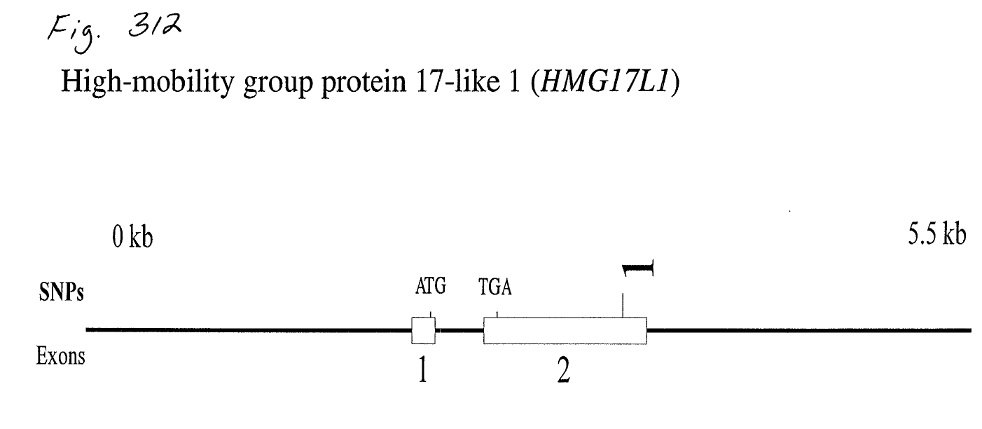

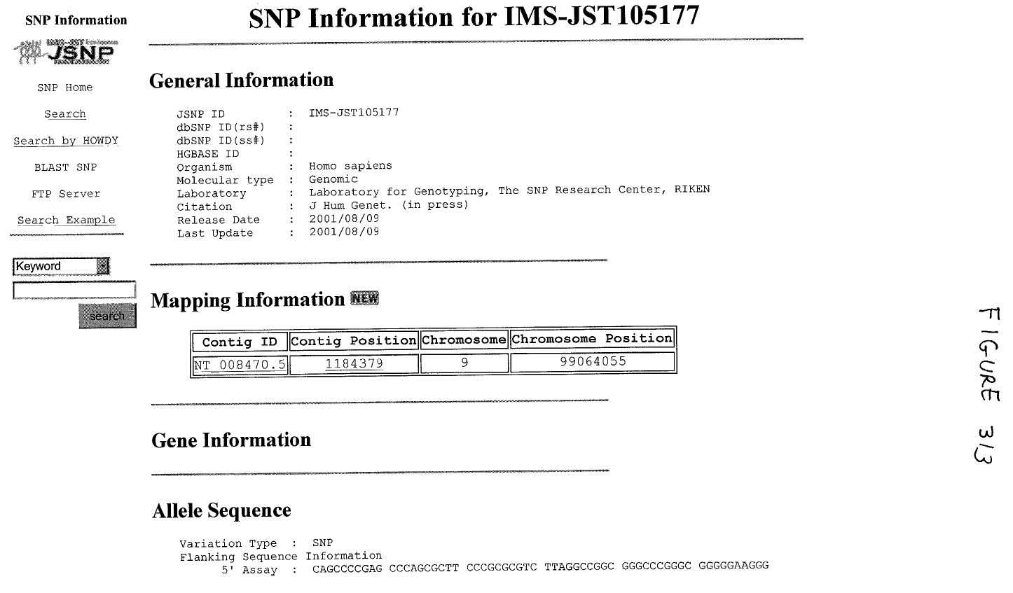

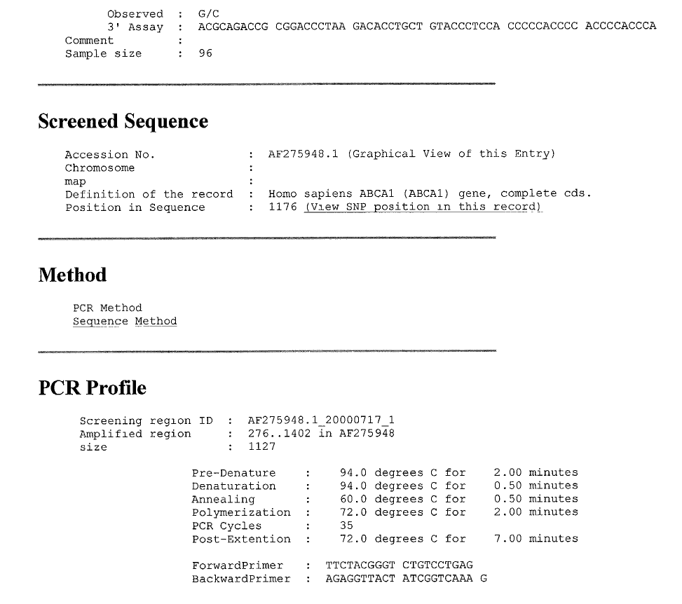

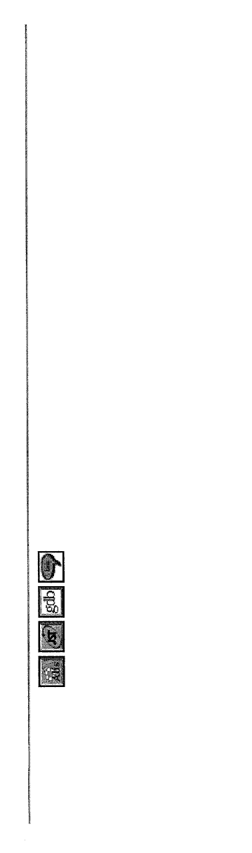

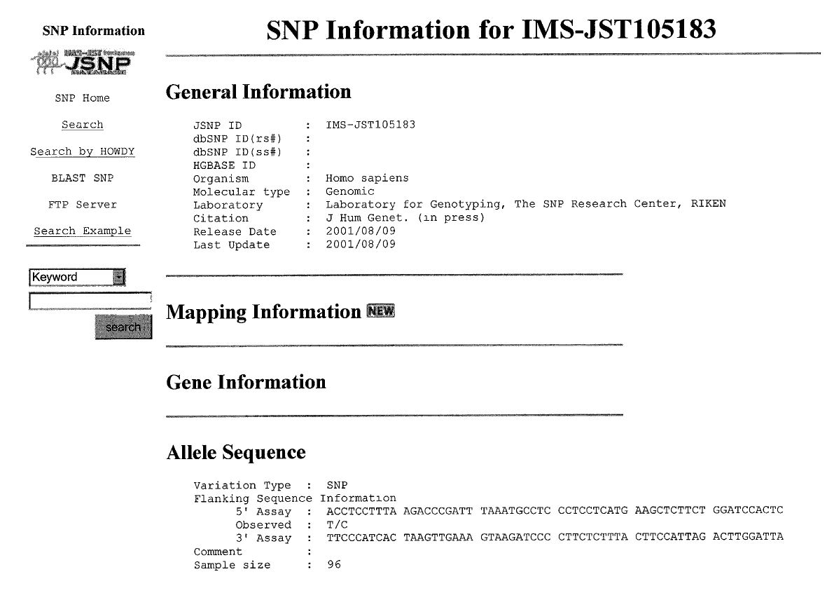

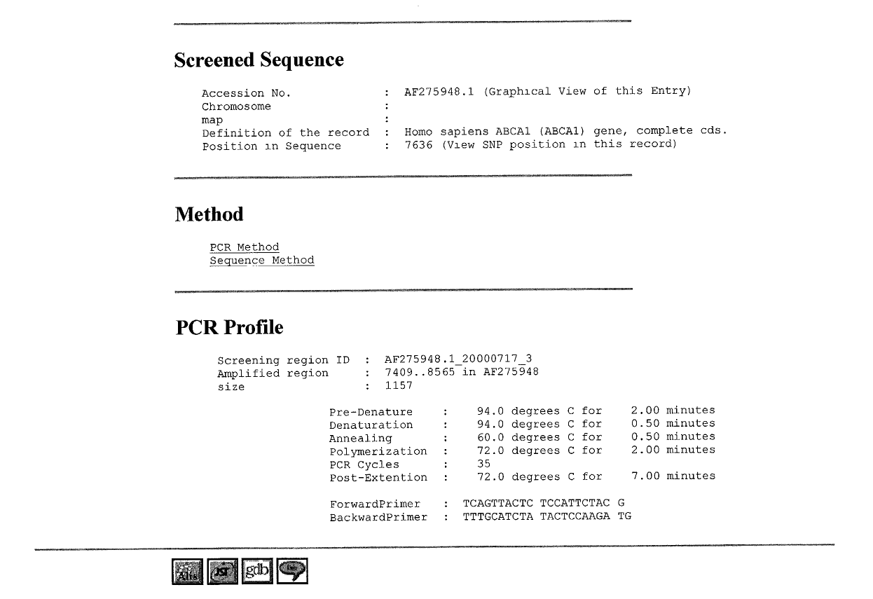

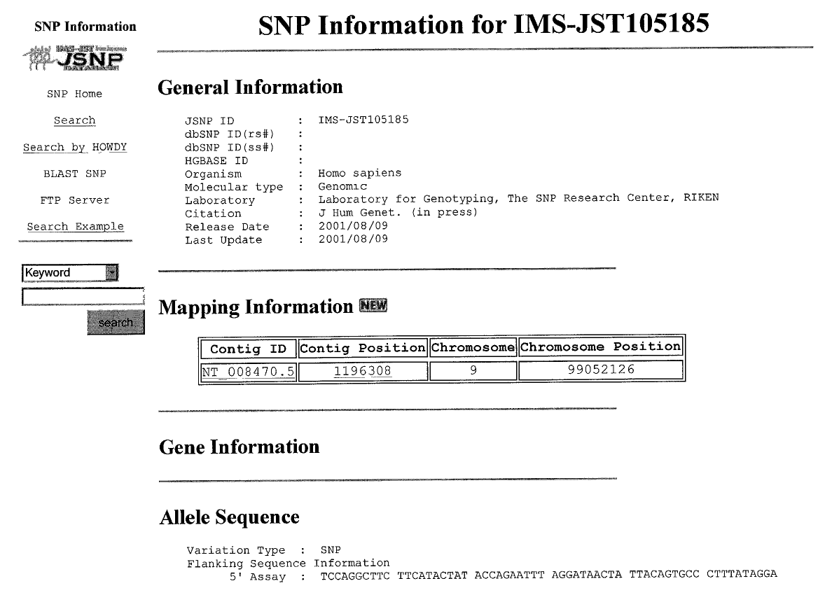

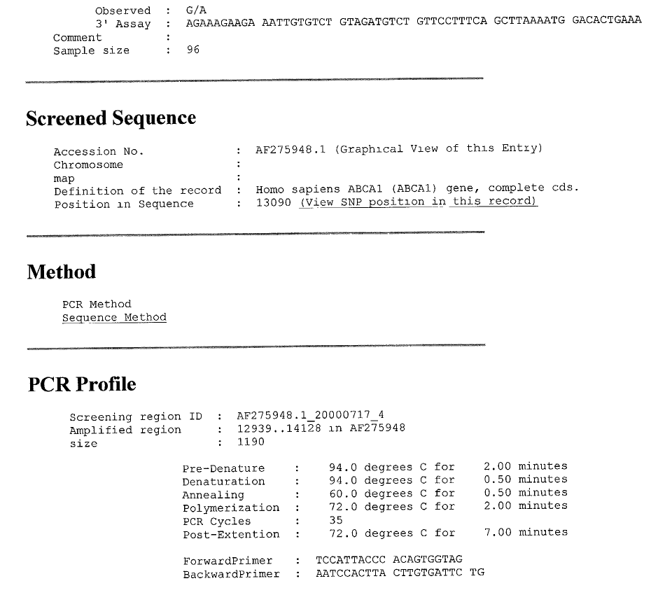

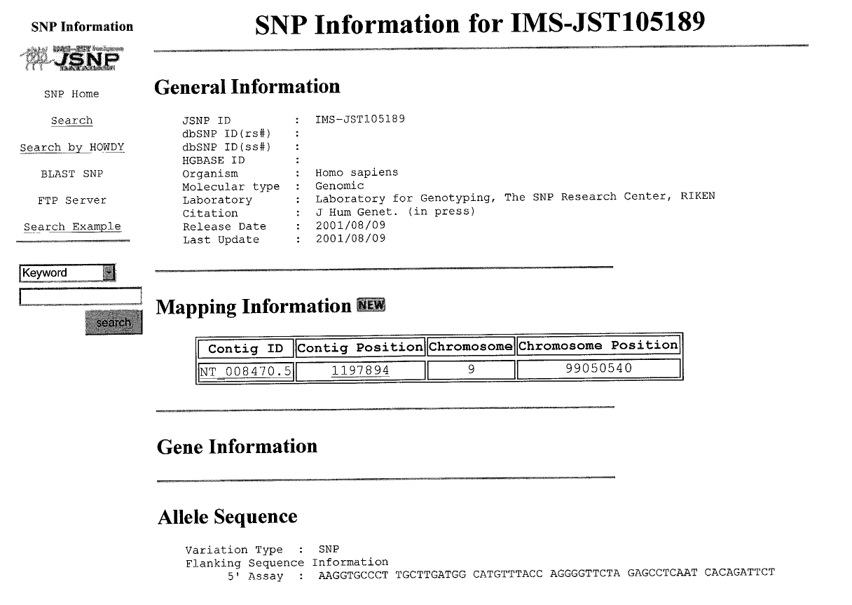

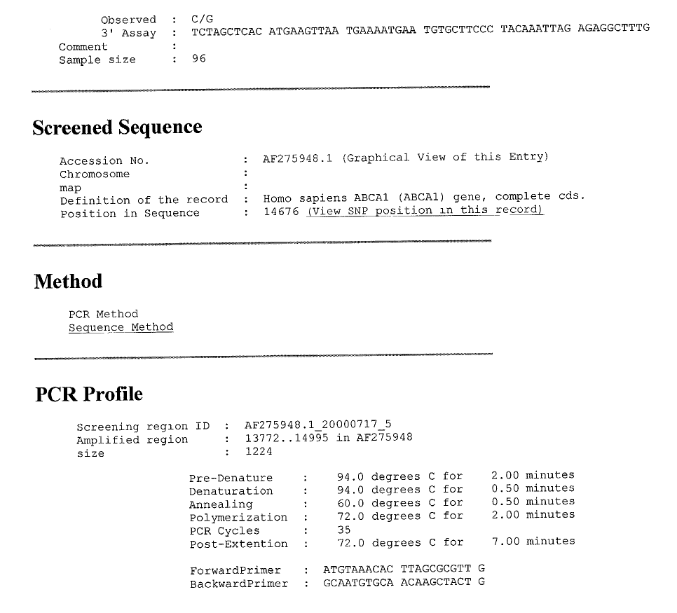

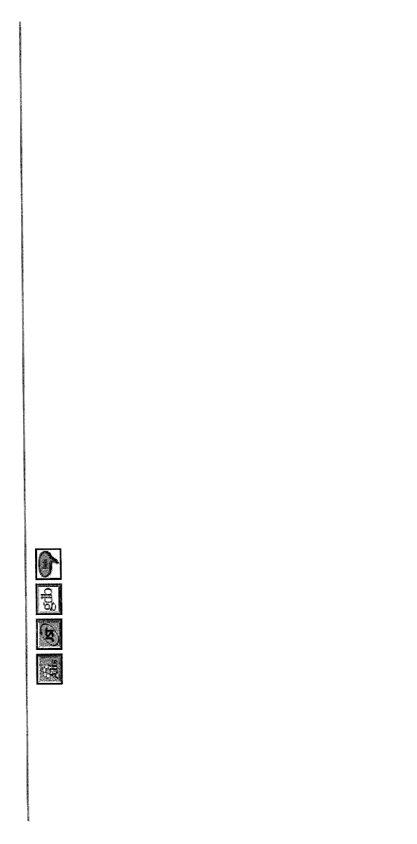

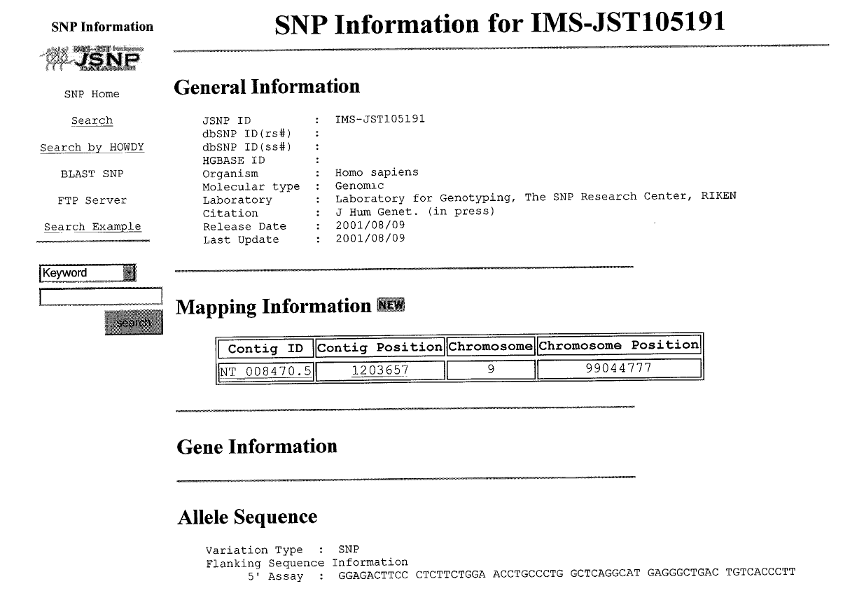

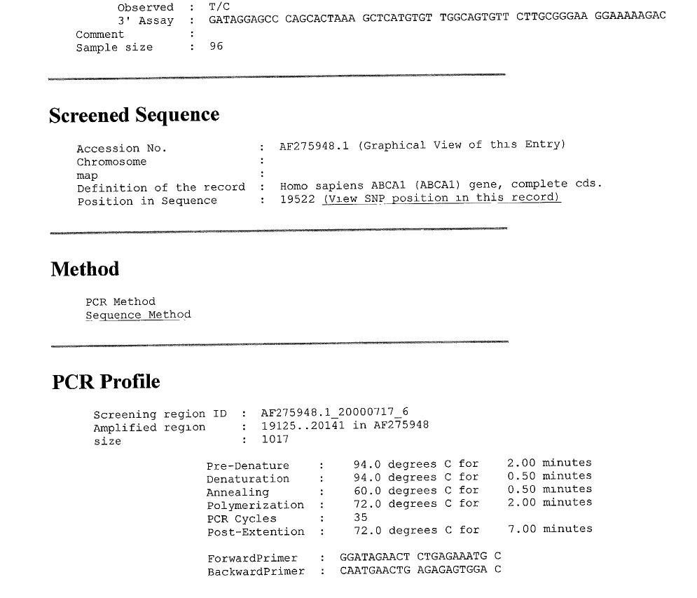

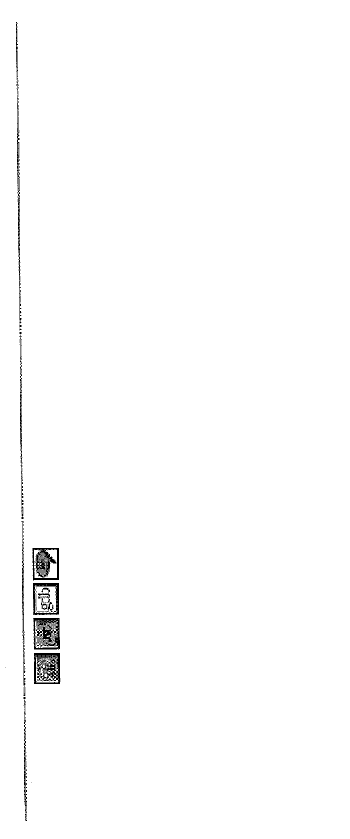

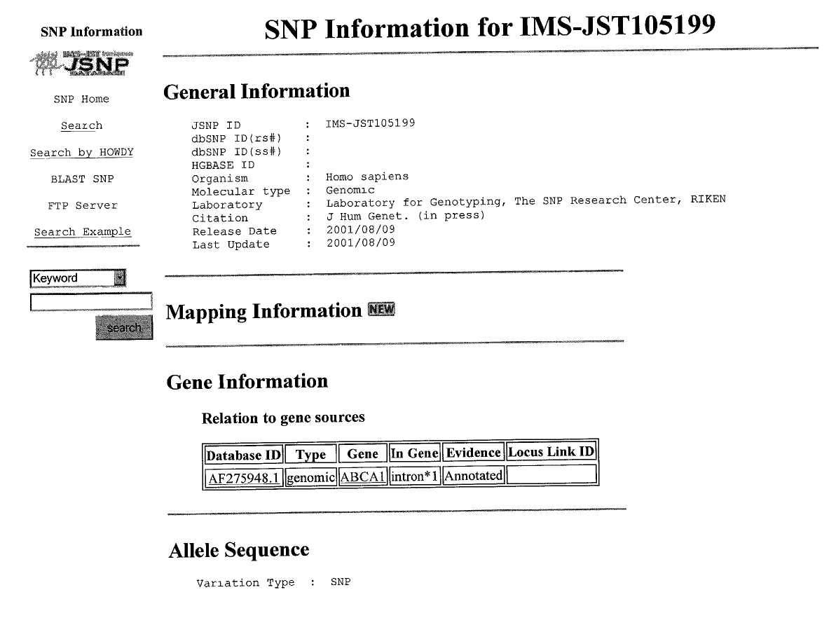

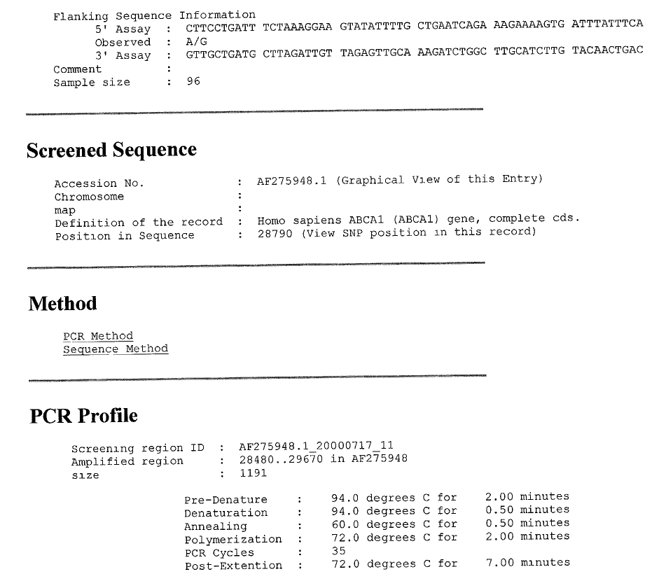

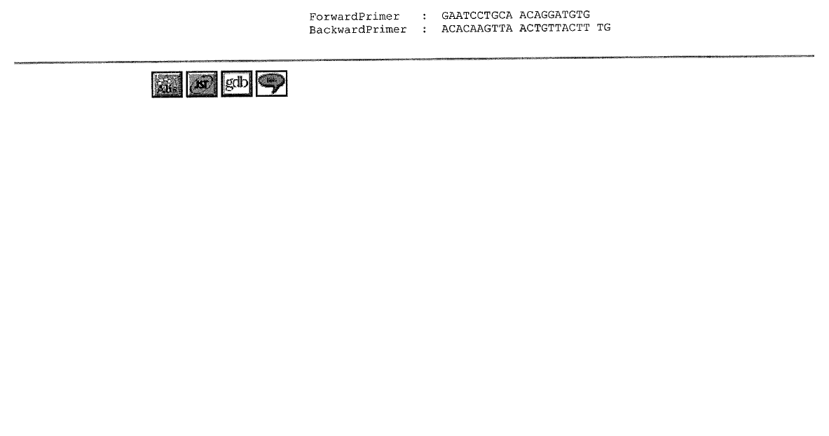

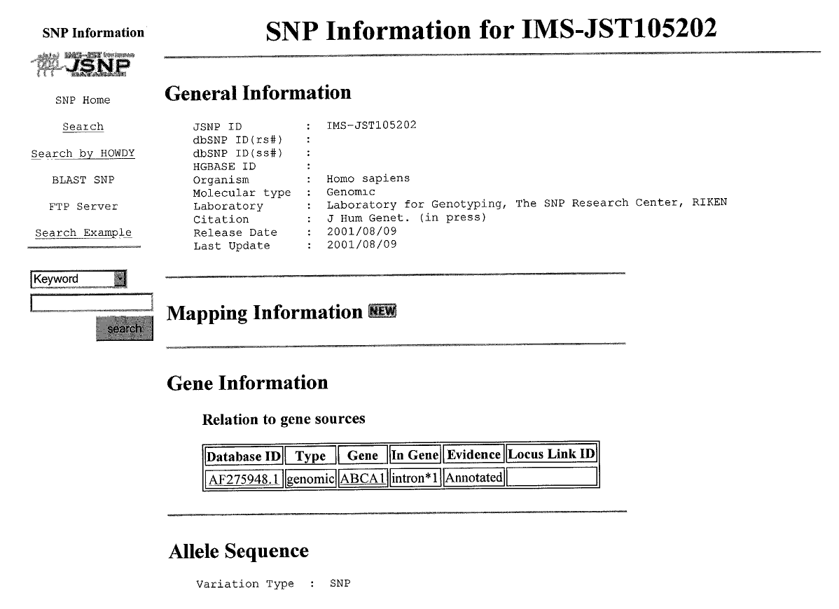

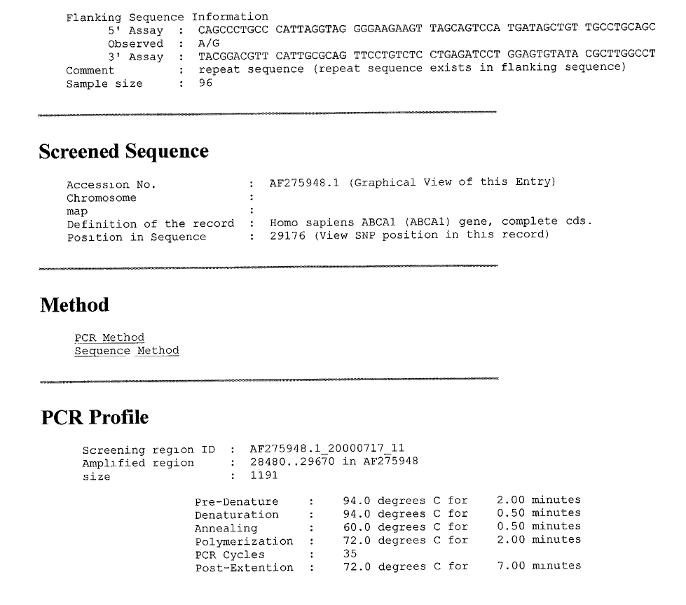

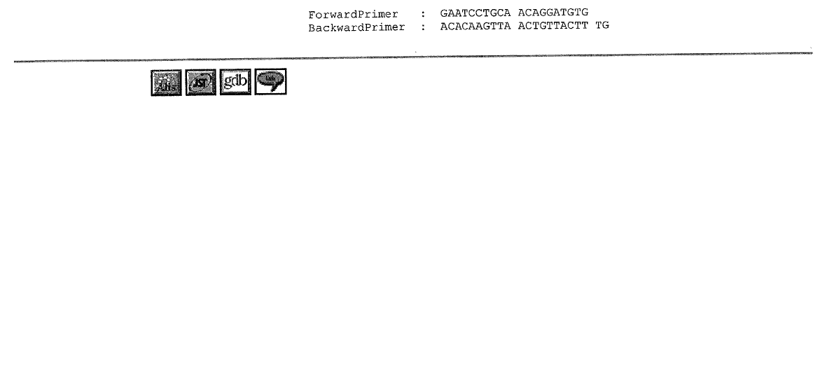

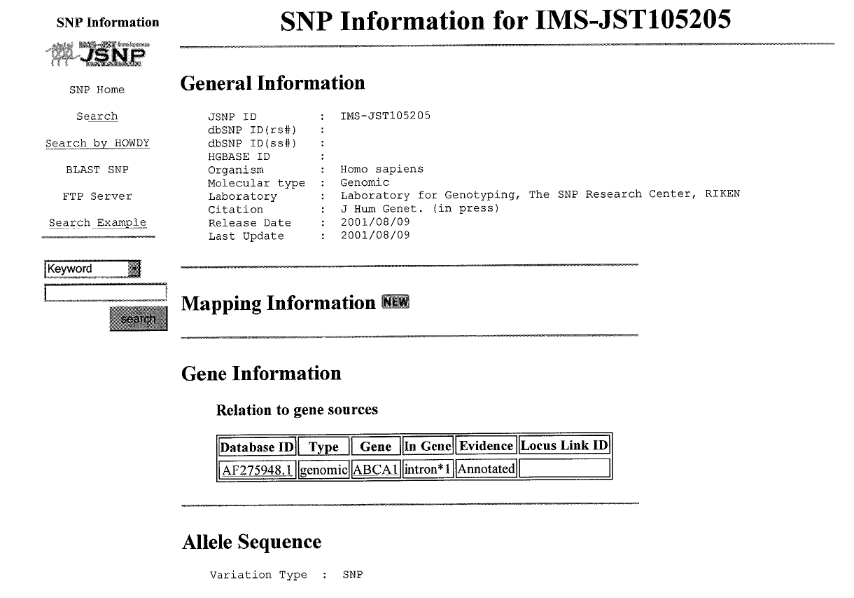

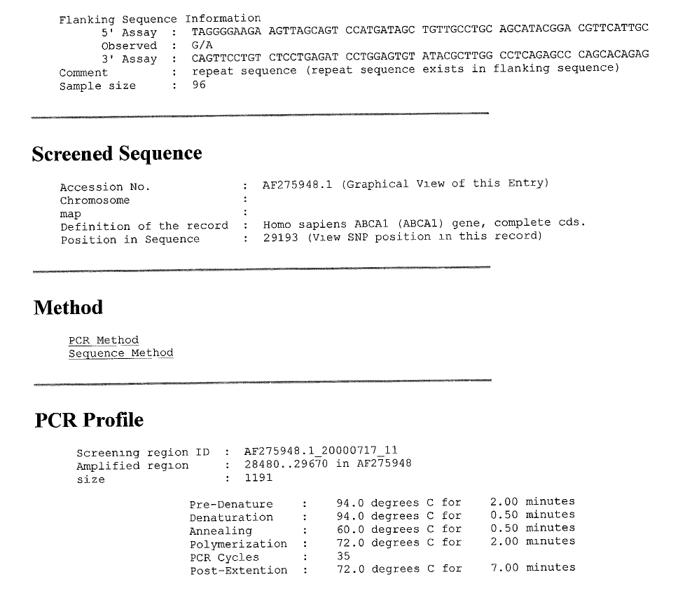

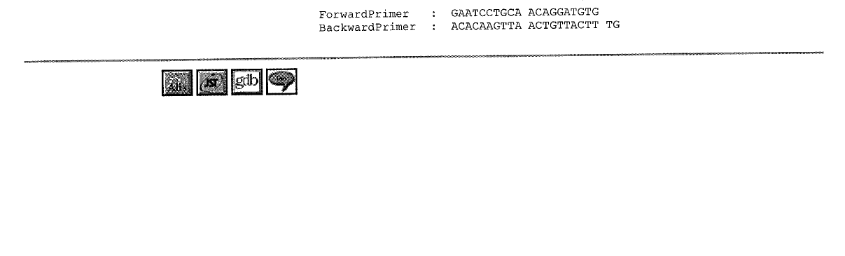

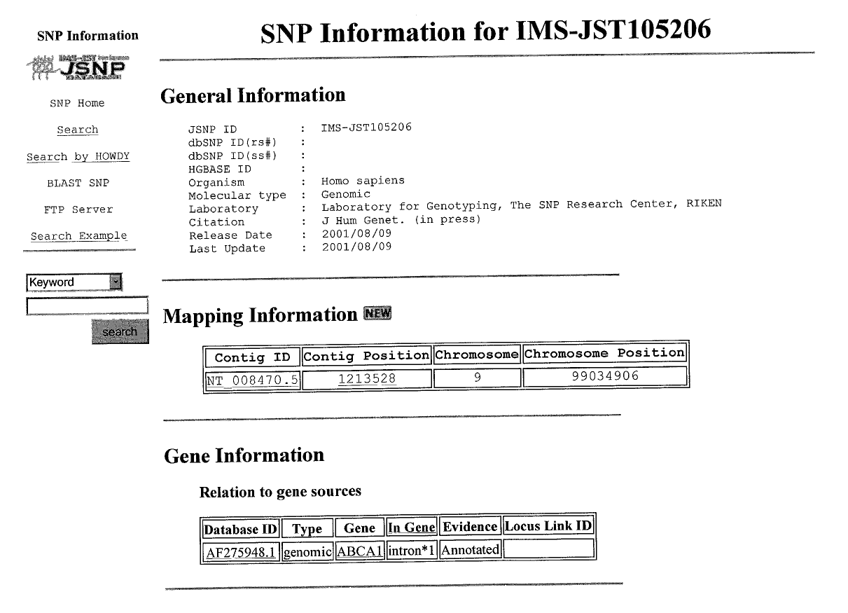

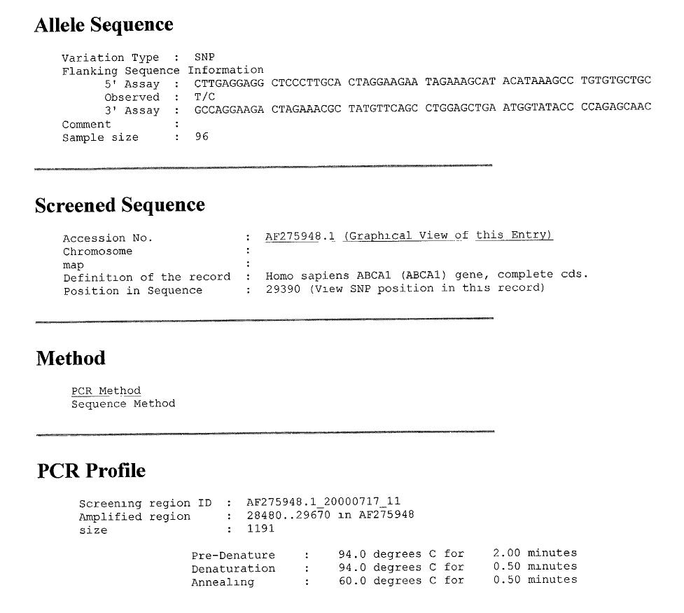

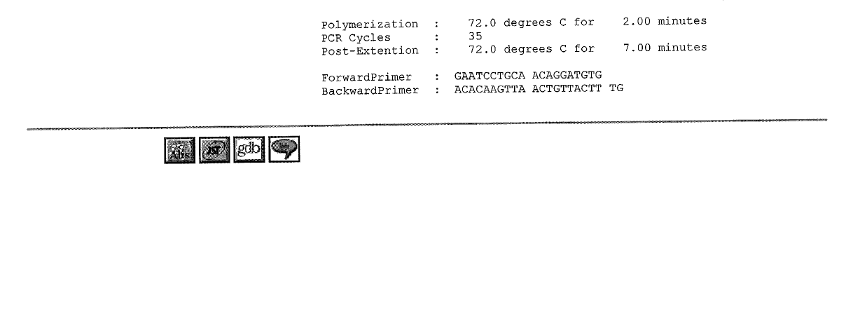

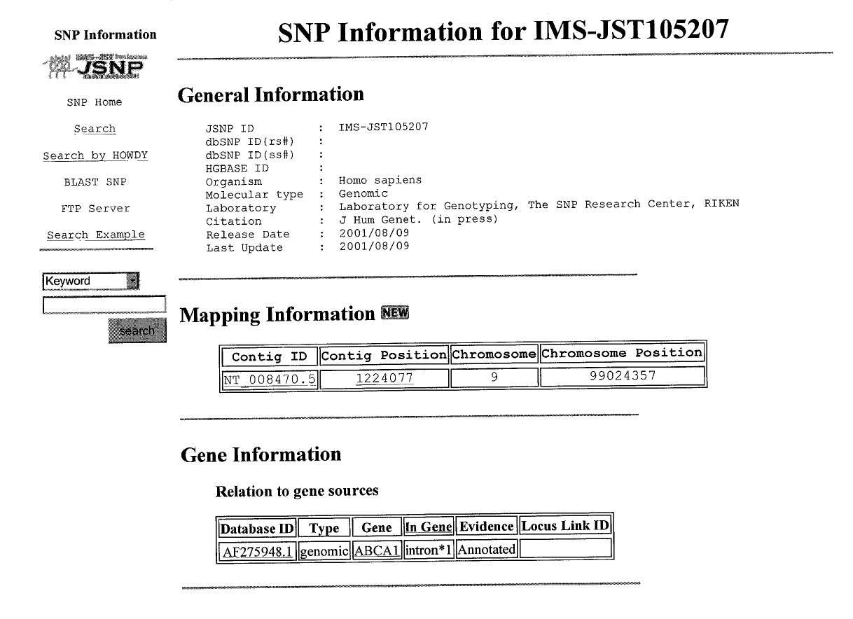

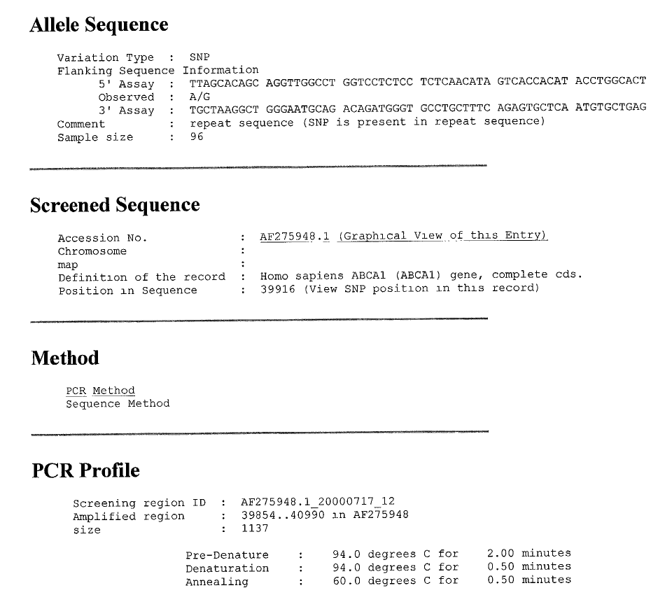

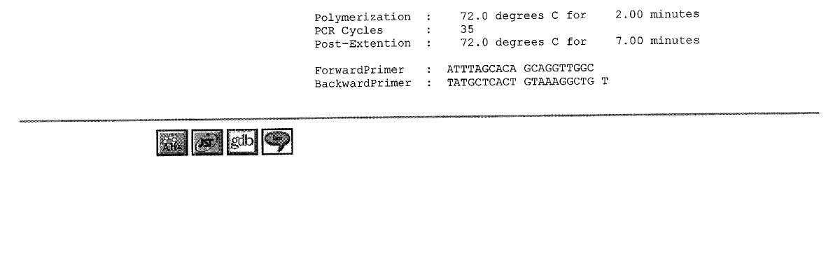

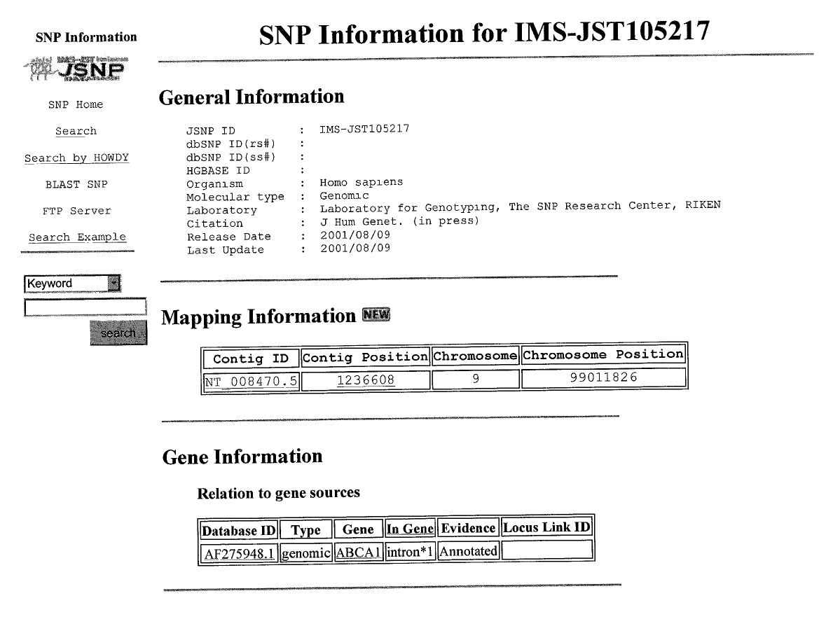

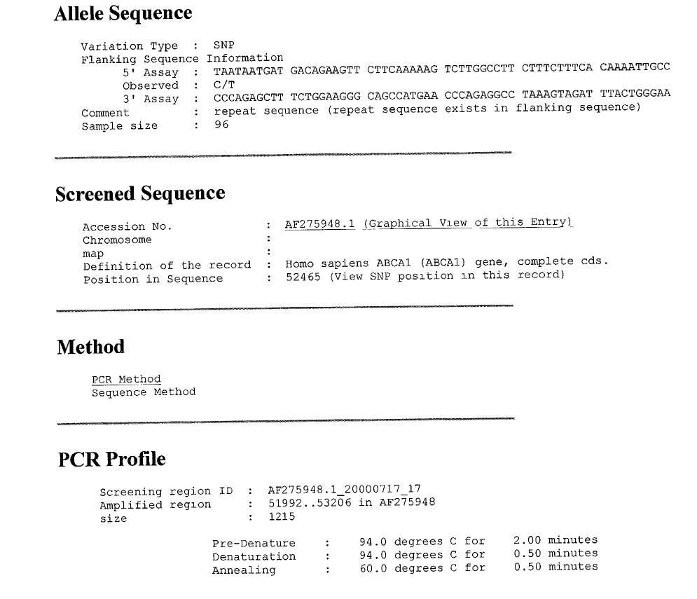

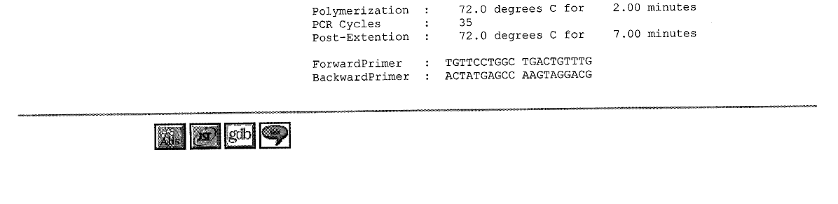

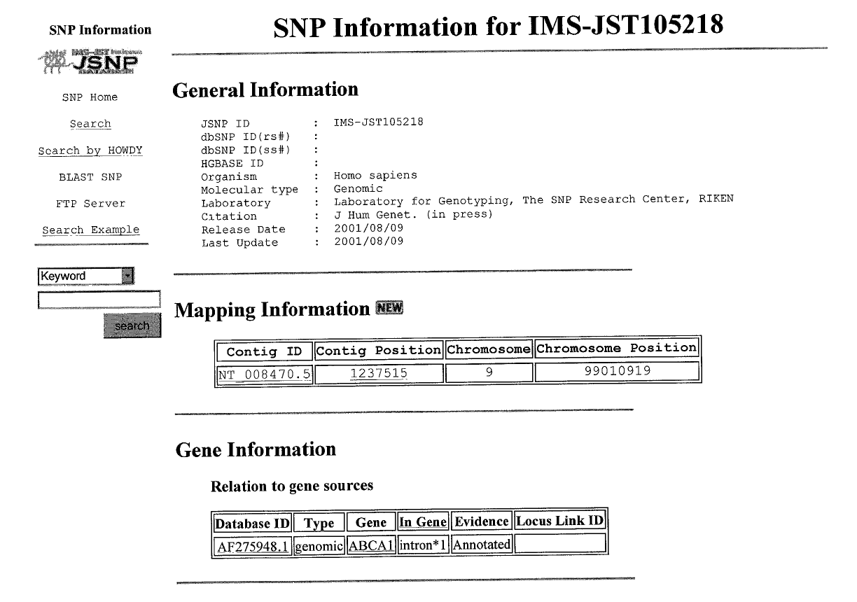

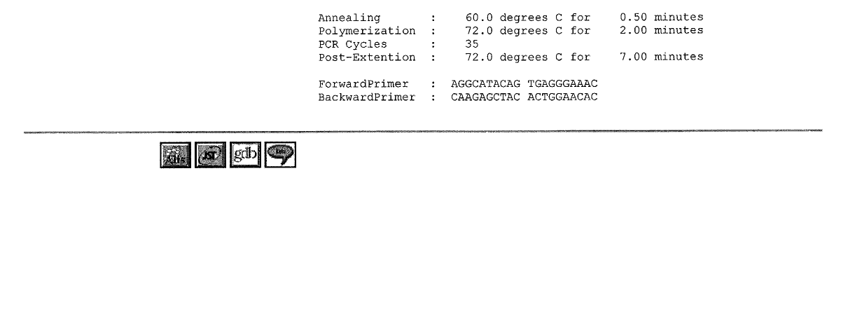

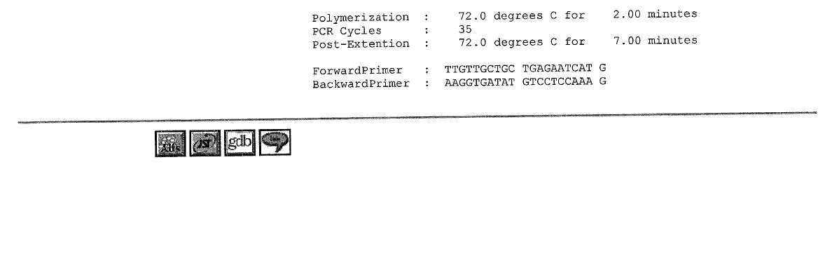

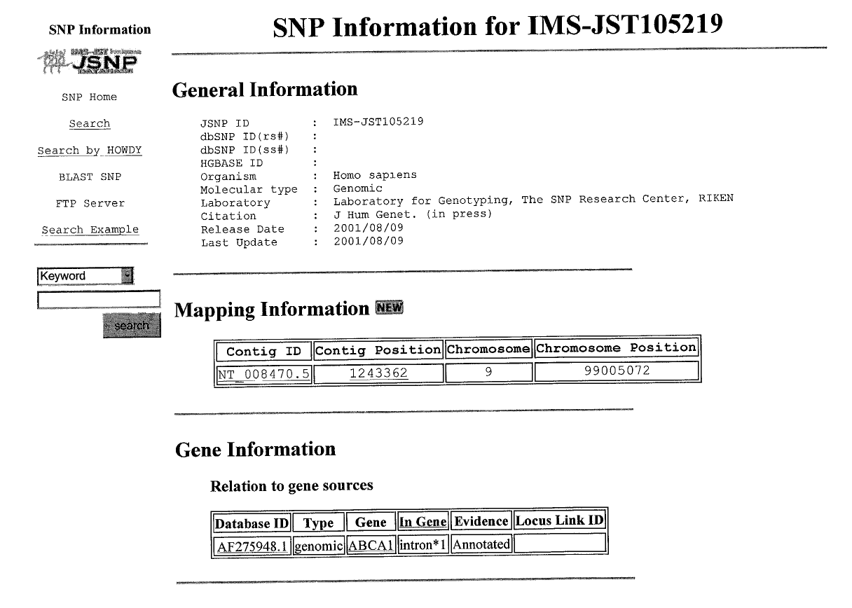

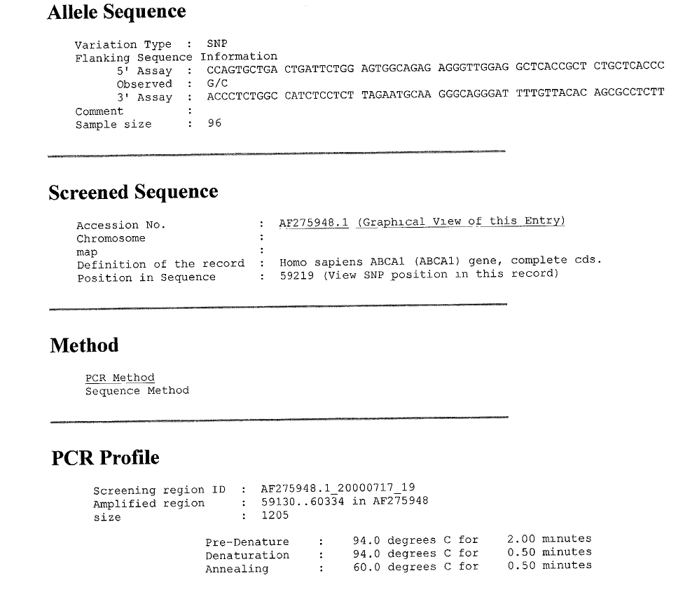

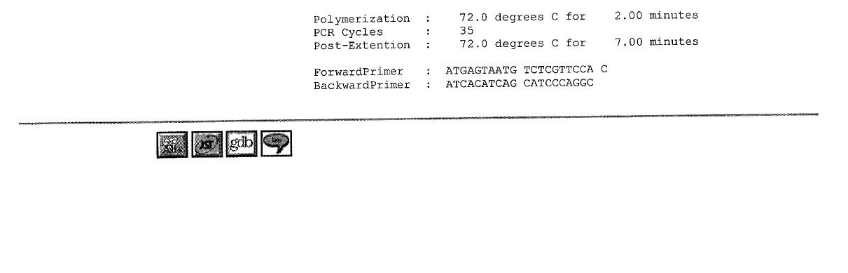

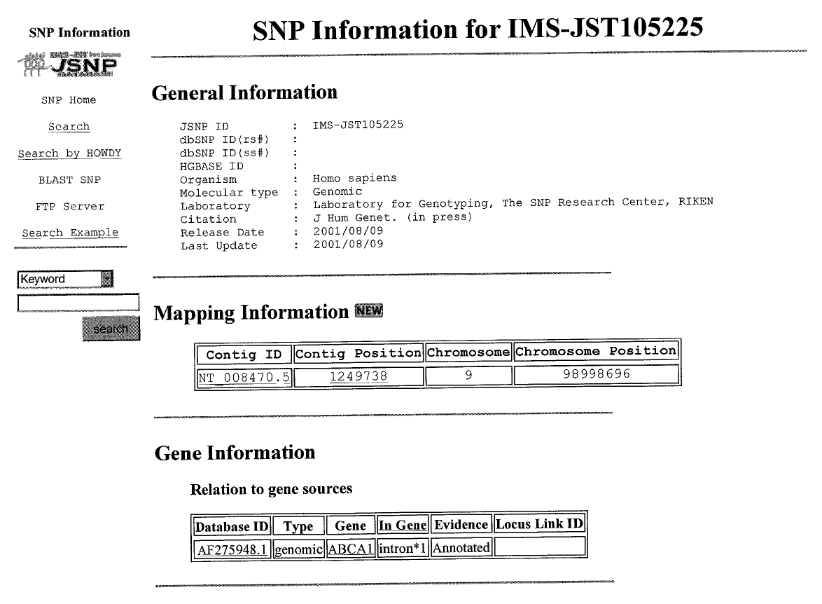

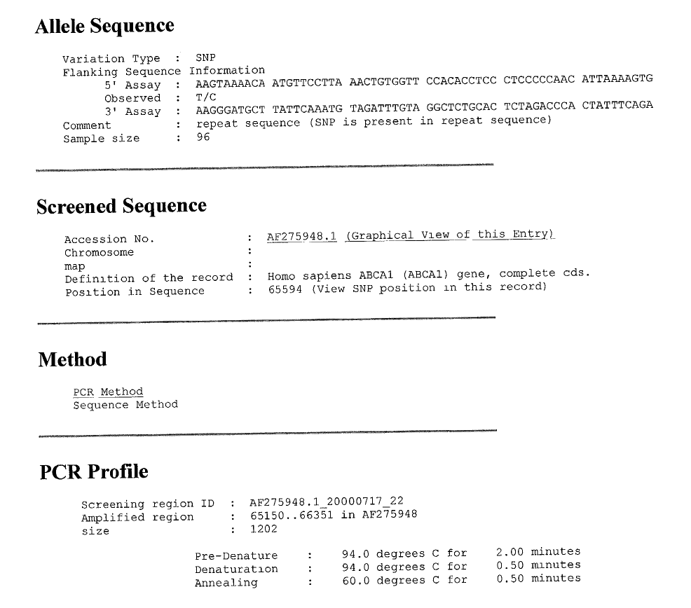

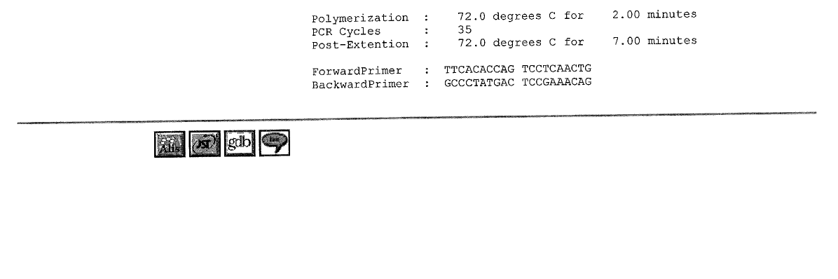

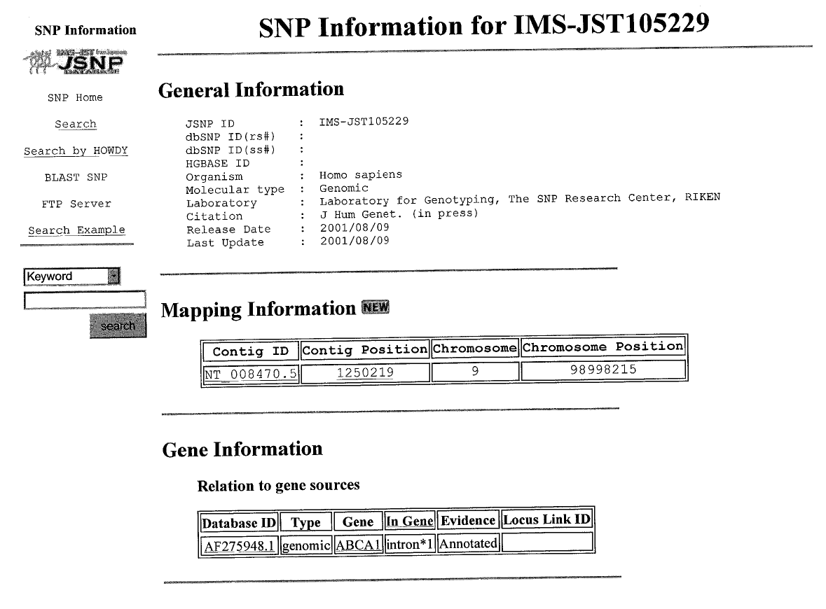

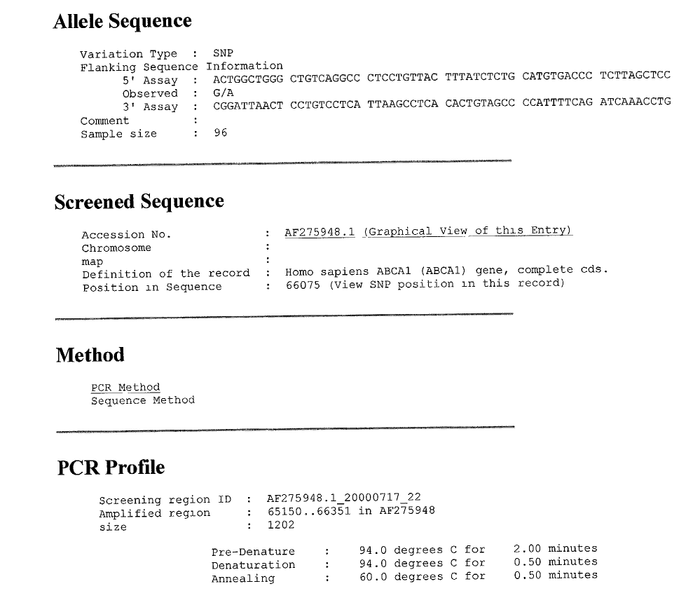

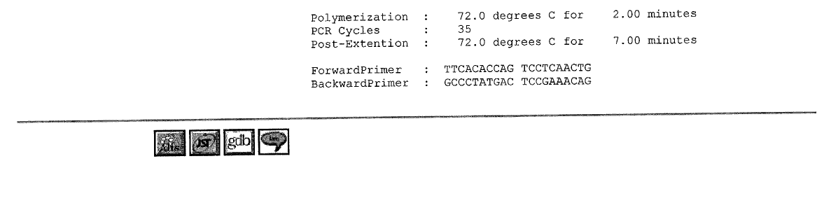

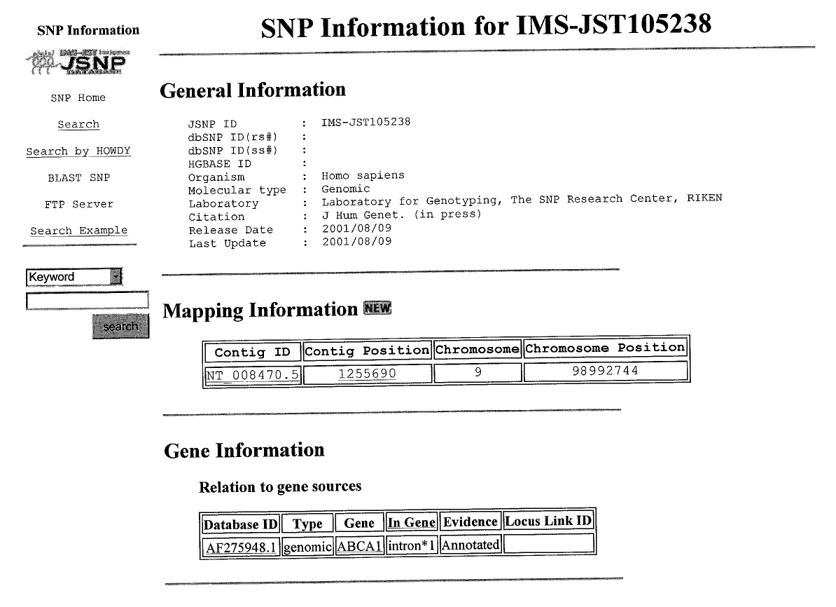

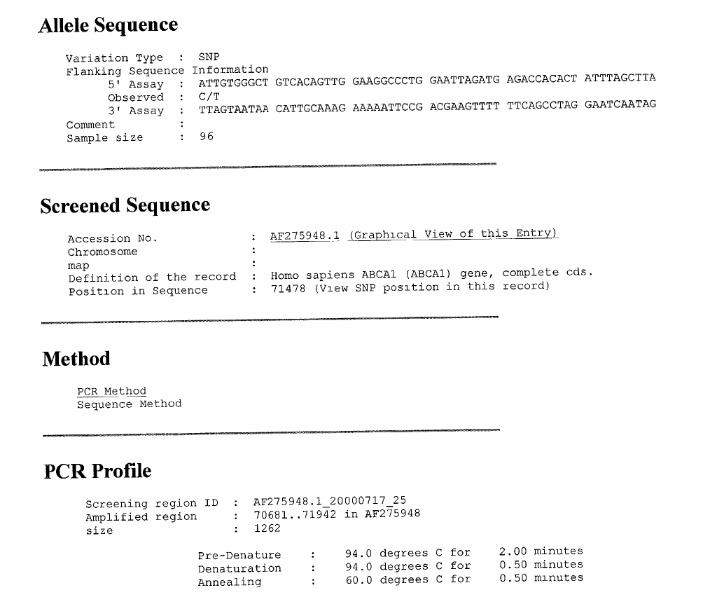

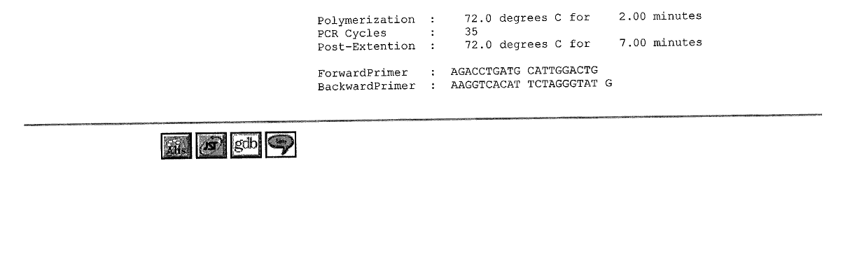

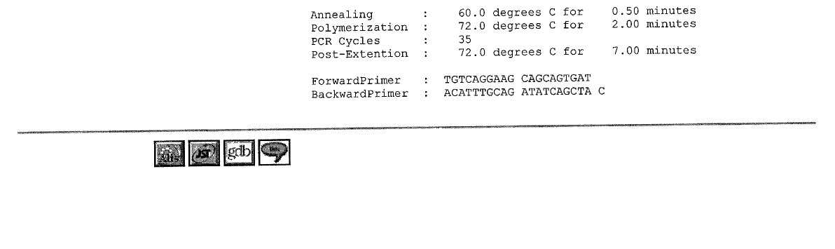

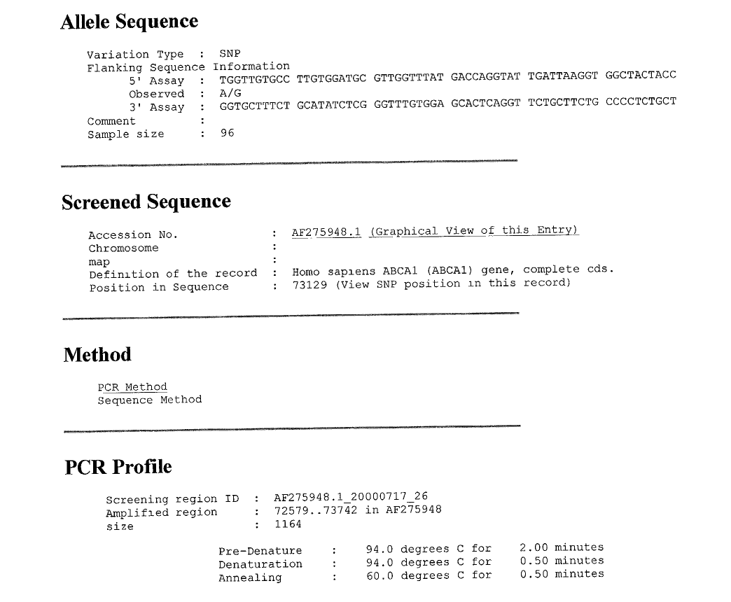

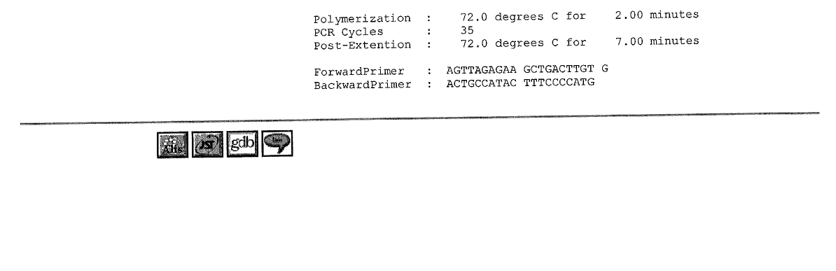

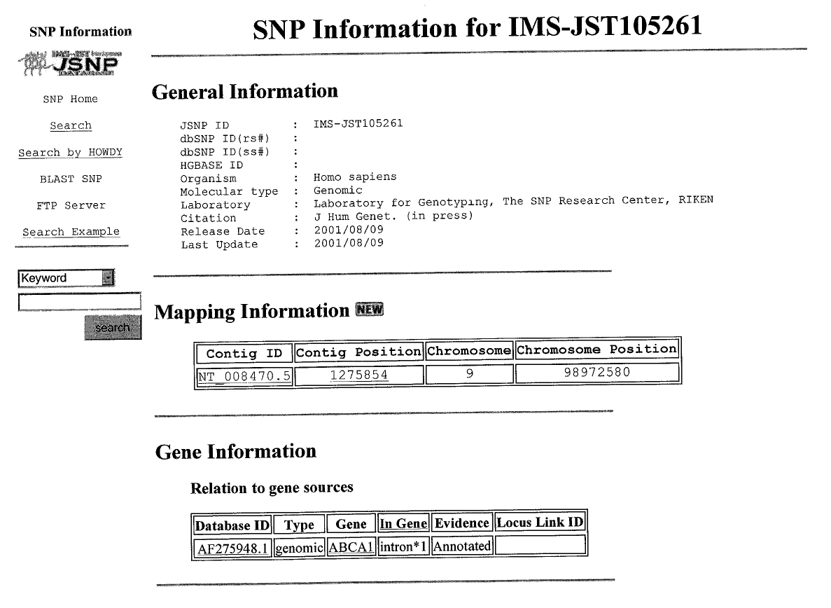

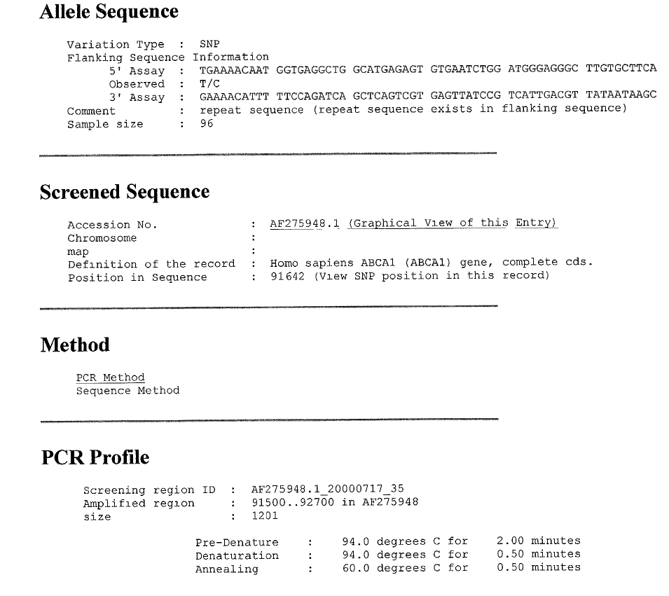

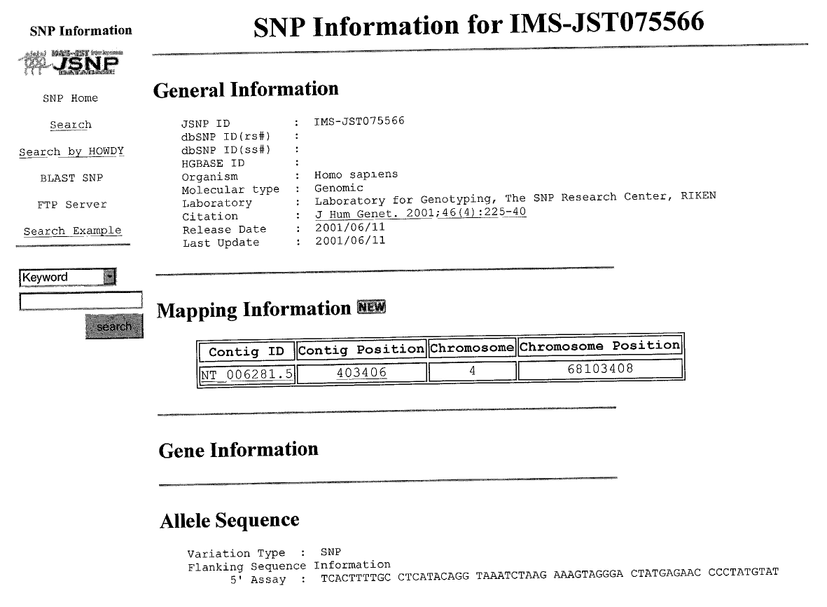

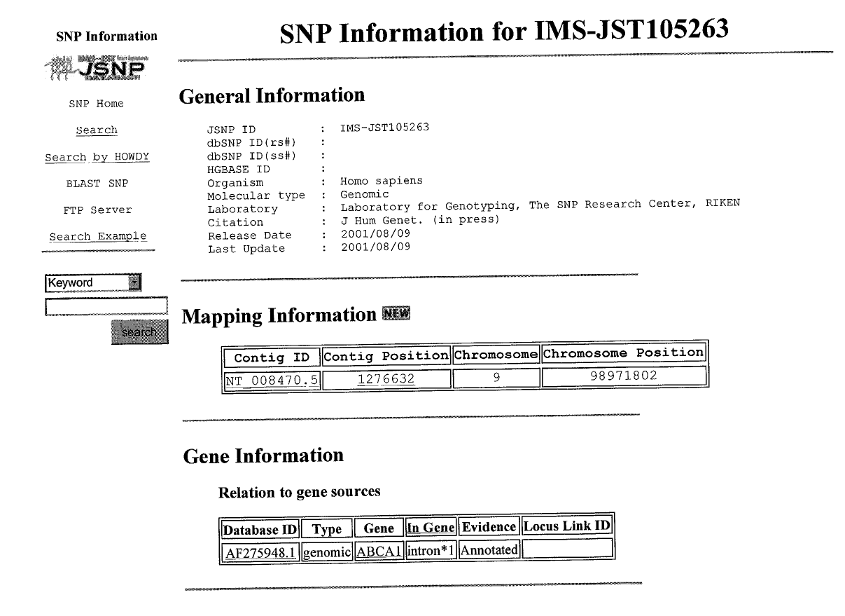

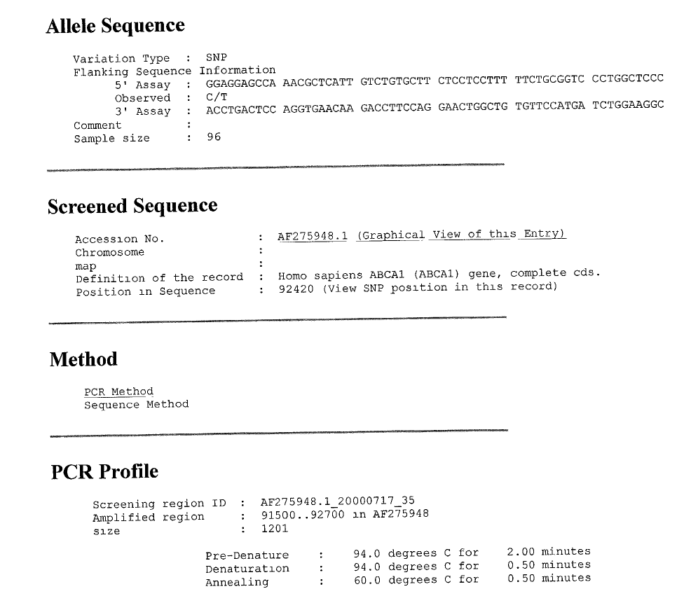

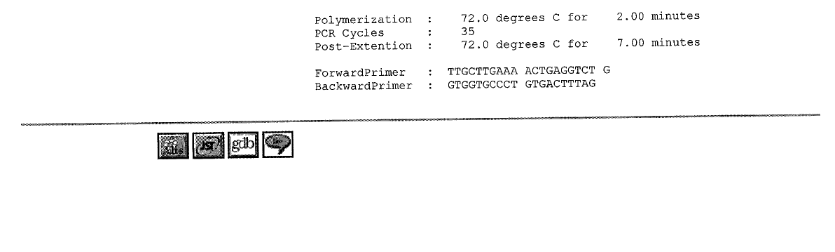

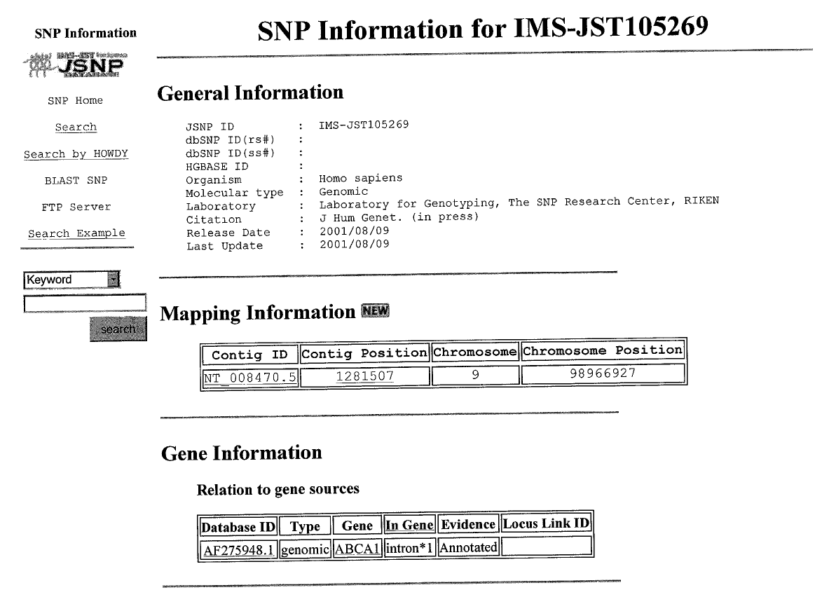

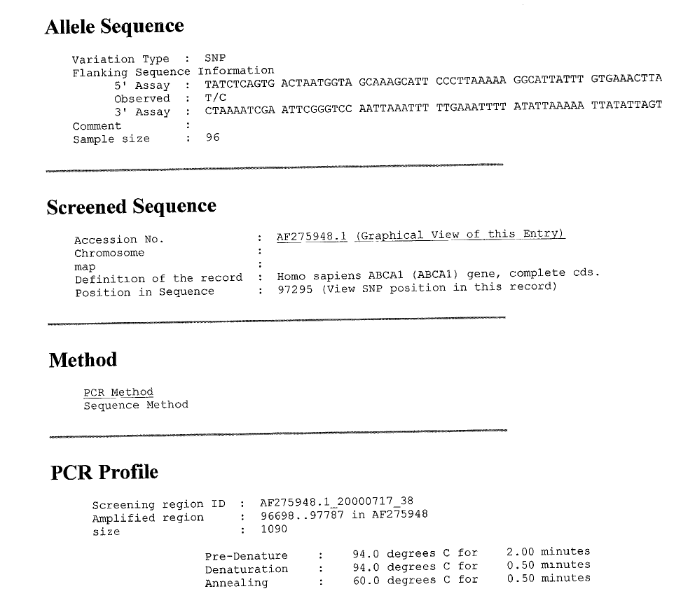

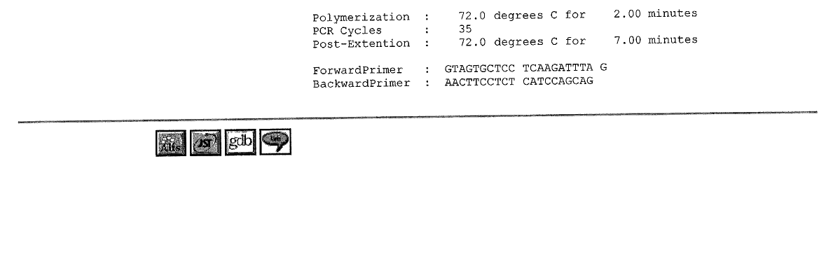

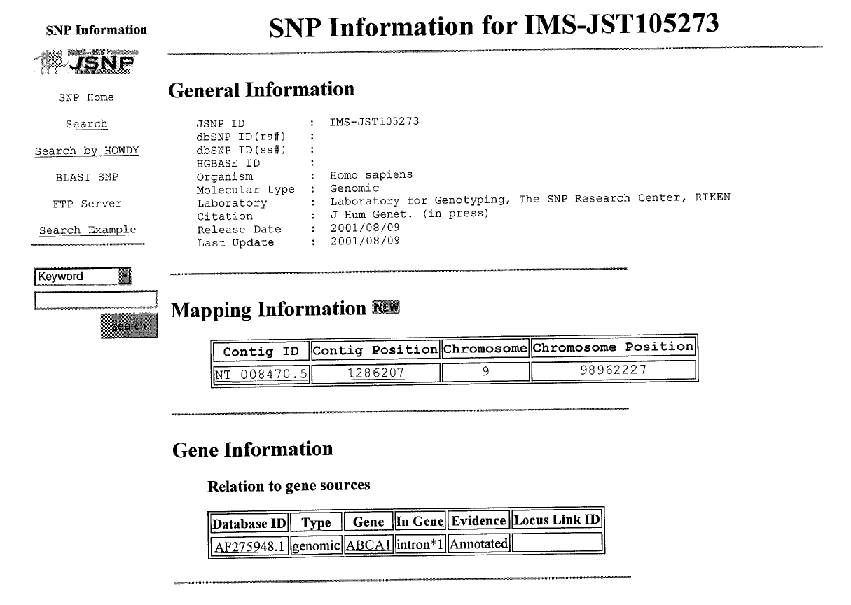

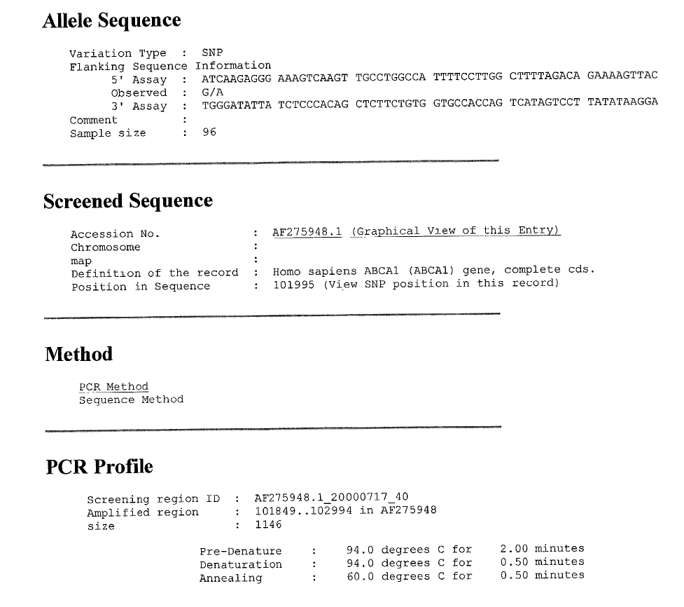

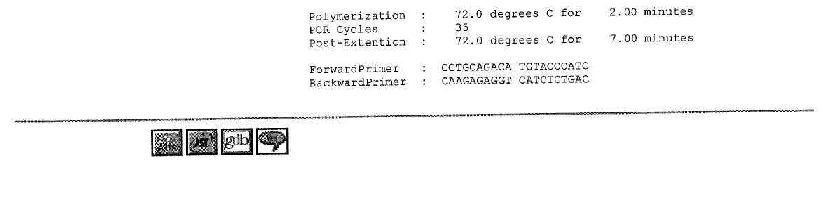

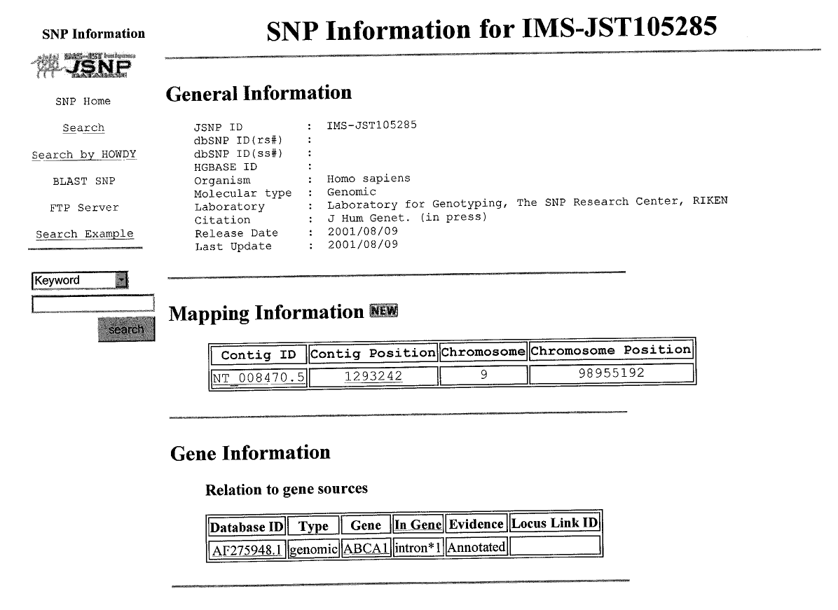

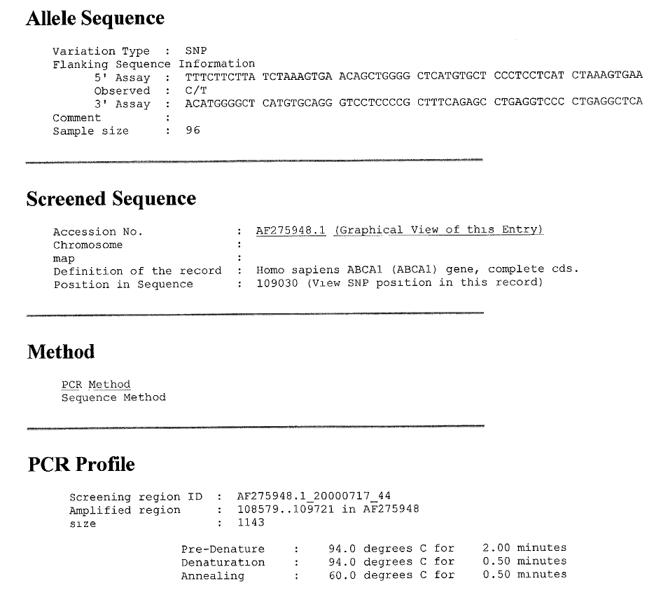

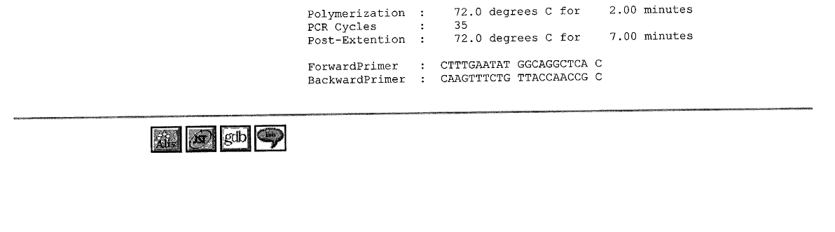

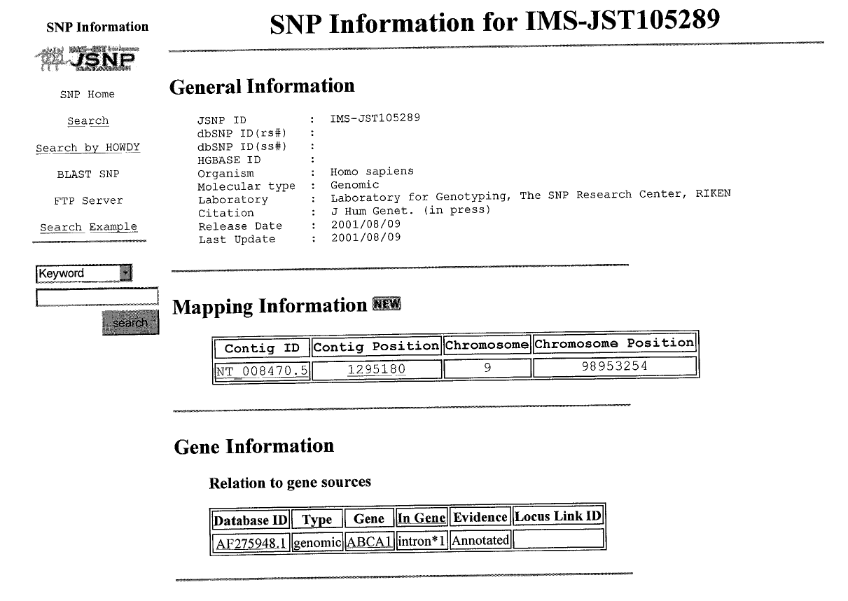

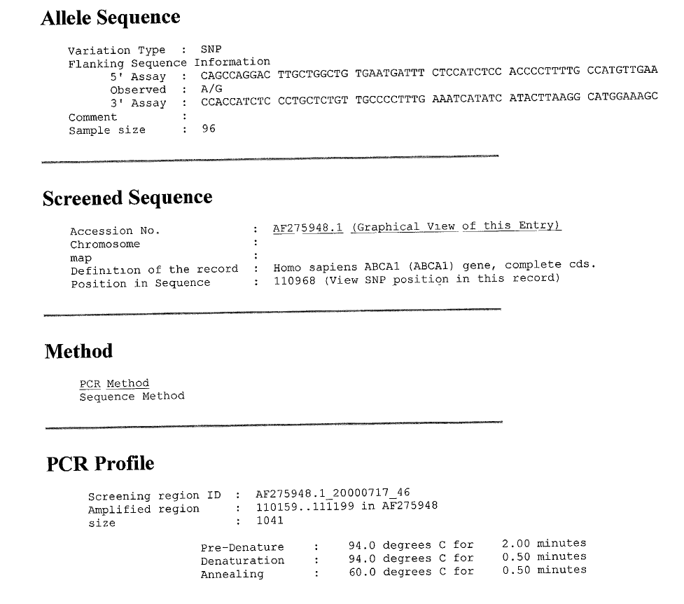

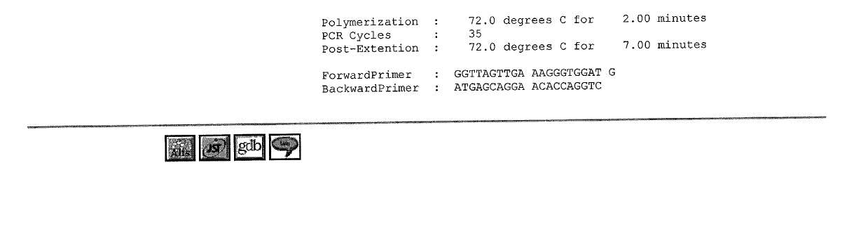

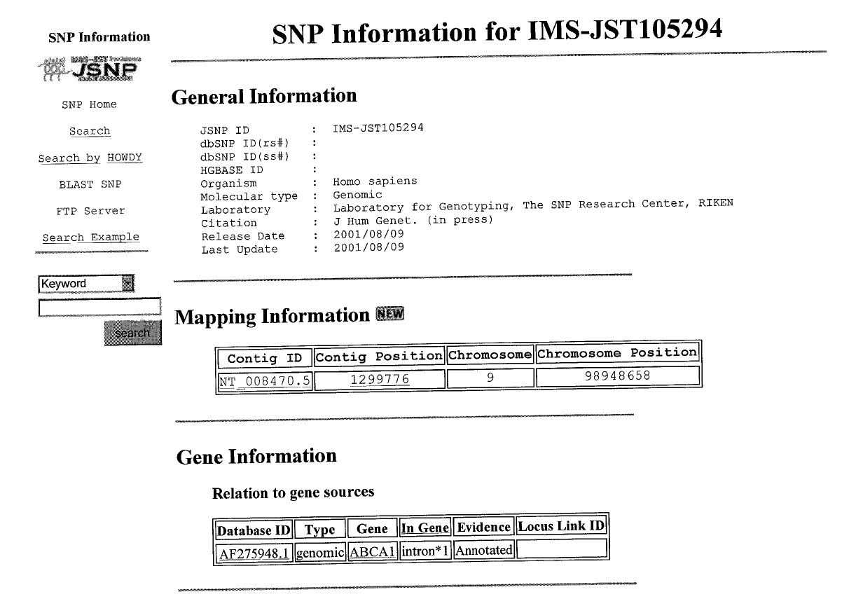

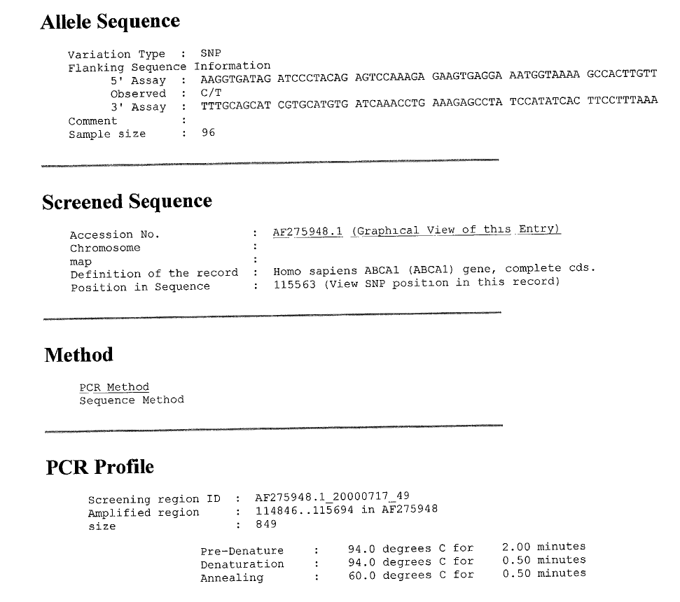

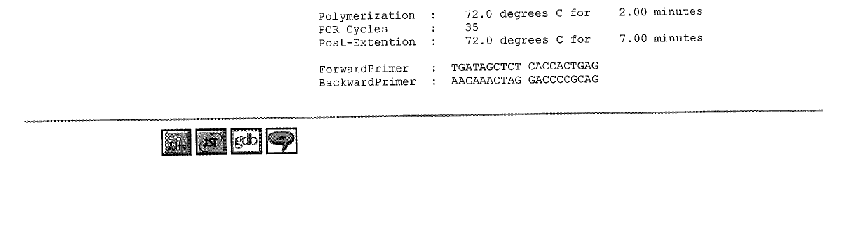

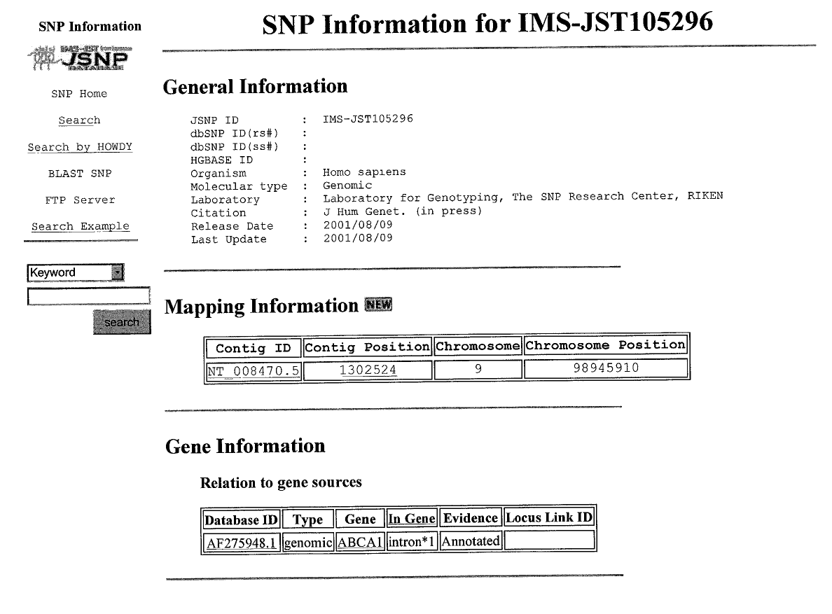

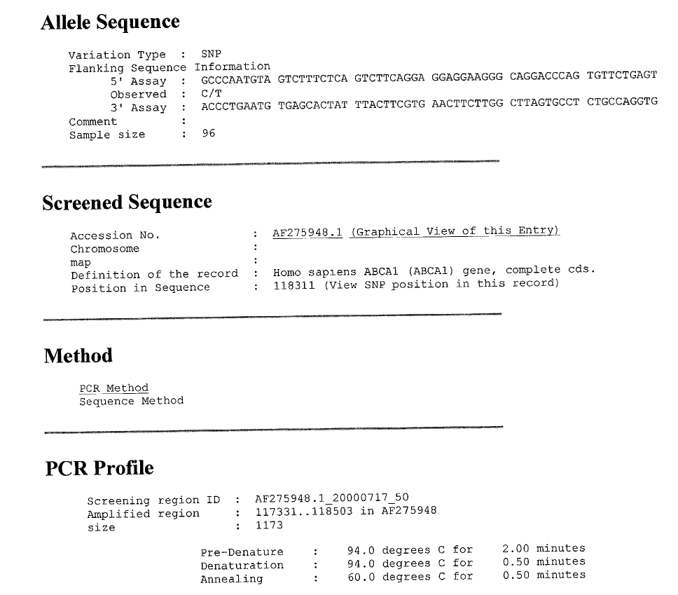

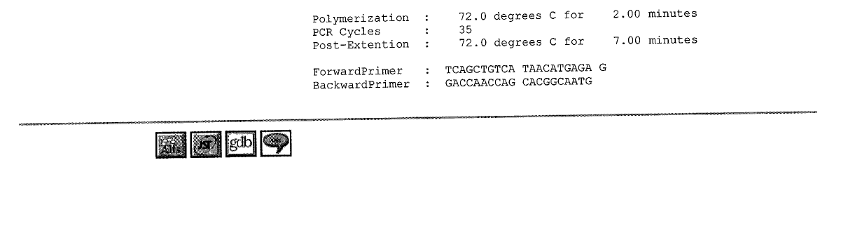

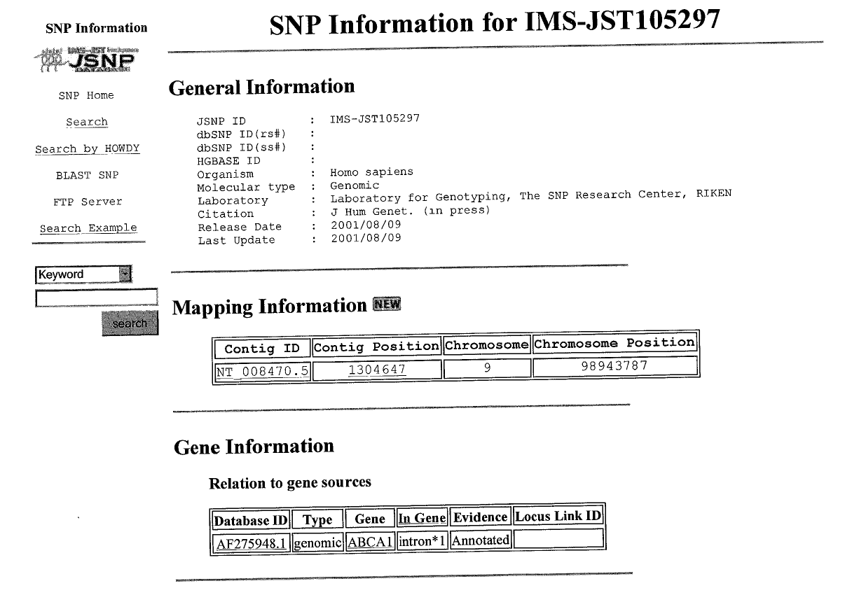

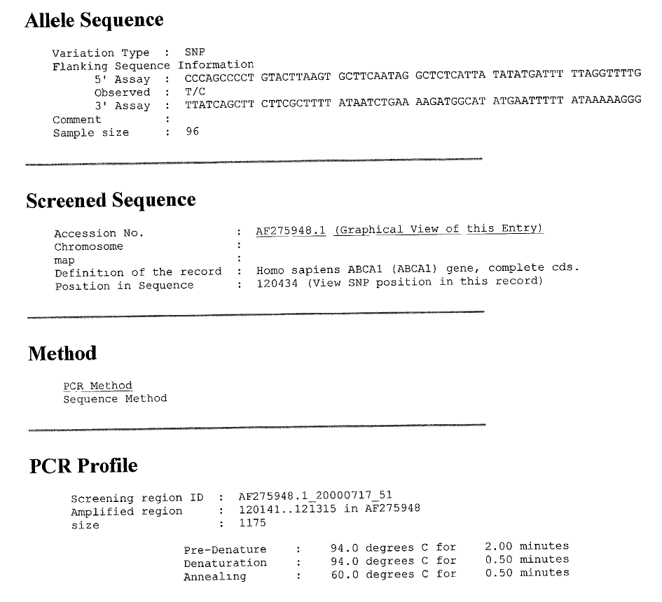

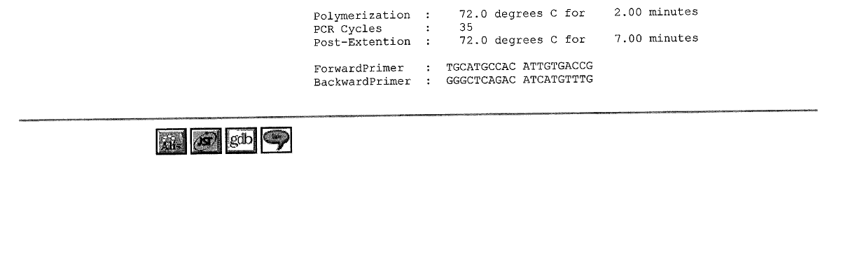

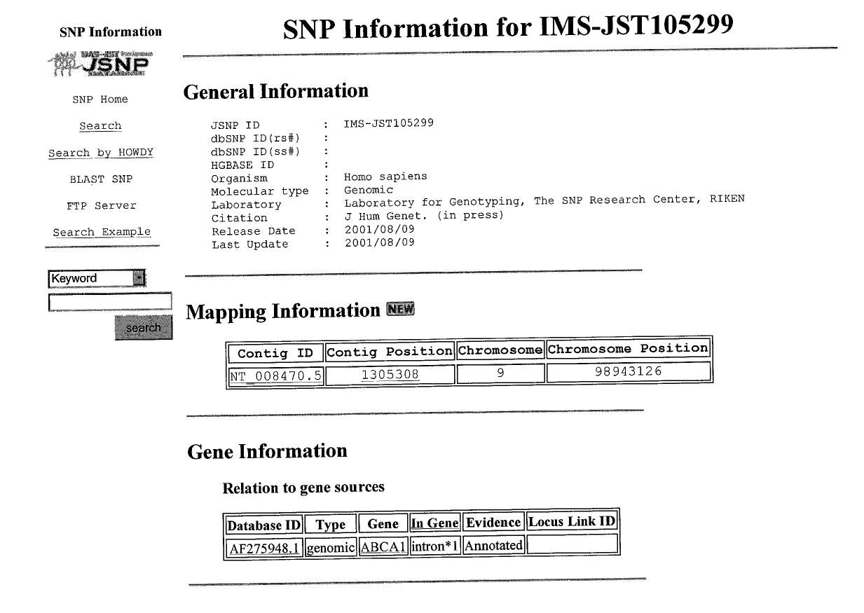

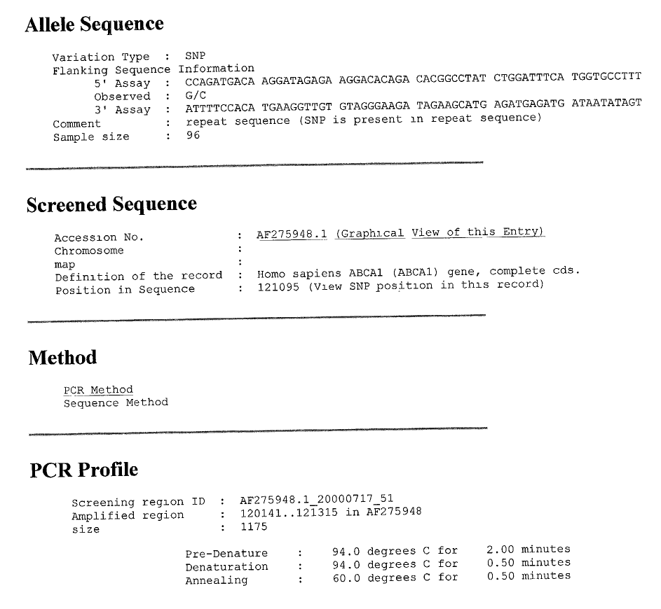

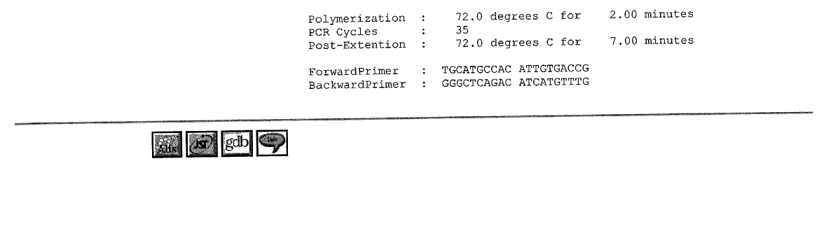

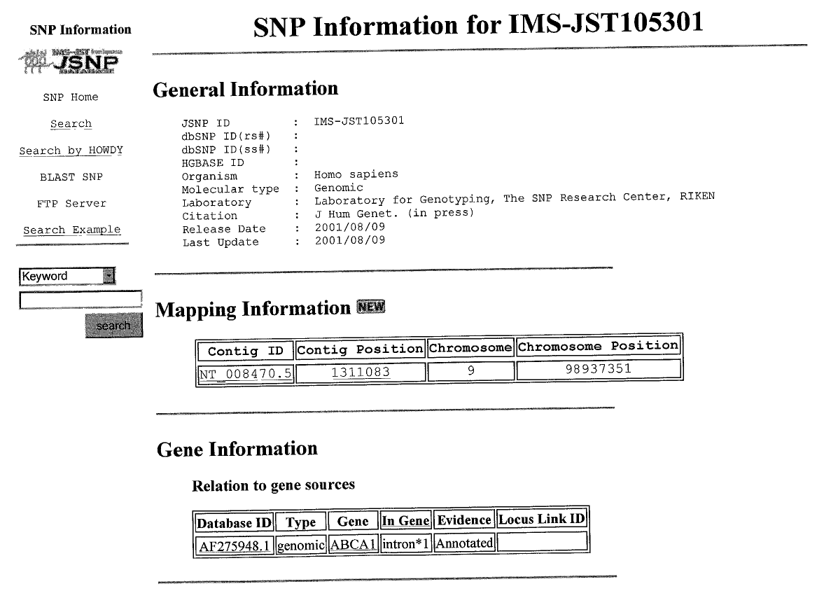

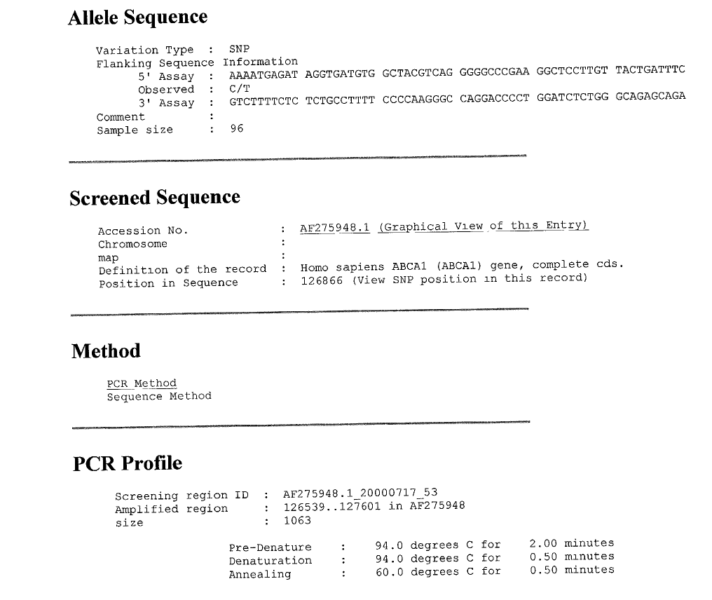

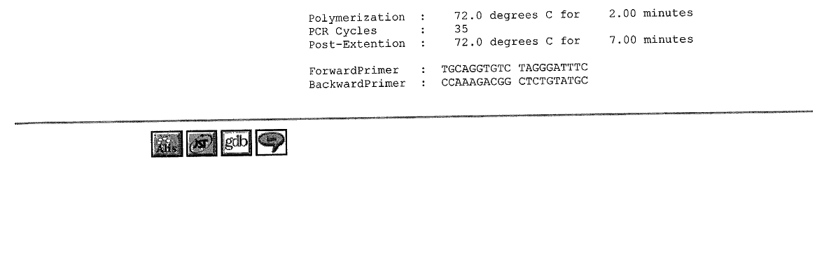

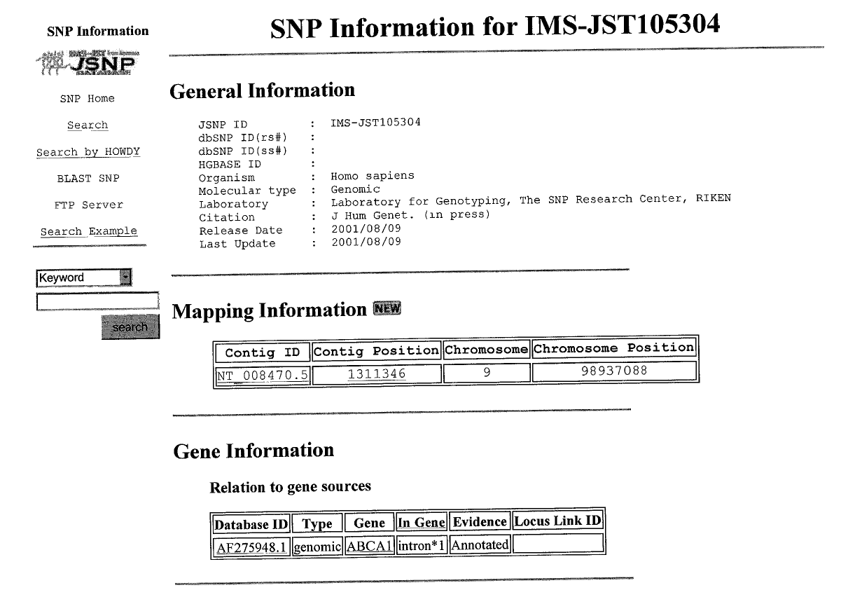

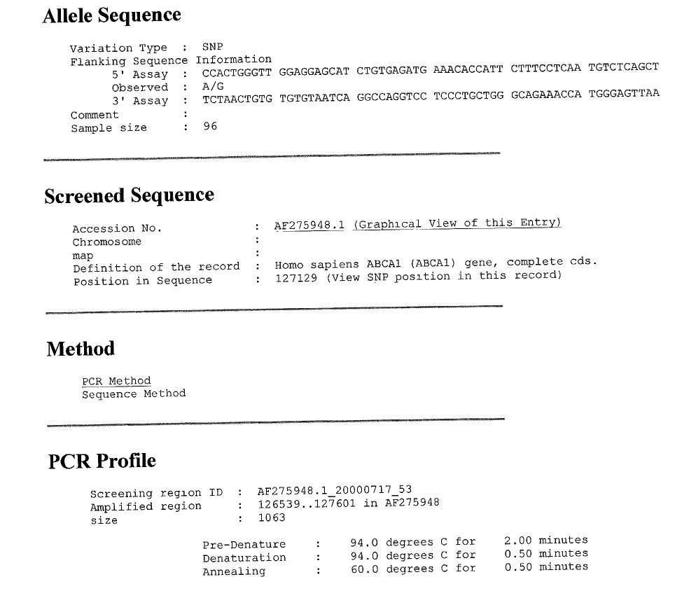

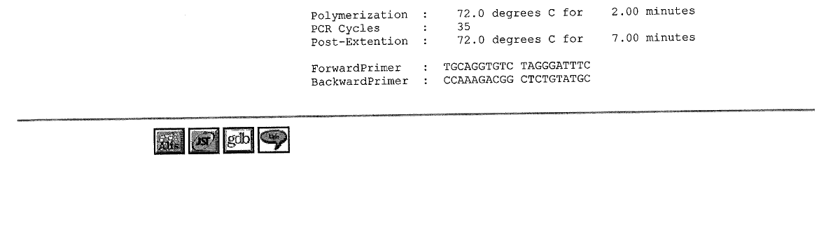

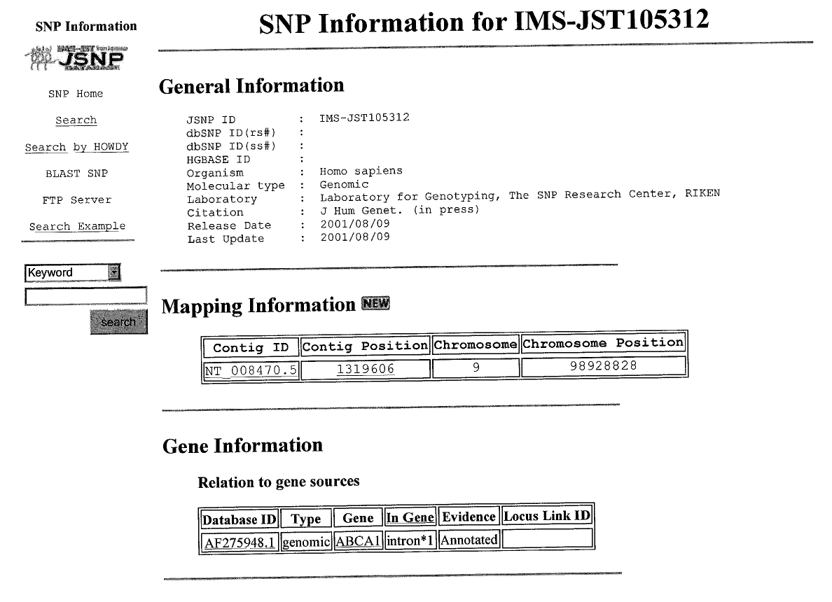

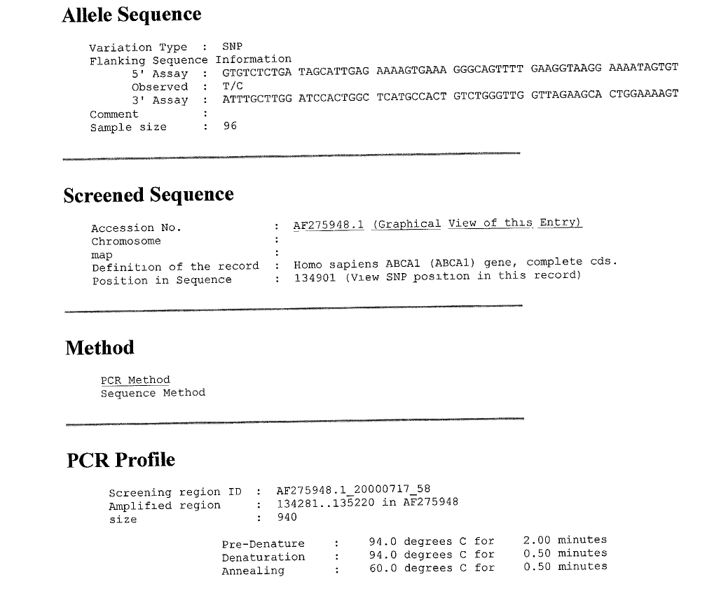

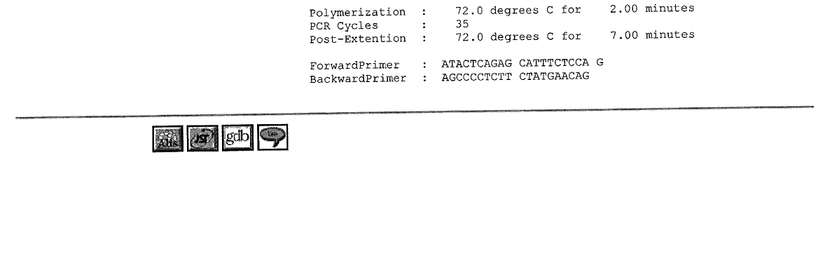

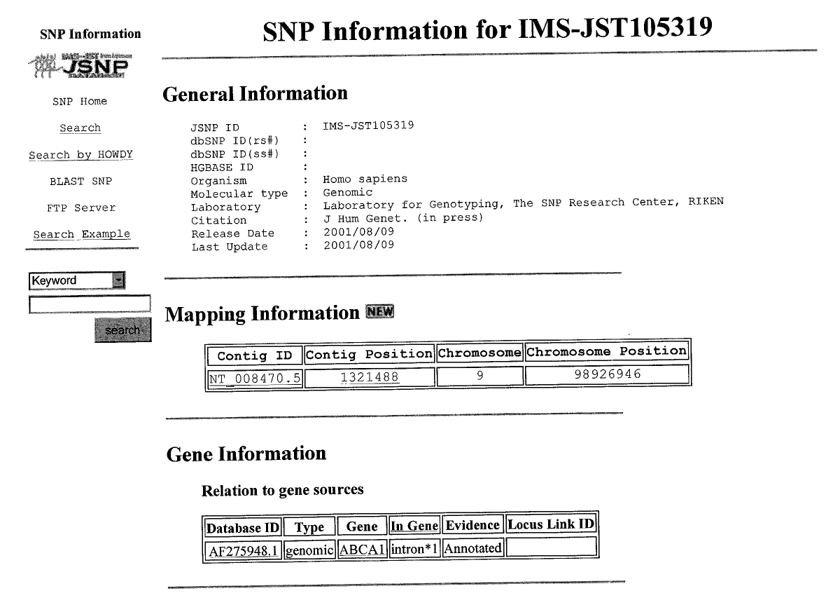

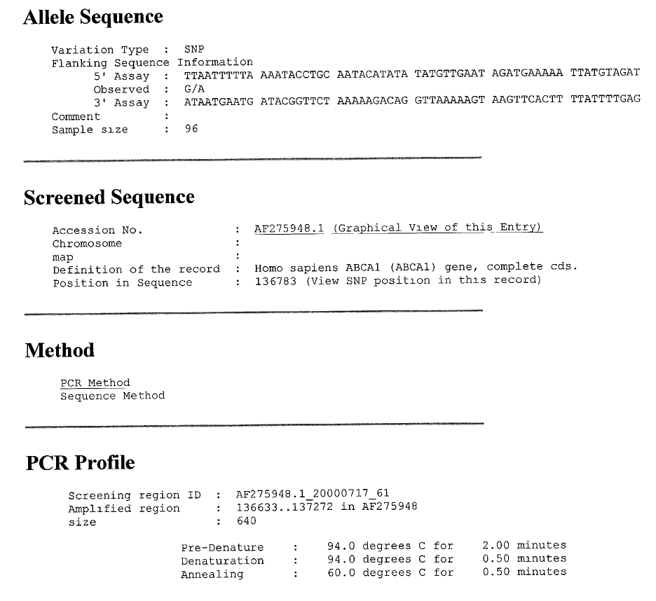

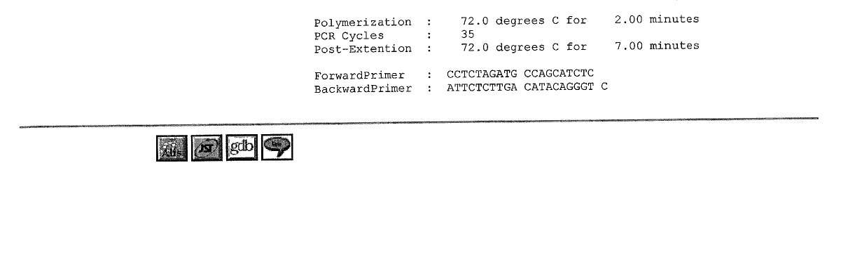

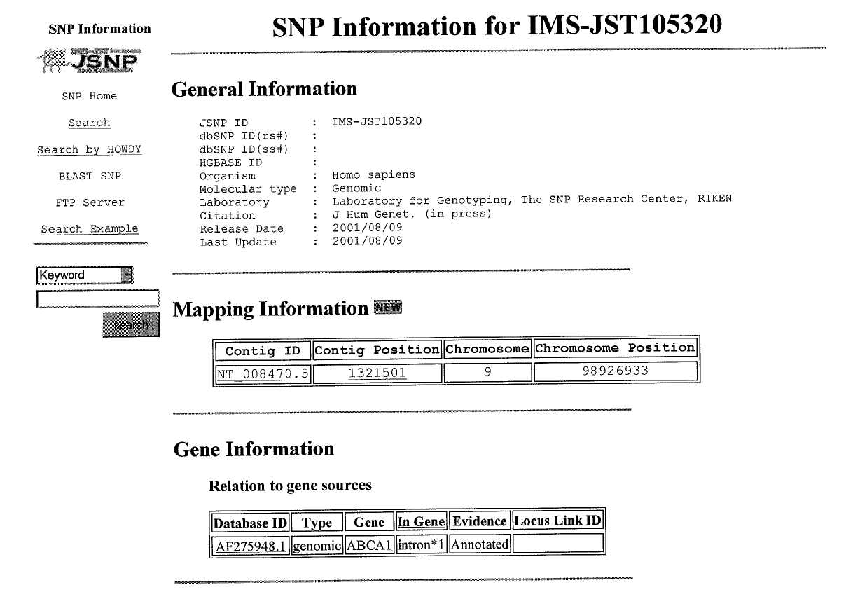

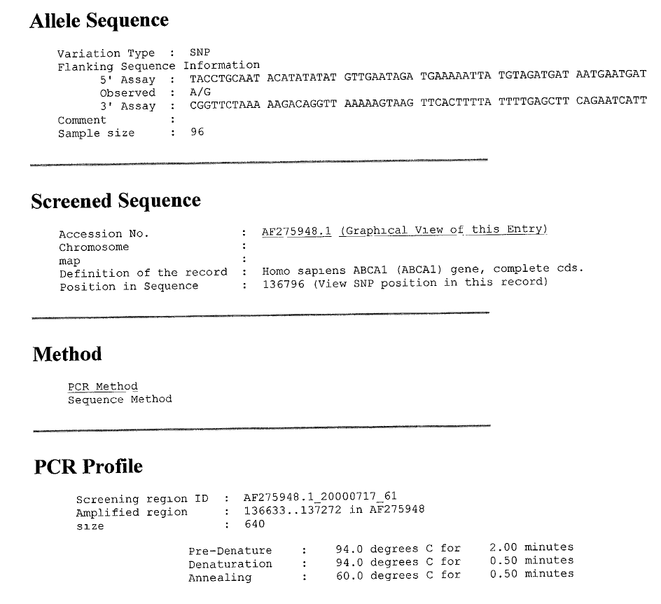

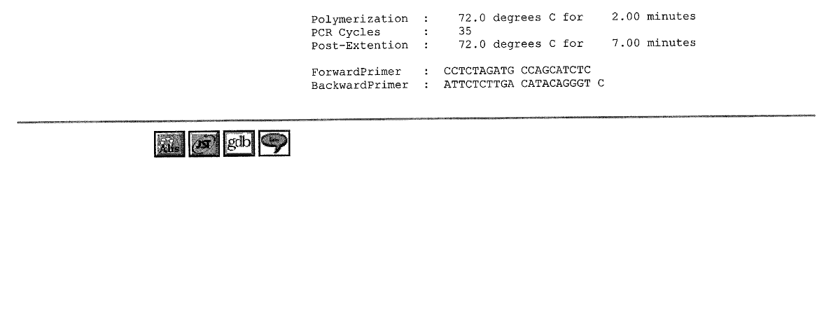

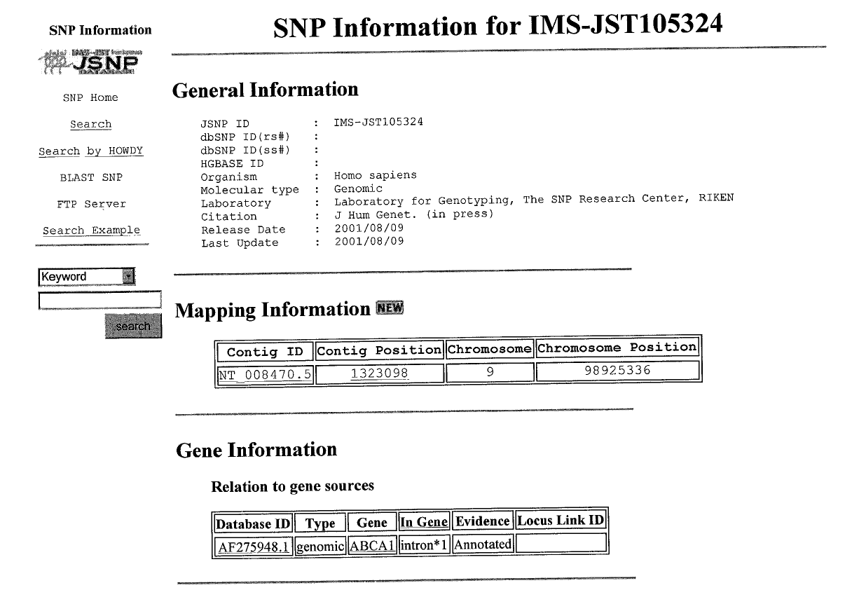

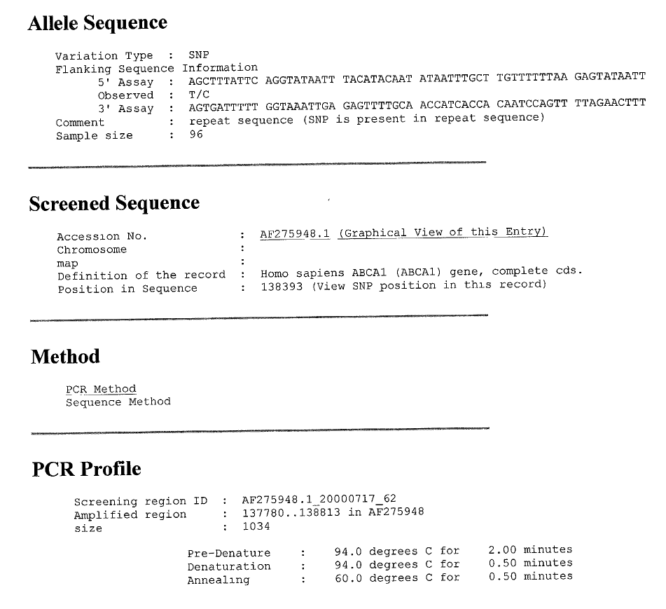

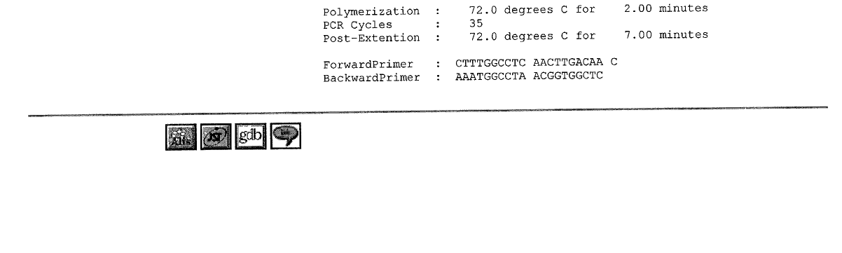

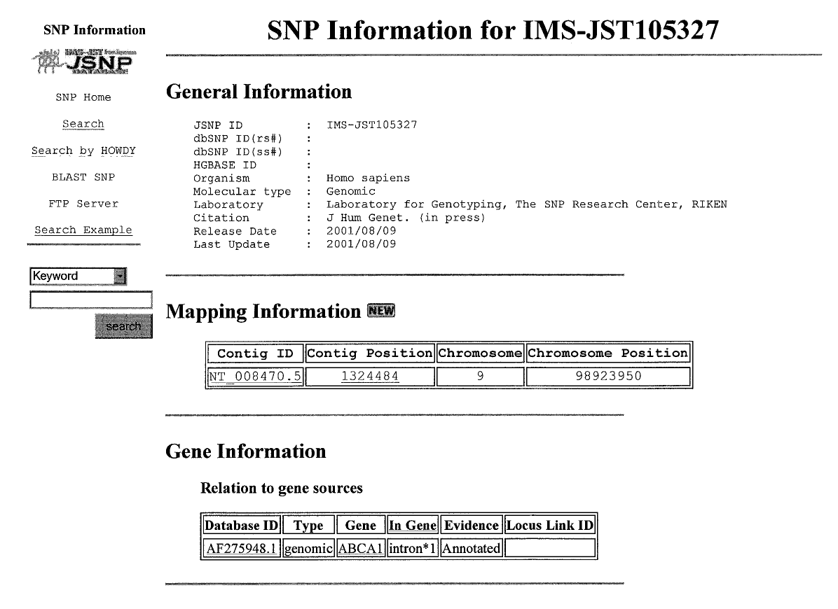

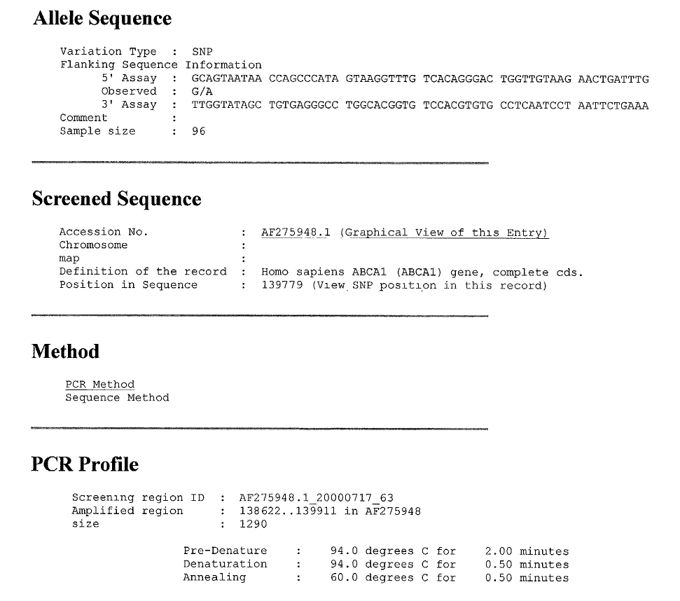

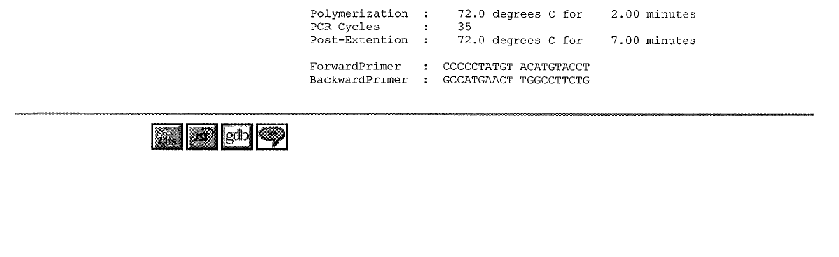

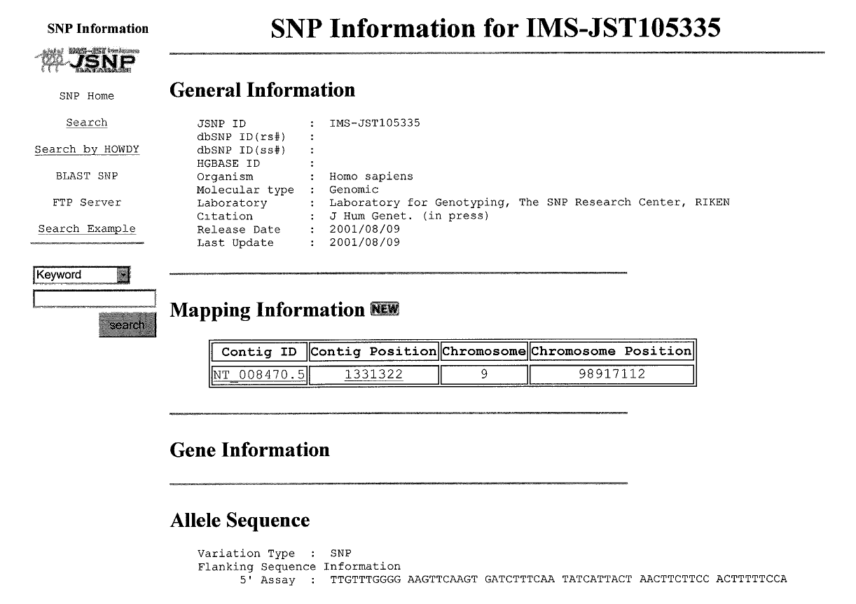

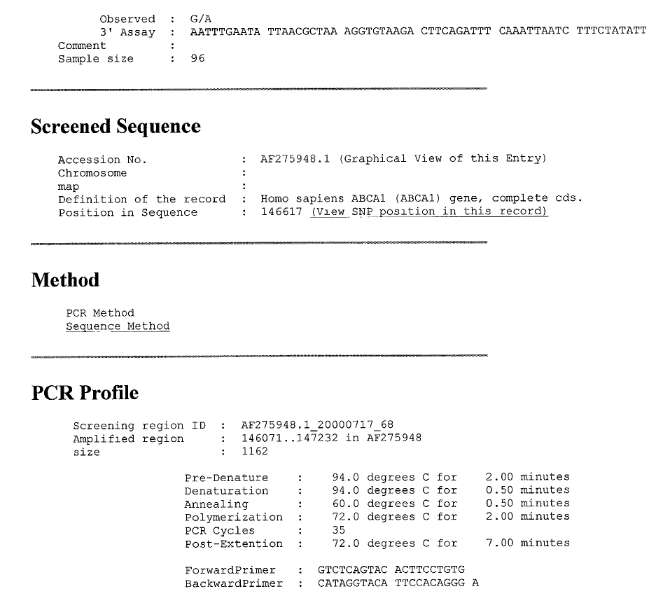

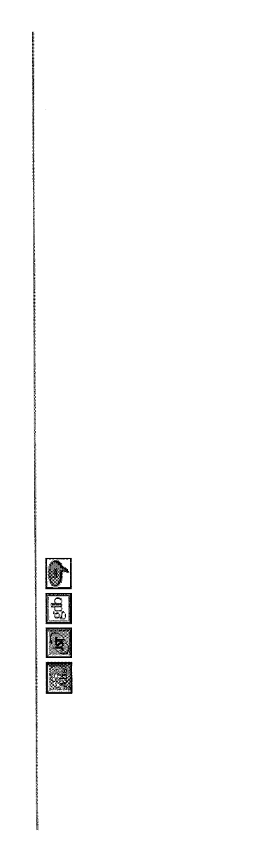

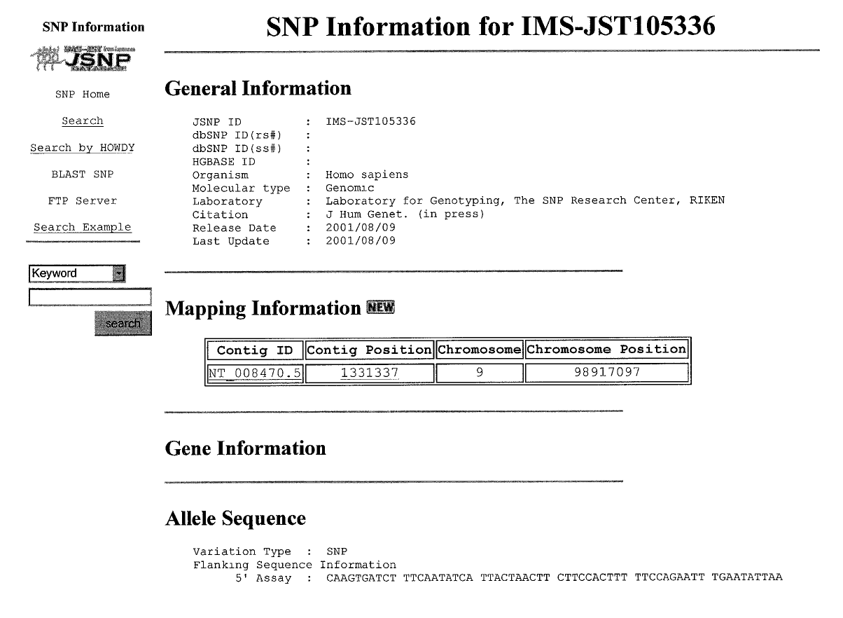

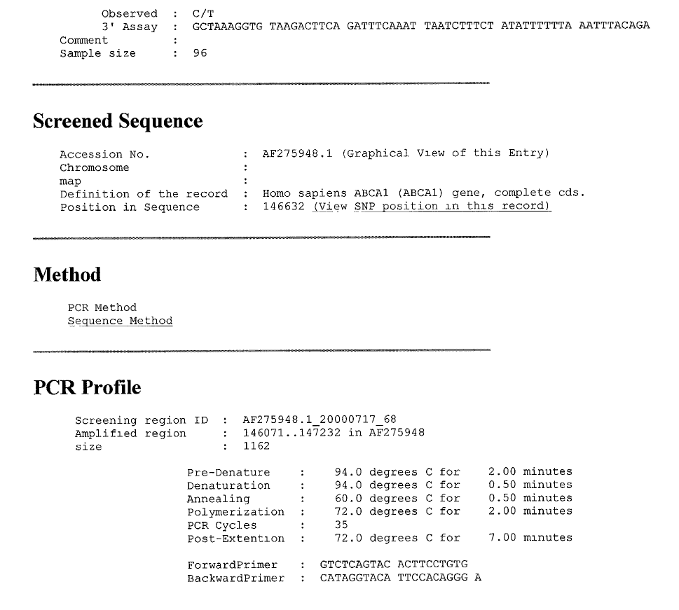

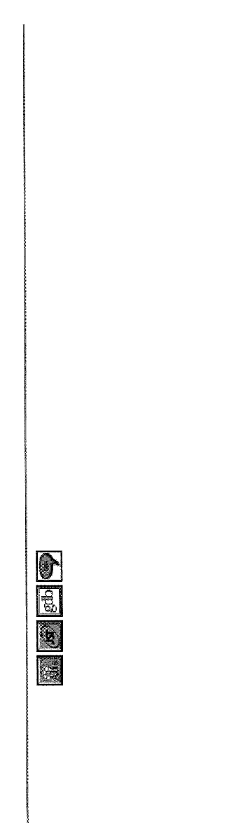

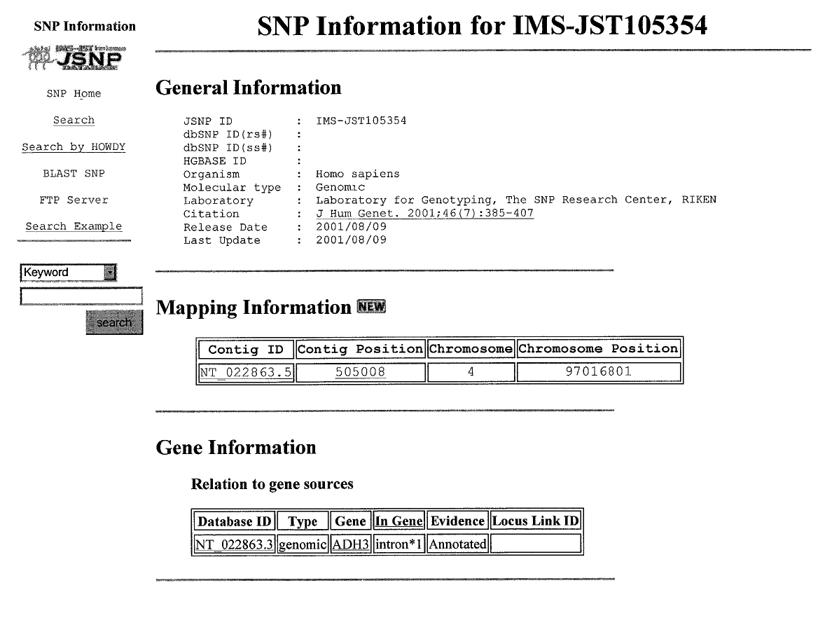

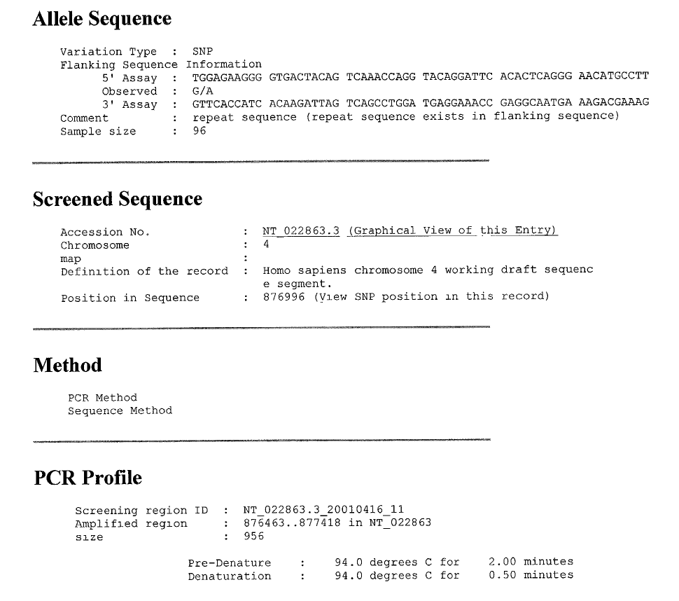

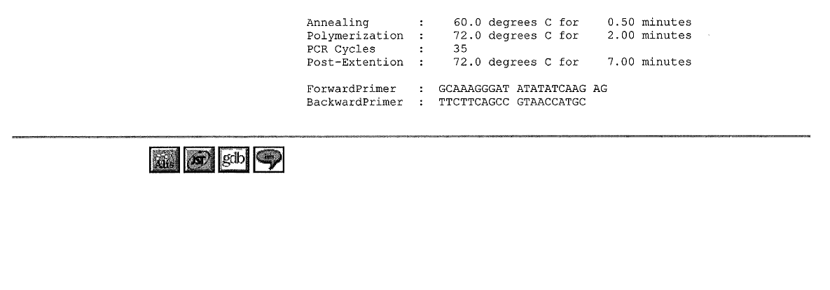

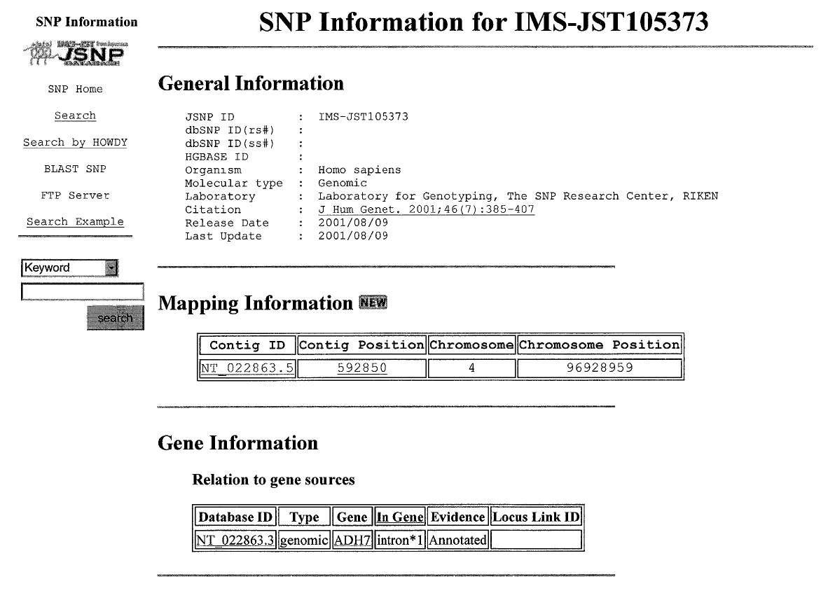

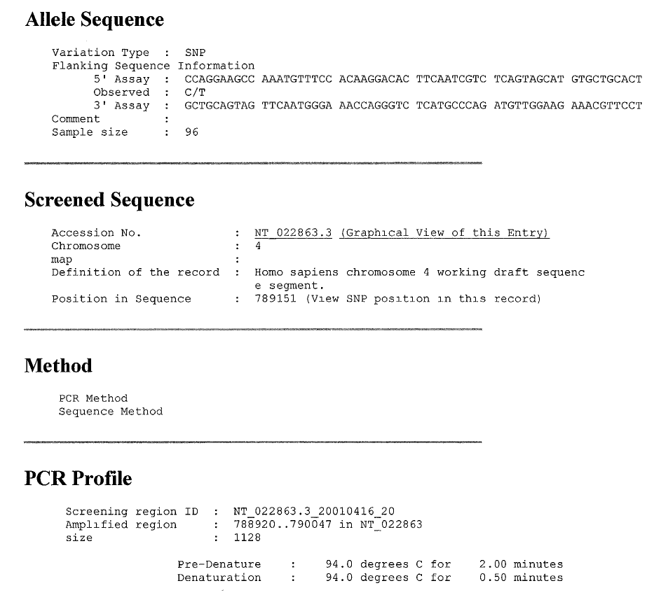

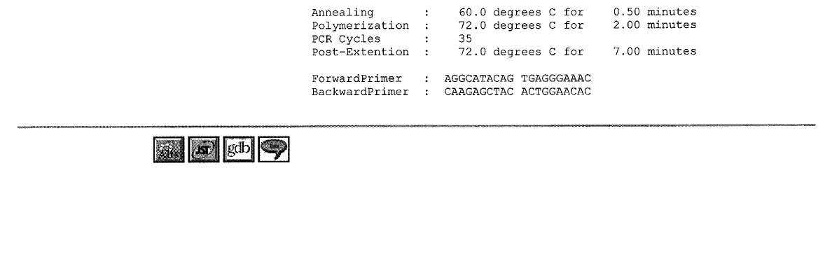

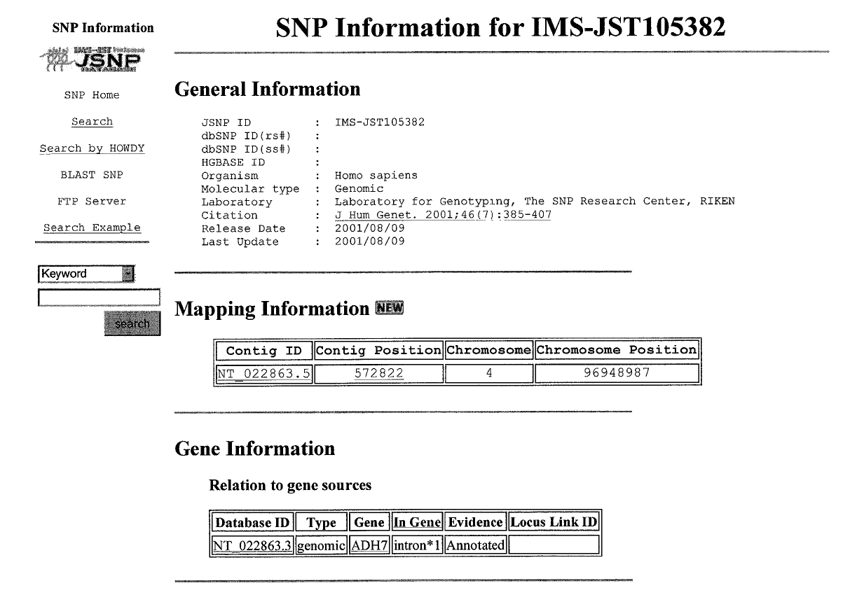

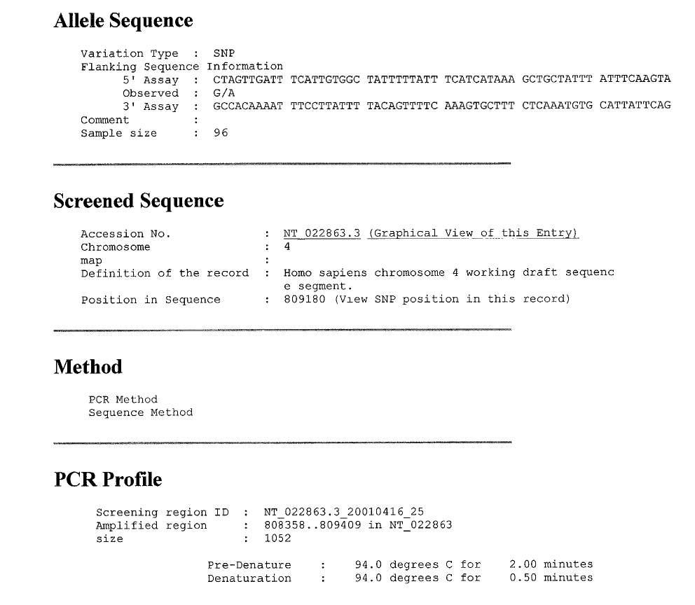

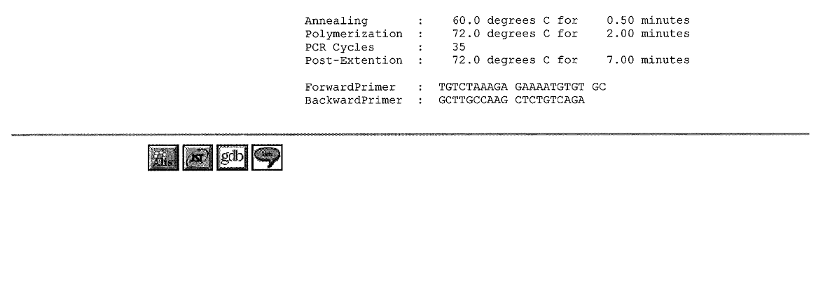

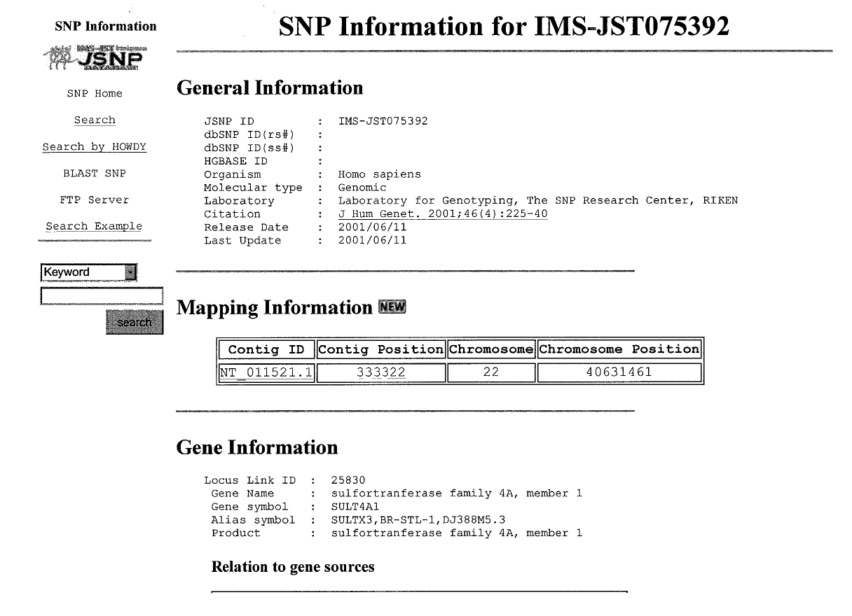

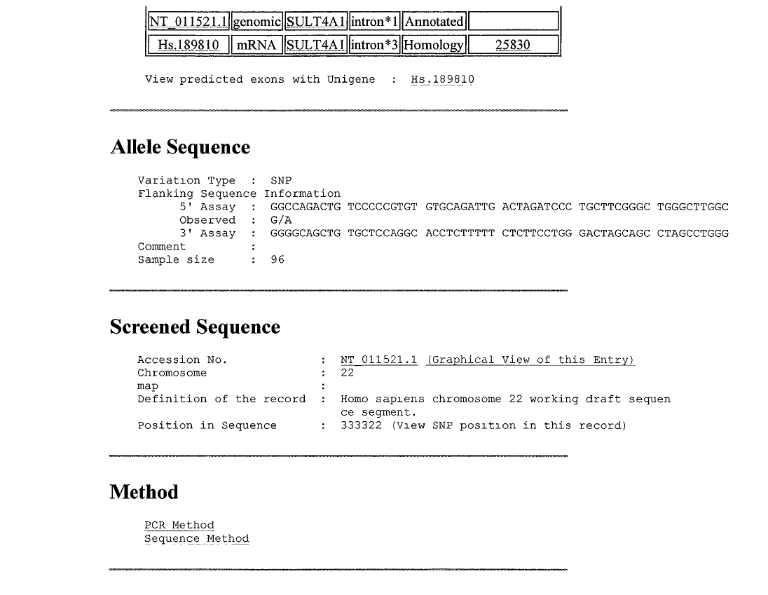

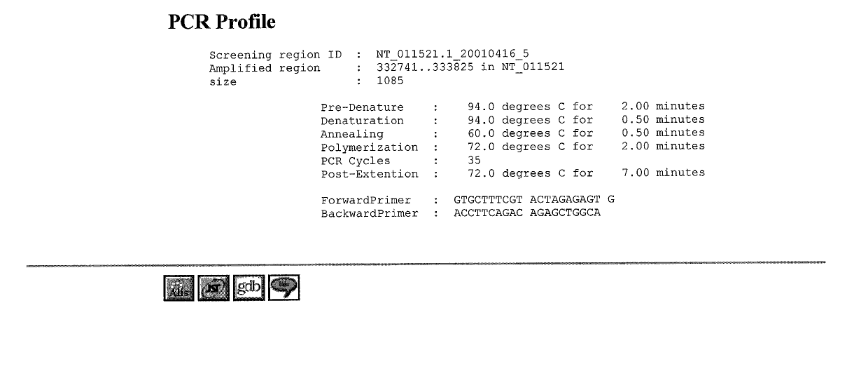

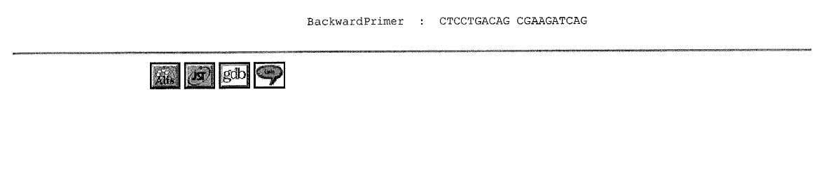

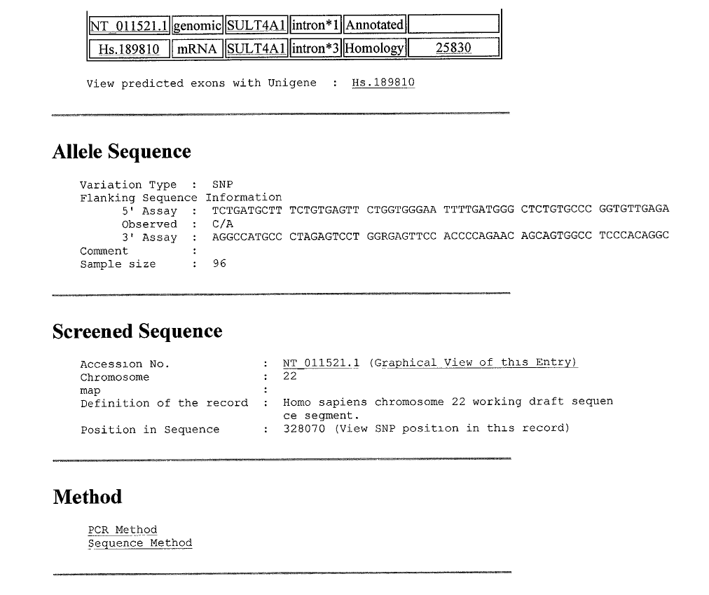

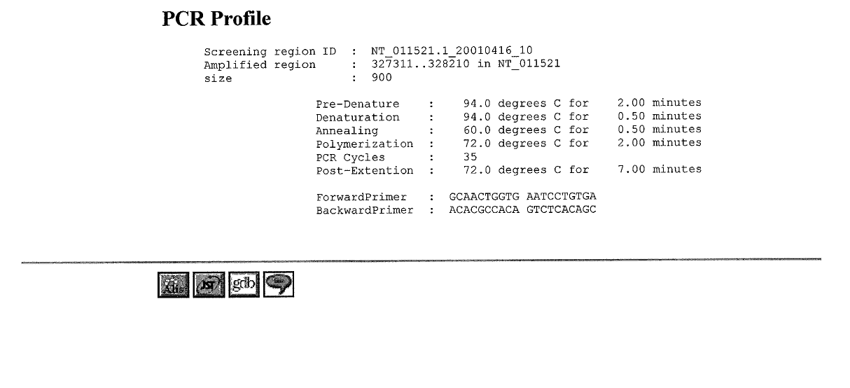

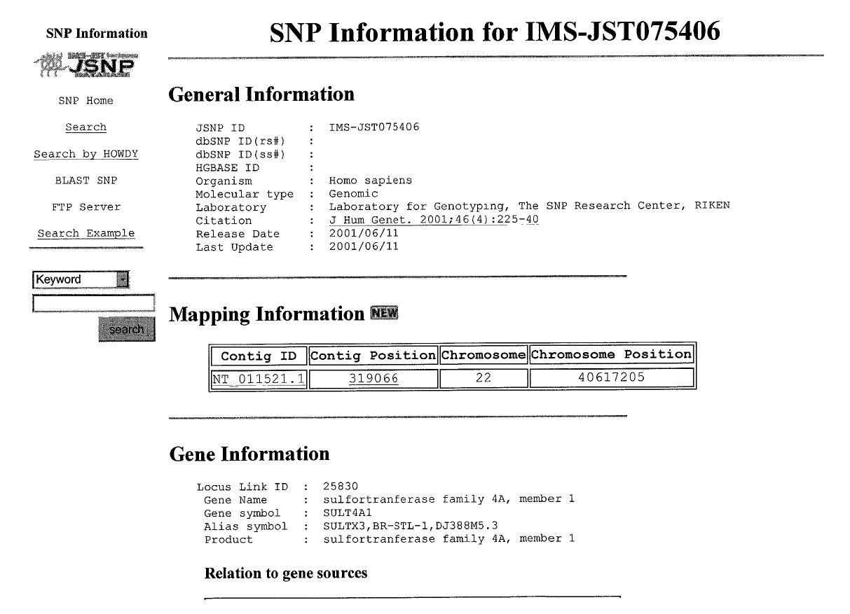

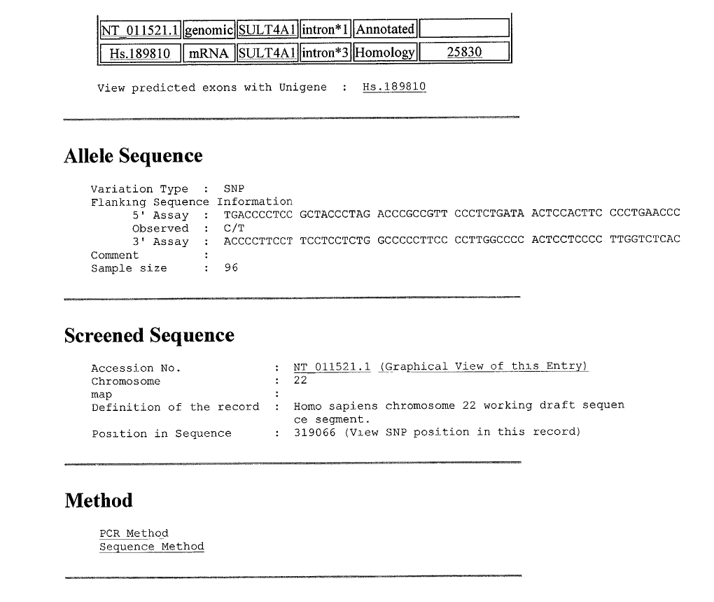

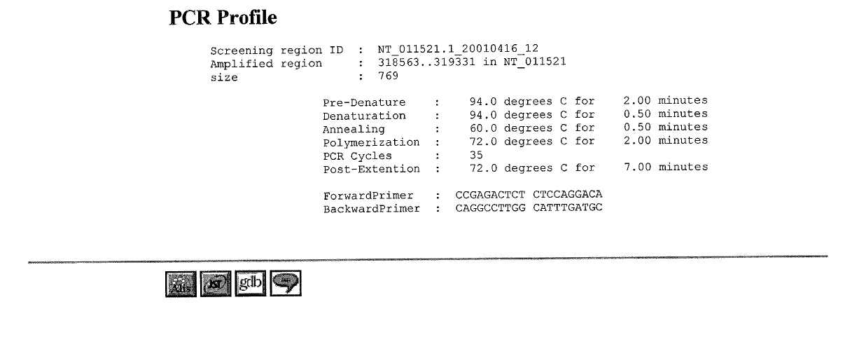

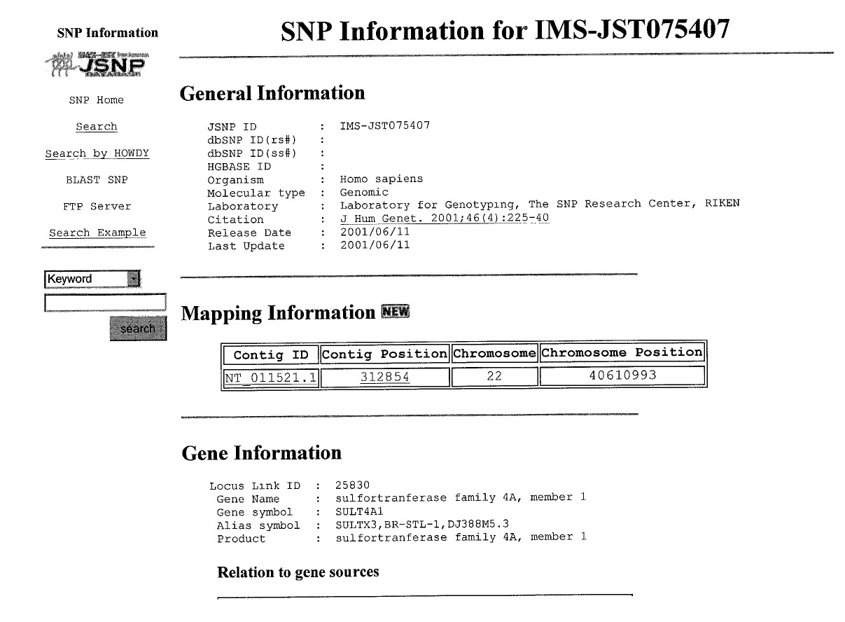

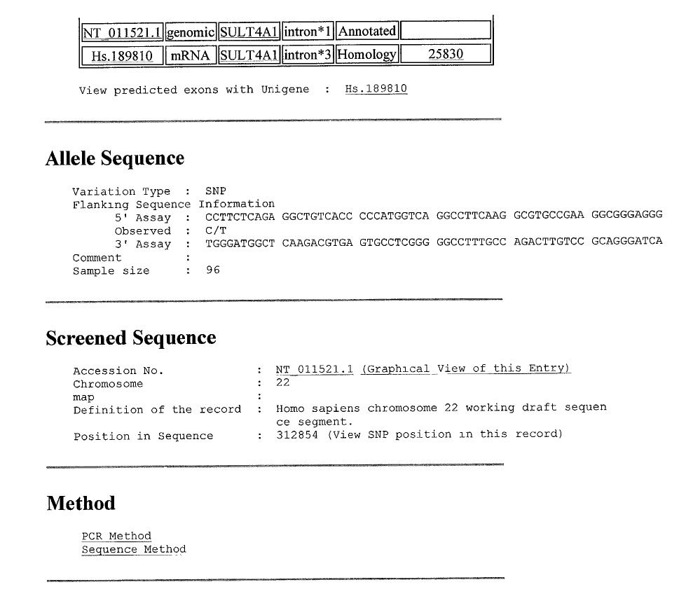

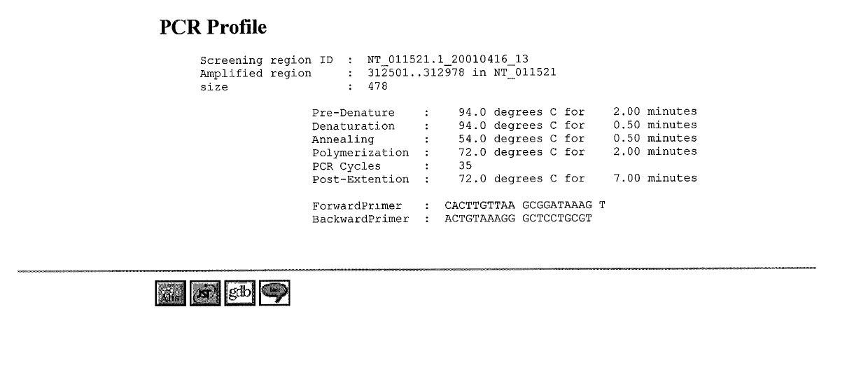

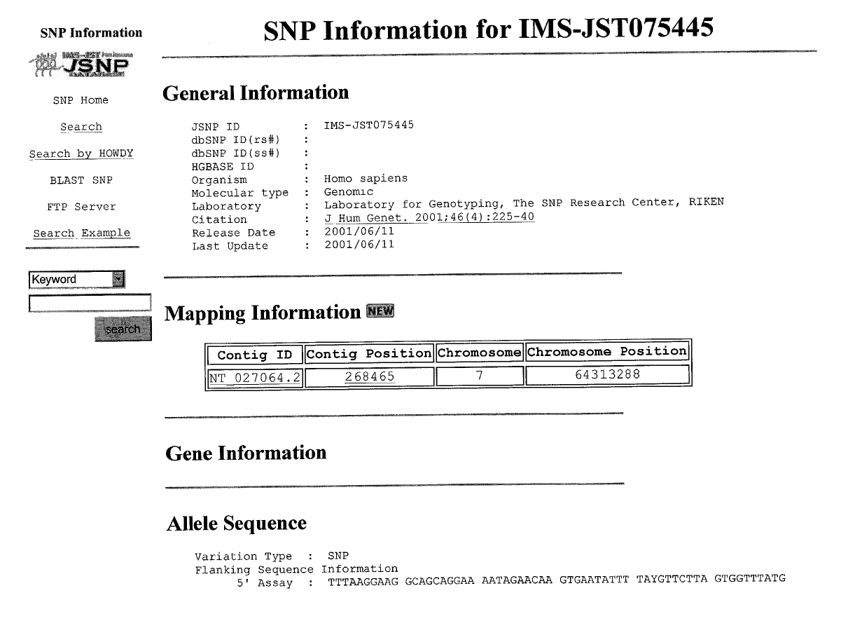

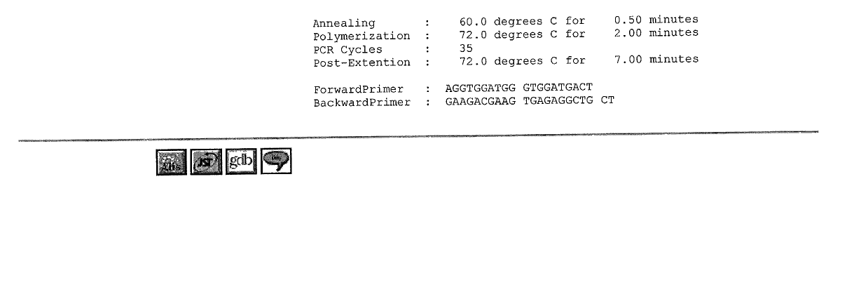

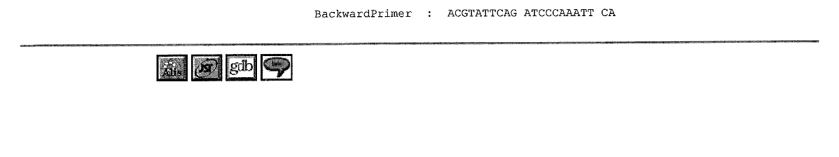

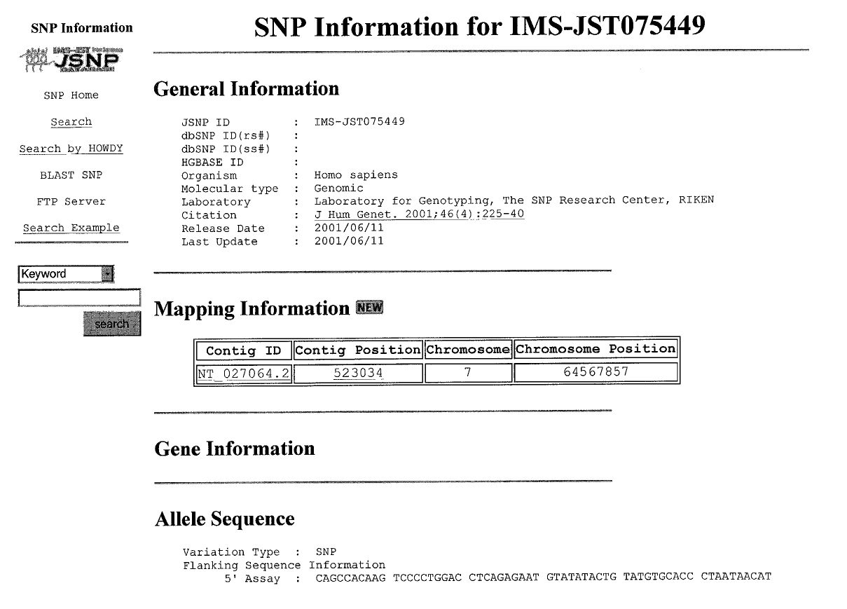

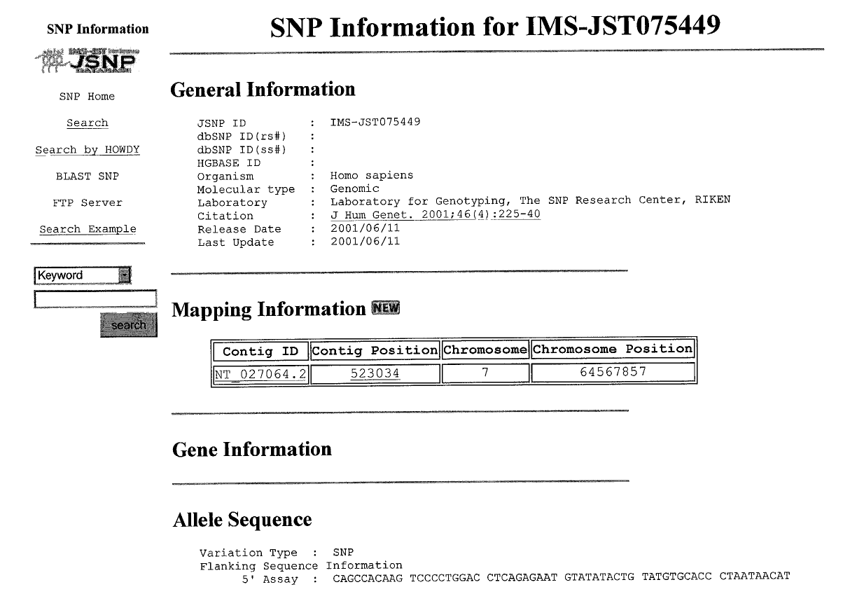

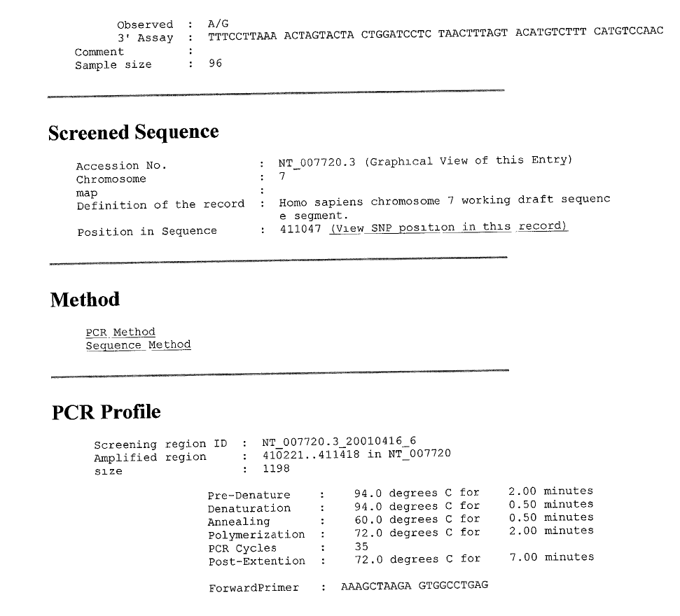

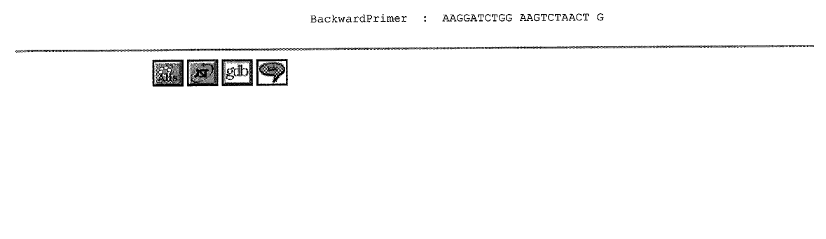

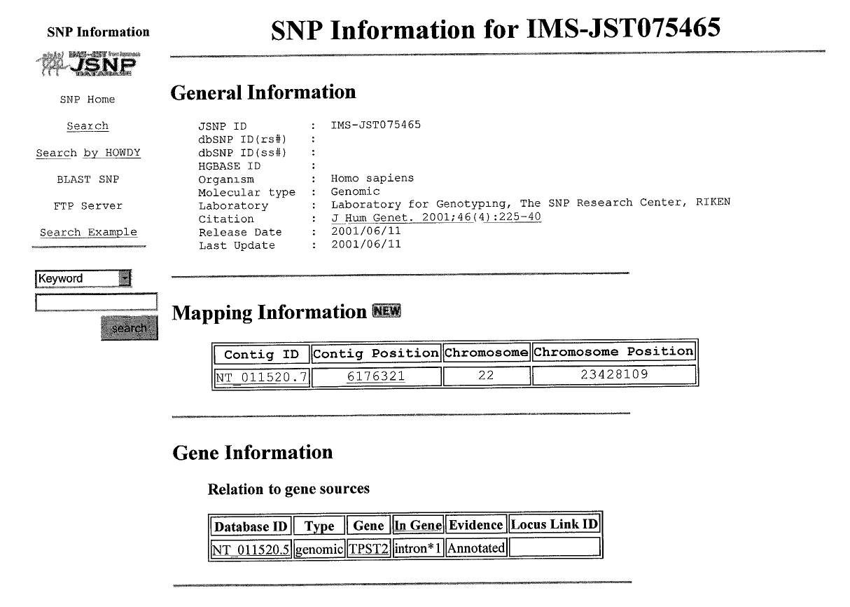

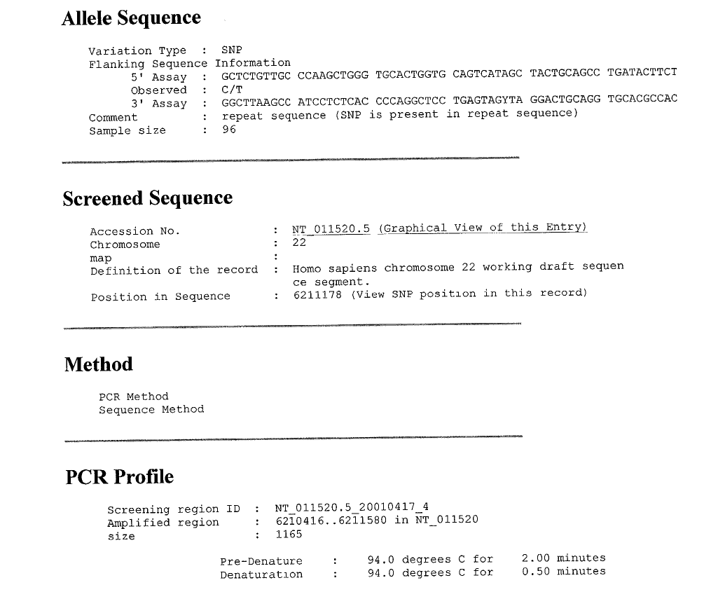

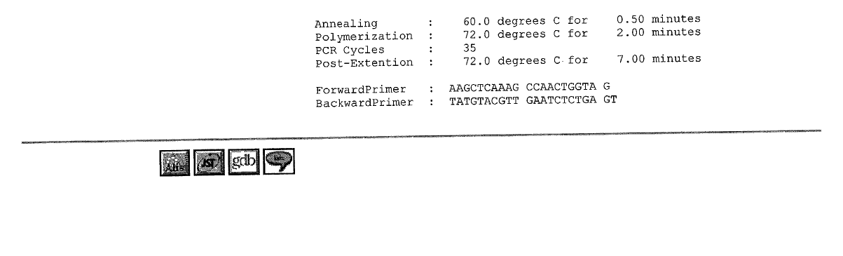

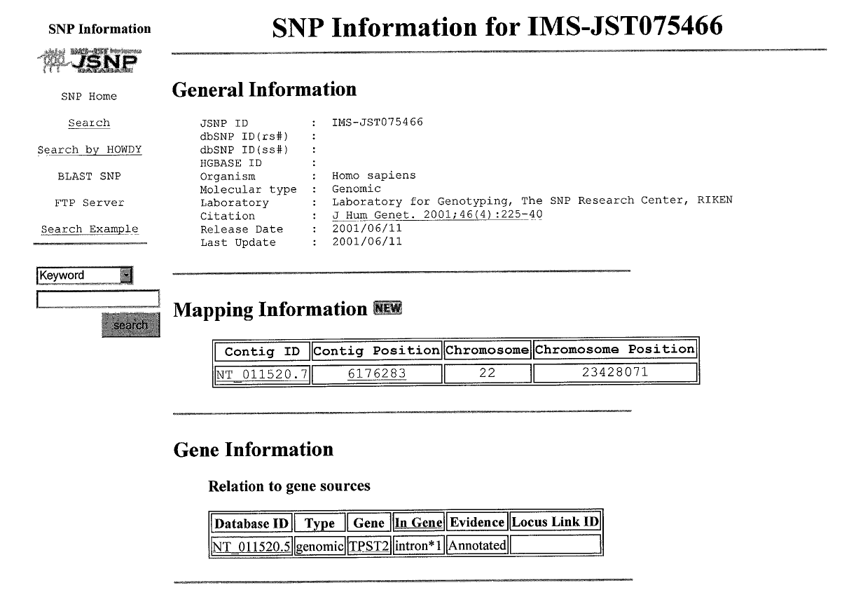

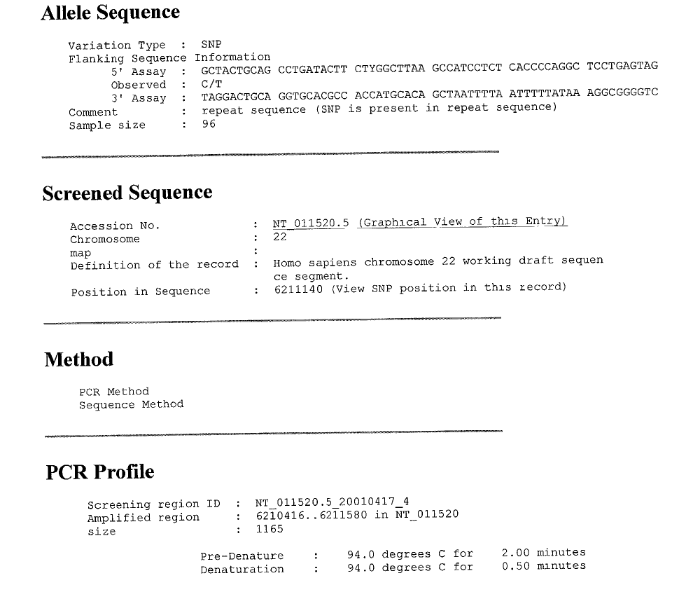

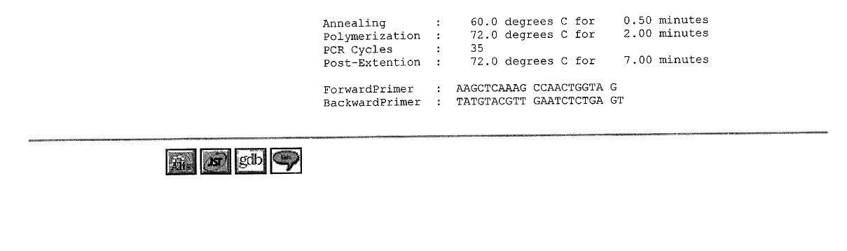

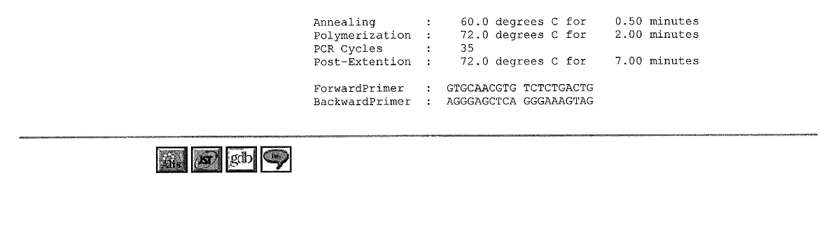

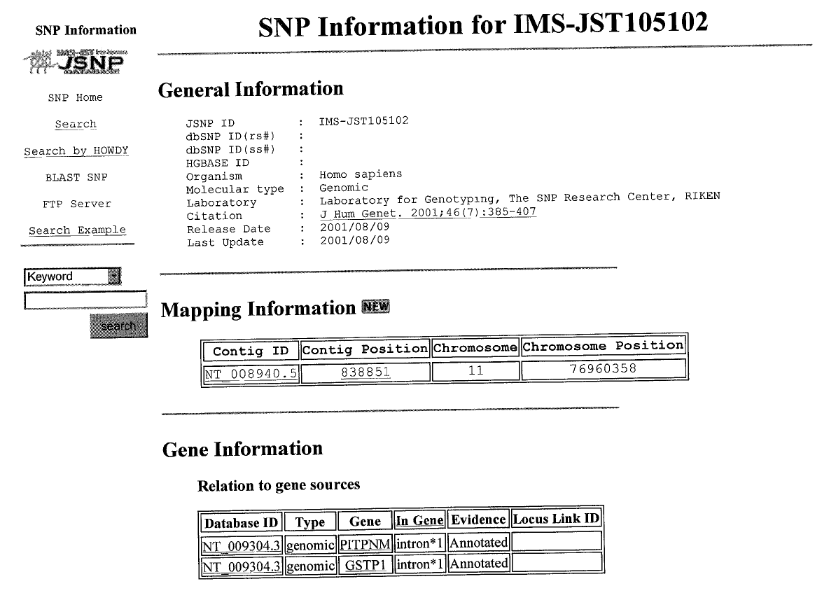

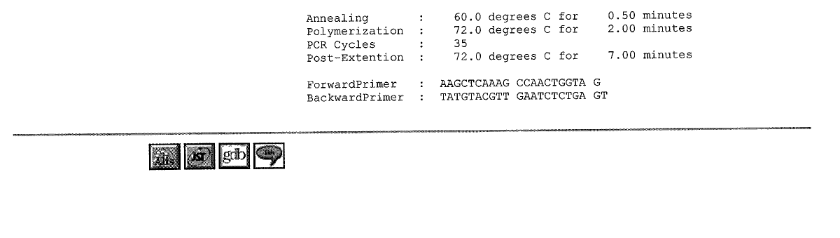

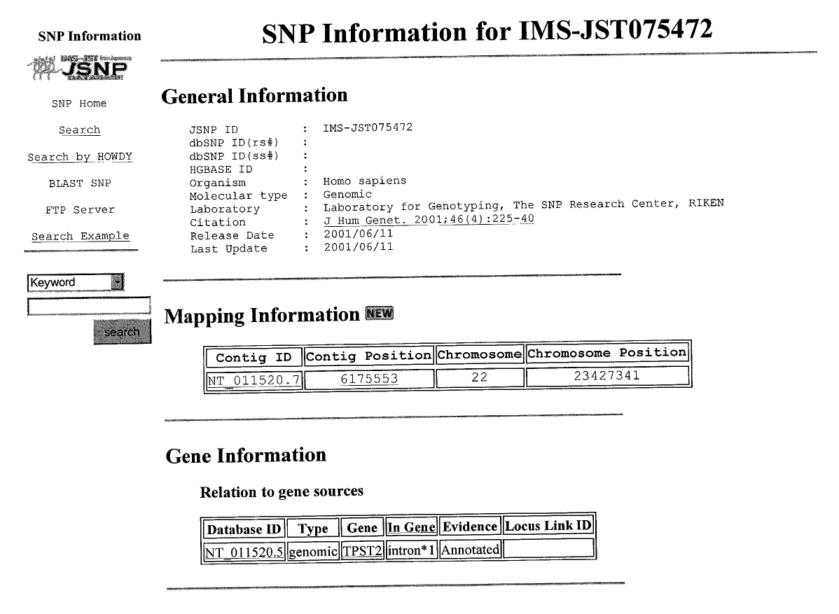

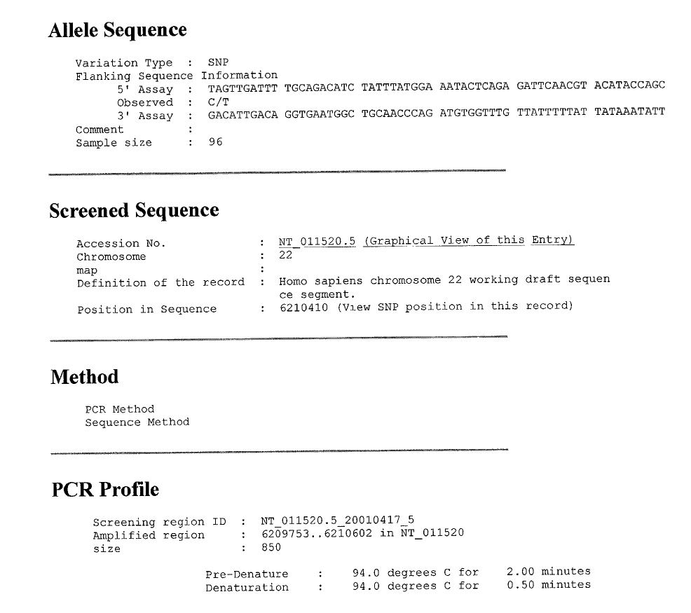

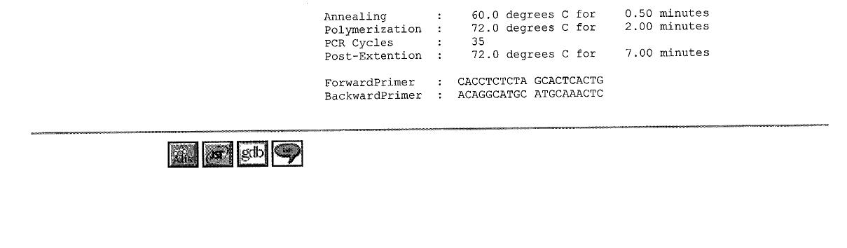

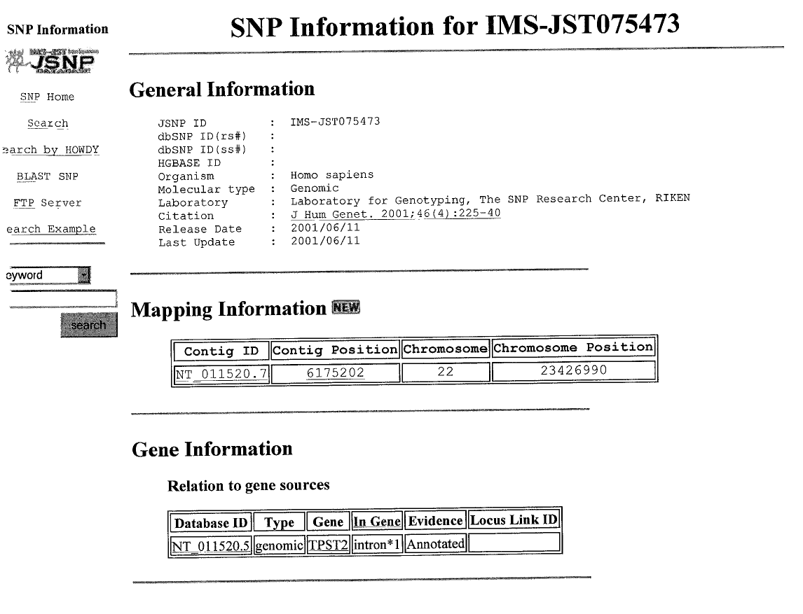

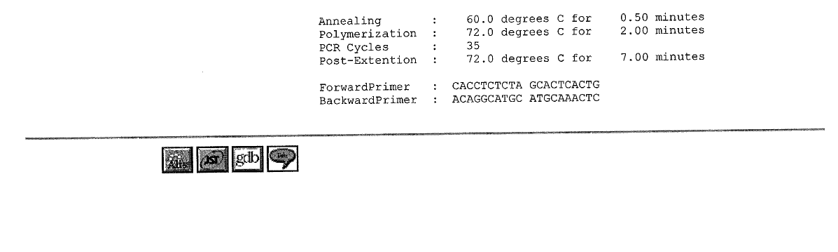

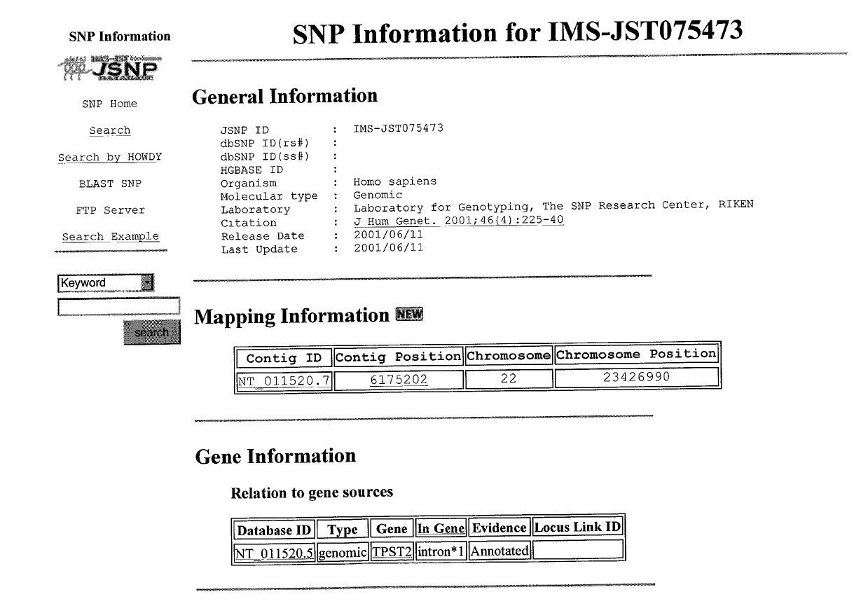

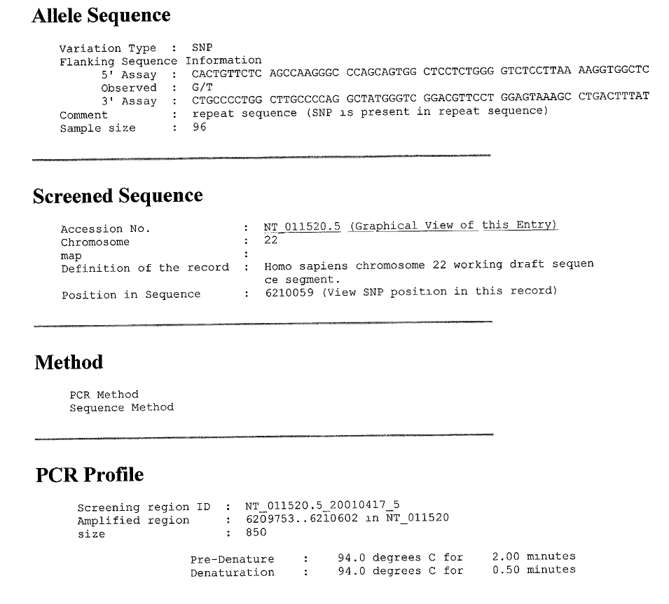

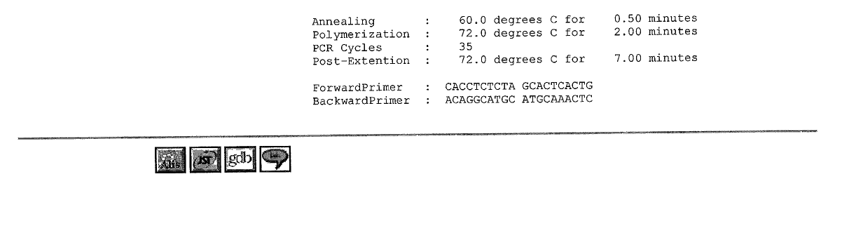

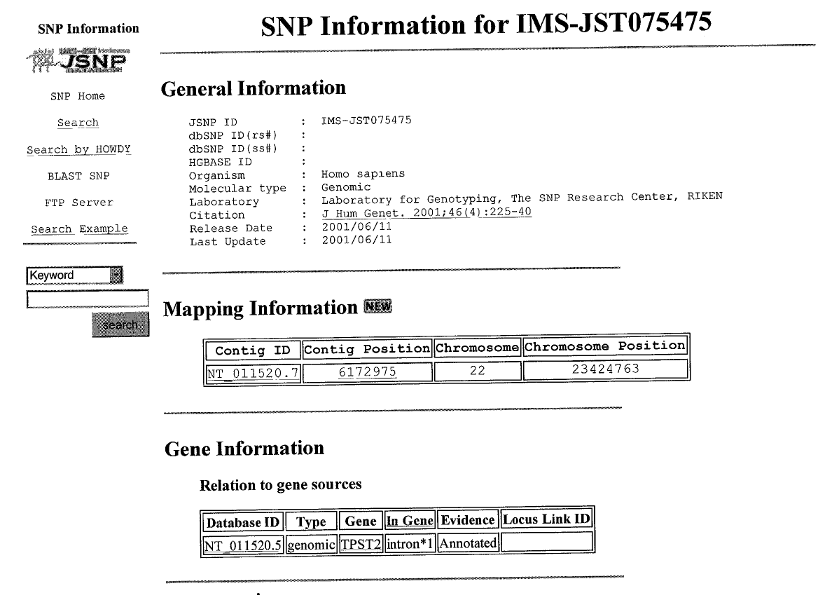

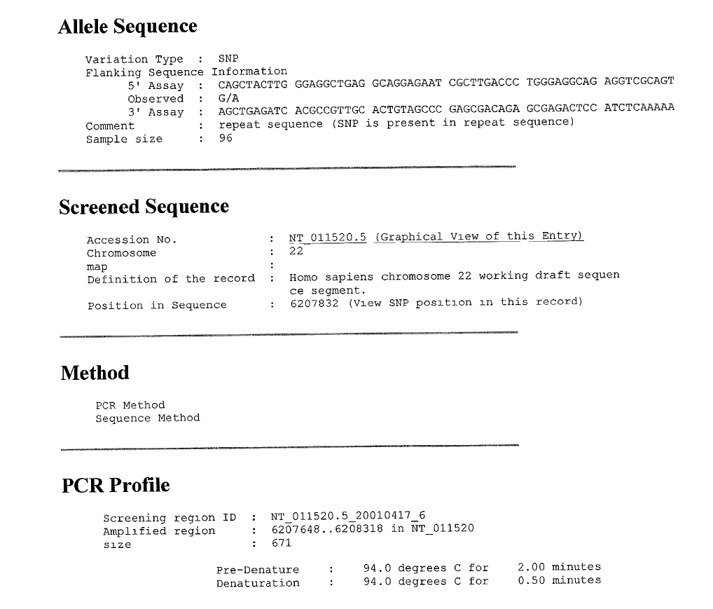

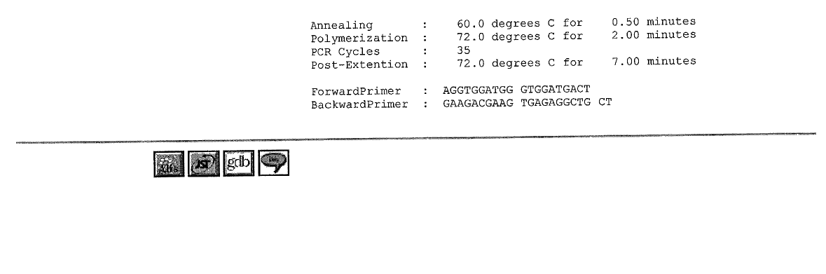

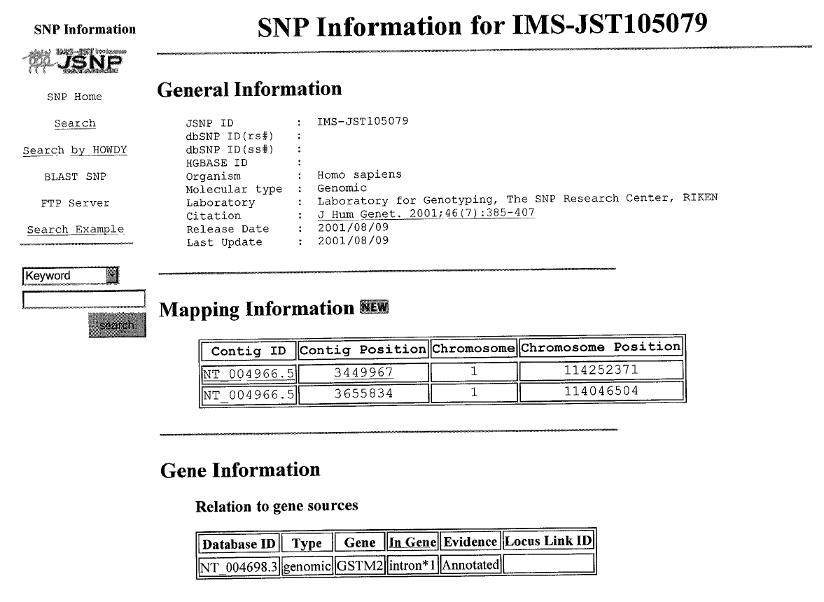

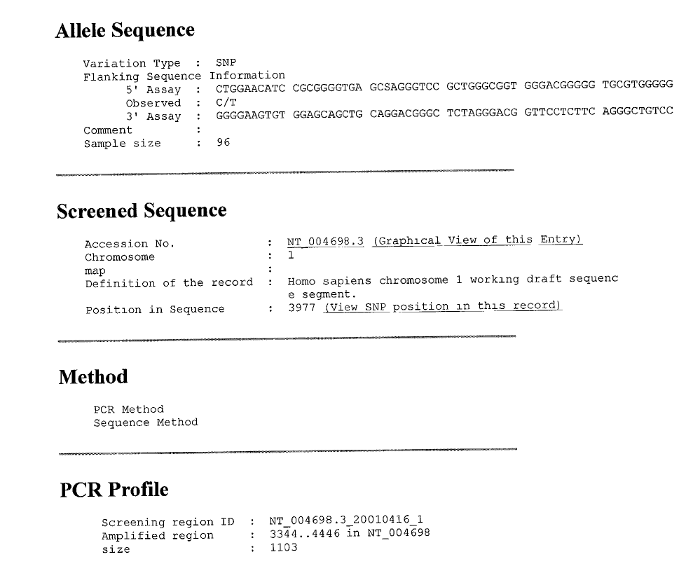

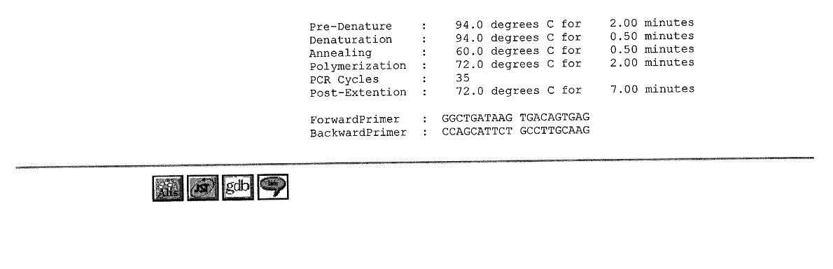

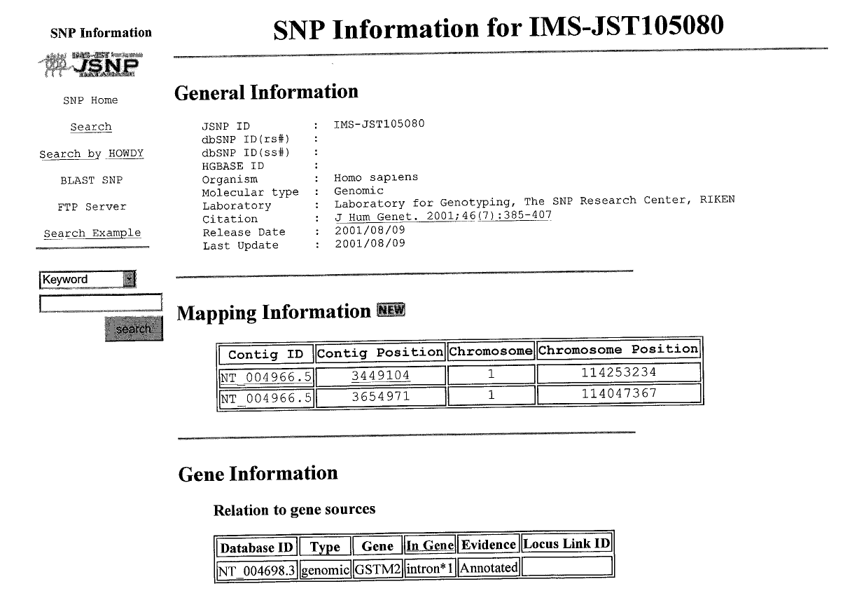

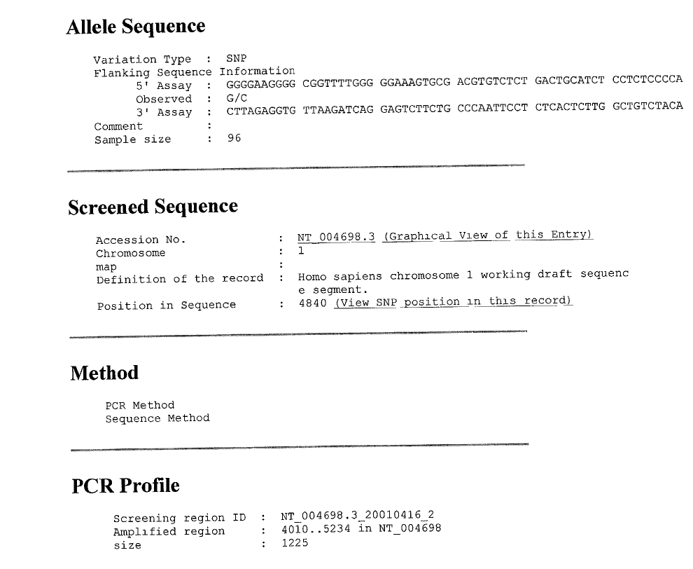

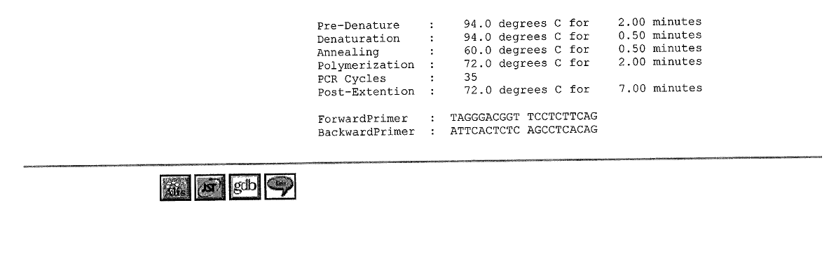

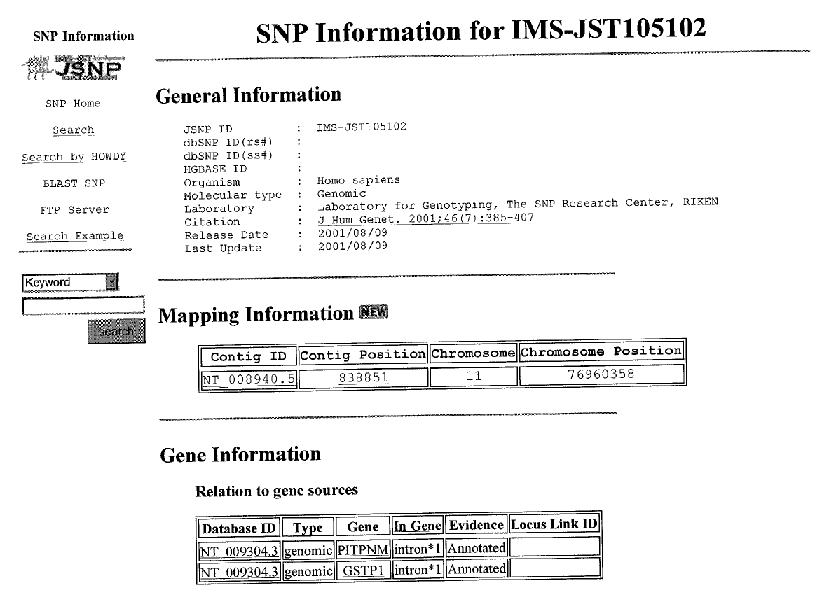

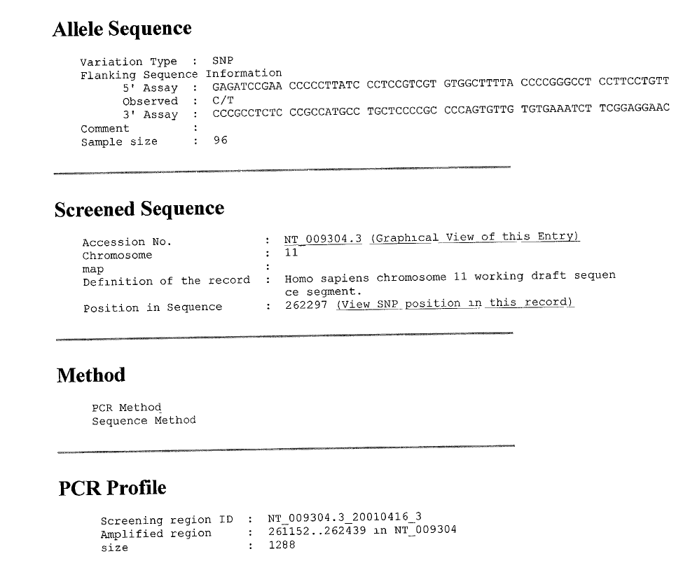

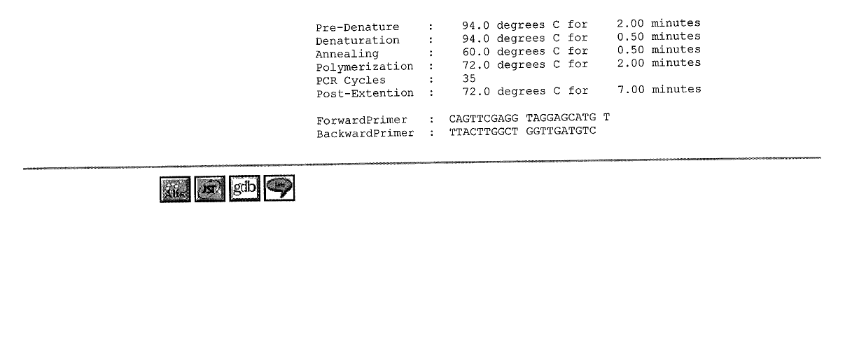

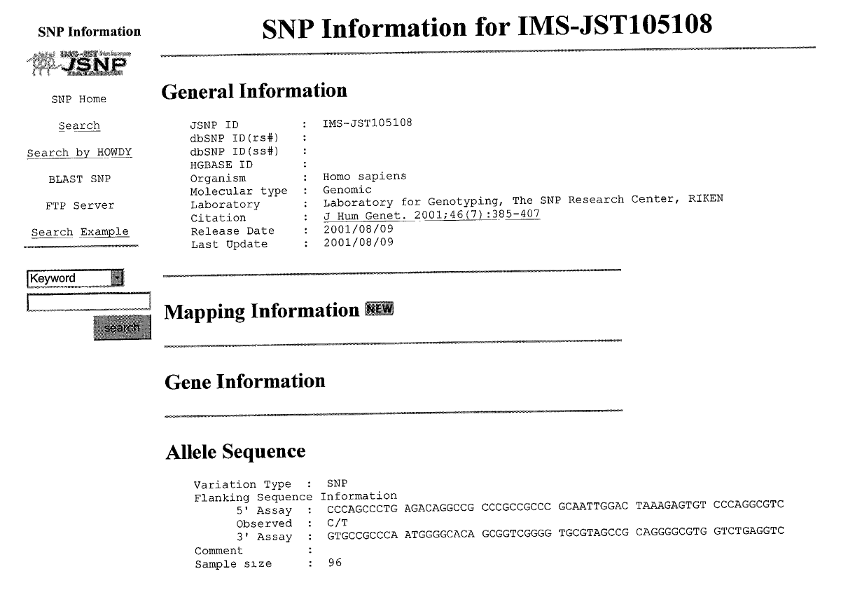

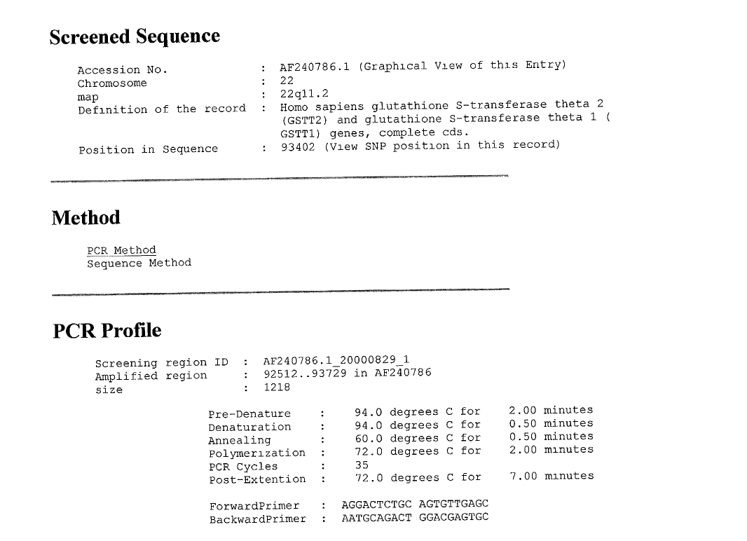

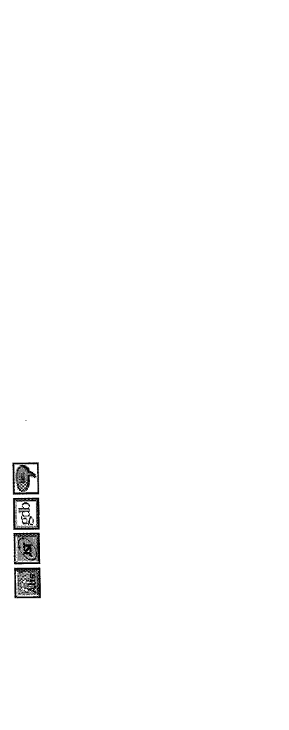

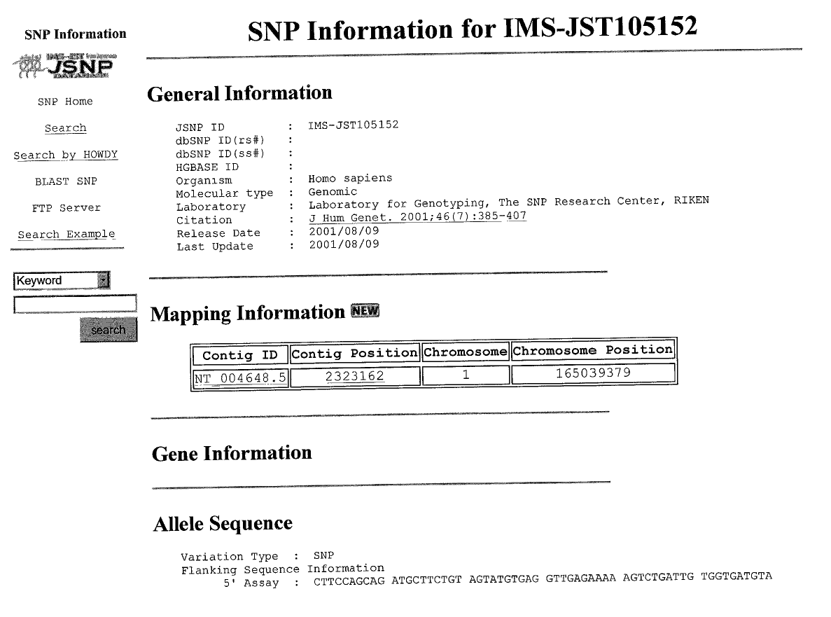

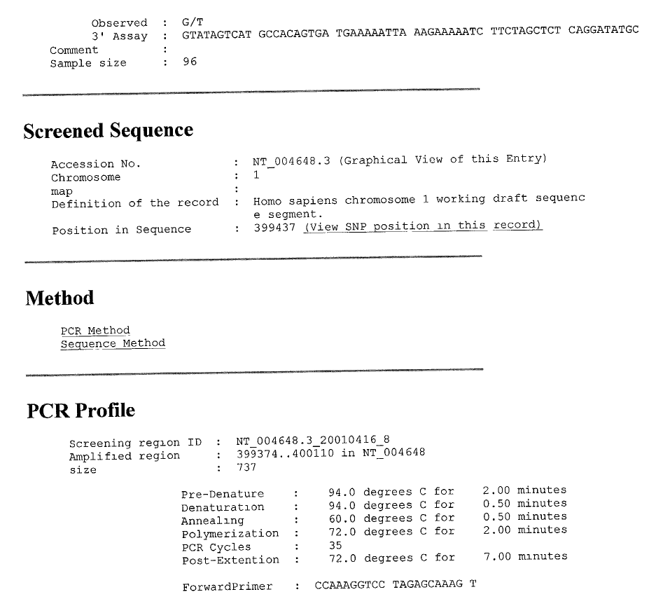

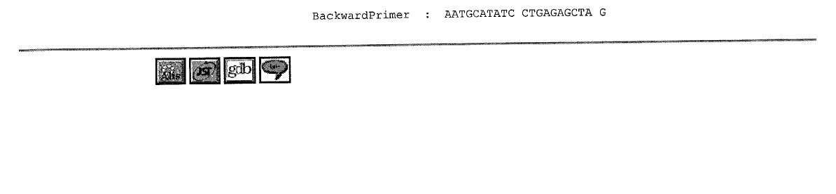

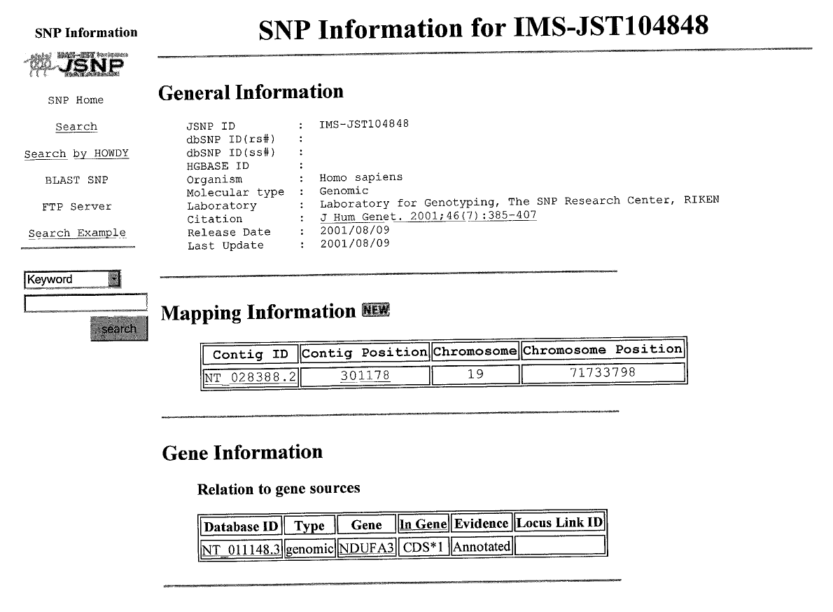

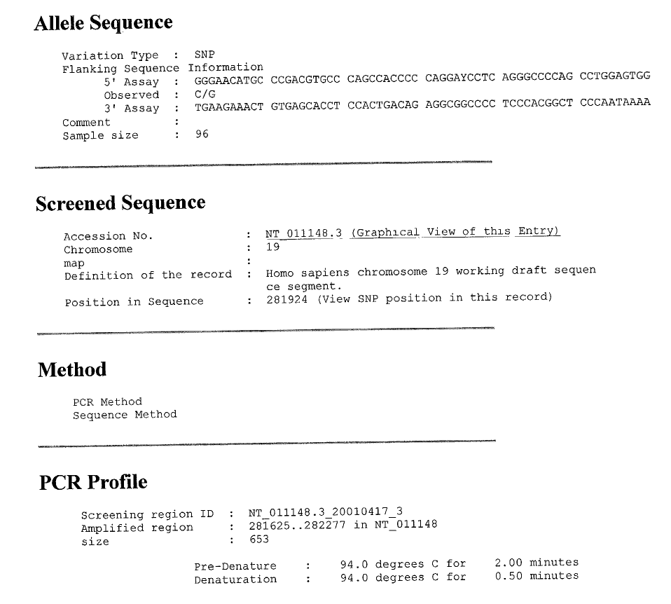

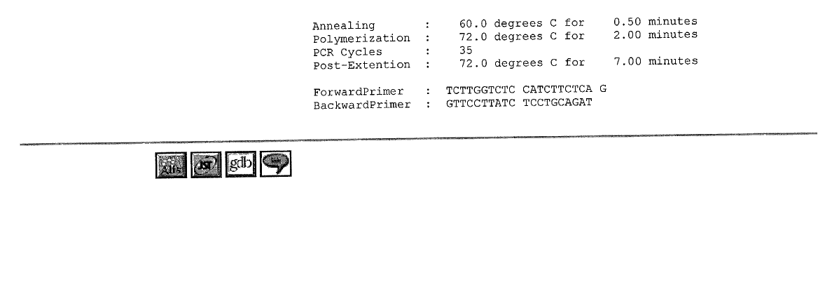

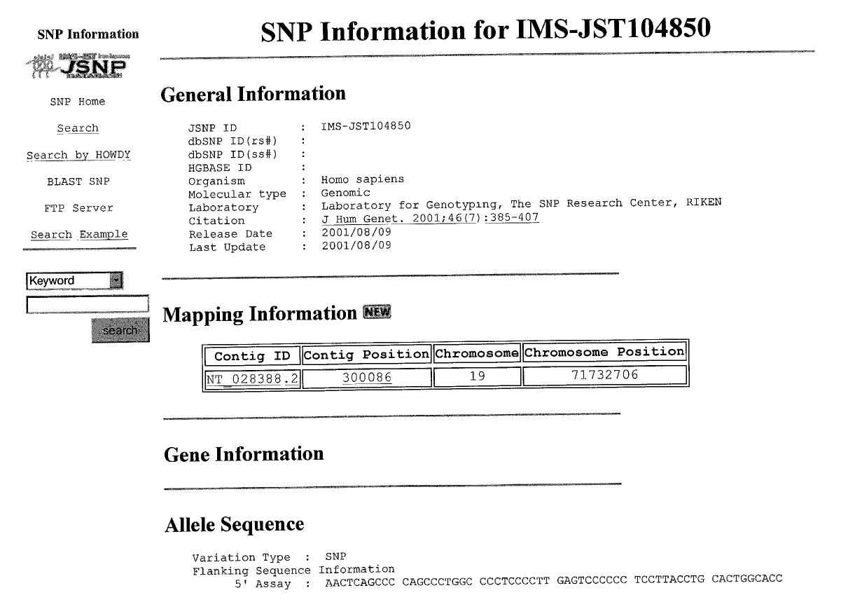

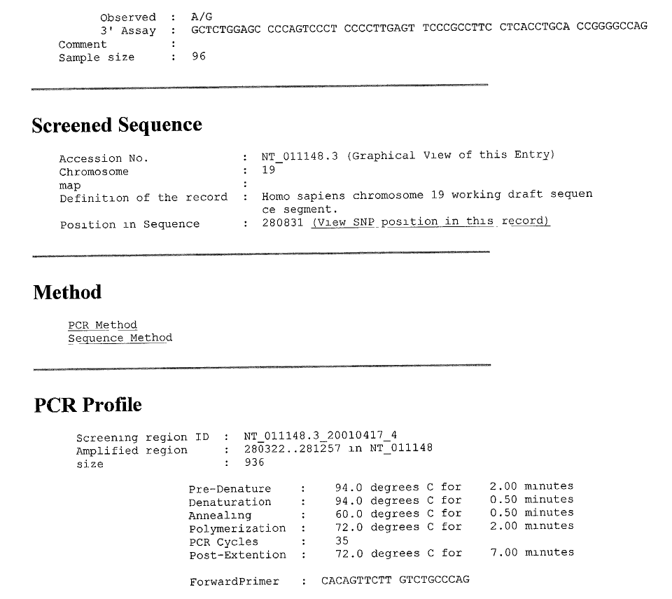

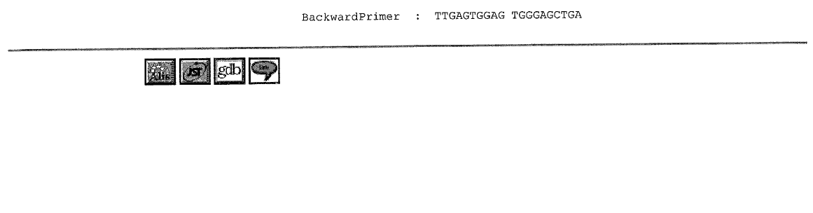

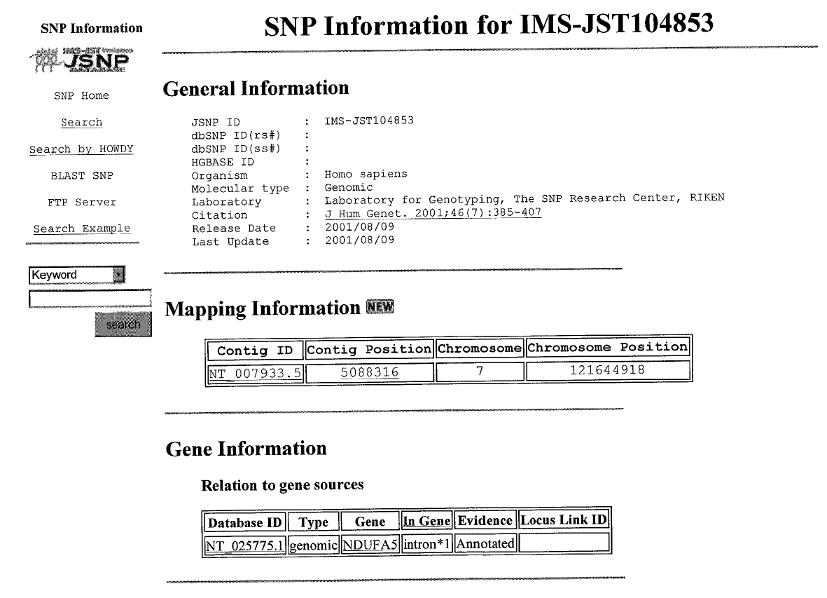

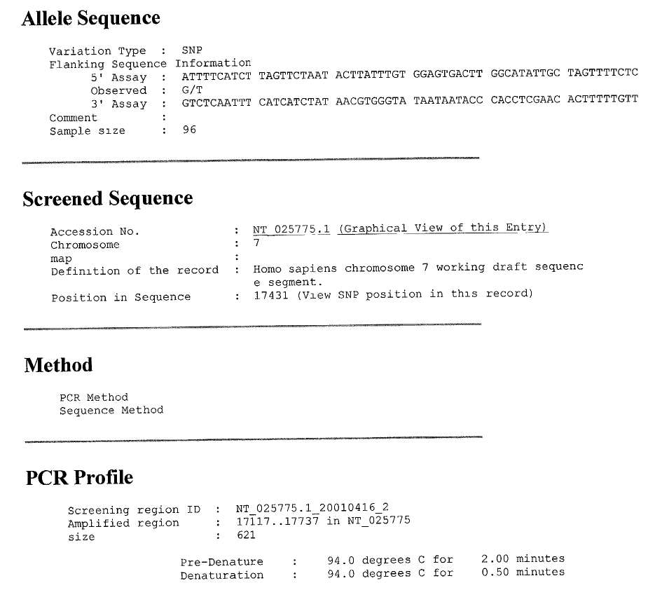

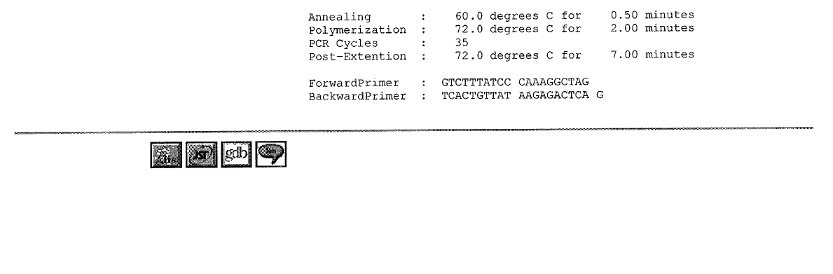

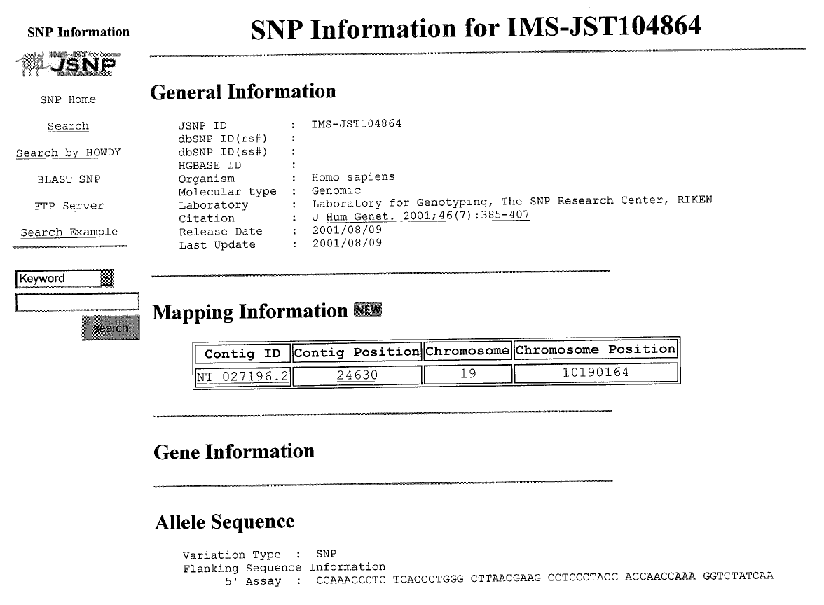

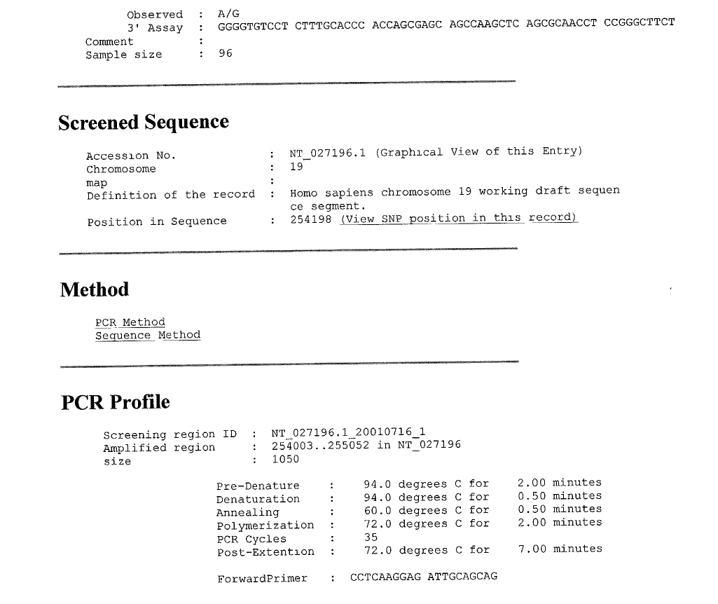

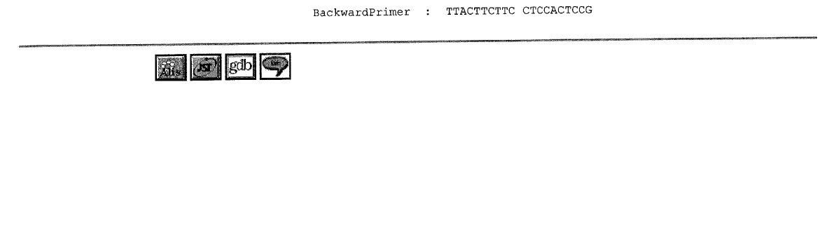

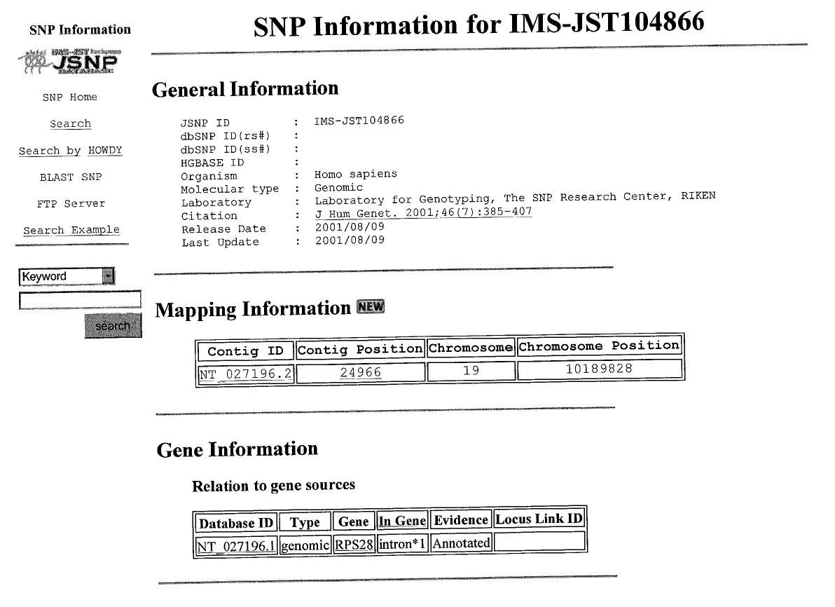

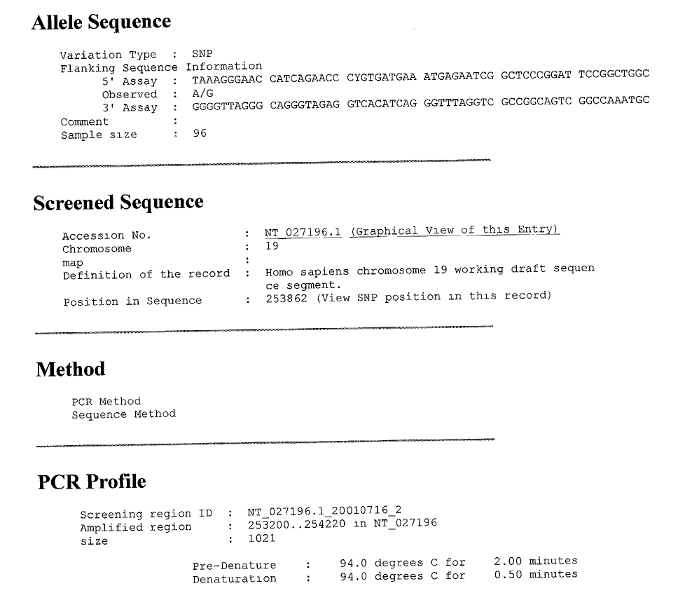

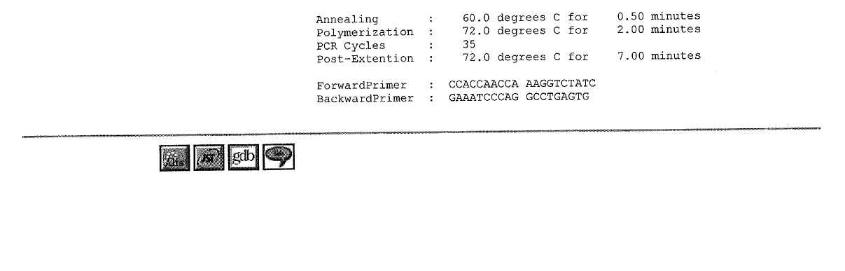

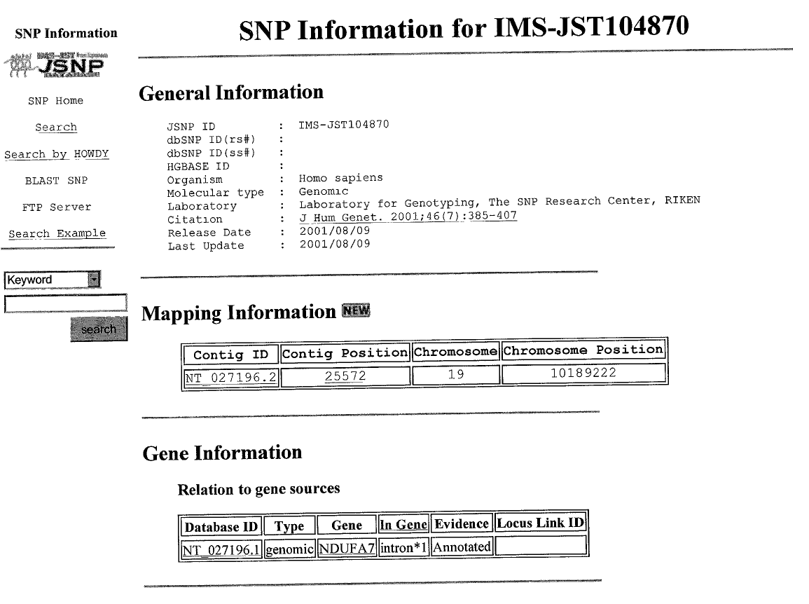

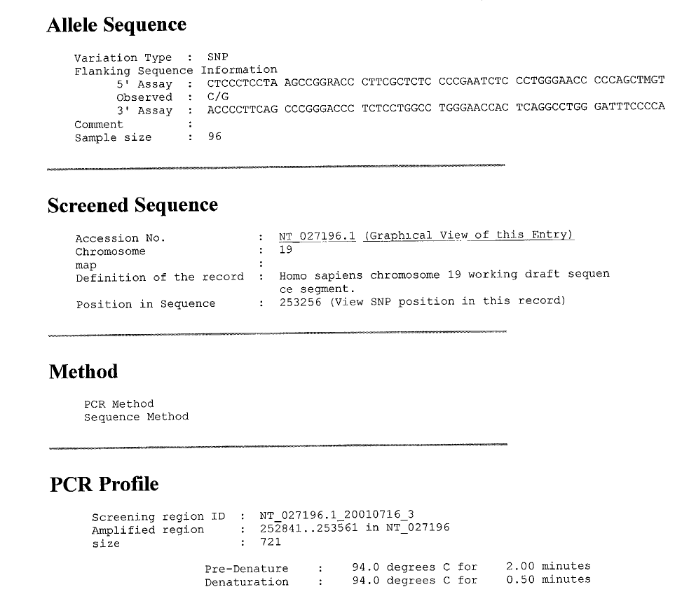

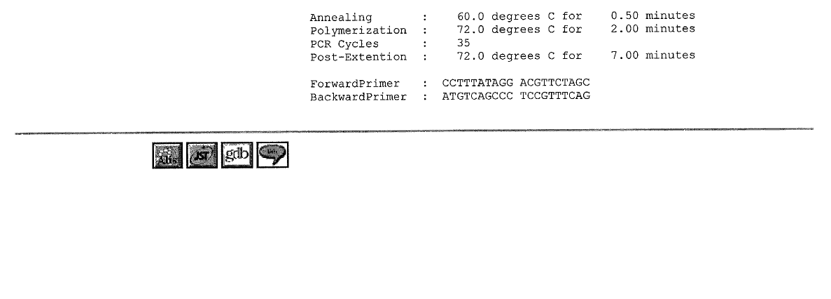

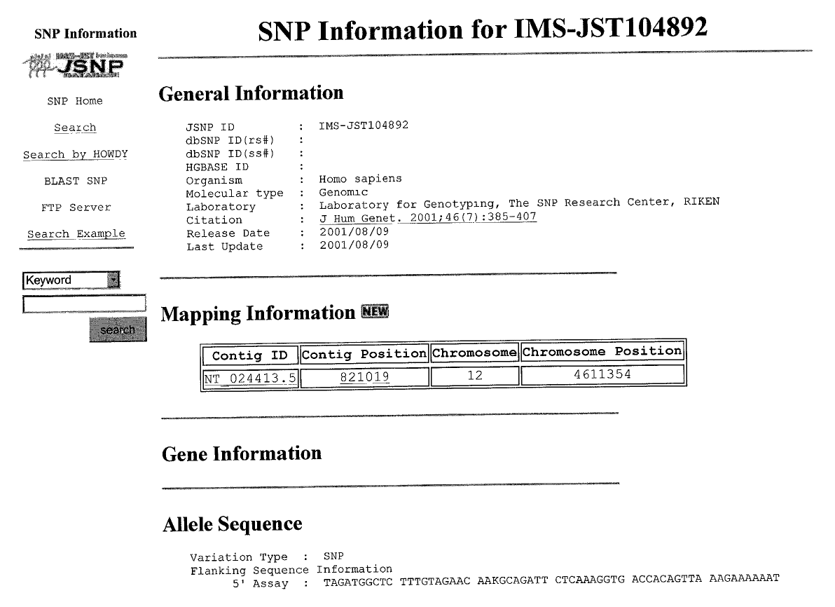

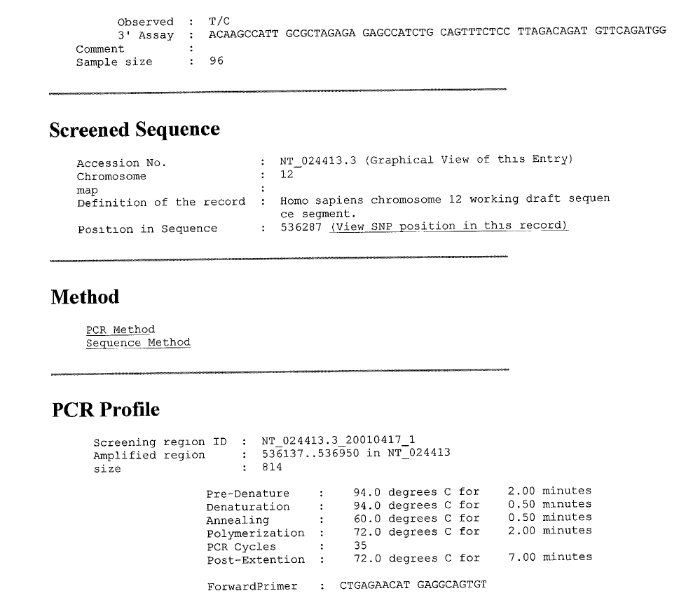

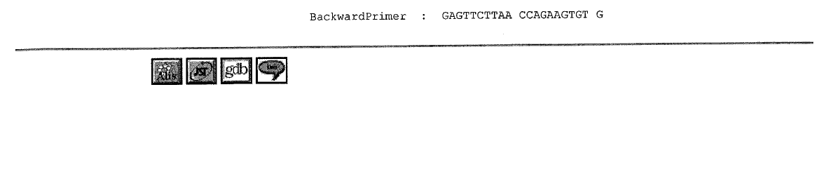

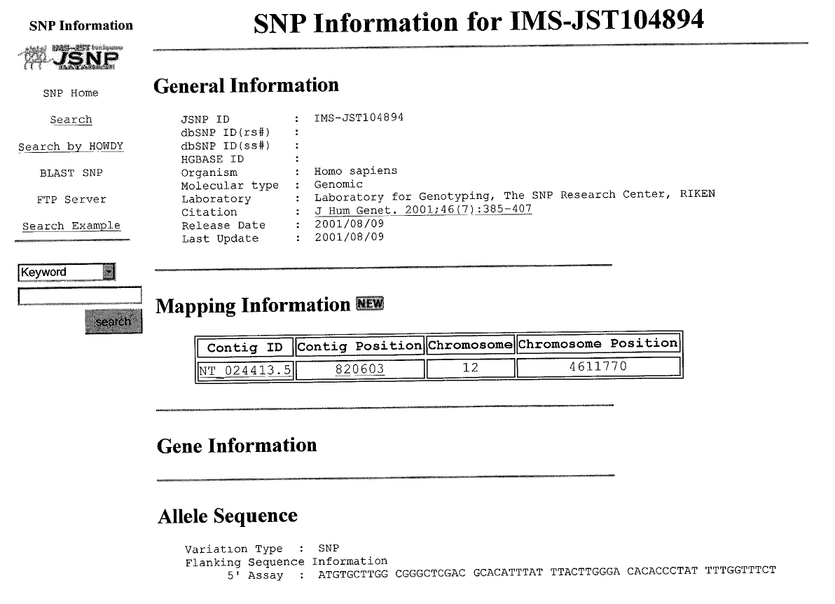

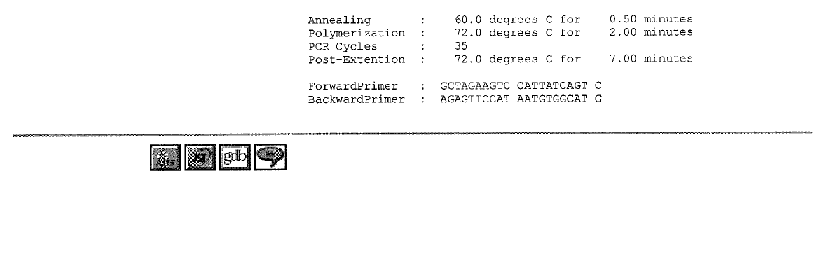

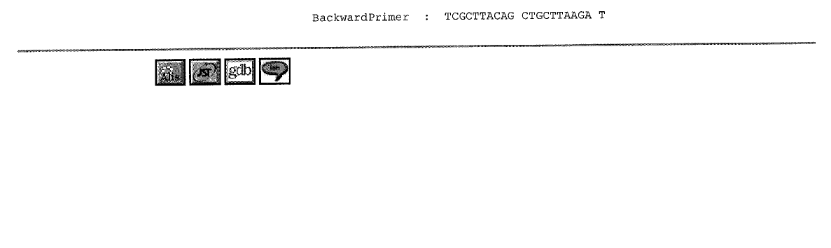

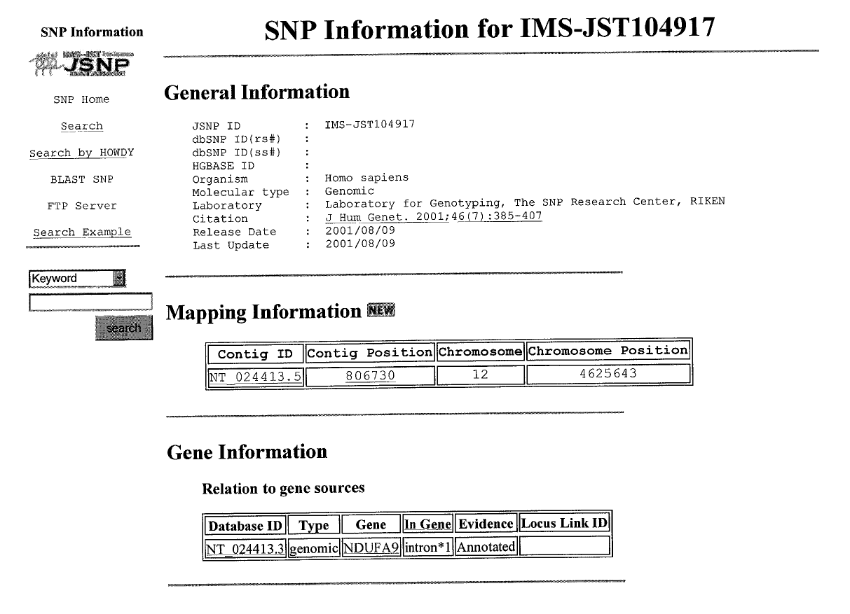

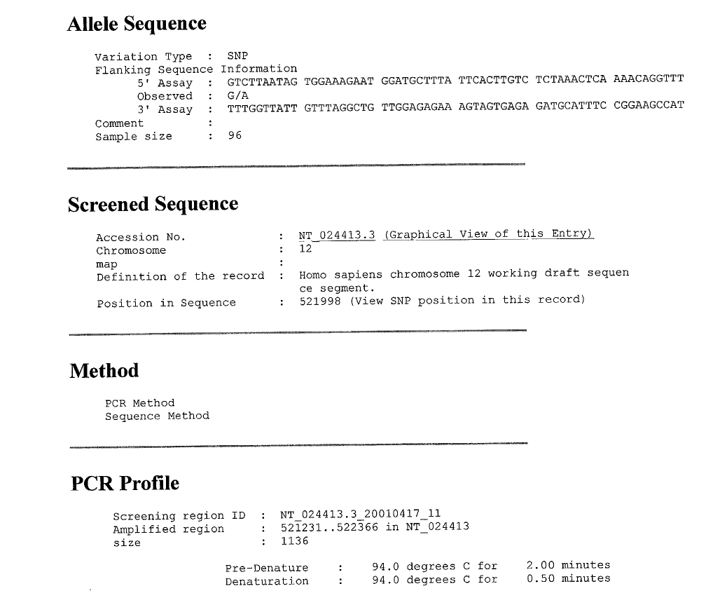

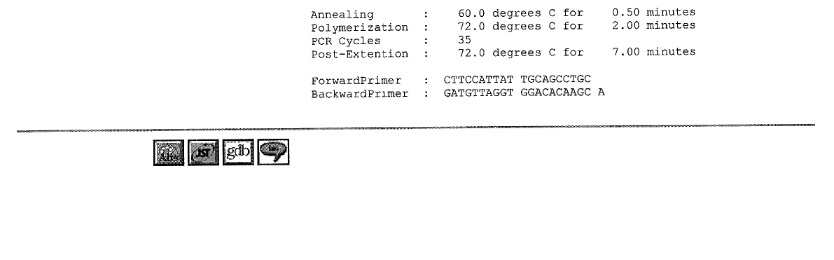

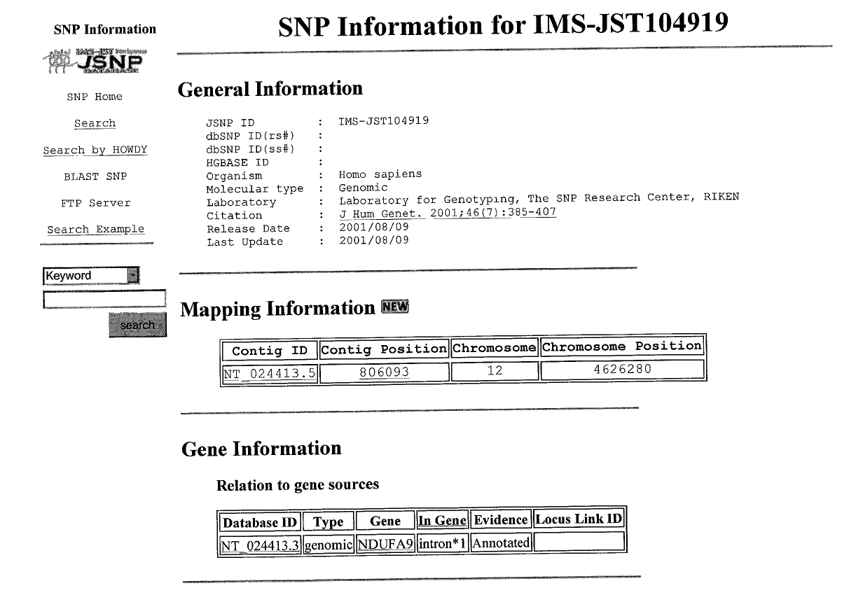

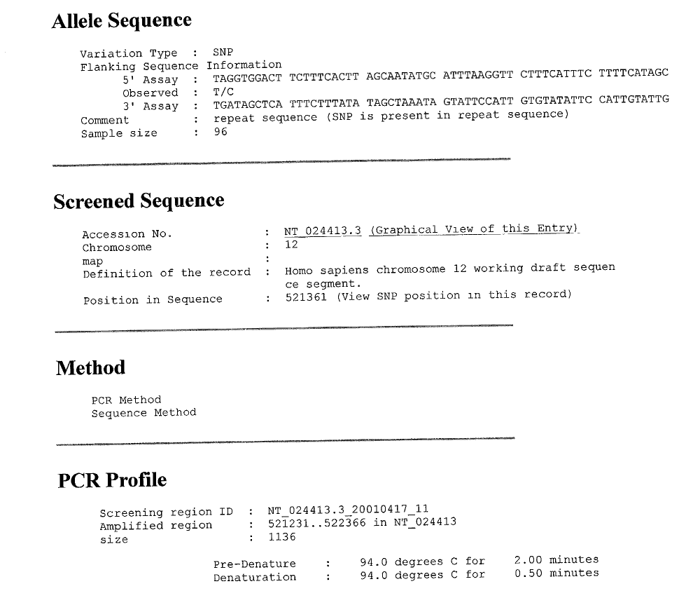

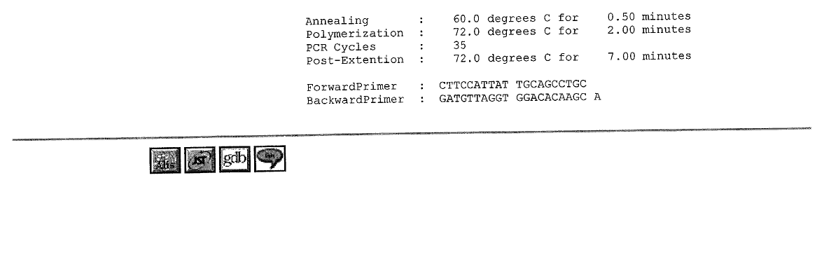

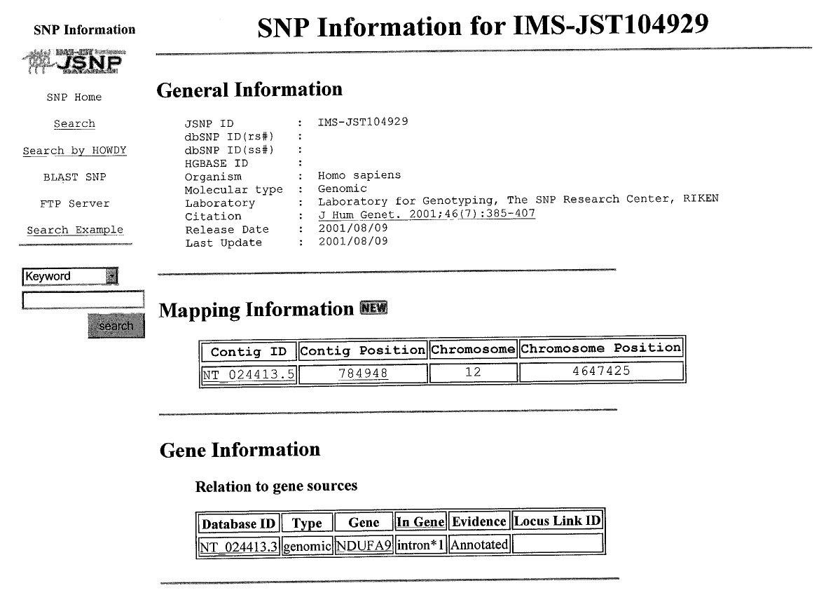

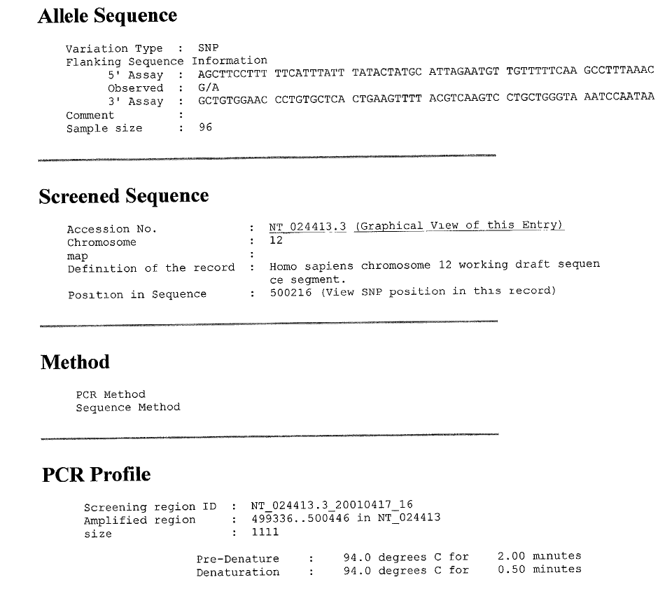

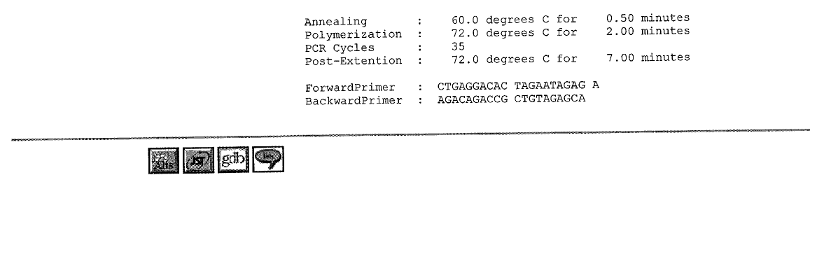

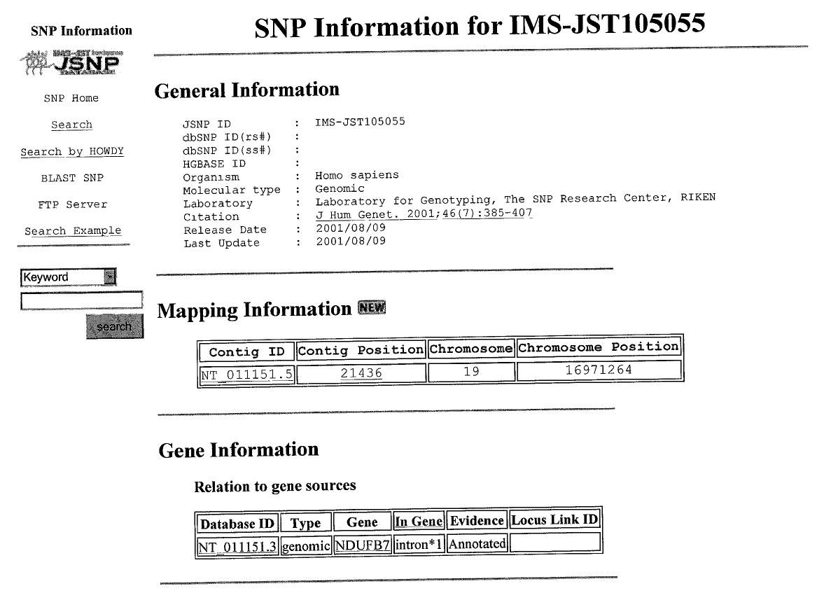

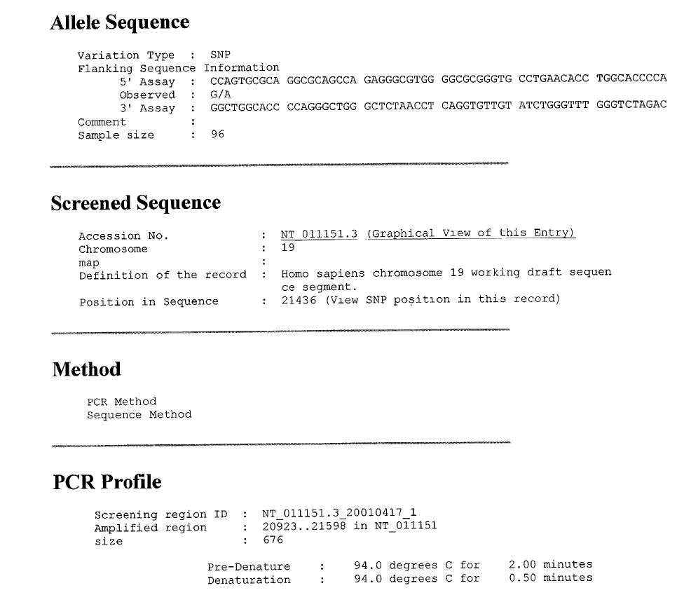

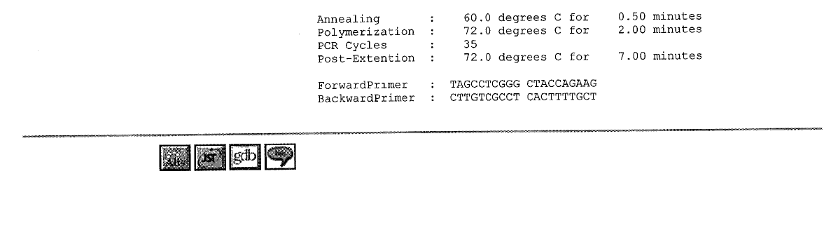

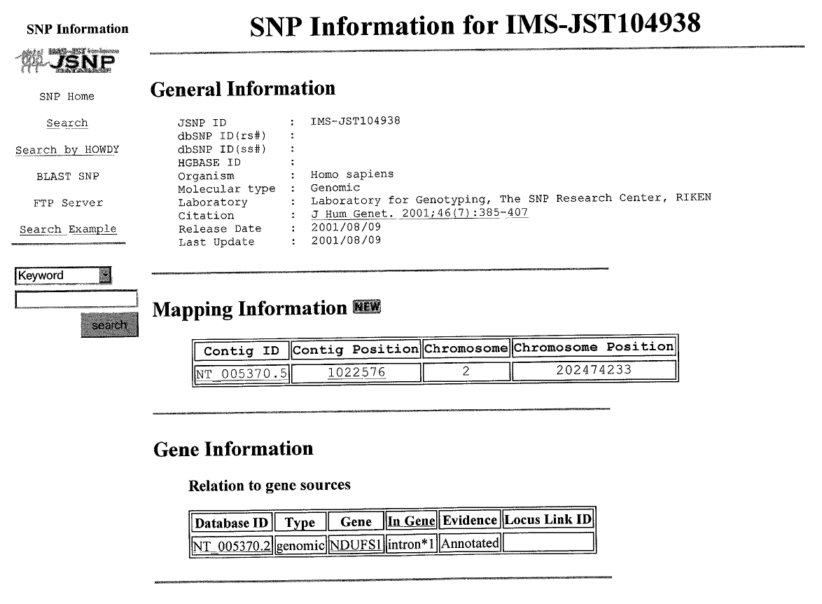

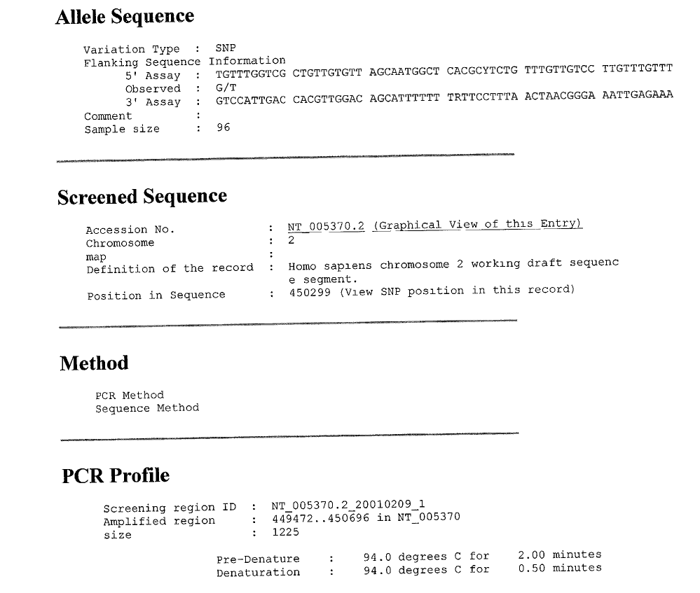

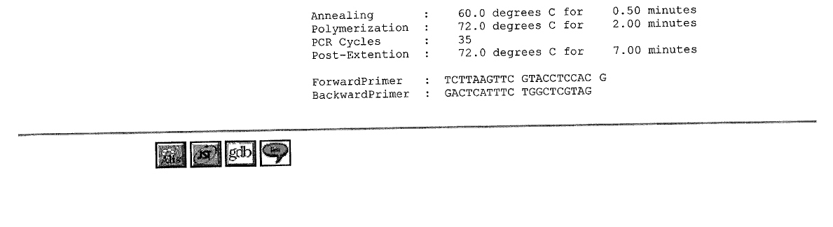

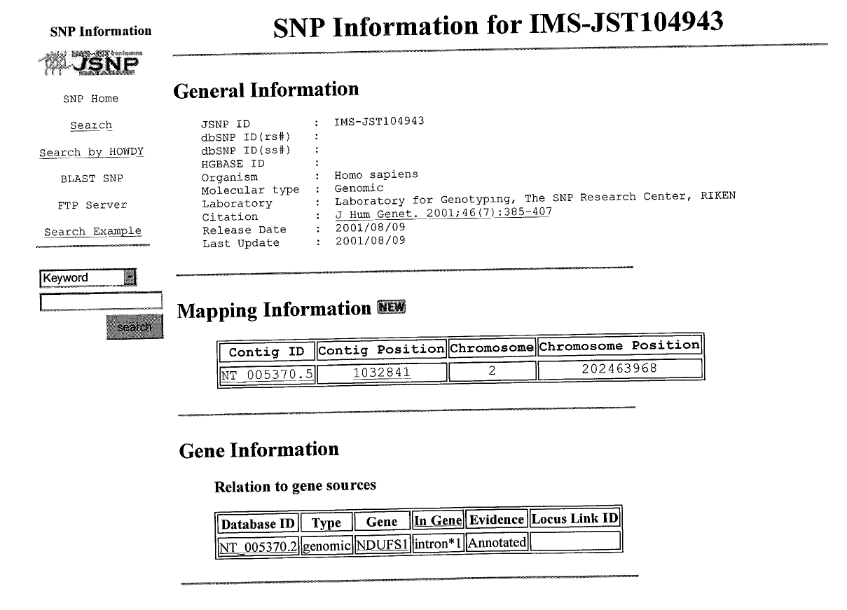

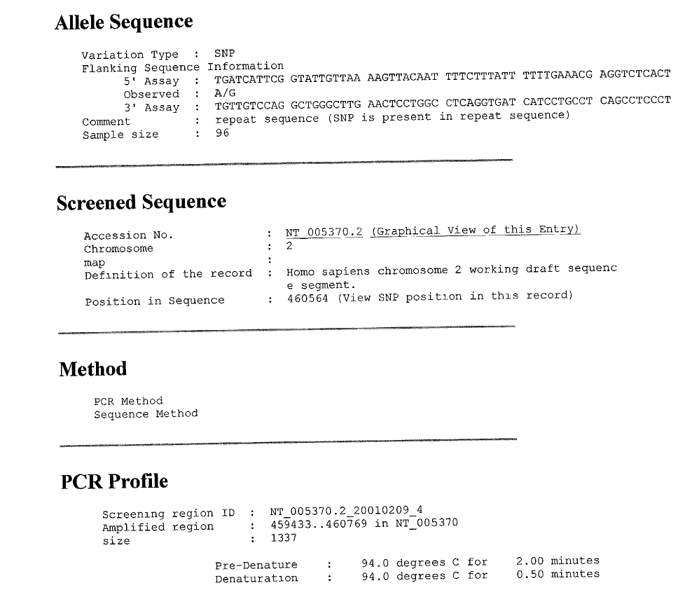

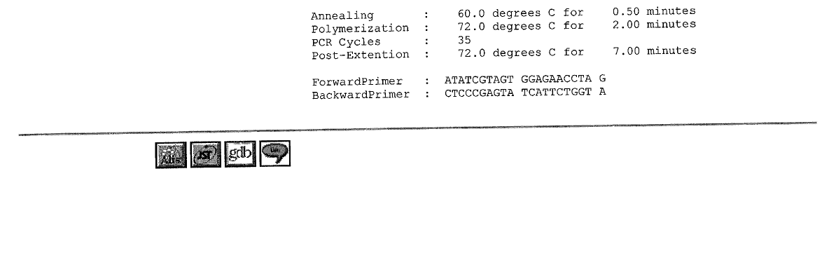

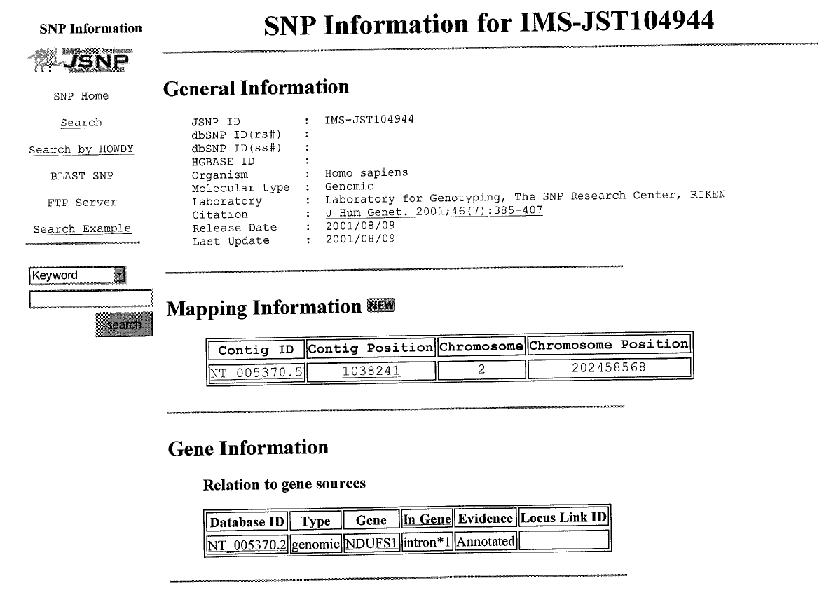

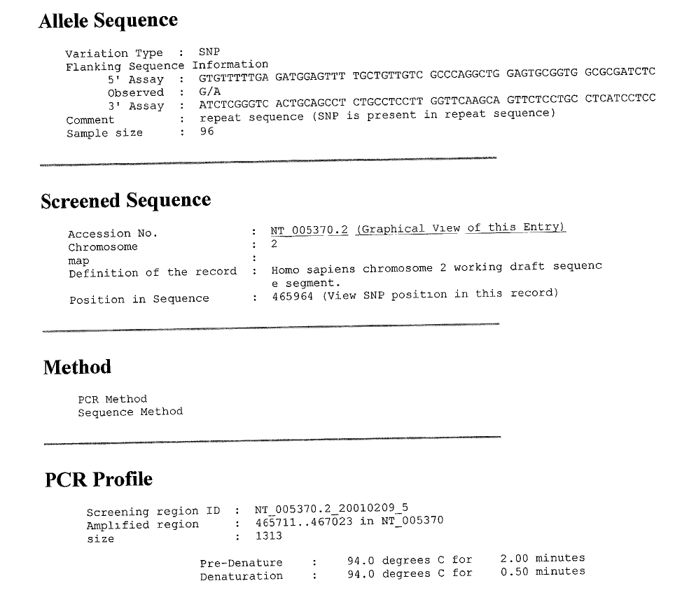

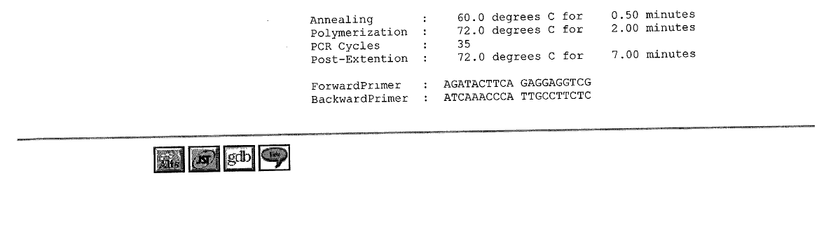

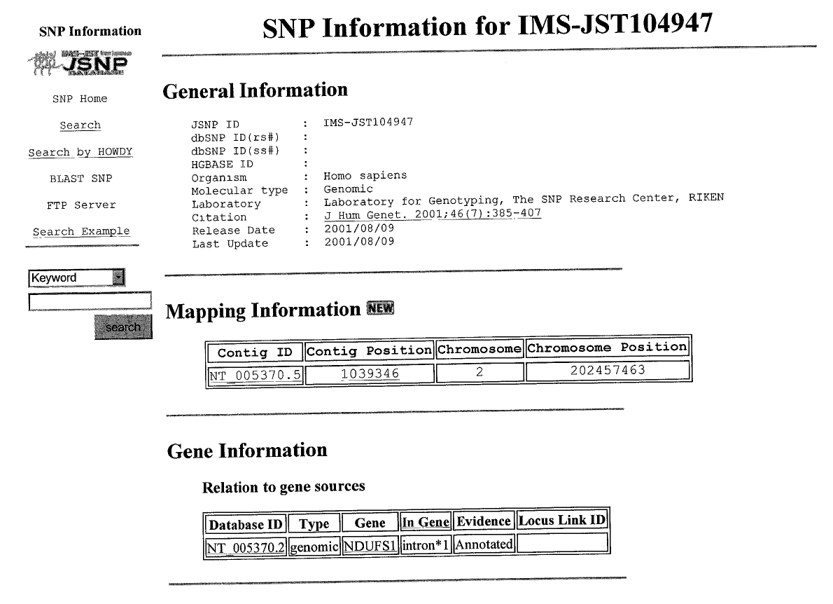

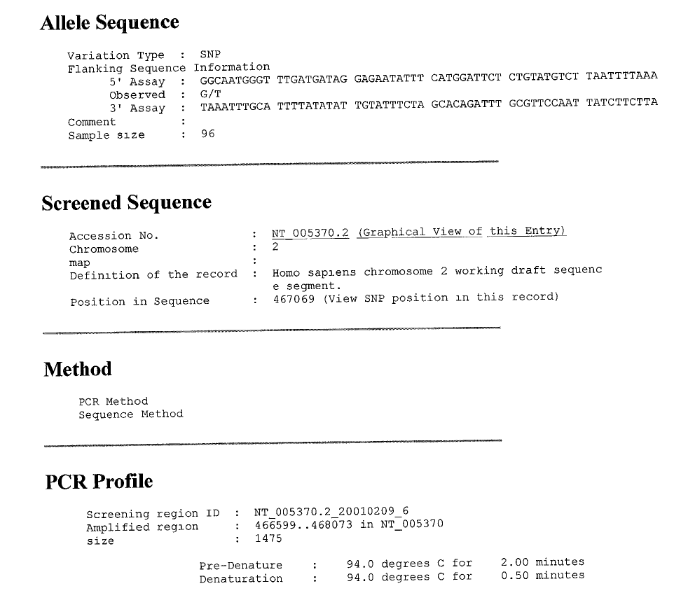

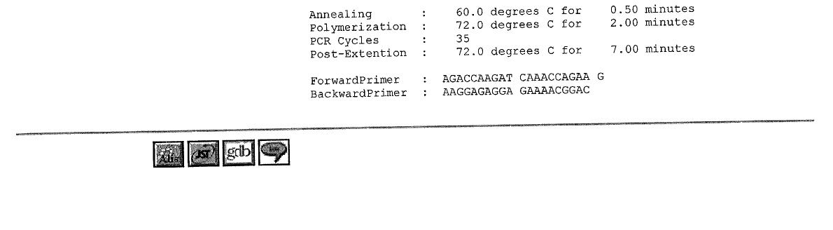

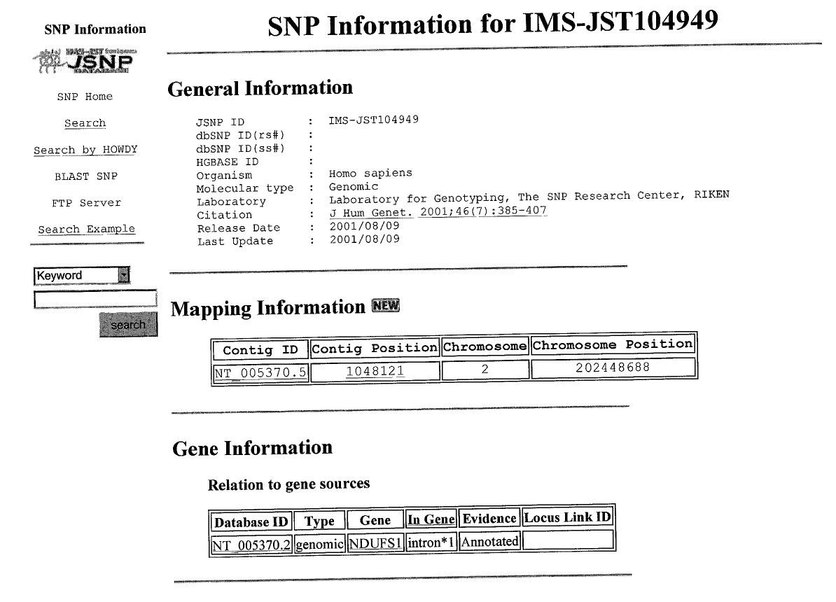

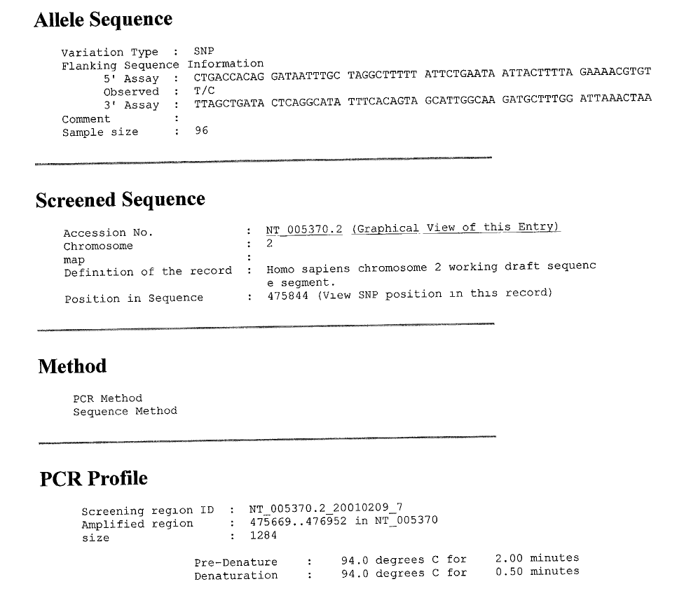

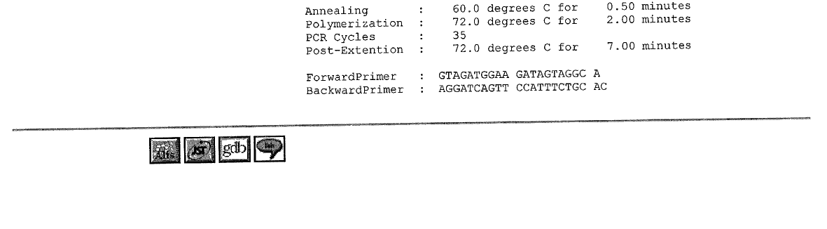

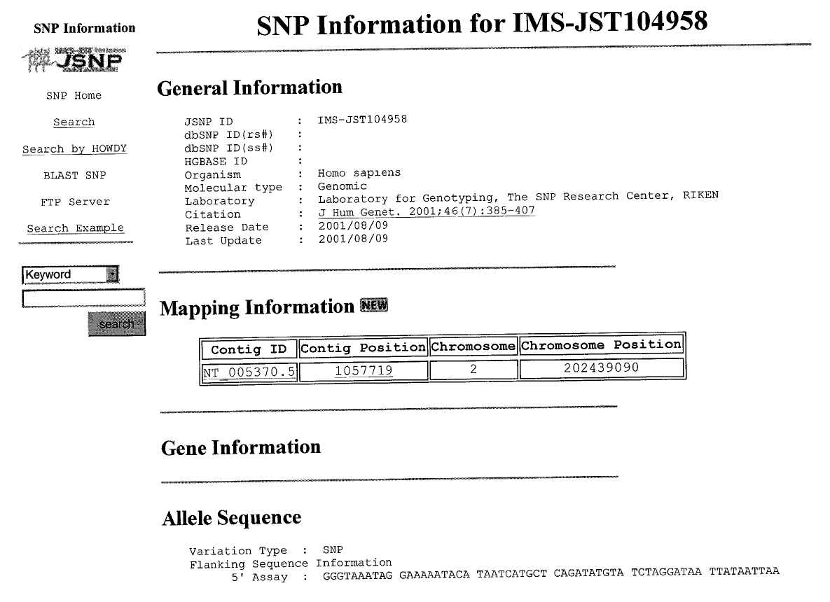

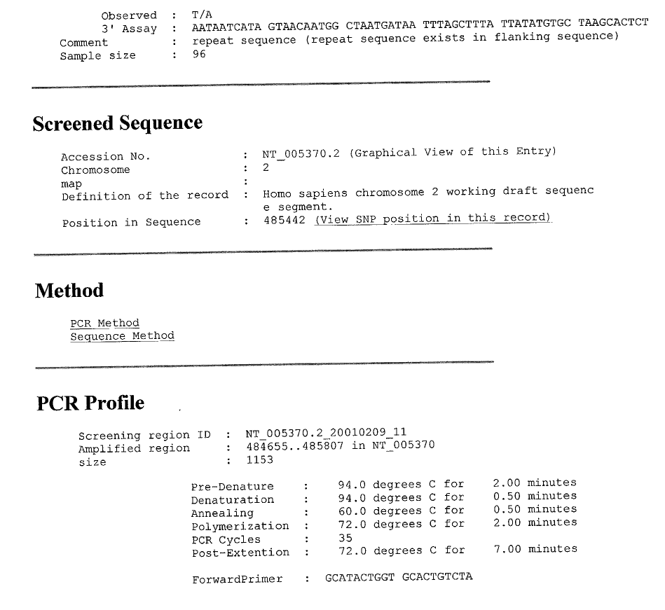

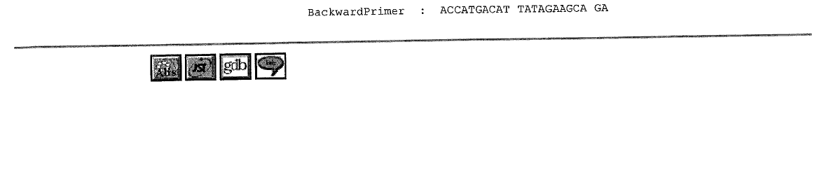

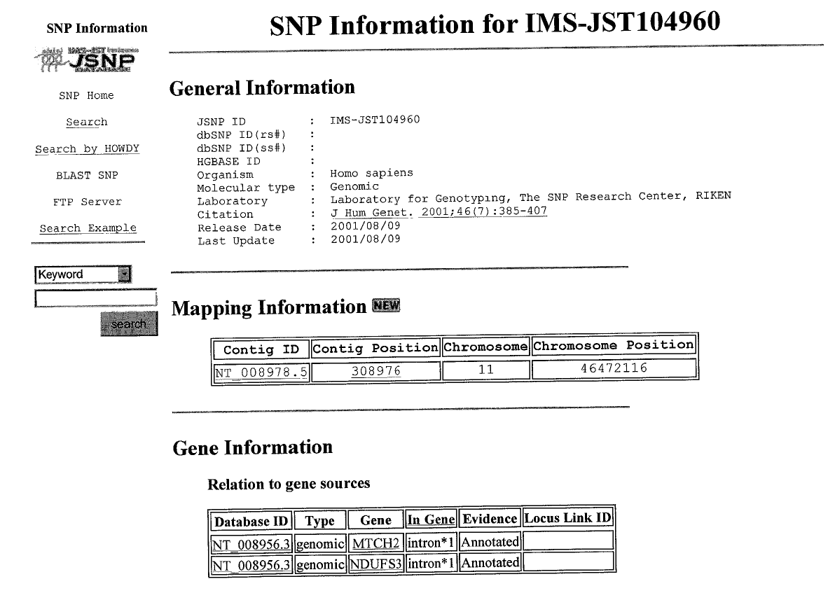

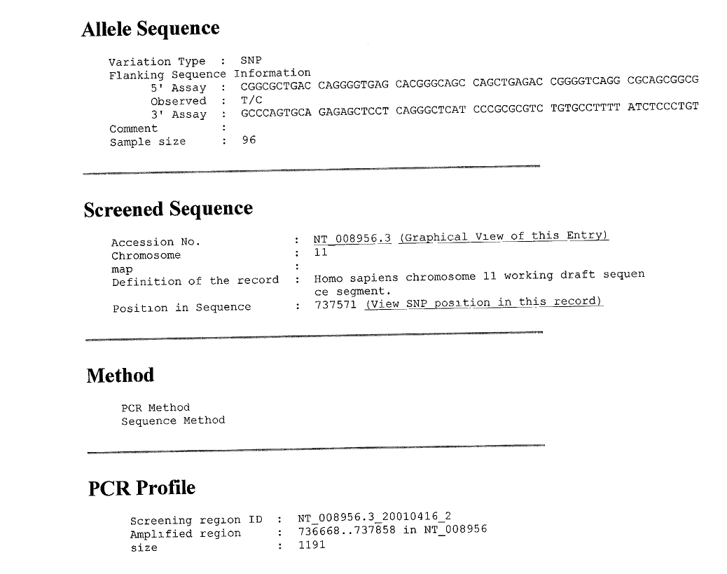

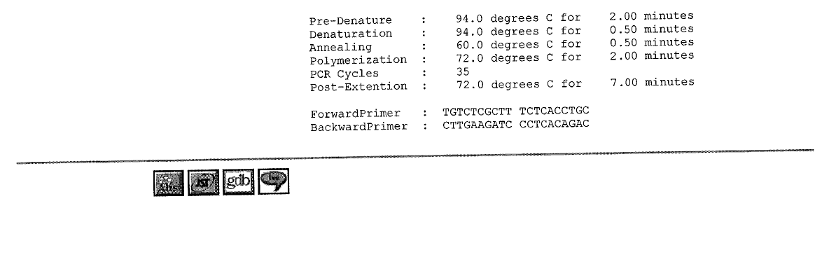

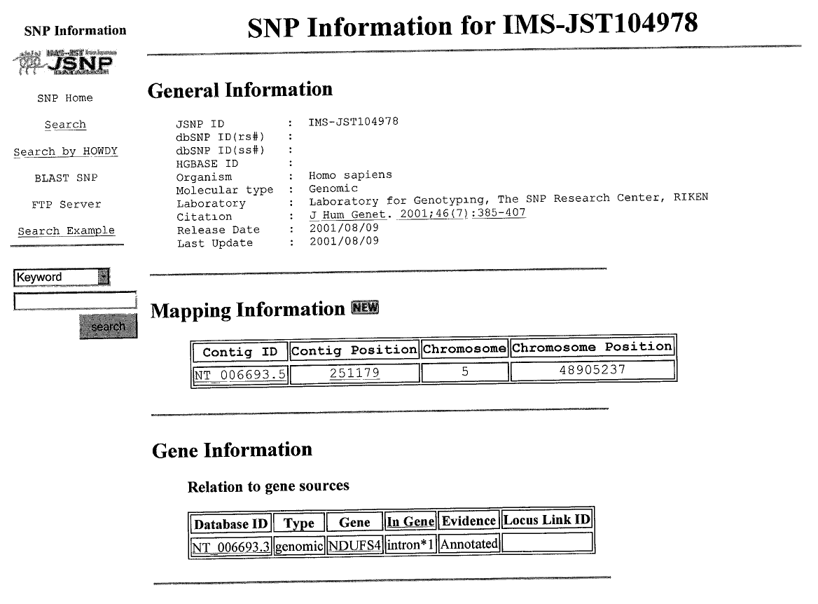

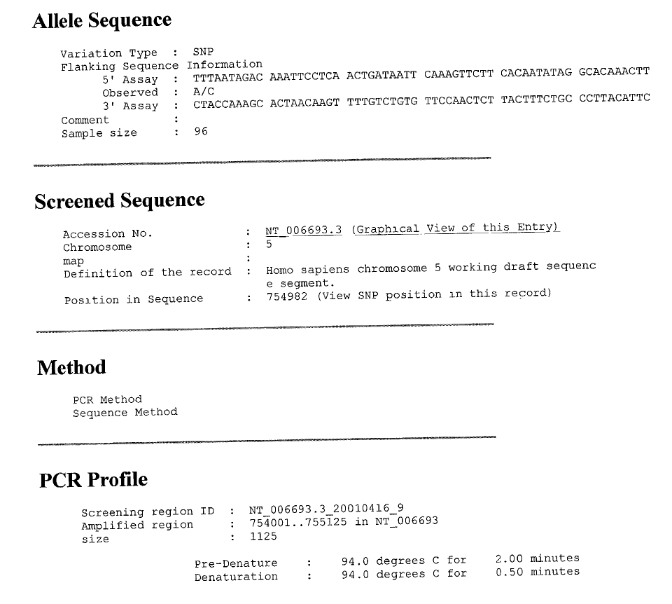

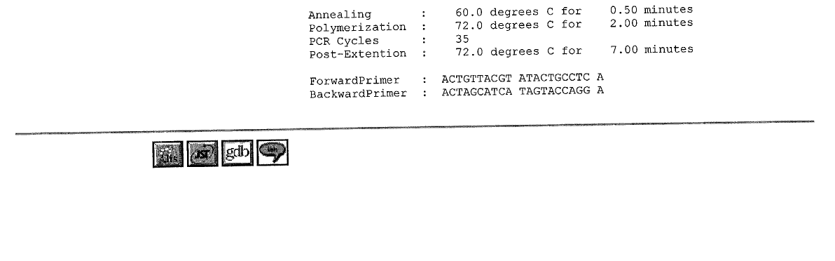

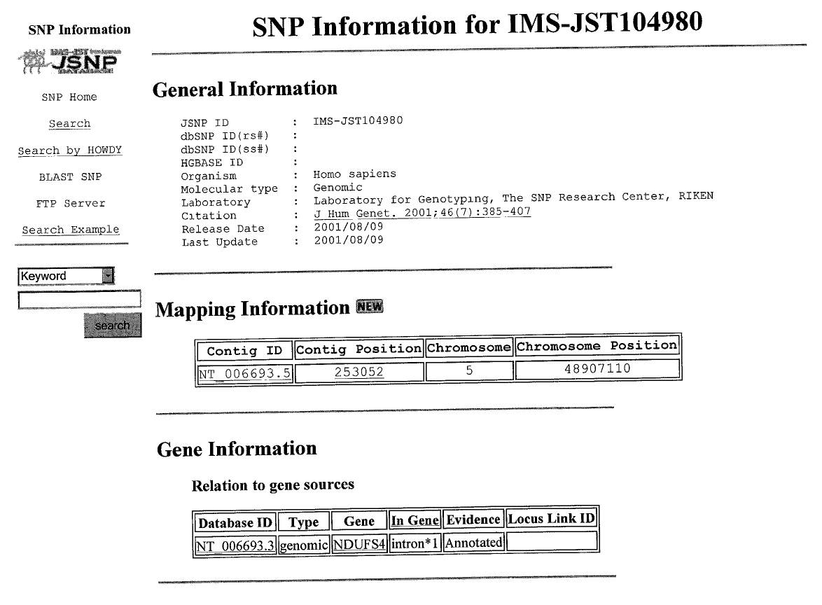

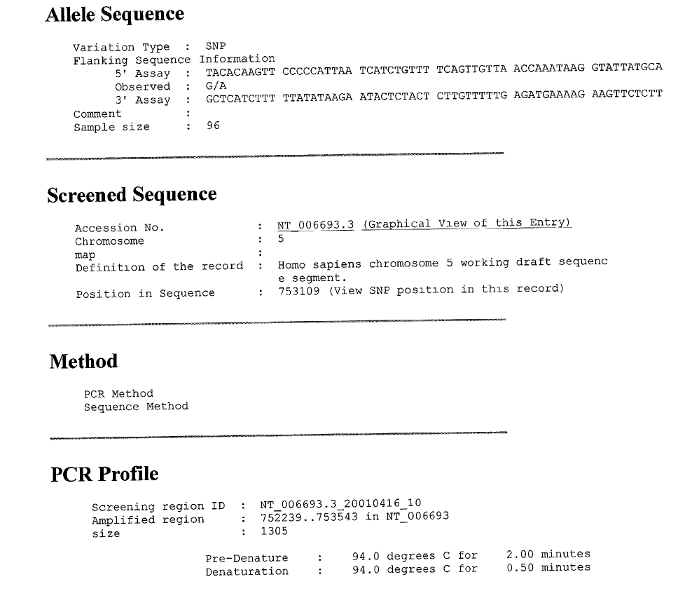

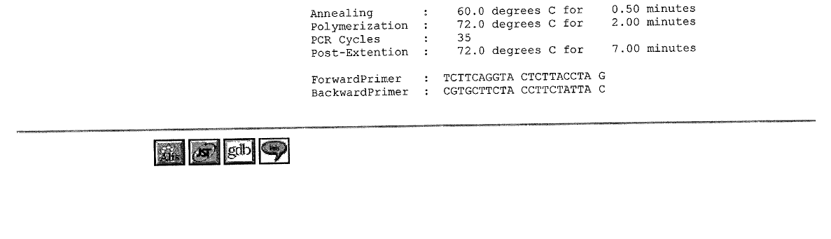

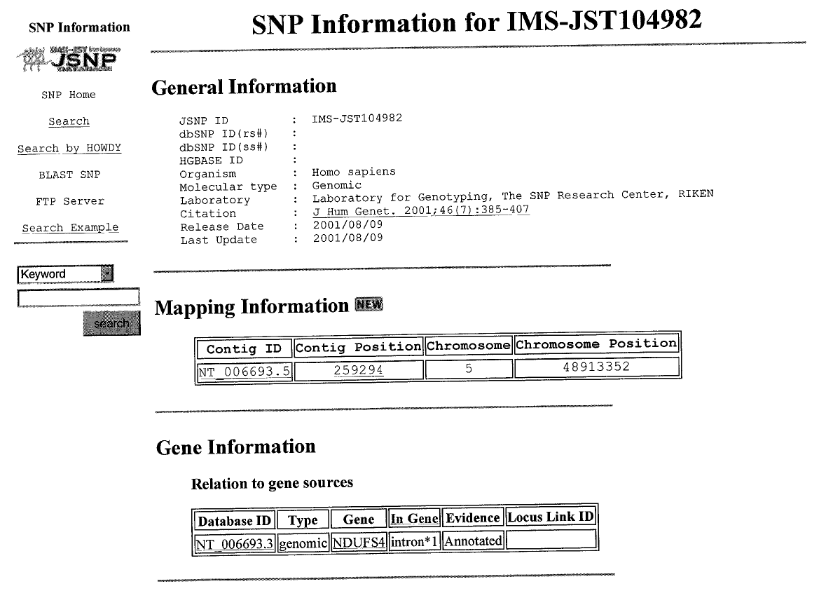

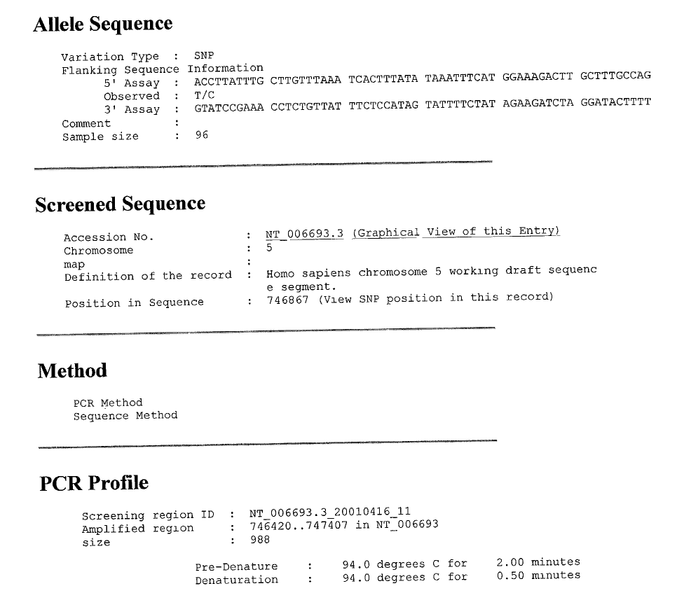

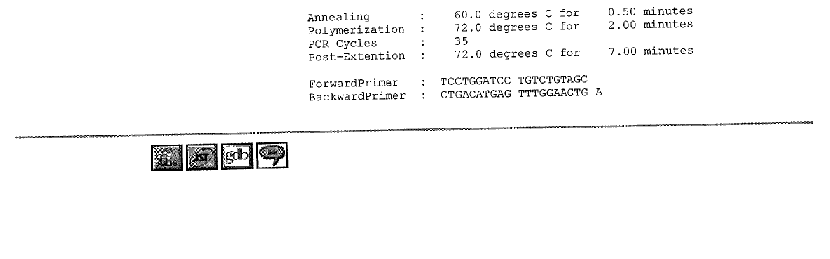

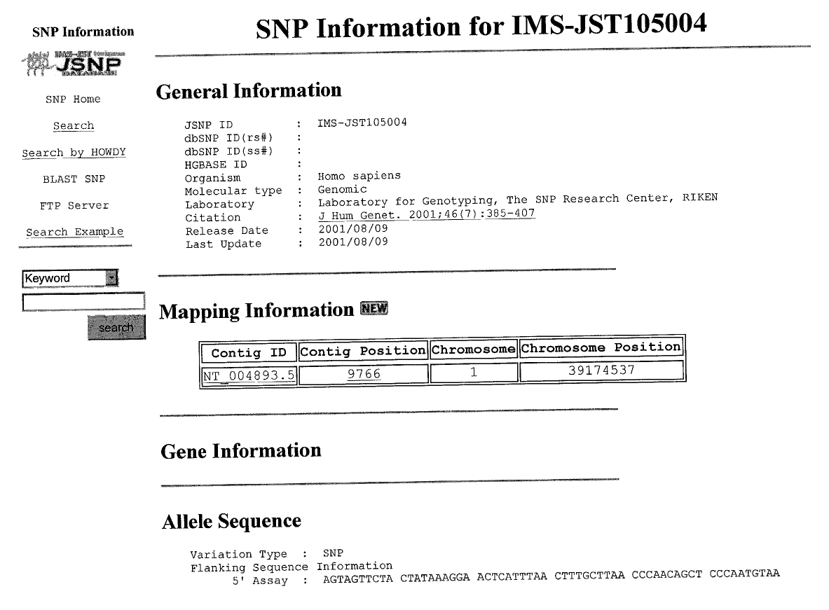

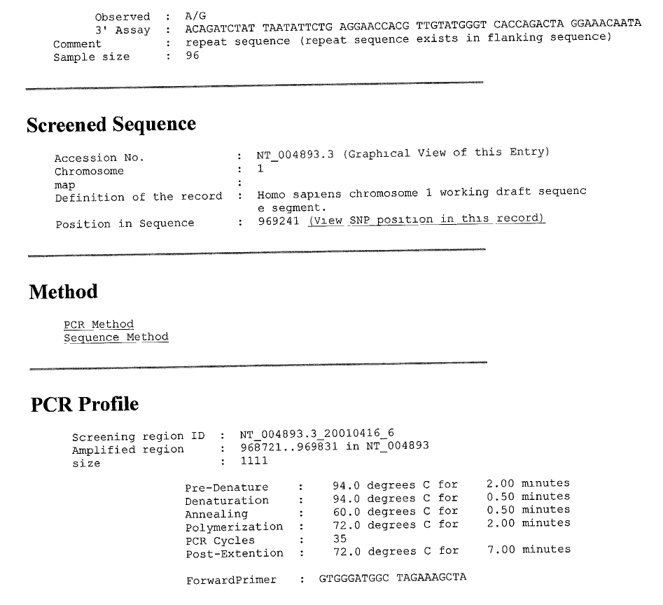

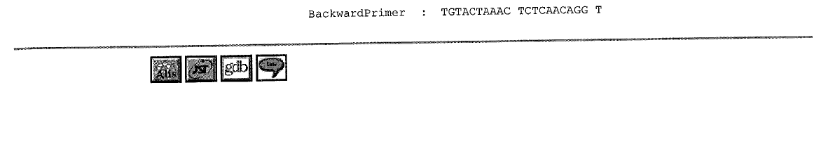

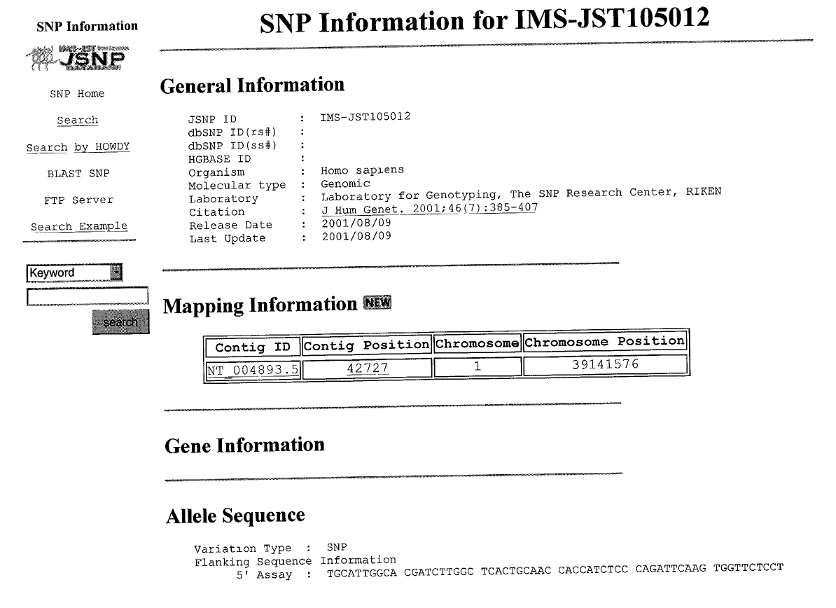

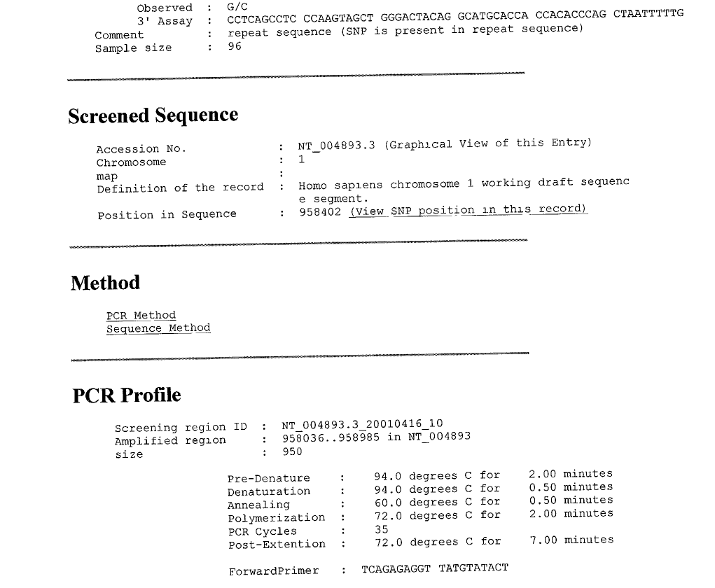

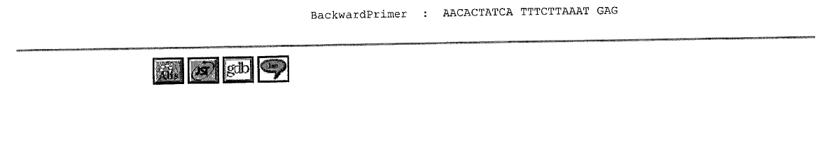

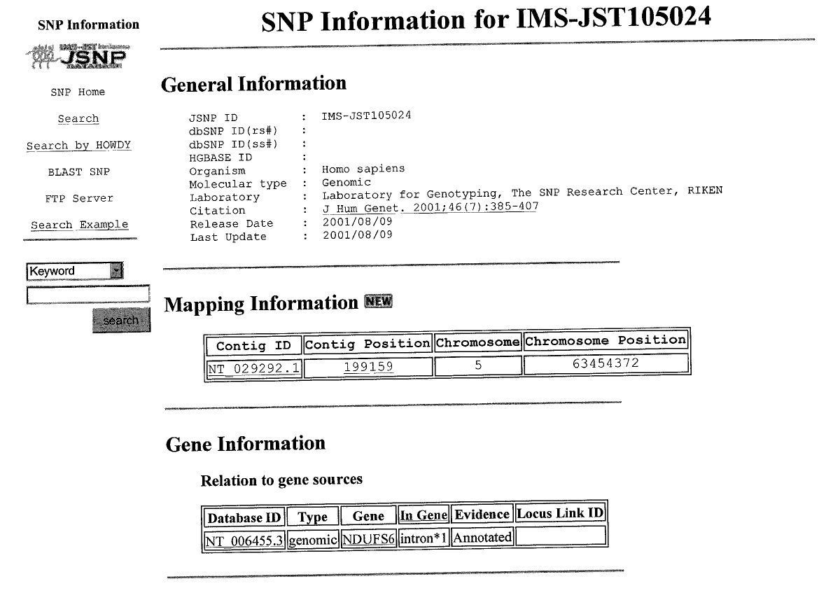

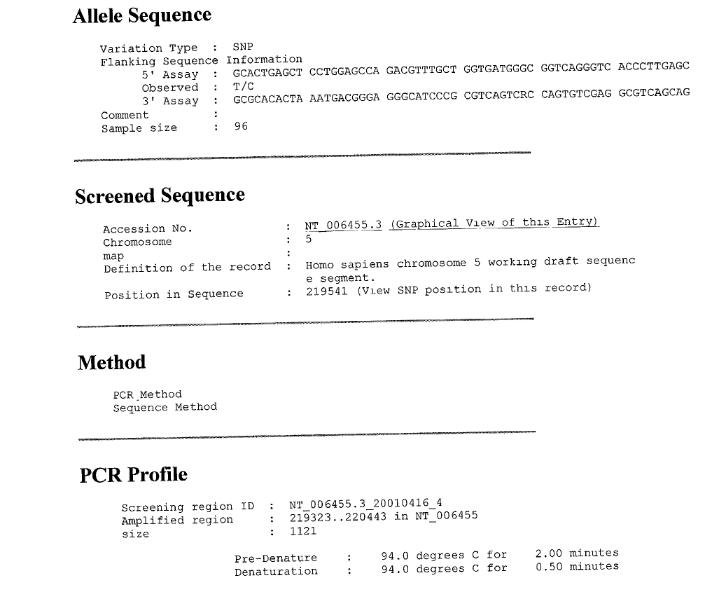

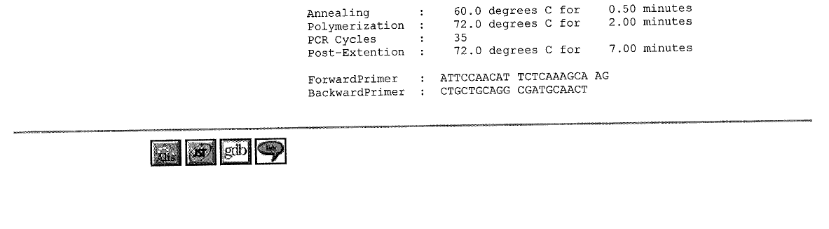

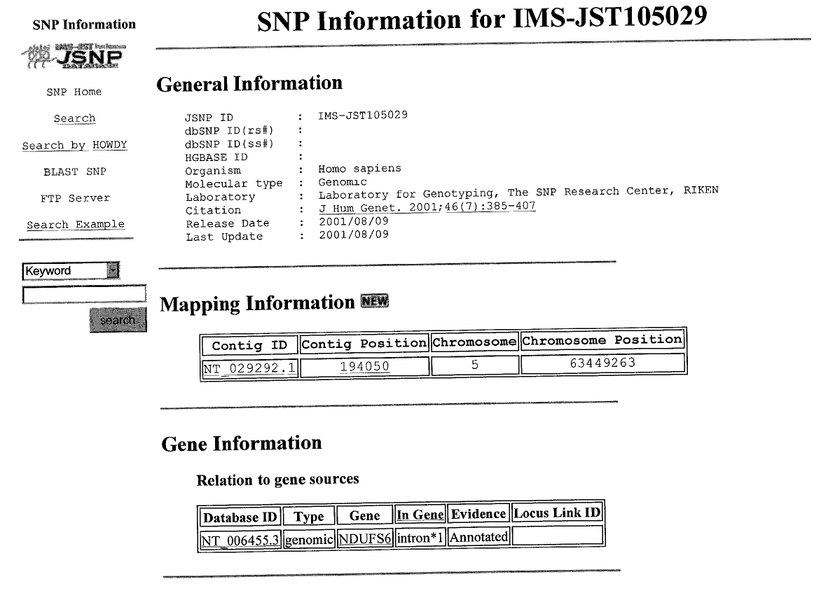

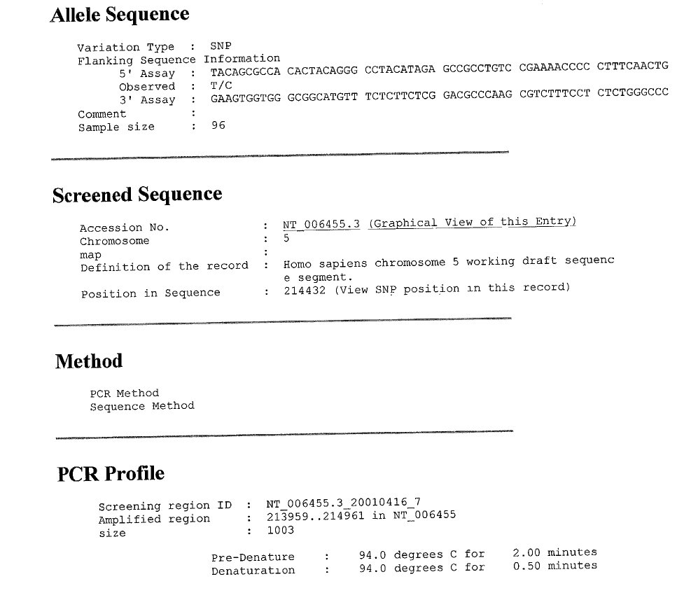

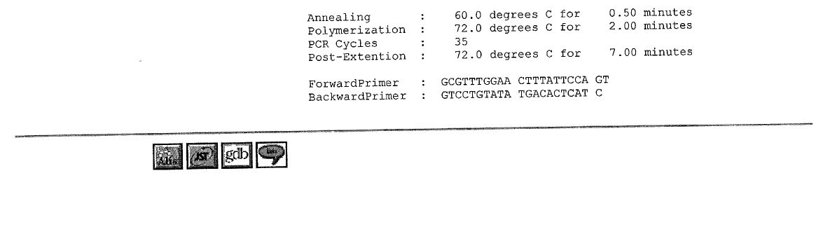

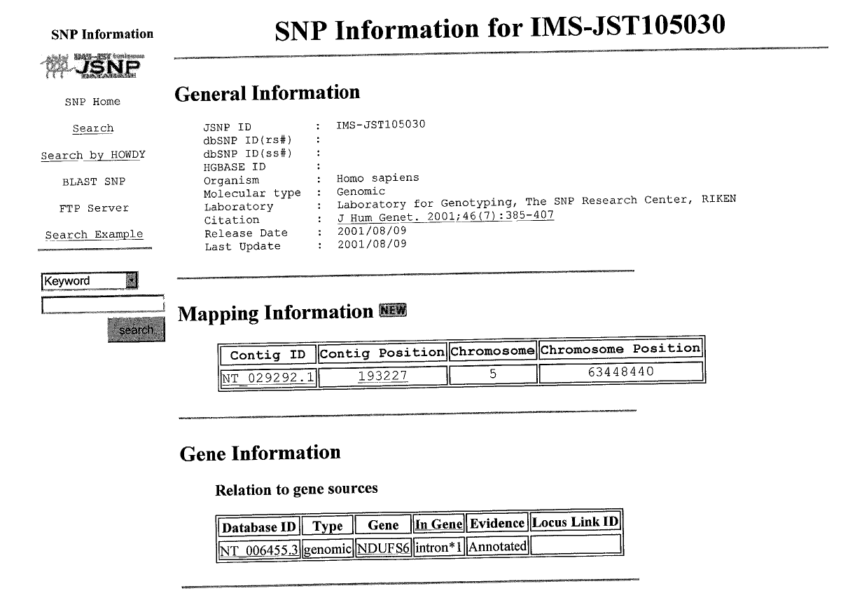

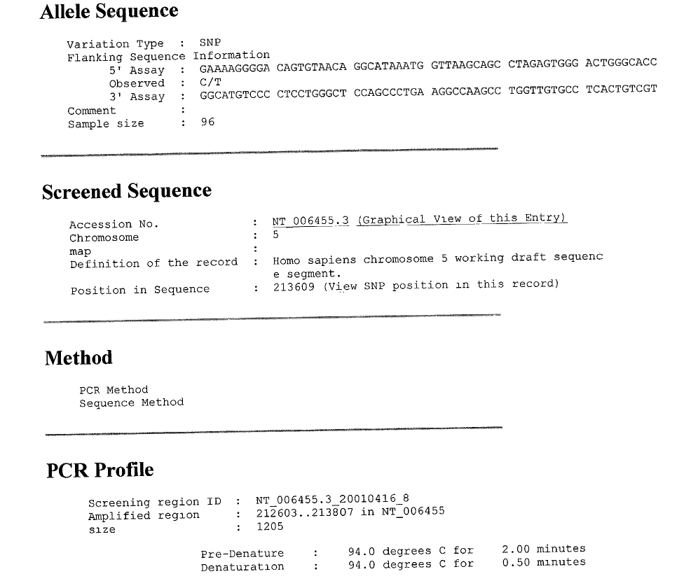

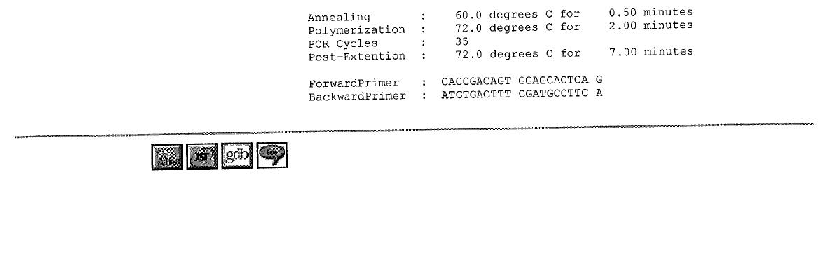

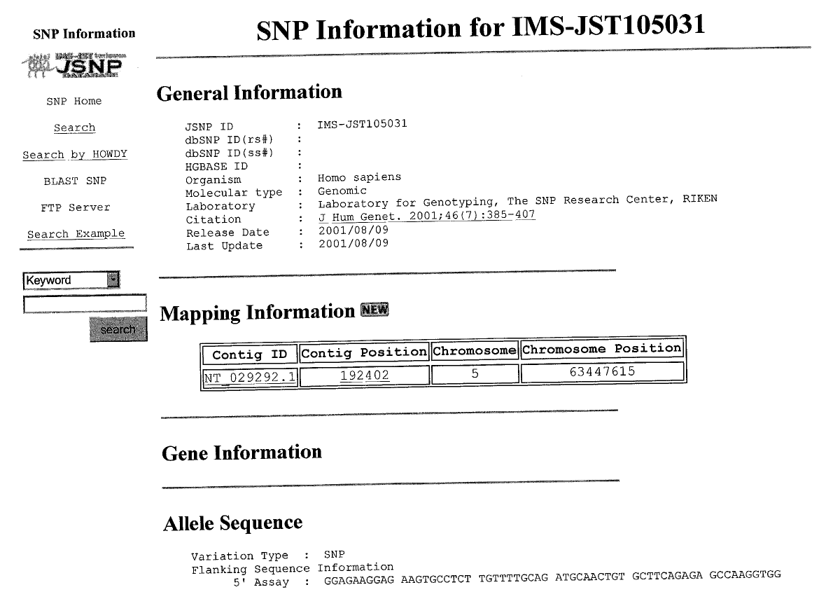

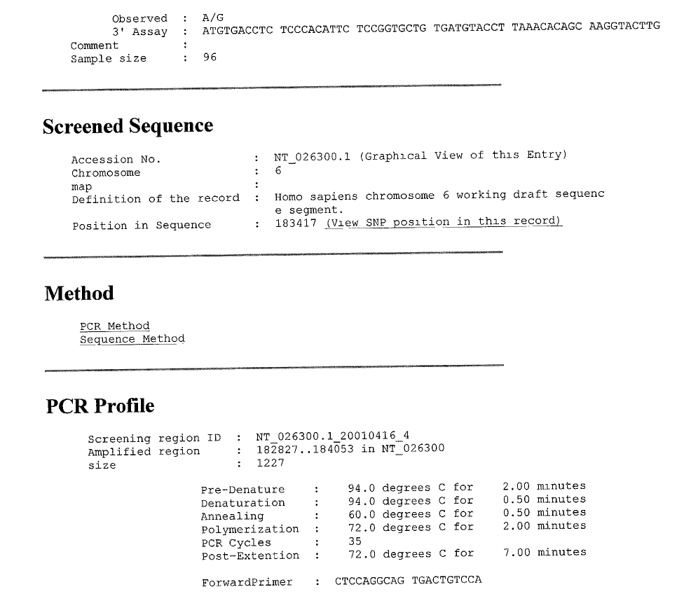

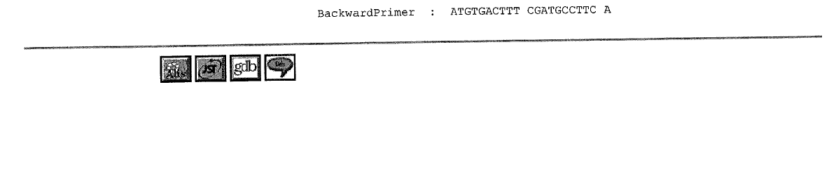

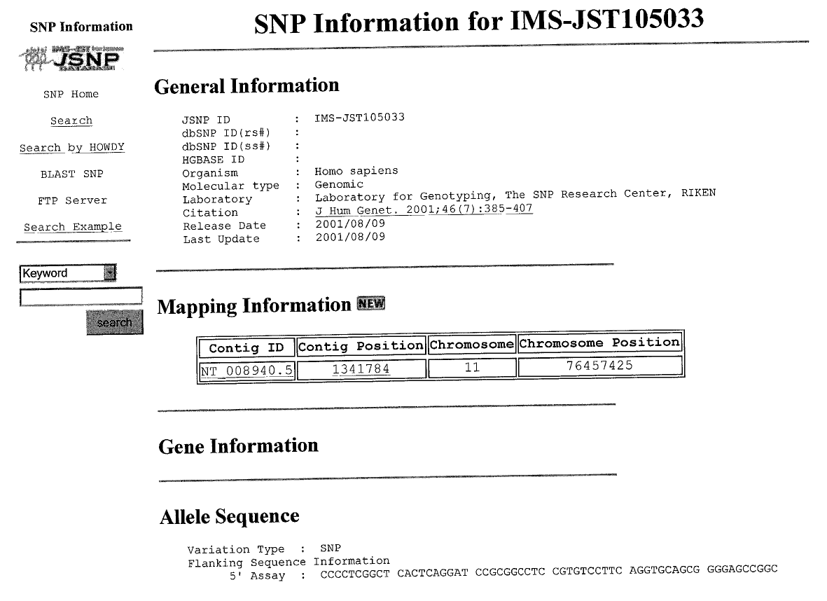

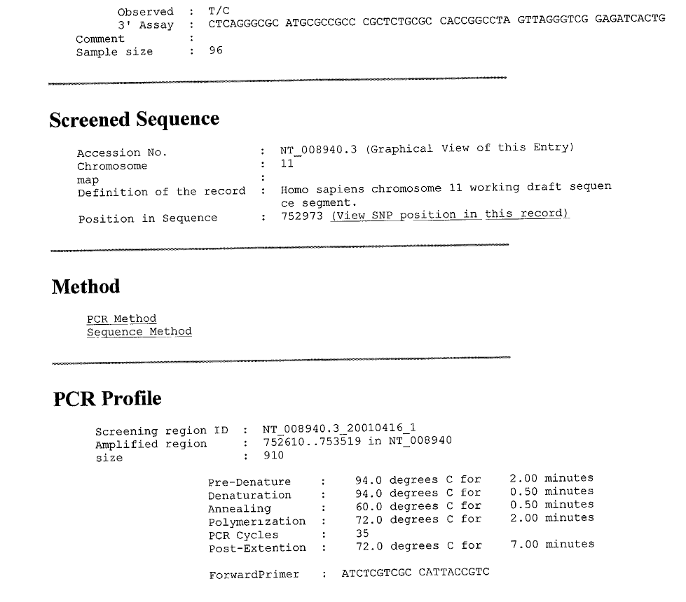

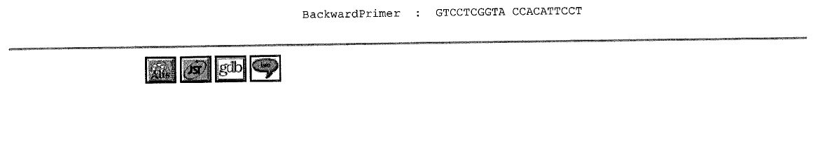

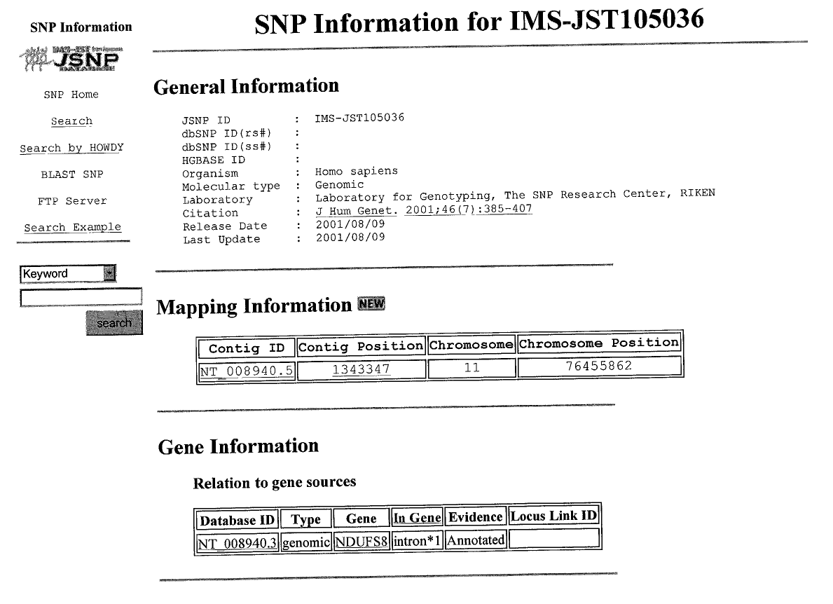

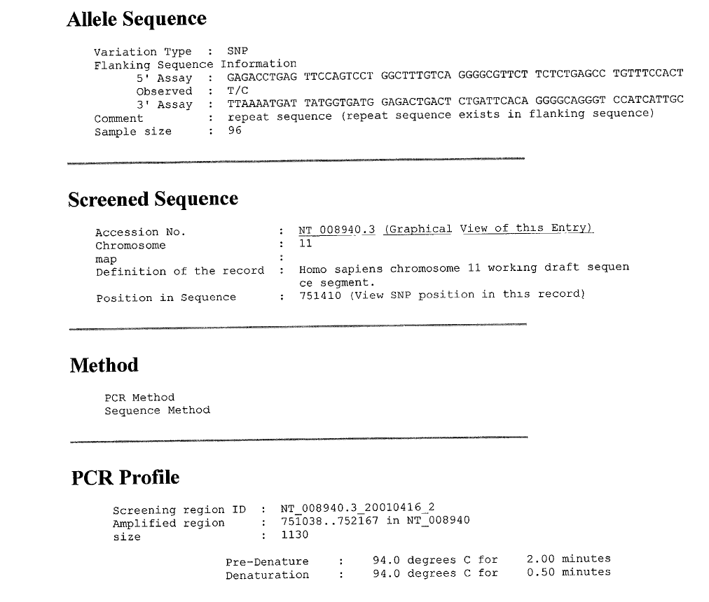

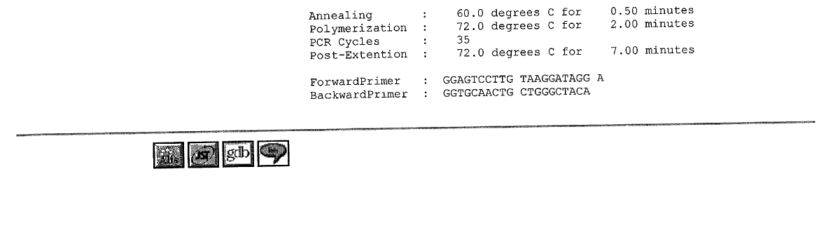

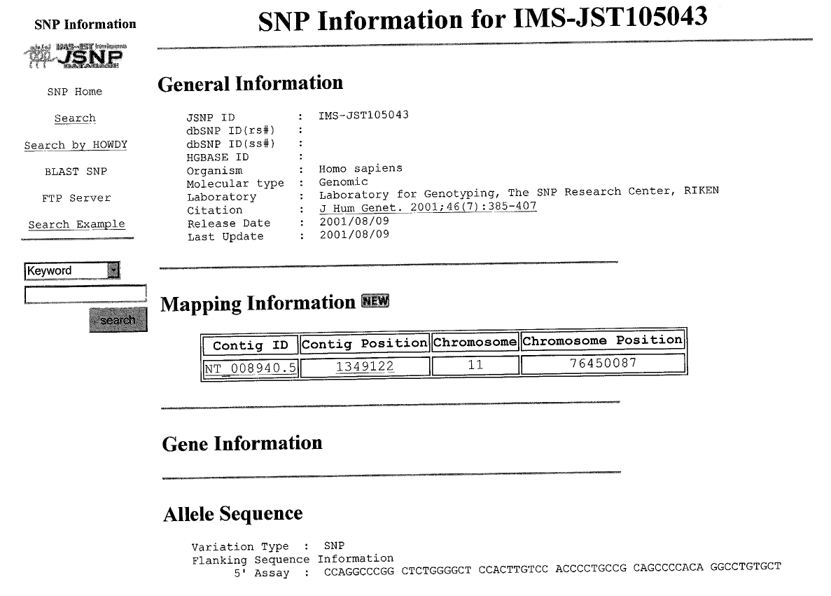

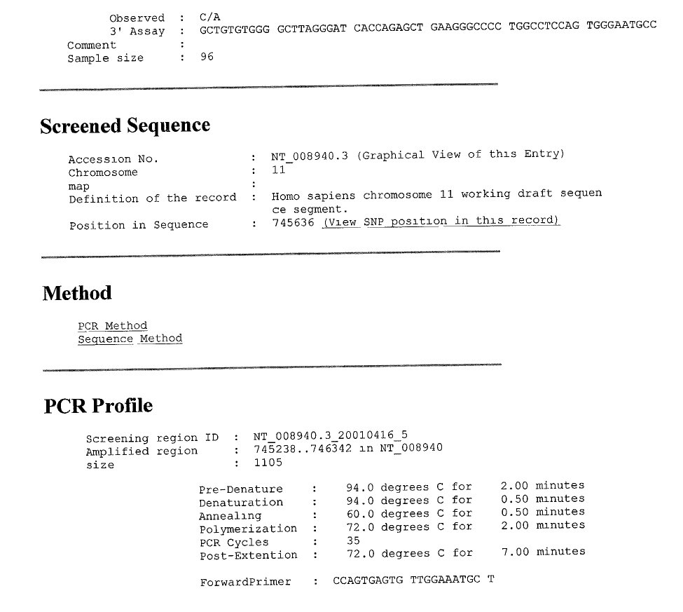

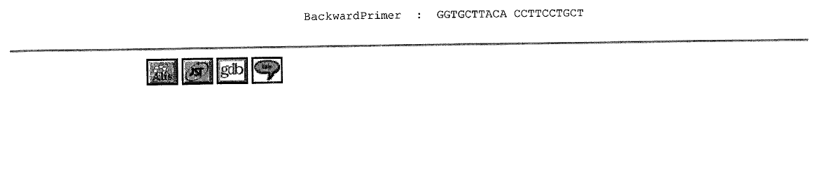

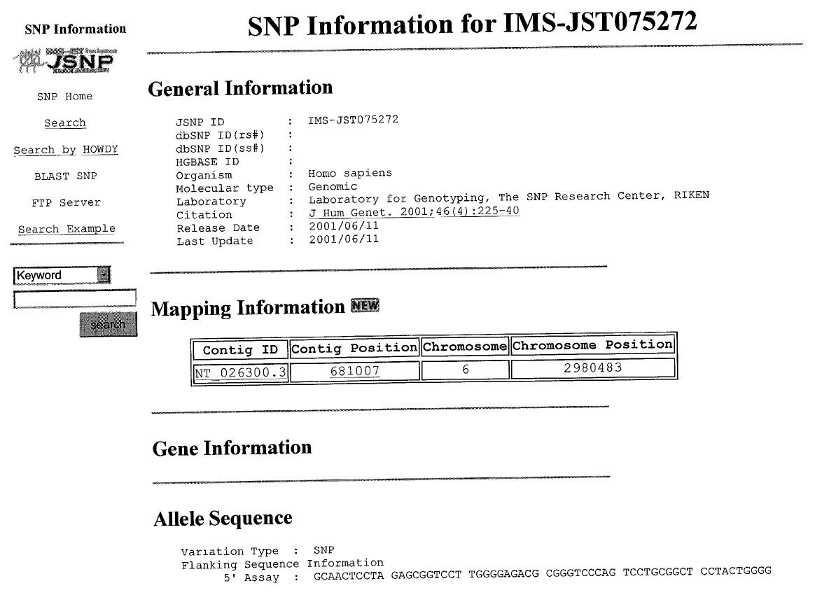

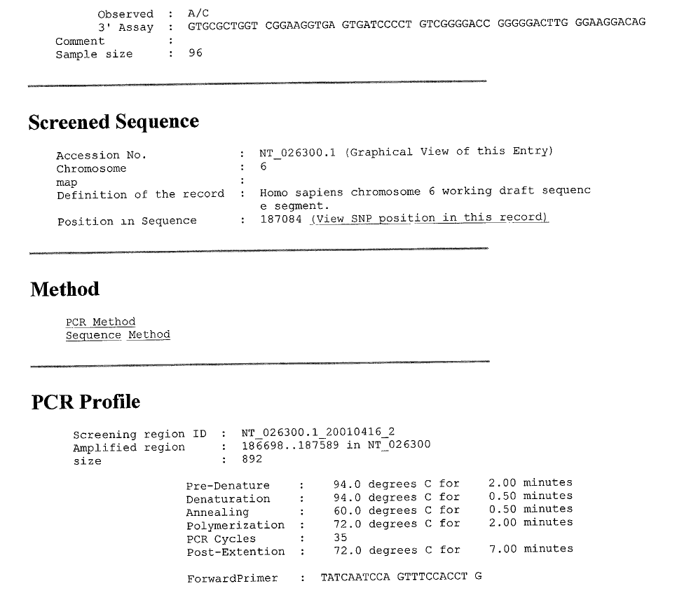

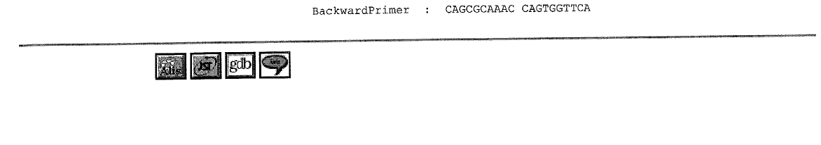

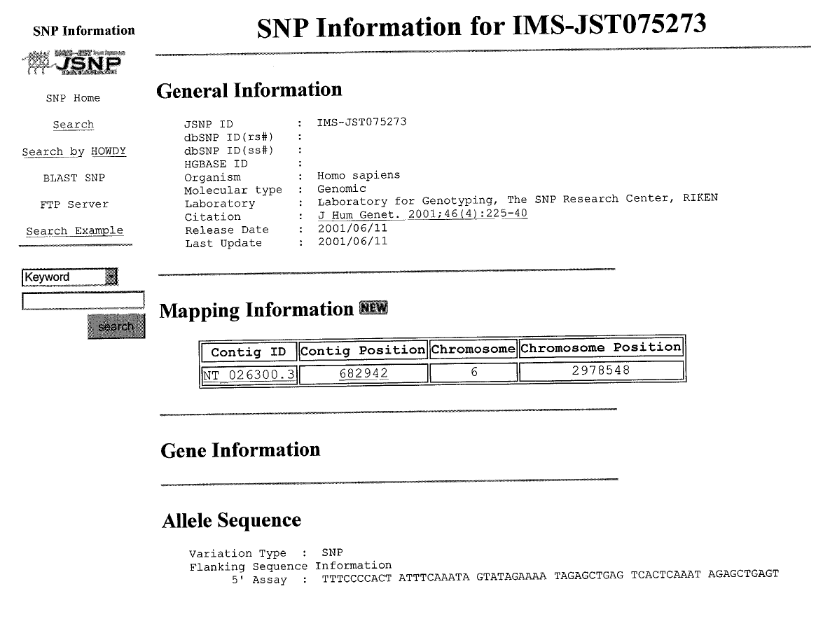

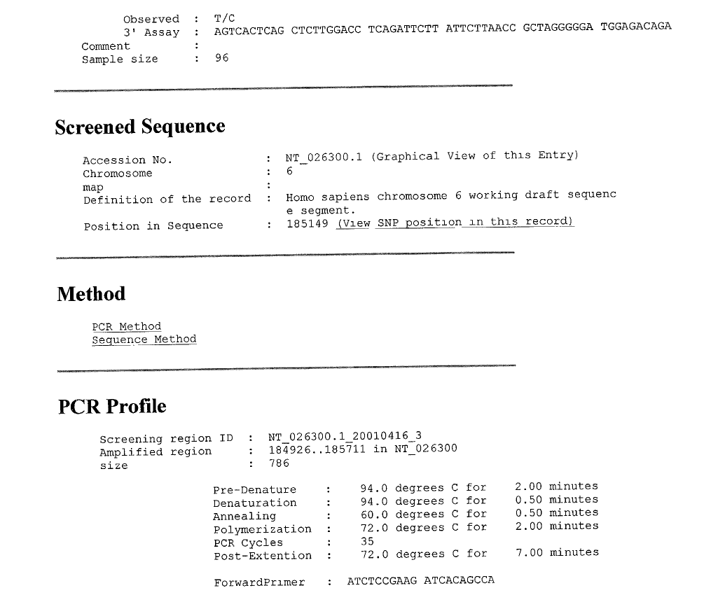

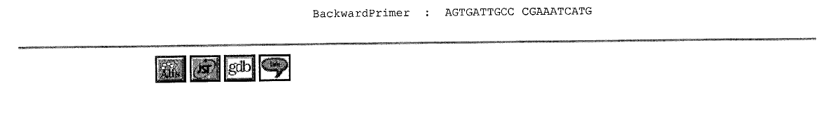

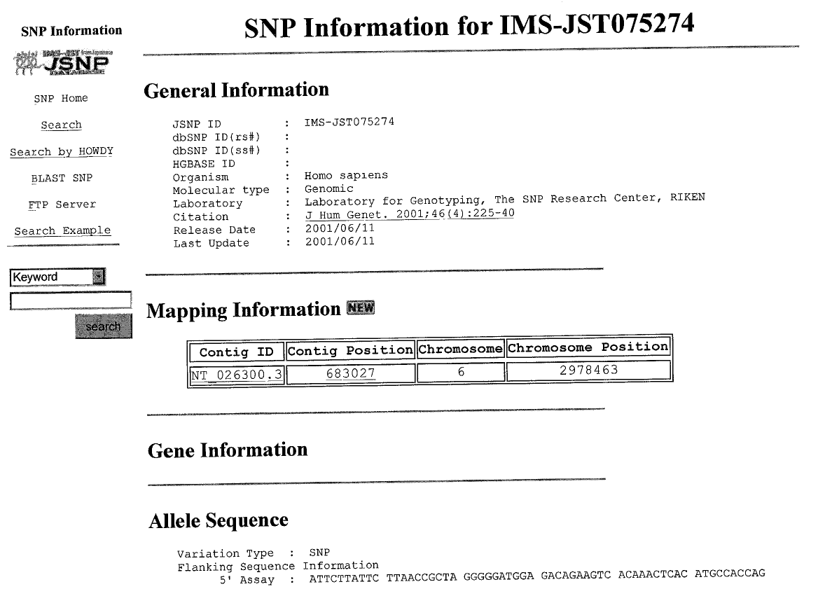

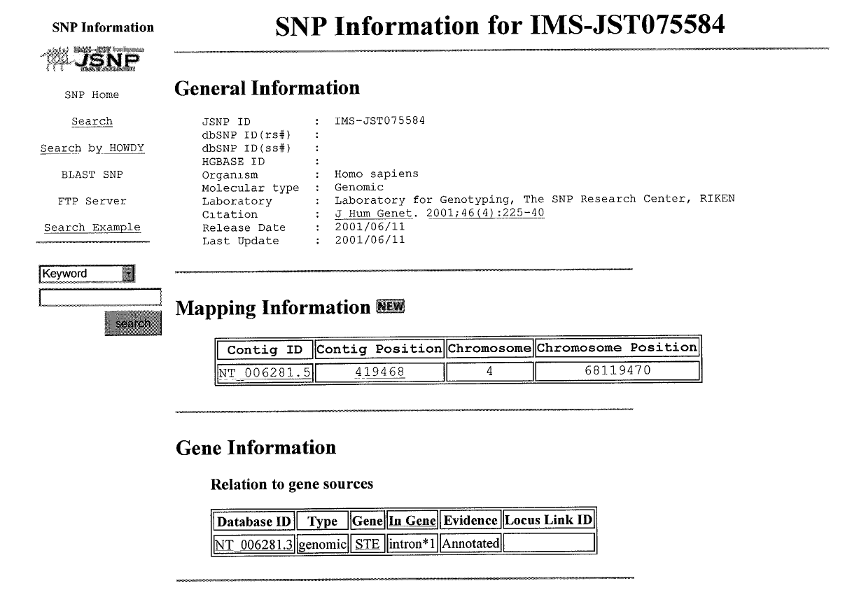

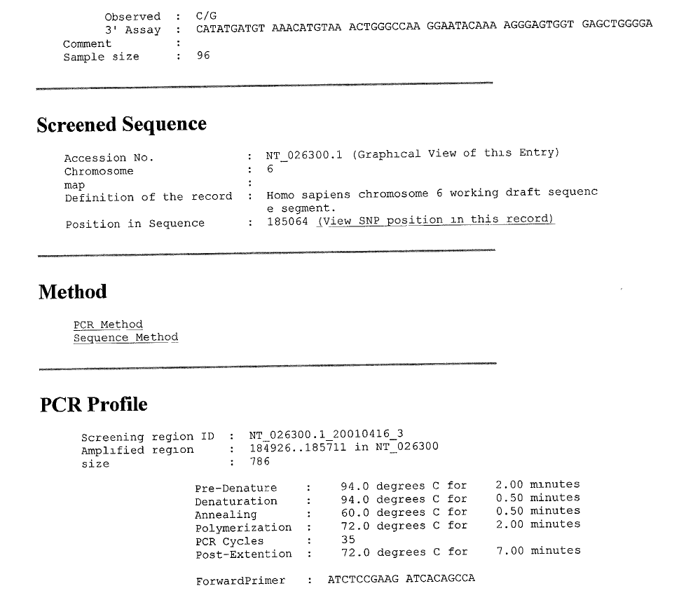

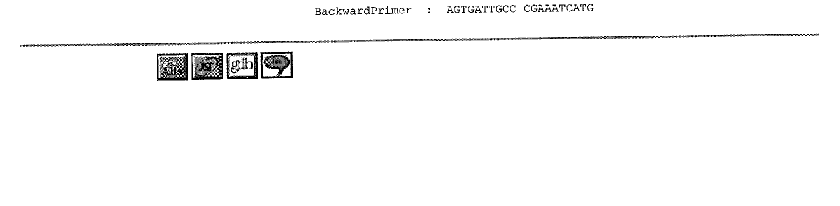

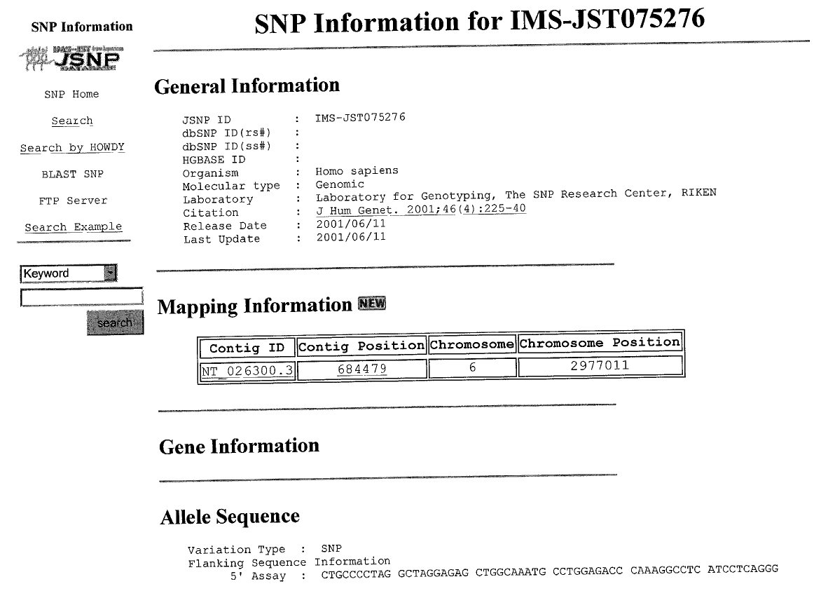

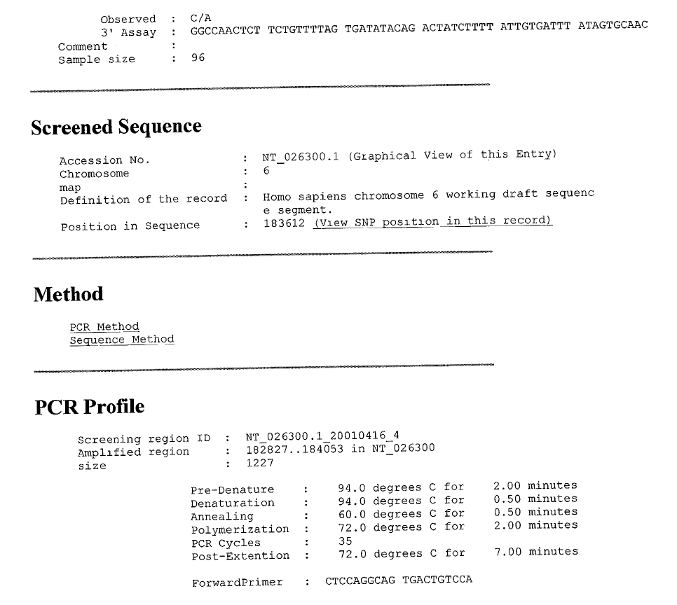

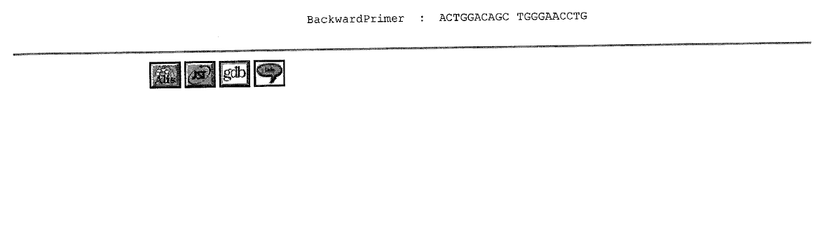

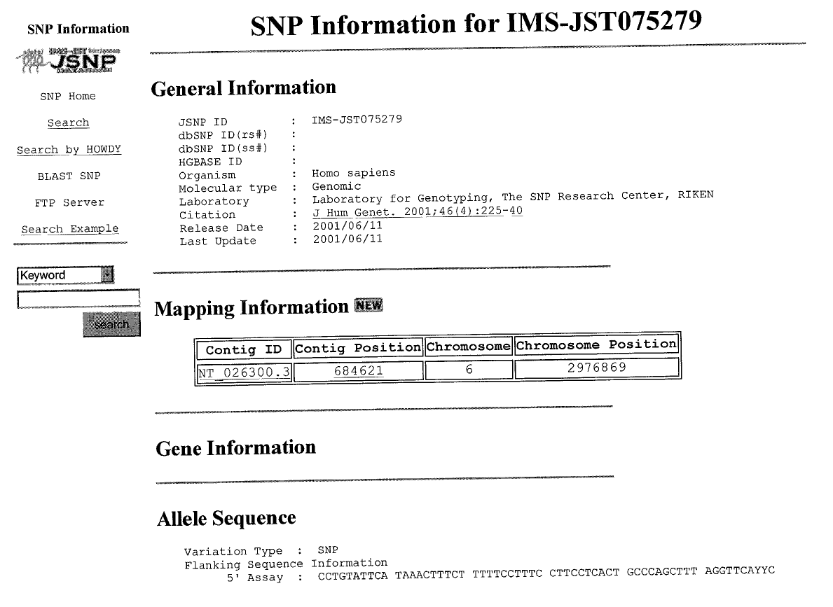

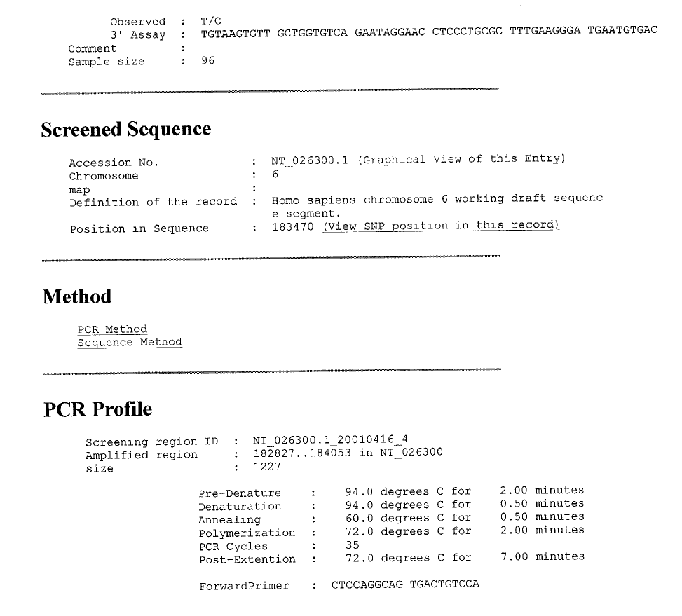

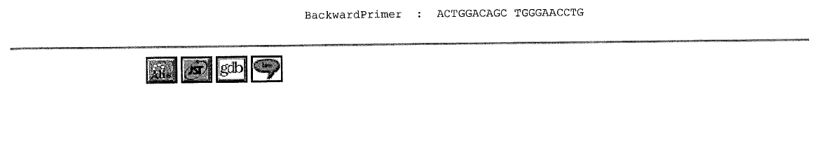

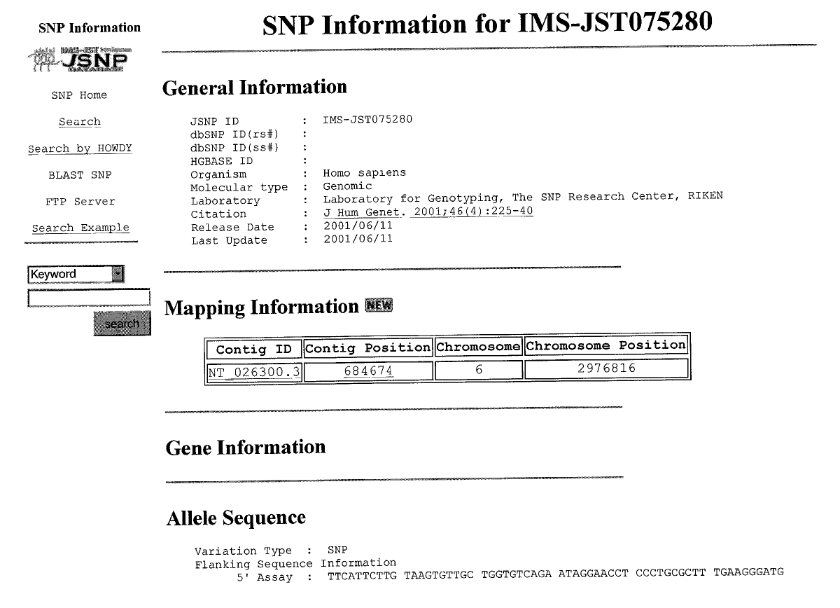

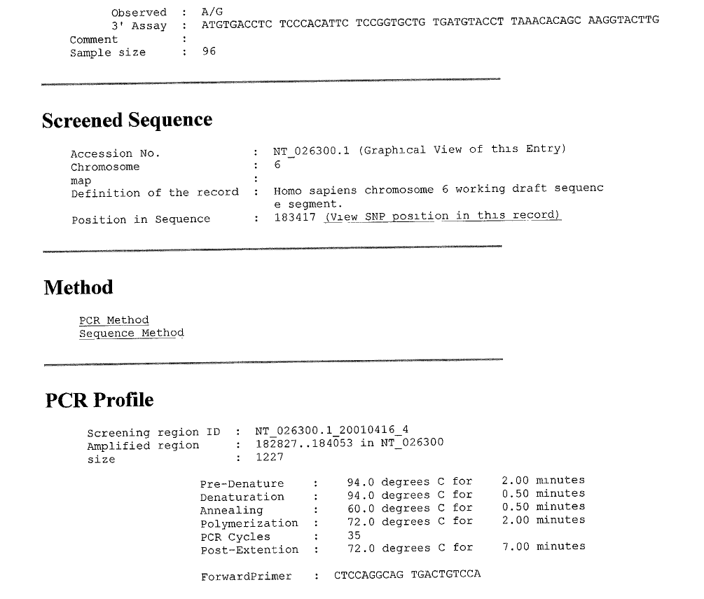

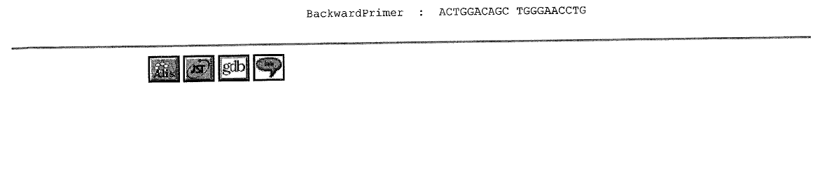

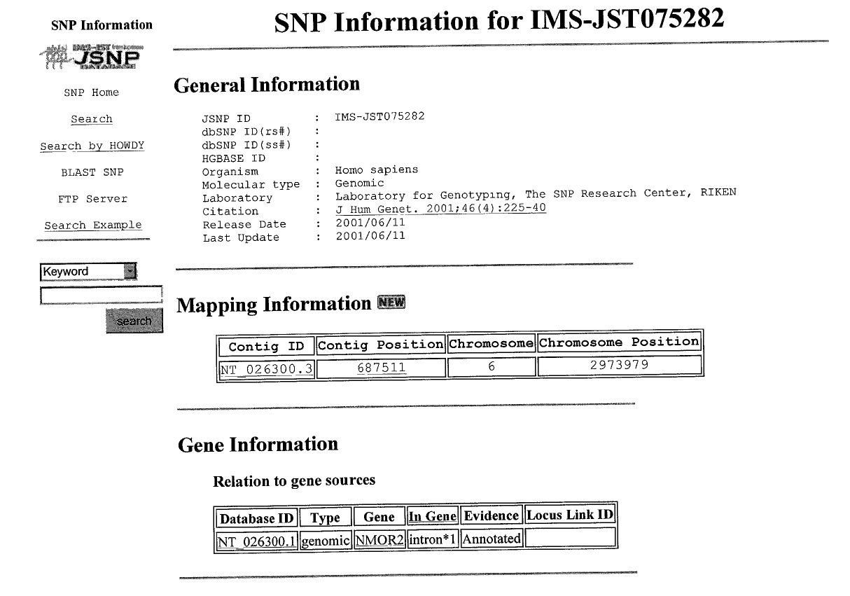

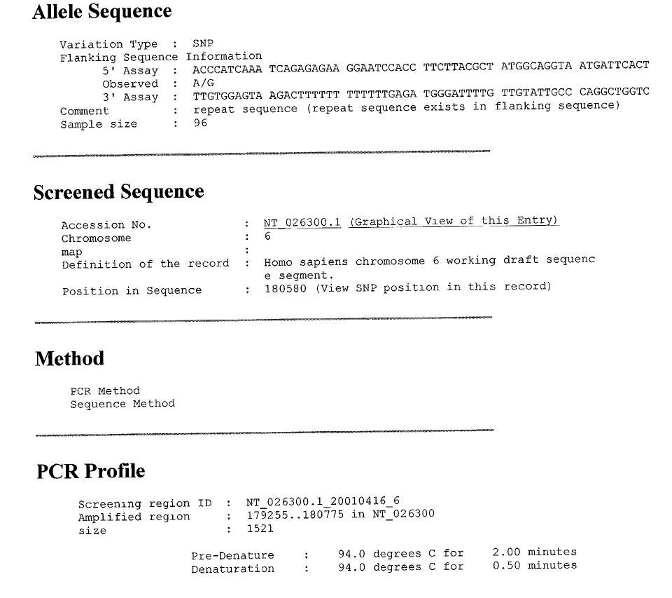

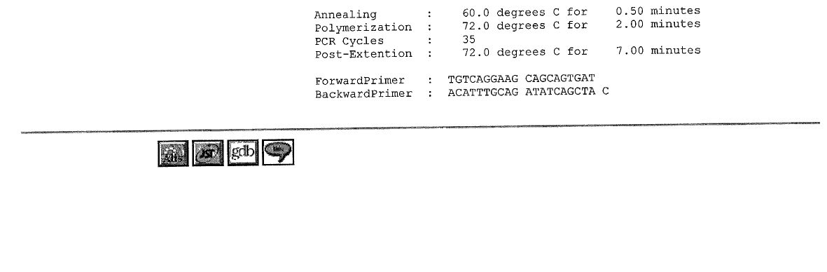

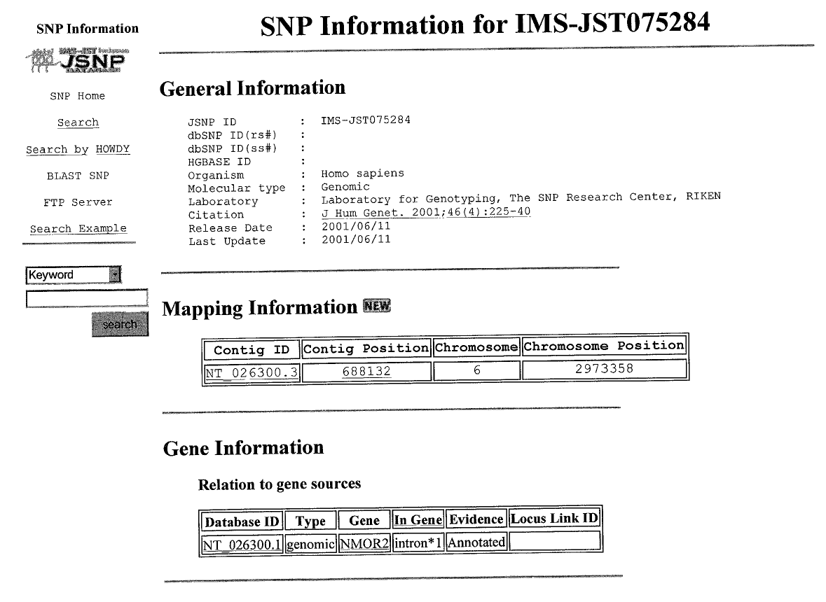

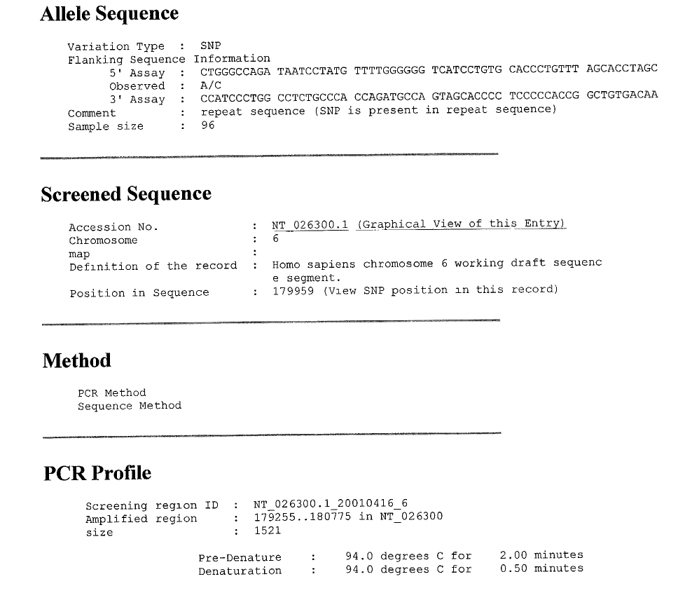

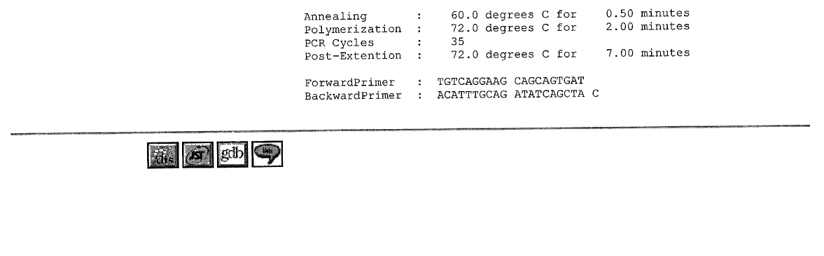

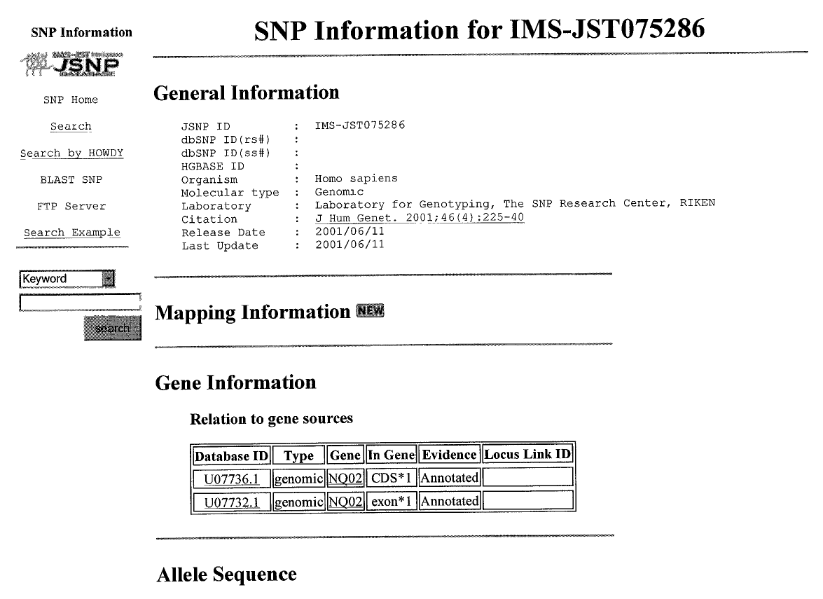

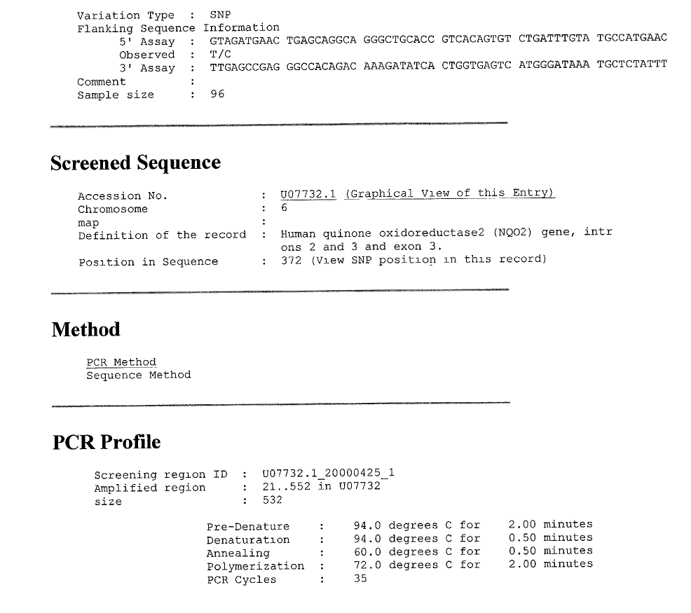

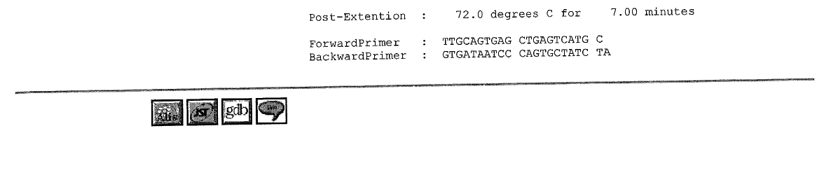

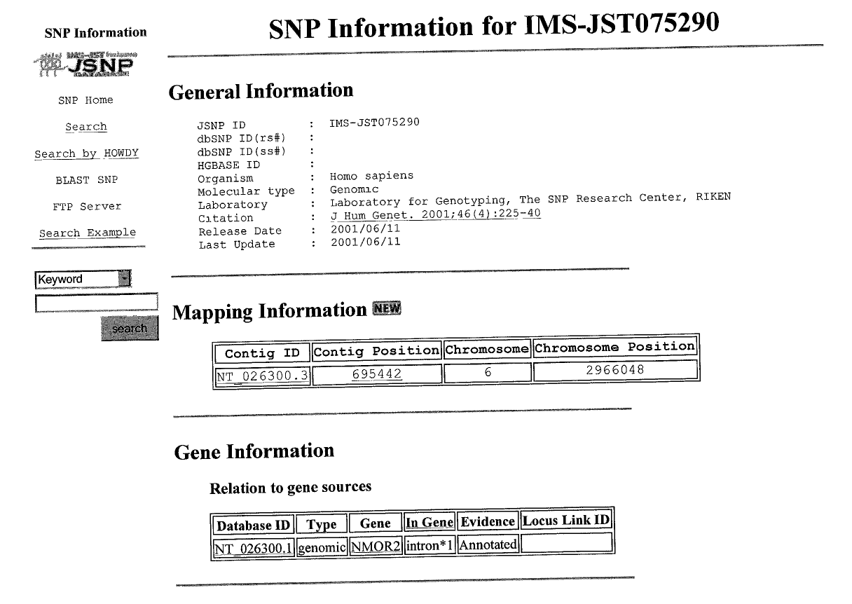

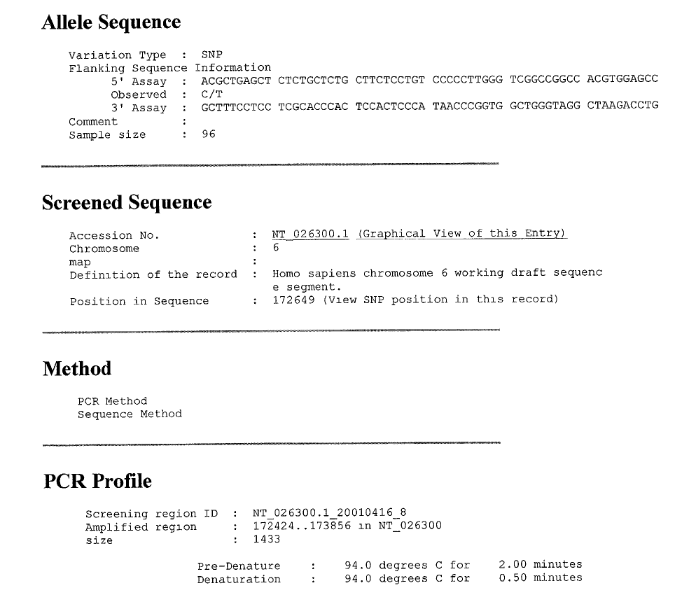

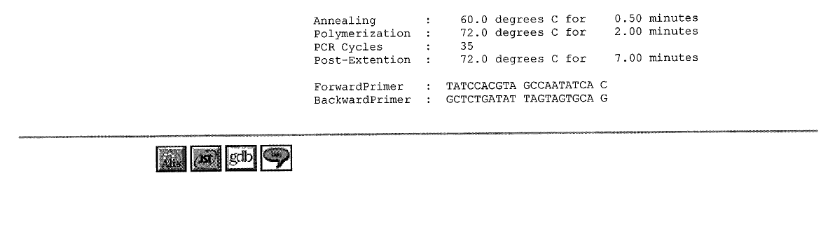

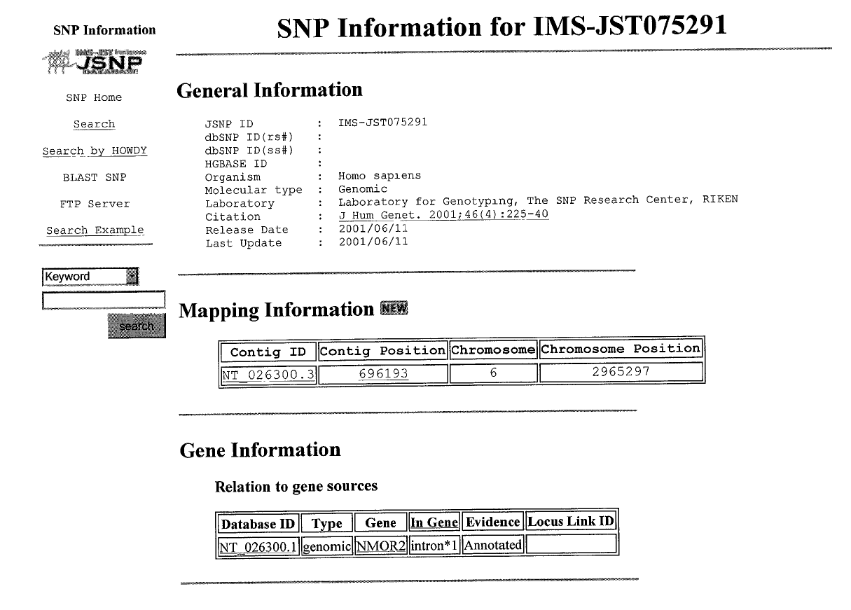

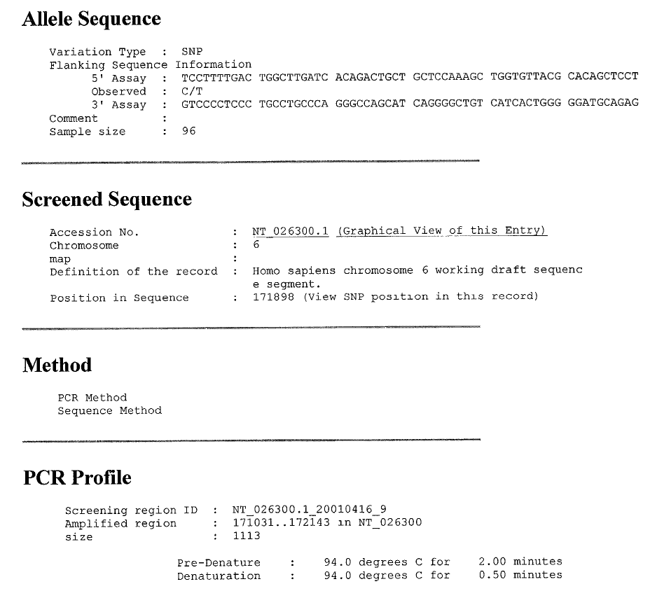

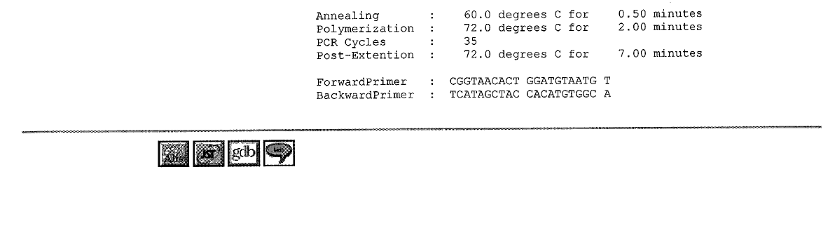

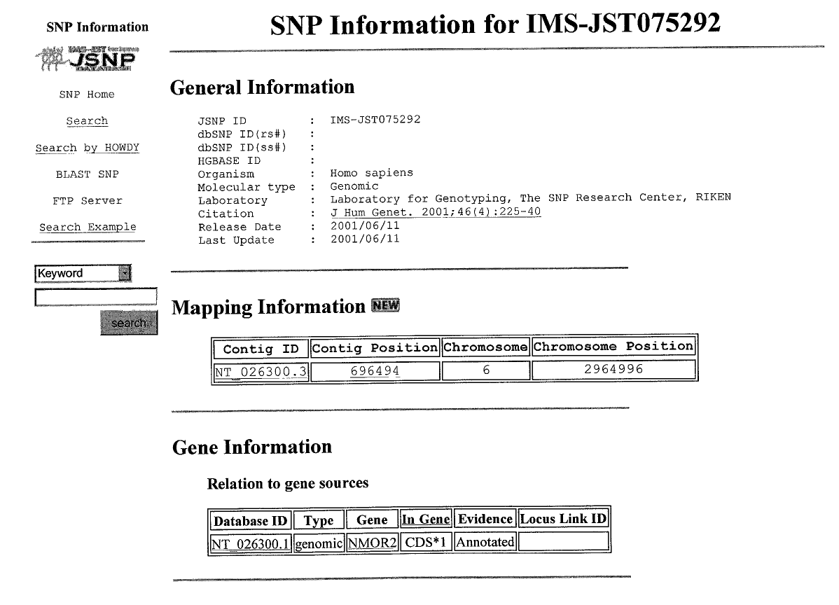

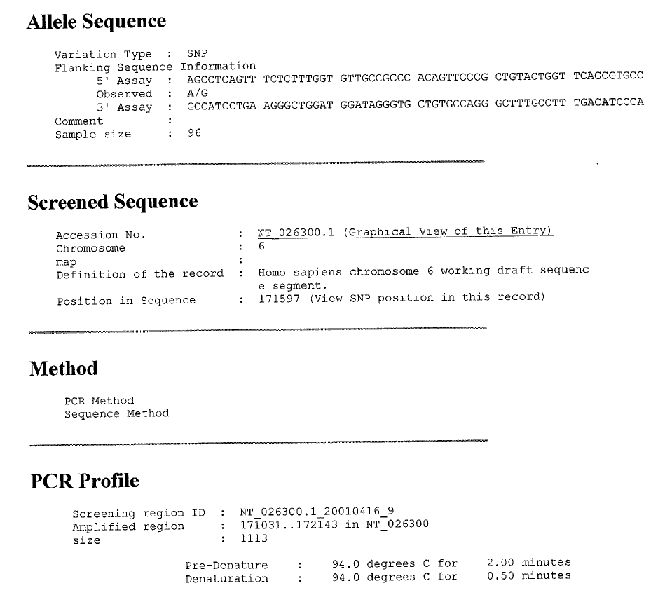

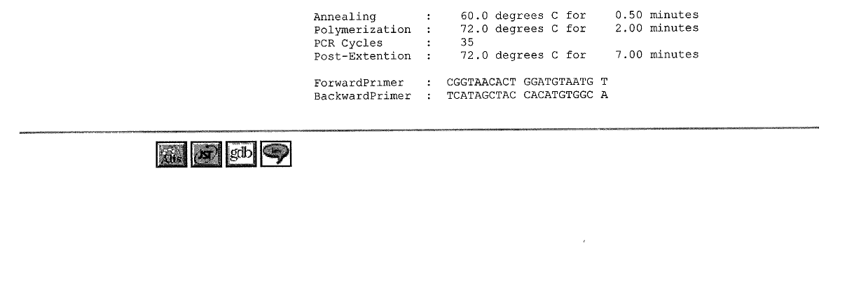

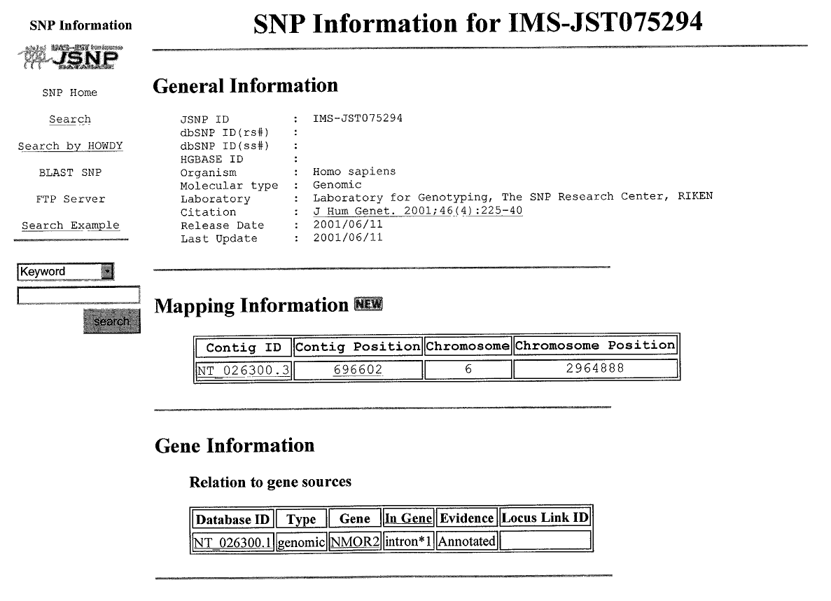

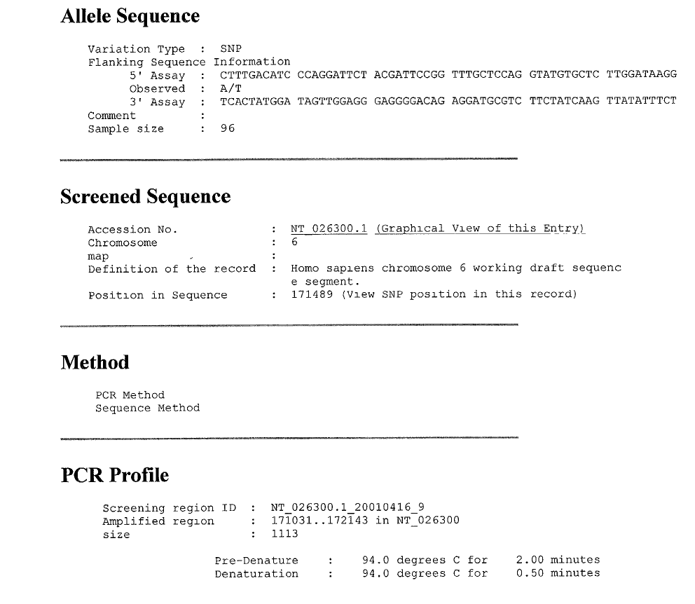

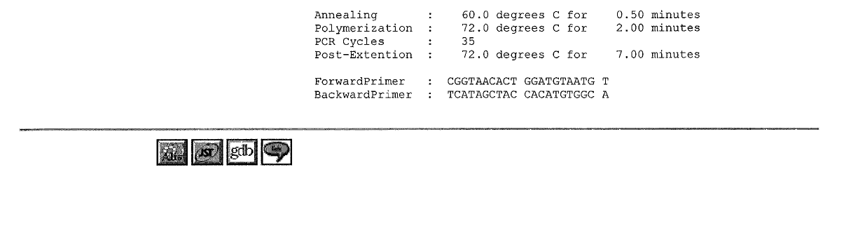

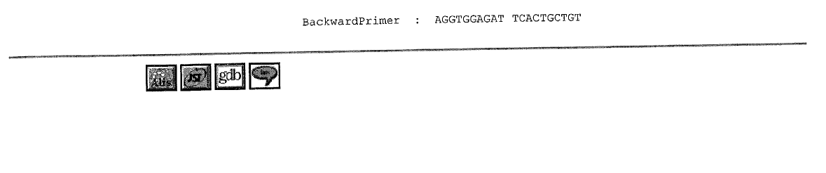

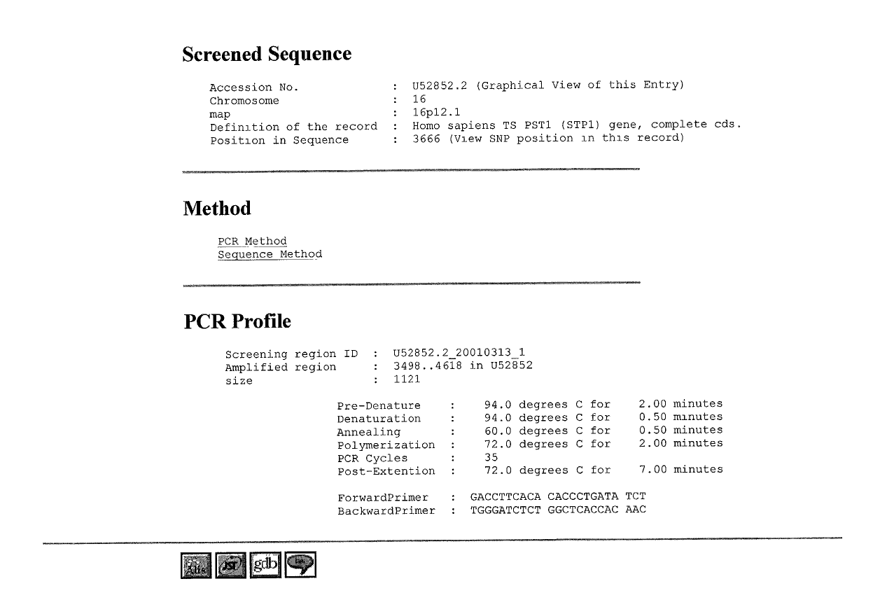

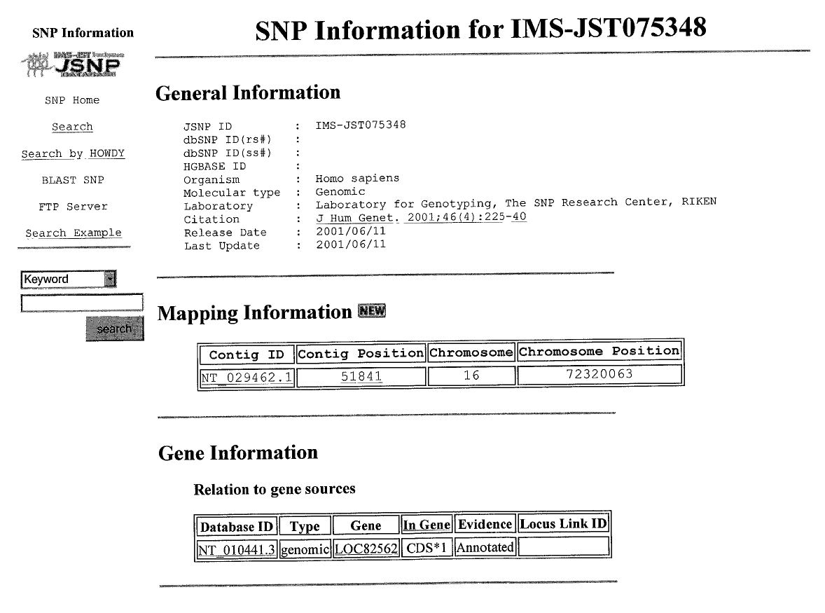

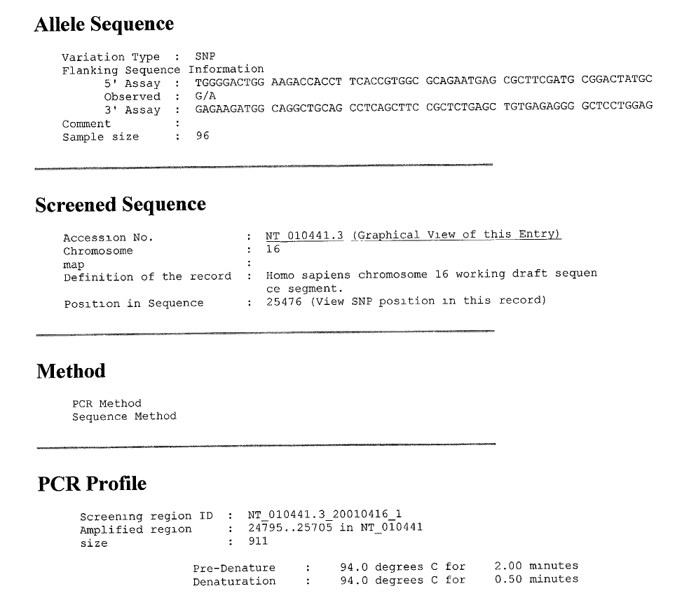

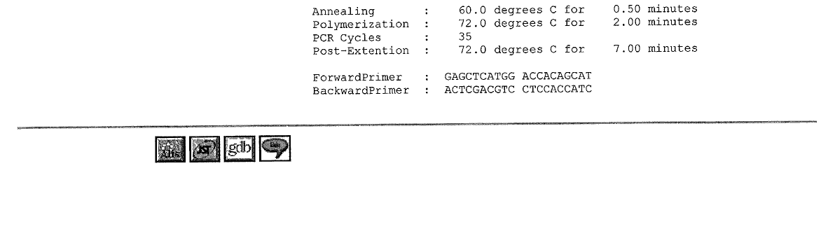

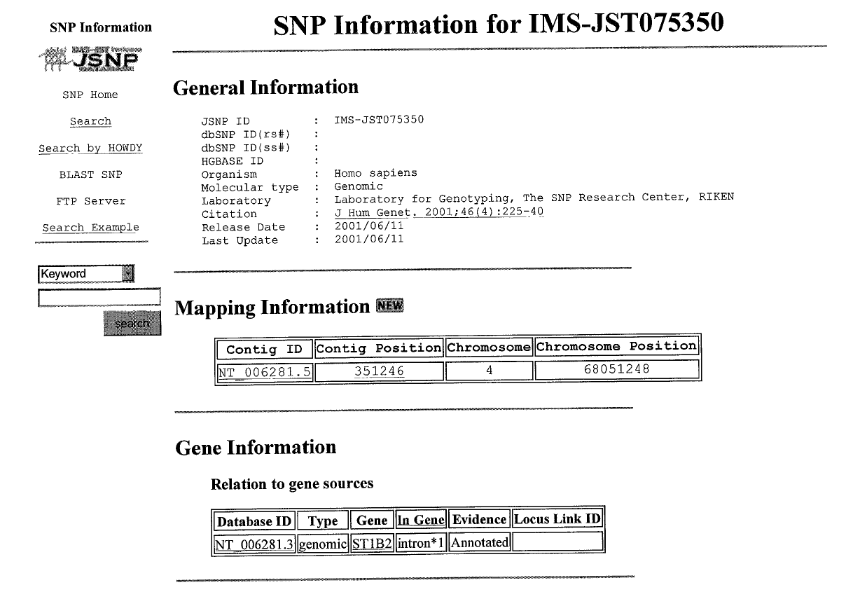

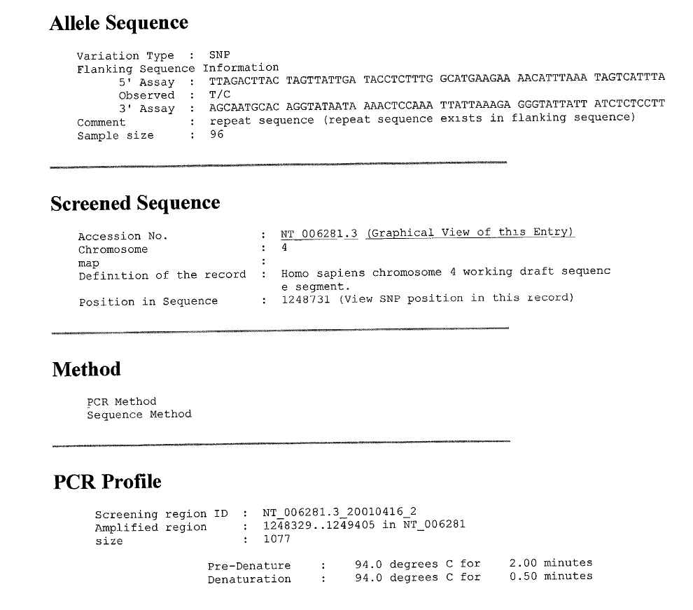

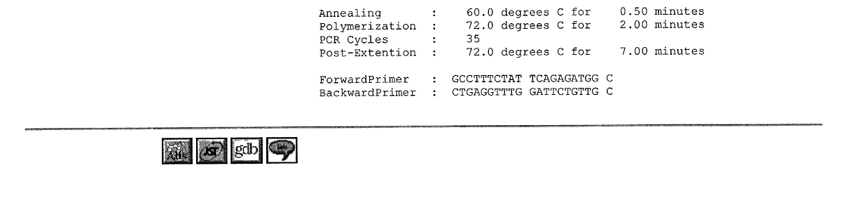

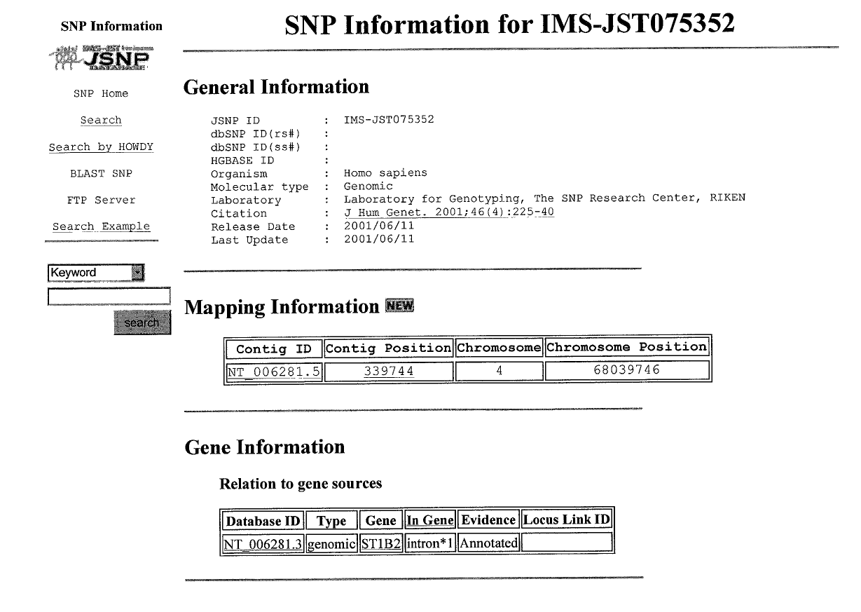

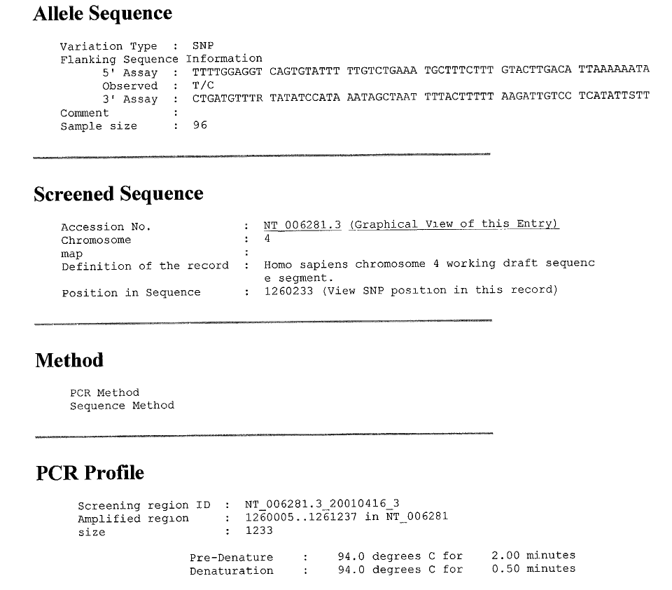

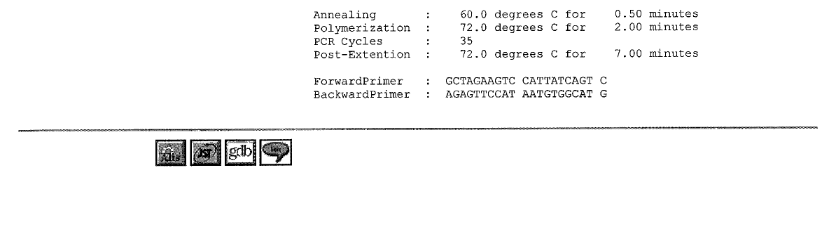

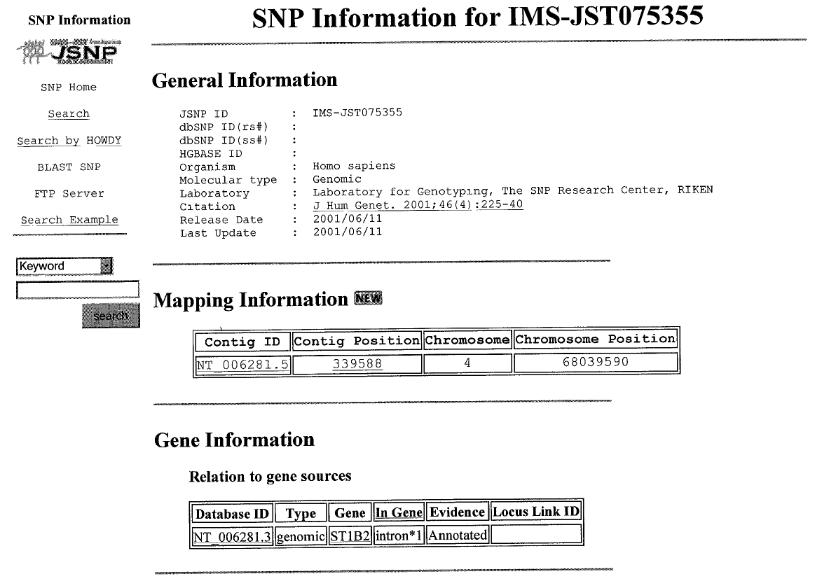

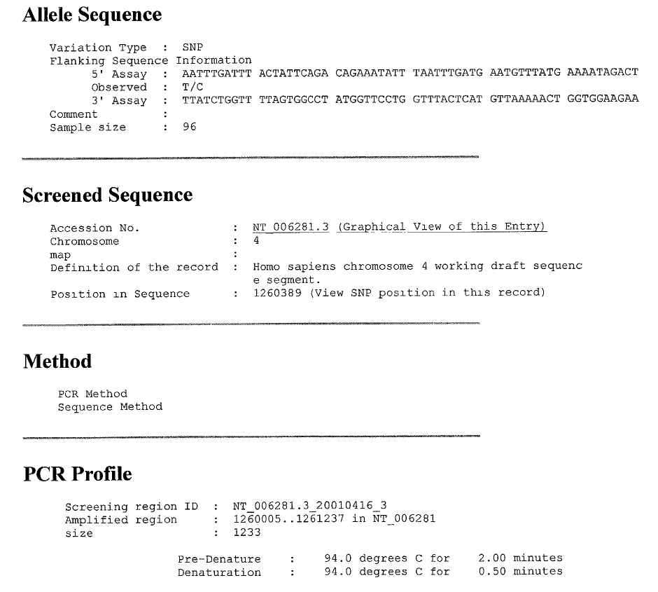

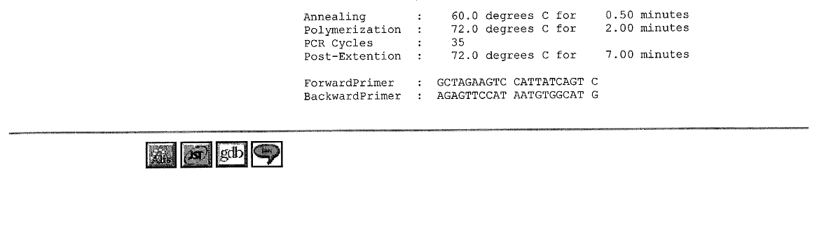

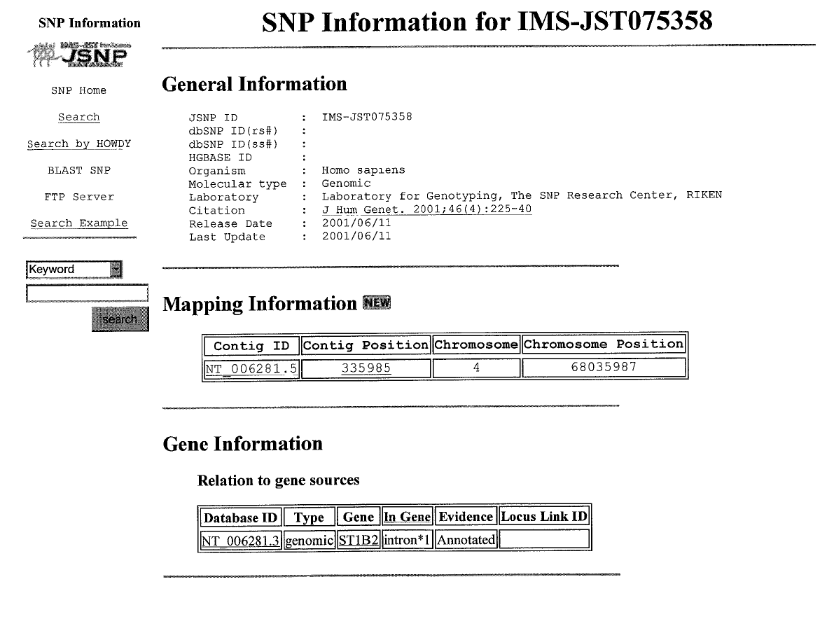

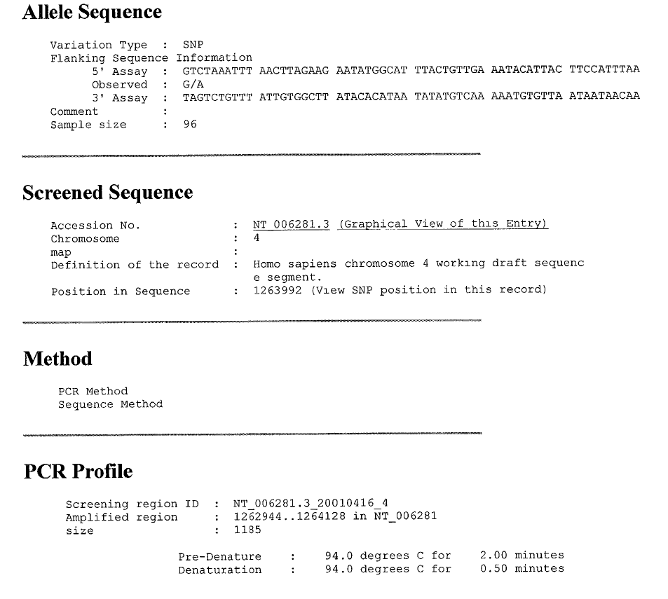

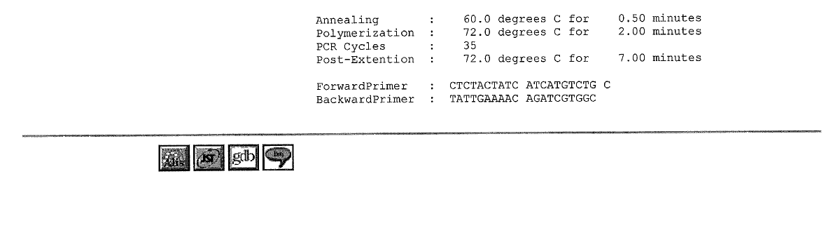

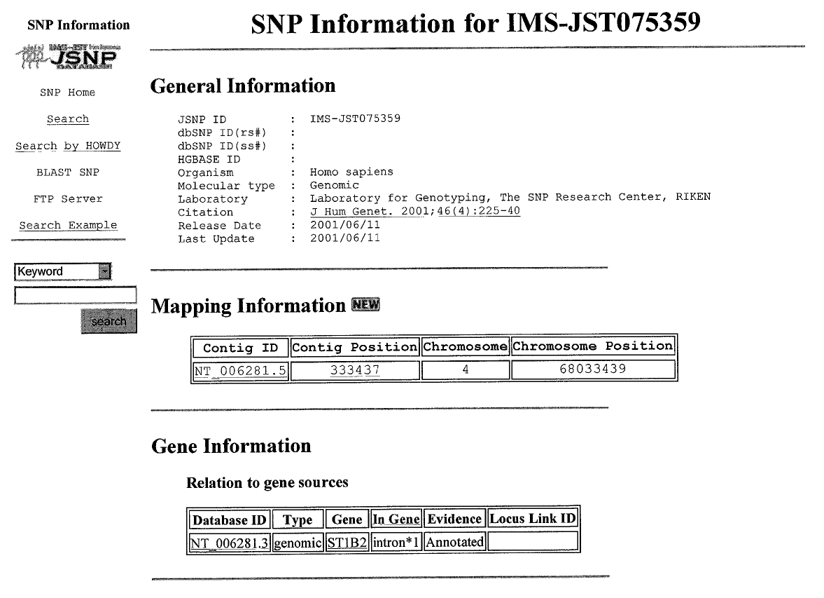

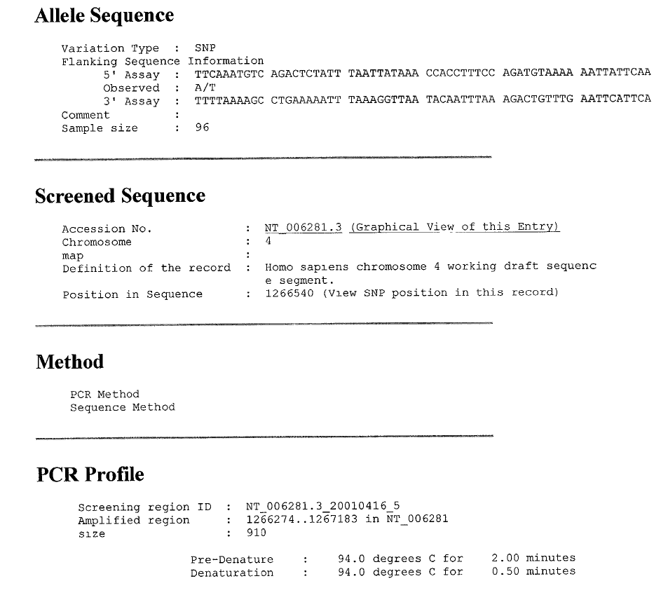

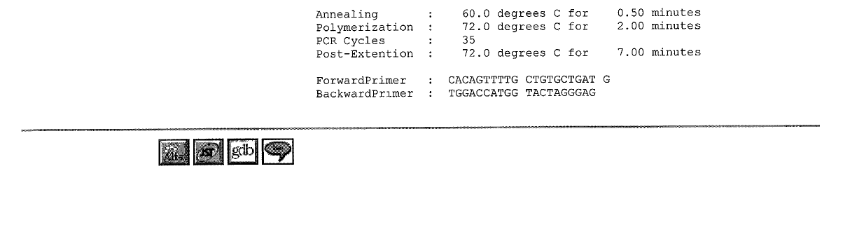

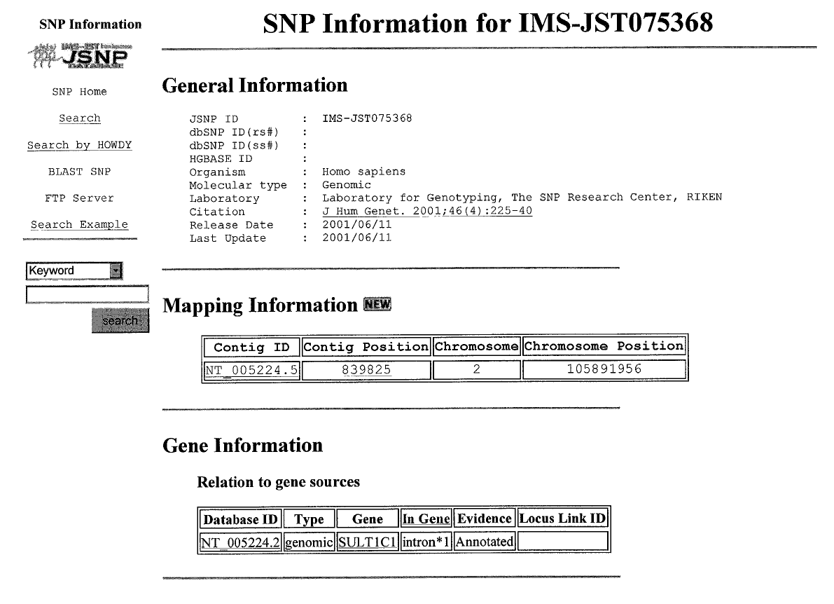

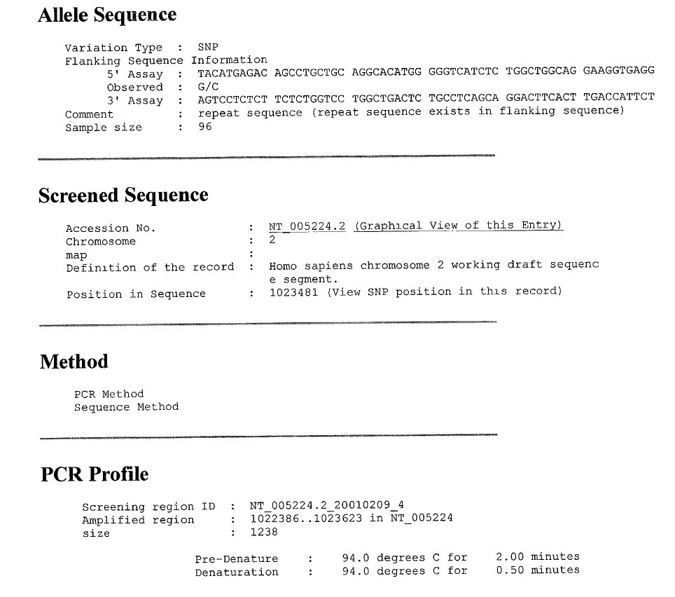

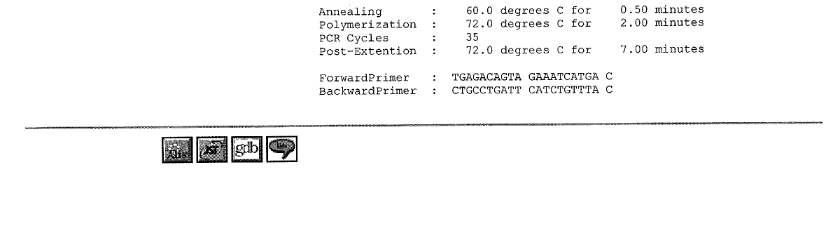

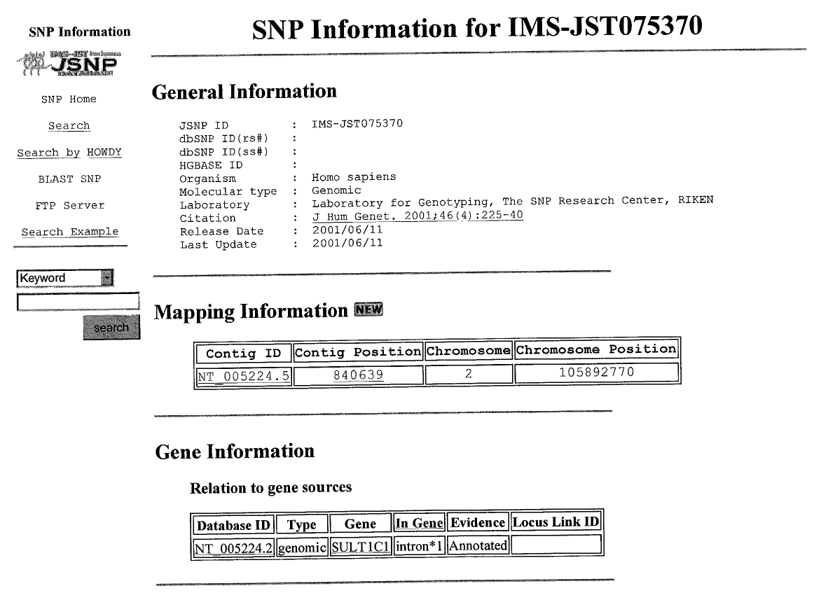

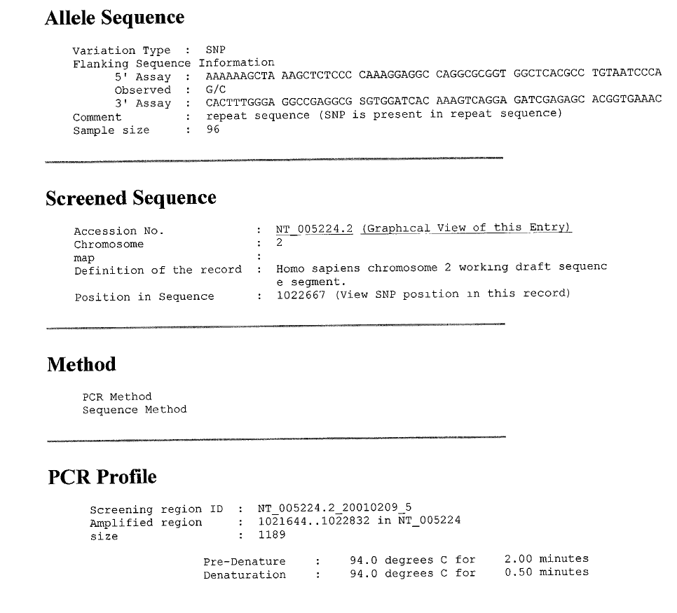

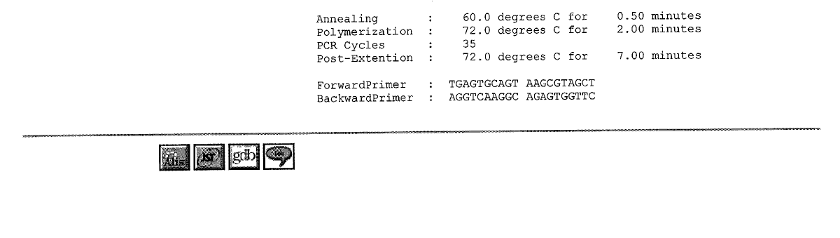

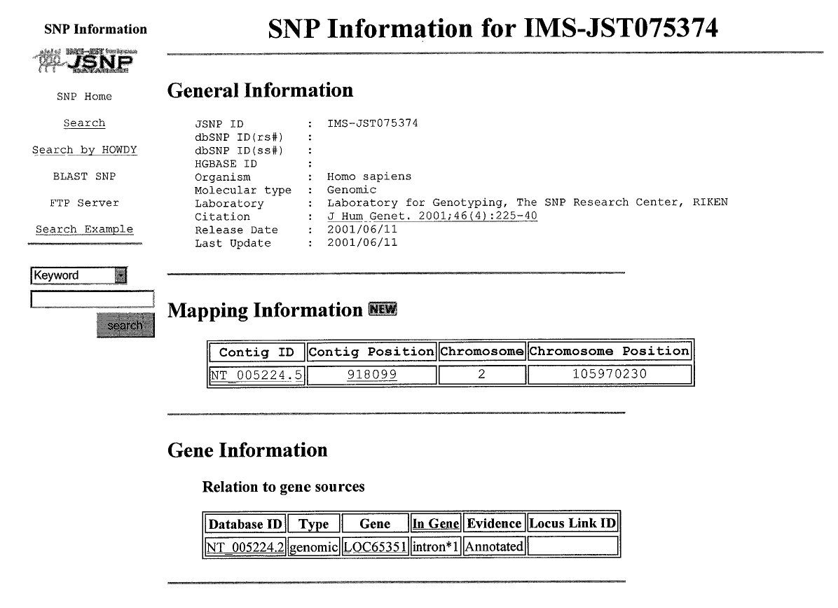

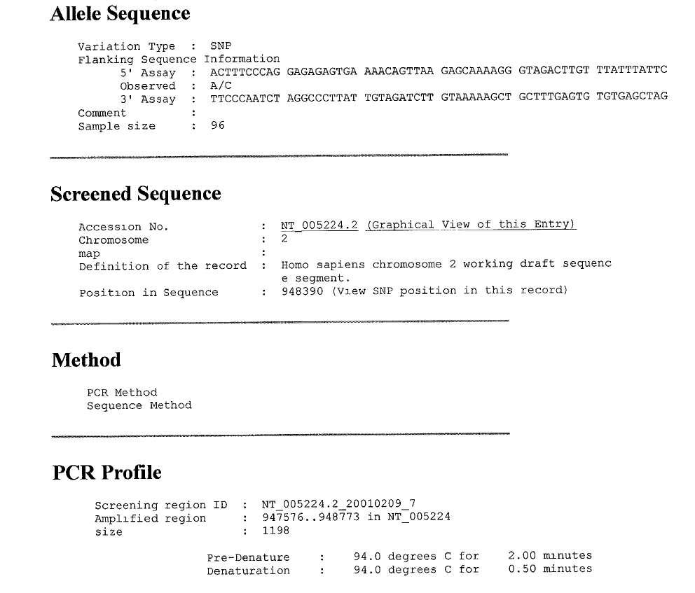

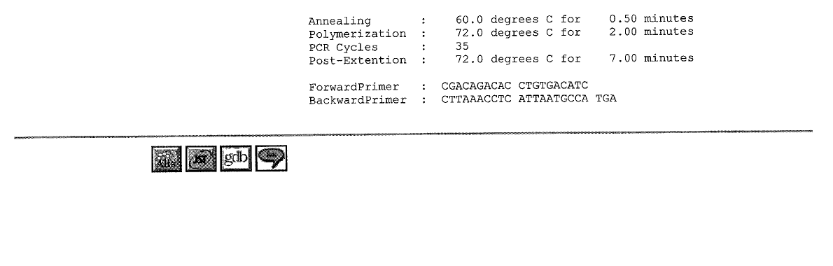

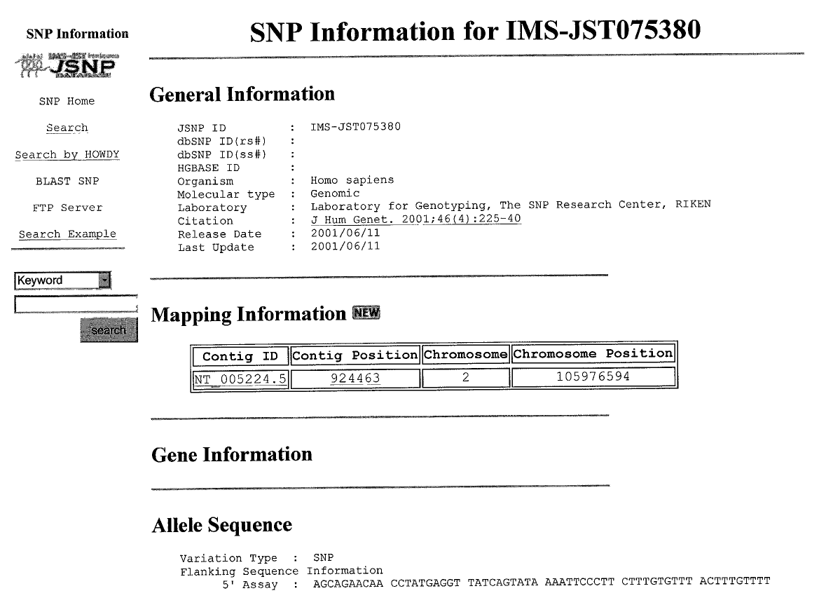

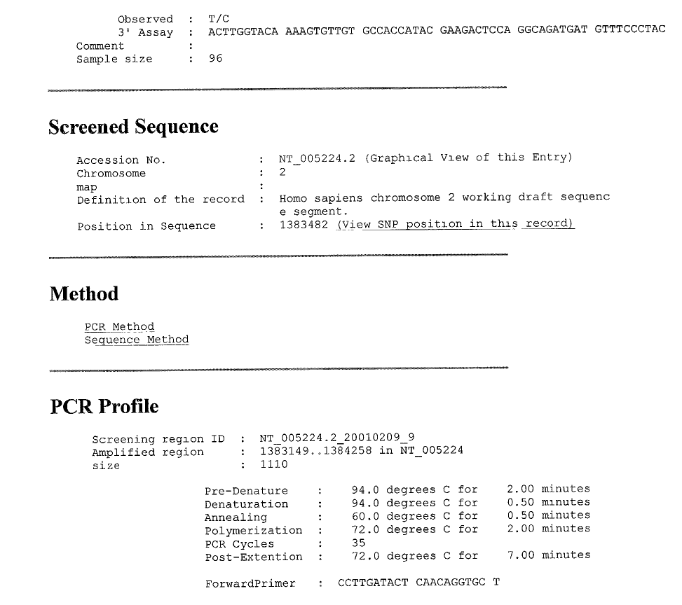

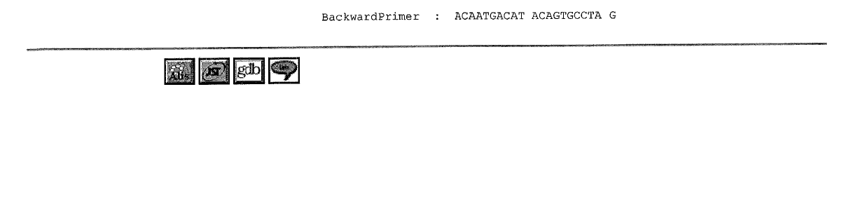

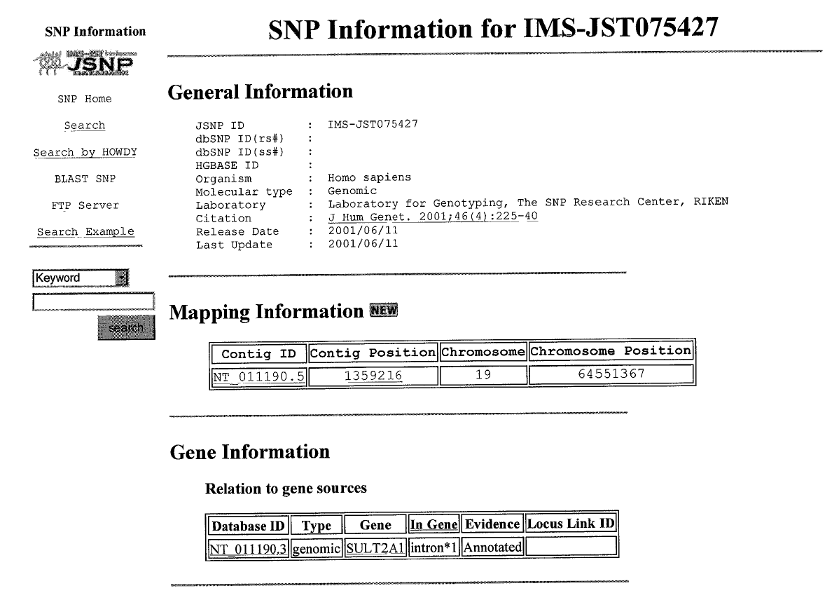

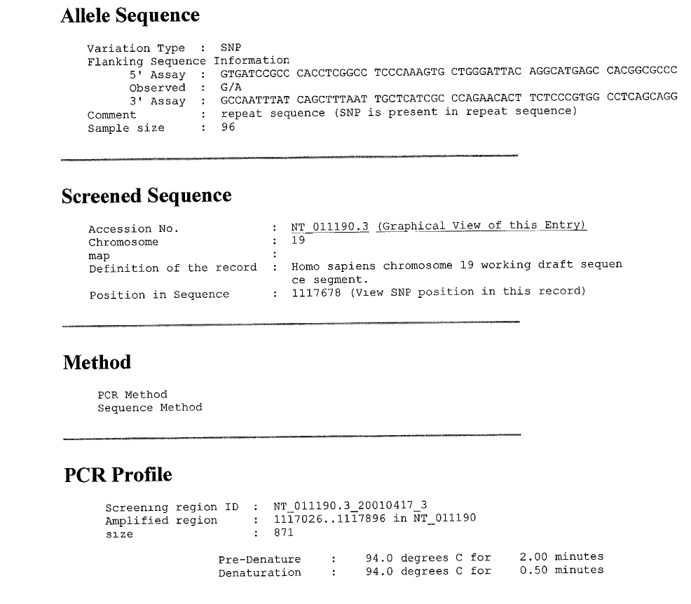

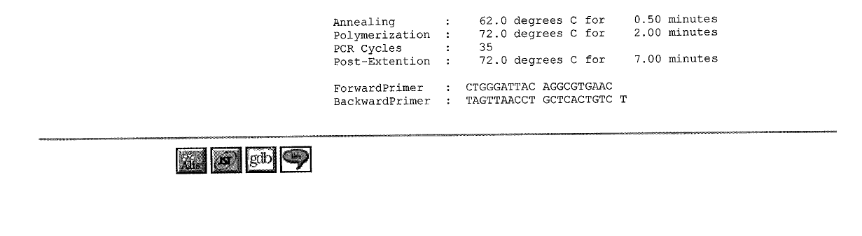

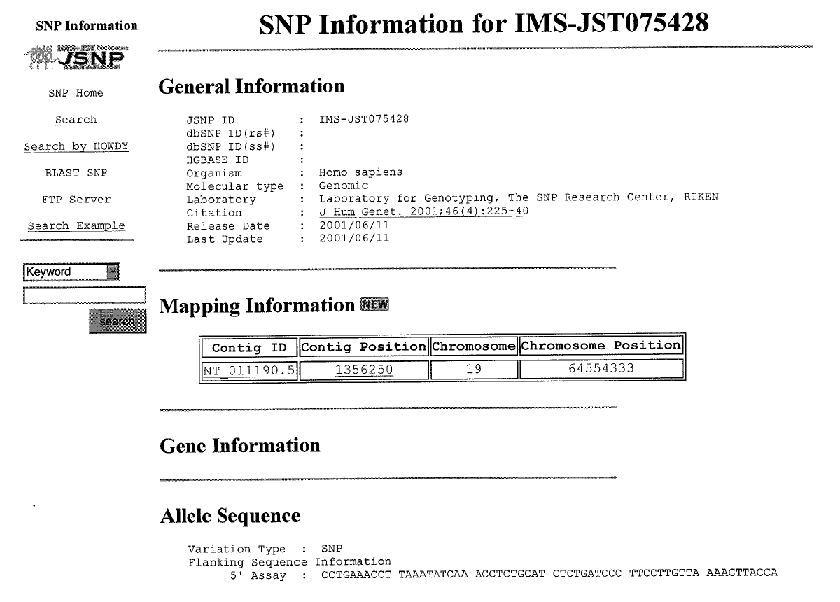

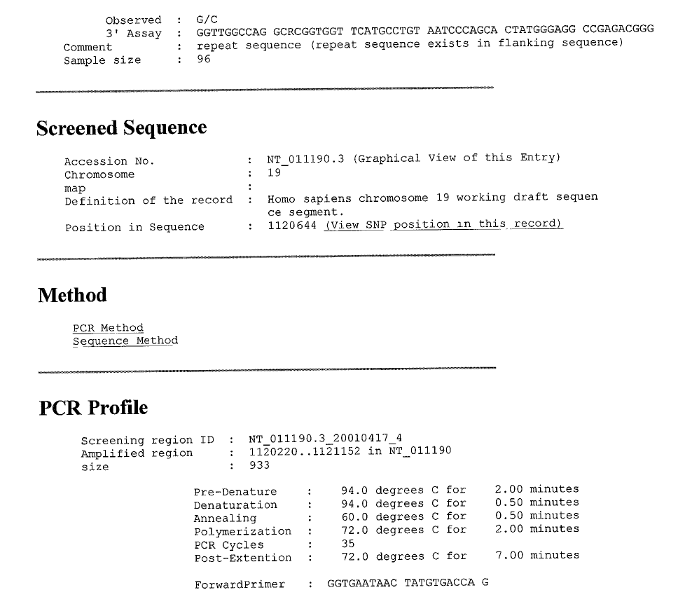

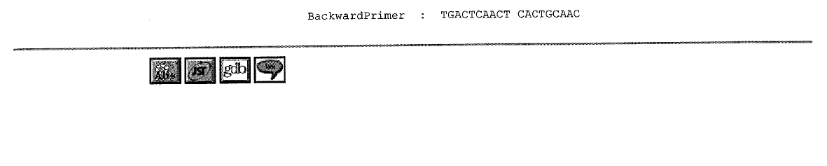

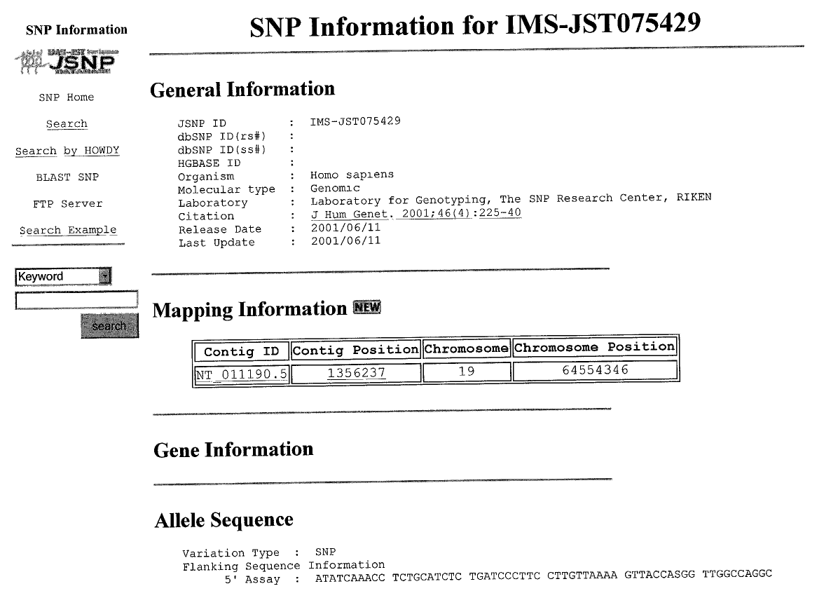

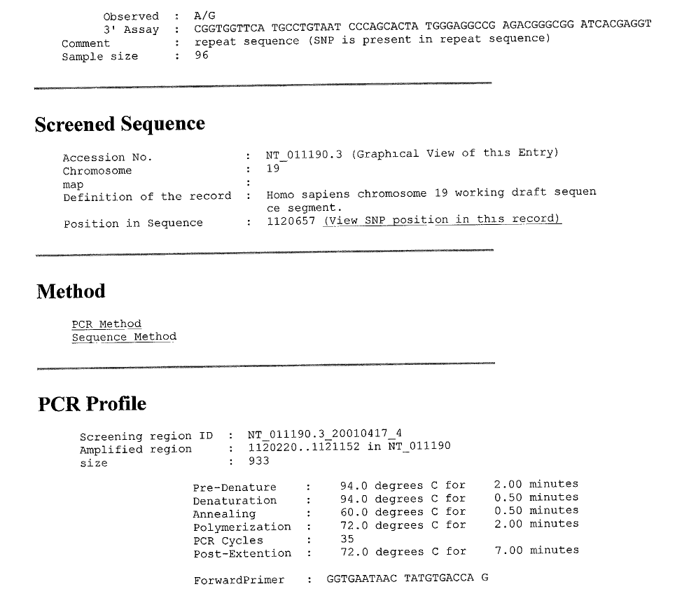

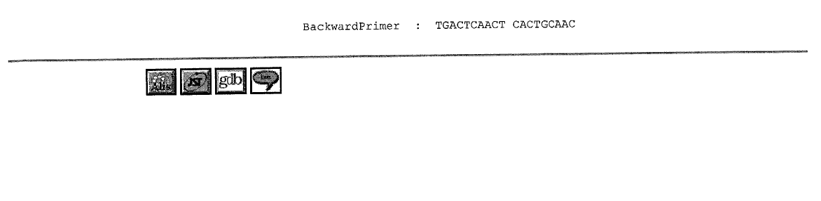

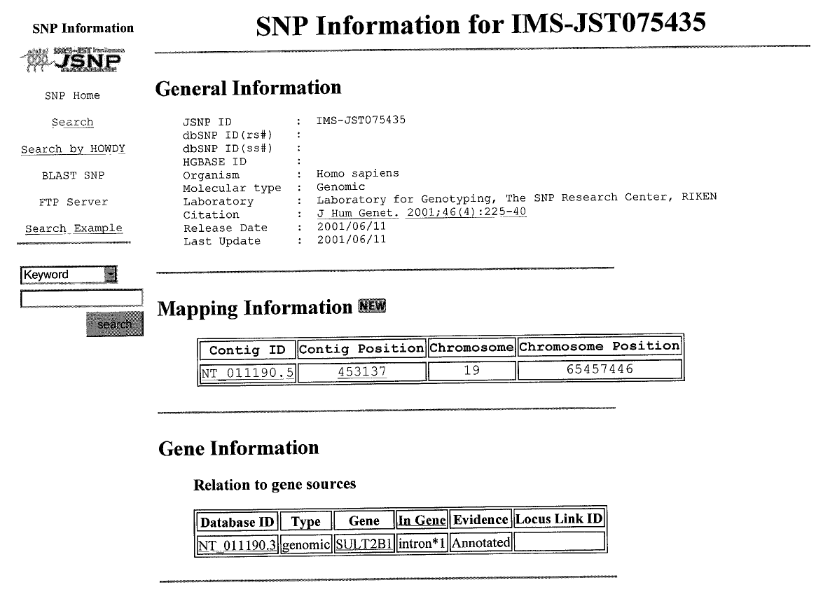

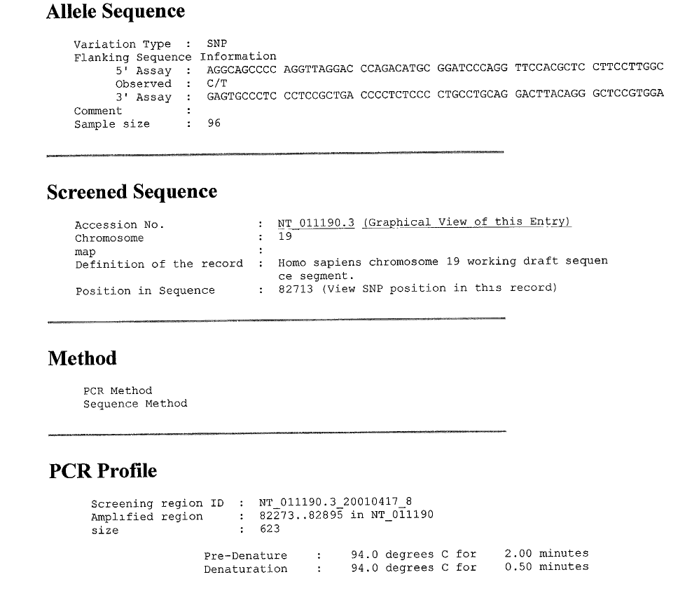

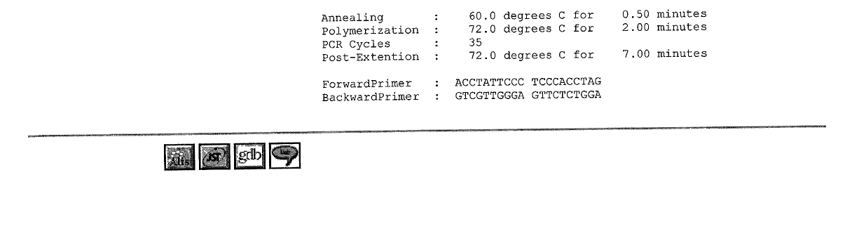

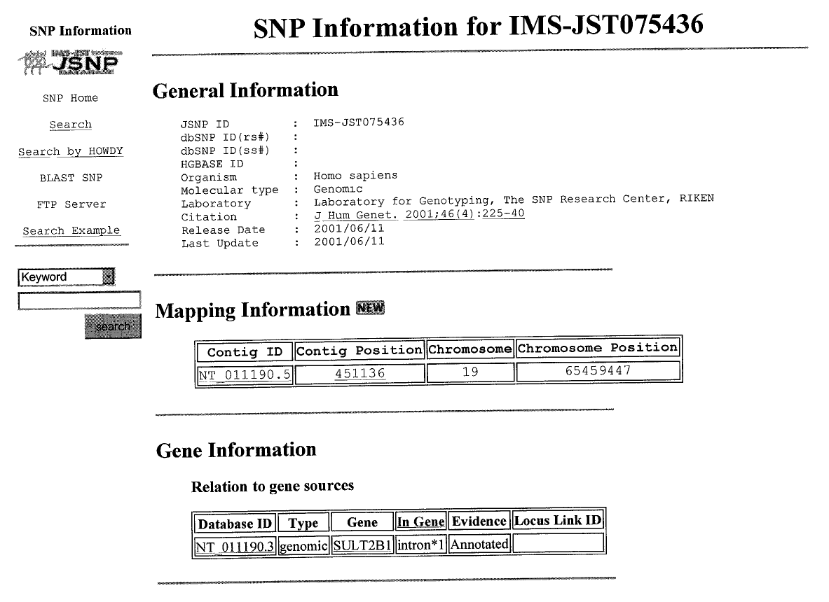

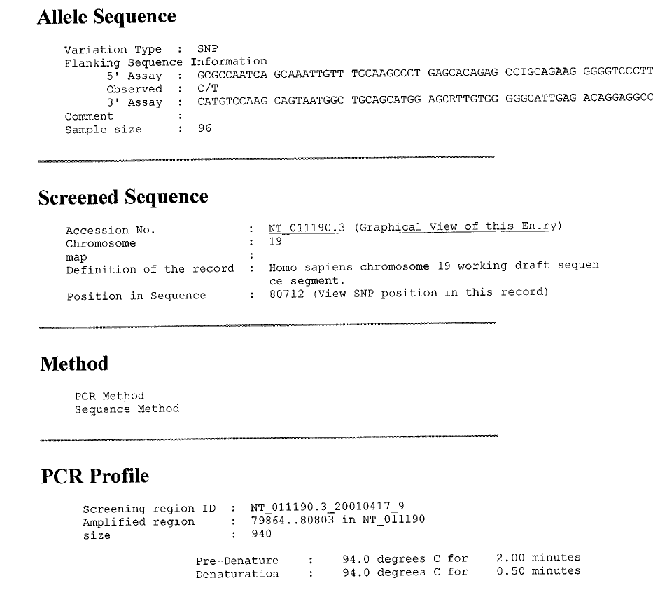

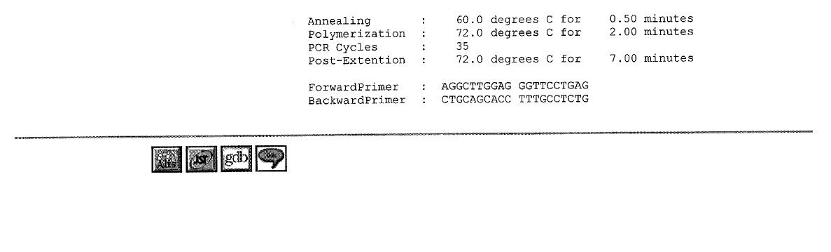

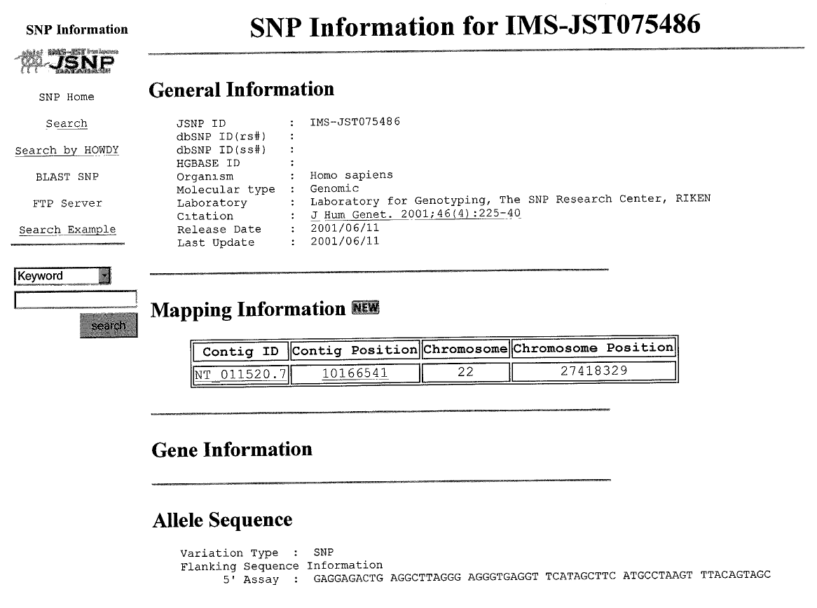

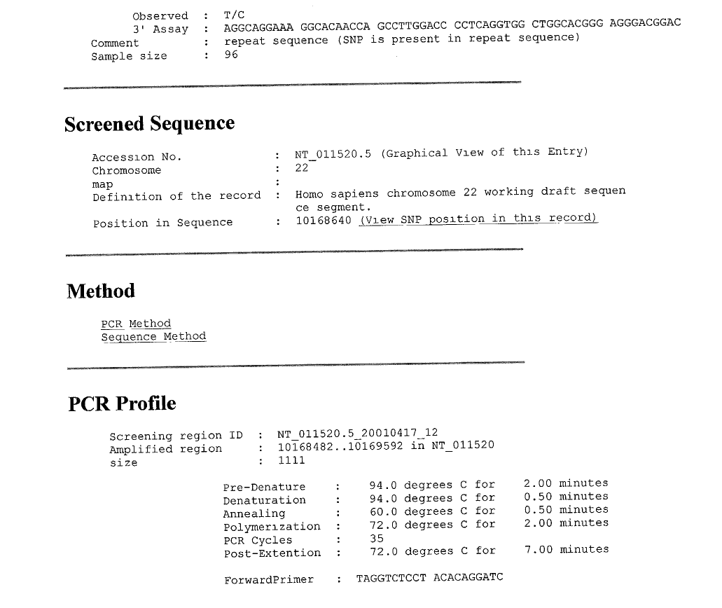

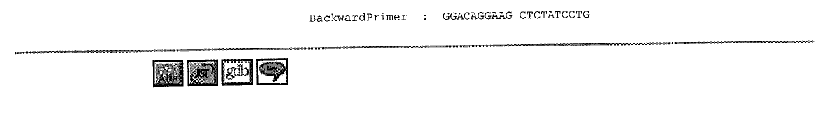

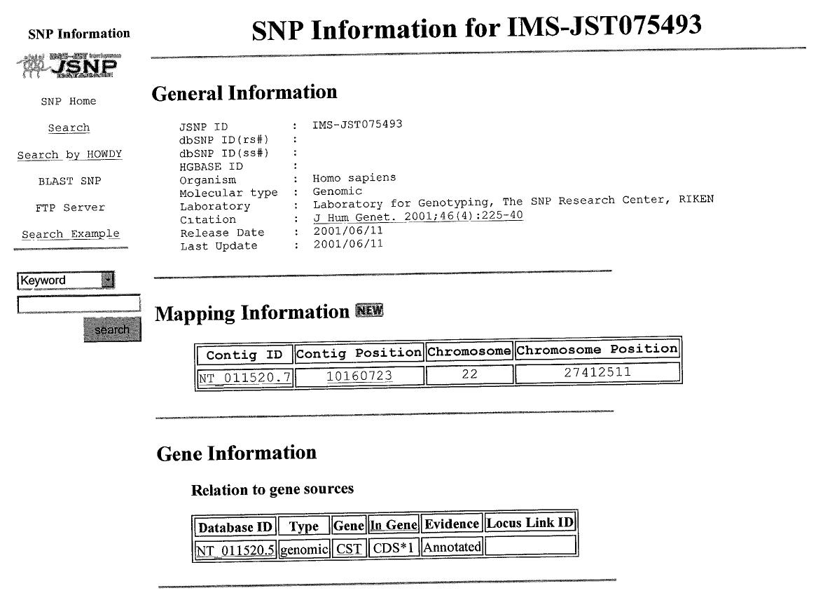

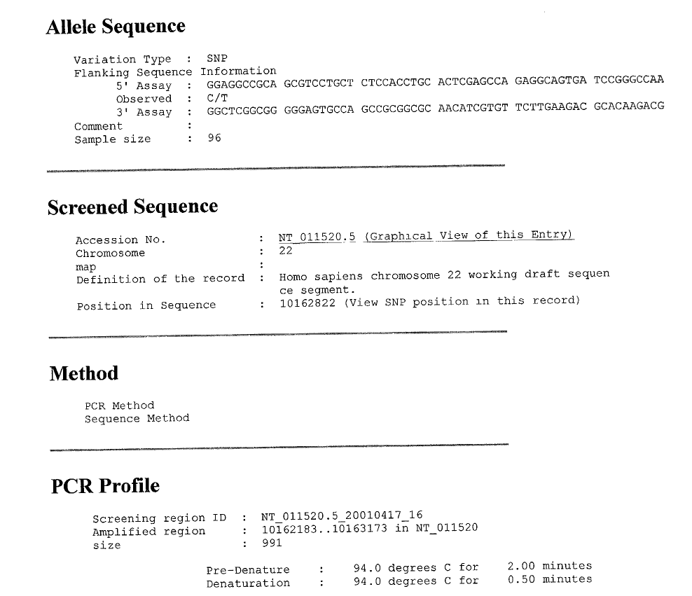

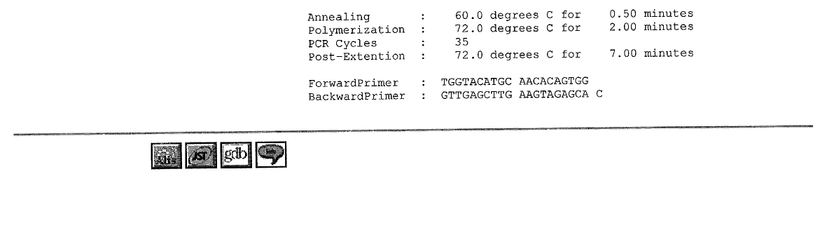

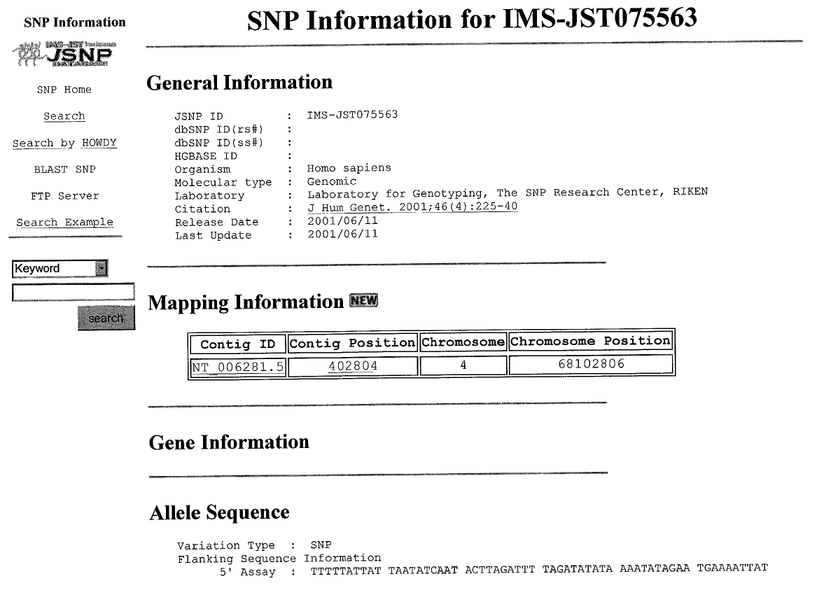

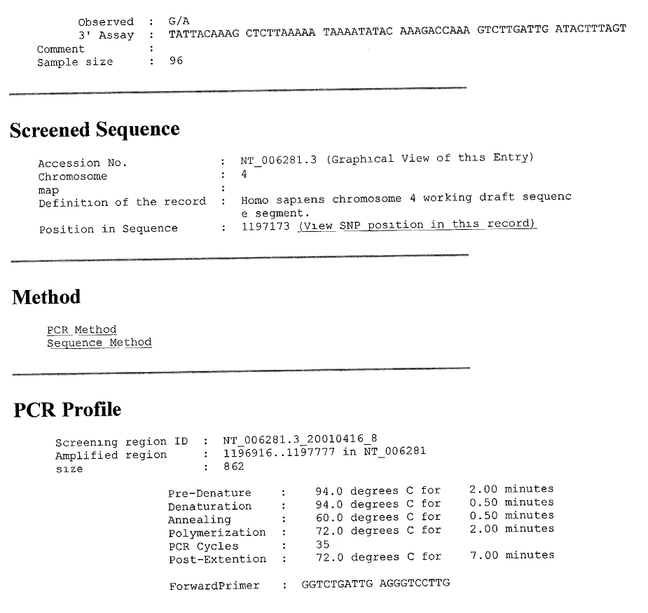

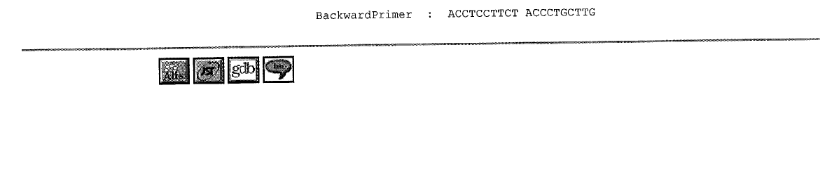

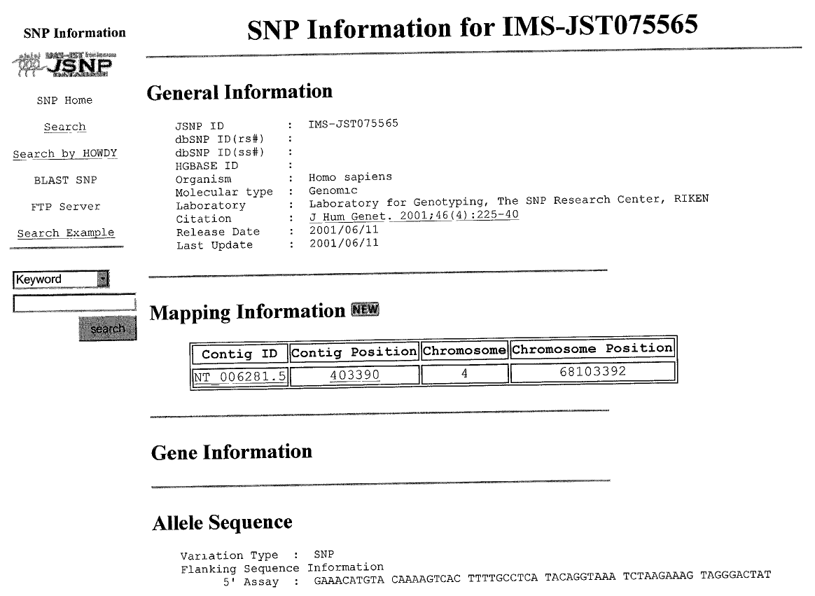

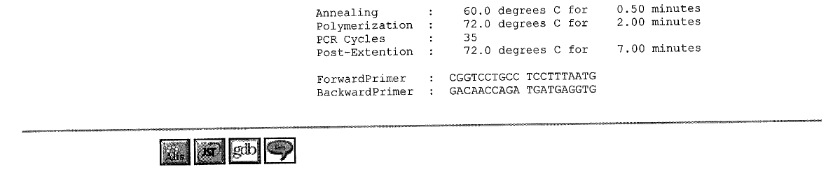

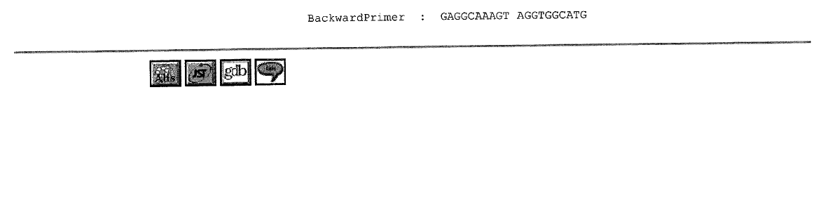

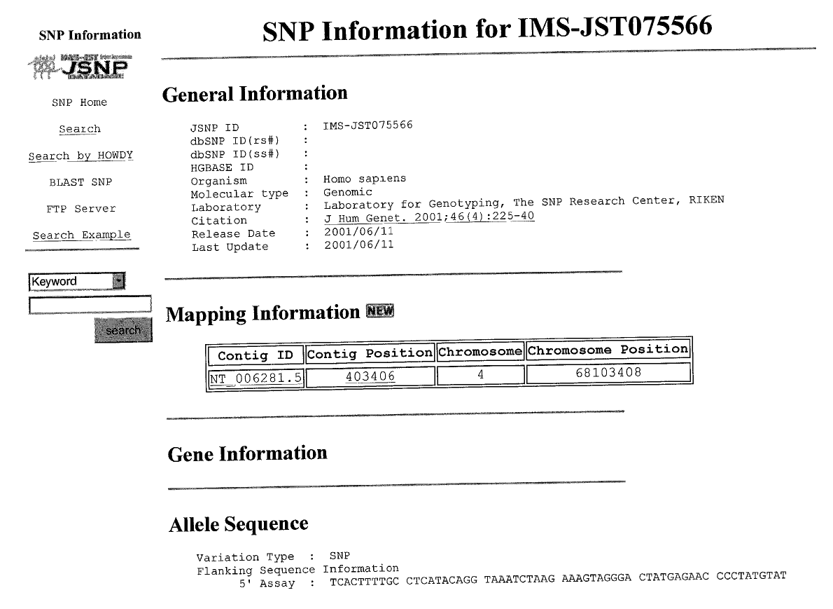

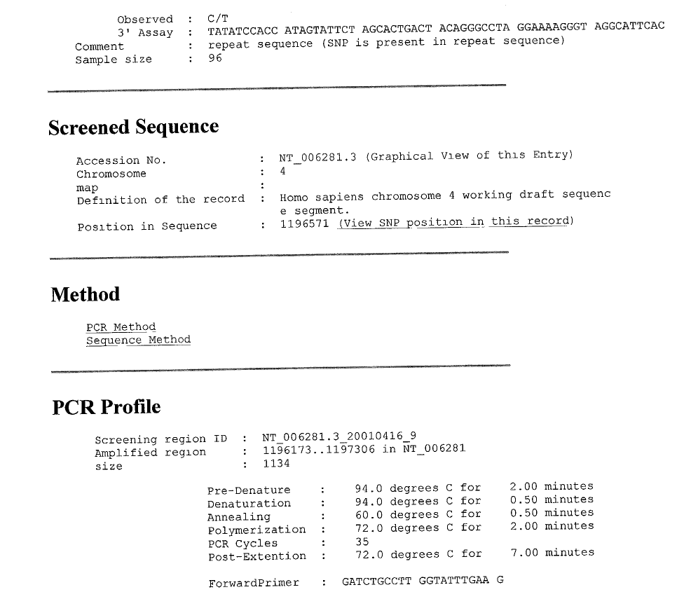

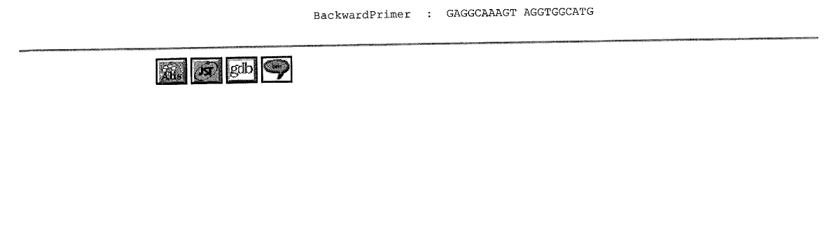

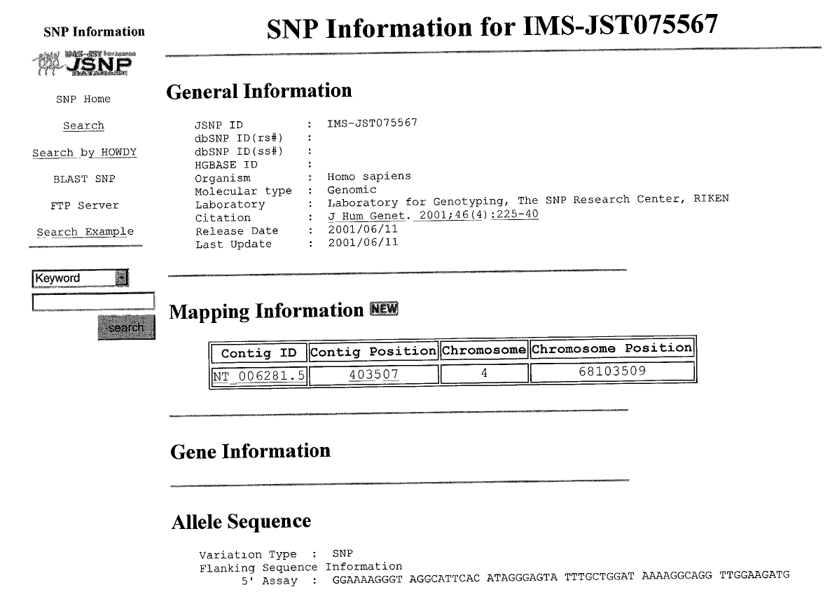

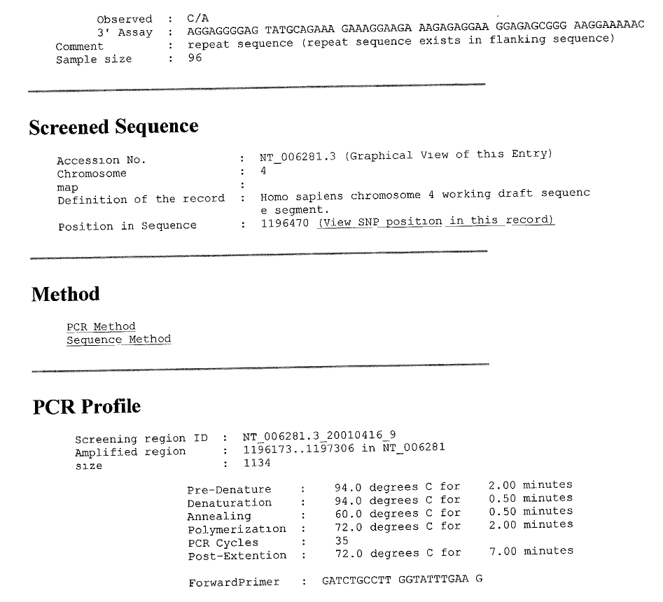

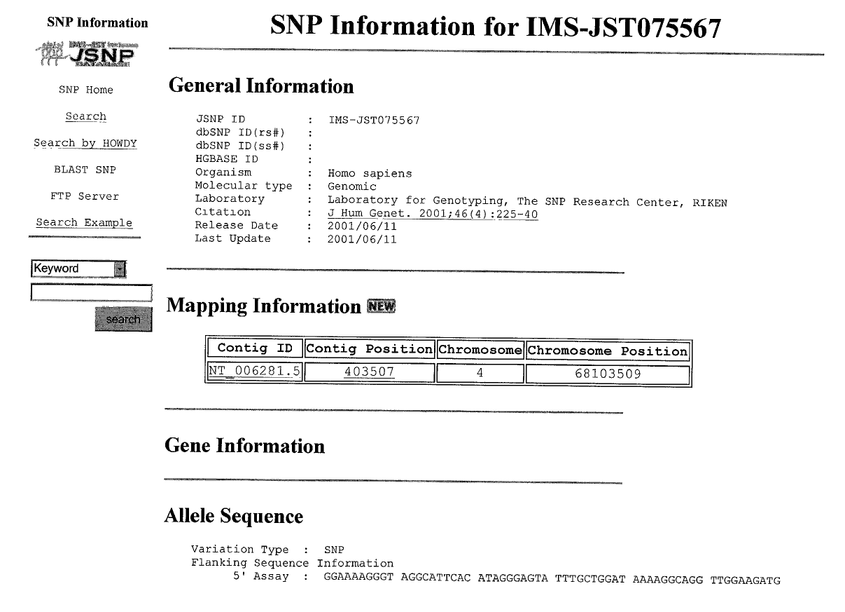

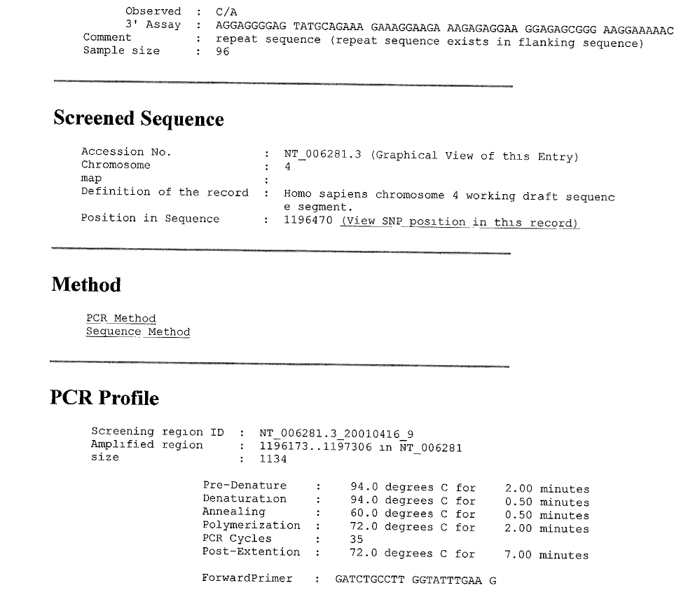

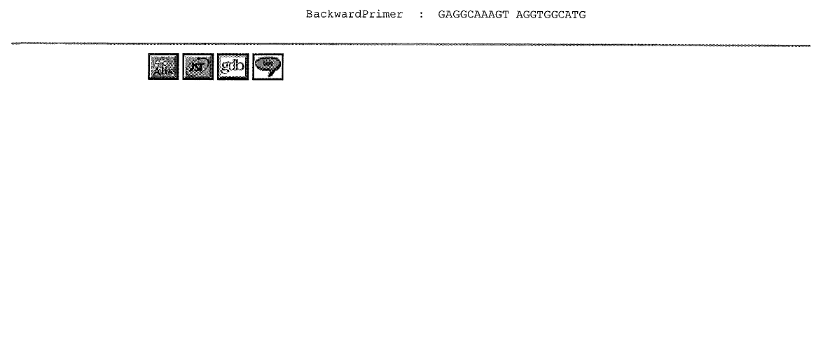

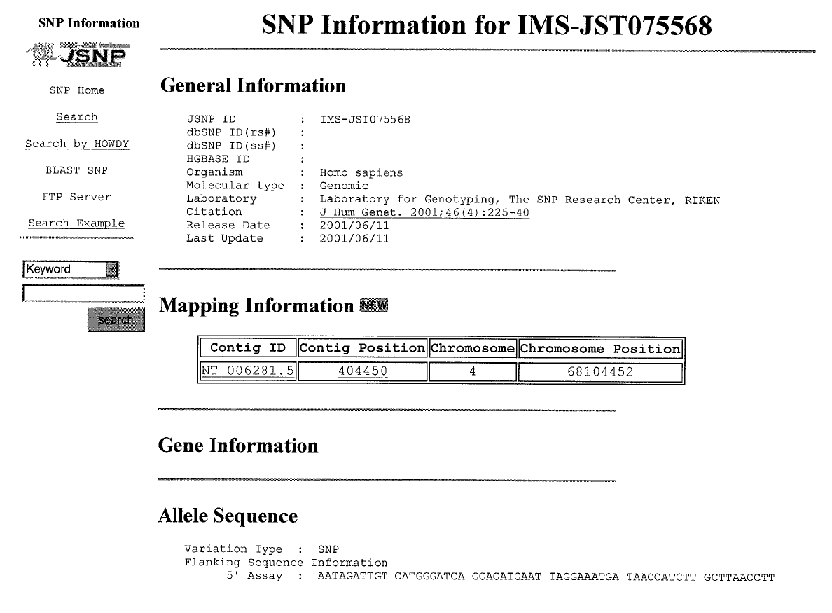

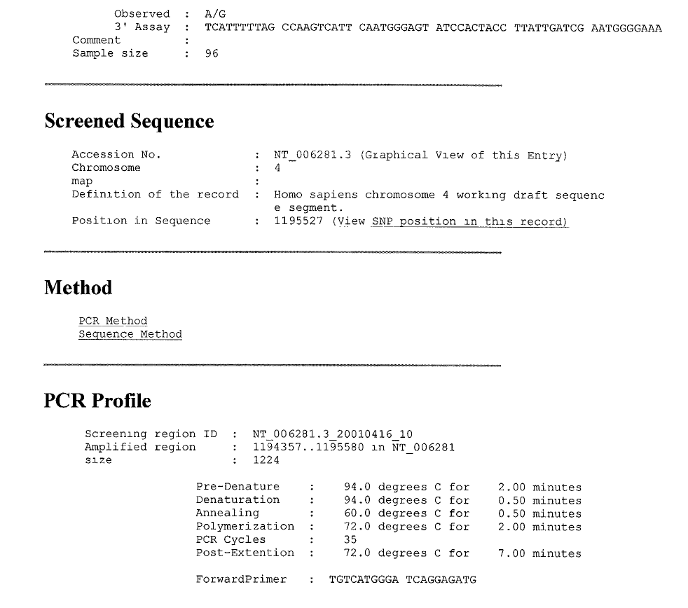

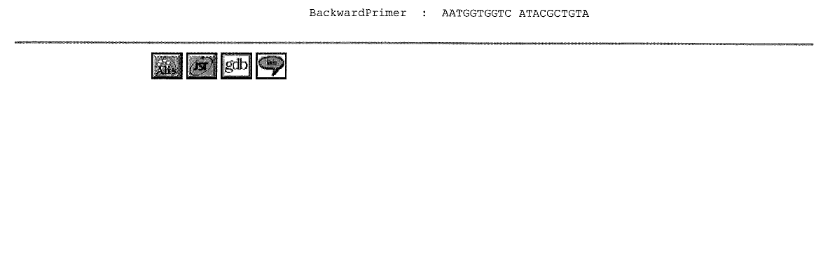

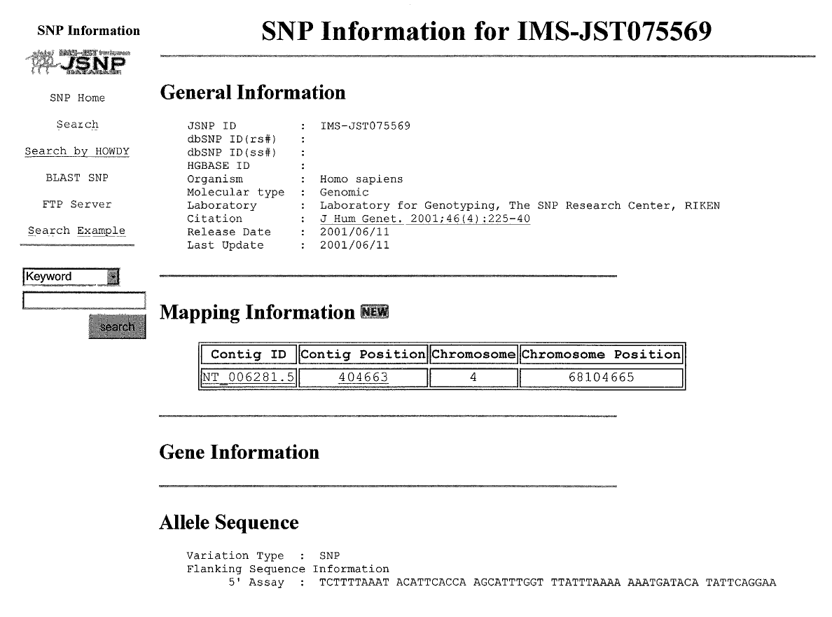

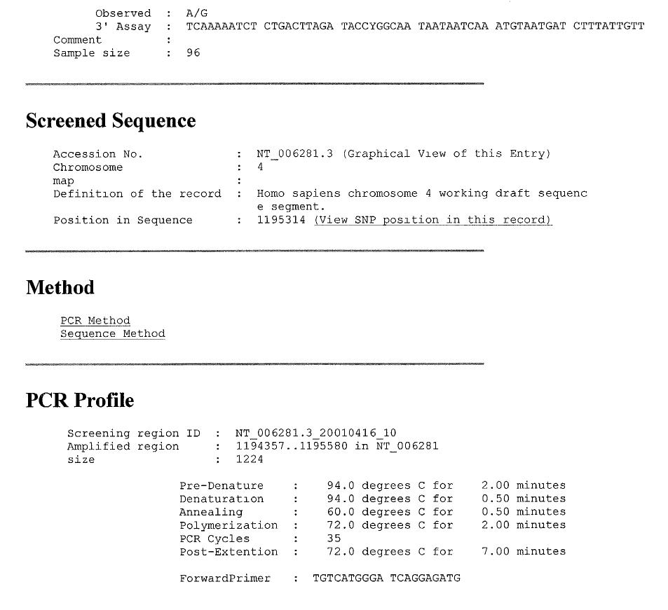

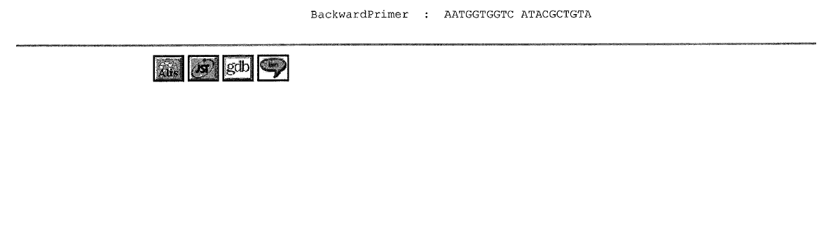

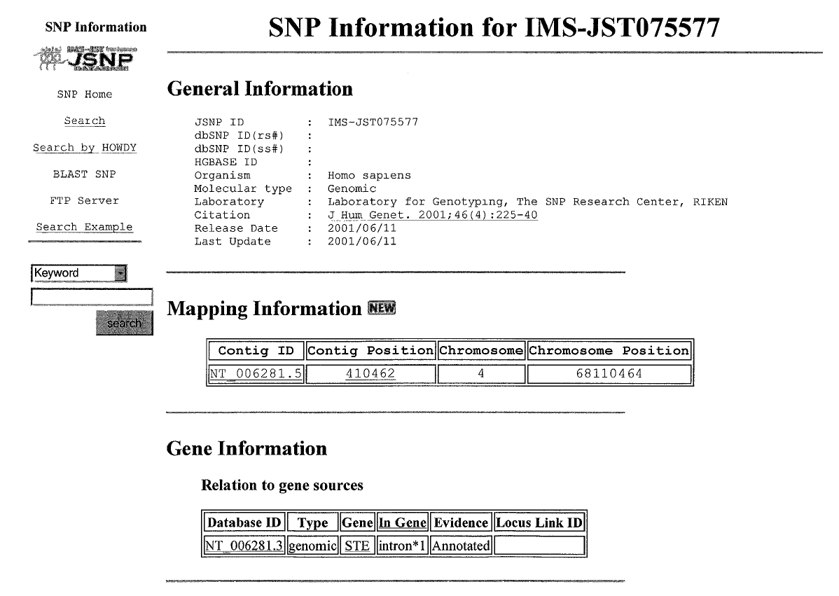

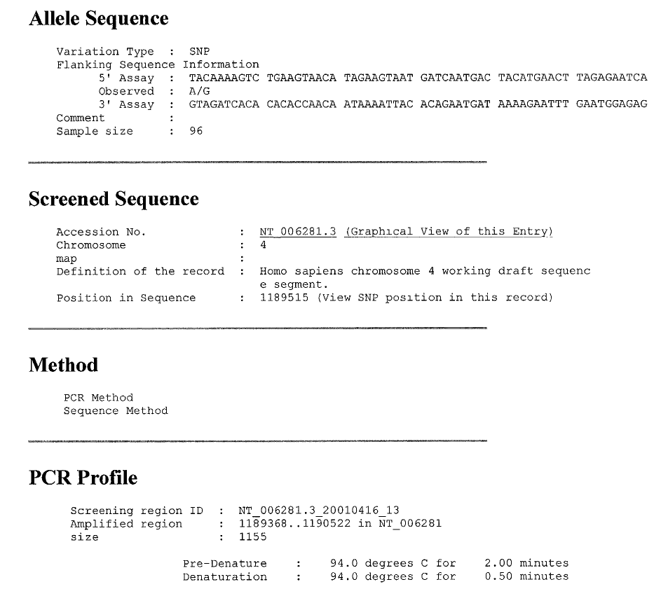

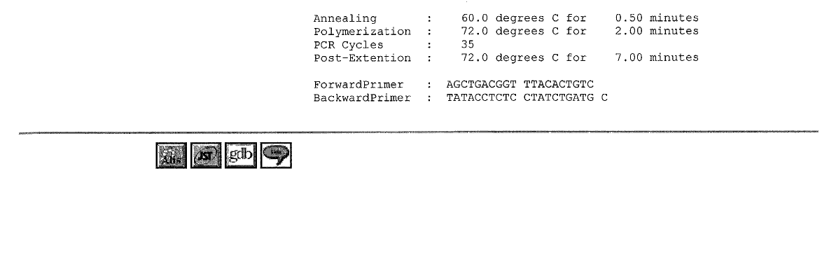

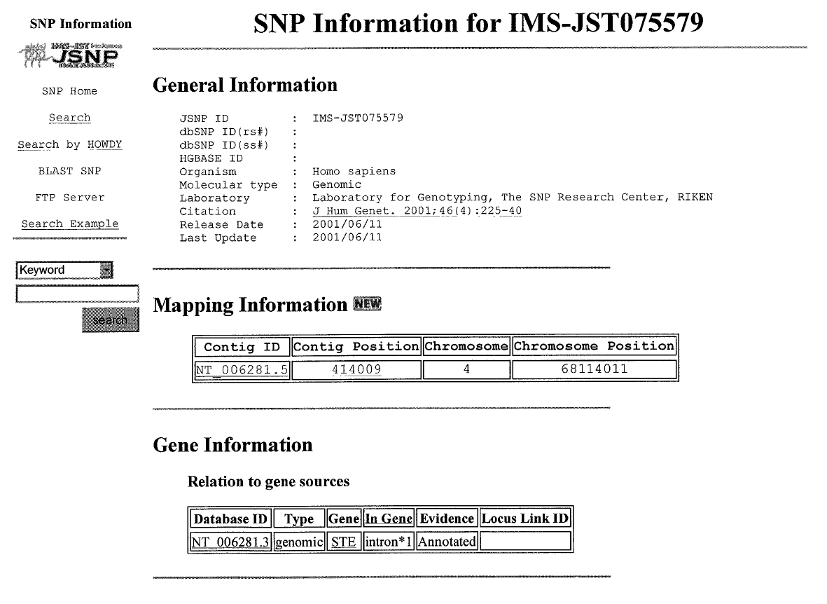

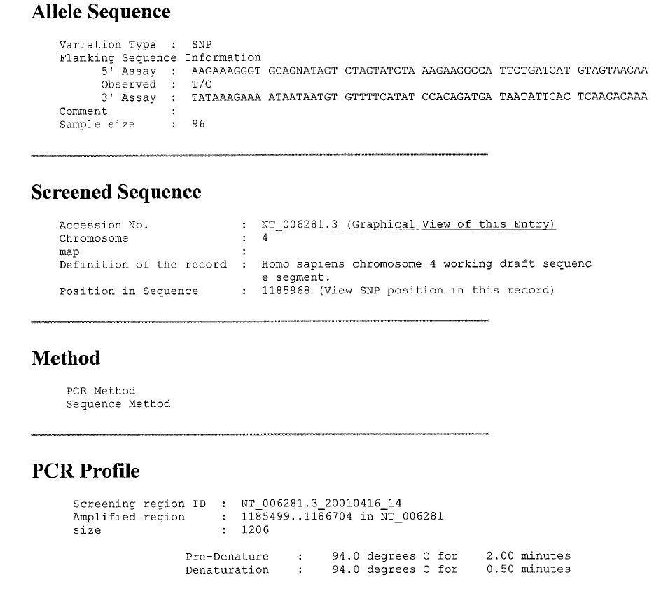

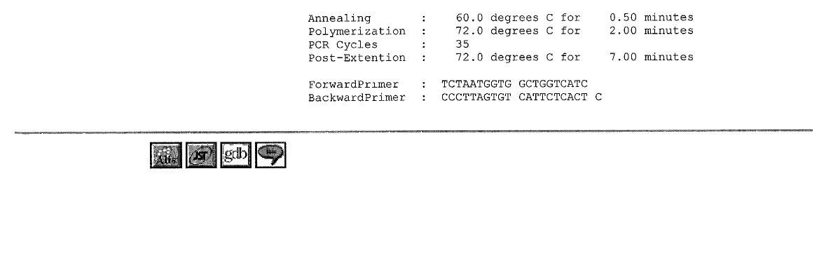

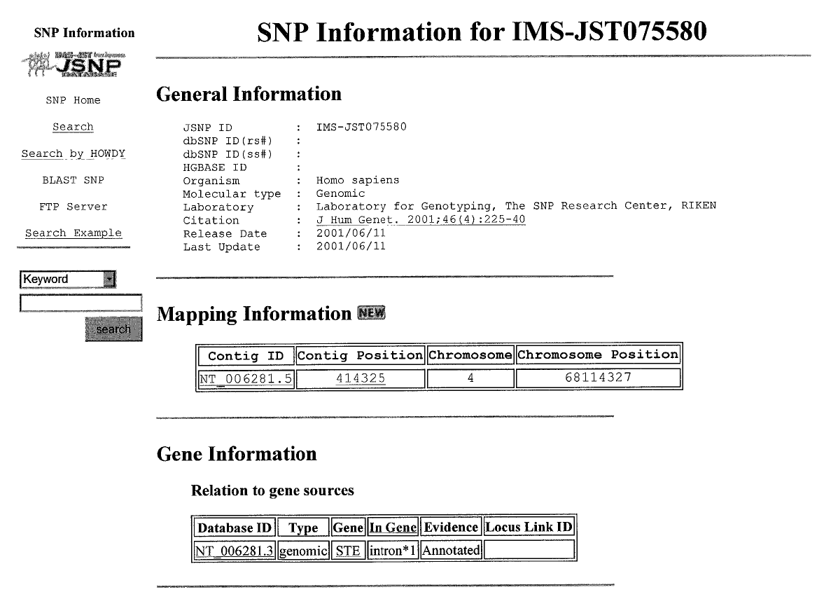

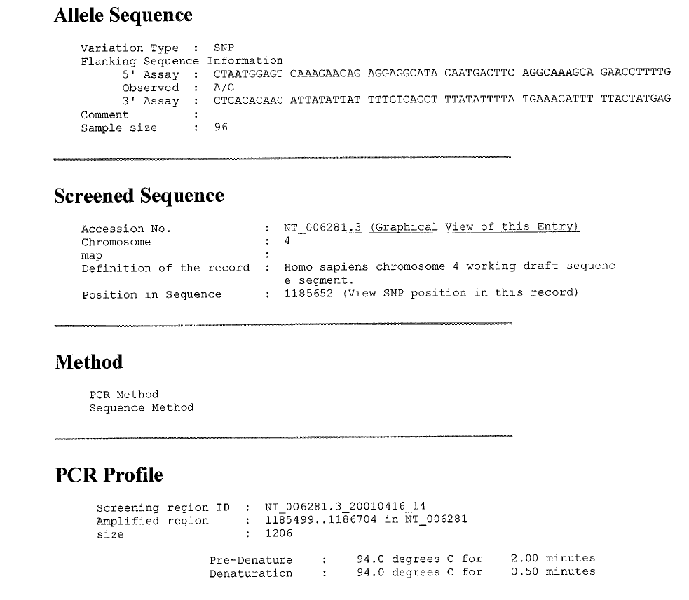

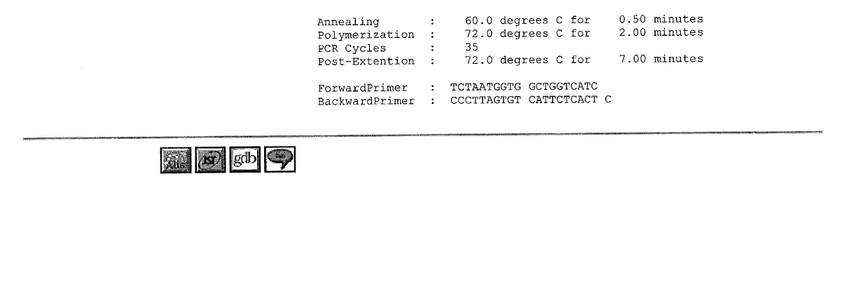

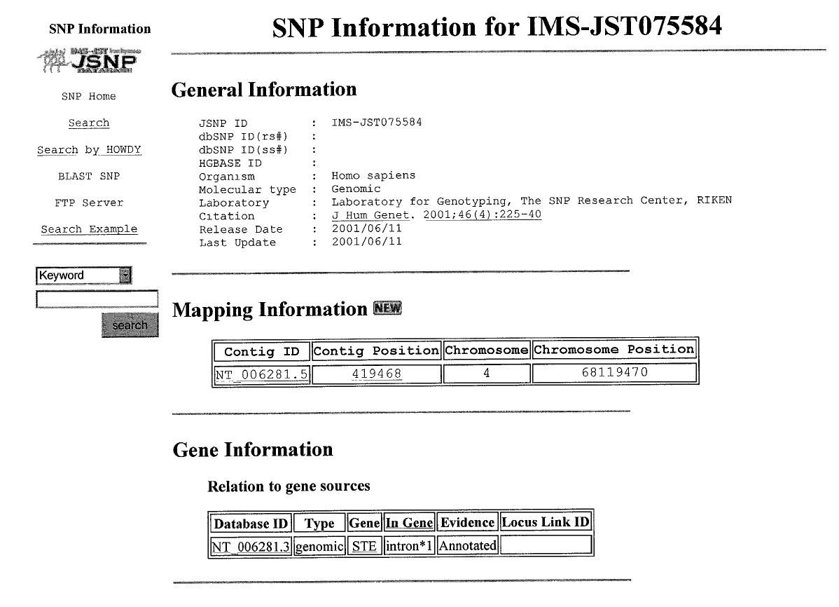

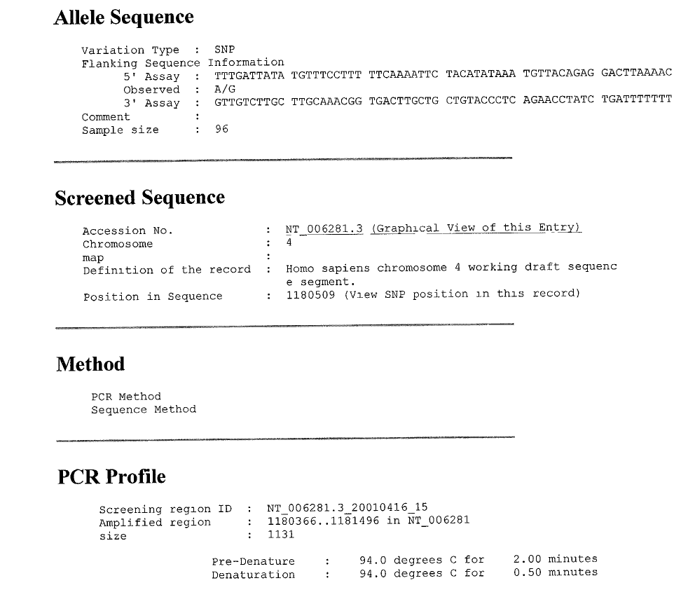

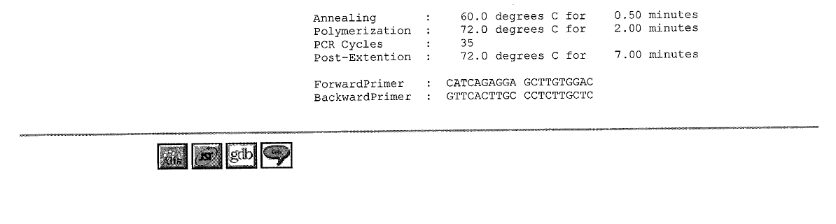

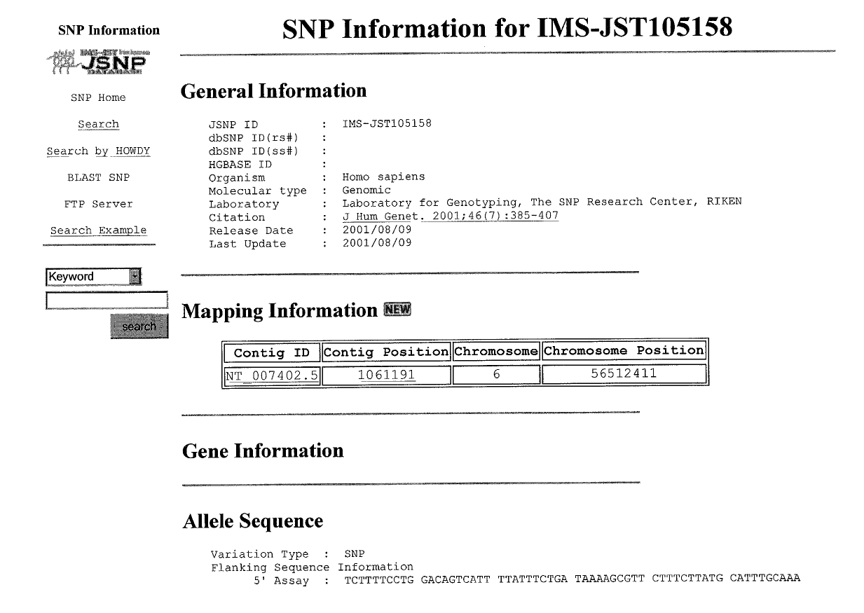

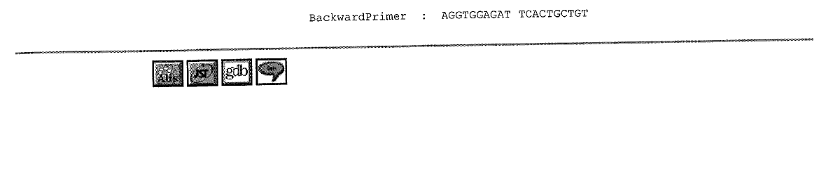

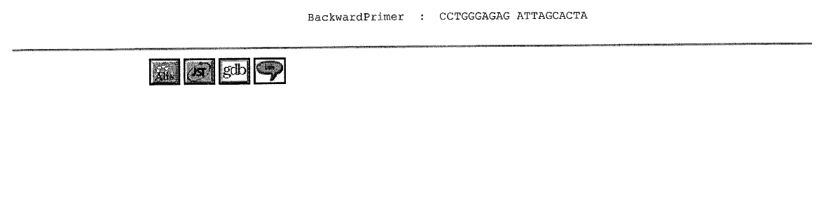

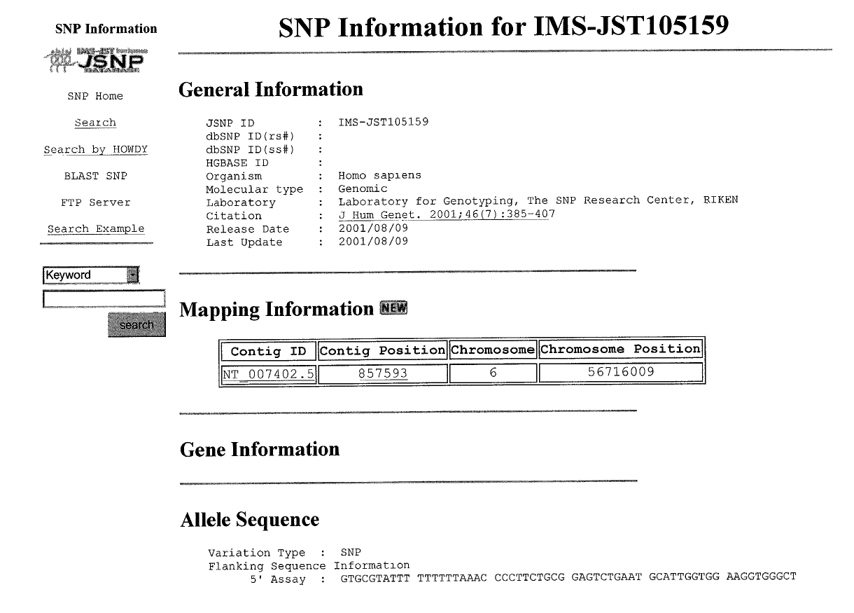

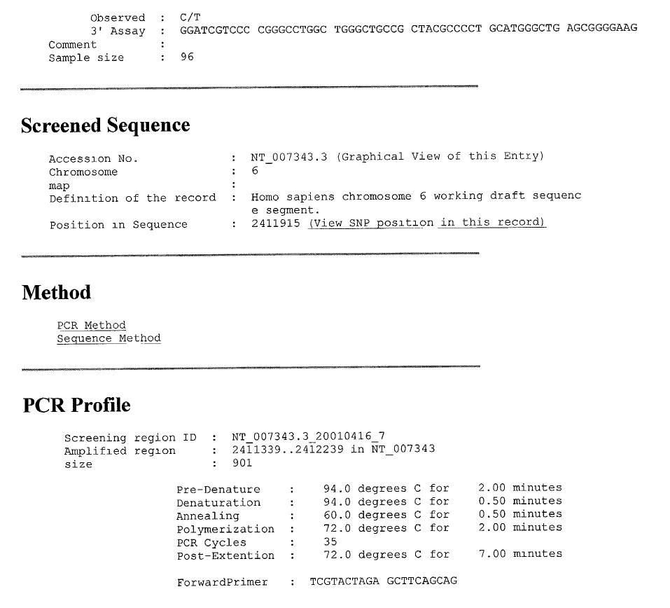

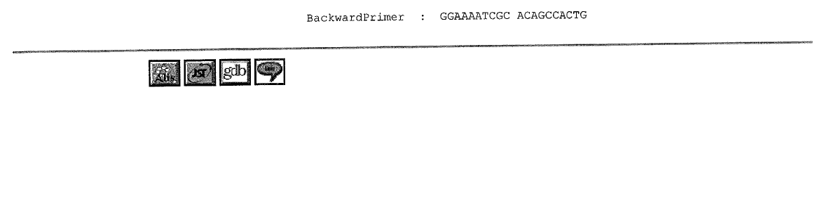

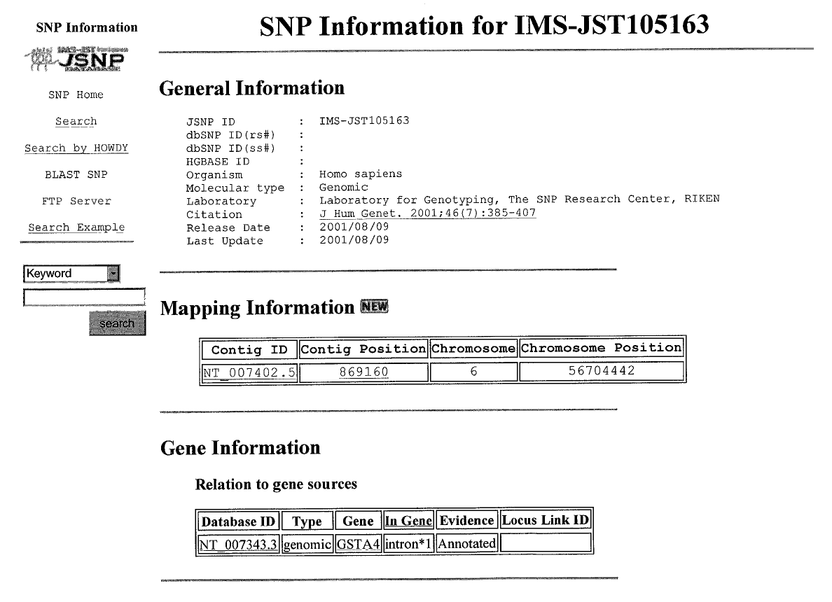

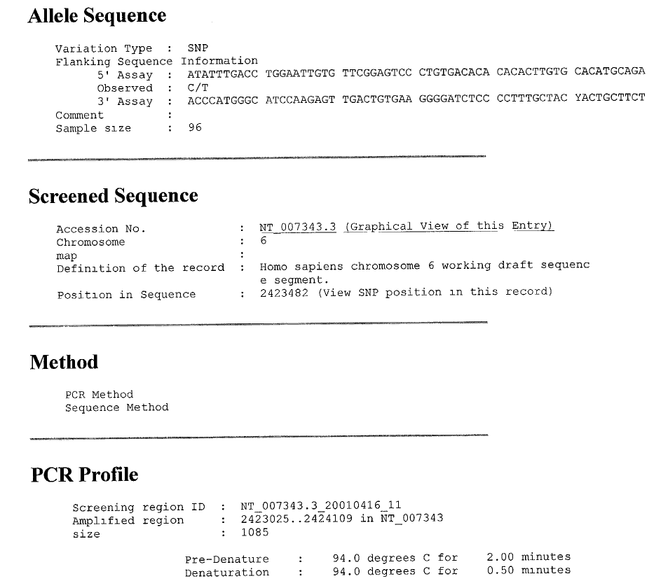

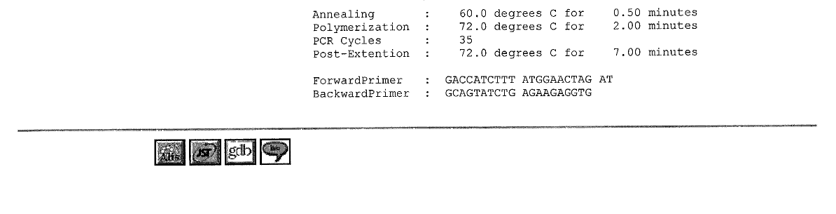

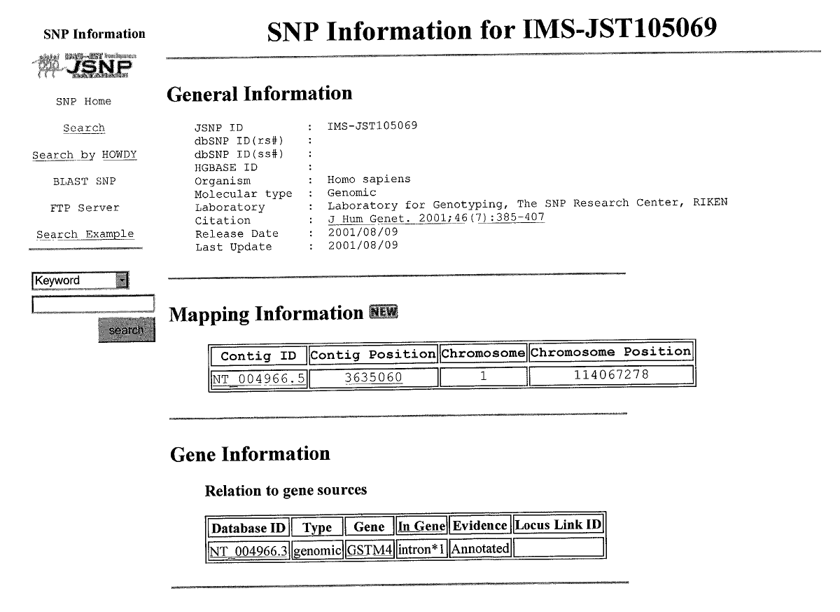

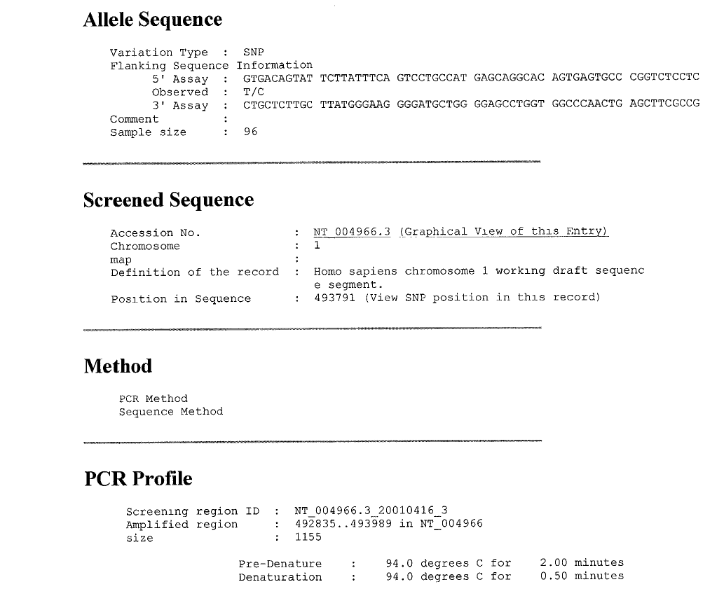

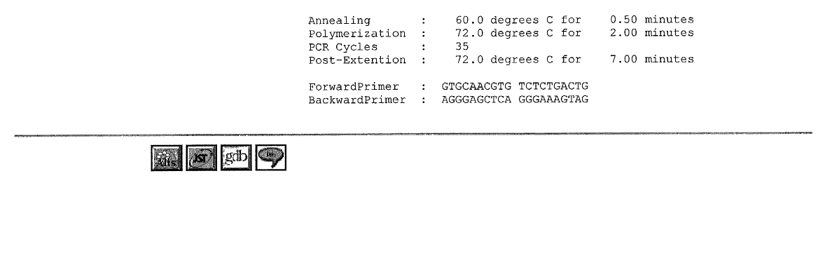

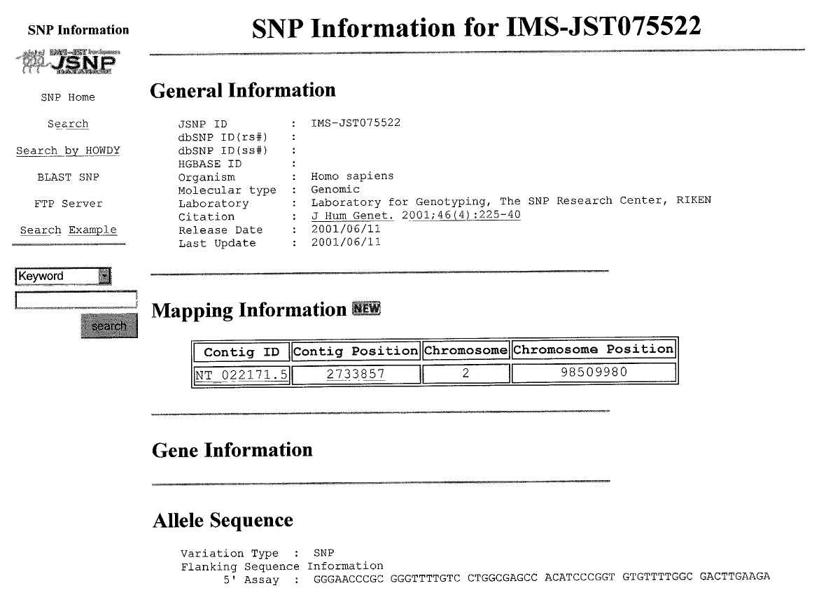

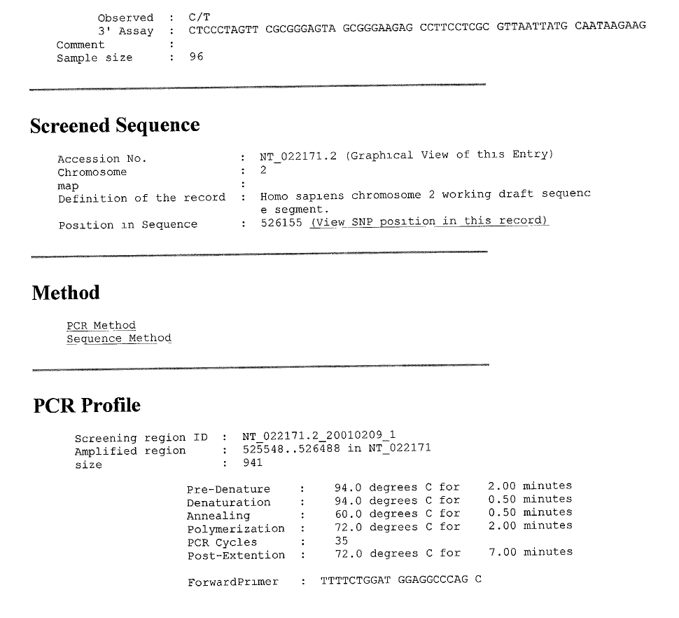

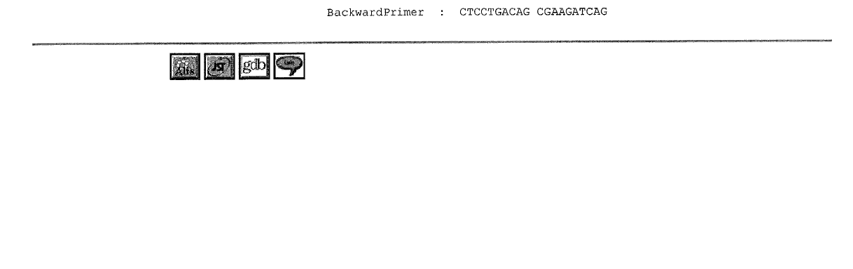

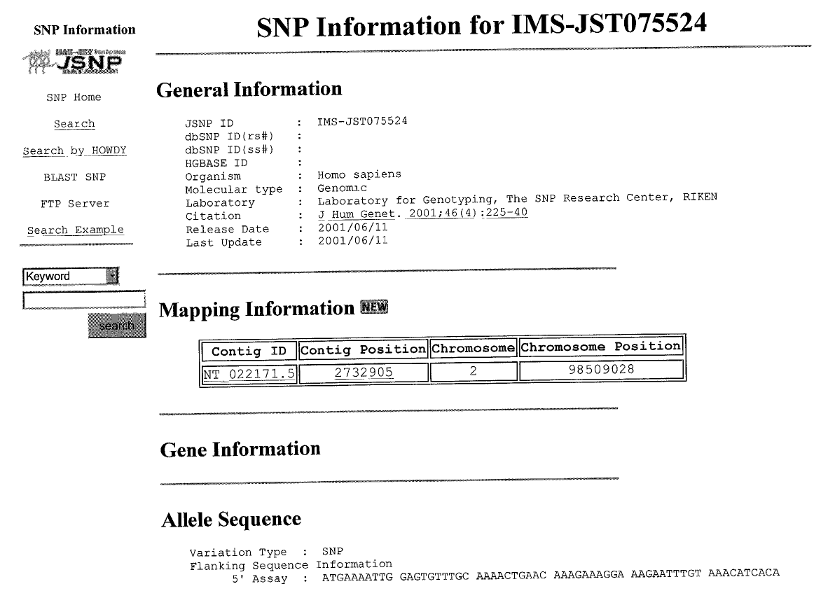

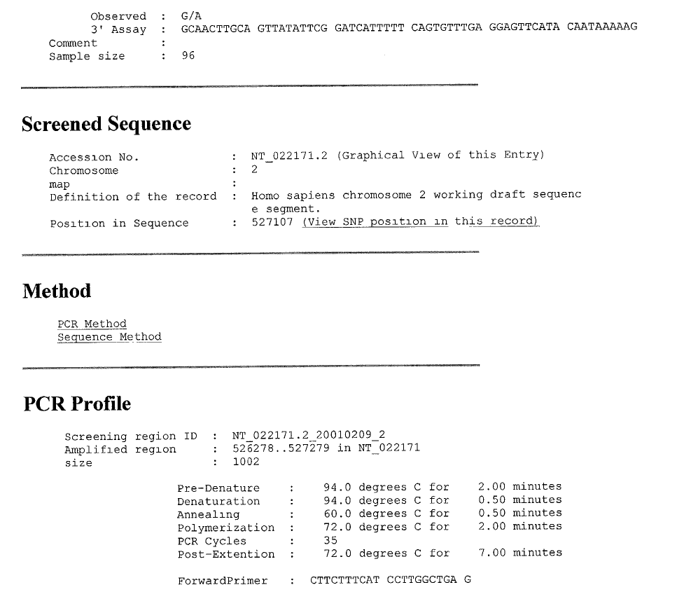

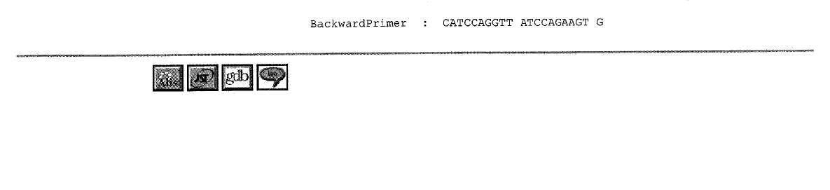

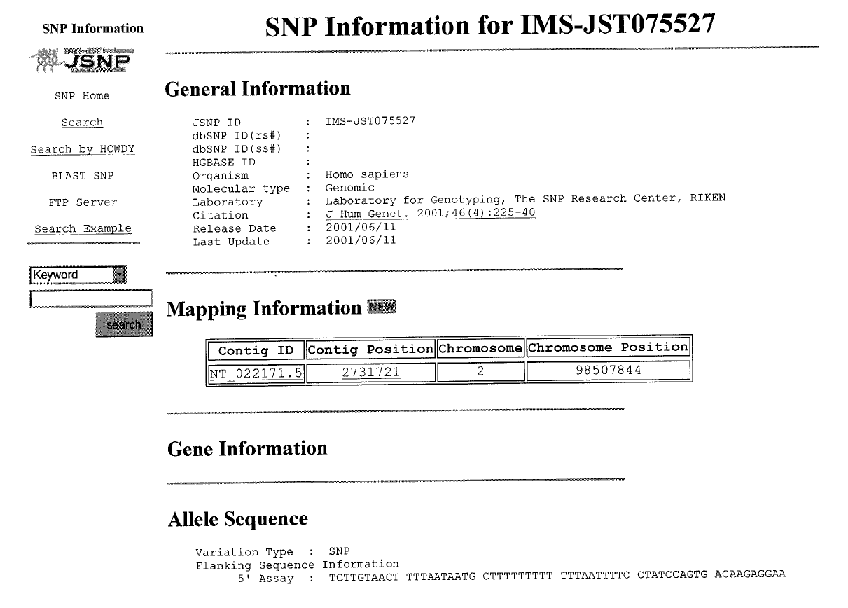

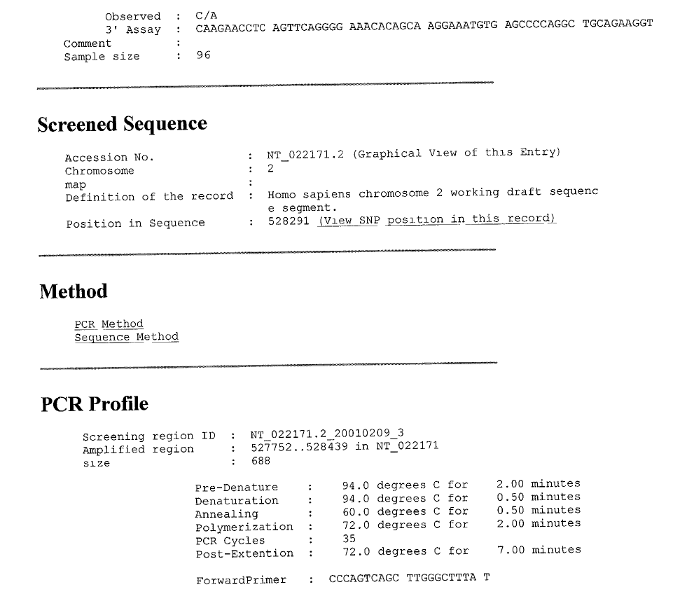

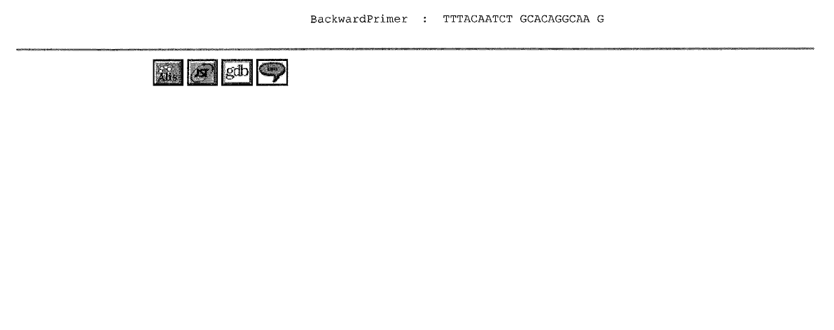

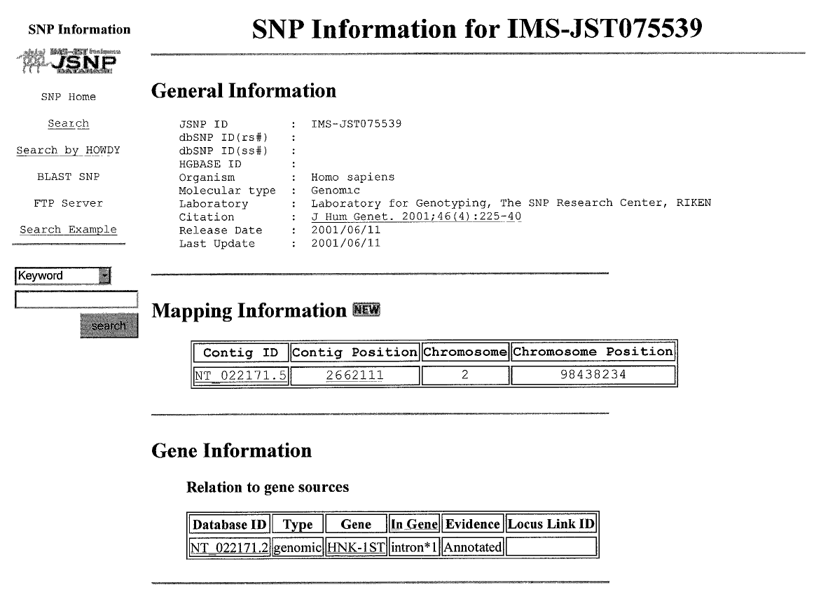

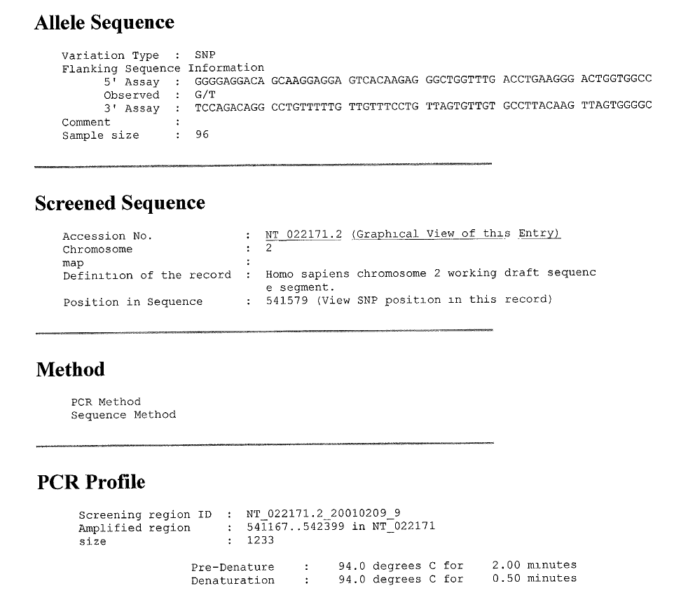

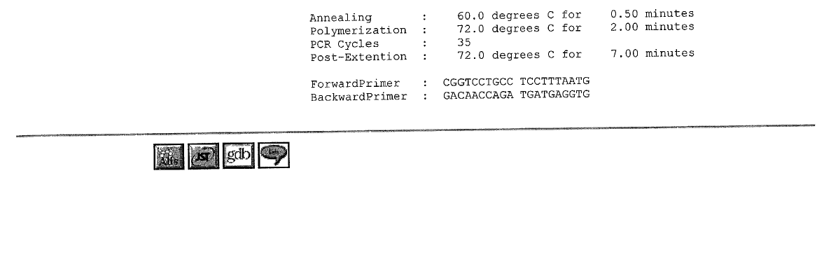

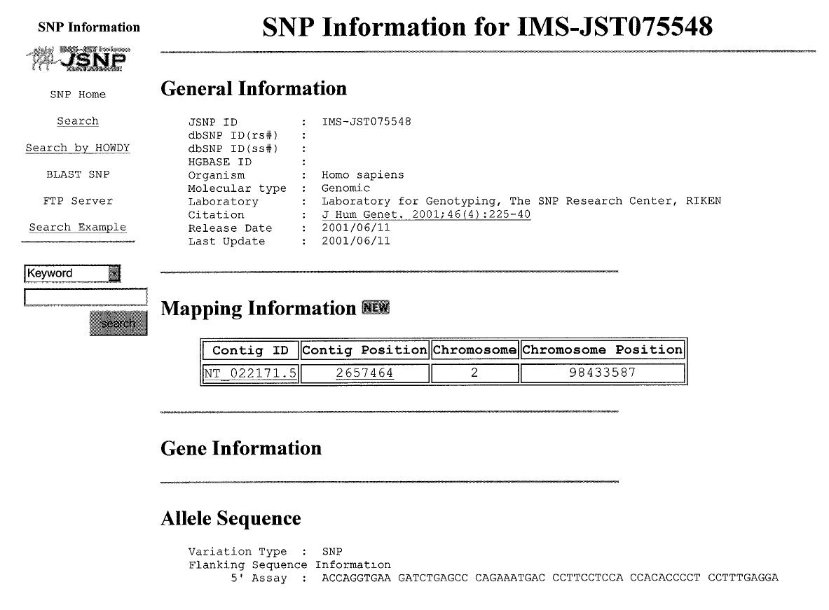

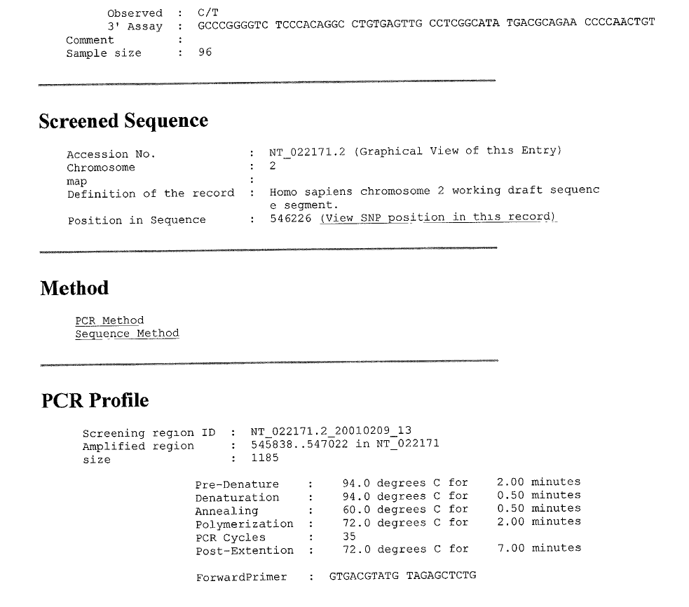

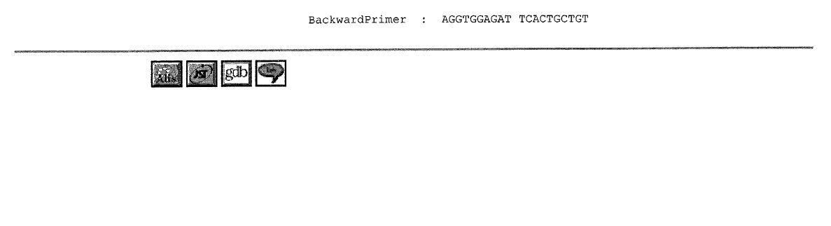

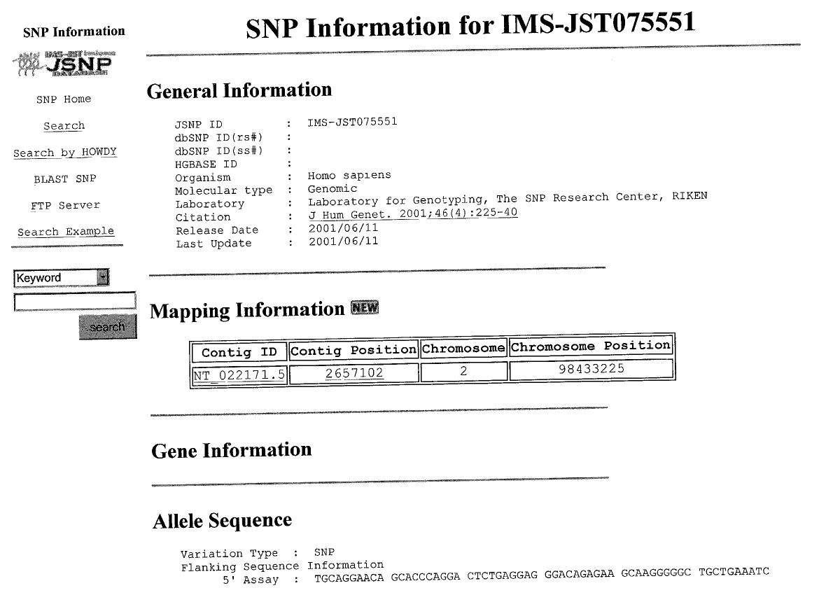

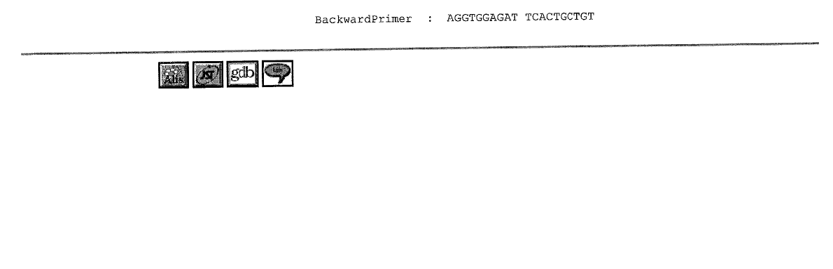

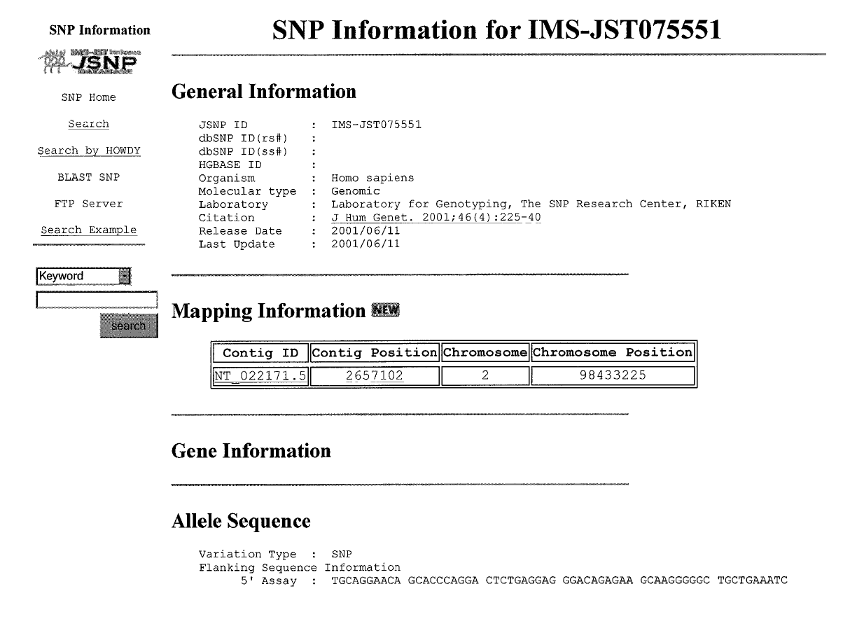

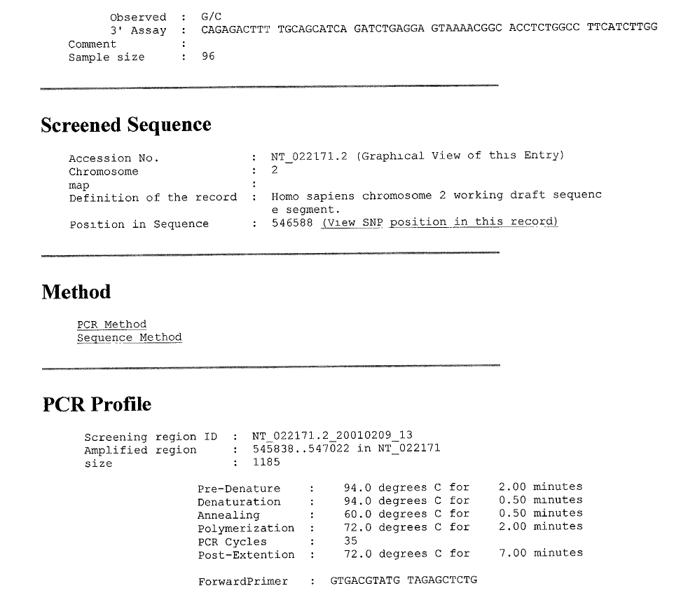

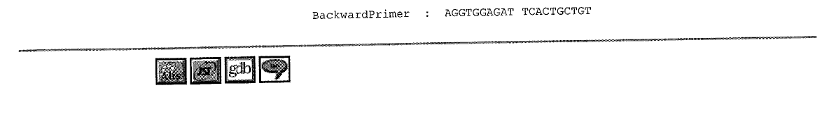

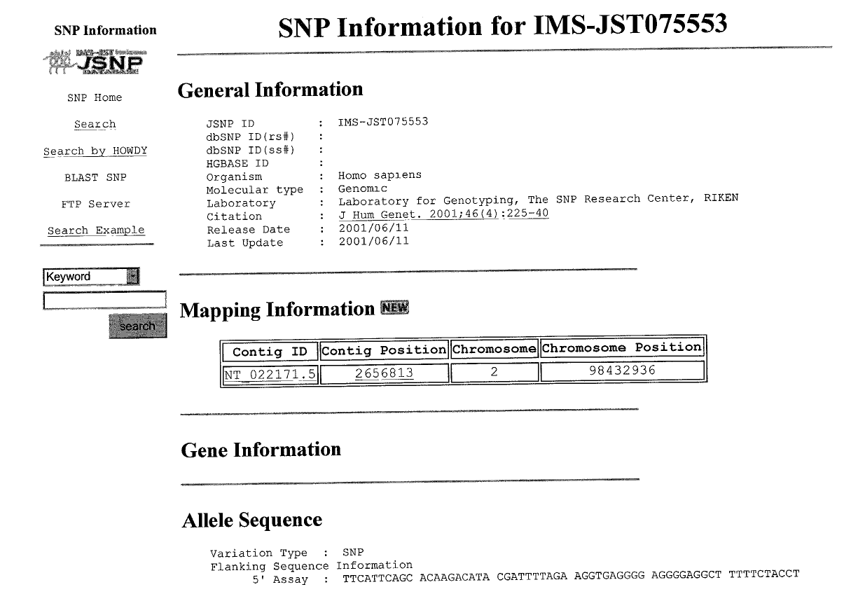

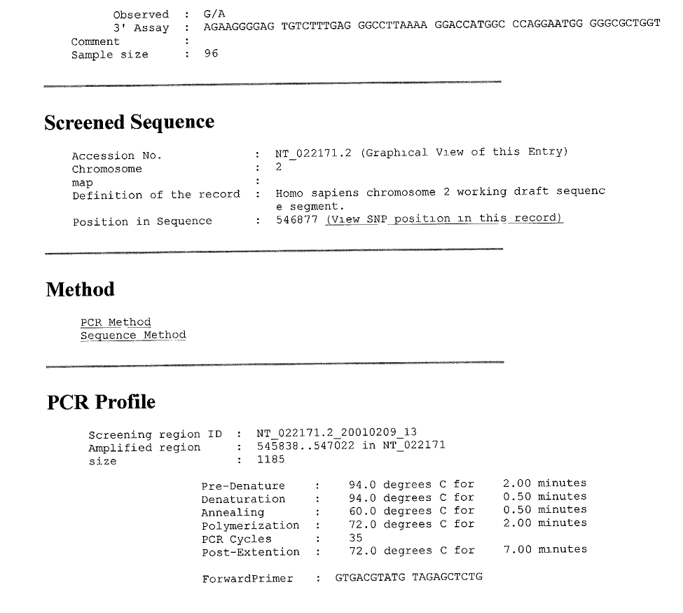

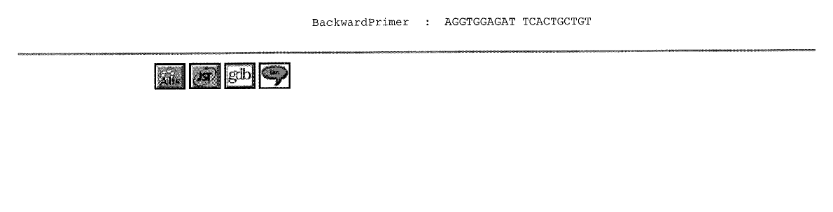

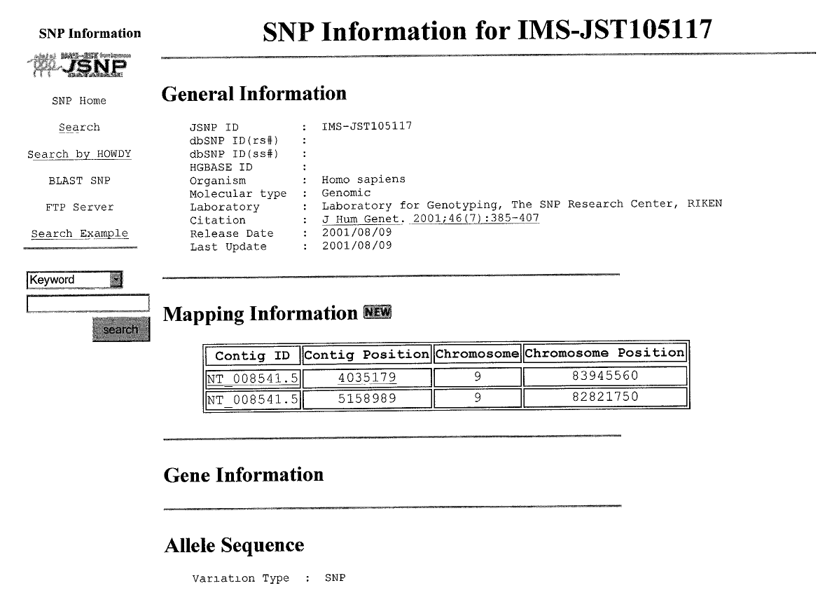

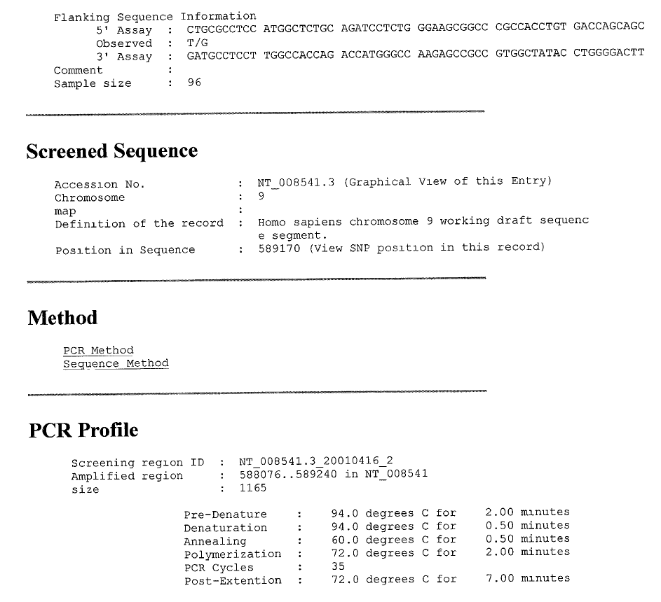

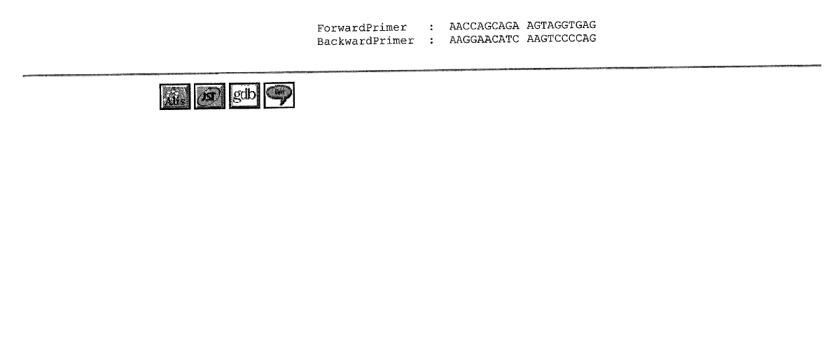

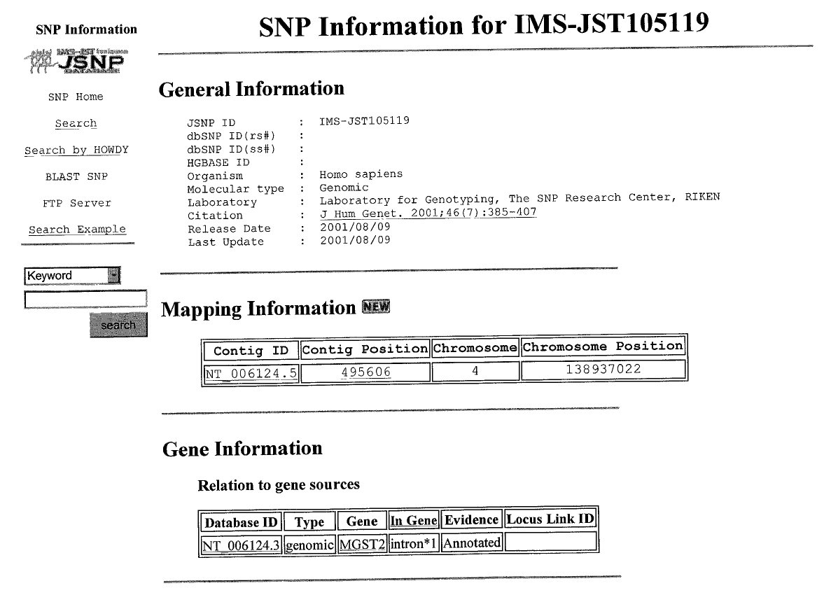

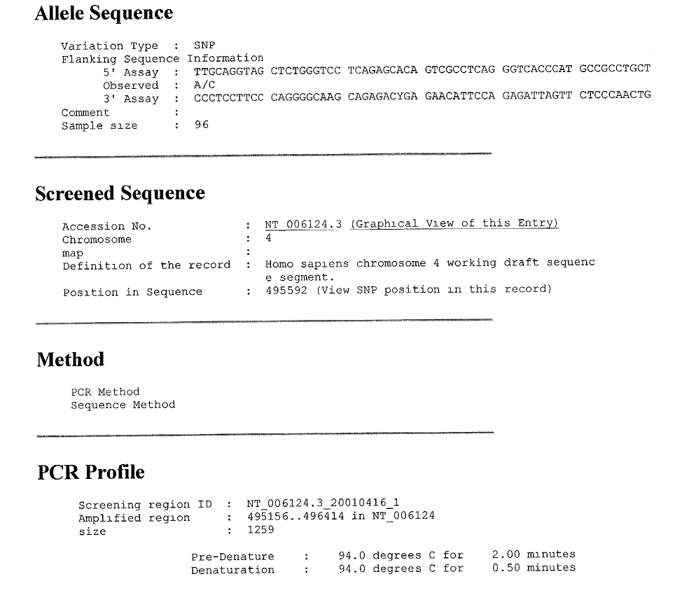

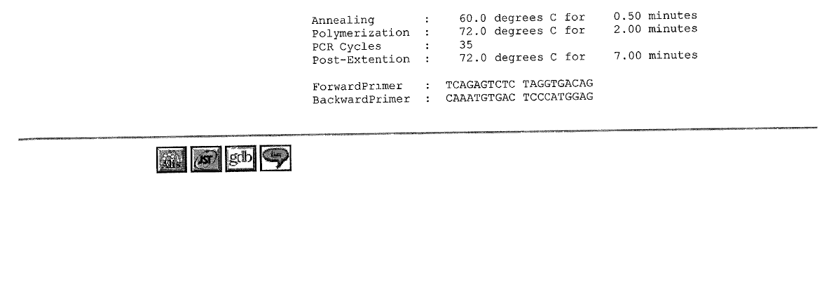

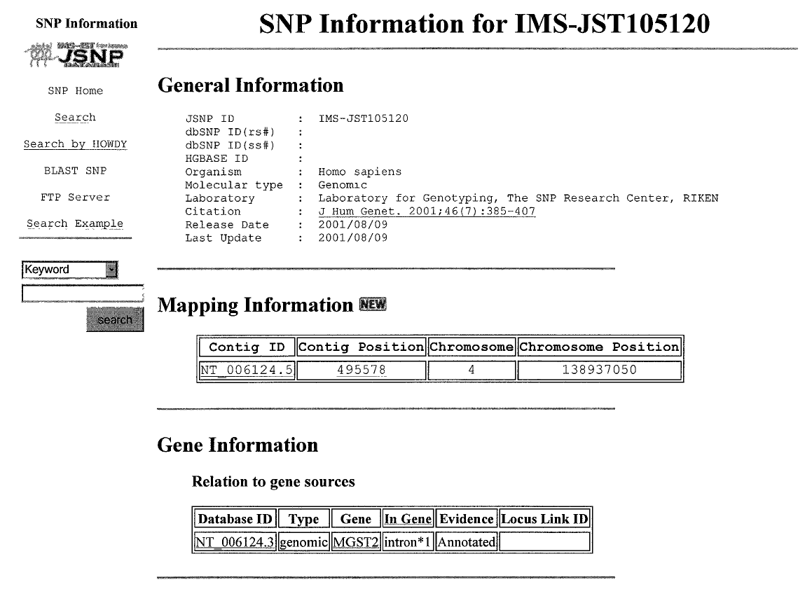

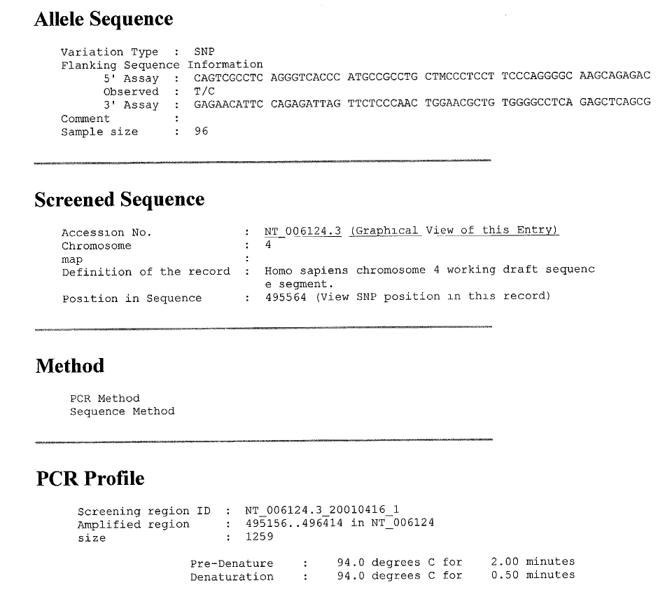

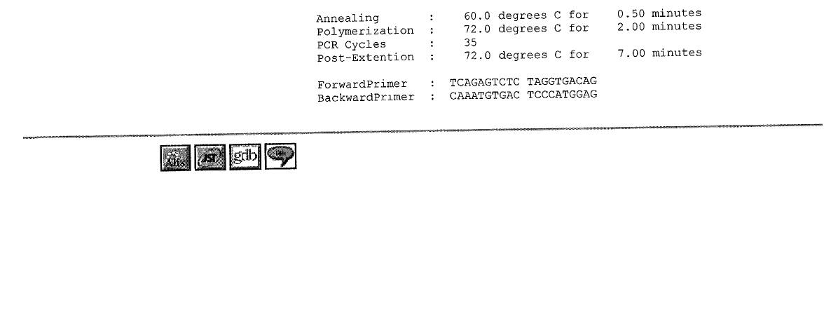

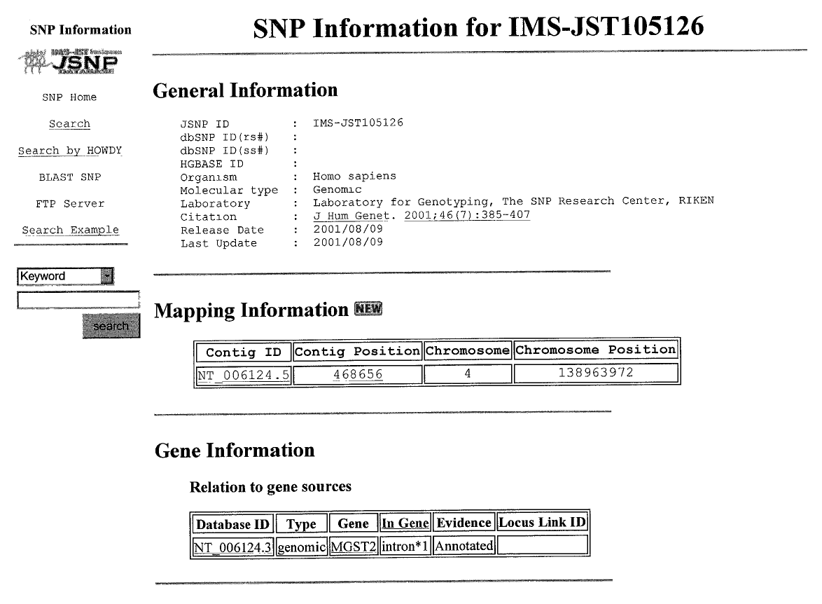

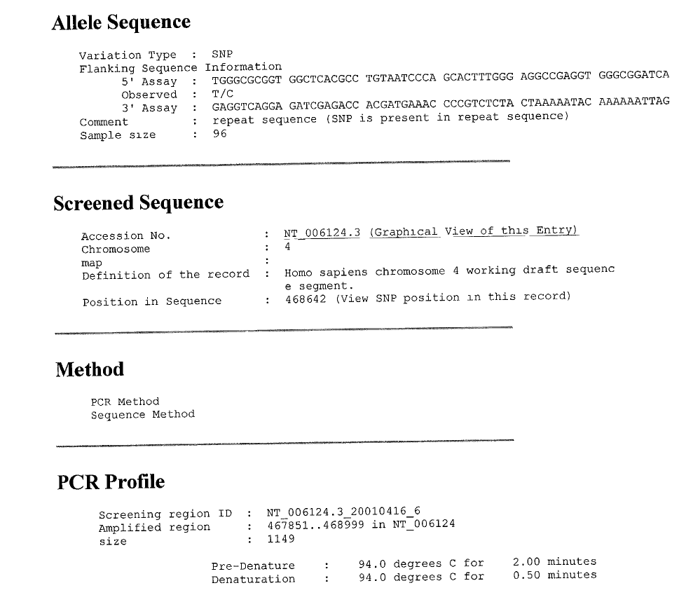

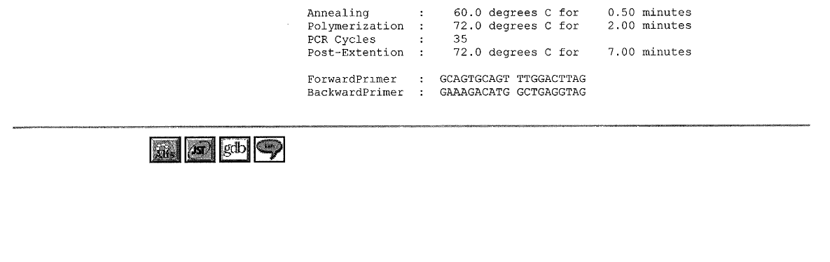

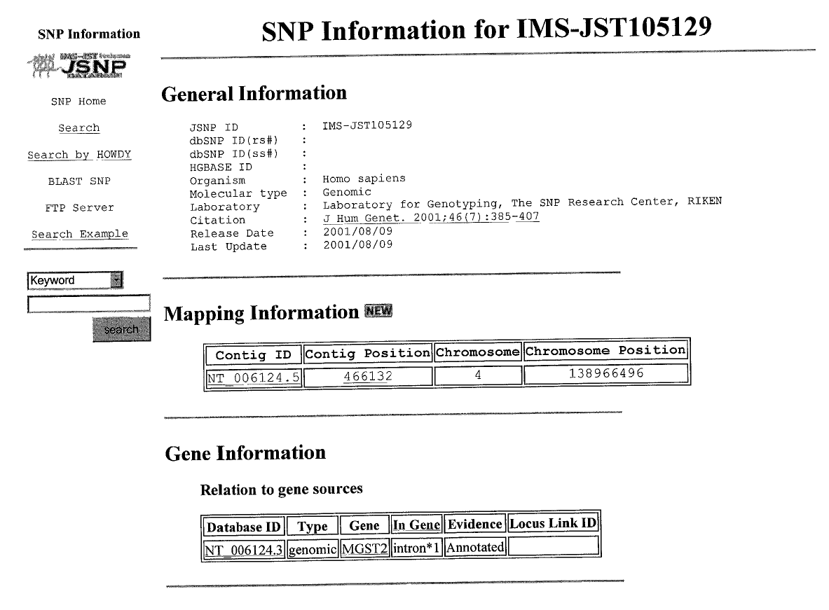

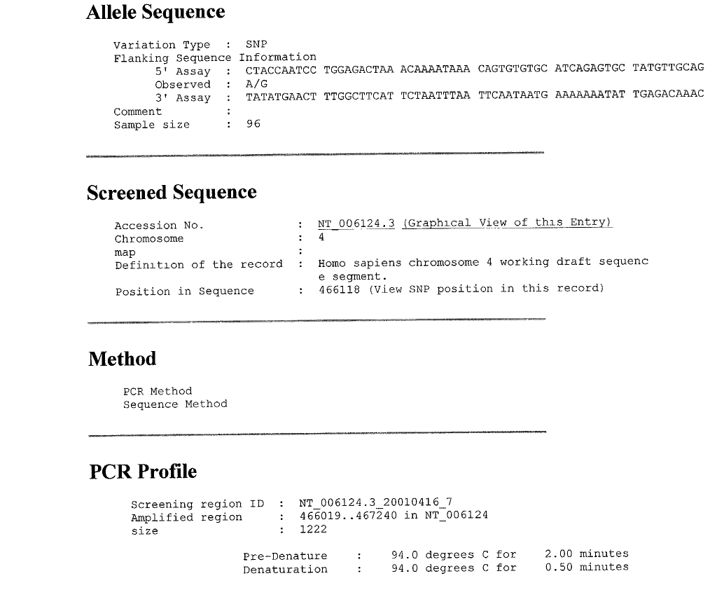

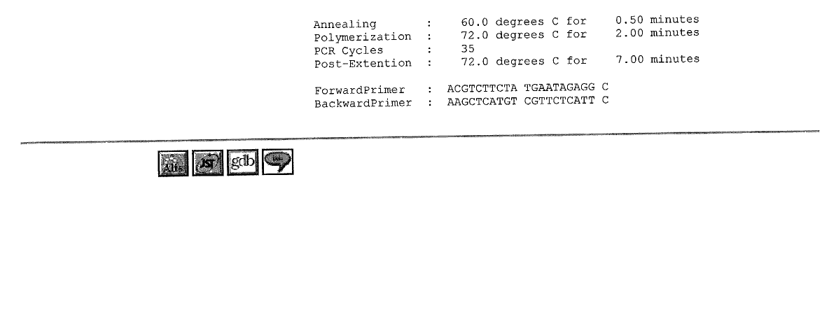

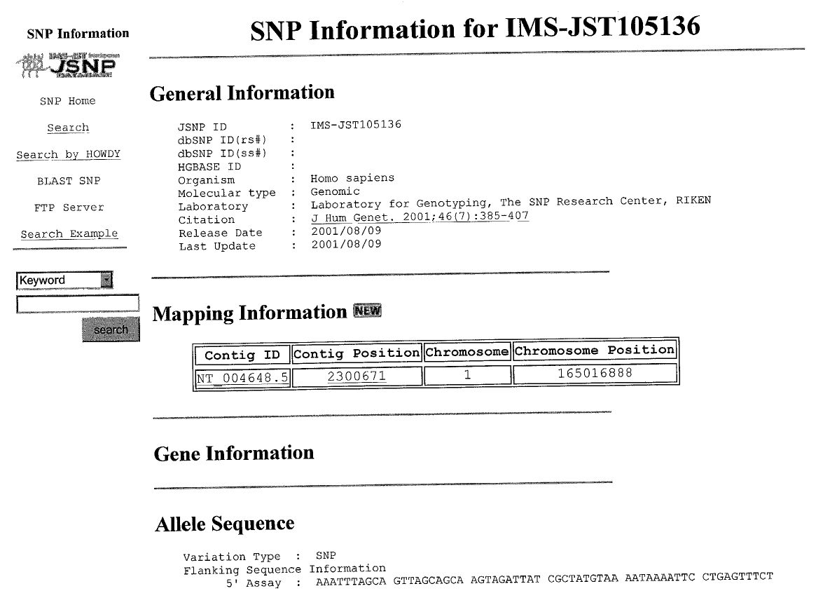

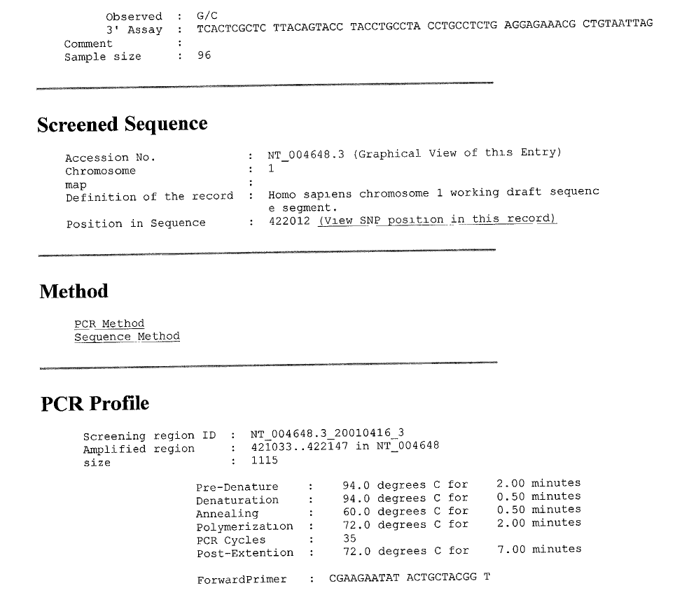

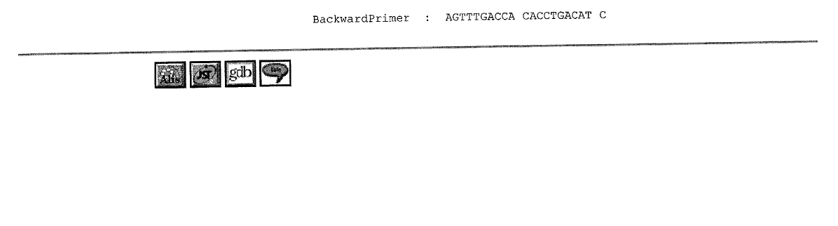

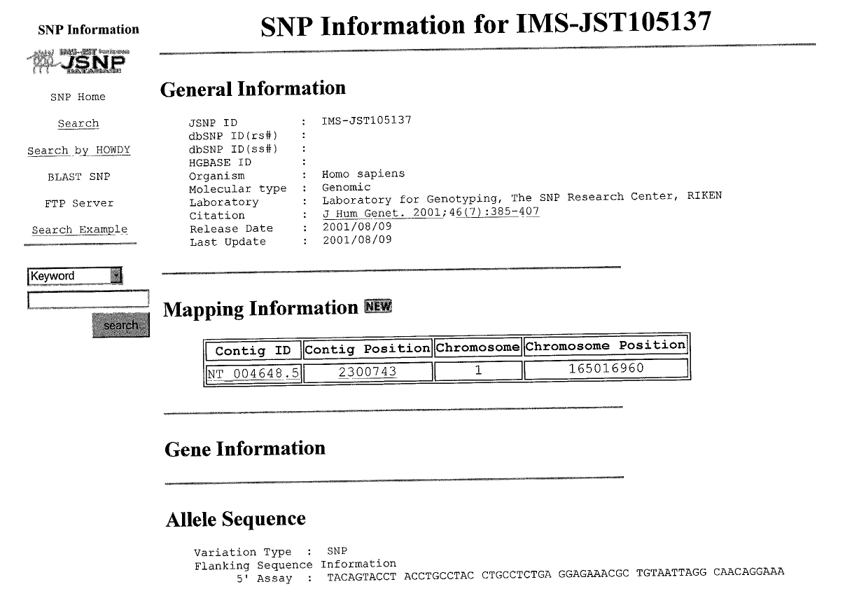

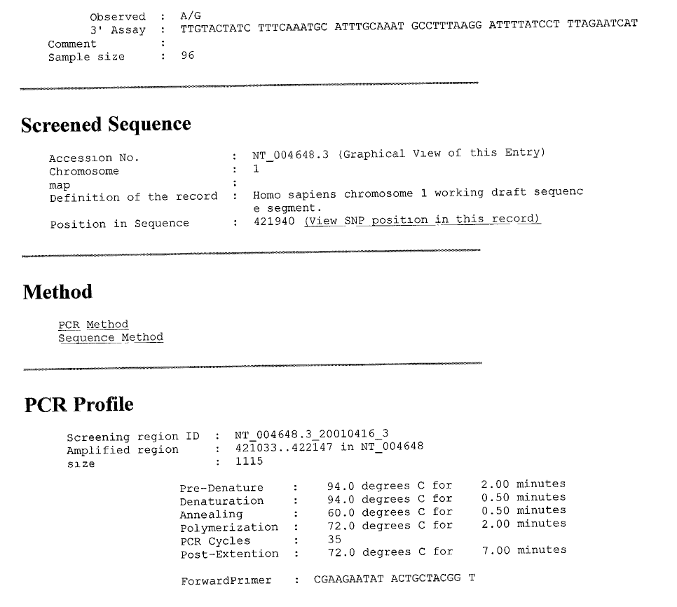

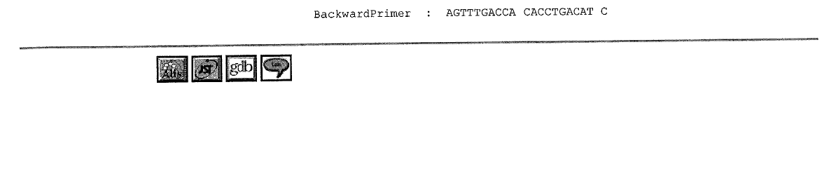

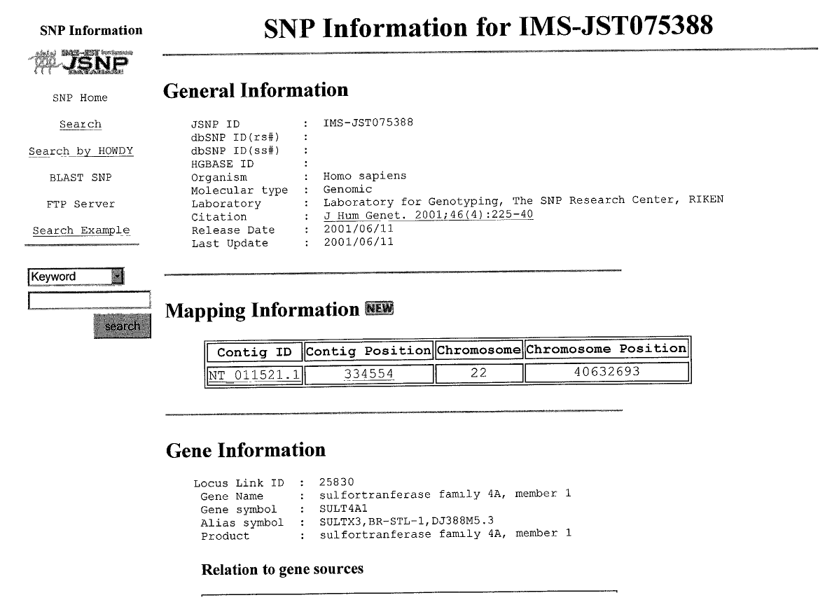

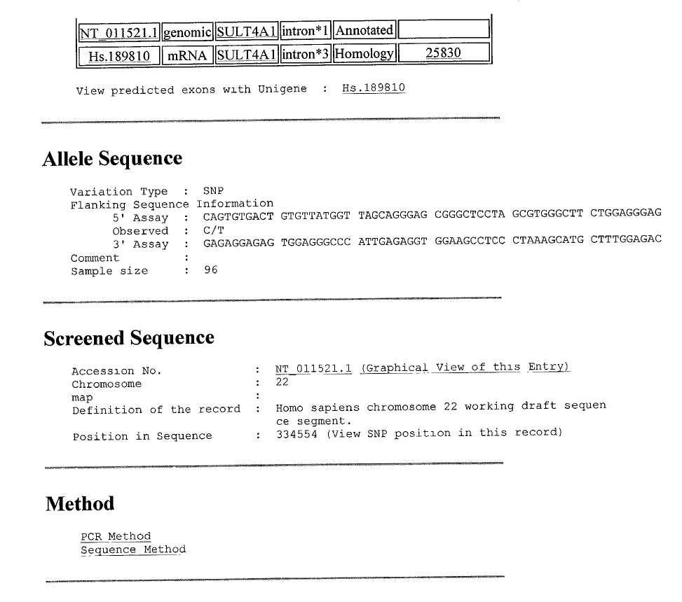

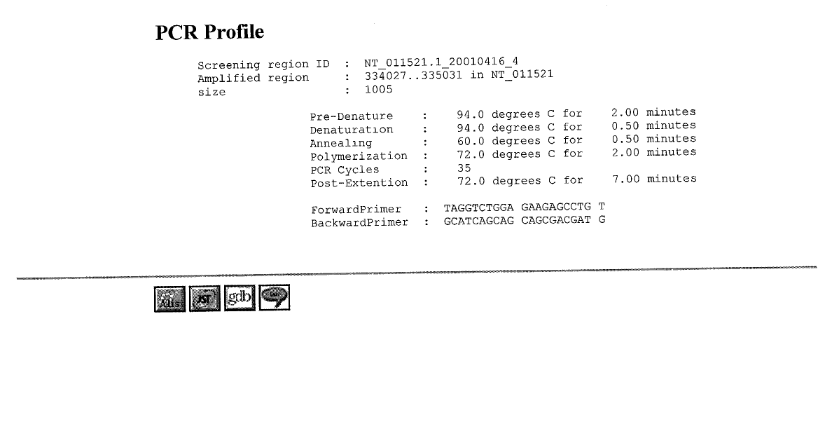

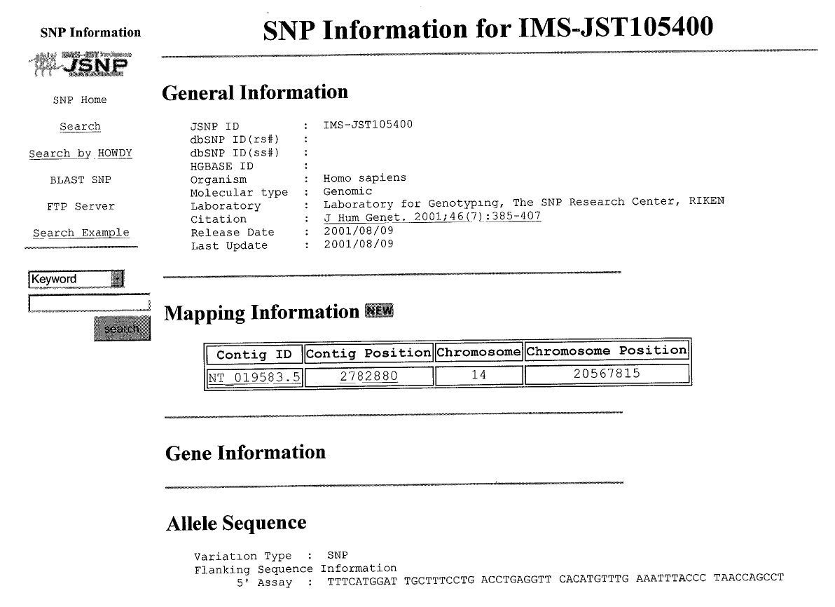

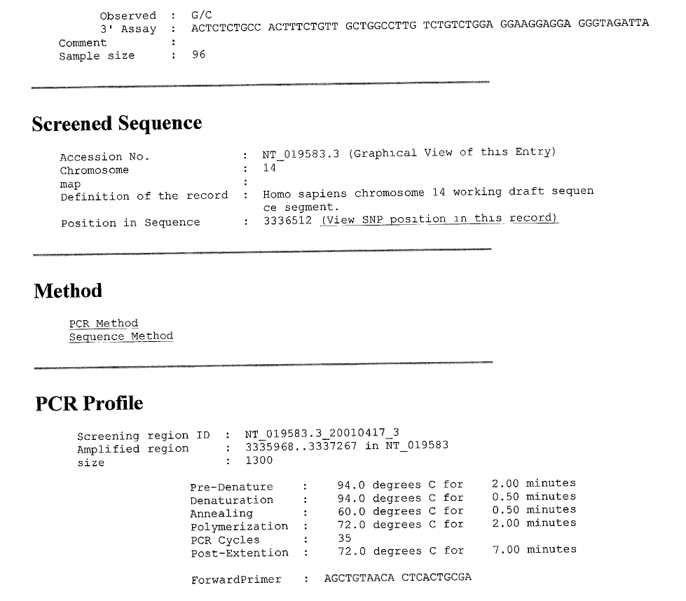

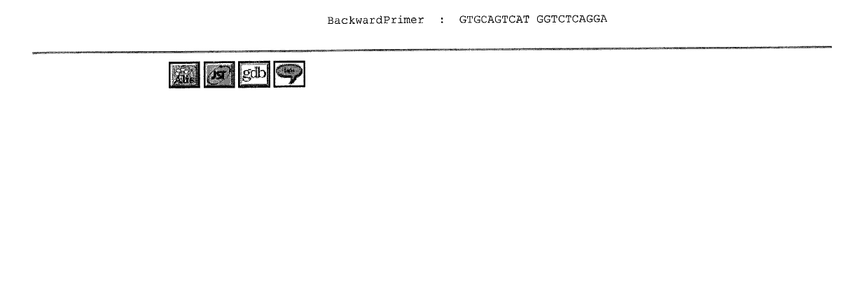

Here, “position” indicates the position of the SNP genome. The position of SNPs in the 5′ flanking region, intron region and 3′ flanking region are intron base sequences counted as a single number starting at the exon-intron junction. The position of SNPs in the exon region are exon base sequences counted as a single number starting at the exon-intron junction. Also, (+) or no symbol indicates a number counted in the 3′ upstream direction and (−) indicates a number counted in the 5′ downstream direction. The number in the “number” column indicates the position of the SNP in the gene maps of the various genes (FIG. 9 through FIG. 141 and FIG. 144 through 312).

The sequence represented by the SEQ ID Nos. 1-3360 and 3361-7669 can readily be associated with the corresponding gene, chromosome, and chromosomal position. Each of the genes shown in Table 1 correlates to a corresponding Figure in the present application. The Figures show a map of the gene with positional identifiers for each of the polymorphisms. The Figures also provide an accession number that correlates to public genome databases, allowing the genetic context of the polymorphism and the gene to be understood. Using the information in Table 1, the Figures, and public genome databases, one skilled in the art is able to identify flanking sequences. This allows, for example, the development of PCR primers that flank the polymorphism. Considerations for PCR primer design are known in the art for both single PCR reactions and multiplex reactions (See e.g., Henegariu et al., BioTechniques 23:504-511 [1997] and PCR Applications, edited by Innis, Gelfand, and Sninsky, Academic Press, San Diego, Calif. 1999), each of which is herein incorporated by references in its entirety). Examples of primers that find use in the amplification of sequences containing polymorphisms, as well as amplification conditions, are found at the IMS-JST JSNP database website (See, submissions from Laboratory for Genotyping, The SNP Research Center, The Institute of Physical and Chemical Research (RIKEN)).

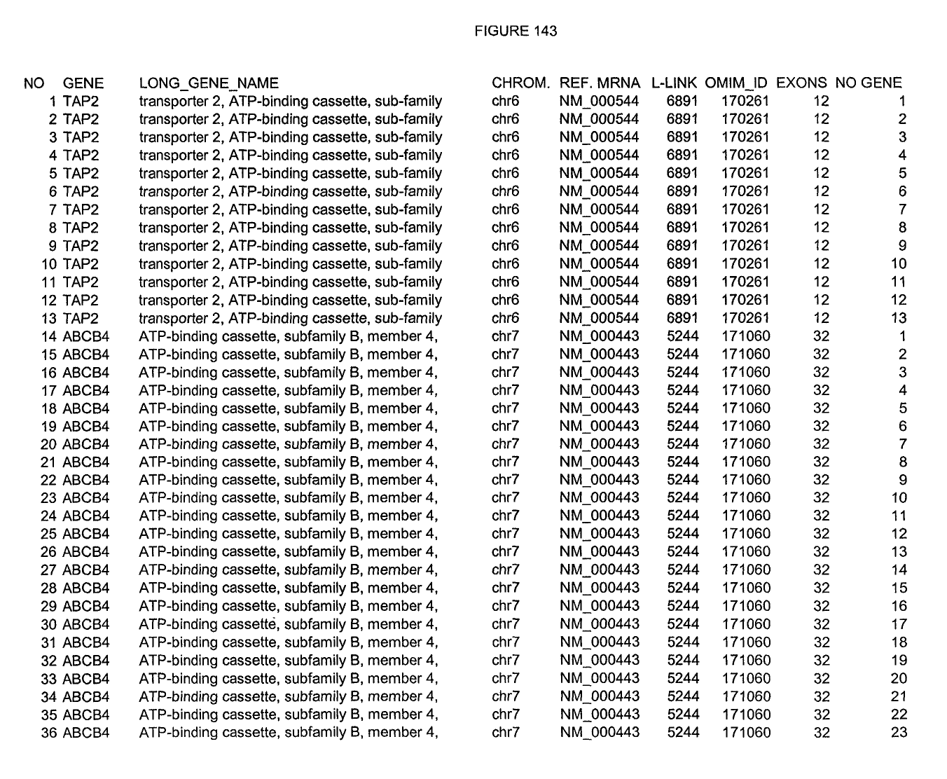

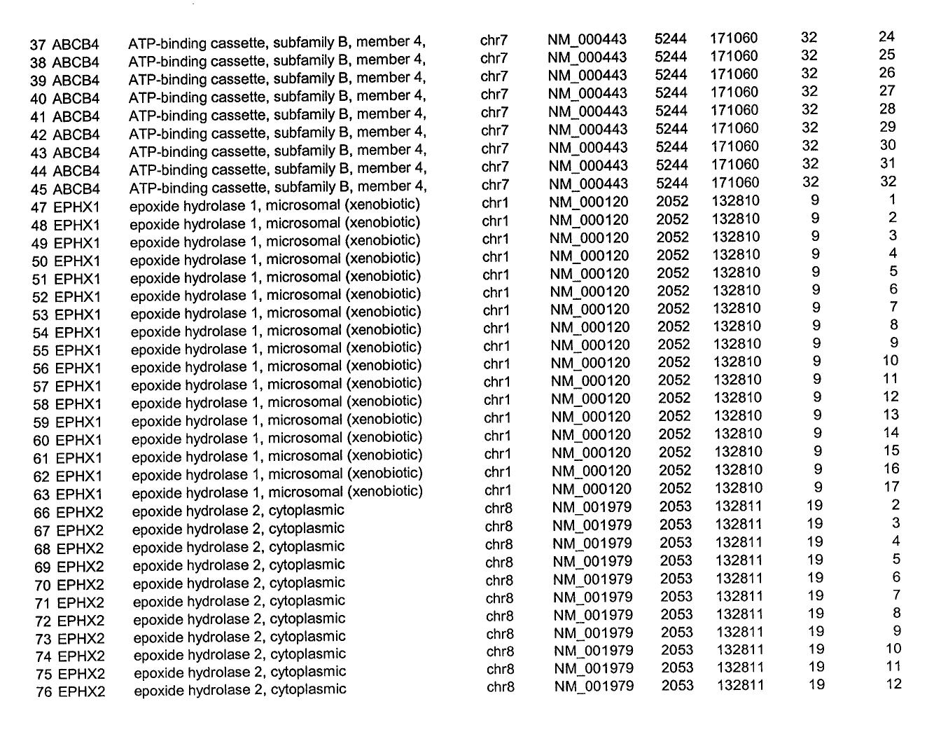

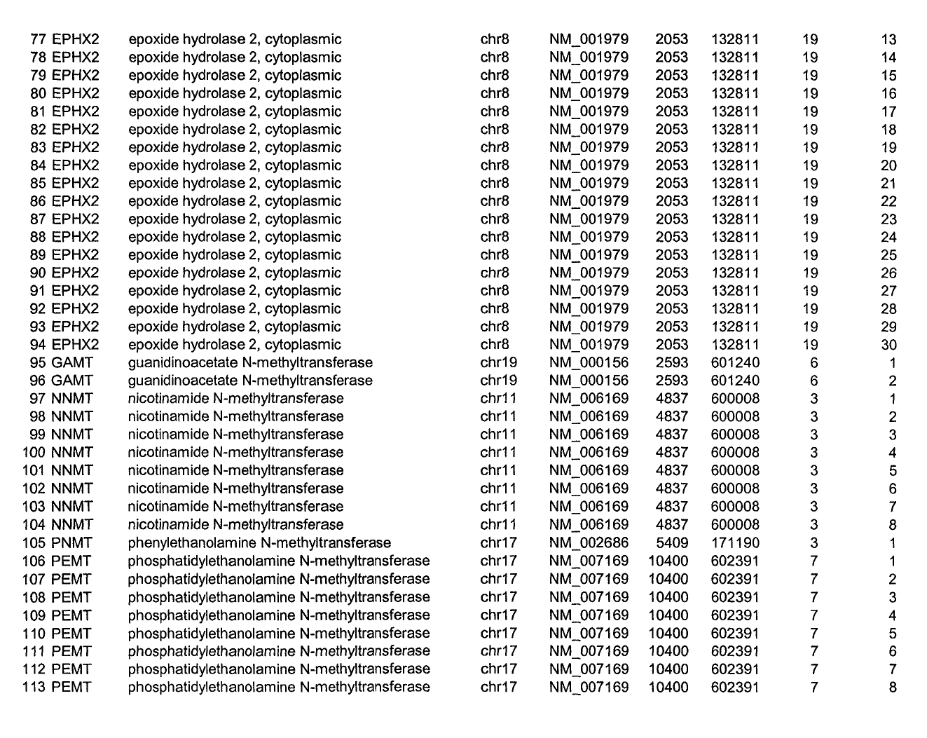

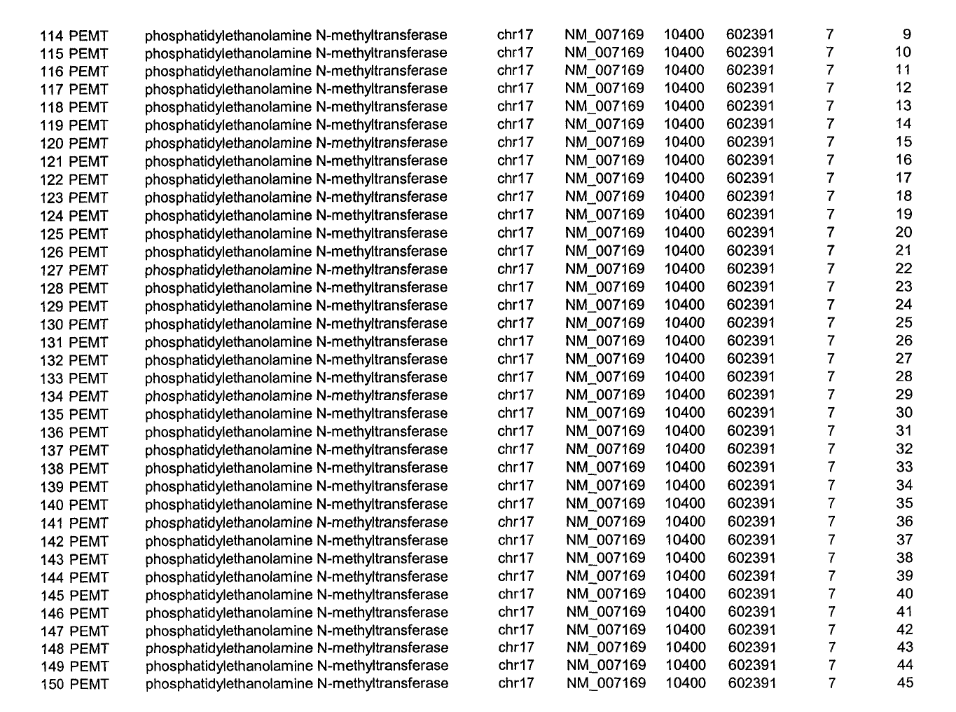

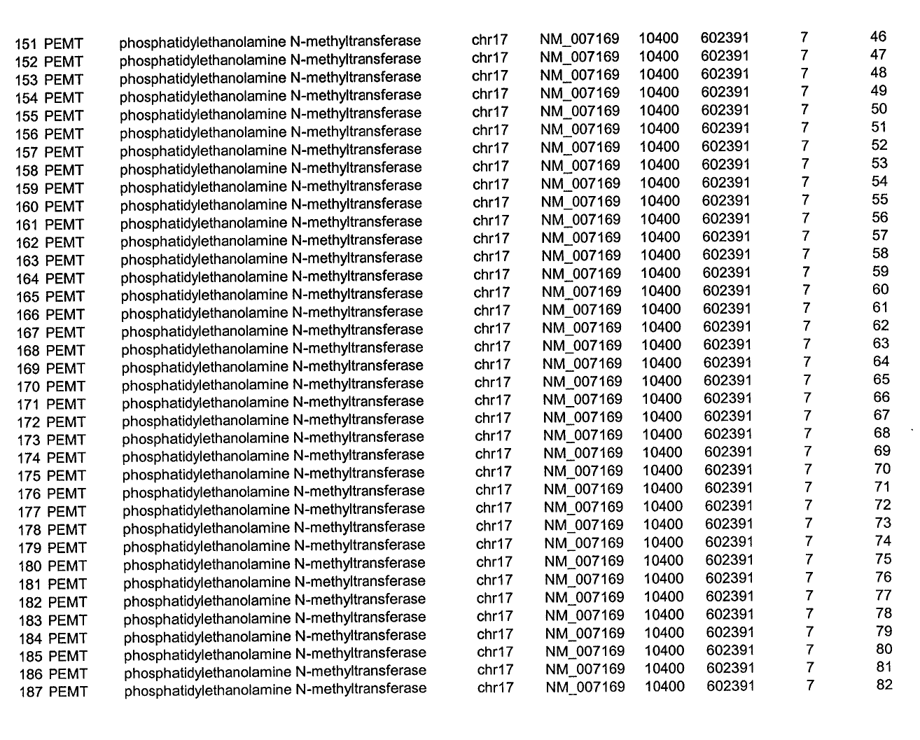

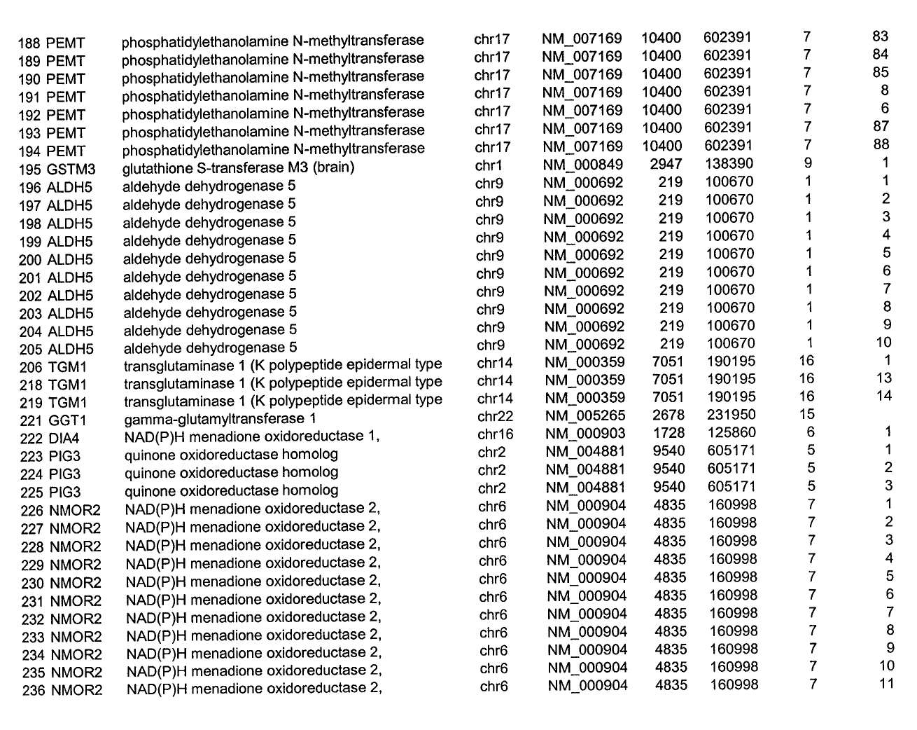

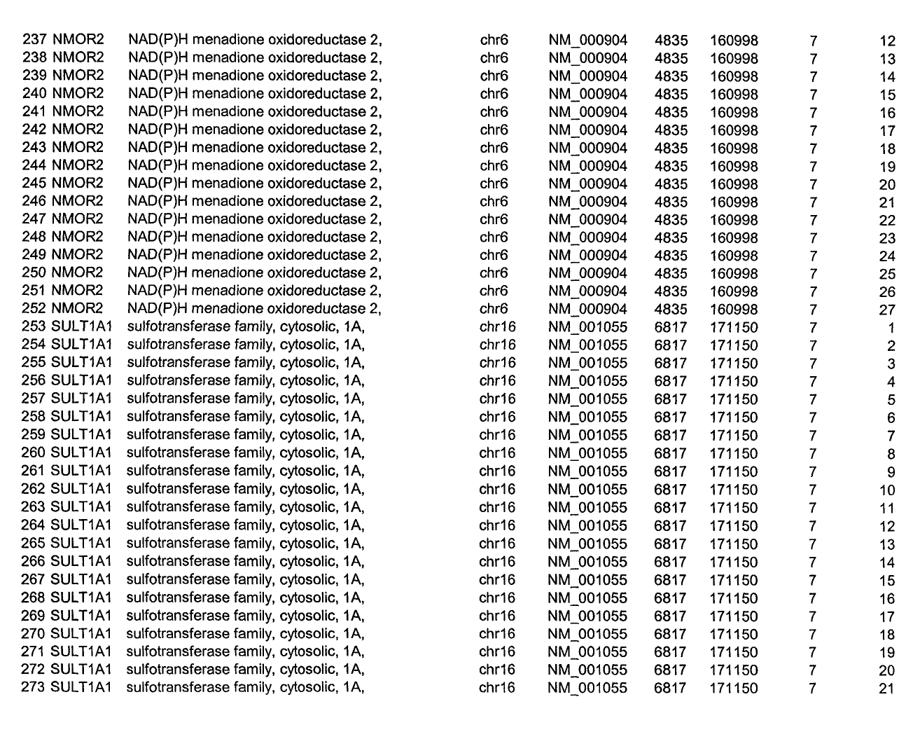

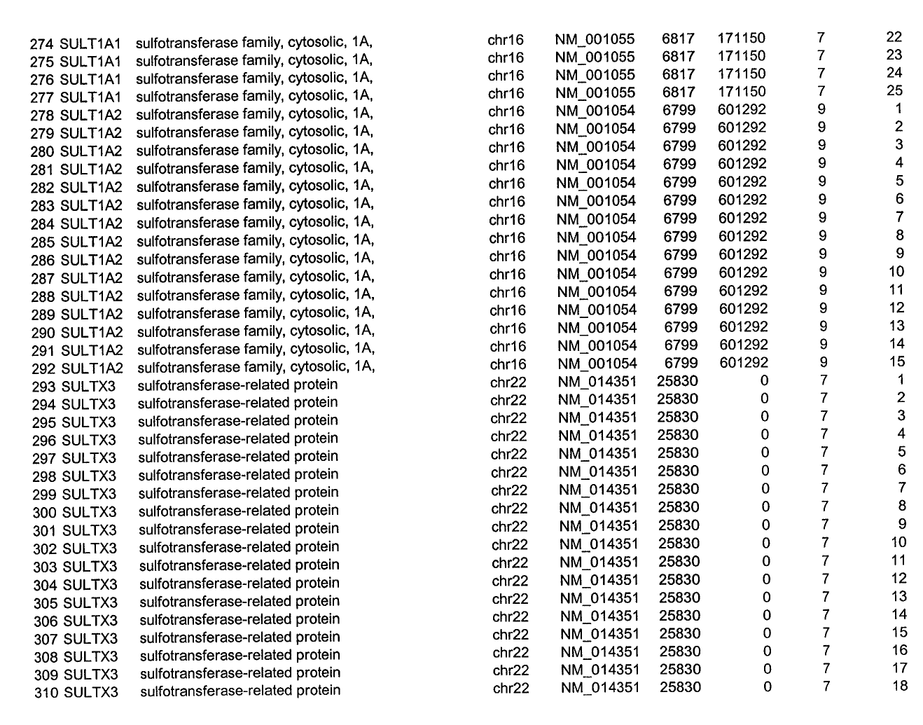

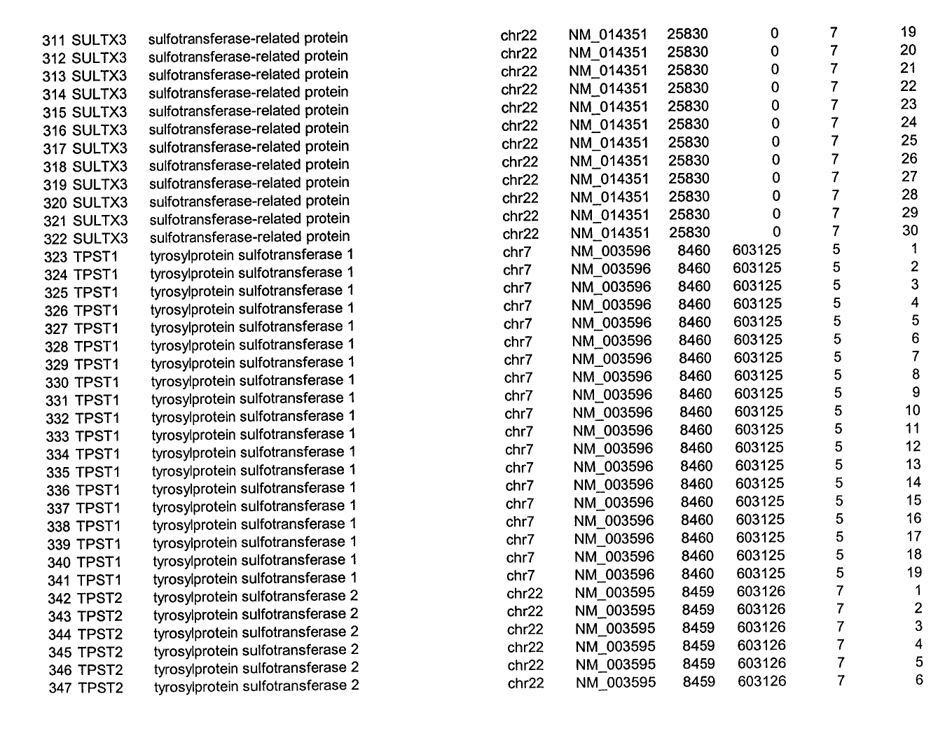

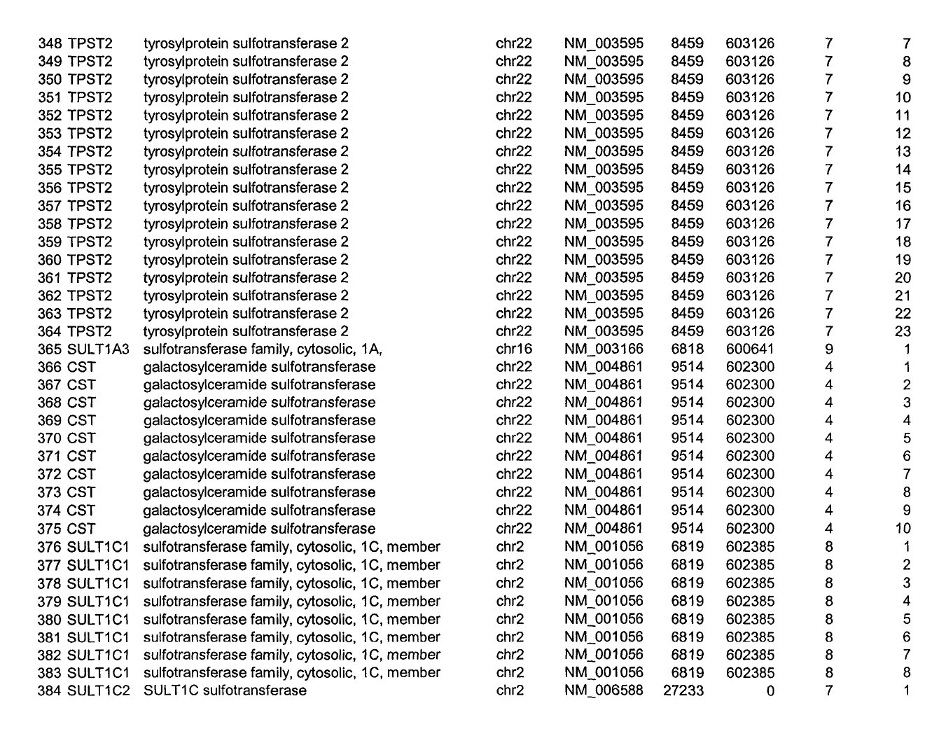

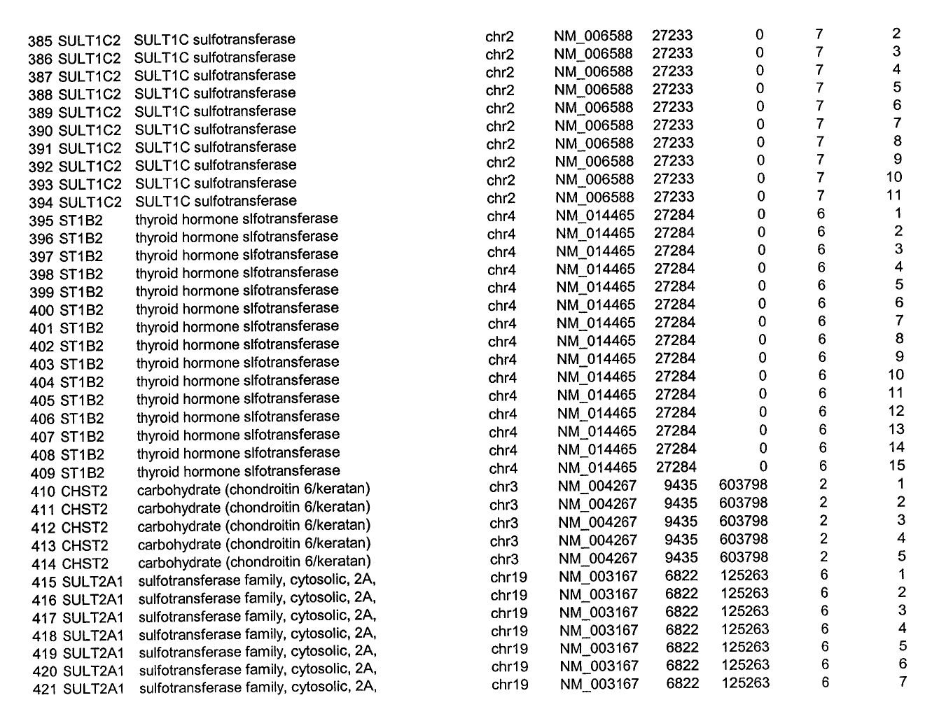

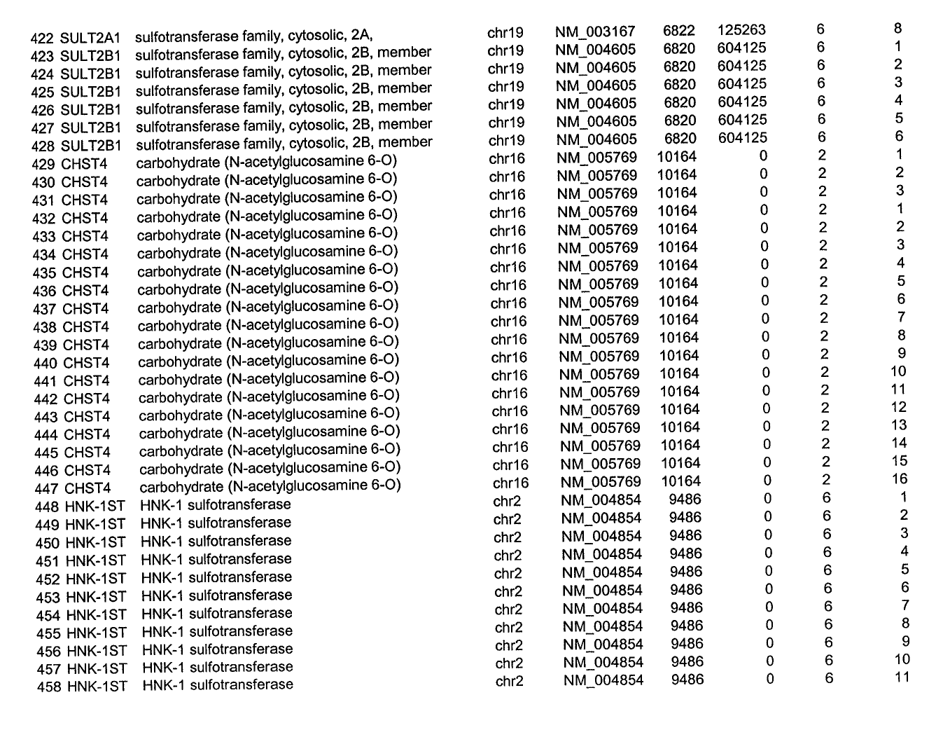

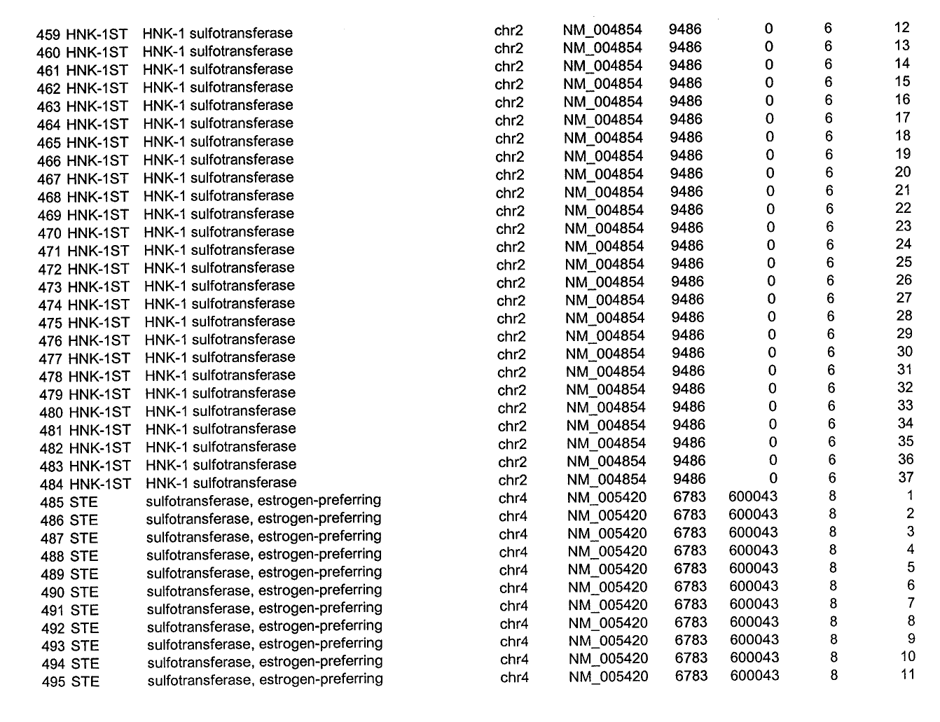

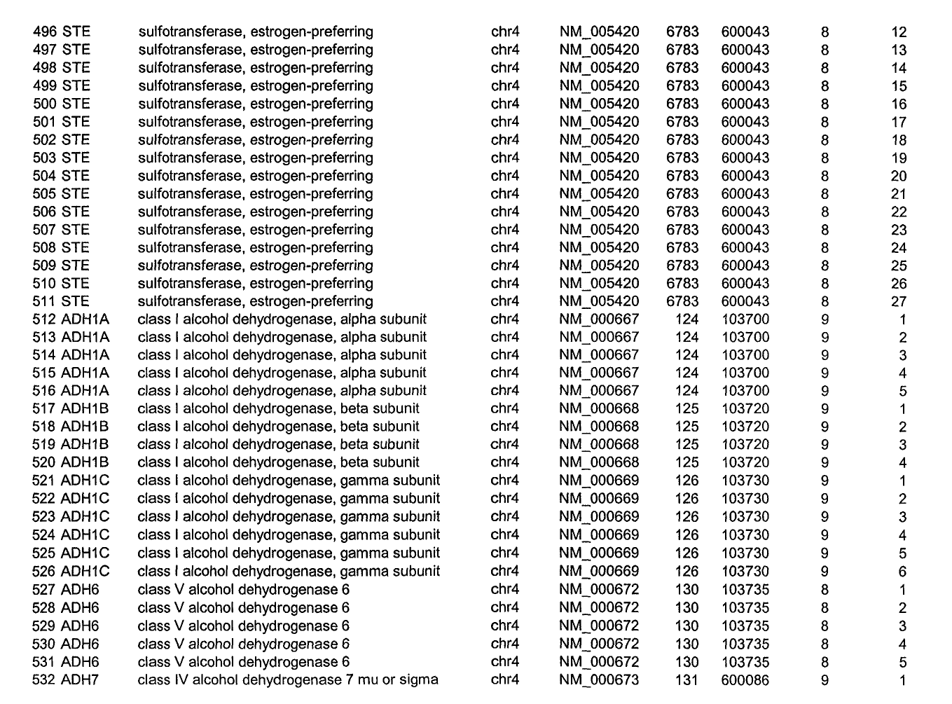

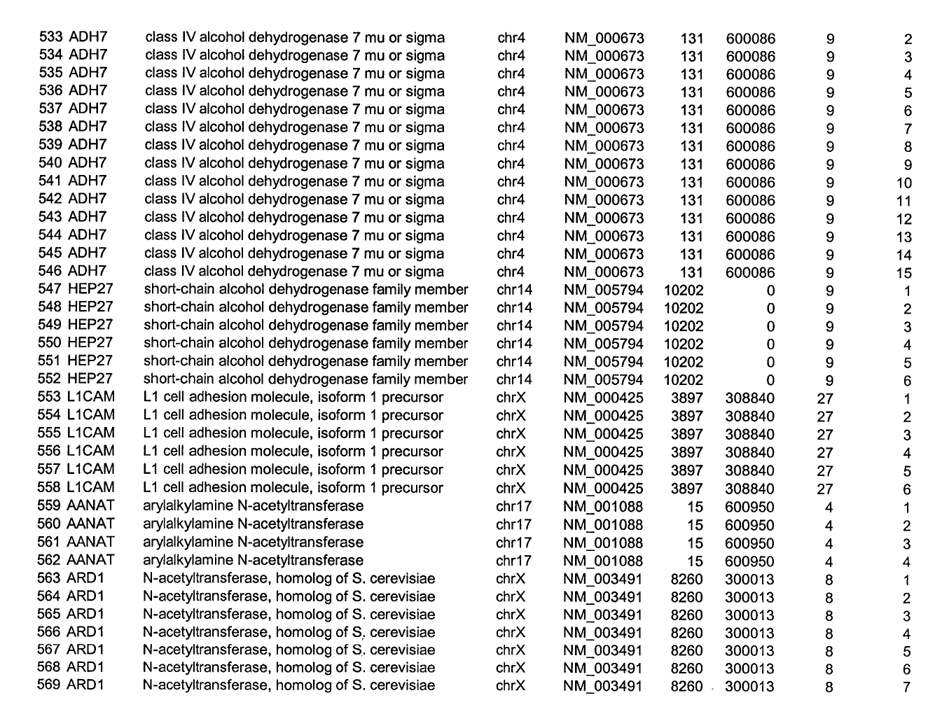

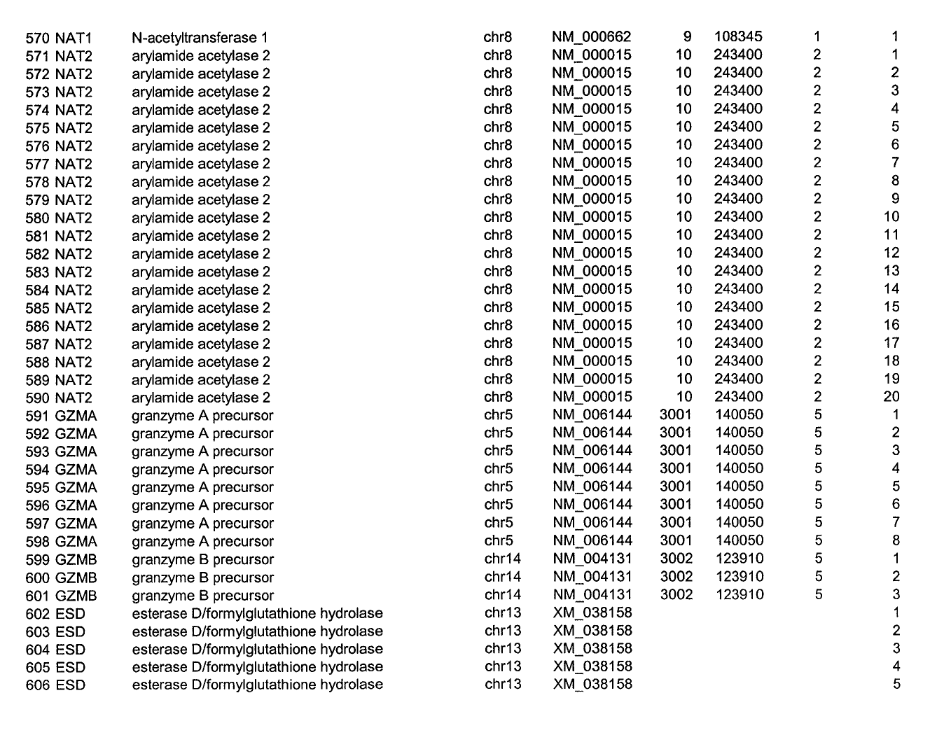

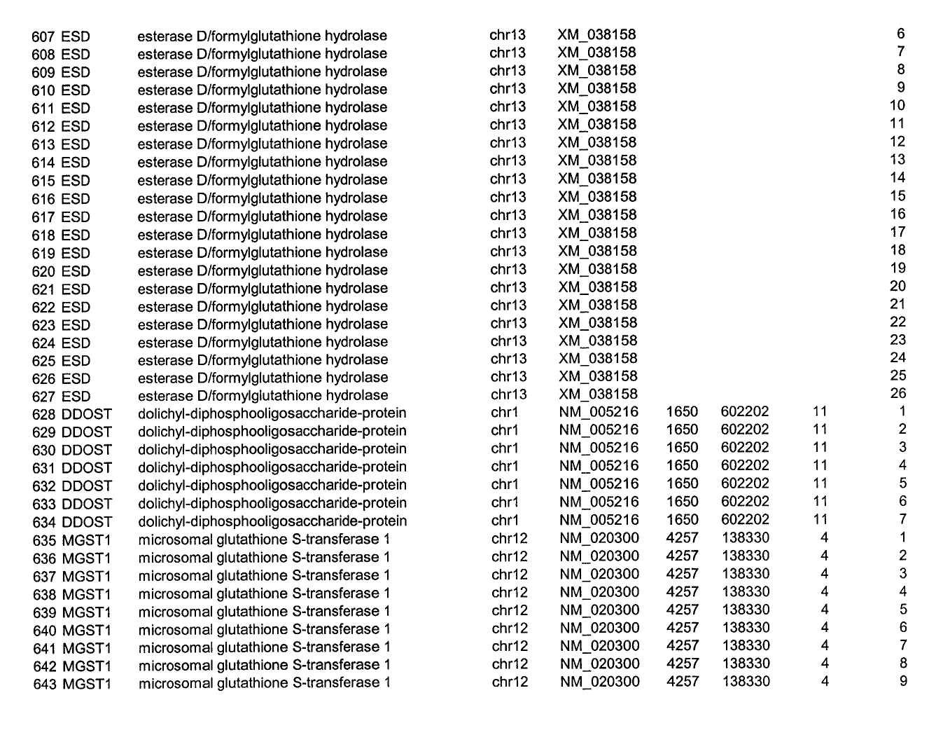

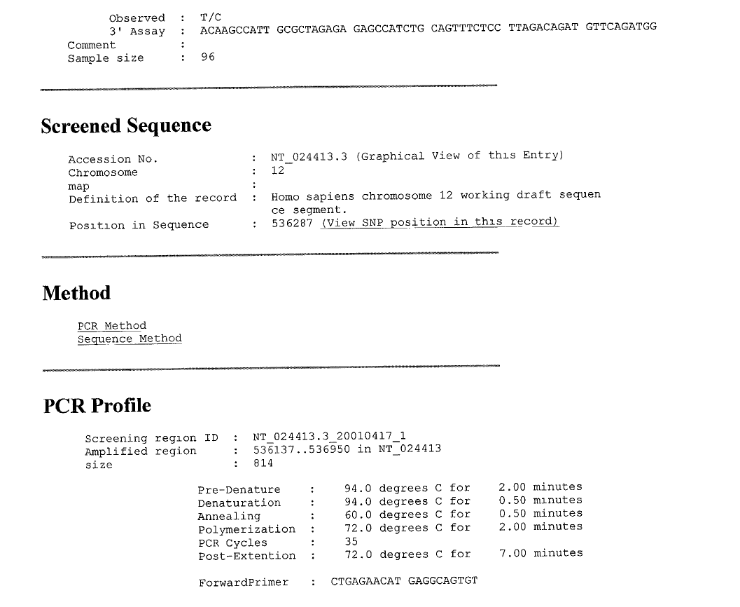

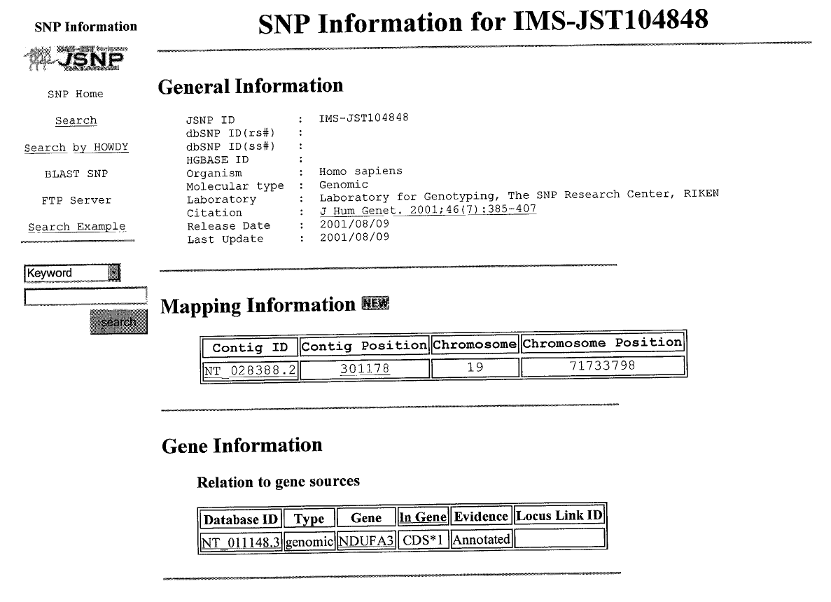

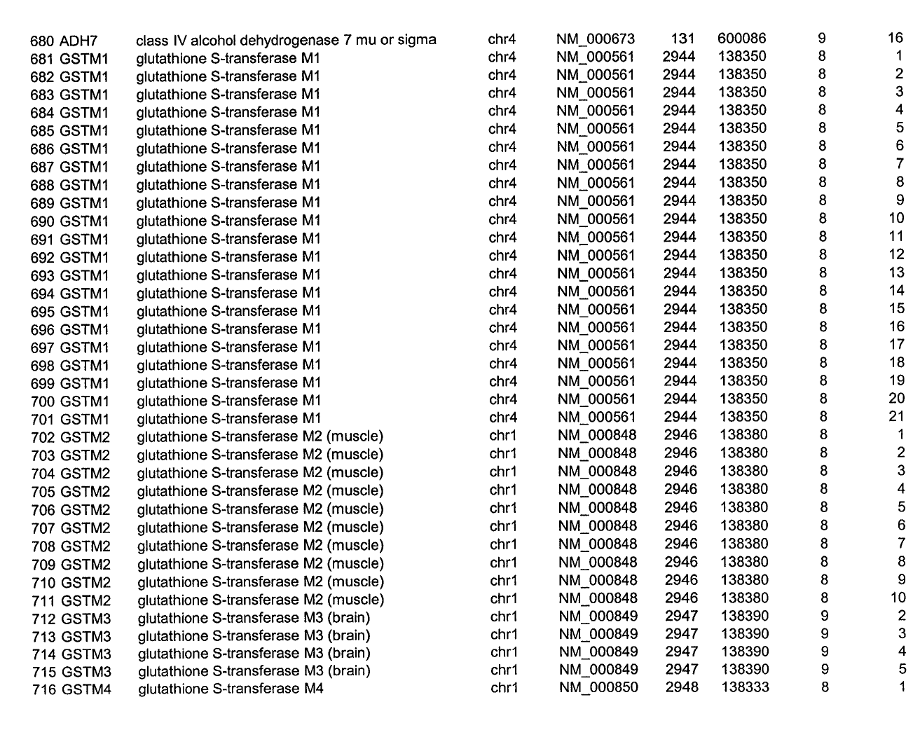

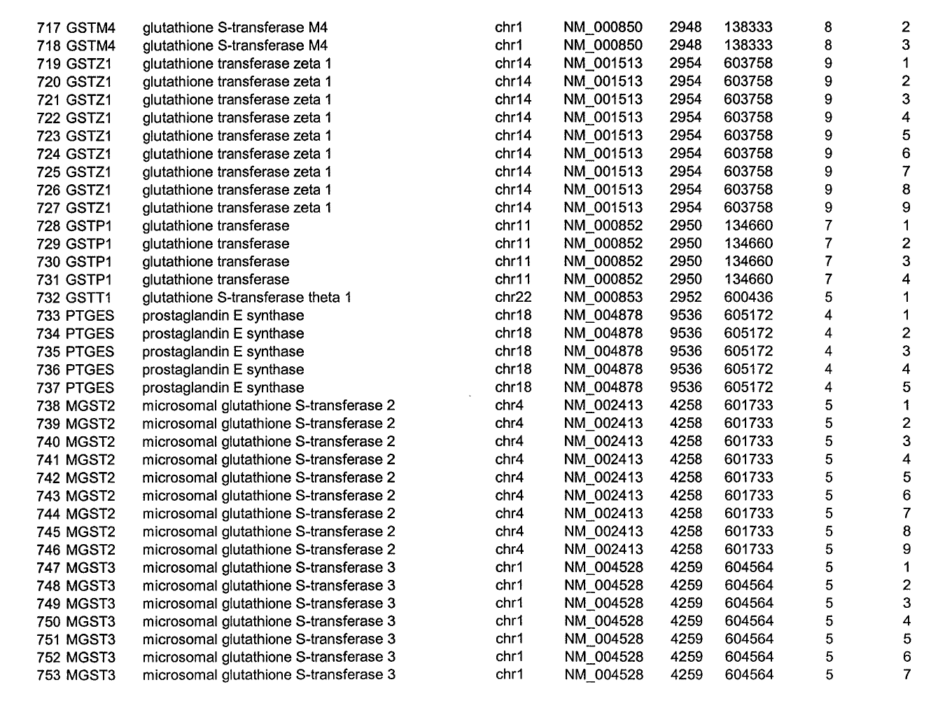

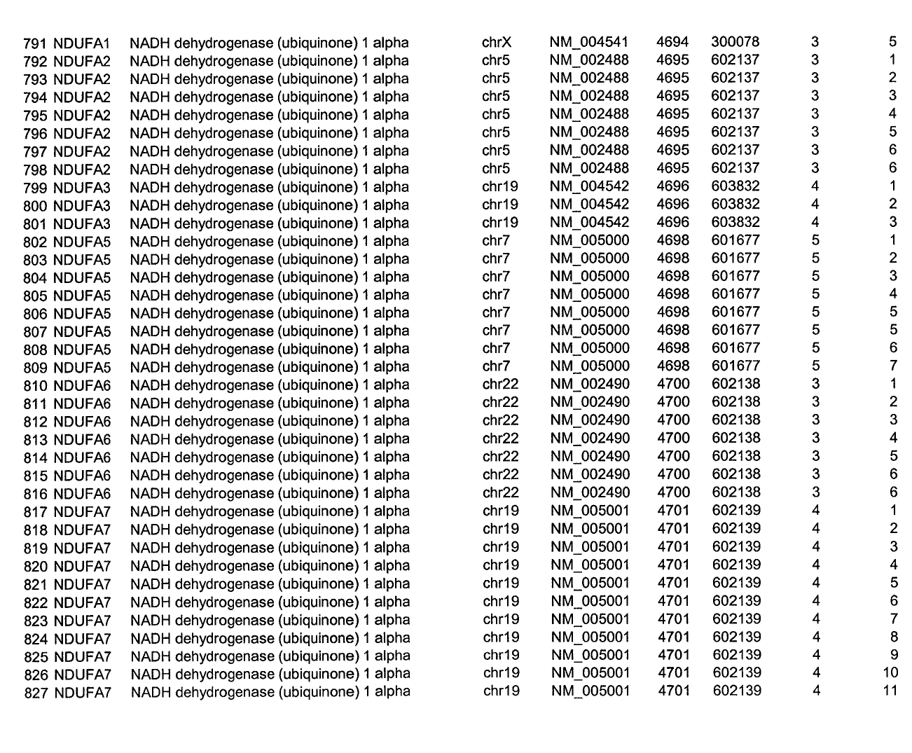

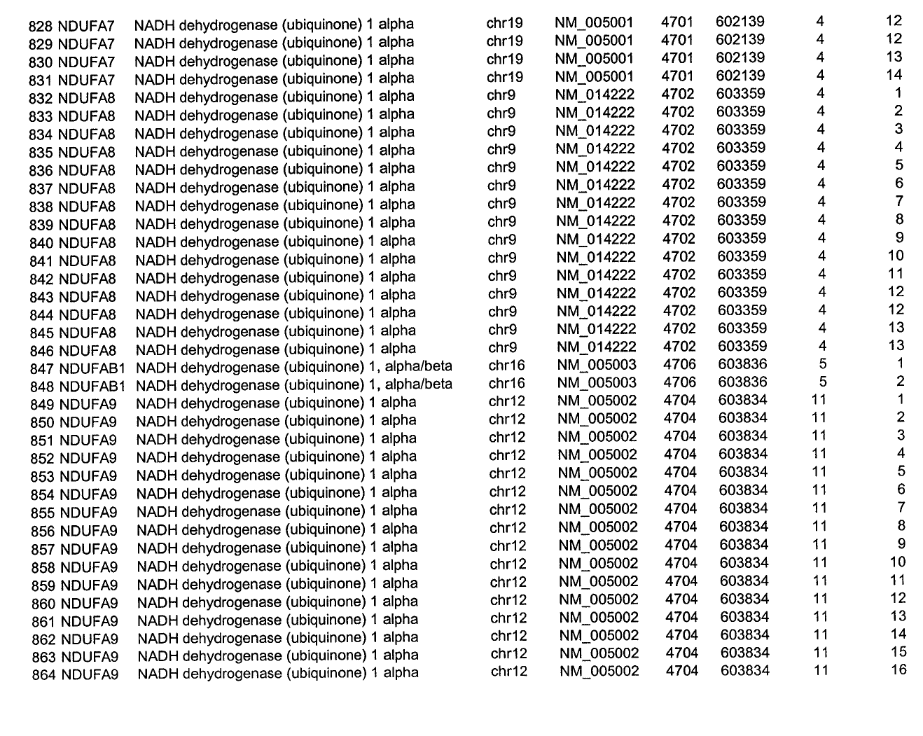

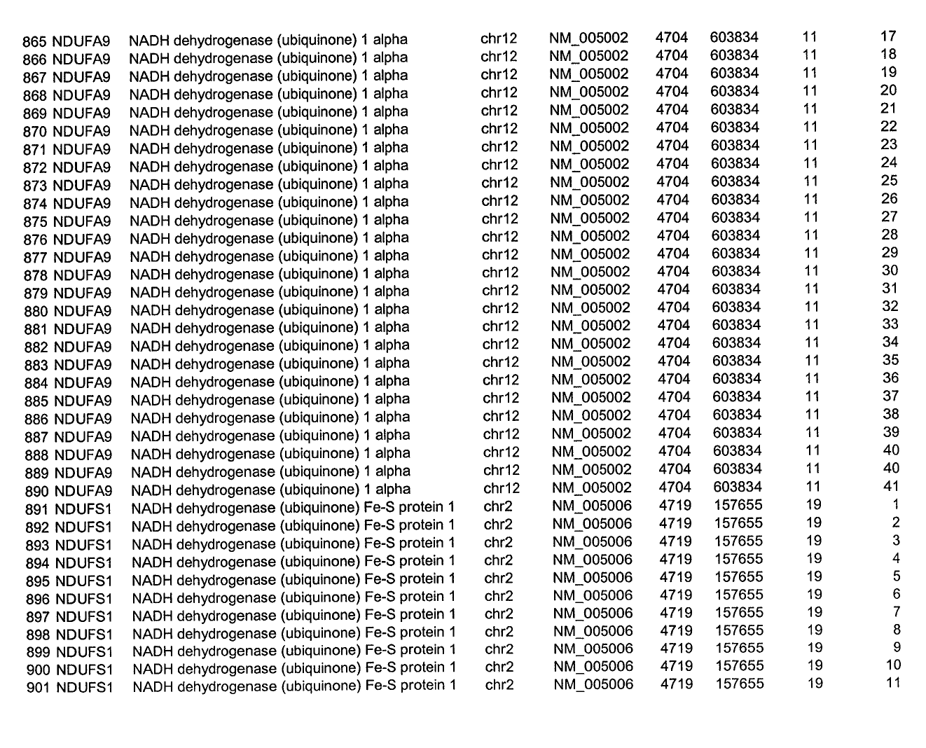

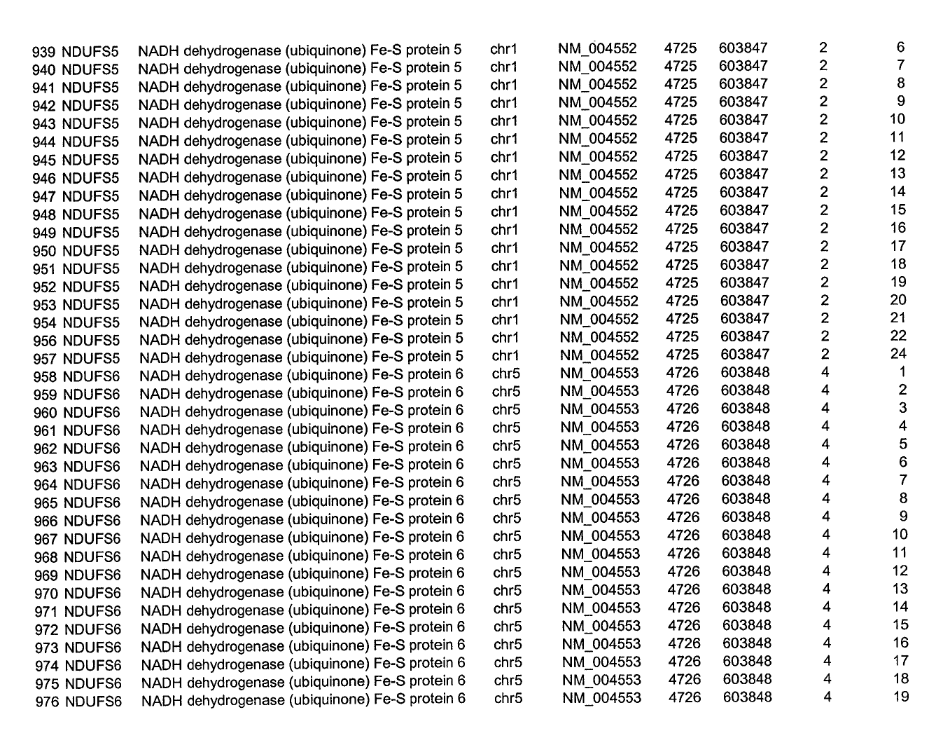

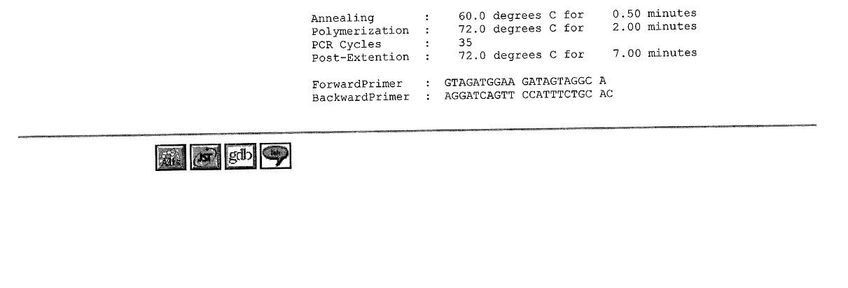

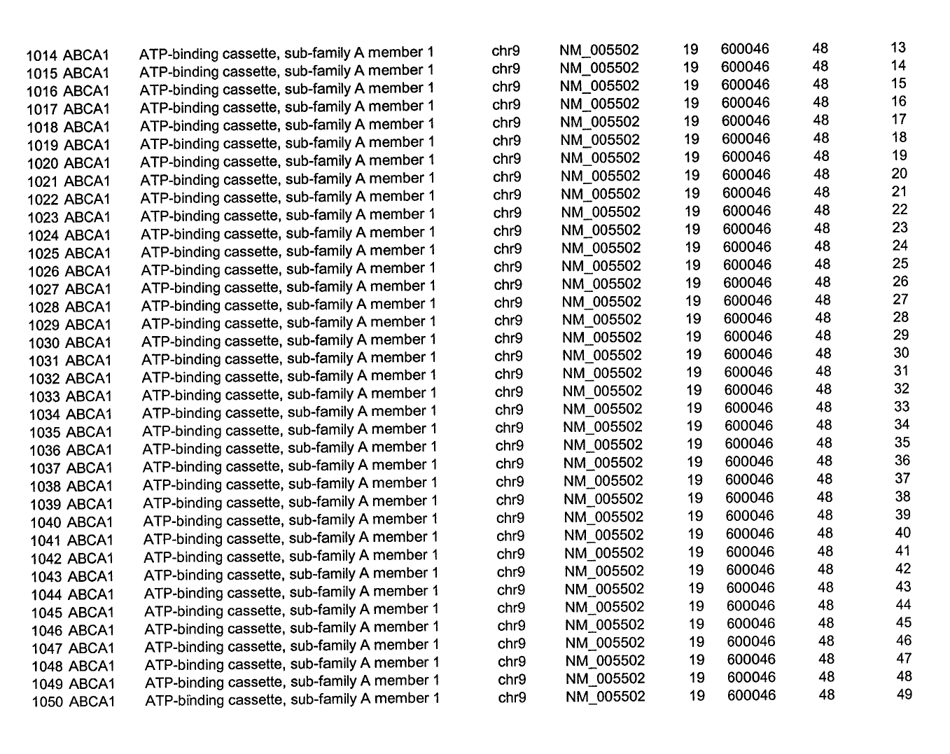

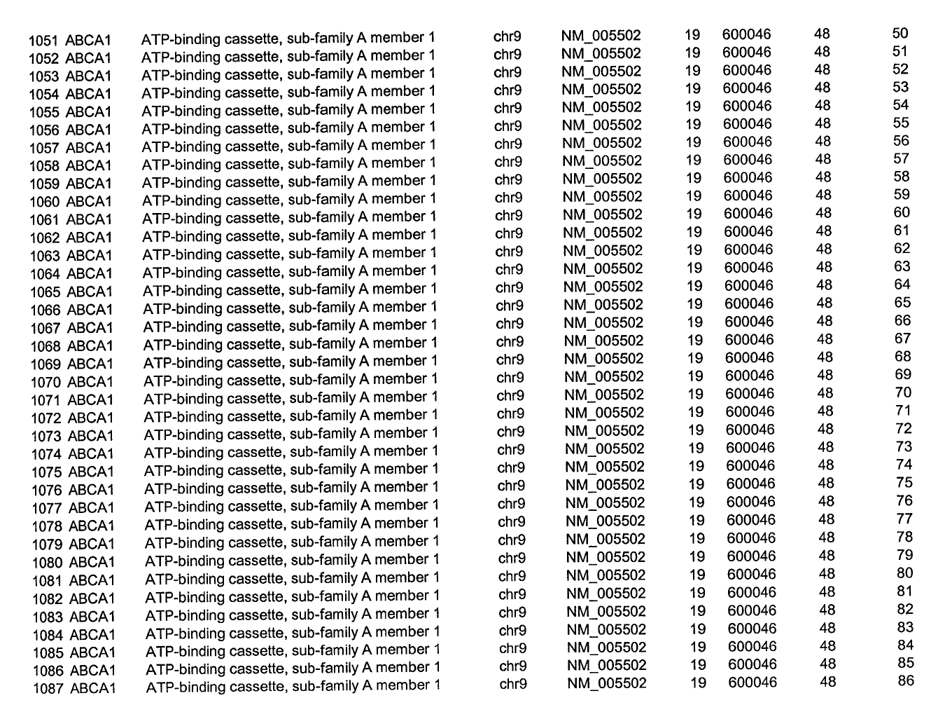

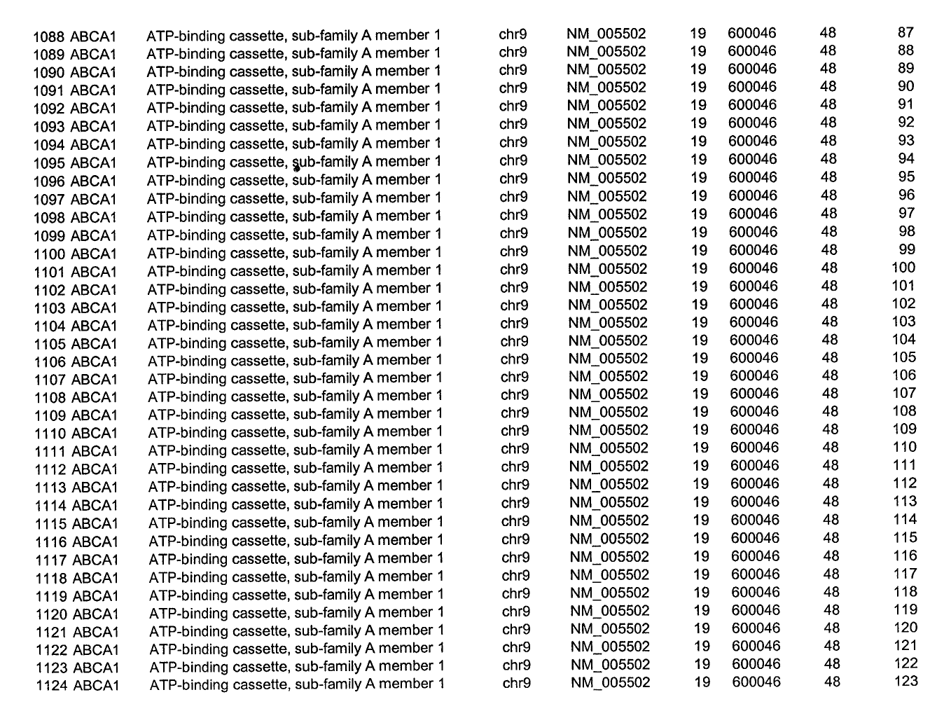

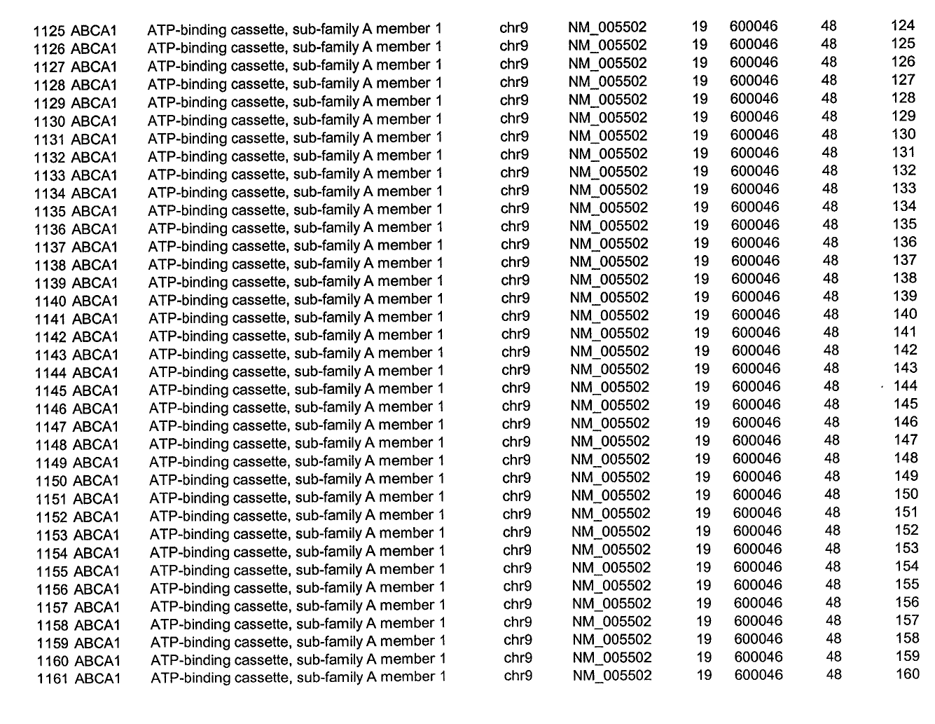

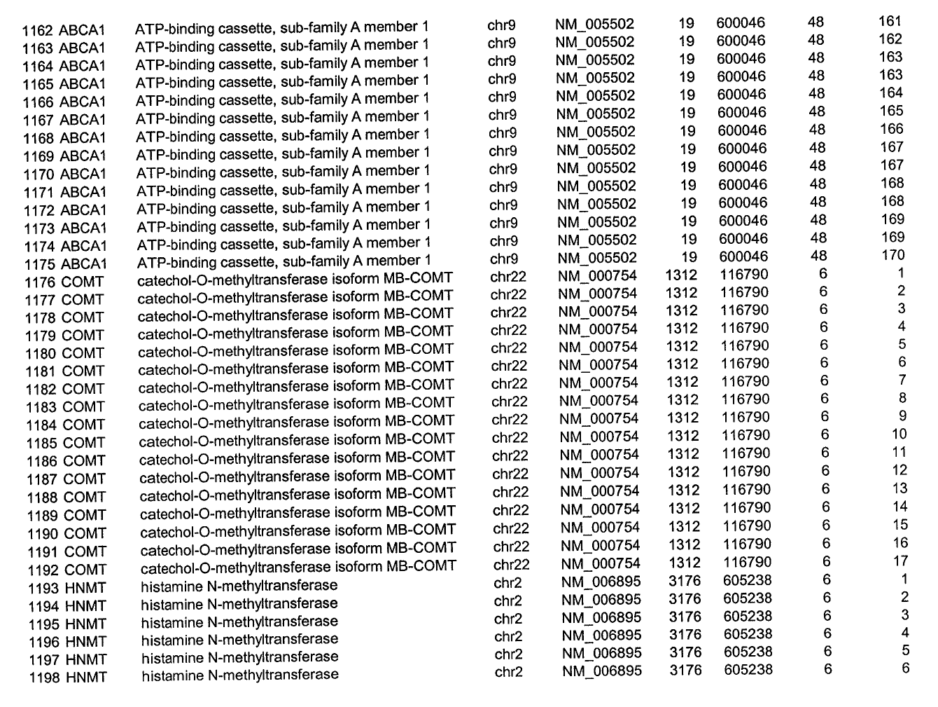

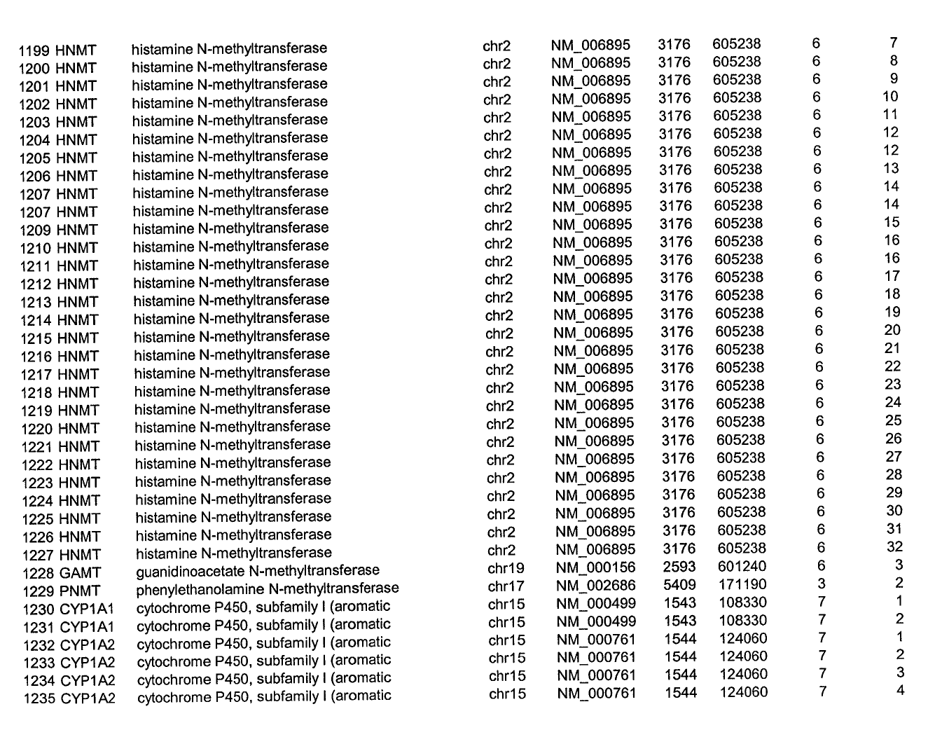

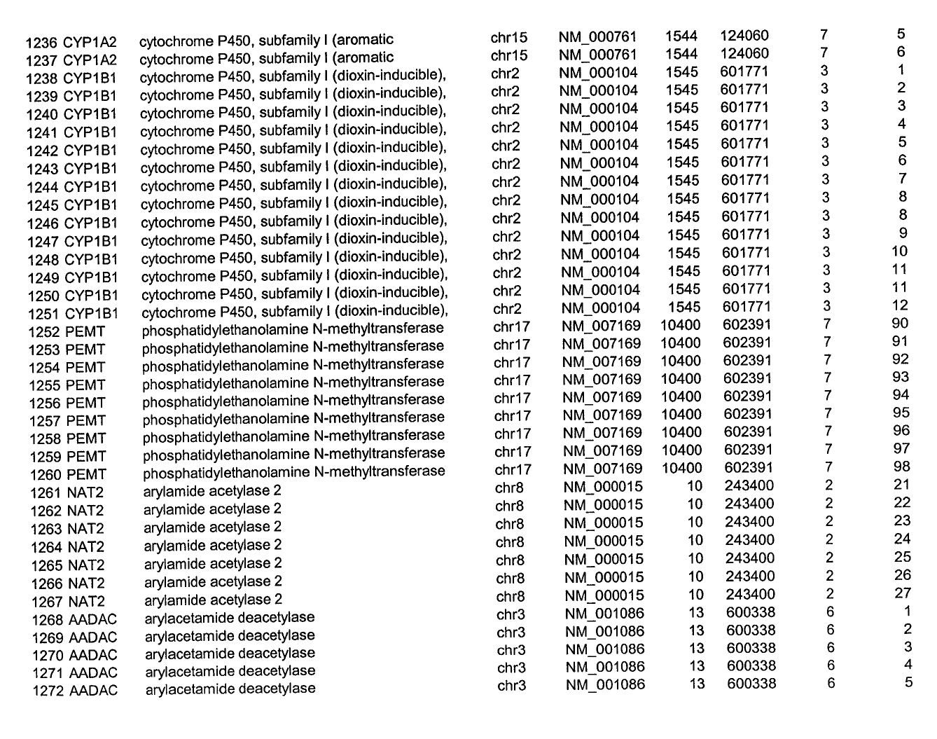

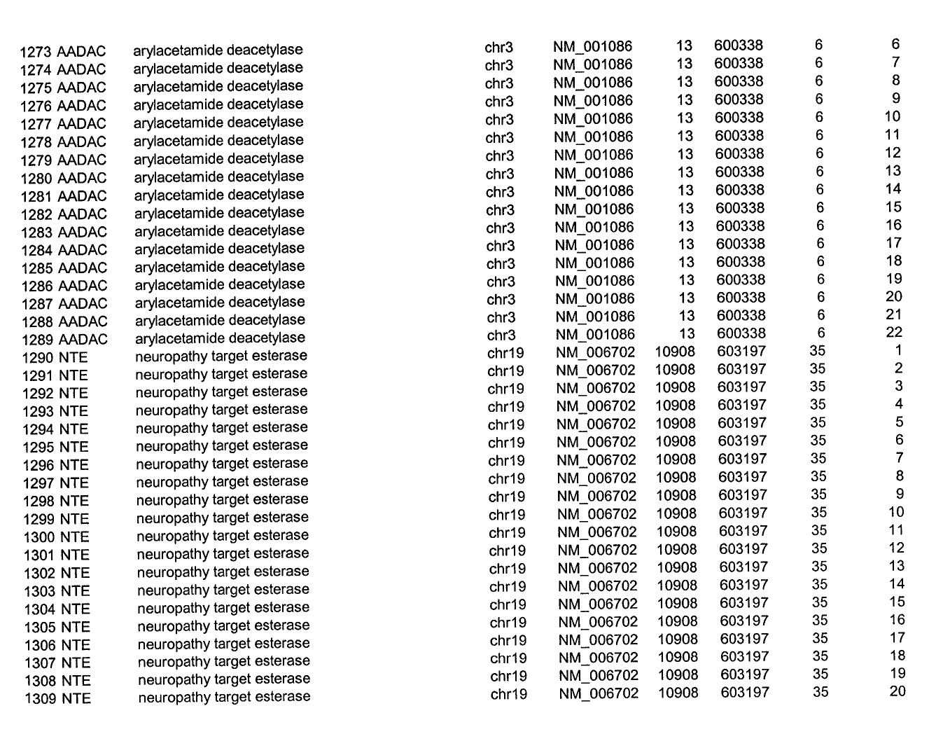

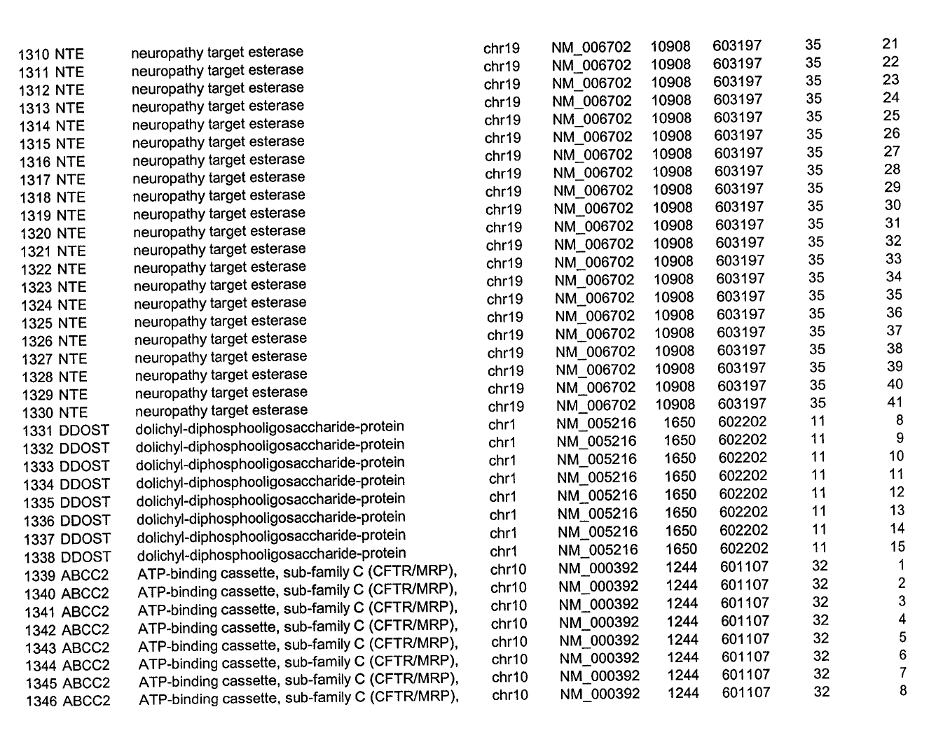

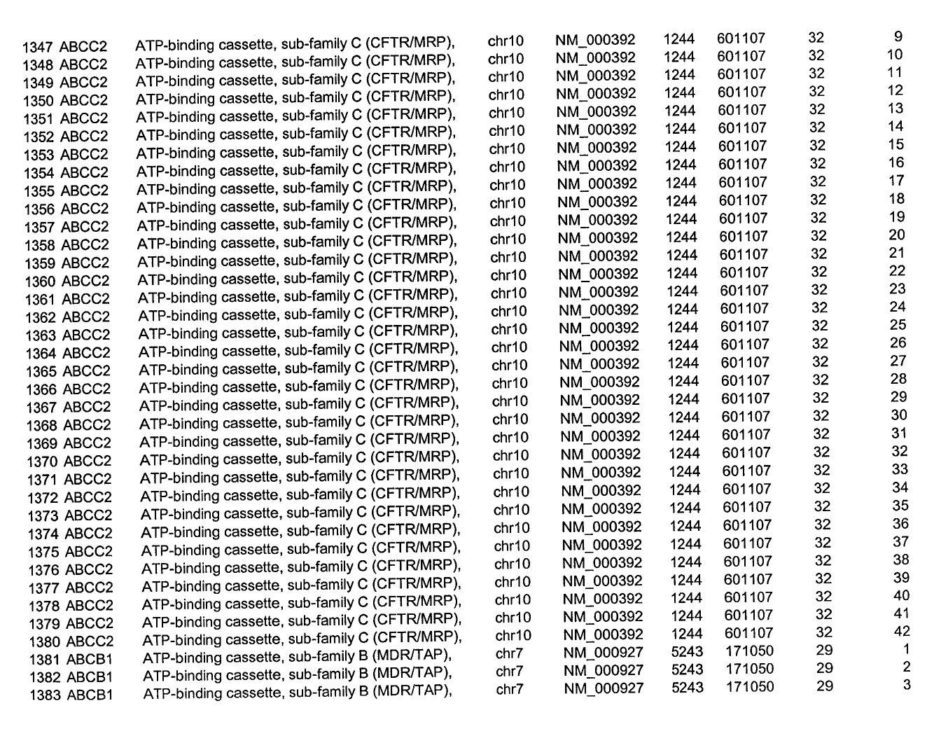

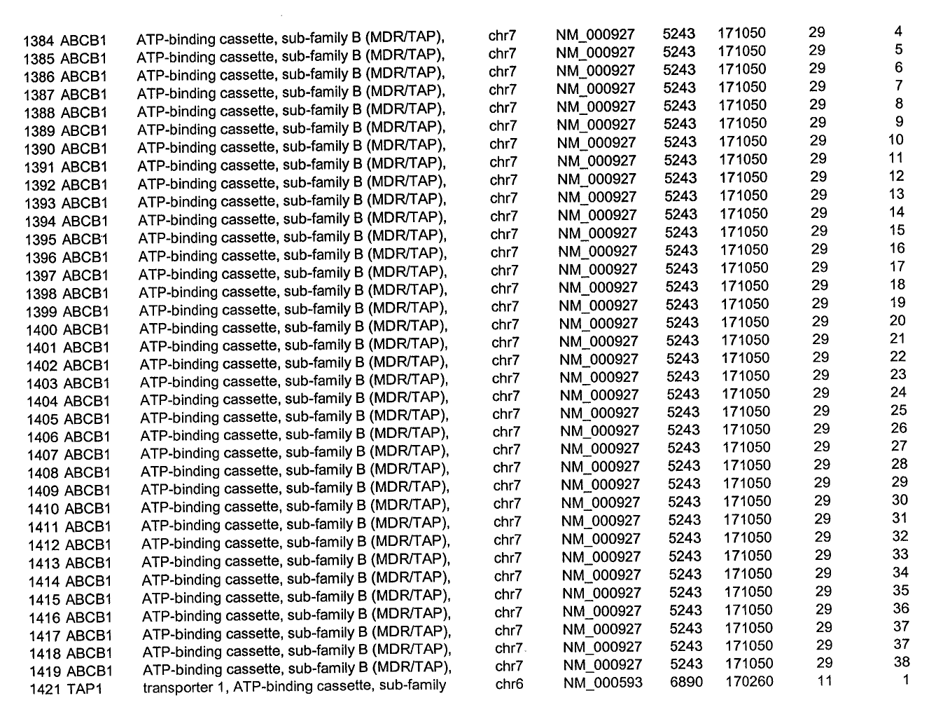

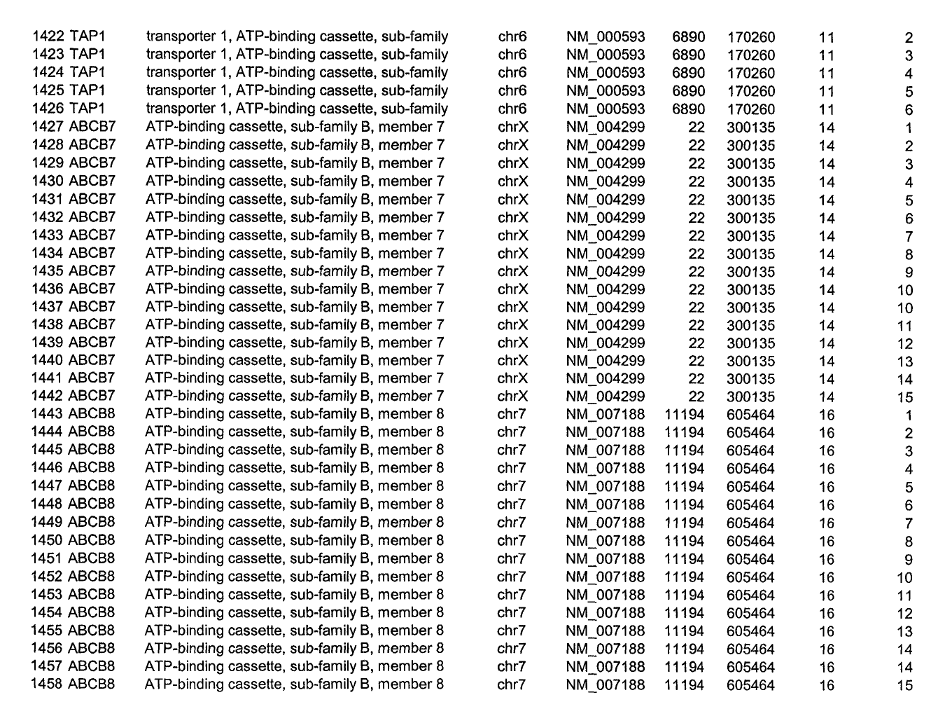

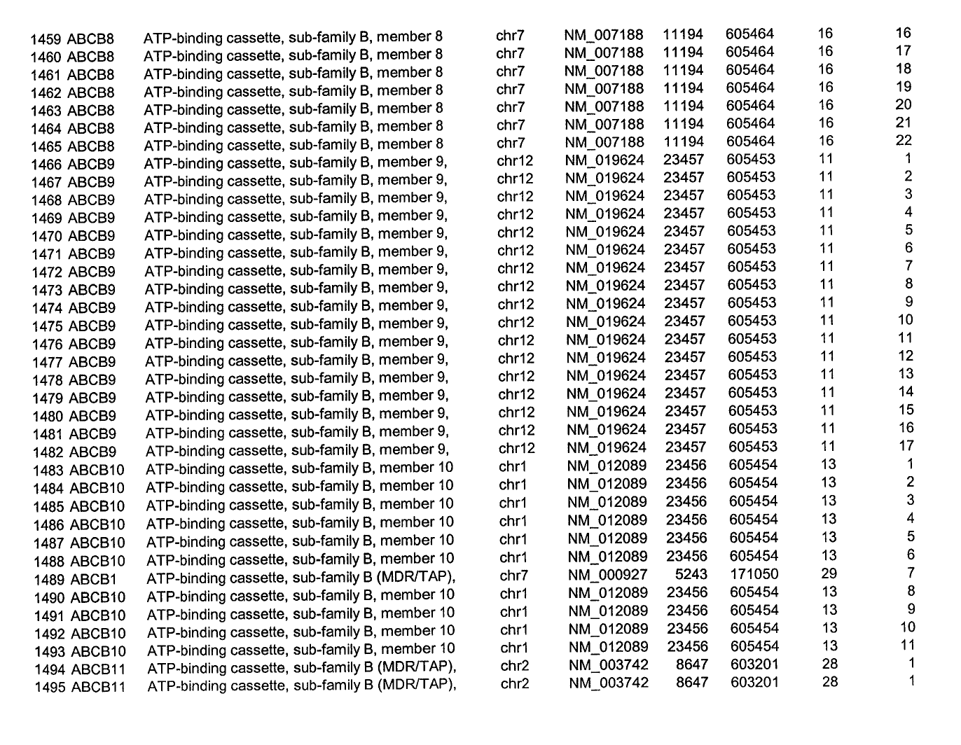

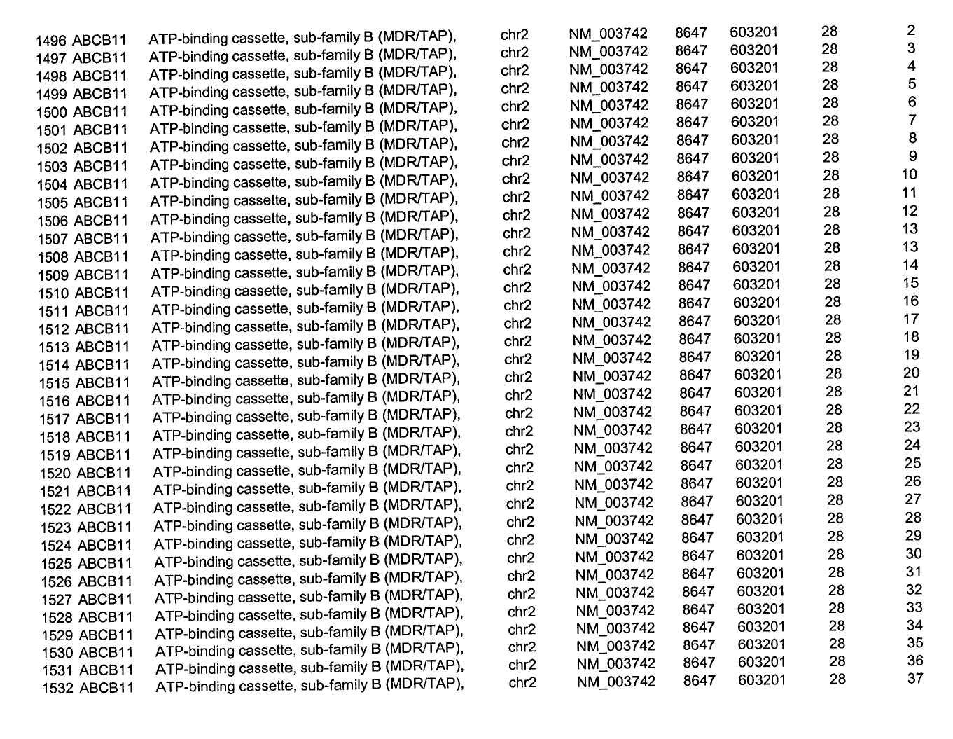

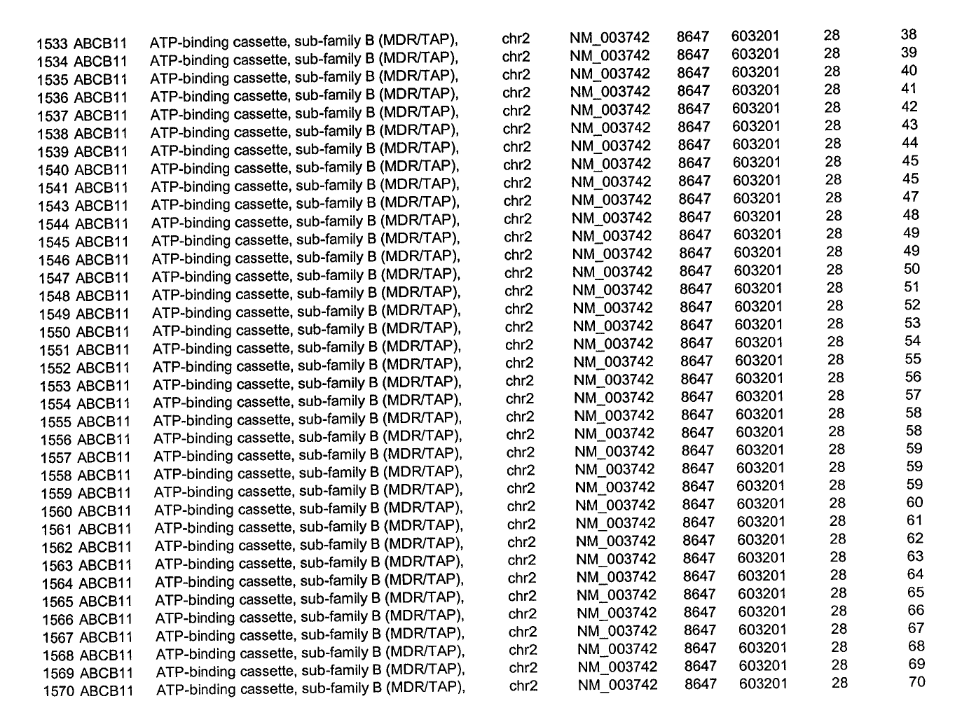

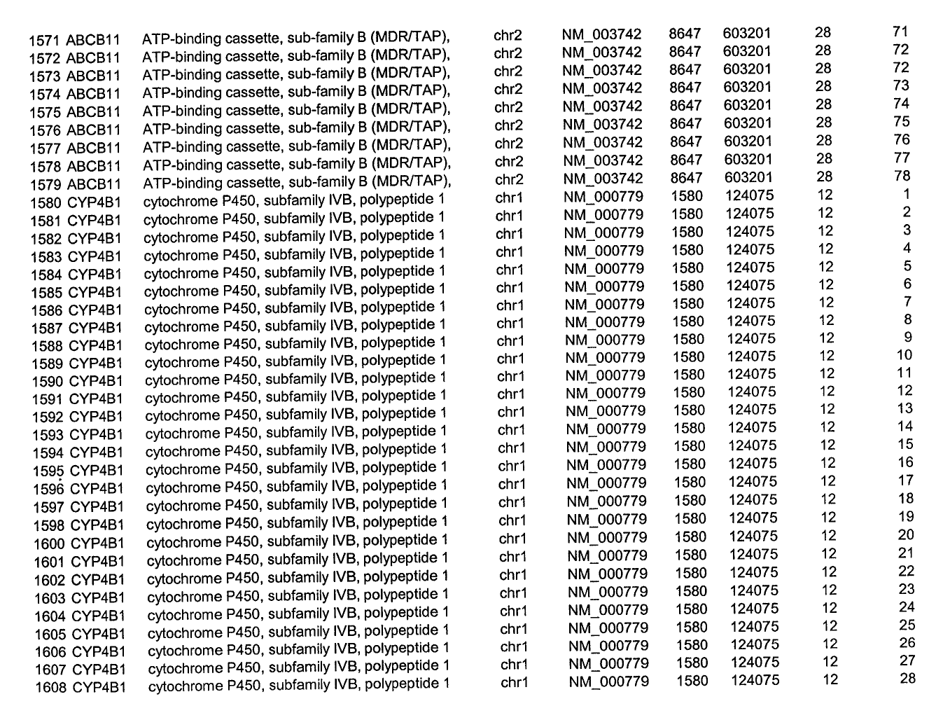

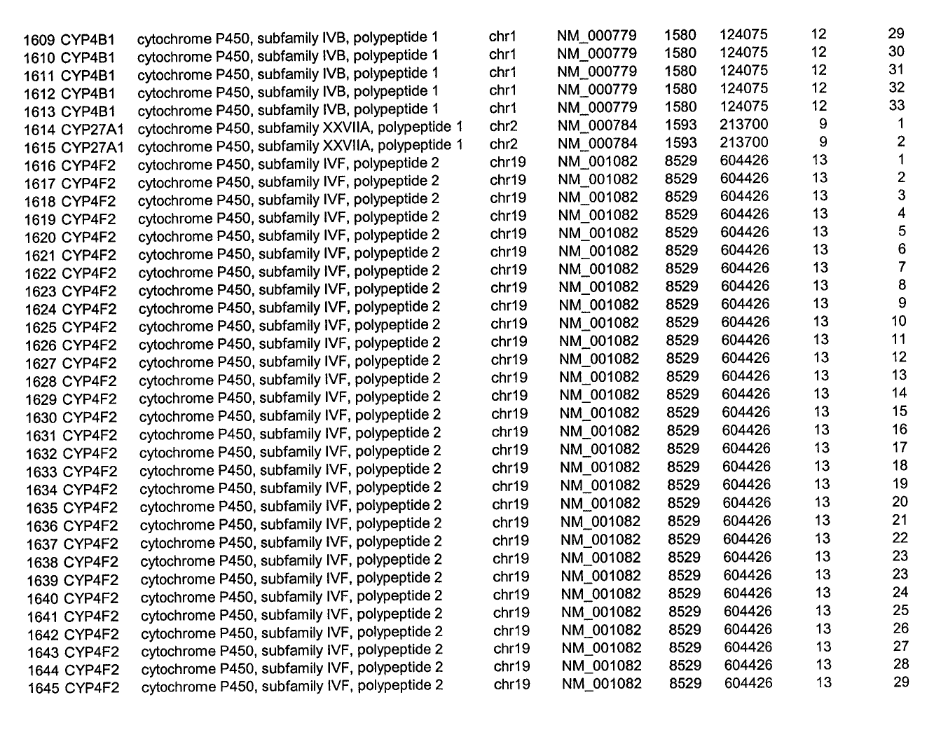

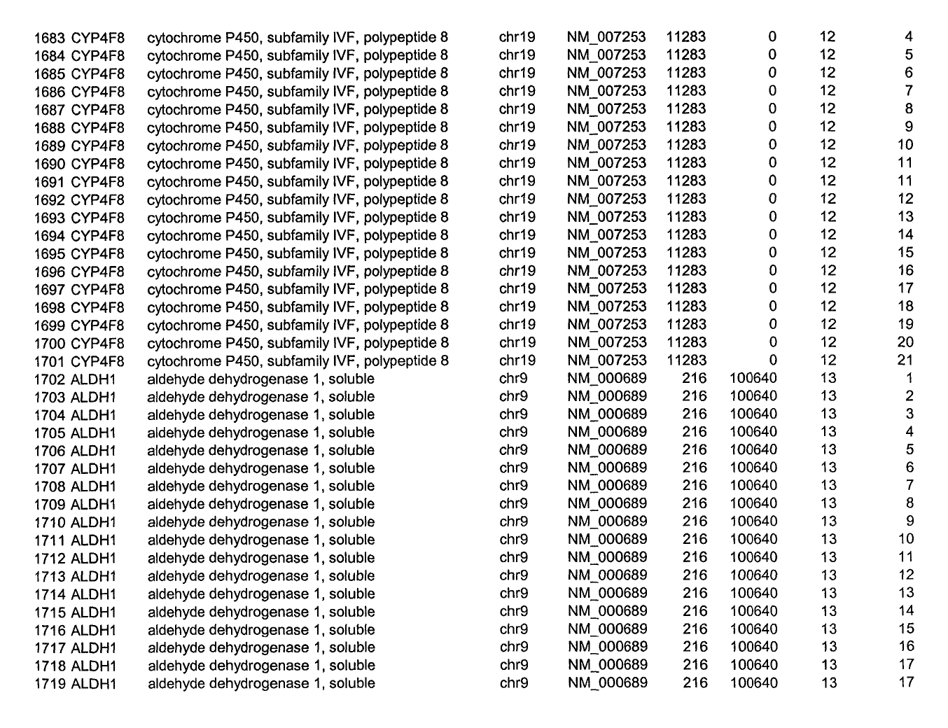

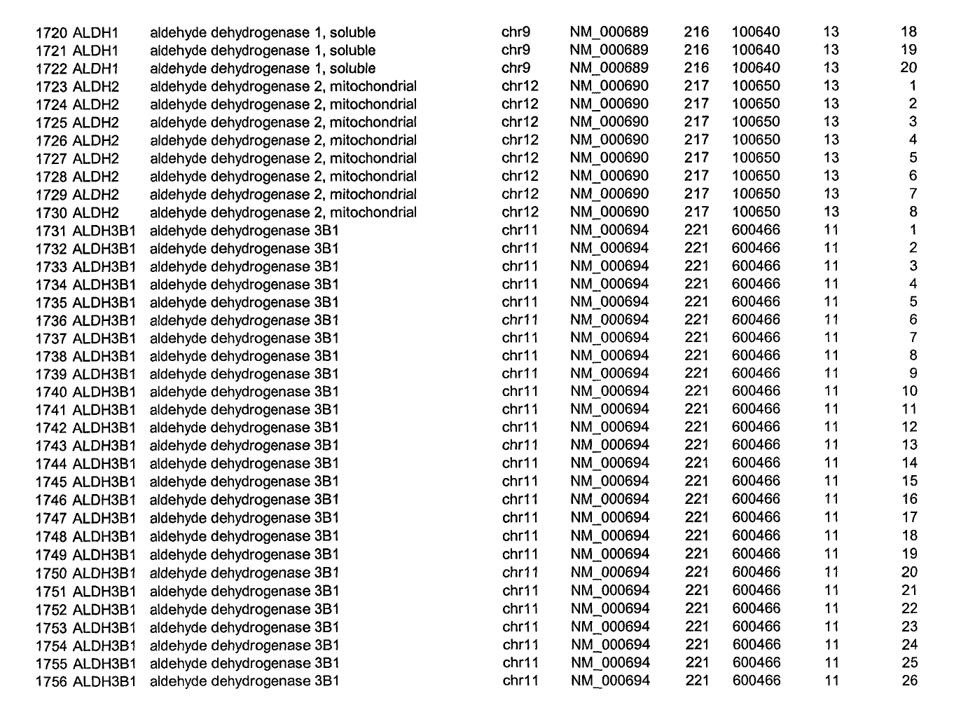

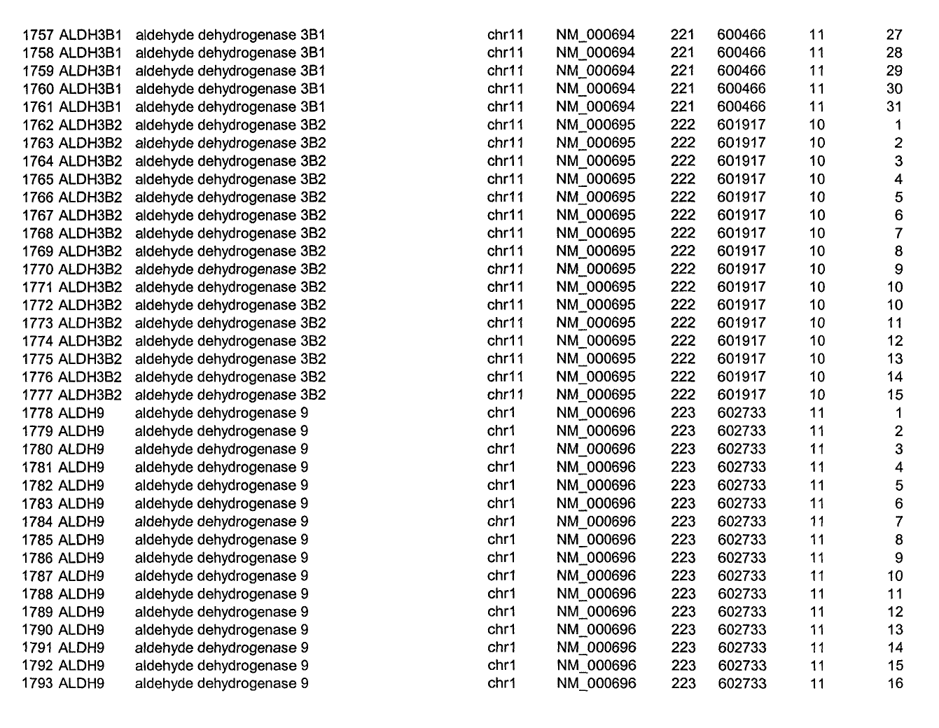

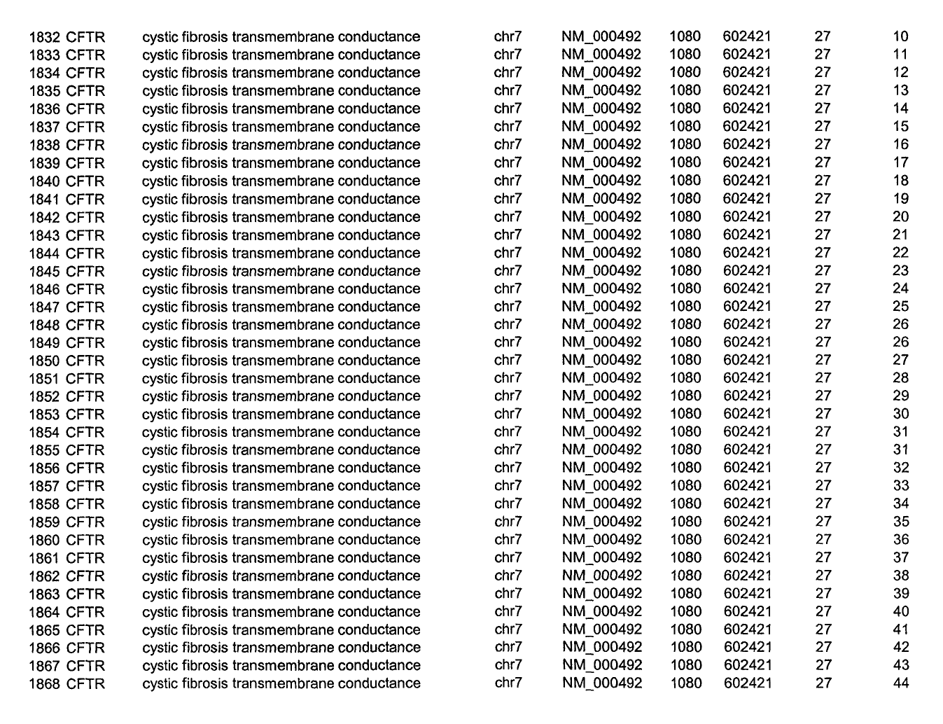

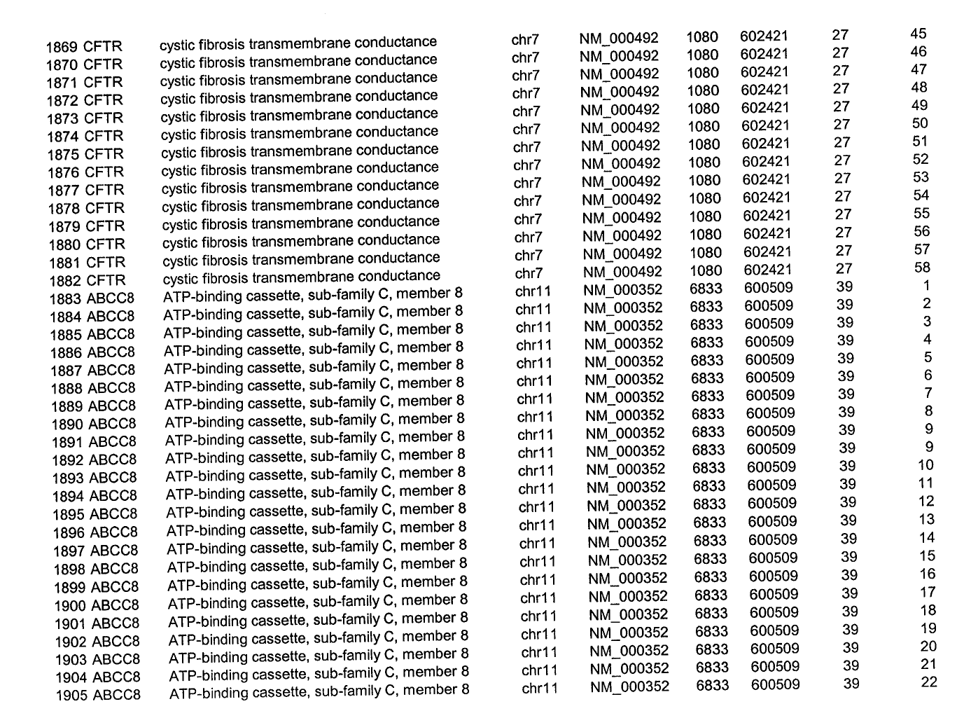

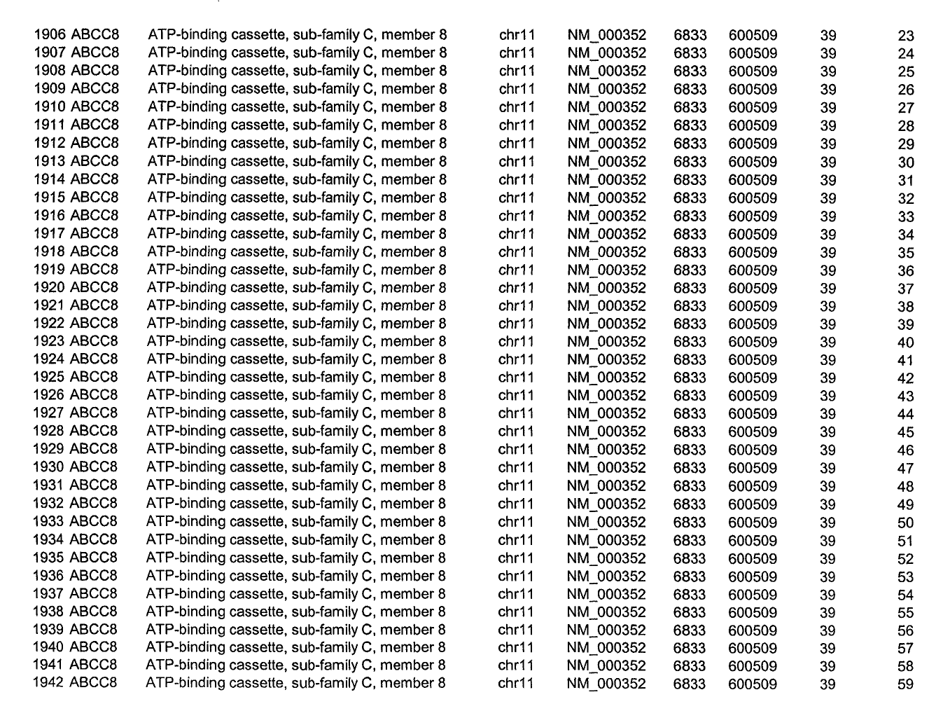

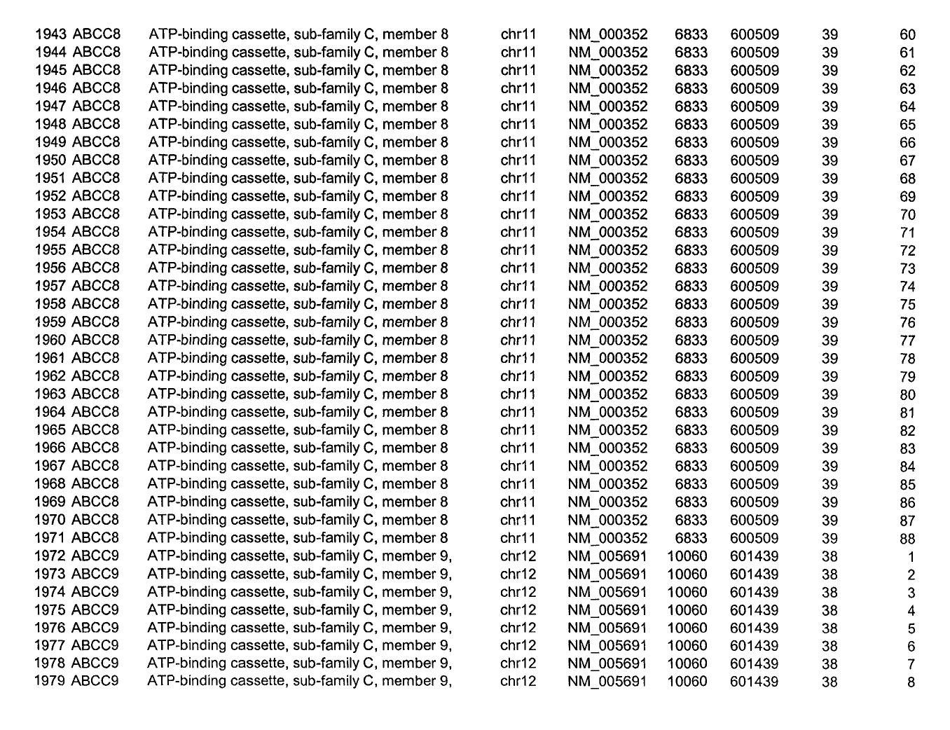

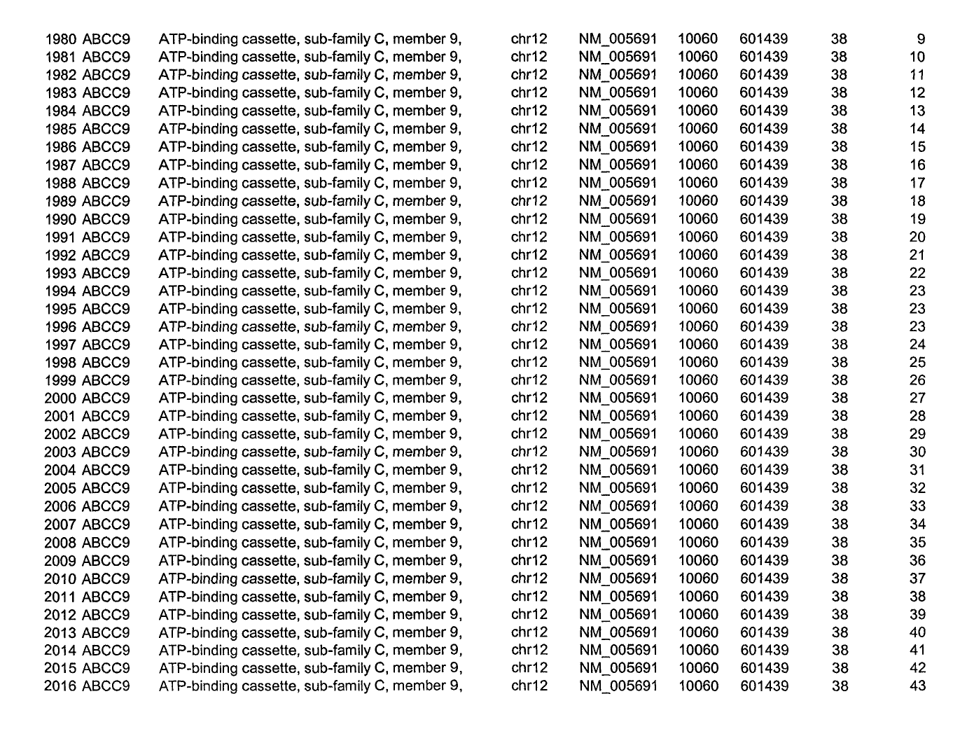

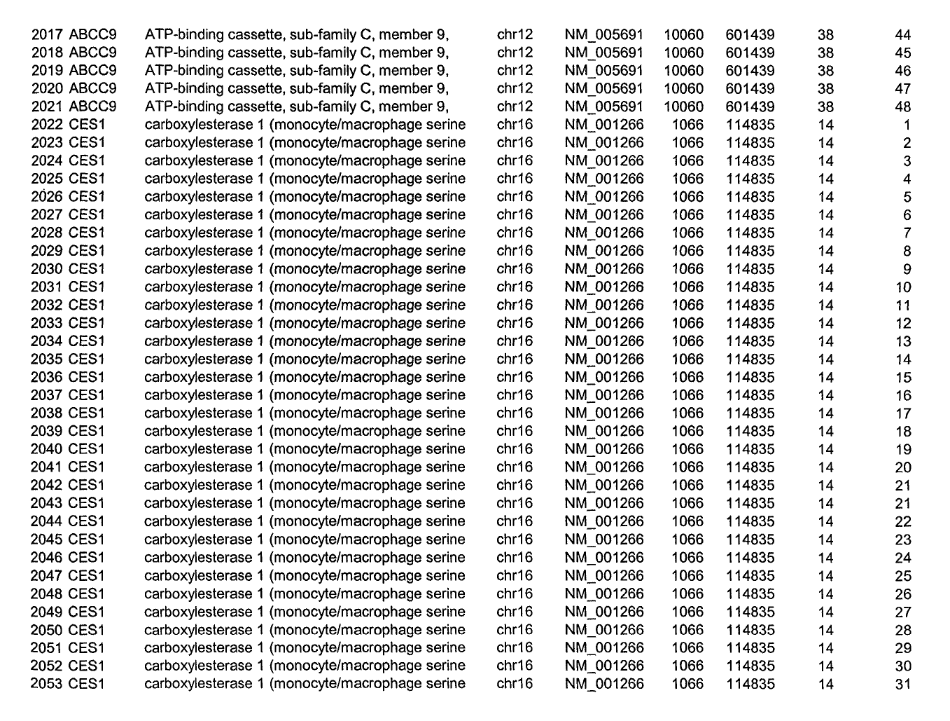

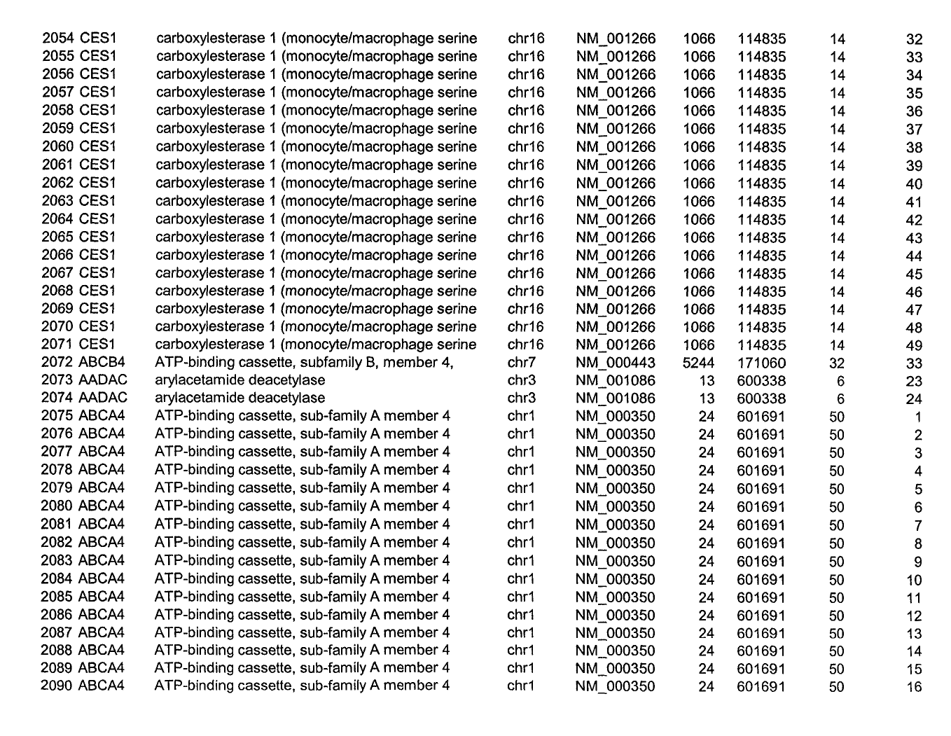

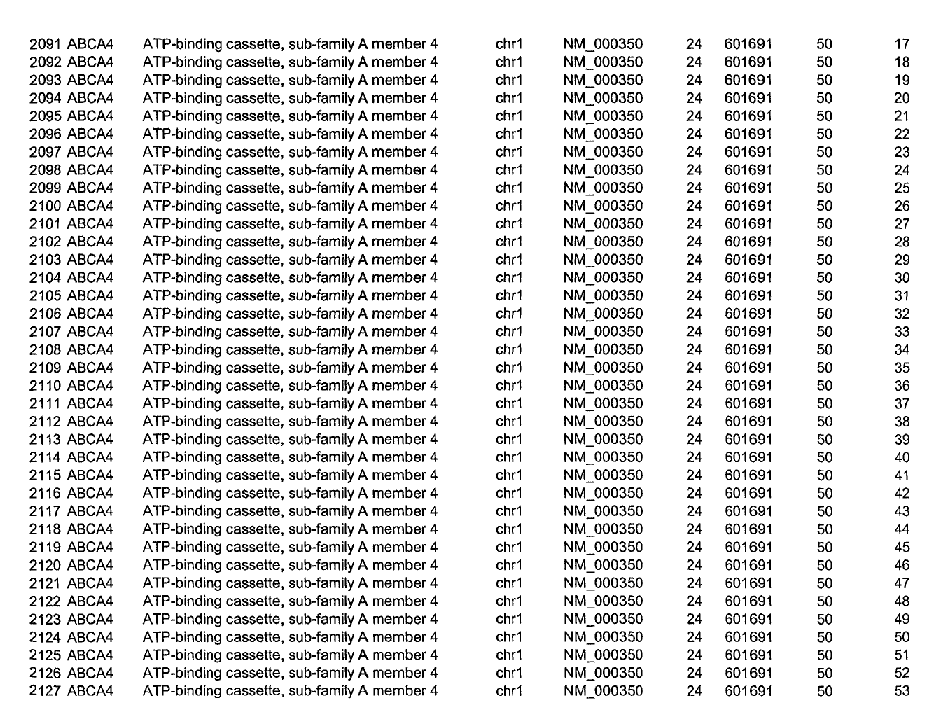

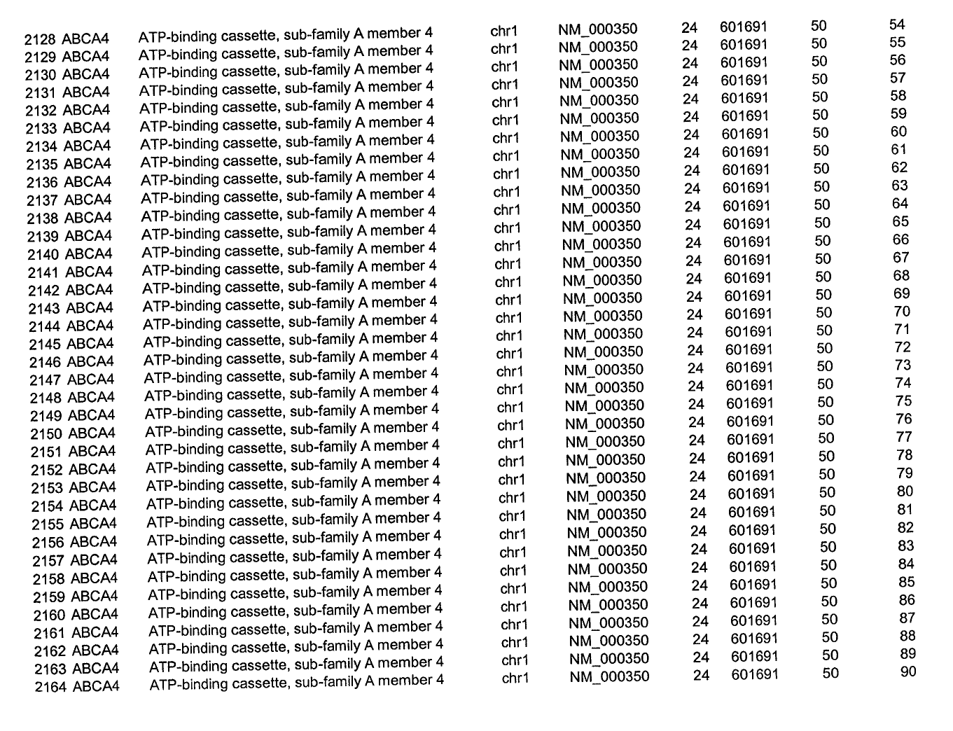

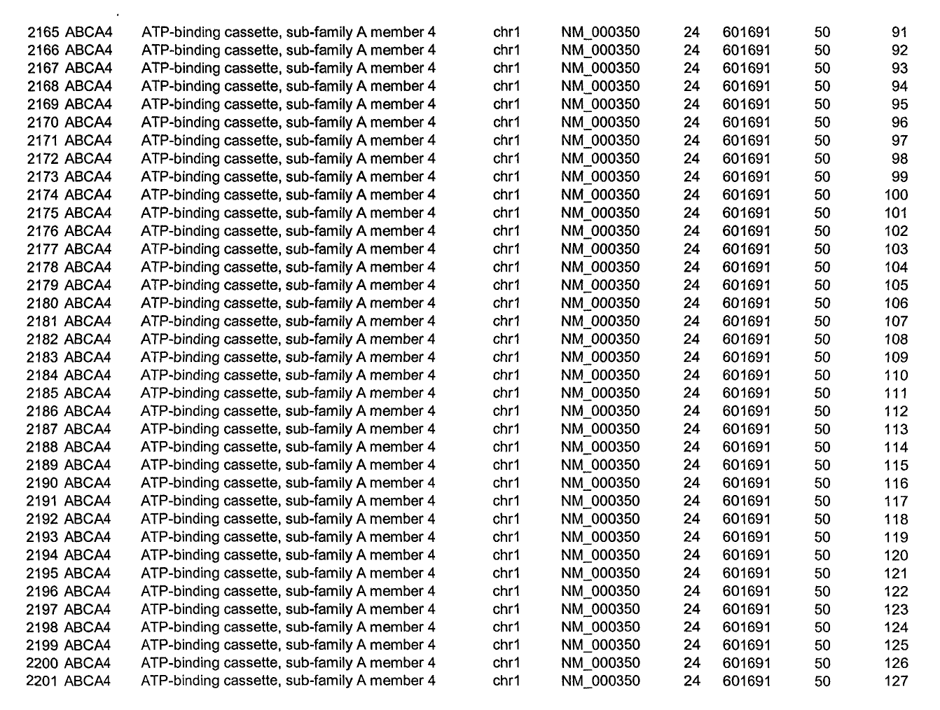

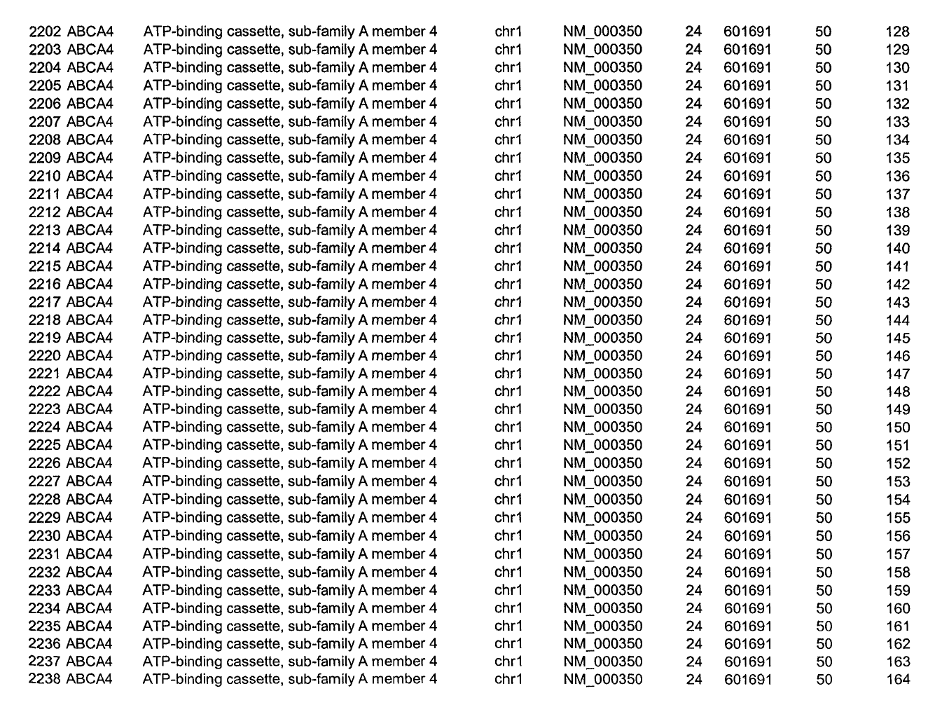

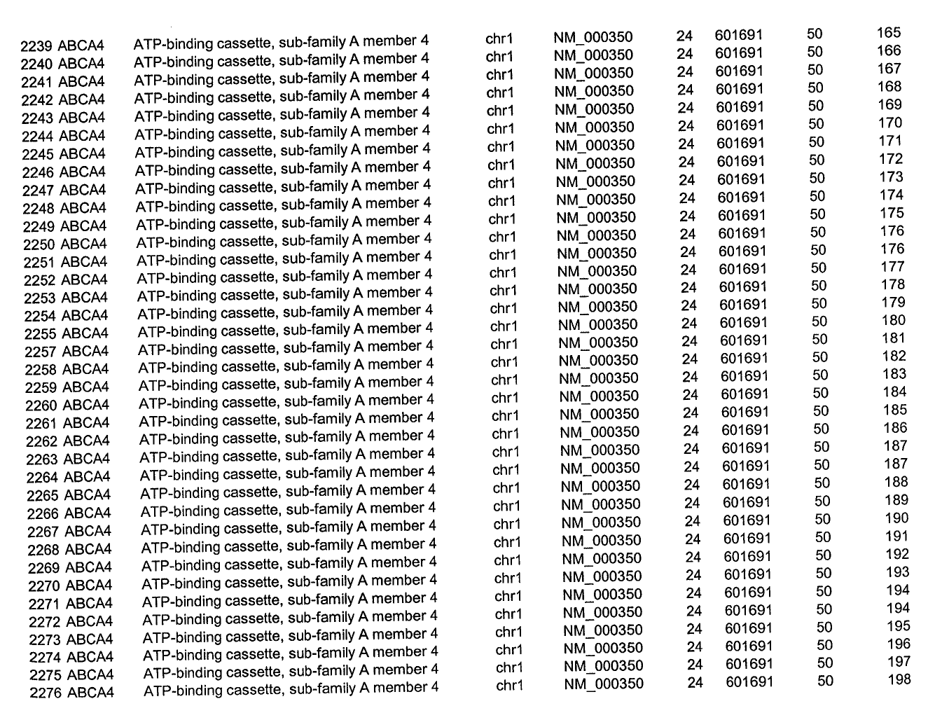

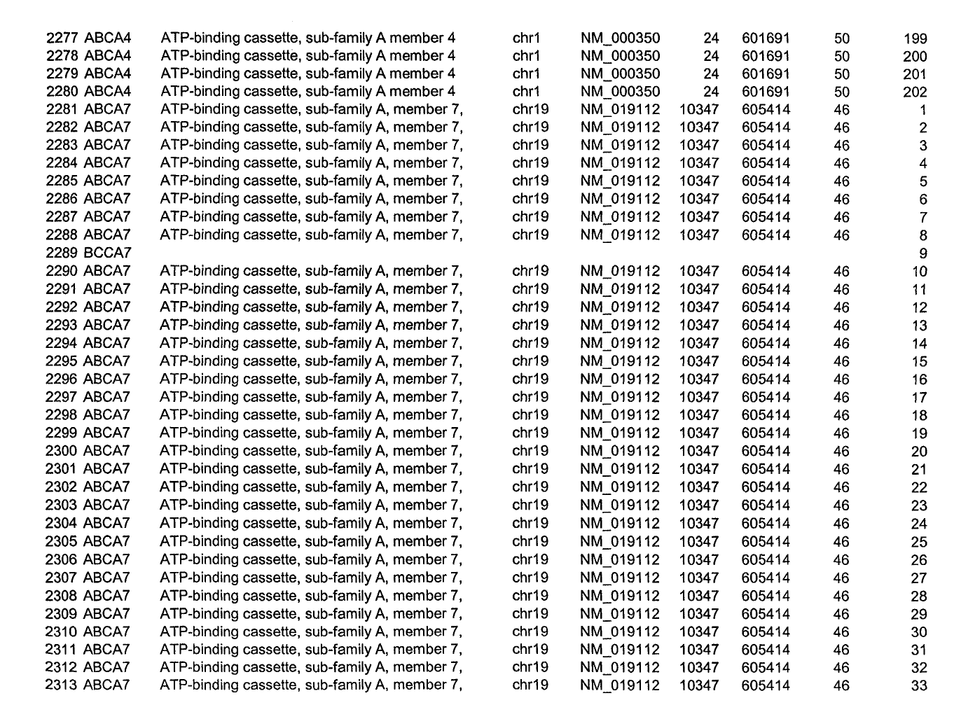

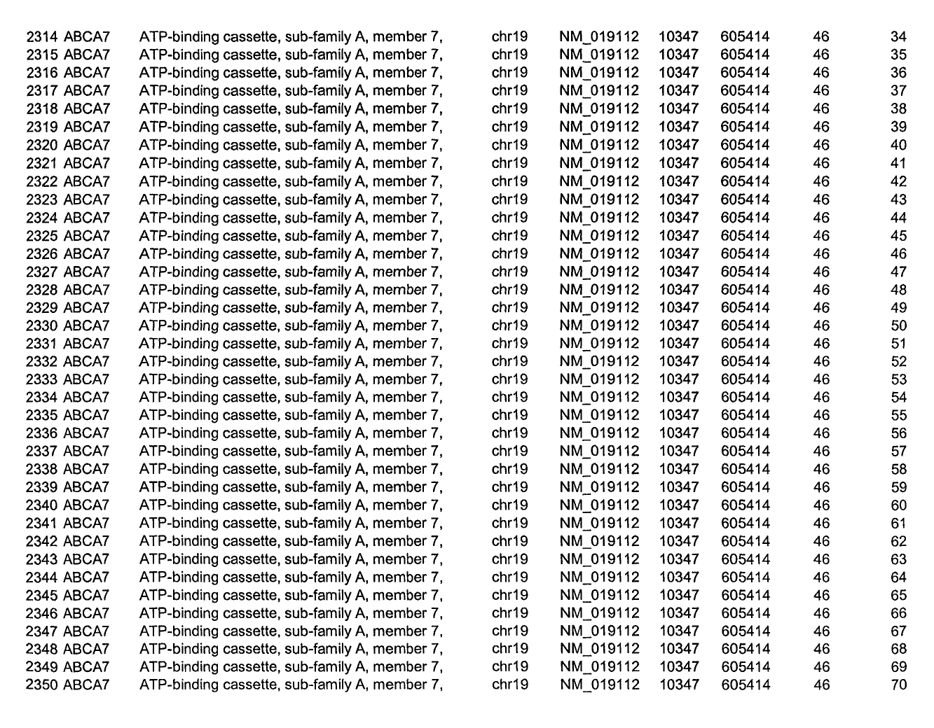

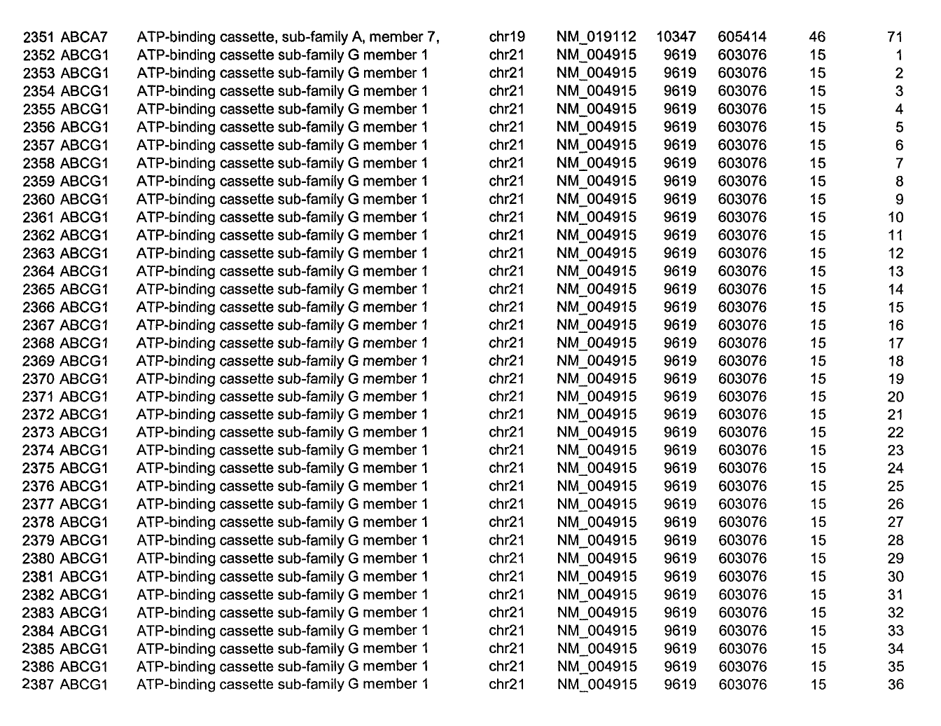

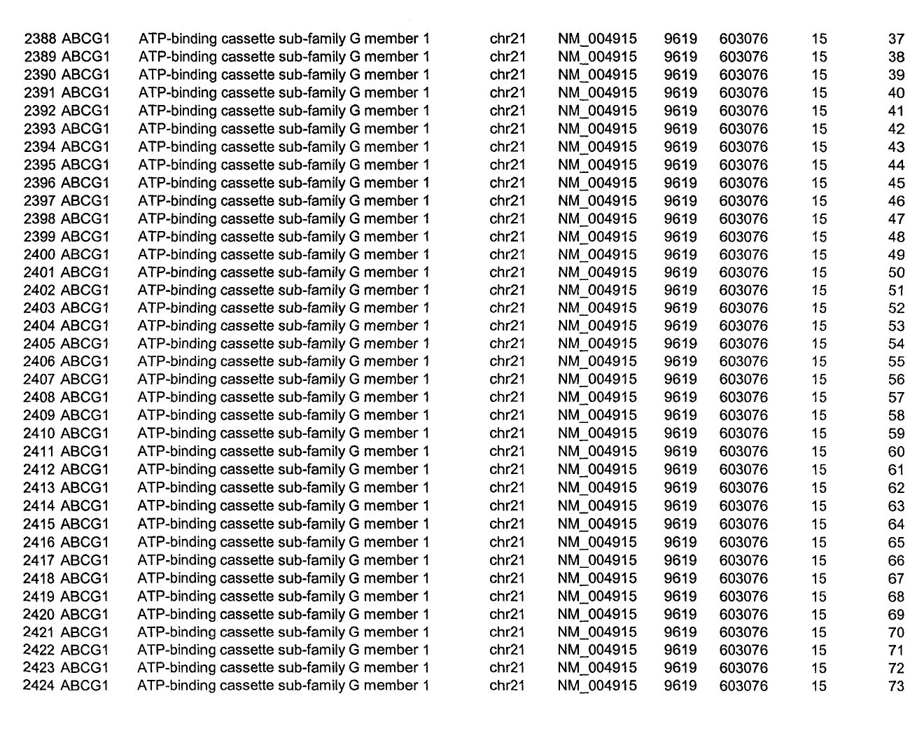

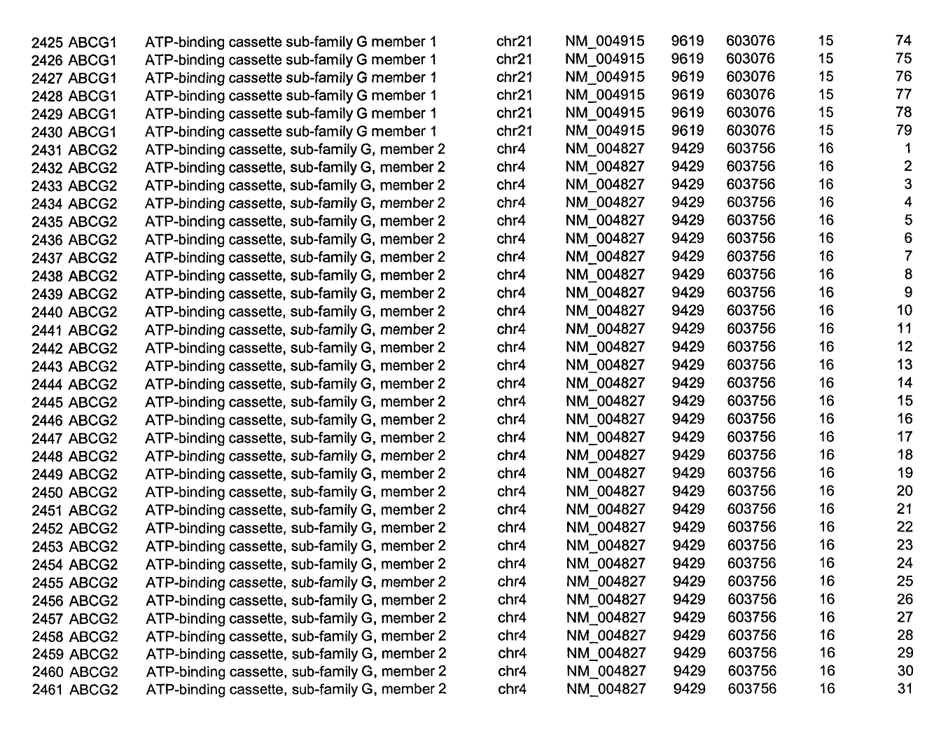

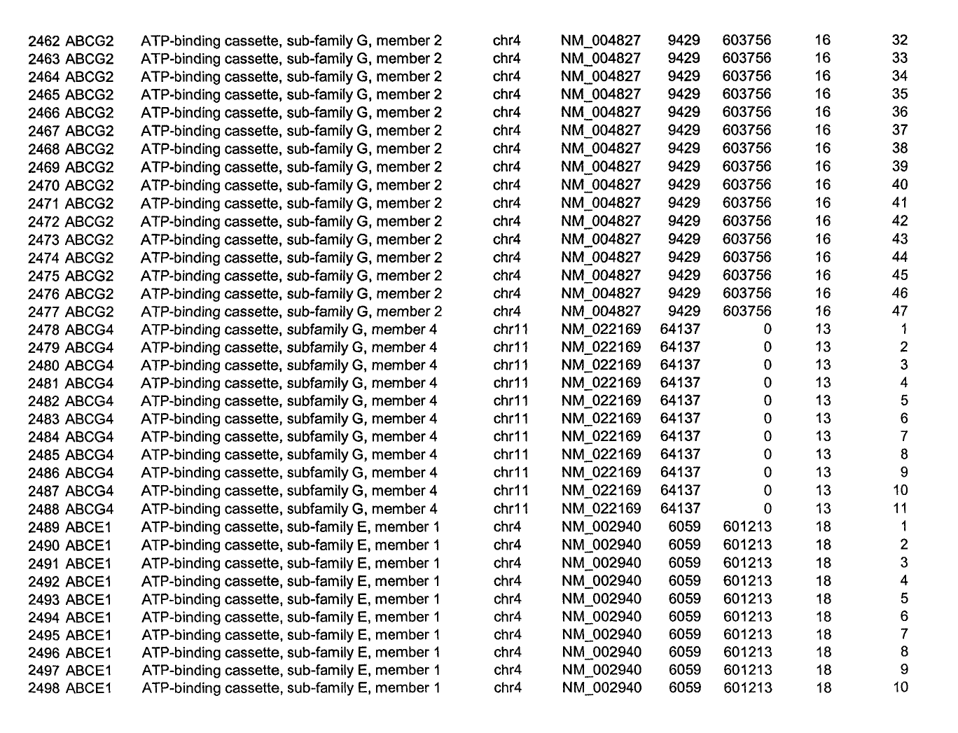

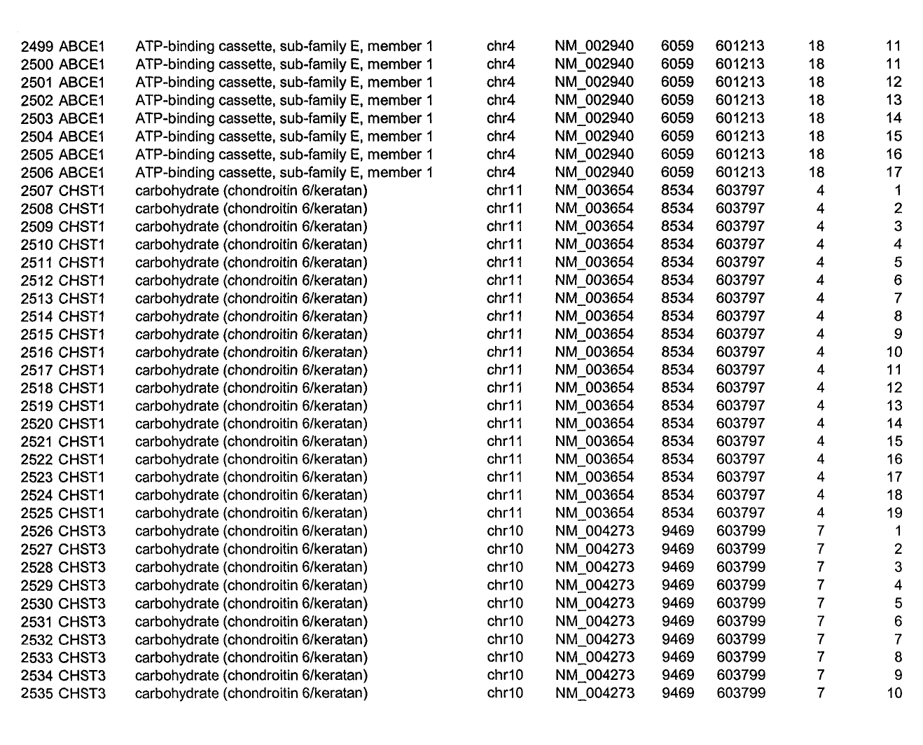

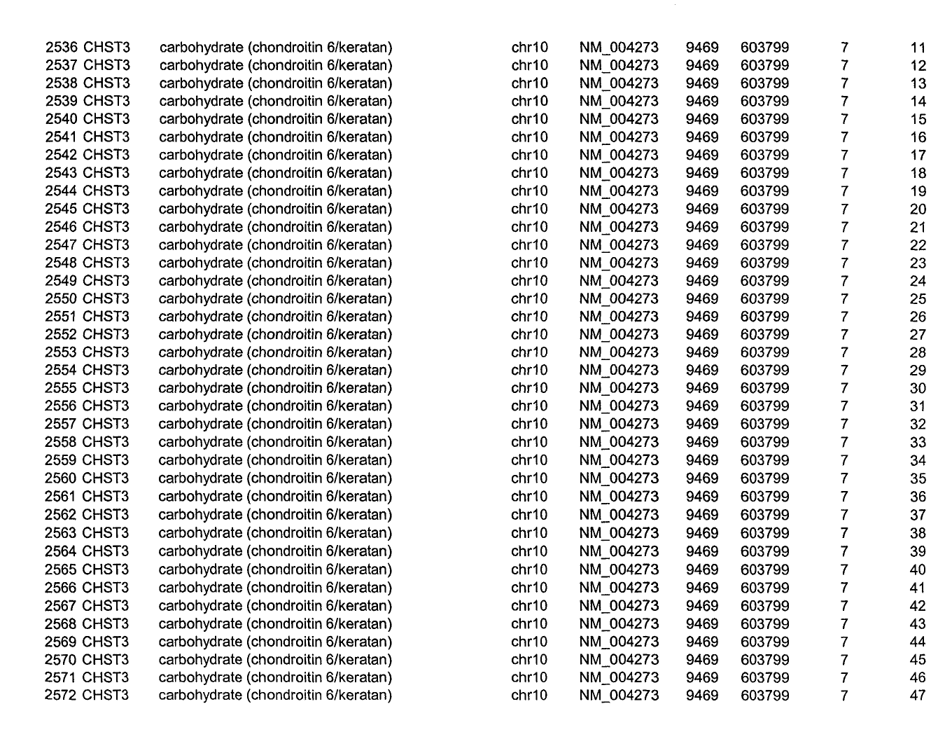

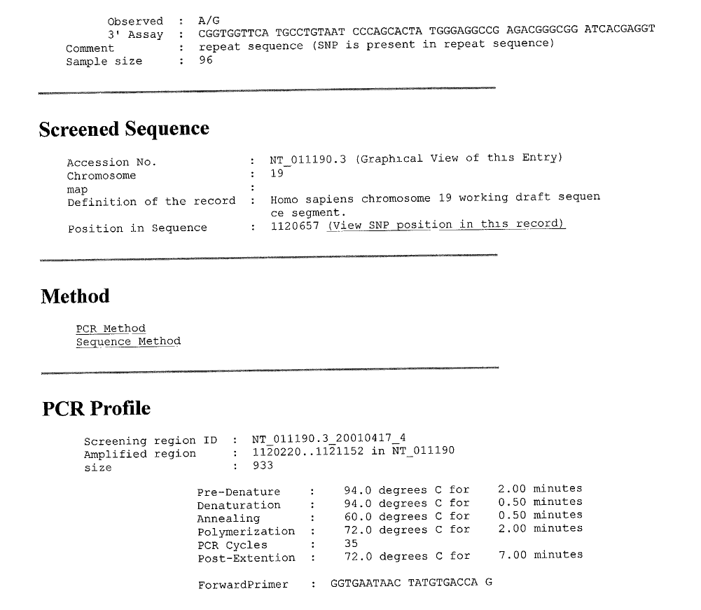

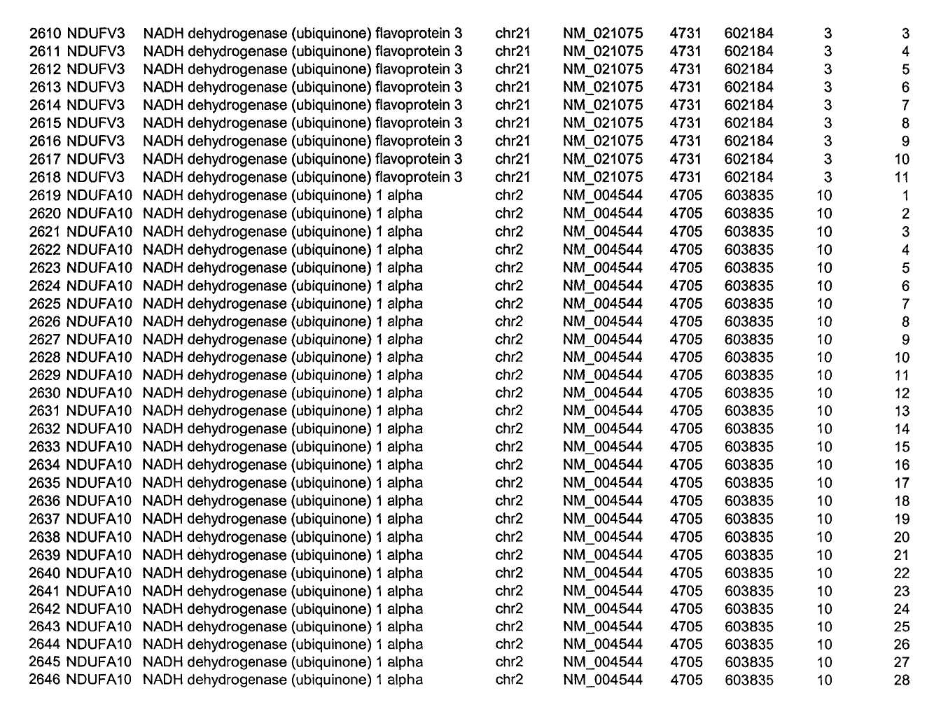

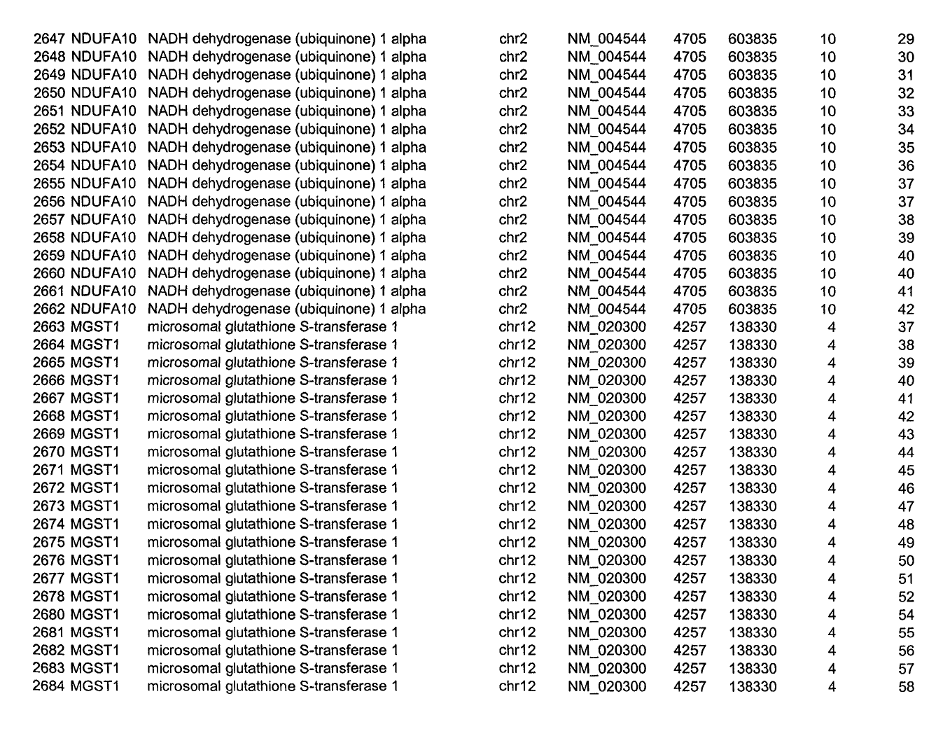

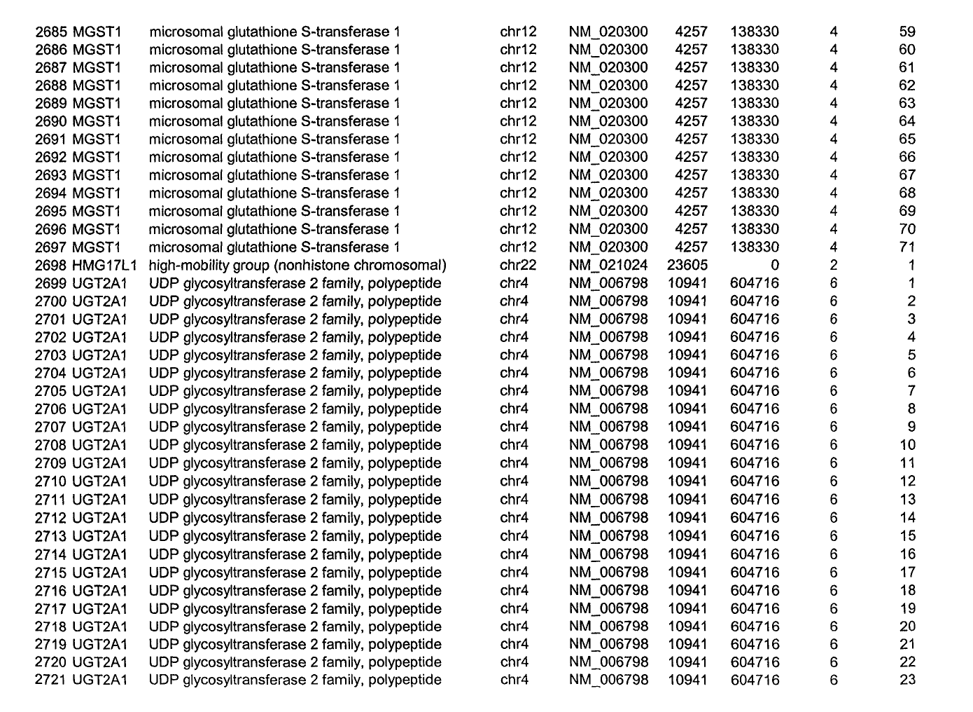

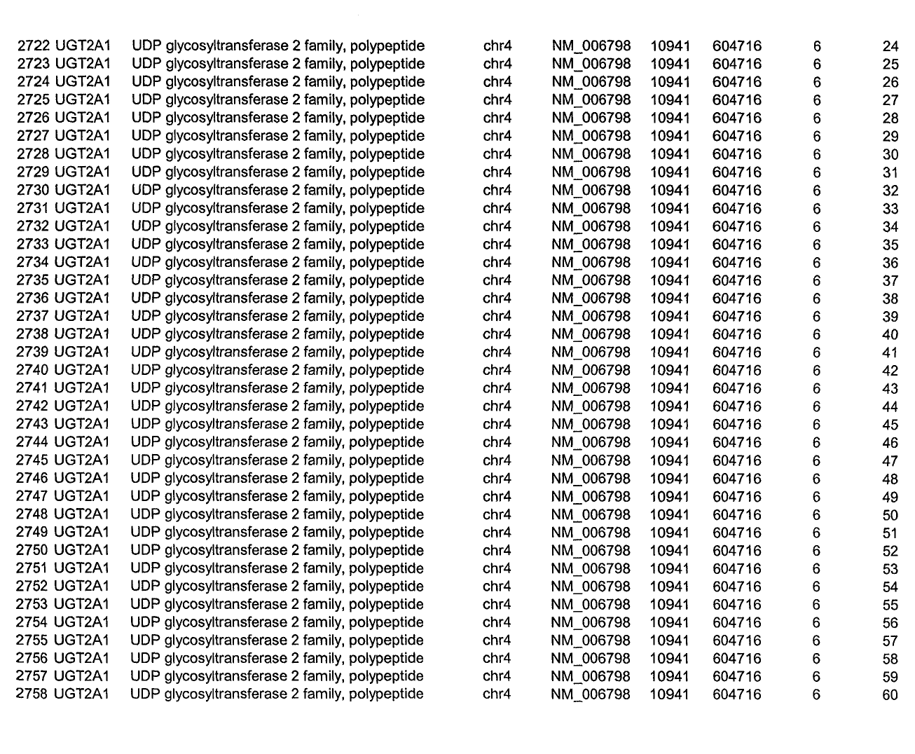

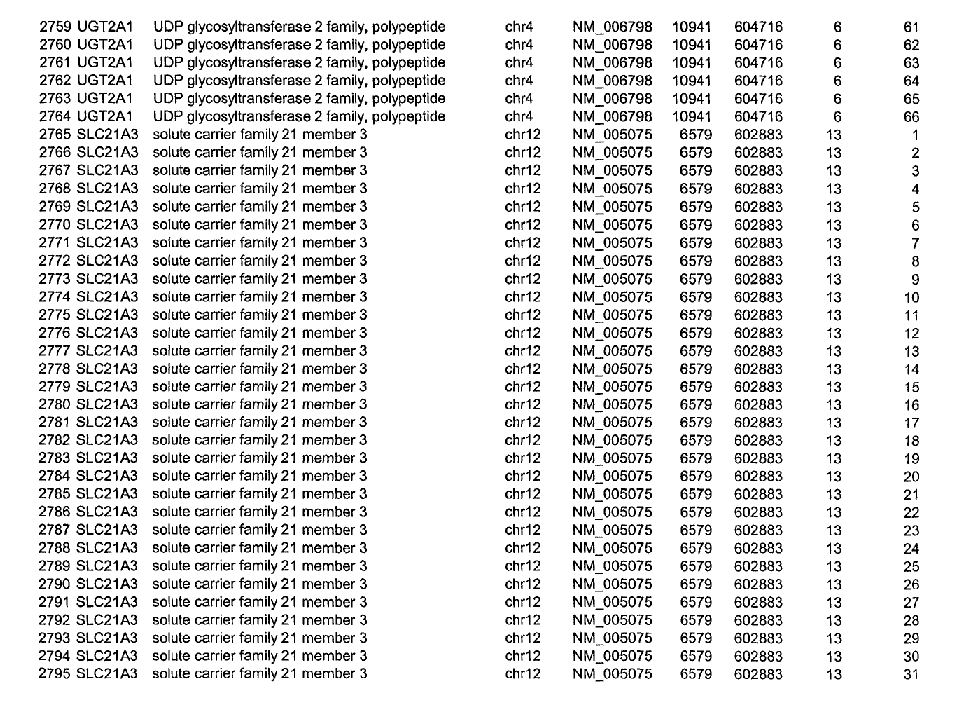

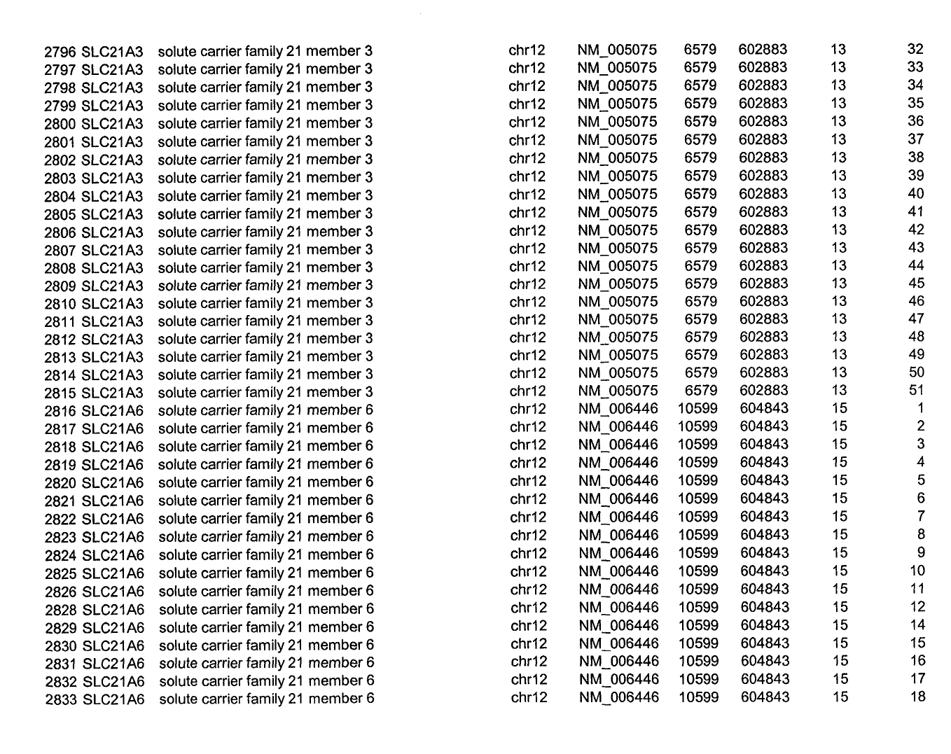

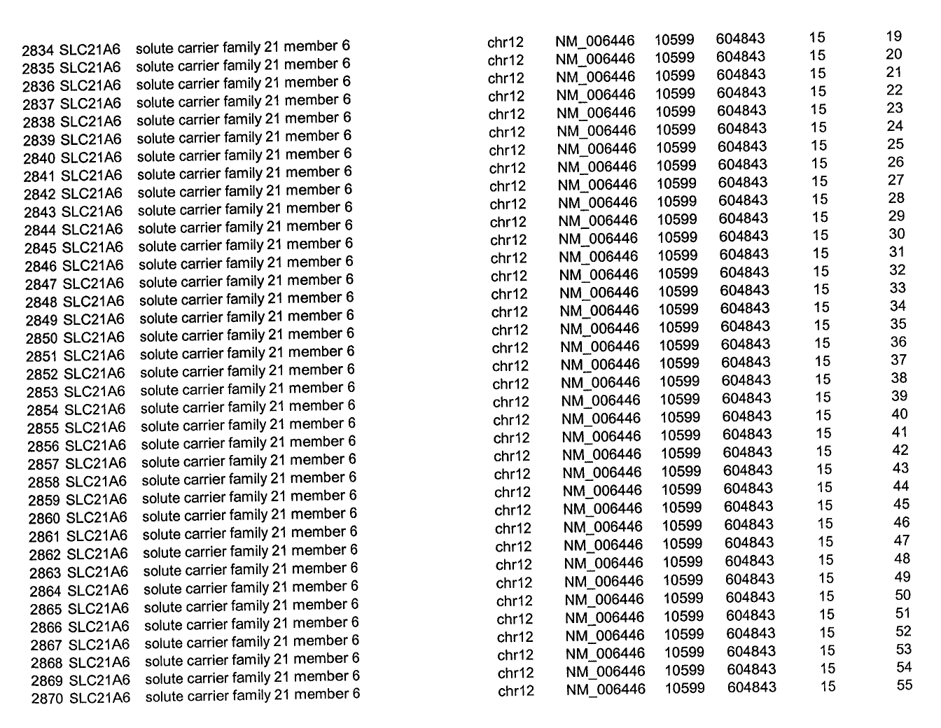

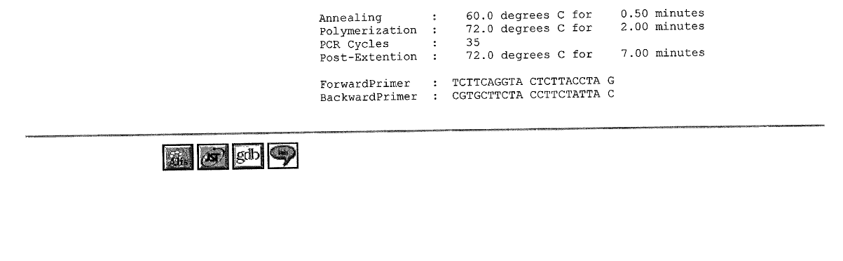

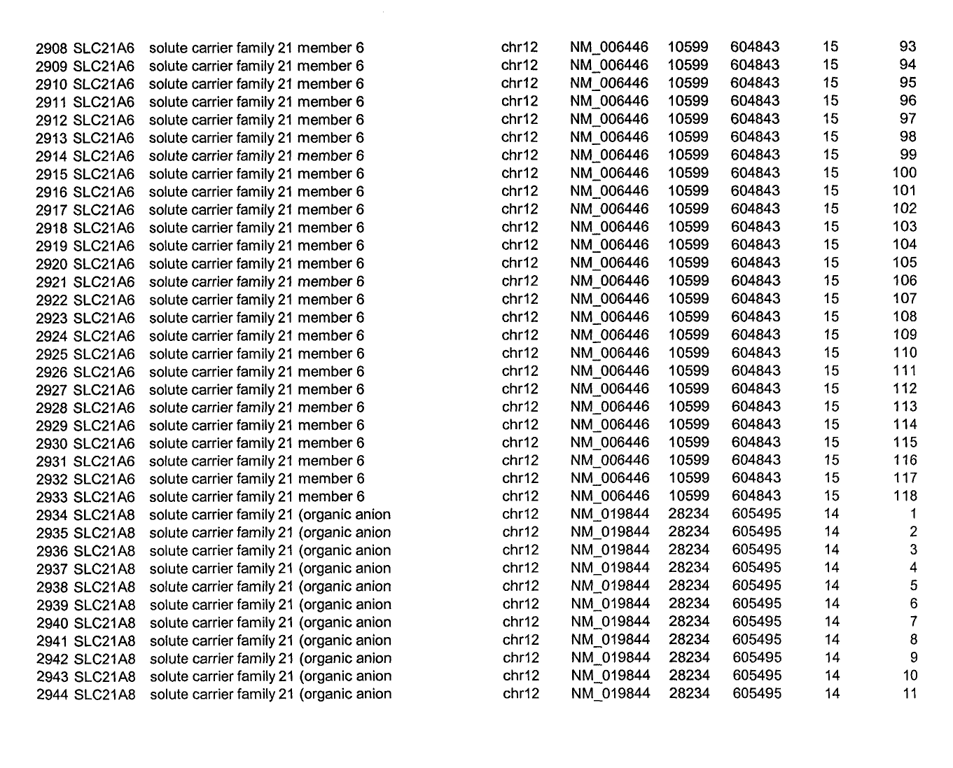

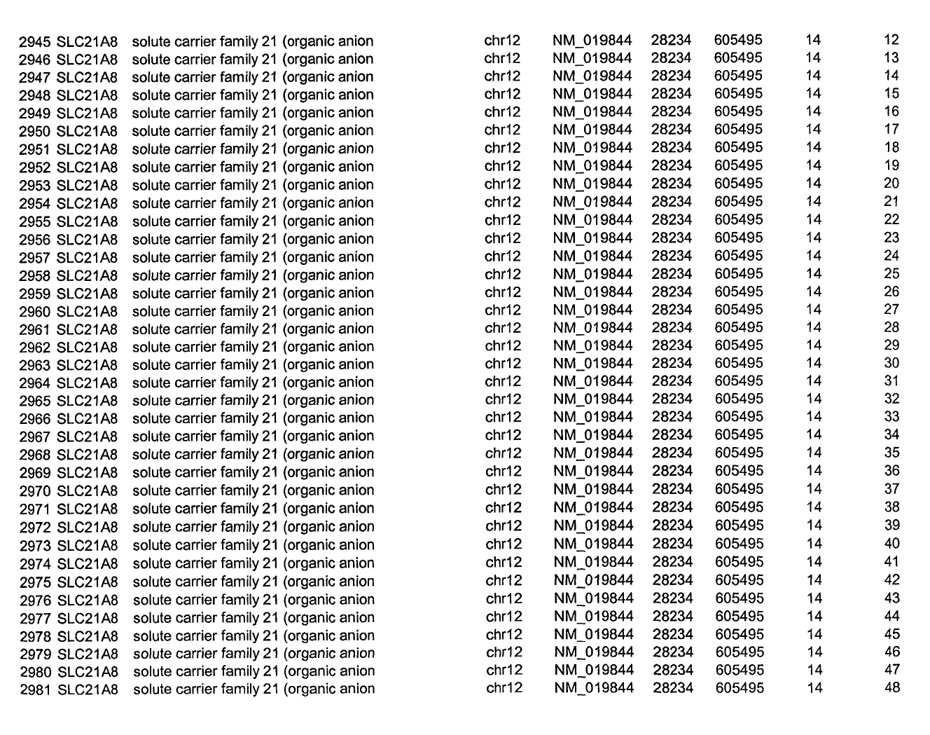

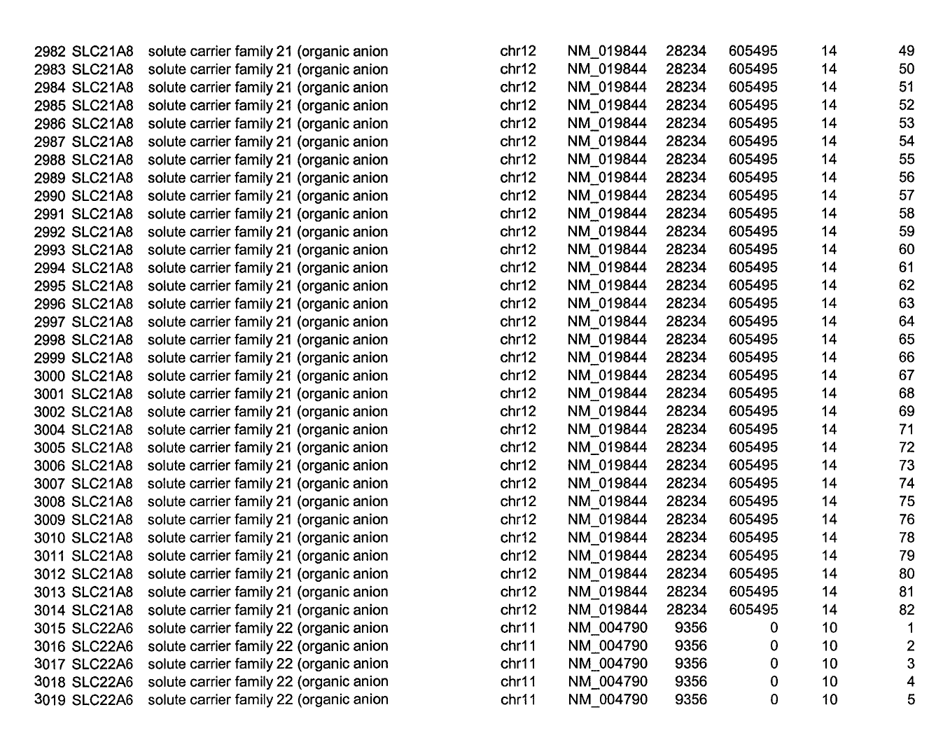

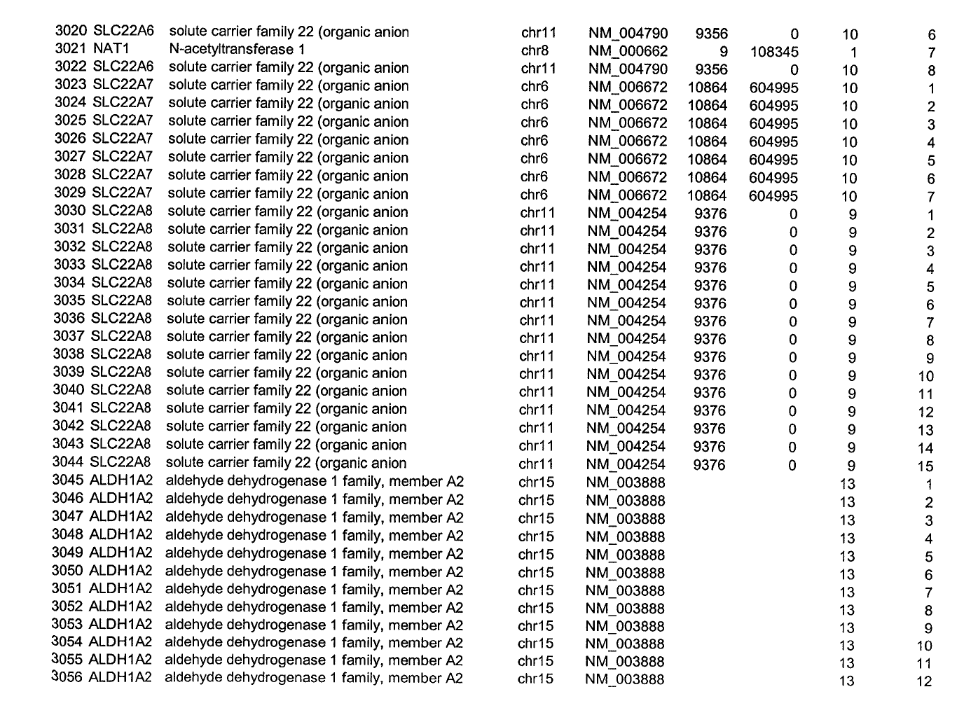

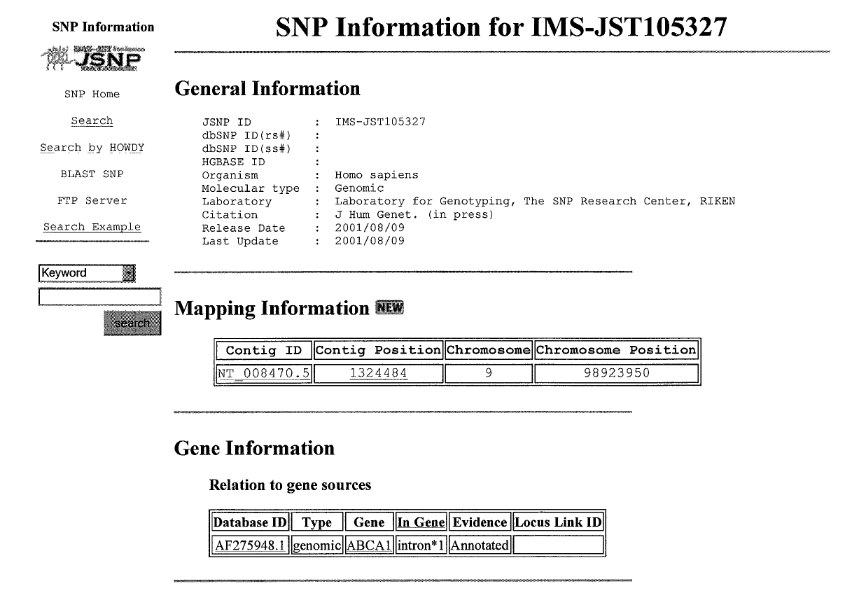

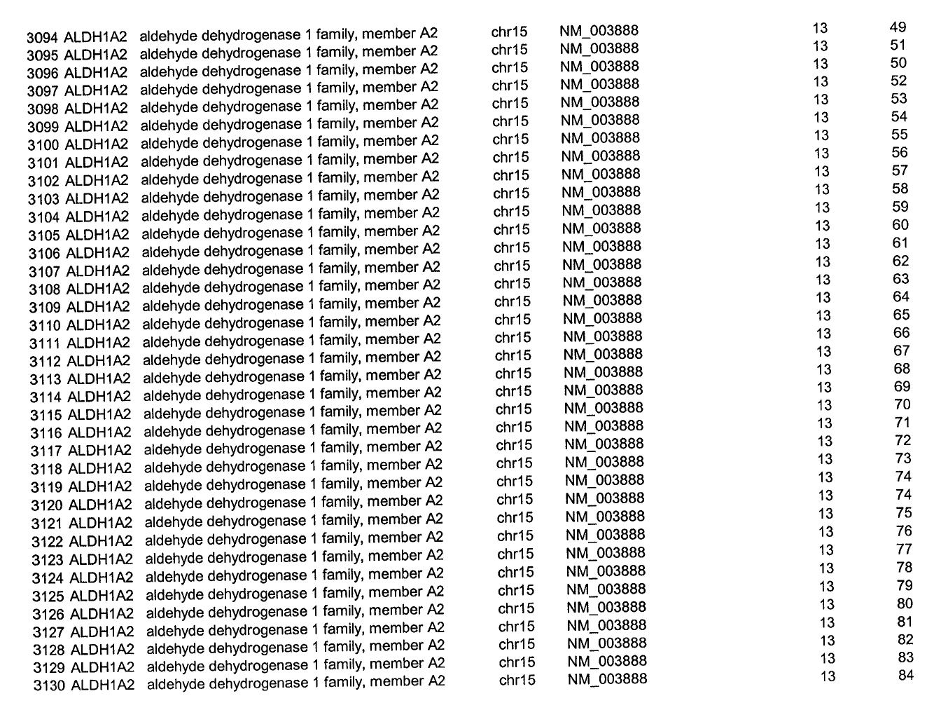

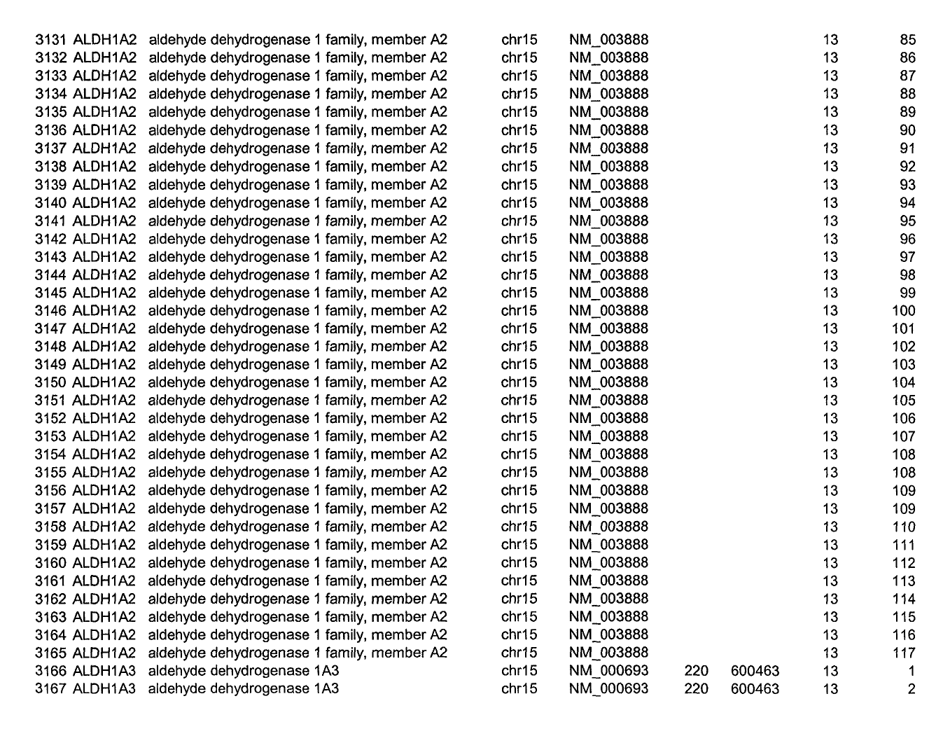

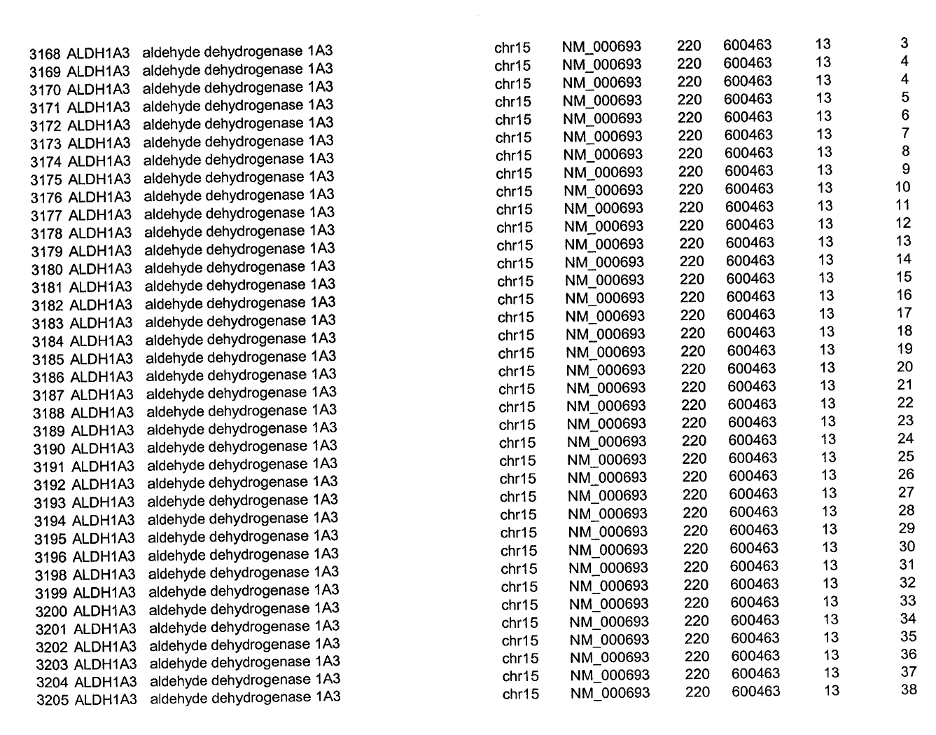

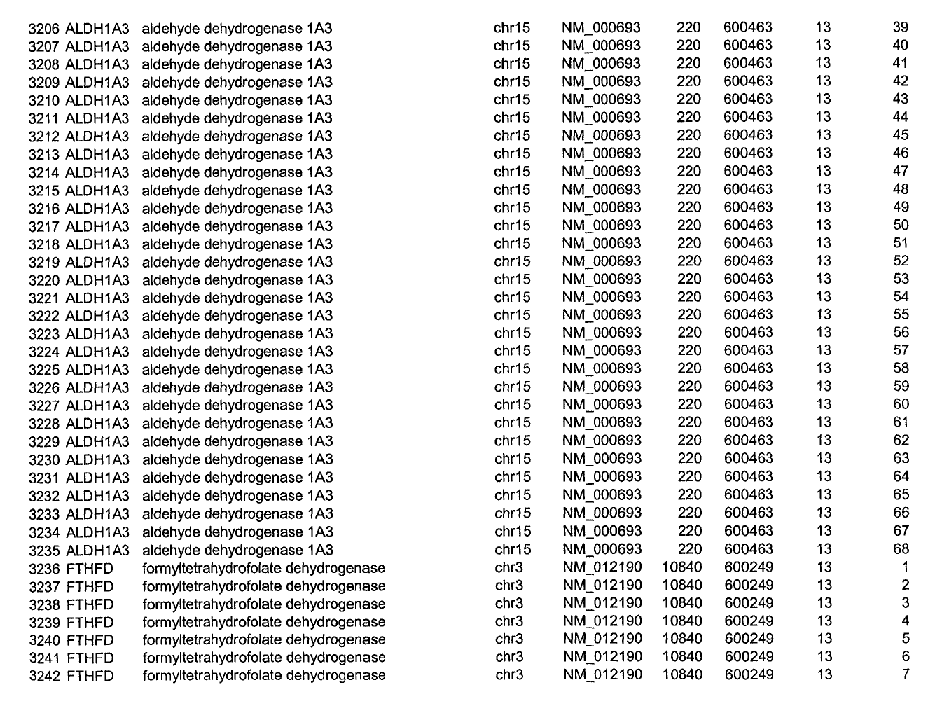

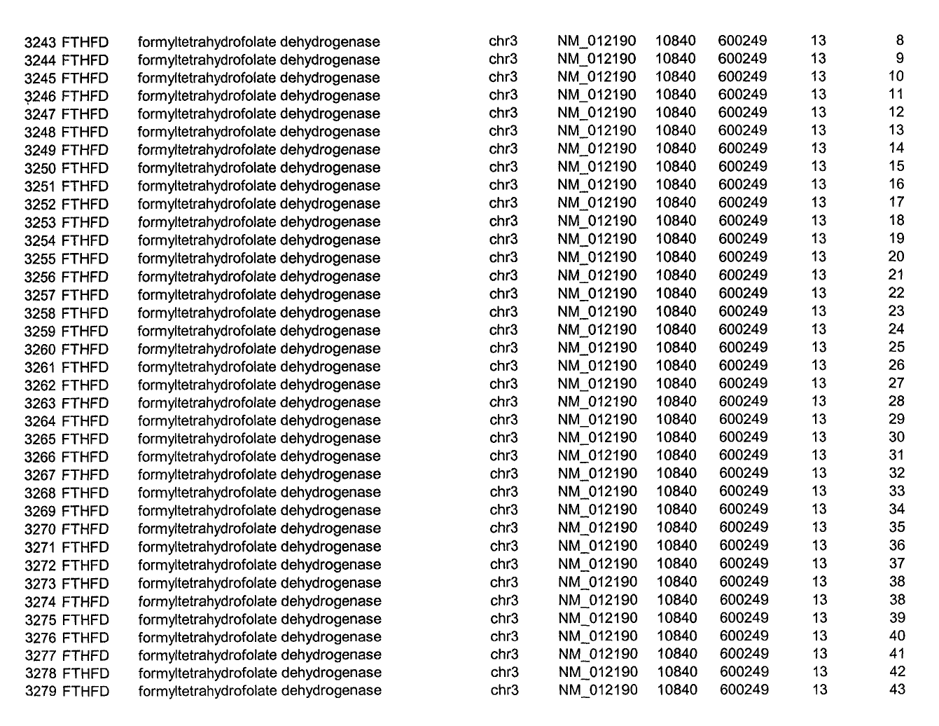

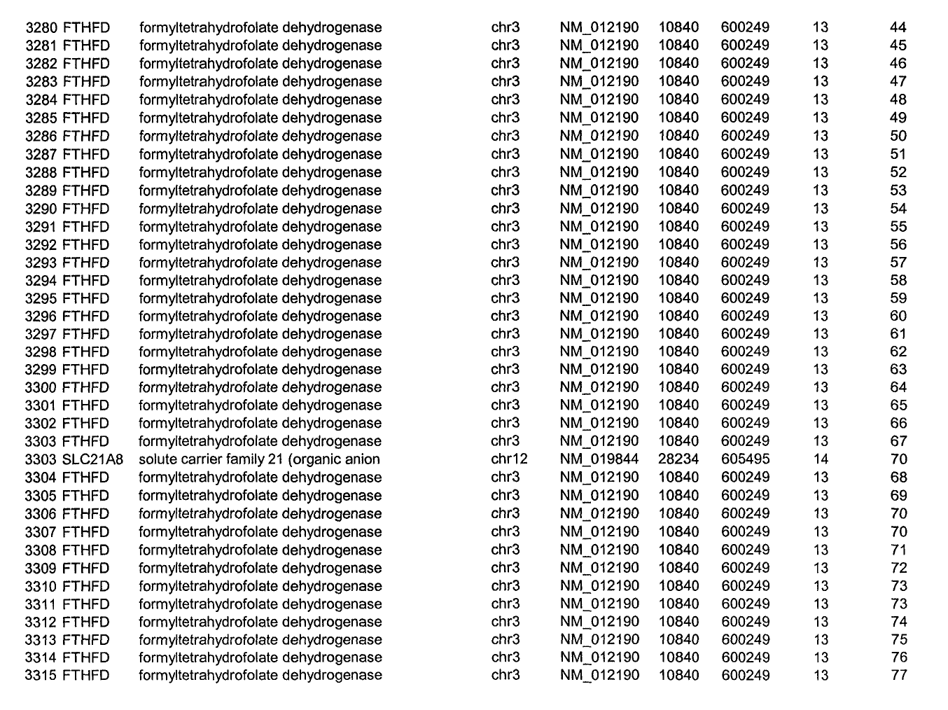

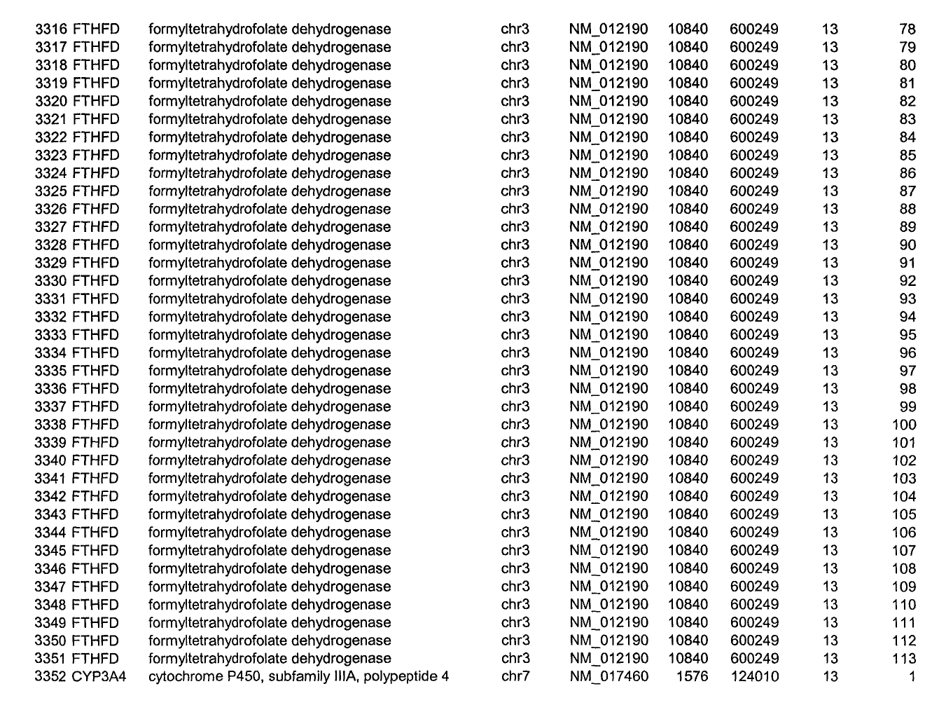

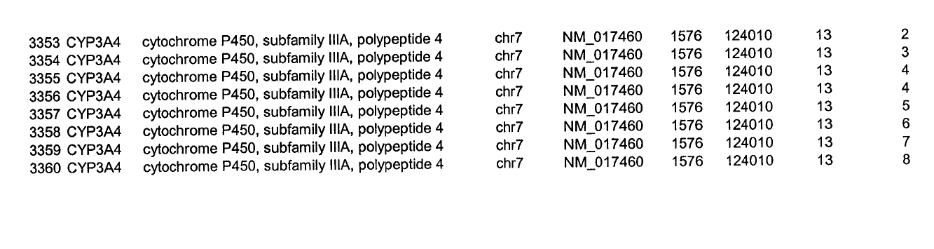

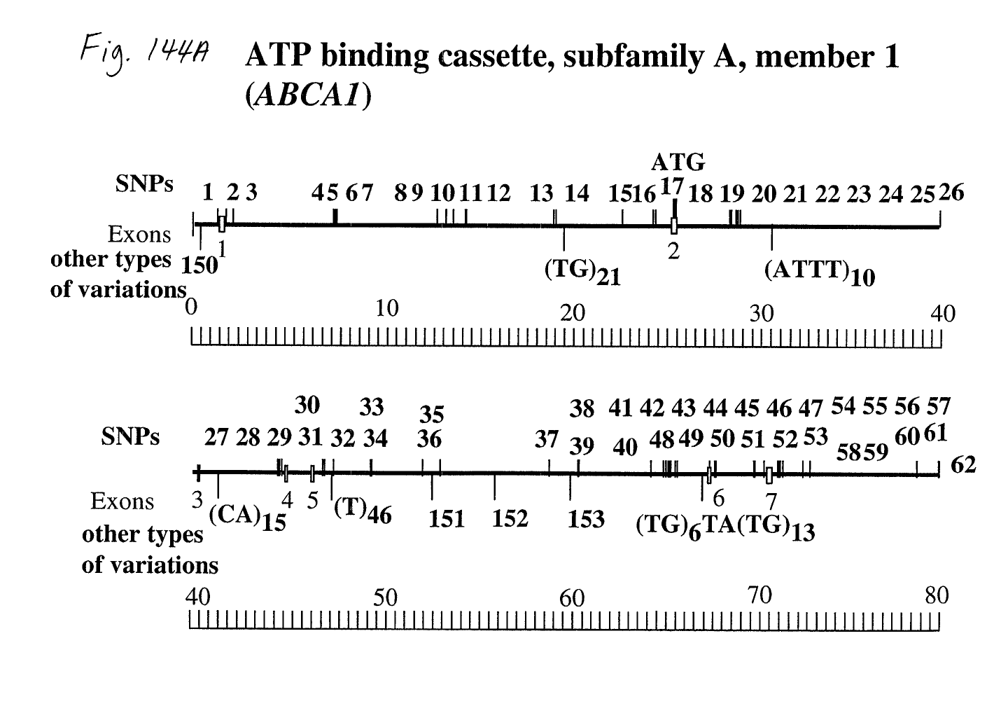

One example of information generated using SEQ ID Nos. 1-3360 and 3361-7669 and information in publicly available databases is provided in FIG. 143. The first column in this figure shows that 3360 entries are made, corresponding to the first 3360 entries found in Table 1. The second column, entitled “GENE” provides a gene name abbreviation, while the next column provides a long gene name. The next columns show the chromosome (CHROM), a reference mRNA accession number (REF.MRNA), a locus link database accession number (L-LINK), an OMIM database accession number (OMIM_ID) which allows disease association information to be readily obtained, the exon count for the gene (EXONS), and the number of polymorphisms in the gene (NO GENE).

Creating an Oligonucleotide Probe or Oligonucleotide Primer

In some embodiments, an oligonucleotide used as a primer and/or probe in the detection method of the present invention serves as the template of the base sequences (Sequence No. 1 through 3360 and 3361 through 7669) shown in Table 1 if, for example, a SNP is to be detected. The primer/probe can be designed so it is synthesized as the base sequence itself or as a portion of the base sequence. In preferred embodiments, the SNP is included in the base sequence of the primer/probe (and denoted in capital letters in the base sequence column of Table 1). The primers/probes may also be complementary to the non-mutant sequence.

The SNP in the following example is designed so it is on the 3′ or 5′ end of the base sequence. It is designed to be within four bases of the 3′ or 5′ end, and ideally within two bases of the end. The SNP can also be in the center of the oligonucleotide base sequence. Here, “center” means the number of the bases from the SNP base to the 5′ end is substantially equal to the number of bases from the SNP base to the 3′ end. If there is an odd number of bases in the oligonucleotide, the central region should be essentially five bases in length, preferably three bases in length, and ideally one base in length. In a base sequence with 41 bases, for example, the central region should be bases 19 through 23, preferably bases 20 through 22, and ideally base 21. If there is an even number of bases, the central region should be four bases and ideally two bases. In a base sequence of 40 bases, for example, the central region should be bases 19 through 22 and ideally base 20.

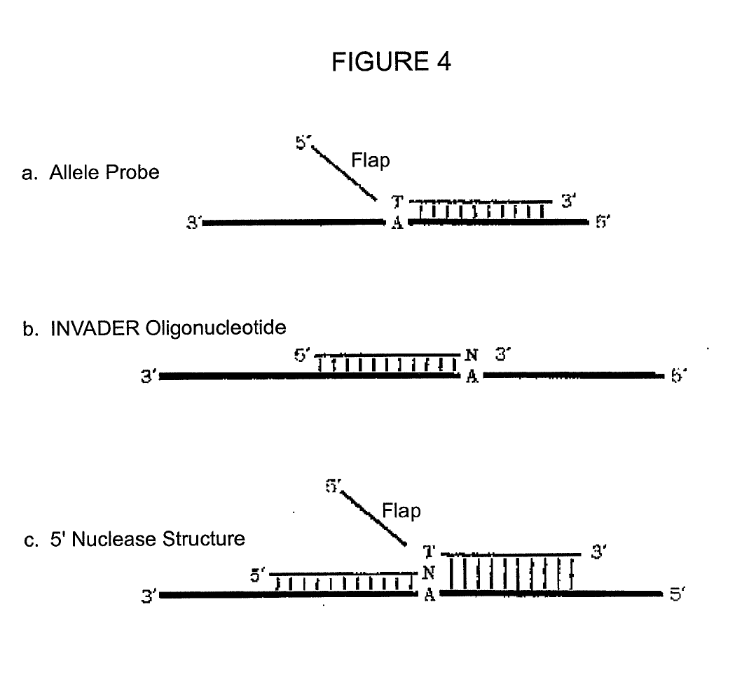

If the polymorphism consists of a plurality of bases, in some embodiments, the probe/primer is designed so the full polymorphism sequence is contained in the probe/primer. In some preferred embodiments, it is designed so one of the bases 1 through 4 on the 5′ end or 3′ end complementing the primer DNA corresponds to the base at the very end of the polymorphism bases. (This is called the “corresponding base”; ideally, it is the base at the 5′ or 3′ end). For example, in the INVADER assay, if a probe and INVADER oligonucleotide are prepared to detect a genetic polymorphism (CAGAGGCT) in No. 12 of NDUFA7 in Table 1 (Sequence No. 828), the position of the corresponding base in the probe in FIG. 4a (a “T” base in the figure) is designed to become “C” at the far left of sequence CAGAGGCT, and the N base in the INVADER oligonucleotide shown in FIG. 4b is designed to replace the “C” at the far left of CAGAGGCT with A, T, C or G). Conversely, if designed so the position of the corresponding base in the INVADER oligonucleotide is the far right “T” in CAGAGGCT, the “N” base is such that the corresponding base in the probe is “T.” Further, the corresponding base of the INVADER oligonucleotide and the allele probe can be set anywhere in the CAGAGGCT sequence.

In preferred embodiments, the length of the base sequence is at least 13 bases, preferably between 13 and 60 bases, more preferably between 15 and 40 bases, and ideally between 18 and 30 bases. These oligonucleotide base sequences can be used as probes, as forward (sense) primers or as reverse (anti-sense) primers to detect target genes.

These oligonucleotides can link regions hybridized with genome DNA in tandem to unhybridized regions. The linking order can be upstream or downstream. The hybridized regions in these oligonucleotides can be designed from base sequence data containing the SNP described in Table 1, and created so the sequence containing the region hybridized with genome DNA closest to the 5′ or 3′ end is the SNP. These oligonucleotides can be used as probes to detect SNP using the INVADER assay.

The primer used in some embodiments of the present invention is designed to determine the functional change caused by the SNPs in the base sequences in Table 1, to determine whether the change is effective or ineffective, and to determine the existence of side effects. It is designed to include the SNP in the PCR-amplified base sequence. In some preferred embodiments, the primer should have at least 15 base sequences, preferably between 15 and 30 base sequences, and ideally between 18 and 24 base sequences. The template DNA regions in the primer base sequence should contain 500 bp or less amplified fragments, preferably between 100 and 300 bp fragments, and ideally between 100 and 150 bp fragments.

The oligonucleotide probes and primers designed in this manner can be synthesized chemically using any method commonly known in the art. For example, the oligonucleotides can be synthesized using a commercially available chemical synthesis device. The production of probes can be conducted automatically by adding fluorescent tags (e.g., FAM, VIC, Cy3) or other labels.

These oligonucleotides can be included in genetic polymorphism detection kits along with polymerase (e.g., Taq polymerase), a buffering solution (e.g., a Tris buffering solution), dNTP, fluorescent dyes (e.g., VIC, FAM), or other desired kit components.

Detection

In some embodiments, the oligonucleotides prepared in the examples above are used as primers/probes, and the genes or a portion thereof (template DNA) encoding the drug metabolizing enzyme is amplified using DNA polymerase. A primer/probe prepared in this manner can be hybridized with template DNA and used to detect DNA with the target genetic polymorphism. The DNA used as the template can be prepared using any method commonly known in the art. Examples include cesium chloride density gradient ultra centrifugation method, the SDS solvency method or the phenol chloroform extraction method.

1 Detection Using PCR

The amplification can be performed using a polymerase chain reaction (PCR). The DNA polymerase can be LA Taq DNA polymerase (Takara), Ex Taq polymerase (Takara), AMPLITAQ Gold polymerase (Applied Biosystems), AMPLITAQ (Applied Biosystems) or Pfu DNA polymerase (Stratagene), as well as other polymerases.

An illustrative example of amplification conditions is provided below. The present invention is not limited to the conditions provided in this example. In preferred embodiments, each cycle in the transforming phase should last between 10 and 40 seconds at 85° C. to 105° C. and preferably 20 and 30 seconds at 94° C., each cycle in the annealing phase should last 30 seconds to 1 minute at 50° C. to 72° C. and preferably 20 seconds to 1 minute at 60° C., and each cycle in the elongation phase should last 1 minute to 4 minutes between 65° C. and 75° C. and preferably 2 minutes to 3 minutes at 72° C. There should be 30 to 40 cycles, although fewer or more cycles are contemplated. In order to completely transform the template DNA and the primer, each cycle in the transforming phase should last 1 minute to 5 minutes at 95° C. before the amplifying cycle. If AMPLITAQ GOLD polymerase manufactured by Applied Biosystems is used, it should last from 8 minutes to 15 minutes and ideally from 10 minutes to 12 minutes. In order to completely elongate the amplified DNA, the elongation phase should last between 1 minute and 10 minutes at 72° C. after the amplification cycle. If the amplified product is not immediately detected, it should be processed again at 4° C. to make sure the amplification was not irregular. In this way, the gene encoding the drug metabolizing enzyme is amplified.

After amplification, gel electrophoresis is performed on the amplified product, the amplified product is stained using ethidium bromide or SYBR Green, and one, two or three bands are detected in the amplified product (DNA fragments) to determine the portion (DNA fragment) of the drug metabolizing enzyme containing the genetic polymorphism in the gene encoding the drug metabolizing enzyme. Polyacrylamide gel electrophoresis or capillary electrophoresis can be performed instead of aerogel electrophoresis. PCR can be performed using a primer tagged with a fluorescent dye to detect the amplified product. A detection method that does not require electrophoresis can also be used, such as bonding the amplified product in solid phase to a microplate and detecting the amplified product using a fluorescent or enzymatic reaction.

2. Detection Using the TAQMAN PCR Method

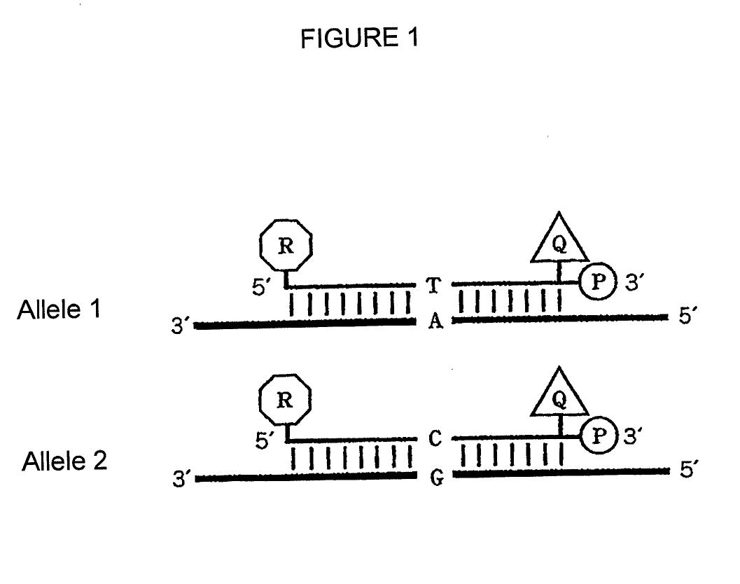

In the TAQMAN PCR method, the PCR reaction is performed using a fluorescent dye-tagged allele-specific oligo and Taq DNA polymerase. The allele-specific oligo used in the TAQMAN PCR method (TAQMAN probe) can be designed based on the SNP data. The 5′ end of the TAQMAN probe is tagged using a fluorescent reporter dye R such as FAM or VIC, and the 3′ end is tagged using a quencher Q (light-quenching substance). (See FIG. 1.). Here, the fluorescent light energy absorbed by the quencher is not detected. Because the 3′ end of the TAQMAN probe is phosphorylated, there is no elongation reaction from the TAQMAN probe in the PCR reaction (FIG. 1). However, a PCR reaction is performed on the TAQMAN probe with TaqDNA polymerase and a primer designed to amplify the region containing the SNP. The following reaction occurs.

First, the TAQMAN probe is hybridized in a specific sequence of template DNA (FIG. 2a) and an elongation reaction is simultaneously performed from the PCR primer (FIG. 2b). Because the Taq DNA polymerase has 5′ nuclease activity, the hybridized TAQMAN probe is severed as the PCR primer elongation reaction continues. When the TAQMAN probe is severed, the quencher has no effect on the fluorescent dye, and the fluorescent light is detected (FIG. 2c).

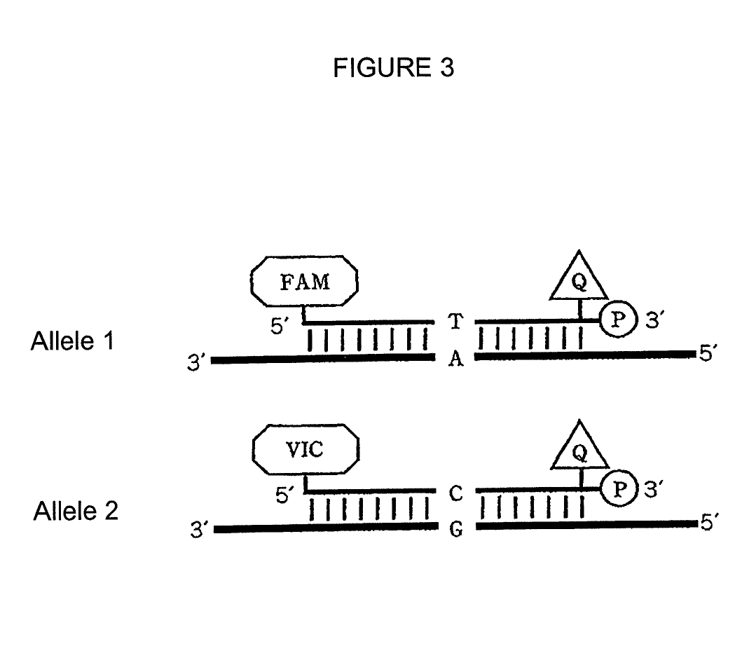

For example, suppose there is an A allele (Allele 1) and a G allele (Allele 2) at the SNP position as shown in FIG. 3. Allele 1 is tagged by a specific TAQMAN probe with FAM and Allele 2 is tagged by a specific TAQMAN probe with VIC (see FIG. 3). Two different allele-specific oligos are added to the PCR drug, and TAQMAN PCR is performed on the detected template. The fluorescence detector then detects the fluorescent intensity of the FAM and VIC. When the SNP position in the allele and the position corresponding to the SNP in the TAQMAN probe are complementary, the probe is hybridized with the allele, the fluorescent dye in the probe is severed by the Taq polymerase, the effect of the quencher is eliminated, and the intensity of the fluorescence is detected.

If the template is homozygous for Allele 1, strong FAM fluorescence is detected and hardly any VIC fluorescence is detected. If the template is heterozygous for Allele 1 and Allele 2, both FAM and VIC fluorescence are detected.

3. SNP Detection Using the INVADER Assay

In the INVADER assay, an allele-specific oligo and the template are hydridized to detect the SNP. In the INVADER assay, two different non-tagged oligos and one fluorescent dye-tagged oligo are used. One of the two non-tagged oligos is known as the probe. In some embodiments, the probe has a. region hybridized to the genome DNA (template DNA) and a region (called a flap) that is not hydridized with the genome DNA, and that has a sequence unrelated to the sequence of the genomic DNA. The hybridized region has base sequences corresponding to the SNP (FIG. 4a). The flap sequence is complementary to a FRET probe (described below). The other of the two non-tagged oligos is called the INVADER oligonucleotide. This oligonucleotide is designed so that it is hybridized in complementary fashion from the SNP position towards the 3′ end of the genome DNA (FIG. 4b). In some preferred embodiments, the sequence corresponding to the SNP position can be any base (denoted by N in FIG. 4b). When the template DNA genome is hybridized with the two probes, the base (N) from the INVADER oligonucleotide is inserted in the SNP position (FIG. 4c) forming a cleavage structure at the SNP position.

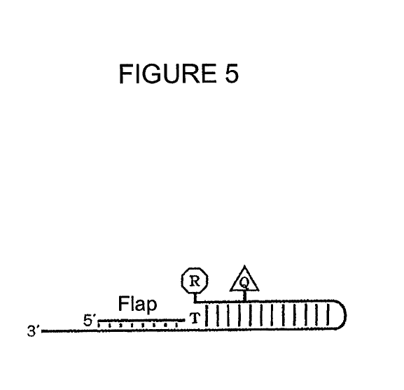

In some embodiments, the fluorescent dye-tagged oligonucleotide is a sequence completely unrelated to the alleles. This probe is a FRET (fluorescence resonance energy transfer) probe (FIG. 5). The fluorescent dye R tags the base (reporter) at the 5′ end of the FRET prove. A quencher Q absorbs the fluorescence. Here, the quencher absorbs the fluorescent light and the light is not detected. A specific region (Region 1) is designed on the 5′ end of the FRET probe (reporter base) to face the 3′ end from Region 1 (This region is Region 2). As a result, Region 1 and Region 2 form a complementary duplex (FIG. 5). The 3′-region from the regions forming the complementary duplex can be hybridized with the flap of the allele probe to form a complementary chain (FIG. 5).

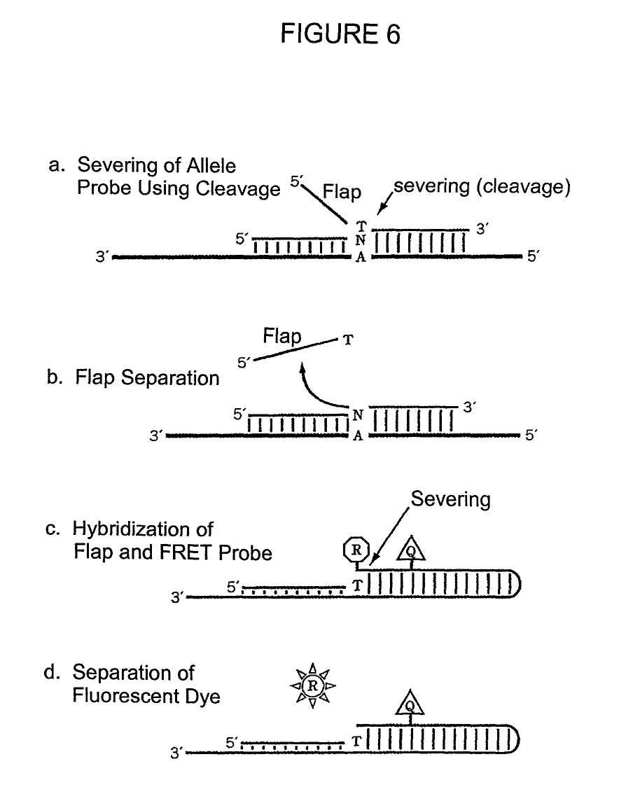

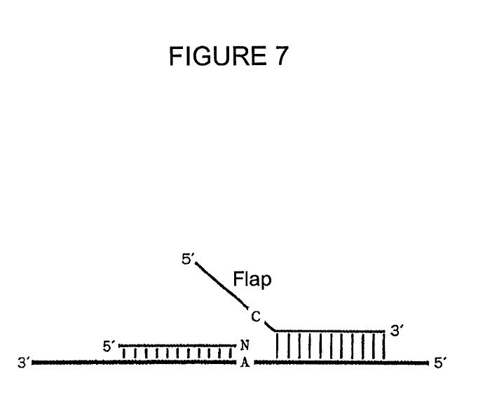

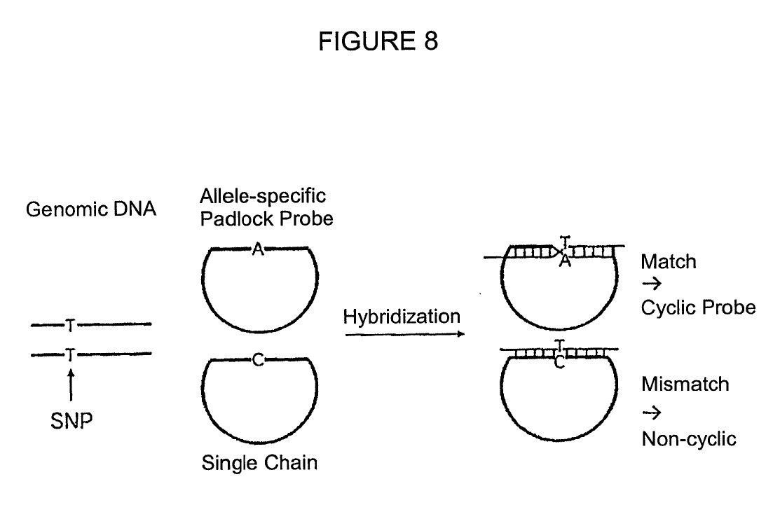

In the INVADER assay, a cleavage agent (e.g., CLEAVASE enzyme, Third Wave Technologies, Madison, Wis.) is used, which is an enzyme (5′ nuclease) with specific endonuclease activity for identifying and cleaving a specific DNA structure. When the genome DNA, the probe and the INVADER oligonucleotide form a cleavage structure at the SNP position, the cleavage agent severs 3′ of the SNP position on the allele probe. The section with three bases forming a flap with the 5′ end is identified as shown in FIG. 4c, and the flap is severed. The structure with the SNP position is identified by the cleavage agent (FIG. 6a), the probe is severed at the flap position, and the flap is separated (FIG. 6b). Next, the released flap from the probe bonds with the FRET probe in complementary fashion to form a duplex (FIG. 6c). The cleavage agent identifies this structure and cleaves the section with the fluorescent dye. The cleaved fluorescent dye is no longer affected by the quencher and fluorescent light becomes detectable (FIG. 6d). If the SNP position does not match the sequence corresponding to the SNP in the allele probe as shown in FIG. 7, the specific DNA structure is not identified by the cleavage agent, the probe is not severed, and fluorescent light is not detected.

When the SNP is T/C, for example, a T INVADER oligonucleotide, a T probe, a FRET probe with FAM bonded to the reporter for the T SNP, a C INVADER oligonucleotide, a C probe and a FRET probe with VIC bonded to the reporter for the C SNP are prepared. These are combined and SNP detection is performed. If there is a T/T homo, FAM fluorescence is generated. If there is a C/C homo, VIC fluorescence is detected. If there is a T/C hetero, both FAM and VIC fluorescence are detected. Because the FAM and VIC fluorescence wavelengths are different, both can be readily identified.

Detection Using the SniPer Method

In order to detect SNP using the SniPer method, an allele identifier is amplified using RCA. The genome DNA template is a straight chain, and a probe is hybridized with the genome DNA. When there is a complementary match between the probe sequence and the genome DNA template sequence and a complementary chain forms, a ligation reaction on the genome DNA forms a ring. As a result, RCA continues on cyclic DNA. If the end of the probe does not match the genome DNA, the RCA reaction does not occur because there is no ligation and no ring. In the SniPer method, therefore, a single chain probe is designed to anneal the genome DNA and create a ring. This single chain probe is called a padlock probe. The severed end of the padlock probe is the sequence corresponding to the target SNP. The padlock probe and the genome DNA mix and a ligation reaction occurs. If the severed end of the padlock probe and the SNP section of the genome DNA are complementary, the severed end of the padlock probe connects and forms a ring during the ligation reaction. If they are not complementary, a ring does not form. Therefore, only a padlock probe corresponding to the target SNP forms a ring and is amplified by the DNA polymerase. The presence of amplification is used to detect the SNP. A synthetic oligonucleotide with a hairpin structure and a fluorescent dye and quencher on both ends can be used in the detection process.

Detection Using the MALDI-TOF/MS Method

In the matrix assisted laser desorption-time of flight/mass spectroscopy (MALDI-TOF/MS) method, SNP typing is performed using a mass spectrometer. A preferred embodiments of this method has the following steps.

(i) PCR Amplification and Refinement of DNA Fragments Containing SNP

After making sure the base at the SNP location and the PCR primer do not overlap, the DNA fragment is amplified, exonuclease or alkali phosphatase processing is performed on the amplified product, the dNTP is removed, and the amplified fragment is refined.

(ii) Primer Extension Reaction (Thermal Cycle) and Refinement

A primer ten or more times the template in the region identified as the PCR product is added, a thermal cycle reaction is performed, and a primer elongation reaction is performed. The primer used here is designed so the 3′ end is next to the base corresponding to the SNP position. The primer length should be 15 to 30 bases, ideally 20 to 25 bases. If there is a multiplex reaction, a sequence that is not complementary to the template is added to the 5′ end. There should be 20 to 30 (ideally 25) thermal cycles at two different temperatures. These should be 85 to 105° C. (ideally 94° C.) and 35 to 40° C. (ideally 37° C.).

The reaction product is then refined using a refining kit so it can be used in the mass spectrometer.

(iii) Mass Spectroscopy Using a Mass Spectrometer

The elongated and refined reaction product is applied to the mass spectrometer, and a quality of the target product is measured. In other words, the refined product is mixed with the matrix and 0.5 to 1.0 mL spots are formed on the MALDI plate. After drying the plate, the substance is irradiated by a laser beam and a spectrogram is produced.

Detection Using the Base Sequence Determining Method

In the present invention, a polymorphism can be detected using an elongation reaction on a single base. In other words, four different types of dideoxynucleotides identified by different fluorescent compounds are added to reaction systems including the gene to be detected and a single base elongation reaction is performed. Here, the base to be elongated is the polymorphism. Two reactions are performed; one to stop the DNA synthesis and another to identify the 3′ end of the DNA molecule with fluorescence. Electrophoresis is performed on four different reaction solutions with the same lanes and capillaries for the sequencing gel. The sequence is determined by detecting the differences in the fluorescent dyes identifying the DNA bands using a fluorescence detector. The oligonucleotides with one base elongated have the elongation confirmed using different types of fluorescent dyes in a fluorescence detector and mass spectrometer. Instead of fluorescent-tagged dideoxynucleotides, the primer can be identified using fluorescence used with non-tagged dideoxynucleotides.

Drug Evaluation

Using information obtained by the methods of the present invention, the efficacy and stability of the drug metabolized by the drug metabolizing enzyme can be evaluated.

For example, in some embodiments, the drug can be evaluated using a typing system. In other words, the frequency of expressed and unexpressed alleles (e.g., toxic alleles that cause undesired side effects) can be compared using any one of the detection methods mentioned above. Once they have been compared, markers can be selected to indicate, for example, a toxic expression where the allele frequency differs. In statistical analysis, this is usually set as ×2. However, this is different in other methods such as the Fisher method. The active components (altered and metabolized drug components) in the drug will be reflected in blood and tissue concentrations. All of the genetic polymorphisms can be checked against the causes of the toxic effects to isolate specific correlating genetic polymorphisms. The substances corresponding to the probes or primers used to analyze all of the genetic polymorphisms are prepared beforehand on reaction plates, cards or glass plates, and unprepared human genome DNA is added and reacted to determine the allele pattern. If there are genetic polymorphisms correlating with toxicity or other phenotypes, then human side-effects can be expected or predicted. The same is true of drug effectiveness. Because the genetic polymorphisms correlating to effectiveness and side-effects differ depending on the drug, typing performed using genetic polymorphisms can be performed to anticipate effectiveness and side-effects.

Differences in allele frequency can be determined in certain instances by comparing the frequency of genetic polymorphisms to effectiveness/ineffectiveness or the presence/absence of side-effects. If, for example, an SNP analysis is performed on persons with a toxic reaction (side-effect) to Drug A, the results may show a 90% of the people have T/T (e.g., detected based on the intensity of fluorescent FAM light). The same results may show 10% of people with no toxic reaction have a T/T and 90% have a C/C. As a result of the SNP analysis, the evaluation may be not to administer Drug A to persons with T/T.

Drug Screening

In the present invention, the genetic polymorphism data obtained as described above is compared to genetic polymorphism data from genes encoding certain drug metabolizing enzymes to indicate the safety and effectiveness of drugs metabolized by these drug metabolizing enzymes. Therefore, the genetic polymorphism data obtained using the method of the present invention can be used to determine the likely effectiveness of certain drug therapies and to select the appropriate drug.

The evaluation methods described above can be used. Genetic polymorphisms with correlations to side-effects and effectiveness are said to be influenced by the activation, transfer and translation of certain enzymes. The cause and effect relationship with the side-effect or effectiveness expression mechanism may be indirect. The metabolization of drugs is being studied by pharmaceutical companies in laboratory and clinical testing. If there are genetic polymorphisms in enzyme genes correlating with severe side-effects, they can be removed and used under different conditions. The same is true of effectiveness. Drugs can be screened, therefore, using side-effects and effectiveness data. A wide variety of conditions and diseases (See e.g., Physician's Desk Reference) benefit from analysis using the systems and methods of the present invention.

In some embodiments of the present invention, a sample is taken from a subject (e.g., by a drug company) and sent to a laboratory for analysis using a detection assay. The laboratory results (e.g., detection assay test result data) is returned to the party providing the sample such that an appropriate decision can be made, including, but not limited to, development or administration of a drug to a subject.

In clinical testing (Tests I through III), the frequency of the expression of genetic polymorphisms can be studied in volunteers exhibiting certain side-effects and volunteers not exhibiting the same side-effects to a drug. In this way, novel genetic polymorphisms correlating with side-effects and effectiveness can be detected. This information can be used to screen drugs. Exemplary drugs and drug-related data and other information that find use in or with the present invention, including but are not limited to the methods and databases described herein, are described in the PHYSICIANS' DESK REFERENCE (PDR). (e.g., 2002 Edition, Medical Economics Company, Inc., Montvale, N.J.). The PDR is expressly incorporated by reference herein as if fully set forth.

### EXPERIMENTAL EXAMPLES

The following examples are provided in order to demonstrate and further illustrate certain preferred embodiments and aspects of the present invention and are not to be construed as limiting the scope thereof.

### Example 1

### Obtaining SNP Data

(1) DNA Extraction

Blood was extracted from 48 unrelated people in the presence of EDTA. The DNA was extracted in the following way based on the method in the Genome Analysis Manual (Yusuke Nakamura ed., Springer-Verlag Tokyo).

Ten milliliters of blood was transferred to a 50 ml test tube and centrifuged for five minutes at room temperature and 3000 rpm. After the supernatant (blood serum) had been removed using a pipette, 30 ml of RBC-dissolving buffer (10 mM NH4 HCO3, 144 mM NH3C1) was added. After mixing until there was no sediment, it was allowed to stand for 20 minutes at room temperature. After being centrifuged for five minutes at room temperature and 3000 rpm, the supernatant (blood serum) was again removed using a pipette to obtain white blood cells. Another 30 ml of RBC-dissolving buffer was added and the process was repeated twice. Then, 4 ml of proteinase K buffer [50 mM Tris-HCl (pH 7.4), 100 mM NaCl, 1 mM EDTA (pH 8.0)] was added to the white blood cells, 200 ml of SDS was added, 200 ml of 10 mg/ml proteinase K was added, and the solution was tumble-mixed. The solution was then allowed to stand overnight at 37° C. The next day, 4 ml of phenol was added, and the solution was slowly tumble-mixed for four hours using a Taitec T-50 Rotator. After being centrifuged for 10 minutes at room temperature and 3000 rpm, the supernatant was removed using a new tube. Then, 4 ml of phenol-chloroform-isoamylalcohol (volume ratio 25:24:1) was added, the solution was tumble-mixed for two hours in the manner described above, and the solution was centrifuged. The supernatant was removed using a new tube, 4 ml of chloroform-isoamylalcohol (volume ratio 24:1) was added, and the solution was tumble-mixed. Fibrous white precipitate (DNA) was removed using a 2 ml tube, 1 ml of 70% ethanol was added, and the solution was tumble-mixed. The DNA was transferred to a new tube, dried and dissolved in 500 ml of TE solution [10 mM Tris-HCl (pH 4.7), 1 mM EDTA (pH 7.4)] to obtain a genome DNA sample.

(2) PCR

A genome sequence was obtained from the GenBank DNA Database. After removing the repeating sequences using the RepMask computer program, the PCR primer was set so there would be approximately 1 kb of PCR product. The genome DNA from 48 unrelated people was prepared at the same concentration. After mixing the same amount of DNA from three people in a single tube, 60 ng was used in the PCR. The PCR was Ex-Taq (Takara 2.5 U) and performed using the GeneAmp PCR System 9700 (PE Applied Biosystems). After reacting for two hours at 94° C., denaturing was performed for 30 seconds at 96° C., annealing was performed for 30 seconds at 55° C. or 60° C., and elongation was performed for one minute at 72° C. in each cycle. There were 35 cycles.

(3) Sequence

After refining the PCR product using Arraylt (Telechem), the sequence reaction was performed using the BigDye Terminator RR Mix (PE Applied Biosystems). After reacting for two hours at 96° C., denaturing was performed for 20 seconds at 96° C., annealing was performed for 30 seconds at 50° C., and elongation was performed for 4 minutes at 60° C. in each cycle using the GeneAmp PCR System 9700 (PE Applied Biosysytems). There were 25 cycles. After the sequencing reaction, the sequencing was analyzed using the ABI Prism 3700 DNA Analyzer.

(4) SNP Detection

An analysis was performed on the SNP detection using the PolyPhred computer program (Nickerson et al., 1997, Nucleic Acid Res., 25, 2745-2751).

(5) Results

The SNP results shown in Table 1 were obtained. The analyzed drug metabolizing enzyme, the abbreviation of the enzyme, the databank (GenBank) accession number, the structure of the gene for the drug metabolizing enzyme, and the position of the SNPs are shown in FIG. 9 through FIG. 141 and FIG. 144 through 312. In FIG. 9 through FIG. 141 and FIG. 144 through 312, the exons are blank boxes or black lines in the genes denoted by the horizontal lines. The position of the SNPs is denoted above the genes with solid lines and numbers.

### Example 2

### Typing

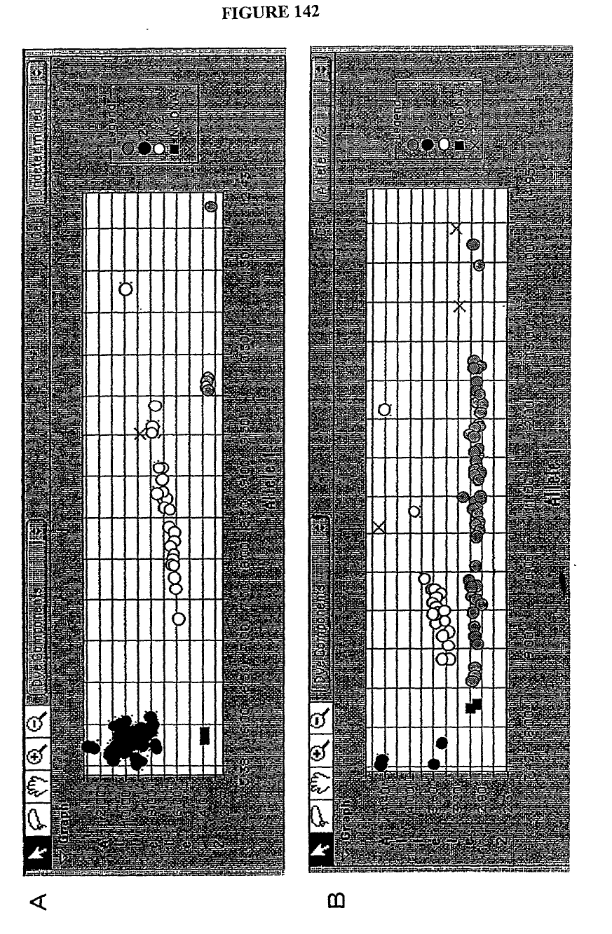

Typing was performed on two different groups of patients using the INVADER assay. In FIG. 142, the x-axis (Allele 1) indicates the intensity of the FAM fluorescent light corresponding to T, and the x-axis (Allele 2) indicates the intensity of the VIC fluorescent light corresponding to C. The slanted line indicates the SNP pattern for T/T, the black circles denote the pattern for C/C, and the white circles denote the pattern for T/C. The black squares indicate the background values. The x marks indicate where the detection failed. The group of patients in the graph for panel A (top) had many C/C SNP patterns and the group of patients in the graph for panel B (bottom) had many T/T SNP patterns.

### Example 3

### SNP Detection

Genome DNA was extracted from five unrelated people using the method described in Example 1, and the SNPs in three different drug metabolizing enzyme genes (EPHX1, ABCB2, AANAT) were detected using the INVADER assay method. The INVADER oligonucleotides and probes were designed using base sequence No. 3 (Sequence No. 49) and No. 17 (Sequence No. 63) in the case of EPHX1, base sequence No. 4 (Sequence No. 4) and No. 11 (Sequence No. 11) in the case of ABCB2, and base sequence No. 3 (Sequence No. 561) in the case of AANAT. The positions of the SNPs are shown in Table 1.

The results are shown in Table 2.

TABLE 2

| | EPHX1 | | ABCB2 | | AANAT |
|---|---|---|---|---|---|
| Drug | No. 3 | No. 17 | No. 4 | No. 11 | No. 3 |
| Metabolizing | Seq. | Seq. | Seq. | Seq. | Seq. |
| Enzyme Gene | No. 49 | No. 63 | No. 4 | No. 11 | No. 561 |
| SNP | (T/G) | (A/G) | (G/T) | (G/A) | (T/A) |
| Subject I | T/T | A/G | T/T | G/A | T/T |
| Subject II | T/T | A/A | G/G | G/G | T/A |
| Subject III | T/G | A/A | G/G | A/A | T/T |
| Subject IV | G/G | A/G | G/T | G/G | T/T |
| Subject V | T/G | A/G | G/T | G/A | T/A |

As shown in Table 2, the SNPs in the drug metabolizing genes of patients can be detected and the patterns determined using the method of the present invention.

### Example 4

### Correlation Between SNP Genotypes and Optimal Amounts of a Medicament for Treatment Validity and Safety

In this example, validity and safety of medicaments were investigated using SNP analysis.

Thiopurine S-methyltransferase (TPMT) is an enzyme that transfers a methyl group to a sulfur atom attached to a purine ring, and is one of the major enzymes for metabolizing drugs such as the anti-cancer agents 6-mercaptopurine and 6-thioguanine, and thiopurine derivatives such as the immunosuppressive agent azathioprine. This example shows a correlation between optimal amounts of azathioprine and various combinations of the alleles at the 868^{th} SNP of intron 3 of TPMT (Seki, et al., J Hum Genet 45(5):299 [2000], incorporated by reference herein in its entirety; Accession No. AB045146.1) (G or T alleles) and the 2682^{nd} SNP of intron 3 (C or A alleles) (Table 3 and Table 4).

TABLE 3

| 868 | 2682 | High | Low |
|---|---|---|---|
| TT | AA | 2 | 0 |
| TT | AT | 3 | 0 |
| TT | TT | 1 | 0 |
| GT | AA | 0 | 2 |
| GT | AT | 1 | 7 |
| GT | TT | 4 | 1 |
| GG | AA | 1 | 0 |
| GG | AT | 0 | 1 |
| GG | TT | 1 | 0 |

Optimal amounts of azathioprine were determined by adopting suppression of rejection after renal transplantation as an index. A group of patients in which the validity of treatment with 100 mg/day of azathioprine was confirmed was designated as a high dose group, and a group of patients in which side effects developed with treatment of 100 mg/day, but in which validity was confirmed with a treatment of 50 mg/day was designated as a low dose group. Table 3 indicates the number of patients having each combination of alleles, with the columns labeled “High” and “Low” representing the numbers of patients of each genotype in the high dose and the low dose groups, respectively. Side effects include leukopenia, anthema, angiitis, nausea/vomiting, anorexia, diarrhea, malaise, myalgia, arthralgia, fever, chill, and dizziness. More serious side effects include, for example, blood disorders, shock-like symptoms, infectious diseases, and hepatic disorders, and renal disorders.

Investigation of a correlation between the high dose and low dose groups and the two types of SNPs indicated above revealed that when G is present in at least one allele at the 868^{th} SNP of intron 3 (G/G homozygous or G/T heterozygous) and A is present in at least one allele at the 2682^{nd} SNP of intron 3 (A/A homozygous or A/T heterozygous), side effects were developed with 100 mg/day and 50 mg/day was an optimal amount for 10 out of 12 patients (low dose group), while 100 mg/day was an optimal amount for 11 out of 12 patients with other allele combinations (high dose group) (Table 4). Investigation of this combination of two SNP loci in patients enables prediction of optimal amounts of azathioprine for treatment prior to the administration of the drug, for improved validity and safety. These results indicate that the validity and safety of medicaments can be predicted using analysis of SNPs associated with medicament metabolic enzymes, e.g., as described in this specification and including but not limited to the DME-associated SNPs listed in Table 1. As used in this example only, the term “optimal amount” refers to the best dosage selected from the tested amounts of 50 mg/day or 100/mg per day. It will be appreciated by those skilled in the art that a study testing additional amounts of a medicament (e.g., a study in which amounts are varied in smaller increments, such as 40, 50, 60, 70, 80, 90, etc. mg/day) would provide additional information regarding ranges of amounts giving optimal performance for patients having a particular genotype, and that optimal amounts of this or any other medicament are not limited to the particular amounts of 50 or 100 mg/day tested in this example.

TABLE 4

Optimal amount

| Genotype | 100 mg/day | 50 mg/day |
|---|---|---|
| G as the 868^{th} SNP and A | 2 | 10 |
| as the 2682^{nd} | | |
| Other combinations | 11 | 1 |

(Fisher exact test: p = 0.0003)

### Sequence Listing Free Text

SEQ ID NO:39: n indicates t (Position 21).

SEQ ID NO:64: n indicates c (Position 21).

SEQ ID NO:580: n indicates a or deletion (Position 21).

SEQ ID NO:634: n indicates a or deletion (Position 21).

SEQ ID NO:656: n indicates a or deletion (Position 21).

SEQ ID NO:658: n indicates c or deletion (Position 21).

SEQ ID NO:671: n indicates a or deletion (Position 21).

SEQ ID NO:672: n indicates g or deletion (Position 21).

SEQ ID NO:673: n indicates c or deletion (Position 21).

SEQ ID NO:674: n indicates (cctgy)x or deletion (Position 21).

SEQ ID NO:676: n indicates gaa or deletion (Position 21).

SEQ ID NO:677: n indicates ag or deletion (Position 21).

SEQ ID NO:785: n indicates ta. (Position 21).

SEQ ID NO:797: n indicates acac. (Position 21).

SEQ ID NO:806: n indicates gatttgtggtatccag. (Position 21).

SEQ ID NO:808: n indicates ag or deletion (Position 21).

SEQ ID NO:809: n indicates ta or deletion (Position 21).

SEQ ID NO:815: n indicates t (Position 21).

SEQ ID NO:828: n indicates cagaggct (Position 21).

SEQ ID NO:830: n indicates ca or deletion (Position 21).

SEQ ID NO:831: n indicates ag or deletion (Position 21).

SEQ ID NO:843: n indicates gtaaa (Position 21).

SEQ ID NO:845: n indicates a (Position 21).

SEQ ID NO:888: n indicates tc (Position 21).

SEQ ID NO:890: n indicates t or deletion (Position 21).

SEQ ID NO:913: n indicates t or deletion (Position 21).

SEQ ID NO:932: n indicates t or deletion (Position 21).

SEQ ID NO:933: n indicates t or deletion (Position 21).

SEQ ID NO:955: n indicates at or deletion (Position 21).

SEQ ID NO:956: n indicates a or deletion (Position 21).

SEQ ID NO:957: n indicates c or deletion (Position 21).

SEQ ID NO:987: n indicates c (Position 21).

SEQ ID NO:999: n indicates gtt or deletion (Position 21).

SEQ ID NO:1164: n indicates at (Position 21).

SEQ ID NO:1166: n indicates c or deletion (Position 21).

SEQ ID NO:1167: n indicates t or deletion (Position 21).

SEQ ID NO:1168: n indicates t or deletion (Position 21).

SEQ ID NO:1169: n indicates g (Position 21).

SEQ ID NO:1171 n indicates c (Position 21).

SEQ ID NO:1173: n indicates t (Position 21).

SEQ ID NO:1175: n indicates c or deletion (Position 21).

SEQ ID NO:1200: n indicates a or deletion (Position 21).

SEQ ID NO:1204: n indicates a (Position 21).

SEQ ID NO:1207: n indicates tt (Position 21).

SEQ ID NO:1210: n indicates at (Position 21).

SEQ ID NO:1245: n indicates t (Position 21).

SEQ ID NO:1248: n indicates t or deletion (Position 21).

SEQ ID NO:1249: n indicates t (Position 21).

SEQ ID NO:1251: n indicates a or deletion (Position 21).

SEQ ID NO:1252: n indicates tgt or deletion (Position 21).

SEQ ID NO:1260: n indicates t or deletion (Position 21).

SEQ ID NO:1309: n indicates a or deletion (Position 21).

SEQ ID NO:1389: n indicates g or deletion (Position 21).

SEQ ID NO:1411: n indicates a or deletion (Position 21).

SEQ ID NO:1417: n indicates aaag (Position 21).

SEQ ID NO:1424: n indicates gtg or deletion (Position 21).

SEQ ID NO:1426: n indicates gg or tggtggggtgga (Position 21).

SEQ ID NO:1429: n indicates at or deletion (Position 21).

SEQ ID NO:1436: n indicates a (Position 21).

SEQ ID NO:1453: n indicates c or deletion (Position 21).

SEQ ID NO:1456: n indicates gg (Position 21).

SEQ ID NO:1465: n indicates gtc or deletion (Position 21).

SEQ ID NO:1487: n indicates t or deletion (Position 21).

SEQ ID NO:1494: n indicates tt (Position 21).

SEQ ID NO:1497: n indicates t repeated 9 to 12 times (Position 21).

SEQ ID NO:1499: n indicates a or deletion (Position 21).

SEQ ID NO:1501: n indicates a repeated 10 to 13 times (Position 21).

SEQ ID NO:1504: n indicates ct or deletion (Position 21).

SEQ ID NO:1507: n indicates cagatcttcttcagctaatttagaaatgt (Position 21).

SEQ ID NO:1533: n indicates a or deletion (Position 21).

SEQ ID NO:1540: n indicates c (Position 21).

SEQ ID NO:1545: n indicates t (Position 21).

SEQ ID NO:1552: n indicates t repeated 9 to 12 times (Position 21).

SEQ ID NO:1555: n indicates t (Position 21).

SEQ ID NO:1557: n indicates aaaaaaagaaaa (Position 21).

SEQ ID NO:1558: n indicates aaaaaaaaaaaa (Position 21).

SEQ ID NO:1559: n indicates aaaaaaaaaa (Position 21).

SEQ ID NO:1563: n indicates t or deletion (Position 21).

SEQ ID NO:1572: n indicates c (Position 21).

SEQ ID NO:1574: n indicates a or deletion (Position 21).

SEQ ID NO:1575: n indicates c or deletion (Position 21).

SEQ ID NO:1596: n indicates cct or deletion (Position 21).

SEQ ID NO:1598: n indicates tc (Position 21).

SEQ ID NO:1616: n indicates ca or deletion (Position 21).

SEQ ID NO:1638: n indicates g (Position 21).

SEQ ID NO:1661: n indicates t or deletion (Position 21).

SEQ ID NO:1690: n indicates gccag (Position 21).

SEQ ID NO:1718: n indicates t (Position 21).

SEQ ID NO:1723: n indicates c or deletion (Position 21).

SEQ ID NO:1729: n indicates tc or deletion (Position 21).

SEQ ID NO:1740: n indicates ct or deletion (Position 21).

SEQ ID NO:1771: n indicates a (Position 21).

SEQ ID NO:1781: n indicates a or deletion (Position 21).

SEQ ID NO:1787: n indicates t or deletion (Position 21).

SEQ ID NO:1791: n indicates t or deletion (Position 21).

SEQ ID NO:1792: n indicates g or deletion (Position 21).

SEQ ID NO:1800: n indicates t or deletion (Position 21).

SEQ ID NO:1801: n indicates t or deletion (Position 21).

SEQ ID NO:1802: n indicates a or deletion (Position 21).

SEQ ID NO:1815: n indicates a or deletion (Position 21).

SEQ ID NO:1819: n indicates ca or deletion (Position 21).

SEQ ID NO:1820: n indicates t or deletion (Position 21).

SEQ ID NO:1824: n indicates t or deletion (Position 21).

SEQ ID NO:1829: n indicates t or deletion (Position 21).

SEQ ID NO:1830: n indicates c or deletion (Position 21).

SEQ ID NO:1838: n indicates a or deletion (Position 21).

SEQ ID NO:1840: n indicates t or deletion (Position 21).

SEQ ID NO:1847: n indicates gatt or deletion (Position 21).

SEQ ID NO:1848: n indicates t (Position 21).

SEQ ID NO:1853: n indicates t or deletion (Position 21).

SEQ ID NO:1854: n indicates gt (Position 21).

SEQ ID NO:1857: n indicates a or deletion (Position 21).

SEQ ID NO:1858: n indicates a or deletion (Position 21).

SEQ ID NO:1862: n indicates t or deletion (Position 21).

SEQ ID NO:1865: n indicates at or deletion (Position 21).

SEQ ID NO:1871: n indicates a or deletion (Position 21).

SEQ ID NO:1874: n indicates t or deletion (Position 21).

SEQ ID NO:1877: n indicates at or deletion (Position 21).

SEQ ID NO:1878: n indicates a or deletion (Position 21).

SEQ ID NO:1879: n indicates t repeated 12 to 14 times (Position 21).

SEQ ID NO:1882: n indicates t or deletion (Position 21).

SEQ ID NO:1884: n indicates cac or deletion (Position 21).

SEQ ID NO:1891: n indicates cca (Position 21).

SEQ ID NO:1919: n indicates t or deletion (Position 21).

SEQ ID NO:1949: n indicates c or deletion (Position 21).

SEQ ID NO:1957: n indicates aaaa or deletion (Position 21).

SEQ ID NO:1970: n indicates c or deletion (Position 21).

SEQ ID NO:1980: n indicates t repeated 7 to 9 times (Position 21).

SEQ ID NO:1981: n indicates a or deletion (Position 21).

SEQ ID NO:1993: n indicates taac or deletion (Position 21).

SEQ ID NO:1994: n indicates ctcttt (Position 21).

SEQ ID NO:1995: n indicates ct (Position 21).

SEQ ID NO:2002: n indicates a or deletion (Position 21).

SEQ ID NO:2005: n indicates t or deletion (Position 21).

SEQ ID NO:2008: n indicates g or deletion (Position 21).

SEQ ID NO:2011: n indicates aattagaa or deletion (Position 21).

SEQ ID NO:2012: n indicates tttaaaa or ttttaa (Position 21).

SEQ ID NO:2015: n indicates t or deletion (Position 21).

SEQ ID NO:2020: n indicates t or deletion (Position 21).

SEQ ID NO:2024: n indicates g or deletion (Position 21).

SEQ ID NO:2025: n indicates t or deletion (Position 21).

SEQ ID NO:2030: n indicates aaa or deletion (Position 21).

SEQ ID NO:2031: n indicates a or deletion (Position 21).

SEQ ID NO:2042: n indicates c (Position 21).

SEQ ID NO:2072: n indicates a or deletion (Position 21).

SEQ ID NO:2074: n indicates a or deletion (Position 21).

SEQ ID NO:2243: n indicates tca repeated 14 to 16 times (Position 21).

SEQ ID NO:2244: n indicates a repeated 8 to 10 times (Position 21).

SEQ ID NO:2245: n indicates cacagtcat or deletion (Position 21).

SEQ ID NO:2246: n indicates tt or deletion (Position 21).

SEQ ID NO:2247: n indicates a repeated 10 to 12 times (Position 21).

SEQ ID NO:2248: n indicates c or deletion (Position 21).

SEQ ID NO:2249: n indicates a repeated 16 to 18 times (Position 21).

SEQ ID NO:2250: n indicates g (Position 21).

SEQ ID NO:2252: n indicates c or deletion (Position 21).

SEQ ID NO:2253: n indicates t or deletion (Position 21).

SEQ ID NO:2254: n indicates a or deletion (Position 21).

SEQ ID NO:2255: n indicates tg (Position 21).

SEQ ID NO:2257: n indicates t repeated 10 to 13 (Position 21).

SEQ ID NO:2258: n indicates gt repeated 11 to 13 times (Position 21).

SEQ ID NO:2259: n indicates a or deletion (Position 21).

SEQ ID NO:2260: n indicates g or deletion (Position 21).

SEQ ID NO:2261: n indicates g or deletion (Position 21).

SEQ ID NO:2262: n indicates t repeated 9 to 11 times (Position 21).

SEQ ID NO:2263: n indicates g (Position 21).

SEQ ID NO:2265: n indicates tt or deletion (Position 21).

SEQ ID NO:2266: n indicates a repeated 7 to 9 times (Position 21).

SEQ ID NO:2267: n indicates t repeated 9 to 11 times (Position 21).

SEQ ID NO:2268: n indicates a repeated 9 to 10 times (Position 21).

SEQ ID NO:2269: n indicates gt or deletion (Position 21).

SEQ ID NO:2270: n indicates a or deletion (Position 21).

SEQ ID NO:2271: n indicates t (Position 21).

SEQ ID NO:2273: n indicates a or deletion (Position 21).

SEQ ID NO:2274: n indicates ct or deletion (Position 21).

SEQ ID NO:2275: n indicates g or deletion (Position 21).

SEQ ID NO:2276: n indicates a or deletion (Position 21).

SEQ ID NO:2277: n indicates a or deletion (Position 21).

SEQ ID NO:2278: n indicates a or deletion (Position 21).

SEQ ID NO:2279: n indicates c or deletion (Position 21).

SEQ ID NO:2280: n indicates aaag or deletion (Position 21).

SEQ ID NO:2348: n indicates t repeated 22 to 26 times (Position 21).

SEQ ID NO:2349: n indicates g repeated 8 to 10 times (Position 21).

SEQ ID NO:2350: n indicates c repeated 6 to 7 times (Position 21).

SEQ ID NO:2351: n indicates a repeated 12 to 14 times (Position 21).

SEQ ID NO:2427: n indicates caccaggcagcagactctgatgaggaggggagggg (Position 21).

SEQ ID NO:2429: n indicates g (Position 21).

SEQ ID NO:2474: n indicates tcac or deletion (Position 21).

SEQ ID NO:2475: n indicates t or deletion (Position 21).

SEQ ID NO:2476: n indicates t repeated 9 to 11 times (Position 21).

SEQ ID NO:2477: n indicates a repeated 7 to 8 times (Position 21).

SEQ ID NO:2495: n indicates t repeated 13 to 16 times (Position 21).

SEQ ID NO:2496: n indicates t repeated 9 to 10 times (Position 21).

SEQ ID NO:2497: n indicates t repeated 14 to 16 times (Position 21).

SEQ ID NO:2498: n indicates t repeated 13 to 17 times (Position 21).

SEQ ID NO:2499: n indicates t (Position 21).

SEQ ID NO:2501: n indicates a repeated 8 to 9 times (Position 21).

SEQ ID NO:2502: n indicates t repeated 8 to 9 times (Position 21).

SEQ ID NO:2503: n indicates gcagtattactgtagt or deletion (Position 21).

SEQ ID NO:2504: n indicates t repeated 13 to 14 times (Position 21).

SEQ ID NO:2505: n indicates t repeated 9 to 10 times (Position 21).

SEQ ID NO:2506: n indicates t repeated 10 to 11 times (Position 21).

SEQ ID NO:2524: n indicates t or deletion (Position 21).

SEQ ID NO:2525: n indicates t repeated 12 to 15 times (Position 21).

SEQ ID NO:2586: n indicates a or deletion (Position 21).

SEQ ID NO:2587: n indicates at or deletion (Position 21).

SEQ ID NO:2594: n indicates t or deletion (Position 21).

SEQ ID NO:2595: n indicates ttc or deletion (Position 21).

SEQ ID NO:2606: n indicates ctt (Position 21).

SEQ ID NO:2651: n indicates c repeated 9 to 11 times (Position 21).

SEQ ID NO:2652: n indicates a repeated 15 to 21 times (Position 21).

SEQ ID NO:2653: n indicates ggggtggcggggtggg or deletion (Position 21).

SEQ ID NO:2654: n indicates t or deletion (Position 21).

SEQ ID NO:2655: n indicates a (Position 21).

SEQ ID NO:2657: n indicates a or deletion (Position 21).

SEQ ID NO:2658: n indicates t repeated 10 to 12 times (Position 21).

SEQ ID NO:2659: n indicates tt (Position 21).

SEQ ID NO:2661: n indicates tccctccttgaagctgatcgt or deletion (Position 21).

SEQ ID NO:2662: n indicates ca repeated 12 to 18 times (Position 21).

SEQ ID NO:2685: n indicates a repeated 18 to 20 times (Position 21).

SEQ ID NO:2686: n indicates aa (Position 21).

SEQ ID NO:2688: n indicates t or deletion (Position 21).

SEQ ID NO:2689: n indicates t repeated 9 to 13 times (Position 21).

SEQ ID NO:2690: n indicates aa or deletion (Position 21).

SEQ ID NO:2691: n indicates ttgaca or gtccaatat (Position 21).

SEQ ID NO:2692: n indicates cta or deletion (Position 21).

SEQ ID NO:2693: n indicates t repeated 9 to 10 times (Position 21).

SEQ ID NO:2694: n indicates gagatgttgtggctcacat (Position 21).

SEQ ID NO:2696: n indicates cc or deletion (Position 21).

SEQ ID NO:2697: n indicates act or deletion (Position 21).

SEQ ID NO:2755: n indicates tat or deletion (Position 21).

SEQ ID NO:2756: n indicates ac repeated 14 to 17 times (Position 21).

SEQ ID NO:2757: n indicates a repeated 16 to 27 times (Position 21).

SEQ ID NO:2758: n indicates t or deletion (Position 21).

SEQ ID NO:2759: n indicates a repeated 8 to 10 times (Position 21).

SEQ ID NO:2760: n indicates gt repeated 9 to 11 times (Position 21).

SEQ ID NO:2761: n indicates aa or deletion (Position 21).

SEQ ID NO:2762: n indicates t or deletion (Position 21).

SEQ ID NO:2763: n indicates ac repeated 8 to 12 times (Position 21).

SEQ ID NO:2764: n indicates a or deletion (Position 21).

SEQ ID NO:2810: n indicates a (Position 21).

SEQ ID NO:2812: n indicates aa or deletion (Position 21).

SEQ ID NO:2813: n indicates ca or deletion (Position 21).

SEQ ID NO:2814: n indicates t or deletion (Position 21).

SEQ ID NO:2815: n indicates tgtgtg or deletion (Position 21).

SEQ ID NO:2912: n indicates a (Position 21).

SEQ ID NO:2914: n indicates g (Position 21).

SEQ ID NO:2916: n indicates actt or deletion (Position 21).

SEQ ID NO:2917: n indicates ttta or deletion (Position 21).

SEQ ID NO:2918: n indicates a repeated 11 to 13 times (Position 21).

SEQ ID NO:2919: n indicates t repeated 8 to 10 times (Position 21).

SEQ ID NO:2920: n indicates a repeated 12 to 14 times (Position 21).

SEQ ID NO:2921: n indicates cttgta or deletion (Position 21).

SEQ ID NO:2922: n indicates a repeated 9 to 10 times (Position 21).

SEQ ID NO:2923: n indicates c or deletion (Position 21).

SEQ ID NO:2924: n indicates ctt (Position 21).

SEQ ID NO:2926: n indicates a or deletion (Position 21).

SEQ ID NO:2927: n indicates a repeated 9 to 10 times (Position 21).

SEQ ID NO:2928: n indicates tgt or deletion (Position 21).

SEQ ID NO:2929: n indicates a repeated 24 to 27 times (Position 21).

SEQ ID NO:2930: n indicates ta repeated 10 to 21 times (Position 21).

SEQ ID NO:2931: n indicates a repeated 8 to 10 times (Position 21).

SEQ ID NO:2932: n indicates a repeated 11 to 13 times (Position 21).

SEQ ID NO:2933: n indicates a repeated 8 to 10 times (Position 21).

SEQ ID NO:2999: n indicates tatc or deletion (Position 21).

SEQ ID NO:3000: n indicates atattcacttggtatctg or deletion (Position 21).

SEQ ID NO:3001: n indicates tt ta or deletion (Position 21).

SEQ ID NO:3002: n indicates t (Position 21).

SEQ ID NO:3004: n indicates g or deletion (Position 21).

SEQ ID NO:3005: n indicates a or deletion (Position 21).

SEQ ID NO:3006: n indicates a repeated 9 to 11 times (Position 21).

SEQ ID NO:3007: n indicates g or deletion (Position 21).

SEQ ID NO:3008: n indicates at repeated 4 to 5 times (Position 21).

SEQ ID NO:3009: n indicates t repeated 7 to 8 times (Position 21).

SEQ ID NO:3010: n indicates t repeated 19 to 23 times (Position 21).

SEQ ID NO:3011: n indicates t or deletion (Position 21).

SEQ ID NO:3012: n indicates tgat or deletion (Position 21).

SEQ ID NO:3013: n indicates t repeated 8 to 10 times (Position 21).

SEQ ID NO:3014: n indicates a or deletion (Position 21).

SEQ ID NO:3021: n indicates a repeated 13 to 15 times (Position 21).

SEQ ID NO:3022: n indicates t repeated 12 to 15 times (Position 21).

SEQ ID NO:3042: n indicates g (Position 21).

SEQ ID NO:3044: n indicates a or deletion (Position 21).

SEQ ID NO:3046: n indicates g or deletion (Position 21).

SEQ ID NO:3047: n indicates t repeated 11 to 13 times (Position 21).

SEQ ID NO:3049: n indicates a or deletion (Position 21).

SEQ ID NO:3051: n indicates t repeated 9 to 11 times (Position 21).

SEQ ID NO:3054: n indicates t or deletion (Position 21).

SEQ ID NO:3056: n indicates t or deletion (Position 21).

SEQ ID NO:3060: n indicates t or deletion (Position 21).

SEQ ID NO:3065: n indicates aaga (Position 21).

SEQ ID NO:3069: n indicates aaaa or deletion (Position 21).

SEQ ID NO:3073: n indicates t repeated 9 to 13 times (Position 21).

SEQ ID NO:3081: n indicates a or deletion (Position 21).

SEQ ID NO:3103: n indicates t repeated 11 to 13 times (Position 21).

SEQ ID NO:3119: n indicates acta (Position 21).

SEQ ID NO:3125: n indicates gtg or deletion (Position 21).

SEQ ID NO:3130: n indicates t repeated 11 to 12 times (Position 21).

SEQ ID NO:3140: n indicates tta or deletion (Position 21).

SEQ ID NO:3154: n indicates g (Position 21).

SEQ ID NO:3156: n indicates a (Position 21).

SEQ ID NO:3158: n indicates cct or deletion (Position 21).

SEQ ID NO:3169: n indicates gga or deletion (Position 21).

SEQ ID NO:3179: n indicates t repeated 12 to 14 times (Position 21).

SEQ ID NO:3184: n indicates t repeated 16 to 17 times (Position 21).

SEQ ID NO:3196: n indicates g (Position 21).

SEQ ID NO:3273: n indicates ag (Position 21).

SEQ ID NO:3306: n indicates g (Position 21).

SEQ ID NO:3310: n indicates c (Position 21).

SEQ ID NO:3315: n indicates ct or deletion (Position 21).

SEQ ID NO:3317: n indicates gc or deletion (Position 21).

SEQ ID NO:3352: n indicates t repeated 9 to 11 times (Position 21).

SEQ ID NO:3355: n indicates a (Position 21).

SEQ ID NO:3358: n indicates t or deletion (Position 21).

SEQ ID NO:3510 [symbol] n represents at or deletion (Location 21).

SEQ ID NO:3512 [symbol] n represents c or deletion (Location 21).

SEQ ID NO:3513 [symbol] n represents t or deletion (Location 21).

SEQ ID NO:3514 [symbol] n represents t or deletion (Location 21).

SEQ ID NO:3515 [symbol] n represents g or deletion (Location 21).

SEQ ID NO:3517 [symbol] n represents c or deletion (Location 21).

SEQ ID NO:3519 [symbol] n represents t or deletion (Location 21).

SEQ ID NO:3521 [symbol] n represents c or deletion (Location 21).

SEQ ID NO:3649 [symbol] n represents 14 to 16 repeats of tca (from Location 21).

SEQ ID NO:3650 [symbol] n represents 8 to 10 repeats of a (from Location 21).

SEQ ID NO:3651 [symbol] n represents cacagtcat or deletion (Location 21).

SEQ ID NO3652 [symbol] n represents tt or deletion (Location 21).

SEQ ID NO:3653 [symbol] n represents 10 to 12 repeats of a (from Location 21).

SEQ ID NO:3654 [symbol] n represents c or deletion (Location 21).

SEQ ID NO:3655 [symbol] n represents 16 to 18 repeats of a (from Location 21).

SEQ ID NO:3656 [symbol] n represents g or deletion (Location 21).

SEQ ID NO:3658 [symbol] n represents c or deletion (Location 21).

SEQ ID NO:3659 [symbol] n represents t or deletion (Location 21).

SEQ ID NO:3660 [symbol] n represents a or deletion (Location 21).

SEQ ID NO:3661 [symbol] n represents tg or deletion (Location 21).

SEQ ID NO:3663 [symbol] n represents 10 to 13 repeats of t (from Location 21).

SEQ ID NO:3664 [symbol] n represents 11 to 13 repeats of gt (from Location 21).

SEQ ID NO:3665 [symbol] n represents a or deletion (Location 21).

SEQ ID NO:3666 [symbol] n represents g or deletion (Location 21).

SEQ ID NO:3667 [symbol] n represents g or deletion (Location 21).

SEQ ID NO:3668 [symbol] n represents 9 to 11 repeats of t (from Location 21).

SEQ ID NO:3669 [symbol] n represents g or deletion (Location 21).

SEQ ID NO:3671 [symbol] n represents tt or deletion (Location 21).

SEQ ID NO:3672 [symbol] n represents 7 to 9 repeats of a (from Location 21).

SEQ ID NO:3673 [symbol] n represents 9 to 11 repeats of t (from Location 21).

SEQ ID NO:3674 [symbol] n represents 9 to 10 repeats of a (from Location 21).

SEQ ID NO:3675 [symbol] n represents gt or deletion (Location 21).

SEQ ID NO:3676 [symbol] n represents a or deletion (Location 21).

SEQ ID NO:3677 [symbol] n represents t or deletion (Location 21).

SEQ ID NO:3679 [symbol] n represents a or deletion (Location 21).

SEQ ID NO:3680 [symbol] n represents ct or deletion (Location 21).

SEQ ID NO:3681 [symbol] n represents g or deletion (Location 21).

SEQ ID NO:3682 [symbol] n represents a or deletion (Location 21).

SEQ ID NO:3683 [symbol] n represents a or deletion (Location 21).

SEQ ID NO:3684 [symbol] n represents a or deletion (Location 21).

SEQ ID NO:3685 [symbol] n represents c or deletion (Location 21).

SEQ ID NO:3686 [symbol] n represents aaag or deletion (Location 21).

SEQ ID NO:3751 [symbol] n represents 22 to 26 repeats of t (from Location 21).

SEQ ID NO:3752 [symbol] n represents 8 to 10 repeats of g (from Location 21).

SEQ ID NO:3753 [symbol] n represents 6 to 7 repeats of c (from Location 21).

SEQ ID NO:3754 [symbol] n represents 12 to 14 repeats of a (from Location 21).

SEQ ID NO:3833 [symbol] n represents tt or deletion (Location 21).

SEQ ID NO:3834 [symbol] n represents 9 to 11 repeats of a (from Location 21).

SEQ ID NO:3835 [symbol] n represents 8 to 12 repeats of a (from Location 21).

SEQ ID NO:3836 [symbol] n represents t or deletion (Location 21).

SEQ ID NO:3837 [symbol] n represents t or deletion (Location 21).

SEQ ID NO:3838 [symbol] n represents t or deletion (Location 21).

SEQ ID NO:3839 [symbol] n represents a or deletion (Location 21).

SEQ ID NO:3840 [symbol] n represents t or deletion (Location 21).

SEQ ID NO:3841 [symbol] n represents t or deletion (Location 21).

SEQ ID NO:3842 [symbol] n represents 11 to 15 repeats of t (from Location 21).

SEQ ID NO:3843 [symbol] n represents cat or deletion (Location 21).

SEQ ID NO:3844 [symbol] n represents t or deletion (Location 21).

SEQ ID NO:3845 [symbol] n represents a or deletion (Location 21).

SEQ ID NO:3846 [symbol] n represents a or deletion (Location 21).

SEQ ID NO:3847 [symbol] n represents t or deletion (Location 21).

SEQ ID NO:3848 [symbol] n represents a or deletion (Location 21).

SEQ ID NO:3857 [symbol] n represents g or deletion (Location 21).

SEQ ID NO:3879 [symbol] n represents a or deletion (Location 21).

SEQ ID NO:3885 [symbol] n represents aaag or deletion (Location 21).

SEQ ID NO:3915 [symbol] n represents t or deletion (Location 21).

SEQ ID NO:3918 [symbol] n represents a or deletion (Location 21).

SEQ ID NO:3926 [symbol] n represents at or deletion (Location 21).

SEQ ID NO:3933 [symbol] n represents a or deletion (Location 21).

SEQ ID NO:3950 [symbol] n represents c or deletion (Location 21).

SEQ ID NO:3953 [symbol] n represents gg or deletion (Location 21).

SEQ ID NO:3962 [symbol] n represents gtc or deletion (Location 21).

SEQ ID NO:3984 [symbol] n represents t or deletion (Location 21).

SEQ ID NO:3991 [symbol] n represents tt or deletion (Location 21).

SEQ ID NO:3994 [symbol] n represents 9 to 12 repeats of t (from Location 21).

SEQ ID NO:3996 [symbol] n represents a or deletion (Location 21).

SEQ ID NO:3998 [symbol] n represents 10 to 13 repeats of a (from Location 21).

SEQ ID NO:4001 [symbol] n represents ct or deletion (Location 21).

SEQ ID NO:4004 [symbol] n represents cagatcttcttcagctaatttagaaatgt or deletion (Location 21).

SEQ ID NO:4030 [symbol] n represents a or deletion (Location 21).

SEQ ID NO:4037 [symbol] n represents c or deletion (Location 21).

SEQ ID NO:4042 [symbol] n represents t or deletion (Location 21).

SEQ ID NO:4049 [symbol] n represents 9 to 12 repeats of t (from Location 21).

SEQ ID NO:4052 [symbol] n represents t or deletion (Location 21).

SEQ ID NO:4054 [symbol] n represents g (a)4, a (a)4 or a (Location 21).

SEQ ID NO:4058 [symbol] n represents t or deletion (Location 21).

SEQ ID NO:4067 [symbol] n represents c or deletion (Location 21).

SEQ ID NO:4069 [symbol] n represents a or deletion (Location 21).

SEQ ID NO:4070 [symbol] n represents c or deletion (Location 21).

SEQ ID NO:4077 [symbol] n represents g or deletion (Location 21).

SEQ ID NO:4079 [symbol] n represents 18 to 20 repeats of t (from Location 21).

SEQ ID NO:4084 [symbol] n represents 11 to 13 repeats of a (from Location 21).

SEQ ID NO:4085 [symbol] n represents gaaa or deletion (Location 21).

SEQ ID NO:4089 [symbol] n represents 10 to 12 repeats of a (from Location 21).

SEQ ID NO:4092 [symbol] n represents c or deletion (Location 21).

SEQ ID NO:4102 [symbol] n represents ca or deletion (Location 21).

SEQ ID NO:4109 [symbol] n represents at or deletion (Location 21).

SEQ ID NO:4113 [symbol] n represents ctt or deletion (Location 21).

SEQ ID NO:4115 [symbol] n represents g or deletion (Location 21).

SEQ ID NO:4117 [symbol] n represents ggggct or deletion (Location 21).

SEQ ID NO:4121 [symbol] n represents 19 to 22 repeats of t (from Location 21).

SEQ ID NO:4126 [symbol] n represents 6 to 7 repeats of t (from Location 21).

SEQ ID NO:4129 [symbol] n represents 11 to 13 repeats of t (from Location 21).

SEQ ID NO:4173 [symbol] n represents 7 to 8 repeats of c (from Location 21).

SEQ ID NO:4175 [symbol] n represents 10 to 12 repeats of a (from Location 21).

SEQ ID NO:4183 [symbol] n represents c or deletion (Location 21).

SEQ ID NO:4188 [symbol] n represents aaga or deletion (Location 21).

SEQ ID NO:4190 [symbol] n represents 9 to 11 repeats of a (from Location 21).

SEQ ID NO:4193 [symbol] n represents ct or deletion (Location 21).

SEQ ID NO:4198 [symbol] n represents 8 to 9 repeats of t (from Location 21).

SEQ ID NO:4218 [symbol] n represents g or deletion (Location 21).

SEQ ID NO:4224 [symbol] n represents cttt or deletion (Location 21).

SEQ ID NO:4229 [symbol] n represents t or deletion (Location 21).

SEQ ID NO:4234 [symbol] n represents c or deletion (Location 21).

SEQ ID NO:4235 [symbol] n represents a or deletion (Location 21).

SEQ ID NO:4238 [symbol] n represents gtt or deletion (Location 21).

SEQ ID NO:4239 [symbol] n represents t or deletion (Location 21).

SEQ ID NO:4259 [symbol] n represents at or deletion (Location 21).

SEQ ID NO:4273 [symbol] n represents g or deletion (Location 21).

SEQ ID NO:4280 [symbol] n represents 15 to 17 repeats of a (from Location 21).

SEQ ID NO:4294 [symbol] n represents t or deletion (Location 21).

SEQ ID NO:4298 [symbol] n represents t or deletion (Location 21).

SEQ ID NO:4310 [symbol] n represents t or deletion (Location 21).

SEQ ID NO:4314 [symbol] n represents a or deletion (Location 21).

SEQ ID NO:4315 [symbol] n represents 13 to 15 repeats of t (from Location 21).

SEQ ID NO:4316 [symbol] n represents 12 to 13 repeats of a (from Location 21).

SEQ ID NO:4317 [symbol] n represents t or deletion (Location 21).

SEQ ID NO:4319 [symbol] n represents t or deletion (Location 21).

SEQ ID NO:4320 [symbol] n represents 13 to 15 repeats of a (from Location 21).

SEQ ID NO:4325 [symbol] n represents a or deletion (Location 21).

SEQ ID NO:4331 [symbol] n represents 5 to 11 repeats of t (from Location 21).

SEQ ID NO:4333 [symbol] n represents 8 to 9 repeats of t (from Location 21).

SEQ ID NO:4334 [symbol] n represents t or deletion (Location 21).

SEQ ID NO:4345 [symbol] n represents 9 to 10 repeats of t (from Location 21).

SEQ ID NO:4348 [symbol] n represents 10 to 11 repeats of a (from Location 21).

SEQ ID NO:4354 [symbol] n represents a or deletion (Location 21).

SEQ ID NO:4361 [symbol] n represents a or deletion (Location 21).

SEQ ID NO:4372 [symbol] n represents ct or deletion (Location 21).

SEQ ID NO:4391 [symbol] n represents t or deletion (Location 21).

SEQ ID NO:4397 [symbol] n represents a or deletion (Location 21).

SEQ ID NO:4398 [symbol] n represents at or deletion (Location 21).

SEQ ID NO:4408 [symbol] n represents tgtccaaaggaaggacacg or deletion (Location 21).

SEQ ID NO:4414 [symbol] n represents 6 to 8 repeats of tc (from Location 21).

SEQ ID NO:4416 [symbol] n represents c or deletion (Location 21).

SEQ ID NO:4419 [symbol] n represents t or deletion (Location 21).

SEQ ID NO:4424 [symbol] n represents t or deletion (Location 21).

SEQ ID NO:4425 [symbol] n represents c or deletion (Location 21).

SEQ ID NO:4433 [symbol] n represents a or deletion (Location 21).

SEQ ID NO:4435 [symbol] n represents t or deletion (Location 21).

SEQ ID NO:4442 [symbol] n represents 6 to 7 repeats of gatt (from Location 21).

SEQ ID NO:4443 [symbol] n represents t or deletion (Location 21).

SEQ ID NO:4448 [symbol] n represents t or deletion (Location 21).

SEQ ID NO:4449 [symbol] n represents gt or deletion (Location 21).

SEQ ID NO:4452 [symbol] n represents a or deletion (Location 21).

SEQ ID NO:4453 [symbol] n represents a or deletion (Location 21).

SEQ ID NO:4457 [symbol] n represents t or deletion (Location 21).

SEQ ID NO:4460 [symbol] n represents at or deletion (Location 21).

SEQ ID NO:4466 [symbol] n represents a or deletion (Location 21).

SEQ ID NO:4469 [symbol] n represents t or deletion (Location 21).

SEQ ID NO:4472 [symbol] n represents at or deletion (Location 21).

SEQ ID NO:4473 [symbol] n represents a or deletion (Location 21).

SEQ ID NO:4474 [symbol] n represents 12 to 14 repeats of t (from Location 21).

SEQ ID NO:4477 [symbol] n represents t or deletion (Location 21).

SEQ ID NO:4479 [symbol] n represents cac or deletion (Location 21).

SEQ ID NO:4486 [symbol] n represents cca or deletion (Location 21).

SEQ ID NO:4514 [symbol] n represents t or deletion (Location 21).

SEQ ID NO:4544 [symbol] n represents c or deletion (Location 21).

SEQ ID NO:4552 [symbol] n represents aaaa or deletion (Location 21).

SEQ ID NO:4565 [symbol] n represents c or deletion (Location 21).

SEQ ID NO:4575 [symbol] n represents 8 to 9 repeats of t (from Location 21).

SEQ ID NO:4576 [symbol] n represents a or deletion (Location 21).

SEQ ID NO:4588 [symbol] n represents taac or deletion (Location 21).

SEQ ID NO:4589 [symbol] n represents ctcttt or deletion (Location 21).

SEQ ID NO:4590 [symbol] n represents ct or deletion (Location 21).

SEQ ID NO:4597 [symbol] n represents a or deletion (Location 21).

SEQ ID NO:4600 [symbol] n represents t or deletion (Location 21).

SEQ ID NO:4603 [symbol] n represents g or deletion (Location 21).

SEQ ID NO:4606 [symbol] n represents aattagaa or deletion (Location 21).

SEQ ID NO:4607 [symbol] n represents tttaaaa or ttttaa (Location 21).

SEQ ID NO:4610 [symbol] n represents t or deletion (Location 21).

SEQ ID NO:4615 [symbol] n represents t or deletion (Location 21).

SEQ ID NO:4627 [symbol] n represents c or deletion (Location 21).

SEQ ID NO:4652 [symbol] n represents 11 to 14 repeats of t (from Location 21).

SEQ ID NO:4653 [symbol] n represents t or deletion (Location 21).

SEQ ID NO:4654 [symbol] n represents 10 to 13 repeats of t (from Location 21).

SEQ ID NO:4655 [symbol] n represents t or deletion (Location 21).

SEQ ID NO:4657 [symbol] n represents t or deletion (Location 21).

SEQ ID NO:4658 [symbol] n represents ta or deletion (Location 21).

SEQ ID NO:4660 [symbol] n represents 13 to 15 repeats of t (from Location 21).

SEQ ID NO:4661 [symbol] n represents c or deletion (Location 21).

SEQ ID NO:4662 [symbol] n represents 17 to 20 repeats of a (from Location 21).

SEQ ID NO:4663 [symbol] n represents 13 to 13 repeats of t (from Location 21).

SEQ ID NO:4664 [symbol] n represents 8 to 9 repeats of t (from Location 21).

SEQ ID NO:4665 [symbol] n represents 10 to 11 repeats of a (from Location 21).

SEQ ID NO:4666 [symbol] n represents 16 to 19 repeats of a (from Location 21).

SEQ ID NO:4758 [symbol] n represents g or deletion (Location 21).

SEQ ID NO:4760 [symbol] n represents 6 to 7 repeats of a (from Location 21).

SEQ ID NO:4761 [symbol] n represents c or deletion (Location 21).

SEQ ID NO:4763 [symbol] n represents tcctcaggg or deletion (Location 21).

SEQ ID NO:4764 [symbol] n represents 8 to 10 repeats of cgc (from Location 21).

SEQ ID NO:4765 [symbol] n represents 10 to 12 repeats of a (from Location 21).

SEQ ID NO:4766 [symbol] n represents caccaggcagcagactctgatgaggaggggaggggg or deletion (Location 21).

SEQ ID NO:4768 [symbol] n represents g or deletion (Location 21).

SEQ ID NO:4808 [symbol] n represents tcac or deletion (Location 21).

SEQ ID NO:4809 [symbol] n represents t or deletion (Location 21).

SEQ ID NO:4810 [symbol] n represents 9 to 11 repeats of t (from Location 21).

SEQ ID NO:4811 [symbol] n represents 7 to 8 repeats of a (from Location 21).

SEQ ID NO:4847 [symbol] n represents agg or deletion (Location 21).

SEQ ID NO:4848 [symbol] n represents taacatt or deletion (Location 21).

SEQ ID NO:4849 [symbol] n represents 10 to 12 repeats of a (from Location 21).

SEQ ID NO:4850 [symbol] n represents 15 to 17 repeats of t (from Location 21).

SEQ ID NO:4851 [symbol] n represents 11 to 13 repeats of a (from Location 21).

SEQ ID NO:4877 [symbol] n represents 11 to 13 repeats of t (from Location 21).

SEQ ID NO:4878 [symbol] n represents t or deletion (Location 21).

SEQ ID NO:4879 [symbol] n represents t or deletion (Location 21).

SEQ ID NO:4880 [symbol] n represents 10 to 12 repeats of a (from Location 21).

SEQ ID NO:4881 [symbol] n represents t or deletion (Location 21)

SEQ ID NO:4883 [symbol] n represents 7 to 9 repeats of c (from Location 21).

SEQ ID NO:4884 [symbol] n represents a or deletion (Location 21)

SEQ ID NO:4891 [symbol] n represents 13 to 16 repeats of t (from Location 21).

SEQ ID NO:4892 [symbol] n represents 9 to 10 repeats of t (from Location 21).

SEQ ID NO:4893 [symbol] n represents 14 to 16 repeats of t (from Location 21).

SEQ ID NO:4894 [symbol] n represents 13 to 17 repeats of t (from Location 21).

SEQ ID NO:4895 [symbol] n represents t or deletion (Location 21).

SEQ ID NO:4897 [symbol] n represents 8 to 9 repeats of a (from Location 21).

SEQ ID NO:4898 [symbol] n represents 8 to 9 repeats of t (from Location 21).

SEQ ID NO:4899 [symbol] n represents gcagtattactgtagt or deletion (Location 21).

SEQ ID NO:4900 [symbol] n represents 13 to 14 repeats of t (from Location 21).

SEQ ID NO:4901 [symbol] n represents 9 to 10 repeats of t (from Location 21).

SEQ ID NO:4902 [symbol] n represents 10 to 11 repeats of t (from Location 21).

SEQ ID NO:4907 [symbol] n represents 10 to 14 repeats of a (from Location 21).

SEQ ID NO:4908 [symbol] n represents 13 to 15 repeats of a (from Location 21).

SEQ ID NO:4909 [symbol] n represents a or deletion (Location 21).

SEQ ID NO:4910 [symbol] n represents t or deletion (Location 21).

SEQ ID NO:4918 [symbol] n represents 13 to 15 repeats of a (from Location 21).

SEQ ID NO:4919 [symbol] n represents 12 to 15 repeats of a (from Location 21).

SEQ ID NO:4936 [symbol] n represents g or deletion (Location 21).

SEQ ID NO:4938 [symbol] n represents aa or deletion (Location 21).

SEQ ID NO:4983 [symbol] n represents a or deletion (Location 21).

SEQ ID NO:4985 [symbol] n represents aa or deletion (Location 21).

SEQ ID NO:4986 [symbol] n represents ca or deletion (Location 21).

SEQ ID NO:4987 [symbol] n represents t or deletion (Location 21).

SEQ ID NO:4988 [symbol] n represents tgtgtg or deletion (Location 21).

SEQ ID NO:5076 [symbol] n represents a or deletion (Location 21).

SEQ ID NO:5078 [symbol] n represents g or deletion (Location 21).

SEQ ID NO:5080 [symbol] n represents actt or deletion (Location 21).

SEQ ID NO:5081 [symbol] n represents ttta or deletion (Location 21).

SEQ ID NO:5082 [symbol] n represents 11 to 13 repeats of a (from Location 21).

SEQ ID NO:5083 [symbol] n represents 8 to 10 repeats of t (from Location 21).

SEQ ID NO:5084 [symbol] n represents 12 to 14 repeats of a (from Location 21).

SEQ ID NO:5085 [symbol] n represents cttgta or deletion (Location 21).

SEQ ID NO:5086 [symbol] n represents 9 to 10 repeats of a (from Location 21).

SEQ ID NO:5087 [symbol] n represents ctt or deletion (Location 21).

SEQ ID NO:5088 [symbol] n represents ctt or deletion (Location 21).

SEQ ID NO:5090 [symbol] n represents a or deletion (Location 21).

SEQ ID NO:5091 [symbol] n represents 9 to 11 repeats of a (from Location 21)

SEQ ID NO:5092 [symbol] n represents tgt or deletion (Location 21).

SEQ ID NO:5093 [symbol] n represents 24 to 27 repeats of a (from Location 21)

SEQ ID NO:5094 [symbol] n represents 10 to 21 repeats of ta (from Location 21)

SEQ ID NO:5095 [symbol] n represents 8 to 10 repeats of a (from Location 21)

SEQ ID NO:5096 [symbol] n represents 11 to 13 repeats of a (from Location 21)

SEQ ID NO:5097 [symbol] n represents 8 to 10 repeats of a (from Location 21)

SEQ ID NO:5155 [symbol] n represents ctat or deletion (Location 21).

SEQ ID NO:5156 [symbol] n represents atattcacttggtatctg or deletion (Location 21).

SEQ ID NO:5157 [symbol] n represents ttta or deletion (Location 21).

SEQ ID NO:5158 [symbol] n represents t or deletion (Location 21).

SEQ ID NO:5160 [symbol] n represents g or deletion (Location 21).

SEQ ID NO:5161 [symbol] n represents a or deletion (Location 21).

SEQ ID NO:5162 [symbol] n represents 9 to 11 repeats of a (from Location 21).

SEQ ID NO:5163 [symbol] n represents g or deletion (Location 21).

SEQ ID NO:5164 [symbol] n represents 4 to 5 repeats of at (from Location 21).

SEQ ID NO:5165 [symbol] n represents 7 to 8 repeats of t (from Location 21).

SEQ ID NO:5166 [symbol] n represents 19 to 23 repeats of t (from Location 21).

SEQ ID NO:5167 [symbol] n represents t or deletion (Location 21).

SEQ ID NO:5168 [symbol] n represents tgat or deletion (Location 21).

SEQ ID NO:5169 [symbol] n represents 8 to 10 repeats of t (from Location 21).

SEQ ID NO:5170 [symbol] n represents a or deletion (Location 21).

SEQ ID NO:5187 [symbol] n represents gtg or deletion (Location 21).

SEQ ID NO:5189 [symbol] n represents gg or tggtggggtgga (Location 21).

SEQ ID NO:5209 [symbol] n represents acaaca or deletion (Location 21).

SEQ ID NO:5210 [symbol] n represents 11 to 13 repeats of t (from Location 21).

SEQ ID NO:5212 [symbol] n represents 15 to 18 repeats of ac (from Location 21).

SEQ ID NO:5218 [symbol] n represents 18 to 26 repeats of t (from Location 21).

SEQ ID NO:5227 [symbol] n represents tc or deletion (Location 21).

SEQ ID NO:5231 [symbol] n represents 16 to 18 repeats of t (from Location 21).

SEQ ID NO:5246 [symbol] n represents 18 to 20 repeats of t (from Location 21).

SEQ ID NO:5247 [symbol] n represents tggtaagt or deletion (Location 21).

SEQ ID NO:5249 [symbol] n represents t or deletion (Location 21).

SEQ ID NO:5255 [symbol] n represents g or deletion (Location 21).

SEQ ID NO:5256 [symbol] n represents g or deletion (Location 21).

SEQ ID NO:5257 [symbol] n represents c or deletion (Location 21).

SEQ ID NO:5258 [symbol] n represents ctct or deletion (Location 21).

SEQ ID NO:5261 [symbol] n represents a or deletion (Location 21).

SEQ ID NO:5264 [symbol] n represents t or deletion (Location 21).

SEQ ID NO:5271 [symbol] n represents 14 to 17 repeats of t (from Location 21).

SEQ ID NO:5276 [symbol] n represents 12 to 15 repeats of t (from Location 21).

SEQ ID NO:5277 [symbol] n represents 10 to 13 repeats of a (from Location 21).

SEQ ID NO:5278 [symbol] n represents 25 to 27 repeats of a (from Location 21).

SEQ ID NO:5299 [symbol] n represents c or deletion (Location 21).

SEQ ID NO:5308 [symbol] n represents 20 to 24 repeats of t (from Location 21).

SEQ ID NO:5311 [symbol] n represents t or deletion (Location 21).

SEQ ID NO:5312 [symbol] n represents t or deletion (Location 21).

SEQ ID NO:5314 [symbol] n represents g or deletion (Location 21).

SEQ ID NO:5320 [symbol] n represents 18 to 23 repeats of t (from Location 21).

SEQ ID NO:5340 [symbol] n represents c or deletion (Location 21).

SEQ ID NO:5400 [symbol] n represents a or deletion (Location 21).

SEQ ID NO:5404 [symbol] n represents a or deletion (Location 21).

SEQ ID NO:5407 [symbol] n represents tt or deletion (Location 21).

SEQ ID NO:5410 [symbol] n represents at or deletion (Location 21).

SEQ ID NO:5436 [symbol] n represents tgt or deletion (Location 21).

SEQ ID NO:5445 [symbol] n represents t or deletion (Location 21).

SEQ ID NO:5550 [symbol] n represents t or deletion (Location 21).

SEQ ID NO:5556 [symbol] n represents g or deletion (Location 21).

SEQ ID NO:5557 [symbol] n represents 11 to 13 repeats of t (from Location 21).

SEQ ID NO:5559 [symbol] n represents a or deletion (Location 21).

SEQ ID NO:5561 [symbol] n represents 9 to 11 repeats of t (from Location 21).

SEQ ID NO:5564 [symbol] n represents t or deletion (Location 21).

SEQ ID NO:5566 [symbol] n represents t or deletion (Location 21).

SEQ ID NO:5570 [symbol] n represents t or deletion (Location 21).

SEQ ID NO:5575 [symbol] n represents aaga or deletion (Location 21).

SEQ ID NO:5579 [symbol] n represents aaaa or deletion (Location 21).

SEQ ID NO:5583 [symbol] n represents 9 to 11 repeats of t (from Location 21).

SEQ ID NO:5591 [symbol] n represents a or deletion (Location 21).

SEQ ID NO:5614 [symbol] n represents 11 to 13 repeats of t (from Location 21).

SEQ ID NO:5630 [symbol] n represents acta or deletion (Location 21).

SEQ ID NO:5636 [symbol] n represents gtg or deletion (Location 21).

SEQ ID NO:5641 [symbol] n represents 11 to 12 repeats of t (from Location 21).

SEQ ID NO:5651 [symbol] n represents tta or deletion (Location 21).

SEQ ID NO:5665 [symbol] n represents g or deletion (Location 21).

SEQ ID NO:5667 [symbol] n represents a or deletion (Location 21).

SEQ ID NO:5669 [symbol] n represents cct or deletion (Location 21).

SEQ ID NO:5680 [symbol] n represents gga or deletion (Location 21).

SEQ ID NO:5690 [symbol] n represents 12 to 14 repeats of t (from Location 21).

SEQ ID NO:5695 [symbol] n represents 16 to 17 repeats of t (from Location 21).

SEQ ID NO:5707 [symbol] n represents g or deletion (Location 21).

SEQ ID NO:5740 [symbol] n represents c or deletion (Location 21).

SEQ ID NO:5800 [symbol] n represents ag or deletion (Location 21).

SEQ ID NO:5806 [symbol] n represents g or deletion (Location 21).

SEQ ID NO:5807 [symbol] n represents a or deletion (Location 21).

SEQ ID NO:5835 [symbol] n represents g or deletion (Location 21).

SEQ ID NO:5839 [symbol] n represents c or deletion (Location 21).

SEQ ID NO:5844 [symbol] n represents ct or deletion (Location 21).

SEQ ID NO:5846 [symbol] n represents gc or deletion (Location 21).

SEQ ID NO:5849 [symbol] n represents c or deletion (Location 21).

SEQ ID NO:5884 [symbol] n represents c or deletion (Location 21).

SEQ ID NO:5890 [symbol] n represents tc or deletion (Location 21).

SEQ ID NO:5902 [symbol] n represents c or deletion (Location 21).

SEQ ID NO:5904 [symbol] n represents g or deletion (Location 21).

SEQ ID NO:5917 [symbol] n represents a or deletion (Location 21).

SEQ ID NO:5921 [symbol] n represents ca or deletion (Location 21).

SEQ ID NO:5922 [symbol] n represents t or deletion (Location 21).

SEQ ID NO:5934 [symbol] n represents ct or deletion (Location 21).

SEQ ID NO:5965 [symbol] n represents a or deletion (Location 21).

SEQ ID NO:5980 [symbol] n represents t or deletion (Location 21).

SEQ ID NO:5981 [symbol] n represents t or deletion (Location 21).

SEQ ID NO:5981 [symbol] n represents 11 to 13 repeats of t (from Location 21).

SEQ ID NO:5987 [symbol] n represents t or deletion (Location 21).

SEQ ID NO:5989 [symbol] n represents 16 to 8 repeats of t (from Location 21).

SEQ ID NO:5991 [symbol] n represents ctta or deletion (Location 21).

SEQ ID NO:5992 [symbol] n represents c or deletion (Location 21).

SEQ ID NO:5994 [symbol] n represents 10 to 12 repeats of a (from Location 21).

SEQ ID NO:5995 [symbol] n represents gt or deletion (Location 21).

SEQ ID NO:5996 [symbol] n represents a or deletion (Location 21).

SEQ ID NO:6001 [symbol] n represents aatt or deletion (Location 21).

SEQ ID NO:6003 [symbol] n represents t or deletion (Location 21).

SEQ ID NO:6009 [symbol] n represents g or deletion (Location 21).

SEQ ID NO:6021 [symbol] n represents at or deletion (Location 21).

SEQ ID NO:6027 [symbol] n represents 4 to 5 repeats of caaaa (from Location 21).

SEQ ID NO:6036 [symbol] n represents 9 to 10 repeats of a (from Location 21).

SEQ ID NO:6041 [symbol] n represents a or deletion (Location 21).

SEQ ID NO:6047 [symbol] n represents t or deletion (Location 21).

SEQ ID NO:6051 [symbol] n represents t or deletion (Location 21).

SEQ ID NO:6052 [symbol] n represents g or deletion (Location 21).

SEQ ID NO:6060 [symbol] n represents t or deletion (Location 21).

SEQ ID NO:6061 [symbol] n represents t or deletion (Location 21).

SEQ ID NO:6062 [symbol] n represents a or deletion (Location 21).

SEQ ID NO:6072 [symbol] n represents gaa or deletion (Location 21).

SEQ ID NO:6073 [symbol] n represents ag or deletion (Location 21).

SEQ ID NO:6089 [symbol] n represents 9 to 11 repeats of t (from Location 21).

SEQ ID NO:6090 [symbol] n represents a or deletion (Location 21).

SEQ ID NO:6091 [symbol] n represents t or deletion (Location 21).

SEQ ID NO:6173 [symbol] n represents tat or deletion (Location 21).

SEQ ID NO:6174 [symbol] n represents 14 to 17 repeats of ac (from Location 21).

SEQ ID NO:6175 [symbol] n represents 16 to 27 repeats of a (from Location 21).

SEQ ID NO:6176 [symbol] n represents t or deletion (Location 21).

SEQ ID NO:6177 [symbol] n represents 8 to 10 repeats of a (from Location 21).

SEQ ID NO:6178 [symbol] n represents 9 to 11 repeats of gt (from Location 21).

SEQ ID NO:6179 [symbol] n represents aa or deletion (Location 21).

SEQ ID NO:6180 [symbol] n represents t or deletion (Location 21).

SEQ ID NO:6181 [symbol] n represents 8 to 12 repeats of ac (from Location 21).

SEQ ID NO:6182 [symbol] n represents a or deletion (Location 21).

SEQ ID NO:6202 [symbol] n represents agg or deletion (Location 21).

SEQ ID NO:6204 [symbol] n represents 11 to 15 repeats of a (from Location 21).

SEQ ID NO:6205 [symbol] n represents 11 to 14 repeats of a (from Location 21).

SEQ ID NO:6208 [symbol] n represents gt or deletion (Location 21).

SEQ ID NO:6224 [symbol] n represents ta or deletion (Location 21).

SEQ ID NO:6307 [symbol] n represents 16 to 19 repeats of a (from Location 21).

SEQ ID NO:6308 [symbol] n represents aa or deletion (Location 21).

SEQ ID NO:6310 [symbol] n represents t or deletion (Location 21).

SEQ ID NO:6311 [symbol] n represents 10 to 12 repeats of t (from Location 21).

SEQ ID NO:6312 [symbol] n represents aa or deletion (Location 21).

SEQ ID NO:6313 [symbol] n represents ttgacagtccaatat, ttgaca, gtccaatat or deletion (Location 21).

SEQ ID NO:6314 [symbol] n represents cta or deletion (Location 21).

SEQ ID NO:6315 [symbol] n represents a or deletion (Location 21).

SEQ ID NO:6317 [symbol] n represents 9 to 11 repeats of t (From Location 21).

SEQ ID NO:6318 [symbol] n represents c or deletion (Location 21).

SEQ ID NO:6320 [symbol] n represents gagatgttgtggctcacat or deletion (Location 21).

SEQ ID NO:6322 [symbol] n represents cc or deletion (Location 21).

SEQ ID NO:6323 [symbol] n represents act or deletion (Location 21).

SEQ ID NO:6405 [symbol] n represents a or deletion (Location 21).

SEQ ID NO:6415 [symbol] n represents 8 to 11 repeats of t (from Location 21).

SEQ ID NO:6416 [symbol] n represents 10 to 13 repeats of t (from Location 21).

SEQ ID NO:6472 [symbol] n represents g or deletion (Location 21).

SEQ ID NO:6473 [symbol] n represents c or deletion (Location 21).

SEQ ID NO:6554 [symbol] n represents t or deletion (Location 21).

SEQ ID NO:6555 [symbol] n represents 12 to 15 repeats of t (from Location 21).

SEQ ID NO:6609 [symbol] n represents a or deletion (Location 21).

SEQ ID NO:6610 [symbol] n represents at or deletion (Location 21).

SEQ ID NO:6725 [symbol] n represents 16 repeats of cctgc or 16 repeats of cctgt (from Location 21).

SEQ ID NO:6726 [symbol] n represents t or deletion (Location 21).

SEQ ID NO:6728 [symbol] n represents c or deletion (Location 21).

SEQ ID NO:6739 [symbol] n represents acac or deletion (Location 21).

SEQ ID NO:6748 [symbol] n represents gatttgtggtatccag or deletion (Location 21).

SEQ ID NO:6750 [symbol] n represents ag or deletion (Location 21).

SEQ ID NO:6751 [symbol] n represents ta or deletion (Location 21).

SEQ ID NO:6757 [symbol] n represents t or deletion (Location 21).

SEQ ID NO:6759 [symbol] n represents 12 to 14 repeats of gt from Location 21).

SEQ ID NO:6771 [symbol] n represents cagaggct or deletion (Location 21).

SEQ ID NO:6772 [symbol] n represents ct or deletion (Location 21).

SEQ ID NO:6773 [symbol] n represents ag or deletion (Location 21).

SEQ ID NO:6785 [symbol] n represents gtaaa or deletion (Location 21).

SEQ ID NO:6786 [symbol] n represents aaaaa or deletion (Location 21).

SEQ ID NO:6787 [symbol] n represents a or deletion (Location 21).

SEQ ID NO:6828 [symbol] n represents tc or deletion (Location 21).

SEQ ID NO:6830 [symbol] n represents t or deletion (Location 21).

SEQ ID NO:6831 [symbol] n represents t or deletion (Location 21).

SEQ ID NO:6832 [symbol] n represents gaagaaactgttgacagttt or deletion (Location 21).

SEQ ID NO:6833 [symbol] n represents cct or deletion (Location 21).

SEQ ID NO:6834 [symbol] n represents tttc or deletion (Location 21).

SEQ ID NO:6835 [symbol] n represents ttcttttaaaattg or deletion (Location 21).

SEQ ID NO:6837 [symbol] n represents ttcaggccttt or deletion (Location 21).

SEQ ID NO:6839 [symbol] n represents ggcctg or deletion (Location 21).

SEQ ID NO:6841 [symbol] n represents a or deletion (Location 21).

SEQ ID NO:6870 [symbol] n represents 9 to 11 repeats of c (from Location 21).

SEQ ID NO:6871 [symbol] n represents 15 to 21 repeats of a (from Location 21).

SEQ ID NO:6872 [symbol] n represents ggggtggcggggtggg or deletion (Location 21).

SEQ ID NO:6873 [symbol] n represents t or deletion (Location 21).

SEQ ID NO:6874 [symbol] n represents a or deletion (Location 21).

SEQ ID NO:6876 [symbol] n represents a or deletion (Location 21).

SEQ ID NO:6877 [symbol] n represents 10 to 12 repeats of t (from Location 21).

SEQ ID NO:6878 [symbol] n represents tt or deletion (Location 21).

SEQ ID NO:6880 [symbol] n represents tccctccttgaagctgatcgt or deletion (Location 21).

SEQ ID NO:6881 [symbol] n represents 12 to 18 repeats of ca (from Location 21).

SEQ ID NO:6894 [symbol] n represents gtt or deletion (Location 21).

SEQ ID NO:6897 [symbol] n represents ga or deletion (Location 21).

SEQ ID NO:6921 [symbol] n represents t or deletion (Location 21).

SEQ ID NO:6940 [symbol] n represents t or deletion (Location 21).

SEQ ID NO:6941 [symbol] n represents t or deletion (Location 21).

SEQ ID NO:6942 [symbol] n represents t or deletion (Location 21).

SEQ ID NO:6965 [symbol] n represents at or deletion (Location 21).

SEQ ID NO:6966 [symbol] n represents a or deletion (Location 21).

SEQ ID NO:6967 [symbol] n represents c or deletion (Location 21).

SEQ ID NO:6997 [symbol] n represents c or deletion (Location 21).

SEQ ID NO:7005 [symbol] n represents t or deletion (Location 21).

SEQ ID NO:7006 [symbol] n represents ttc or deletion (Location 21).

SEQ ID NO:7017 [symbol] n represents ctt or deletion (Location 21).

SEQ ID NO:7049 [symbol] n represents 8 to 9 repeats of a (from Location 21).

SEQ ID NO:7053 [symbol] n represents 10 to 12 repeats of t (from Location 21).

SEQ ID NO:7059 [symbol] n represents 22 to 25 repeats of t (from Location 21).

SEQ ID NO:7070 [symbol] n represents t or deletion (Location 21).

SEQ ID NO:7073 [symbol] n represents a or deletion (Location 21).

SEQ ID NO:7074 [symbol] n represents a or deletion (Location 21).

SEQ ID NO:7076 [symbol] n represents c or deletion (Location 21).

SEQ ID NO:7077 [symbol] n represents 10 to 12 repeats of t (from Location 21).

SEQ ID NO:7078 [symbol] n represents a or deletion (Location 21).

SEQ ID NO:7079 [symbol] n represents 9 to 11 repeats of t (from Location 21).

SEQ ID NO:7082 [symbol] n represents a or deletion (Location 21).

SEQ ID NO:7085 [symbol] n represents t or deletion (Location 21).

SEQ ID NO:7089 [symbol] n represents a or deletion (Location 21).

SEQ ID NO:7101 [symbol] n represents a or deletion (Location 21).

SEQ ID NO:7105 [symbol] n represents a or deletion (Location 21).

SEQ ID NO:7114 [symbol] n represents 9 to 10 repeats of t (from Location 21).

SEQ ID NO:7115 [symbol] n represents aag or deletion (Location 21).

SEQ ID NO:7117 [symbol] n represents t or deletion (Location 21).

SEQ ID NO:7118 [symbol] n represents t or deletion (Location 21).

SEQ ID NO:7120 [symbol] n represents t or deletion (Location 21).

SEQ ID NO:7121 [symbol] n represents t or deletion (Location 21).

SEQ ID NO:7123 [symbol] n represents t or deletion (Location 21).

SEQ ID NO:7125 [symbol] n represents a or deletion (Location 21).

SEQ ID NO:7127 [symbol] n represents a or deletion (Location 21).

SEQ ID NO:7134 [symbol] n represents 7 to 8 repeats of gt (from Location 21).

SEQ ID NO:7146 [symbol] n represents cat or deletion (Location 21).

SEQ ID NO:7148 [symbol] n represents tc or deletion (Location 21).

SEQ ID NO:7164 [symbol] n represents ca or deletion (Location 21).

SEQ ID NO:7186 [symbol] n represents g or deletion (Location 21).

SEQ ID NO:7209 [symbol] n represents t or deletion (Location 21).

SEQ ID NO:7238 [symbol] n represents gccag or deletion (Location 21).

SEQ ID NO:7278 [symbol] n represents a or deletion (Location 21).

SEQ ID NO:7281 [symbol] n represents g or deletion (Location 21).

SEQ ID NO:7282 [symbol] n represents t or deletion (Location 21).

SEQ ID NO:7287 [symbol] n represents aaa or deletion (Location 21).

SEQ ID NO:7288 [symbol] n represents a or deletion (Location 21).

SEQ ID NO:7299 [symbol] n represents c or deletion (Location 21).

SEQ ID NO:7329 [symbol] n represents 17 to 19 repeats of a (from Location 21).

SEQ ID NO:7332 [symbol] n represents 16 to 18 repeats of a (from Location 21).

SEQ ID NO:7333 [symbol] n represents 4 to 6 repeats of ga (from Location 21).

SEQ ID NO:7346 [symbol] n represents a or deletion (Location 21).

SEQ ID NO:7375 [symbol] n represents 2 to 3 repeats of tc (from Location 21).

SEQ ID NO:7381 [symbol] n represents 6 to 7 repeats of a (from Location 21).

SEQ ID NO:7383 [symbol] n represents 13 to 15 repeats of a (from Location 21).

SEQ ID NO:7385 [symbol] n represents 9 to 10 repeats of t (from Location 21).

SEQ ID NO:7387 [symbol] n represents 12 to 14 repeats of a (from Location 21).

SEQ ID NO:7389 [symbol] n represents 14 to 17 repeats of t (from Location 21).

SEQ ID NO:7390 [symbol] n represents 8 to 9 repeats of a (from Location 21).

SEQ ID NO:7397 [symbol] n represents g or deletion (Location 21).

SEQ ID NO:7417 [symbol] n represents 14 to 17 repeats of t (from Location 21).

SEQ ID NO:7421 [symbol] n represents 7 to 9 repeats of g (from Location 21).

SEQ ID NO:7426 [symbol] n represents 9 to 10 repeats of a (from Location 21).

SEQ ID NO:7434 [symbol] n represents 9 to 10 repeats of a (from Location 21).

SEQ ID NO:7436 [symbol] n represents 6 to 7 repeats of g (from Location 21).

SEQ ID NO:7443 [symbol] n represents g or dele t ion (Location 21).

SEQ ID NO:7458 [symbol] n represents 8 to 9 repeats of a (from Location 21).

SEQ ID NO:7461 [symbol] n represents 4 to 6 repeats of c (from Location 21).

SEQ ID NO:7483 [symbol] n represents ggcgaaggcggcggc or deletion (Location 21).

SEQ ID NO:7485 [symbol] n represents ata or deletion (Location 21).

SEQ ID NO:7488 [symbol] n represents 11 to 12 repeats of t (from Location 21).

SEQ ID NO:7489 [symbol] n represents 12 to 14 repeats of t (from Location 21).

SEQ ID NO:7493 [symbol] n represents 9 to 10 repeats of t (from Location 21).

SEQ ID NO:7495 [symbol] n represents 6 to 7 repeats of ta (from Location 21).

SEQ ID NO:7497 [symbol] n represents tgtatacgtatacatacgtatacatatatacatacgtatata or deletion (Location 21).

SEQ ID NO:7503 [symbol] n represents attt or deletion (Location 21).

SEQ ID NO:7510 [symbol] n represents cct or deletion (Location 21).

SEQ ID NO:7519 [symbol] n represents tgtt or deletion (Location 21).

SEQ ID NO:7520 [symbol] n represents a or deletion (Location 21).

SEQ ID NO:7531 [symbol] n represents 9 to 10 repeats of t (from Location 21).

SEQ ID NO:7538 [symbol] n represents a or deletion (Location 21).

SEQ ID NO:7566 [symbol] n represents a or deletion (Location 21).

SEQ ID NO:7615 [symbol] n represents a or deletion (Location 21).

SEQ ID NO:7649 [symbol] n represents gtg or deletion (Location 21).

SEQ ID NO:7651 [symbol] n represents gg or tggtggggtgga (Location 21).

SEQ ID NO:7667 [symbol] n represents ct or deletion (Location 21).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

What is claimed is:

1. A method of identifying individuals having a polymorphism, comprising;
a. providing nucleic acid from a subject; and
b. detecting the presence of at least one polymorphism in said nucleic acid, said at least one polymorphism selected from the group consisting of polymorphisms found in SEQ ID Nos:1-3360 and 3361-7669.

2. The method of claim 1, further comprising step c) providing a prognosis to said subject based on the presence or absence of said at least one polymorphism.

3. The method of claim 2, wherein said prognosis comprises a genotype relative risk.

4. The method of claim 2, wherein said prognosis comprises a population attributable risk.

5. The method of claim 1, wherein said detecting step comprises use of a hybridization assay.

6. The method of claim 1, wherein said detecting step comprises use of a TAQMAN assay.

7. The method of claim 1, wherein said detecting step comprises use of an invasive cleavage assay.

8. The method of claim 1, wherein said detecting step comprises use of mass spectroscopy.

9. The method of claim 1, wherein said detecting step comprises use of a microarray.

10. The method of claim 1, wherein said detecting step comprises use of a polymerase chain reaction.

11. The method of claim 1, wherein said detecting step comprises use of a rolling circle extension assay.

12. The method of claim 1, wherein said detecting step comprises use of a sequencing assay.

13. The method of claim 1, wherein said detecting step comprises use of a hybridization assay employing a probe complementary to a polymorphism.

14. The method of claim 1, wherein said detecting step comprises use of a bead array assay.

15. The method of claim 1, wherein said detecting step comprises use of a primer extension assay.

16. The method of claim 1, wherein said detecting step comprises use of an enzyme mismatch cleavage assay.

17. The method of claim 1, wherein said detecting step comprises use of a branched hybridization assay.

18. The method of claim 1, wherein said detecting step comprises use of a NASBA assay.

19. The method of claim 1, wherein said detecting step comprises use of a molecular beacon assay.

20. The method of claim 1, wherein said detecting step comprises use of a cycling probe assay.

21. The method of claim 1, wherein said detecting step comprises use of a ligase chain reaction assay.

22. The method of claim 1, wherein said detection step comprises use of a sandwich hybridization assay.

23. A composition comprising a nucleic acid, said nucleic acid comprising a sequence selected from the group consisting of SEQ ID NO: 1-3360 and 3361-7669 or complements thereof.

24. The composition of claim 23, wherein said nucleic acid is 200 or less nucleotides in length.

25. The composition of claim 23, wherein said nucleic acid molecule comprises a label.

26. The composition of claim 23, wherein said nucleic acid comprises a gene sequence.

27. The composition of claim 23, wherein said nucleic acid is attached to a solid support.

28. The vector comprising the nucleic acid of claim 23.

29. A host cell comprising the vector of claim 28.

30. A polypeptide encoded by the nucleic acid of claim 23.

31. A kit for detecting a polymorphism, comprising at least one reagent that specifically detects a polymorphism in a sequence selected from the group consisting of SEQ ID Nos:3360 and 3361-7669.

32. The kit of claim 31, further comprising instructions for determining whether the subject is at increased risk of having a drug metabolism disorder.

33. The kit of claim 31, wherein said at least one reagent comprises a nucleic acid probe.

34. The kit of claim 31, wherein said kit comprises an in vitro diagnostic detection assay.

35. The kit of claim 31, wherein said kit comprises an analyte specific reagent detection assay.

36. The kit of claim 31, wherein said kit comprises a research-use-only detection assay.

37. A method for screening subjects for genetic markers associated with drug metabolizing enzyme(s), comprising:
a) providing a biological sample comprising a nucleic acid from a subject;
b) testing said nucleic acid for a polymorphism in a genetic marker associated with a drug metabolizing enzyme, said genetic marker comprising one or more nucleotide polymorphisms designated by n, said n selected from a base substitution, an insertion, or a deletion found in a sequence selected from the group consisting of SEQ ID Nos:1-3360 and 3361-7669.

38. The method of claim 37, wherein said biological sample is selected from the group consisting of blood, saliva, amniotic fluid, and tissue.

39. The method of claim 37, wherein said subject is a human.

40. The method of claim 37, wherein said nucleic acid comprises DNA.

41. The method of claim 37, wherein said nucleic acid comprises RNA.

42. A composition comprising an array of detection assays, said array comprising a plurality of drug metabolizing enzyme nucleotide polymorphism detection assays, one or more of said detection assays being capable of detecting one or more nucleotide polymorphisms designated by n in SEQ ID Nos:1-3360 and 3361-7669, wherein n represents a base substitution, insertion, or deletion compared to a wild-type sequence.

43. The composition of claim 42, wherein said detection assay is selected from the group consisting of a sequencing assay, a polymerase chain reaction assay, a hybridization assay, a hybridization assay employing a probe complementary to a polymorphism, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, and a sandwich hybridization assay.

44. A composition comprising a detection probe for determining the presense or absence a single nucleotide polymorphism in a gene encoding a drug metabolizing enzyme, said gene comprising a sequence selected from the group consisting of SEQ ID Nos:1-3360 and 3361-7669.

45. A kit comprising the detection probe of claim 44, and at least one PCR primer for amplifying at least a portion of said gene.

46. A method of determining the effectiveness of or side-effect of a drug or treatment protocol, comprising;
a. administering a drug or treatment protocol to one or more subjects;
b. obtaining nucleic acid from said one or more subjects;
c. using a detection assay to detect the presence of at least one polymorphism in said nucleic acid from said one or more of subjects, said at least one polymorphism selected from the group consisting of polymorphisms found in SEQ ID Nos:1-3360 and 3361-7669; and,
d. assigning an effectiveness rating, side-effect rating, or score for said drug or treatment protocol based upon a result of one or more said detection assays.

47. The method of claim 46, wherein said detection assay comprises a hybridization assay.

48. The method of claim 46, wherein said detection assay comprises a TAQMAN assay.

49. The method of claim 46, wherein said detection assay comprises an invasive cleavage assay.

50. The method of claim 46, wherein said detection assay comprises mass spectroscopy.

51. The method of claim 46, wherein said detection assay comprises a micro array.

52. The method of claim 46, wherein said detection assay comprises a polymerase chain reaction.

53. The method of claim 46, wherein said detection assay comprises a rolling circle extension assay.

54. The method of claim 46, wherein said detection assay comprises a sequencing assay.

55. The method of claim 46, wherein said detection assay comprises a hybridization assay employing a probe complementary to a polymorphism.

56. The method of claim 46, wherein said detection assay comprises a bead array assay.

57. The method of claim 46, wherein said detection assay comprises a primer extension assay.

58. The method of claim 46, wherein said detection assay comprises an enzyme mismatch cleavage assay.

59. The method of claim 46, wherein said detection assay comprises a branched hybridization assay.

60. The method of claim 46, wherein said detection assay comprises a NASBA assay.

61. The method of claim 46, wherein said detection assay comprises a molecular beacon assay.

62. The method of claim 46, wherein said detection assay comprises a cycling probe assay.

63. The method of claim 46, wherein said detection assay comprises a ligase chain reaction assay.

64. The method of claim 46, wherein said detection step comprises a sandwich hybridization assay.

65. The method of claim 46, in which said detection assay comprises a kit for detecting a polymorphism, said kit comprising at least one reagent that specifically detects a polymorphism in a sequence selected from the group consisting of SEQ ID Nos:1-3360 and 3361-7669.

66. The method of claim 65, further comprising instructions for determining whether the subject is at increased risk of having a drug metabolism disorder.

67. The method of claim 65, wherein said at least one reagent comprises a nucleic acid probe.

68. The method of claim 65, wherein said kit comprises an analyte specific reagent detection assay.

69. The method of claim 65, wherein said kit comprises a research-use-only detection assay.

70. The method of claim 46, wherein said nucleic acid is obtained from a biological sample, said sample being selected from the group consisting of blood, saliva, amniotic fluid, and tissue.

71. The method of claim 70, wherein said subject is a mammal.

72. The method of claim 70, wherein said nucleic acid comprises DNA.

73. The method of claim 70, wherein said nucleic acid comprises RNA.

74. The method of claim 65, wherein said kit comprises PCR primers.

75. The method of claim 74, in which said kit comprises an array of detection assays, said array comprising a plurality of drug metabolizing enzyme nucleotide polymorphism detection assays, one or more of said detection assays being capable of detecting one or more nucleotide polymorphisms designated by n in SEQ ID Nos:1-3360 and 3361-7669, wherein n represents a base substitution, insertion, or deletion compared to a wild-type sequence.

76. A method of prescribing a drug to or treatment protocol for a subject, comprising;
a. providing nucleic acid from said subject;
b. using a detection assay to detect the presence of at least one polymorphism in said nucleic acid, said at least one polymorphism selected from the group consisting of polymorphisms found in SEQ ID Nos:1-3360 and 3361-7669; and,
c. prescribing said drug or treatment protocol based upon the result of said detection assay.

77. The method of claim 76, further comprising step d) providing a prognosis to said subject based on the presence or absence of said at least one polymorphism.

78. The method of claim 77, wherein said prognosis comprises a genotype relative risk.

79. The method of claim 77, wherein said prognosis comprises a population attributable risk.

80. The method of claim 76, wherein said detection assay comprises a hybridization assay.

81. The method of claim 76, wherein said detection assay comprises a TAQMAN assay.

82. The method of claim 76, wherein said detection assay comprises an invasive cleavage assay.

83. The method of claim 76, wherein said detection assay comprises mass spectroscopy.

84. The method of claim 76, wherein said detection assay comprises a microarray.

85. The method of claim 76, wherein said detection assay comprises a polymerase chain reaction.

86. The method of claim 76, wherein said detection assay comprises a rolling circle extension assay.

87. The method of claim 76, wherein said detection assay comprises a sequencing assay.

88. The method of claim 76, wherein said detection assay comprises a hybridization assay employing a probe complementary to a polymorphism.

89. The method of claim 76, wherein said detection assay comprises a bead array assay.

90. The method of claim 76, wherein said detection assay comprises a primer extension assay.

91. The method of claim 76, wherein said detection assay comprises an enzyme mismatch cleavage assay.

92. The method of claim 76, wherein said detection assay comprises a branched hybridization assay.

93. The method of claim 76, wherein said detection assay comprises a NASBA assay.

94. The method of claim 76, wherein said detection assay comprises a molecular beacon assay.

95. The method of claim 76, wherein said detection assay comprises a cycling probe assay.

96. The method of claim 76, wherein said detection assay comprises a ligase chain reaction assay.

97. The method of claim 76, wherein said detection step comprises a sandwich hybridization assay.

98. The method of claim 76, in which said detection assay comprises a kit for detecting a polymorphism, said kit comprising at least one reagent that specifically detects a polymorphism in a sequence selected from the group consisting of SEQ ID Nos:1-3360 and 3361-7669.

99. The method of claim 98, further comprising instructions for determining whether the subject is at increased risk of having a drug metabolism disorder.

100. The method of claim 99, wherein said at least one reagent comprises a nucleic acid probe.

101. The method of claim 98, wherein said kit comprises an in vitro diagnostic detection assay.

102. The method of claim 98, wherein said kit comprises an analyte specific reagent detection assay.

103. The method of claim 98, wherein said kit comprises a research-use-only detection assay.

104. The method of claim 76, wherein said nucleic acid is obtained from a biological sample, said sample being selected from the group consisting of blood, saliva, amniotic fluid, and tissue.

105. The method of claim 104, wherein said subject is a human.

106. The method of claim 104, wherein said nucleic acid comprises DNA.

107. The method of claim 104, wherein said nucleic acid comprises RNA.

108. The method of claim 104, wherein said kit comprises PCR primers.

109. The method of claim 98, in which said kit comprises an array of detection assays, said array comprising a plurality of drug metabolizing enzyme nucleotide polymorphism detection assays, one or more of said detection assays being capable of detecting one or more nucleotide polymorphisms designated by n in SEQ ID Nos: 1-3360 and 3361-7669, wherein n represents a base substitution, insertion, or deletion compared to a wild-type sequence.

110. The method of claim 109, wherein said detection assay is selected from the group consisting of a sequencing assay, a polymerase chain reaction assay, a hybridization assay, a hybridization assay employing a probe complementary to a polymorphism, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, and a sandwich hybridization assay.

111. A method for generating assay data comprising:
a. obtaining a sample from a subject containing nucleic acid;
b. transferring said sample to a laboratory; and
c. receiving data from said laboratory, wherein said data corresponds to the presence of at least one polymorphism in said nucleic acid, said at least one polymorphism selected from the group consisting of polymorphisms found in SEQ ID Nos:1-3360 and 3361-7669.

112. A data set generated by the method of claim 111.

113. A composition comprising a nucleic acid, said nucleic acid comprising a gene sequence, said gene sequence having at least one polymorphism, said at least one polymorphism selected from the group consisting of polymorphisms found in SEQ ID NOs:1-3360 and 3361-7669.

114. The composition of claim 113, wherein said at least one polymorphism is located in a non-coding portion of said gene.

115. The composition of claim 113, wherein said at least one polymorphism is located in an intron.

116. The composition of claim 113, wherein said at least one polymorphism is located in an exon.

117. The composition of claim 113, wherein said at least one polymorphism is located in a 5′ untranslated portion of said gene.

118. The composition of claim 113, wherein said at least one polymorphism is located in a 3′ untranslated portion of said gene.

119. The composition of claim 113, wherein said at least one polymorphism is located in an untranslated regulatory portion of said gene.

120. The composition of claim 113, wherein said at least one polymorphism is located in a region of said gene affecting splicing.

121. The composition of claim 113, wherein said at least one polymorphism is located in a region of said gene affecting level of transcription of said gene.

122. The composition of claim 113, wherein two or more of said polymorphisms are located in a gene sequence, said gene sequence comprising an intron, an exon, a 5′ untranslated portion of said gene, a 3′ untranslated portion of said gene, a regulatory portion of said gene, a portion of said gene affecting splicing, and a portion of said gene affecting level or transcription of said gene.

123. A composition comprising an oligonucleotide, said oligonucleotide comprising at least one polymorphism, said at least one polymorphism selected from the group consisting of polymorphisms found in SEQ ID Nos:1-3360 and 3361-7669, and said oligonucleotide comprising at least five bases upstream or five bases downstream of said polymorphism.
